SEQUENCE LISTING (673 sequences)

SEQ ID NO: 1 - protein, 141 aa, synthetic construct
MGSSHHHHHHENLYFQGDYKDDDDKGSLEVLFQGPAENESTPIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSK

SEQ ID NO: 2-202 - skipped (201 entries)

SEQ ID NO: 203 - DNA, 20 bp, synthetic construct
  source 1..20: mol_type = other DNA
caagaagcacaagaagcaca

SEQ ID NO: 204 - DNA, 20 bp, synthetic construct
  source 1..20: mol_type = other DNA
cttgtgcttcttgcccatgg

SEQ ID NO: 205 - DNA, 20 bp, synthetic construct
  source 1..20: mol_type = other DNA
cttcttgtgcttcttgccca

SEQ ID NO: 206 - DNA, 20 bp, synthetic construct
  source 1..20: mol_type = other DNA
acaagaagcacaaggccgag

SEQ ID NO: 207 - DNA, 20 bp, synthetic construct
  source 1..20: mol_type = other DNA
ctcgtaggacgagcgccact

SEQ ID NO: 208 - DNA, 20 bp, synthetic construct
  source 1..20: mol_type = other DNA
cgagtggcgctcgtcctacg

SEQ ID NO: 209 - DNA, 20 bp, synthetic construct
  source 1..20: mol_type = other DNA
gagtggcgctcgtcctacga

SEQ ID NO: 210 - DNA, 20 bp, synthetic construct
  source 1..20: mol_type = other DNA
aggcttctccaggggcttgt

SEQ ID NO: 211 - DNA, 20 bp, synthetic construct
  source 1..20: mol_type = other DNA
agattatgccgacaagcccc

SEQ ID NO: 212 - DNA, 20 bp, synthetic construct
  source 1..20: mol_type = other DNA
accttcaggactagctttag

SEQ ID NO: 213 - DNA, 20 bp, synthetic construct
  source 1..20: mol_type = other DNA
agctttagaggcttctccag

SEQ ID NO: 214 - DNA, 20 bp, synthetic construct
  source 1..20: mol_type = other DNA
ctagctttagaggcttctcc

SEQ ID NO: 215 - DNA, 20 bp, synthetic construct
  source 1..20: mol_type = other DNA
tagctttagaggcttctcca

SEQ ID NO: 216 - DNA, 20 bp, synthetic construct
  source 1..20: mol_type = other DNA
ctaaagctagtcctgaaggt

SEQ ID NO: 217 - DNA, 20 bp, synthetic construct
  source 1..20: mol_type = other DNA
gcctctaaagctagtcctga

SEQ ID NO: 218 - DNA, 20 bp, synthetic construct
  source 1..20: mol_type = other DNA
cttcacttcctccgaccttc

SEQ ID NO: 219 - DNA, 20 bp, synthetic construct
  source 1..20: mol_type = other DNA
aagctagtcctgaaggtcgg

SEQ ID NO: 220 - DNA, 20 bp, synthetic construct
  source 1..20: mol_type = other DNA
agtgaagtgactgaactctc

SEQ ID NO: 221 - DNA, 20 bp, synthetic construct
  source 1..20: mol_type = other DNA
gtgactgaactctcaggatc

SEQ ID NO: 222 - DNA, 20 bp, synthetic construct
  source 1..20: mol_type = other DNA
atagtaactggagtcgtggc

SEQ ID NO: 223 - DNA, 20 bp, synthetic construct
  source 1..20: mol_type = other DNA
catcatagtaactggagtcg

SEQ ID NO: 224 - DNA, 20 bp, synthetic construct
  source 1..20: mol_type = other DNA
tgacctgtcatcatagtaac

SEQ ID NO: 225 - DNA, 20 bp, synthetic construct
  source 1..20: mol_type = other DNA
actccagttactatgatgac

SEQ ID NO: 226 - DNA, 20 bp, synthetic construct
  source 1..20: mol_type = other DNA
ctttgtgcctctctcgctca

SEQ ID NO: 227 - DNA, 20 bp, synthetic construct
  source 1..20: mol_type = other DNA
ggtcagaccatgagcgagag

SEQ ID NO: 228 - DNA, 20 bp, synthetic construct
  source 1..20: mol_type = other DNA
gaagaagaagaagtccgaga

SEQ ID NO: 229 - DNA, 20 bp, synthetic construct
  source 1..20: mol_type = other DNA
gtccagatgcttctccttct

SEQ ID NO: 230 - DNA, 20 bp, synthetic construct
  source 1..20: mol_type = other DNA
gtccgagaaggagaagcatc

SEQ ID NO: 231 - DNA, 20 bp, synthetic construct
  source 1..20: mol_type = other DNA
ggagaagcatctggacgatg

SEQ ID NO: 232 - DNA, 20 bp, synthetic construct
  source 1..20: mol_type = other DNA
tgaggaaagaaggaagcgaa

SEQ ID NO: 233 - DNA, 20 bp, synthetic construct
  source 1..20: mol_type = other DNA
atctggacgatgaggaaaga

SEQ ID NO: 234 - DNA, 20 bp, synthetic construct
  source 1..20: mol_type = other DNA
agaagaagcggaagcgagag

SEQ ID NO: 235 - DNA, 20 bp, synthetic construct
  source 1..20: mol_type = other DNA
gaagaagcggaagcgagaga

SEQ ID NO: 236 - DNA, 20 bp, synthetic construct
  source 1..20: mol_type = other DNA
ccgcccaggaagagaagaag

SEQ ID NO: 237 - DNA, 20 bp, synthetic construct
  source 1..20: mol_type = other DNA
agagagggagcactgtgaca

SEQ ID NO: 238 - DNA, 20 bp, synthetic construct
  source 1..20: mol_type = other DNA
agggagcactgtgacacgga

SEQ ID NO: 239 - DNA, 20 bp, synthetic construct
  source 1..20: mol_type = other DNA
gagggagcactgtgacacgg

SEQ ID NO: 240 - DNA, 20 bp, synthetic construct
  source 1..20: mol_type = other DNA
gcactgtgacacggagggag

SEQ ID NO: 241 - DNA, 20 bp, synthetic construct
  source 1..20: mol_type = other DNA
gaggctgacgactttgatcc

SEQ ID NO: 242 - DNA, 20 bp, synthetic construct
  source 1..20: mol_type = other DNA
aggctgacgactttgatcct

SEQ ID NO: 243 - DNA, 20 bp, synthetic construct
  source 1..20: mol_type = other DNA
tccacctccaccttcttccc

SEQ ID NO: 244 - DNA, 20 bp, synthetic construct
  source 1..20: mol_type = other DNA
cgactttgatcctgggaaga

SEQ ID NO: 245 - DNA, 20 bp, synthetic construct
  source 1..20: mol_type = other DNA
ctttgatcctgggaagaagg

SEQ ID NO: 246 - DNA, 20 bp, synthetic construct
  source 1..20: mol_type = other DNA
tgatcctgggaagaaggtgg

SEQ ID NO: 247 - DNA, 20 bp, synthetic construct
  source 1..20: mol_type = other DNA
tcctgggaagaaggtggagg

SEQ ID NO: 248 - DNA, 20 bp, synthetic construct
  source 1..20: mol_type = other DNA
cggactggccgatctggggg

SEQ ID NO: 249 - DNA, 20 bp, synthetic construct
  source 1..20: mol_type = other DNA
acgctcggactggccgatct

SEQ ID NO: 250 - DNA, 20 bp, synthetic construct
  source 1..20: mol_type = other DNA
aggtggagccgcccccagat

SEQ ID NO: 251 - DNA, 20 bp, synthetic construct
  source 1..20: mol_type = other DNA
cgctcggactggccgatctg

SEQ ID NO: 252 - DNA, 20 bp, synthetic construct
  source 1..20: mol_type = other DNA
gctcggactggccgatctgg

SEQ ID NO: 253 - DNA, 20 bp, synthetic construct
  source 1..20: mol_type = other DNA
cacgctcggactggccgatc

SEQ ID NO: 254 - DNA, 20 bp, synthetic construct
  source 1..20: mol_type = other DNA
tgtgtccggcacgctcggac

SEQ ID NO: 255 - DNA, 20 bp, synthetic construct
  source 1..20: mol_type = other DNA
ctggctgtgtccggcacgct

SEQ ID NO: 256 - DNA, 20 bp, synthetic construct
  source 1..20: mol_type = other DNA
atcggccagtccgagcgtgc

SEQ ID NO: 257 - DNA, 20 bp, synthetic construct
  source 1..20: mol_type = other DNA
cacccttgcctggctgtgtc

SEQ ID NO: 258 - DNA, 20 bp, synthetic construct
  source 1..20: mol_type = other DNA
cgagcgtgccggacacagcc

SEQ ID NO: 259 - DNA, 20 bp, synthetic construct
  source 1..20: mol_type = other DNA
tgttccaggagttgctgaat

SEQ ID NO: 260 - DNA, 20 bp, synthetic construct
  source 1..20: mol_type = other DNA
cacacctattcagcaactcc

SEQ ID NO: 261 - DNA, 20 bp, synthetic construct
  source 1..20: mol_type = other DNA
gctggcggaggaagtgttcc

SEQ ID NO: 262 - DNA, 20 bp, synthetic construct
  source 1..20: mol_type = other DNA
tttacctctgaagctggcgg

SEQ ID NO: 263 - DNA, 20 bp, synthetic construct
  source 1..20: mol_type = other DNA
ccccggtttacctctgaagc

SEQ ID NO: 264 - DNA, 20 bp, synthetic construct
  source 1..20: mol_type = other DNA
acttcctccgccagcttcag

SEQ ID NO: 265 - DNA, 20 bp, synthetic construct
  source 1..20: mol_type = other DNA
caggaaaagcaaaaaatcca

SEQ ID NO: 266 - DNA, 20 bp, synthetic construct
  source 1..20: mol_type = other DNA
gctttcagaaaagatcccca

SEQ ID NO: 267 - DNA, 20 bp, synthetic construct
  source 1..20: mol_type = other DNA
aggaaaagcaaaaaatccat

SEQ ID NO: 268 - DNA, 20 bp, synthetic construct
  source 1..20: mol_type = other DNA
ggaaaagcaaaaaatccatg

SEQ ID NO: 269 - DNA, 20 bp, synthetic construct
  source 1..20: mol_type = other DNA
ggagcaattgcatccgtgac

SEQ ID NO: 270 - DNA, 20 bp, synthetic construct
  source 1..20: mol_type = other DNA
gtcacggatgcaattgctcc

SEQ ID NO: 271 - DNA, 20 bp, synthetic construct
  source 1..20: mol_type = other DNA
tttattatcattgaatatcc

SEQ ID NO: 272 - DNA, 20 bp, synthetic construct
  source 1..20: mol_type = other DNA
aatgataataaaacatccca

SEQ ID NO: 273 - DNA, 20 bp, synthetic construct
  source 1..20: mol_type = other DNA
ataaaacatcccatggattt

SEQ ID NO: 274 - DNA, 20 bp, synthetic construct
  source 1..20: mol_type = other DNA
ttcatggtgccaaaatccat

SEQ ID NO: 275 - DNA, 20 bp, synthetic construct
  source 1..20: mol_type = other DNA
tttcatggtgccaaaatcca

SEQ ID NO: 276 - DNA, 20 bp, synthetic construct
  source 1..20: mol_type = other DNA
taatgaatacaagtcagtta

SEQ ID NO: 277 - DNA, 20 bp, synthetic construct
  source 1..20: mol_type = other DNA
caagtcagttacggaattta

SEQ ID NO: 278 - DNA, 20 bp, synthetic construct
  source 1..20: mol_type = other DNA
ataatgcaatgacatacaat

SEQ ID NO: 279 - DNA, 20 bp, synthetic construct
  source 1..20: mol_type = other DNA
aacttgtagtacacggtatc

SEQ ID NO: 280 - DNA, 20 bp, synthetic construct
  source 1..20: mol_type = other DNA
cttcgccaacttgtagtaca

SEQ ID NO: 281 - DNA, 20 bp, synthetic construct
  source 1..20: mol_type = other DNA
agataccgtgtactacaagt

SEQ ID NO: 282 - DNA, 20 bp, synthetic construct
  source 1..20: mol_type = other DNA
gcgaagaagatccttcacgc

SEQ ID NO: 283 - DNA, 20 bp, synthetic construct
  source 1..20: mol_type = other DNA
tcatcttaaagcctgcgtga

SEQ ID NO: 284 - DNA, 20 bp, synthetic construct
  source 1..20: mol_type = other DNA
ttctcagcaggcagctcttt

SEQ ID NO: 285 - DNA, 20 bp, synthetic construct
  source 1..20: mol_type = other DNA
caatgaagatacagctgttg

SEQ ID NO: 286 - DNA, 20 bp, synthetic construct
  source 1..20: mol_type = other DNA
actggtacaacttcagggac

SEQ ID NO: 287 - DNA, 20 bp, synthetic construct
  source 1..20: mol_type = other DNA
cttgtactggtacaacttca

SEQ ID NO: 288 - DNA, 20 bp, synthetic construct
  source 1..20: mol_type = other DNA
acttgtactggtacaacttc

SEQ ID NO: 289 - DNA, 20 bp, synthetic construct
  source 1..20: mol_type = other DNA
ttggcagtttctacttgtac

SEQ ID NO: 290 - DNA, 20 bp, synthetic construct
  source 1..20: mol_type = other DNA
tacctgataacttctctact

SEQ ID NO: 291 - DNA, 20 bp, synthetic construct
  source 1..20: mol_type = other DNA
agccgagtagagaagttatc

SEQ ID NO: 292 - DNA, 20 bp, synthetic construct
  source 1..20: mol_type = other DNA
agctgcatgtttgagcctga

SEQ ID NO: 293 - DNA, 20 bp, synthetic construct
  source 1..20: mol_type = other DNA
gctgcatgtttgagcctgaa

SEQ ID NO: 294 - DNA, 20 bp, synthetic construct
  source 1..20: mol_type = other DNA
aagctgcaggcattcccttc

SEQ ID NO: 295 - DNA, 20 bp, synthetic construct
  source 1..20: mol_type = other DNA
ggtactgtccgtcaagctgc

SEQ ID NO: 296 - DNA, 20 bp, synthetic construct
  source 1..20: mol_type = other DNA
agggaatgcctgcagcttga

SEQ ID NO: 297 - DNA, 20 bp, synthetic construct
  source 1..20: mol_type = other DNA
cttgacggacagtaccgcag

SEQ ID NO: 298 - DNA, 20 bp, synthetic construct
  source 1..20: mol_type = other DNA
cgccagcacgtgctcctctg

SEQ ID NO: 299 - DNA, 20 bp, synthetic construct
  source 1..20: mol_type = other DNA
taccgcagaggagcacgtgc

SEQ ID NO: 300 - DNA, 20 bp, synthetic construct
  source 1..20: mol_type = other DNA
agaggagcacgtgctggcgc

SEQ ID NO: 301 - DNA, 20 bp, synthetic construct
  source 1..20: mol_type = other DNA
ggagcacgtgctggcgctgg

SEQ ID NO: 302 - DNA, 20 bp, synthetic construct
  source 1..20: mol_type = other DNA
agcacgcagctgacgaagct

SEQ ID NO: 303 - DNA, 20 bp, synthetic construct
  source 1..20: mol_type = other DNA
gcacgcagctgacgaagctc

SEQ ID NO: 304 - DNA, 20 bp, synthetic construct
  source 1..20: mol_type = other DNA
cagctgacgaagctcgggac

SEQ ID NO: 305 - DNA, 20 bp, synthetic construct
  source 1..20: mol_type = other DNA
aagctcgggacaggatcaac

SEQ ID NO: 306 - DNA, 20 bp, synthetic construct
  source 1..20: mol_type = other DNA
ccttgccgcctgggaggaac

SEQ ID NO: 307 - DNA, 20 bp, synthetic construct
  source 1..20: mol_type = other DNA
aggatcaaccggttcctccc

SEQ ID NO: 308 - DNA, 20 bp, synthetic construct
  source 1..20: mol_type = other DNA
atcaaccggttcctcccagg

SEQ ID NO: 309 - DNA, 20 bp, synthetic construct
  source 1..20: mol_type = other DNA
gcactaccttgccgcctggg

SEQ ID NO: 310 - DNA, 20 bp, synthetic construct
  source 1..20: mol_type = other DNA
agagcactaccttgccgcct

SEQ ID NO: 311 - DNA, 20 bp, synthetic construct
  source 1..20: mol_type = other DNA
ccggttcctcccaggcggca

SEQ ID NO: 312 - DNA, 20 bp, synthetic construct
  source 1..20: mol_type = other DNA
tcctcttcagatagcccatc

SEQ ID NO: 313 - DNA, 20 bp, synthetic construct
  source 1..20: mol_type = other DNA
atgggctatctgaagaggaa

SEQ ID NO: 314 - DNA, 20 bp, synthetic construct
  source 1..20: mol_type = other DNA
gggctatctgaagaggaacg

SEQ ID NO: 315 - DNA, 20 bp, synthetic construct
  source 1..20: mol_type = other DNA
tgggctatctgaagaggaac

SEQ ID NO: 316 - DNA, 20 bp, synthetic construct
  source 1..20: mol_type = other DNA
tatctgaagaggaacgggga

SEQ ID NO: 317 - DNA, 20 bp, synthetic construct
  source 1..20: mol_type = other DNA
atctgaagaggaacggggac

SEQ ID NO: 318 - DNA, 20 bp, synthetic construct
  source 1..20: mol_type = other DNA
tgttgaccacgctgtagagc

SEQ ID NO: 319 - DNA, 20 bp, synthetic construct
  source 1..20: mol_type = other DNA
gctctacagcgtggtcaaca

SEQ ID NO: 320 - DNA, 20 bp, synthetic construct
  source 1..20: mol_type = other DNA
cgggagcctgctctacagcg

SEQ ID NO: 321 - DNA, 20 bp, synthetic construct
  source 1..20: mol_type = other DNA
cgtggtcaacacggccgagc

SEQ ID NO: 322 - DNA, 20 bp, synthetic construct
  source 1..20: mol_type = other DNA
cccaccatcagcgtccggct

SEQ ID NO: 323 - DNA, 20 bp, synthetic construct
  source 1..20: mol_type = other DNA
acggccgagccggacgctga

SEQ ID NO: 324 - DNA, 20 bp, synthetic construct
  source 1..20: mol_type = other DNA
gggcacccaccatcagcgtc

SEQ ID NO: 325 - DNA, 20 bp, synthetic construct
  source 1..20: mol_type = other DNA
gccgagccggacgctgatgg

SEQ ID NO: 326 - DNA, 20 bp, synthetic construct
  source 1..20: mol_type = other DNA
ccatgtccgtgttgcagagg

SEQ ID NO: 327 - DNA, 20 bp, synthetic construct
  source 1..20: mol_type = other DNA
ccgagccggacgctgatggt

SEQ ID NO: 328 - DNA, 20 bp, synthetic construct
  source 1..20: mol_type = other DNA
cgagctcaagtccaccgggt

SEQ ID NO: 329 - DNA, 20 bp, synthetic construct
  source 1..20: mol_type = other DNA
gcgagctcaagtccaccggg

SEQ ID NO: 330 - DNA, 20 bp, synthetic construct
  source 1..20: mol_type = other DNA
agagcgagctcaagtccacc

SEQ ID NO: 331 - DNA, 20 bp, synthetic construct
  source 1..20: mol_type = other DNA
gagagcgagctcaagtccac

SEQ ID NO: 332 - DNA, 20 bp, synthetic construct
  source 1..20: mol_type = other DNA
gaagcctgggagtagcttac

SEQ ID NO: 333 - DNA, 20 bp, synthetic construct
  source 1..20: mol_type = other DNA
ctctccagtaagctactccc

SEQ ID NO: 334 - DNA, 20 bp, synthetic construct
  source 1..20: mol_type = other DNA
agcccagcgtggtgaagcct

SEQ ID NO: 335 - DNA, 20 bp, synthetic construct
  source 1..20: mol_type = other DNA
aagcccagcgtggtgaagcc

SEQ ID NO: 336 - DNA, 20 bp, synthetic construct
  source 1..20: mol_type = other DNA
actcccaggcttcaccacgc

SEQ ID NO: 337 - DNA, 20 bp, synthetic construct
  source 1..20: mol_type = other DNA
ctcccaggcttcaccacgct

SEQ ID NO: 338 - DNA, 20 bp, synthetic construct
  source 1..20: mol_type = other DNA
ctcgtctttgaagcccagcg

SEQ ID NO: 339 - DNA, 20 bp, synthetic construct
  source 1..20: mol_type = other DNA
cactggagagaaaggtgact

SEQ ID NO: 340 - DNA, 20 bp, synthetic construct
  source 1..20: mol_type = other DNA
gcactggagagaaaggtgac

SEQ ID NO: 341 - DNA, 20 bp, synthetic construct
  source 1..20: mol_type = other DNA
agtagtggcactggagagaa

SEQ ID NO: 342 - DNA, 20 bp, synthetic construct
  source 1..20: mol_type = other DNA
cgaaagcgcagtagtggcac

SEQ ID NO: 343 - DNA, 20 bp, synthetic construct
  source 1..20: mol_type = other DNA
ctgcatcgaaagcgcagtag

SEQ ID NO: 344 - DNA, 20 bp, synthetic construct
  source 1..20: mol_type = other DNA
atgcagaataattcagtatt

SEQ ID NO: 345 - DNA, 20 bp, synthetic construct
  source 1..20: mol_type = other DNA
agtatttggcgacttgaagt

SEQ ID NO: 346 - DNA, 20 bp, synthetic construct
  source 1..20: mol_type = other DNA
cgacttgaagtcggacgaga

SEQ ID NO: 347 - DNA, 20 bp, synthetic construct
  source 1..20: mol_type = other DNA
gagctgctctactcagccta

SEQ ID NO: 348 - DNA, 20 bp, synthetic construct
  source 1..20: mol_type = other DNA
cacgcctgtctcatctccgt

SEQ ID NO: 349 - DNA, 20 bp, synthetic construct
  source 1..20: mol_type = other DNA
tcagcctacggagatgagac

SEQ ID NO: 350 - DNA, 20 bp, synthetic construct
  source 1..20: mol_type = other DNA
caggcgtgcagtgtgcgctg

SEQ ID NO: 351 - DNA, 20 bp, synthetic construct
  source 1..20: mol_type = other DNA
ccgcggcccctctagcctgc

SEQ ID NO: 352 - DNA, 20 bp, synthetic construct
  source 1..20: mol_type = other DNA
catccttcacaaactcctgc

SEQ ID NO: 353 - DNA, 20 bp, synthetic construct
  source 1..20: mol_type = other DNA
tagcctgcaggagtttgtga

SEQ ID NO: 354 - DNA, 20 bp, synthetic construct
  source 1..20: mol_type = other DNA
caggagtttgtgaaggatgc

SEQ ID NO: 355 - DNA, 20 bp, synthetic construct
  source 1..20: mol_type = other DNA
aggagtttgtgaaggatgct

SEQ ID NO: 356 - DNA, 20 bp, synthetic construct
  source 1..20: mol_type = other DNA
tgggagctacagcaagaaag

SEQ ID NO: 357 - DNA, 20 bp, synthetic construct
  source 1..20: mol_type = other DNA
gagctacagcaagaaagtgg

SEQ ID NO: 358 - DNA, 20 bp, synthetic construct
  source 1..20: mol_type = other DNA
gaaagtggtggacgacctcc

SEQ ID NO: 359 - DNA, 20 bp, synthetic construct
  source 1..20: mol_type = other DNA
cgcctgtgatctggtccagg

SEQ ID NO: 360 - DNA, 20 bp, synthetic construct
  source 1..20: mol_type = other DNA
ctccgcctgtgatctggtcc

SEQ ID NO: 361 - DNA, 20 bp, synthetic construct
  source 1..20: mol_type = other DNA
gacctcctggaccagatcac

SEQ ID NO: 362 - DNA, 20 bp, synthetic construct
  source 1..20: mol_type = other DNA
ctcctggaccagatcacagg

SEQ ID NO: 363 - DNA, 20 bp, synthetic construct
  source 1..20: mol_type = other DNA
gctggaagagcgtcctagag

SEQ ID NO: 364 - DNA, 20 bp, synthetic construct
  source 1..20: mol_type = other DNA
tgcagcccacctgcttcagc

SEQ ID NO: 365 - DNA, 20 bp, synthetic construct
  source 1..20: mol_type = other DNA
gacgctcttccagctgaagc

SEQ ID NO: 366 - DNA, 20 bp, synthetic construct
  source 1..20: mol_type = other DNA
ctcttccagctgaagcaggt

SEQ ID NO: 367 - DNA, 20 bp, synthetic construct
  source 1..20: mol_type = other DNA
gctcttccagctgaagcagg

SEQ ID NO: 368 - DNA, 20 bp, synthetic construct
  source 1..20: mol_type = other DNA
cctccagatgaagccaaggt

SEQ ID NO: 369 - DNA, 20 bp, synthetic construct
  source 1..20: mol_type = other DNA
gcttcatctggaggcttcat

SEQ ID NO: 370 - DNA, 20 bp, synthetic construct
  source 1..20: mol_type = other DNA
ggcttcatctggaggcttca

SEQ ID NO: 371 - DNA, 20 bp, synthetic construct
  source 1..20: mol_type = other DNA
cttaccttggcttcatctgg

SEQ ID NO: 372 - DNA, 20 bp, synthetic construct
  source 1..20: mol_type = other DNA
aaacttaccttggcttcatc

SEQ ID NO: 373 - DNA, 20 bp, synthetic construct
  source 1..20: mol_type = other DNA
gaagcctccagatgaagcca

SEQ ID NO: 374 - DNA, 20 bp, synthetic construct
  source 1..20: mol_type = other DNA
tcctagggtgtccccaacct

SEQ ID NO: 375 - DNA, 20 bp, synthetic construct
  source 1..20: mol_type = other DNA
cctagggtgtccccaacctg

SEQ ID NO: 376 - DNA, 20 bp, synthetic construct
  source 1..20: mol_type = other DNA
gtgtctgtctccacaggttg

SEQ ID NO: 377 - DNA, 20 bp, synthetic construct
  source 1..20: mol_type = other DNA
tgtgtctgtctccacaggtt

SEQ ID NO: 378 - DNA, 20 bp, synthetic construct
  source 1..20: mol_type = other DNA
ccacaggttggggacaccct

SEQ ID NO: 379 - DNA, 20 bp, synthetic construct
  source 1..20: mol_type = other DNA
agagctgctgctgtctccta

SEQ ID NO: 380 - DNA, 20 bp, synthetic construct
  source 1..20: mol_type = other DNA
cagagctgctgctgtctcct

SEQ ID NO: 381 - DNA, 20 bp, synthetic construct
  source 1..20: mol_type = other DNA
agacagcagcagctctgttc

SEQ ID NO: 382 - DNA, 20 bp, synthetic construct
  source 1..20: mol_type = other DNA
atccacagaaacgtcgggat

SEQ ID NO: 383 - DNA, 20 bp, synthetic construct
  source 1..20: mol_type = other DNA
gagatatccacagaaacgtc

SEQ ID NO: 384 - DNA, 20 bp, synthetic construct
  source 1..20: mol_type = other DNA
ggagatatccacagaaacgt

SEQ ID NO: 385 - DNA, 20 bp, synthetic construct
  source 1..20: mol_type = other DNA
gtcctatcccgacgtttctg

SEQ ID NO: 386 - DNA, 20 bp, synthetic construct
  source 1..20: mol_type = other DNA
tctccatgctcagctctctg

SEQ ID NO: 387 - DNA, 20 bp, synthetic construct
  source 1..20: mol_type = other DNA
ctcacccagagagctgagca

SEQ ID NO: 388 - DNA, 20 bp, synthetic construct
  source 1..20: mol_type = other DNA
atctccatgctcagctctct

SEQ ID NO: 389 - DNA, 20 bp, synthetic construct
  source 1..20: mol_type = other DNA
tatctccatgctcagctctc

SEQ ID NO: 390 - DNA, 20 bp, synthetic construct
  source 1..20: mol_type = other DNA
atgtcctgtttacacaggga

SEQ ID NO: 391 - DNA, 20 bp, synthetic construct
  source 1..20: mol_type = other DNA
ttacacagggaaggtgaaga

SEQ ID NO: 392 - DNA, 20 bp, synthetic construct
  source 1..20: mol_type = other DNA
agttcaaatggctgtcgtca

SEQ ID NO: 393 - DNA, 20 bp, synthetic construct
  source 1..20: mol_type = other DNA
tgacgacagccatttgaact

SEQ ID NO: 394 - DNA, 20 bp, synthetic construct
  source 1..20: mol_type = other DNA
aagttcaaatggctgtcgtc

SEQ ID NO: 395 - DNA, 20 bp, synthetic construct
  source 1..20: mol_type = other DNA
tcgtctcatccaagttcaaa

SEQ ID NO: 396 - DNA, 20 bp, synthetic construct
  source 1..20: mol_type = other DNA
tgagacgacgaagctcctgc

SEQ ID NO: 397 - DNA, 20 bp, synthetic construct
  source 1..20: mol_type = other DNA
gtgcttcgtgcaggtcctgc

SEQ ID NO: 398 - DNA, 20 bp, synthetic construct
  source 1..20: mol_type = other DNA
gcaggacctgcacgaagcac

SEQ ID NO: 399 - DNA, 20 bp, synthetic construct
  source 1..20: mol_type = other DNA
gctccgcctgtgcttcgtgc

SEQ ID NO: 400 - DNA, 20 bp, synthetic construct
  source 1..20: mol_type = other DNA
ggacctgcacgaagcacagg

SEQ ID NO: 401 - DNA, 20 bp, synthetic construct
  source 1..20: mol_type = other DNA
cacgaagcacaggcggagcg

SEQ ID NO: 402 - DNA, 20 bp, synthetic construct
  source 1..20: mol_type = other DNA
aggcggagcgcggcggctct

SEQ ID NO: 403 - DNA, 20 bp, synthetic construct
  source 1..20: mol_type = other DNA
agggagctgaggttggacga

SEQ ID NO: 404 - DNA, 20 bp, synthetic construct
  source 1..20: mol_type = other DNA
gttggacagggagctgaggt

SEQ ID NO: 405 - DNA, 20 bp, synthetic construct
  source 1..20: mol_type = other DNA
aggcgttggacagggagctg

SEQ ID NO: 406 - DNA, 20 bp, synthetic construct
  source 1..20: mol_type = other DNA
ccctctcggaggcgttggac

SEQ ID NO: 407 - DNA, 20 bp, synthetic construct
  source 1..20: mol_type = other DNA
cctctcggaggcgttggaca

SEQ ID NO: 408 - DNA, 20 bp, synthetic construct
  source 1..20: mol_type = other DNA
ctggtccctctcggaggcgt

SEQ ID NO: 409 - DNA, 20 bp, synthetic construct
  source 1..20: mol_type = other DNA
ccctgtccaacgcctccgag

SEQ ID NO: 410 - DNA, 20 bp, synthetic construct
  source 1..20: mol_type = other DNA
cctgtccaacgcctccgaga

SEQ ID NO: 411 - DNA, 20 bp, synthetic construct
  source 1..20: mol_type = other DNA
gtggtgctggtccctctcgg

SEQ ID NO: 412 - DNA, 20 bp, synthetic construct
  source 1..20: mol_type = other DNA
caggtggtgctggtccctct

SEQ ID NO: 413 - DNA, 20 bp, synthetic construct
  source 1..20: mol_type = other DNA
gcatctcacccaggtggtgc

SEQ ID NO: 414 - DNA, 20 bp, synthetic construct
  source 1..20: mol_type = other DNA
cgagagggaccagcaccacc

SEQ ID NO: 415 - DNA, 20 bp, synthetic construct
  source 1..20: mol_type = other DNA
gagagggaccagcaccacct

SEQ ID NO: 416 - DNA, 20 bp, synthetic construct
  source 1..20: mol_type = other DNA
gtgggggcatctcacccagg

SEQ ID NO: 417 - DNA, 20 bp, synthetic construct
  source 1..20: mol_type = other DNA
ccccgacactcaggcgagaa

SEQ ID NO: 418 - DNA, 20 bp, synthetic construct
  source 1..20: mol_type = other DNA
tccccgacactcaggcgaga

SEQ ID NO: 419 - DNA, 20 bp, synthetic construct
  source 1..20: mol_type = other DNA
agcccttctcgcctgagtgt

SEQ ID NO: 420 - DNA, 20 bp, synthetic construct
  source 1..20: mol_type = other DNA
ctggctgctccccgacactc

SEQ ID NO: 421 - DNA, 20 bp, synthetic construct
  source 1..20: mol_type = other DNA
cccttctcgcctgagtgtcg

SEQ ID NO: 422 - DNA, 20 bp, synthetic construct
  source 1..20: mol_type = other DNA
gcccttctcgcctgagtgtc

SEQ ID NO: 423 - DNA, 20 bp, synthetic construct
  source 1..20: mol_type = other DNA
taggggtcgtgggtgacgtc

SEQ ID NO: 424 - DNA, 20 bp, synthetic construct
  source 1..20: mol_type = other DNA
aagaaactcataggggtcgt

SEQ ID NO: 425 - DNA, 20 bp, synthetic construct
  source 1..20: mol_type = other DNA
gaagaaactcataggggtcg

SEQ ID NO: 426 - DNA, 20 bp, synthetic construct
  source 1..20: mol_type = other DNA
gagactgaagaaactcatag

SEQ ID NO: 427 - DNA, 20 bp, synthetic construct
  source 1..20: mol_type = other DNA
ggagactgaagaaactcata

SEQ ID NO: 428 - DNA, 20 bp, synthetic construct
  source 1..20: mol_type = other DNA
tggagactgaagaaactcat

SEQ ID NO: 429 - DNA, 20 bp, synthetic construct
  source 1..20: mol_type = other DNA
tcttcagtctccagagcctg

SEQ ID NO: 430 - DNA, 20 bp, synthetic construct
  source 1..20: mol_type = other DNA
ttggcagaggccgcaggctc

SEQ ID NO: 431 - DNA, 20 bp, synthetic construct
  source 1..20: mol_type = other DNA
taggtcttggcagaggccgc

SEQ ID NO: 432 - DNA, 20 bp, synthetic construct
  source 1..20: mol_type = other DNA
ctagagttaggtcttggcag

SEQ ID NO: 433 - DNA, 20 bp, synthetic construct
  source 1..20: mol_type = other DNA
ggtggtctagagttaggtct

SEQ ID NO: 434 - DNA, 20 bp, synthetic construct
  source 1..20: mol_type = other DNA
gtagcgaacgtgtccggcgt

SEQ ID NO: 435 - DNA, 20 bp, synthetic construct
  source 1..20: mol_type = other DNA
gaccggaacgatctcgcgta

SEQ ID NO: 436 - DNA, 20 bp, synthetic construct
  source 1..20: mol_type = other DNA
ggcagtcgttcggttgatat

SEQ ID NO: 437 - DNA, 20 bp, synthetic construct
  source 1..20: mol_type = other DNA
gcttgagcacatacgcgaat

SEQ ID NO: 438 - DNA, 20 bp, synthetic construct
  source 1..20: mol_type = other DNA
gtggtagaataacgtattac

SEQ ID NO: 439 - DNA, 20 bp, synthetic construct
  source 1..20: mol_type = other DNA
gtcatacatggataaggcta

SEQ ID NO: 440 - DNA, 20 bp, synthetic construct
  source 1..20: mol_type = other DNA
gatacacgaagcatcactag

SEQ ID NO: 441 - DNA, 20 bp, synthetic construct
  source 1..20: mol_type = other DNA
gaacgttggcactacttcac

SEQ ID NO: 442 - DNA, 20 bp, synthetic construct
  source 1..20: mol_type = other DNA
gatccatgtaatgcgttcga

SEQ ID NO: 443 - DNA, 20 bp, synthetic construct
  source 1..20: mol_type = other DNA
gtcgtgaagtgcattcgatc

SEQ ID NO: 444 - DNA, 20 bp, synthetic construct
  source 1..20: mol_type = other DNA
gttcgactcgcgtgaccgta

SEQ ID NO: 445 - DNA, 20 bp, synthetic construct
  source 1..20: mol_type = other DNA
gaatctaccgcagcggttcg

SEQ ID NO: 446 - DNA, 20 bp, synthetic construct
  source 1..20: mol_type = other DNA
gaagtgacgtcgattcgata

SEQ ID NO: 447 - DNA, 20 bp, synthetic construct
  source 1..20: mol_type = other DNA
gcggtgtatgacaaccgccg

SEQ ID NO: 448 - DNA, 20 bp, synthetic construct
  source 1..20: mol_type = other DNA
gtaccgcgcctgaagttcgc

SEQ ID NO: 449 - DNA, 20 bp, synthetic construct
  source 1..20: mol_type = other DNA
gcagctcgtgtgtcgtactc

SEQ ID NO: 450 - DNA, 20 bp, synthetic construct
  source 1..20: mol_type = other DNA
gcgccttaagagtactcatc

SEQ ID NO: 451 - DNA, 20 bp, synthetic construct
  source 1..20: mol_type = other DNA
gagtgtcgtcgttgctccta

SEQ ID NO: 452 - DNA, 20 bp, synthetic construct
  source 1..20: mol_type = other DNA
gcagctcgacctcaagccgt

SEQ ID NO: 453 - DNA, 20 bp, synthetic construct
  source 1..20: mol_type = other DNA
gtatcctgacctacgcgctg

SEQ ID NO: 454 - DNA, 20 bp, synthetic construct
  source 1..20: mol_type = other DNA
gtgtatctcagcacgctaac

SEQ ID NO: 455 - DNA, 20 bp, synthetic construct
  source 1..20: mol_type = other DNA
gtcgtcatacaacggcaacg

SEQ ID NO: 456 - DNA, 20 bp, synthetic construct
  source 1..20: mol_type = other DNA
gtcgtgcgcttccggcggta

SEQ ID NO: 457 - DNA, 20 bp, synthetic construct
  source 1..20: mol_type = other DNA
gcggtcctcagtaagcgcgt

SEQ ID NO: 458 - DNA, 20 bp, synthetic construct
  source 1..20: mol_type = other DNA
gctctgctgcggaaggattc

SEQ ID NO: 459 - DNA, 20 bp, synthetic construct
  source 1..20: mol_type = other DNA
gcatggaggagcgtcgcaga

SEQ ID NO: 460 - DNA, 20 bp, synthetic construct
  source 1..20: mol_type = other DNA
gtagcgcgcgtaggagtggc

SEQ ID NO: 461 - DNA, 20 bp, synthetic construct
  source 1..20: mol_type = other DNA
gatcacctgcattcgtacac

SEQ ID NO: 462 - DNA, 20 bp, synthetic construct
  source 1..20: mol_type = other DNA
gcacacctagatatcgaatg

SEQ ID NO: 463 - DNA, 20 bp, synthetic construct
  source 1..20: mol_type = other DNA
gttgatcaacgcgcttcgcg

SEQ ID NO: 464 - DNA, 20 bp, synthetic construct
  source 1..20: mol_type = other DNA
gcgtctcactcactccatcg

SEQ ID NO: 465 - DNA, 20 bp, synthetic construct
  source 1..20: mol_type = other DNA
gccgaccaacgtcagcggta

SEQ ID NO: 466 - DNA, 20 bp, synthetic construct
  source 1..20: mol_type = other DNA
ggatacggtgcgtcaatcta

SEQ ID NO: 467 - DNA, 20 bp, synthetic construct
  source 1..20: mol_type = other DNA
gaatccagtggcggcgacaa

SEQ ID NO: 468 - DNA, 20 bp, synthetic construct
  source 1..20: mol_type = other DNA
gcactgtcagtgcaacgata

SEQ ID NO: 469 - DNA, 20 bp, synthetic construct
  source 1..20: mol_type = other DNA
gcgatcctcaagtatgctca

SEQ ID NO: 470 - DNA, 20 bp, synthetic construct
  source 1..20: mol_type = other DNA
gctaatatcgacacggccgc

SEQ ID NO: 471 - DNA, 20 bp, synthetic construct
  source 1..20: mol_type = other DNA
ggagatgcatcgaagtcgat

SEQ ID NO: 472 - DNA, 20 bp, synthetic construct
  source 1..20: mol_type = other DNA
ggatgcactccatctcgtct

SEQ ID NO: 473 - DNA, 20 bp, synthetic construct
  source 1..20: mol_type = other DNA
gtgccgagtaataacgcgag

SEQ ID NO: 474 - DNA, 20 bp, synthetic construct
  source 1..20: mol_type = other DNA
gagattccgatgtaacgtac

SEQ ID NO: 475 - DNA, 20 bp, synthetic construct
  source 1..20: mol_type = other DNA
gtcgtcacgagcaggattgc

SEQ ID NO: 476 - DNA, 20 bp, synthetic construct
  source 1..20: mol_type = other DNA
gcgttagtcacttagctcga

SEQ ID NO: 477 - DNA, 20 bp, synthetic construct
  source 1..20: mol_type = other DNA
gttcacacggtgtcggatag

SEQ ID NO: 478 - DNA, 20 bp, synthetic construct
  source 1..20: mol_type = other DNA
ggataggtgaccttagtacg

SEQ ID NO: 479 - DNA, 20 bp, synthetic construct
  source 1..20: mol_type = other DNA
gtatgagtcaagctaatgcg

SEQ ID NO: 480 - DNA, 20 bp, synthetic construct
  source 1..20: mol_type = other DNA
gcaactattggaatacgtga

SEQ ID NO: 481 - DNA, 20 bp, synthetic construct
  source 1..20: mol_type = other DNA
gttaccttcgctcgtctata

SEQ ID NO: 482 - DNA, 20 bp, synthetic construct
  source 1..20: mol_type = other DNA
gtaccgagcaccacaggccg

SEQ ID NO: 483 - DNA, 20 bp, synthetic construct
  source 1..20: mol_type = other DNA
gtcagccatcggatagagat

SEQ ID NO: 484 - DNA, 20 bp, synthetic construct
  source 1..20: mol_type = other DNA
gtacggcactcctagccgct

SEQ ID NO: 485 - DNA, 20 bp, synthetic construct
  source 1..20: mol_type = other DNA
ggtcctgtcgtatgcttgca

SEQ ID NO: 486 - DNA, 20 bp, synthetic construct
  source 1..20: mol_type = other DNA
gccgcaatatatgcggtaag

SEQ ID NO: 487 - DNA, 20 bp, synthetic construct
  source 1..20: mol_type = other DNA
gcgcacgtataatcctgcgt

SEQ ID NO: 488 - DNA, 20 bp, synthetic construct
  source 1..20: mol_type = other DNA
gtgcacaacacgatccacga

SEQ ID NO: 489 - DNA, 20 bp, synthetic construct
  source 1..20: mol_type = other DNA
gcacaatgttgacgtaagtg

SEQ ID NO: 490 - DNA, 20 bp, synthetic construct
  source 1..20: mol_type = other DNA
gtaagatgctgctcaccgtg

SEQ ID NO: 491 - DNA, 20 bp, synthetic construct
  source 1..20: mol_type = other DNA
gtcggtgatccaacgtatcg

SEQ ID NO: 492 - DNA, 20 bp, synthetic construct
  source 1..20: mol_type = other DNA
gagctagtaggacgcaagac

SEQ ID NO: 493 - DNA, 20 bp, synthetic construct
  source 1..20: mol_type = other DNA
gtacgtggaagcttgtggcc

SEQ ID NO: 494 - DNA, 20 bp, synthetic construct
  source 1..20: mol_type = other DNA
gagaactgccagttctcgat

SEQ ID NO: 495 - DNA, 20 bp, synthetic construct
  source 1..20: mol_type = other DNA
gccattcggcgcggcacttc

SEQ ID NO: 496 - DNA, 20 bp, synthetic construct
  source 1..20: mol_type = other DNA
gcacacgaccaatccgcttc

SEQ ID NO: 497 - DNA, 20 bp, synthetic construct
  source 1..20: mol_type = other DNA
gaggtgatcgattaagtaca

SEQ ID NO: 498 - DNA, 20 bp, synthetic construct
  source 1..20: mol_type = other DNA
gtcactcgcagacgcctaac

SEQ ID NO: 499 - DNA, 20 bp, synthetic construct
  source 1..20: mol_type = other DNA
gcgctacggaatcatacgtt

SEQ ID NO: 500 - DNA, 20 bp, synthetic construct
  source 1..20: mol_type = other DNA
ggtaggacctcacggcgcgc

SEQ ID NO: 501 - DNA, 20 bp, synthetic construct
  source 1..20: mol_type = other DNA
gaactgcatcttgttgtagt

SEQ ID NO: 502 - DNA, 20 bp, synthetic construct
  source 1..20: mol_type = other DNA
gatcctgatccggcggcgcg

SEQ ID NO: 503 - DNA, 20 bp, synthetic construct
  source 1..20: mol_type = other DNA
ggtatgcgcgatcctgagtt

SEQ ID NO: 504 - DNA, 20 bp, synthetic construct
  source 1..20: mol_type = other DNA
gcggagctagagagcggtca

SEQ ID NO: 505 - DNA, 20 bp, synthetic construct
  source 1..20: mol_type = other DNA
gaatggcaattacggctgat

SEQ ID NO: 506 - DNA, 20 bp, synthetic construct
  source 1..20: mol_type = other DNA
gtatggtgagtagtcgcttg

SEQ ID NO: 507 - DNA, 20 bp, synthetic construct
  source 1..20: mol_type = other DNA
gtgtaattgcgtctagtcgg

SEQ ID NO: 508 - DNA, 20 bp, synthetic construct
  source 1..20: mol_type = other DNA
ggtcctggcgaggagccttg

SEQ ID NO: 509 - DNA, 20 bp, synthetic construct
  source 1..20: mol_type = other DNA
gaagataagtcgctgtctcg

SEQ ID NO: 510 - DNA, 20 bp, synthetic construct
  source 1..20: mol_type = other DNA
gtcggcgttctgttgtgact

SEQ ID NO: 511 - DNA, 20 bp, synthetic construct
  source 1..20: mol_type = other DNA
gaggcaagccgttaggtgta

SEQ ID NO: 512 - DNA, 20 bp, synthetic construct
  source 1..20: mol_type = other DNA
gcggatccagatctcattcg

SEQ ID NO: 513 - DNA, 20 bp, synthetic construct
  source 1..20: mol_type = other DNA
ggaacataggagcacgtagt

SEQ ID NO: 514 - DNA, 20 bp, synthetic construct
  source 1..20: mol_type = other DNA
gtcatcattatggcgtaagg

SEQ ID NO: 515 - DNA, 20 bp, synthetic construct
  source 1..20: mol_type = other DNA
gcgactagcgccatgagcgg

SEQ ID NO: 516 - DNA, 20 bp, synthetic construct
  source 1..20: mol_type = other DNA
ggcgaagttcgacatgacac

SEQ ID NO: 517 - DNA, 20 bp, synthetic construct
  source 1..20: mol_type = other DNA
gctgtcgtgtggaggctatg

SEQ ID NO: 518 - DNA, 20 bp, synthetic construct
  source 1..20: mol_type = other DNA
gcggagagcattgacctcat

SEQ ID NO: 519 - DNA, 20 bp, synthetic construct
  source 1..20: mol_type = other DNA
gactaatggaccaagtcagt

SEQ ID NO: 520 - DNA, 20 bp, synthetic construct
  source 1..20: mol_type = other DNA
gcggattagaggtaatgcgg

SEQ ID NO: 521 - DNA, 20 bp, synthetic construct
  source 1..20: mol_type = other DNA
gccgacggcaatcagtacgc

SEQ ID NO: 522 - DNA, 20 bp, synthetic construct
  source 1..20: mol_type = other DNA
gtaacctctcgagcgataga

SEQ ID NO: 523 - DNA, 20 bp, synthetic construct
  source 1..20: mol_type = other DNA
gacttgtatgtggcttacgg

SEQ ID NO: 524 - DNA, 20 bp, synthetic construct
  source 1..20: mol_type = other DNA
gtcactgtggtcgaacatgt

SEQ ID NO: 525 - DNA, 20 bp, synthetic construct
  source 1..20: mol_type = other DNA
gtactccaatccgcgatgac

SEQ ID NO: 526 - DNA, 20 bp, synthetic construct
  source 1..20: mol_type = other DNA
gcgttggcacgatgttacgg

SEQ ID NO: 527 - DNA, 20 bp, synthetic construct
  source 1..20: mol_type = other DNA
gaaccagccggctagtatga

SEQ ID NO: 528 - DNA, 20 bp, synthetic construct
  source 1..20: mol_type = other DNA
gtatactagctaaccacacg

SEQ ID NO: 529 - DNA, 20 bp, synthetic construct
  source 1..20: mol_type = other DNA
gaatcggaatagttgattcg

SEQ ID NO: 530 - DNA, 20 bp, synthetic construct
  source 1..20: mol_type = other DNA
gagcacttgcatgaggcggt

SEQ ID NO: 531 - DNA, 20 bp, synthetic construct
  source 1..20: mol_type = other DNA
gaacggcgatgaagccagcc

SEQ ID NO: 532 - DNA, 20 bp, synthetic construct
  source 1..20: mol_type = other DNA
gcaaccgagatgagaggttc

SEQ ID NO: 533 - DNA, 20 bp, synthetic construct
  source 1..20: mol_type = other DNA
gcaagatcaatatgcgtgat

SEQ ID NO: 534 - DNA, 20 bp, synthetic construct
  source 1..20: mol_type = other DNA
acggaggctaagcgtcgcaa

SEQ ID NO: 535 - DNA, 20 bp, synthetic construct
  source 1..20: mol_type = other DNA
cgcttccgcggcccgttcaa

SEQ ID NO: 536 - DNA, 20 bp, synthetic construct
  source 1..20: mol_type = other DNA
atcgtttccgcttaacggcg

SEQ ID NO: 537 - DNA, 20 bp, synthetic construct
  source 1..20: mol_type = other DNA
gtaggcgcgccgctctctac

SEQ ID NO: 538 - DNA, 20 bp, synthetic construct
  source 1..20: mol_type = other DNA
ccatatcggggcgagacatg

SEQ ID NO: 539 - DNA, 20 bp, synthetic construct
  source 1..20: mol_type = other DNA
tactaacgccgctcctacag

SEQ ID NO: 540 - DNA, 20 bp, synthetic construct
  source 1..20: mol_type = other DNA
tgaggatcatgtcgagcgcc

SEQ ID NO: 541 - DNA, 20 bp, synthetic construct
  source 1..20: mol_type = other DNA
gggcccgcataggatatcgc

SEQ ID NO: 542 - DNA, 20 bp, synthetic construct
  source 1..20: mol_type = other DNA
tagacaaccgcggagaatgc

SEQ ID NO: 543 - DNA, 20 bp, synthetic construct
  source 1..20: mol_type = other DNA
acgggcggctatcgctgact

SEQ ID NO: 544 - DNA, 20 bp, synthetic construct
  source 1..20: mol_type = other DNA
cgcggaaattttaccgacga

SEQ ID NO: 545 - DNA, 20 bp, synthetic construct
  source 1..20: mol_type = other DNA
cttacaatcgtcggtccaat

SEQ ID NO: 546 - DNA, 20 bp, synthetic construct
  source 1..20: mol_type = other DNA
gcgtgcgtcccgggttaccc

SEQ ID NO: 547 - DNA, 20 bp, synthetic construct
  source 1..20: mol_type = other DNA
cggagtaacaagcggacgga

SEQ ID NO: 548 - DNA, 20 bp, synthetic construct
  source 1..20: mol_type = other DNA
cgagtgttatacgcaccgtt

SEQ ID NO: 549 - DNA, 20 bp, synthetic construct
  source 1..20: mol_type = other DNA
cgactaaccggaaacttttt

SEQ ID NO: 550 - DNA, 20 bp, synthetic construct
  source 1..20: mol_type = other DNA
caacgggttctcccggctac

SEQ ID NO: 551 - DNA, 20 bp, synthetic construct
  source 1..20: mol_type = other DNA
caggagtcgccgatacgcgt

SEQ ID NO: 552 - DNA, 20 bp, synthetic construct
  source 1..20: mol_type = other DNA
ttcacgtcgtctcgcgacca

SEQ ID NO: 553 - DNA, 20 bp, synthetic construct
  source 1..20: mol_type = other DNA
gtgtcggattccgccgctta

SEQ ID NO: 554 - DNA, 20 bp, synthetic construct
  source 1..20: mol_type = other DNA
cacgaactcacaccgcgcga

SEQ ID NO: 555 - DNA, 20 bp, synthetic construct
  source 1..20: mol_type = other DNA
cgctagtacgctcctctata

SEQ ID NO: 556 - DNA, 20 bp, synthetic construct
  source 1..20: mol_type = other DNA
tcgcgcttgggttatacgct

SEQ ID NO: 557 - DNA, 20 bp, synthetic construct
  source 1..20: mol_type = other DNA
ctatctcgagtggtaatgcg

SEQ ID NO: 558 - DNA, 20 bp, synthetic construct
  source 1..20: mol_type = other DNA
aatcgactcgaacttcgtgt

SEQ ID NO: 559 - DNA, 20 bp, synthetic construct
  source 1..20: mol_type = other DNA
cccgatggactataccgaac

SEQ ID NO: 560 - DNA, 20 bp, synthetic construct
  source 1..20: mol_type = other DNA
acgttcgagtacgaccagct

SEQ ID NO: 561 - DNA, 20 bp, synthetic construct
  source 1..20: mol_type = other DNA
cgcgacgactcaacctagtc

SEQ ID NO: 562 - DNA, 20 bp, synthetic construct
  source 1..20: mol_type = other DNA
ggtcaccgatcgagagctag

SEQ ID NO: 563 - DNA, 20 bp, synthetic construct
  source 1..20: mol_type = other DNA
ctcaaccgaccgtatggtca

SEQ ID NO: 564 - DNA, 20 bp, synthetic construct
  source 1..20: mol_type = other DNA
cgtattcgactctcaacgcg

SEQ ID NO: 565 - DNA, 20 bp, synthetic construct
  source 1..20: mol_type = other DNA
ctagccgcccagatcgagcc

SEQ ID NO: 566 - DNA, 20 bp, synthetic construct
  source 1..20: mol_type = other DNA
gaatcgaccgacactaatgt

SEQ ID NO: 567 - DNA, 20 bp, synthetic construct
  source 1..20: mol_type = other DNA
acttcagttcggcgtagtca

SEQ ID NO: 568 - DNA, 20 bp, synthetic construct
  source 1..20: mol_type = other DNA
gtgcgatgtcgcttcaacgt

SEQ ID NO: 569 - DNA, 20 bp, synthetic construct
  source 1..20: mol_type = other DNA
cgcctaatttccggatcaat

SEQ ID NO: 570 - DNA, 20 bp, synthetic construct
  source 1..20: mol_type = other DNA
cgtggccggaaccgtcatag

SEQ ID NO: 571 - DNA, 20 bp, synthetic construct
  source 1..20: mol_type = other DNA
accctccgaatcgtaacgga

SEQ ID NO: 572 - DNA, 20 bp, synthetic construct
  source 1..20: mol_type = other DNA
aaacggtacgacagcgtgtg

SEQ ID NO: 573 - DNA, 20 bp, synthetic construct
  source 1..20: mol_type = other DNA
acatagtcgacggctcgatt

SEQ ID NO: 574 - DNA, 20 bp, synthetic construct
  source 1..20: mol_type = other DNA
gatggcgcttcagtcgtcgg

SEQ ID NO: 575 - DNA, 20 bp, synthetic construct
  source 1..20: mol_type = other DNA
ataatccggaaacgctcgac

SEQ ID NO: 576 - DNA, 20 bp, synthetic construct
  source 1..20: mol_type = other DNA
cgccgggctgacaattaacg

SEQ ID NO: 577 - DNA, 20 bp, synthetic construct
  source 1..20: mol_type = other DNA
cgtcgccatatgccggtggc

SEQ ID NO: 578 - DNA, 20 bp, synthetic construct
  source 1..20: mol_type = other DNA
cgggcctataacaccatcga

SEQ ID NO: 579 - DNA, 20 bp, synthetic construct
  source 1..20: mol_type = other DNA
cgccgttccgagatacttga

SEQ ID NO: 580 - DNA, 20 bp, synthetic construct
  source 1..20: mol_type = other DNA
cgggacgtcgcgaaaatgta

SEQ ID NO: 581 - DNA, 20 bp, synthetic construct
  source 1..20: mol_type = other DNA
tcggcatacgggacacacgc

SEQ ID NO: 582 - DNA, 20 bp, synthetic construct
  source 1..20: mol_type = other DNA
agctccatcgccgcgataat

SEQ ID NO: 583 - DNA, 20 bp, synthetic construct
  source 1..20: mol_type = other DNA
atcgtatcatcagctagcgc

SEQ ID NO: 584 - DNA, 20 bp, synthetic construct
  source 1..20: mol_type = other DNA
tcgatcgaggttgcattcgg

SEQ ID NO: 585 - DNA, 20 bp, synthetic construct
  source 1..20: mol_type = other DNA
ctcgacagttcgtcccgagc

SEQ ID NO: 586 - DNA, 20 bp, synthetic construct
  source 1..20: mol_type = other DNA
cggtagtattaatcgctgac

SEQ ID NO: 587 - DNA, 20 bp, synthetic construct
  source 1..20: mol_type = other DNA
tgaacgcgtgtttccttgca

SEQ ID NO: 588 - DNA, 20 bp, synthetic construct
  source 1..20: mol_type = other DNA
cgacgctaggtaacgtagag

SEQ ID NO: 589 - DNA, 20 bp, synthetic construct
  source 1..20: mol_type = other DNA
cattgttgagcgggcgcgct

SEQ ID NO: 590 - DNA, 20 bp, synthetic construct
  source 1..20: mol_type = other DNA
ccgctattgaaaccgcccac

SEQ ID NO: 591 - DNA, 20 bp, synthetic construct
  source 1..20: mol_type = other DNA
agacacgtcaccggtcaaaa

SEQ ID NO: 592 - DNA, 20 bp, synthetic construct
  source 1..20: mol_type = other DNA
tttacgatctagcggcgtag

SEQ ID NO: 593 - DNA, 20 bp, synthetic construct
  source 1..20: mol_type = other DNA
ttcgcacgattgcaccttgg

SEQ ID NO: 594 - DNA, 20 bp, synthetic construct
  source 1..20: mol_type = other DNA
ggttagagactaggcgcgcg

SEQ ID NO: 595 - DNA, 20 bp, synthetic construct
  source 1..20: mol_type = other DNA
cctccgtgctaacgcggacg

SEQ ID NO: 596 - DNA, 20 bp, synthetic construct
  source 1..20: mol_type = other DNA
ttatcgcgtagtgctgacgt

SEQ ID NO: 597 - DNA, 20 bp, synthetic construct
  source 1..20: mol_type = other DNA
tacgcttgcgtttagcgtcc

SEQ ID NO: 598 - DNA, 20 bp, synthetic construct
  source 1..20: mol_type = other DNA
cgcggcccacgcgtcatcgc

SEQ ID NO: 599 - DNA, 20 bp, synthetic construct
  source 1..20: mol_type = other DNA
agctcgccatgtcggttctc

SEQ ID NO: 600 - DNA, 20 bp, synthetic construct
  source 1..20: mol_type = other DNA
aactagcccgagcagcttcg

SEQ ID NO: 601 - DNA, 20 bp, synthetic construct
  source 1..20: mol_type = other DNA
cgcaaggtgtcggtaaccct

SEQ ID NO: 602 - DNA, 20 bp, synthetic construct
  source 1..20: mol_type = other DNA
cttcgacgccatcgtgctca

SEQ ID NO: 603 - DNA, 20 bp, synthetic construct
  source 1..20: mol_type = other DNA
tcctggataccgcgtggtta

SEQ ID NO: 604 - DNA, 20 bp, synthetic construct
  source 1..20: mol_type = other DNA
atagccgccgctcattactt

SEQ ID NO: 605 - DNA, 20 bp, synthetic construct
  source 1..20: mol_type = other DNA
gtcgtccgggattacaaaat

SEQ ID NO: 606 - DNA, 20 bp, synthetic construct
  source 1..20: mol_type = other DNA
taatgctgcacacgccgaat

SEQ ID NO: 607 - DNA, 20 bp, synthetic construct
  source 1..20: mol_type = other DNA
tatcgcttccgattagtccg

SEQ ID NO: 608 - DNA, 20 bp, synthetic construct
  source 1..20: mol_type = other DNA
gtaccataccgcgtaccctt

SEQ ID NO: 609 - DNA, 20 bp, synthetic construct
  source 1..20: mol_type = other DNA
taagatccgcgggtggcaac

SEQ ID NO: 610 - DNA, 20 bp, synthetic construct
  source 1..20: mol_type = other DNA
gtagacgtcgtgagcttcac

SEQ ID NO: 611 - DNA, 20 bp, synthetic construct
  source 1..20: mol_type = other DNA
tcgcggacatagggctctaa

SEQ ID NO: 612 - DNA, 20 bp, synthetic construct
  source 1..20: mol_type = other DNA
agcgcagatagcgcgtatca

SEQ ID NO: 613 - DNA, 20 bp, synthetic construct
  source 1..20: mol_type = other DNA
gttcgcttcgtaacgaggaa

SEQ ID NO: 614 - DNA, 20 bp, synthetic construct
  source 1..20: mol_type = other DNA
gacccccgataacttttgac

SEQ ID NO: 615 - DNA, 20 bp, synthetic construct
  source 1..20: mol_type = other DNA
acgtccatactgtcggctac

SEQ ID NO: 616 - DNA, 20 bp, synthetic construct
  source 1..20: mol_type = other DNA
gtaccattgccggctcccta

SEQ ID NO: 617 - DNA, 20 bp, synthetic construct
  source 1..20: mol_type = other DNA
tggttccgtaggtcggtata

SEQ ID NO: 618 - DNA, 20 bp, synthetic construct
  source 1..20: mol_type = other DNA
tctggcttgacacgaccgtt

SEQ ID NO: 619 - DNA, 20 bp, synthetic construct
  source 1..20: mol_type = other DNA
cgctaggtccggtaagtgcg

SEQ ID NO: 620 - DNA, 20 bp, synthetic construct
  source 1..20: mol_type = other DNA
agcacgtaatgtccgtggat

SEQ ID NO: 621 - DNA, 20 bp, synthetic construct
  source 1..20: mol_type = other DNA
aaggcgcgcgaatgtggcag

SEQ ID NO: 622 - DNA, 20 bp, synthetic construct
  source 1..20: mol_type = other DNA
actgcggagcgcccaatatc

SEQ ID NO: 623 - DNA, 20 bp, synthetic construct
  source 1..20: mol_type = other DNA
cgtcgagtgctcgaactcca

SEQ ID NO: 624 - DNA, 20 bp, synthetic construct
  source 1..20: mol_type = other DNA
tcgcagcggcgtgggatcgg

SEQ ID NO: 625 - DNA, 20 bp, synthetic construct
  source 1..20: mol_type = other DNA
atctgtcctaattcggatcg

SEQ ID NO: 626 - DNA, 20 bp, synthetic construct
  source 1..20: mol_type = other DNA
tgcggcgtaatgcttgaaag

SEQ ID NO: 627 - DNA, 20 bp, synthetic construct
  source 1..20: mol_type = other DNA
cgaacttaatcccgtggcaa

SEQ ID NO: 628 - DNA, 20 bp, synthetic construct
  source 1..20: mol_type = other DNA
gccgtgttgctggatacgcc

SEQ ID NO: 629 - DNA, 20 bp, synthetic construct
  source 1..20: mol_type = other DNA
taccctccggatacggactg

SEQ ID NO: 630 - DNA, 20 bp, synthetic construct
  source 1..20: mol_type = other DNA
ccgttggactatggcgggtc

SEQ ID NO: 631 - DNA, 20 bp, synthetic construct
  source 1..20: mol_type = other DNA
gtacggggcgatcatccaca

SEQ ID NO: 632 - DNA, 20 bp, synthetic construct
  source 1..20: mol_type = other DNA
aagagtagtagacgcccggg

SEQ ID NO: 633 - DNA, 20 bp, synthetic construct
  source 1..20: mol_type = other DNA
aagagcgaatcgatttcgtg

SEQ ID NO: 634 - DNA, 20 bp, synthetic construct
  source 1..20: mol_type = other DNA
tcaacaccagtgcctgacgg

SEQ ID NO: 635 - DNA, 20 bp, synthetic construct
  source 1..20: mol_type = other DNA
aaagtagcttcactctctcg

SEQ ID NO: 636 - DNA, 20 bp, synthetic construct
  source 1..20: mol_type = other DNA
gagccaaccaatagatgtcc

SEQ ID NO: 637 - DNA, 20 bp, synthetic construct
  source 1..20: mol_type = other DNA
gcgccgccatgaacctagag

SEQ ID NO: 638 - DNA, 20 bp, synthetic construct
  source 1..20: mol_type = other DNA
acaaaggttggaacagaacc

SEQ ID NO: 639 - DNA, 20 bp, synthetic construct
  source 1..20: mol_type = other DNA
ggtgaccgggttattgatgt

SEQ ID NO: 640 - DNA, 20 bp, synthetic construct
  source 1..20: mol_type = other DNA
ttagtggaggactacagagc

SEQ ID NO: 641 - DNA, 20 bp, synthetic construct
  source 1..20: mol_type = other DNA
acatatagcccgtaaagctg

SEQ ID NO: 642 - DNA, 20 bp, synthetic construct
  source 1..20: mol_type = other DNA
cgttggcgatgatctccacg

SEQ ID NO: 643 - DNA, 20 bp, synthetic construct
  source 1..20: mol_type = other DNA
tggccttttctacctcgcgc

SEQ ID NO: 644 - DNA, 20 bp, synthetic construct
  source 1..20: mol_type = other DNA
aatggagatactcatctggg

SEQ ID NO: 645 - DNA, 20 bp, synthetic construct
  source 1..20: mol_type = other DNA
gaagcccgtccagaaagtgt

SEQ ID NO: 646 - DNA, 20 bp, synthetic construct
  source 1..20: mol_type = other DNA
caatctgaggaactccacga

SEQ ID NO: 647 - DNA, 20 bp, synthetic construct
  source 1..20: mol_type = other DNA
aggctgcggcgcccacgaga

SEQ ID NO: 648 - DNA, 20 bp, synthetic construct
  source 1..20: mol_type = other DNA
actttgaccaggccttgcta

SEQ ID NO: 649 - DNA, 20 bp, synthetic construct
  source 1..20: mol_type = other DNA
accttccataactgccacgc

SEQ ID NO: 650 - DNA, 20 bp, synthetic construct
  source 1..20: mol_type = other DNA
cgaggcgtacatacccaagg

SEQ ID NO: 651 - DNA, 20 bp, synthetic construct
  source 1..20: mol_type = other DNA
atggtacggccaaatcaaga

SEQ ID NO: 652 - DNA, 20 bp, synthetic construct
  source 1..20: mol_type = other DNA
tcttgtaatcccatacgcgt

SEQ ID NO: 653 - DNA, 20 bp, synthetic construct
  source 1..20: mol_type = other DNA
attcacaggacacagagaat

SEQ ID NO: 654 - DNA, 20 bp, synthetic construct
  source 1..20: mol_type = other DNA
ccagggctccatcctcaaga

SEQ ID NO: 655 - DNA, 20 bp, synthetic construct
  source 1..20: mol_type = other DNA
tgagctgcaccaaagagacg

SEQ ID NO: 656 - DNA, 20 bp, synthetic construct
  source 1..20: mol_type = other DNA
atgtctgcagatgtacccct

SEQ ID NO: 657 - DNA, 20 bp, synthetic construct
  source 1..20: mol_type = other DNA
cgaagataacgcggatacct

SEQ ID NO: 658 - DNA, 20 bp, synthetic construct
  source 1..20: mol_type = other DNA
gctgcaggccgagtacaccg

SEQ ID NO: 659 - DNA, 20 bp, synthetic construct
  source 1..20: mol_type = other DNA
acaagtgggaggcttacctg

SEQ ID NO: 660 - DNA, 20 bp, synthetic construct
  source 1..20: mol_type = other DNA
gcagcgtacagggatgatca

SEQ ID NO: 661 - DNA, 20 bp, synthetic construct
  source 1..20: mol_type = other DNA
gcagtagcgcttcaggccca

SEQ ID NO: 662 - DNA, 20 bp, synthetic construct
  source 1..20: mol_type = other DNA
caaatggtggggtaacagaa

SEQ ID NO: 663 - DNA, 20 bp, synthetic construct
  source 1..20: mol_type = other DNA
gaaaggaactggctaccgtt

SEQ ID NO: 664 - DNA, 20 bp, synthetic construct
  source 1..20: mol_type = other DNA
agggcttccgttacaagatg

SEQ ID NO: 665 - DNA, 20 bp, synthetic construct
  source 1..20: mol_type = other DNA
gaacaagcaacacctaaaag

SEQ ID NO: 666 - DNA, 20 bp, synthetic construct
  source 1..20: mol_type = other DNA
tgaggagaaggaacggctca

SEQ ID NO: 667 - DNA, 20 bp, synthetic construct
  source 1..20: mol_type = other DNA
ggaagaatgcagagtataag

SEQ ID NO: 668 - DNA, 20 bp, synthetic construct
  source 1..20: mol_type = other DNA
ggaatttgaggaactcctga

SEQ ID NO: 669 - DNA, 20 bp, synthetic construct
  source 1..20: mol_type = other DNA
gctcaccggccatccaggaa

SEQ ID NO: 670 - DNA, 20 bp, synthetic construct
  source 1..20: mol_type = other DNA
actggccaggaacgatgcga

SEQ ID NO: 671 - DNA, 20 bp, synthetic construct
  source 1..20: mol_type = other DNA
gcagctccaagatcttccca

SEQ ID NO: 672 - DNA, 20 bp, synthetic construct
  source 1..20: mol_type = other DNA
gaatgagtacacagaacgga

SEQ ID NO: 673 - DNA, 20 bp, synthetic construct
  source 1..20: mol_type = other DNA
ggagcaggacaaggtcgggg

(19) United States
(12) Patent Application Publication
(10) Pub. No.: US 2026/0008778 A1
(43) Pub. Date: Jan. 8, 2026

(54) COMPOUNDS AND USES THEREOF

(71) Applicant: FOGHORN THERAPEUTICS INC., Cambridge, MA (US)

(72) Inventors: Sabine K. RUPPEL, Cambridge, MA (US); Zhaoxia YANG, Belmont, MA (US); Jason T. LOWE, East Bridgewater, MA (US); Johannes H. VOIGT, Cambridge, MA (US); Matthew Russell NETHERTON, Cambridge, MA (US)

(21) Appl. No.: 19/084,277

(22) Filed: Mar. 19, 2025

Related U.S. Application Data
(63) Continuation of application No. 18/216,392, filed on Jun. 29, 2023, now Pat. No. 12,351,579.
Application No. 18/216,392 is a continuation of application No. 17/425,153, now abandoned.
(60) Provisional application No. 62/798,374, filed on Jan. 29, 2019.
(60) Provisional application No. 62/881,195, filed on Jul. 31, 2019.

(51) Int. Cl. C07D 471/04; A61P 35/00; C07D 401/14; C07D 471/10; C07D 487/04; C07D 487/10; C07D 498/10; C07D 519/00

(52) U.S. Cl. CPC C07D 471/04; A61P 35/00; C07D 401/14; C07D 471/10; C07D 487/04; C07D 487/10; C07D 498/10; C07D 519/00

(57) ABSTRACT

The present invention relates to compositions and methods for the treatment of BAF-related disorders, such as cancers and viral infections.

### SEQUENCE LISTING

The instant application contains a Sequence Listing which has been submitted electronically in XML format and is hereby incorporated by reference in its entirety. Said XML copy, created on Jun. 30, 2023, is named “51121-027009_Sequence_Listing_6_30_23” and is 456,817 bytes in size.

### BACKGROUND

Disorders can be affected by the BAF complex. BRD9 is a component of the BAF complex. The present invention relates to useful compositions and methods for the treatment of BAF complex-related disorders, such as cancer and infection.

### SUMMARY

Bromodomain-containing protein 9 (BRD9) is a protein encoded by the BRD9 gene on chromosome 5. BRD9 is a component of the BAF (BRG1- or BRM-associated factors) complex, a SWI/SNF ATPase chromatin remodeling complex, and belongs to family IV of the bromodomain-containing proteins. BRD9 is present in several SWI/SNF ATPase chromatin remodeling complexes and is upregulated in multiple cancer cell lines. Accordingly, agents that reduce the levels and/or activity of BRD9 may provide new methods for the treatment of disease and disorders, such as cancer and infection. The inventors have found that depleting BRD9 in cells results in the depletion of the SS18-SSX fusion protein in those cells. The SS18-SSX fusion protein has been detected in more than 95% of synovial sarcoma tumors and is often the only cytogenetic abnormality in synovial sarcoma. Additionally, evidence suggests that the BAF complex is involved in cellular antiviral activities. Thus, agents that degrade BRD9 (e.g., compounds) are useful in the treatment of disorders (e.g., cancers or infections) related to BAF, BRD9, and/or SS18-SSX.

The present disclosure features compounds and methods useful for treating BAF-related disorders (e.g., cancer or infection).

In an aspect, the disclosure features a compound having the structure of Formula I:

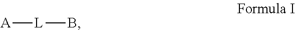

where
A is a BRD9 binding moiety;
B is a degradation moiety; and
L has the structure of Formula II:

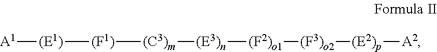

where
A¹ is a bond between the linker and A;
A² is a bond between B and the linker;
each of m, n, o1, o2, and p is, independently, 0 or 1;
each of E¹ and E² is, independently, O, S, NR^{N}, optionally substituted C₁₋₁₀ alkylene, optionally substituted C₂₋₁₀ alkenylene, optionally substituted C₂₋₁₀ alkynylene, optionally substituted C₂-C₁₀ polyethylene glycol, or optionally substituted C₁₋₁₀ heteroalkylene;
E³ is optionally substituted C₁-C₆ alkylene, optionally substituted C₁-C₆ heteroalkylene, O, S, or NRN;
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;
C³ is carbonyl, thiocarbonyl, sulphonyl, or phosphoryl; and
each of F¹, F², and F³ is, independently, optionally substituted C₃-C₁₀ carbocyclylene, optionally substituted C₂₋₁₀ heterocyclylene, optionally substituted C₆-C₁₀ arylene, or optionally substituted C₂-C₉ heteroarylene,
or a pharmaceutically acceptable salt thereof.

In some embodiments, the linker has the structure of Formula IIa:

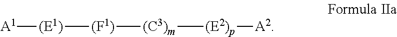

In some embodiments, the linker has the structure of Formula IIb:

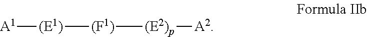

In some embodiments, the linker has the structure of Formula IIc:

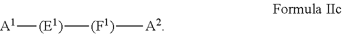

In some embodiments, the linker has the structure of Formula IId:

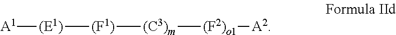

In some embodiments, the linker has the structure of Formula IIe:

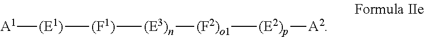

In some embodiments, the linker has the structure of Formula IIf:

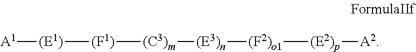

In some embodiments, the linker has the structure of Formula IIg:

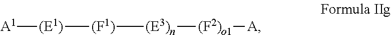

In some embodiments, each of E¹ and E² is, independently, NR^{N}, optionally substituted C₁₋₁₀ alkylene, optionally substituted C₂-C₁₀ polyethylene glycolene, or optionally substituted C₁₋₁₀ heteroalkylene.

In some embodiments, E³ is optionally substituted C₁-C₆ alkylene, O, S, or NR^{N}; In some embodiments, E³ is optionally substituted C₁-C₆ alkylene. In some embodiments, E³ is optionally substituted C₁-C₃ alkylene. In some embodiments, E³ is O, S, or NR^{N}.

In some embodiments, E³ is C₁-C₆ alkylene. In some embodiments, E³ is C₁-C₃ alkylene. In some embodiments, E³ is O.

In some embodiments, E³ is

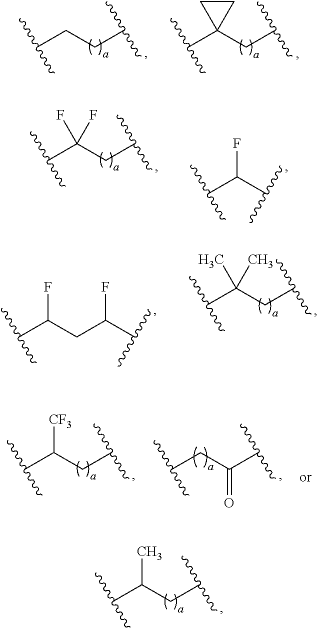

where a is 0, 1, 2, 3, 4, or 5.

In some embodiments, E³ is

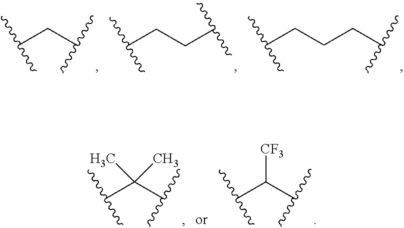

In some embodiments, each R^{N} is, independently, H or optionally substituted C₁₋₄ alkyl.

In some embodiments, each R^{N} is, independently, H or methyl.

In some embodiments, E¹ is

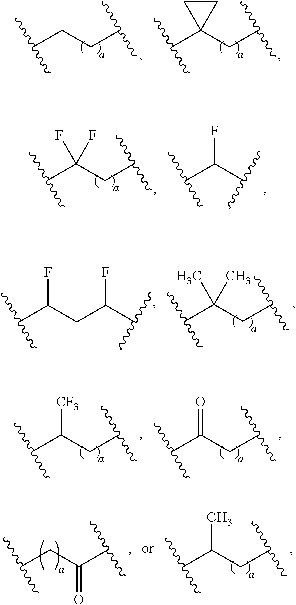

where a is 0, 1, 2, 3, 4, or 5.

In some embodiments, E¹ is

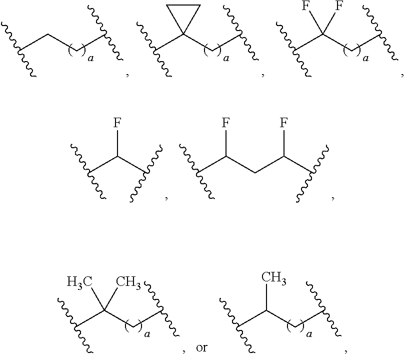

where a is 0, 1, 2, 3, 4, or 5.

In some embodiments, E¹ is

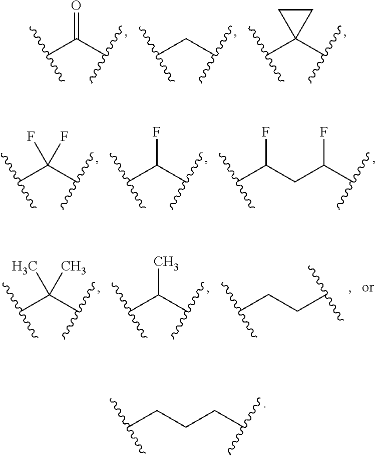

In some embodiments, E¹ is

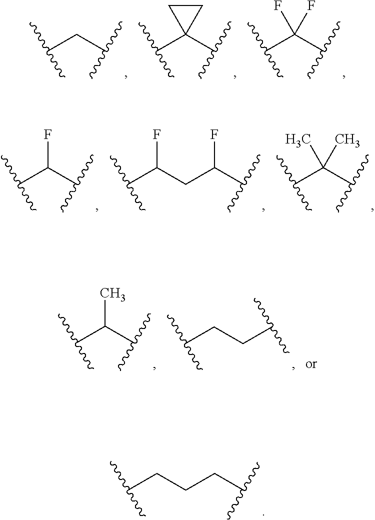

In some embodiments, E¹ is

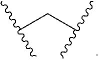 [SMILES: *C*]

In some embodiments, E¹ is

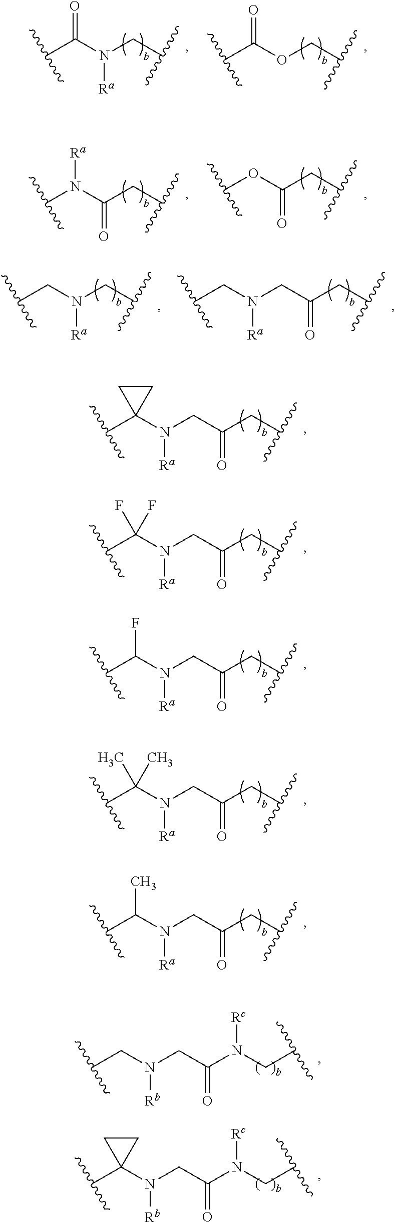

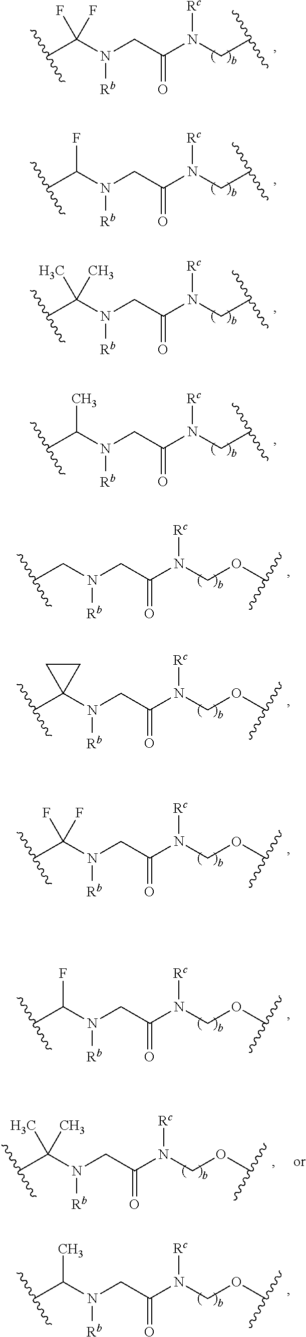

where
b is 0, 1, 2, 3, 4, 5, or 6;
R^{a} is H, optionally substituted C₁-C₆ alkyl, optionally substituted C₁-C₆ heteroalkyl, or optionally substituted C₃-C₆ carbocyclyl;
R^{b} is H, optionally substituted C₁-C₆ alkyl, optionally substituted C₁-C₆ heteroalkyl, or optionally substituted C₃-C₆ carbocyclyl; and
R^{c} is H, optionally substituted C₁-C₆ alkyl, optionally substituted C₁-C₆ heteroalkyl, or optionally substituted C₃-C₆ carbocyclyl.

In some embodiments, E¹ is

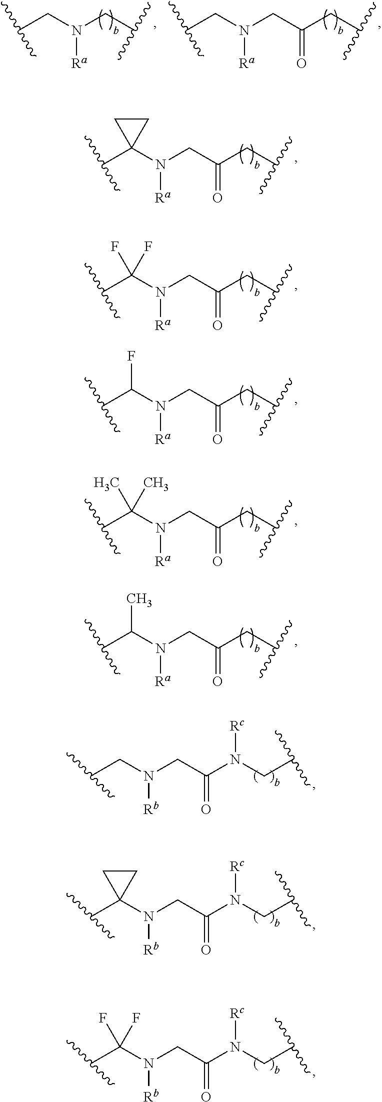

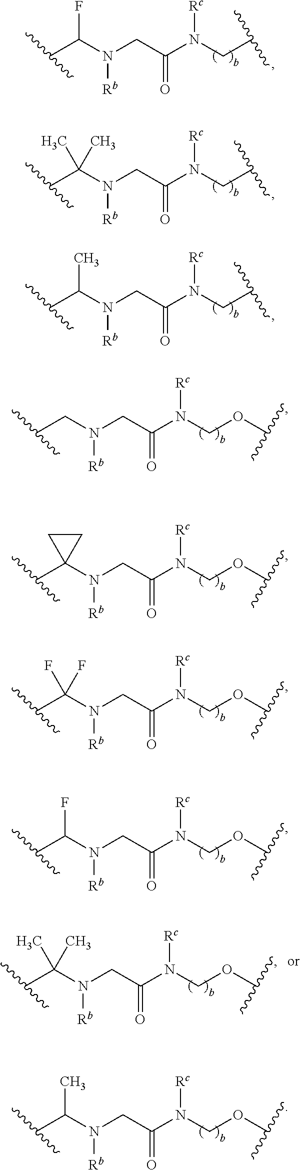

In some embodiments, E¹ is

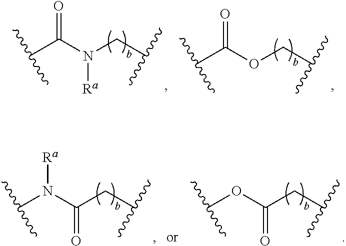

In some embodiments, E¹ is

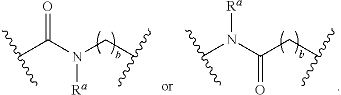

In some embodiments, R^{a} is H or optionally substituted C₁-C₆ alkyl. In some embodiments, R^{b} is H or optionally substituted C₁-C₆ alkyl. In some embodiments, R^{c} is H or optionally substituted C₁-C₆ alkyl.

In some embodiments, R^{a} is H or methyl. In some embodiments, R^{b} is H or methyl. In some embodiments, R^{c} is H or methyl.

In some embodiments, b is 0, 1, 2, or 3. In some embodiments, b is 0. In some embodiments, b is 1. In some embodiments, b is 2. In some embodiments, b is 3.

In some embodiments, E¹ is

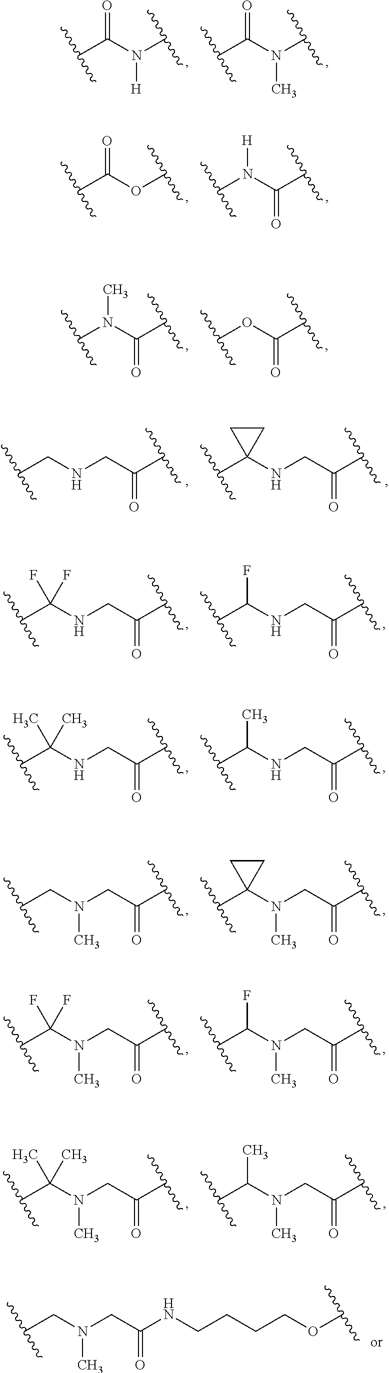

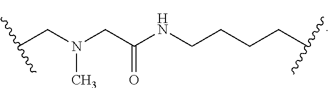

In some embodiments, E¹ is

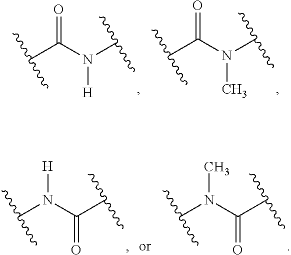

In some embodiments, E¹ is

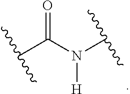 [SMILES: *NC(*)=O]

In some embodiments, E¹ is

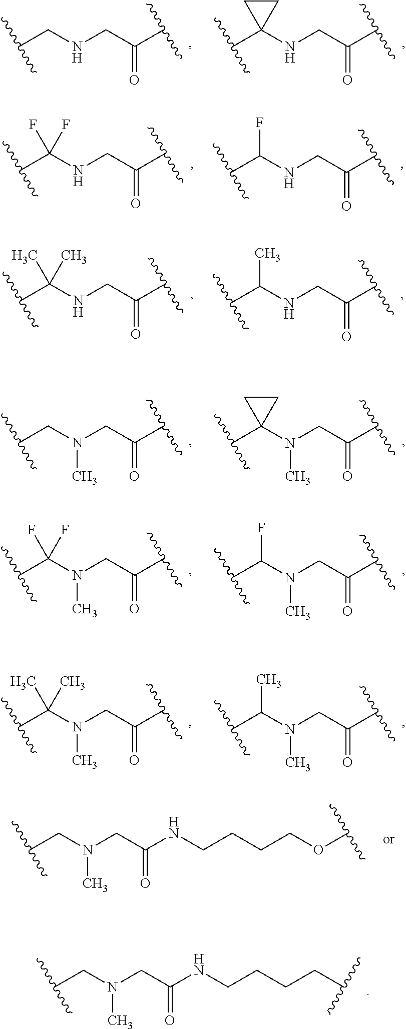

In some embodiments, E¹ is

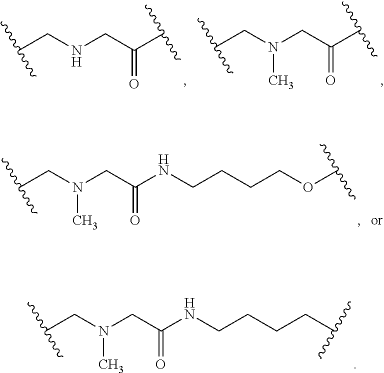

In some embodiments, E¹ is

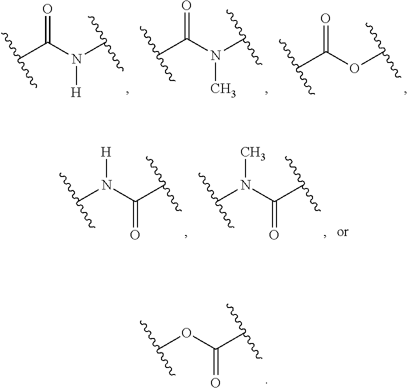

In some embodiments, E² is O, NR^{w},

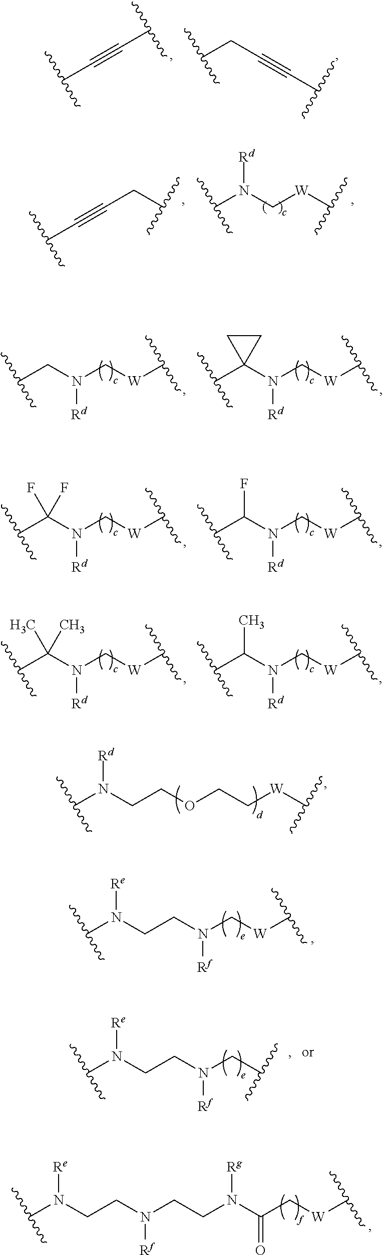

wherein
c is 0, 1, 2, 3, 4, 5, 6, 7, or 8;
d is 0, 1, 2, or 3;
e is 0, 1, 2, 3, 4, 5, or 6;
f is 0, 1, 2, 3, or 4;
R^{d} is H, optionally substituted C₁-C₆ alkyl, optionally substituted C₁-C₆ heteroalkyl, or optionally substituted C₃-C₆ carbocyclyl;
R^{e} is H, optionally substituted C₁-C₆ alkyl, optionally substituted C₁-C₆ heteroalkyl, or optionally substituted C₃-C₆ carbocyclyl;
R^{f} is H, optionally substituted C₁-C₆ alkyl, optionally substituted C₁-C₆ heteroalkyl, or optionally substituted C₃-C₆ carbocyclyl;
R^{g} is H, optionally substituted C₁-C₆ alkyl, optionally substituted C₁-C₆ heteroalkyl, or optionally substituted C₃-C₆ carbocyclyl; and
W is O or NRW, wherein R^{w} is H or optionally substituted C₁-C₆ alkyl.

In some embodiments, E² is O, NR^{w},

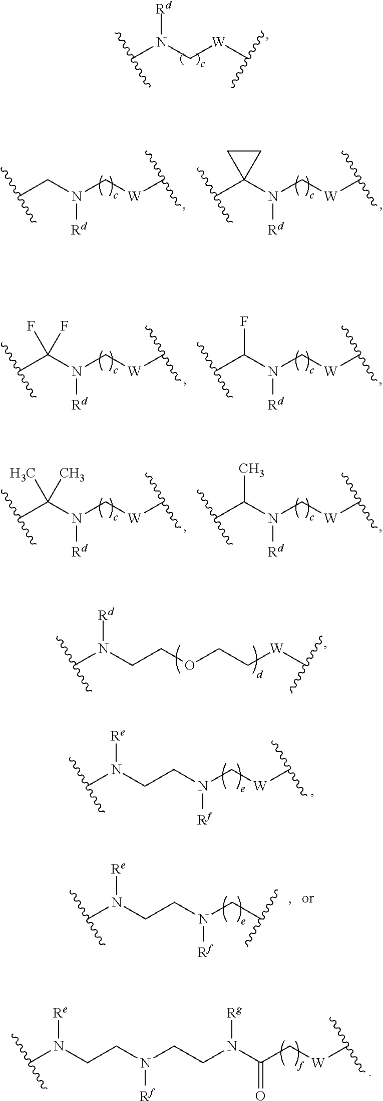

In some embodiments, R^{d} is H or optionally substituted C₁-C₆ alkyl. In some embodiments, R^{e} is H or optionally substituted C₁-C₆ alkyl. In some embodiments, R^{f} is H or optionally substituted C₁-C₆ alkyl. In some embodiments, R^{g} is H or optionally substituted C₁-C₆ alkyl. In some embodiments, R^{w} is H or optionally substituted C₁-C₆ alkyl.

In some embodiments, R^{d} is H or methyl. In some embodiments, R^{e} is H or methyl. In some embodiments, R^{f} is H or methyl. In some embodiments, R^{g} is H or methyl. In some embodiments, R^{w} is H or methyl.

In some embodiments, E² is

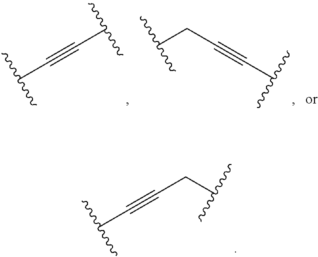

In some embodiments, E² is O,

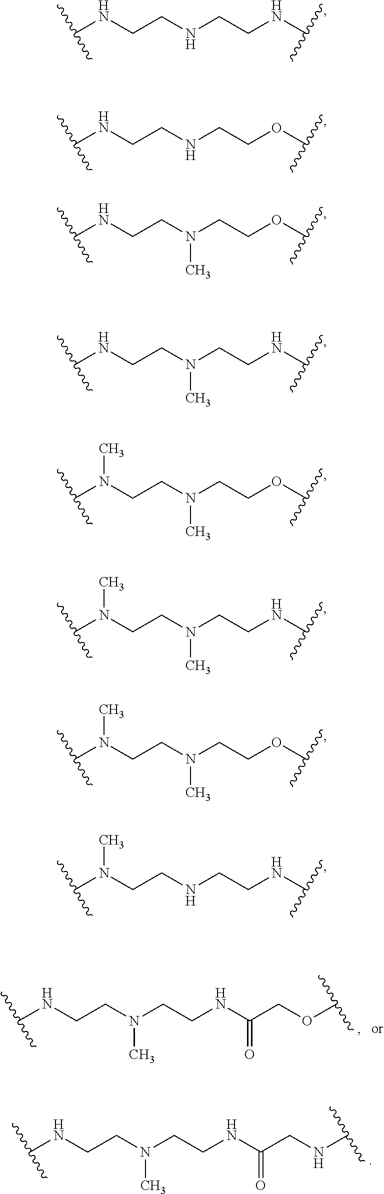

In some embodiments, each of F¹, F², or F³ is, independently, optionally substituted C₃-C₁₀ carbocyclylene.

In some embodiments, the C₃-C₁₀ carbocyclylene is monocyclic. In some embodiments, the C₃-C₁₀ carbocyclylene is polycyclic.

In some embodiments, the C₃-C₁₀ carbocyclylene is bicyclic.

In some embodiments, the C₃-C₁₀ carbocyclylene is bridged. In some embodiments, the C₃-C₁₀ carbocyclylene is fused. In some embodiments, the C₃-C₁₀ carbocyclylene is spirocyclic.

In some embodiments, the C₃-C₁₀ carbocyclylene is

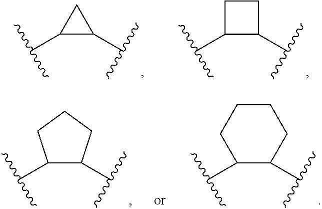 [SMILES: *C1CC1* ; *C1CCC1* ; *C1CCCC1* ; *C1CCCCC1*]

In some embodiments, F² is

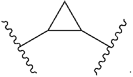 [SMILES: *C1CC1*]

In some embodiments, the C₃-C₁₀ carbocyclylene is

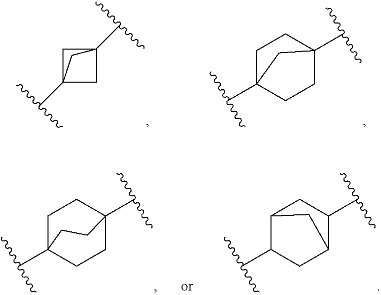 [SMILES: *C12CC(*)(C1)C2 ; *C12CCC(*)(CC1)C2 ; *C12CCC(*)(CC1)CC2 ; *C1CC2CC1CC2*]

In some embodiments, F¹ is

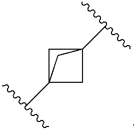 [SMILES: *C12CC(*)(C1)C2]

In some embodiments, each of F¹, F², or F³ is, independently, optionally substituted C₂-C₉ heterocyclylene.

In some embodiments, the C₂-C₉ heterocyclylene is monocyclic. In some embodiments, the C₂-C₉ heterocyclylene is polycyclic.

In some embodiments, the C₂-C₉ heterocyclylene is bicyclic.

In some embodiments, the C₂-C₉ heterocyclylene is bridged. In some embodiments, the C₂-C₉ heterocyclylene is fused. In some embodiments, the C₂-C₉ heterocyclylene is spirocyclic.

In some embodiments, the C₂-C₉ heterocyclylene includes a quaternary amine.

In some embodiments, the C₂-C₉ heterocyclylene is

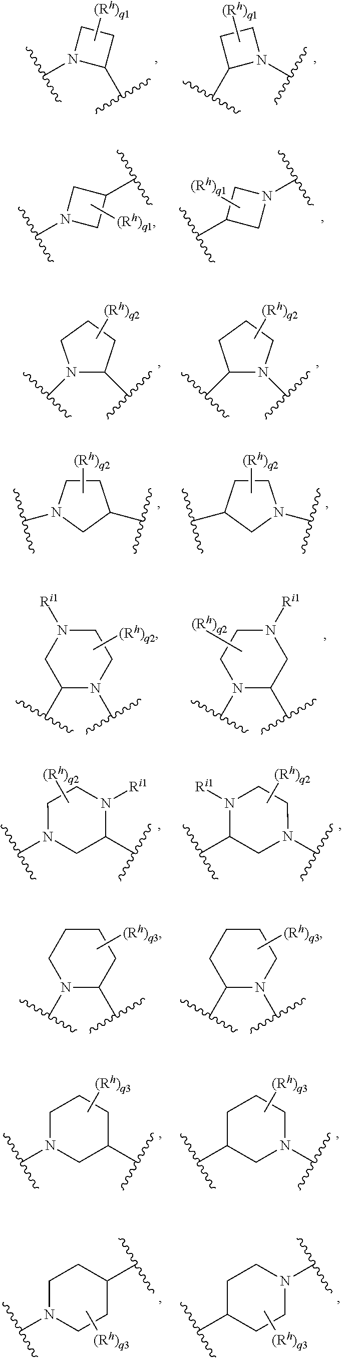

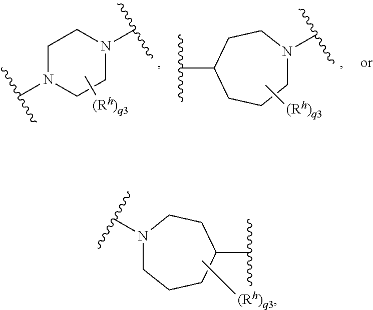

where
q1 is 0, 1, 2, 3, or 4;
q2 is 0, 1, 2, 3, 4, 5, or 6;
q3 is 0, 1, 2, 3, 4, 5, 6, 7, or 8;
each R^{h} is, independently, ²H, halogen, optionally substituted C₁-C₆ alkyl, ORⁱ², or NRⁱ³Rⁱ⁴; or two R^{h} groups, together with the carbon atom to which each is attached, combine to form optionally substituted C₃-C₁₀ carbocyclyl or optionally substituted C₂-C₉ heterocyclyl; or two R^{h} groups, together with the carbon atoms to which each is attached, combine to form optionally substituted C₃-C₁₀ carbocyclyl or optionally substituted C₂-C₉ heterocyclyl;
Rⁱ¹ is H or optionally substituted C₁-C₆ alkyl;
Rⁱ² is H, optionally substituted C₁-C₆ alkyl, optionally substituted C₁-C₆ heteroalkyl, or optionally substituted C₃-C₆ carbocyclyl;
Rⁱ³ is H or optionally substituted C₁-C₆ alkyl; and
Rⁱ⁴ is H or optionally substituted C₁-C₆ alkyl.

In some embodiments, each R^{h} is, independently, halogen, optionally substituted C₁-C₆ alkyl, ORⁱ², or NRⁱ³Rⁱ⁴. In some embodiments, Rⁱ¹ is H or optionally substituted C₁-C₆ alkyl. In some embodiments, Rⁱ² is H or optionally substituted C₁-C₆ alkyl. In some embodiments, Rⁱ³ is H or optionally substituted C₁-C₆ alkyl. In some embodiments, Rⁱ⁴ is H or optionally substituted C₁-C₆ alkyl.

In some embodiments, the C₂-C₉ heterocyclylene is

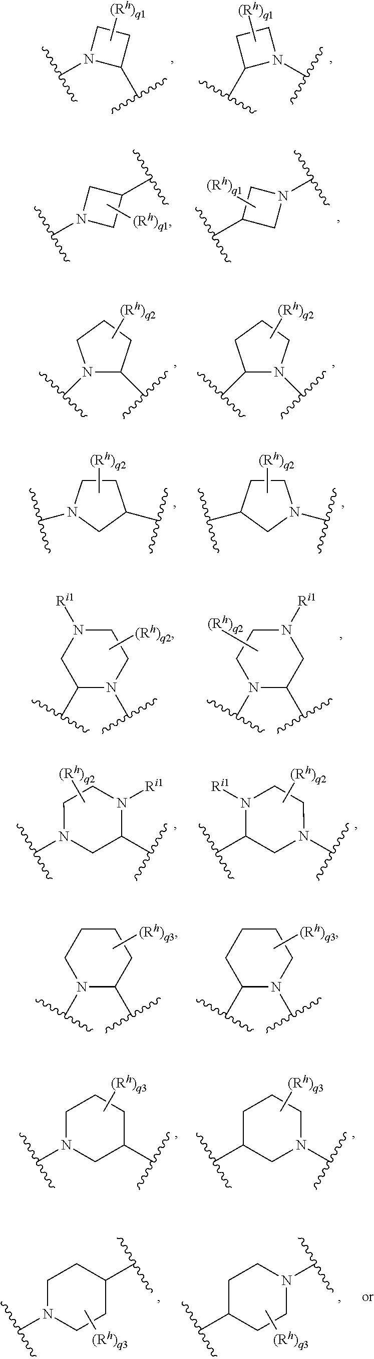

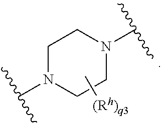

In some embodiments, each R^{h} is, independently, halogen, optionally substituted C₁-C₆ alkyl, ORⁱ², or NRⁱ³Rⁱ⁴. In some embodiments, each R^{h} is, independently, halogen, optionally substituted C₁-C₆ alkyl, or NRⁱ³Rⁱ⁴.

In some embodiments, each R^{h} is, independently, ²H, halogen, cyano, optionally substituted C₁-C₆ alkyl, ORⁱ², or NRⁱ³Rⁱ⁴. In some embodiments, two R^{h} groups, together with the carbon atom to which each is attached, combine to form optionally substituted C₃-C₁₀ carbocyclyl or optionally substituted C₂-C₉ heterocyclyl. In some embodiments, two R^{h} groups, together with the carbon atoms to which each is attached, combine to form optionally substituted C₃-C₁₀ carbocyclyl or optionally substituted C₂-C₉ heterocyclyl.

In some embodiments, each R^{h} is, independently, ²H, F, methyl,

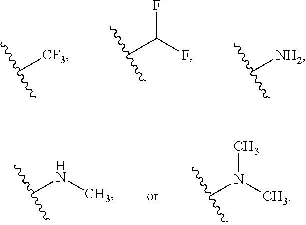

In some embodiments, each R^{h} is, independently, F, methyl, or NRⁱ³Rⁱ⁴.

In some embodiments, q1 is 0, 1, or 2. In some embodiments, q1 is 0. In some embodiments, q1 is 1. In some embodiments, q1 is 2.

In some embodiments, q2 is 0, 1, or 2. In some embodiments, q2 is 0. In some embodiments, q2 is 1. In some embodiments, q2 is 2.

In some embodiments, q3 is 0, 1, or 2. In some embodiments, q3 is 0. In some embodiments, q3 is 1. In some embodiments, q3 is 2.

In some embodiments, the C₂-C₉ heterocyclylene is

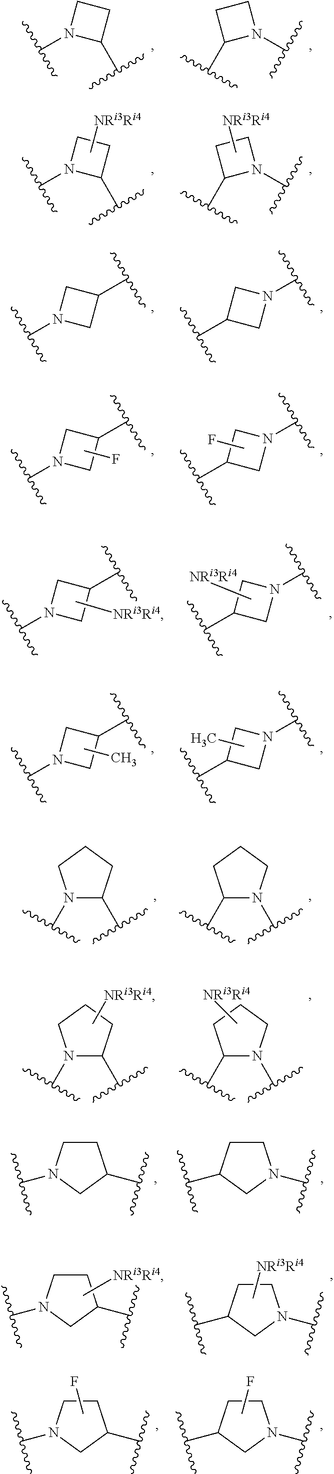

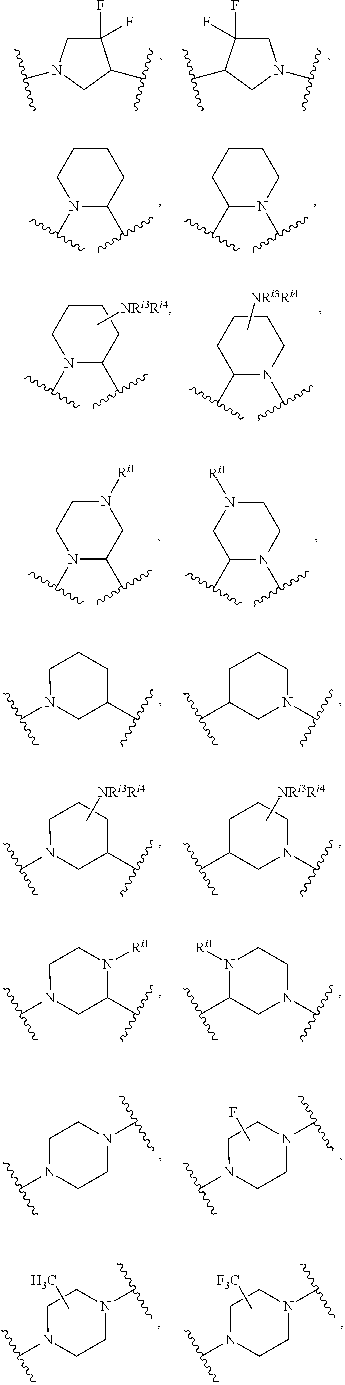

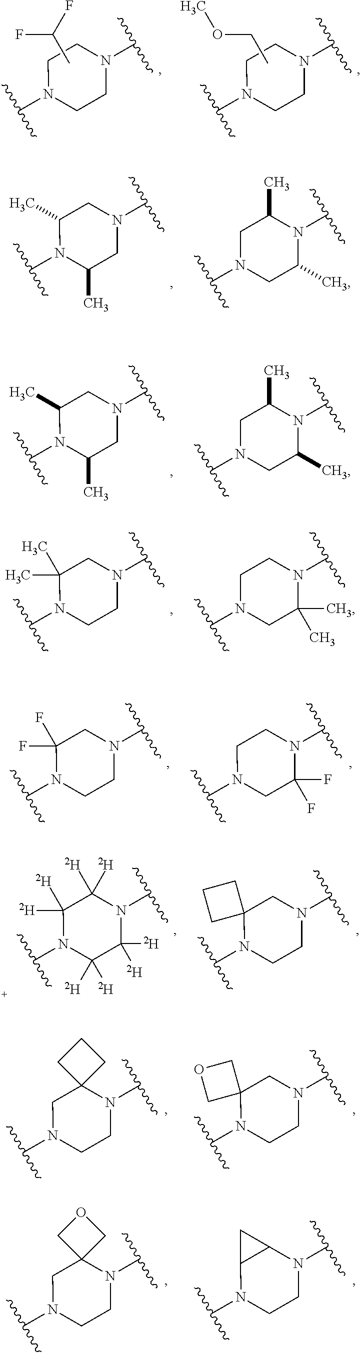

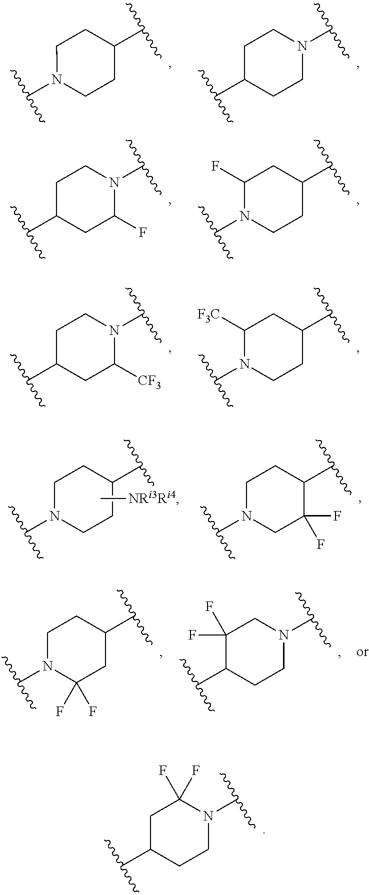

In some embodiments, the C₂-C₉ heterocyclylene is

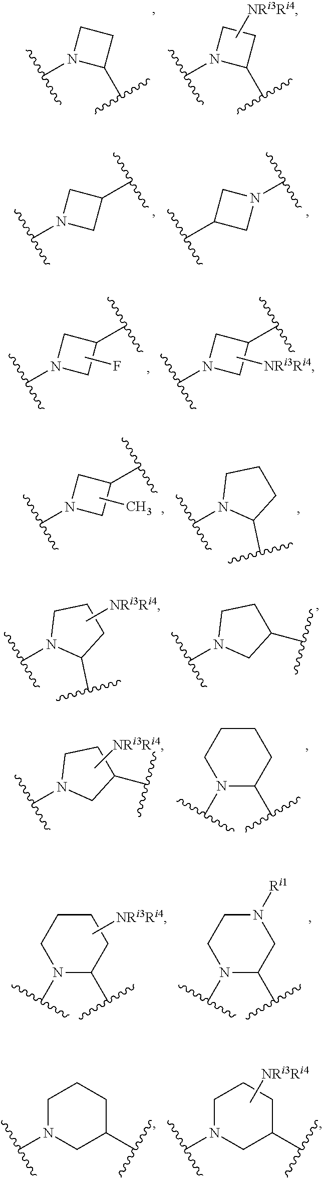

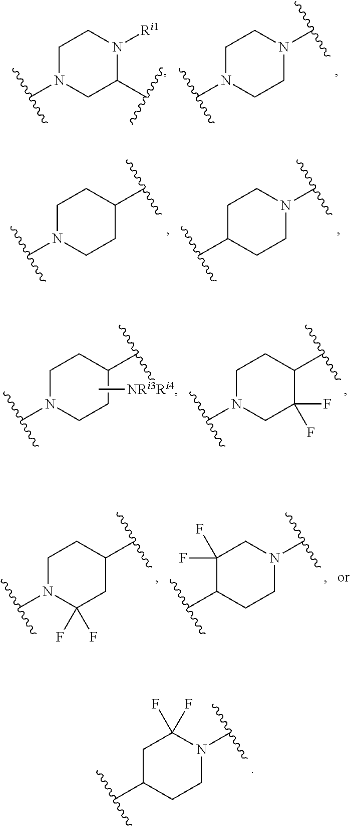

In some embodiments, the C₂-C₉ heterocyclylene is

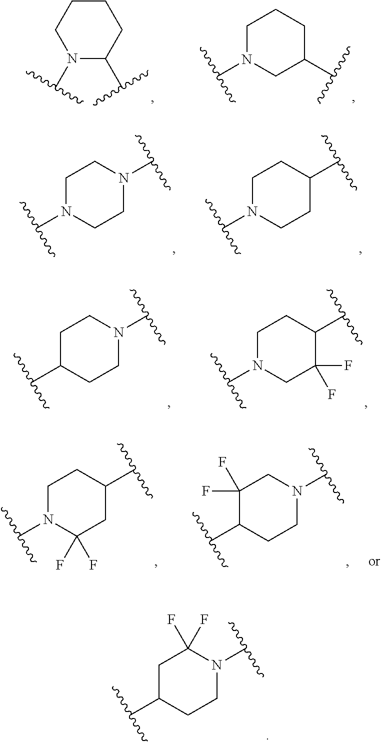

In some embodiments, the C₂-C₉ heterocyclylene is

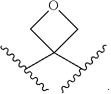 [SMILES: *C1(*)COC1]

In some embodiments, F¹ is

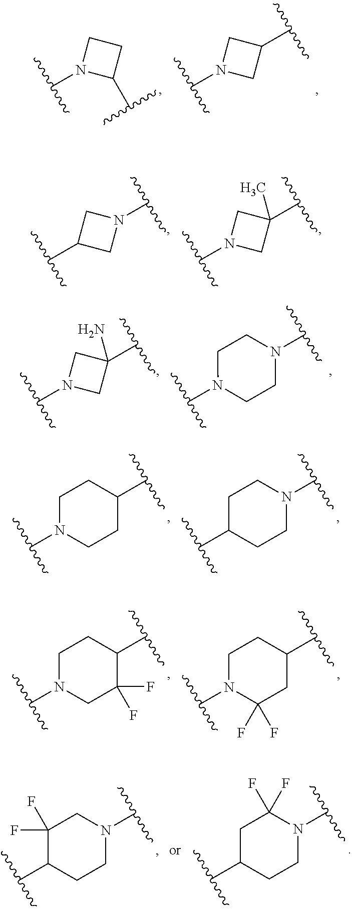

In some embodiments, F¹ is

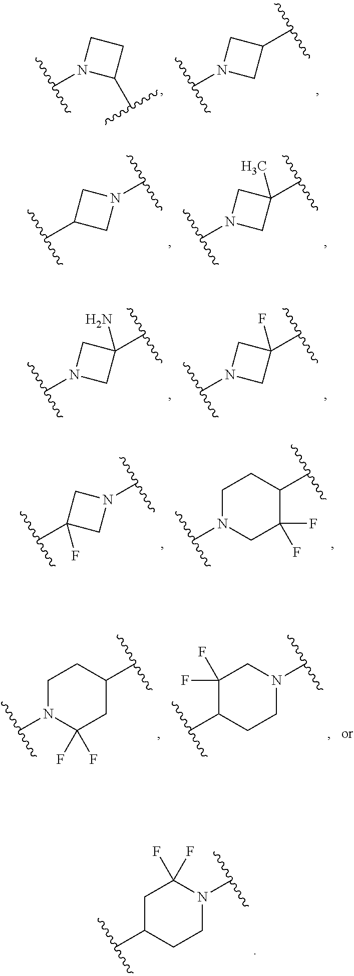

In some embodiments, F¹ is

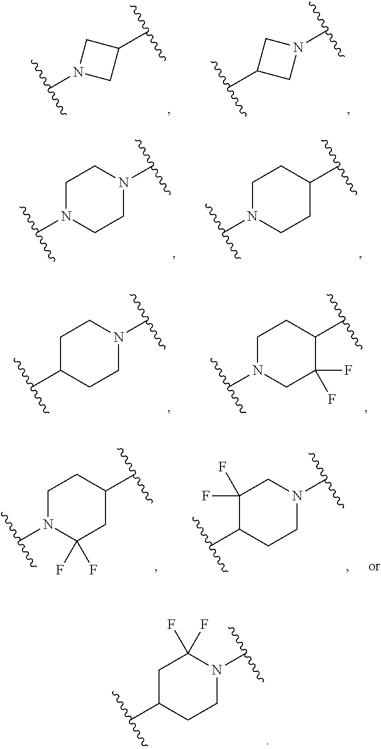

In some embodiments, F² is

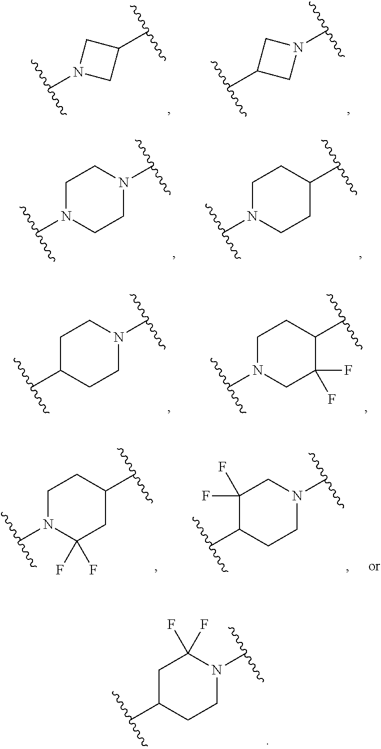

In some embodiments, F² is

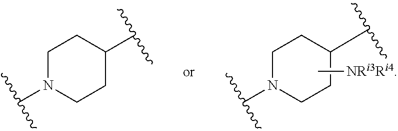

In some embodiments, F³ is

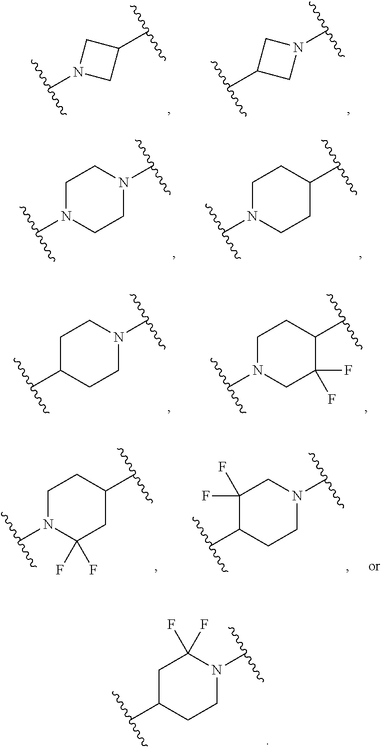

In some embodiments, F³ is

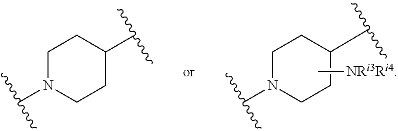

In some embodiments, Rⁱ¹ is H or methyl. In some embodiments, Rⁱ² is H or methyl. In some embodiments, Rⁱ³ is H or methyl. In some embodiments, Rⁱ⁴ is H or methyl.

In some embodiments, the C₂-C₉ heterocyclylene is

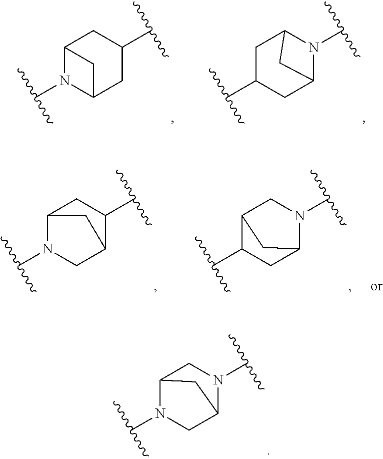 [SMILES: *C1CC2CC(C1)N2* ; *C1CC2CC(C1)N2* ; *C1CC2CC1CN2* ; *C1CC2CC1CN2* ; *N1CC2CC1CN2*]

In some embodiments, the C₂-C₉ heterocyclylene is

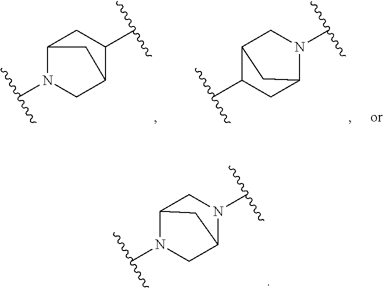 [SMILES: *C1CC2CC1CN2* ; *C1CC2CC1CN2* ; *N1CC2CC1CN2*]

In some embodiments, the C₂-C₉ heterocyclylene is

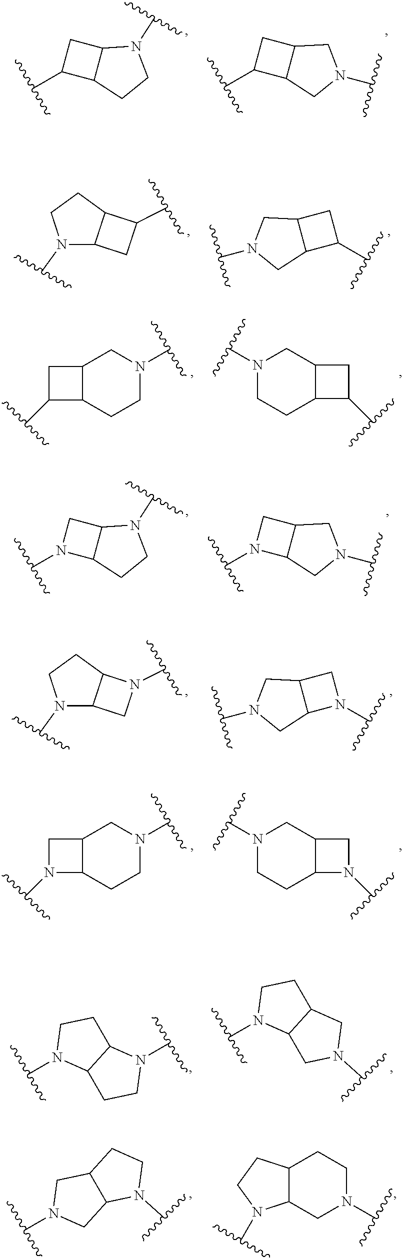

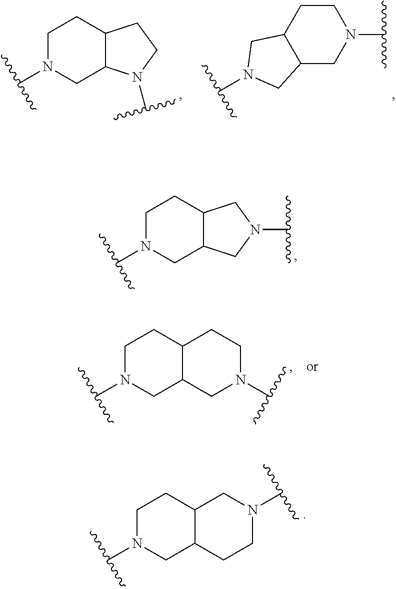

In some embodiments, the C₂-C₉ heterocyclylene is

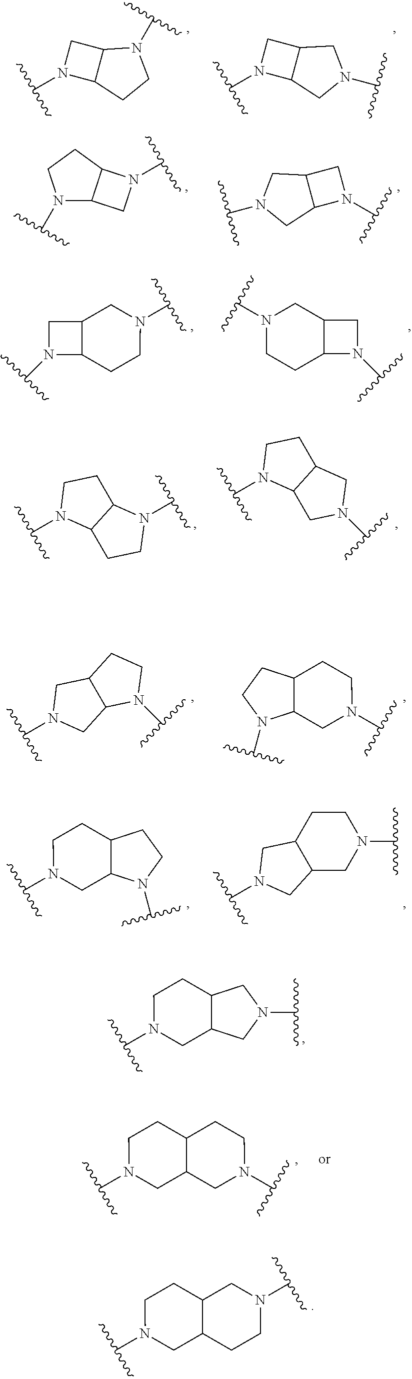

In some embodiments, the C₂-C₉ heterocyclylene is

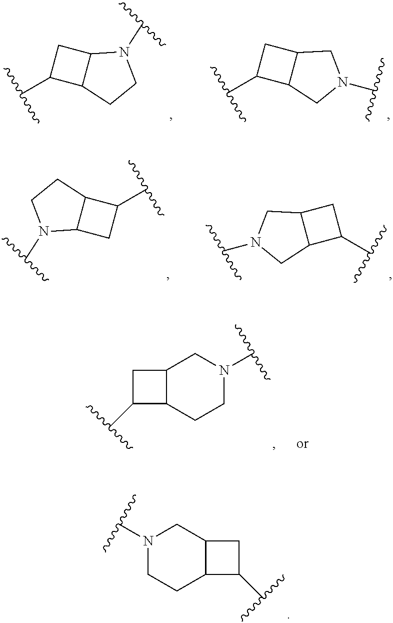 [SMILES: *C1CC2C1CCN2* ; *C1CC2CN(*)CC12 ; *C1CC2C1CCN2* ; *C1CC2CN(*)CC12 ; *C1CC2CN(*)CCC12 ; *C1CC2CN(*)CCC12]

In some embodiments, F¹ is

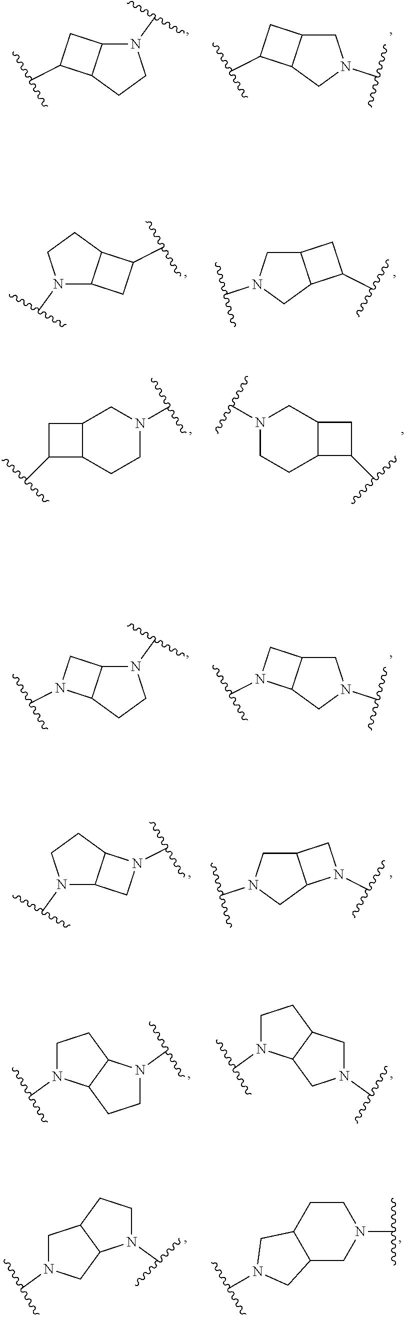

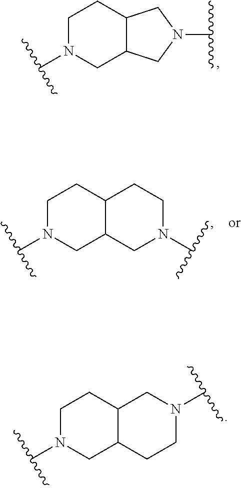 [SMILES: *N1CCC2CN(*)CC2C1 ; *N1CCC2CCN(*)CC2C1 ; *N1CCC2CN(*)CCC2C1]

In some embodiments, F¹ is

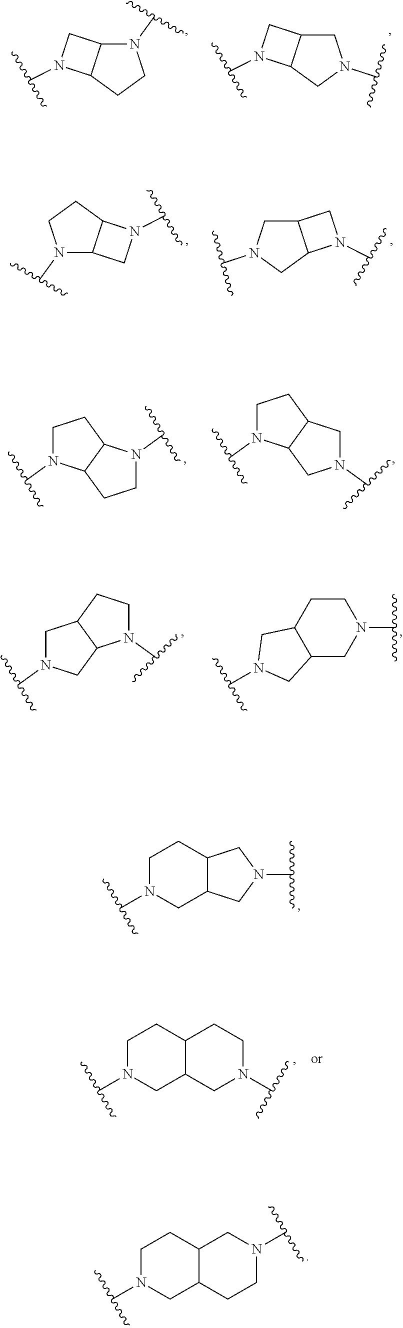

In some embodiments, F¹ is

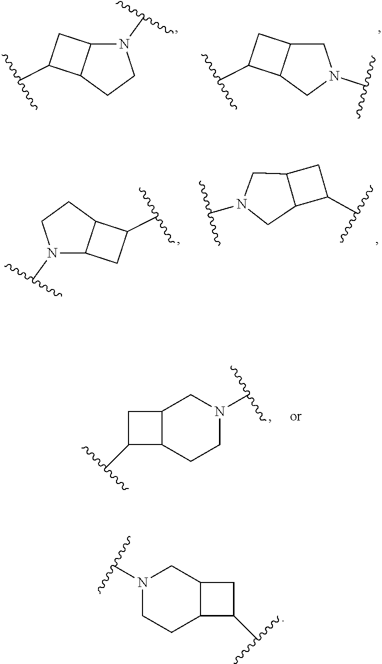 [SMILES: *C1CC2C1CCN2* ; *C1CC2CN(*)CC12 ; *C1CC2C1CCN2* ; *C1CC2CN(*)CC12 ; *C1CC2CN(*)CCC12 ; *C1CC2CN(*)CCC12]

In some embodiments, F² is

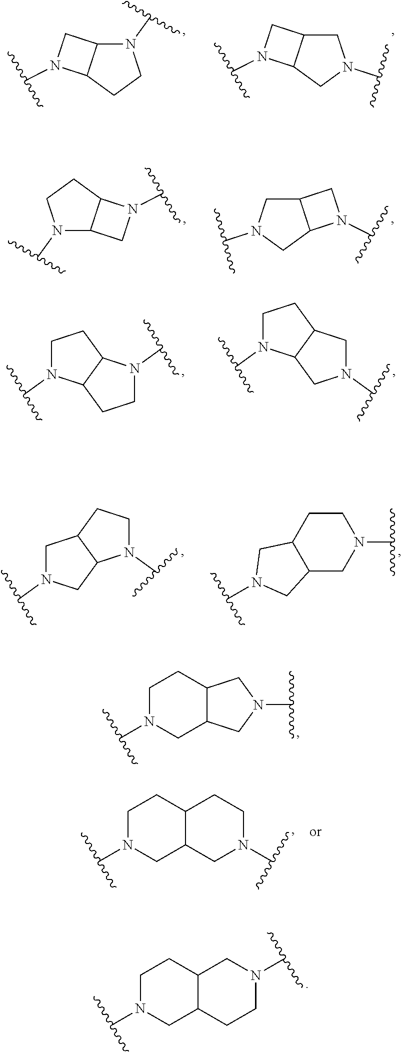

In some embodiments, the C₂-C₉ heterocyclyl is

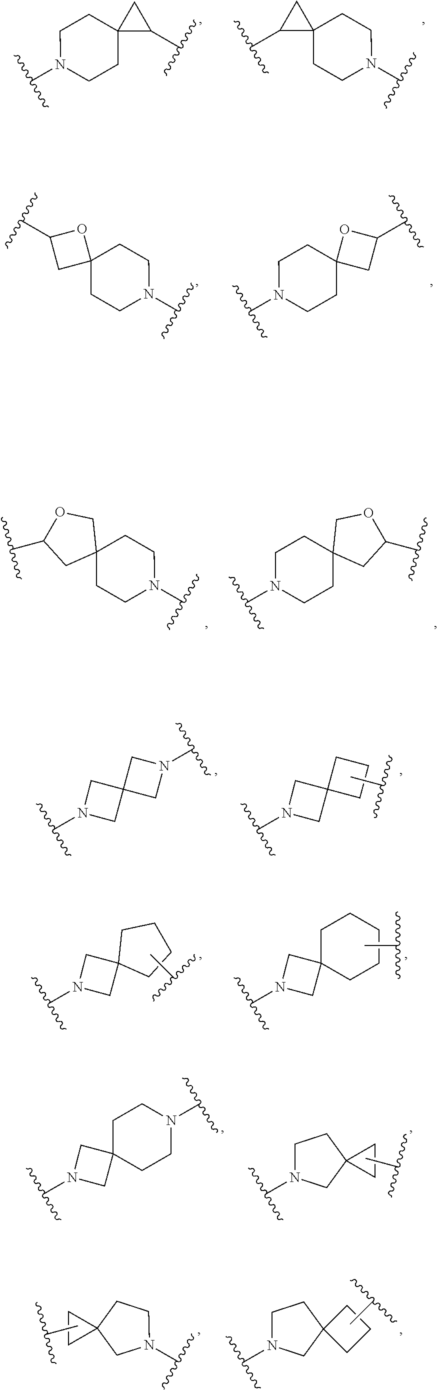

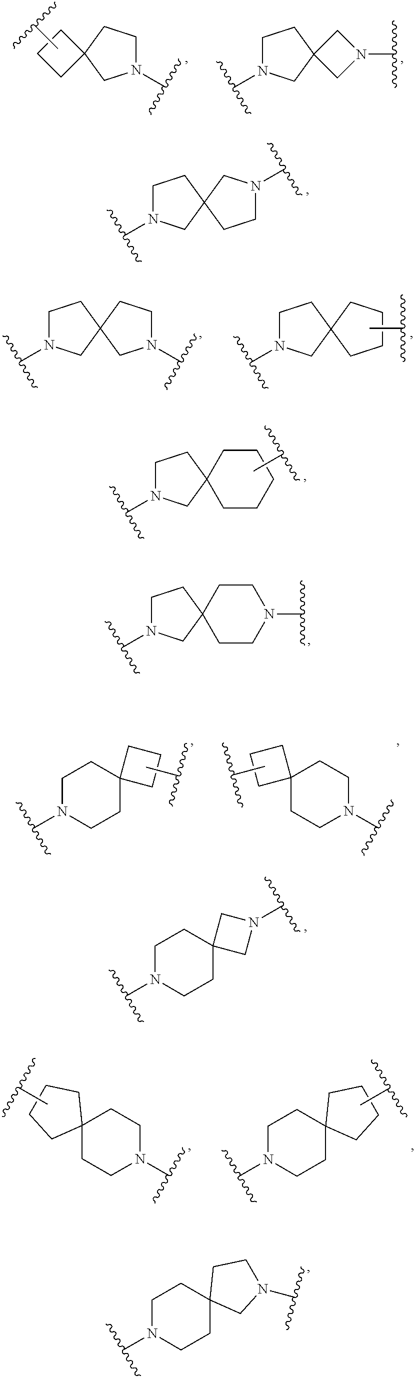

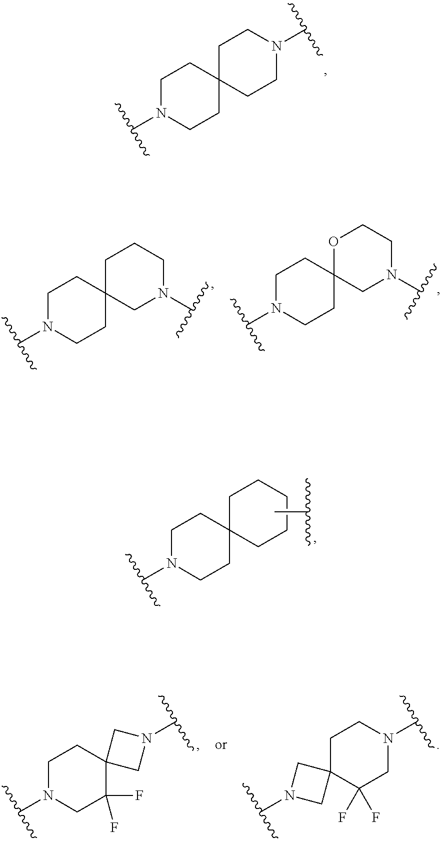

In some embodiments, the C₂-C₉ heterocyclyl is

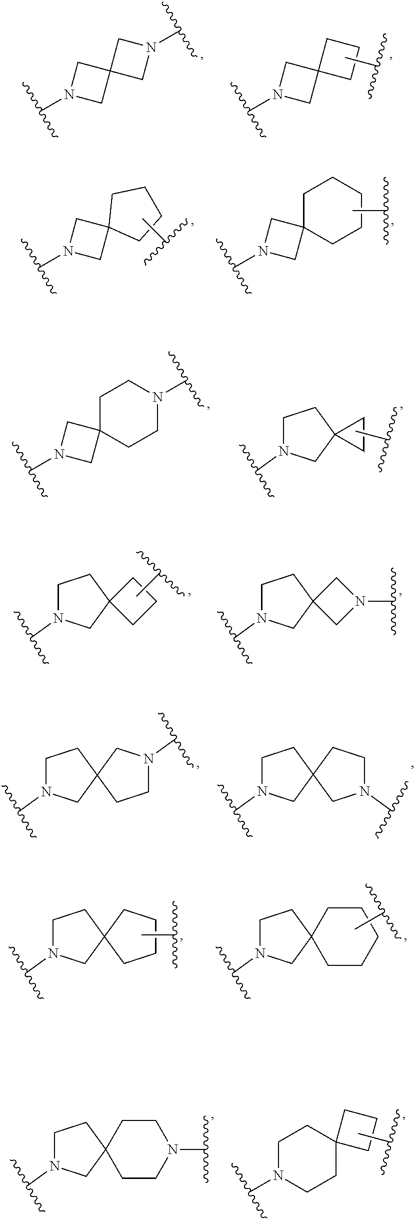

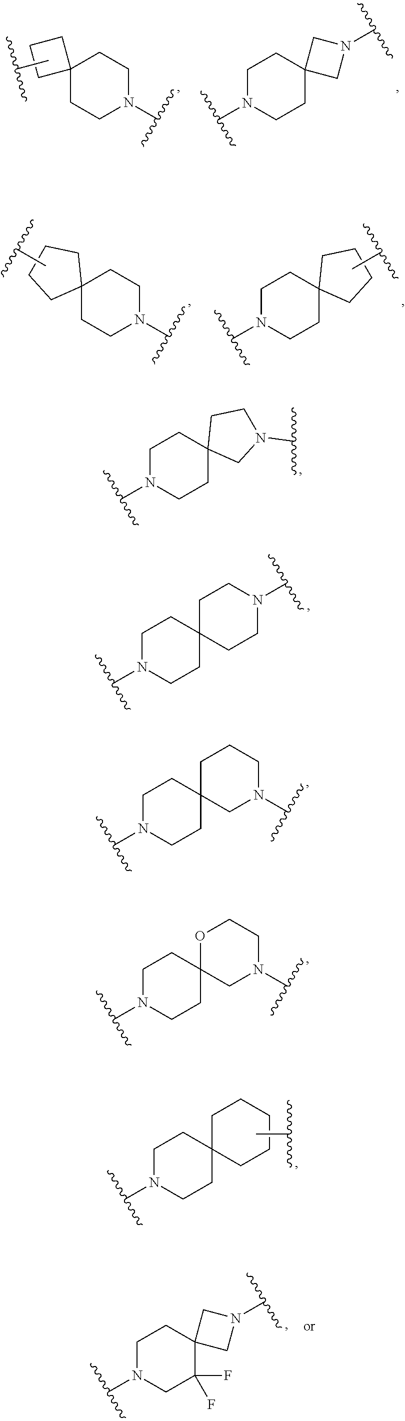

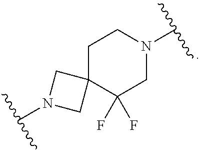 [SMILES: *N1CCC2(CN(*)C2)C(F)(F)C1]

In some embodiments, the C₂-C₉ heterocyclyl is

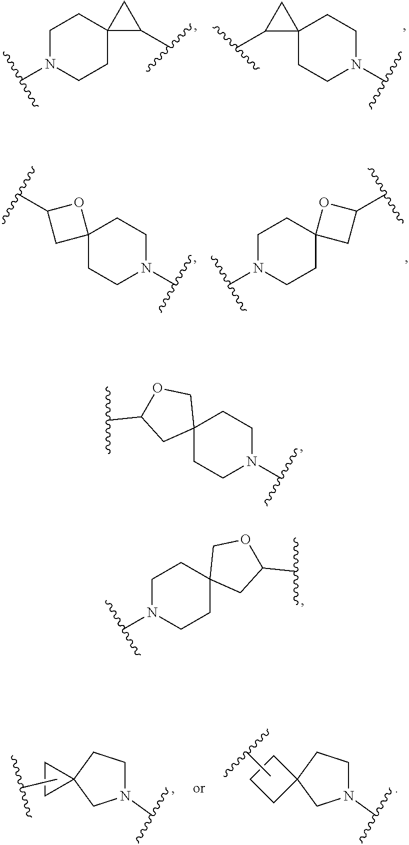

In some embodiments, the C₂-C₉ heterocyclyl is

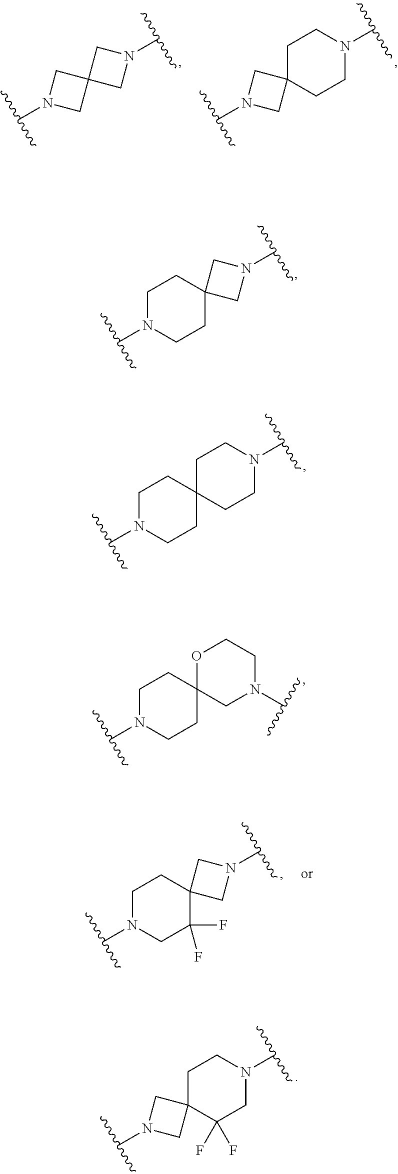

In some embodiments, F¹ is

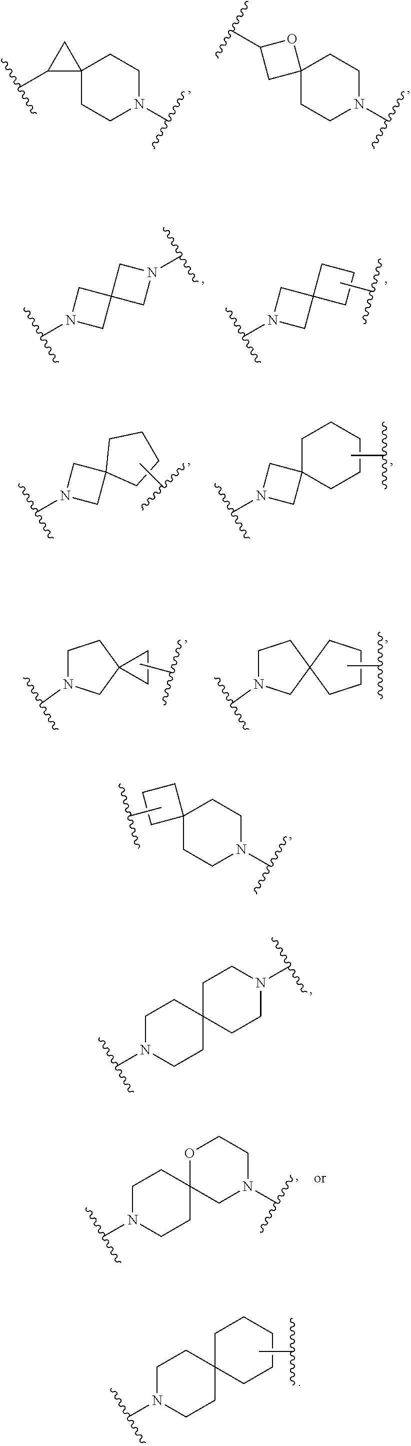

In some embodiments, F¹ is

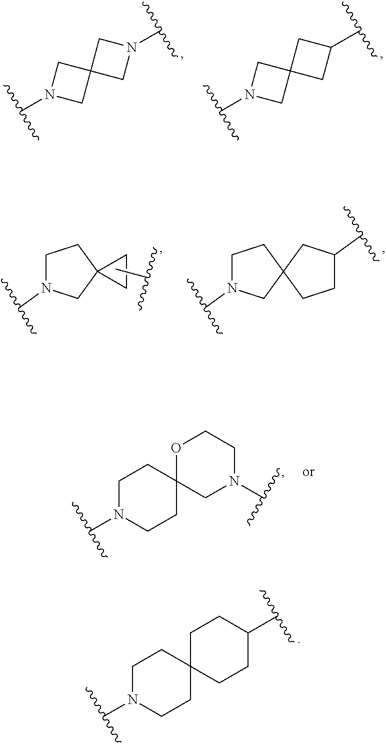

In some embodiments, F¹ is,

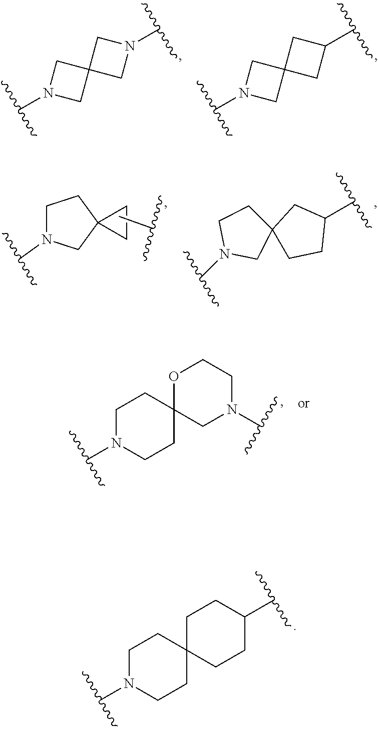

In some embodiments, F¹ is

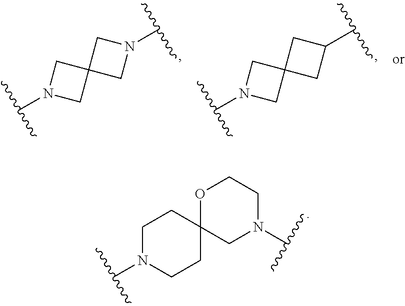 [SMILES: *N1CC2(C1)CN(*)C2 ; *C1CC2(C1)CN(*)C2 ; *N1CCC2(CC1)CN(*)CCO2]

In some embodiments, F¹ is

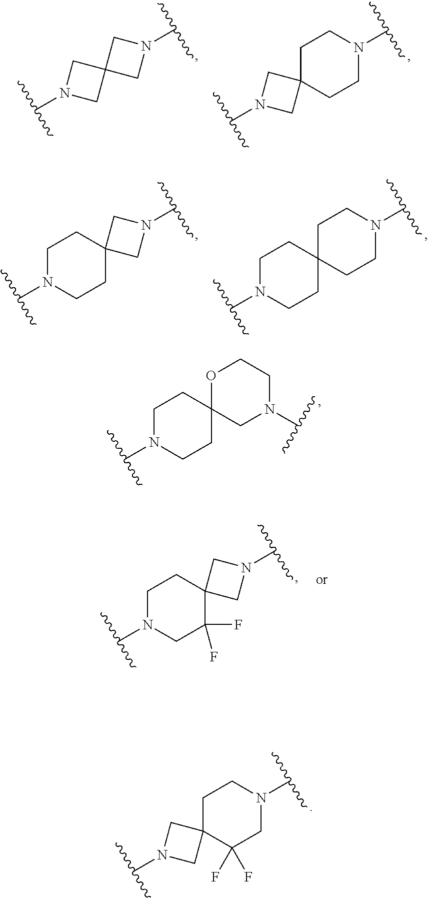

In some embodiments, F² is

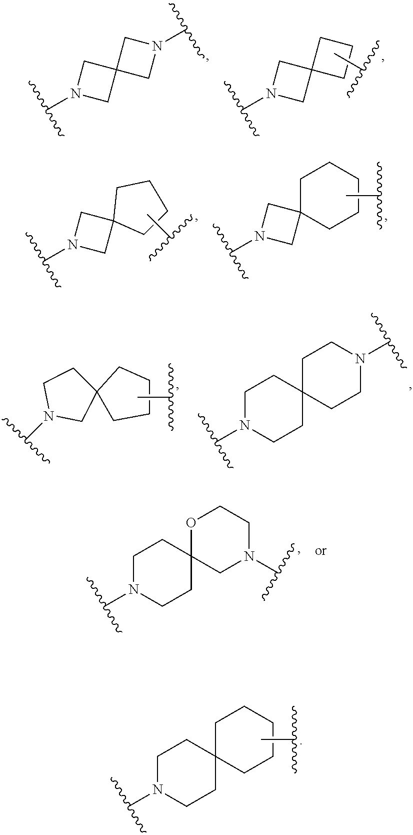

In some embodiments, F² is

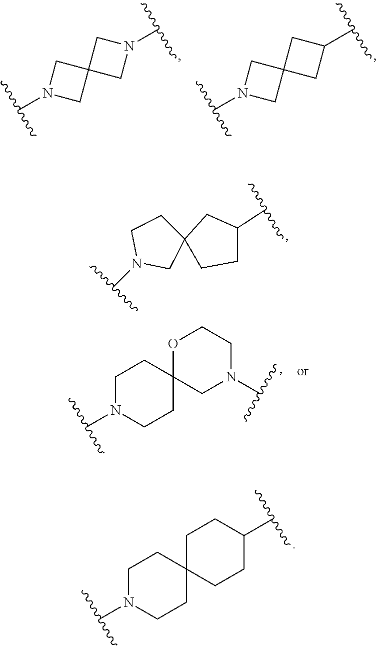 [SMILES: *N1CC2(C1)CN(*)C2 ; *C1CC2(C1)CN(*)C2 ; *C1CCC2(CCN(*)C2)C1 ; *N1CCC2(CC1)CN(*)CCO2 ; *C1CCC2(CC1)CCN(*)CC2]

In some embodiments, F² is

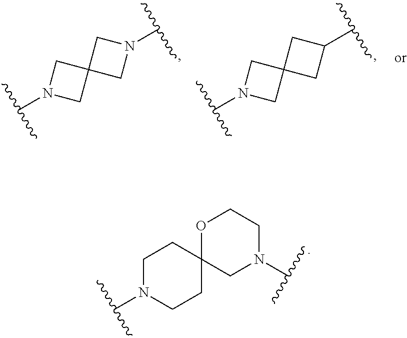 [SMILES: *N1CC2(C1)CN(*)C2 ; *C1CC2(C1)CN(*)C2 ; *N1CCC2(CC1)CN(*)CCO2]

In some embodiments, F² is

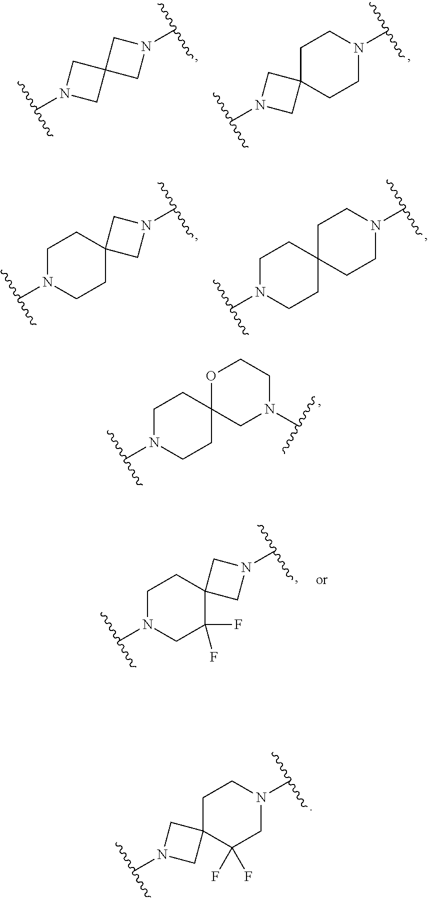

In some embodiments, F³ is

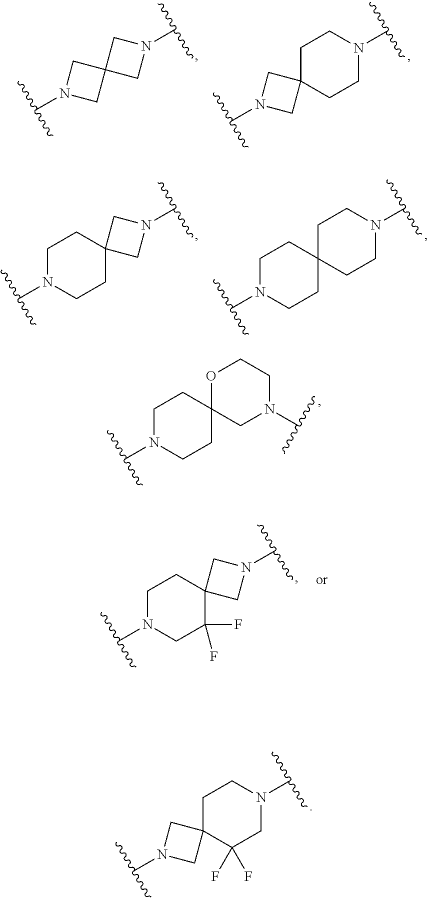

In some embodiments, each of F¹, F², or F³ is, independently, optionally substituted C₆-C₁₀ arylene.

In some embodiments, the C₆-C₁₀ arylene is

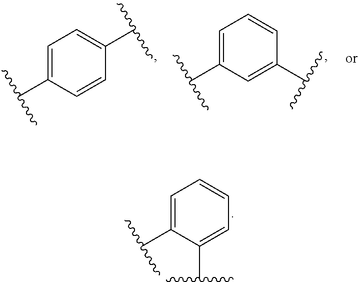 [SMILES: *c1ccc(*)cc1 ; *c1cccc(*)c1 ; *c1ccccc1*]

In some embodiments, each of F¹, F², or F³ is, independently, optionally substituted C₂-C₉ heteroarylene.

In some embodiments, the C₂-C₉ heteroarylene is

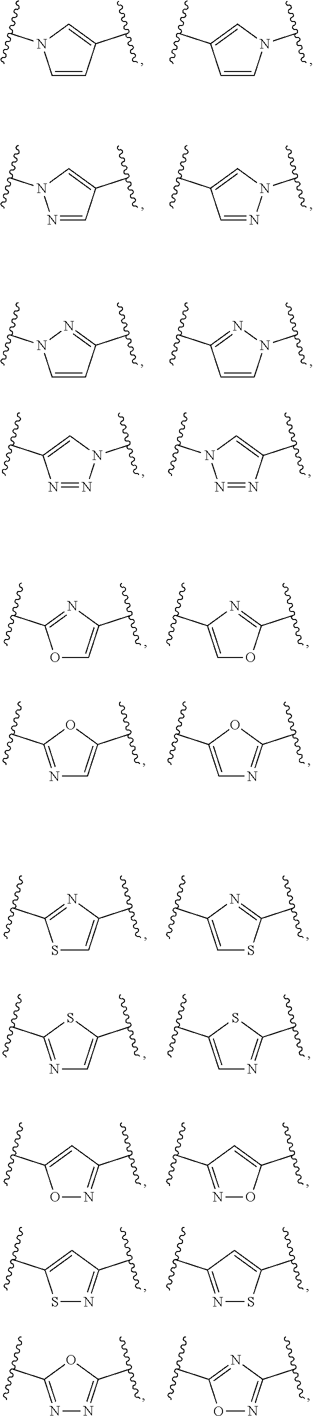

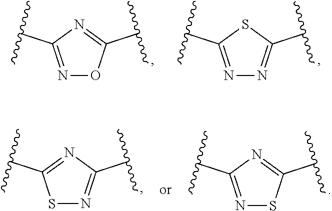 [SMILES: *c1noc(*)n1 ; *c1nnc(*)s1 ; *c1nsc(*)n1 ; *c1nsc(*)n1]

In some embodiments, F² is

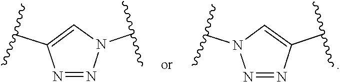 [SMILES: *c1cn(*)nn1 ; *c1cn(*)nn1]

In some embodiments, F² is

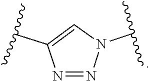 [SMILES: *c1cn(*)nn1]

In some embodiments, C³ is

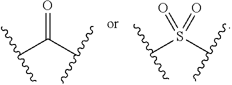

In some embodiments, C³ is

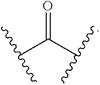 [SMILES: *C(*)=O]

In some embodiments, m is 1. In some embodiments, p is 1.

In some embodiments, the linker has the structure of

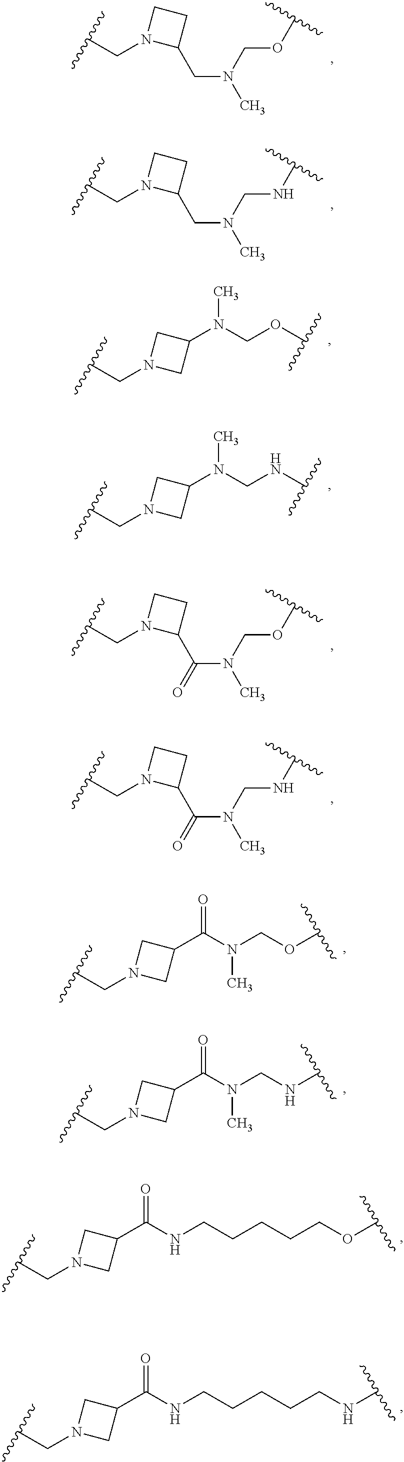

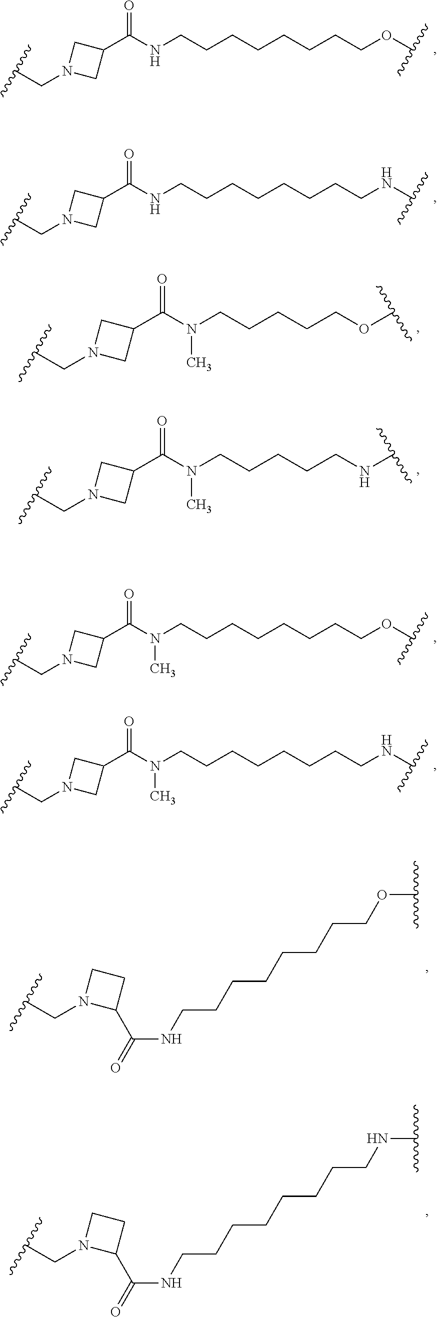

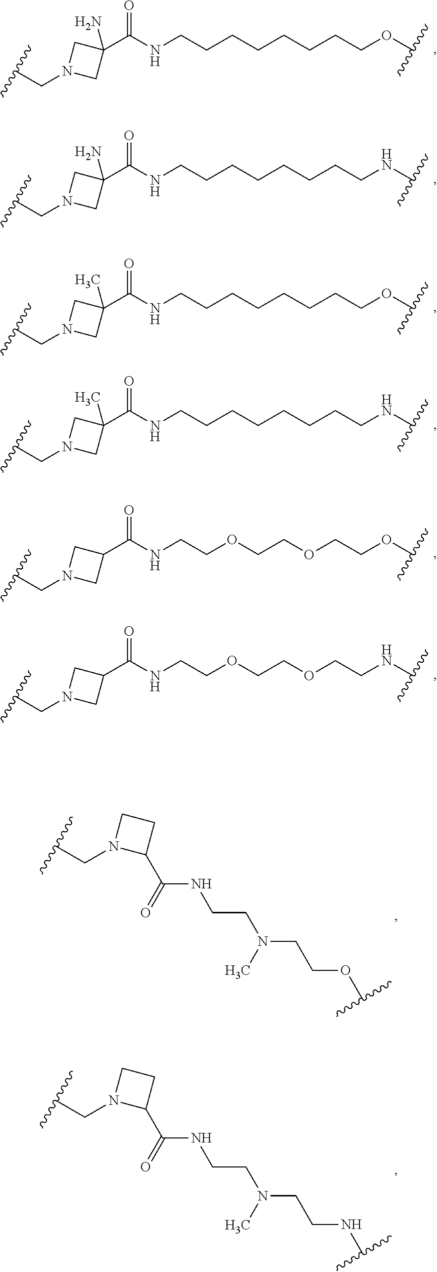

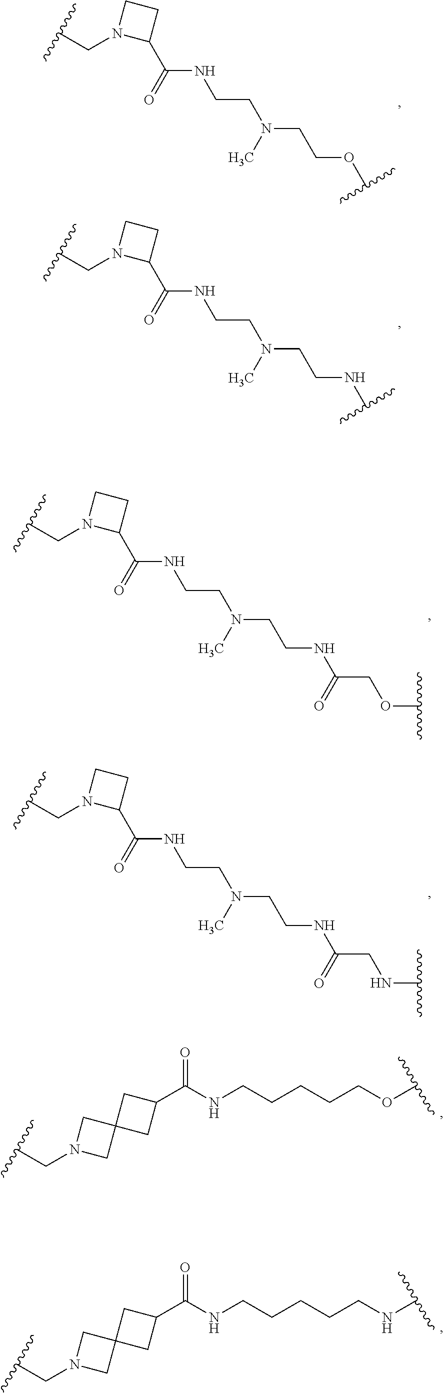 [SMILES: *CN1CCC1C(=O)NCCN(C)CCO* ; *CN1CCC1C(=O)NCCN(C)CCN* ; *CN1CCC1C(=O)NCCN(C)CCNC(=O)CO* ; *CN1CCC1C(=O)NCCN(C)CCNC(=O)CN* ; *CN1CC2(CC(C(=O)NCCCCCO*)C2)C1 ; *CN1CC2(CC(C(=O)NCCCCCN*)C2)C1]

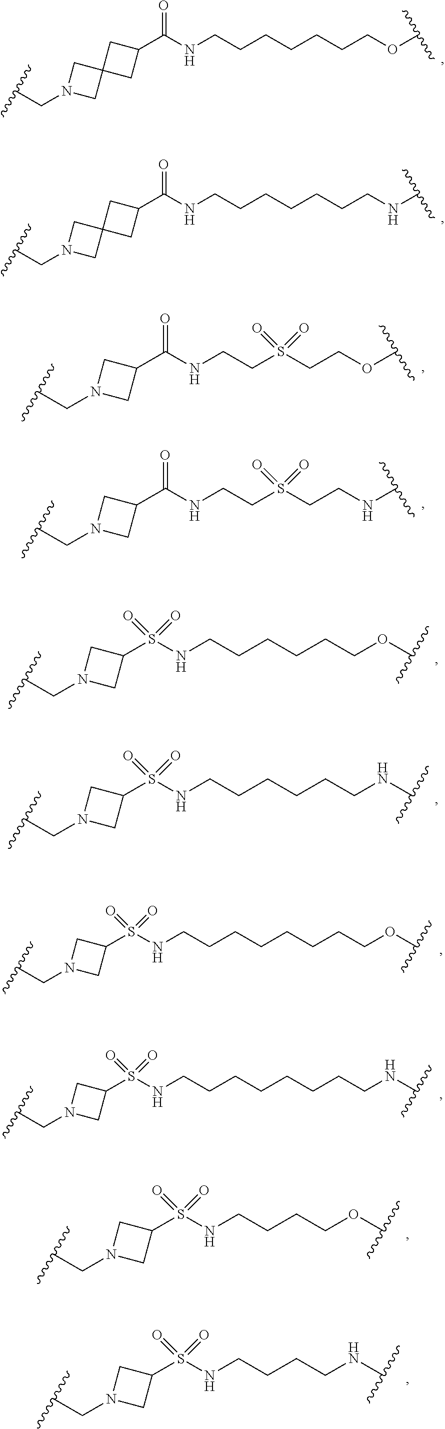

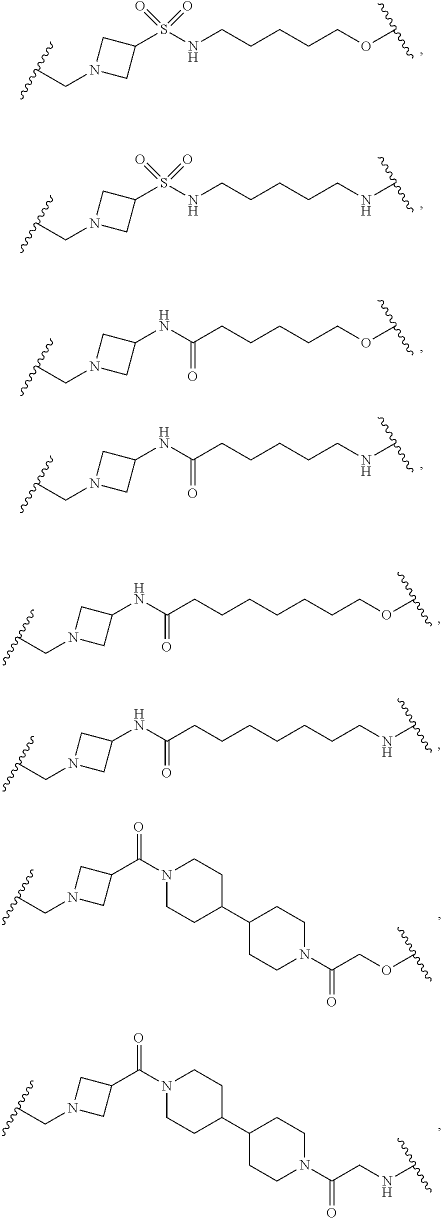

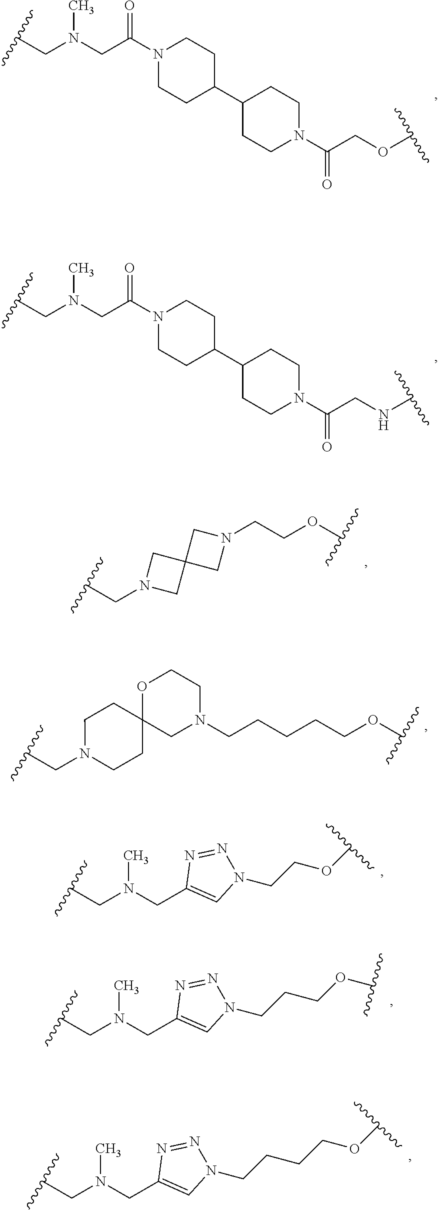

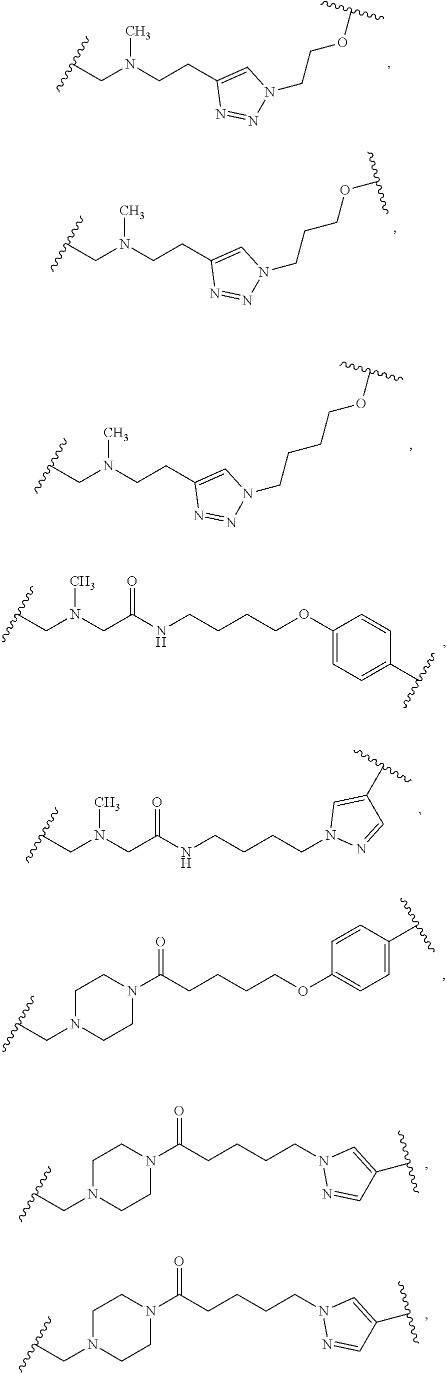

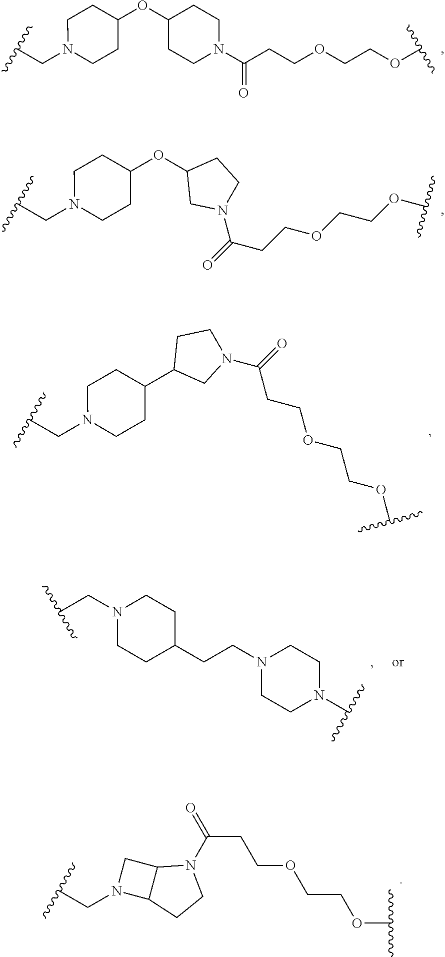 [SMILES: *CN1CCC(OC2CCN(C(=O)CCOCCO*)CC2)CC1 ; *CN1CCC(OC2CCN(C(=O)CCOCCO*)C2)CC1 ; *CN1CCC(C2CCN(C(=O)CCOCCO*)C2)CC1 ; *CN1CCC(CCN2CCN(*)CC2)CC1 ; *CN1CC2C1CCN2C(=O)CCOCCO*]

In some embodiments, the linker has the structure of

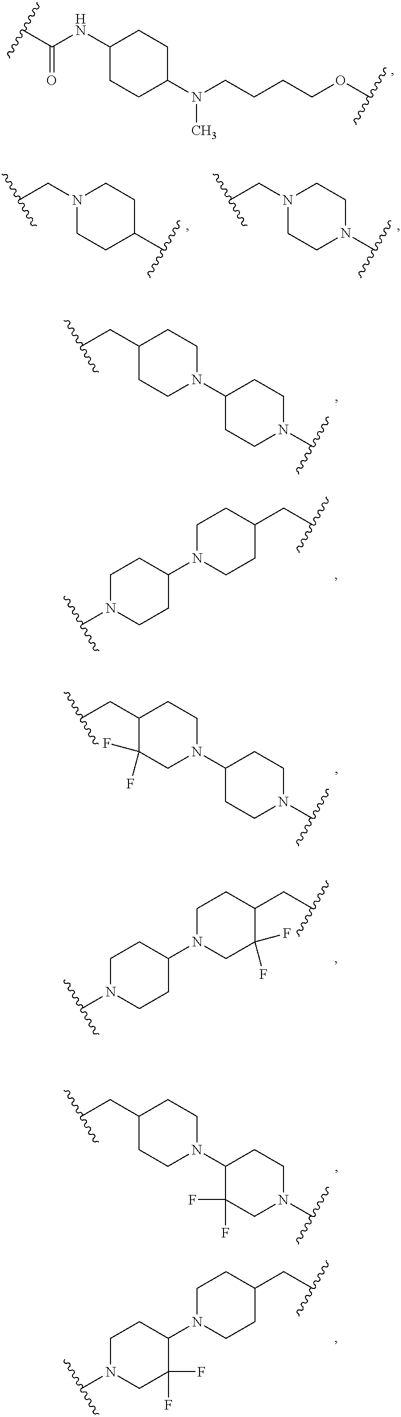

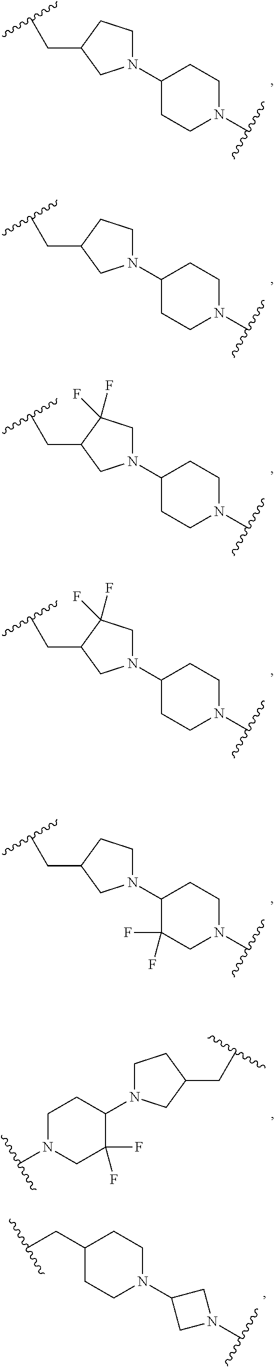

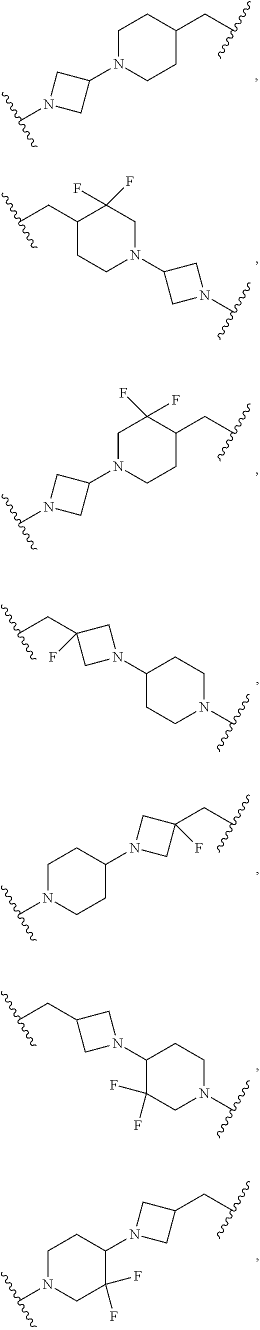

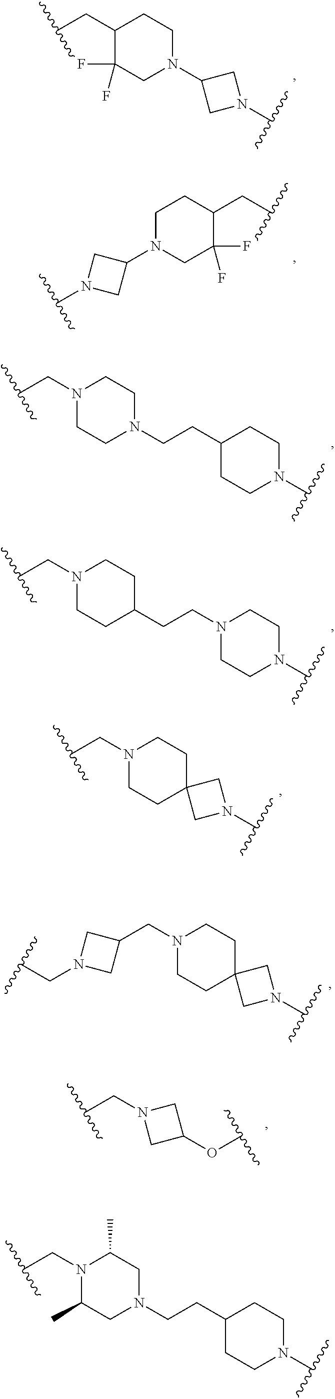

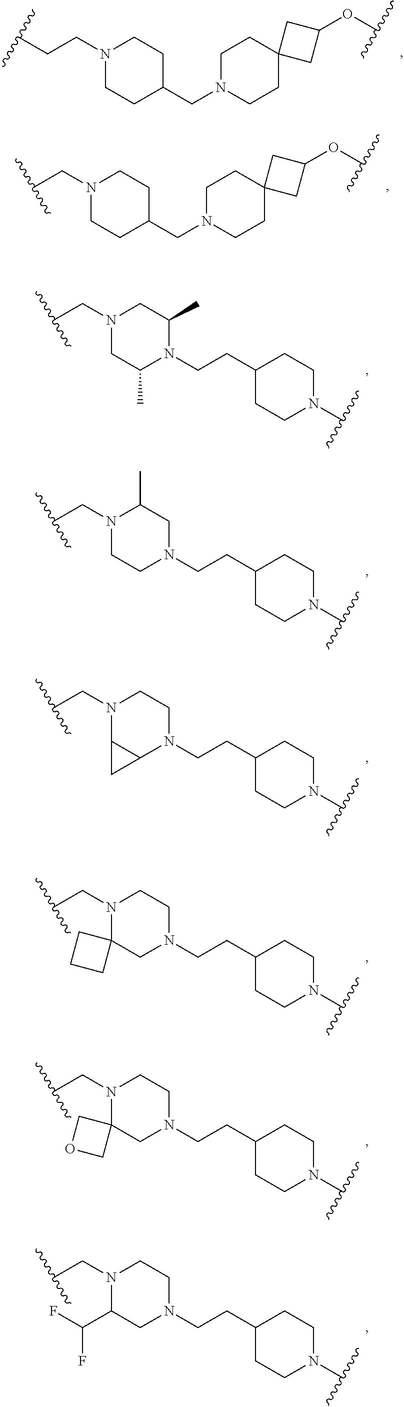

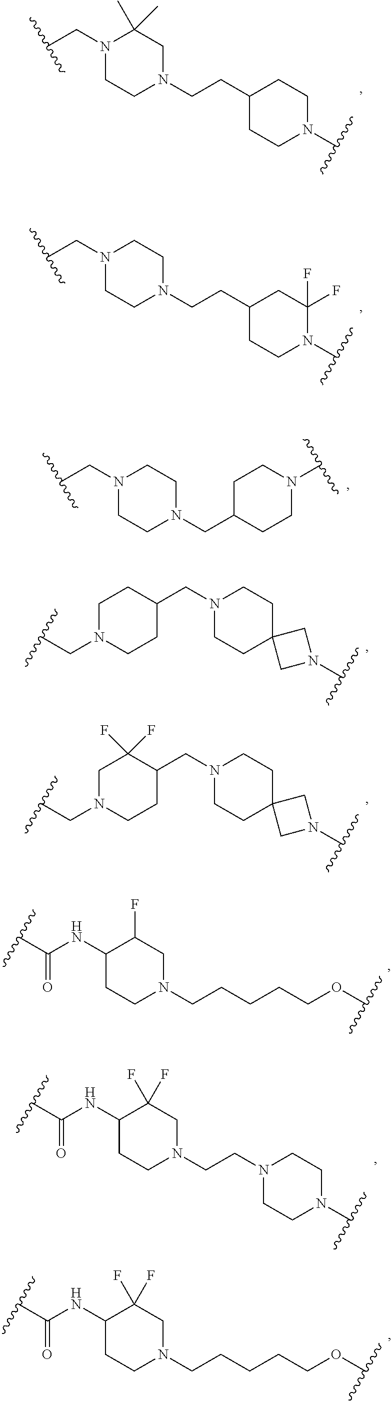

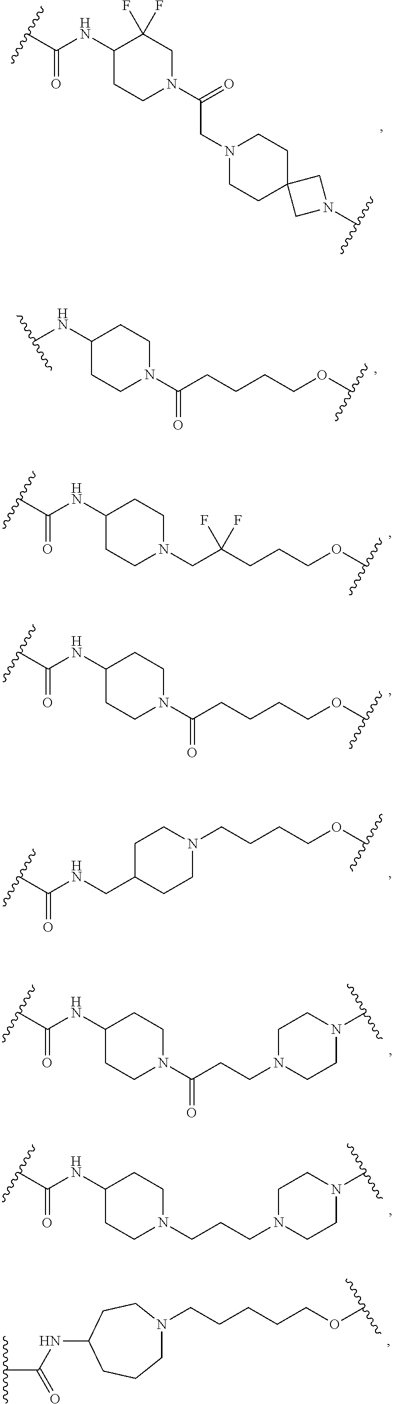

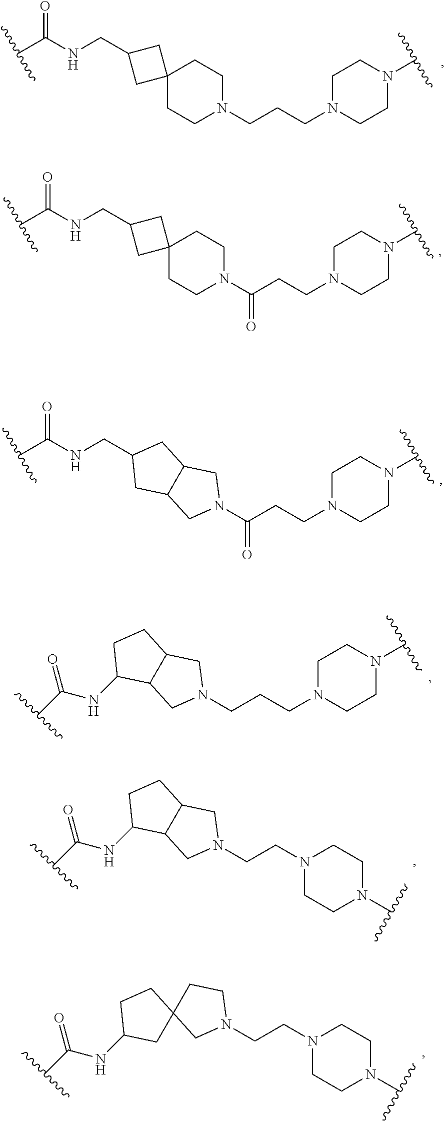 [SMILES: *C(=O)NCC1CC2(CCN(CCCN3CCN(*)CC3)CC2)C1 ; *C(=O)NCC1CC2(CCN(C(=O)CCN3CCN(*)CC3)CC2)C1 ; *C(=O)NCC1CC2CN(C(=O)CCN3CCN(*)CC3)CC2C1 ; *C(=O)NC1CCC2CN(CCCN3CCN(*)CC3)CC21 ; *C(=O)NC1CCC2CN(CCN3CCN(*)CC3)CC21 ; *C(=O)NC1CCC2(CCN(CCN3CCN(*)CC3)C2)C1]

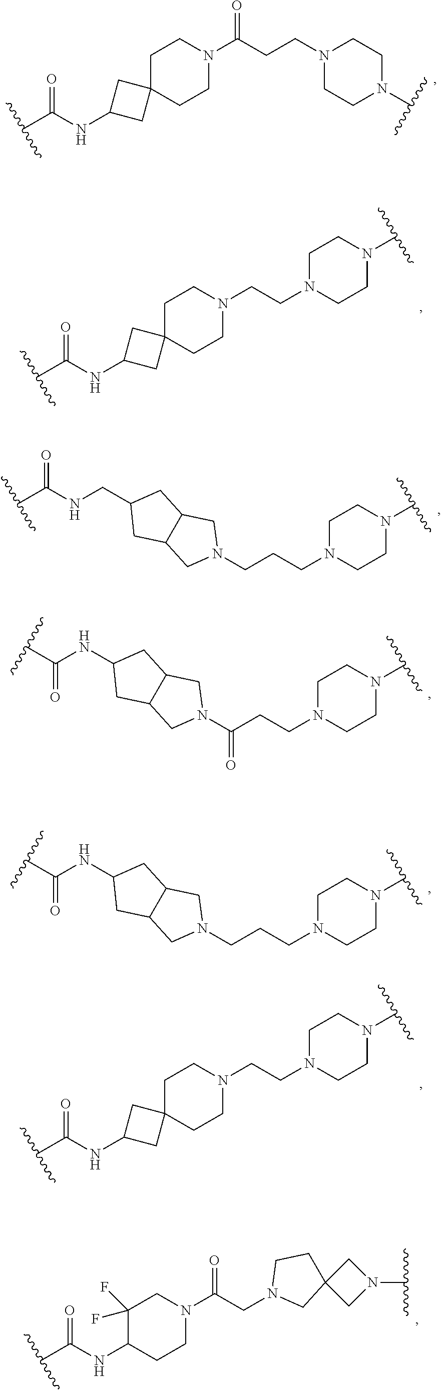

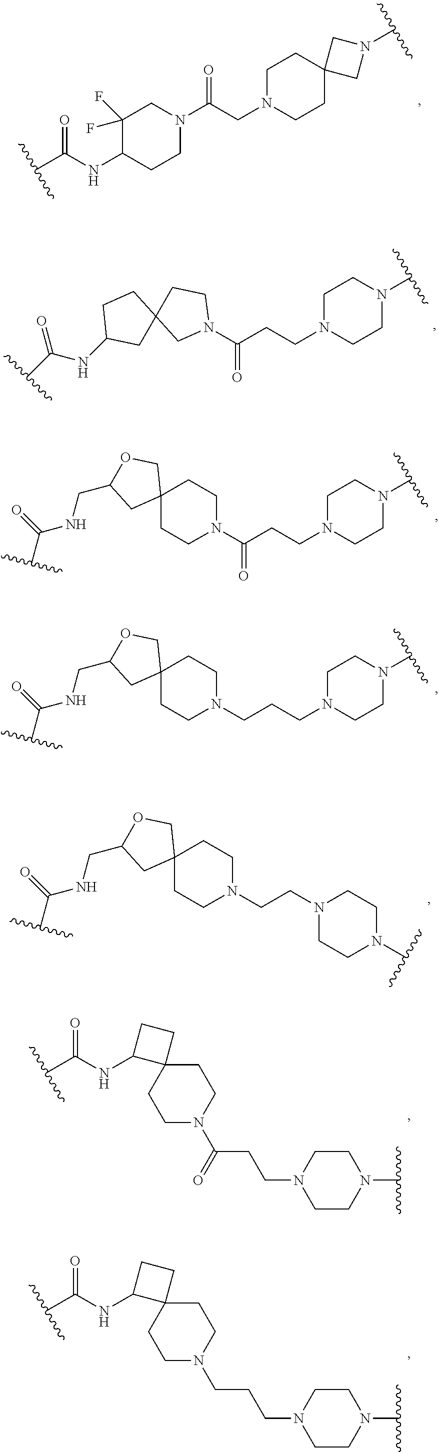

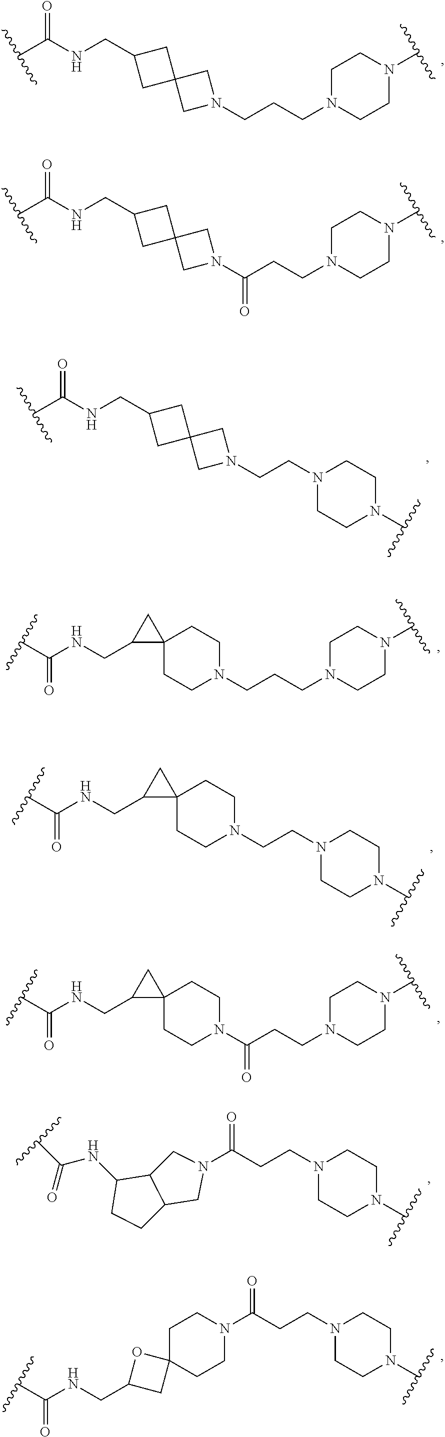

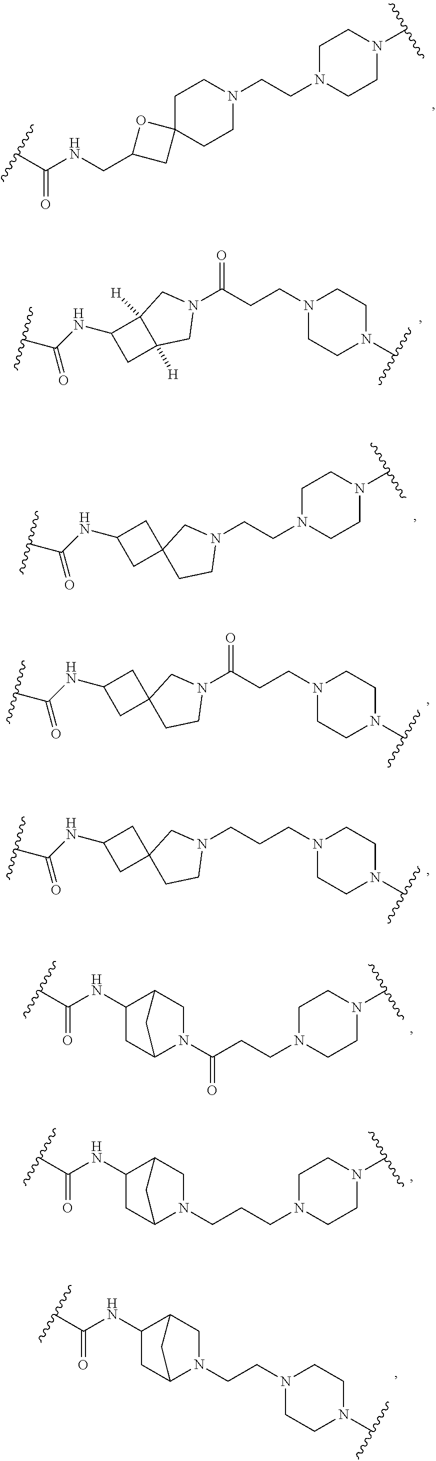

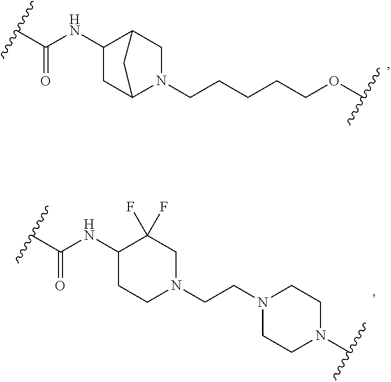 [SMILES: *OCCCCCN1CC2CC1CC2NC(*)=O ; *C(=O)NC1CCN(CCN2CCN(*)CC2)CC1(F)F]

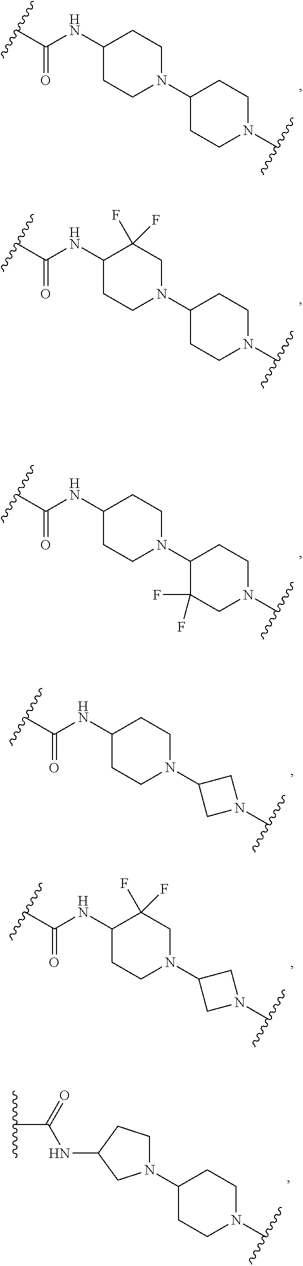 [SMILES: *C(=O)NC1CCN(C2CCN(*)CC2)CC1 ; *C(=O)NC1CCN(C2CCN(*)CC2)CC1(F)F ; *C(=O)NC1CCN(C2CCN(*)CC2(F)F)CC1 ; *C(=O)NC1CCN(C2CN(*)C2)CC1 ; *C(=O)NC1CCN(C2CN(*)C2)CC1(F)F ; *C(=O)NC1CCN(C2CCN(*)CC2)C1]

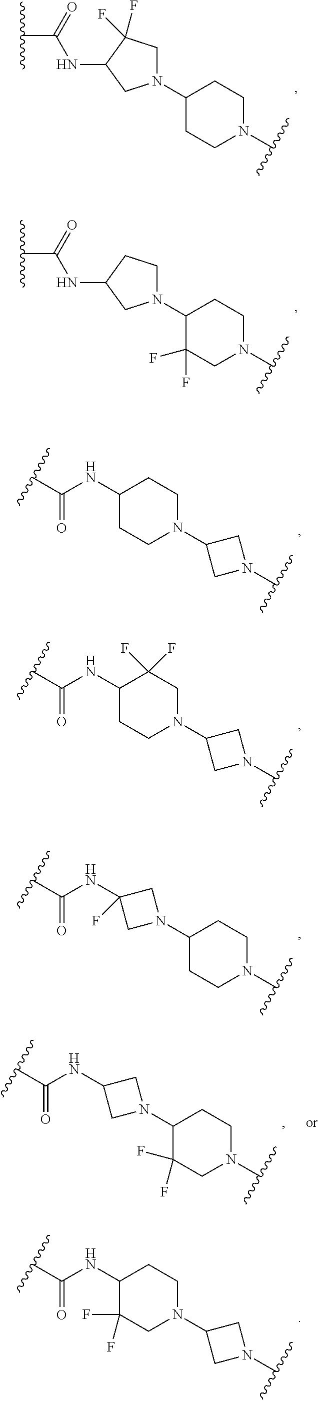

In some embodiments, the linker has the structure of:

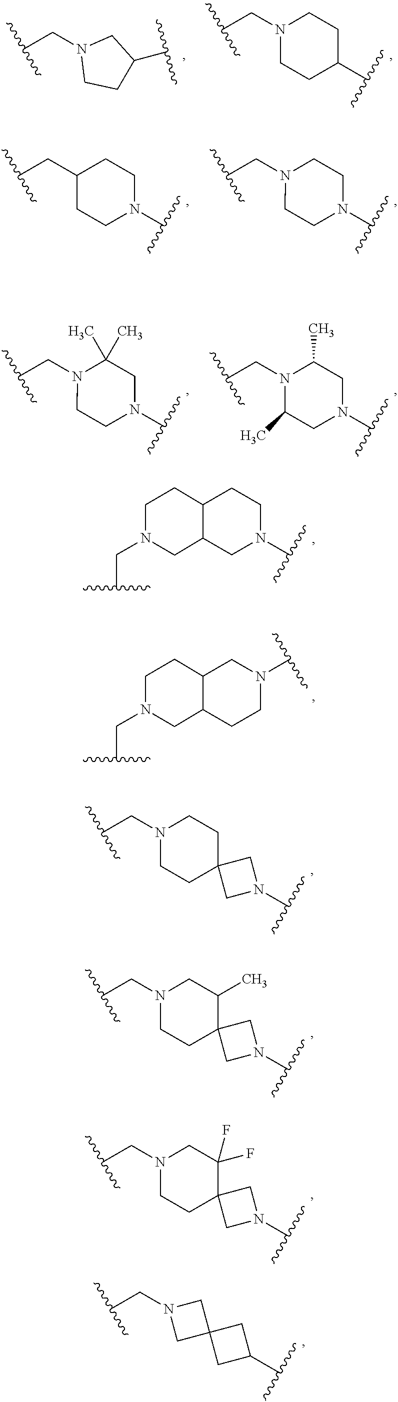

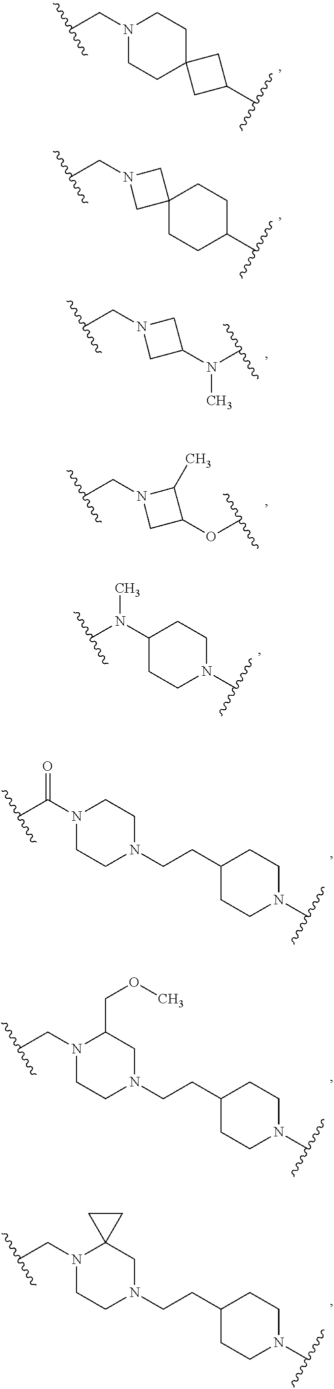

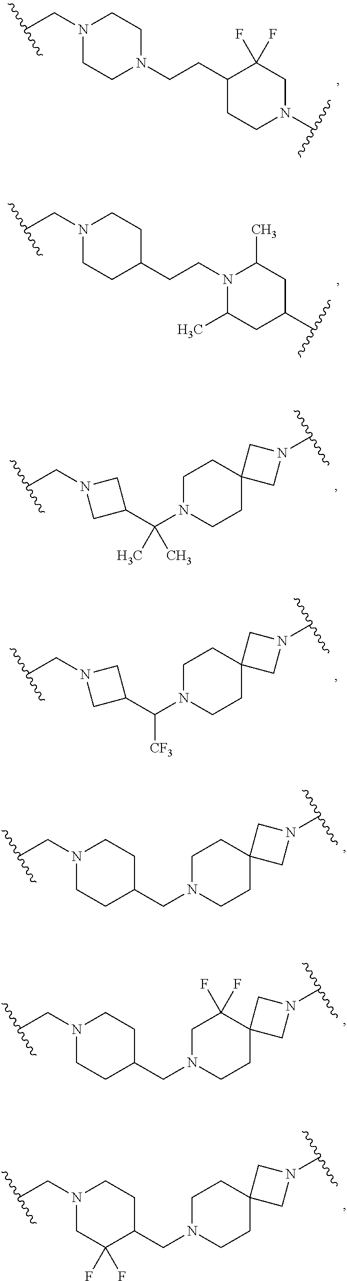

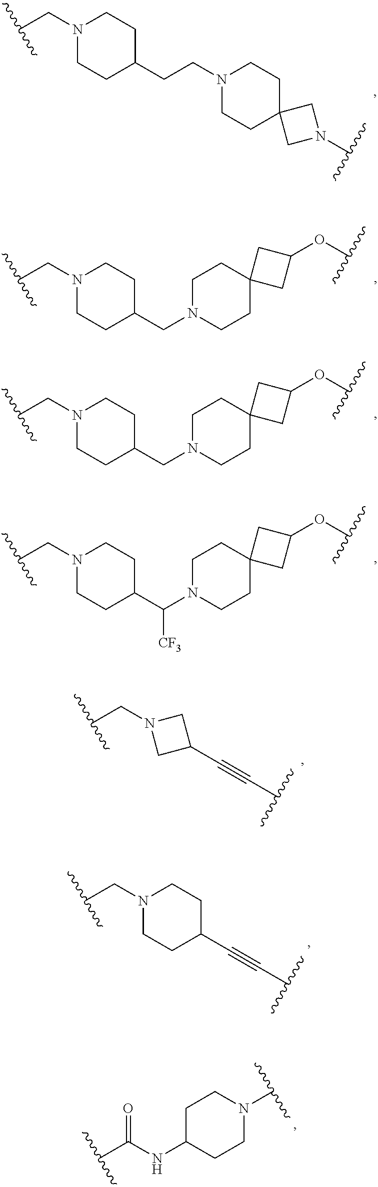

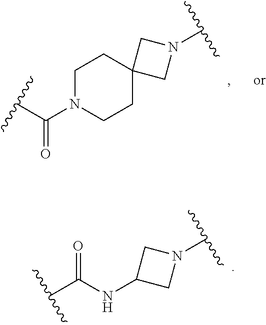 [SMILES: *C(=O)N1CCC2(CC1)CN(*)C2 ; *C(=O)NC1CN(*)C1]

In some embodiments, the linker is absent.

In some embodiments, the linker is optionally substituted C₃-C₁₀ carbocyclylene, optionally substituted C₂₋₁₀ heterocyclylene, optionally substituted C₆-C₁₀ arylene, or optionally substituted C₂-C₉ heteroarylene.

In some embodiments, the linker is optionally substituted C₃-C₁₀ carbocyclylene or optionally substituted C₂₋₁₀ heterocyclylene. In some embodiments, the linker is optionally substituted C₆-C₁₀ arylene or optionally substituted C₂-C₉ heteroarylene.

In some embodiments, the linker is optionally substituted C₂₋₁₀ heterocyclylene.

In some embodiments, the C₂-C₉ heterocyclylene is monocyclic. In some embodiments, the C₂-C₉ heterocyclylene is polycyclic.

In some embodiments, the C₂-C₉ heterocyclylene is bicyclic.

In some embodiments, the C₂-C₉ heterocyclylene is bridged. In some embodiments, the C₂-C₉ heterocyclylene is fused. In some embodiments, the C₂-C₉ heterocyclylene is spirocyclic.

In some embodiments, the linker has the structure of

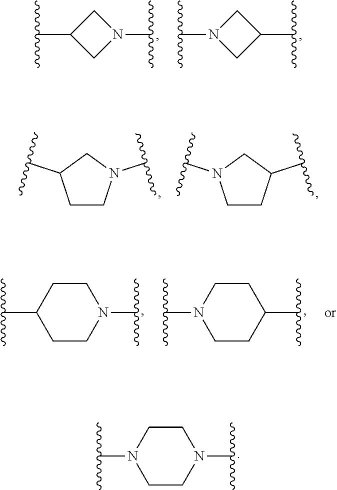

In some embodiments, the linker has the structure of

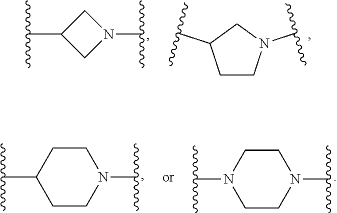

In some embodiments, the degradation moiety is a ubiquitin ligase binding moiety.

In some embodiments, the ubiquitin ligase binding moiety comprises Cereblon ligands, IAP (Inhibitors of Apoptosis) ligands, mouse double minute 2 homolog (MDM2), or von Hippel-Lindau (VHL) ligands, or derivatives or analogs thereof.

In some embodiments, the degradation moiety is a ubiquitin ligase binding moiety.

In some embodiments, the ubiquitin ligase binding moiety comprises Cereblon ligands, IAP (Inhibitors of Apoptosis) ligands, mouse double minute 2 homolog (MDM2), or von Hippel-Lindau (VHL) ligands, or derivatives or analogs thereof.

In some embodiments, the degradation moiety includes the structure of Formula Y:

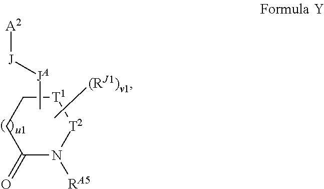

where
A² is a bond between the degradation moiety and the linker;
v1 is 0, 1, 2, 3, 4, or 5;
u1 is 1, 2, or 3;
T¹ is a bond or

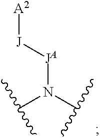

T² is

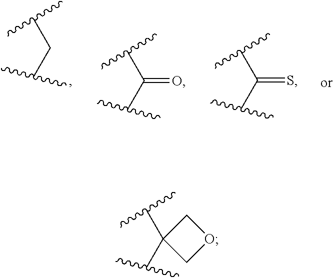

R^{5A} is H, optionally substituted C₁-C₆ alkyl, or optionally substituted C₁-C₆ heteroalkyl;
each R^{J1} is, independently, halogen, optionally substituted C₁-C₆ alkyl, or optionally substituted C₁-C₆ heteroalkyl;
J^{A} is absent, O, optionally substituted amino, optionally substituted C₁-C₆ alkyl, or optionally substituted C₁-C₆ heteroalkyl; and
J is absent, optionally substituted C₃-C₁₀ carbocyclylene, optionally substituted C₆-C₁₀ arylene, optionally substituted C₂-C₉ heterocyclylene, or optionally substituted C₂-C₉ heteroarylene, or a pharmaceutically acceptable salt thereof.

In some embodiments, T² is

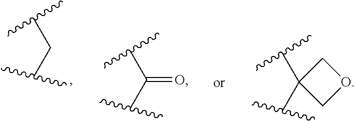

In some embodiments, T² is

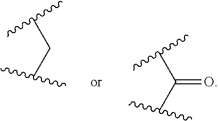

In some embodiments, T² is

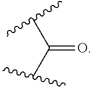 [SMILES: *C(*)=O]

In some embodiments, T² is

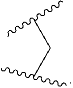 [SMILES: *C*]

In some embodiments, the structure of Formula Y has the structure of Formula Y1:

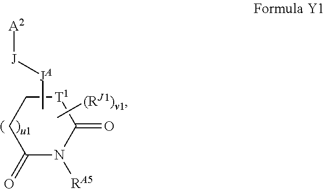

or a pharmaceutically acceptable salt thereof.

In some embodiments, T¹ is a bond. In some embodiments, T¹ is

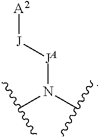

In some embodiments, the structure of Formula Y has the structure of Formula Y2:

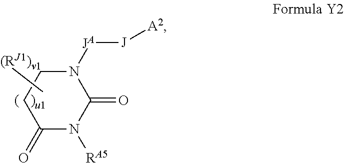

or a pharmaceutically acceptable salt thereof.

In some embodiments, the structure of Formula Y has the structure of Formula Z:

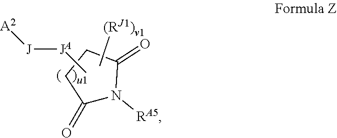

or a pharmaceutically acceptable salt thereof.

In some embodiments, u1 is 1. In some embodiments, u1 is 2. In some embodiments u1 is 3.

In some embodiments, the structure of Formula Z has the structure of Formula AA0:

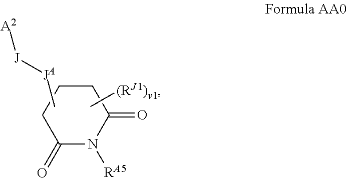

or a pharmaceutically acceptable salt thereof.

In some embodiments, the structure of Formula Z has the structure of Formula AB:

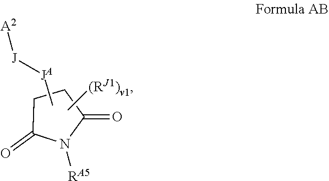

or a pharmaceutically acceptable salt thereof.

In some embodiments, the structure of Formula Z has the structure of Formula AC:

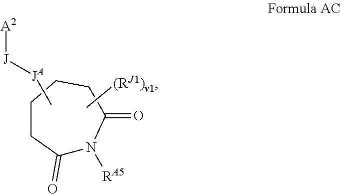

or a pharmaceutically acceptable salt thereof.

In some embodiments, J^{A} is absent. In some embodiments, J^{A} is optionally substituted C₁-C₆ alkyl. In some embodiments, J^{A} is optionally substituted C₁-C₆ heteroalkyl. In some embodiments, J^{A} is O or optionally substituted amino.

In some embodiments, J^{A} is

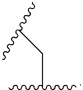 [SMILES: *C*]

In some embodiments, the structure of Formula AA0 has the structure of Formula AA0:

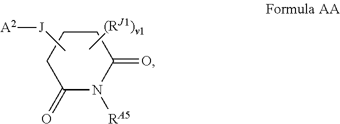

or a pharmaceutically acceptable salt thereof.

In some embodiments, v1 is 0, 1, 2, or 3. In some embodiments, v1 is 0. In some embodiments, v1 is 1. In some embodiments, v1 is 2. In some embodiments, v1 is 3.

In some embodiments, the structure of Formula AA has the structure of Formula AA1:

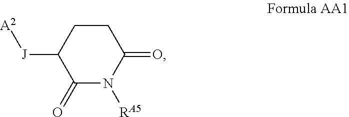

or a pharmaceutically acceptable salt thereof.

In some embodiments, the structure of Formula AB has the structure of Formula AB1:

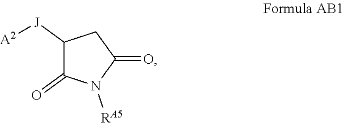

or a pharmaceutically acceptable salt thereof.

In some embodiments, the structure of Formula AC has the structure of Formula AC1:

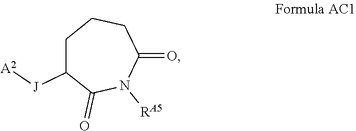

or a pharmaceutically acceptable salt thereof.

In some embodiments, J is absent. In some embodiments, J is optionally substituted C₃-C₁₀ carbocyclylene or optionally substituted C₆-C₁₀ arylene. In some embodiments, J is optionally substituted C₂-C₉ heterocyclylene or optionally substituted C₂-C₉ heteroarylene.

In some embodiments, J is optionally substituted heterocyclylene. In some embodiments, J is optionally substituted C₆-C₁₀ arylene.

In some embodiments, J is

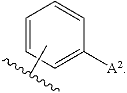

In some embodiments, the structure of Formula AA has the structure of Formula AA2:

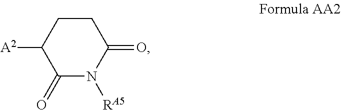

or a pharmaceutically acceptable salt thereof.

In some embodiments, the structure of Formula AA has the structure of Formula AA3:

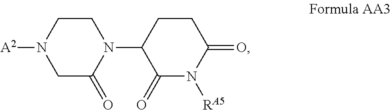

or a pharmaceutically acceptable salt thereof.

In some embodiments, the structure of Formula AA has the structure of Formula AA4:

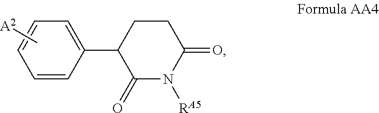

or a pharmaceutically acceptable salt thereof.

In some embodiments, R^{A5} is H or optionally substituted C₁-C₆ alkyl. In some embodiments, R^{A5} is optionally substituted C₁-C₆ heteroalkyl.

In some embodiments, R^{A5} is H or methyl. In some embodiments, R^{A5} is H. In some embodiments, R^{A5} is methyl. In some embodiments, R^{A5} is

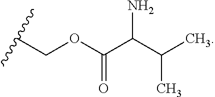 [SMILES: *COC(=O)C(N)C(C)C]

In some embodiments, the structure of Formula AA has the structure of Formula A:

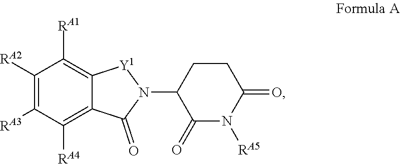

where
Y¹ is

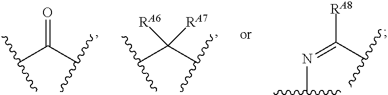

R^{A5} is H, optionally substituted C₁-C₆ alkyl, or optionally substituted C₁-C₆ heteroalkyl;
R^{A6} is H or optionally substituted C₁-C₆ alkyl; and R^{A7} is H or optionally substituted C₁-C₆ alkyl; or R^{A6} and R^{A7}, together with the carbon atom to which each is bound, combine to form optionally substituted C₃-C₆ carbocyclyl or optionally substituted C₂-C₅ heterocyclyl; or R^{A6} and R^{A7}, together with the carbon atom to which each is bound, combine to form optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
each of R^{A1}, R^{A2}, R^{A3} and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

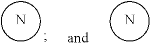

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A², where one of R^{A1}, R^{A2}, R^{A3}, and R^{A4} is A², or

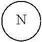

is substituted with A², or a pharmaceutically acceptable salt thereof.

In some embodiments, each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

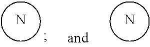

and is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A², where one of R^{A1}, R^{A2}, R^{A3} and R^{A4} is A², or

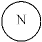

is substituted with A², or a pharmaceutically acceptable salt thereof.

In some embodiments, each of R^{A1}, R^{A2}, R^{A3}, and R^{A4} is, H, A², halogen, optionally substituted C₁-C₆ alkyl, optionally substituted C₁-C₆ heteroalkyl, optionally substituted —O—C₃-C₆ carbocyclyl, hydroxyl, optionally substituted amino; or R^{A1} and R^{A2}, R^{A2} and R^{A3}, or R^{A3} and R^{A4}, together with the carbon atoms to which each is attached, combine to form

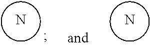

and is optionally substituted C₂-C₉ heterocyclyl, which is optionally substituted with A², where one of R^{A1}, R^{A2}, R^{A3}, and R^{A4} is A², or

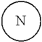

is substituted with A².

In some embodiments, each of R^{A1}, R^{A2}, R^{A3} and R^{A4} is, independently, H, A², F,

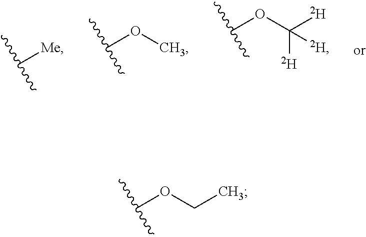

or R^{A1} and R^{A2}, R^{A2} and R^{A3}, or R^{A3} and R^{A4}, together with the carbon atoms to which each is attached, combine to form

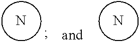

is optionally substituted C₂-C₉ heterocyclyl, which is optionally substituted with A², where one of R^{A1}, R^{A2}, R^{A3}, and R^{A4} is A², or

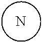

is substituted with A².

In some embodiments, R^{A1} is A². In some embodiments, R^{A2} is A². In some embodiments, R^{A3} is A². In some embodiments, R^{A4} is A². In some embodiments, R^{A5} is A².

In some embodiments, R^{A5} is H or optionally substituted C₁-C₆ alkyl.

In some embodiments, R^{A5} is H or

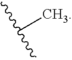

In some embodiments, R^{A5} is H. In some embodiments, R^{A5} is

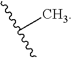

In some embodiments, Y¹ is

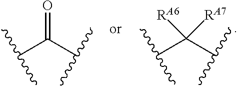

In some embodiments, Y¹ is

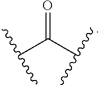 [SMILES: *C(*)=O]

In some embodiments, Y¹ is

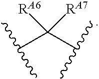

In some embodiments, each of R^{A6} and R^{A7} is, independently, H, F,

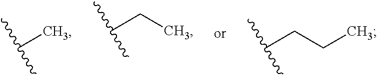

or R^{A6} and R^{A7}, together with the carbon atom to which each is bound, combine to form

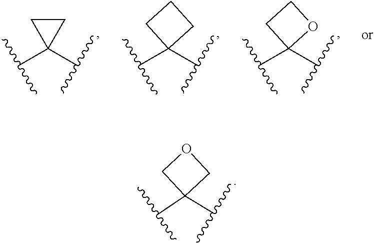 [SMILES: *C1(*)CC1 ; *C1(*)CCC1 ; *C1(*)CCO1 ; *C1(*)COC1]

In some embodiments, R^{A6} is H and R^{A7} is H.

In some embodiments, Y¹ is

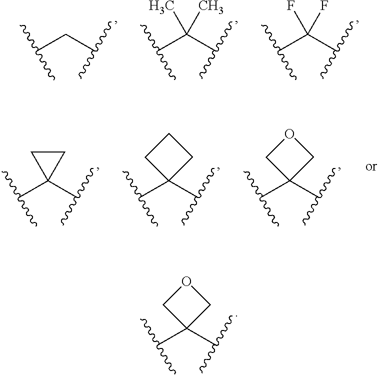

In some embodiments, Y¹ is

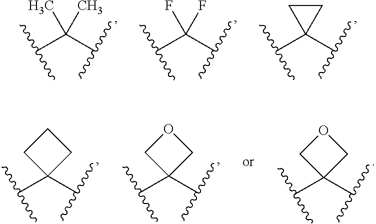

In some embodiments, Y¹ is

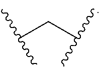 [SMILES: *C*]

In some embodiments, the structure of Formula A has the structure of Formula A1:

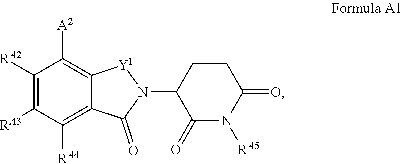

or a pharmaceutically acceptable salt thereof.

In some embodiments, the structure of Formula A has the structure of Formula A2:

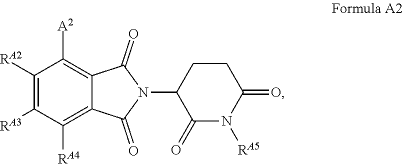

or a pharmaceutically acceptable salt thereof.

In some embodiments, the structure of Formula A has the structure of Formula A3:

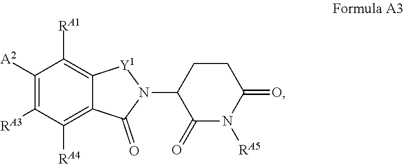

or a pharmaceutically acceptable salt thereof.

In some embodiments, the structure of Formula A has the structure of Formula A4:

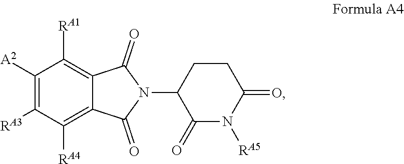

or a pharmaceutically acceptable salt thereof.

In some embodiments, the structure of Formula A has the structure of Formula A5:

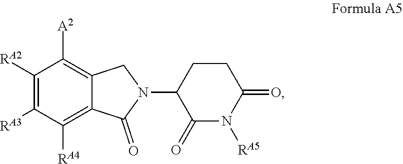

or a pharmaceutically acceptable salt thereof.

In some embodiments, the structure of Formula A has the structure of Formula A6:

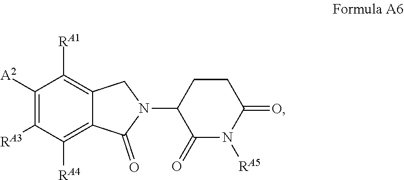

or a pharmaceutically acceptable salt thereof.

In some embodiments, the structure of Formula A has the structure of Formula A7:

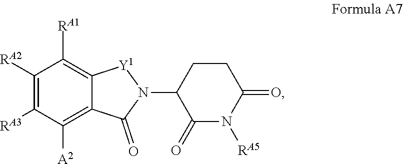

or a pharmaceutically acceptable salt thereof.

In some embodiments, the structure of Formula A has the structure of Formula A8:

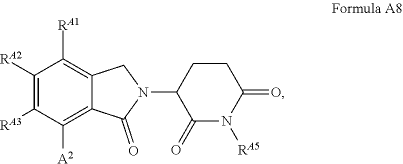

or a pharmaceutically acceptable salt thereof.

In some embodiments, the structure of Formula A has the structure of Formula A9:

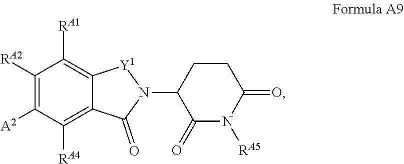

or a pharmaceutically acceptable salt thereof.

In some embodiments, the structure of Formula A has the structure of Formula A10:

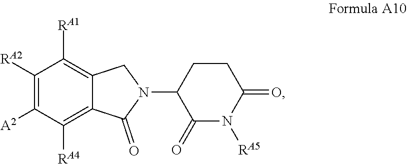

or a pharmaceutically acceptable salt thereof.

In some embodiments, wherein the structure of Formula A is

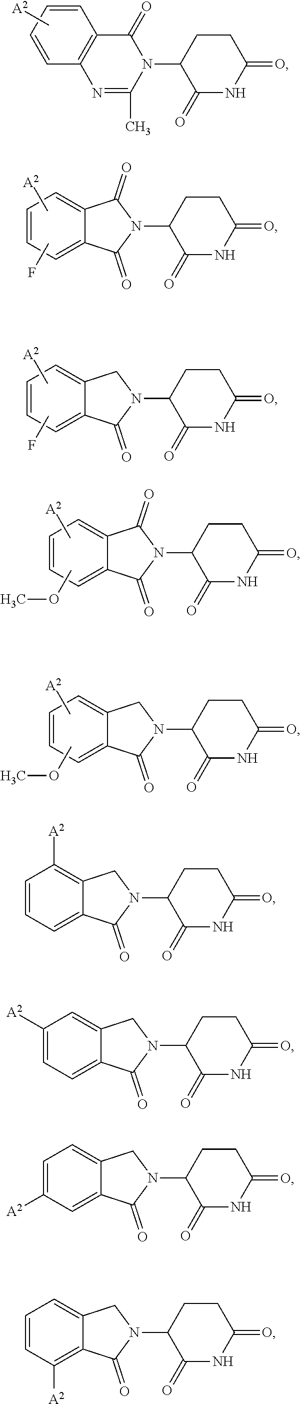

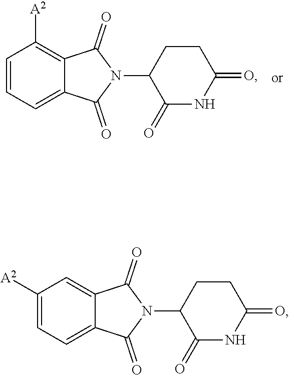

or derivative or analog thereof.

In some embodiments, the structure of Formula A is

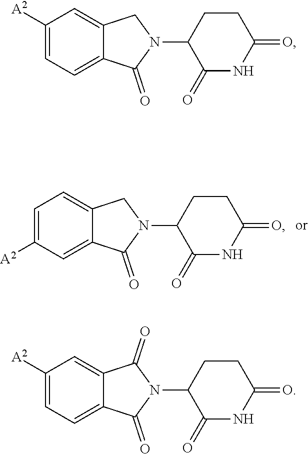

In some embodiments, the structure of Formula A is

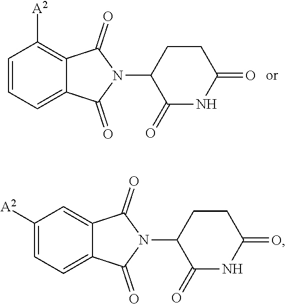

or derivative or analog thereof.

In some embodiments,

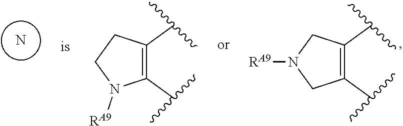

where R^{A9} is H, A², optionally substituted C₁-C₆ alkyl, or optionally substituted C₁-C₆ heteroalkyl.

In some embodiments, the structure of Formula A is

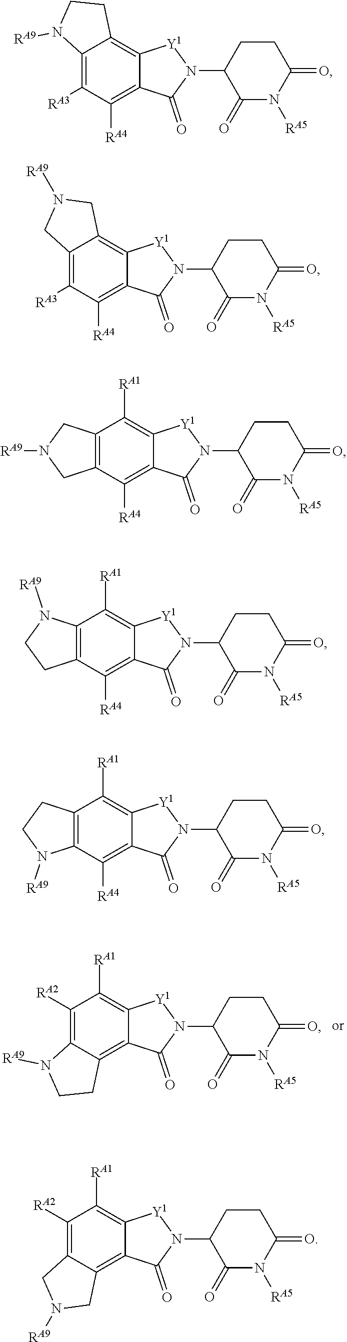

In some embodiments, R^{A9} is H, A², or optionally substituted C₁-C₆ alkyl. In some embodiments, R^{A9} is H, A², or methyl. In some embodiments, R^{9A} is H. In some embodiments, R^{9A} is methyl. In some embodiments, R^{A9} is A².

In some embodiments, the structure of Formula A is

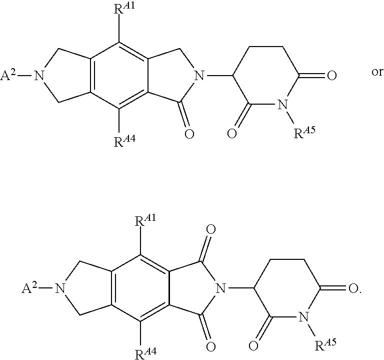

In some embodiments, the structure of Formula AA has the structure of Formula B:

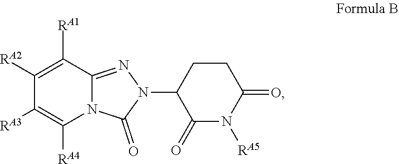

where
R^{A5} is H, optionally substituted C₁-C₆ alkyl, or optionally substituted C₁-C₆ heteroalkyl;
each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

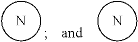

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A², where one of R^{A1}, R^{A2} R^{A3}, and R^{A4} is A², or

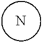

is substituted with A², or a pharmaceutically acceptable salt thereof.

In some embodiments, each of R^{A1}, R^{A2}, R^{A3}, and R^{A4} is, H, A², halogen, optionally substituted C₁-C₆ alkyl, optionally substituted C₁-C₆ heteroalkyl, optionally substituted —O—C₃-C₆ carbocyclyl, hydroxyl, optionally substituted amino; or R^{A1} and R^{A2}, R^{A2} and R^{A3}, or R^{A3} and R^{A4}, together with the carbon atoms to which each is attached, combine to form

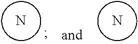

is optionally substituted C₂-C₉ heterocyclyl, which is optionally substituted with A², where one of R^{A1}, R^{A2}, R^{A3} and R^{A4} is A², or

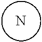

is substituted with A².

In some embodiments, each of R^{A1}, R^{A2}, R^{A3} and R^{A4} is, independently, H, A², F,

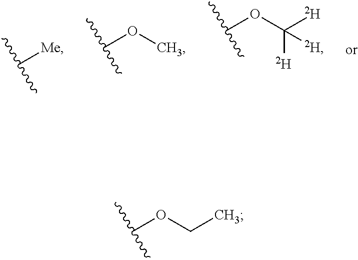

or R^{A1} and R^{A2}, R^{A2} and R^{A3}, or R^{A3} and R^{A4}, together with the carbon atoms to which each is attached, combine to form

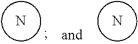

is optionally substituted C₂-C₉ heterocyclyl, which is optionally substituted with A², where one of R^{A1}, R^{A2}, R^{A3} and R^{A4} is A², or

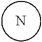

is substituted with A².

In some embodiments, R^{A1} is A². In some embodiments, R^{A2} is A². In some embodiments, R^{A3} is A². In some embodiments, R^{A4} is A². In some embodiments, R^{A5} is A².

In some embodiments, R^{A5} is H or optionally substituted C₁-C₆ alkyl.

In some embodiments, R^{A5} is H or

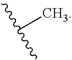

In some embodiments, R^{A5} is H. In some embodiments, R^{A5} is

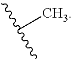

In some embodiments, the structure of Formula B has the structure of Formula B1:

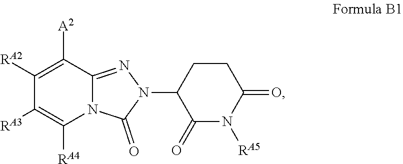

or a pharmaceutically acceptable salt thereof.

In some embodiments, the structure of Formula B has the structure of Formula B2:

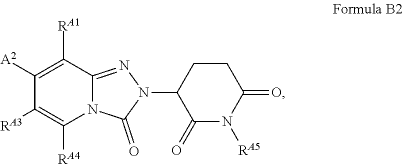

or a pharmaceutically acceptable salt thereof.

In some embodiments, the structure of Formula B has the structure of Formula B3:

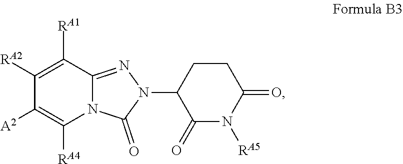

or a pharmaceutically acceptable salt thereof.

In some embodiments, the structure of Formula B has the structure of Formula B4:

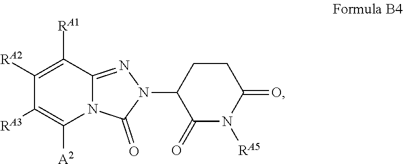

or a pharmaceutically acceptable salt thereof.

In some embodiments, the structure of Formula B is

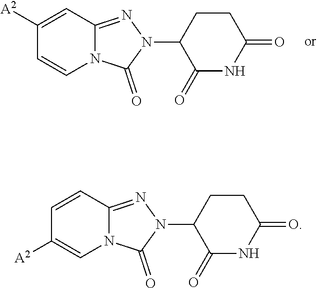

In some embodiments, the structure of Formula B is

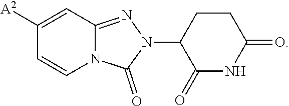

In some embodiments, the structure of Formula B is

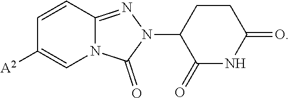

In some embodiments, the ubiquitin ligase binding moiety comprises a von Hippel-Lindau ligand.

In some embodiments, the von Hippel-Lindau ligand has the structure of

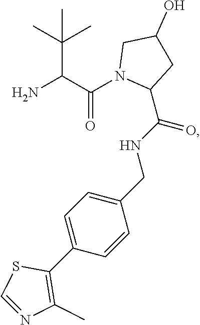 [SMILES: Cc1ncsc1-c1ccc(CNC(=O)C2CC(O)CN2C(=O)C(N)C(C)(C)C)cc1]

or derivative or analog thereof.

In some embodiments, the degradation moiety includes the structure of Formula C:

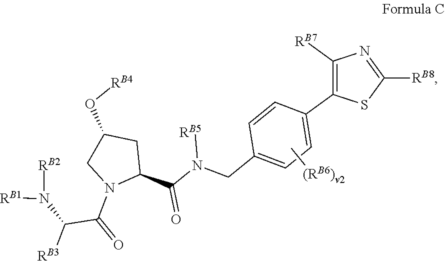

where
R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and
each of R^{B7} and R^{B8} is, independently, H, halogen, optionally substituted C₁-C₆ alkyl, or optionally substituted C₆-C₁₀ aryl,
where one of R^{B1} and R^{B3} is A², or a pharmaceutically acceptable salt thereof.

In some embodiments, the structure of Formula C is

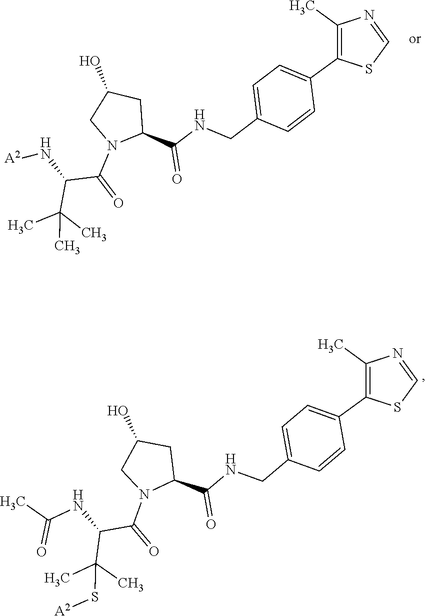

or derivative or analog thereof.

In some embodiments, the structure of Formula C is

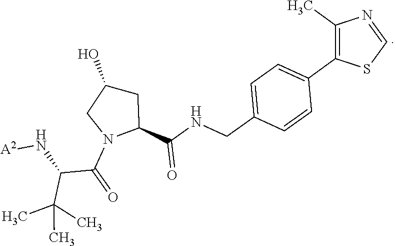

In some embodiments, the degrader moiety includes the structure of Formula D:

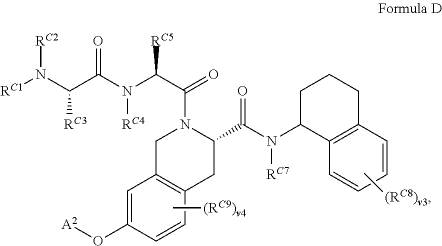

where
A² is a bond between B and the linker;
each of R^{C1}, R^{C2}, and R^{C7} is, independently, H, optionally substituted C₁-C₆ alkyl, or optionally substituted C₁-C₆ heteroalkyl;
R^{C3} is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{C5} is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
v3 is 0, 1, 2, 3, or 4;
each R^{C8} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
v4 is 0, 1, 2, 3, or 4; and
each R^{C9} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the structure of Formula D is

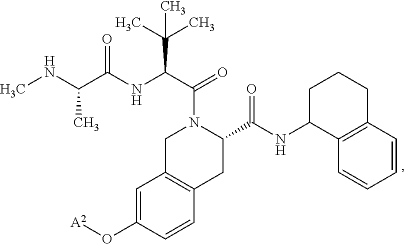

or derivative or analog thereof.

In some embodiments, the degrader moiety includes the structure of Formula E:

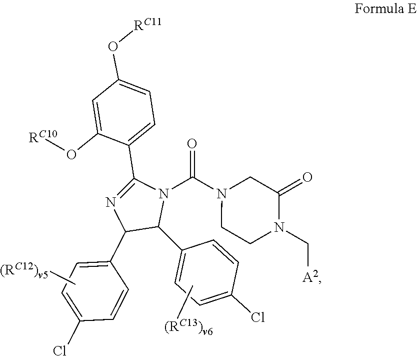

where
A² is a bond between B and the linker;
each of R^{C10} and R^{C11} is, independently, H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
v5 is 0, 1, 2, 3, or 4;
each R^{C12} is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
v6 is 0, 1, 2, 3, or 4; and
each R²¹ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the structure of Formula E is

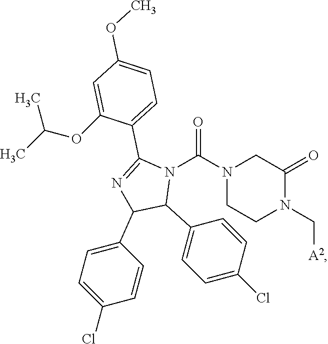

or derivative or analog thereof.

In some embodiments, the degradation moiety includes the structure of Formula FA:

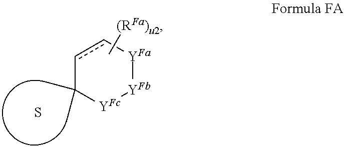

where

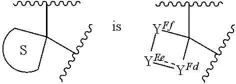

or a bicyclic moiety which is substituted with A² and substituted with one or more groups independently selected from H, R^{FF1}, and oxo;
[symbol] is a single bond or a double bond;
u2 is 0, 1, 2, or 3;
A² is a bond between the degrader and the linker;
Y^{Fa} is CR^{Fb}R^{Fc}, C═O, C═S, C═CH₂, SO₂, S(O), P(O)Oalkyl, P(O)NHalkyl, P(O)N(alkyl)₂, P(O)alkyl, P(O)OH, P(O)NH₂;
Y^{Fb} is NH, NR^{FF1}, CH₂, CH^{RFF1}, C(R^{FF1})₂, O, or S;
Y^{Fc} is CR^{Fd}R^{Fe}, C═O, C═S, C═CH₂, SO₂, S(O), P(O)Oalkyl, P(O)NHalkyl, P(O)N(alkyl)₂, P(O)alkyl, P(O)OH, P(O)NH₂;
each of R^{Fb}, R^{Fc}, R^{Fd} and R^{Fe} is, independently, H, alkyl, aliphatic, heteroaliphatic, aryl, heteroaryl, carbocyclyl, hydroxyl, alkoxy, amino, —NHalkyl, or —Nalkyl₂;
or R^{Fb} and R^{Fc}, together with the carbon atom to which each is attached, combine to form a 3-, 4-, 5-, or 6-membered spirocarbocyclylene, or a 4-, 5-, or 6-membered spiroheterocyclylene comprising 1 or 2 heteroatoms selected from N and O;
or R^{Fd} and R^{Fe}, together with the carbon atom to which each is attached, combine to form a 3-, 4-, 5-, or 6-membered spirocarbocyclylene, or a 4-, 5-, or 6-membered spiroheterocyclylene comprising 1 or 2 heteroatoms selected from N and O; and
or R^{Fd} and R^{Fb}, together with the carbon atoms to which each is attached, combine to form a 1, 2, 3, or 4 carbon bridged ring;
each of Y^{Fd} and Y^{Ff} is, independently, CH, CHR^{FF2}, C(R^{FF2})₂, C(O), N, NH, NR^{FF3}, O, S, or S(O)
Y^{Fe} is a bond or a divalent moiety attached to Y^{Fd} and Y^{Ff} that contains 1 to 5 contiguous carbon atoms that form a 3 to 8-membered ring,
   wherein 1, 2, or 3 carbon atoms can be replaced with a nitrogen, oxygen, or sulfur atom;
   wherein one of the ring atoms is substituted with A² and the others are substituted with one or more groups independently selected from H and R^{FF1} and
   wherein the contiguous atoms of Y^{Fe} can be attached through a single or double bond;
each R^{FF1} is, independently, H, alkyl, alkenyl, alkynyl, aliphatic, heteroaliphatic, carbocyclyl, halogen, hydroxyl, amino, cyano, alkoxy, aryl, heteroaryl, heterocyclyl, alkylamino, alkylhydroxyl, or haloalkyl;
each R^{FF2} is, independently, alkyl, alkene, alkyne, halogen, hydroxyl, alkoxy, azide, amino, —C(O)H, —C(O)OH, —C(O)(aliphatic, including alkyl), —C(O)O(aliphatic, including alkyl), —NH(aliphatic, including alkyl), —N(aliphatic including alkyl)(aliphatic including alkyl), —NHSO₂alkyl, —N(alkyl)SO₂alkyl, —NHSO₂aryl, —N(alkyl)SO₂aryl, —NHSO₂alkenyl, —N(alkyl)SO₂alkenyl, —NHSO₂alkynyl, —N(alkyl)SO₂alkynyl, aliphatic, heteroaliphatic, aryl, heteroaryl, heterocyclic, carbocyclic, cyano, nitro, nitroso, —SH, —Salkyl, or haloalkyl; and
R^{FF3} is alkyl, alkenyl, alkynyl, —C(O)H, —C(O)OH, —C(O)alkyl, or —C(O)Oalkyl,
wherein if Y^{Fd} or Y^{Ff} is substituted with A², then Y^{Fe} is a bond, or a pharmaceutically acceptable salt thereof.

In some embodiments, the compound of Formula FA has the structure of Formula FA1:

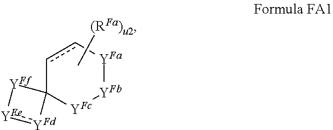

or a pharmaceutically acceptable salt thereof.

In some embodiments, the degradation moiety includes the structure of Formula FB:

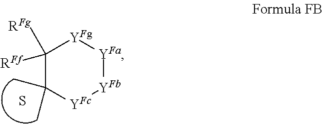

where

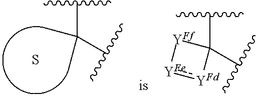

or a bicyclic moiety which is substituted with A² and substituted with one or more groups independently selected from H, R^{FF1}, and oxo;
A² is a bond between the degrader and the linker;
Y^{Fa} is CR^{Fb}R^{Fc}, C═O, C═S, C═CH₂, SO₂, S(O), P(O)Oalkyl, P(O)NHalkyl, P(O)N(alkyl)₂, P(O)alkyl, P(O)OH, P(O)NH₂;
each of Y^{Fb} and Y^{Fg} is, independently, NH, NR^{FF1}, CH₂, CHR^{FF1}, C(R^{FF1})₂, O, or S;
Y^{Fc} is CR^{Fd}R^{Fe}, C═O, C═S, C═CH₂, SO₂, S(O), P(O)Oalkyl, P(O)NHalkyl, P(O)N(alkyl)₂, P(O)alkyl, P(O)OH, P(O)NH₂;
each of R^{Fb}, R^{Fc}, R^{Fd}, R^{Fe}, R^{Ff} and R^{Fg} is, independently, H, alkyl, aliphatic, heteroaliphatic, aryl, heteroaryl, carbocyclyl, hydroxyl, alkoxy, amino, —NHalkyl, or —Nalkyl₂;
or R^{Fb} and R^{Fc}, together with the carbon atom to which each is attached, combine to form a 3-, 4-, 5-, or 6-membered spirocarbocyclylene, or a 4-, 5-, or 6-membered spiroheterocyclylene comprising 1 or 2 heteroatoms selected from N and O;
or R^{Fd} and R^{Fe}, together with the carbon atom to which each is attached, combine to form a 3-, 4-, 5-, or 6-membered spirocarbocyclylene, or a 4-, 5-, or 6-membered spiroheterocyclylene comprising 1 or 2 heteroatoms selected from N and O;
or R^{Ff} and R^{Fg}, together with the carbon atom to which each is attached, combine to form a 3-, 4-, 5-, or 6-membered spirocarbocyclylene, or a 4-, 5-, or 6-membered spiroheterocyclylene comprising 1 or 2 heteroatoms selected from N and O;
or R^{Fd} and R^{Fb}, together with the carbon atoms to which each is attached, combine to form a 1, 2, 3, or 4 carbon bridged ring;
or R^{Fd} and R^{Ff}, together with the carbon atoms to which each is attached, combine to form a 1, 2, 3, or 4 carbon bridged ring;
or R^{Fb} and R^{Fg}, together with the carbon atoms to which each is attached, combine to form a 1, 2, 3, or 4 carbon bridged ring;
each of Y^{Fd} and Y^{FF} is, independently, CH₂, CHR^{FF2}, C(R^{FF2})₂, C(O), N, NH, NR^{FF3}, O, S, or S(O)
Y^{Fe} is a bond or a divalent moiety attached to Y^{Fd} and Y^{Ff} that contains 1 to 5 contiguous carbon atoms that form a 3 to 8-membered ring,
   wherein 1, 2, or 3 carbon atoms can be replaced with a nitrogen, oxygen, or sulfur atom;
   wherein one of the ring atoms is substituted with A² and the others are substituted with one or more groups independently selected from H and R^{FF1}; and
   wherein the contiguous atoms of Y^{Fe} can be attached through a single or double bond;
each R^{FF1} is, independently, H, alkyl, alkenyl, alkynyl; aliphatic, heteroaliphatic, carbocyclyl, halogen, hydroxyl, amino, cyano, alkoxy, aryl, heteroaryl, heterocyclyl, alkylamino, alkylhydroxyl, or haloalkyl;
each R^{FF2} is, independently, alkyl, alkene, alkyne, halogen, hydroxyl, alkoxy, azide, amino, —C(O)H, —C(O)OH, —C(O)(aliphatic, including alkyl), —C(O)O(aliphatic, including alkyl), —NH(aliphatic, including alkyl), —N(aliphatic including alkyl)(aliphatic including alkyl), —NHSO₂alkyl, —N(alkyl)SO₂alkyl, —NHSO₂aryl, —N(alkyl)SO₂aryl, —NHSO₂alkenyl, —N(alkyl)SO₂alkenyl, —NHSO₂alkynyl, —N(alkyl)SO₂alkynyl, aliphatic, heteroaliphatic, aryl, heteroaryl, hetercyclic, carbocyclic, cyano, nitro, nitroso, —SH, —Salkyl, or haloalkyl; and
R^{FF3} is alkyl, alkenyl, alkynyl, —C(O)H, —C(O)OH, —C(O)alkyl, or —C(O)Oalkyl,
wherein if Y^{Fd} or Y^{Ff} is substituted with A², then Y^{Fe} is a bond, or a pharmaceutically acceptable salt thereof.

In some embodiments, the compound of Formula FB has the structure of Formula FB1:

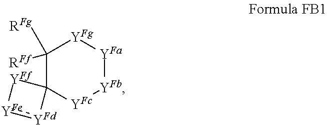

or a pharmaceutically acceptable salt thereof.

In some embodiments, the degradation moiety includes the structure of Formula F1:

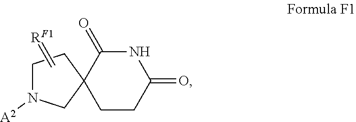

where A² is a bond between the degrader and the linker; and R^{F1} is absent or O, or a pharmaceutically acceptable salt thereof.

In some embodiments, R^{F1} is absent. In some embodiments, R^{F1} is O.

In some embodiments, the structure of Formula F1 is

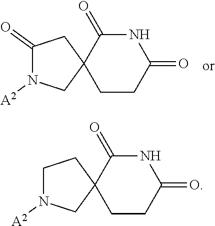

In some embodiments, the degradation moiety includes the structure Formula F2:

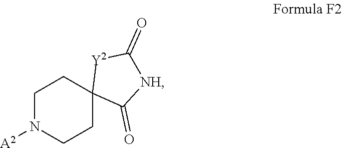

where A² is a bond between the degrader and the linker; and Y² is CH₂ or NH, or a pharmaceutically acceptable salt thereof.

In some embodiments, Y² is NH. In some embodiments, Y² is CH₂.

In some embodiments, structure of Formula F2 is

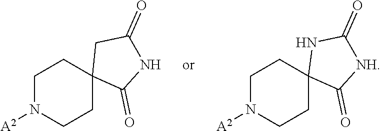

In some embodiments, the degradation moiety includes the structure Formula G:

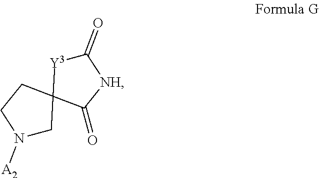

where A² is a bond between the degrader and the linker; and Y³ is CH₂ or NH, or a pharmaceutically acceptable salt thereof.

In some embodiments, Y³ is NH. In some embodiments, Y³ is CH₂.

In some embodiments, structure of Formula G is

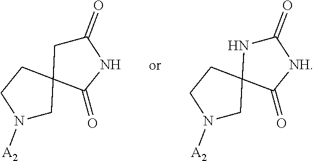

The degradation moiety may also include structures found in, e.g., WO2017/197036; WO2019/204354, WO2019/236483, WO2020/010177; and WO2020/010227, the structures of which are herein incorporated by reference.

In some embodiments, A hast the structure-of-Formula III:

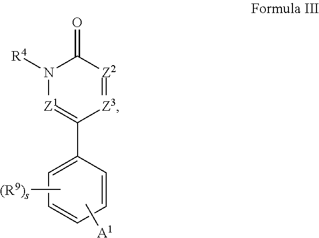

where
R⁴ is H, optionally substituted C₁-C₆ alkyl, optionally substituted C₂-C₆ alkenyl, optionally substituted C₁-C₆ heteroalkyl, or optionally substituted C₃-C₁₀ carbocyclyl; Z¹ is N or CR⁵;
Z² is N or CR^{6a};
Z³ is N or CR^{6b};
R⁵ is H, halogen, optionally substituted C₁-C₆ alkyl, optionally substituted C₁-C₆ heteroalkyl, optionally substituted C₃-C₁₀ carbocyclyl, or optionally substituted C₆-C₁₀ aryl;
R^{6a} is H, halogen, cyano, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; R^{6b} is H, halogen, cyano, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; or R^{6a} and R^{6b}, together with the carbon atoms to which each is attached, combine to form optionally substituted C₆-C₁₀ aryl or optionally substituted C₂-C₉ heteroaryl;
s is 0, 1, 2, 3, or 4;
each R⁹ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and
A¹ is a bond between A and the linker, or a pharmaceutically acceptable salt thereof.

In some embodiments, Z¹ is N. In some embodiments, Z¹ is CR⁵.

In some embodiments, Z² is N. In some embodiments, Z² is CR^{6a}.

In some embodiments, Z³ is N. In some embodiments, Z³ is CR^{6b}.

In some embodiments, Z¹ is CR⁵, Z² is CR^{6a}, and Z³ is CR^{6b}. In some embodiments, Z¹ is N, Z² is CR^{6a}, and Z² is CR^{6b}. In some embodiments, Z¹ is CR⁵, Z² is N, and Z³ is CR^{6b}. In some embodiments, Z¹ is N, Z² is CR^{6a}, and Z³ is N. In some embodiments, Z¹ is N, Z² is N, and Z³ is CR^{6b}. In some embodiments, Z¹ is CR⁵, Z² is N, and Z³ is N.

In some embodiments, R⁴ is H, optionally substituted C₁-C₆ alkyl, optionally substituted C₁-C₆ heteroalkyl, or optionally substituted C₃-C₁₀ carbocyclyl. In some embodiments, R⁴ is H, optionally substituted C₁-C₆ alkyl, optionally substituted C₂-C₆ alkenyl, or optionally substituted C₃-C₁₀ carbocyclyl. In some embodiments, R⁴ is H, optionally substituted C₁-C₆ alkyl, or optionally substituted C₃-C₁₀ carbocyclyl.

In some embodiments, optionally substituted C₁-C₆ alkyl is C₁-C₆ perfluoroalkyl.

In some embodiments, R⁴ is H,

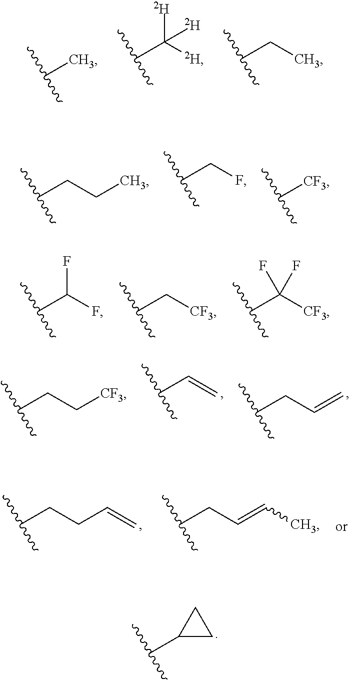

In some embodiments, R⁴ is

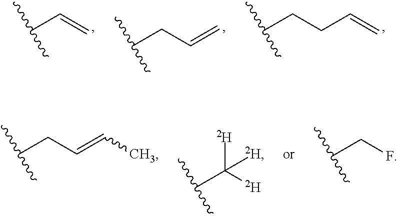

In some embodiments, R⁴ is H,

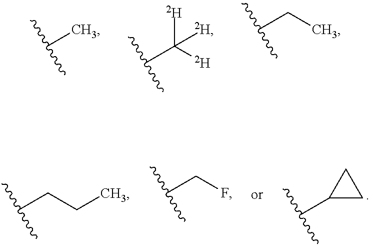

In some embodiments, R⁴ is H,

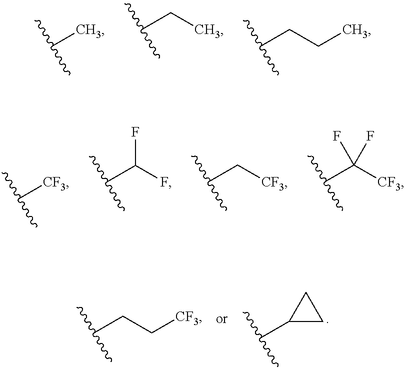

In some embodiments, R⁴ is H,

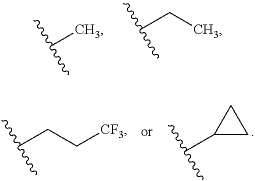

In some embodiments, R⁴ is H or

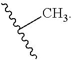

In some embodiments, R⁴ is H. In some embodiments, R⁴ is

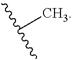

In some embodiments, R⁵ is H, optionally substituted C₁-C₆ alkyl, or optionally substituted C₆-C₁₀ aryl. In some embodiments, R⁵ is H, optionally substituted C₁-C₆ alkyl, optionally substituted C₁-C₆ heteroalkyl, or optionally substituted C₃-C₁₀ carbocyclyl. In some embodiments, R⁵ is H, optionally substituted C₁-C₆ alkyl, or optionally substituted C₃-C₁₀ carbocyclyl.

In some embodiments, optionally substituted C₁-C₆ alkyl is C₁-C₆ perfluoroalkyl.

In some embodiments, R⁵ is H,

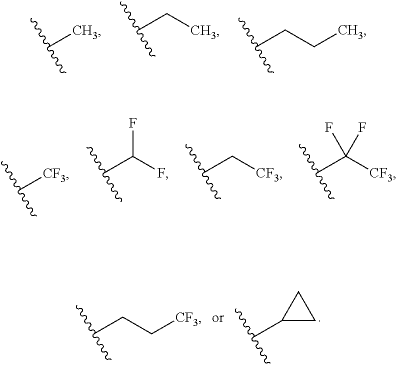

In some embodiments, R⁵ is H

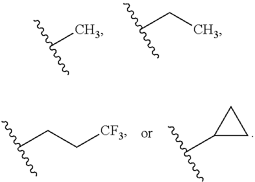

In some embodiments, R⁵ is H or

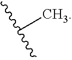

In some embodiments, R⁵ is H. In some embodiments, R⁵ is

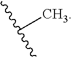

In some embodiments, R^{6a} is H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino.

In some embodiments, R^{6a} is H, halogen, cyano, optionally substituted C₁-C₆ alkyl, optionally substituted C₁-C₆ heteroalkyl, or optionally substituted C₃-C₁₀ carbocyclyl. In some embodiments, R^{6a} is H, halogen, cyano, optionally substituted C₁-C₆ alkyl, or optionally substituted C₁-C₆ heteroalkyl. In some embodiments, R^{6a} is H, halogen, cyano, or optionally substituted C₁-C₆ alkyl. In some embodiments, R^{6a} is optionally substituted C₁-C₆ heteroalkyl.

In some embodiments, R^{6a} is H, F, cyano,

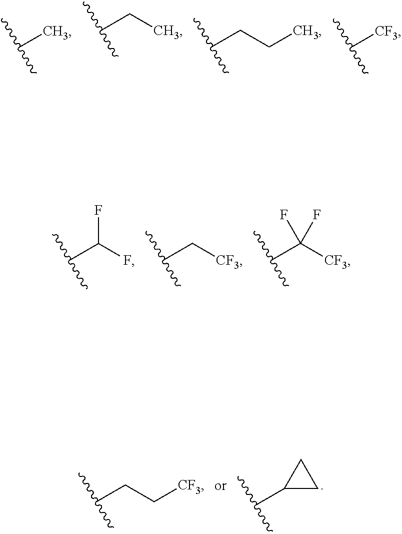

In some embodiments, R^{6a} is H, F, cyano,

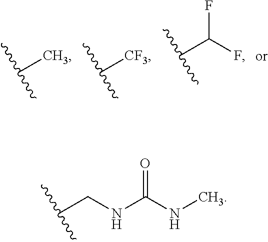

In some embodiments, R^{6a} is H, F, cyano, or

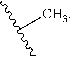

In some embodiments, R^{6a} is

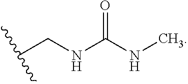 [SMILES: *CNC(=O)NC]

In some embodiments, R^{6a} is H or

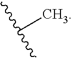

In some embodiments, R^{6a} is H. In some embodiments, R^{6a} is

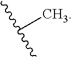

In some embodiments, R^{6b} is H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino.

In some embodiments, R^{6b} is H, halogen, cyano, optionally substituted C₁-C₆ alkyl, optionally substituted C₁-C₆ heteroalkyl, or optionally substituted C₃-C₁₀ carbocyclyl. In some embodiments, R^{6b} is H, halogen, cyano, optionally substituted C₁-C₆ alkyl, or optionally substituted C₁-C₆ heteroalkyl. In some embodiments, R^{6b} is H, halogen, cyano, or optionally substituted C₁-C₆ alkyl. In some embodiments, R^{6b} is optionally substituted C₁-C₆ heteroalkyl.

In some embodiments, R^{6b} is H, F, cyano,

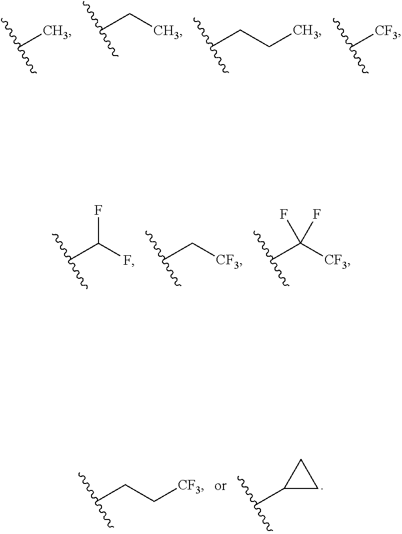

In some embodiments, R^{6b} is H, F, cyano,

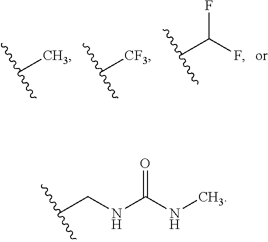

In some embodiments, R^{6b} is H, F, cyano, or

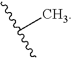

In some embodiments, R^{6b} is

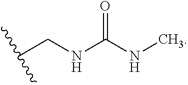 [SMILES: *CNC(=O)NC]

In some embodiments, R^{6b} is H or

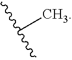

In some embodiments, R^{6b} is H. In some embodiments, R^{6b} is

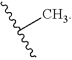

In some embodiments, R^{6a} and R^{6b}, together with the carbon atoms to which each is attached, combine to form optionally substituted C₆-C₁₀ aryl or optionally substituted C₂-C₉ heteroaryl.

In some embodiments, s is 0, 1, or 2. In some embodiments, s is 1 or 2. In some embodiments, s is 2.

In some embodiments, each R⁹ is, independently, halogen, optionally substituted C₁-C₆ alkyl, or optionally substituted C₁-C₆ heteroalkyl. In some embodiments, each R⁹ is, independently, optionally substituted C₁-C₆ alkyl or optionally substituted C₁-C₆ heteroalkyl.

In some embodiments, R⁹ is

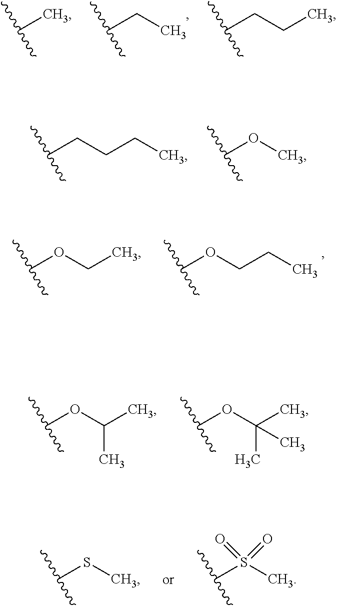

In some embodiments, each R⁹ is, independently, halogen,

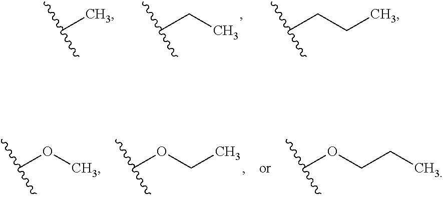

In some embodiments, each R⁹ is, independently, F, Cl,

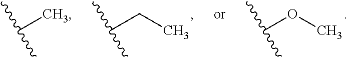

In some embodiments, the structure of Formula III has the structure of Formula IIIa:

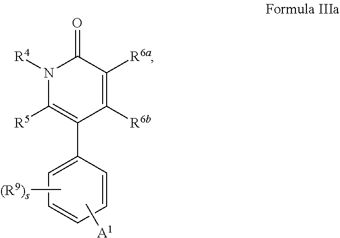

or a pharmaceutically acceptable salt thereof.

In some embodiments, the structure of Formula III has the structure of Formula IIIb:

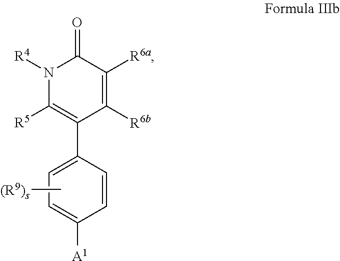

or a pharmaceutically acceptable salt thereof.

In some embodiments, the structure of Formula III has the structure of Formula Ilic:

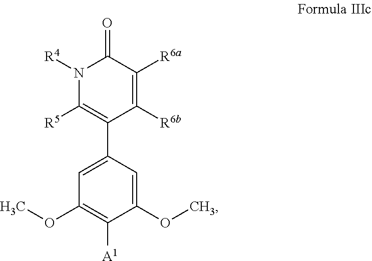

or a pharmaceutically acceptable salt thereof.

In some embodiments, the structure of Formula III has the structure of Formula IIId:

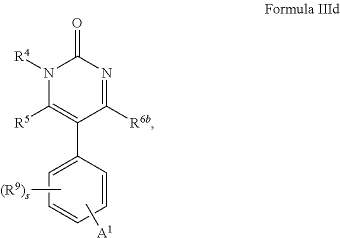

or a pharmaceutically acceptable salt thereof.

In some embodiments, the structure of Formula III has the structure of Formula IIIe:

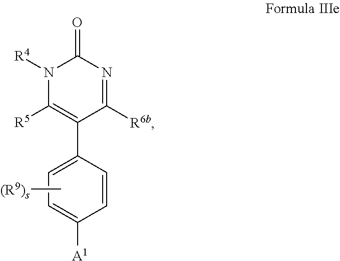

or a pharmaceutically acceptable salt thereof.

In some embodiments, the structure of Formula III has the structure of Formula IIIf:

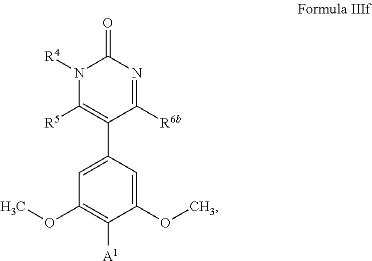

or a pharmaceutically acceptable salt thereof.

In some embodiments, the structure of Formula III has the structure of Formula IIg:

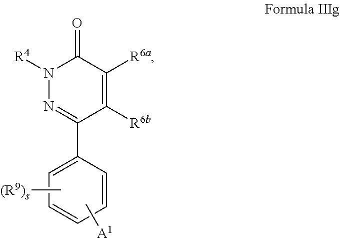

or a pharmaceutically acceptable salt thereof.

In some embodiments, the structure of Formula III has the structure of Formula IIIh:

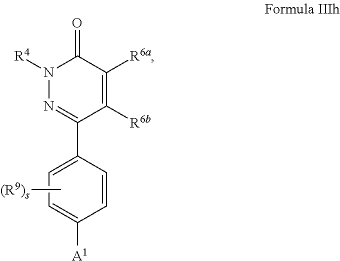

or a pharmaceutically acceptable salt thereof.

In some embodiments, the structure of Formula III has the structure of Formula IIIi:

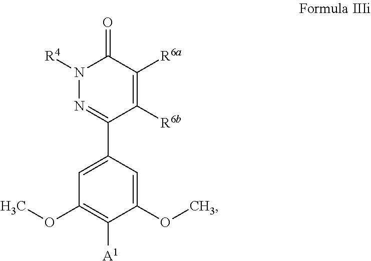

or a pharmaceutically acceptable salt thereof.

In some embodiments, the structure of Formula III has the structure of Formula IV:

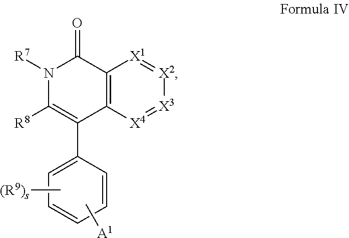

where
R⁷ is H, optionally substituted C₁-C₆ alkyl, optionally substituted C₂-C₆ alkenyl, optionally substituted C₁-C₆ heteroalkyl, or optionally substituted C₃-C₁₀ carbocyclyl;
R⁸ is H, halogen, optionally substituted C₁-C₆ alkyl, optionally substituted C₁-C₆ heteroalkyl, optionally substituted C₃-C₁₀ carbocyclyl, or optionally substituted C₆-C₁₀ aryl;
s is 0, 1, 2, 3, or 4;
each R⁹ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
X¹ is N or CR^{10a};
X² is N or CR^{10b};
X³ is N or CR^{10c};
X⁴ is N or CR^{10d};
each of R^{10a}, R^{10b}, R^{10c}, and R^{10d} is, independently, H, halogen, hydroxy, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and
A¹ is a bond between A and the linker, or a pharmaceutically acceptable salt thereof.

In some embodiments, X¹ is N. In some embodiments, X¹ is CR^{10a}. In some embodiments, X² is N. In some embodiments, X² is CR^{10b}. In some embodiments, X³ is N. In some embodiments, X³ is CR^{10c}. In some embodiments, X⁴ is N. In some embodiments, X¹ is CR^{10d}.

In some embodiments, X¹ is CR^{10a}, X² is CR^{10b}, X³ is CR^{10c}, and X⁴ is CR^{10d}. In some embodiments, X¹ is N, X² is CR^{10b}, X³ is CR^{10c}, and X⁴ is CR^{10d}. In some embodiments, X¹ is CR^{10a}, X² is N, X³ is CR^{10c}, and X⁴ is CR^{10d}. In some embodiments, X¹ is CR^{10a}, X² is CR^{10b}, X³ is N, and X⁴ is CR^{10d}. In some embodiments, X¹ is CR^{10a}, X² is CR^{10b}, X³ is CR^{10c}, and X⁴ is N. In some embodiments, X¹ is N, X² is N, X³ is CR^{10c}, and X⁴ is CR^{10d}. In some embodiments, X¹ is N, X² is CR^{10b}, X³ is N, and X⁴ is CR^{10d}. In some embodiments, X¹ is N, X² is CR^{10b}, X³ is CR^{10c}, and X⁴ is N. In some embodiments, X¹ is CR^{10a}X² is N, X³ is N, and X⁴ is CR^{10d}. In some embodiments, X¹ is CR^{10a}, X² is N, X³ is CR^{10c}, and X⁴ is N. In some embodiments, X¹ is CR^{10a}, X² is CR^{10b}, X³ is N, and X⁴ is N.

In some embodiments, R⁷ is H, optionally substituted C₁-C₆ alkyl, optionally substituted C₁-C₆ heteroalkyl, or optionally substituted C₃-C₁₀ carbocyclyl. In some embodiments, R⁷ is H, optionally substituted C₁-C₆ alkyl, optionally substituted C₂-C₆ alkenyl, or optionally substituted C₃-C₁₀ carbocyclyl. In some embodiments, R⁷ is H, optionally substituted C₁-C₆ alkyl, or optionally substituted C₃-C₁₀ carbocyclyl.

In some embodiments, optionally substituted C₁-C₆ alkyl is C₁-C₆ perfluoroalkyl.

In some embodiments, R⁷ is H,

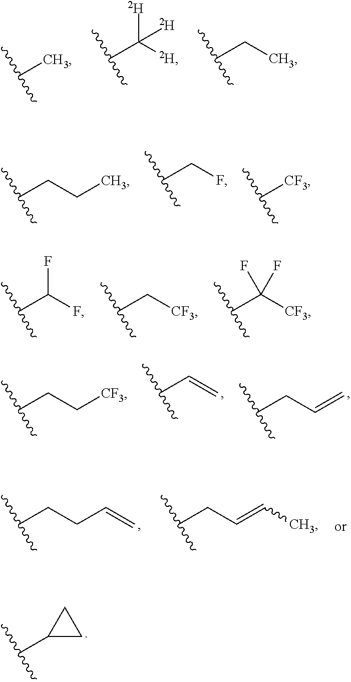

In some embodiments, R⁷ is H,

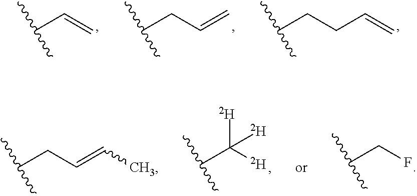

In some embodiments, R⁷ is H,

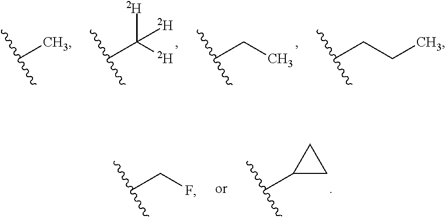

In some embodiments, R⁷ is H,

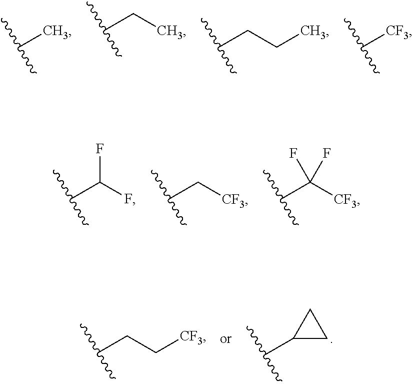

In some embodiments, R⁷ is H,

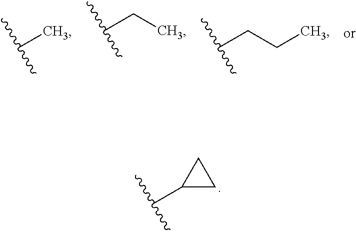

In some embodiments, R⁷ is H or

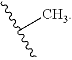

In some embodiments, R⁷ is H. In some embodiments, R⁷ is

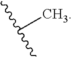

In some embodiments, R⁸ is H, optionally substituted C₁-C₆ alkyl, or optionally substituted C₆-C₁₀ aryl. In some embodiments, R⁸ is H, optionally substituted C₁-C₆ alkyl, optionally substituted C₁-C₆ heteroalkyl, or optionally substituted C₃-C₁₀ carbocyclyl. In some embodiments, R⁸ is H, optionally substituted C₁-C₆ alkyl, or optionally substituted C₃-C₁₀ carbocyclyl. In some embodiments, R⁸ is H or optionally substituted C₁-C₆ alkyl.

In some embodiments, optionally substituted C₁-C₆ alkyl is C₁-C₆ perfluoroalkyl.

In some embodiments, R⁸ is H,

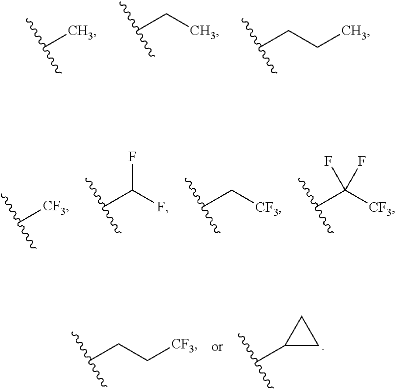

In some embodiments, R⁸ is H,

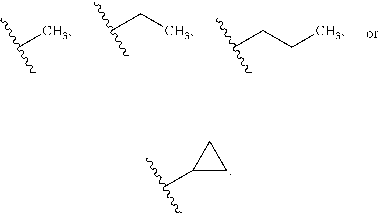

In some embodiments, R⁸ is H or

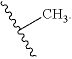

In some embodiments, R⁸ is H. In some embodiments, R⁸ is

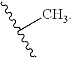

In some embodiments, s is 0, 1, or 2. In some embodiments, s is 1 or 2. In some embodiments, s is 2. In some embodiments, s is 1.

In some embodiments, each R⁹ is, independently, halogen, optionally substituted C₁-C₆ alkyl, or optionally substituted C₁-C₆ heteroalkyl. In some embodiments, each R⁹ is, independently, optionally substituted C₁-C₆ alkyl or optionally substituted C₁-C₆ heteroalkyl.

In some embodiments, R⁹ is

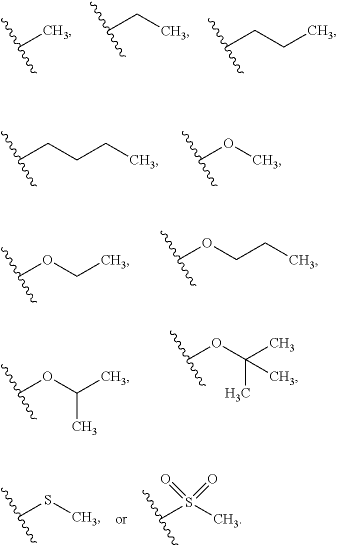

In some embodiments, each R⁹ is, independently, halogen,

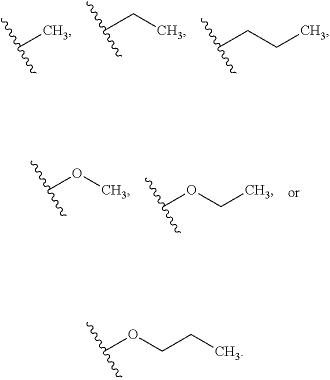

In some embodiments, each R⁹ is, independently, F, Cl,

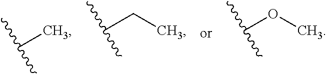

In some embodiments, R^{10a} is H, halogen, cyano, optionally substituted C₁-C₆ alkyl, optionally substituted C₁-C₆ heteroalkyl, or optionally substituted C₃-C₁₀ carbocyclyl. In some embodiments, R^{10a} is H, halogen, cyano, optionally substituted C₁-C₆ alkyl, or optionally substituted C₁-C₆ heteroalkyl. In some embodiments, R^{10a} is H, halogen, cyano, or optionally substituted C₁-C₆ alkyl. In some embodiments, R^{10a} is optionally substituted C₁-C₆ heteroalkyl.

In some embodiments, R^{10a} is H, F, cyano,

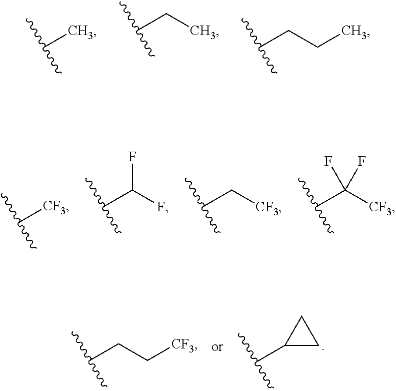

In some embodiments, R^{10a} is H, F, cyano,

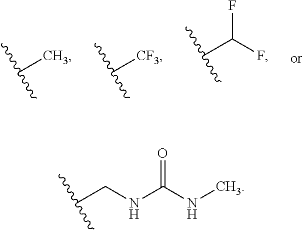

In some embodiments, R^{10a} is H, F, cyano, or

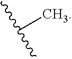

In some embodiments, R^{10a} is

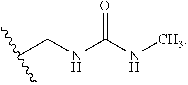 [SMILES: *CNC(=O)NC]

In some embodiments, R^{10a} is H or

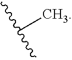

In some embodiments, R^{10a} is H. In some embodiments, R^{10a} is

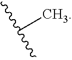

In some embodiments, R^{10b} is H, halogen, cyano, optionally substituted C₁-C₆alkyl, optionally substituted C₁-C₆ heteroalkyl, or optionally substituted C₃-C₁₀ carbocyclyl. In some embodiments, R^{10b} is H, halogen, cyano, optionally substituted C₁-C₆ alkyl, or optionally substituted C₁-C₆ heteroalkyl. In some embodiments, R^{10b} is H, halogen, cyano, or optionally substituted C₁-C₆ alkyl. In some embodiments, R^{10b} is optionally substituted C₁-C₆ heteroalkyl.

In some embodiments, R^{10b} is H, F, cyano,

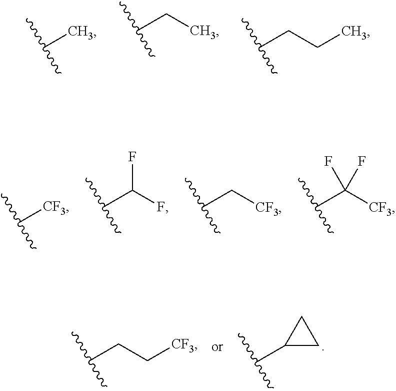

In some embodiments, R^{10b} is H, F, cyano,

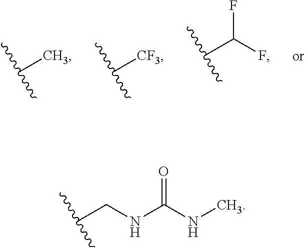

In some embodiments, R^{10b} is H, F, cyano, or

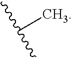

In some embodiments, R^{10b} is

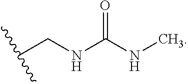 [SMILES: *CNC(=O)NC]

In some embodiments, R^{10b} is H or

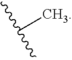

In some embodiments, R^{10b} is H. In some embodiments, R^{10b} is

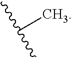

In some embodiments, R^{10c} is H, halogen, cyano, optionally substituted C₁-C₆ alkyl, optionally substituted C₁-C₆ heteroalkyl, or optionally substituted C₃-C₁₀ carbocyclyl. In some embodiments, R^{10c} is H, halogen, cyano, optionally substituted C₁-C₆ alkyl, or optionally substituted C₁-C₆ heteroalkyl. In some embodiments, R^{10c} is H, halogen, cyano, or optionally substituted C₁-C₆ alkyl. In some embodiments, R^{10c} is optionally substituted C₁-C₆ heteroalkyl.

In some embodiments, R^{10c} is H, F, cyano,

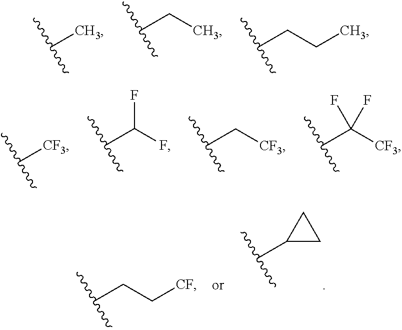

In some embodiments, R^{10c} is H, F, cyano,

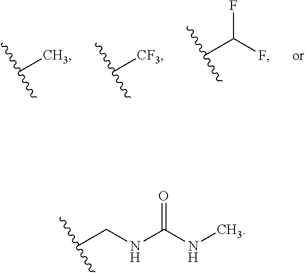

In some embodiments, R^{10c} is H, F, cyano, or

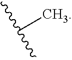

In some embodiments, R^{10c} is

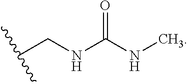 [SMILES: *CNC(=O)NC]

In some embodiments, R^{10c} is H or

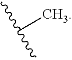

In some embodiments, R^{10c} is H. In some embodiments, R^{10c} is

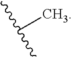

In some embodiments, R^{10d} is H, halogen, cyano, optionally substituted C₁-C₆alkyl, optionally substituted C₁-C₆ heteroalkyl, or optionally substituted C₃-C₁₀ carbocyclyl. In some embodiments, R^{10d} is H, halogen, cyano, optionally substituted C₁-C₆ alkyl, or optionally substituted C₁-C₆ heteroalkyl. In some embodiments, R^{10d} is H, halogen, cyano, or optionally substituted C₁-C₆ alkyl. In some embodiments, R^{10d} is optionally substituted C₁-C₆ heteroalkyl.

In some embodiments, R^{10d} is H, F, cyano,

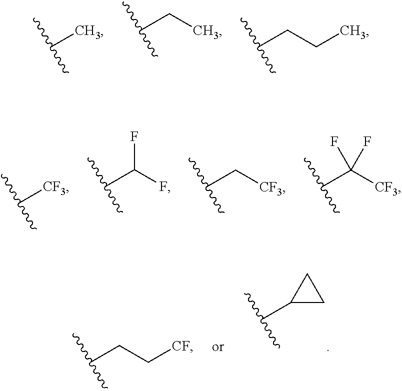

In some embodiments, R^{10d} is H, F, cyano,

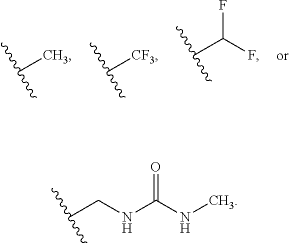

In some embodiments, R^{10d} is H, F, cyano, or

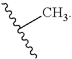

In some embodiments, R^{10d} is

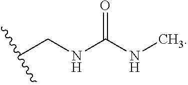 [SMILES: *CNC(=O)NC]

In some embodiments, R^{10d} is H or

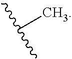

In some embodiments, R^{10d} is H. In some embodiments, R^{10d} is

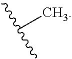

In some embodiments, each of R^{10a}, R^{10b}, R^{10c}, and R^{10d} is, independently, optionally substituted C₁-C₆ alkyl, optionally substituted C₁-C₆ heteroalkyl, or optionally substituted amino.

In some embodiments, each of R^{10a}, R^{10b}, R^{10c}, and R^{10d} is, independently, —NH₂,

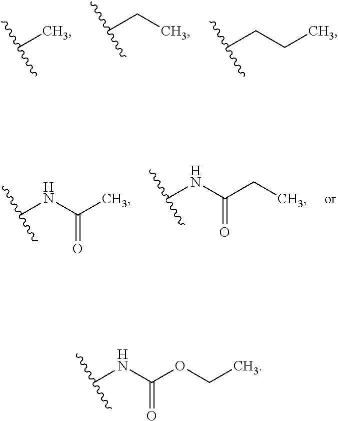

In some embodiments, A includes the structure of Formula IVa:

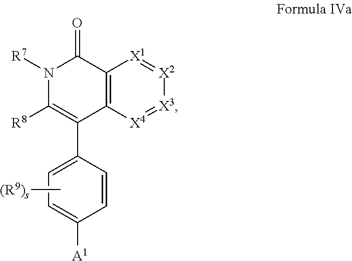

or a pharmaceutically acceptable salt thereof.

In some embodiments, A includes the structure of Formula IVb:

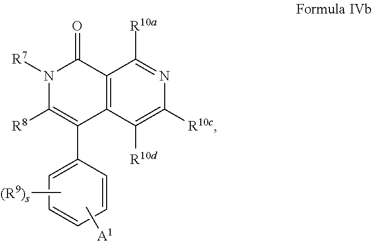

or a pharmaceutically acceptable salt thereof.

In some embodiments, A includes the structure of Formula IVc:

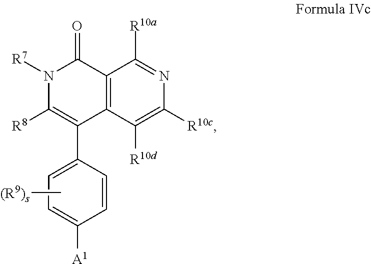

or a pharmaceutically acceptable salt thereof.

In some embodiments, A includes the structure of Formula IVd:

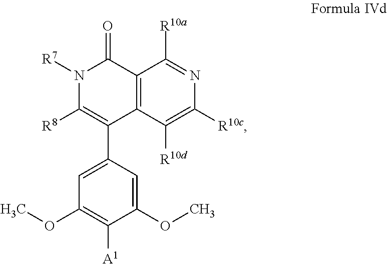

or a pharmaceutically acceptable salt thereof.

In some embodiments, A includes the structure of Formula IVe:

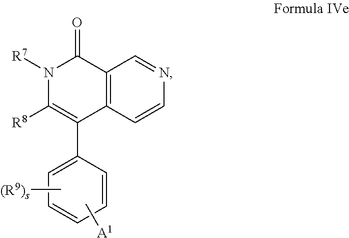

or a pharmaceutically acceptable salt thereof.

In some embodiments, A includes the structure of Formula IVf:

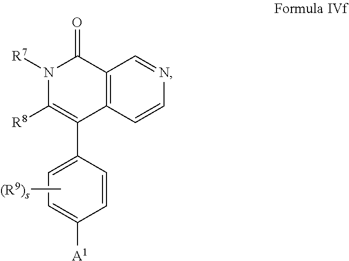

or a pharmaceutically acceptable salt thereof.

In some embodiments, A includes the structure of Formula IVg:

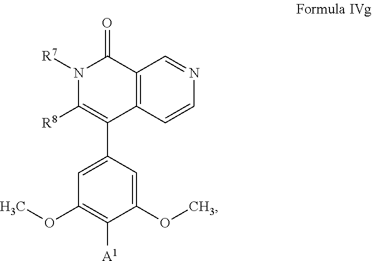

or a pharmaceutically acceptable salt thereof.

In some embodiments, A includes the structure of Formula IVh:

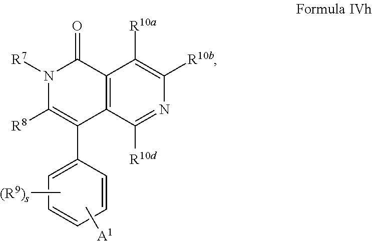

or a pharmaceutically acceptable salt thereof.

In some embodiments, A includes the structure of Formula IVi:

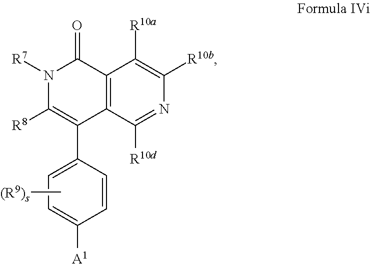

or a pharmaceutically acceptable salt thereof.

In some embodiments, A includes the structure of Formula IVj:

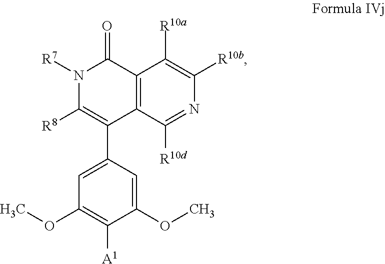

or a pharmaceutically acceptable salt thereof.

In some embodiments, A includes the structure of Formula IVk:

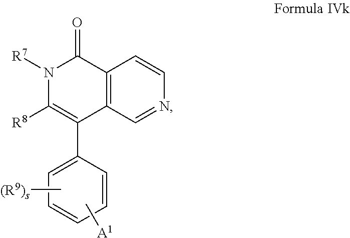

or a pharmaceutically acceptable salt thereof.

In some embodiments, A includes the structure of Formula IVm:

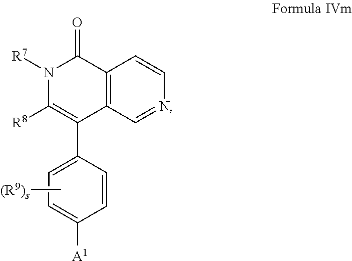

or a pharmaceutically acceptable salt thereof.

In some embodiments, A includes the structure of Formula IVn:

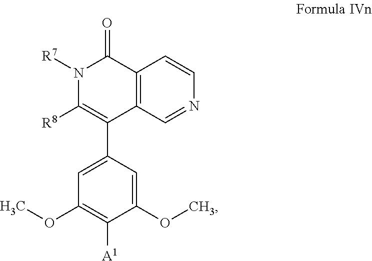

or a pharmaceutically acceptable salt thereof.

In some embodiments, A includes the structure of any one of

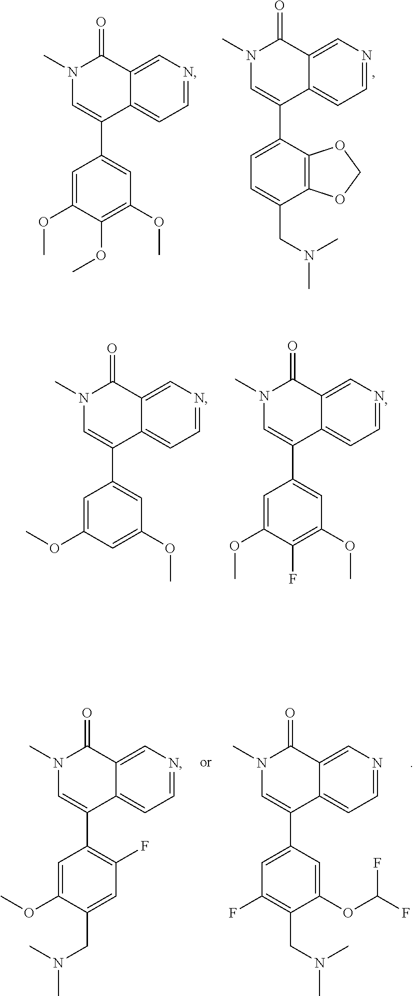

In some embodiments, A includes the structure of Formula V

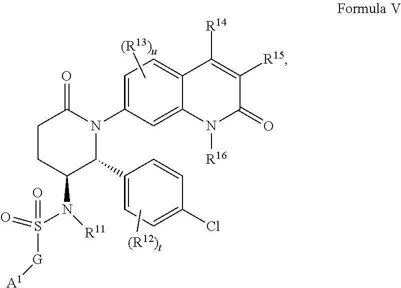

where
each R¹¹ and R¹⁶ is, independently, H, optionally substituted C₁-C₆ alkyl, or optionally substituted C₁-C₆ heteroalkyl;
t is 0, 1, 2, 3, or 4;
each R¹² is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
u is 0, 1, 2, 3, or 4;
each R¹³ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each R¹⁴ and R¹⁵ is, independently, selected form the group consisting of H, halogen, optionally substituted C₁-C₆ alkyl, or optionally substituted C₆-C₁₀ aryl; G is optionally substituted C₁-C₆ alkylene, optionally substituted C₆-C₁₀ arylene, or optionally substituted C₃-C₆ carbocyclylene; and
A¹ is a bond between A and the linker, or a pharmaceutically acceptable salt thereof.

In some embodiments, A includes the structure of Formula VI:

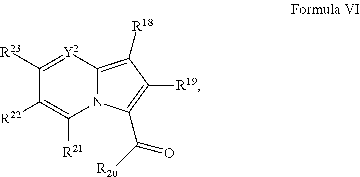

where
Y² is CR¹⁷ or N;
R¹⁸ is A¹, optionally substituted C₆-C₁₀ aryl or C₂-C₉ heteroaryl;
R¹⁹ is H, halogen, optionally substituted C₁-C₆ alkyl, or optionally substituted C₆-C₁₀ aryl;
R²⁰ is H, optionally substituted C₁-C₆ alkyl, or optionally substituted C₆-C₁₀ aryl;
each R¹⁷, R²¹, and R²² is, independently, H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
R²³ is H or —NR²⁴R²⁵; and
each of R²⁴ and R²⁵ is, independently, H, A¹, optionally substituted C₁-C₆ alkyl, or optionally substituted C₁-C₆ heteroalkyl, or R²⁴ and R²⁵ combine to form optionally substituted C₂-C₉ heterocyclyl,
where one of R¹⁸, R²⁴, or R²⁵ is A¹, or a pharmaceutically acceptable salt thereof.

In some embodiments, A includes the structure of Formula VII:

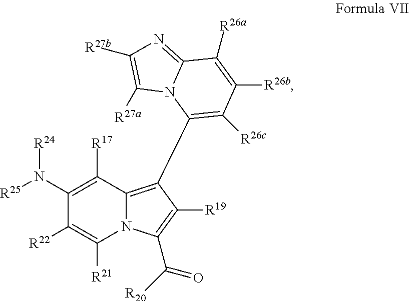

where
each R^{26a}, R^{26b}, and R^{26c} is, independently, H, A¹, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each R^{27a} and R^{27b} is, independently, H, halogen, optionally substituted C₁-C₆ alkyl, or optionally substituted C₆-C₁₀ aryl;
R¹⁹ is H, halogen, optionally substituted C₁-C₆ alkyl, or optionally substituted C₆-C₁₀ aryl;
R²⁰ is H, optionally substituted C₁-C₆ alkyl, or optionally substituted C₆-C₁₀ aryl;
each R¹⁷, R²¹, and R²² is, independently, H, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and
each of R²⁴ and R²⁵ is, independently, H, A¹, optionally substituted C₁-C₆ alkyl, or optionally substituted C₁-C₆ heteroalkyl, or R²⁴ and R²⁵ combine to form optionally substituted C₂-C₉ heterocyclyl,
where one of R^{26a}, R^{26b}, R^{26c}, R²⁴, or R²⁵ is A¹, or a pharmaceutically acceptable salt thereof.

In some embodiments, A includes the structure of Formula VIII:

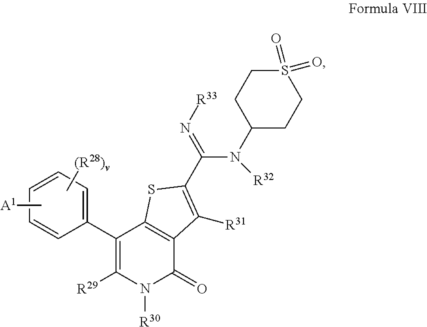

where
v is 0, 1, 2, 3, or 4;
each R²⁸ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
R²⁹ is H, halogen, optionally substituted C₁-C₆ alkyl, or optionally substituted C₆-C₁₀ aryl;
R³¹ is H, halogen, optionally substituted C₁-C₆ alkyl, or optionally substituted C₆-C₁₀ aryl;
each R³⁰, R³², and R³³ is, independently, H, optionally substituted C₁-C₆ alkyl, or optionally substituted C₁-C₆ heteroalkyl; and
A¹ is a bond between A and the linker, or a pharmaceutically acceptable salt thereof.

In some embodiments, A includes the structure of Formula IX:

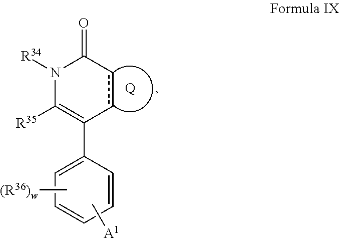

where

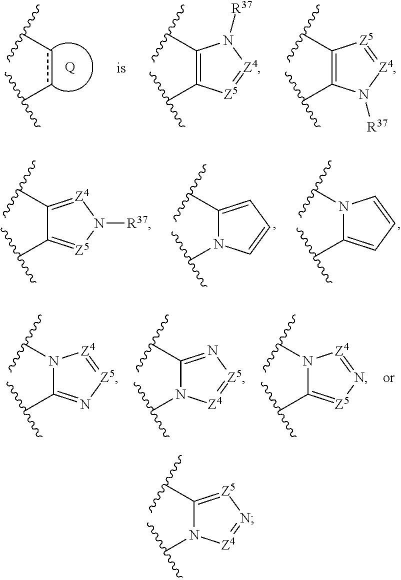

Z⁴ is N or CR³⁸;
Z⁵ is N or CR³⁹;
R³⁴ is H, optionally substituted C₁-C₆ alkyl, optionally substituted C₂-C₆ alkenyl, optionally substituted C₁-C₆ heteroalkyl, or optionally substituted C₃-C₁₀ carbocyclyl;
R³⁵ is H, halogen, optionally substituted C₁-C₆ alkyl, optionally substituted C₁-C₆ heteroalkyl, optionally substituted C₃-C₆ carbocyclyl, or optionally substituted C₆-C₁₀ aryl;
R³⁷ is H, optionally substituted C₁-C₆ alkyl, or optionally substituted C₁-C₆ heteroalkyl;
R³⁸ is H, halogen, optionally substituted C₁-C₆ alkyl, or optionally substituted C₆-C₁₀ aryl;
R³⁹ is H, halogen, optionally substituted C₁-C₆ alkyl, or optionally substituted C₆-C₁₀ aryl;
w is 0, 1, 2, 3, or 4;
each R³⁶ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and
A¹ is a bond between A and the linker, or a pharmaceutically acceptable salt thereof.

In some embodiments, Z⁴ is N. In some embodiments, Z⁴ is R³⁸. In some embodiments, Z⁵ is N.

In some embodiments, Z⁵ is R³⁹.

In some embodiments, Z⁴ is N and Z⁵ is R³⁹. In some embodiments, Z⁴ is R³⁸ and Z⁵ is N. In some embodiments, Z⁴ is R³⁸ and Z⁵ is R³⁹.

In some embodiments,

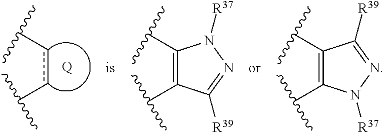

In some embodiments,

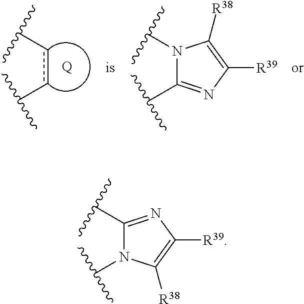

In some embodiments, is

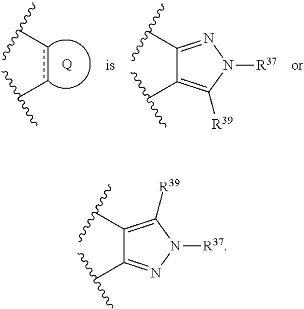

In some embodiments,

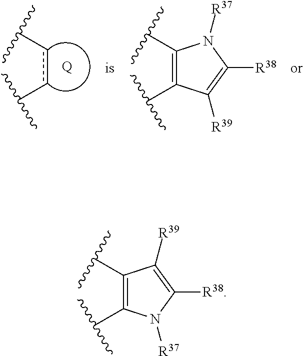

In some embodiments,

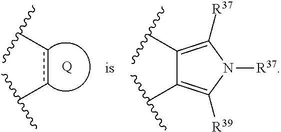

In some embodiments is

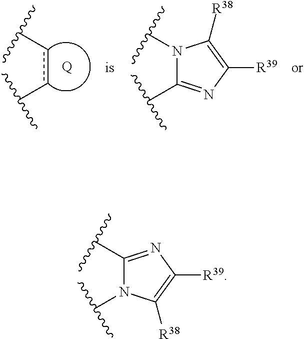

In some embodiments

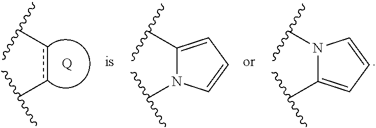

In some embodiments, R³⁷ is H or optionally substituted C₁-C₆ alkyl. In some embodiments, R³⁷ is H or

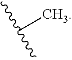

In some embodiments, R³⁸ is H or optionally substituted C₁-C₆ alkyl. In some embodiments, R³⁸ is H or

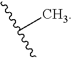

In some embodiments, R³⁹ is H or optionally substituted C₁-C₆ alkyl. In some embodiments, R³⁹ is H or

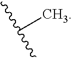

In some embodiments, R³⁴ is H, optionally substituted C₁-C₆ alkyl, optionally substituted C₁-C₆ heteroalkyl, or optionally substituted C₃-C₁₀ carbocyclyl. In some embodiments, R³⁴ is H, optionally substituted C₁-C₆ alkyl, optionally substituted C₂-C₆ alkenyl, or optionally substituted C₃-C₁₀ carbocyclyl. In some embodiments, R³⁴ is H, optionally substituted C₁-C₆ alkyl, or optionally substituted C₃-C₁₀ carbocyclyl.

In some embodiments, optionally substituted C₁-C₆ alkyl is C₁-C₆ perfluoroalkyl.

In some embodiments, R³⁴ is H,

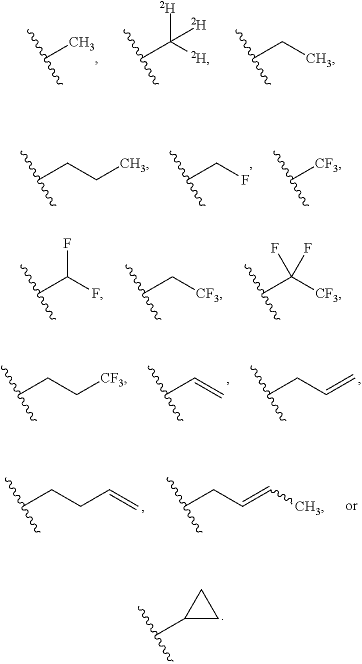

In some embodiments, R³⁴ is

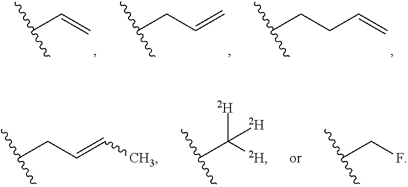

In some embodiments, R³⁴ is H,

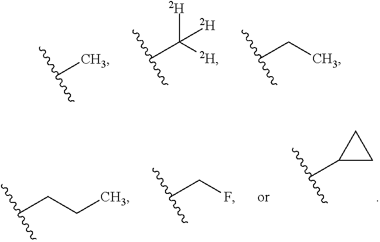

In some embodiments, R³⁴ is H,

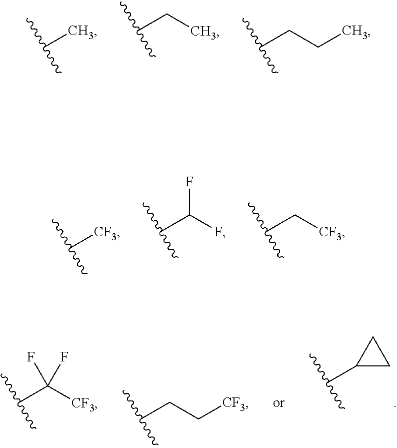

In some embodiments, R³⁴ is H,

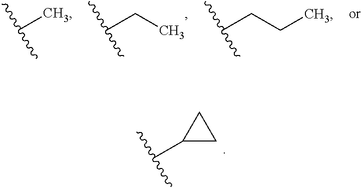

In some embodiments, R³⁴ is H or

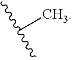

In some embodiments, R³⁴ is H. In some embodiments, R³⁴ is

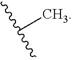

In some embodiments, R³⁵ is H, optionally substituted C₁-C₆ alkyl, or optionally substituted C₆-C₁₀ aryl. In some embodiments, R³⁵ is H, optionally substituted C₁-C₆ alkyl, optionally substituted C₁-C₆ heteroalkyl, or optionally substituted C₃-C₁₀ carbocyclyl. In some embodiments, R³⁵ is H, optionally substituted C₁-C₆ alkyl, or optionally substituted C₃-C₁₀ carbocyclyl. In some embodiments, R³⁵ is H or optionally substituted C₁-C₆ alkyl.

In some embodiments, optionally substituted C₁-C₆ alkyl is C₁-C₆ perfluoroalkyl.

In some embodiments, R³⁵ is H,

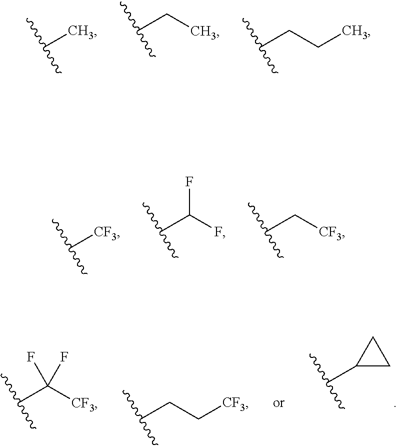

In some embodiments, R³⁵ is H,

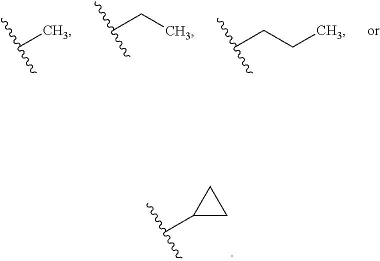

In some embodiments, R³⁵ is H or

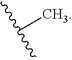

In some embodiments, R³⁵ is H. In some embodiments, R³⁵ is

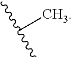

In some embodiments, w is 0, 1, or 2. In some embodiments, w is 1 or 2. In some embodiments, w is 2.

In some embodiments, each R³⁶ is, independently, halogen, optionally substituted C₁-C₆ alkyl, or optionally substituted C₁-C₆ heteroalkyl. In some embodiments, each R³⁶ is, independently, optionally substituted C₁-C₆ alkyl or optionally substituted C₁-C₆ heteroalkyl.

In some embodiments, each R³⁶ is, independently,

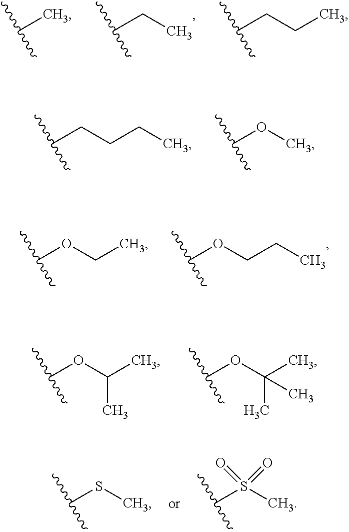

In some embodiments, each R³⁶ is, independently, halogen,

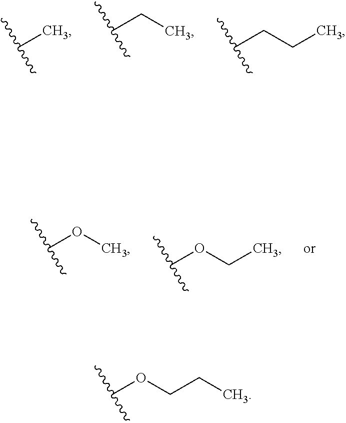

In some embodiments, each R³⁶ is, independently, F, Cl,

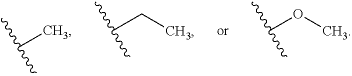

In some embodiments, the structure of Formula IX has the structure of Formula IXa:

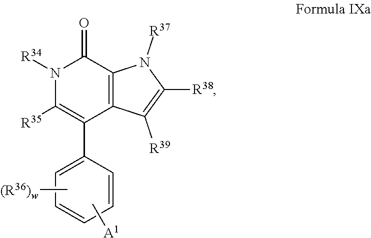

or a pharmaceutically acceptable salt thereof.

In some embodiments, the structure of Formula IX has the structure of Formula IXb:

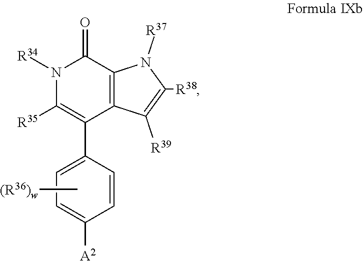

or a pharmaceutically acceptable salt thereof.

In some embodiments, the structure of Formula IX has the structure of Formula IXc:

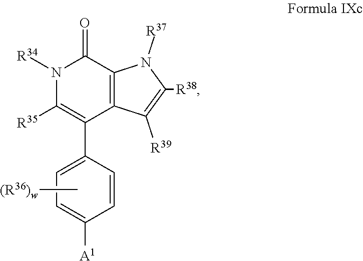

or a pharmaceutically acceptable salt thereof.

In some embodiments, the structure of Formula IX has the structure of Formula IXd:

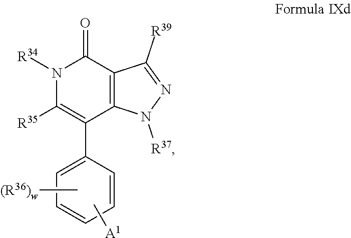

or a pharmaceutically acceptable salt thereof.

In some embodiments, the structure of Formula IX has the structure of Formula IXe:

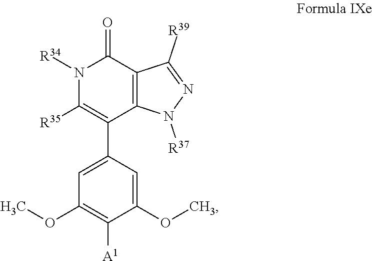

or a pharmaceutically acceptable salt thereof.

In some embodiments, the structure of Formula IX has the structure of Formula IXf:

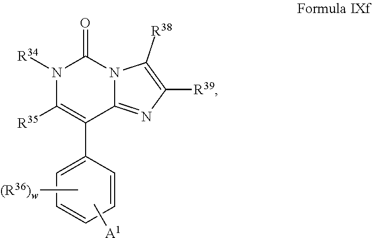

or a pharmaceutically acceptable salt thereof.

In some embodiments, the structure of Formula IX has the structure of Formula IXg:

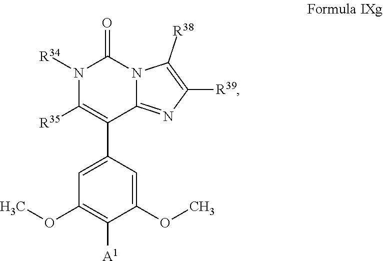

or a pharmaceutically acceptable salt thereof.

In some embodiments, the structure of Formula IX has the structure of Formula IXh:

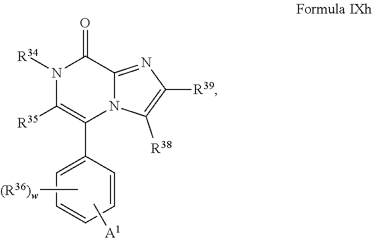

or a pharmaceutically acceptable salt thereof.

In some embodiments, the structure of Formula IX has the structure of Formula IXi:

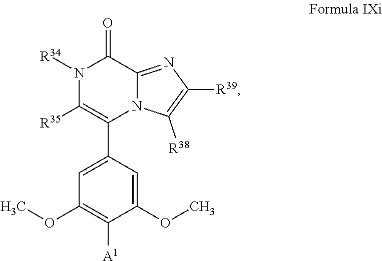

or a pharmaceutically acceptable salt thereof.

In some embodiments, A includes the structure of:

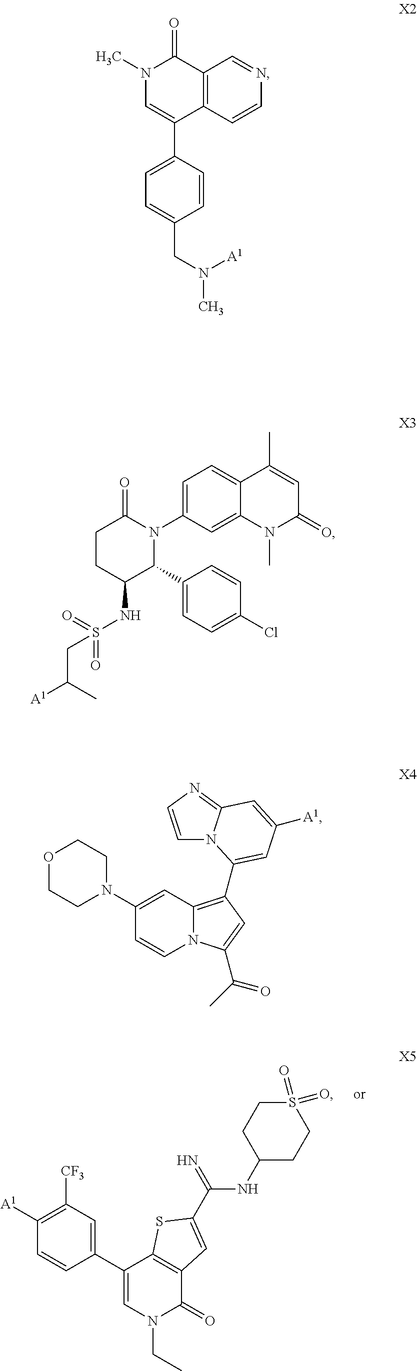

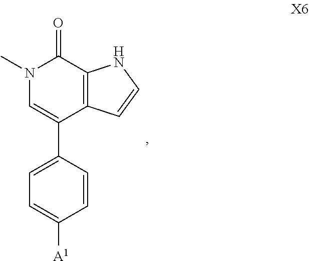

where A¹ is a bond between A and the linker, or derivative or analog thereof.

In some embodiments, the compound has the structure of any one of compounds D1-D177 in Table 1A, or a pharmaceutically acceptable salt thereof. In some embodiments, the compound has the structure of any one of compounds D178-D371 in Table 1B, or a pharmaceutically acceptable salt thereof. In some embodiments, the compound has the structure of any one of compounds D372-D476 in Table 1 D, or a pharmaceutically acceptable salt thereof.

In some embodiments, the compound has the structure of any one of compounds D1, D3, D6, D9-D20, D23, D33, D33-D35, D37-D40, D42, D44-D47, D50-D53, D56-D60, D67, D69, D71-D73, D75, D76, D80, D81, D89, D92, D100, D108, D113, D122-D124, D128-D132, D143, D152, D157, D167, D168, D170, D171, D173, and D176 in Table 1A, or a pharmaceutically acceptable salt thereof. In some embodiments, the compound has the structure of any one of compounds D178, D180, D184-D189, D191, D194, D197-D199, D201-D208, D211, D213-D230, D235-D244, D246, D247, D250-D263, D268, D269, D271-D275, D277, D279, D280, D287-D291, D297-D299, D300-D302, D304, D306-D308, D310, D312, D313, D315, D316, D318-D333, D335-D341, D343-D349, D353, D354, D356-D363, and D366-D371 in Table 1 B, or a pharmaceutically acceptable salt thereof. In some embodiments, the compound has the structure of any one of compounds D372-D379, D381, D382, D384-D388, D395-D428, D430, D431, D433, D434, D436, D438-D444, D448, D450, D453-D460, D462, D463, D465, D466, D471, and D476 in Table 1 D, or a pharmaceutically acceptable salt thereof.

In an aspect, the disclosure features a compound having the structure of any one of compounds D1-D177 in Table 1A, or a pharmaceutically acceptable salt thereof.

In another aspect, the disclosure features a compound having the structure of any one of compounds D178-D371 in Table 1 B, or a pharmaceutically acceptable salt thereof.

In another aspect, the disclosure features a compound having the structure of any one of compounds D372-D476 in Table 1D, or a pharmaceutically acceptable salt thereof.

In another aspect, the disclosure features a compound having the structure of any one of compounds DD1-DD10 in Table 1C, or a pharmaceutically acceptable salt thereof.

In another aspect, the disclosure features a compound having the structure of any one of compounds DD11-DD16 in Table 1E, or a pharmaceutically acceptable salt thereof.

TABLE 1A
Compounds D1-D177 of the Disclosure

| Com- | |
|---|---|
| pound | |
| No. | Structure |
| D1 | 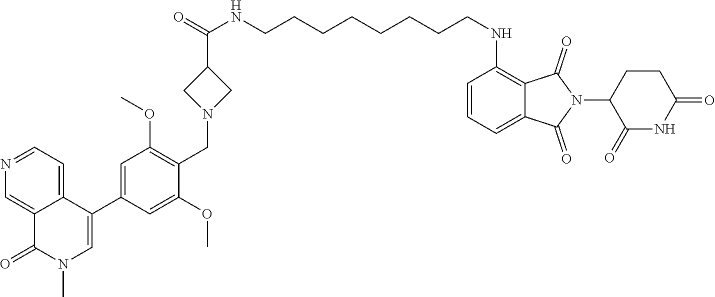 [SMILES: COc1cc(-c2cn(C)c(=O)c3cnccc23)cc(OC)c1CN1CC(C(=O)NCCCCCCCCNc2cccc3c2C(=O)N(C2CCC(=O)NC2=O)C3=O)C1] |
| D2 | 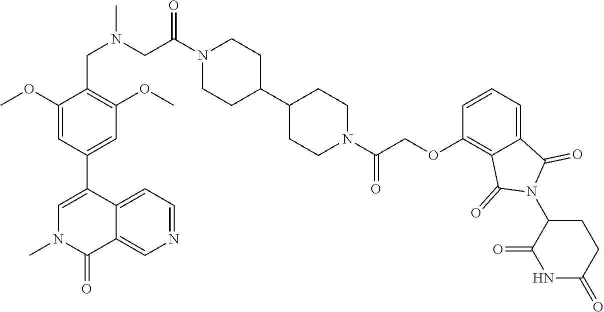 [SMILES: COc1cc(-c2cn(C)c(=O)c3cnccc23)cc(OC)c1CN(C)CC(=O)N1CCC(C2CCN(C(=O)COc3cccc4c3C(=O)N(C3CCC(=O)NC3=O)C4=O)CC2)CC1] |
| D3 | 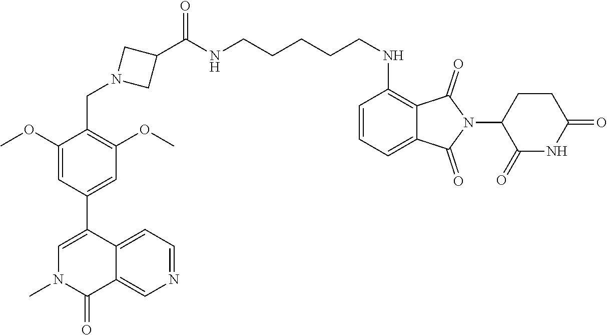 [SMILES: COc1cc(-c2cn(C)c(=O)c3cnccc23)cc(OC)c1CN1CC(C(=O)NCCCCCNc2cccc3c2C(=O)N(C2CCC(=O)NC2=O)C3=O)C1] |
| D4 | 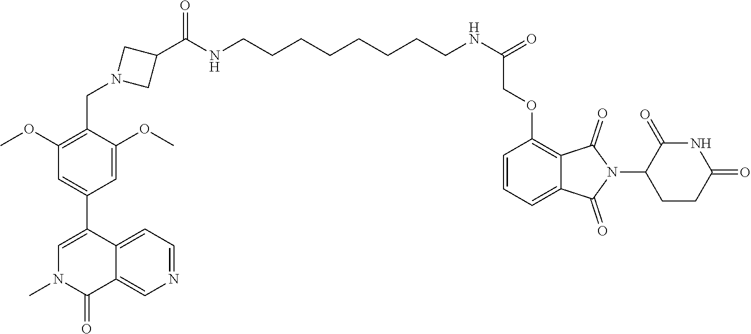 [SMILES: COc1cc(-c2cn(C)c(=O)c3cnccc23)cc(OC)c1CN1CC(C(=O)NCCCCCCCCNC(=O)COc2cccc3c2C(=O)N(C2CCC(=O)NC2=O)C3=O)C1] |
| D5 | 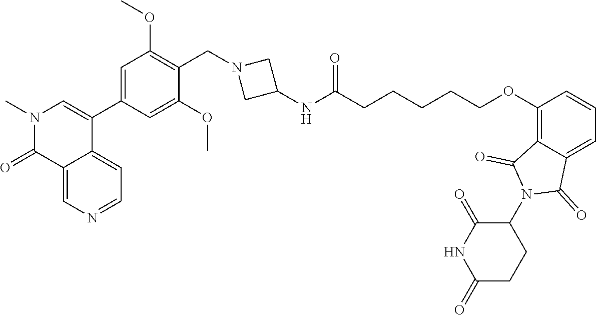 [SMILES: COc1cc(-c2cn(C)c(=O)c3cnccc23)cc(OC)c1CN1CC(NC(=O)CCCCCOc2cccc3c2C(=O)N(C2CCC(=O)NC2=O)C3=O)C1] |
| D6 | 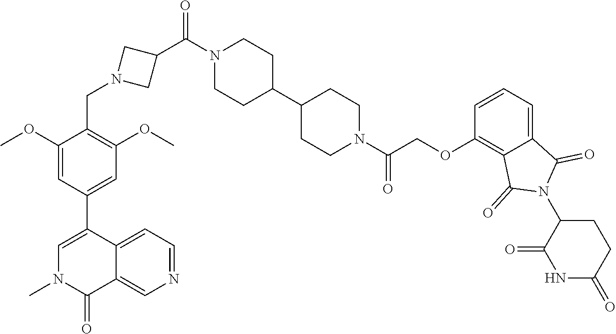 [SMILES: COc1cc(-c2cn(C)c(=O)c3cnccc23)cc(OC)c1CN1CC(C(=O)N2CCC(C3CCN(C(=O)COc4cccc5c4C(=O)N(C4CCC(=O)NC4=O)C5=O)CC3)CC2)C1] |
| D7 | 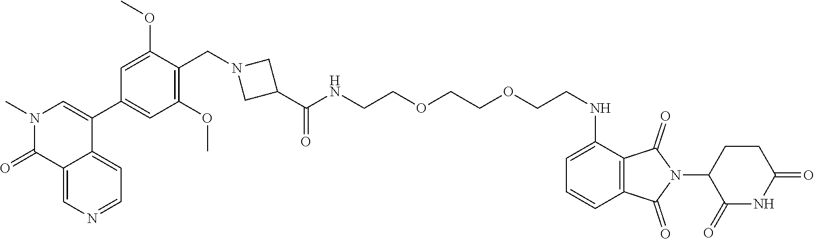 [SMILES: COc1cc(-c2cn(C)c(=O)c3cnccc23)cc(OC)c1CN1CC(C(=O)NCCOCCOCCNc2cccc3c2C(=O)N(C2CCC(=O)NC2=O)C3=O)C1] |
| D8 | 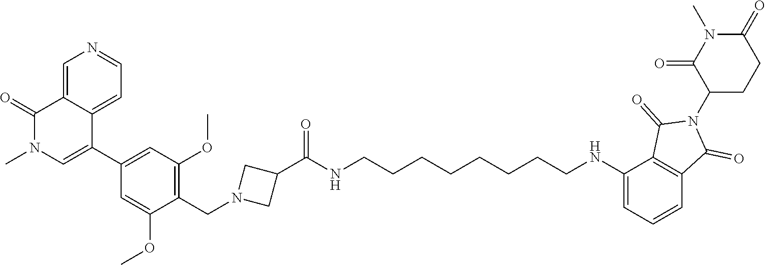 [SMILES: COc1cc(-c2cn(C)c(=O)c3cnccc23)cc(OC)c1CN1CC(C(=O)NCCCCCCCCNc2cccc3c2C(=O)N(C2CCC(=O)N(C)C2=O)C3=O)C1] |
| D9 | 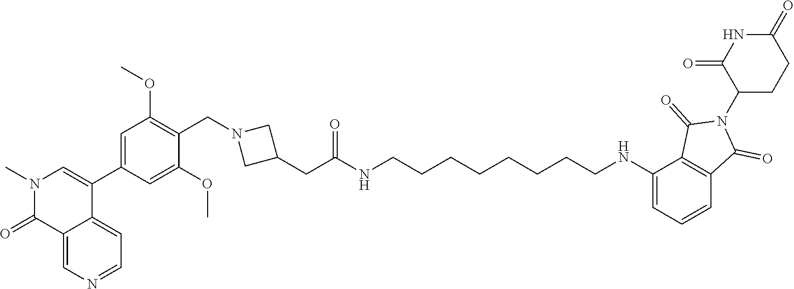 [SMILES: COc1cc(-c2cn(C)c(=O)c3cnccc23)cc(OC)c1CN1CC(CC(=O)NCCCCCCCCNc2cccc3c2C(=O)N(C2CCC(=O)NC2=O)C3=O)C1] |
| D10 | 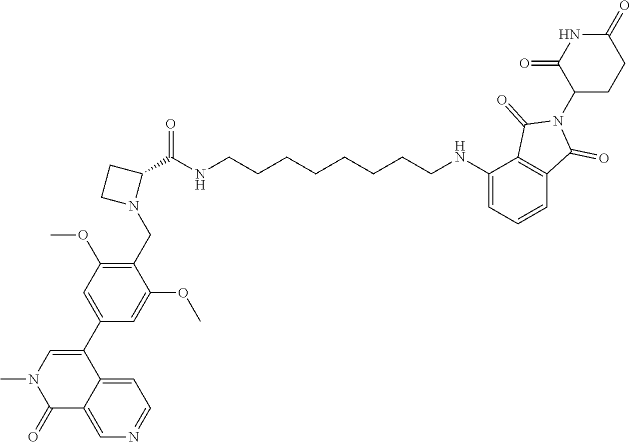 [SMILES: COc1cc(-c2cn(C)c(=O)c3cnccc23)cc(OC)c1CN1CC[C@@H]1C(=O)NCCCCCCCCNc1cccc2c1C(=O)N(C1CCC(=O)NC1=O)C2=O] |
| D11 | 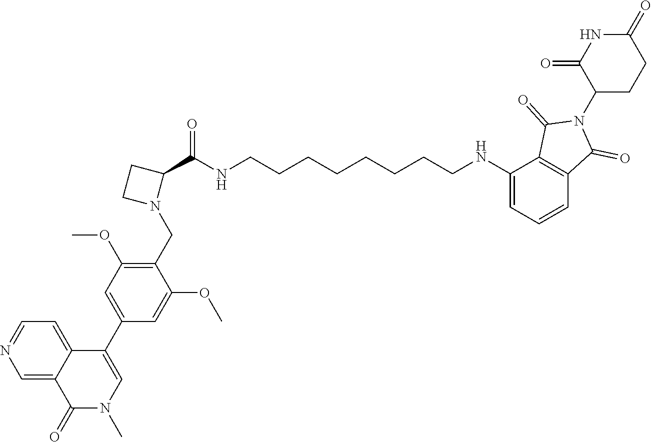 [SMILES: COc1cc(-c2cn(C)c(=O)c3cnccc23)cc(OC)c1CN1CC[C@H]1C(=O)NCCCCCCCCNc1cccc2c1C(=O)N(C1CCC(=O)NC1=O)C2=O] |
| D12 | 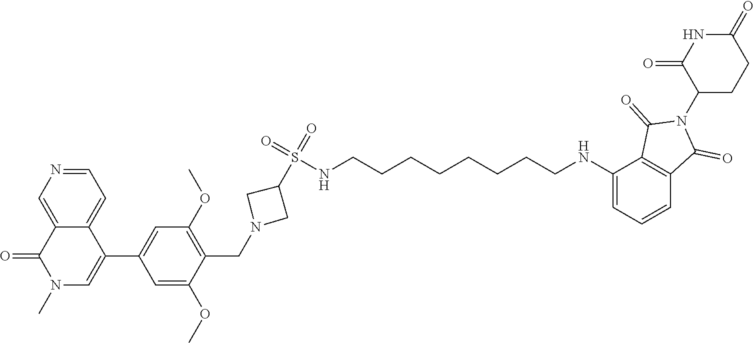 [SMILES: COc1cc(-c2cn(C)c(=O)c3cnccc23)cc(OC)c1CN1CC(S(=O)(=O)NCCCCCCCCNc2cccc3c2C(=O)N(C2CCC(=O)NC2=O)C3=O)C1] |
| D13 | 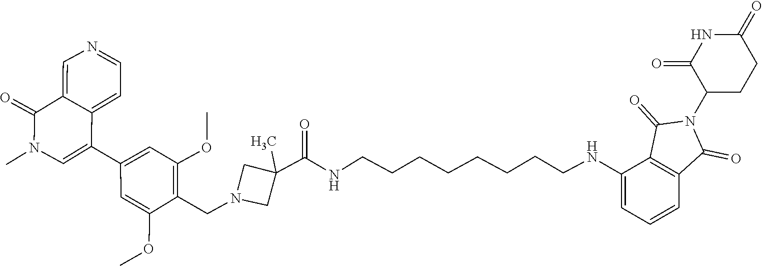 [SMILES: COc1cc(-c2cn(C)c(=O)c3cnccc23)cc(OC)c1CN1CC(C)(C(=O)NCCCCCCCCNc2cccc3c2C(=O)N(C2CCC(=O)NC2=O)C3=O)C1] |
| D14 | 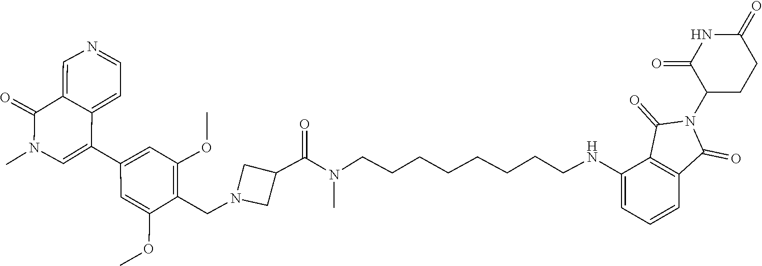 [SMILES: COc1cc(-c2cn(C)c(=O)c3cnccc23)cc(OC)c1CN1CC(C(=O)N(C)CCCCCCCCNc2cccc3c2C(=O)N(C2CCC(=O)NC2=O)C3=O)C1] |
| D15 | 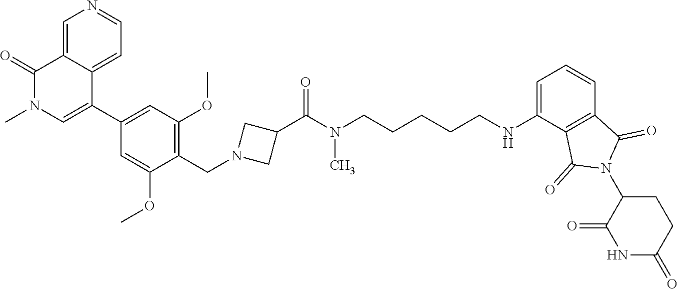 [SMILES: COc1cc(-c2cn(C)c(=O)c3cnccc23)cc(OC)c1CN1CC(C(=O)N(C)CCCCCNc2cccc3c2C(=O)N(C2CCC(=O)NC2=O)C3=O)C1] |
| D16 | 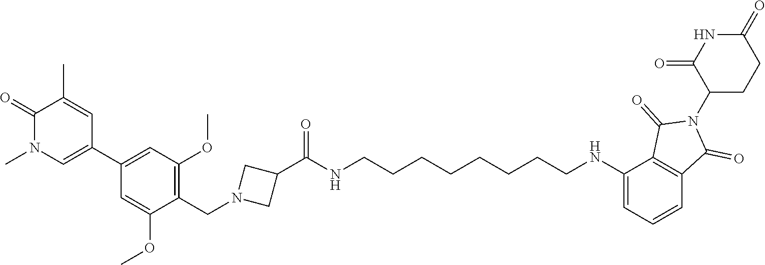 [SMILES: COc1cc(-c2cc(C)c(=O)n(C)c2)cc(OC)c1CN1CC(C(=O)NCCCCCCCCNc2cccc3c2C(=O)N(C2CCC(=O)NC2=O)C3=O)C1] |
| D17 | 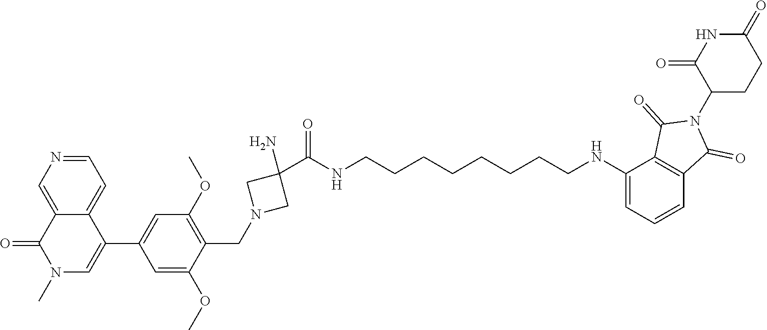 [SMILES: COc1cc(-c2cn(C)c(=O)c3cnccc23)cc(OC)c1CN1CC(N)(C(=O)NCCCCCCCCNc2cccc3c2C(=O)N(C2CCC(=O)NC2=O)C3=O)C1] |
| D18 | 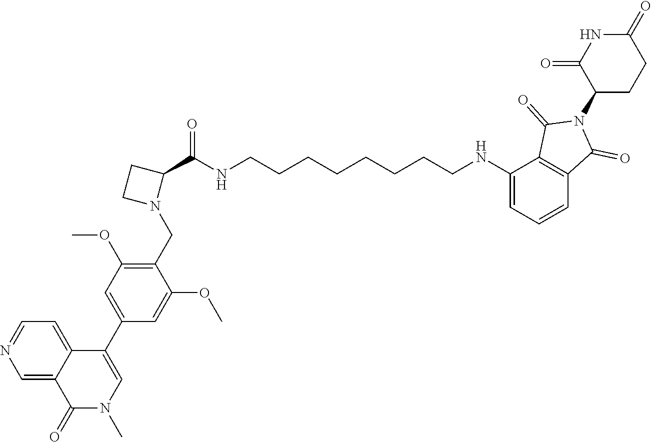 [SMILES: COc1cc(-c2cn(C)c(=O)c3cnccc23)cc(OC)c1CN1CC[C@H]1C(=O)NCCCCCCCCNc1cccc2c1C(=O)N([C@@H]1CCC(=O)NC1=O)C2=O] |
| D19 | 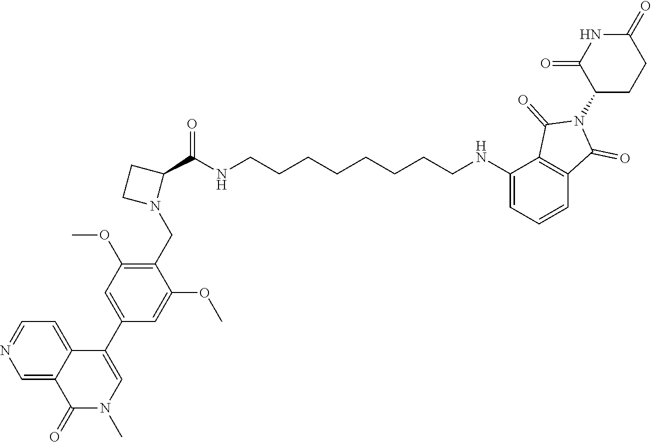 [SMILES: COc1cc(-c2cn(C)c(=O)c3cnccc23)cc(OC)c1CN1CC[C@H]1C(=O)NCCCCCCCCNc1cccc2c1C(=O)N([C@H]1CCC(=O)NC1=O)C2=O] |
| D20 | 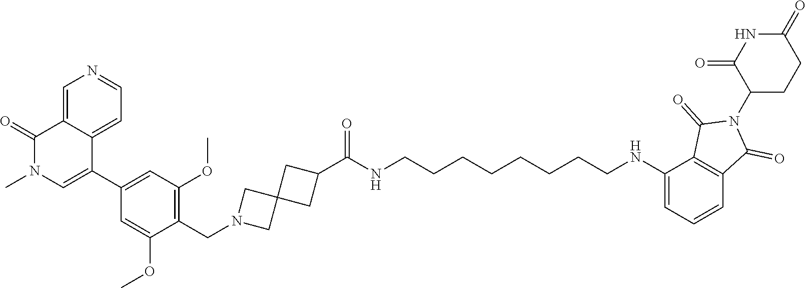 [SMILES: COc1cc(-c2cn(C)c(=O)c3cnccc23)cc(OC)c1CN1CC2(CC(C(=O)NCCCCCCCCNc3cccc4c3C(=O)N(C3CCC(=O)NC3=O)C4=O)C2)C1] |
| D21 | 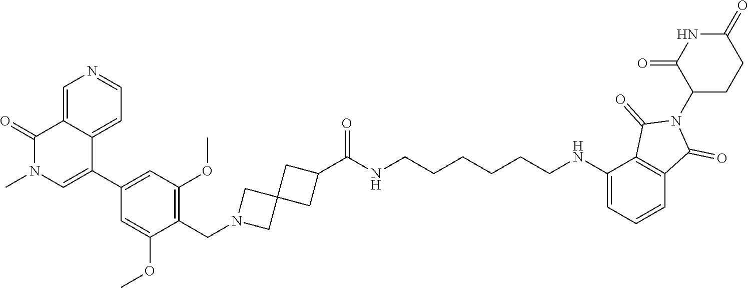 [SMILES: COc1cc(-c2cn(C)c(=O)c3cnccc23)cc(OC)c1CN1CC2(CC(C(=O)NCCCCCCNc3cccc4c3C(=O)N(C3CCC(=O)NC3=O)C4=O)C2)C1] |
| D22 | 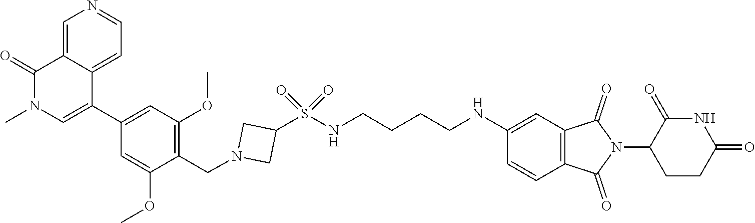 [SMILES: COc1cc(-c2cn(C)c(=O)c3cnccc23)cc(OC)c1CN1CC(S(=O)(=O)NCCCCNc2ccc3c(c2)C(=O)N(C2CCC(=O)NC2=O)C3=O)C1] |
| D23 | 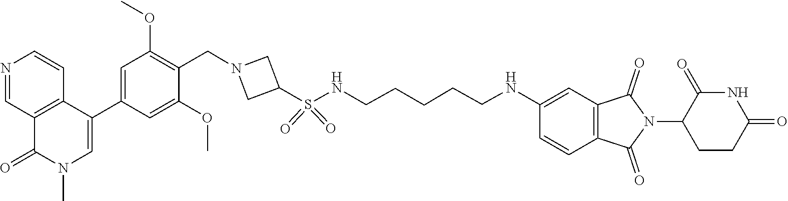 [SMILES: COc1cc(-c2cn(C)c(=O)c3cnccc23)cc(OC)c1CN1CC(S(=O)(=O)NCCCCCNc2ccc3c(c2)C(=O)N(C2CCC(=O)NC2=O)C3=O)C1] |
| D24 | 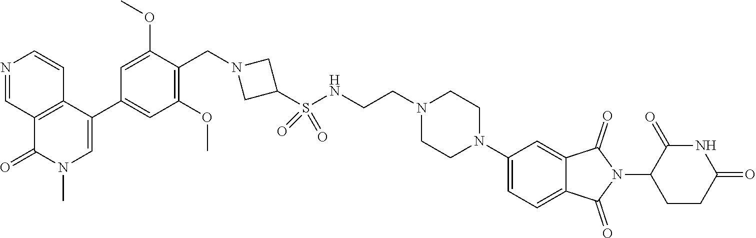 [SMILES: COc1cc(-c2cn(C)c(=O)c3cnccc23)cc(OC)c1CN1CC(S(=O)(=O)NCCN2CCN(c3ccc4c(c3)C(=O)N(C3CCC(=O)NC3=O)C4=O)CC2)C1] |
| D25 | 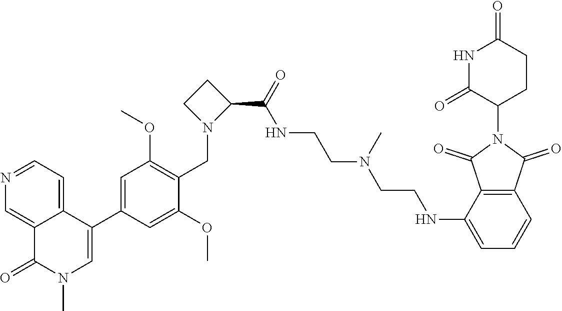 [SMILES: COc1cc(-c2cn(C)c(=O)c3cnccc23)cc(OC)c1CN1CC[C@H]1C(=O)NCCN(C)CCNc1cccc2c1C(=O)N(C1CCC(=O)NC1=O)C2=O] |
| D26 | 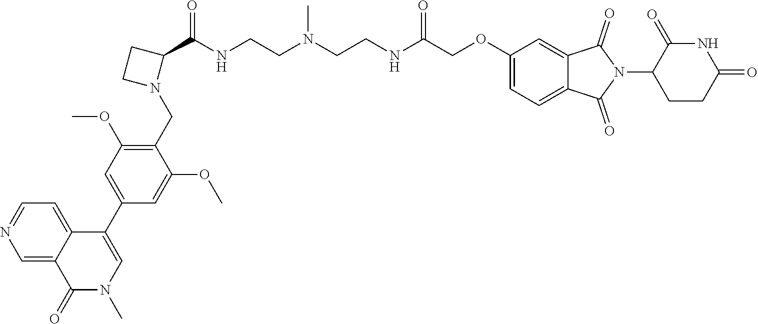 [SMILES: COc1cc(-c2cn(C)c(=O)c3cnccc23)cc(OC)c1CN1CC[C@H]1C(=O)NCCN(C)CCNC(=O)COc1ccc2c(c1)C(=O)N(C1CCC(=O)NC1=O)C2=O] |
| D27 | 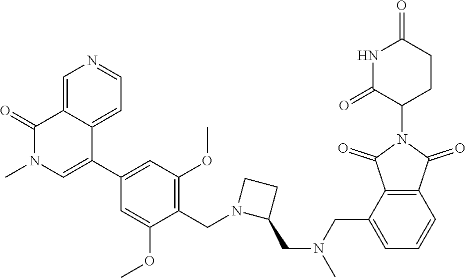 [SMILES: COc1cc(-c2cn(C)c(=O)c3cnccc23)cc(OC)c1CN1CC[C@H]1CN(C)Cc1cccc2c1C(=O)N(C1CCC(=O)NC1=O)C2=O] |
| D28 | 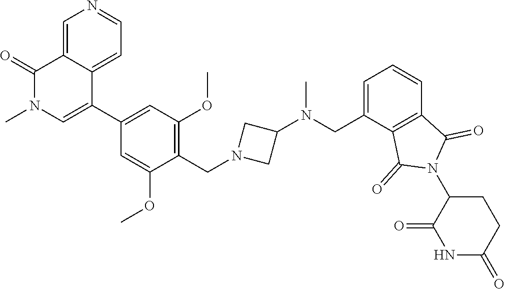 [SMILES: COc1cc(-c2cn(C)c(=O)c3cnccc23)cc(OC)c1CN1CC(N(C)Cc2cccc3c2C(=O)N(C2CCC(=O)NC2=O)C3=O)C1] |
| D29 | 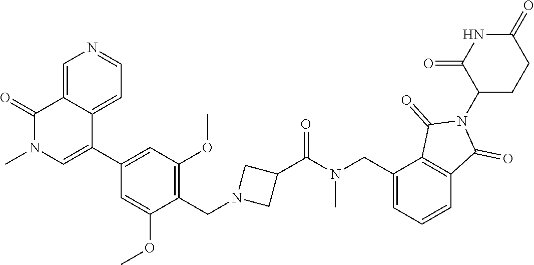 [SMILES: COc1cc(-c2cn(C)c(=O)c3cnccc23)cc(OC)c1CN1CC(C(=O)N(C)Cc2cccc3c2C(=O)N(C2CCC(=O)NC2=O)C3=O)C1] |
| D30 | 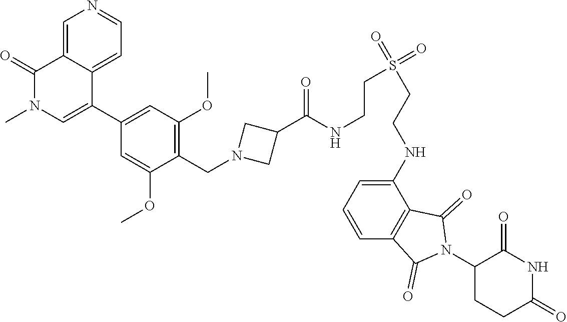 [SMILES: COc1cc(-c2cn(C)c(=O)c3cnccc23)cc(OC)c1CN1CC(C(=O)NCCS(=O)(=O)CCNc2cccc3c2C(=O)N(C2CCC(=O)NC2=O)C3=O)C1] |
| D31 | 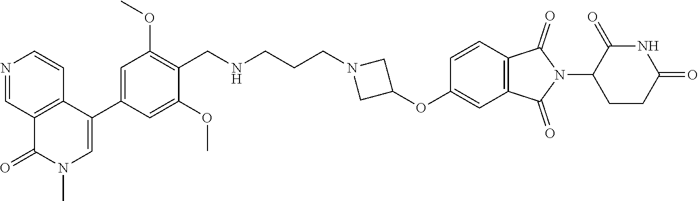 [SMILES: COc1cc(-c2cn(C)c(=O)c3cnccc23)cc(OC)c1CNCCCN1CC(Oc2ccc3c(c2)C(=O)N(C2CCC(=O)NC2=O)C3=O)C1] |
| D32 | 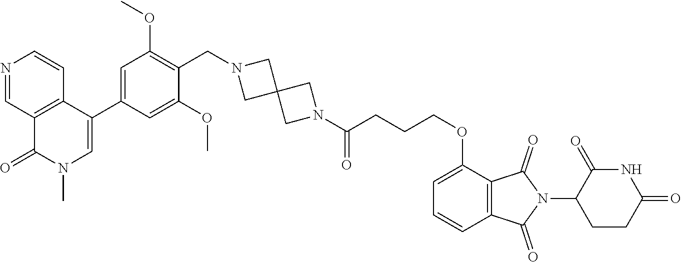 [SMILES: COc1cc(-c2cn(C)c(=O)c3cnccc23)cc(OC)c1CN1CC2(C1)CN(C(=O)CCCOc1cccc3c1C(=O)N(C1CCC(=O)NC1=O)C3=O)C2] |
| D33 | 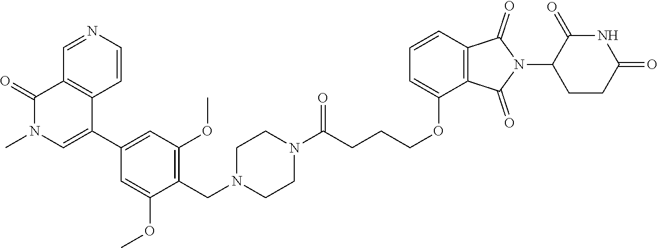 [SMILES: COc1cc(-c2cn(C)c(=O)c3cnccc23)cc(OC)c1CN1CCN(C(=O)CCCOc2cccc3c2C(=O)N(C2CCC(=O)NC2=O)C3=O)CC1] |
| D34 | 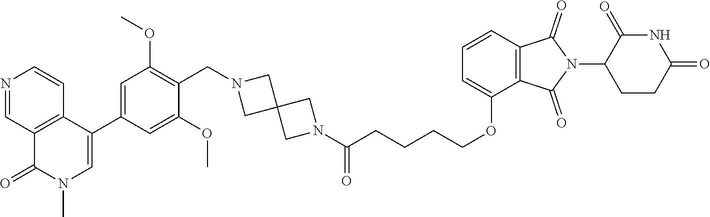 [SMILES: COc1cc(-c2cn(C)c(=O)c3cnccc23)cc(OC)c1CN1CC2(C1)CN(C(=O)CCCCOc1cccc3c1C(=O)N(C1CCC(=O)NC1=O)C3=O)C2] |
| D35 | 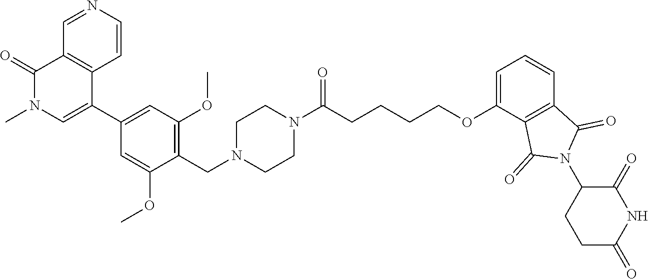 [SMILES: COc1cc(-c2cn(C)c(=O)c3cnccc23)cc(OC)c1CN1CCN(C(=O)CCCCOc2cccc3c2C(=O)N(C2CCC(=O)NC2=O)C3=O)CC1] |
| D36 | 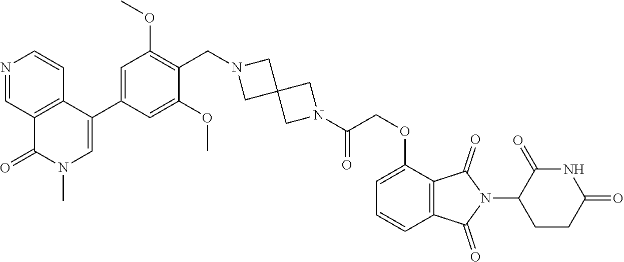 [SMILES: COc1cc(-c2cn(C)c(=O)c3cnccc23)cc(OC)c1CN1CC2(C1)CN(C(=O)COc1cccc3c1C(=O)N(C1CCC(=O)NC1=O)C3=O)C2] |
| D37 | 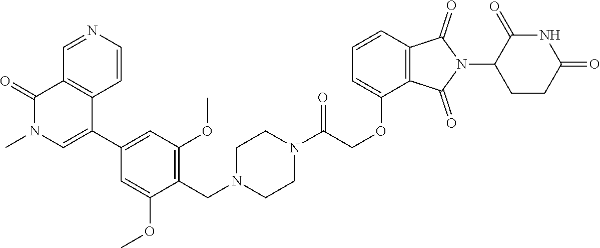 [SMILES: COc1cc(-c2cn(C)c(=O)c3cnccc23)cc(OC)c1CN1CCN(C(=O)COc2cccc3c2C(=O)N(C2CCC(=O)NC2=O)C3=O)CC1] |
| D38 | 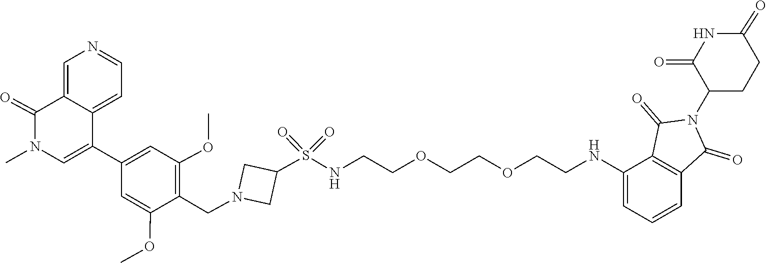 [SMILES: COc1cc(-c2cn(C)c(=O)c3cnccc23)cc(OC)c1CN1CC(S(=O)(=O)NCCOCCOCCNc2cccc3c2C(=O)N(C2CCC(=O)NC2=O)C3=O)C1] |
| D39 | 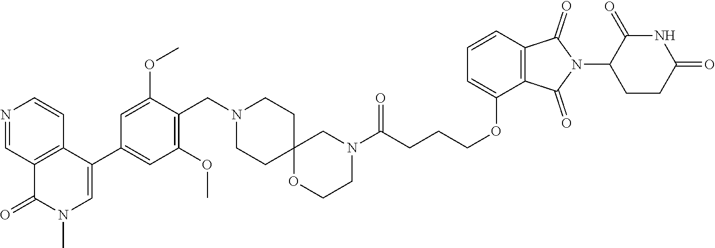 [SMILES: COc1cc(-c2cn(C)c(=O)c3cnccc23)cc(OC)c1CN1CCC2(CC1)CN(C(=O)CCCOc1cccc3c1C(=O)N(C1CCC(=O)NC1=O)C3=O)CCO2] |
| D40 | 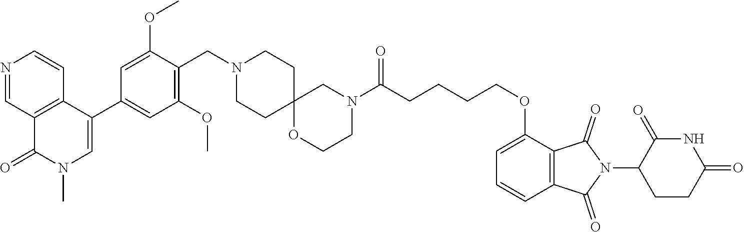 [SMILES: COc1cc(-c2cn(C)c(=O)c3cnccc23)cc(OC)c1CN1CCC2(CC1)CN(C(=O)CCCCOc1cccc3c1C(=O)N(C1CCC(=O)NC1=O)C3=O)CCO2] |
| D41 | 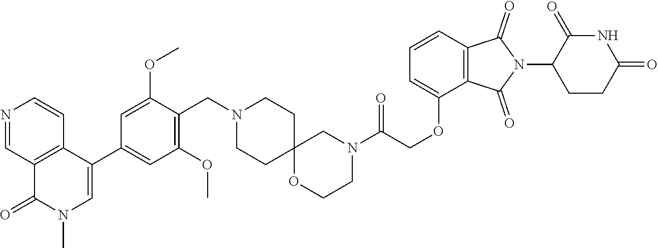 [SMILES: COc1cc(-c2cn(C)c(=O)c3cnccc23)cc(OC)c1CN1CCC2(CC1)CN(C(=O)COc1cccc3c1C(=O)N(C1CCC(=O)NC1=O)C3=O)CCO2] |
| D42 | 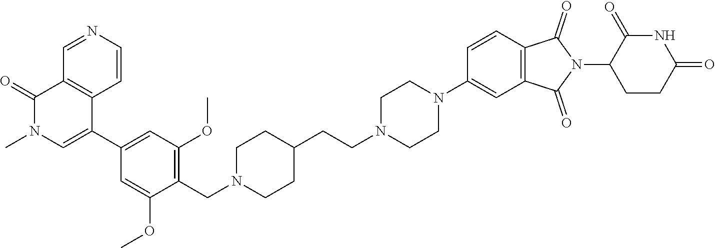 [SMILES: COc1cc(-c2cn(C)c(=O)c3cnccc23)cc(OC)c1CN1CCC(CCN2CCN(c3ccc4c(c3)C(=O)N(C3CCC(=O)NC3=O)C4=O)CC2)CC1] |
| D43 | 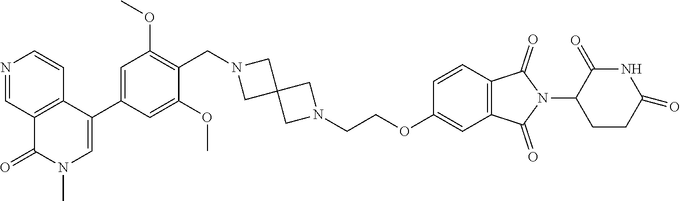 [SMILES: COc1cc(-c2cn(C)c(=O)c3cnccc23)cc(OC)c1CN1CC2(CN(CCOc3ccc4c(c3)C(=O)N(C3CCC(=O)NC3=O)C4=O)C2)C1] |
| D44 | 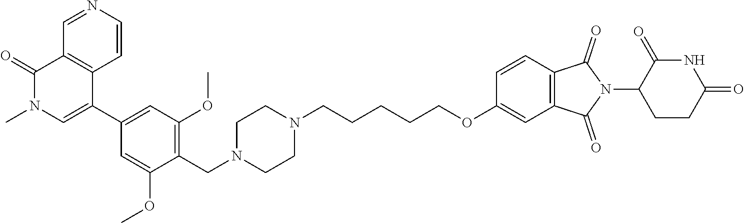 [SMILES: COc1cc(-c2cn(C)c(=O)c3cnccc23)cc(OC)c1CN1CCN(CCCCCOc2ccc3c(c2)C(=O)N(C2CCC(=O)NC2=O)C3=O)CC1] |
| D45 | 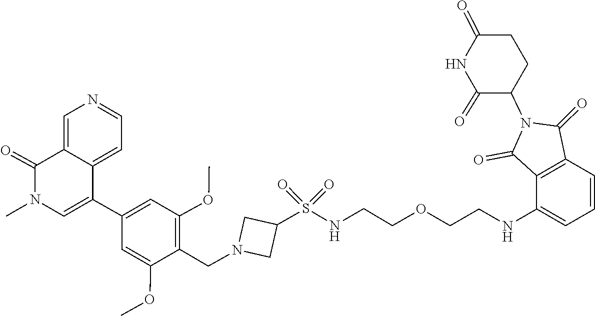 [SMILES: COc1cc(-c2cn(C)c(=O)c3cnccc23)cc(OC)c1CN1CC(S(=O)(=O)NCCOCCNc2cccc3c2C(=O)N(C2CCC(=O)NC2=O)C3=O)C1] |
| D46 | 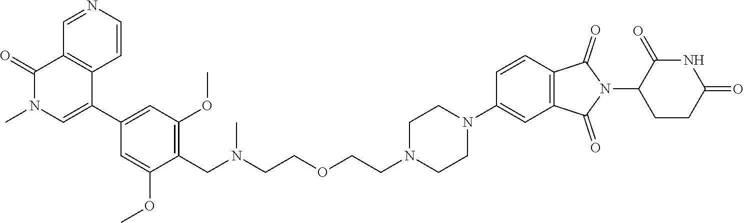 [SMILES: COc1cc(-c2cn(C)c(=O)c3cnccc23)cc(OC)c1CN(C)CCOCCN1CCN(c2ccc3c(c2)C(=O)N(C2CCC(=O)NC2=O)C3=O)CC1] |
| D47 | 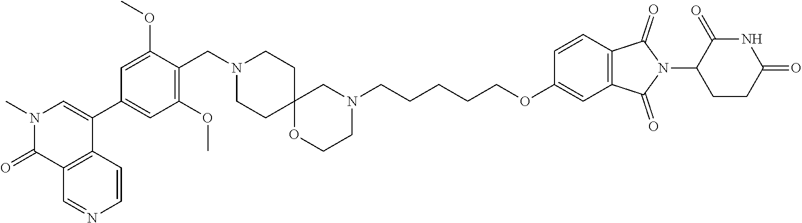 [SMILES: COc1cc(-c2cn(C)c(=O)c3cnccc23)cc(OC)c1CN1CCC2(CC1)CN(CCCCCOc1ccc3c(c1)C(=O)N(C1CCC(=O)NC1=O)C3=O)CCO2] |
| D48 | 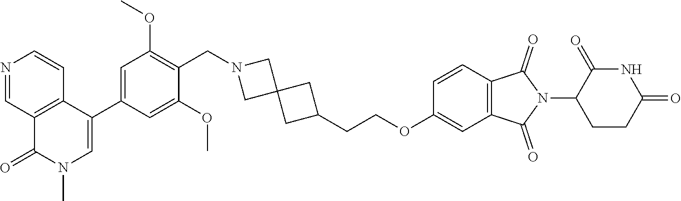 [SMILES: COc1cc(-c2cn(C)c(=O)c3cnccc23)cc(OC)c1CN1CC2(CC(CCOc3ccc4c(c3)C(=O)N(C3CCC(=O)NC3=O)C4=O)C2)C1] |
| D49 | 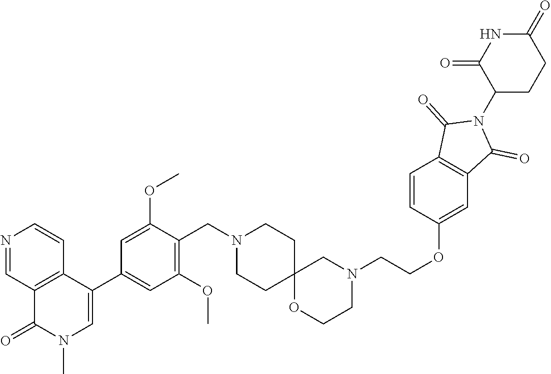 [SMILES: COc1cc(-c2cn(C)c(=O)c3cnccc23)cc(OC)c1CN1CCC2(CC1)CN(CCOc1ccc3c(c1)C(=O)N(C1CCC(=O)NC1=O)C3=O)CCO2] |
| D50 | 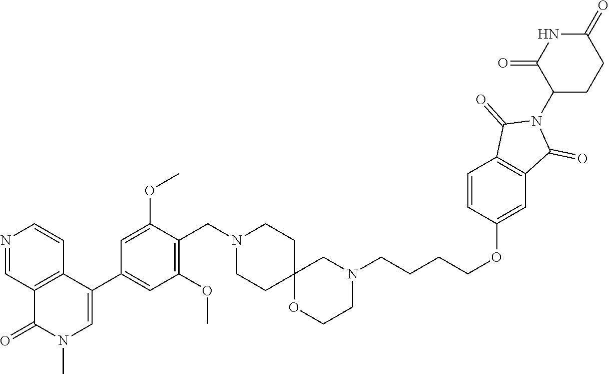 [SMILES: COc1cc(-c2cn(C)c(=O)c3cnccc23)cc(OC)c1CN1CCC2(CC1)CN(CCCCOc1ccc3c(c1)C(=O)N(C1CCC(=O)NC1=O)C3=O)CCO2] |
| D51 | 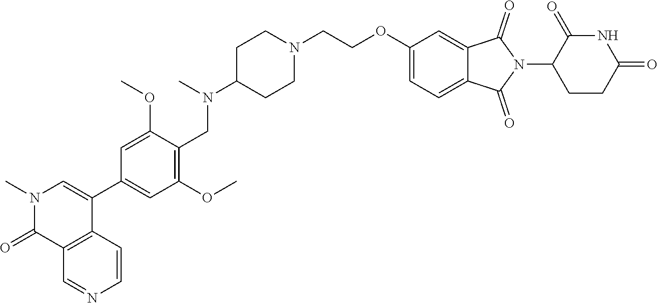 [SMILES: COc1cc(-c2cn(C)c(=O)c3cnccc23)cc(OC)c1CN(C)C1CCN(CCOc2ccc3c(c2)C(=O)N(C2CCC(=O)NC2=O)C3=O)CC1] |
| D52 | 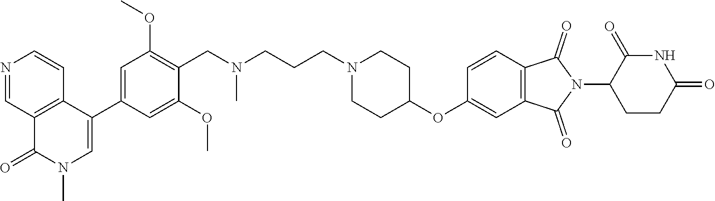 [SMILES: COc1cc(-c2cn(C)c(=O)c3cnccc23)cc(OC)c1CN(C)CCCN1CCC(Oc2ccc3c(c2)C(=O)N(C2CCC(=O)NC2=O)C3=O)CC1] |
| D53 | 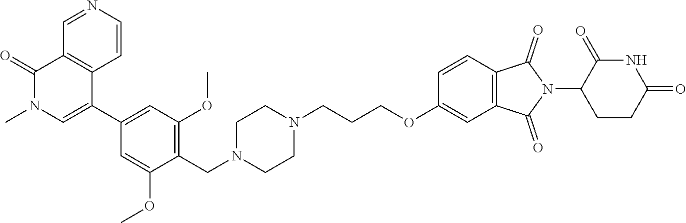 [SMILES: COc1cc(-c2cn(C)c(=O)c3cnccc23)cc(OC)c1CN1CCN(CCCOc2ccc3c(c2)C(=O)N(C2CCC(=O)NC2=O)C3=O)CC1] |
| D54 | 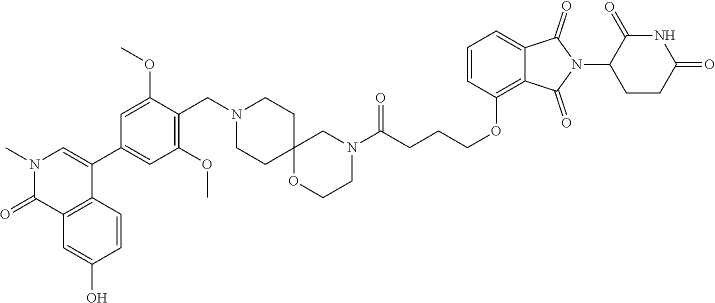 [SMILES: COc1cc(-c2cn(C)c(=O)c3cc(O)ccc23)cc(OC)c1CN1CCC2(CC1)CN(C(=O)CCCOc1cccc3c1C(=O)N(C1CCC(=O)NC1=O)C3=O)CCO2] |
| D55 | 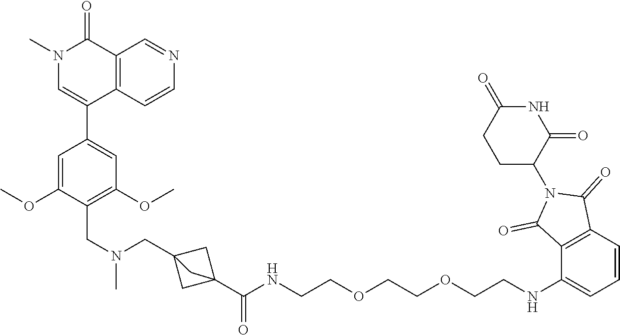 [SMILES: COc1cc(-c2cn(C)c(=O)c3cnccc23)cc(OC)c1CN(C)CC12CC(C(=O)NCCOCCOCCNc3cccc4c3C(=O)N(C3CCC(=O)NC3=O)C4=O)(C1)C2] |
| D56 | 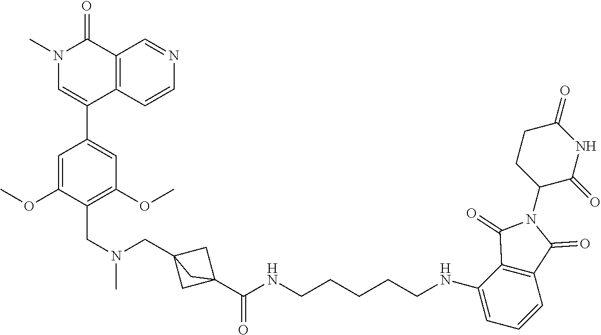 [SMILES: COc1cc(-c2cn(C)c(=O)c3cnccc23)cc(OC)c1CN(C)CC12CC(C(=O)NCCCCCNc3cccc4c3C(=O)N(C3CCC(=O)NC3=O)C4=O)(C1)C2] |
| D57 | 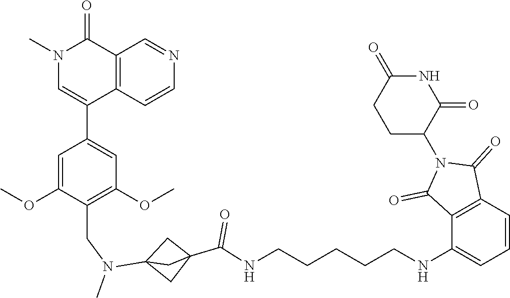 [SMILES: COc1cc(-c2cn(C)c(=O)c3cnccc23)cc(OC)c1CN(C)C12CC(C(=O)NCCCCCNc3cccc4c3C(=O)N(C3CCC(=O)NC3=O)C4=O)(C1)C2] |
| D58 | 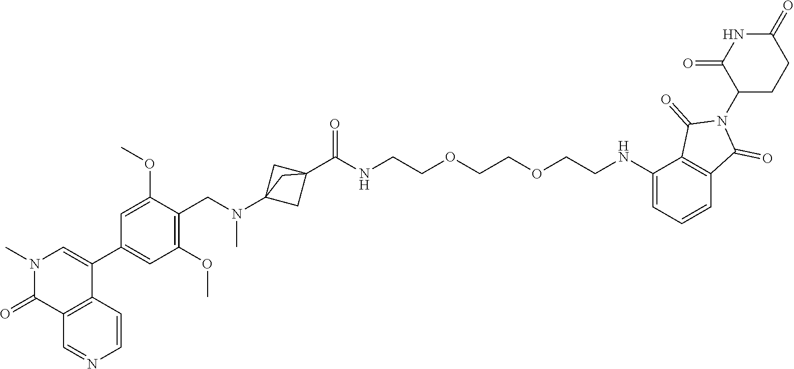 [SMILES: COc1cc(-c2cn(C)c(=O)c3cnccc23)cc(OC)c1CN(C)C12CC(C(=O)NCCOCCOCCNc3cccc4c3C(=O)N(C3CCC(=O)NC3=O)C4=O)(C1)C2] |
| D59 | 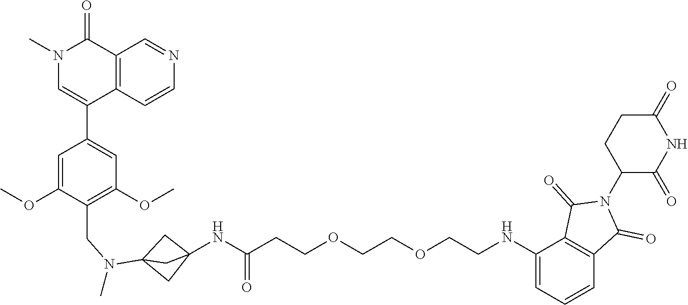 [SMILES: COc1cc(-c2cn(C)c(=O)c3cnccc23)cc(OC)c1CN(C)C12CC(NC(=O)CCOCCOCCNc3cccc4c3C(=O)N(C3CCC(=O)NC3=O)C4=O)(C1)C2] |
| D60 | 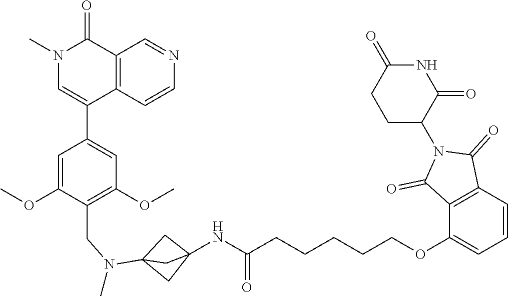 [SMILES: COc1cc(-c2cn(C)c(=O)c3cnccc23)cc(OC)c1CN(C)C12CC(NC(=O)CCCCCOc3cccc4c3C(=O)N(C3CCC(=O)NC3=O)C4=O)(C1)C2] |
| D61 | 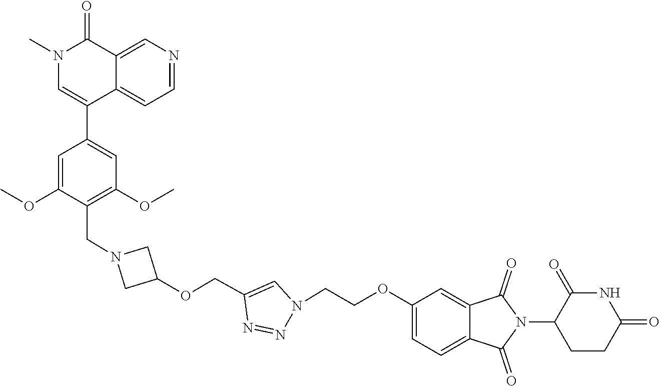 [SMILES: COc1cc(-c2cn(C)c(=O)c3cnccc23)cc(OC)c1CN1CC(OCc2cn(CCOc3ccc4c(c3)C(=O)N(C3CCC(=O)NC3=O)C4=O)nn2)C1] |
| D62 | 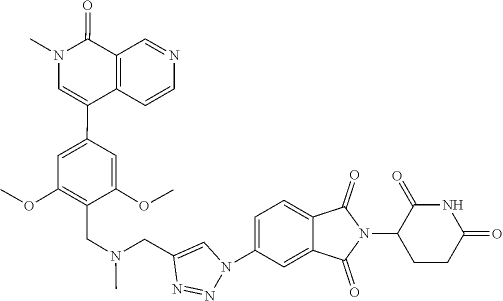 [SMILES: COc1cc(-c2cn(C)c(=O)c3cnccc23)cc(OC)c1CN(C)Cc1cn(-c2ccc3c(c2)C(=O)N(C2CCC(=O)NC2=O)C3=O)nn1] |
| D63 | 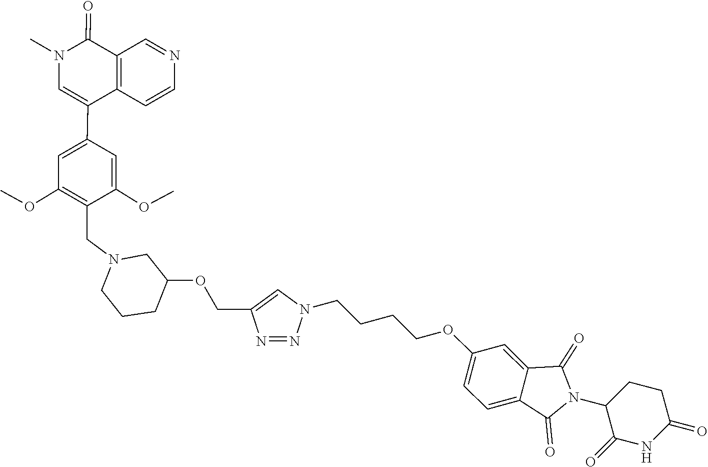 [SMILES: COc1cc(-c2cn(C)c(=O)c3cnccc23)cc(OC)c1CN1CCCC(OCc2cn(CCCCOc3ccc4c(c3)C(=O)N(C3CCC(=O)NC3=O)C4=O)nn2)C1] |
| D64 | 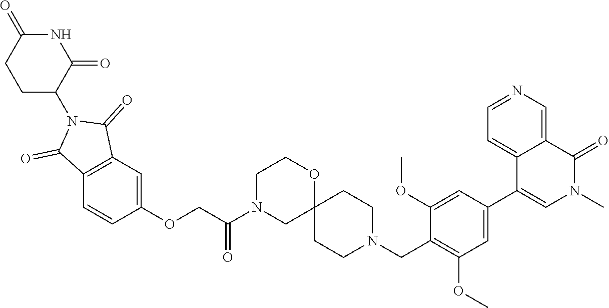 [SMILES: COc1cc(-c2cn(C)c(=O)c3cnccc23)cc(OC)c1CN1CCC2(CC1)CN(C(=O)COc1ccc3c(c1)C(=O)N(C1CCC(=O)NC1=O)C3=O)CCO2] |
| D65 | 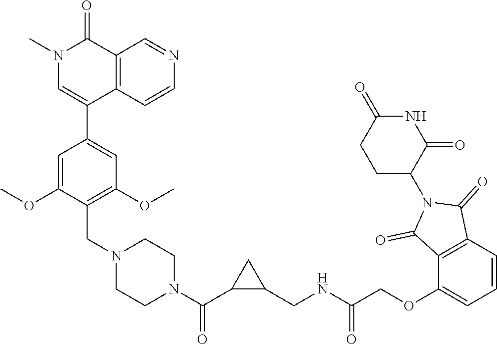 [SMILES: COc1cc(-c2cn(C)c(=O)c3cnccc23)cc(OC)c1CN1CCN(C(=O)C2CC2CNC(=O)COc2cccc3c2C(=O)N(C2CCC(=O)NC2=O)C3=O)CC1] |
| D66 | 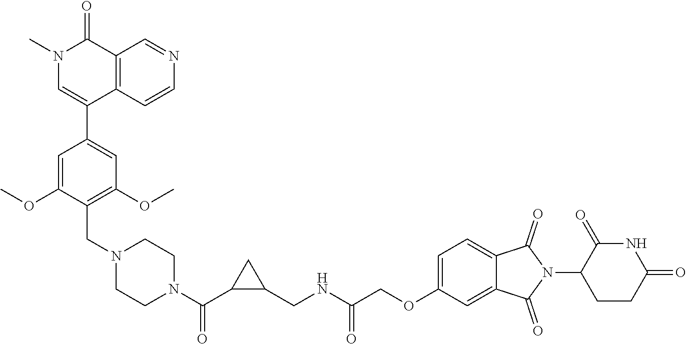 [SMILES: COc1cc(-c2cn(C)c(=O)c3cnccc23)cc(OC)c1CN1CCN(C(=O)C2CC2CNC(=O)COc2ccc3c(c2)C(=O)N(C2CCC(=O)NC2=O)C3=O)CC1] |
| D67 | 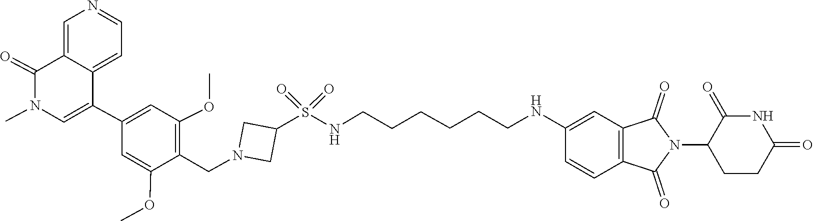 [SMILES: COc1cc(-c2cn(C)c(=O)c3cnccc23)cc(OC)c1CN1CC(S(=O)(=O)NCCCCCCNc2ccc3c(c2)C(=O)N(C2CCC(=O)NC2=O)C3=O)C1] |
| D68 | 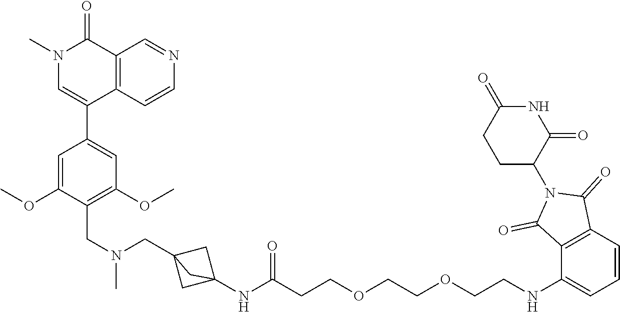 [SMILES: COc1cc(-c2cn(C)c(=O)c3cnccc23)cc(OC)c1CN(C)CC12CC(NC(=O)CCOCCOCCNc3cccc4c3C(=O)N(C3CCC(=O)NC3=O)C4=O)(C1)C2] |
| D69 | 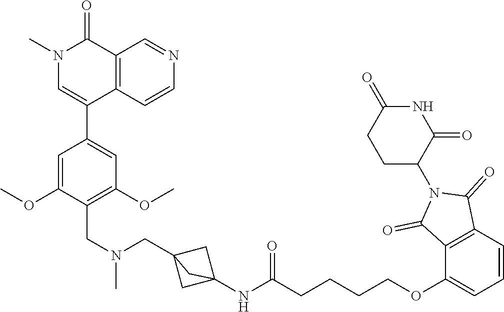 [SMILES: COc1cc(-c2cn(C)c(=O)c3cnccc23)cc(OC)c1CN(C)CC12CC(NC(=O)CCCCOc3cccc4c3C(=O)N(C3CCC(=O)NC3=O)C4=O)(C1)C2] |
| D70 | 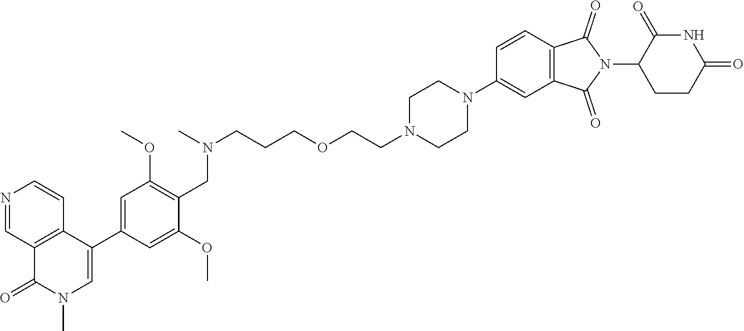 [SMILES: COc1cc(-c2cn(C)c(=O)c3cnccc23)cc(OC)c1CN(C)CCCOCCN1CCN(c2ccc3c(c2)C(=O)N(C2CCC(=O)NC2=O)C3=O)CC1] |
| D71 | 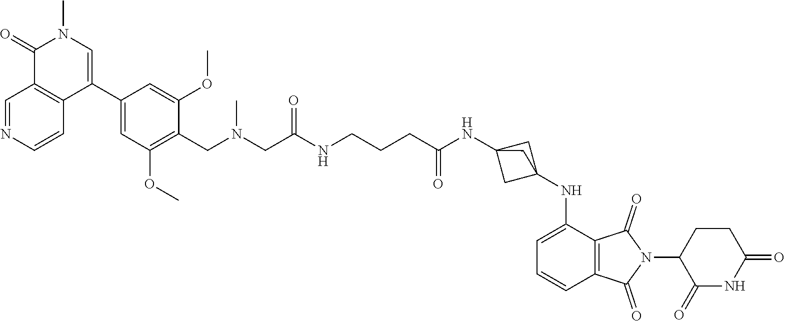 [SMILES: COc1cc(-c2cn(C)c(=O)c3cnccc23)cc(OC)c1CN(C)CC(=O)NCCCC(=O)NC12CC(Nc3cccc4c3C(=O)N(C3CCC(=O)NC3=O)C4=O)(C1)C2] |
| D72 | 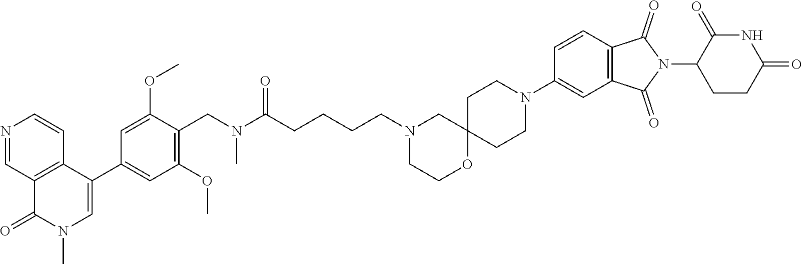 [SMILES: COc1cc(-c2cn(C)c(=O)c3cnccc23)cc(OC)c1CN(C)C(=O)CCCCN1CCOC2(CCN(c3ccc4c(c3)C(=O)N(C3CCC(=O)NC3=O)C4=O)CC2)C1] |
| D73 | 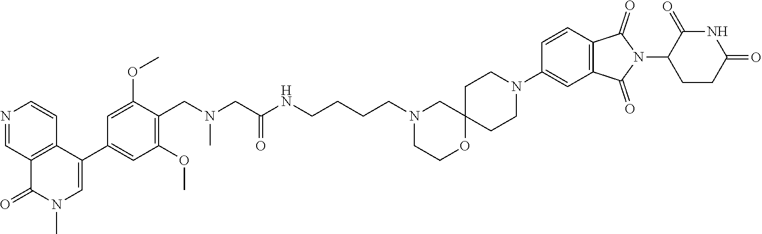 [SMILES: COc1cc(-c2cn(C)c(=O)c3cnccc23)cc(OC)c1CN(C)CC(=O)NCCCCN1CCOC2(CCN(c3ccc4c(c3)C(=O)N(C3CCC(=O)NC3=O)C4=O)CC2)C1] |
| D74 | 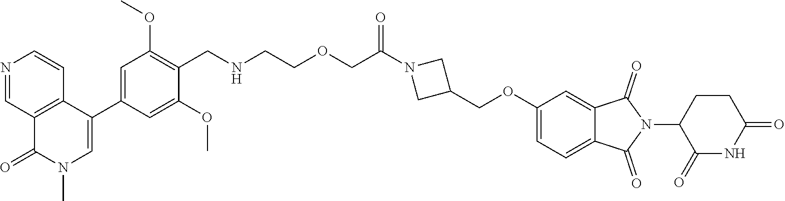 [SMILES: COc1cc(-c2cn(C)c(=O)c3cnccc23)cc(OC)c1CNCCOCC(=O)N1CC(COc2ccc3c(c2)C(=O)N(C2CCC(=O)NC2=O)C3=O)C1] |
| D75 | 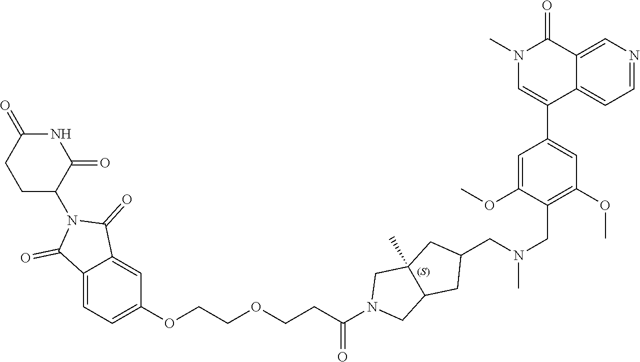 |
| D76 | 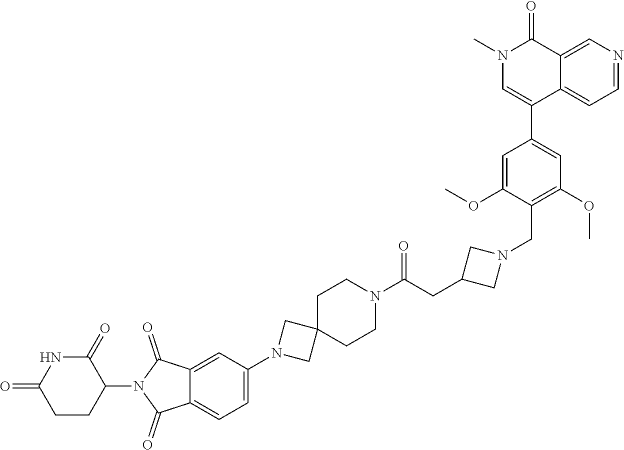 [SMILES: COc1cc(-c2cn(C)c(=O)c3cnccc23)cc(OC)c1CN1CC(CC(=O)N2CCC3(CC2)CN(c2ccc4c(c2)C(=O)N(C2CCC(=O)NC2=O)C4=O)C3)C1] |
| D77 | 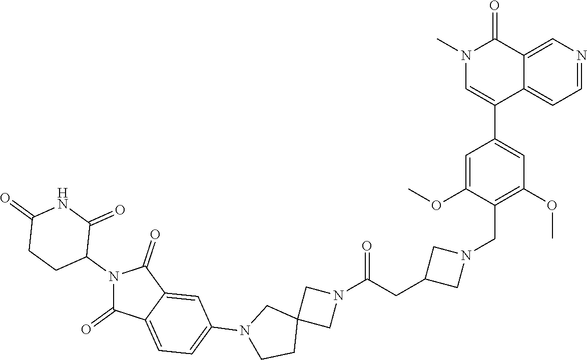 [SMILES: COc1cc(-c2cn(C)c(=O)c3cnccc23)cc(OC)c1CN1CC(CC(=O)N2CC3(CCN(c4ccc5c(c4)C(=O)N(C4CCC(=O)NC4=O)C5=O)C3)C2)C1] |
| D78 | 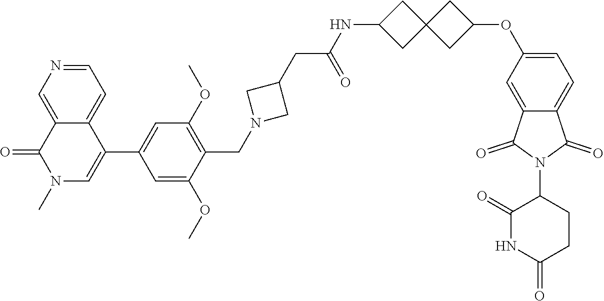 [SMILES: COc1cc(-c2cn(C)c(=O)c3cnccc23)cc(OC)c1CN1CC(CC(=O)NC2CC3(C2)CC(Oc2ccc4c(c2)C(=O)N(C2CCC(=O)NC2=O)C4=O)C3)C1] |
| D79 | 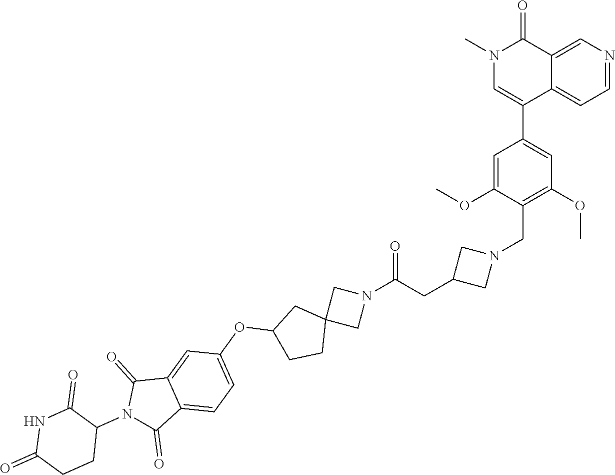 [SMILES: COc1cc(-c2cn(C)c(=O)c3cnccc23)cc(OC)c1CN1CC(CC(=O)N2CC3(CCC(Oc4ccc5c(c4)C(=O)N(C4CCC(=O)NC4=O)C5=O)C3)C2)C1] |
| D80 | 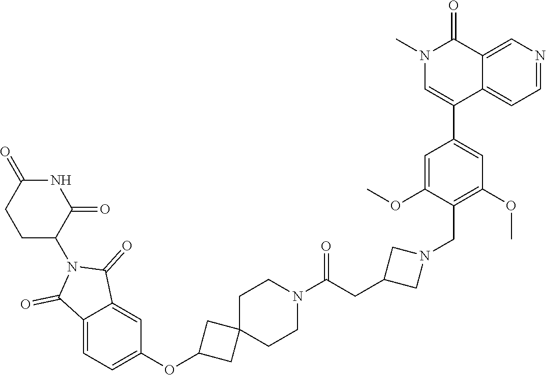 [SMILES: COc1cc(-c2cn(C)c(=O)c3cnccc23)cc(OC)c1CN1CC(CC(=O)N2CCC3(CC2)CC(Oc2ccc4c(c2)C(=O)N(C2CCC(=O)NC2=O)C4=O)C3)C1] |
| D81 | 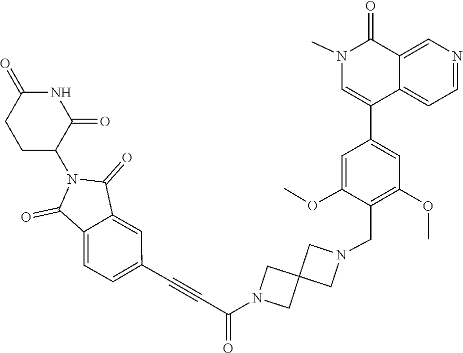 [SMILES: COc1cc(-c2cn(C)c(=O)c3cnccc23)cc(OC)c1CN1CC2(C1)CN(C(=O)C#Cc1ccc3c(c1)C(=O)N(C1CCC(=O)NC1=O)C3=O)C2] |
| D82 | 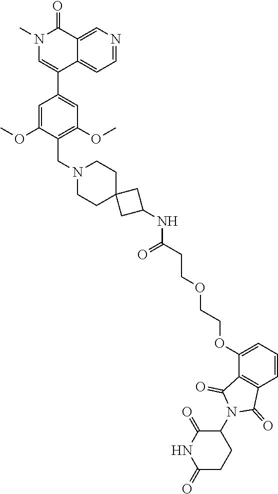 [SMILES: COc1cc(-c2cn(C)c(=O)c3cnccc23)cc(OC)c1CN1CCC2(CC1)CC(NC(=O)CCOCCOc1cccc3c1C(=O)N(C1CCC(=O)NC1=O)C3=O)C2] |
| D83 | 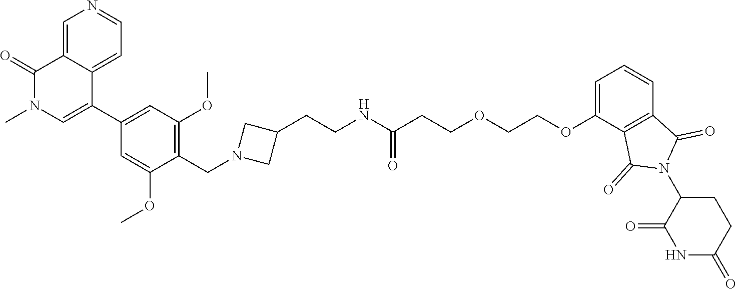 [SMILES: COc1cc(-c2cn(C)c(=O)c3cnccc23)cc(OC)c1CN1CC(CCNC(=O)CCOCCOc2cccc3c2C(=O)N(C2CCC(=O)NC2=O)C3=O)C1] |
| D84 | 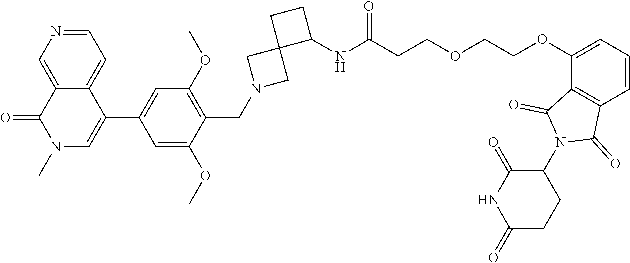 [SMILES: COc1cc(-c2cn(C)c(=O)c3cnccc23)cc(OC)c1CN1CC2(CCC2NC(=O)CCOCCOc2cccc3c2C(=O)N(C2CCC(=O)NC2=O)C3=O)C1] |
| D85 | 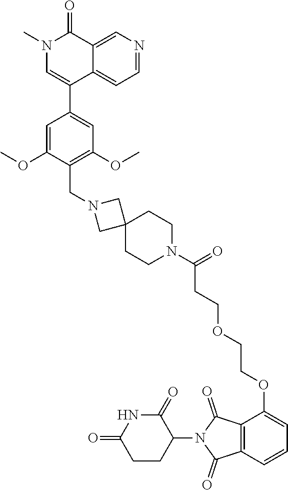 [SMILES: COc1cc(-c2cn(C)c(=O)c3cnccc23)cc(OC)c1CN1CC2(CCN(C(=O)CCOCCOc3cccc4c3C(=O)N(C3CCC(=O)NC3=O)C4=O)CC2)C1] |
| D86 | 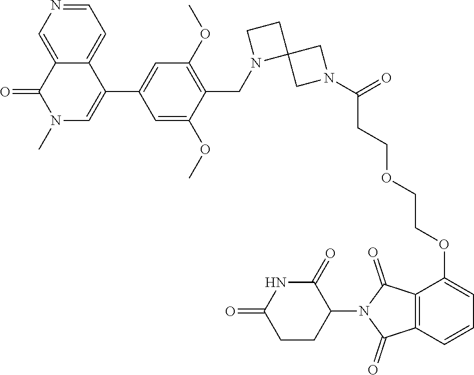 [SMILES: COc1cc(-c2cn(C)c(=O)c3cnccc23)cc(OC)c1CN1CCC12CN(C(=O)CCOCCOc1cccc3c1C(=O)N(C1CCC(=O)NC1=O)C3=O)C2] |
| D87 | 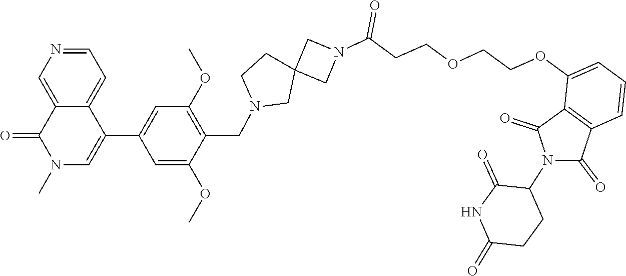 [SMILES: COc1cc(-c2cn(C)c(=O)c3cnccc23)cc(OC)c1CN1CCC2(C1)CN(C(=O)CCOCCOc1cccc3c1C(=O)N(C1CCC(=O)NC1=O)C3=O)C2] |
| D88 | 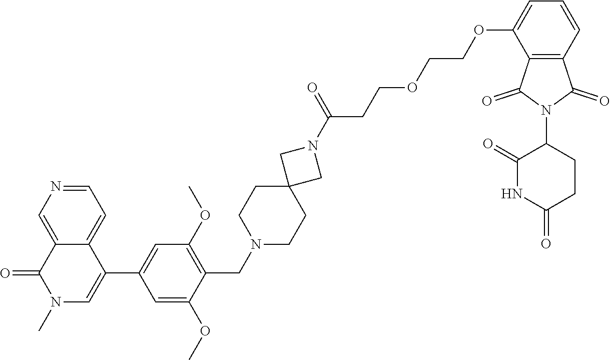 [SMILES: COc1cc(-c2cn(C)c(=O)c3cnccc23)cc(OC)c1CN1CCC2(CC1)CN(C(=O)CCOCCOc1cccc3c1C(=O)N(C1CCC(=O)NC1=O)C3=O)C2] |
| D89 | 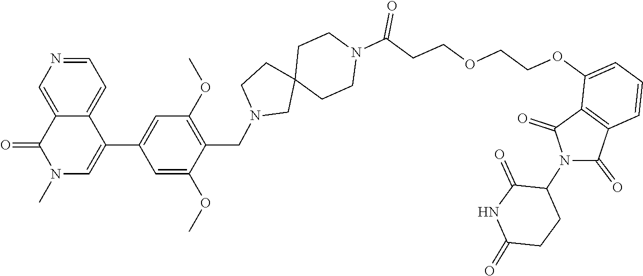 [SMILES: COc1cc(-c2cn(C)c(=O)c3cnccc23)cc(OC)c1CN1CCC2(CCN(C(=O)CCOCCOc3cccc4c3C(=O)N(C3CCC(=O)NC3=O)C4=O)CC2)C1] |
| D90 | 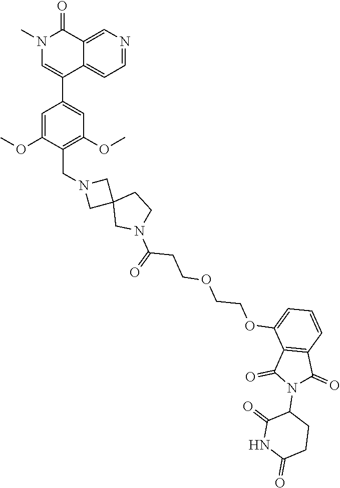 [SMILES: COc1cc(-c2cn(C)c(=O)c3cnccc23)cc(OC)c1CN1CC2(CCN(C(=O)CCOCCOc3cccc4c3C(=O)N(C3CCC(=O)NC3=O)C4=O)C2)C1] |
| D91 | 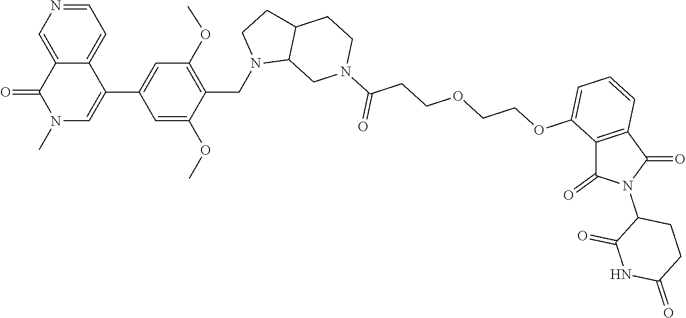 [SMILES: COc1cc(-c2cn(C)c(=O)c3cnccc23)cc(OC)c1CN1CCC2CCN(C(=O)CCOCCOc3cccc4c3C(=O)N(C3CCC(=O)NC3=O)C4=O)CC21] |
| D92 | 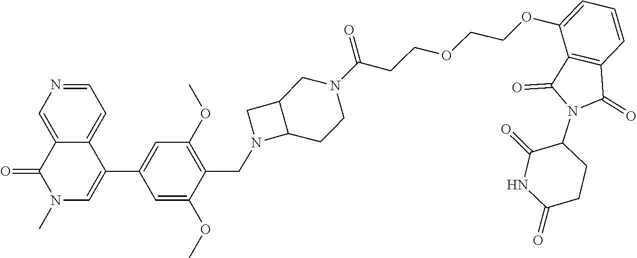 [SMILES: COc1cc(-c2cn(C)c(=O)c3cnccc23)cc(OC)c1CN1CC2CN(C(=O)CCOCCOc3cccc4c3C(=O)N(C3CCC(=O)NC3=O)C4=O)CCC21] |
| D93 | 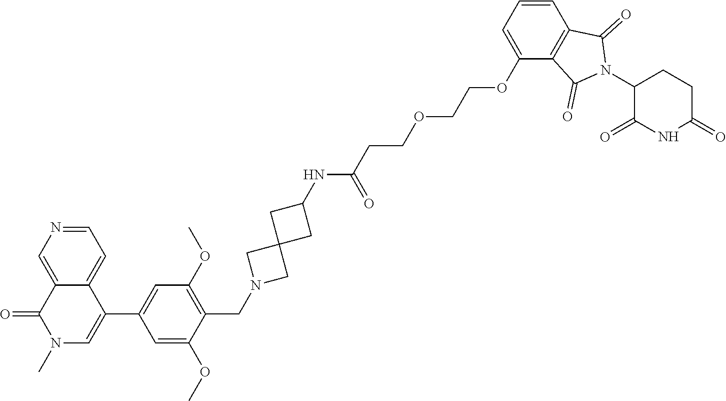 [SMILES: COc1cc(-c2cn(C)c(=O)c3cnccc23)cc(OC)c1CN1CC2(CC(NC(=O)CCOCCOc3cccc4c3C(=O)N(C3CCC(=O)NC3=O)C4=O)C2)C1] |
| D94 | 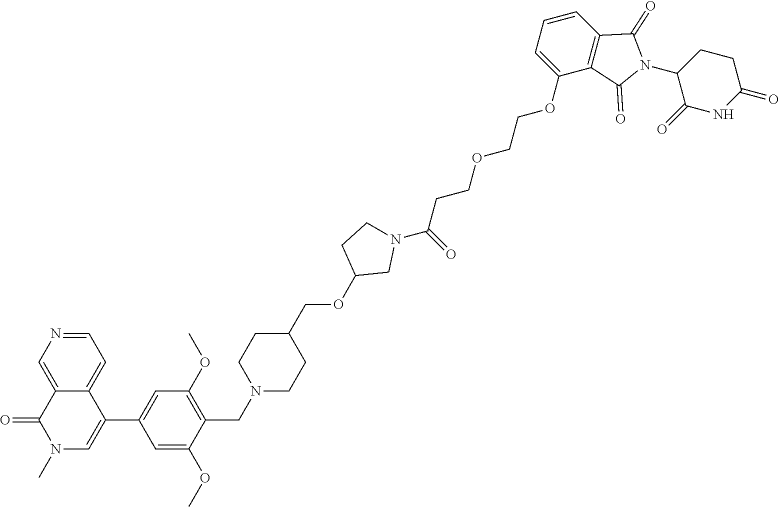 [SMILES: COc1cc(-c2cn(C)c(=O)c3cnccc23)cc(OC)c1CN1CCC(COC2CCN(C(=O)CCOCCOc3cccc4c3C(=O)N(C3CCC(=O)NC3=O)C4=O)C2)CC1] |
| D95 | 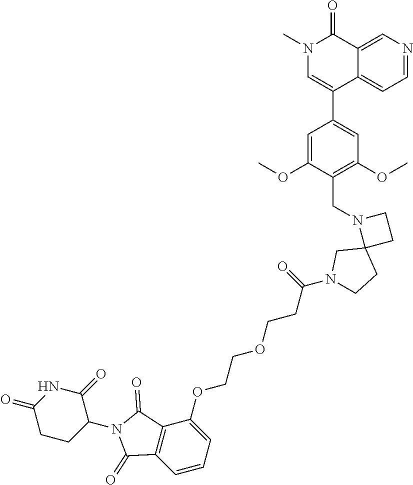 [SMILES: COc1cc(-c2cn(C)c(=O)c3cnccc23)cc(OC)c1CN1CCC12CCN(C(=O)CCOCCOc1cccc3c1C(=O)N(C1CCC(=O)NC1=O)C3=O)C2] |
| D96 | 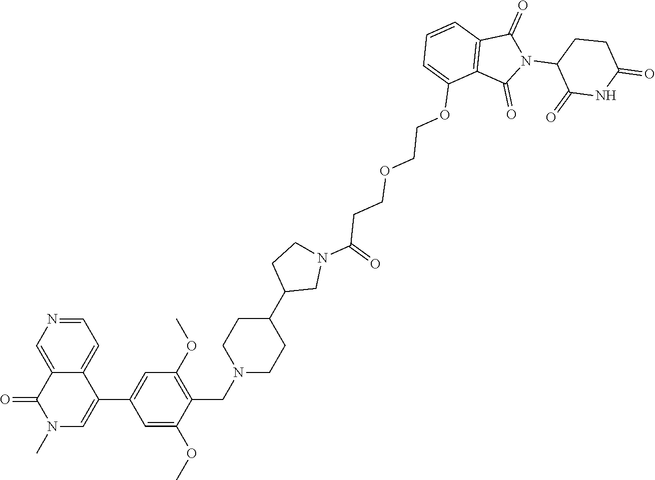 [SMILES: COc1cc(-c2cn(C)c(=O)c3cnccc23)cc(OC)c1CN1CCC(C2CCN(C(=O)CCOCCOc3cccc4c3C(=O)N(C3CCC(=O)NC3=O)C4=O)C2)CC1] |
| D97 | 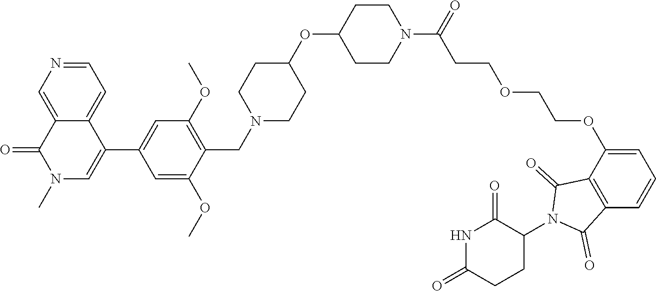 [SMILES: COc1cc(-c2cn(C)c(=O)c3cnccc23)cc(OC)c1CN1CCC(OC2CCN(C(=O)CCOCCOc3cccc4c3C(=O)N(C3CCC(=O)NC3=O)C4=O)CC2)CC1] |
| D98 | 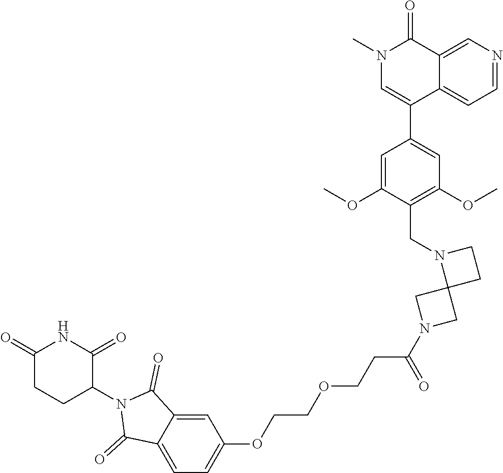 [SMILES: COc1cc(-c2cn(C)c(=O)c3cnccc23)cc(OC)c1CN1CCC12CN(C(=O)CCOCCOc1ccc3c(c1)C(=O)N(C1CCC(=O)NC1=O)C3=O)C2] |
| D99 | 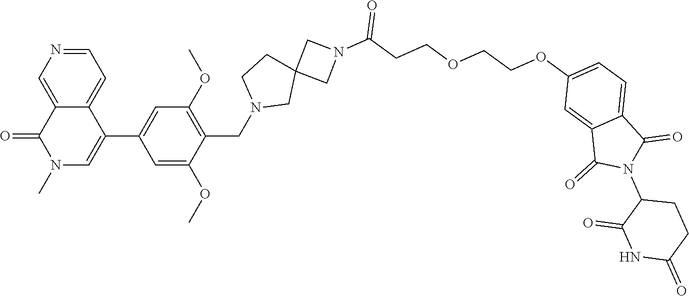 [SMILES: COc1cc(-c2cn(C)c(=O)c3cnccc23)cc(OC)c1CN1CCC2(C1)CN(C(=O)CCOCCOc1ccc3c(c1)C(=O)N(C1CCC(=O)NC1=O)C3=O)C2] |
| D100 | 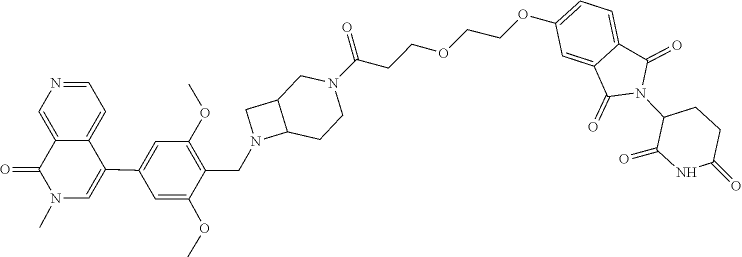 [SMILES: COc1cc(-c2cn(C)c(=O)c3cnccc23)cc(OC)c1CN1CC2CN(C(=O)CCOCCOc3ccc4c(c3)C(=O)N(C3CCC(=O)NC3=O)C4=O)CCC21] |
| D101 | 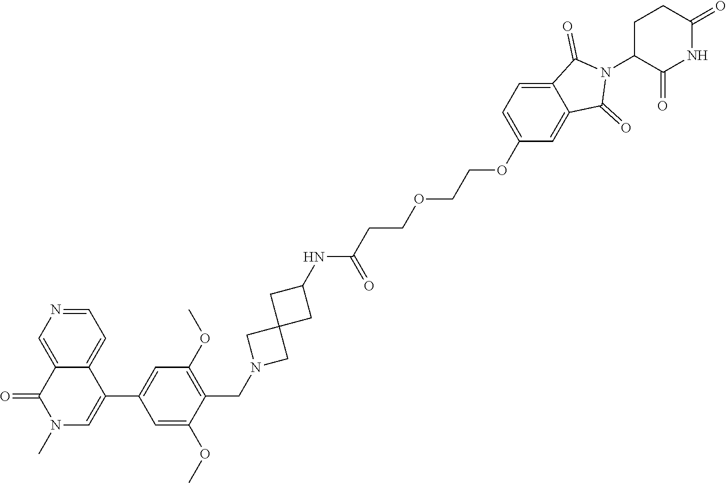 [SMILES: COc1cc(-c2cn(C)c(=O)c3cnccc23)cc(OC)c1CN1CC2(CC(NC(=O)CCOCCOc3ccc4c(c3)C(=O)N(C3CCC(=O)NC3=O)C4=O)C2)C1] |
| D102 | 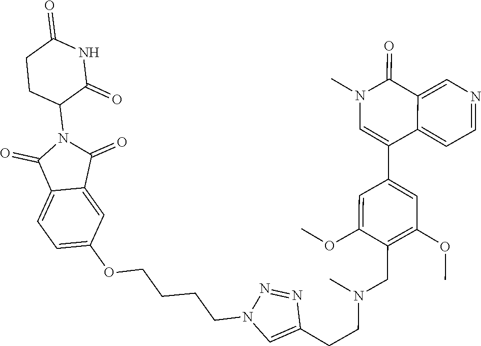 [SMILES: COc1cc(-c2cn(C)c(=O)c3cnccc23)cc(OC)c1CN(C)CCc1cn(CCCCOc2ccc3c(c2)C(=O)N(C2CCC(=O)NC2=O)C3=O)nn1] |
| D103 | 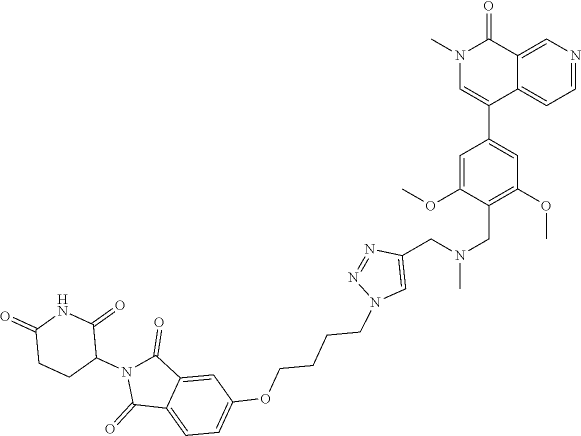 [SMILES: COc1cc(-c2cn(C)c(=O)c3cnccc23)cc(OC)c1CN(C)Cc1cn(CCCCOc2ccc3c(c2)C(=O)N(C2CCC(=O)NC2=O)C3=O)nn1] |
| D104 | 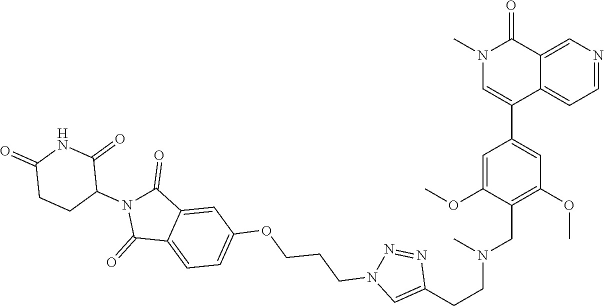 [SMILES: COc1cc(-c2cn(C)c(=O)c3cnccc23)cc(OC)c1CN(C)CCc1cn(CCCOc2ccc3c(c2)C(=O)N(C2CCC(=O)NC2=O)C3=O)nn1] |
| D105 | 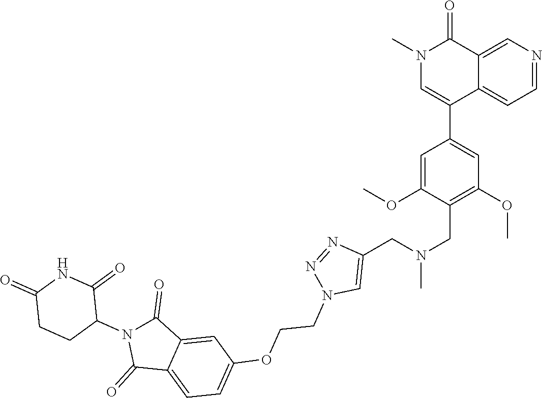 [SMILES: COc1cc(-c2cn(C)c(=O)c3cnccc23)cc(OC)c1CN(C)Cc1cn(CCOc2ccc3c(c2)C(=O)N(C2CCC(=O)NC2=O)C3=O)nn1] |
| D106 | 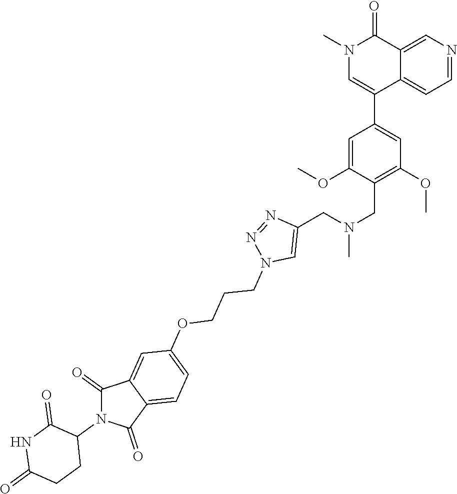 [SMILES: COc1cc(-c2cn(C)c(=O)c3cnccc23)cc(OC)c1CN(C)Cc1cn(CCCOc2ccc3c(c2)C(=O)N(C2CCC(=O)NC2=O)C3=O)nn1] |
| D107 | 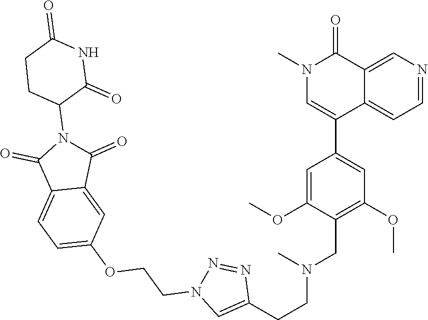 [SMILES: COc1cc(-c2cn(C)c(=O)c3cnccc23)cc(OC)c1CN(C)CCc1cn(CCOc2ccc3c(c2)C(=O)N(C2CCC(=O)NC2=O)C3=O)nn1] |
| D108 | 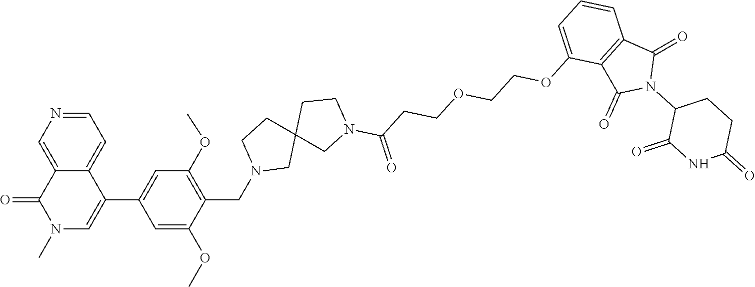 [SMILES: COc1cc(-c2cn(C)c(=O)c3cnccc23)cc(OC)c1CN1CCC2(CCN(C(=O)CCOCCOc3cccc4c3C(=O)N(C3CCC(=O)NC3=O)C4=O)C2)C1] |
| D109 | 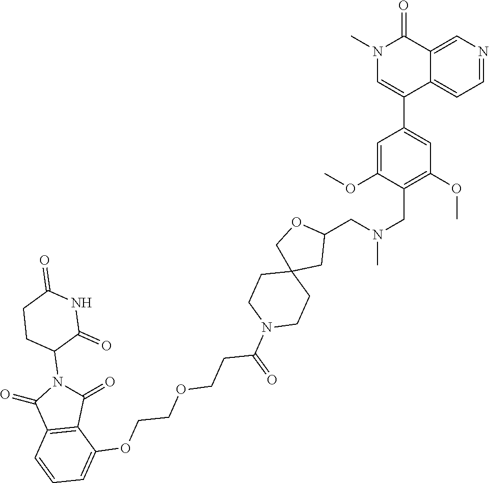 [SMILES: COc1cc(-c2cn(C)c(=O)c3cnccc23)cc(OC)c1CN(C)CC1CC2(CCN(C(=O)CCOCCOc3cccc4c3C(=O)N(C3CCC(=O)NC3=O)C4=O)CC2)CO1] |
| D110 | 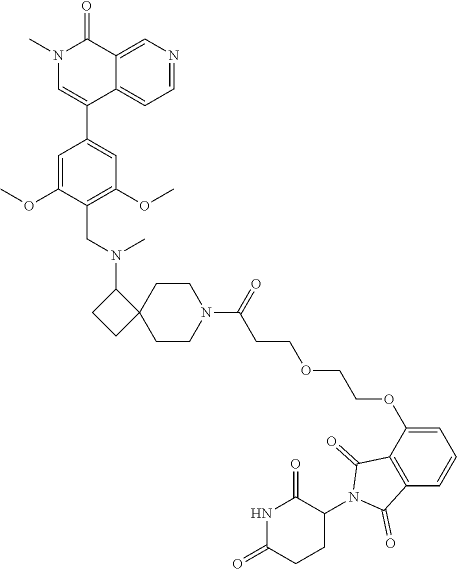 [SMILES: COc1cc(-c2cn(C)c(=O)c3cnccc23)cc(OC)c1CN(C)C1CCC12CCN(C(=O)CCOCCOc1cccc3c1C(=O)N(C1CCC(=O)NC1=O)C3=O)CC2] |
| D111 | 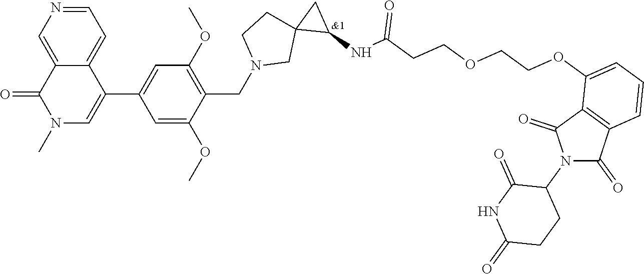 [SMILES: COc1cc(-c2cn(C)c(=O)c3cnccc23)cc(OC)c1CN1CCC2(C[C@H]2NC(=O)CCOCCOc2cccc3c2C(=O)N(C2CCC(=O)NC2=O)C3=O)C1] |
| D112 | 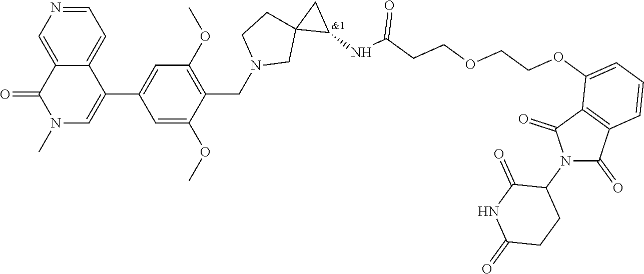 [SMILES: COc1cc(-c2cn(C)c(=O)c3cnccc23)cc(OC)c1CN1CCC2(C[C@@H]2NC(=O)CCOCCOc2cccc3c2C(=O)N(C2CCC(=O)NC2=O)C3=O)C1] |
| D113 | 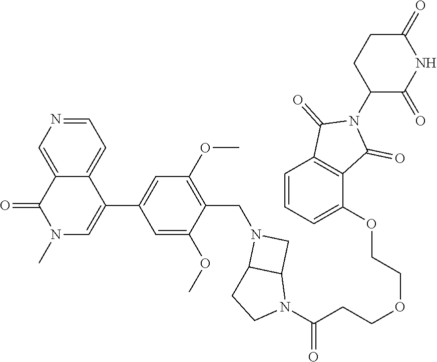 [SMILES: COc1cc(-c2cn(C)c(=O)c3cnccc23)cc(OC)c1CN1CC2C1CCN2C(=O)CCOCCOc1cccc2c1C(=O)N(C1CCC(=O)NC1=O)C2=O] |
| D114 | 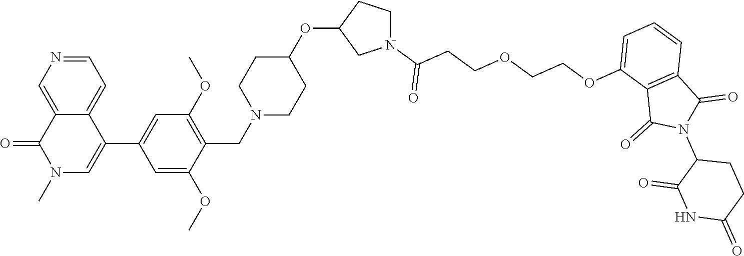 [SMILES: COc1cc(-c2cn(C)c(=O)c3cnccc23)cc(OC)c1CN1CCC(OC2CCN(C(=O)CCOCCOc3cccc4c3C(=O)N(C3CCC(=O)NC3=O)C4=O)C2)CC1] |
| D115 | 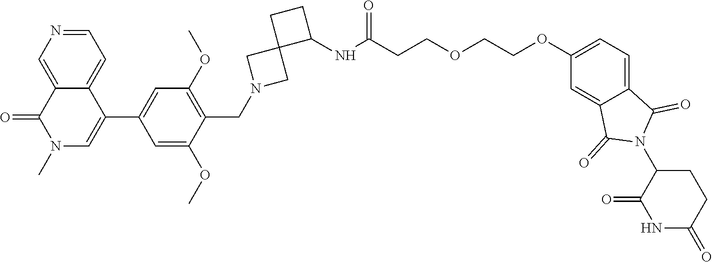 [SMILES: COc1cc(-c2cn(C)c(=O)c3cnccc23)cc(OC)c1CN1CC2(CCC2NC(=O)CCOCCOc2ccc3c(c2)C(=O)N(C2CCC(=O)NC2=O)C3=O)C1] |
| D116 | 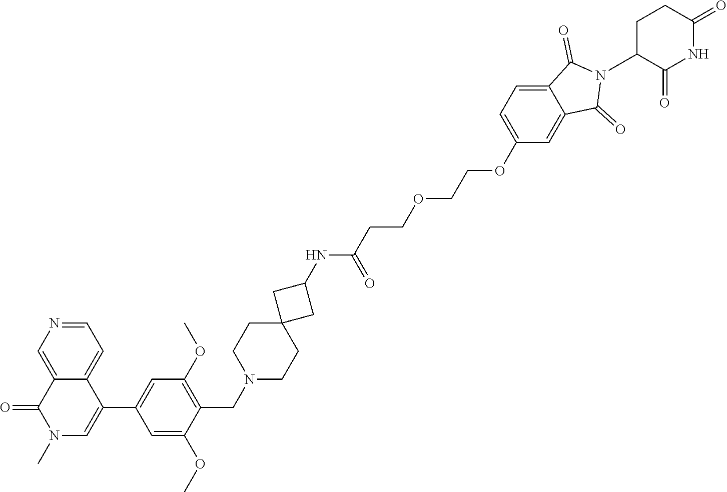 [SMILES: COc1cc(-c2cn(C)c(=O)c3cnccc23)cc(OC)c1CN1CCC2(CC1)CC(NC(=O)CCOCCOc1ccc3c(c1)C(=O)N(C1CCC(=O)NC1=O)C3=O)C2] |
| D117 | 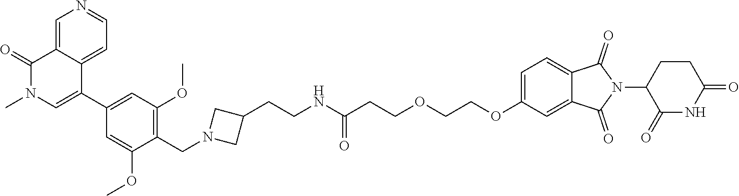 [SMILES: COc1cc(-c2cn(C)c(=O)c3cnccc23)cc(OC)c1CN1CC(CCNC(=O)CCOCCOc2ccc3c(c2)C(=O)N(C2CCC(=O)NC2=O)C3=O)C1] |
| D118 | 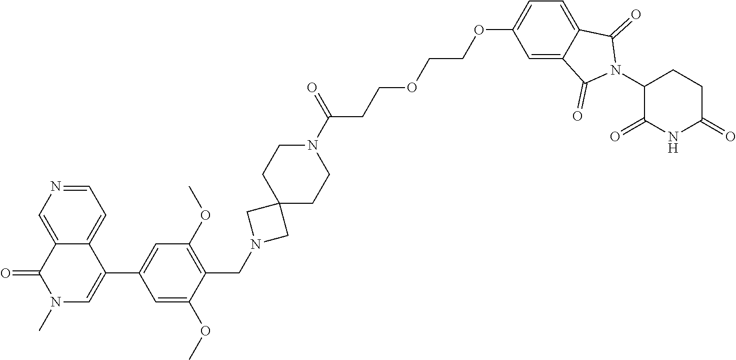 [SMILES: COc1cc(-c2cn(C)c(=O)c3cnccc23)cc(OC)c1CN1CC2(CCN(C(=O)CCOCCOc3ccc4c(c3)C(=O)N(C3CCC(=O)NC3=O)C4=O)CC2)C1] |
| D119 | 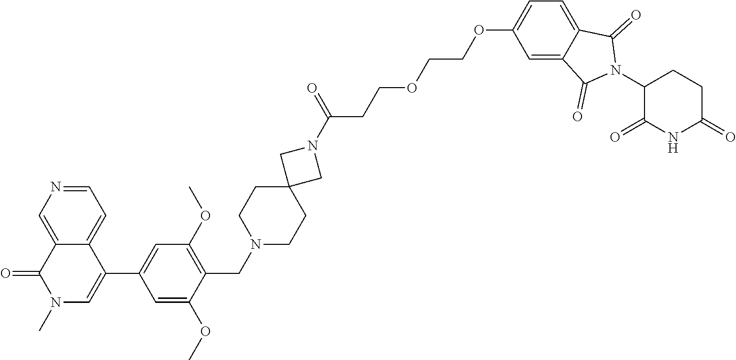 [SMILES: COc1cc(-c2cn(C)c(=O)c3cnccc23)cc(OC)c1CN1CCC2(CC1)CN(C(=O)CCOCCOc1ccc3c(c1)C(=O)N(C1CCC(=O)NC1=O)C3=O)C2] |
| D120 | 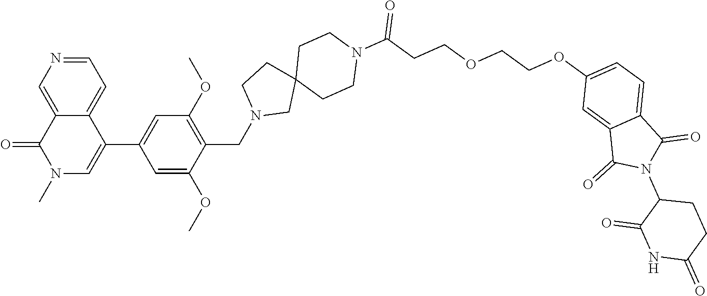 [SMILES: COc1cc(-c2cn(C)c(=O)c3cnccc23)cc(OC)c1CN1CCC2(CCN(C(=O)CCOCCOc3ccc4c(c3)C(=O)N(C3CCC(=O)NC3=O)C4=O)CC2)C1] |
| D121 | 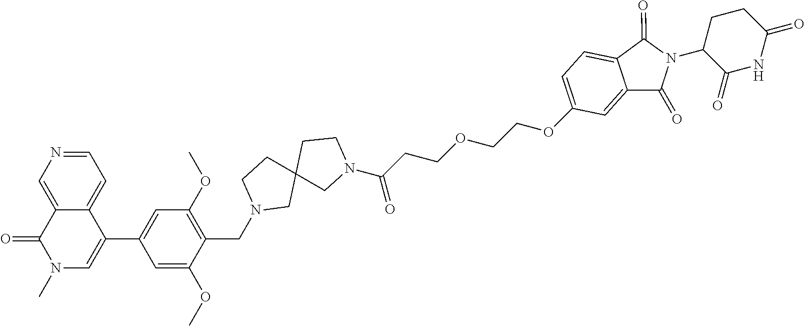 [SMILES: COc1cc(-c2cn(C)c(=O)c3cnccc23)cc(OC)c1CN1CCC2(CCN(C(=O)CCOCCOc3ccc4c(c3)C(=O)N(C3CCC(=O)NC3=O)C4=O)C2)C1] |
| D122 | 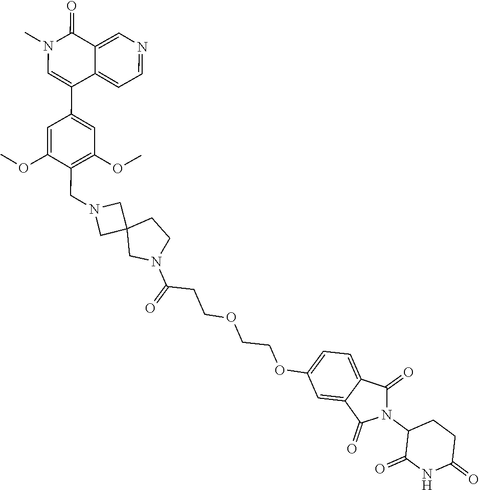 [SMILES: COc1cc(-c2cn(C)c(=O)c3cnccc23)cc(OC)c1CN1CC2(CCN(C(=O)CCOCCOc3ccc4c(c3)C(=O)N(C3CCC(=O)NC3=O)C4=O)C2)C1] |
| D123 | 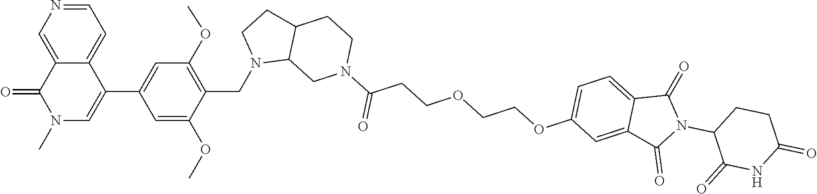 [SMILES: COc1cc(-c2cn(C)c(=O)c3cnccc23)cc(OC)c1CN1CCC2CCN(C(=O)CCOCCOc3ccc4c(c3)C(=O)N(C3CCC(=O)NC3=O)C4=O)CC21] |
| D124 | 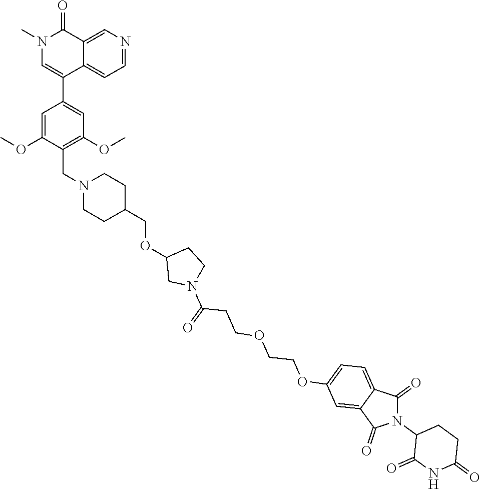 [SMILES: COc1cc(-c2cn(C)c(=O)c3cnccc23)cc(OC)c1CN1CCC(COC2CCN(C(=O)CCOCCOc3ccc4c(c3)C(=O)N(C3CCC(=O)NC3=O)C4=O)C2)CC1] |
| D125 | 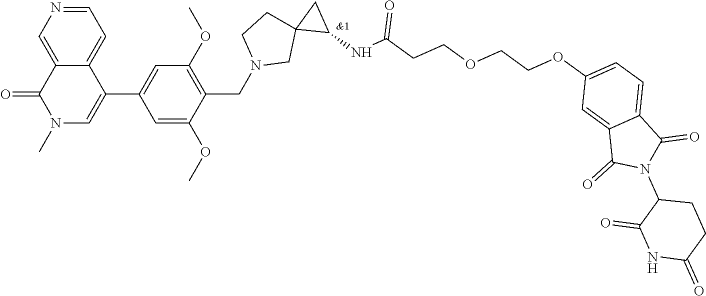 [SMILES: COc1cc(-c2cn(C)c(=O)c3cnccc23)cc(OC)c1CN1CCC2(C[C@@H]2NC(=O)CCOCCOc2ccc3c(c2)C(=O)N(C2CCC(=O)NC2=O)C3=O)C1] |
| D126 | 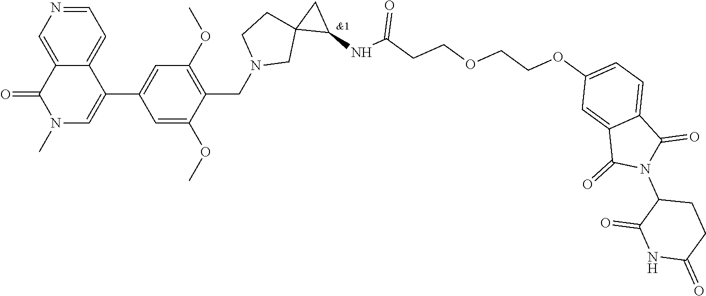 [SMILES: COc1cc(-c2cn(C)c(=O)c3cnccc23)cc(OC)c1CN1CCC2(C[C@H]2NC(=O)CCOCCOc2ccc3c(c2)C(=O)N(C2CCC(=O)NC2=O)C3=O)C1] |
| D127 | 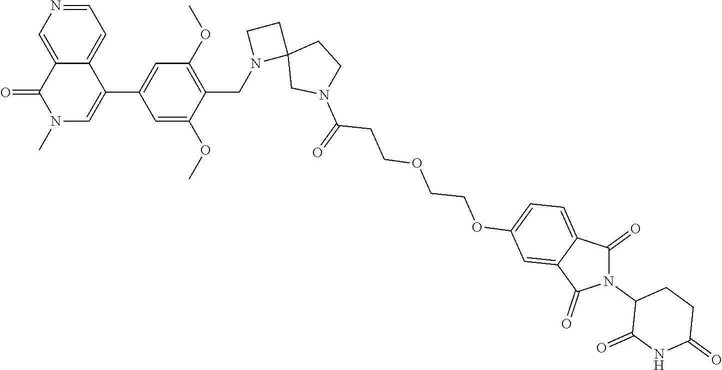 [SMILES: COc1cc(-c2cn(C)c(=O)c3cnccc23)cc(OC)c1CN1CCC12CCN(C(=O)CCOCCOc1ccc3c(c1)C(=O)N(C1CCC(=O)NC1=O)C3=O)C2] |
| D128 | 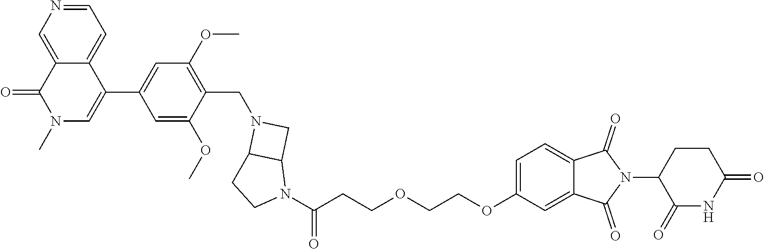 [SMILES: COc1cc(-c2cn(C)c(=O)c3cnccc23)cc(OC)c1CN1CC2C1CCN2C(=O)CCOCCOc1ccc2c(c1)C(=O)N(C1CCC(=O)NC1=O)C2=O] |
| D129 | 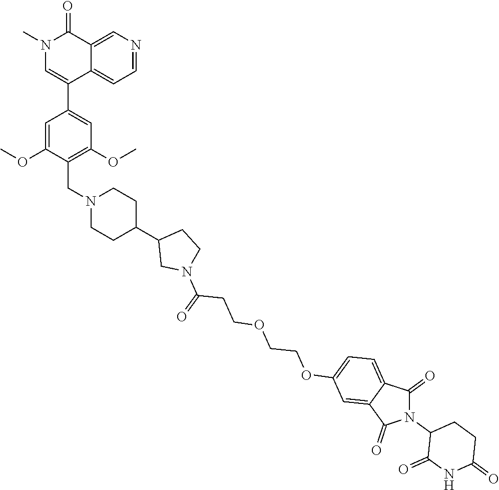 [SMILES: COc1cc(-c2cn(C)c(=O)c3cnccc23)cc(OC)c1CN1CCC(C2CCN(C(=O)CCOCCOc3ccc4c(c3)C(=O)N(C3CCC(=O)NC3=O)C4=O)C2)CC1] |
| D130 | 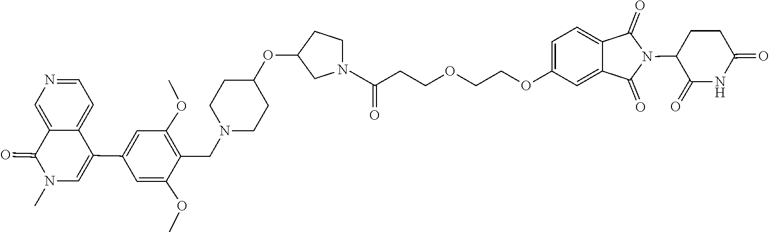 [SMILES: COc1cc(-c2cn(C)c(=O)c3cnccc23)cc(OC)c1CN1CCC(OC2CCN(CC(=O)CCOCCOc3ccc4c(c3)C(=O)N(C3CCC(=O)NC3=O)C4=O)C2)CC1] |
| D131 | 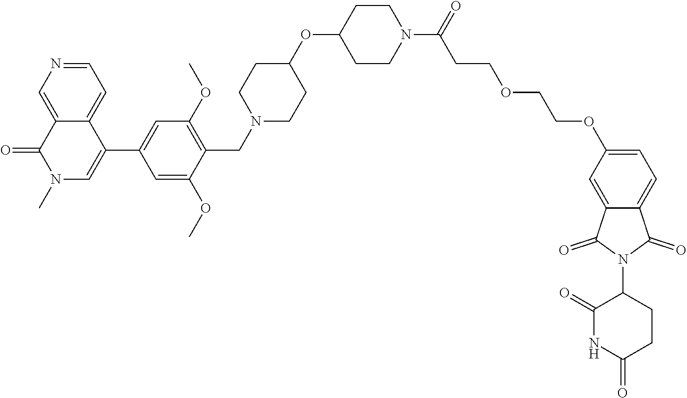 [SMILES: COc1cc(-c2cn(C)c(=O)c3cnccc23)cc(OC)c1CN1CCC(OC2CCN(C(=O)CCOCCOc3ccc4c(c3)C(=O)N(C3CCC(=O)NC3=O)C4=O)CC2)CC1] |
| D132 | 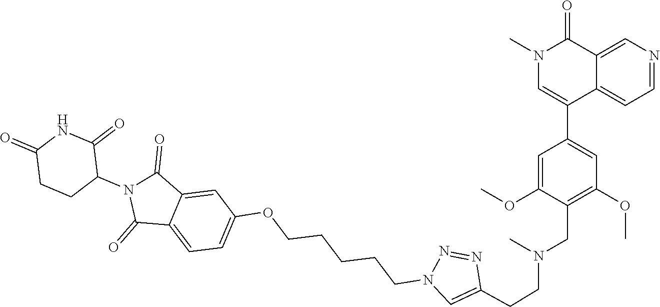 [SMILES: COc1cc(-c2cn(C)c(=O)c3cnccc23)cc(OC)c1CN(C)CCc1cn(CCCCCOc2ccc3c(c2)C(=O)N(C2CCC(=O)NC2=O)C3=O)nn1] |
| D133 | 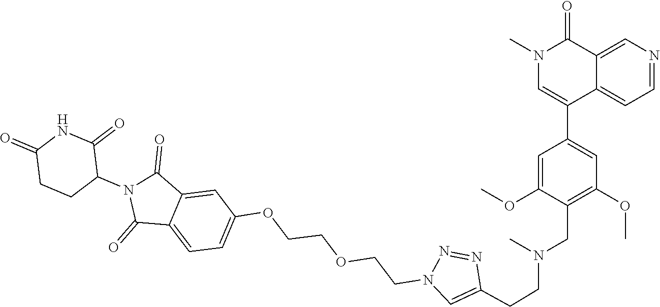 [SMILES: COc1cc(-c2cn(C)c(=O)c3cnccc23)cc(OC)c1CN(C)CCc1cn(CCOCCOc2ccc3c(c2)C(=O)N(C2CCC(=O)NC2=O)C3=O)nn1] |
| D134 | 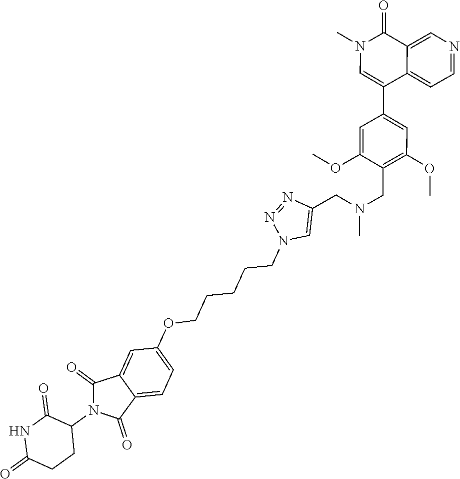 [SMILES: COc1cc(-c2cn(C)c(=O)c3cnccc23)cc(OC)c1CN(C)Cc1cn(CCCCCOc2ccc3c(c2)C(=O)N(C2CCC(=O)NC2=O)C3=O)nn1] |
| D135 | 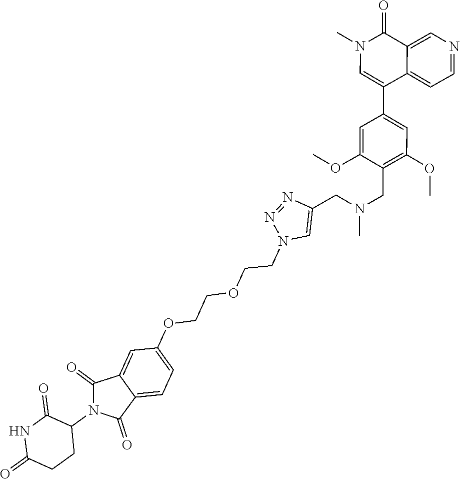 [SMILES: COc1cc(-c2cn(C)c(=O)c3cnccc23)cc(OC)c1CN(C)Cc1cn(CCOCCOc2ccc3c(c2)C(=O)N(C2CCC(=O)NC2=O)C3=O)nn1] |
| D136 | 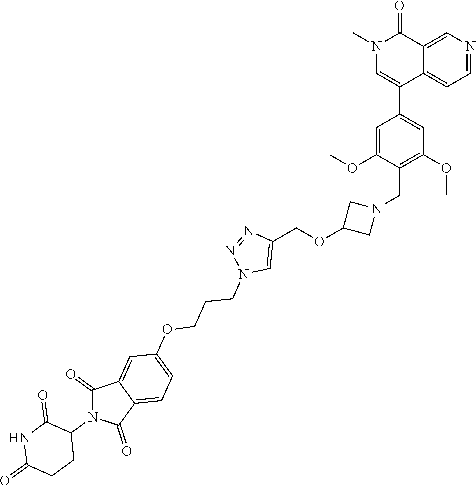 [SMILES: COc1cc(-c2cn(C)c(=O)c3cnccc23)cc(OC)c1CN1CC(OCc2cn(CCCOc3ccc4c(c3)C(=O)N(C3CCC(=O)NC3=O)C4=O)nn2)C1] |
| D137 | 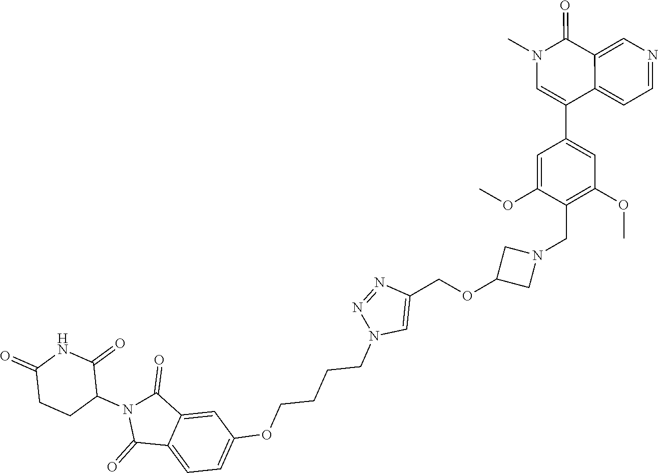 [SMILES: COc1cc(-c2cn(C)c(=O)c3cnccc23)cc(OC)c1CN1CC(OCc2cn(CCCCOc3ccc4c(c3)C(=O)N(C3CCC(=O)NC3=O)C4=O)nn2)C1] |
| D138 | 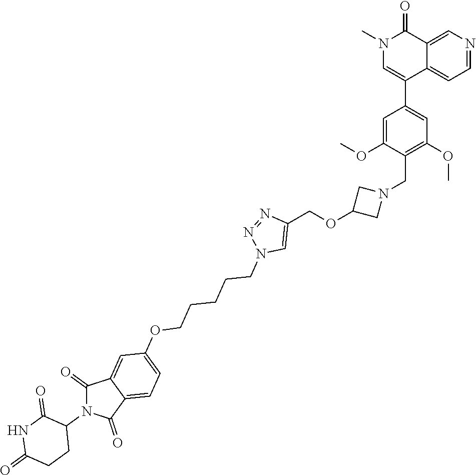 [SMILES: COc1cc(-c2cn(C)c(=O)c3cnccc23)cc(OC)c1CN1CC(OCc2cn(CCCCCOc3ccc4c(c3)C(=O)N(C3CCC(=O)NC3=O)C4=O)nn2)C1] |
| D139 | 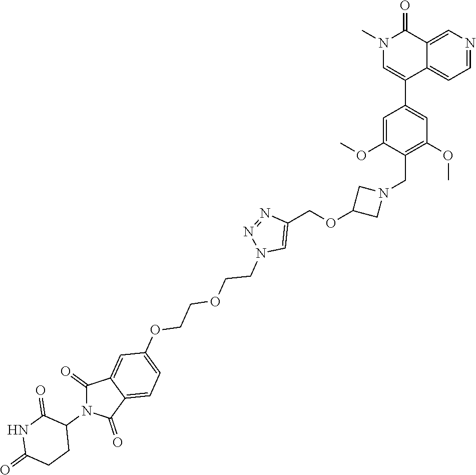 [SMILES: COc1cc(-c2cn(C)c(=O)c3cnccc23)cc(OC)c1CN1CC(OCc2cn(CCOCCOc3ccc4c(c3)C(=O)N(C3CCC(=O)NC3=O)C4=O)nn2)C1] |
| D140 | 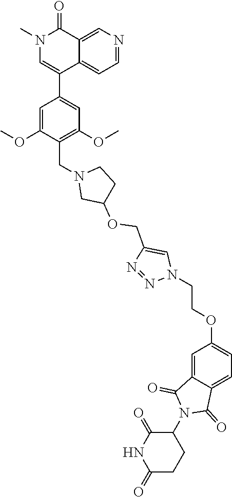 [SMILES: COc1cc(-c2cn(C)c(=O)c3cnccc23)cc(OC)c1CN1CCC(OCc2cn(CCOc3ccc4c(c3)C(=O)N(C3CCC(=O)NC3=O)C4=O)nn2)C1] |
| D141 | 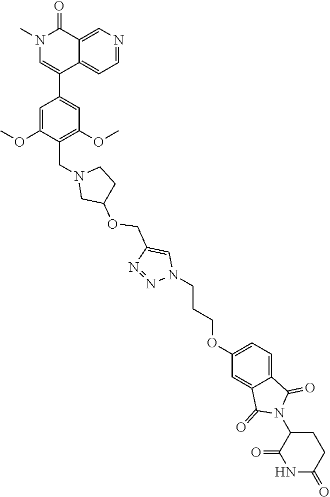 [SMILES: COc1cc(-c2cn(C)c(=O)c3cnccc23)cc(OC)c1CN1CCC(OCc2cn(CCCOc3ccc4c(c3)C(=O)N(C3CCC(=O)NC3=O)C4=O)nn2)C1] |
| D142 | 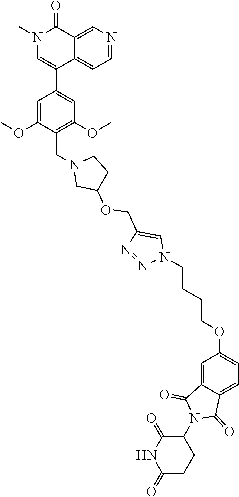 [SMILES: COc1cc(-c2cn(C)c(=O)c3cnccc23)cc(OC)c1CN1CCC(OCc2cn(CCCCOc3ccc4c(c3)C(=O)N(C3CCC(=O)NC3=O)C4=O)nn2)C1] |
| D143 | 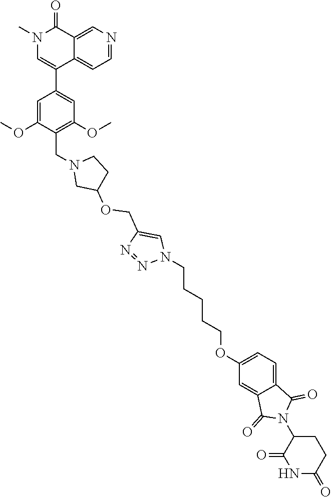 [SMILES: COc1cc(-c2cn(C)c(=O)c3cnccc23)cc(OC)c1CN1CCC(OCc2cn(CCCCCOc3ccc4c(c3)C(=O)N(C3CCC(=O)NC3=O)C4=O)nn2)C1] |
| D144 | 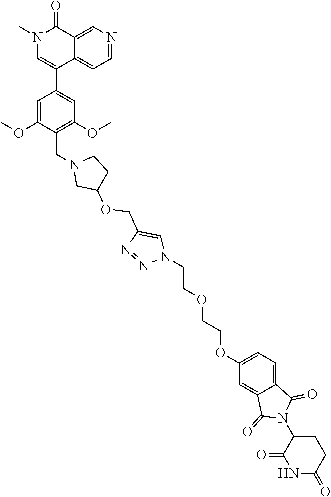 [SMILES: COc1cc(-c2cn(C)c(=O)c3cnccc23)cc(OC)c1CN1CCC(OCc2cn(CCOCCOc3ccc4c(c3)C(=O)N(C3CCC(=O)NC3=O)C4=O)nn2)C1] |
| D145 | 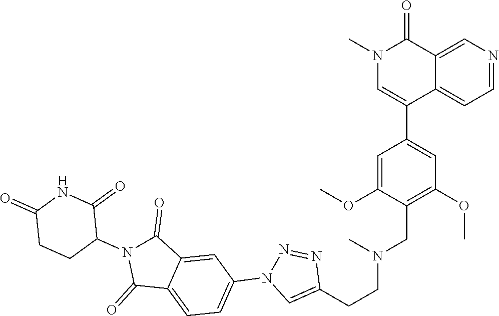 [SMILES: COc1cc(-c2cn(C)c(=O)c3cnccc23)cc(OC)c1CN(C)CCc1cn(-c2ccc3c(c2)C(=O)N(C2CCC(=O)NC2=O)C3=O)nn1] |
| D146 | 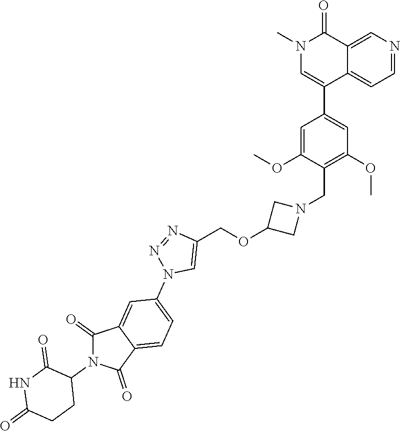 [SMILES: COc1cc(-c2cn(C)c(=O)c3cnccc23)cc(OC)c1CN1CC(OCc2cn(-c3ccc4c(c3)C(=O)N(C3CCC(=O)NC3=O)C4=O)nn2)C1] |
| D147 | 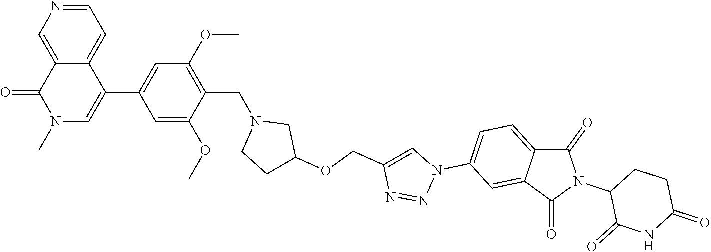 [SMILES: COc1cc(-c2cn(C)c(=O)c3cnccc23)cc(OC)c1CN1CCC(OCc2cn(-c3ccc4c(c3)C(=O)N(C3CCC(=O)NC3=O)C4=O)nn2)C1] |
| D148 | 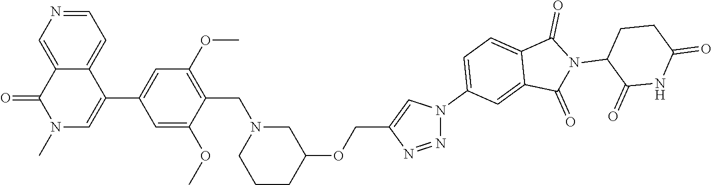 [SMILES: COc1cc(-c2cn(C)c(=O)c3cnccc23)cc(OC)c1CN1CCCC(OCc2cn(-c3ccc4c(c3)C(=O)N(C3CCC(=O)NC3=O)C4=O)nn2)C1] |
| D149 | 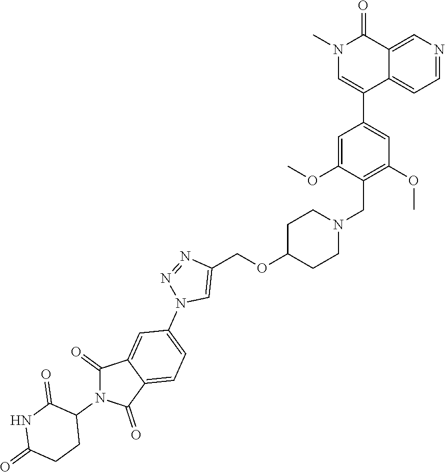 [SMILES: COc1cc(-c2cn(C)c(=O)c3cnccc23)cc(OC)c1CN1CCC(OCc2cn(-c3ccc4c(c3)C(=O)N(C3CCC(=O)NC3=O)C4=O)nn2)CC1] |
| D150 | 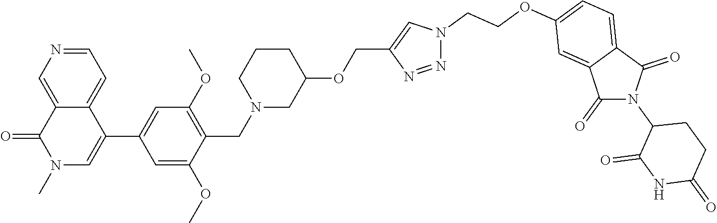 [SMILES: COc1cc(-c2cn(C)c(=O)c3cnccc23)cc(OC)c1CN1CCCC(OCc2cn(CCOc3ccc4c(c3)C(=O)N(C3CCC(=O)NC3=O)C4=O)nn2)C1] |
| D151 | 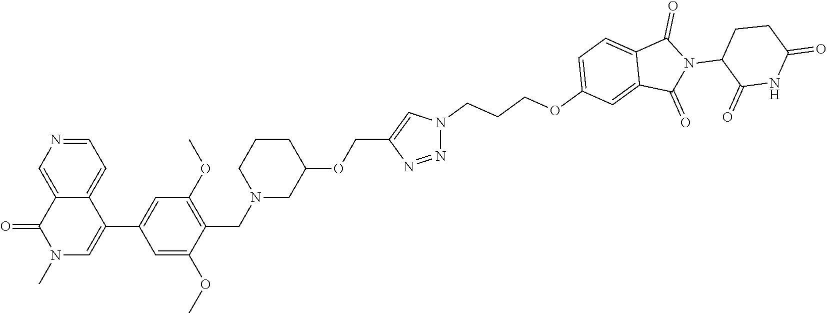 [SMILES: COc1cc(-c2cn(C)c(=O)c3cnccc23)cc(OC)c1CN1CCCC(OCc2cn(CCCOc3ccc4c(c3)C(=O)N(C3CCC(=O)NC3=O)C4=O)nn2)C1] |
| D152 | 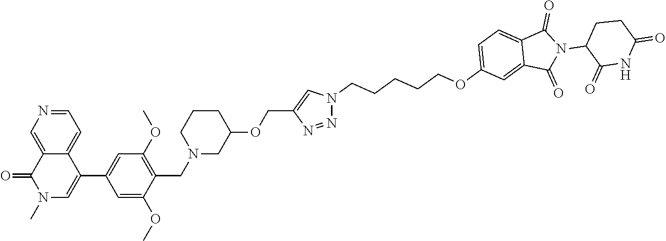 [SMILES: COc1cc(-c2cn(C)c(=O)c3cnccc23)cc(OC)c1CN1CCCC(OCc2cn(CCCCCOc3ccc4c(c3)C(=O)N(C3CCC(=O)NC3=O)C4=O)nn2)C1] |
| D153 | 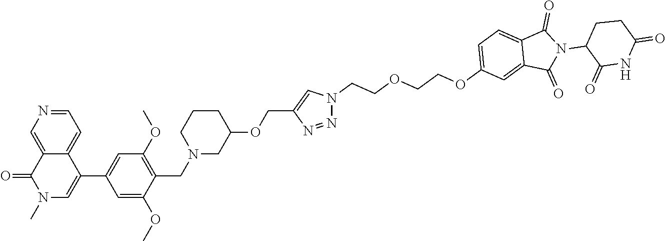 [SMILES: COc1cc(-c2cn(C)c(=O)c3cnccc23)cc(OC)c1CN1CCCC(OCc2cn(CCOCCOc3ccc4c(c3)C(=O)N(C3CCC(=O)NC3=O)C4=O)nn2)C1] |
| D154 | 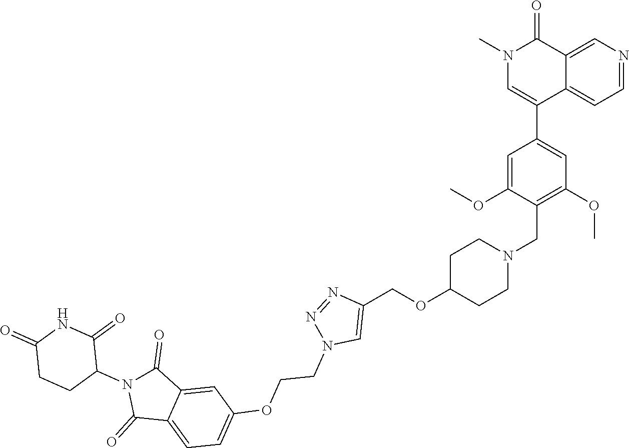 [SMILES: COc1cc(-c2cn(C)c(=O)c3cnccc23)cc(OC)c1CN1CCC(OCc2cn(CCOc3ccc4c(c3)C(=O)N(C3CCC(=O)NC3=O)C4=O)nn2)CC1] |
| D155 | 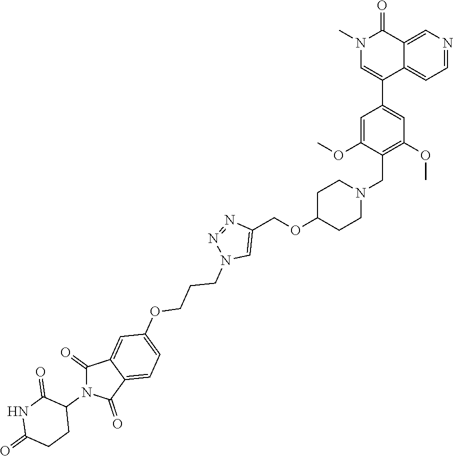 [SMILES: COc1cc(-c2cn(C)c(=O)c3cnccc23)cc(OC)c1CN1CCC(OCc2cn(CCCOc3ccc4c(c3)C(=O)N(C3CCC(=O)NC3=O)C4=O)nn2)CC1] |
| D156 | 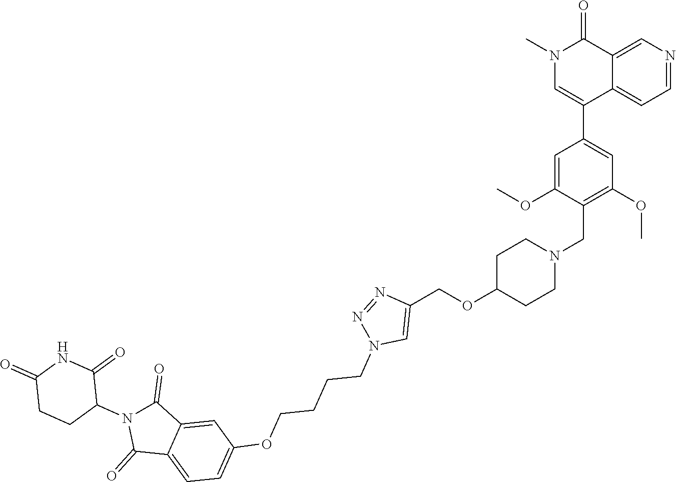 [SMILES: COc1cc(-c2cn(C)c(=O)c3cnccc23)cc(OC)c1CN1CCC(OCc2cn(CCCCOc3ccc4c(c3)C(=O)N(C3CCC(=O)NC3=O)C4=O)nn2)CC1] |
| D157 | 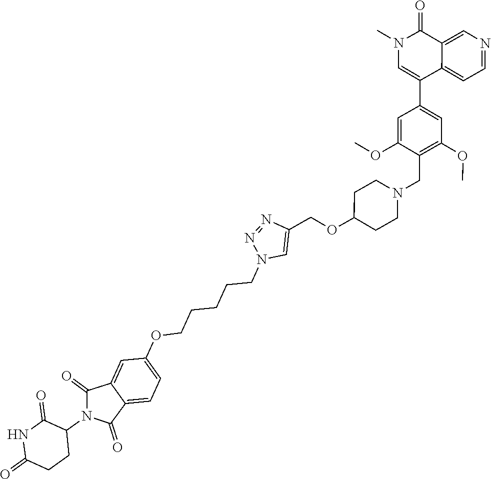 [SMILES: COc1cc(-c2cn(C)c(=O)c3cnccc23)cc(OC)c1CN1CCC(OCc2cn(CCCCCOc3ccc4c(c3)C(=O)N(C3CCC(=O)NC3=O)C4=O)nn2)CC1] |
| D158 | 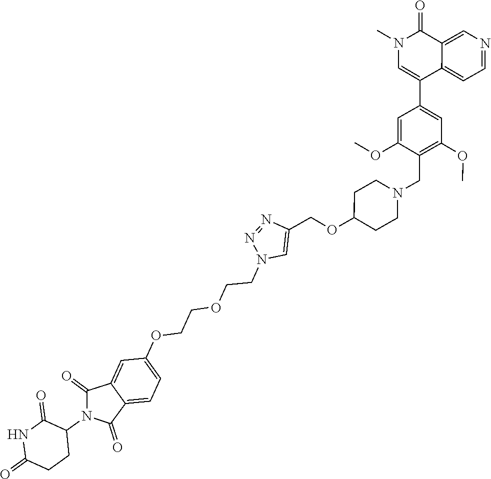 [SMILES: COc1cc(-c2cn(C)c(=O)c3cnccc23)cc(OC)c1CN1CCC(OCc2cn(CCOCCOc3ccc4c(c3)C(=O)N(C3CCC(=O)NC3=O)C4=O)nn2)CC1] |
| D159 | 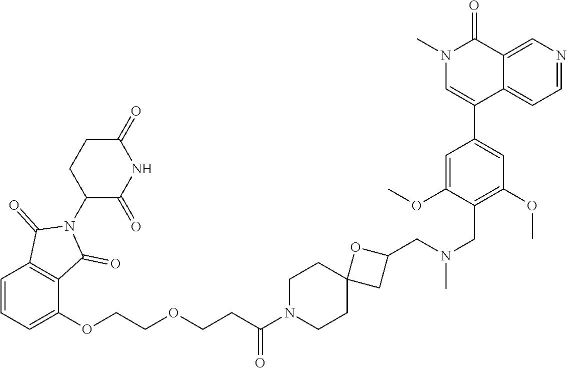 [SMILES: COc1cc(-c2cn(C)c(=O)c3cnccc23)cc(OC)c1CN(C)CC1CC2(CCN(C(=O)CCOCCOc3cccc4c3C(=O)N(C3CCC(=O)NC3=O)C4=O)CC2)O1] |
| D160 | 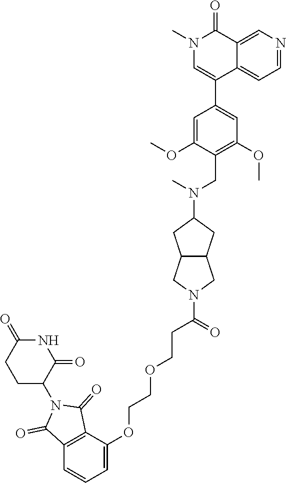 [SMILES: COc1cc(-c2cn(C)c(=O)c3cnccc23)cc(OC)c1CN(C)C1CC2CN(C(=O)CCOCCOc3cccc4c3C(=O)N(C3CCC(=O)NC3=O)C4=O)CC2C1] |
| D161 | 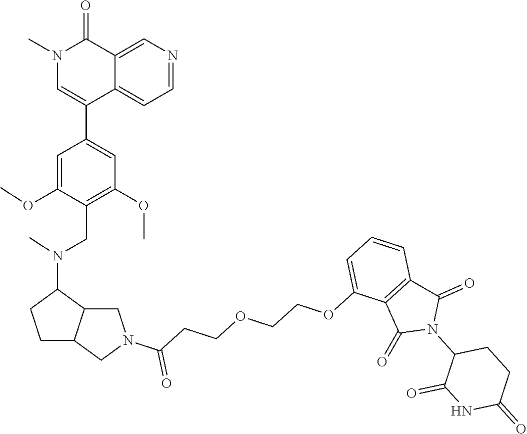 [SMILES: COc1cc(-c2cn(C)c(=O)c3cnccc23)cc(OC)c1CN(C)C1CCC2CN(C(=O)CCOCCOc3cccc4c3C(=O)N(C3CCC(=O)NC3=O)C4=O)CC21] |
| D162 | 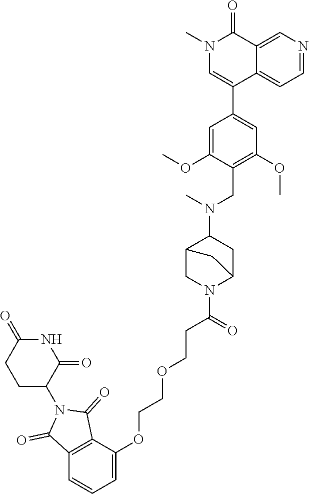 [SMILES: COc1cc(-c2cn(C)c(=O)c3cnccc23)cc(OC)c1CN(C)C1CC2CC1CN2C(=O)CCOCCOc1cccc2c1C(=O)N(C1CCC(=O)NC1=O)C2=O] |
| D163 | 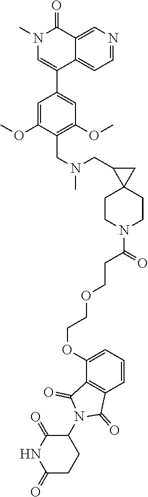 [SMILES: COc1cc(-c2cn(C)c(=O)c3cnccc23)cc(OC)c1CN(C)CC1CC12CCN(C(=O)CCOCCOc1cccc3c1C(=O)N(C1CCC(=O)NC1=O)C3=O)CC2] |
| D164 | 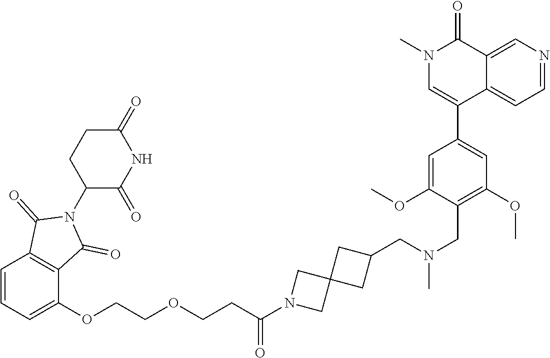 [SMILES: COc1cc(-c2cn(C)c(=O)c3cnccc23)cc(OC)c1CN(C)CC1CC2(C1)CN(C(=O)CCOCCOc1cccc3c1C(=O)N(C1CCC(=O)NC1=O)C3=O)C2] |
| D165 | 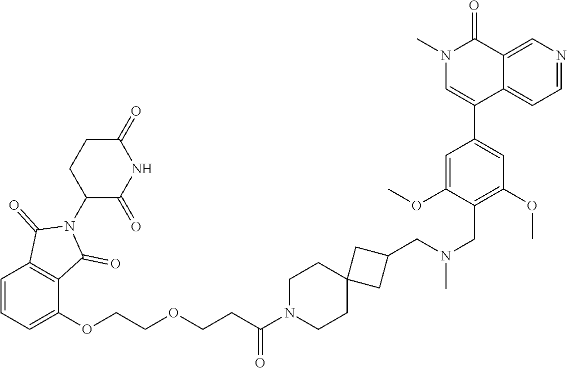 [SMILES: COc1cc(-c2cn(C)c(=O)c3cnccc23)cc(OC)c1CN(C)CC1CC2(CCN(C(=O)CCOCCOc3cccc4c3C(=O)N(C3CCC(=O)NC3=O)C4=O)CC2)C1] |
| D166 | 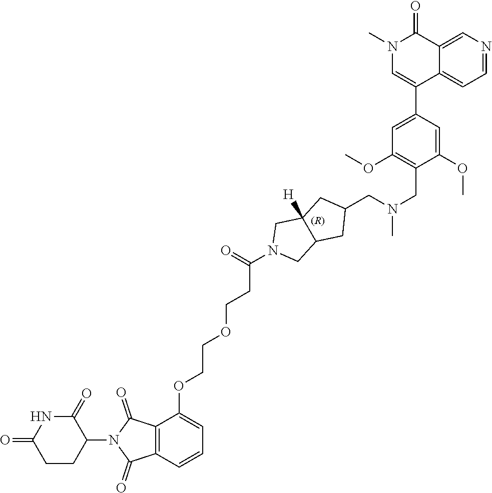 |
| D167 | 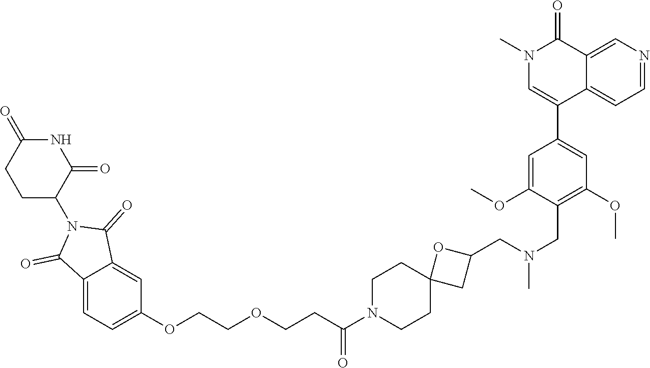 [SMILES: COc1cc(-c2cn(C)c(=O)c3cnccc23)cc(OC)c1CN(C)CC1CC2(CCN(C(=O)CCOCCOc3ccc4c(c3)C(=O)N(C3CCC(=O)NC3=O)C4=O)CC2)O1] |
| D168 | 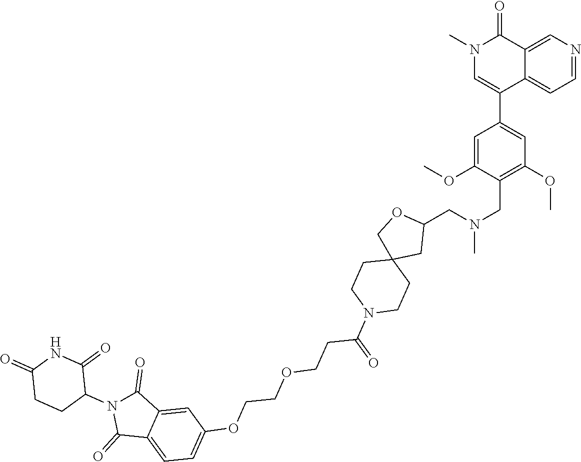 [SMILES: COc1cc(-c2cn(C)c(=O)c3cnccc23)cc(OC)c1CN(C)CC1CC2(CCN(C(=O)CCOCCOc3ccc4c(c3)C(=O)N(C3CCC(=O)NC3=O)C4=O)CC2)CO1] |
| D169 | 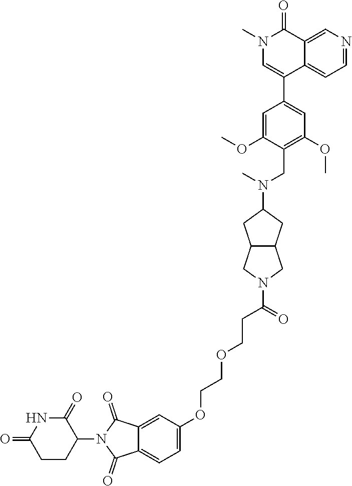 [SMILES: COc1cc(-c2cn(C)c(=O)c3cnccc23)cc(OC)c1CN(C)C1CC2CN(C(=O)CCOCCOc3ccc4c(c3)C(=O)N(C3CCC(=O)NC3=O)C4=O)CC2C1] |
| D170 | 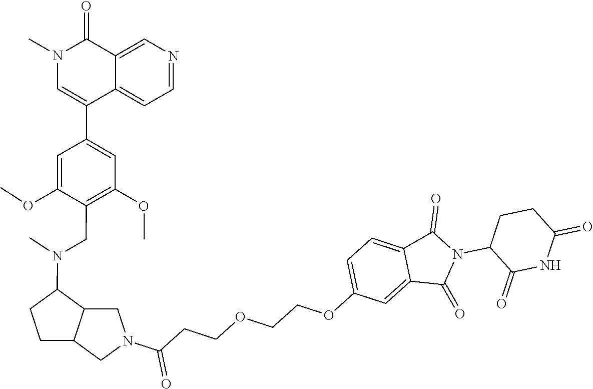 [SMILES: COc1cc(-c2cn(C)c(=O)c3cnccc23)cc(OC)c1CN(C)C1CCC2CN(C(=O)CCOCCOc3ccc4c(c3)C(=O)N(C3CCC(=O)NC3=O)C4=O)CC21] |
| D171 | 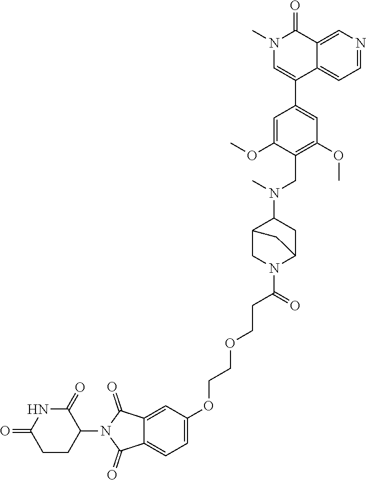 [SMILES: COc1cc(-c2cn(C)c(=O)c3cnccc23)cc(OC)c1CN(C)C1CC2CC1CN2C(=O)CCOCCOc1ccc2c(c1)C(=O)N(C1CCC(=O)NC1=O)C2=O] |
| D172 | 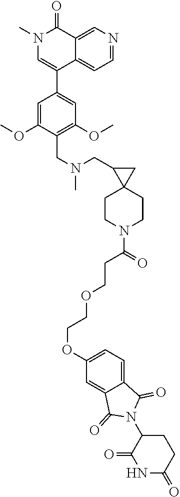 [SMILES: COc1cc(-c2cn(C)c(=O)c3cnccc23)cc(OC)c1CN(C)CC1CC12CCN(C(=O)CCOCCOc1ccc3c(c1)C(=O)N(C1CCC(=O)NC1=O)C3=O)CC2] |
| D173 | 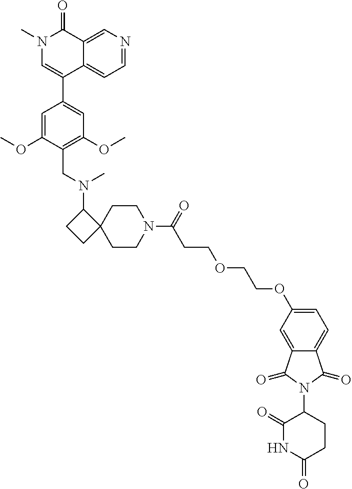 [SMILES: COc1cc(-c2cn(C)c(=O)c3cnccc23)cc(OC)c1CN(C)C1CCC12CCN(C(=O)CCOCCOc1ccc3c(c1)C(=O)N(C1CCC(=O)NC1=O)C3=O)CC2] |
| D174 | 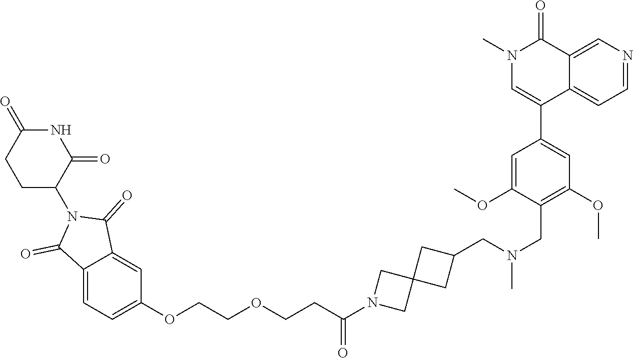 [SMILES: COc1cc(-c2cn(C)c(=O)c3cnccc23)cc(OC)c1CN(C)CC1CC2(C1)CN(C(=O)CCOCCOc1ccc3c(c1)C(=O)N(C1CCC(=O)NC1=O)C3=O)C2] |
| D175 | 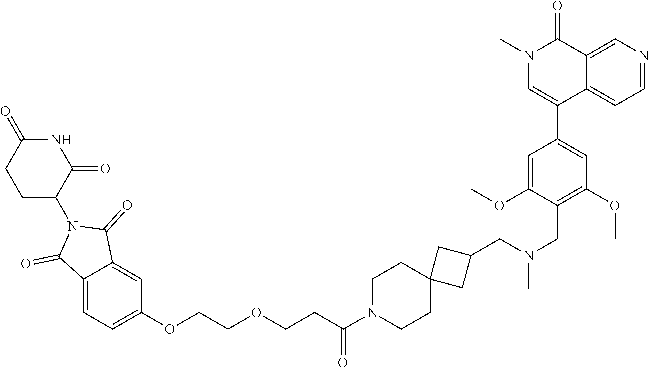 [SMILES: COc1cc(-c2cn(C)c(=O)c3cnccc23)cc(OC)c1CN(C)CC1CC2(CCN(C(=O)CCOCCOc3ccc4c(c3)C(=O)N(C3CCC(=O)NC3=O)C4=O)CC2)C1] |
| D176 | 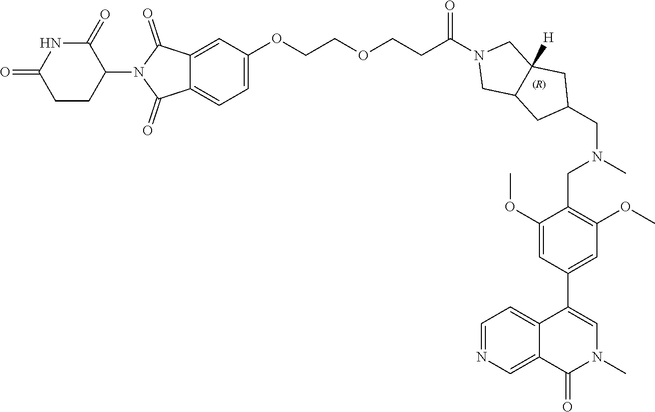 |
| D177 | 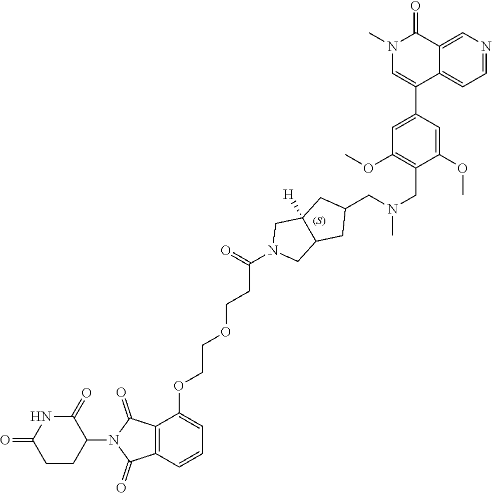 |

TABLE 1B
Compounds D178-D371 of the Disclosure

| Com- | |
|---|---|
| pound | |
| No. | Structure |
| D178 | 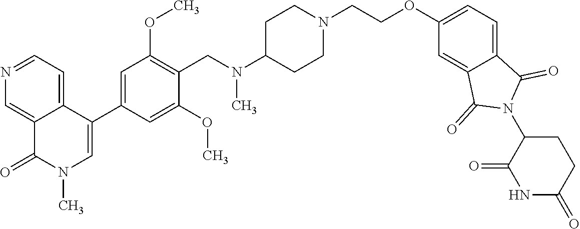 [SMILES: COc1cc(-c2cn(C)c(=O)c3cnccc23)cc(OC)c1CN(C)C1CCN(CCOc2ccc3c(c2)C(=O)N(C2CCC(=O)NC2=O)C3=O)CC1] |
| D179 | 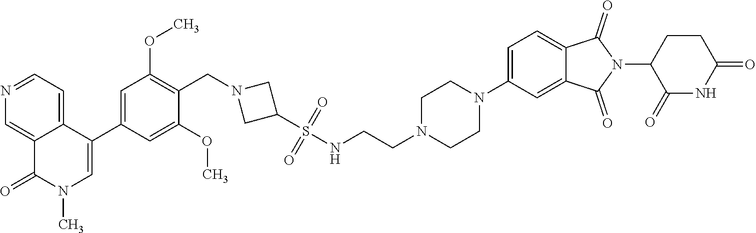 [SMILES: COc1cc(-c2cn(C)c(=O)c3cnccc23)cc(OC)c1CN1CC(S(=O)(=O)NCCN2CCN(c3ccc4c(c3)C(=O)N(C3CCC(=O)NC3=O)C4=O)CC2)C1] |
| D180 | 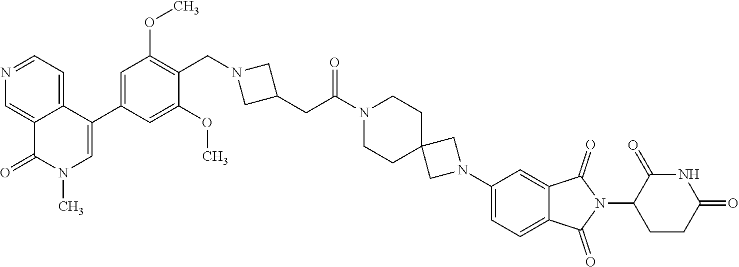 [SMILES: COc1cc(-c2cn(C)c(=O)c3cnccc23)cc(OC)c1CN1CC(CC(=O)N2CCC3(CC2)CN(c2ccc4c(c2)C(=O)N(C2CCC(=O)NC2=O)C4=O)C3)C1] |
| D181 | 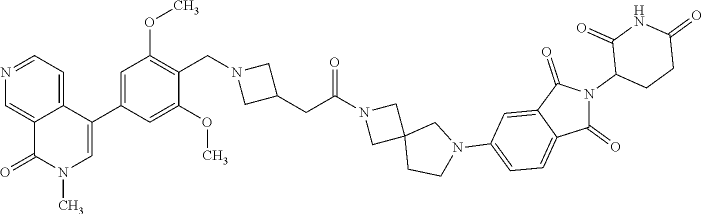 [SMILES: COc1cc(-c2cn(C)c(=O)c3cnccc23)cc(OC)c1CN1CC(CC(=O)N2CC3(CCN(c4ccc5c(c4)C(=O)N(C4CCC(=O)NC4=O)C5=O)C3)C2)C1] |
| D182 | 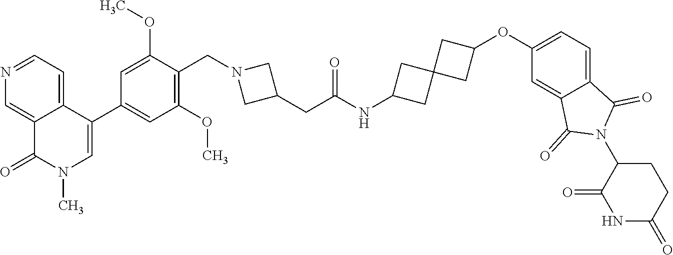 [SMILES: COc1cc(-c2cn(C)c(=O)c3cnccc23)cc(OC)c1CN1CC(CC(=O)NC2CC3(C2)CC(Oc2ccc4c(c2)C(=O)N(C2CCC(=O)NC2=O)C4=O)C3)C1] |
| D183 | 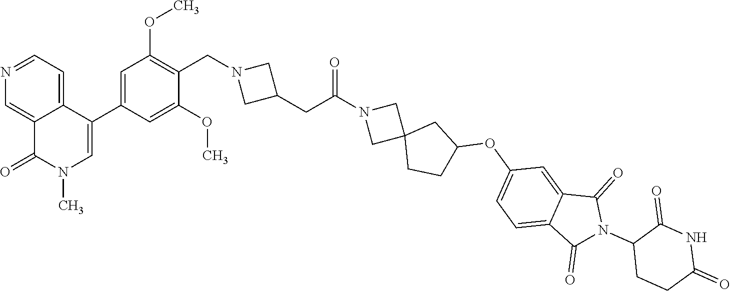 [SMILES: COc1cc(-c2cn(C)c(=O)c3cnccc23)cc(OC)c1CN1CC(CC(=O)N2CC3(CCC(Oc4ccc5c(c4)C(=O)N(C4CCC(=O)NC4=O)C5=O)C3)C2)C1] |
| D184 | 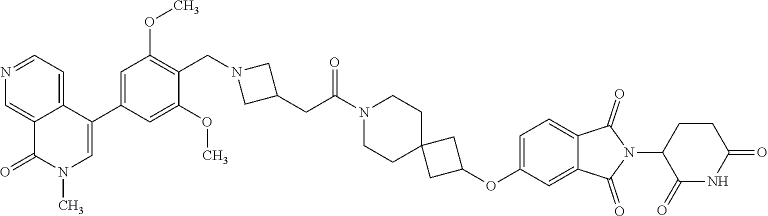 [SMILES: COc1cc(-c2cn(C)c(=O)c3cnccc23)cc(OC)c1CN1CC(CC(=O)N2CCC3(CC2)CC(Oc2ccc4c(c2)C(=O)N(C2CCC(=O)NC2=O)C4=O)C3)C1] |
| D185 | 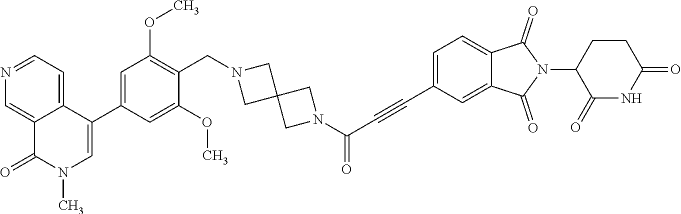 [SMILES: COc1cc(-c2cn(C)c(=O)c3cnccc23)cc(OC)c1CN1CC2(C1)CN(C(=O)C#Cc1ccc3c(c1)C(=O)N(C1CCC(=O)NC1=O)C3=O)C2] |
| D186 | 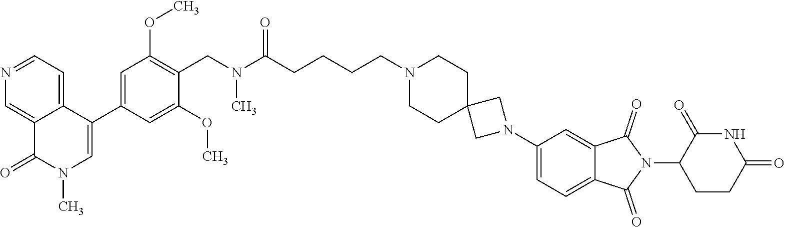 [SMILES: COc1cc(-c2cn(C)c(=O)c3cnccc23)cc(OC)c1CN(C)C(=O)CCCCN1CCC2(CC1)CN(c1ccc3c(c1)C(=O)N(C1CCC(=O)NC1=O)C3=O)C2] |
| D187 | 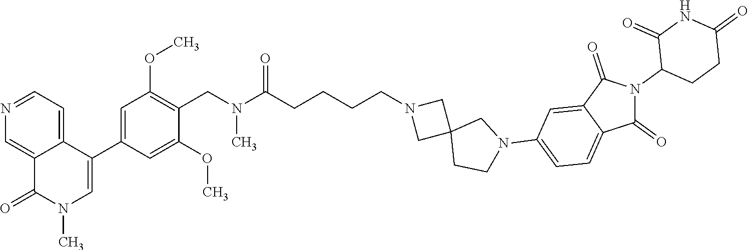 [SMILES: COc1cc(-c2cn(C)c(=O)c3cnccc23)cc(OC)c1CN(C)C(=O)CCCCN1CC2(CCN(c3ccc4c(c3)C(=O)N(C3CCC(=O)NC3=O)C4=O)C2)C1] |
| D188 | 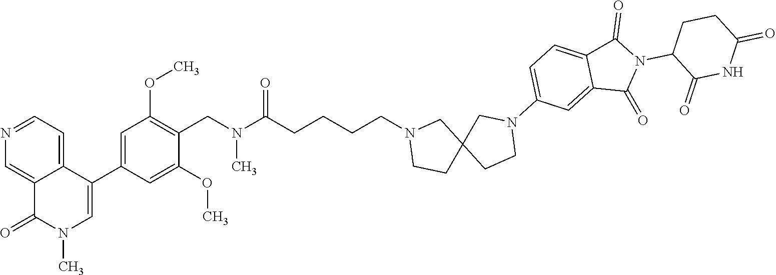 [SMILES: COc1cc(-c2cn(C)c(=O)c3cnccc23)cc(OC)c1CN(C)C(=O)CCCCN1CCC2(CCN(c3ccc4c(c3)C(=O)N(C3CCC(=O)NC3=O)C4=O)C2)C1] |
| D189 | 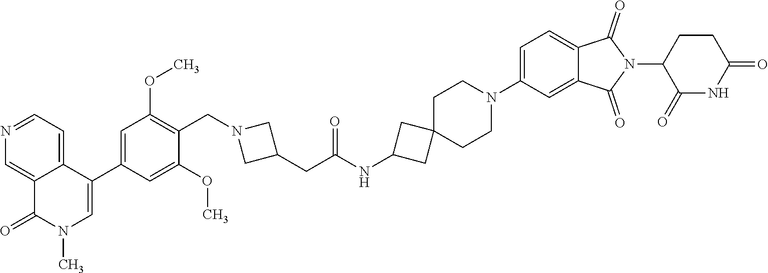 [SMILES: COc1cc(-c2cn(C)c(=O)c3cnccc23)cc(OC)c1CN1CC(CC(=O)NC2CC3(CCN(c4ccc5c(c4)C(=O)N(C4CCC(=O)NC4=O)C5=O)CC3)C2)C1] |
| D190 | 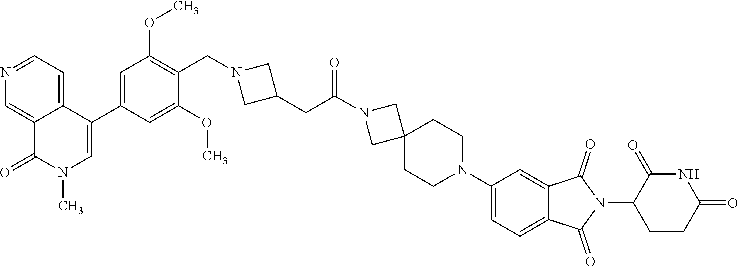 [SMILES: COc1cc(-c2cn(C)c(=O)c3cnccc23)cc(OC)c1CN1CC(CC(=O)N2CC3(CCN(c4ccc5c(c4)C(=O)N(C4CCC(=O)NC4=O)C5=O)CC3)C2)C1] |
| D191 | 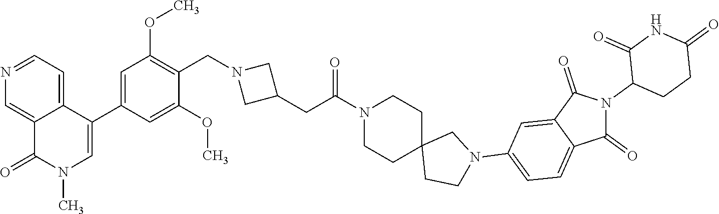 [SMILES: COc1cc(-c2cn(C)c(=O)c3cnccc23)cc(OC)c1CN1CC(CC(=O)N2CCC3(CC2)CCN(c2ccc4c(c2)C(=O)N(C2CCC(=O)NC2=O)C4=O)C3)C1] |
| D192 | 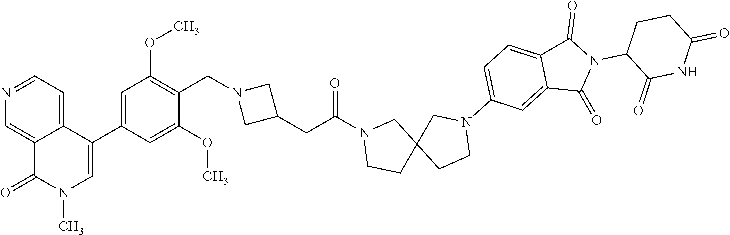 [SMILES: COc1cc(-c2cn(C)c(=O)c3cnccc23)cc(OC)c1CN1CC(CC(=O)N2CCC3(CCN(c4ccc5c(c4)C(=O)N(C4CCC(=O)NC4=O)C5=O)C3)C2)C1] |
| D193 | 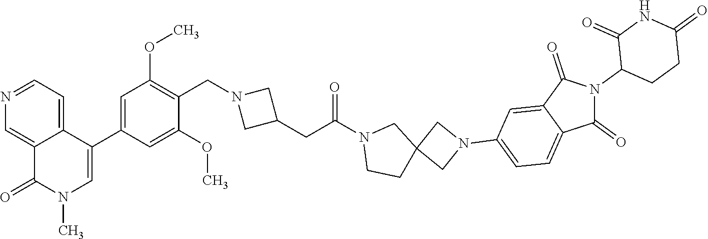 [SMILES: COc1cc(-c2cn(C)c(=O)c3cnccc23)cc(OC)c1CN1CC(CC(=O)N2CCC3(C2)CN(c2ccc4c(c2)C(=O)N(C2CCC(=O)NC2=O)C4=O)C3)C1] |
| D194 | 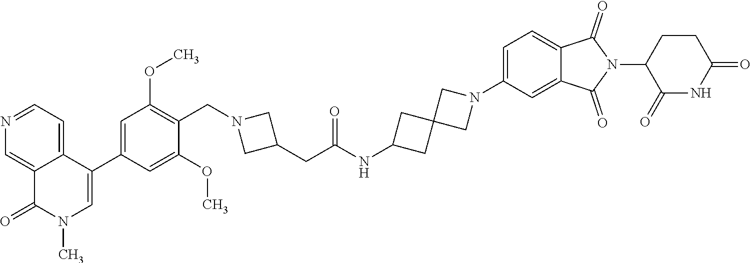 [SMILES: COc1cc(-c2cn(C)c(=O)c3cnccc23)cc(OC)c1CN1CC(CC(=O)NC2CC3(C2)CN(c2ccc4c(c2)C(=O)N(C2CCC(=O)NC2=O)C4=O)C3)C1] |
| D195 | 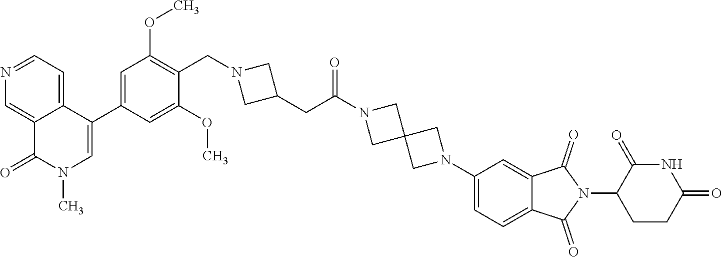 [SMILES: COc1cc(-c2cn(C)c(=O)c3cnccc23)cc(OC)c1CN1CC(CC(=O)N2CC3(C2)CN(c2ccc4c(c2)C(=O)N(C2CCC(=O)NC2=O)C4=O)C3)C1] |
| D196 | 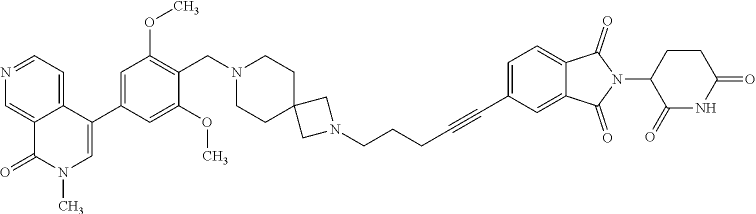 [SMILES: COc1cc(-c2cn(C)c(=O)c3cnccc23)cc(OC)c1CN1CCC2(CC1)CN(CCCC#Cc1ccc3c(c1)C(=O)N(C1CCC(=O)NC1=O)C3=O)C2] |
| D197 | 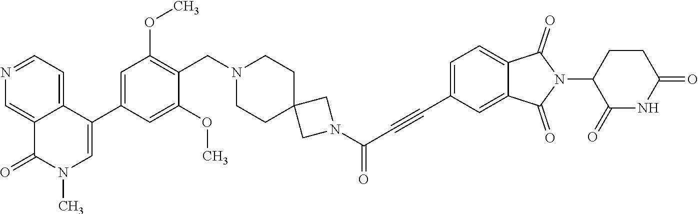 [SMILES: COc1cc(-c2cn(C)c(=O)c3cnccc23)cc(OC)c1CN1CCC2(CC1)CN(C(=O)C#Cc1ccc3c(c1)C(=O)N(C1CCC(=O)NC1=O)C3=O)C2] |
| D198 | 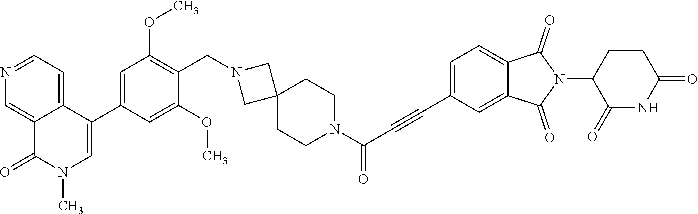 [SMILES: COc1cc(-c2cn(C)c(=O)c3cnccc23)cc(OC)c1CN1CC2(CCN(C(=O)C#Cc3ccc4c(c3)C(=O)N(C3CCC(=O)NC3=O)C4=O)CC2)C1] |
| D199 | 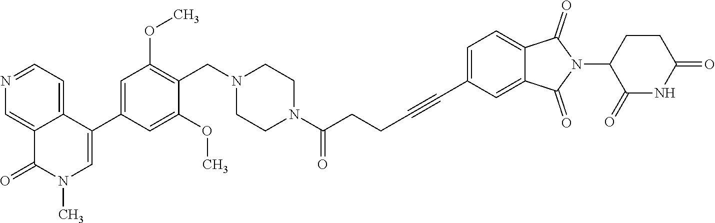 [SMILES: COc1cc(-c2cn(C)c(=O)c3cnccc23)cc(OC)c1CN1CCN(C(=O)CCC#Cc2ccc3c(c2)C(=O)N(C2CCC(=O)NC2=O)C3=O)CC1] |
| D200 | 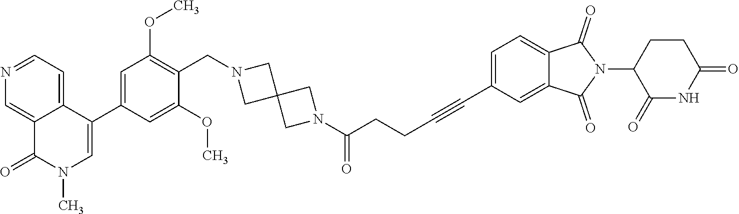 [SMILES: COc1cc(-c2cn(C)c(=O)c3cnccc23)cc(OC)c1CN1CC2(C1)CN(C(=O)CCC#Cc1ccc3c(c1)C(=O)N(C1CCC(=O)NC1=O)C3=O)C2] |
| D201 | 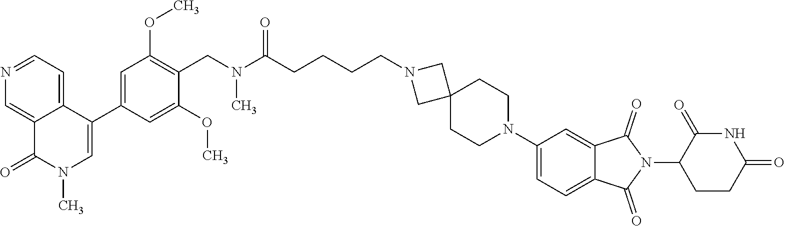 [SMILES: COc1cc(-c2cn(C)c(=O)c3cnccc23)cc(OC)c1CN(C)C(=O)CCCCN1CC2(CCN(c3ccc4c(c3)C(=O)N(C3CCC(=O)NC3=O)C4=O)CC2)C1] |
| D202 | 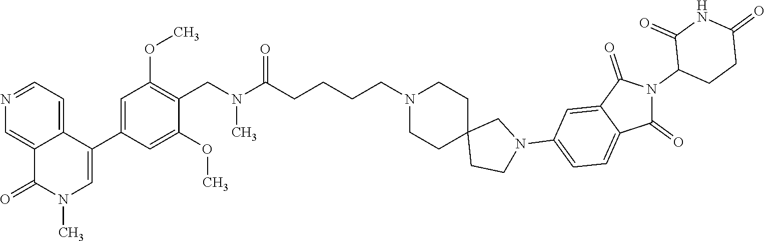 [SMILES: COc1cc(-c2cn(C)c(=O)c3cnccc23)cc(OC)c1CN(C)C(=O)CCCCN1CCC2(CC1)CCN(c1ccc3c(c1)C(=O)N(C1CCC(=O)NC1=O)C3=O)C2] |
| D203 | 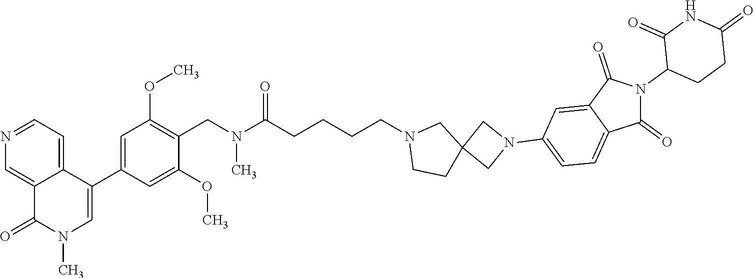 [SMILES: COc1cc(-c2cn(C)c(=O)c3cnccc23)cc(OC)c1CN(C)C(=O)CCCCN1CCC2(C1)CN(c1ccc3c(c1)C(=O)N(C1CCC(=O)NC1=O)C3=O)C2] |
| D204 | 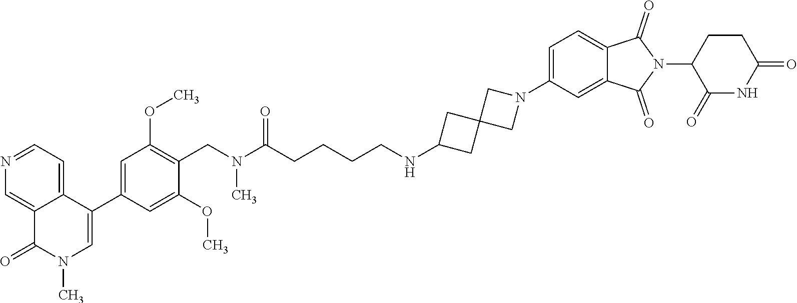 [SMILES: COc1cc(-c2cn(C)c(=O)c3cnccc23)cc(OC)c1CN(C)C(=O)CCCCNC1CC2(C1)CN(c1ccc3c(c1)C(=O)N(C1CCC(=O)NC1=O)C3=O)C2] |
| D205 | 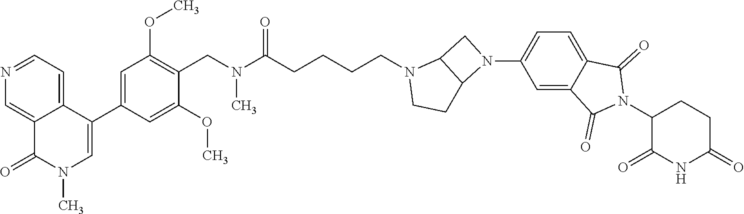 [SMILES: COc1cc(-c2cn(C)c(=O)c3cnccc23)cc(OC)c1CN(C)C(=O)CCCCN1CCC2C1CN2c1ccc2c(c1)C(=O)N(C1CCC(=O)NC1=O)C2=O] |
| D206 | 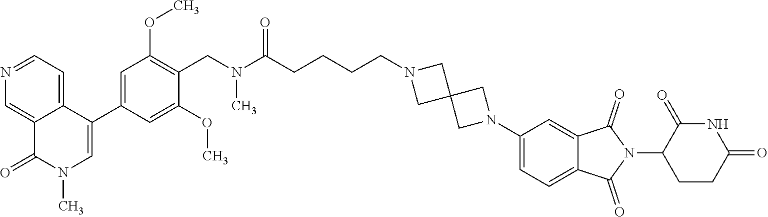 [SMILES: COc1cc(-c2cn(C)c(=O)c3cnccc23)cc(OC)c1CN(C)C(=O)CCCCN1CC2(C1)CN(c1ccc3c(c1)C(=O)N(C1CCC(=O)NC1=O)C3=O)C2] |
| D207 | 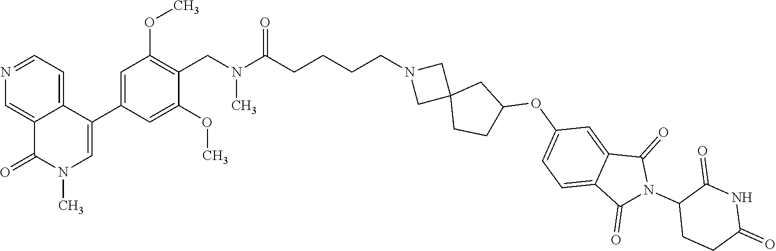 [SMILES: COc1cc(-c2cn(C)c(=O)c3cnccc23)cc(OC)c1CN(C)C(=O)CCCCN1CC2(CCC(Oc3ccc4c(c3)C(=O)N(C3CCC(=O)NC3=O)C4=O)C2)C1] |
| D208 | 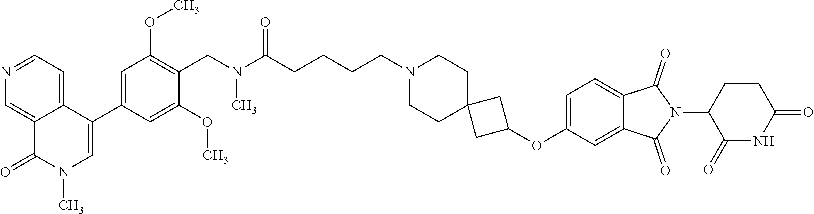 [SMILES: COc1cc(-c2cn(C)c(=O)c3cnccc23)cc(OC)c1CN(C)C(=O)CCCCN1CCC2(CC1)CC(Oc1ccc3c(c1)C(=O)N(C1CCC(=O)NC1=O)C3=O)C2] |
| D209 | 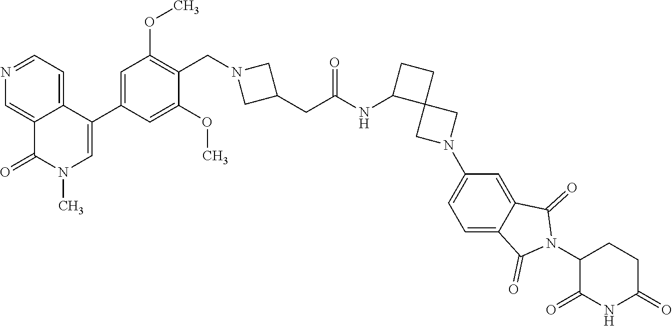 [SMILES: COc1cc(-c2cn(C)c(=O)c3cnccc23)cc(OC)c1CN1CC(CC(=O)NC2CCC23CN(c2ccc4c(c2)C(=O)N(C2CCC(=O)NC2=O)C4=O)C3)C1] |
| D210 | 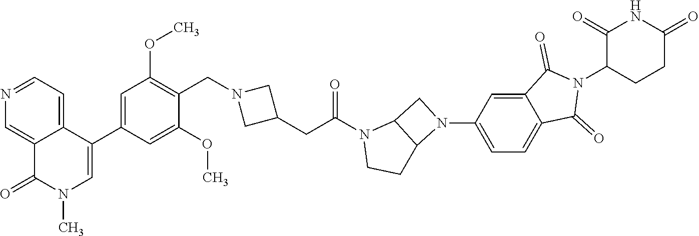 [SMILES: COc1cc(-c2cn(C)c(=O)c3cnccc23)cc(OC)c1CN1CC(CC(=O)N2CCC3C2CN3c2ccc3c(c2)C(=O)N(C2CCC(=O)NC2=O)C3=O)C1] |
| D211 | 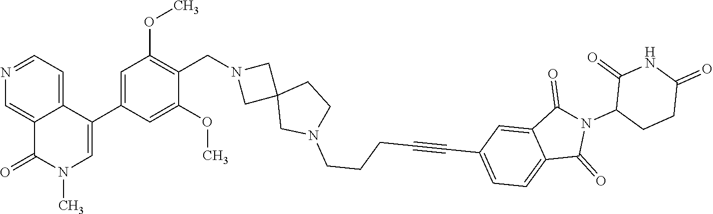 [SMILES: COc1cc(-c2cn(C)c(=O)c3cnccc23)cc(OC)c1CN1CC2(CCN(CCCC#Cc3ccc4c(c3)C(=O)N(C3CCC(=O)NC3=O)C4=O)C2)C1] |
| D212 | 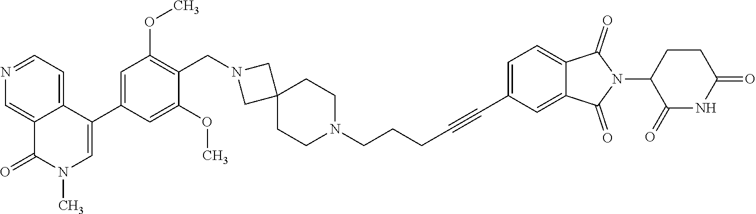 [SMILES: COc1cc(-c2cn(C)c(=O)c3cnccc23)cc(OC)c1CN1CC2(CCN(CCCC#Cc3ccc4c(c3)C(=O)N(C3CCC(=O)NC3=O)C4=O)CC2)C1] |
| D213 | 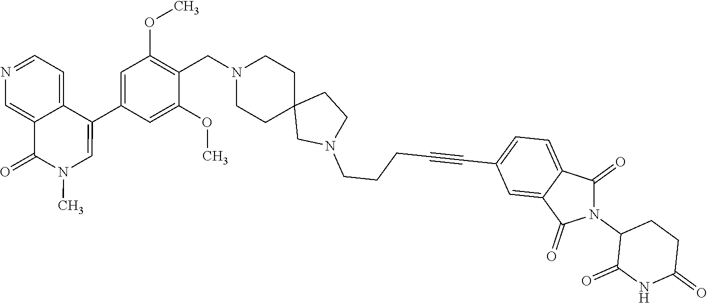 [SMILES: COc1cc(-c2cn(C)c(=O)c3cnccc23)cc(OC)c1CN1CCC2(CC1)CCN(CCCC#Cc1ccc3c(c1)C(=O)N(C1CCC(=O)NC1=O)C3=O)C2] |
| D214 | 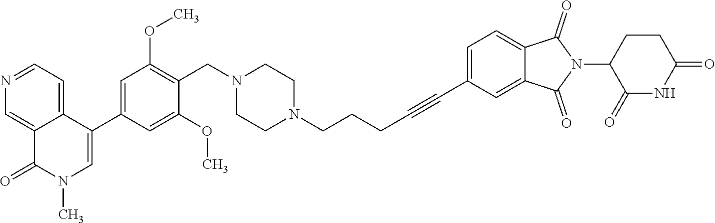 [SMILES: COc1cc(-c2cn(C)c(=O)c3cnccc23)cc(OC)c1CN1CCN(CCCC#Cc2ccc3c(c2)C(=O)N(C2CCC(=O)NC2=O)C3=O)CC1] |
| D215 | 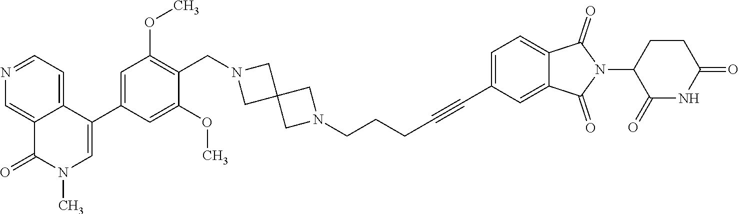 [SMILES: COc1cc(-c2cn(C)c(=O)c3cnccc23)cc(OC)c1CN1CC2(CN(CCCC#Cc3ccc4c(c3)C(=O)N(C3CCC(=O)NC3=O)C4=O)C2)C1] |
| D216 | 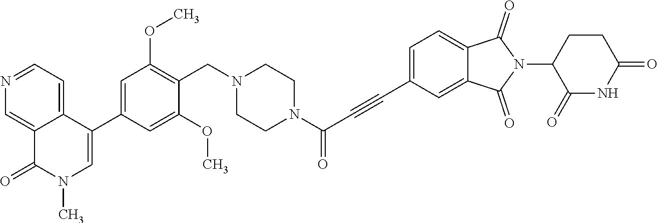 [SMILES: COc1cc(-c2cn(C)c(=O)c3cnccc23)cc(OC)c1CN1CCN(C(=O)C#Cc2ccc3c(c2)C(=O)N(C2CCC(=O)NC2=O)C3=O)CC1] |
| D217 | 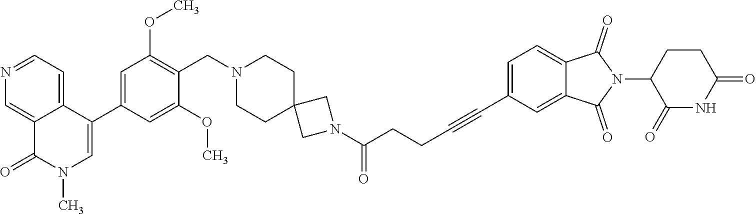 [SMILES: COc1cc(-c2cn(C)c(=O)c3cnccc23)cc(OC)c1CN1CCC2(CC1)CN(C(=O)CCC#Cc1ccc3c(c1)C(=O)N(C1CCC(=O)NC1=O)C3=O)C2] |
| D218 | 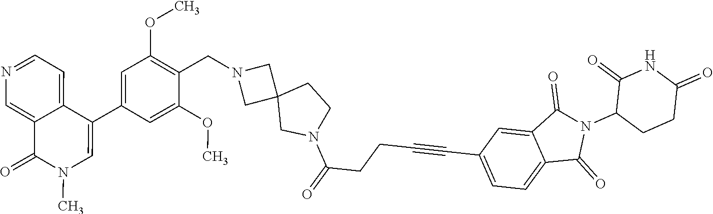 [SMILES: COc1cc(-c2cn(C)c(=O)c3cnccc23)cc(OC)c1CN1CC2(CCN(C(=O)CCC#Cc3ccc4c(c3)C(=O)N(C3CCC(=O)NC3=O)C4=O)C2)C1] |
| D219 | 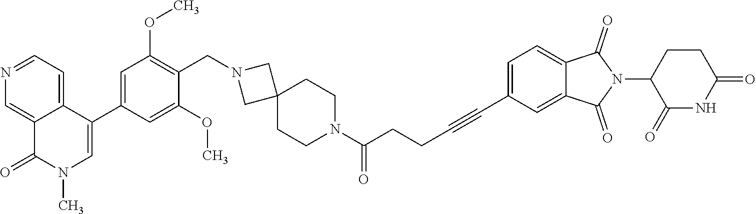 [SMILES: COc1cc(-c2cn(C)c(=O)c3cnccc23)cc(OC)c1CN1CC2(CCN(C(=O)CCC#Cc3ccc4c(c3)C(=O)N(C3CCC(=O)NC3=O)C4=O)CC2)C1] |
| D220 | 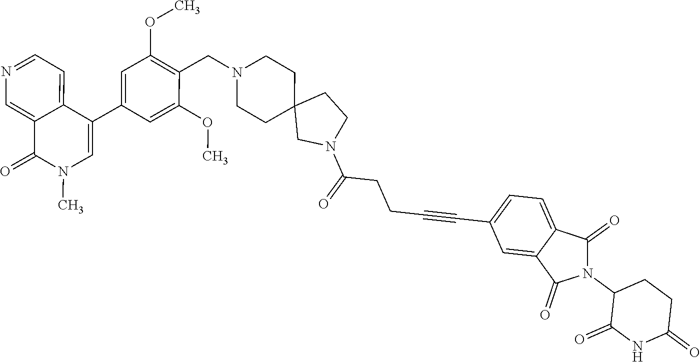 [SMILES: COc1cc(-c2cn(C)c(=O)c3cnccc23)cc(OC)c1CN1CCC2(CC1)CCN(C(=O)CCC#Cc1ccc3c(c1)C(=O)N(C1CCC(=O)NC1=O)C3=O)C2] |
| D221 | 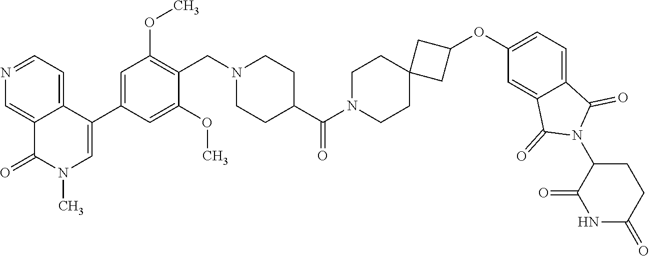 [SMILES: COc1cc(-c2cn(C)c(=O)c3cnccc23)cc(OC)c1CN1CCC(C(=O)N2CCC3(CC2)CC(Oc2ccc4c(c2)C(=O)N(C2CCC(=O)NC2=O)C4=O)C3)CC1] |
| D222 | 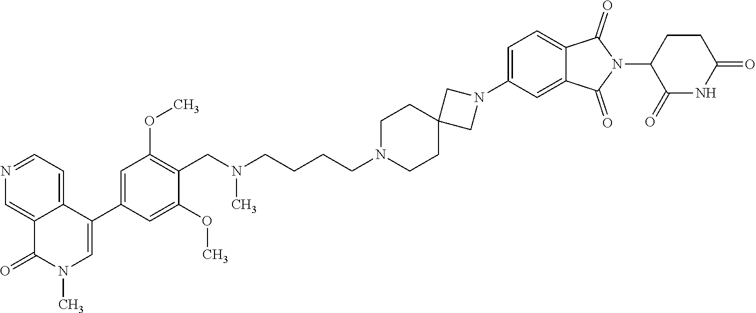 [SMILES: COc1cc(-c2cn(C)c(=O)c3cnccc23)cc(OC)c1CN(C)CCCCN1CCC2(CC1)CN(c1ccc3c(c1)C(=O)N(C1CCC(=O)NC1=O)C3=O)C2] |
| D223 | 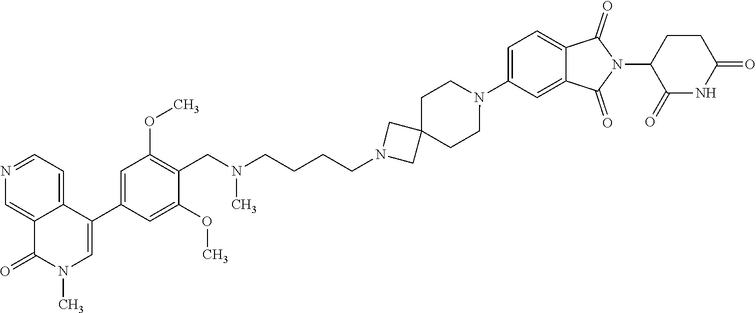 [SMILES: COc1cc(-c2cn(C)c(=O)c3cnccc23)cc(OC)c1CN(C)CCCCN1CC2(CCN(c3ccc4c(c3)C(=O)N(C3CCC(=O)NC3=O)C4=O)CC2)C1] |
| D224 | 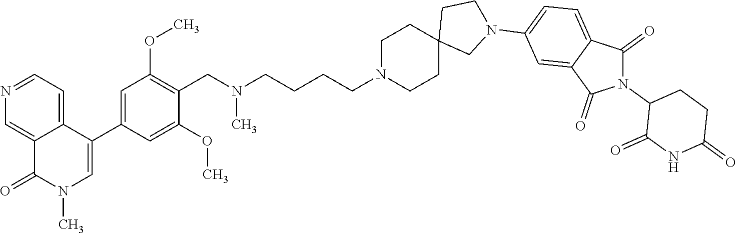 [SMILES: COc1cc(-c2cn(C)c(=O)c3cnccc23)cc(OC)c1CN(C)CCCCN1CCC2(CC1)CCN(c1ccc3c(c1)C(=O)N(C1CCC(=O)NC1=O)C3=O)C2] |
| D225 | 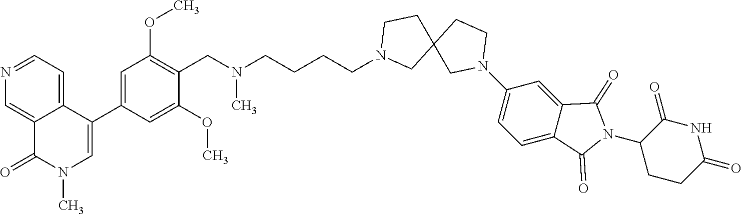 [SMILES: COc1cc(-c2cn(C)c(=O)c3cnccc23)cc(OC)c1CN(C)CCCCN1CCC2(CCN(c3ccc4c(c3)C(=O)N(C3CCC(=O)NC3=O)C4=O)C2)C1] |
| D226 | 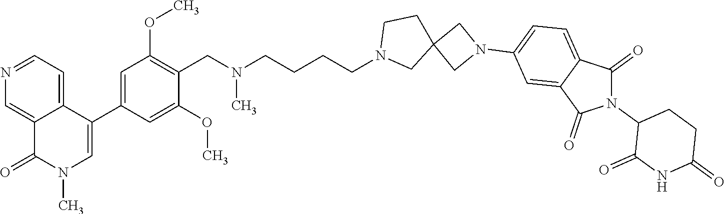 [SMILES: COc1cc(-c2cn(C)c(=O)c3cnccc23)cc(OC)c1CN(C)CCCCN1CCC2(C1)CN(c1ccc3c(c1)C(=O)N(C1CCC(=O)NC1=O)C3=O)C2] |
| D227 | 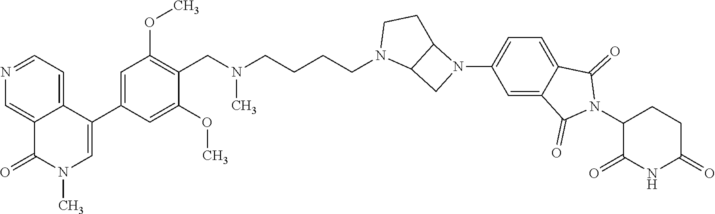 [SMILES: COc1cc(-c2cn(C)c(=O)c3cnccc23)cc(OC)c1CN(C)CCCCN1CCC2C1CN2c1ccc2c(c1)C(=O)N(C1CCC(=O)NC1=O)C2=O] |
| D228 | 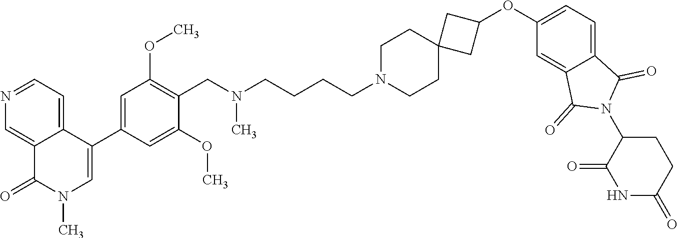 [SMILES: COc1cc(-c2cn(C)c(=O)c3cnccc23)cc(OC)c1CN(C)CCCCN1CCC2(CC1)CC(Oc1ccc3c(c1)C(=O)N(C1CCC(=O)NC1=O)C3=O)C2] |
| D229 | 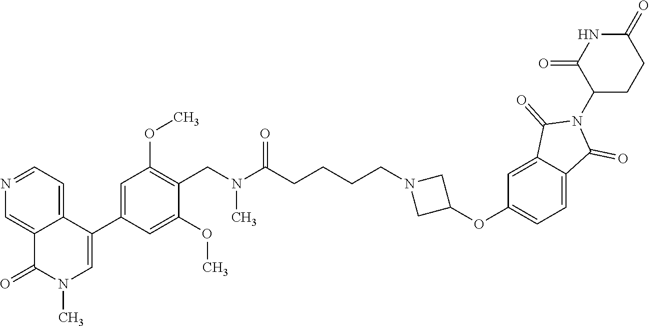 [SMILES: COc1cc(-c2cn(C)c(=O)c3cnccc23)cc(OC)c1CN(C)C(=O)CCCCN1CC(Oc2ccc3c(c2)C(=O)N(C2CCC(=O)NC2=O)C3=O)C1] |
| D230 | 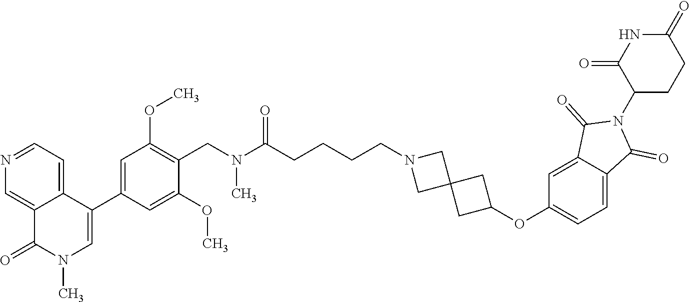 [SMILES: COc1cc(-c2cn(C)c(=O)c3cnccc23)cc(OC)c1CN(C)C(=O)CCCCN1CC2(CC(Oc3ccc4c(c3)C(=O)N(C3CCC(=O)NC3=O)C4=O)C2)C1] |
| D231 | 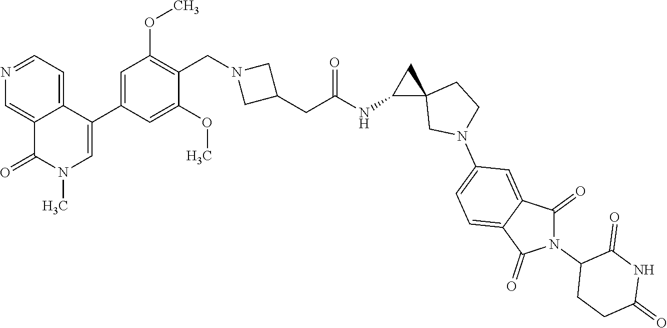 [SMILES: COc1cc(-c2cn(C)c(=O)c3cnccc23)cc(OC)c1CN1CC(CC(=O)N[C@@H]2C[C@]23CCN(c2ccc4c(c2)C(=O)N(C2CCC(=O)NC2=O)C4=O)C3)C1] |
| D232 | 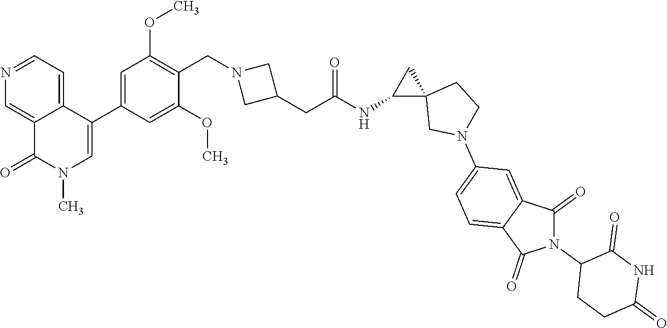 [SMILES: COc1cc(-c2cn(C)c(=O)c3cnccc23)cc(OC)c1CN1CC(CC(=O)N[C@@H]2C[C@@]23CCN(c2ccc4c(c2)C(=O)N(C2CCC(=O)NC2=O)C4=O)C3)C1] |
| D233 | 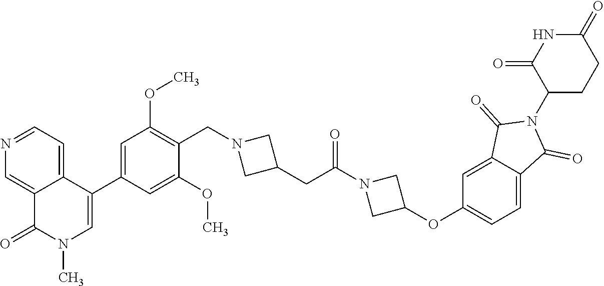 [SMILES: COc1cc(-c2cn(C)c(=O)c3cnccc23)cc(OC)c1CN1CC(CC(=O)N2CC(Oc3ccc4c(c3)C(=O)N(C3CCC(=O)NC3=O)C4=O)C2)C1] |
| D234 | 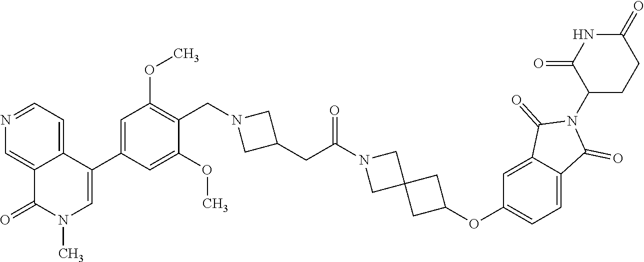 [SMILES: COc1cc(-c2cn(C)c(=O)c3cnccc23)cc(OC)c1CN1CC(CC(=O)N2CC3(CC(Oc4ccc5c(c4)C(=O)N(C4CCC(=O)NC4=O)C5=O)C3)C2)C1] |
| D235 | 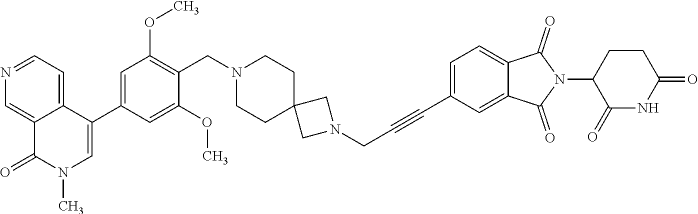 [SMILES: COc1cc(-c2cn(C)c(=O)c3cnccc23)cc(OC)c1CN1CCC2(CC1)CN(CC#Cc1ccc3c(c1)C(=O)N(C1CCC(=O)NC1=O)C3=O)C2] |
| D236 | 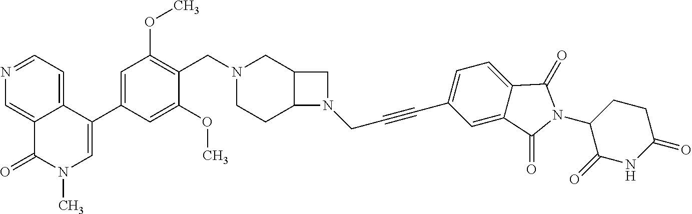 [SMILES: COc1cc(-c2cn(C)c(=O)c3cnccc23)cc(OC)c1CN1CCC2C(C1)CN2CC#Cc1ccc2c(c1)C(=O)N(C1CCC(=O)NC1=O)C2=O] |
| D237 | 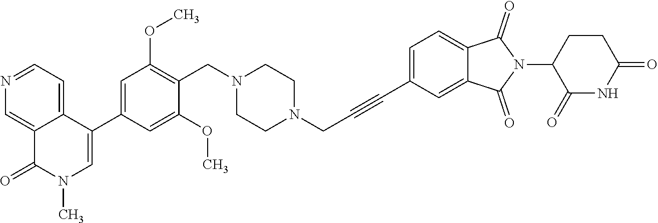 [SMILES: COc1cc(-c2cn(C)c(=O)c3cnccc23)cc(OC)c1CN1CCN(CC#Cc2ccc3c(c2)C(=O)N(C2CCC(=O)NC2=O)C3=O)CC1] |
| D238 | 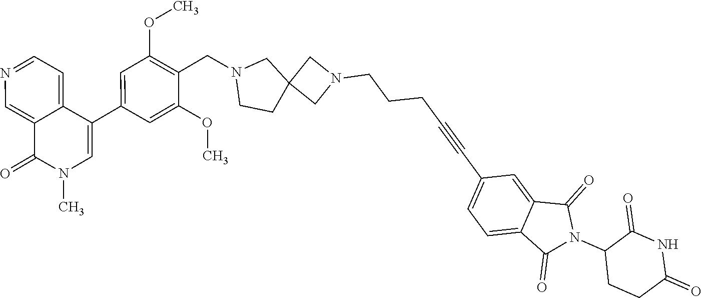 [SMILES: COc1cc(-c2cn(C)c(=O)c3cnccc23)cc(OC)c1CN1CCC2(C1)CN(CCCC#Cc1ccc3c(c1)C(=O)N(C1CCC(=O)NC1=O)C3=O)C2] |
| D239 | 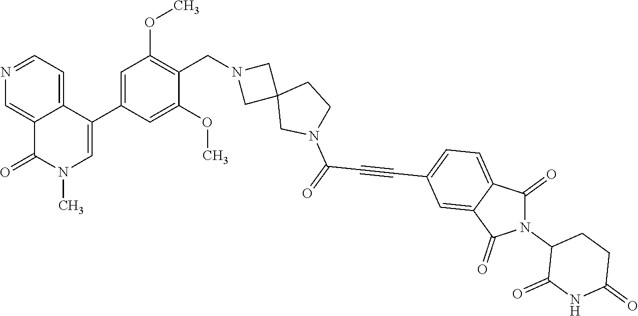 [SMILES: COc1cc(-c2cn(C)c(=O)c3cnccc23)cc(OC)c1CN1CC2(CCN(C(=O)C#Cc3ccc4c(c3)C(=O)N(C3CCC(=O)NC3=O)C4=O)C2)C1] |
| D240 | 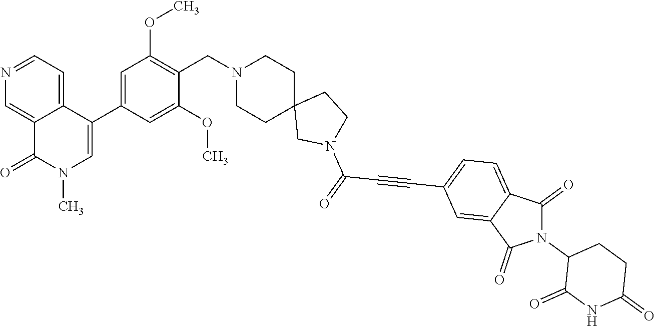 [SMILES: COc1cc(-c2cn(C)c(=O)c3cnccc23)cc(OC)c1CN1CCC2(CC1)CCN(C(=O)C#Cc1ccc3c(c1)C(=O)N(C1CCC(=O)NC1=O)C3=O)C2] |
| D241 | 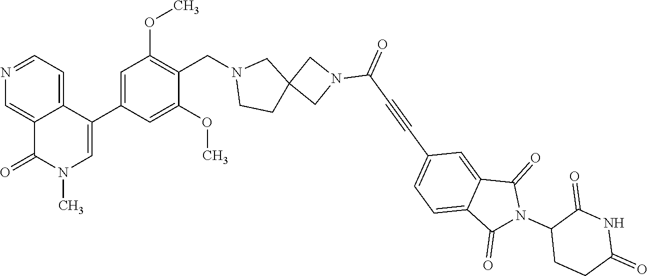 [SMILES: COc1cc(-c2cn(C)c(=O)c3cnccc23)cc(OC)c1CN1CCC2(C1)CN(C(=O)C#Cc1ccc3c(c1)C(=O)N(C1CCC(=O)NC1=O)C3=O)C2] |
| D242 | 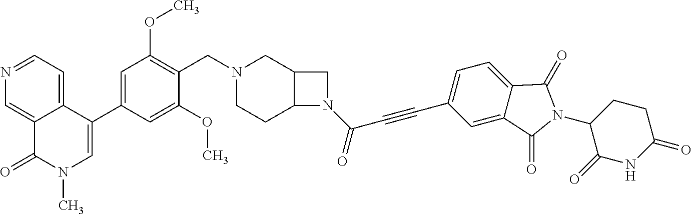 [SMILES: COc1cc(-c2cn(C)c(=O)c3cnccc23)cc(OC)c1CN1CCC2C(C1)CN2C(=O)C#Cc1ccc2c(c1)C(=O)N(C1CCC(=O)NC1=O)C2=O] |
| D243 | 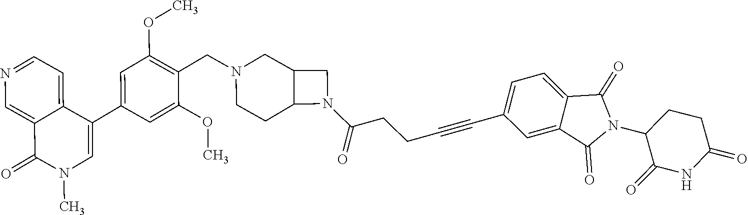 [SMILES: COc1cc(-c2cn(C)c(=O)c3cnccc23)cc(OC)c1CN1CCC2C(C1)CN2C(=O)CCC#Cc1ccc2c(c1)C(=O)N(C1CCC(=O)NC1=O)C2=O] |
| D244 | 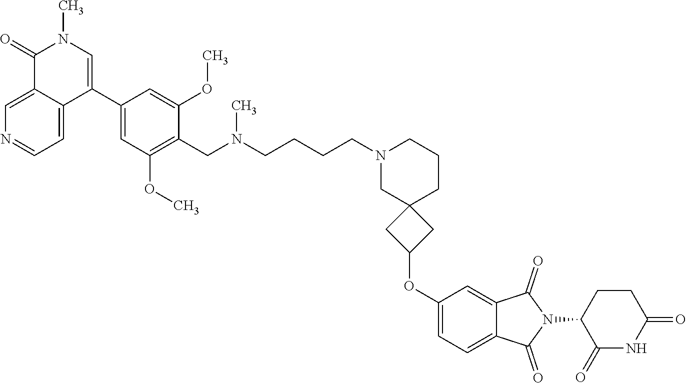 [SMILES: COc1cc(-c2cn(C)c(=O)c3cnccc23)cc(OC)c1CN(C)CCCCN1CCCC2(CC(Oc3ccc4c(c3)C(=O)N([C@@H]3CCC(=O)NC3=O)C4=O)C2)C1] |
| D245 | 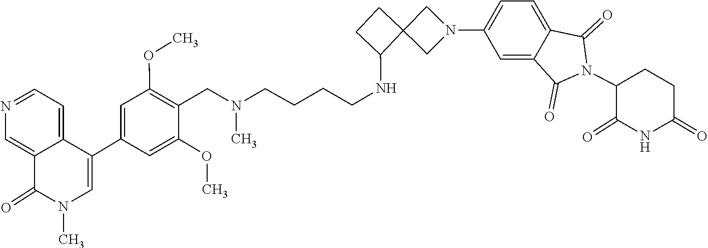 [SMILES: COc1cc(-c2cn(C)c(=O)c3cnccc23)cc(OC)c1CN(C)CCCCNC1CCC12CN(c1ccc3c(c1)C(=O)N(C1CCC(=O)NC1=O)C3=O)C2] |
| D246 | 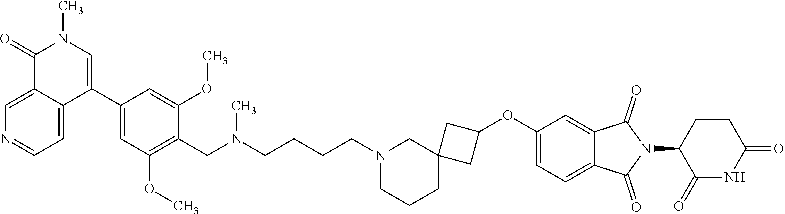 [SMILES: COc1cc(-c2cn(C)c(=O)c3cnccc23)cc(OC)c1CN(C)CCCCN1CCCC2(CC(Oc3ccc4c(c3)C(=O)N([C@H]3CCC(=O)NC3=O)C4=O)C2)C1] |
| D247 | 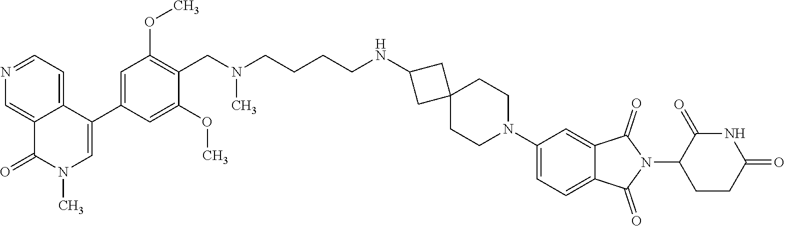 [SMILES: COc1cc(-c2cn(C)c(=O)c3cnccc23)cc(OC)c1CN(C)CCCCNC1CC2(CCN(c3ccc4c(c3)C(=O)N(C3CCC(=O)NC3=O)C4=O)CC2)C1] |
| D248 | 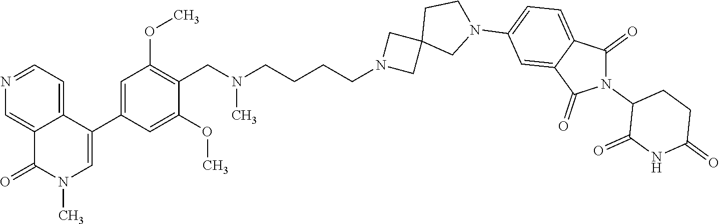 [SMILES: COc1cc(-c2cn(C)c(=O)c3cnccc23)cc(OC)c1CN(C)CCCCN1CC2(CCN(c3ccc4c(c3)C(=O)N(C3CCC(=O)NC3=O)C4=O)C2)C1] |
| D249 | 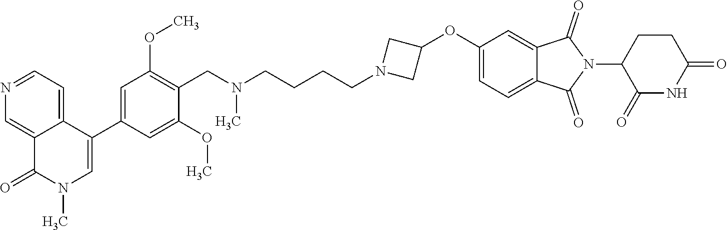 [SMILES: COc1cc(-c2cn(C)c(=O)c3cnccc23)cc(OC)c1CN(C)CCCCN1CC(Oc2ccc3c(c2)C(=O)N(C2CCC(=O)NC2=O)C3=O)C1] |
| D250 | 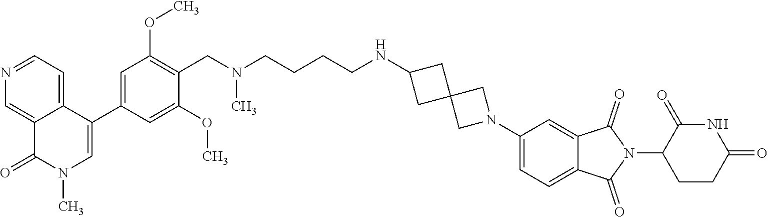 [SMILES: COc1cc(-c2cn(C)c(=O)c3cnccc23)cc(OC)c1CN(C)CCCCNC1CC2(C1)CN(c1ccc3c(c1)C(=O)N(C1CCC(=O)NC1=O)C3=O)C2] |
| D251 | 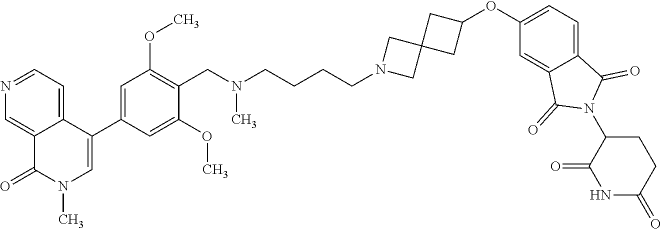 [SMILES: COc1cc(-c2cn(C)c(=O)c3cnccc23)cc(OC)c1CN(C)CCCCN1CC2(CC(Oc3ccc4c(c3)C(=O)N(C3CCC(=O)NC3=O)C4=O)C2)C1] |
| D252 | 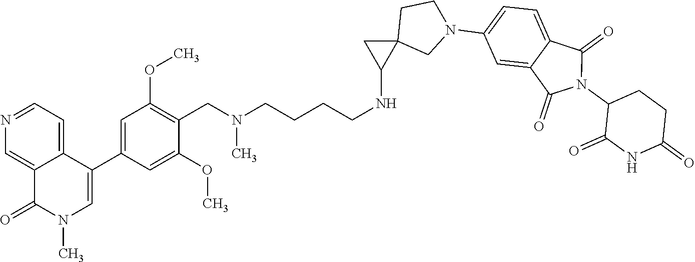 [SMILES: COc1cc(-c2cn(C)c(=O)c3cnccc23)cc(OC)c1CN(C)CCCCNC1CC12CCN(c1ccc3c(c1)C(=O)N(C1CCC(=O)NC1=O)C3=O)C2] |
| D253 | 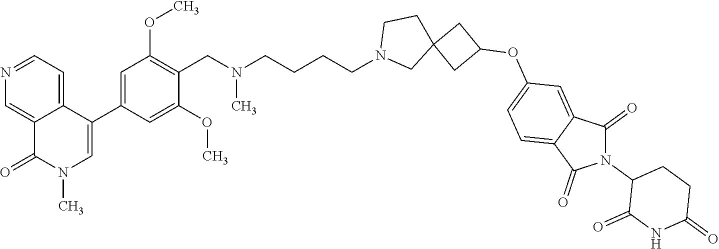 [SMILES: COc1cc(-c2cn(C)c(=O)c3cnccc23)cc(OC)c1CN(C)CCCCN1CCC2(CC(Oc3ccc4c(c3)C(=O)N(C3CCC(=O)NC3=O)C4=O)C2)C1] |
| D254 | 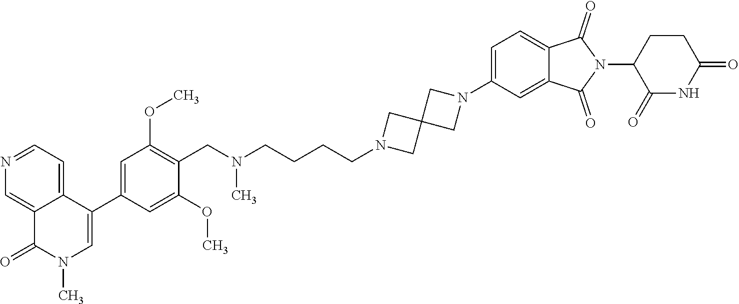 [SMILES: COc1cc(-c2cn(C)c(=O)c3cnccc23)cc(OC)c1CN(C)CCCCN1CC2(C1)CN(c1ccc3c(c1)C(=O)N(C1CCC(=O)NC1=O)C3=O)C2] |
| D255 | 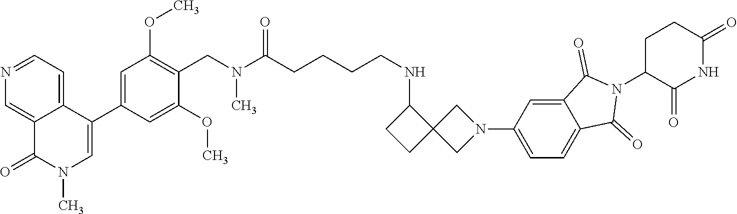 [SMILES: COc1cc(-c2cn(C)c(=O)c3cnccc23)cc(OC)c1CN(C)C(=O)CCCCNC1CCC12CN(c1ccc3c(c1)C(=O)N(C1CCC(=O)NC1=O)C3=O)C2] |
| D256 | 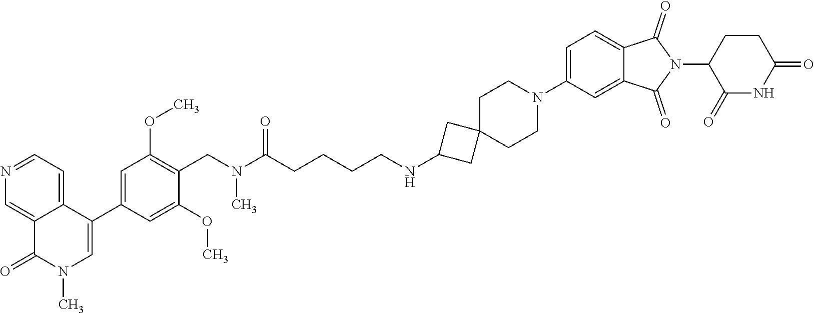 [SMILES: COc1cc(-c2cn(C)c(=O)c3cnccc23)cc(OC)c1CN(C)C(=O)CCCCNC1CC2(CCN(c3ccc4c(c3)C(=O)N(C3CCC(=O)NC3=O)C4=O)CC2)C1] |
| D257 | 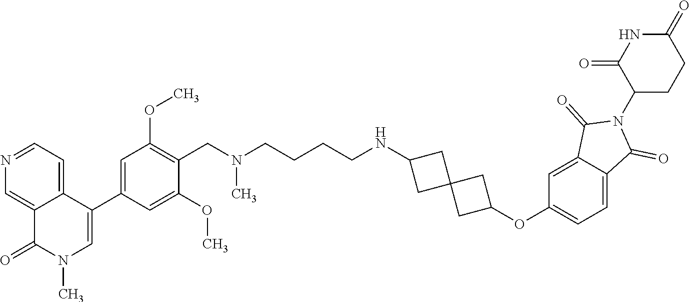 [SMILES: COc1cc(-c2cn(C)c(=O)c3cnccc23)cc(OC)c1CN(C)CCCCNC1CC2(C1)CC(Oc1ccc3c(c1)C(=O)N(C1CCC(=O)NC1=O)C3=O)C2] |
| D258 | 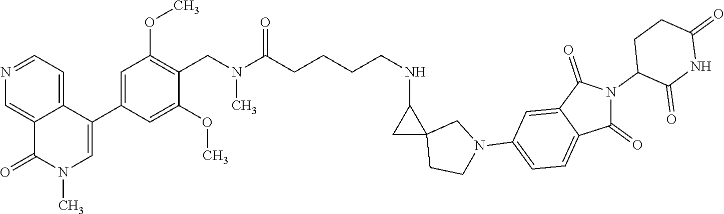 [SMILES: COc1cc(-c2cn(C)c(=O)c3cnccc23)cc(OC)c1CN(C)C(=O)CCCCNC1CC12CCN(c1ccc3c(c1)C(=O)N(C1CCC(=O)NC1=O)C3=O)C2] |
| D259 | 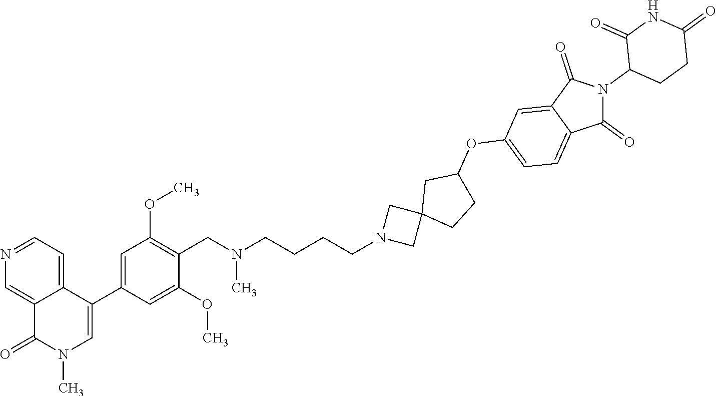 [SMILES: COc1cc(-c2cn(C)c(=O)c3cnccc23)cc(OC)c1CN(C)CCCCN1CC2(CCC(Oc3ccc4c(c3)C(=O)N(C3CCC(=O)NC3=O)C4=O)C2)C1] |
| D260 | 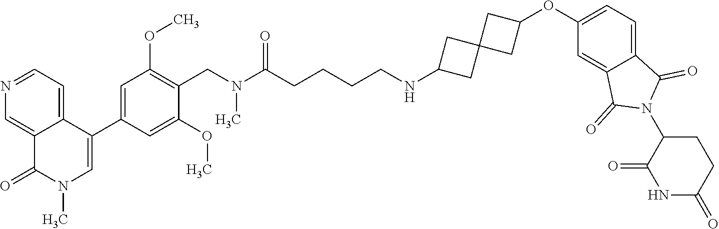 [SMILES: COc1cc(-c2cn(C)c(=O)c3cnccc23)cc(OC)c1CN(C)C(=O)CCCCNC1CC2(C1)CC(Oc1ccc3c(c1)C(=O)N(C1CCC(=O)NC1=O)C3=O)C2] |
| D261 | 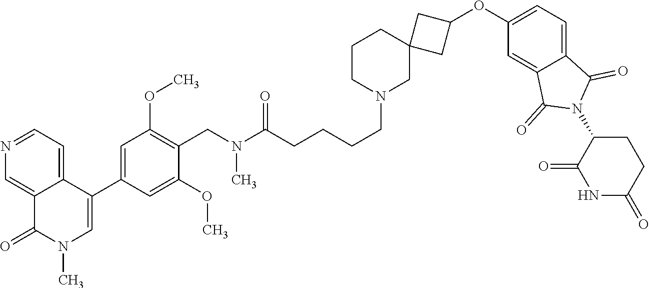 [SMILES: COc1cc(-c2cn(C)c(=O)c3cnccc23)cc(OC)c1CN(C)C(=O)CCCCN1CCCC2(CC(Oc3ccc4c(c3)C(=O)N([C@@H]3CCC(=O)NC3=O)C4=O)C2)C1] |
| D262 | 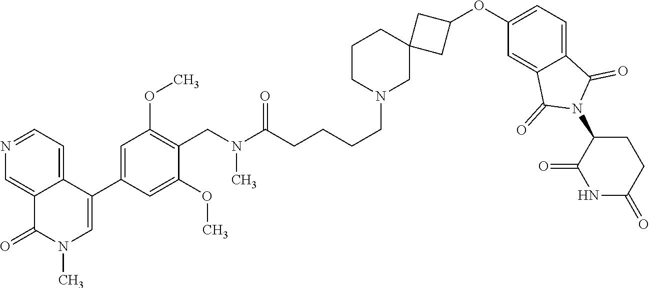 [SMILES: COc1cc(-c2cn(C)c(=O)c3cnccc23)cc(OC)c1CN(C)C(=O)CCCCN1CCCC2(CC(Oc3ccc4c(c3)C(=O)N([C@H]3CCC(=O)NC3=O)C4=O)C2)C1] |
| D263 | 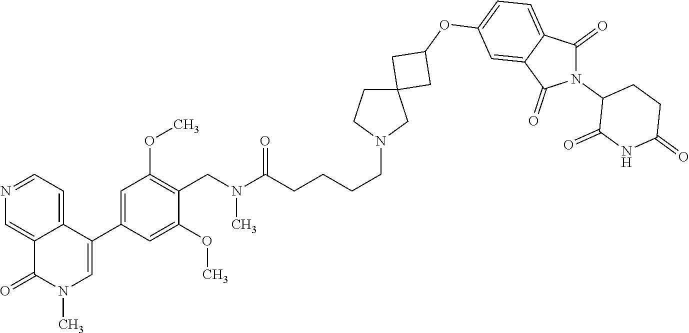 [SMILES: COc1cc(-c2cn(C)c(=O)c3cnccc23)cc(OC)c1CN(C)C(=O)CCCCN1CCC2(CC(Oc3ccc4c(c3)C(=O)N(C3CCC(=O)NC3=O)C4=O)C2)C1] |
| D264 | 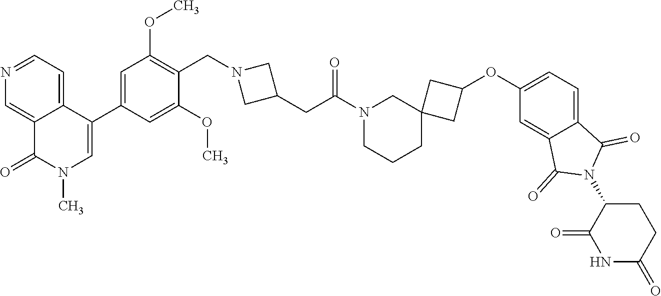 [SMILES: COc1cc(-c2cn(C)c(=O)c3cnccc23)cc(OC)c1CN1CC(CC(=O)N2CCCC3(CC(Oc4ccc5c(c4)C(=O)N([C@@H]4CCC(=O)NC4=O)C5=O)C3)C2)C1] |
| D265 | 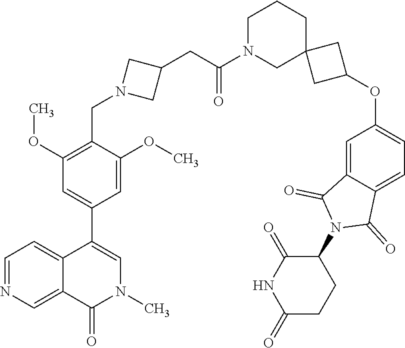 [SMILES: COc1cc(-c2cn(C)c(=O)c3cnccc23)cc(OC)c1CN1CC(CC(=O)N2CCCC3(CC(Oc4ccc5c(c4)C(=O)N([C@H]4CCC(=O)NC4=O)C5=O)C3)C2)C1] |
| D266 | 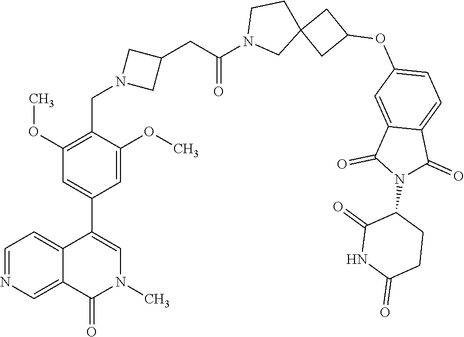 [SMILES: COc1cc(-c2cn(C)c(=O)c3cnccc23)cc(OC)c1CN1CC(CC(=O)N2CCC3(CC(Oc4ccc5c(c4)C(=O)N([C@@H]4CCC(=O)NC4=O)C5=O)C3)C2)C1] |
| D267 | 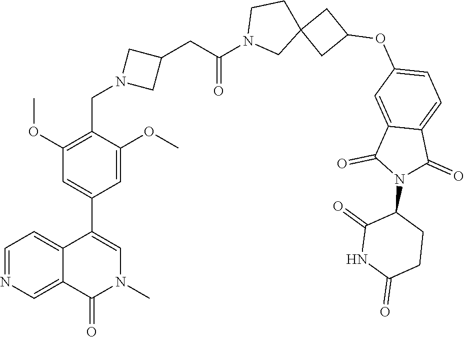 [SMILES: COc1cc(-c2cn(C)c(=O)c3cnccc23)cc(OC)c1CN1CC(CC(=O)N2CCC3(CC(Oc4ccc5c(c4)C(=O)N([C@H]4CCC(=O)NC4=O)C5=O)C3)C2)C1] |
| D268 | 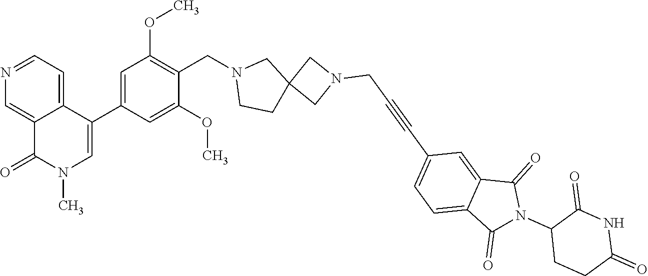 [SMILES: COc1cc(-c2cn(C)c(=O)c3cnccc23)cc(OC)c1CN1CCC2(C1)CN(CC#Cc1ccc3c(c1)C(=O)N(C1CCC(=O)NC1=O)C3=O)C2] |
| D269 | 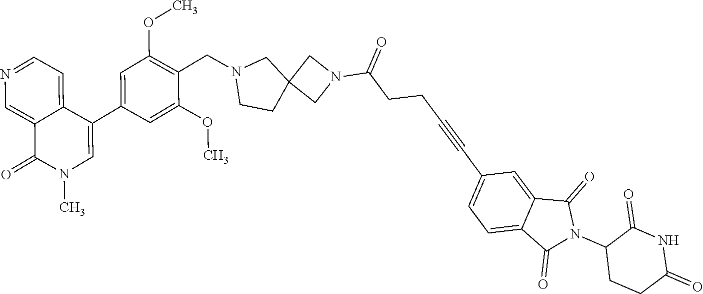 [SMILES: COc1cc(-c2cn(C)c(=O)c3cnccc23)cc(OC)c1CN1CCC2(C1)CN(C(=O)CCC#Cc1ccc3c(c1)C(=O)N(C1CCC(=O)NC1=O)C3=O)C2] |
| D270 | 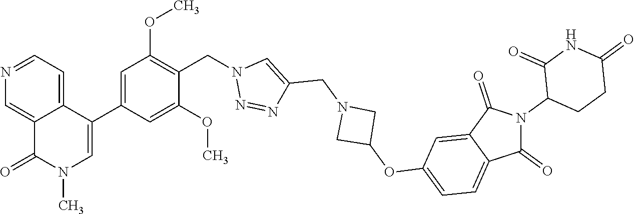 [SMILES: COc1cc(-c2cn(C)c(=O)c3cnccc23)cc(OC)c1Cn1cc(CN2CC(Oc3ccc4c(c3)C(=O)N(C3CCC(=O)NC3=O)C4=O)C2)nn1] |
| D271 | 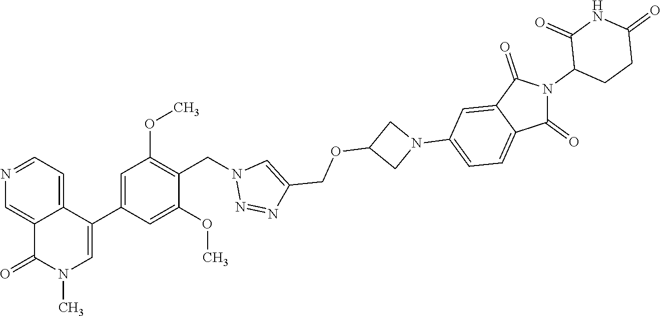 [SMILES: COc1cc(-c2cn(C)c(=O)c3cnccc23)cc(OC)c1Cn1cc(COC2CN(c3ccc4c(c3)C(=O)N(C3CCC(=O)NC3=O)C4=O)C2)nn1] |
| D272 | 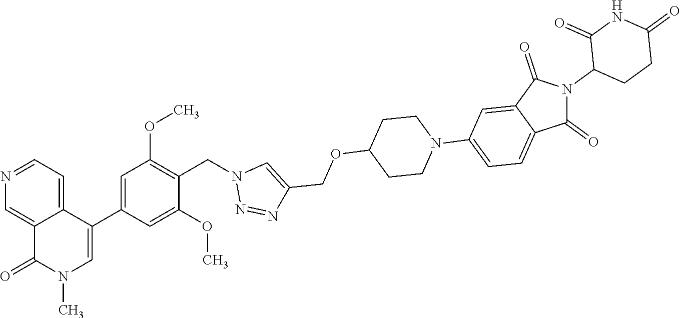 [SMILES: COc1cc(-c2cn(C)c(=O)c3cnccc23)cc(OC)c1Cn1cc(COC2CCN(c3ccc4c(c3)C(=O)N(C3CCC(=O)NC3=O)C4=O)CC2)nn1] |
| D273 | 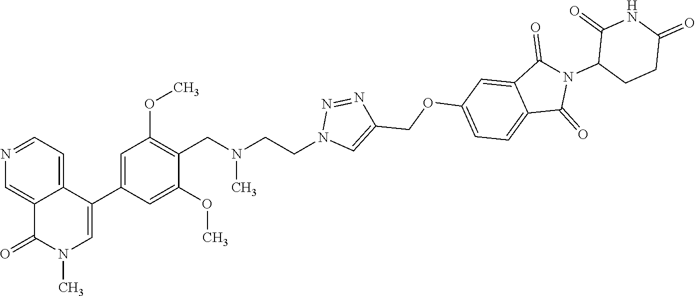 [SMILES: COc1cc(-c2cn(C)c(=O)c3cnccc23)cc(OC)c1CN(C)CCn1cc(COc2ccc3c(c2)C(=O)N(C2CCC(=O)NC2=O)C3=O)nn1] |
| D274 | 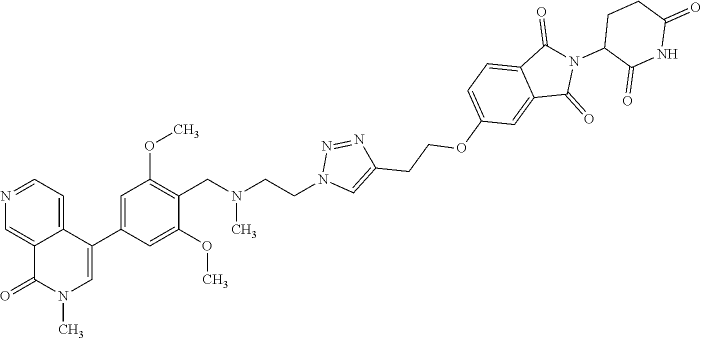 [SMILES: COc1cc(-c2cn(C)c(=O)c3cnccc23)cc(OC)c1CN(C)CCn1cc(CCOc2ccc3c(c2)C(=O)N(C2CCC(=O)NC2=O)C3=O)nn1] |
| D275 | 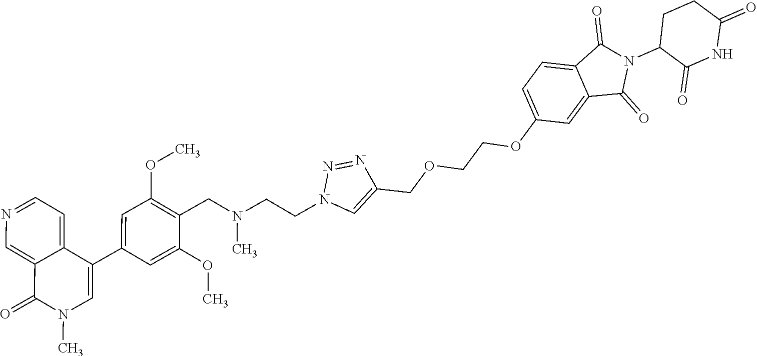 [SMILES: COc1cc(-c2cn(C)c(=O)c3cnccc23)cc(OC)c1CN(C)CCn1cc(COCCOc2ccc3c(c2)C(=O)N(C2CCC(=O)NC2=O)C3=O)nn1] |
| D276 | 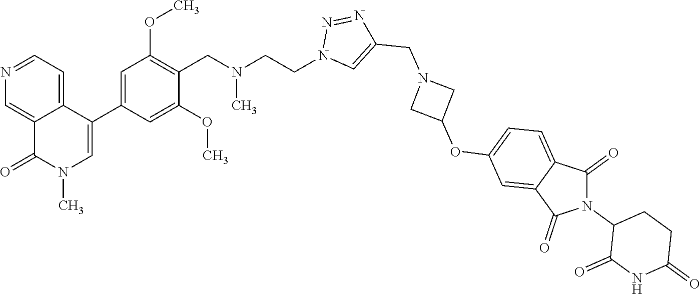 [SMILES: COc1cc(-c2cn(C)c(=O)c3cnccc23)cc(OC)c1CN(C)CCn1cc(CN2CC(Oc3ccc4c(c3)C(=O)N(C3CCC(=O)NC3=O)C4=O)C2)nn1] |
| D277 | 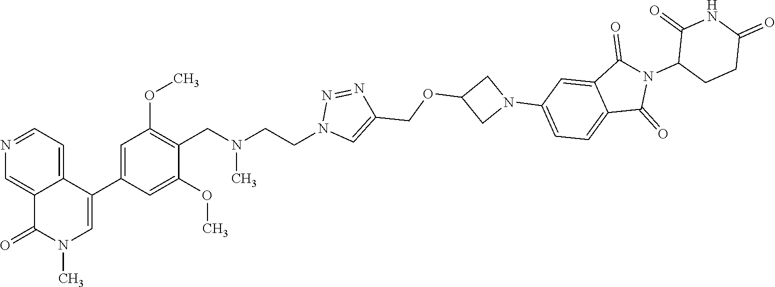 [SMILES: COc1cc(-c2cn(C)c(=O)c3cnccc23)cc(OC)c1CN(C)CCn1cc(COC2CN(c3ccc4c(c3)C(=O)N(C3CCC(=O)NC3=O)C4=O)C2)nn1] |
| D278 | 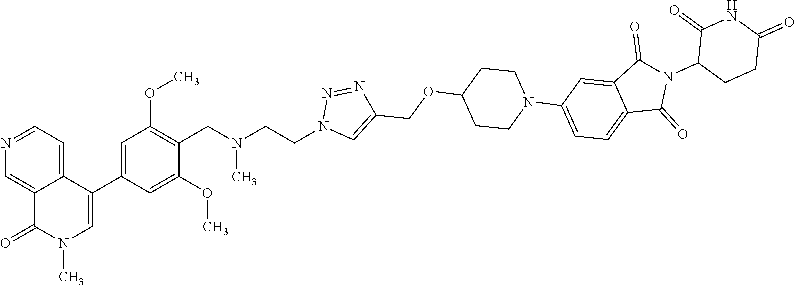 [SMILES: COc1cc(-c2cn(C)c(=O)c3cnccc23)cc(OC)c1CN(C)CCn1cc(COC2CCN(c3ccc4c(c3)C(=O)N(C3CCC(=O)NC3=O)C4=O)CC2)nn1] |
| D279 | 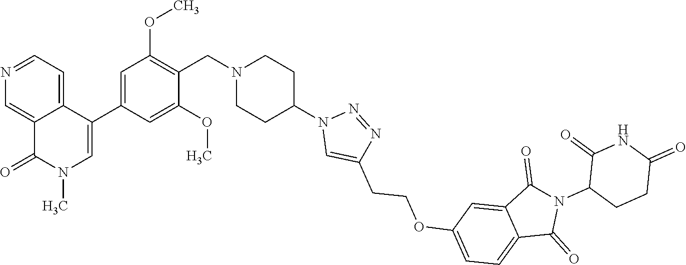 [SMILES: COc1cc(-c2cn(C)c(=O)c3cnccc23)cc(OC)c1CN1CCC(n2cc(CCOc3ccc4c(c3)C(=O)N(C3CCC(=O)NC3=O)C4=O)nn2)CC1] |
| D280 | 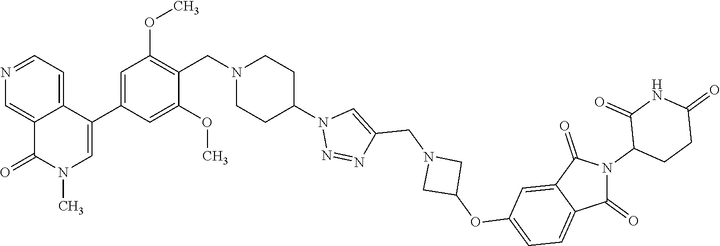 [SMILES: COc1cc(-c2cn(C)c(=O)c3cnccc23)cc(OC)c1CN1CCC(n2cc(CN3CC(Oc4ccc5c(c4)C(=O)N(C4CCC(=O)NC4=O)C5=O)C3)nn2)CC1] |
| D281 | 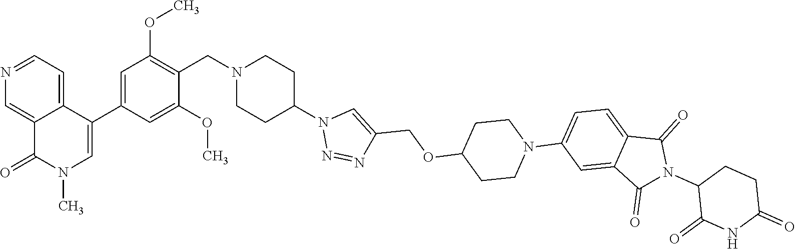 [SMILES: COc1cc(-c2cn(C)c(=O)c3cnccc23)cc(OC)c1CN1CCC(n2cc(COC3CCN(c4ccc5c(c4)C(=O)N(C4CCC(=O)NC4=O)C5=O)CC3)nn2)CC1] |
| D282 | 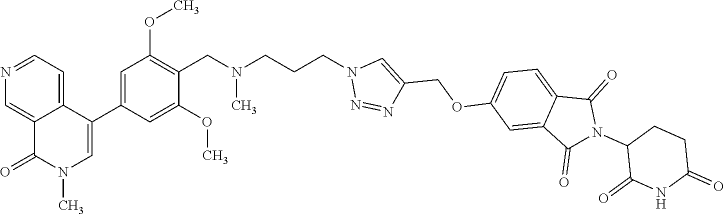 [SMILES: COc1cc(-c2cn(C)c(=O)c3cnccc23)cc(OC)c1CN(C)CCCn1cc(COc2ccc3c(c2)C(=O)N(C2CCC(=O)NC2=O)C3=O)nn1] |
| D283 | 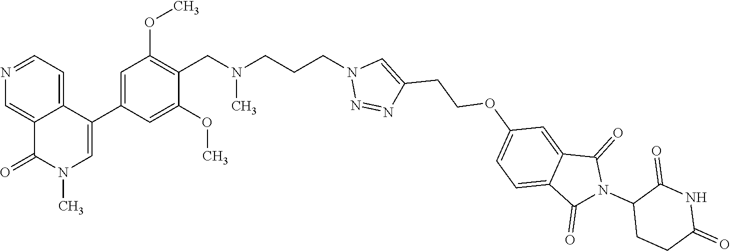 |
| D284 | 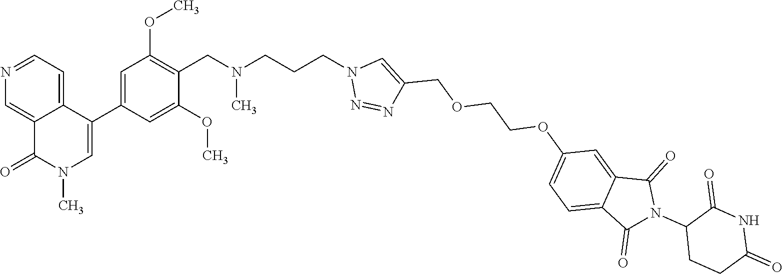 [SMILES: COc1cc(-c2cn(C)c(=O)c3cnccc23)cc(OC)c1CN(C)CCCn1cc(COCCOc2ccc3c(c2)C(=O)N(C2CCC(=O)NC2=O)C3=O)nn1] |
| D285 | 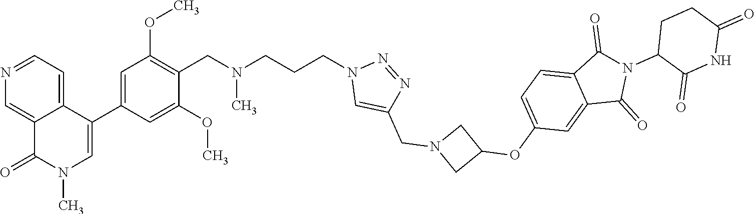 [SMILES: COc1cc(-c2cn(C)c(=O)c3cnccc23)cc(OC)c1CN(C)CCCn1cc(CN2CC(Oc3ccc4c(c3)C(=O)N(C3CCC(=O)NC3=O)C4=O)C2)nn1] |
| D286 | 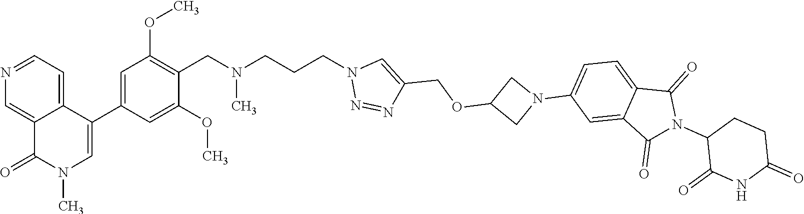 [SMILES: COc1cc(-c2cn(C)c(=O)c3cnccc23)cc(OC)c1CN(C)CCCn1cc(COC2CN(c3ccc4c(c3)C(=O)N(C3CCC(=O)NC3=O)C4=O)C2)nn1] |
| D287 | 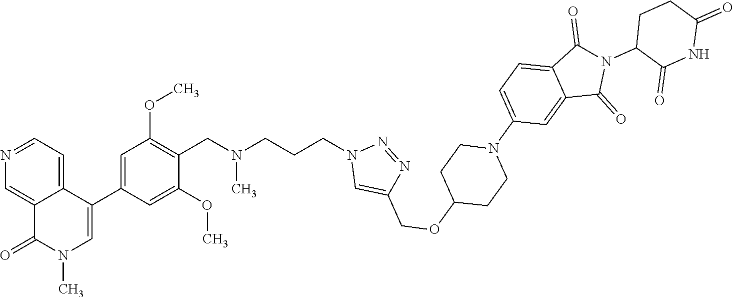 [SMILES: COc1cc(-c2cn(C)c(=O)c3cnccc23)cc(OC)c1CN(C)CCCn1cc(COC2CCN(c3ccc4c(c3)C(=O)N(C3CCC(=O)NC3=O)C4=O)CC2)nn1] |
| D288 | 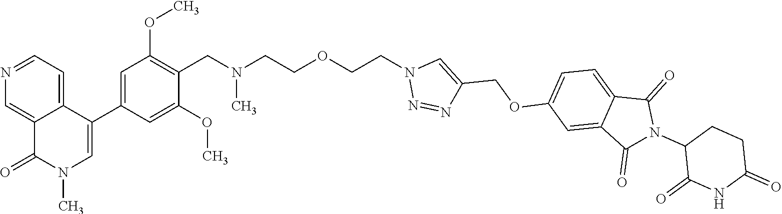 [SMILES: COc1cc(-c2cn(C)c(=O)c3cnccc23)cc(OC)c1CN(C)CCOCCn1cc(COc2ccc3c(c2)C(=O)N(C2CCC(=O)NC2=O)C3=O)nn1] |
| D289 | 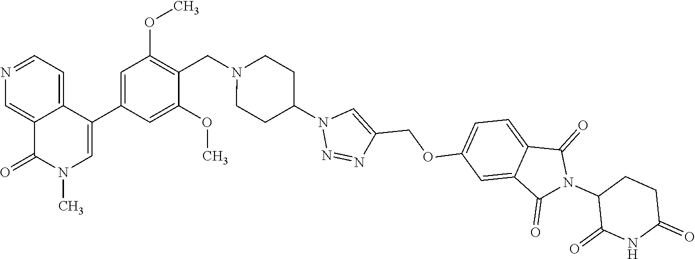 [SMILES: COc1cc(-c2cn(C)c(=O)c3cnccc23)cc(OC)c1CN1CCC(n2cc(COc3ccc4c(c3)C(=O)N(C3CCC(=O)NC3=O)C4=O)nn2)CC1] |
| D290 | 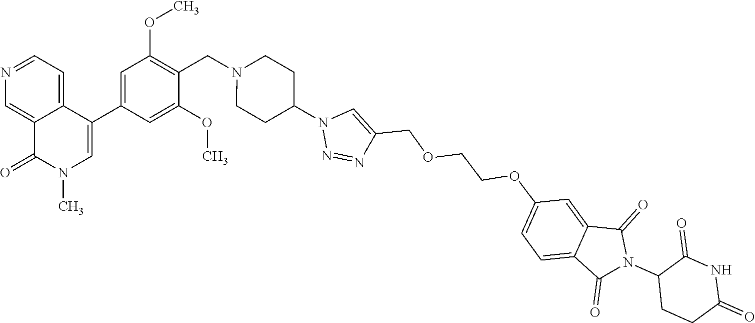 [SMILES: COc1cc(-c2cn(C)c(=O)c3cnccc23)cc(OC)c1CN1CCC(n2cc(COCCOc3ccc4c(c3)C(=O)N(C3CCC(=O)NC3=O)C4=O)nn2)CC1] |
| D291 | 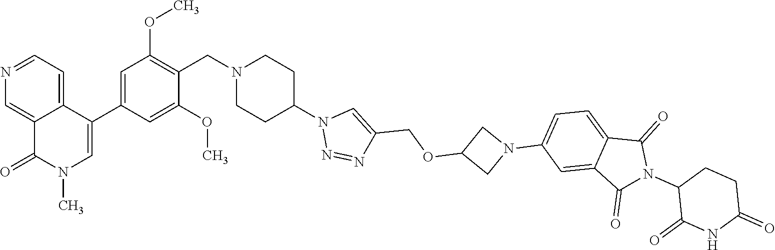 [SMILES: COc1cc(-c2cn(C)c(=O)c3cnccc23)cc(OC)c1CN1CCC(n2cc(COC3CN(c4ccc5c(c4)C(=O)N(C4CCC(=O)NC4=O)C5=O)C3)nn2)CC1] |
| D292 | 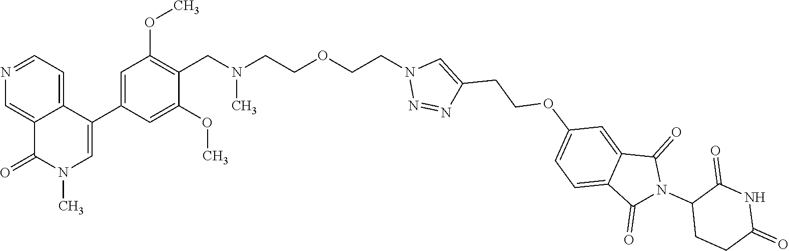 [SMILES: COc1cc(-c2cn(C)c(=O)c3cnccc23)cc(OC)c1CN(C)CCOCCn1cc(CCOc2ccc3c(c2)C(=O)N(C2CCC(=O)NC2=O)C3=O)nn1] |
| D293 | 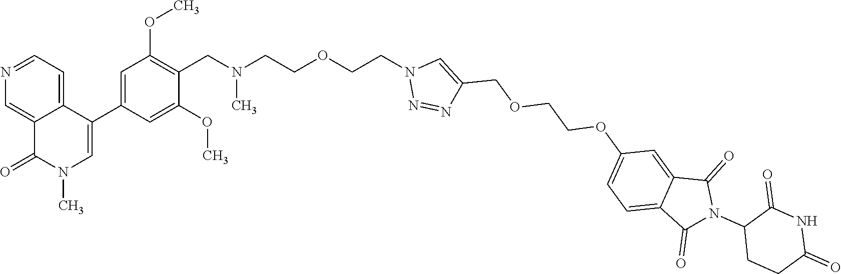 [SMILES: COc1cc(-c2cn(C)c(=O)c3cnccc23)cc(OC)c1CN(C)CCOCCn1cc(COCCOc2ccc3c(c2)C(=O)N(C2CCC(=O)NC2=O)C3=O)nn1] |
| D294 | 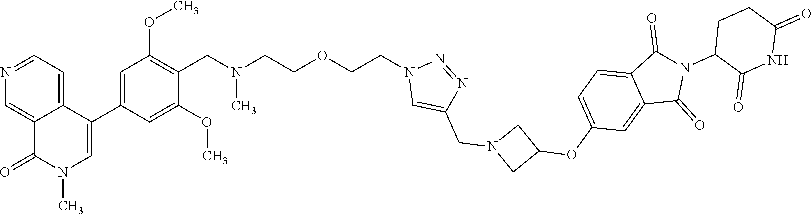 [SMILES: COc1cc(-c2cn(C)c(=O)c3cnccc23)cc(OC)c1CN(C)CCOCCn1cc(CN2CC(Oc3ccc4c(c3)C(=O)N(C3CCC(=O)NC3=O)C4=O)C2)nn1] |
| D295 | 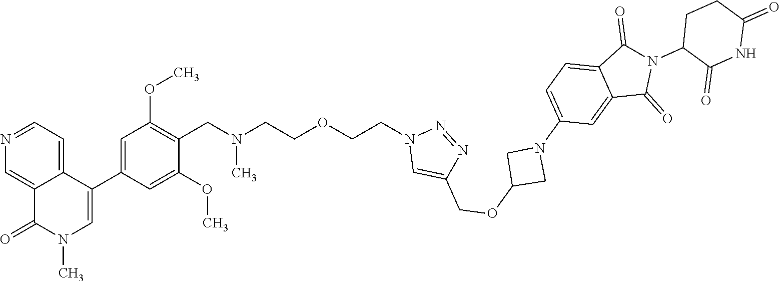 [SMILES: COc1cc(-c2cn(C)c(=O)c3cnccc23)cc(OC)c1CN(C)CCOCCn1cc(COC2CN(c3ccc4c(c3)C(=O)N(C3CCC(=O)NC3=O)C4=O)C2)nn1] |
| D296 | 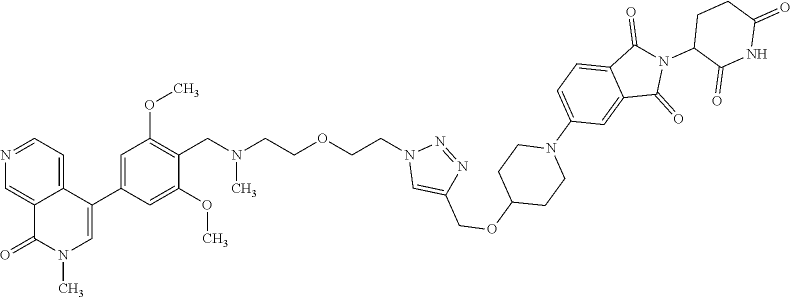 [SMILES: COc1cc(-c2cn(C)c(=O)c3cnccc23)cc(OC)c1CN(C)CCOCCn1cc(COC2CCN(c3ccc4c(c3)C(=O)N(C3CCC(=O)NC3=O)C4=O)CC2)nn1] |
| D297 | 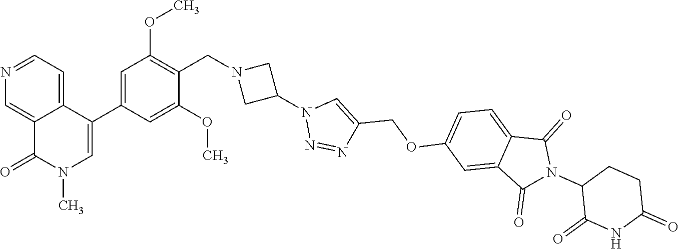 [SMILES: COc1cc(-c2cn(C)c(=O)c3cnccc23)cc(OC)c1CN1CC(n2cc(COc3ccc4c(c3)C(=O)N(C3CCC(=O)NC3=O)C4=O)nn2)C1] |
| D298 | 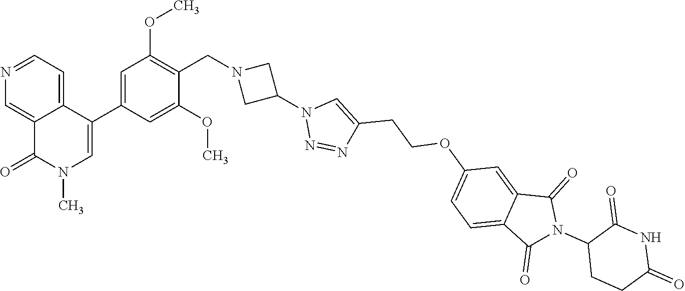 [SMILES: COc1cc(-c2cn(C)c(=O)c3cnccc23)cc(OC)c1CN1CC(n2cc(CCOc3ccc4c(c3)C(=O)N(C3CCC(=O)NC3=O)C4=O)nn2)C1] |
| D299 | 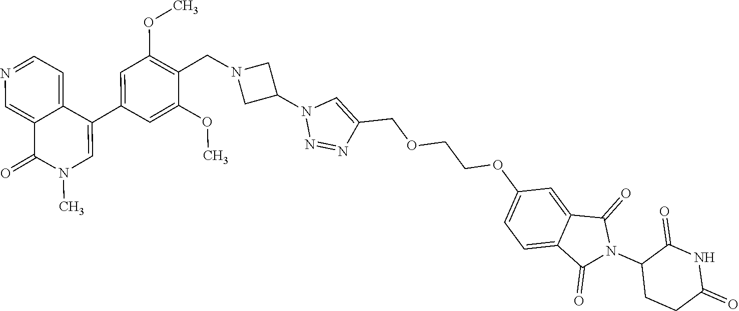 [SMILES: COc1cc(-c2cn(C)c(=O)c3cnccc23)cc(OC)c1CN1CC(n2cc(COCCOc3ccc4c(c3)C(=O)N(C3CCC(=O)NC3=O)C4=O)nn2)C1] |
| D300 | 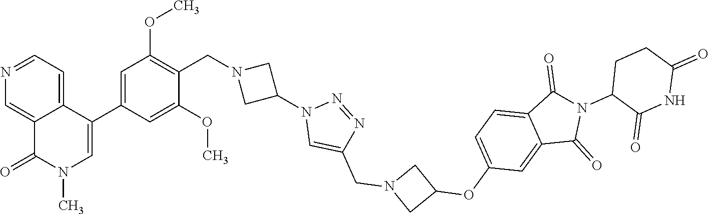 [SMILES: COc1cc(-c2cn(C)c(=O)c3cnccc23)cc(OC)c1CN1CC(n2cc(CN3CC(Oc4ccc5c(c4)C(=O)N(C4CCC(=O)NC4=O)C5=O)C3)nn2)C1] |
| D301 | 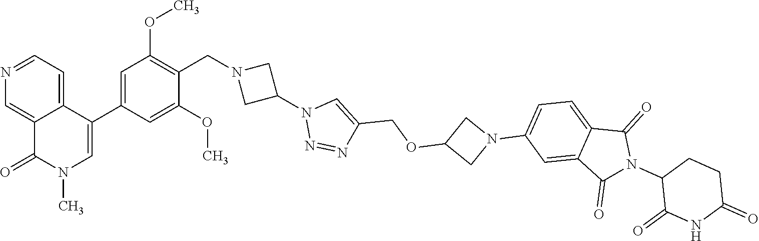 [SMILES: COc1cc(-c2cn(C)c(=O)c3cnccc23)cc(OC)c1CN1CC(n2cc(COC3CN(c4ccc5c(c4)C(=O)N(C4CCC(=O)NC4=O)C5=O)C3)nn2)C1] |
| D302 | 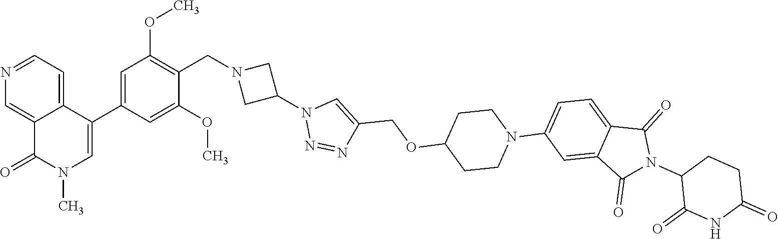 [SMILES: COc1cc(-c2cn(C)c(=O)c3cnccc23)cc(OC)c1CN1CC(n2cc(COC3CCN(c4ccc5c(c4)C(=O)N(C4CCC(=O)NC4=O)C5=O)CC3)nn2)C1] |
| D303 | 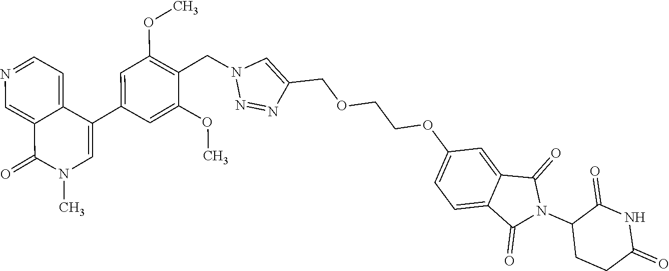 [SMILES: COc1cc(-c2cn(C)c(=O)c3cnccc23)cc(OC)c1Cn1cc(COCCOc2ccc3c(c2)C(=O)N(C2CCC(=O)NC2=O)C3=O)nn1] |
| D304 | 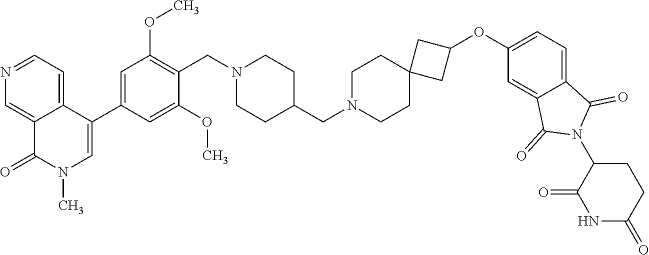 [SMILES: COc1cc(-c2cn(C)c(=O)c3cnccc23)cc(OC)c1CN1CCC(CN2CCC3(CC2)CC(Oc2ccc4c(c2)C(=O)N(C2CCC(=O)NC2=O)C4=O)C3)CC1] |
| D305 | 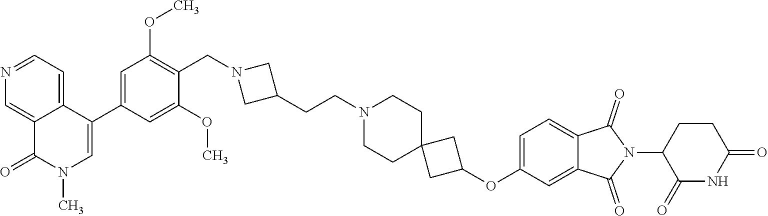 [SMILES: COc1cc(-c2cn(C)c(=O)c3cnccc23)cc(OC)c1CN1CC(CCN2CCC3(CC2)CC(Oc2ccc4c(c2)C(=O)N(C2CCC(=O)NC2=O)C4=O)C3)C1] |
| D306 | 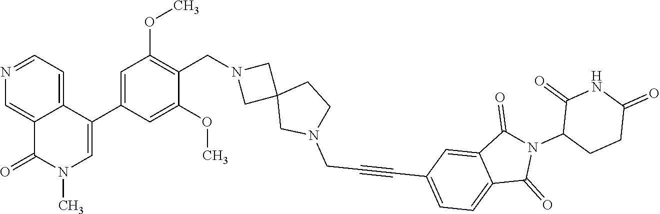 [SMILES: COc1cc(-c2cn(C)c(=O)c3cnccc23)cc(OC)c1CN1CC2(CCN(CC#Cc3ccc4c(c3)C(=O)N(C3CCC(=O)NC3=O)C4=O)C2)C1] |
| D307 | 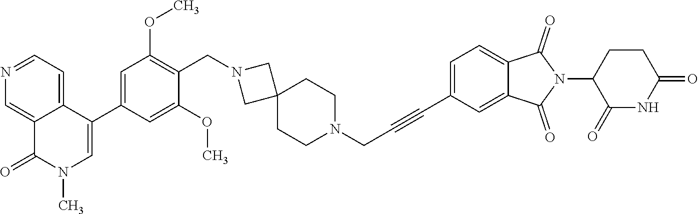 [SMILES: COc1cc(-c2cn(C)c(=O)c3cnccc23)cc(OC)c1CN1CC2(CCN(CC#Cc3ccc4c(c3)C(=O)N(C3CCC(=O)NC3=O)C4=O)CC2)C1] |
| D308 | 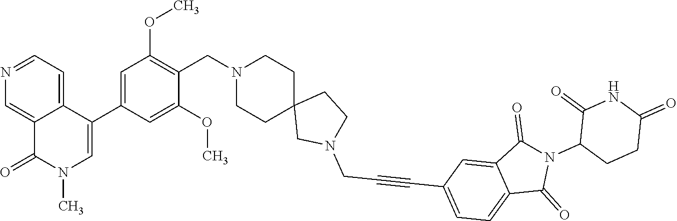 [SMILES: COc1cc(-c2cn(C)c(=O)c3cnccc23)cc(OC)c1CN1CCC2(CC1)CCN(CC#Cc1ccc3c(c1)C(=O)N(C1CCC(=O)NC1=O)C3=O)C2] |
| D309 | 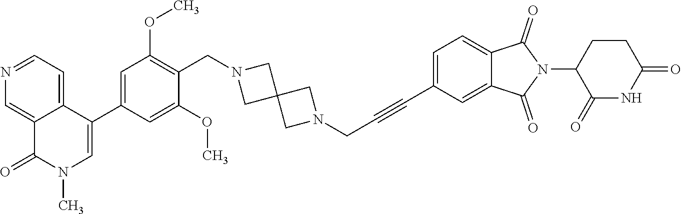 [SMILES: COc1cc(-c2cn(C)c(=O)c3cnccc23)cc(OC)c1CN1CC2(CN(CC#Cc3ccc4c(c3)C(=O)N(C3CCC(=O)NC3=O)C4=O)C2)C1] |
| D310 | 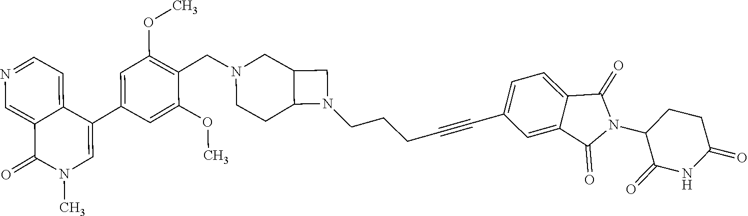 [SMILES: COc1cc(-c2cn(C)c(=O)c3cnccc23)cc(OC)c1CN1CCC2C(C1)CN2CCCC#Cc1ccc2c(c1)C(=O)N(C1CCC(=O)NC1=O)C2=O] |
| D311 | 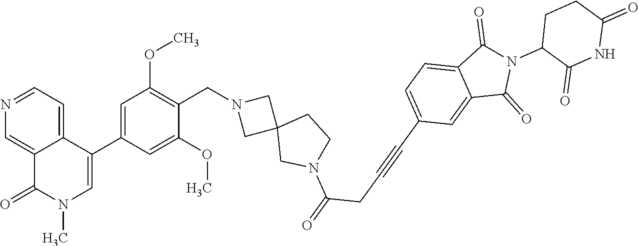 [SMILES: COc1cc(-c2cn(C)c(=O)c3cnccc23)cc(OC)c1CN1CC2(CCN(C(=O)CC#Cc3ccc4c(c3)C(=O)N(C3CCC(=O)NC3=O)C4=O)C2)C1] |
| D312 | 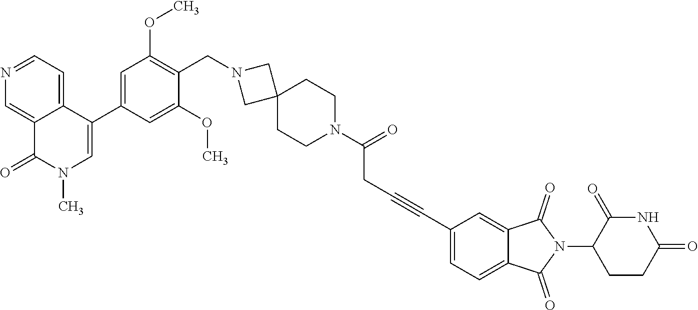 [SMILES: COc1cc(-c2cn(C)c(=O)c3cnccc23)cc(OC)c1CN1CC2(CCN(C(=O)CC#Cc3ccc4c(c3)C(=O)N(C3CCC(=O)NC3=O)C4=O)CC2)C1] |
| D313 | 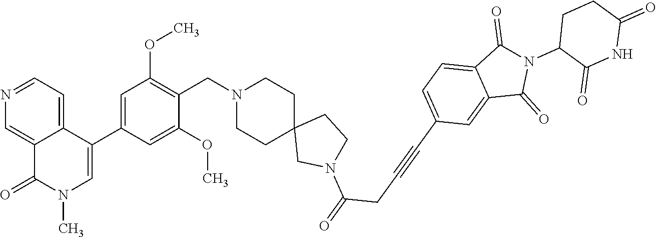 [SMILES: COc1cc(-c2cn(C)c(=O)c3cnccc23)cc(OC)c1CN1CCC2(CC1)CCN(C(=O)CC#Cc1ccc3c(c1)C(=O)N(C1CCC(=O)NC1=O)C3=O)C2] |
| D314 | 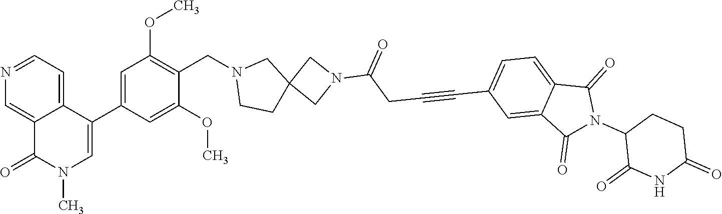 [SMILES: COc1cc(-c2cn(C)c(=O)c3cnccc23)cc(OC)c1CN1CCC2(C1)CN(C(=O)CC#Cc1ccc3c(c1)C(=O)N(C1CCC(=O)NC1=O)C3=O)C2] |
| D315 | 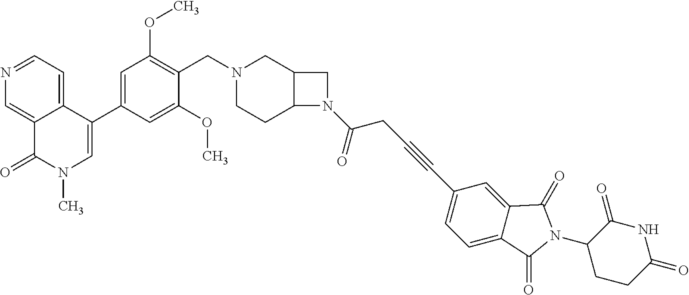 [SMILES: COc1cc(-c2cn(C)c(=O)c3cnccc23)cc(OC)c1CN1CCC2C(C1)CN2C(=O)CC#Cc1ccc2c(c1)C(=O)N(C1CCC(=O)NC1=O)C2=O] |
| D316 | 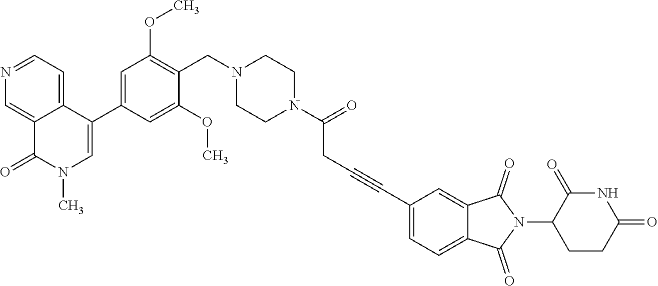 [SMILES: COc1cc(-c2cn(C)c(=O)c3cnccc23)cc(OC)c1CN1CCN(C(=O)CC#Cc2ccc3c(c2)C(=O)N(C2CCC(=O)NC2=O)C3=O)CC1] |
| D317 | 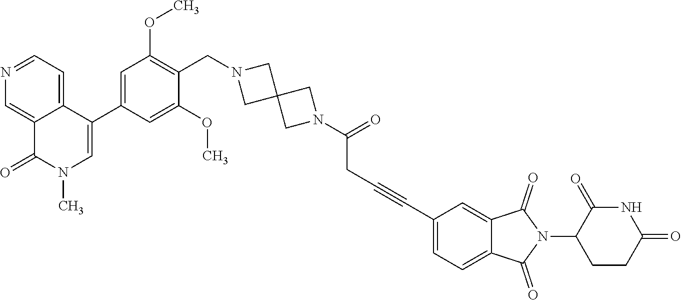 [SMILES: COc1cc(-c2cn(C)c(=O)c3cnccc23)cc(OC)c1CN1CC2(C1)CN(C(=O)CC#Cc1ccc3c(c1)C(=O)N(C1CCC(=O)NC1=O)C3=O)C2] |
| D318 | 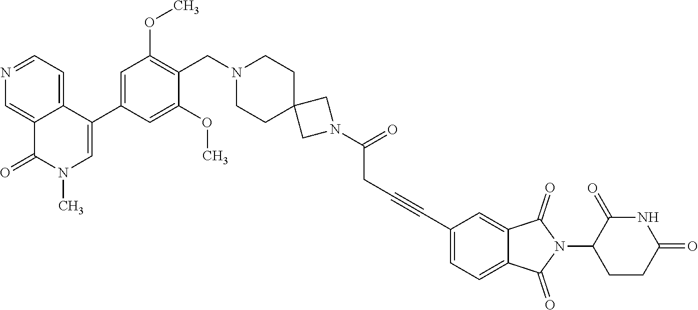 [SMILES: COc1cc(-c2cn(C)c(=O)c3cnccc23)cc(OC)c1CN1CCC2(CC1)CN(C(=O)CC#Cc1ccc3c(c1)C(=O)N(C1CCC(=O)NC1=O)C3=O)C2] |
| D319 | 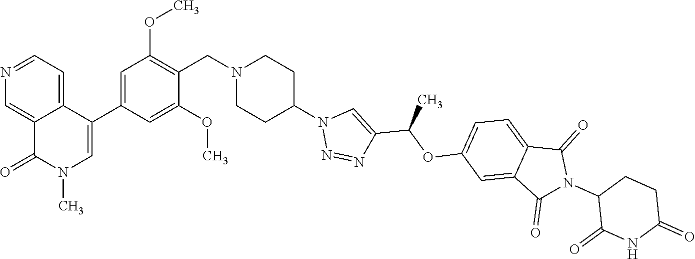 [SMILES: COc1cc(-c2cn(C)c(=O)c3cnccc23)cc(OC)c1CN1CCC(n2cc([C@@H](C)Oc3ccc4c(c3)C(=O)N(C3CCC(=O)NC3=O)C4=O)nn2)CC1] |
| D320 | 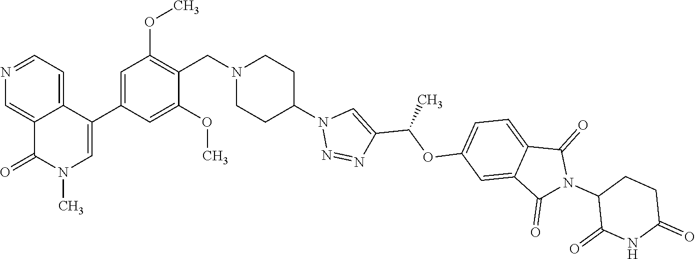 [SMILES: COc1cc(-c2cn(C)c(=O)c3cnccc23)cc(OC)c1CN1CCC(n2cc([C@H](C)Oc3ccc4c(c3)C(=O)N(C3CCC(=O)NC3=O)C4=O)nn2)CC1] |
| D321 | 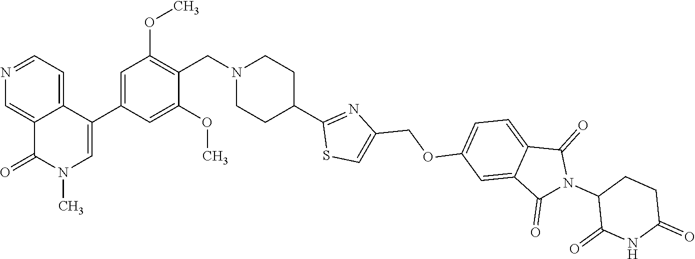 [SMILES: COc1cc(-c2cn(C)c(=O)c3cnccc23)cc(OC)c1CN1CCC(c2nc(COc3ccc4c(c3)C(=O)N(C3CCC(=O)NC3=O)C4=O)cs2)CC1] |
| D322 | 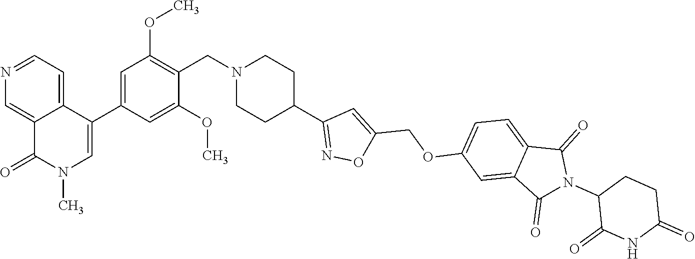 [SMILES: COc1cc(-c2cn(C)c(=O)c3cnccc23)cc(OC)c1CN1CCC(c2cc(COc3ccc4c(c3)C(=O)N(C3CCC(=O)NC3=O)C4=O)on2)CC1] |
| D323 | 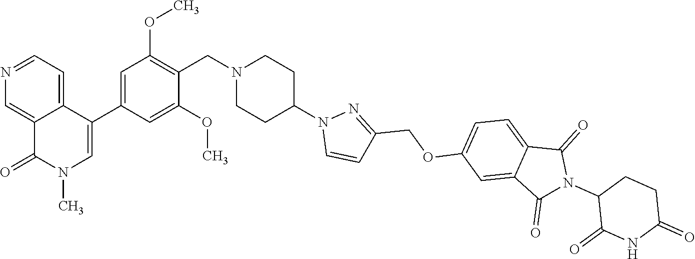 [SMILES: COc1cc(-c2cn(C)c(=O)c3cnccc23)cc(OC)c1CN1CCC(n2ccc(COc3ccc4c(c3)C(=O)N(C3CCC(=O)NC3=O)C4=O)n2)CC1] |
| D324 | 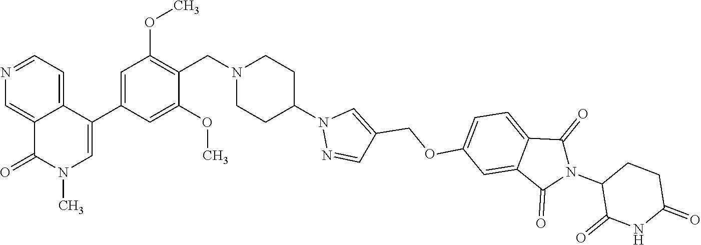 [SMILES: COc1cc(-c2cn(C)c(=O)c3cnccc23)cc(OC)c1CN1CCC(n2cc(COc3ccc4c(c3)C(=O)N(C3CCC(=O)NC3=O)C4=O)cn2)CC1] |
| D325 | 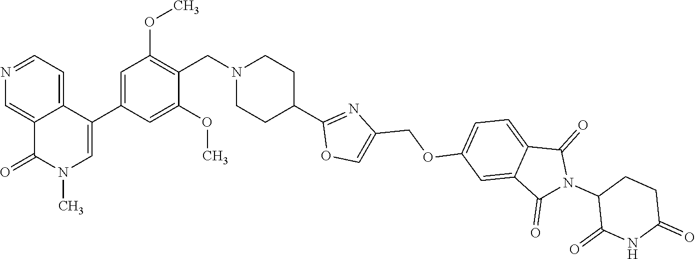 [SMILES: COc1cc(-c2cn(C)c(=O)c3cnccc23)cc(OC)c1CN1CCC(c2nc(COc3ccc4c(c3)C(=O)N(C3CCC(=O)NC3=O)C4=O)co2)CC1] |
| D326 | 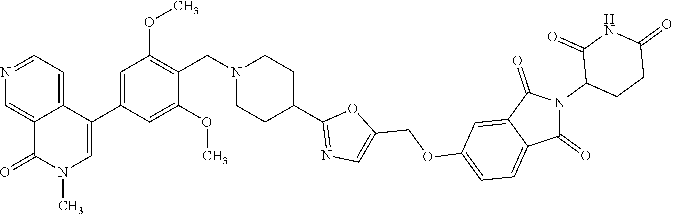 [SMILES: COc1cc(-c2cn(C)c(=O)c3cnccc23)cc(OC)c1CN1CCC(c2ncc(COc3ccc4c(c3)C(=O)N(C3CCC(=O)NC3=O)C4=O)o2)CC1] |
| D327 | 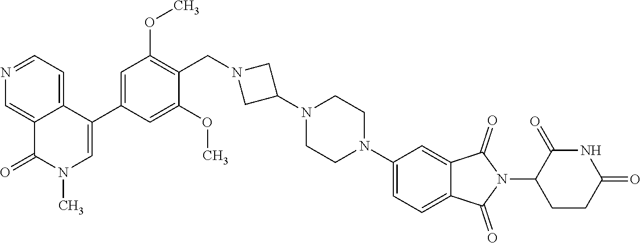 [SMILES: COc1cc(-c2cn(C)c(=O)c3cnccc23)cc(OC)c1CN1CC(N2CCN(c3ccc4c(c3)C(=O)N(C3CCC(=O)NC3=O)C4=O)CC2)C1] |
| D328 | 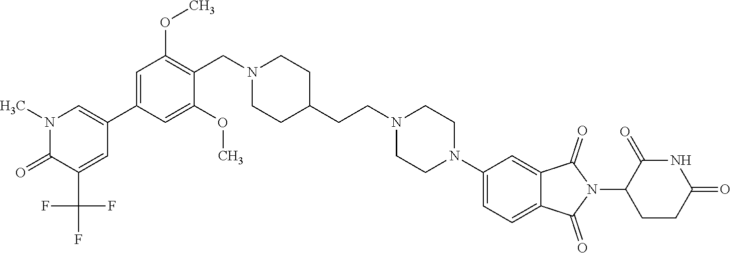 [SMILES: COc1cc(-c2cc(C(F)(F)F)c(=O)n(C)c2)cc(OC)c1CN1CCC(CCN2CCN(c3ccc4c(c3)C(=O)N(C3CCC(=O)NC3=O)C4=O)CC2)CC1] |
| D329 | 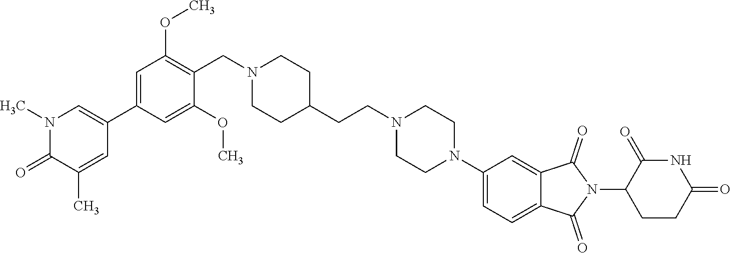 [SMILES: COc1cc(-c2cc(C)c(=O)n(C)c2)cc(OC)c1CN1CCC(CCN2CCN(c3ccc4c(c3)C(=O)N(C3CCC(=O)NC3=O)C4=O)CC2)CC1] |
| D330 | 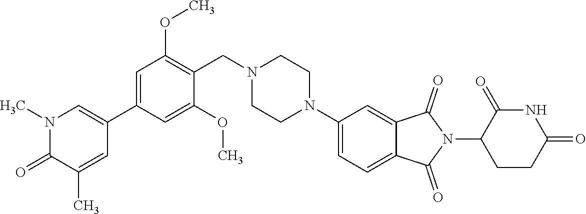 [SMILES: COc1cc(-c2cc(C)c(=O)n(C)c2)cc(OC)c1CN1CCN(c2ccc3c(c2)C(=O)N(C2CCC(=O)NC2=O)C3=O)CC1] |
| D331 | 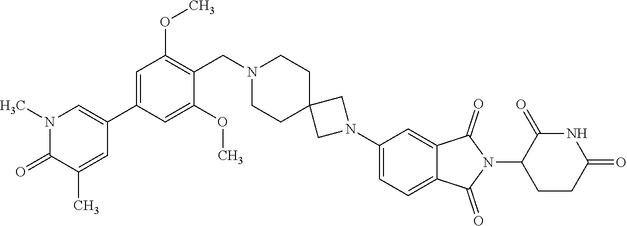 [SMILES: COc1cc(-c2cc(C)c(=O)n(C)c2)cc(OC)c1CN1CCC2(CC1)CN(c1ccc3c(c1)C(=O)N(C1CCC(=O)NC1=O)C3=O)C2] |
| D332 | 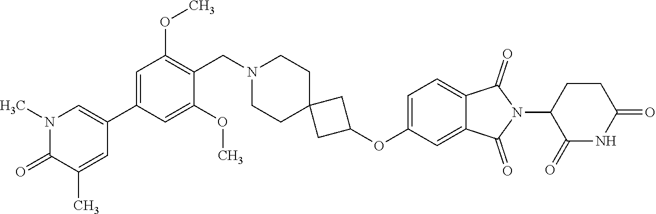 [SMILES: COc1cc(-c2cc(C)c(=O)n(C)c2)cc(OC)c1CN1CCC2(CC1)CC(Oc1ccc3c(c1)C(=O)N(C1CCC(=O)NC1=O)C3=O)C2] |
| D333 | 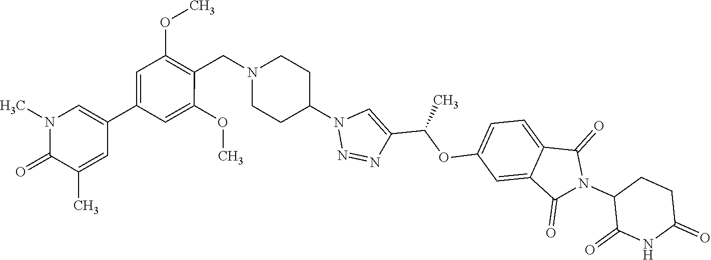 [SMILES: COc1cc(-c2cc(C)c(=O)n(C)c2)cc(OC)c1CN1CCC(n2cc([C@H](C)Oc3ccc4c(c3)C(=O)N(C3CCC(=O)NC3=O)C4=O)nn2)CC1] |
| D334 | 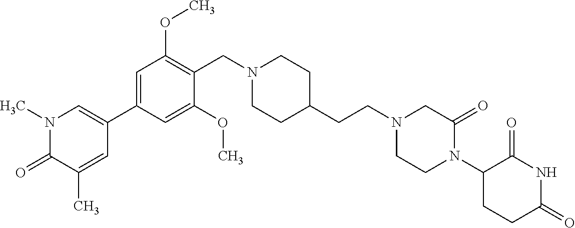 [SMILES: COc1cc(-c2cc(C)c(=O)n(C)c2)cc(OC)c1CN1CCC(CCN2CCN(C3CCC(=O)NC3=O)C(=O)C2)CC1] |
| D335 | 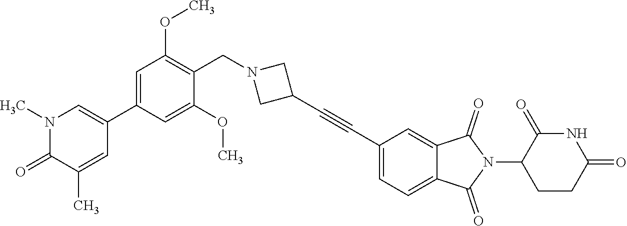 [SMILES: COc1cc(-c2cc(C)c(=O)n(C)c2)cc(OC)c1CN1CC(C#Cc2ccc3c(c2)C(=O)N(C2CCC(=O)NC2=O)C3=O)C1] |
| D336 | 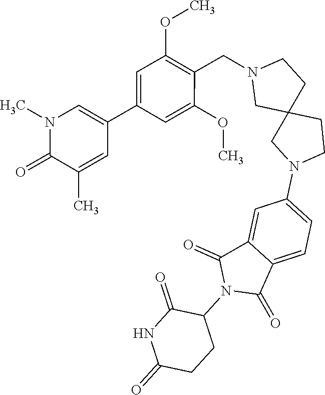 [SMILES: COc1cc(-c2cc(C)c(=O)n(C)c2)cc(OC)c1CN1CCC2(CCN(c3ccc4c(c3)C(=O)N(C3CCC(=O)NC3=O)C4=O)C2)C1] |
| D337 | 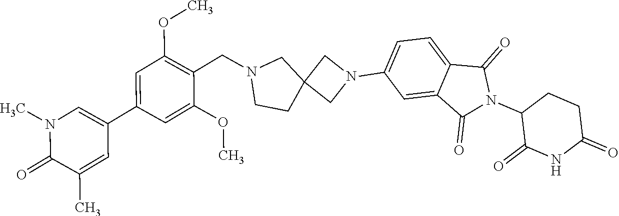 [SMILES: COc1cc(-c2cc(C)c(=O)n(C)c2)cc(OC)c1CN1CCC2(C1)CN(c1ccc3c(c1)C(=O)N(C1CCC(=O)NC1=O)C3=O)C2] |
| D338 | 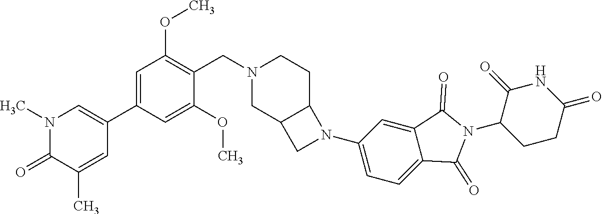 [SMILES: COc1cc(-c2cc(C)c(=O)n(C)c2)cc(OC)c1CN1CCC2C(C1)CN2c1ccc2c(c1)C(=O)N(C1CCC(=O)NC1=O)C2=O] |
| D339 | 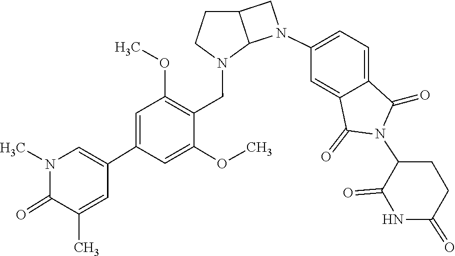 [SMILES: COc1cc(-c2cc(C)c(=O)n(C)c2)cc(OC)c1CN1CCC2CN(c3ccc4c(c3)C(=O)N(C3CCC(=O)NC3=O)C4=O)C21] |
| D340 | 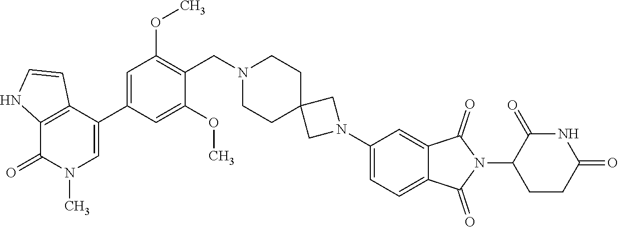 [SMILES: COc1cc(-c2cn(C)c(=O)c3[nH]ccc23)cc(OC)c1CN1CCC2(CC1)CN(c1ccc3c(c1)C(=O)N(C1CCC(=O)NC1=O)C3=O)C2] |
| D341 | 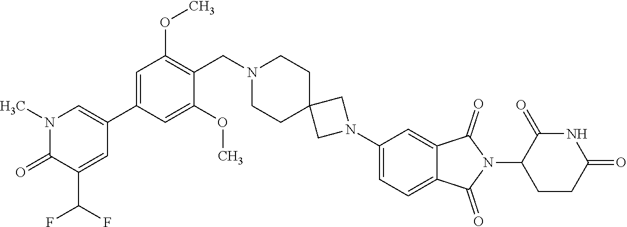 [SMILES: COc1cc(-c2cc(C(F)F)c(=O)n(C)c2)cc(OC)c1CN1CCC2(CC1)CN(c1ccc3c(c1)C(=O)N(C1CCC(=O)NC1=O)C3=O)C2] |
| D342 | 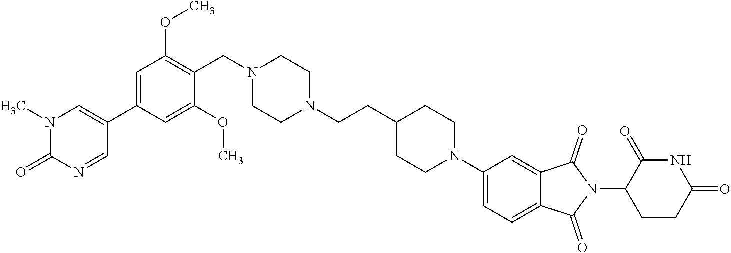 [SMILES: COc1cc(-c2cnc(=O)n(C)c2)cc(OC)c1CN1CCN(CCC2CCN(c3ccc4c(c3)C(=O)N(C3CCC(=O)NC3=O)C4=O)CC2)CC1] |
| D343 | 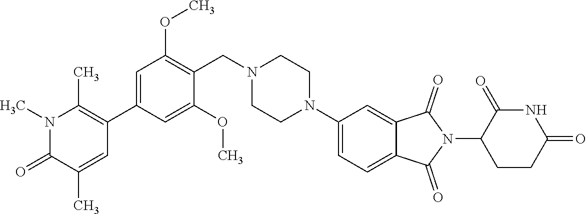 [SMILES: COc1cc(-c2cc(C)c(=O)n(C)c2C)cc(OC)c1CN1CCN(c2ccc3c(c2)C(=O)N(C2CCC(=O)NC2=O)C3=O)CC1] |
| D344 | 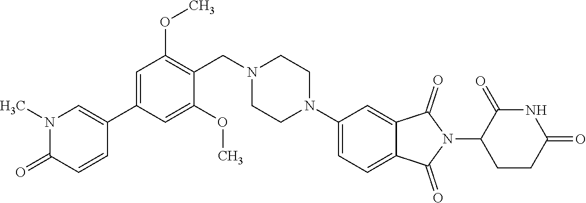 [SMILES: COc1cc(-c2ccc(=O)n(C)c2)cc(OC)c1CN1CCN(c2ccc3c(c2)C(=O)N(C2CCC(=O)NC2=O)C3=O)CC1] |
| D345 | 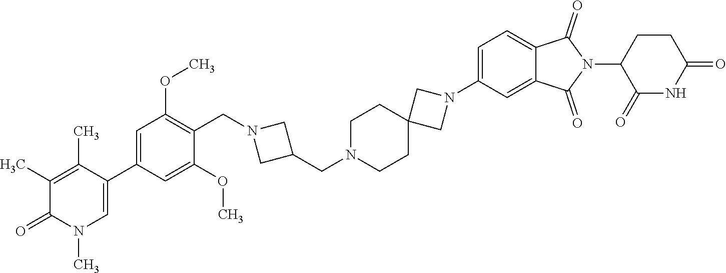 [SMILES: COc1cc(-c2cn(C)c(=O)c(C)c2C)cc(OC)c1CN1CC(CN2CCC3(CC2)CN(c2ccc4c(c2)C(=O)N(C2CCC(=O)NC2=O)C4=O)C3)C1] |
| D346 | 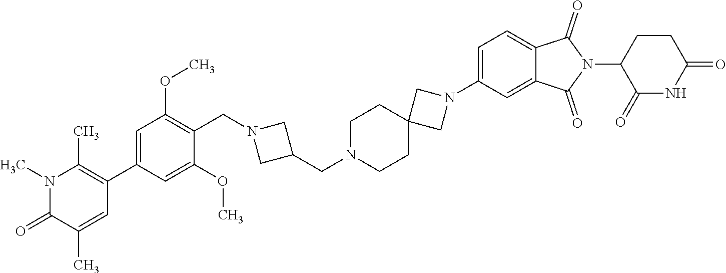 [SMILES: COc1cc(-c2cc(C)c(=O)n(C)c2C)cc(OC)c1CN1CC(CN2CCC3(CC2)CN(c2ccc4c(c2)C(=O)N(C2CCC(=O)NC2=O)C4=O)C3)C1] |
| D347 | 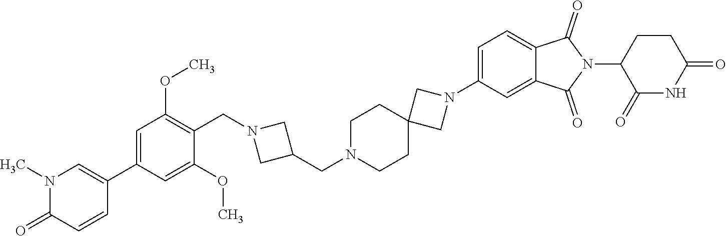 [SMILES: COc1cc(-c2ccc(=O)n(C)c2)cc(OC)c1CN1CC(CN2CCC3(CC2)CN(c2ccc4c(c2)C(=O)N(C2CCC(=O)NC2=O)C4=O)C3)C1] |
| D348 | 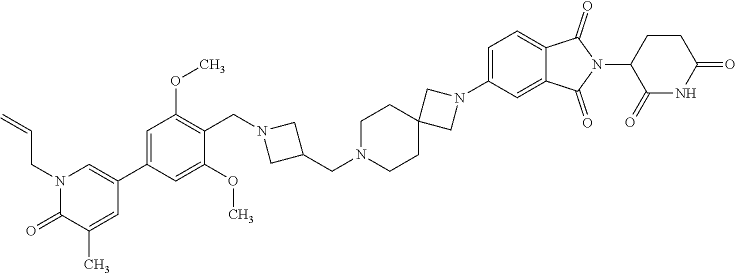 [SMILES: C=CCn1cc(-c2cc(OC)c(CN3CC(CN4CCC5(CC4)CN(c4ccc6c(c4)C(=O)N(C4CCC(=O)NC4=O)C6=O)C5)C3)c(OC)c2)cc(C)c1=O] |
| D349 | 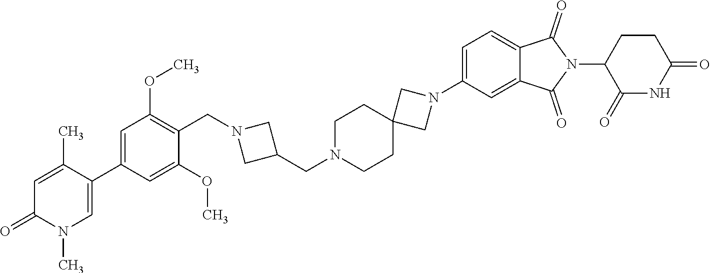 [SMILES: COc1cc(-c2cn(C)c(=O)cc2C)cc(OC)c1CN1CC(CN2CCC3(CC2)CN(c2ccc4c(c2)C(=O)N(C2CCC(=O)NC2=O)C4=O)C3)C1] |
| D350 | 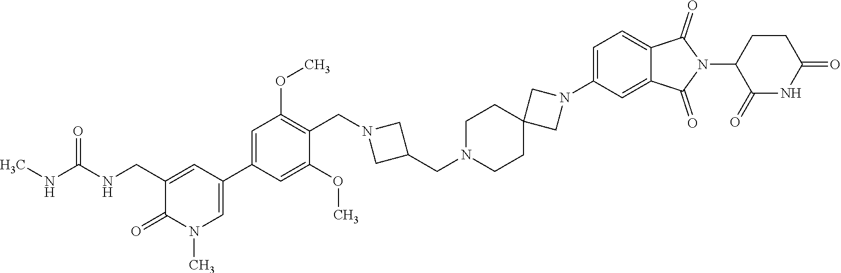 [SMILES: CNC(=O)NCc1cc(-c2cc(OC)c(CN3CC(CN4CCC5(CC4)CN(c4ccc6c(c4)C(=O)N(C4CCC(=O)NC4=O)C6=O)C5)C3)c(OC)c2)cn(C)c1=O] |
| D351 | 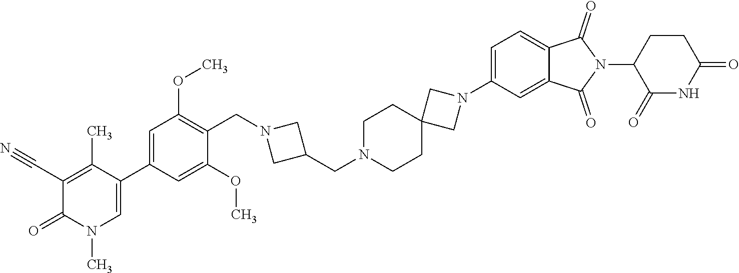 [SMILES: COc1cc(-c2cn(C)c(=O)c(C#N)c2C)cc(OC)c1CN1CC(CN2CCC3(CC2)CN(c2ccc4c(c2)C(=O)N(C2CCC(=O)NC2=O)C4=O)C3)C1] |
| D352 | 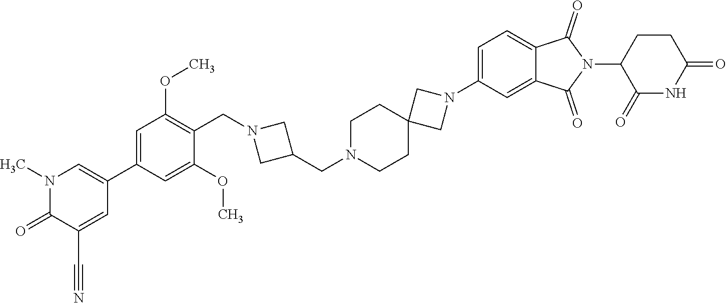 [SMILES: COc1cc(-c2cc(C#N)c(=O)n(C)c2)cc(OC)c1CN1CC(CN2CCC3(CC2)CN(c2ccc4c(c2)C(=O)N(C2CCC(=O)NC2=O)C4=O)C3)C1] |
| D353 | 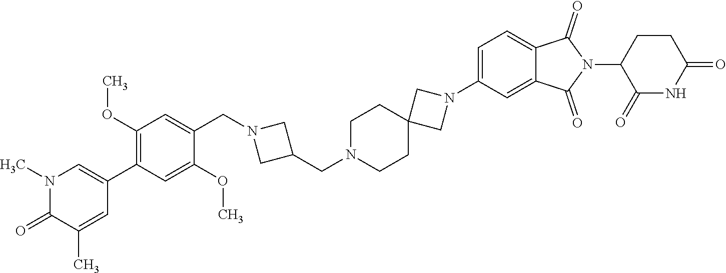 [SMILES: COc1cc(-c2cc(C)c(=O)n(C)c2)c(OC)cc1CN1CC(CN2CCC3(CC2)CN(c2ccc4c(c2)C(=O)N(C2CCC(=O)NC2=O)C4=O)C3)C1] |
| D354 | 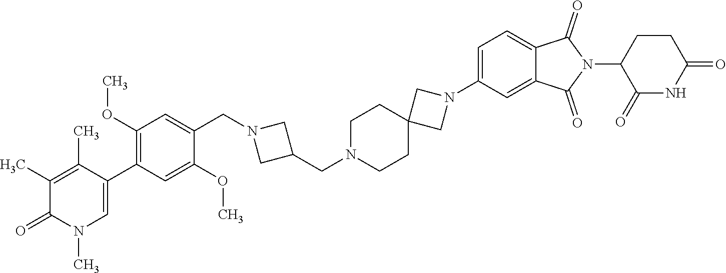 [SMILES: COc1cc(-c2cn(C)c(=O)c(C)c2C)c(OC)cc1CN1CC(CN2CCC3(CC2)CN(c2ccc4c(c2)C(=O)N(C2CCC(=O)NC2=O)C4=O)C3)C1] |
| D355 | 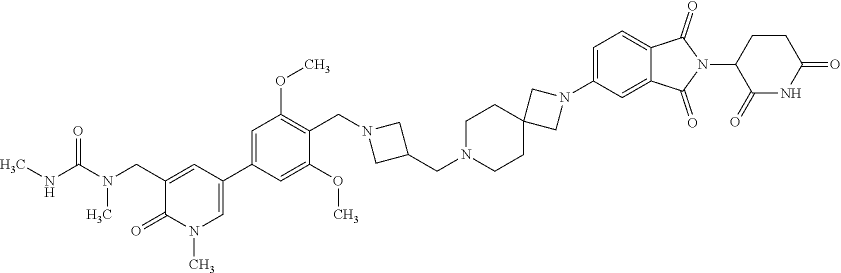 [SMILES: CNC(=O)N(C)Cc1cc(-c2cc(OC)c(CN3CC(CN4CCC5(CC4)CN(c4ccc6c(c4)C(=O)N(C4CCC(=O)NC4=O)C6=O)C5)C3)c(OC)c2)cn(C)c1=O] |
| D356 | 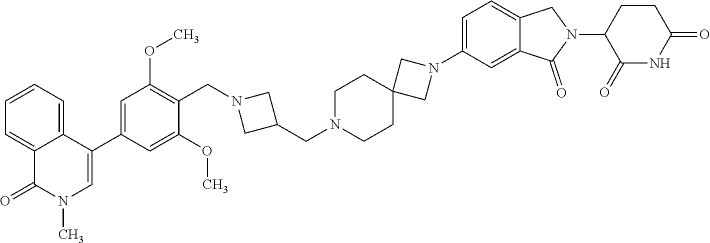 [SMILES: COc1cc(-c2cn(C)c(=O)c3ccccc23)cc(OC)c1CN1CC(CN2CCC3(CC2)CN(c2ccc4c(c2)C(=O)N(C2CCC(=O)NC2=O)C4)C3)C1] |
| D357 | 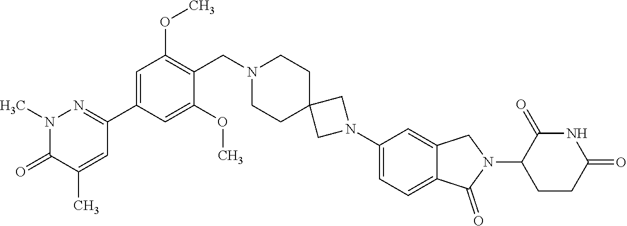 [SMILES: COc1cc(-c2cc(C)c(=O)n(C)n2)cc(OC)c1CN1CCC2(CC1)CN(c1ccc3c(c1)CN(C1CCC(=O)NC1=O)C3=O)C2] |
| D358 | 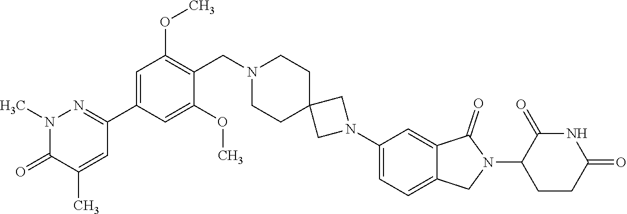 [SMILES: COc1cc(-c2cc(C)c(=O)n(C)n2)cc(OC)c1CN1CCC2(CC1)CN(c1ccc3c(c1)C(=O)N(C1CCC(=O)NC1=O)C3)C2] |
| D359 | 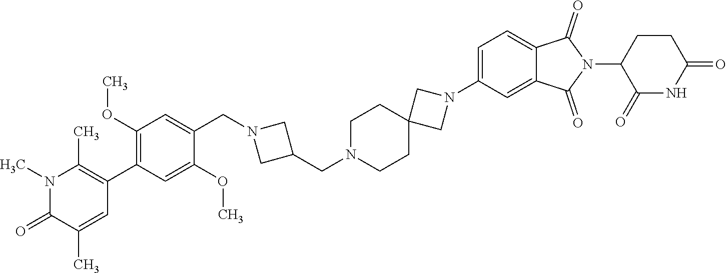 [SMILES: COc1cc(-c2cc(C)c(=O)n(C)c2C)c(OC)cc1CN1CC(CN2CCC3(CC2)CN(c2ccc4c(c2)C(=O)N(C2CCC(=O)NC2=O)C4=O)C3)C1] |
| D360 | 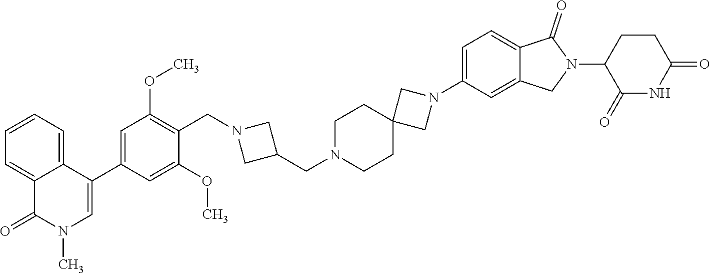 [SMILES: COc1cc(-c2cn(C)c(=O)c3ccccc23)cc(OC)c1CN1CC(CN2CCC3(CC2)CN(c2ccc4c(c2)CN(C2CCC(=O)NC2=O)C4=O)C3)C1] |
| D361 | 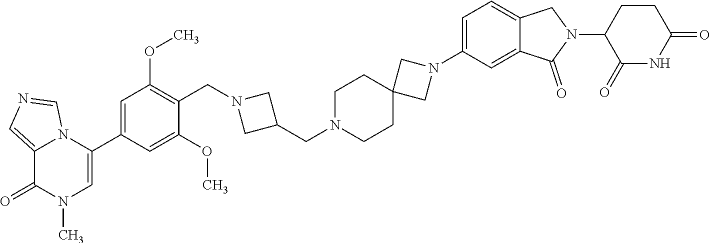 [SMILES: COc1cc(-c2cn(C)c(=O)c3cncn23)cc(OC)c1CN1CC(CN2CCC3(CC2)CN(c2ccc4c(c2)C(=O)N(C2CCC(=O)NC2=O)C4)C3)C1] |
| D362 | 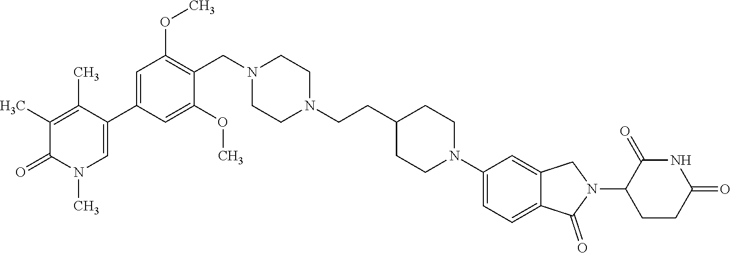 [SMILES: COc1cc(-c2cn(C)c(=O)c(C)c2C)cc(OC)c1CN1CCN(CCC2CCN(c3ccc4c(c3)CN(C3CCC(=O)NC3=O)C4=O)CC2)CC1] |
| D363 | 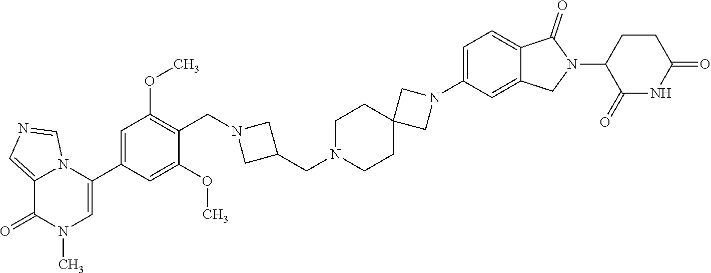 [SMILES: COc1cc(-c2cn(C)c(=O)c3cncn23)cc(OC)c1CN1CC(CN2CCC3(CC2)CN(c2ccc4c(c2)CN(C2CCC(=O)NC2=O)C4=O)C3)C1] |
| D364 | 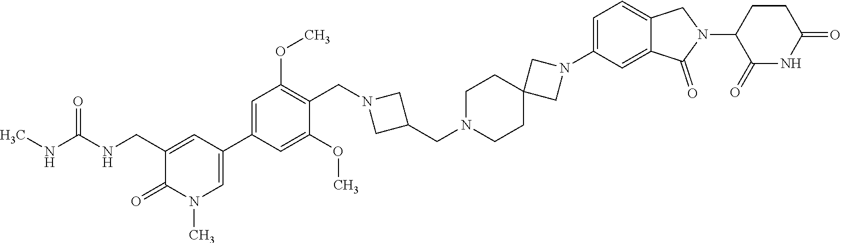 [SMILES: CNC(=O)NCc1cc(-c2cc(OC)c(CN3CC(CN4CCC5(CC4)CN(c4ccc6c(c4)C(=O)N(C4CCC(=O)NC4=O)C6)C5)C3)c(OC)c2)cn(C)c1=O] |
| D365 | 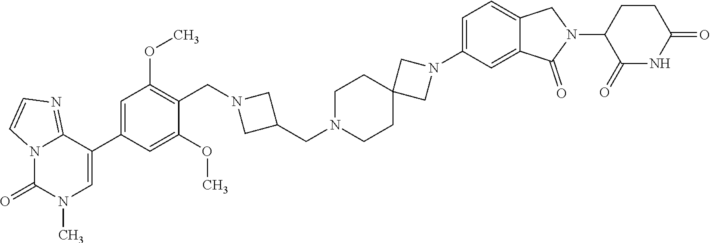 [SMILES: COc1cc(-c2cn(C)c(=O)n3ccnc23)cc(OC)c1CN1CC(CN2CCC3(CC2)CN(c2ccc4c(c2)C(=O)N(C2CCC(=O)NC2=O)C4)C3)C1] |
| D366 | 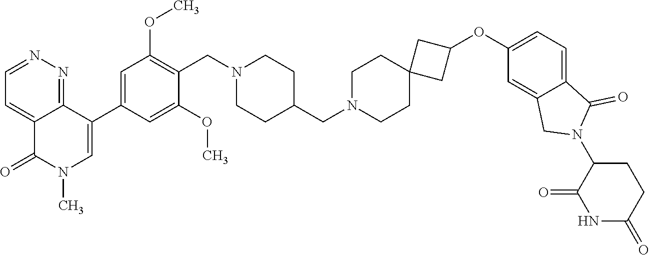 [SMILES: COc1cc(-c2cn(C)c(=O)c3ccnnc23)cc(OC)c1CN1CCC(CN2CCC3(CC2)CC(Oc2ccc4c(c2)CN(C2CCC(=O)NC2=O)C4=O)C3)CC1] |
| D367 | 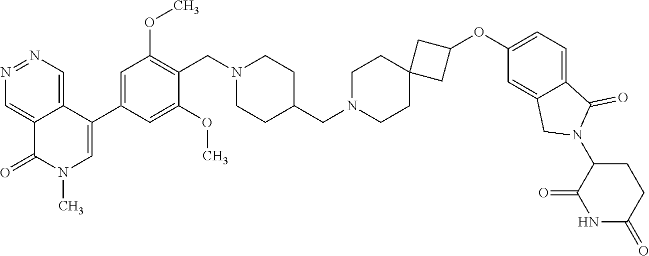 [SMILES: COc1cc(-c2cn(C)c(=O)c3cnncc23)cc(OC)c1CN1CCC(CN2CCC3(CC2)CC(Oc2ccc4c(c2)CN(C2CCC(=O)NC2=O)C4=O)C3)CC1] |
| D368 | 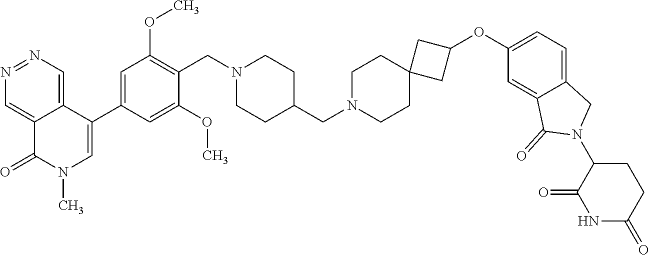 [SMILES: COc1cc(-c2cn(C)c(=O)c3cnncc23)cc(OC)c1CN1CCC(CN2CCC3(CC2)CC(Oc2ccc4c(c2)C(=O)N(C2CCC(=O)NC2=O)C4)C3)CC1] |
| D369 | 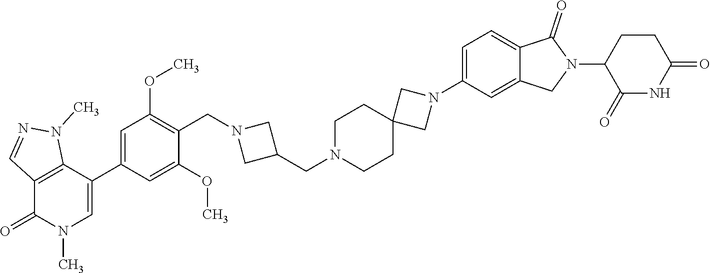 [SMILES: COc1cc(-c2cn(C)c(=O)c3cnn(C)c23)cc(OC)c1CN1CC(CN2CCC3(CC2)CN(c2ccc4c(c2)CN(C2CCC(=O)NC2=O)C4=O)C3)C1] |
| D370 | 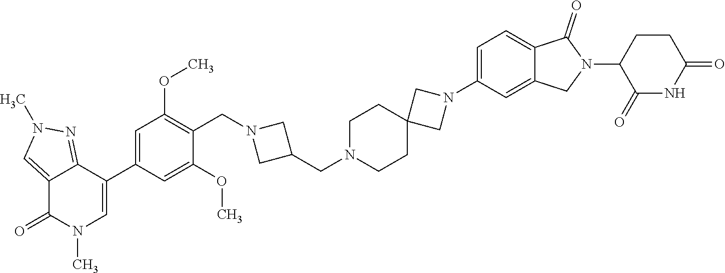 [SMILES: COc1cc(-c2cn(C)c(=O)c3cn(C)nc23)cc(OC)c1CN1CC(CN2CCC3(CC2)CN(c2ccc4c(c2)CN(C2CCC(=O)NC2=O)C4=O)C3)C1] |
| D371 | 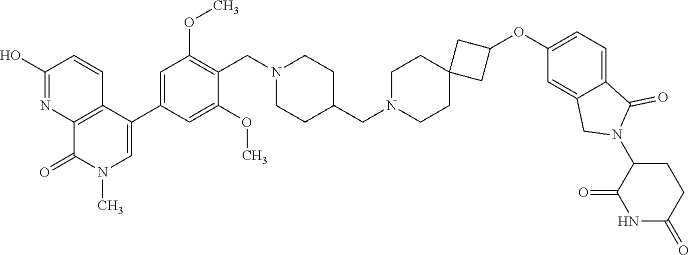 [SMILES: COc1cc(-c2cn(C)c(=O)c3nc(O)ccc23)cc(OC)c1CN1CCC(CN2CCC3(CC2)CC(Oc2ccc4c(c2)CN(C2CCC(=O)NC2=O)C4=O)C3)CC1] |

TABLE 1C
Compounds DD1-DD10 of the Disclosure

| Com- | |
|---|---|
| pound | |
| No. | Structure |
| DD1 | 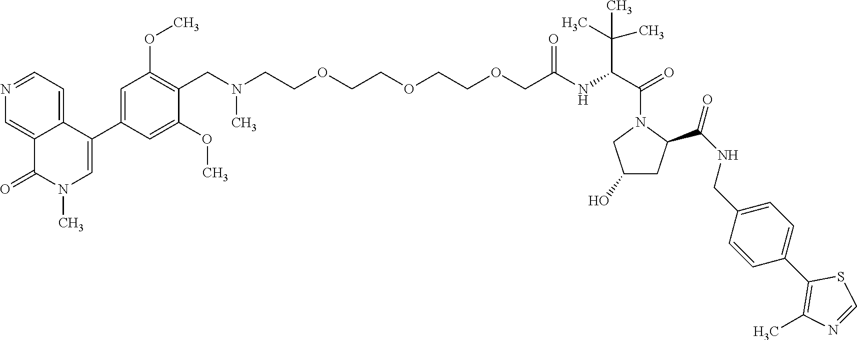 [SMILES: COc1cc(-c2cn(C)c(=O)c3cnccc23)cc(OC)c1CN(C)CCOCCOCCOCC(=O)N[C@@H](C(=O)N1C[C@@H](O)C[C@@H]1C(=O)NCc1ccc(-c2scnc2C)cc1)C(C)(C)C] |
| DD2 | 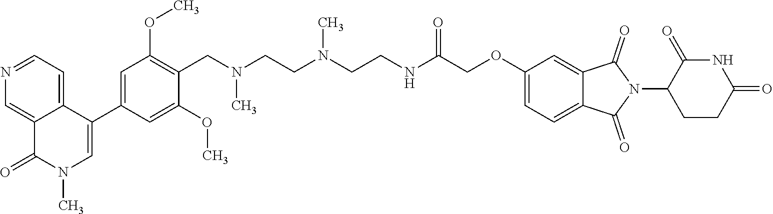 [SMILES: COc1cc(-c2cn(C)c(=O)c3cnccc23)cc(OC)c1CN(C)CCN(C)CCNC(=O)COc1ccc2c(c1)C(=O)N(C1CCC(=O)NC1=O)C2=O] |
| DD3 | 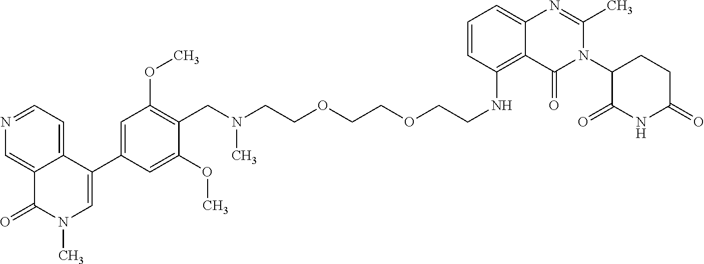 [SMILES: COc1cc(-c2cn(C)c(=O)c3cnccc23)cc(OC)c1CN(C)CCOCCOCCNc1cccc2nc(C)n(C3CCC(=O)NC3=O)c(=O)c12] |
| DD4 | 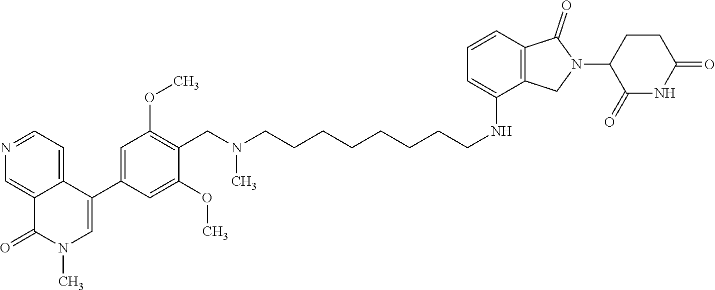 [SMILES: COc1cc(-c2cn(C)c(=O)c3cnccc23)cc(OC)c1CN(C)CCCCCCCCNc1cccc2c1CN(C1CCC(=O)NC1=O)C2=O] |
| DD5 | 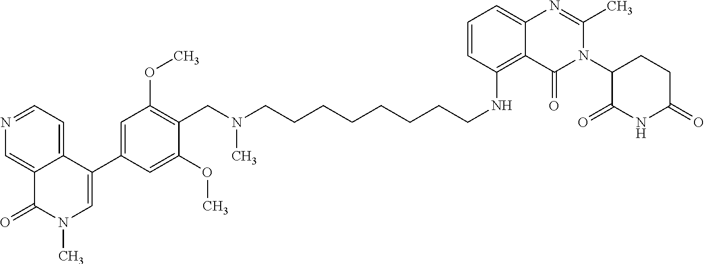 [SMILES: COc1cc(-c2cn(C)c(=O)c3cnccc23)cc(OC)c1CN(C)CCCCCCCCNc1cccc2nc(C)n(C3CCC(=O)NC3=O)c(=O)c12] |
| DD6 | 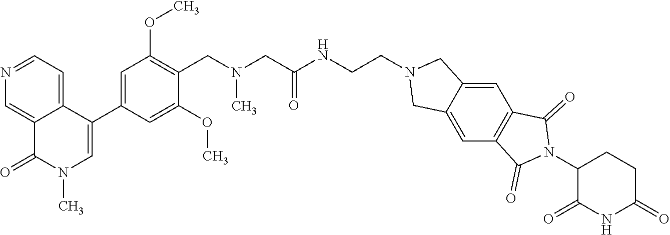 [SMILES: COc1cc(-c2cn(C)c(=O)c3cnccc23)cc(OC)c1CN(C)CC(=O)NCCN1Cc2cc3c(cc2C1)C(=O)N(C1CCC(=O)NC1=O)C3=O] |
| DD7 | 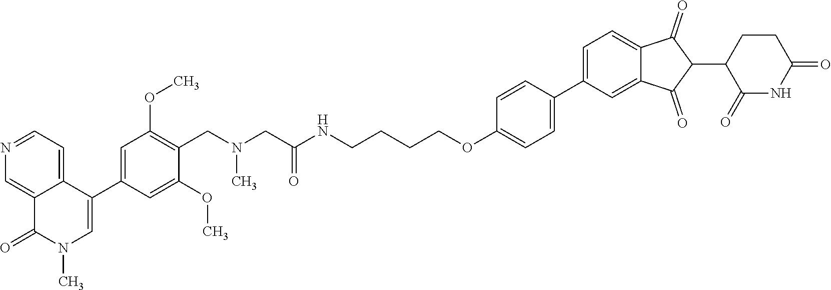 [SMILES: COc1cc(-c2cn(C)c(=O)c3cnccc23)cc(OC)c1CN(C)CC(=O)NCCCCOc1ccc(-c2ccc3c(c2)C(=O)C(C2CCC(=O)NC2=O)C3=O)cc1] |
| DD8 | 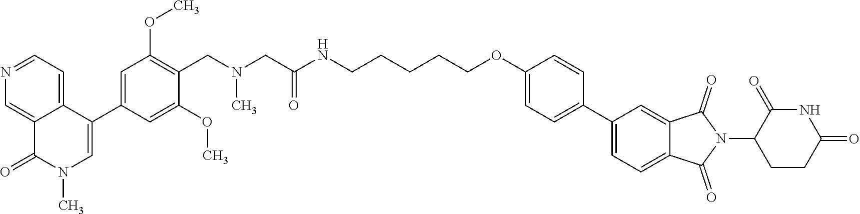 [SMILES: COc1cc(-c2cn(C)c(=O)c3cnccc23)cc(OC)c1CN(C)CC(=O)NCCCCCOc1ccc(-c2ccc3c(c2)C(=O)N(C2CCC(=O)NC2=O)C3=O)cc1] |
| DD9 | 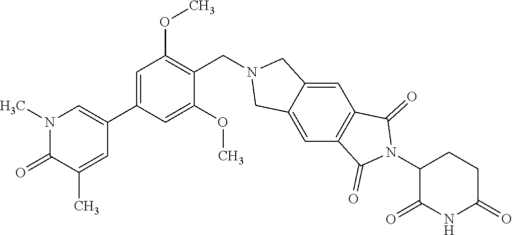 [SMILES: COc1cc(-c2cc(C)c(=O)n(C)c2)cc(OC)c1CN1Cc2cc3c(cc2C1)C(=O)N(C1CCC(=O)NC1=O)C3=O] |
| DD10 | 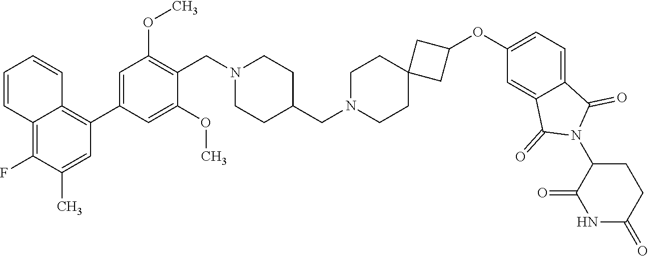 [SMILES: COc1cc(-c2cc(C)c(F)c3ccccc23)cc(OC)c1CN1CCC(CN2CCC3(CC2)CC(Oc2ccc4c(c2)C(=O)N(C2CCC(=O)NC2=O)C4=O)C3)CC1] |

TABLE 1D
Compounds D372-D477 of the disclosure

| Compound | |
|---|---|
| No. | Structure |
| D372 | 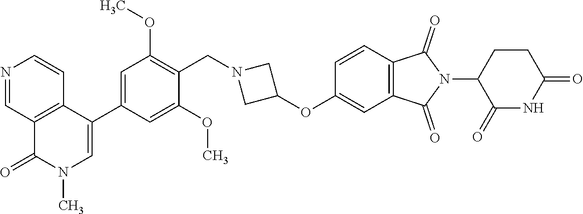 [SMILES: COc1cc(-c2cn(C)c(=O)c3cnccc23)cc(OC)c1CN1CC(Oc2ccc3c(c2)C(=O)N(C2CCC(=O)NC2=O)C3=O)C1] |
| D373 | 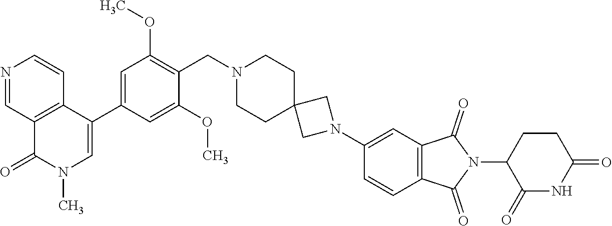 [SMILES: COc1cc(-c2cn(C)c(=O)c3cnccc23)cc(OC)c1CN1CCC2(CC1)CN(c1ccc3c(c1)C(=O)N(C1CCC(=O)NC1=O)C3=O)C2] |
| D374 | 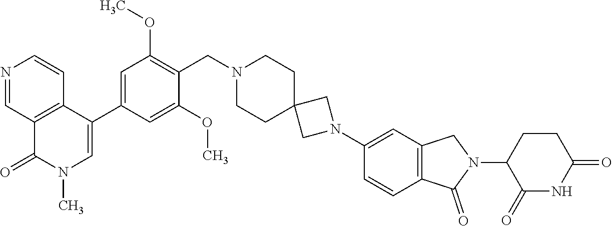 [SMILES: COc1cc(-c2cn(C)c(=O)c3cnccc23)cc(OC)c1CN1CCC2(CC1)CN(c1ccc3c(c1)CN(C1CCC(=O)NC1=O)C3=O)C2] |
| D375 | 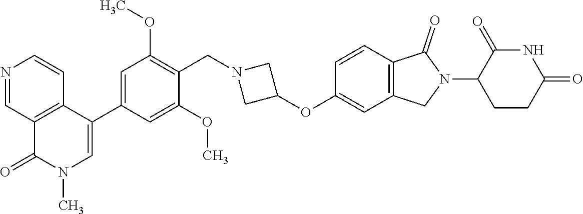 [SMILES: COc1cc(-c2cn(C)c(=O)c3cnccc23)cc(OC)c1CN1CC(Oc2ccc3c(c2)CN(C2CCC(=O)NC2=O)C3=O)C1] |
| D376 | 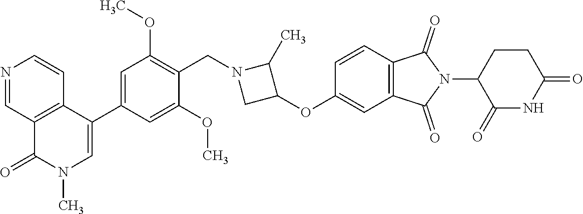 [SMILES: COc1cc(-c2cn(C)c(=O)c3cnccc23)cc(OC)c1CN1CC(Oc2ccc3c(c2)C(=O)N(C2CCC(=O)NC2=O)C3=O)C1C] |
| D377 | 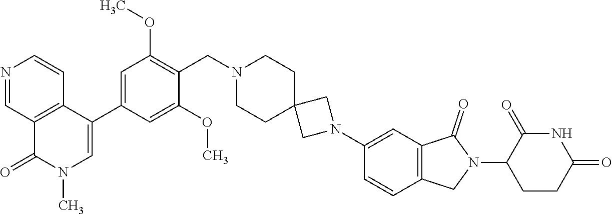 [SMILES: COc1cc(-c2cn(C)c(=O)c3cnccc23)cc(OC)c1CN1CCC2(CC1)CN(c1ccc3c(c1)C(=O)N(C1CCC(=O)NC1=O)C3)C2] |
| D378 | 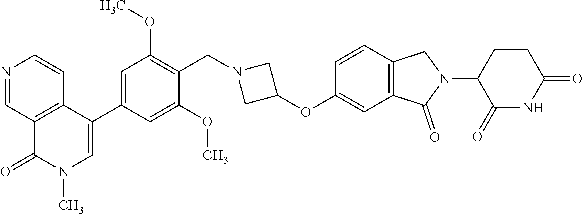 [SMILES: COc1cc(-c2cn(C)c(=O)c3cnccc23)cc(OC)c1CN1CC(Oc2ccc3c(c2)C(=O)N(C2CCC(=O)NC2=O)C3)C1] |
| D379 | 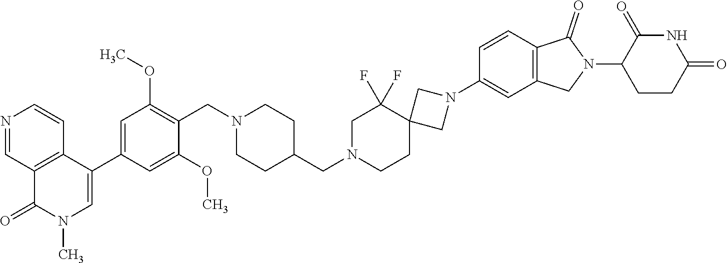 [SMILES: COc1cc(-c2cn(C)c(=O)c3cnccc23)cc(OC)c1CN1CCC(CN2CCC3(CN(c4ccc5c(c4)CN(C4CCC(=O)NC4=O)C5=O)C3)C(F)(F)C2)CC1] |
| D380 | 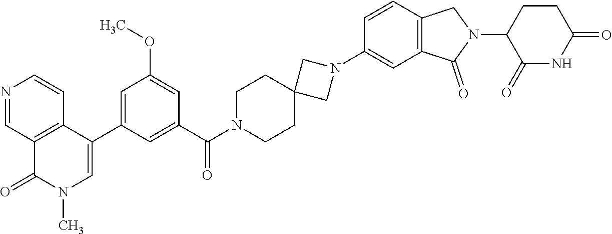 [SMILES: COc1cc(C(=O)N2CCC3(CC2)CN(c2ccc4c(c2)C(=O)N(C2CCC(=O)NC2=O)C4)C3)cc(-c2cn(C)c(=O)c3cnccc23)c1] |
| D381 | 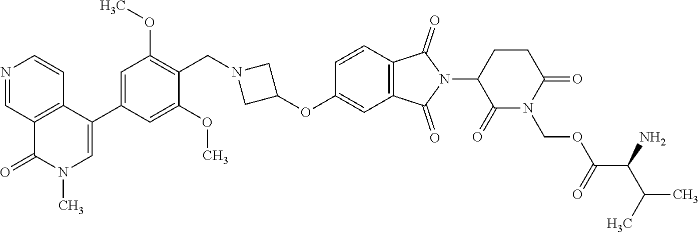 [SMILES: COc1cc(-c2cn(C)c(=O)c3cnccc23)cc(OC)c1CN1CC(Oc2ccc3c(c2)C(=O)N(C2CCC(=O)N(COC(=O)[C@@H](N)C(C)C)C2=O)C3=O)C1] |
| D382 | 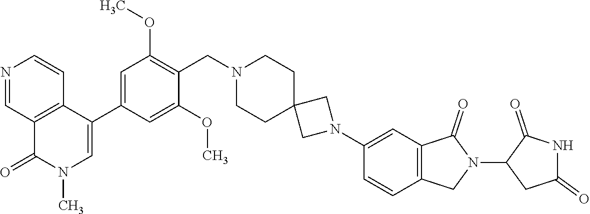 [SMILES: COc1cc(-c2cn(C)c(=O)c3cnccc23)cc(OC)c1CN1CCC2(CC1)CN(c1ccc3c(c1)C(=O)N(C1CC(=O)NC1=O)C3)C2] |
| D383 | 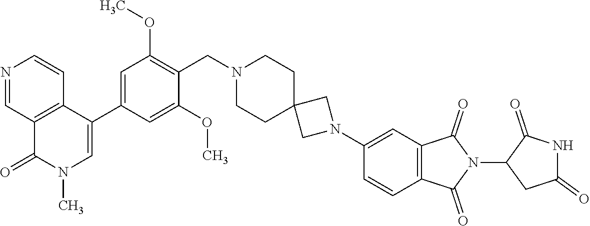 [SMILES: COc1cc(-c2cn(C)c(=O)c3cnccc23)cc(OC)c1CN1CCC2(CC1)CN(c1ccc3c(c1)C(=O)N(C1CC(=O)NC1=O)C3=O)C2] |
| D384 | 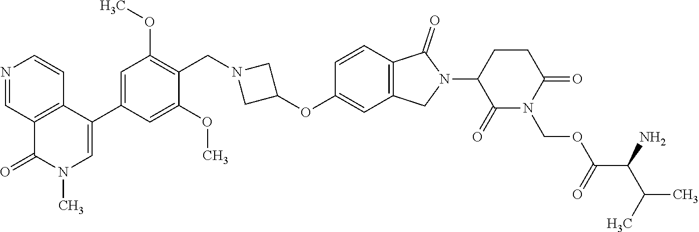 [SMILES: COc1cc(-c2cn(C)c(=O)c3cnccc23)cc(OC)c1CN1CC(Oc2ccc3c(c2)CN(C2CCC(=O)N(COC(=O)[C@@H](N)C(C)C)C2=O)C3=O)C1] |
| D385 | 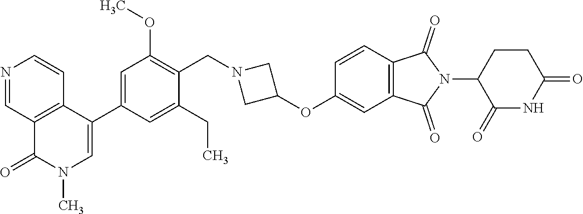 [SMILES: CCc1cc(-c2cn(C)c(=O)c3cnccc23)cc(OC)c1CN1CC(Oc2ccc3c(c2)C(=O)N(C2CCC(=O)NC2=O)C3=O)C1] |
| D386 | 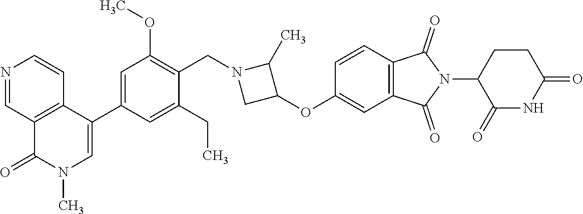 [SMILES: CCc1cc(-c2cn(C)c(=O)c3cnccc23)cc(OC)c1CN1CC(Oc2ccc3c(c2)C(=O)N(C2CCC(=O)NC2=O)C3=O)C1C] |
| D387 | 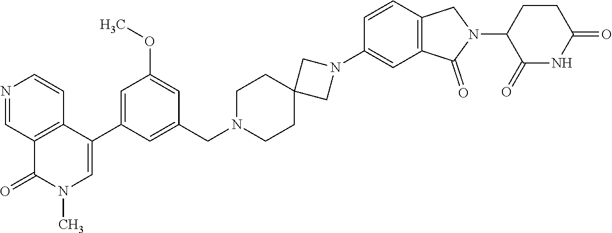 [SMILES: COc1cc(CN2CCC3(CC2)CN(c2ccc4c(c2)C(=O)N(C2CCC(=O)NC2=O)C4)C3)cc(-c2cn(C)c(=O)c3cnccc23)c1] |
| D388 | 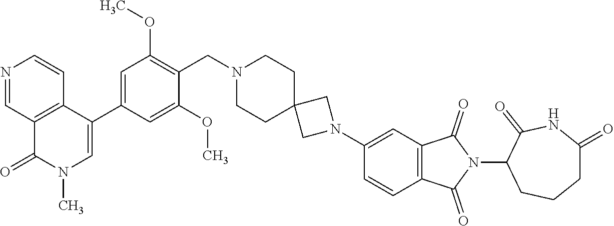 [SMILES: COc1cc(-c2cn(C)c(=O)c3cnccc23)cc(OC)c1CN1CCC2(CC1)CN(c1ccc3c(c1)C(=O)N(C1CCCC(=O)NC1=O)C3=O)C2] |
| D389 | 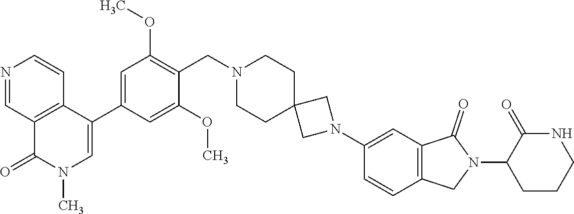 [SMILES: COc1cc(-c2cn(C)c(=O)c3cnccc23)cc(OC)c1CN1CCC2(CC1)CN(c1ccc3c(c1)C(=O)N(C1CCCNC1=O)C3)C2] |
| D390 | 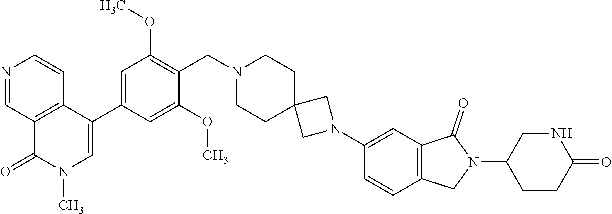 [SMILES: COc1cc(-c2cn(C)c(=O)c3cnccc23)cc(OC)c1CN1CCC2(CC1)CN(c1ccc3c(c1)C(=O)N(C1CCC(=O)NC1)C3)C2] |
| D391 | 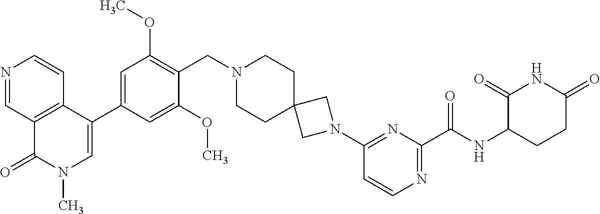 [SMILES: COc1cc(-c2cn(C)c(=O)c3cnccc23)cc(OC)c1CN1CCC2(CC1)CN(c1ccnc(C(=O)NC3CCC(=O)NC3=O)n1)C2] |
| D392 | 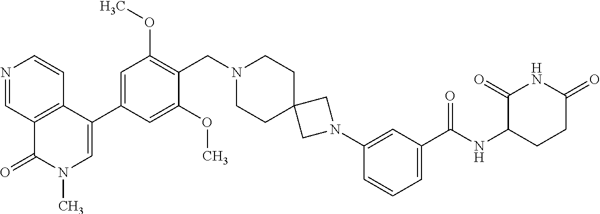 [SMILES: COc1cc(-c2cn(C)c(=O)c3cnccc23)cc(OC)c1CN1CCC2(CC1)CN(c1cccc(C(=O)NC3CCC(=O)NC3=O)c1)C2] |
| D393 | 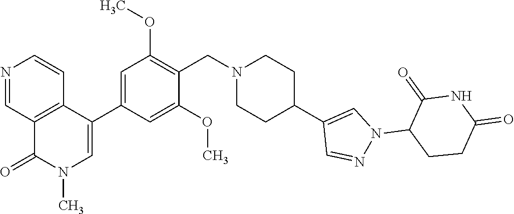 [SMILES: COc1cc(-c2cn(C)c(=O)c3cnccc23)cc(OC)c1CN1CCC(c2cnn(C3CCC(=O)NC3=O)c2)CC1] |
| D394 | 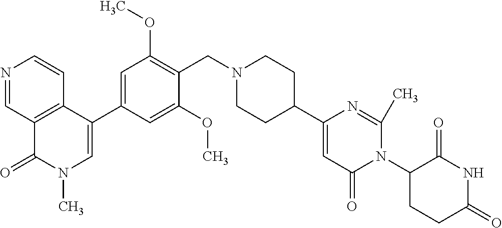 [SMILES: COc1cc(-c2cn(C)c(=O)c3cnccc23)cc(OC)c1CN1CCC(c2cc(=O)n(C3CCC(=O)NC3=O)c(C)n2)CC1] |
| D395 | 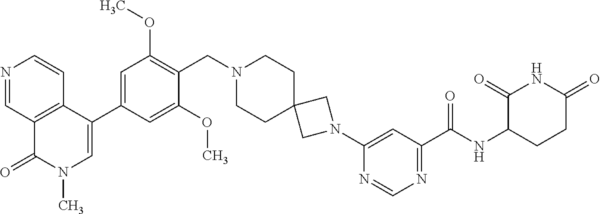 [SMILES: COc1cc(-c2cn(C)c(=O)c3cnccc23)cc(OC)c1CN1CCC2(CC1)CN(c1cc(C(=O)NC3CCC(=O)NC3=O)ncn1)C2] |
| D396 | 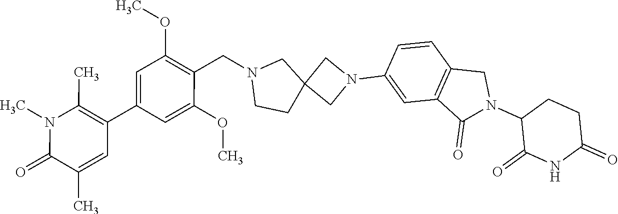 [SMILES: COc1cc(-c2cc(C)c(=O)n(C)c2C)cc(OC)c1CN1CCC2(C1)CN(c1ccc3c(c1)C(=O)N(C1CCC(=O)NC1=O)C3)C2] |
| D397 | 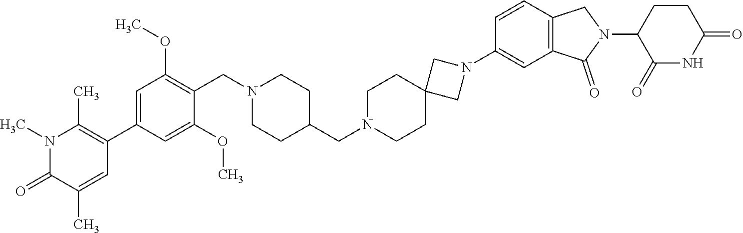 [SMILES: COc1cc(-c2cc(C)c(=O)n(C)c2C)cc(OC)c1CN1CCC(CN2CCC3(CC2)CN(c2ccc4c(c2)C(=O)N(C2CCC(=O)NC2=O)C4)C3)CC1] |
| D398 | 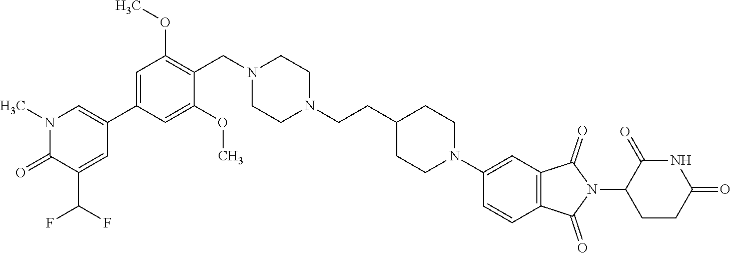 [SMILES: COc1cc(-c2cc(C(F)F)c(=O)n(C)c2)cc(OC)c1CN1CCN(CCC2CCN(c3ccc4c(c3)C(=O)N(C3CCC(=O)NC3=O)C4=O)CC2)CC1] |
| D399 | 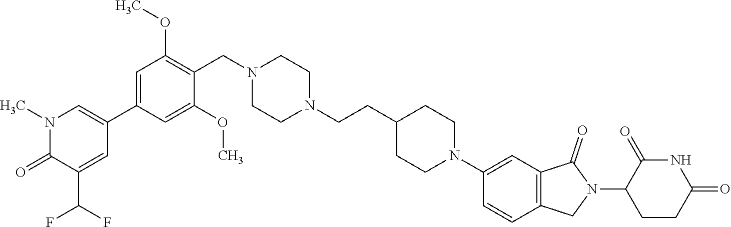 [SMILES: COc1cc(-c2cc(C(F)F)c(=O)n(C)c2)cc(OC)c1CN1CCN(CCC2CCN(c3ccc4c(c3)C(=O)N(C3CCC(=O)NC3=O)C4)CC2)CC1] |
| D400 | 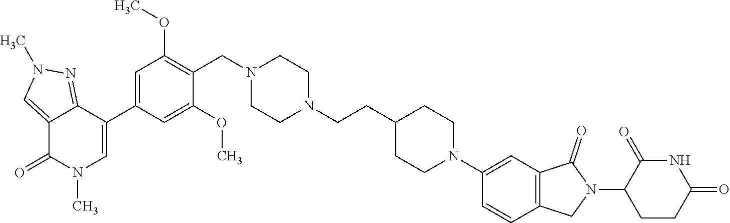 [SMILES: COc1cc(-c2cn(C)c(=O)c3cn(C)nc23)cc(OC)c1CN1CCN(CCC2CCN(c3ccc4c(c3)C(=O)N(C3CCC(=O)NC3=O)C4)CC2)CC1] |
| D401 | 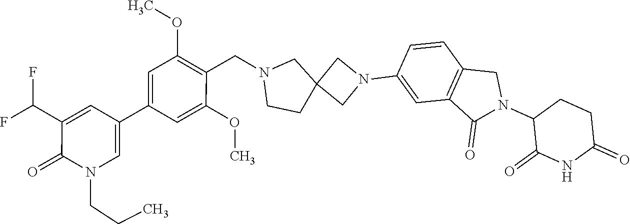 [SMILES: CCCn1cc(-c2cc(OC)c(CN3CCC4(C3)CN(c3ccc5c(c3)C(=O)N(C3CCC(=O)NC3=O)C5)C4)c(OC)c2)cc(C(F)F)c1=O] |
| D402 | 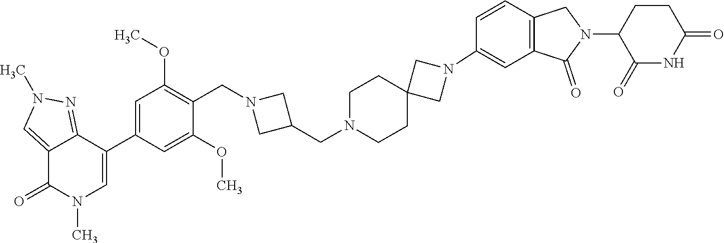 [SMILES: COc1cc(-c2cn(C)c(=O)c3cn(C)nc23)cc(OC)c1CN1CC(CN2CCC3(CC2)CN(c2ccc4c(c2)C(=O)N(C2CCC(=O)NC2=O)C4)C3)C1] |
| D403 | 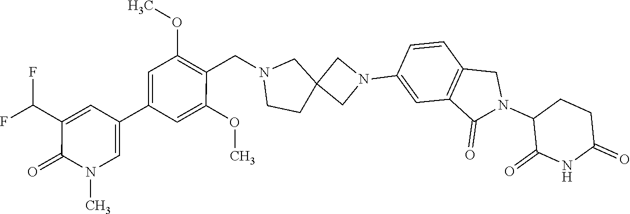 [SMILES: COc1cc(-c2cc(C(F)F)c(=O)n(C)c2)cc(OC)c1CN1CCC2(C1)CN(c1ccc3c(c1)C(=O)N(C1CCC(=O)NC1=O)C3)C2] |
| D404 | 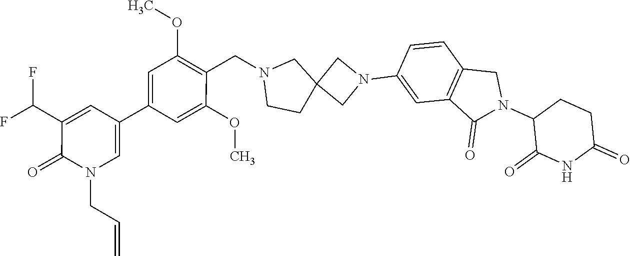 [SMILES: C=CCn1cc(-c2cc(OC)c(CN3CCC4(C3)CN(c3ccc5c(c3)C(=O)N(C3CCC(=O)NC3=O)C5)C4)c(OC)c2)cc(C(F)F)c1=O] |
| D405 | 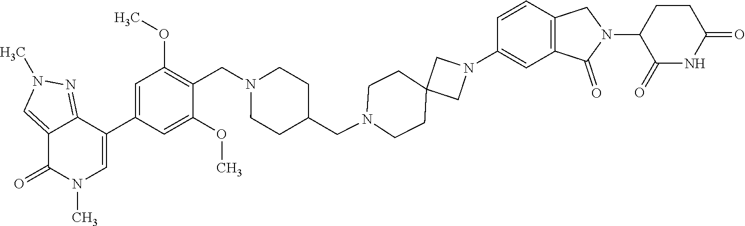 [SMILES: COc1cc(-c2cn(C)c(=O)c3cn(C)nc23)cc(OC)c1CN1CCC(CN2CCC3(CC2)CN(c2ccc4c(c2)C(=O)N(C2CCC(=O)NC2=O)C4)C3)CC1] |
| D406 | 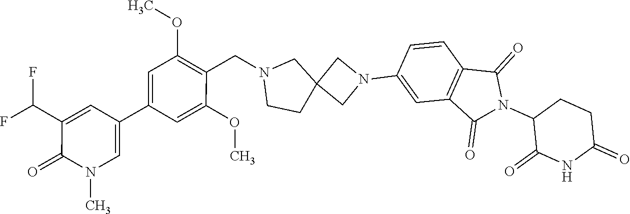 [SMILES: COc1cc(-c2cc(C(F)F)c(=O)n(C)c2)cc(OC)c1CN1CCC2(C1)CN(c1ccc3c(c1)C(=O)N(C1CCC(=O)NC1=O)C3=O)C2] |
| D407 | 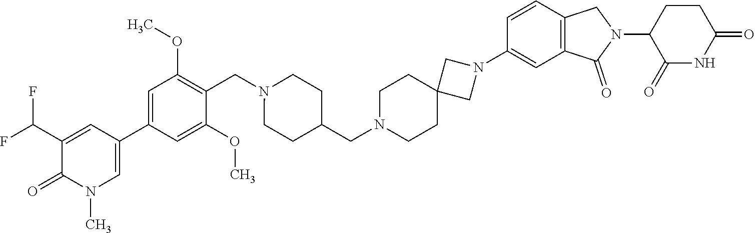 [SMILES: COc1cc(-c2cc(C(F)F)c(=O)n(C)c2)cc(OC)c1CN1CCC(CN2CCC3(CC2)CN(c2ccc4c(c2)C(=O)N(C2CCC(=O)NC2=O)C4)C3)CC1] |
| D408 | 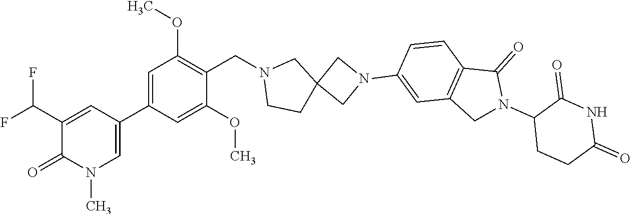 [SMILES: COc1cc(-c2cc(C(F)F)c(=O)n(C)c2)cc(OC)c1CN1CCC2(C1)CN(c1ccc3c(c1)CN(C1CCC(=O)NC1=O)C3=O)C2] |
| D409 | 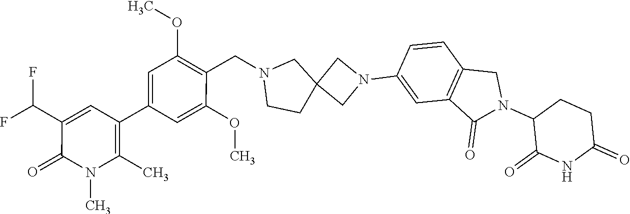 [SMILES: COc1cc(-c2cc(C(F)F)c(=O)n(C)c2C)cc(OC)c1CN1CCC2(C1)CN(c1ccc3c(c1)C(=O)N(C1CCC(=O)NC1=O)C3)C2] |
| D410 | 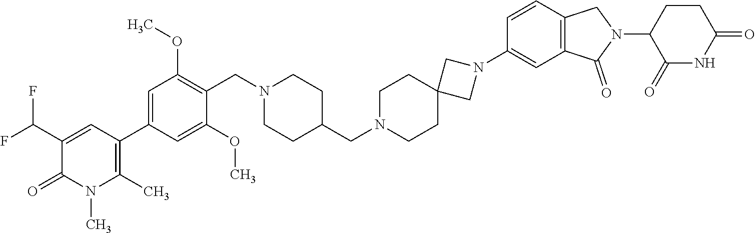 [SMILES: COc1cc(-c2cc(C(F)F)c(=O)n(C)c2C)cc(OC)c1CN1CCC(CN2CCC3(CC2)CN(c2ccc4c(c2)C(=O)N(C2CCC(=O)NC2=O)C4)C3)CC1] |
| D411 | 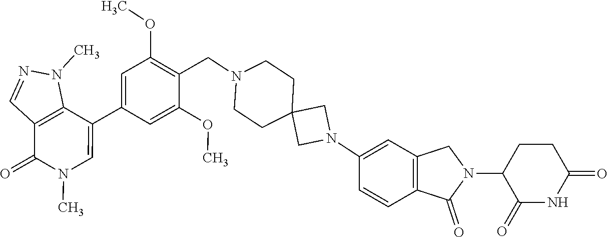 [SMILES: COc1cc(-c2cn(C)c(=O)c3cnn(C)c23)cc(OC)c1CN1CCC2(CC1)CN(c1ccc3c(c1)CN(C1CCC(=O)NC1=O)C3=O)C2] |
| D412 | 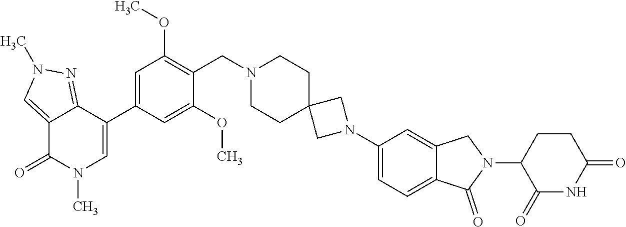 [SMILES: COc1cc(-c2cn(C)c(=O)c3cn(C)nc23)cc(OC)c1CN1CCC2(CC1)CN(c1ccc3c(c1)CN(C1CCC(=O)NC1=O)C3=O)C2] |
| D413 | 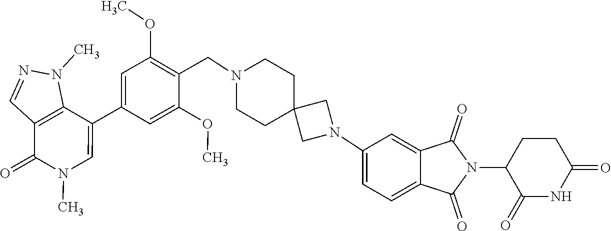 [SMILES: COc1cc(-c2cn(C)c(=O)c3cnn(C)c23)cc(OC)c1CN1CCC2(CC1)CN(c1ccc3c(c1)C(=O)N(C1CCC(=O)NC1=O)C3=O)C2] |
| D414 | 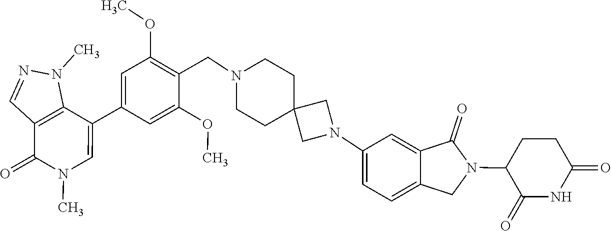 [SMILES: COc1cc(-c2cn(C)c(=O)c3cnn(C)c23)cc(OC)c1CN1CCC2(CC1)CN(c1ccc3c(c1)C(=O)N(C1CCC(=O)NC1=O)C3)C2] |
| D415 | 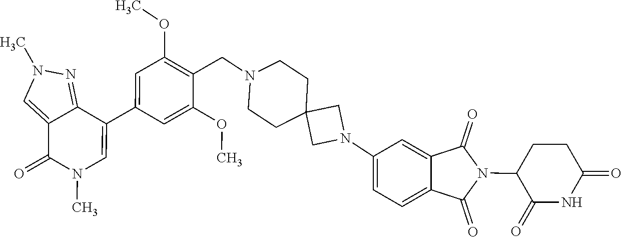 [SMILES: COc1cc(-c2cn(C)c(=O)c3cn(C)nc23)cc(OC)c1CN1CCC2(CC1)CN(c1ccc3c(c1)C(=O)N(C1CCC(=O)NC1=O)C3=O)C2] |
| D416 | 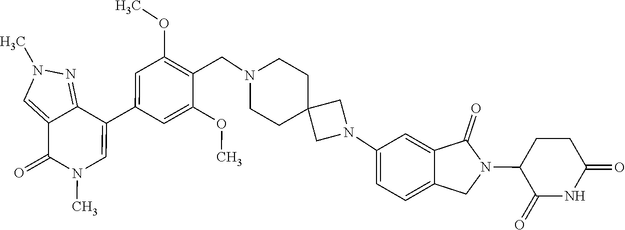 [SMILES: COc1cc(-c2cn(C)c(=O)c3cn(C)nc23)cc(OC)c1CN1CCC2(CC1)CN(c1ccc3c(c1)C(=O)N(C1CCC(=O)NC1=O)C3)C2] |
| D417 | 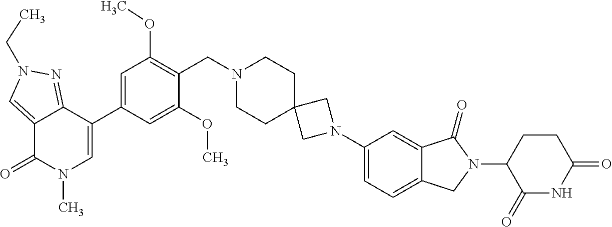 [SMILES: CCn1cc2c(=O)n(C)cc(-c3cc(OC)c(CN4CCC5(CC4)CN(c4ccc6c(c4)C(=O)N(C4CCC(=O)NC4=O)C6)C5)c(OC)c3)c2n1] |
| D418 | 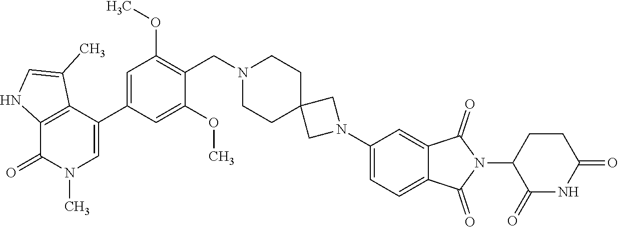 [SMILES: COc1cc(-c2cn(C)c(=O)c3[nH]cc(C)c23)cc(OC)c1CN1CCC2(CC1)CN(c1ccc3c(c1)C(=O)N(C1CCC(=O)NC1=O)C3=O)C2] |
| D419 | 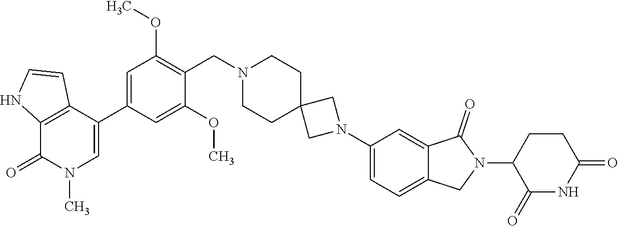 [SMILES: COc1cc(-c2cn(C)c(=O)c3[nH]ccc23)cc(OC)c1CN1CCC2(CC1)CN(c1ccc3c(c1)C(=O)N(C1CCC(=O)NC1=O)C3)C2] |
| D420 | 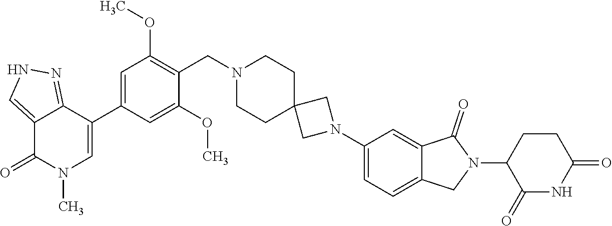 [SMILES: COc1cc(-c2cn(C)c(=O)c3c[nH]nc23)cc(OC)c1CN1CCC2(CC1)CN(c1ccc3c(c1)C(=O)N(C1CCC(=O)NC1=O)C3)C2] |
| D421 | 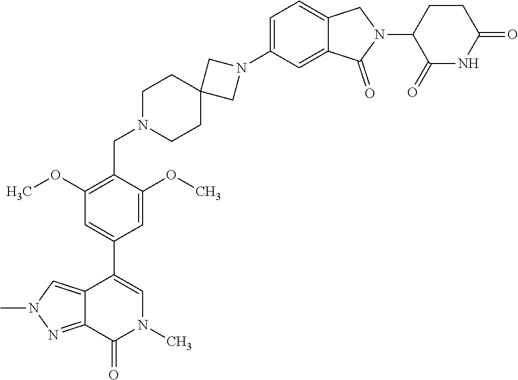 [SMILES: COc1cc(-c2cn(C)c(=O)c3nn(C)cc23)cc(OC)c1CN1CCC2(CC1)CN(c1ccc3c(c1)C(=O)N(C1CCC(=O)NC1=O)C3)C2] |
| D422 | 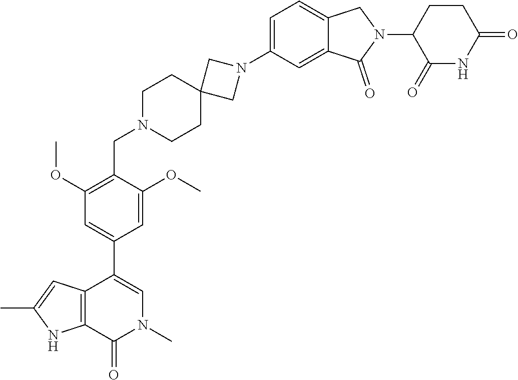 [SMILES: COc1cc(-c2cn(C)c(=O)c3[nH]c(C)cc23)cc(OC)c1CN1CCC2(CC1)CN(c1ccc3c(c1)C(=O)N(C1CCC(=O)NC1=O)C3)C2] |
| D423 | 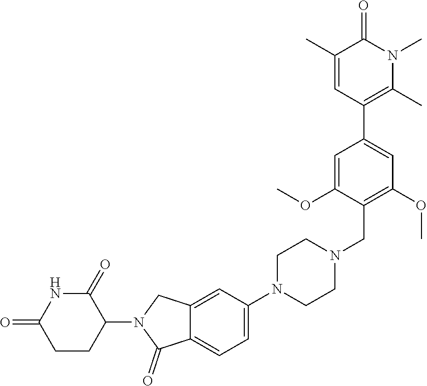 [SMILES: COc1cc(-c2cc(C)c(=O)n(C)c2C)cc(OC)c1CN1CCN(c2ccc3c(c2)CN(C2CCC(=O)NC2=O)C3=O)CC1] |
| D424 | 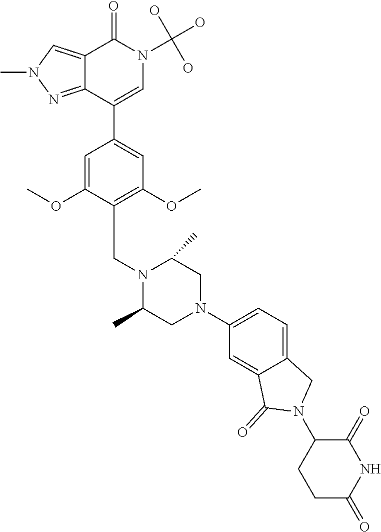 [SMILES: COc1cc(-c2cn(C(O)(O)O)c(=O)c3cn(C)nc23)cc(OC)c1CN1[C@H](C)CN(c2ccc3c(c2)C(=O)N(C2CCC(=O)NC2=O)C3)C[C@H]1C] |
| D425 | 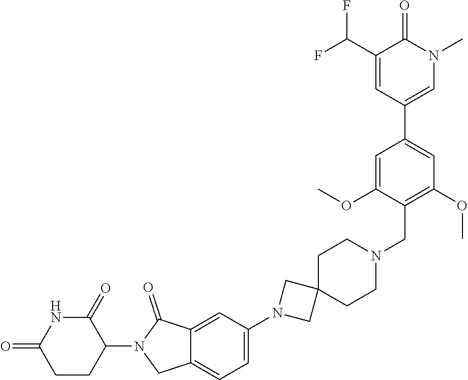 [SMILES: COc1cc(-c2cc(C(F)F)c(=O)n(C)c2)cc(OC)c1CN1CCC2(CC1)CN(c1ccc3c(c1)C(=O)N(C1CCC(=O)NC1=O)C3)C2] |
| D426 | 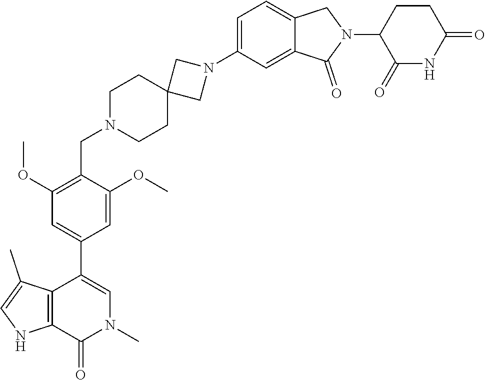 [SMILES: COc1cc(-c2cn(C)c(=O)c3[nH]cc(C)c23)cc(OC)c1CN1CCC2(CC1)CN(c1ccc3c(c1)C(=O)N(C1CCC(=O)NC1=O)C3)C2] |
| D427 | 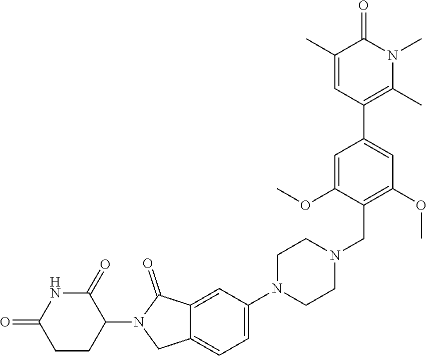 [SMILES: COc1cc(-c2cc(C)c(=O)n(C)c2C)cc(OC)c1CN1CCN(c2ccc3c(c2)C(=O)N(C2CCC(=O)NC2=O)C3)CC1] |
| D428 | 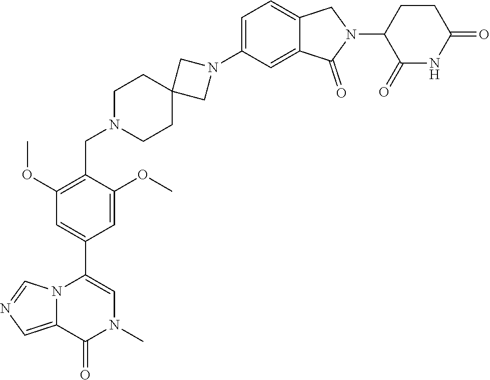 [SMILES: COc1cc(-c2cn(C)c(=O)c3cncn23)cc(OC)c1CN1CCC2(CC1)CN(c1ccc3c(c1)C(=O)N(C1CCC(=O)NC1=O)C3)C2] |
| D429 | 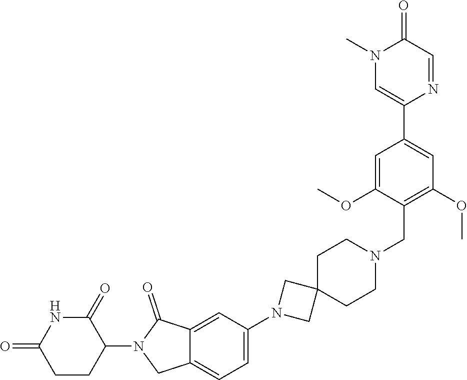 [SMILES: COc1cc(-c2cn(C)c(=O)cn2)cc(OC)c1CN1CCC2(CC1)CN(c1ccc3c(c1)C(=O)N(C1CCC(=O)NC1=O)C3)C2] |
| D430 | 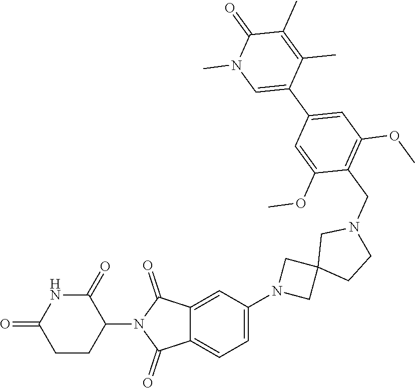 [SMILES: COc1cc(-c2cn(C)c(=O)c(C)c2C)cc(OC)c1CN1CCC2(C1)CN(c1ccc3c(c1)C(=O)N(C1CCC(=O)NC1=O)C3=O)C2] |
| D431 | 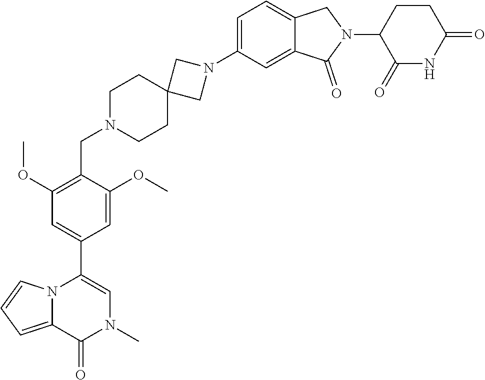 [SMILES: COc1cc(-c2cn(C)c(=O)c3cccn23)cc(OC)c1CN1CCC2(CC1)CN(c1ccc3c(c1)C(=O)N(C1CCC(=O)NC1=O)C3)C2] |
| D432 | 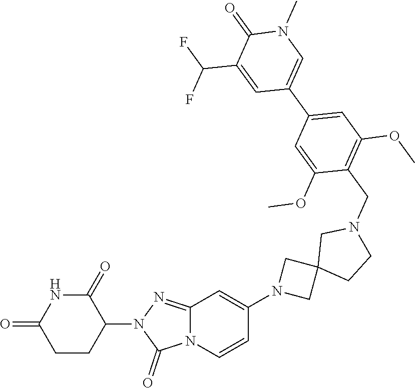 [SMILES: COc1cc(-c2cc(C(F)F)c(=O)n(C)c2)cc(OC)c1CN1CCC2(C1)CN(c1ccn3c(=O)n(C4CCC(=O)NC4=O)nc3c1)C2] |
| D433 | 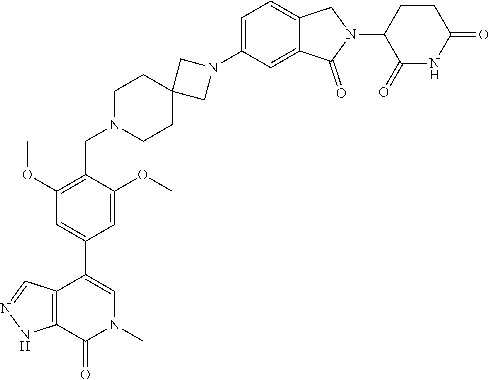 [SMILES: COc1cc(-c2cn(C)c(=O)c3[nH]ncc23)cc(OC)c1CN1CCC2(CC1)CN(c1ccc3c(c1)C(=O)N(C1CCC(=O)NC1=O)C3)C2] |
| D434 | 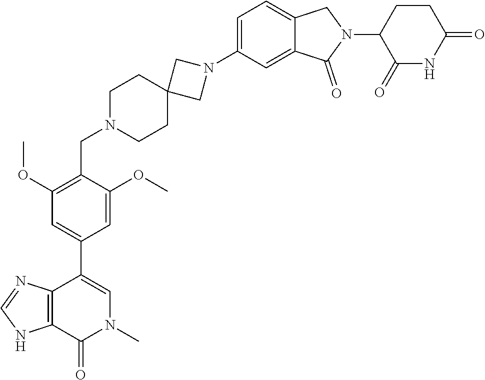 [SMILES: COc1cc(-c2cn(C)c(=O)c3[nH]cnc23)cc(OC)c1CN1CCC2(CC1)CN(c1ccc3c(c1)C(=O)N(C1CCC(=O)NC1=O)C3)C2] |
| D435 | 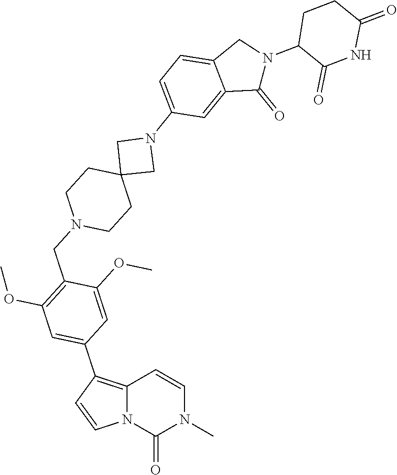 [SMILES: COc1cc(-c2ccn3c(=O)n(C)ccc23)cc(OC)c1CN1CCC2(CC1)CN(c1ccc3c(c1)C(=O)N(C1CCC(=O)NC1=O)C3)C2] |
| D436 | 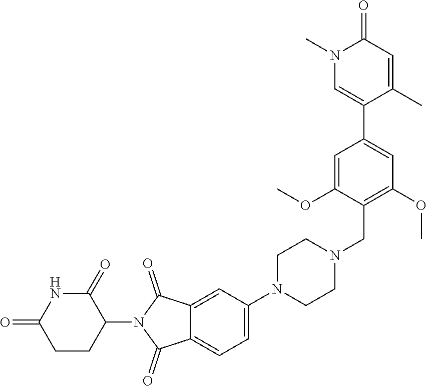 [SMILES: COc1cc(-c2cn(C)c(=O)cc2C)cc(OC)c1CN1CCN(c2ccc3c(c2)C(=O)N(C2CCC(=O)NC2=O)C3=O)CC1] |
| D437 | 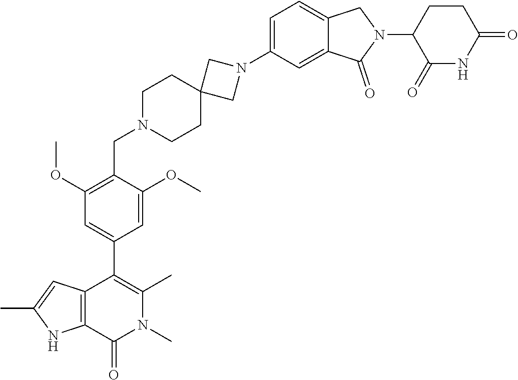 [SMILES: COc1cc(-c2c(C)n(C)c(=O)c3[nH]c(C)cc23)cc(OC)c1CN1CCC2(CC1)CN(c1ccc3c(c1)C(=O)N(C1CCC(=O)NC1=O)C3)C2] |
| D438 | 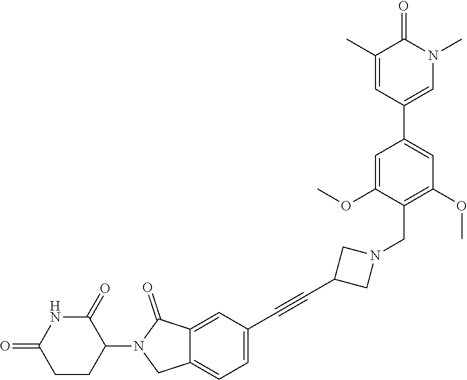 [SMILES: COc1cc(-c2cc(C)c(=O)n(C)c2)cc(OC)c1CN1CC(C#Cc2ccc3c(c2)C(=O)N(C2CCC(=O)NC2=O)C3)C1] |
| D439 | 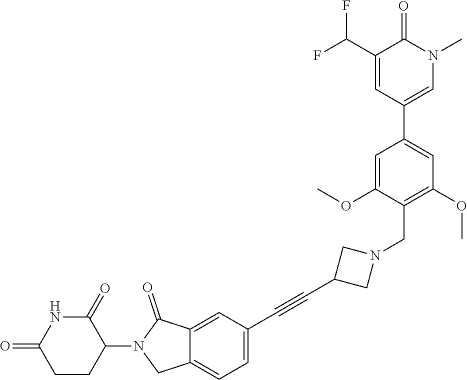 [SMILES: COc1cc(-c2cc(C(F)F)c(=O)n(C)c2)cc(OC)c1CN1CC(C#Cc2ccc3c(c2)C(=O)N(C2CCC(=O)NC2=O)C3)C1] |
| D440 | 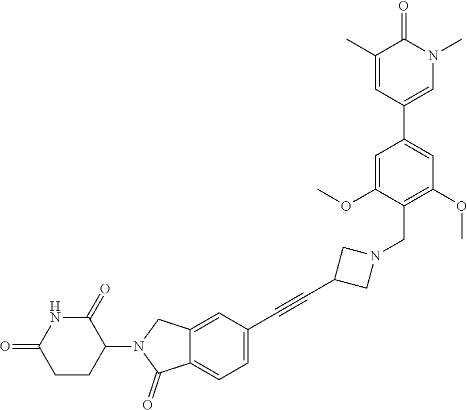 [SMILES: COc1cc(-c2cc(C)c(=O)n(C)c2)cc(OC)c1CN1CC(C#Cc2ccc3c(c2)CN(C2CCC(=O)NC2=O)C3=O)C1] |
| D441 | 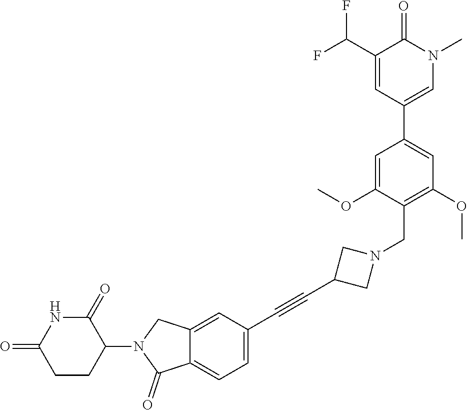 [SMILES: COc1cc(-c2cc(C(F)F)c(=O)n(C)c2)cc(OC)c1CN1CC(C#Cc2ccc3c(c2)CN(C2CCC(=O)NC2=O)C3=O)C1] |
| D442 | 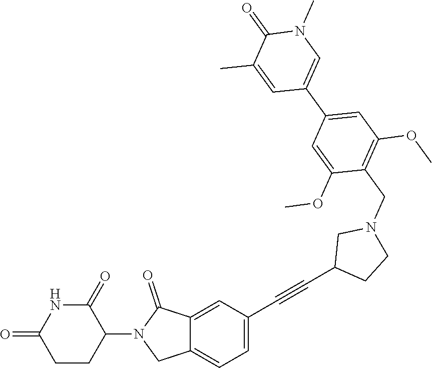 [SMILES: COc1cc(-c2cc(C)c(=O)n(C)c2)cc(OC)c1CN1CCC(C#Cc2ccc3c(c2)C(=O)N(C2CCC(=O)NC2=O)C3)C1] |
| D443 | 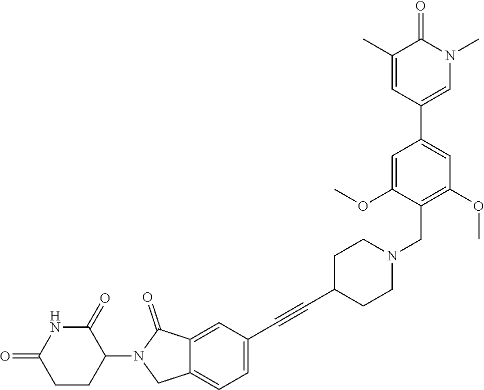 [SMILES: COc1cc(-c2cc(C)c(=O)n(C)c2)cc(OC)c1CN1CCC(C#Cc2ccc3c(c2)C(=O)N(C2CCC(=O)NC2=O)C3)CC1] |
| D444 | 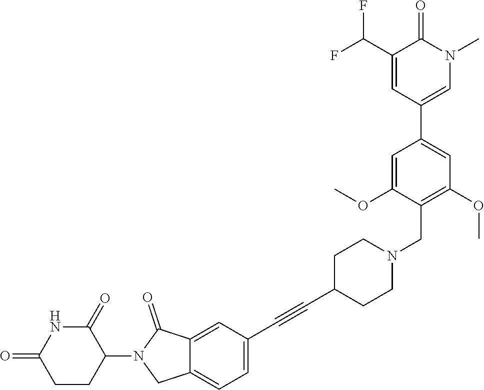 [SMILES: COc1cc(-c2cc(C(F)F)c(=O)n(C)c2)cc(OC)c1CN1CCC(C#Cc2ccc3c(c2)C(=O)N(C2CCC(=O)NC2=O)C3)CC1] |
| D445 | 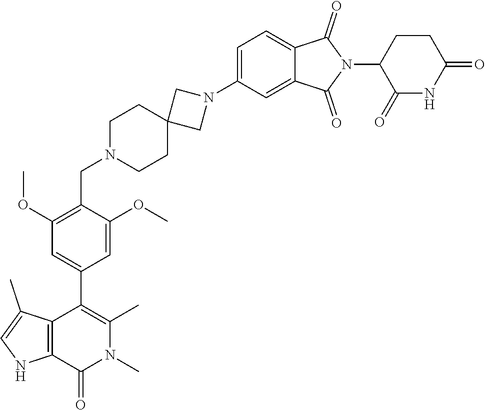 [SMILES: COc1cc(-c2c(C)n(C)c(=O)c3[nH]cc(C)c23)cc(OC)c1CN1CCC2(CC1)CN(c1ccc3c(c1)C(=O)N(C1CCC(=O)NC1=O)C3=O)C2] |
| D446 | 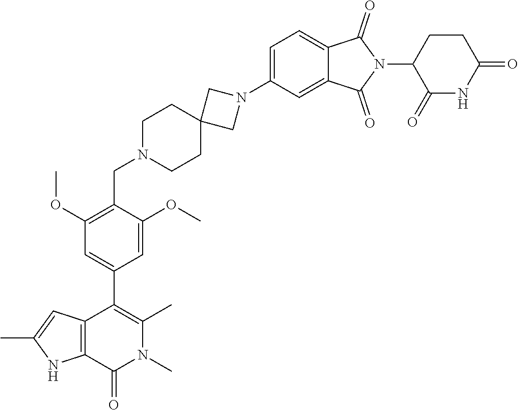 [SMILES: COc1cc(-c2c(C)n(C)c(=O)c3[nH]c(C)cc23)cc(OC)c1CN1CCC2(CC1)CN(c1ccc3c(c1)C(=O)N(C1CCC(=O)NC1=O)C3=O)C2] |
| D447 | 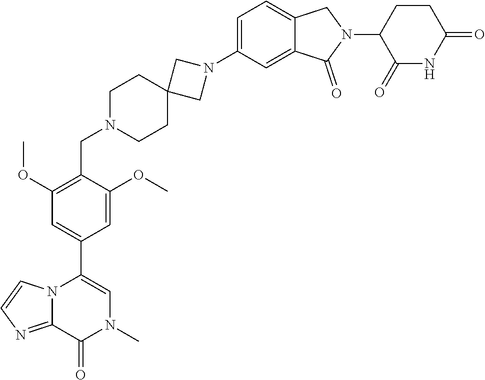 [SMILES: COc1cc(-c2cn(C)c(=O)c3nccn23)cc(OC)c1CN1CCC2(CC1)CN(c1ccc3c(c1)C(=O)N(C1CCC(=O)NC1=O)C3)C2] |
| D448 | 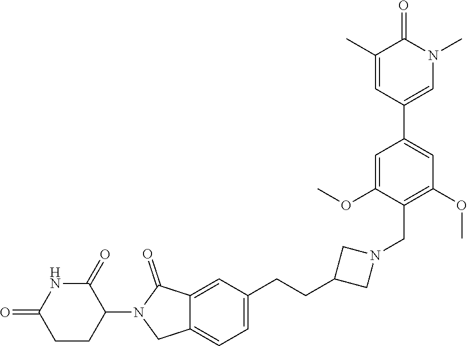 [SMILES: COc1cc(-c2cc(C)c(=O)n(C)c2)cc(OC)c1CN1CC(CCc2ccc3c(c2)C(=O)N(C2CCC(=O)NC2=O)C3)C1] |
| D449 | 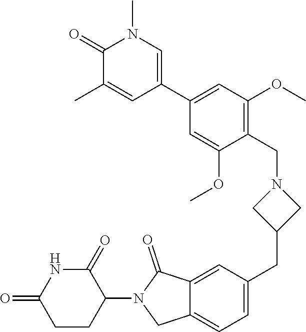 [SMILES: COc1cc(-c2cc(C)c(=O)n(C)c2)cc(OC)c1CN1CC(Cc2ccc3c(c2)C(=O)N(C2CCC(=O)NC2=O)C3)C1] |
| D450 | 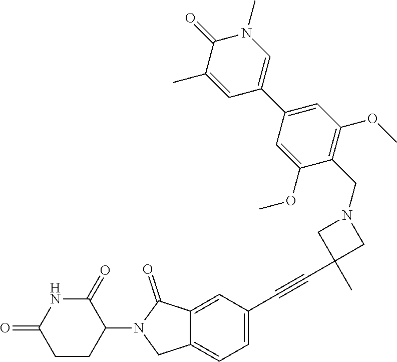 [SMILES: COc1cc(-c2cc(C)c(=O)n(C)c2)cc(OC)c1CN1CC(C)(C#Cc2ccc3c(c2)C(=O)N(C2CCC(=O)NC2=O)C3)C1] |
| D451 | 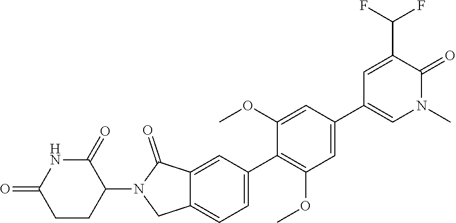 [SMILES: COc1cc(-c2cc(C(F)F)c(=O)n(C)c2)cc(OC)c1-c1ccc2c(c1)C(=O)N(C1CCC(=O)NC1=O)C2] |
| D452 | 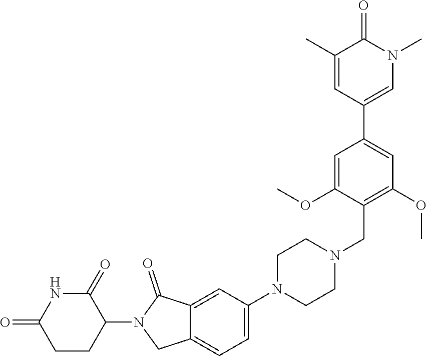 [SMILES: COc1cc(-c2cc(C)c(=O)n(C)c2)cc(OC)c1CN1CCN(c2ccc3c(c2)C(=O)N(C2CCC(=O)NC2=O)C3)CC1] |
| D453 | 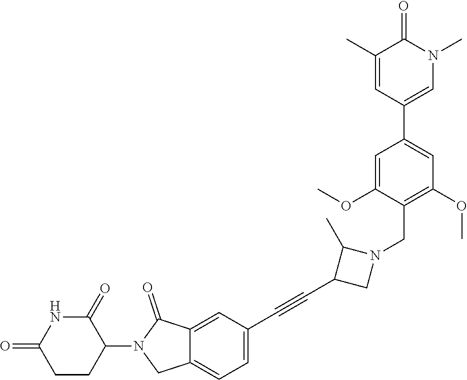 [SMILES: COc1cc(-c2cc(C)c(=O)n(C)c2)cc(OC)c1CN1CC(C#Cc2ccc3c(c2)C(=O)N(C2CCC(=O)NC2=O)C3)C1C] |
| D454 | 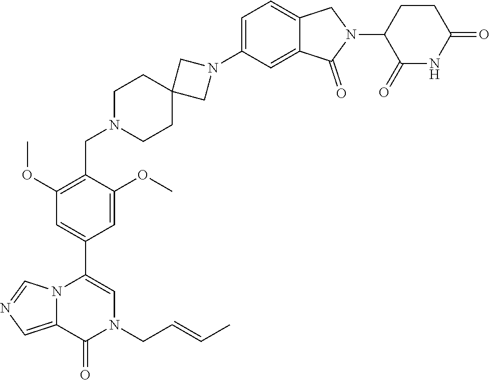 [SMILES: C/C=C/Cn1cc(-c2cc(OC)c(CN3CCC4(CC3)CN(c3ccc5c(c3)C(=O)N(C3CCC(=O)NC3=O)C5)C4)c(OC)c2)n2cncc2c1=O] |
| D455 | 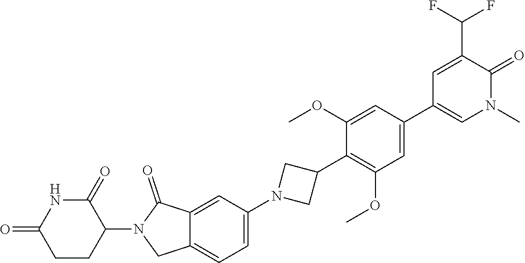 [SMILES: COc1cc(-c2cc(C(F)F)c(=O)n(C)c2)cc(OC)c1C1CN(c2ccc3c(c2)C(=O)N(C2CCC(=O)NC2=O)C3)C1] |
| D456 | 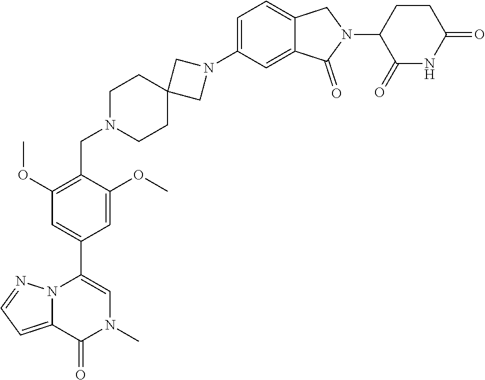 [SMILES: COc1cc(-c2cn(C)c(=O)c3ccnn23)cc(OC)c1CN1CCC2(CC1)CN(c1ccc3c(c1)C(=O)N(C1CCC(=O)NC1=O)C3)C2] |
| D457 | 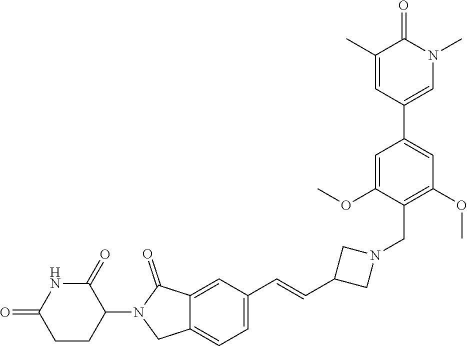 [SMILES: COc1cc(-c2cc(C)c(=O)n(C)c2)cc(OC)c1CN1CC(/C=C/c2ccc3c(c2)C(=O)N(C2CCC(=O)NC2=O)C3)C1] |
| D458 | 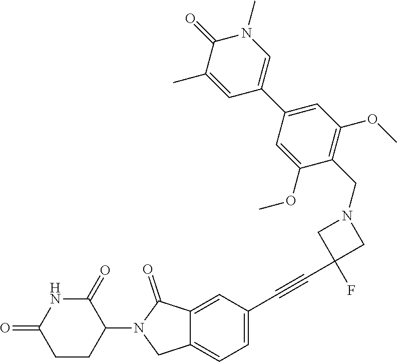 [SMILES: COc1cc(-c2cc(C)c(=O)n(C)c2)cc(OC)c1CN1CC(F)(C#Cc2ccc3c(c2)C(=O)N(C2CCC(=O)NC2=O)C3)C1] |
| D459 | 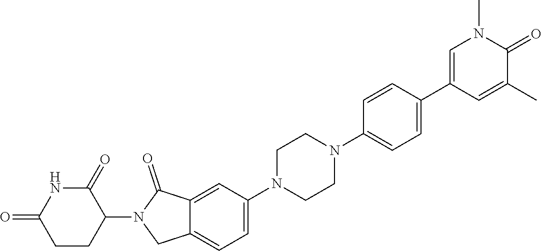 [SMILES: Cc1cc(-c2ccc(N3CCN(c4ccc5c(c4)C(=O)N(C4CCC(=O)NC4=O)C5)CC3)cc2)cn(C)c1=O] |
| D460 | 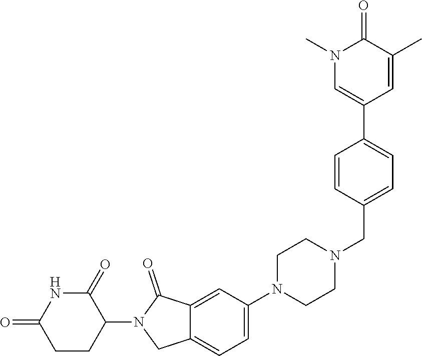 [SMILES: Cc1cc(-c2ccc(CN3CCN(c4ccc5c(c4)C(=O)N(C4CCC(=O)NC4=O)C5)CC3)cc2)cn(C)c1=O] |
| D461 | 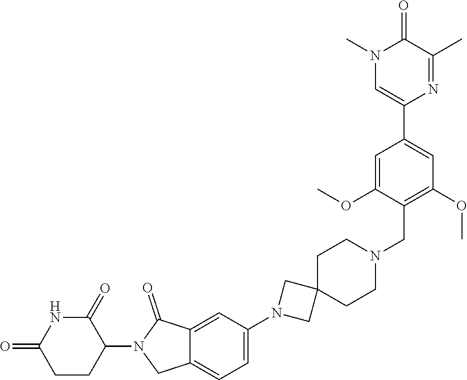 [SMILES: COc1cc(-c2cn(C)c(=O)c(C)n2)cc(OC)c1CN1CCC2(CC1)CN(c1ccc3c(c1)C(=O)N(C1CCC(=O)NC1=O)C3)C2] |
| D462 | 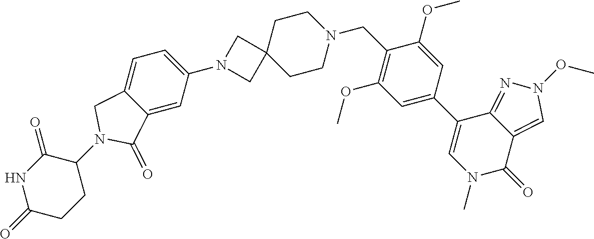 [SMILES: COc1cc(-c2cn(C)c(=O)c3cn(OC)nc23)cc(OC)c1CN1CCC2(CC1)CN(c1ccc3c(c1)C(=O)N(C1CCC(=O)NC1=O)C3)C2] |
| D463 | 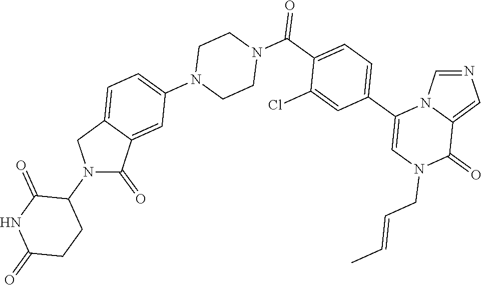 [SMILES: C/C=C/Cn1cc(-c2ccc(C(=O)N3CCN(c4ccc5c(c4)C(=O)N(C4CCC(=O)NC4=O)C5)CC3)c(Cl)c2)n2cncc2c1=O] |
| D464 | 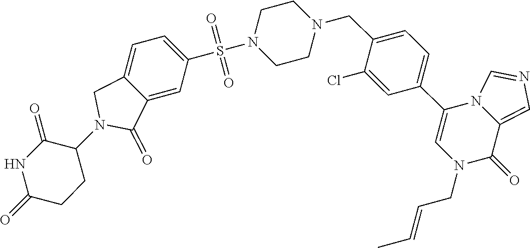 [SMILES: C/C=C/Cn1cc(-c2ccc(CN3CCN(S(=O)(=O)c4ccc5c(c4)C(=O)N(C4CCC(=O)NC4=O)C5)CC3)c(Cl)c2)n2cncc2c1=O] |
| D465 | 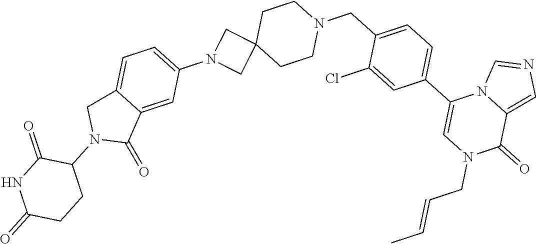 [SMILES: C/C=C/Cn1cc(-c2ccc(CN3CCC4(CC3)CN(c3ccc5c(c3)C(=O)N(C3CCC(=O)NC3=O)C5)C4)c(Cl)c2)n2cncc2c1=O] |
| D466 | 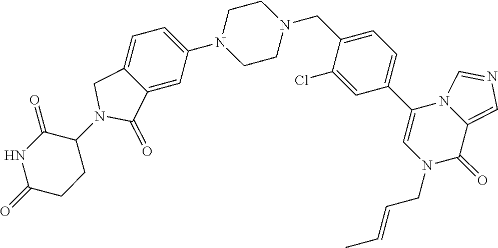 [SMILES: C/C=C/Cn1cc(-c2ccc(CN3CCN(c4ccc5c(c4)C(=O)N(C4CCC(=O)NC4=O)C5)CC3)c(Cl)c2)n2cncc2c1=O] |
| D467 | 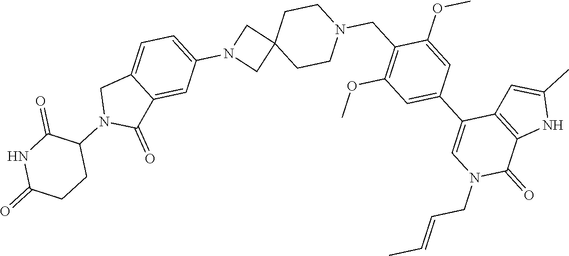 [SMILES: C/C=C/Cn1cc(-c2cc(OC)c(CN3CCC4(CC3)CN(c3ccc5c(c3)C(=O)N(C3CCC(=O)NC3=O)C5)C4)c(OC)c2)c2cc(C)[nH]c2c1=O] |
| D468 | 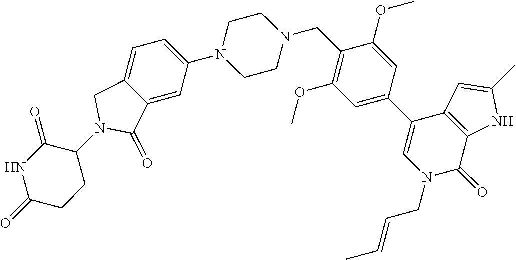 [SMILES: C/C=C/Cn1cc(-c2cc(OC)c(CN3CCN(c4ccc5c(c4)C(=O)N(C4CCC(=O)NC4=O)C5)CC3)c(OC)c2)c2cc(C)[nH]c2c1=O] |
| D469 | 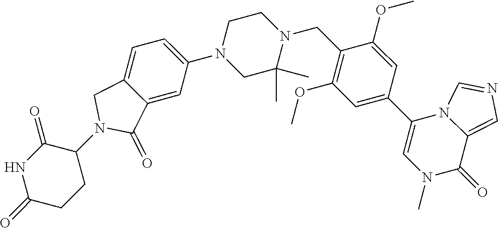 [SMILES: COc1cc(-c2cn(C)c(=O)c3cncn23)cc(OC)c1CN1CCN(c2ccc3c(c2)C(=O)N(C2CCC(=O)NC2=O)C3)CC1(C)C] |
| D470 | 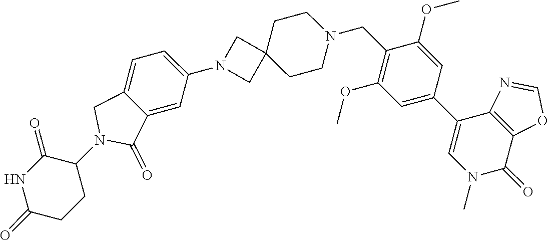 [SMILES: COc1cc(-c2cn(C)c(=O)c3ocnc23)cc(OC)c1CN1CCC2(CC1)CN(c1ccc3c(c1)C(=O)N(C1CCC(=O)NC1=O)C3)C2] |
| D471 | 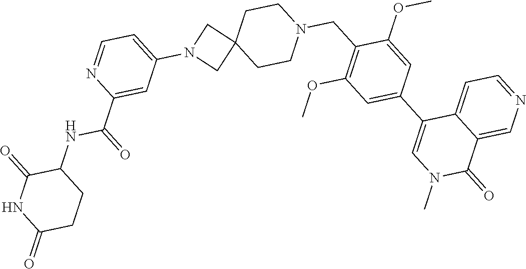 [SMILES: COc1cc(-c2cn(C)c(=O)c3cnccc23)cc(OC)c1CN1CCC2(CC1)CN(c1ccnc(C(=O)NC3CCC(=O)NC3=O)c1)C2] |
| D472 | 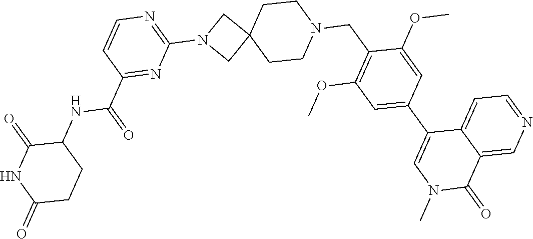 [SMILES: COc1cc(-c2cn(C)c(=O)c3cnccc23)cc(OC)c1CN1CCC2(CC1)CN(c1nccc(C(=O)NC3CCC(=O)NC3=O)n1)C2] |
| D473 | 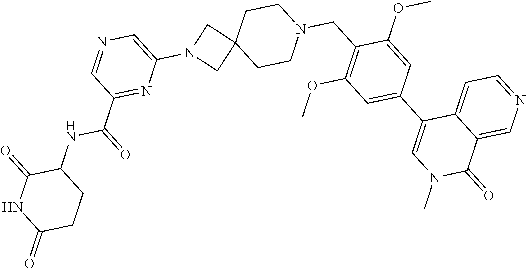 [SMILES: COc1cc(-c2cn(C)c(=O)c3cnccc23)cc(OC)c1CN1CCC2(CC1)CN(c1cncc(C(=O)NC3CCC(=O)NC3=O)n1)C2] |
| D474 | 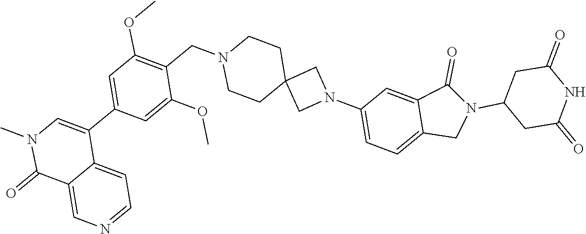 [SMILES: COc1cc(-c2cn(C)c(=O)c3cnccc23)cc(OC)c1CN1CCC2(CC1)CN(c1ccc3c(c1)C(=O)N(C1CC(=O)NC(=O)C1)C3)C2] |

TABLE 1E
Compounds DD11-DD16 of the disclosure

| Compound | |
|---|---|
| No. | Structure |
| DD11 | 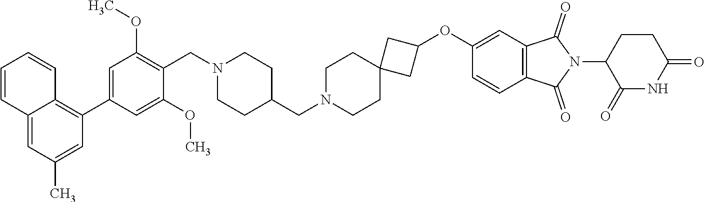 [SMILES: COc1cc(-c2cc(C)cc3ccccc23)cc(OC)c1CN1CCC(CN2CCC3(CC2)CC(Oc2ccc4c(c2)C(=O)N(C2CCC(=O)NC2=O)C4=O)C3)CC1] |
| DD12 | 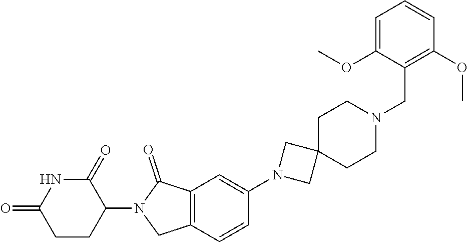 [SMILES: COc1cccc(OC)c1CN1CCC2(CC1)CN(c1ccc3c(c1)C(=O)N(C1CCC(=O)NC1=O)C3)C2] |
| DD13 | 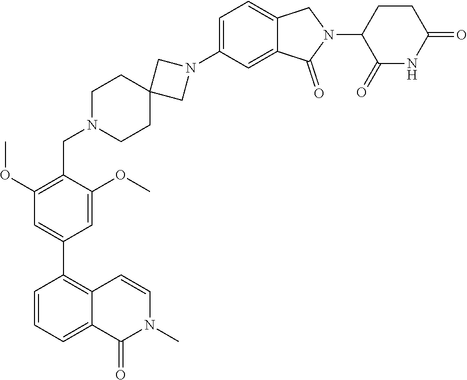 [SMILES: COc1cc(-c2cccc3c(=O)n(C)ccc23)cc(OC)c1CN1CCC2(CC1)CN(c1ccc3c(c1)C(=O)N(C1CCC(=O)NC1=O)C3)C2] |
| DD14 | 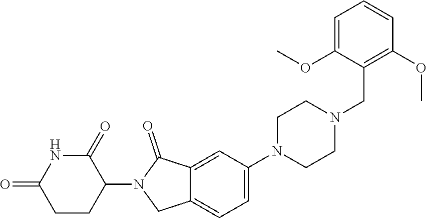 [SMILES: COc1cccc(OC)c1CN1CCN(c2ccc3c(c2)C(=O)N(C2CCC(=O)NC2=O)C3)CC1] |
| DD15 | 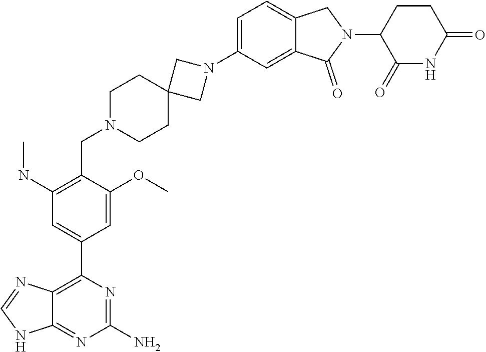 [SMILES: CNc1cc(-c2nc(N)nc3[nH]cnc23)cc(OC)c1CN1CCC2(CC1)CN(c1ccc3c(c1)C(=O)N(C1CCC(=O)NC1=O)C3)C2] |
| DD16 | 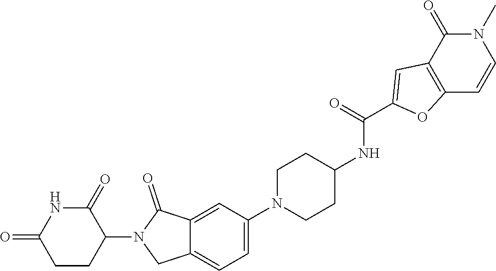 [SMILES: Cn1ccc2oc(C(=O)NC3CCN(c4ccc5c(c4)C(=O)N(C4CCC(=O)NC4=O)C5)CC3)cc2c1=O] |

In another aspect, the disclosure features a pharmaceutical composition including any of the foregoing compounds, or pharmaceutically acceptable salts thereof, and a pharmaceutically acceptable excipient.

In an aspect, the disclosure features a method of inhibiting the level and/or activity of BRD9 in a cell, the method involving contacting the cell with an effective amount of any of the foregoing compounds, or pharmaceutically acceptable salts thereof, or a pharmaceutical composition thereof.

In another aspect, the disclosure features a method of reducing the level and/or activity of BRD9 in a cell, the method involving contacting the cell with an effective amount of any of the foregoing compounds, or pharmaceutically acceptable salts thereof, or a pharmaceutical composition thereof.

In some embodiments, the cell is a cancer cell.

In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, a sarcoma (e.g., a soft tissue sarcoma, synovial sarcoma, Ewing's sarcoma, osteosarcoma, rhabdomyosarcoma, adult fibrosarcoma, alveolar soft-part sarcoma, angiosarcoma, clear cell sarcoma, desmoplastic small round cell tumor, epithelioid sarcoma, fibromyxoid sarcoma, gastrointestinal stromal tumor, Kaposi sarcoma, liposarcoma, leiomyosarcoma, malignant mesenchymoma malignant peripheral nerve sheath tumors, myxofibrosarcoma, low-grade rhabdomyosarcoma), non-small cell lung cancer (e.g., squamous or adenocarcinoma), stomach cancer, or breast cancer. In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, or colorectal cancer. In some embodiments, the cancer is a sarcoma (e.g., synovial sarcoma or Ewing's sarcoma), non-small cell lung cancer (e.g., squamous or adenocarcinoma), stomach cancer, or breast cancer. In some embodiments, the cancer is sarcoma (e.g., synovial sarcoma or Ewing's sarcoma). In some embodiments, the sarcoma is synovial sarcoma.

In an aspect, the disclosure features a method of treating a BAF complex-related disorder in a subject in need thereof, the method involving administering to the subject an effective amount of any of the foregoing compounds, or pharmaceutically acceptable salts thereof, or a pharmaceutical composition thereof. In some embodiments, the BAF complex-related disorder is cancer. In some embodiments, the BAF complex-related disorder is infection.

In another aspect, the disclosure features a method of treating an SS18-SSX fusion protein-related disorder in a subject in need thereof, the method involving administering to the subject an effective amount of any of the foregoing compounds, or pharmaceutically acceptable salts thereof, or a pharmaceutical composition thereof. In some embodiments, the SS18-SSX fusion protein-related disorder is cancer. In some embodiments, the SS18-SSX fusion protein-related disorder is infection. In some embodiments of any of the foregoing methods, the SS18-SSX fusion protein is a SS18-SSX1 fusion protein, a SS18-SSX2 fusion protein, or a SS18-SSX4 fusion protein.

In yet another aspect, the disclosure features a method of treating a BRD9-related disorder in a subject in need thereof, the method involving administering to the subject an effective amount of any of the foregoing compounds, or pharmaceutically acceptable salts thereof, or a pharmaceutical composition thereof. In some embodiments, the BRD9-related disorder is cancer. In some embodiments, the BRD9-related disorder is infection.

In some embodiments, the cancer is squamous cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, a sarcoma (e.g., a soft tissue sarcoma, synovial sarcoma, Ewing's sarcoma, osteosarcoma, rhabdomyosarcoma, adult fibrosarcoma, alveolar soft-part sarcoma, angiosarcoma, clear cell sarcoma, desmoplastic small round cell tumor, epithelioid sarcoma, fibromyxoid sarcoma, gastrointestinal stromal tumor, Kaposi sarcoma, liposarcoma, leiomyosarcoma, malignant mesenchymoma malignant peripheral nerve sheath tumors, myxofibrosarcoma, low-grade rhabdomyosarcoma), non-small cell lung cancer (e.g., squamous or adenocarcinoma), stomach cancer, or breast cancer. In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, or colorectal cancer. In some embodiments, the cancer is a sarcoma (e.g., synovial sarcoma or Ewing's sarcoma), non-small cell lung cancer (e.g., squamous or adenocarcinoma), stomach cancer, or breast cancer. In some embodiments, the cancer is sarcoma (e.g., synovial sarcoma or Ewing's sarcoma). In some embodiments, the sarcoma is synovial sarcoma.

In some embodiments, the infection is viral infection (e.g., an infection with a virus of the Retroviridae family such as the lentiviruses (e.g. Human immunodeficiency virus (HIV) and deltaretroviruses (e.g., human T cell leukemia virus I (HTLV-l), human T cell leukemia virus II (HTLV-II)); Hepadnaviridae family (e.g. hepatitis B virus (HBV)); Flaviviridae family (e.g. hepatitis C virus (HCV)); Adenoviridae family (e.g. Human Adenovirus); Herpesviridae family (e.g. Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus); Papillomaviridae family (e.g. Human Papillomavirus (HPV, HPV E1)); Parvoviridae family (e.g. Parvovirus B19); Polyomaviridae family (e.g. JC virus and BK virus); Paramyxoviridae family (e.g. Measles virus); or Togaviridae family (e.g. Rubella virus)). In some embodiments, the disorder is Coffin Siris, Neurofibromatosis (e.g., NF-1, NF-2, or Schwannomatosis), or Multiple Meningioma.In an aspect, the disclosure features a method of treating a cancer in a subject in need thereof, the method including administering to the subject an effective amount of any of the foregoing compounds, or pharmaceutically acceptable salts thereof, or any of the foregoing pharmaceutical compositions.

In some embodiments, the cancer is squamous cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, a sarcoma (e.g., a soft tissue sarcoma, synovial sarcoma, Ewing's sarcoma, osteosarcoma, rhabdomyosarcoma, adult fibrosarcoma, alveolar soft-part sarcoma, angiosarcoma, clear cell sarcoma, desmoplastic small round cell tumor, epithelioid sarcoma, fibromyxoid sarcoma, gastrointestinal stromal tumor, Kaposi sarcoma, liposarcoma, leiomyosarcoma, malignant mesenchymoma malignant peripheral nerve sheath tumors, myxofibrosarcoma, low-grade rhabdomyosarcoma), non-small cell lung cancer (e.g., squamous or adenocarcinoma), stomach cancer, or breast cancer. In some embodiments, the cancer is a malignant, rhabdoid tumor, a CD8+ T-cell lymphoma, endometrial carcinoma, ovarian carcinoma, bladder cancer, stomach cancer, pancreatic cancer, esophageal cancer, prostate cancer, renal cell carcinoma, melanoma, or colorectal cancer. In some embodiments, the cancer is a sarcoma (e.g., synovial sarcoma or Ewing's sarcoma), non-small cell lung cancer (e.g., squamous or adenocarcinoma), stomach cancer, or breast cancer. In some embodiments, the cancer is sarcoma (e.g., synovial sarcoma or Ewing's sarcoma). In some embodiments, the sarcoma is synovial sarcoma.

In another aspect, the disclosure features a method for treating a viral infection in a subject in need thereof. This method includes administering to the subject an effective amount of any of the foregoing compounds, or pharmaceutically acceptable salts thereof, or any of the foregoing pharmaceutical compositions. In some embodiments, the viral infection is an infection with a virus of the Retroviridae family such as the lentiviruses (e.g. Human immunodeficiency virus (HIV) and deltaretroviruses (e.g., human T cell leukemia virus I (HTLV-l), human T cell leukemia virus II (HTLV-II)); Hepadnaviridae family (e.g. hepatitis B virus (HBV)), Flaviviridae family (e.g. hepatitis C virus (HCV)), Adenoviridae family (e.g. Human Adenovirus), Herpesviridae family (e.g. Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus), Papillomaviridae family (e.g. Human Papillomavirus (HPV, HPV E1)), Parvoviridae family (e.g. Parvovirus B19), Polyomaviridae family (e.g. JC virus and BK virus), Paramyxoviridae family (e.g. Measles virus), Togaviridae family (e.g. Rubella virus).

In another embodiment of any of the foregoing methods, the method further includes administering to the subject an additional anticancer therapy (e.g., chemotherapeutic or cytotoxic agent or radiotherapy).

In particular embodiments, the additional anticancer therapy is: a chemotherapeutic or cytotoxic agent (e.g., doxorubicin or ifosfamide), a differentiation-inducing agent (e.g., retinoic acid, vitamin D, cytokines), a hormonal agent, an immunological agent, or an anti-angiogenic agent. Chemotherapeutic and cytotoxic agents include, but are not limited to, alkylating agents, cytotoxic antibiotics, antimetabolites, vinca alkaloids, etoposides, and others (e.g., paclitaxel, taxol, docetaxel, taxotere, cis-platinum). A list of additional compounds having anticancer activity can be found in L. Brunton, B. Chabner and B. Knollman (eds). Goodman and Gilman's The Pharmacological Basis of Therapeutics, Twelfth Edition, 2011, McGraw Hill Companies, New York, NY.

In particular embodiments, the compound of the invention and the additional anticancer therapy and any of the foregoing compounds or pharmaceutical compositions are administered within 28 days of each other (e.g., within 21, 14, 10, 7, 5, 4, 3, 2, or 1 days) or within 24 hours (e.g., 12, 6, 3, 2, or 1 hours; or concomitantly) each in an amount that together are effective to treat the subject.

### Chemical Terms

The terminology employed herein is for the purpose of describing particular embodiments and is not intended to be limiting.

For any of the following chemical definitions, a number following an atomic symbol indicates that total number of atoms of that element that are present in a particular chemical moiety. As will be understood, other atoms, such as hydrogen atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The term “aliphatic,” as used herein, refers to a saturated or unsaturated, straight, branched, or cyclic hydrocarbon. “Aliphatic” is intended herein to include, but is not limited to, alkyl, alkenyl, alkynyl, cycloalkyl, cycloalkenyl, and cycloalkynyl moieties, and thus incorporates each of these definitions. In one embodiment, “aliphatic” is used to indicate those aliphatic groups having 1-20 carbon atoms. The aliphatic chain can be, for example, mono-unsaturated, di-unsaturated, tri-unsaturated, or polyunsaturated, or alkynyl. Unsaturated aliphatic groups can be in a cis or trans configuration. In one embodiment, the aliphatic group contains from 1 to about 12 carbon atoms, more generally from 1 to about 6 carbon atoms or from 1 to about 4 carbon atoms. In one embodiment, the aliphatic group contains from 1 to about 8 carbon atoms. In certain embodiments, the aliphatic group is C₁-C₂, C₁-C₃, C₁-C₄, C₁-C₅, or C₁-C₆. The specified ranges as used herein indicate an aliphatic group having each member of the range described as an independent species. For example, the term C₁-C₆ aliphatic as used herein indicates a straight or branched alkyl, alkenyl, or alkynyl group having from 1, 2, 3, 4, 5, or 6 carbon atoms and is intended to mean that each of these is described as an independent species. For example, the term C₁-C₄ aliphatic as used herein indicates a straight or branched alkyl, alkenyl, or alkynyl group having from 1, 2, 3, or 4 carbon atoms and is intended to mean that each of these is described as an independent species. In one embodiment, the aliphatic group is substituted with one or more functional groups that results in the formation of a stable moiety.

The term “heteroaliphatic,” as used herein, refers to an aliphatic moiety that contains at least one heteroatom in the chain, for example, an amine, carbonyl, carboxy, oxo, thio, phosphate, phosphonate, nitrogen, phosphorus, silicon, or boron atoms in place of a carbon atom. In one embodiment, the only heteroatom is nitrogen. In one embodiment, the only heteroatom is oxygen. In one embodiment, the only heteroatom is sulfur. “Heteroaliphatic” is intended herein to include, but is not limited to, heteroalkyl, heteroalkenyl, heteroalkynyl, heterocycloalkyl, heterocycloalkenyl, and heterocycloalkynyl moieties. In one embodiment, “heteroaliphatic” is used to indicate a heteroaliphatic group (cyclic, acyclic, substituted, unsubstituted, branched or unbranched) having 1-20 carbon atoms. In one embodiment, the heteroaliphatic group is optionally substituted in a manner that results in the formation of a stable moiety. Nonlimiting examples of heteroaliphatic moieties are polyethylene glycol, polyalkylene glycol, amide, polyamide, polylactide, polyglycolide, thioether, ether, alkyl-heterocycle-alkyl, —O-alkyl-O-alkyl, and alkyl-O-haloalkyl.

The term “acyl,” as used herein, represents a hydrogen or an alkyl group that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms). An “alkylene” is a divalent alkyl group.

The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms). An “alkenylene” is a divalent alkenyl group.

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms). An “alkynylene” is a divalent alkynyl group.

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the compounds described herein can be an unsubstituted amino (i.e., —NH₂) or a substituted amino (i.e., —N(R^{N1})₂).

The term “aryl,” as used herein, refers to an aromatic mono- or polycarbocyclic radical of, e.g., 6 to 12, carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged cyclyl,” as used herein, refers to a bridged polycyclic group of 5 to 20 atoms, containing from 1 to 3 bridges. Bridged cyclyl includes bridged carbocyclyl (e.g., norbornyl) and bridged heterocyclyl (e.g., 1,4-diazabicyclo[2.2.2]octane).

The term “cyano,” as used herein, represents a —CN group.

The term “carbocyclyl,” as used herein, refers to a non-aromatic C₃-C₁₂, monocyclic or polycyclic (e.g., bicyclic or tricyclic) structure in which the rings are formed by carbon atoms. Carbocyclyl structures include cycloalkyl groups (e.g., cyclohexyl) and unsaturated carbocyclyl radicals (e.g., cyclohexenyl). Polycyclic carbocyclyl includes spirocyclic carbocyclyl, bridged carbocyclyl, and fused carbocyclyl. A “carbocyclylene” is a divalent carbocyclyl group.

The term “cycloalkyl,” as used herein, refers to a saturated, non-aromatic, monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

The terms “halo” or “halogen,” as used herein, mean a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers to alkyl-O— (e.g., methoxy and ethoxy), and an “alkylamino” which, as used herein, refers to —N(alkyl)R^{Na}, where R^{Na} is H or alkyl (e.g., methylamino). A “heteroalkylene” is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers to alkenyl-O—. A “heteroalkenylene” is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers to alkynyl-O—. A “heteroalkynylene” is a divalent heteroalkynyl group.

The term “heteroaryl,” as used herein, refers to an aromatic monocyclic or polycyclic structure of to 12 atoms having at least one aromatic ring containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl. A “heteroarylene” is a divalent heteroaryl group.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, refers a monocyclic or polycyclic radical (e.g., bicyclic or tricyclic) having 3 to 12 atoms having at least one non-aromatic ring containing 1, 2, 3, or 4 ring atoms selected from N, O, or S, and no aromatic ring containing any N, O, or S atoms. Polycyclic heterocyclyl includes spirocyclic heterocyclyl, bridged heterocyclyl, and fused heterocyclyl. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl. A “heterocyclylene” is a divalent heterocyclyl group.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an —OH group.

The term “hydroxyl,” as used herein, represents an —OH group.

The term “imine,” as used herein, represents ═NR^{N} group, where R^{N} is, e.g., H or alkyl.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, a-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, a,a-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “oxo,” as used herein, represents an ═O group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, oxo, sulfonyl, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds described herein (e.g., compounds of the invention) can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds described herein (e.g., the compounds of the invention) may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²p, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy.

Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “including” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “adult soft tissue sarcoma” refers to a sarcoma that develops in the soft tissues of the body, typically in adolescent and adult subjects (e.g., subjects who are at least 10 years old, 11 years old, 12 years old, 13 years old, 14 years old, 15 years old, 16 years old, 17 years old, 18 years old, or 19 years old). Non-limiting examples of adult soft tissue sarcoma include, but are not limited to, synovial sarcoma, fibrosarcoma, malignant fibrous histiocytoma, dermatofibrosarcoma, liposarcoma, leiomyosarcoma, hemangiosarcoma, Kaposi's sarcoma, lymphangiosarcoma, malignant peripheral nerve sheath tumor/neurofibrosarcoma, extraskeletal chondrosarcoma, extraskeletal osteosarcoma, extraskeletal myxoid chondrosarcoma, and extraskeletal mesenchymal.

The term “antisense,” as used herein, refers to a nucleic acid comprising a polynucleotide that is sufficiently complementary to all or a portion of a gene, primary transcript, or processed mRNA, so as to interfere with expression of the endogenous gene (e.g., BRD9). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other.

The term “antisense nucleic acid” includes single-stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity (e.g., 80%, 85%, 86%, 87%, 88%, 89%, 90%, 91%, 92%, 93%, 94%, 95%, 96%, 97%, 98%, 99%, 99.9% identity, or more) with the targeted polypeptide sequence (e.g., a BRD9 polypeptide sequence). The antisense nucleic acid can be complementary to an entire coding strand, or to only a portion thereof. In some embodiments, an antisense nucleic acid molecule is antisense to a “coding region” of the coding strand of a nucleotide sequence. The term “coding region” refers to the region of the nucleotide sequence comprising codons that are translated into amino acid residues. In some embodiments, the antisense nucleic acid molecule is antisense to a “noncoding region” of the coding strand of a nucleotide sequence. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of mRNA, or can be antisense to only a portion of the coding or noncoding region of an mRNA. For example, the antisense oligonucleotide can be complementary to the region surrounding the translation start site. An antisense oligonucleotide can be, for example, about 5, 10, 15, 20, 25, 30, 35, 40, 45, or 50 nucleotides in length.

As used herein, the term “BAF complex” refers to the BRG1- or HRBM-associated factors complex in a human cell.

As used herein, the term “BAF complex-related disorder” refers to a disorder that is caused or affected by the level and/or activity of a BAF complex.

As used herein, the terms “GBAF complex” and “GBAF” refer to a SWI/SNF ATPase chromatin remodeling complex in a human cell. GBAF complex subunits may include, but are not limited to, ACTB, ACTL6A, ACTL6B, BICRA, BICRAL, BRD9, SMARCA2, SMARCA4, SMARCC1, SMARCD1, SMARCD2, SMARCD3, and SS18. The term “cancer” refers to a condition caused by the proliferation of malignant neoplastic cells, such as tumors, neoplasms, carcinomas, sarcomas, leukemias, and lymphomas.

As used herein, the term “BRD9” refers to bromodomain-containing protein 9, a component of the BAF (BRG1- or BRM-associated factors) complex, a SWI/SNF ATPase chromatin remodeling complex, and belongs to family IV of the bromodomain-containing proteins. BRD9 is encoded by the BRD9 gene, the nucleic acid sequence of which is set forth in SEQ ID NO: 1. The term “BRD9” also refers to natural variants of the wild-type BRD9 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type BRD9.

As used herein, the term “BRD9-related disorder” refers to a disorder that is caused or affected by the level and/or activity of BRD9. The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

A “compound of the present invention” and similar terms as used herein, whether explicitly noted or not, refers to compounds useful for treating BAF-related disorders (e.g., cancer or infection) described herein, including, e.g., compounds of Formula I (e.g., compounds of Table 1A, Table 1B, and Table 1D) and compounds of Table 1C and 1E, as well as salts (e.g., pharmaceutically acceptable salts), solvates, hydrates, stereoisomers (including atropisomers), and tautomers thereof. Those skilled in the art will appreciate that certain compounds described herein can exist in one or more different isomeric (e.g., stereoisomers, geometric isomers, atropisomers, and tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination. Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms. In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

As used herein, the term “degrader” refers to a small molecule compound including a degradation moiety, wherein the compound interacts with a protein (e.g., BRD9) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BRD9. In one example, the moiety binds to a protease or a ubiquitin ligase that metabolizes the protein, e.g., BRD9.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

As used herein, the terms “effective amount,” “therapeutically effective amount,” and “a “sufficient amount” of an agent that reduces the level and/or activity of BRD9 (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating cancer, it is an amount of the agent that reduces the level and/or activity of BRD9 sufficient to achieve a treatment response as compared to the response obtained without administration of the agent that reduces the level and/or activity of BRD9. The amount of a given agent that reduces the level and/or activity of BRD9 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of BRD9 of the present disclosure is an amount which results in a beneficial or desired result in a subject as compared to a control. As defined herein, a therapeutically effective amount of an agent that reduces the level and/or activity of BRD9 of the present disclosure may be readily determined by one of ordinary skill by routine methods known in the art. Dosage regimen may be adjusted to provide the optimum therapeutic response.

As used herein, the term “inhibitor” refers to any agent which reduces the level and/or activity of a protein (e.g., BRD9). Non-limiting examples of inhibitors include small molecule inhibitors, degraders, antibodies, enzymes, or polynucleotides (e.g., siRNA).

The term “inhibitory RNA agent” refers to an RNA, or analog thereof, having sufficient sequence complementarity to a target RNA to direct RNA interference. Examples also include a DNA that can be used to make the RNA. RNA interference (RNAi) refers to a sequence-specific or selective process by which a target molecule (e.g., a target gene, protein, or RNA) is down-regulated. Generally, an interfering RNA (“iRNA”) is a double-stranded short-interfering RNA (siRNA), short hairpin RNA (shRNA), or single-stranded micro-RNA (miRNA) that results in catalytic degradation of specific mRNAs, and also can be used to lower or inhibit gene expression.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, pg/mL, ng/mL) or percentage relative to total protein or mRNA in a sample.

The terms “miRNA” and “microRNA” refer to an RNA agent, preferably a single-stranded agent, of about 10-50 nucleotides in length, preferably between about 15-25 nucleotides in length, which is capable of directing or mediating RNA interference. Naturally-occurring miRNAs are generated from stem-loop precursor RNAs (i.e., pre-miRNAs) by Dicer. The term “Dicer” as used herein, includes Dicer as well as any Dicer ortholog or homolog capable of processing dsRNA structures into siRNAs, miRNAs, siRNA-like or miRNA-like molecules. The term microRNA (“miRNA”) is used interchangeably with the term “small temporal RNA” (“stRNA”) based on the fact that naturally-occurring miRNAs have been found to be expressed in a temporal fashion (e.g., during development).

By “modulating the activity of a BAF complex,” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference. “Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows: 100 multiplied by (the fraction X/Y)

$$10 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

By “reducing the activity of BRD9,” is meant decreasing the level of an activity related to an BRD9, or a related downstream effect. A non-limiting example of inhibition of an activity of BRD9 is decreasing the level of a BAF complex (e.g., GBAF) in a cell. The activity level of BRD9 may be measured using any method known in the art. In some embodiments, an agent which reduces the activity of BRD9 is a small molecule BRD9 inhibitor. In some embodiments, an agent which reduces the activity of BRD9 is a small molecule BRD9 degrader.

By “reducing the level of BRD9,” is meant decreasing the level of BRD9 in a cell or subject. The level of BRD9 may be measured using any method known in the art.

By a “reference” is meant any useful reference used to compare protein or mRNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

The terms “short interfering RNA” and “siRNA” (also known as “small interfering RNAs”) refer to an RNA agent, preferably a double-stranded agent, of about 10-50 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., Dicer or a homolog thereof).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the term “SS18-SSX fusion protein-related disorder” refers to a disorder that is caused or affected by the level and/or activity of SS18-SSX fusion protein.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results.

Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

The details of one or more embodiments of the invention are set forth in the description below. Other features, objects, and advantages of the invention will be apparent from the description and from the claims.

### BRIEF DESCRIPTION OF THE DRAWINGS

FIG. 1 is a series of graphs illustrating the effect of specific guide RNA (sgRNA) targeting of the BRD9 BAF complex subunit on synovial sarcoma cell growth. The Y-axis indicated the dropout ratio. The X-axis indicates the nucleotide position of the BRD9 gene. The grey box indicates the range of the negative control sgRNAs in the screen. The SYO1 cell line carries SS18-SSX2 fusion protein. The breakpoint joining the N-terminal region of SS18 to the C-terminal region of SSX2 are indicated by the black lines in their respective panel. The linear protein sequence is show with BRD9 PFAM domains annotated from the PFAM database.

FIG. 2 is an image illustrating dose dependent depletion of BRD9 levels in a synovial sarcoma cell line (SYO1) in the presence of a BRD9 degrader.

FIG. 3 is an image illustrating sustained suppression of BRD9 levels in a synovial sarcoma cell line (SYO1) in the presence of a BRD9 degrader over 72 hours.

FIG. 4 is an image illustrating sustained suppression of BRD9 levels in two cell lines (293T and SYO1) in the presence of a BRD9 degrader over 5 days.

FIG. 5 is an image illustrating sustained suppression of BRD9 levels in synovial sarcoma cell lines (SYO1 and Yamato) in the presence of a BRD9 degrader over 7 days compared to the levels in cells treated with CRISPR reagents.

FIG. 6 is an image illustrating the effect on cell growth of six cell lines (SYO1, Yamato, A549, HS-SY-II, ASKA, and 293T) in the presence of a BRD9 degrader and a BRD9 inhibitor.

FIG. 7 is an image illustrating the effect on cell growth of two cell lines (SYO1 and G401) in the presence of a BRD9 degrader.

FIG. 8 is an image illustrating the effect on cell growth of three synovial sarcoma cell lines (SYO1, HS-SY-II, and ASKA) in the presence of a BRD9 degrader, BRD9 binder and E3 ligase binder.

FIG. 9 is an image illustrating the effect on cell growth of three non-synovial sarcoma cell lines (RD, HCT116, and Calu6) in the presence of a BRD9 degrader, BRD9 binder and E3 ligase binder.

FIG. 10 is a graph illustrating the percentage of SYO1 in various cell cycle phases following treatment with DMSO, Compound 1 at 200 nM, or Compound 1 at 1 μM for 8 or 13 days.

FIG. 11 is a series of contour plots illustrating the percentage of SYO1 cells in various cell cycle phases following treatment with DMSO, Compound 1 at 200 nM, Compound 1 at 1 μM, or lenalidomide at 200 nM for 8 days. Numerical values corresponding to each contour plot are found in the table below.

FIG. 12 is a series of contour plots illustrating the percentage of SYO1 cells in various cell cycle phases following treatment with DMSO, Compound 1 at 200 nM, Compound 1 at 1 μM, or lenalidomide at 200 nM for 13 days. Numerical values corresponding to each contour plot are found in the table below.

FIG. 13 is a series of contour plots illustrating the percentage of early- and late-apoptotic SYO1 cells following treatment with DMSO, Compound 1 at 200 nM, Compound 1 at 1 μM, or lenalidomide at 200 nM for 8 days. Numerical values corresponding to each contour plot are found in the table below.

FIG. 14 is a graph illustrating the proteins present in BAF complexes including the SS18-SSX fusion protein.

### DETAILED DESCRIPTION

The present disclosure features compositions and methods useful for the treatment of BAF-related disorders (e.g., cancer and infection). The disclosure further features compositions and methods useful for inhibition of the level and/or activity of BRD9, e.g., for the treatment of disorders such as cancer (e.g., sarcoma) and infection (e.g., viral infection), e.g., in a subject in need thereof.

### Compounds

Compounds described herein reduce the level of an activity related to BRD9, or a related downstream effect, or reduce the level of BRD9 in a cell or subject. Exemplary compounds described herein have the structure according to Formula I.

Formula I is

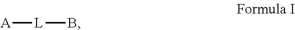

where
A is a BRD9 binding moiety;
B is a degradation moiety; and
L has the structure of Formula II:

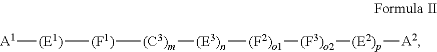

wherein
A¹ is a bond between the linker and A;
A² is a bond between B and the linker;
each of m, n, o1, o2, and p is, independently, 0 or 1;
each of E¹ and E² is, independently, O, S, NR^{N}, optionally substituted C₁₋₁₀ alkyl, optionally substituted C₂₋₁₀ alkenyl, optionally substituted C₂₋₁₀ alkynyl, optionally substituted C₂-C₁₀ polyethylene glycol, or optionally substituted C₁₋₁₀ heteroalkyl;
E³ is O, S, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;
C³ is carbonyl, thiocarbonyl, sulphonyl, or phosphoryl; and
each of F¹, F², and F³ is, independently, optionally substituted C₃-C₁₀ carbocyclyl, optionally substituted C₂-C₉ heterocyclyl, optionally substituted C₆-C₁₀ aryl, or optionally substituted C₂-C₉ heteroaryl, or a pharmaceutically acceptable salt thereof.

### Pharmaceutical Uses

The compounds described herein are useful in the methods of the invention and, while not bound by theory, are believed to exert their desirable effects through their ability to modulate the level, status, and/or activity of a BAF complex, e.g., by inhibiting the activity or level of the BRD9 protein in a cell within the BAF complex in a mammal.

An aspect of the present invention relates to methods of treating disorders related to BRD9 such as cancer in a subject in need thereof. In some embodiments, the compound is administered in an amount and for a time effective to result in one of (or more, e.g., two or more, three or more, four or more of): (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, and (i) increased progression free survival of a subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound described herein. An increase in average survival time of a population may also be measured, for example, by calculating for a population the average length of survival following completion of a first round of treatment with a pharmaceutically acceptable salt of a compound described herein.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of a compound described herein. A decrease in the mortality rate of a population may also be measured, for example, by calculating for a population the average number of disease-related deaths per unit time following completion of a first round of treatment with a pharmaceutically acceptable salt of a compound described herein.

### Combination Therapies

A method of the invention can be used alone or in combination with an additional therapeutic agent, e.g., other agents that treat cancer or symptoms associated therewith, or in combination with other types of therapies to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6 (2005)). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al., Proc. Am. Soc. Clin. Oncol. 18:233a (1999), and Douillard et al., Lancet 355(9209):1041-1047 (2000).

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab-1-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOLIRIS® (eculizumab); CIMZIA® (certolizumab pegol); SIMPONI® (golimumab); ILARIS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

The second agent may be a therapeutic agent which is a non-drug treatment. For example, the second therapeutic agent is radiation therapy, cryotherapy, hyperthermia, and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody or fusion a protein such as ipilimumab/YERVOY® or tremelimumab). In some embodiments, the inhibitor of checkpoint is an inhibitor (e.g., an inhibitory antibody or small molecule inhibitor) of PD-1 (e.g., nivolumab/OPDIVO®; pembrolizumab/KEYTRUDA®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In some embodiments, the anti-cancer therapy is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The pharmaceutical compositions described herein are preferably formulated into pharmaceutical compositions for administration to human subjects in a biologically compatible form suitable for administration in vivo.

The compounds described herein may be used in the form of the free base, in the form of salts, solvates, and as prodrugs. All forms are within the methods described herein. In accordance with the methods of the invention, the described compounds or salts, solvates, or prodrugs thereof may be administered to a patient in a variety of forms depending on the selected route of administration, as will be understood by those skilled in the art. The compounds described herein may be administered, for example, by oral, parenteral, buccal, sublingual, nasal, rectal, patch, pump, intratumoral, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound described herein may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound described herein may be incorporated with an excipient and used in the form of ingestible tablets, buccal tablets, troches, capsules, elixirs, suspensions, syrups, and wafers. A compound described herein may also be administered parenterally. Solutions of a compound described herein can be prepared in water suitably mixed with a surfactant, such as hydroxypropylcellulose.

Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. A compound described herein may be administered intratumorally, for example, as an intratumoral injection.

Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature.

The compounds described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds described herein, and/or compositions including a compound described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds described herein may be administered initially in a suitable dosage that may be adjusted as required, depending on the clinical response. In general, satisfactory results may be obtained when the compounds described herein are administered to a human at a daily dosage of, for example, between 0.05 mg and 3000 mg (measured as the solid form). Dose ranges include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

Alternatively, the dosage amount can be calculated using the body weight of the patient. For example, the dose of a compound, or pharmaceutical composition thereof, administered to a patient may range from 0.1-100 mg/kg (e.g., 0.1-50 mg/kg (e.g., 0.25-25 mg/kg)). In exemplary, non-limiting embodiments, the dose may range from 0.5-5.0 mg/kg (e.g., 0.5, 1.0, 1.5, 2.0, 2.5, 3.0, 3.5, 4.0, 4.5, or 5.0 mg/kg) or from 5.0-20 mg/kg (e.g., 5.5, 6.0, 6.5, 7.0, 7.5, 8.0, 8.5, 9.0, 9.5, 10, 11, 12, 13, 14, 15, 16, 17, 18, 19, or 20 mg/kg).

### Kits

The invention also features kits including (a) a pharmaceutical composition including an agent that reduces the level and/or activity of BRD9 in a cell or subject described herein, and (b) a package insert with instructions to perform any of the methods described herein. In some embodiments, the kit includes (a) a pharmaceutical composition including an agent that reduces the level and/or activity of BRD9 in a cell or subject described herein, (b) an additional therapeutic agent (e.g., an anti-cancer agent), and (c) a package insert with instructions to perform any of the methods described herein.

### EXAMPLES

### Example 1—High Density Tiling sgRNA Screen Against Human BAF Complex Subunits in Synovial Sarcoma Cell Line SYO1

The following example shows that BRD9 sgRNA inhibits cell growth in synovial sarcoma cells.

### Procedure:

To perform high density sgRNA tiling screen, an sgRNA library against BAF complex subunits was custom synthesized at Cellecta (Mountain View, CA). Sequences of DNA encoding the BRD9-targeting sgRNAs used in this screen are listed in Table 2. Negative and positive control sgRNA were included in the library. Negative controls consisted of 200 sgRNAs that do not target human genome. The positive controls are sgRNAs targeting essential genes (CDC16, GTF2B, HSPA5, HSPA9, PAFAH1 B1, PCNA, POLR2L, RPL9, and SF3A3). DNA sequences encoding all positive and negative control sgRNAs are listed in Table 3. Procedures for virus production, cell infection, and performing the sgRNA screen were previously described (Tsherniak et al, Cell 170:564-576 (2017); Munoz et al, Cancer Discovery 6:900-913 (2016)). For each sgRNA, 50 counts were added to the sequencing counts and for each time point the resulting counts were normalized to the total number of counts. The log 2 of the ratio between the counts (defined as dropout ratio) at day 24 and day 1 post-infection was calculated. For negative control sgRNAs, the 2.5 and 97.5 percentile of the log 2 dropout ratio of all non-targeting sgRNAs was calculated and considered as background (grey box in the graph). Protein domains were obtained from PFAM regions defined for the UNIPROT identifier: Q9H8M2.

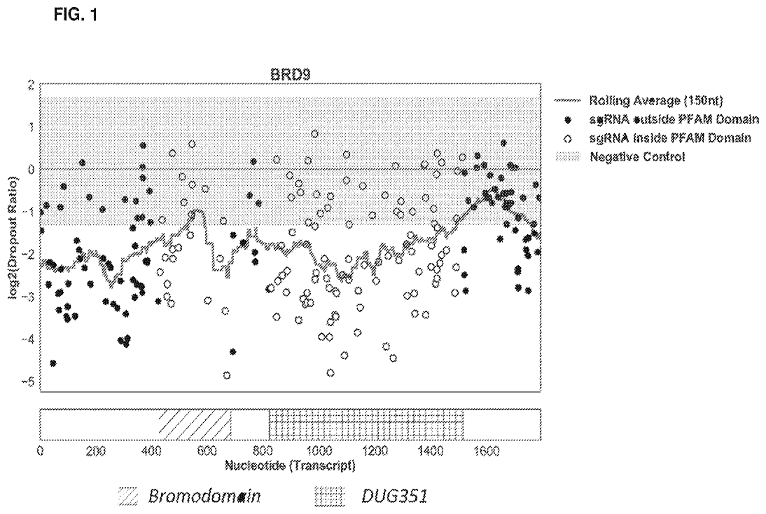

Results: As shown in FIG. 1, targeted inhibition of the GBAF complex component BRD9 by sgRNA resulted in growth inhibition of the SYO1 synovial sarcoma cell line. sgRNAs against other components of the BAF complexes resulted in increased proliferation of cells, inhibition of cell growth, or had no effect on SYO1 cells. These data show that targeting various subunits of the GBAF complex represents a therapeutic strategy for the treatment of synovial sarcoma.

TABLE 2
BRD9 sgRNA Library

| SEQ ID NO | Nucleic Acid Sequence |
|---|---|
| 203 | CAAGAAGCACAAGAAGCACA |
| 204 | CTTGTGCTTCTTGCCCATGG |
| 205 | CTTCTTGTGCTTCTTGCCCA |
| 206 | ACAAGAAGCACAAGGCCGAG |
| 207 | CTCGTAGGACGAGCGCCACT |
| 208 | CGAGTGGCGCTCGTCCTACG |
| 209 | GAGTGGCGCTCGTCCTACGA |
| 210 | AGGCTTCTCCAGGGGCTTGT |
| 211 | AGATTATGCCGACAAGCCCC |
| 212 | ACCTTCAGGACTAGCTTTAG |
| 213 | AGCTTTAGAGGCTTCTCCAG |
| 214 | CTAGCTTTAGAGGCTTCTCC |
| 215 | TAGCTTTAGAGGCTTCTCCA |
| 216 | CTAAAGCTAGTCCTGAAGGT |
| 217 | GCCTCTAAAGCTAGTCCTGA |
| 218 | CTTCACTTCCTCCGACCTTC |
| 219 | AAGCTAGTCCTGAAGGTCGG |
| 220 | AGTGAAGTGACTGAACTCTC |
| 221 | GTGACTGAACTCTCAGGATC |
| 222 | ATAGTAACTGGAGTCGTGGC |
| 223 | CATCATAGTAACTGGAGTCG |
| 224 | TGACCTGTCATCATAGTAAC |
| 225 | ACTCCAGTTACTATGATGAC |
| 226 | CTTTGTGCCTCTCTCGCTCA |
| 227 | GGTCAGACCATGAGCGAGAG |
| 228 | GAAGAAGAAGAAGTCCGAGA |
| 229 | GTCCAGATGCTTCTCCTTCT |
| 230 | GTCCGAGAAGGAGAAGCATC |
| 231 | GGAGAAGCATCTGGACGATG |
| 232 | TGAGGAAAGAAGGAAGCGAA |
| 233 | ATCTGGACGATGAGGAAAGA |
| 234 | AGAAGAAGCGGAAGCGAGAG |
| 235 | GAAGAAGCGGAAGCGAGAGA |
| 236 | CCGCCCAGGAAGAGAAGAAG |
| 237 | AGAGAGGGAGCACTGTGACA |
| 238 | AGGGAGCACTGTGACACGGA |
| 239 | GAGGGAGCACTGTGACACGG |
| 240 | GCACTGTGACACGGAGGGAG |
| 241 | GAGGCTGACGACTTTGATCC |
| 242 | AGGCTGACGACTTTGATCCT |
| 243 | TCCACCTCCACCTTCTTCCC |
| 244 | CGACTTTGATCCTGGGAAGA |
| 245 | CTTTGATCCTGGGAAGAAGG |
| 246 | TGATCCTGGGAAGAAGGTGG |
| 247 | TCCTGGGAAGAAGGTGGAGG |
| 248 | CGGACTGGCCGATCTGGGGG |
| 249 | ACGCTCGGACTGGCCGATCT |
| 250 | AGGTGGAGCCGCCCCCAGAT |
| 251 | CGCTCGGACTGGCCGATCTG |
| 252 | GCTCGGACTGGCCGATCTGG |
| 253 | CACGCTCGGACTGGCCGATC |
| 254 | TGTGTCCGGCACGCTCGGAC |
| 255 | CTGGCTGTGTCCGGCACGCT |
| 256 | ATCGGCCAGTCCGAGCGTGC |
| 257 | CACCCTTGCCTGGCTGTGTC |
| 258 | CGAGCGTGCCGGACACAGCC |
| 259 | TGTTCCAGGAGTTGCTGAAT |
| 260 | CACACCTATTCAGCAACTCC |
| 261 | GCTGGCGGAGGAAGTGTTCC |
| 262 | TTTACCTCTGAAGCTGGCGG |
| 263 | CCCCGGTTTACCTCTGAAGC |
| 264 | ACTTCCTCCGCCAGCTTCAG |
| 265 | CAGGAAAAGCAAAAAATCCA |
| 266 | GCTTTCAGAAAAGATCCCCA |
| 267 | AGGAAAAGCAAAAAATCCAT |
| 268 | GGAAAAGCAAAAAATCCATG |
| 269 | GGAGCAATTGCATCCGTGAC |
| 270 | GTCACGGATGCAATTGCTCC |
| 271 | TTTATTATCATTGAATATCC |
| 272 | AATGATAATAAAACATCCCA |
| 273 | ATAAAACATCCCATGGATTT |
| 274 | TTCATGGTGCCAAAATCCAT |
| 275 | TTTCATGGTGCCAAAATCCA |
| 276 | TAATGAATACAAGTCAGTTA |
| 277 | CAAGTCAGTTACGGAATTTA |
| 278 | ATAATGCAATGACATACAAT |
| 279 | AACTTGTAGTACACGGTATC |
| 280 | CTTCGCCAACTTGTAGTACA |
| 281 | AGATACCGTGTACTACAAGT |
| 282 | GCGAAGAAGATCCTTCACGC |
| 283 | TCATCTTAAAGCCTGCGTGA |
| 284 | TTCTCAGCAGGCAGCTCTTT |
| 285 | CAATGAAGATACAGCTGTTG |
| 286 | ACTGGTACAACTTCAGGGAC |
| 287 | CTTGTACTGGTACAACTTCA |
| 288 | ACTTGTACTGGTACAACTTC |
| 289 | TTGGCAGTTTCTACTTGTAC |
| 290 | TACCTGATAACTTCTCTACT |
| 291 | AGCCGAGTAGAGAAGTTATC |
| 292 | AGCTGCATGTTTGAGCCTGA |
| 293 | GCTGCATGTTTGAGCCTGAA |
| 294 | AAGCTGCAGGCATTCCCTTC |
| 295 | GGTACTGTCCGTCAAGCTGC |
| 296 | AGGGAATGCCTGCAGCTTGA |
| 297 | CTTGACGGACAGTACCGCAG |
| 298 | CGCCAGCACGTGCTCCTCTG |
| 299 | TACCGCAGAGGAGCACGTGC |
| 300 | AGAGGAGCACGTGCTGGCGC |
| 301 | GGAGCACGTGCTGGCGCTGG |
| 302 | AGCACGCAGCTGACGAAGCT |
| 303 | GCACGCAGCTGACGAAGCTC |
| 304 | CAGCTGACGAAGCTCGGGAC |
| 305 | AAGCTCGGGACAGGATCAAC |
| 306 | CCTTGCCGCCTGGGAGGAAC |
| 307 | AGGATCAACCGGTTCCTCCC |
| 308 | ATCAACCGGTTCCTCCCAGG |
| 309 | GCACTACCTTGCCGCCTGGG |
| 310 | AGAGCACTACCTTGCCGCCT |
| 311 | CCGGTTCCTCCCAGGCGGCA |
| 312 | TCCTCTTCAGATAGCCCATC |
| 313 | ATGGGCTATCTGAAGAGGAA |
| 314 | GGGCTATCTGAAGAGGAACG |
| 315 | TGGGCTATCTGAAGAGGAAC |
| 316 | TATCTGAAGAGGAACGGGGA |
| 317 | ATCTGAAGAGGAACGGGGAC |
| 318 | TGTTGACCACGCTGTAGAGC |
| 319 | GCTCTACAGCGTGGTCAACA |
| 320 | CGGGAGCCTGCTCTACAGCG |
| 321 | CGTGGTCAACACGGCCGAGC |
| 322 | CCCACCATCAGCGTCCGGCT |
| 323 | ACGGCCGAGCCGGACGCTGA |
| 324 | GGGCACCCACCATCAGCGTC |
| 325 | GCCGAGCCGGACGCTGATGG |
| 326 | CCATGTCCGTGTTGCAGAGG |
| 327 | CCGAGCCGGACGCTGATGGT |
| 328 | CGAGCTCAAGTCCACCGGGT |
| 329 | GCGAGCTCAAGTCCACCGGG |
| 330 | AGAGCGAGCTCAAGTCCACC |
| 331 | GAGAGCGAGCTCAAGTCCAC |
| 332 | GAAGCCTGGGAGTAGCTTAC |
| 333 | CTCTCCAGTAAGCTACTCCC |
| 334 | AGCCCAGCGTGGTGAAGCCT |
| 335 | AAGCCCAGCGTGGTGAAGCC |
| 336 | ACTCCCAGGCTTCACCACGC |
| 337 | CTCCCAGGCTTCACCACGCT |
| 338 | CTCGTCTTTGAAGCCCAGCG |
| 339 | CACTGGAGAGAAAGGTGACT |
| 340 | GCACTGGAGAGAAAGGTGAC |
| 341 | AGTAGTGGCACTGGAGAGAA |
| 342 | CGAAAGCGCAGTAGTGGCAC |
| 343 | CTGCATCGAAAGCGCAGTAG |
| 344 | ATGCAGAATAATTCAGTATT |
| 345 | AGTATTTGGCGACTTGAAGT |
| 346 | CGACTTGAAGTCGGACGAGA |
| 347 | GAGCTGCTCTACTCAGCCTA |
| 348 | CACGCCTGTCTCATCTCCGT |
| 349 | TCAGCCTACGGAGATGAGAC |
| 350 | CAGGCGTGCAGTGTGCGCTG |
| 351 | CCGCGGCCCCTCTAGCCTGC |
| 352 | CATCCTTCACAAACTCCTGC |
| 353 | TAGCCTGCAGGAGTTTGTGA |
| 354 | CAGGAGTTTGTGAAGGATGC |
| 355 | AGGAGTTTGTGAAGGATGCT |
| 356 | TGGGAGCTACAGCAAGAAAG |
| 357 | GAGCTACAGCAAGAAAGTGG |
| 358 | GAAAGTGGTGGACGACCTCC |
| 359 | CGCCTGTGATCTGGTCCAGG |
| 360 | CTCCGCCTGTGATCTGGTCC |
| 361 | GACCTCCTGGACCAGATCAC |
| 362 | CTCCTGGACCAGATCACAGG |
| 363 | GCTGGAAGAGCGTCCTAGAG |
| 364 | TGCAGCCCACCTGCTTCAGC |
| 365 | GACGCTCTTCCAGCTGAAGC |
| 366 | CTCTTCCAGCTGAAGCAGGT |
| 367 | GCTCTTCCAGCTGAAGCAGG |
| 368 | CCTCCAGATGAAGCCAAGGT |
| 369 | GCTTCATCTGGAGGCTTCAT |
| 370 | GGCTTCATCTGGAGGCTTCA |
| 371 | CTTACCTTGGCTTCATCTGG |
| 372 | AAACTTACCTTGGCTTCATC |
| 373 | GAAGCCTCCAGATGAAGCCA |
| 374 | TCCTAGGGTGTCCCCAACCT |
| 375 | CCTAGGGTGTCCCCAACCTG |
| 376 | GTGTCTGTCTCCACAGGTTG |
| 377 | TGTGTCTGTCTCCACAGGTT |
| 378 | CCACAGGTTGGGGACACCCT |
| 379 | AGAGCTGCTGCTGTCTCCTA |
| 380 | CAGAGCTGCTGCTGTCTCCT |
| 381 | AGACAGCAGCAGCTCTGTTC |
| 382 | ATCCACAGAAACGTCGGGAT |
| 383 | GAGATATCCACAGAAACGTC |
| 384 | GGAGATATCCACAGAAACGT |
| 385 | GTCCTATCCCGACGTTTCTG |
| 386 | TCTCCATGCTCAGCTCTCTG |
| 387 | CTCACCCAGAGAGCTGAGCA |
| 388 | ATCTCCATGCTCAGCTCTCT |
| 389 | TATCTCCATGCTCAGCTCTC |
| 390 | ATGTCCTGTTTACACAGGGA |
| 391 | TTACACAGGGAAGGTGAAGA |
| 392 | AGTTCAAATGGCTGTCGTCA |
| 393 | TGACGACAGCCATTTGAACT |
| 394 | AAGTTCAAATGGCTGTCGTC |
| 395 | TCGTCTCATCCAAGTTCAAA |
| 396 | TGAGACGACGAAGCTCCTGC |
| 397 | GTGCTTCGTGCAGGTCCTGC |
| 398 | GCAGGACCTGCACGAAGCAC |
| 399 | GCTCCGCCTGTGCTTCGTGC |
| 400 | GGACCTGCACGAAGCACAGG |
| 401 | CACGAAGCACAGGCGGAGCG |
| 402 | AGGCGGAGCGCGGCGGCTCT |
| 403 | AGGGAGCTGAGGTTGGACGA |
| 404 | GTTGGACAGGGAGCTGAGGT |
| 405 | AGGCGTTGGACAGGGAGCTG |
| 406 | CCCTCTCGGAGGCGTTGGAC |
| 407 | CCTCTCGGAGGCGTTGGACA |
| 408 | CTGGTCCCTCTCGGAGGCGT |
| 409 | CCCTGTCCAACGCCTCCGAG |
| 410 | CCTGTCCAACGCCTCCGAGA |
| 411 | GTGGTGCTGGTCCCTCTCGG |
| 412 | CAGGTGGTGCTGGTCCCTCT |
| 413 | GCATCTCACCCAGGTGGTGC |
| 414 | CGAGAGGGACCAGCACCACC |
| 415 | GAGAGGGACCAGCACCACCT |
| 416 | GTGGGGGCATCTCACCCAGG |
| 417 | CCCCGACACTCAGGCGAGAA |
| 418 | TCCCCGACACTCAGGCGAGA |
| 419 | AGCCCTTCTCGCCTGAGTGT |
| 420 | CTGGCTGCTCCCCGACACTC |
| 421 | CCCTTCTCGCCTGAGTGTCG |
| 422 | GCCCTTCTCGCCTGAGTGTC |
| 423 | TAGGGGTCGTGGGTGACGTC |
| 424 | AAGAAACTCATAGGGGTCGT |
| 425 | GAAGAAACTCATAGGGGTCG |
| 426 | GAGACTGAAGAAACTCATAG |
| 427 | GGAGACTGAAGAAACTCATA |
| 428 | TGGAGACTGAAGAAACTCAT |
| 429 | TCTTCAGTCTCCAGAGCCTG |
| 430 | TTGGCAGAGGCCGCAGGCTC |
| 431 | TAGGTCTTGGCAGAGGCCGC |
| 432 | CTAGAGTTAGGTCTTGGCAG |
| 433 | GGTGGTCTAGAGTTAGGTCT |

TABLE 3
Control sgRNA Library

| SEQ | | | |
|---|---|---|---|
| ID | | | |
| NO. | gRNA Label | Gene | Nucleic Acid Sequence |
| 434 | 1\|sg_Non_Targeting_Human_ | Non_Targeting_Human | GTAGCGAACGTGTCCGGCGT |
| | 0001\|Non_Targeting_Human | | |
| 435 | 1\|sg_Non_Targeting_Human_ | Non_Targeting_Human | GACCGGAACGATCTCGCGTA |
| | 0002\|Non_Targeting_Human | | |
| 436 | 1\|sg_Non_Targeting_Human_ | Non_Targeting_Human | GGCAGTCGTTCGGTTGATAT |
| | 0003\|Non_Targeting_Human | | |
| 437 | 1\|sg_Non_Targeting_Human_ | Non_Targeting_Human | GCTTGAGCACATACGCGAAT |
| | 0004\|Non_Targeting_Human | | |
| 438 | 1\|sg_Non_Targeting_Human_ | Non_Targeting_Human | GTGGTAGAATAACGTATTAC |
| | 0005\|Non_Targeting_Human | | |
| 439 | 1\|sg_Non_Targeting_Human_ | Non_Targeting_Human | GTCATACATGGATAAGGCTA |
| | 0006\|Non_Targeting_Human | | |
| 440 | 1\|sg_Non_Targeting_Human_ | Non_Targeting_Human | GATACACGAAGCATCACTAG |
| | 0007\|Non_Targeting_Human | | |
| 441 | 1\|sg_Non_Targeting_Human_ | Non_Targeting_Human | GAACGTTGGCACTACTTCAC |
| | 0008\|Non_Targeting_Human | | |
| 442 | 1\|sg_Non_Targeting_Human_ | Non_Targeting_Human | GATCCATGTAATGCGTTCGA |
| | 0009\|Non_Targeting_Human | | |
| 443 | 1\|sg_Non_Targeting_Human_ | Non_Targeting_Human | GTCGTGAAGTGCATTCGATC |
| | 0010\|Non_Targeting_Human | | |
| 444 | 1\|sg_Non_Targeting_Human_ | Non_Targeting_Human | GTTCGACTCGCGTGACCGTA |
| | 0011\|Non_Targeting_Human | | |
| 445 | 1\|sg_Non_Targeting_Human_ | Non_Targeting_Human | GAATCTACCGCAGCGGTTCG |
| | 0012\|Non_Targeting_Human | | |
| 446 | 1\|sg_Non_Targeting_Human_ | Non_Targeting_Human | GAAGTGACGTCGATTCGATA |
| | 0013\|Non_Targeting_Human | | |
| 447 | 1\|sg_Non_Targeting_Human_ | Non_Targeting_Human | GCGGTGTATGACAACCGCCG |
| | 0014\|Non_Targeting_Human | | |
| 448 | 1\|sg_Non_Targeting_Human_ | Non_Targeting_Human | GTACCGCGCCTGAAGTTCGC |
| | 0015\|Non_Targeting_Human | | |
| 449 | 1\|sg_Non_Targeting_Human_ | Non_Targeting_Human | GCAGCTCGTGTGTCGTACTC |
| | 0016\|Non_Targeting_Human | | |
| 450 | 1\|sg_Non_Targeting_Human_ | Non_Targeting_Human | GCGCCTTAAGAGTACTCATC |
| | 0017\|Non_Targeting_Human | | |
| 451 | 1\|sg_Non_Targeting_Human_ | Non_Targeting_Human | GAGTGTCGTCGTTGCTCCTA |
| | 0018\|Non_Targeting_Human | | |
| 452 | 1\|sg_Non_Targeting_Human_ | Non_Targeting_Human | GCAGCTCGACCTCAAGCCGT |
| | 0019\|Non_Targeting_Human | | |
| 453 | 1\|sg_Non_Targeting_Human_ | Non_Targeting_Human | GTATCCTGACCTACGCGCTG |
| | 0020\|Non_Targeting_Human | | |
| 454 | 1\|sg_Non_Targeting_Human_ | Non_Targeting_Human | GTGTATCTCAGCACGCTAAC |
| | 0021\|Non_Targeting_Human | | |
| 455 | 1\|sg_Non_Targeting_Human_ | Non_Targeting_Human | GTCGTCATACAACGGCAACG |
| | 0022\|Non_Targeting_Human | | |
| 456 | 1\|sg_Non_Targeting_Human_ | Non_Targeting_Human | GTCGTGCGCTTCCGGCGGTA |
| | 0023\|Non_Targeting_Human | | |
| 457 | 1\|sg_Non_Targeting_Human_ | Non_Targeting_Human | GCGGTCCTCAGTAAGCGCGT |
| | 0024\|Non_Targeting_Human | | |
| 458 | 1\|sg_Non_Targeting_Human_ | Non_Targeting_Human | GCTCTGCTGCGGAAGGATTC |
| | 0025\|Non_Targeting_Human | | |
| 459 | 1\|sg_Non_Targeting_Human_ | Non_Targeting_Human | GCATGGAGGAGCGTCGCAGA |
| | 0026\|Non_Targeting_Human | | |
| 460 | 1\|sg_Non_Targeting_Human_ | Non_Targeting_Human | GTAGCGCGCGTAGGAGTGGC |
| | 0027\|Non_Targeting_Human | | |
| 461 | 1\|sg_Non_Targeting_Human_ | Non_Targeting_Human | GATCACCTGCATTCGTACAC |
| | 0028\|Non_Targeting_Human | | |
| 462 | 1\|sg_Non_Targeting_Human_ | Non_Targeting_Human | GCACACCTAGATATCGAATG |
| | 0029\|Non_Targeting_Human | | |
| 463 | 1\|sg_Non_Targeting_Human_ | Non_Targeting_Human | GTTGATCAACGCGCTTCGCG |
| | 0030\|Non_Targeting_Human | | |
| 464 | 1\|sg_Non_Targeting_Human_ | Non_Targeting_Human | GCGTCTCACTCACTCCATCG |
| | 0031\|Non_Targeting_Human | | |
| 465 | 1\|sg_Non_Targeting_Human_ | Non_Targeting_Human | GCCGACCAACGTCAGCGGTA |
| | 0032\|Non_Targeting_Human | | |
| 466 | 1\|sg_Non_Targeting_Human_ | Non_Targeting_Human | GGATACGGTGCGTCAATCTA |
| | 0033\|Non_Targeting_Human | | |
| 467 | 1\|sg_Non_Targeting_Human_ | Non_Targeting_Human | GAATCCAGTGGCGGCGACAA |
| | 0034\|Non_Targeting_Human | | |
| 468 | 1\|sg_Non_Targeting_Human_ | Non_Targeting_Human | GCACTGTCAGTGCAACGATA |
| | 0035\|Non_Targeting_Human | | |
| 469 | 1\|sg_Non_Targeting_Human_ | Non_Targeting_Human | GCGATCCTCAAGTATGCTCA |
| | 0036\|Non_Targeting_Human | | |
| 470 | 1\|sg_Non_Targeting_Human_ | Non_Targeting_Human | GCTAATATCGACACGGCCGC |
| | 0037\|Non_Targeting_Human | | |
| 471 | 1\|sg_Non_Targeting_Human_ | Non_Targeting_Human | GGAGATGCATCGAAGTCGAT |
| | 0038\|Non_Targeting_Human | | |
| 472 | 1\|sg_Non_Targeting_Human_ | Non_Targeting_Human | GGATGCACTCCATCTCGTCT |
| | 0039\|Non_Targeting_Human | | |
| 473 | 1\|sg_Non_Targeting_Human_ | Non_Targeting_Human | GTGCCGAGTAATAACGCGAG |
| | 0040\|Non_Targeting_Human | | |
| 474 | 1\|sg_Non_Targeting_Human_ | Non_Targeting_Human | GAGATTCCGATGTAACGTAC |
| | 0041\|Non_Targeting_Human | | |
| 475 | 1\|sg_Non_Targeting_Human_ | Non_Targeting_Human | GTCGTCACGAGCAGGATTGC |
| | 0042\|Non_Targeting_Human | | |
| 476 | 1\|sg_Non_Targeting_Human_ | Non_Targeting_Human | GCGTTAGTCACTTAGCTCGA |
| | 0043\|Non_Targeting_Human | | |
| 477 | 1\|sg_Non_Targeting_Human_ | Non_Targeting_Human | GTTCACACGGTGTCGGATAG |
| | 0044\|Non_Targeting_Human | | |
| 478 | 1\|sg_Non_Targeting_Human_ | Non_Targeting_Human | GGATAGGTGACCTTAGTACG |
| | 0045\|Non_Targeting_Human | | |
| 479 | 1\|sg_Non_Targeting_Human_ | Non_Targeting_Human | GTATGAGTCAAGCTAATGCG |
| | 0046\|Non_Targeting_Human | | |
| 480 | 1\|sg_Non_Targeting_Human_ | Non_Targeting_Human | GCAACTATTGGAATACGTGA |
| | 0047\|Non_Targeting_Human | | |
| 481 | 1\|sg_Non_Targeting_Human_ | Non_Targeting_Human | GTTACCTTCGCTCGTCTATA |
| | 0048\|Non_Targeting_Human | | |
| 482 | 1\|sg_Non_Targeting_Human_ | Non_Targeting_Human | GTACCGAGCACCACAGGCCG |
| | 0049\|Non_Targeting_Human | | |
| 483 | 1\|sg_Non_Targeting_Human_ | Non_Targeting_Human | GTCAGCCATCGGATAGAGAT |
| | 0050\|Non_Targeting_Human | | |
| 484 | 1\|sg_Non_Targeting_Human_ | Non_Targeting_Human | GTACGGCACTCCTAGCCGCT |
| | 0051\|Non_Targeting_Human | | |
| 485 | 1\|sg_Non_Targeting_Human_ | Non_Targeting_Human | GGTCCTGTCGTATGCTTGCA |
| | 0052\|Non_Targeting_Human | | |
| 486 | 1\|sg_Non_Targeting_Human_ | Non_Targeting_Human | GCCGCAATATATGCGGTAAG |
| | 0053\|Non_Targeting_Human | | |
| 487 | 1\|sg_Non_Targeting_Human_ | Non_Targeting_Human | GCGCACGTATAATCCTGCGT |
| | 0054\|Non_Targeting_Human | | |
| 488 | 1\|sg_Non_Targeting_Human_ | Non_Targeting_Human | GTGCACAACACGATCCACGA |
| | 0055\|Non_Targeting_Human | | |
| 489 | 1\|sg_Non_Targeting_Human_ | Non_Targeting_Human | GCACAATGTTGACGTAAGTG |
| | 0056\|Non_Targeting_Human | | |
| 490 | 1\|sg_Non_Targeting_Human_ | Non_Targeting_Human | GTAAGATGCTGCTCACCGTG |
| | 0057\|Non_Targeting_Human | | |
| 491 | 1\|sg_Non_Targeting_Human_ | Non_Targeting_Human | GTCGGTGATCCAACGTATCG |
| | 0058\|Non_Targeting_Human | | |
| 492 | 1\|sg_Non_Targeting_Human_ | Non_Targeting_Human | GAGCTAGTAGGACGCAAGAC |
| | 0059\|Non_Targeting_Human | | |
| 493 | 1\|sg_Non_Targeting_Human_ | Non_Targeting_Human | GTACGTGGAAGCTTGTGGCC |
| | 0060\|Non_Targeting_Human | | |
| 494 | 1\|sg_Non_Targeting_Human_ | Non_Targeting_Human | GAGAACTGCCAGTTCTCGAT |
| | 0061\|Non_Targeting_Human | | |
| 495 | 1\|sg_Non_Targeting_Human_ | Non_Targeting_Human | GCCATTCGGCGCGGCACTTC |
| | 0062\|Non_Targeting_Human | | |
| 496 | 1\|sg_Non_Targeting_Human_ | Non_Targeting_Human | GCACACGACCAATCCGCTTC |
| | 0063\|Non_Targeting_Human | | |
| 497 | 1\|sg_Non_Targeting_Human_ | Non_Targeting_Human | GAGGTGATCGATTAAGTACA |
| | 0064\|Non_Targeting_Human | | |
| 498 | 1\|sg_Non_Targeting_Human_ | Non_Targeting_Human | GTCACTCGCAGACGCCTAAC |
| | 0065\|Non_Targeting_Human | | |
| 499 | 1\|sg_Non_Targeting_Human_ | Non_Targeting_Human | GCGCTACGGAATCATACGTT |
| | 0066\|Non_Targeting_Human | | |
| 500 | 1\|sg_Non_Targeting_Human_ | Non_Targeting_Human | GGTAGGACCTCACGGCGCGC |
| | 0067\|Non_Targeting_Human | | |
| 501 | 1\|sg_Non_Targeting_Human_ | Non_Targeting_Human | GAACTGCATCTTGTTGTAGT |
| | 0068\|Non_Targeting_Human | | |
| 502 | 1\|sg_Non_Targeting_Human_ | Non_Targeting_Human | GATCCTGATCCGGCGGCGCG |
| | 0069\|Non_Targeting_Human | | |
| 503 | 1\|sg_Non_Targeting_Human_ | Non_Targeting_Human | GGTATGCGCGATCCTGAGTT |
| | 0070\|Non_Targeting_Human | | |
| 504 | 1\|sg_Non_Targeting_Human_ | Non_Targeting_Human | GCGGAGCTAGAGAGCGGTCA |
| | 0071\|Non_Targeting_Human | | |
| 505 | 1\|sg_Non_Targeting_Human_ | Non_Targeting_Human | GAATGGCAATTACGGCTGAT |
| | 0072\|Non_Targeting_Human | | |
| 506 | 1\|sg_Non_Targeting_Human_ | Non_Targeting_Human | GTATGGTGAGTAGTCGCTTG |
| | 0073\|Non_Targeting_Human | | |
| 507 | 1\|sg_Non_Targeting_Human_ | Non_Targeting_Human | GTGTAATTGCGTCTAGTCGG |
| | 0074\|Non_Targeting_Human | | |
| 508 | 1\|sg_Non_Targeting_Human_ | Non_Targeting_Human | GGTCCTGGCGAGGAGCCTTG |
| | 0075\|Non_Targeting_Human | | |
| 509 | 1\|sg_Non_Targeting_Human_ | Non_Targeting_Human | GAAGATAAGTCGCTGTCTCG |
| | 0076\|Non_Targeting_Human | | |
| 510 | 1\|sg_Non_Targeting_Human_ | Non_Targeting_Human | GTCGGCGTTCTGTTGTGACT |
| | 0077\|Non_Targeting_Human | | |
| 511 | 1\|sg_Non_Targeting_Human_ | Non_Targeting_Human | GAGGCAAGCCGTTAGGTGTA |
| | 0078\|Non_Targeting_Human | | |
| 512 | 1\|sg_Non_Targeting_Human_ | Non_Targeting_Human | GCGGATCCAGATCTCATTCG |
| | 0079\|Non_Targeting_Human | | |
| 513 | 1\|sg_Non_Targeting_Human_ | Non_Targeting_Human | GGAACATAGGAGCACGTAGT |
| | 0080\|Non_Targeting_Human | | |
| 514 | 1\|sg_Non_Targeting_Human_ | Non_Targeting_Human | GTCATCATTATGGCGTAAGG |
| | 0081\|Non_Targeting_Human | | |
| 515 | 1\|sg_Non_Targeting_Human_ | Non_Targeting_Human | GCGACTAGCGCCATGAGCGG |
| | 0082\|Non_Targeting_Human | | |
| 516 | 1\|sg_Non_Targeting_Human_ | Non_Targeting_Human | GGCGAAGTTCGACATGACAC |
| | 0083\|Non_Targeting_Human | | |
| 517 | 1\|sg_Non_Targeting_Human_ | Non_Targeting_Human | GCTGTCGTGTGGAGGCTATG |
| | 0084\|Non_Targeting_Human | | |
| 518 | 1\|sg_Non_Targeting_Human_ | Non_Targeting_Human | GCGGAGAGCATTGACCTCAT |
| | 0085\|Non_Targeting_Human | | |
| 519 | 1\|sg_Non_Targeting_Human_ | Non_Targeting_Human | GACTAATGGACCAAGTCAGT |
| | 0086\|Non_Targeting_Human | | |
| 520 | 1\|sg_Non_Targeting_Human_ | Non_Targeting_Human | GCGGATTAGAGGTAATGCGG |
| | 0087\|Non_Targeting_Human | | |
| 521 | 1\|sg_Non_Targeting_Human_ | Non_Targeting_Human | GCCGACGGCAATCAGTACGC |
| | 0088\|Non_Targeting_Human | | |
| 522 | 1\|sg_Non_Targeting_Human_ | Non_Targeting_Human | GTAACCTCTCGAGCGATAGA |
| | 0089\|Non_Targeting_Human | | |
| 523 | 1\|sg_Non_Targeting_Human_ | Non_Targeting_Human | GACTTGTATGTGGCTTACGG |
| | 0090\|Non_Targeting_Human | | |
| 524 | 1\|sg_Non_Targeting_Human_ | Non_Targeting_Human | GTCACTGTGGTCGAACATGT |
| | 0091\|Non_Targeting_Human | | |
| 525 | 1\|sg_Non_Targeting_Human_ | Non_Targeting_Human | GTACTCCAATCCGCGATGAC |
| | 0092\|Non_Targeting_Human | | |
| 526 | 1\|sg_Non_Targeting_Human_ | Non_Targeting_Human | GCGTTGGCACGATGTTACGG |
| | 0093\|Non_Targeting_Human | | |
| 527 | 1\|sg_Non_Targeting_Human_ | Non_Targeting_Human | GAACCAGCCGGCTAGTATGA |
| | 0094\|Non_Targeting_Human | | |
| 528 | 1\|sg_Non_Targeting_Human_ | Non_Targeting_Human | GTATACTAGCTAACCACACG |
| | 0095\|Non_Targeting_Human | | |
| 529 | 1\|sg_Non_Targeting_Human_ | Non_Targeting_Human | GAATCGGAATAGTTGATTCG |
| | 0096\|Non_Targeting_Human | | |
| 530 | 1\|sg_Non_Targeting Human_ | Non_Targeting_Human | GAGCACTTGCATGAGGCGGT |
| | 0097\|Non_Targeting Human | | |
| 531 | 1\|sg_Non_Targeting_Human_ | Non_Targeting_Human | GAACGGCGATGAAGCCAGCC |
| | 0098\|Non_Targeting_Human | | |
| 532 | 1\|sg_Non_Targeting_Human_ | Non_Targeting_Human | GCAACCGAGATGAGAGGTTC |
| | 0099\|Non Targeting_Human | | |
| 533 | 1\|sg_Non_Targeting_Human_ | Non_Targeting_Human | GCAAGATCAATATGCGTGAT |
| | 0100\|Non_Targeting_Human | | |
| 534 | 1\|sg_Non_Targeting_Human_ | Non_Targeting_Human | ACGGAGGCTAAGCGTCGCAA |
| | GA_0101\|Non_Targeting_Human | | |
| 535 | 1\|sg_Non_Targeting_Human_ | Non_Targeting_Human | CGCTTCCGCGGCCCGTTCAA |
| | GA_0102\|Non_Targeting_Human | | |
| 536 | 1\|sg_Non_Targeting_Human_ | Non_Targeting_Human | ATCGTTTCCGCTTAACGGCG |
| | GA_0103\|Non_Targeting_Human | | |
| 537 | 1\|sg_Non_Targeting_Human_ | Non_Targeting_Human | GTAGGCGCGCCGCTCTCTAC |
| | GA_0104\|Non_Targeting_Human | | |
| 538 | 1\|sg_Non_Targeting_Human_ | Non_Targeting_Human | CCATATCGGGGCGAGACATG |
| | GA_0105\|Non_Targeting_Human | | |
| 539 | 1\|sg_Non_Targeting_Human_ | Non_Targeting_Human | TACTAACGCCGCTCCTACAG |
| | GA_0106\|Non_Targeting_Human | | |
| 540 | 1\|sg_Non_Targeting_Human_ | Non_Targeting_Human | TGAGGATCATGTCGAGCGCC |
| | GA_0107\|Non_Targeting_Human | | |
| 541 | 1\|sg_Non_Targeting_Human_ | Non_Targeting_Human | GGGCCCGCATAGGATATCGC |
| | GA_0108\|Non_Targeting_Human | | |
| 542 | 1\|sg_Non_Targeting_Human_ | Non_Targeting_Human | TAGACAACCGCGGAGAATGC |
| | GA_0109\|Non_Targeting_Human | | |
| 543 | 1\|sg_Non_Targeting_Human_ | Non_Targeting_Human | ACGGGCGGCTATCGCTGACT |
| | GA_0110\|Non_Targeting_Human | | |
| 544 | 1\|sg_Non_Targeting_Human_ | Non_Targeting_Human | CGCGGAAATTTTACCGACGA |
| | GA_0111\|Non_Targeting_Human | | |
| 545 | 1\|sg_Non_Targeting_Human_ | Non_Targeting_Human | CTTACAATCGTCGGTCCAAT |
| | GA_0112\|Non_Targeting_Human | | |
| 546 | 1\|sg_Non_Targeting_Human_ | Non_Targeting_Human | GCGTGCGTCCCGGGTTACCC |
| | GA_0113\|Non_Targeting_Human | | |
| 547 | 1\|sg_Non_Targeting_Human_ | Non_Targeting_Human | CGGAGTAACAAGCGGACGGA |
| | GA_0114\|Non_Targeting_Human | | |
| 548 | 1\|sg_Non_Targeting_Human_ | Non_Targeting_Human | CGAGTGTTATACGCACCGTT |
| | GA_0115\|Non_Targeting_Human | | |
| 549 | 1\|sg_Non_Targeting_Human_ | Non_Targeting_Human | CGACTAACCGGAAACTTTTT |
| | GA_0116\|Non_Targeting_Human | | |
| 550 | 1\|sg_Non_Targeting_Human_ | Non_Targeting_Human | CAACGGGTTCTCCCGGCTAC |
| | GA_0117\|Non_Targeting_Human | | |
| 551 | 1\|sg_Non_Targeting_Human_ | Non_Targeting_Human | CAGGAGTCGCCGATACGCGT |
| | GA_0118\|Non_Targeting_Human | | |
| 552 | 1\|sg_Non_Targeting_Human_ | Non_Targeting_Human | TTCACGTCGTCTCGCGACCA |
| | GA_0119\|Non_Targeting_Human | | |
| 553 | 1\|sg_Non_Targeting_Human_ | Non_Targeting_Human | GTGTCGGATTCCGCCGCTTA |
| | GA_0120\|Non_Targeting_Human | | |
| 554 | 1\|sg_Non_Targeting_Human_ | Non_Targeting_Human | CACGAACTCACACCGCGCGA |
| | GA_0121\|Non_Targeting_Human | | |
| 555 | 1\|sg_Non_Targeting_Human_ | Non_Targeting_Human | CGCTAGTACGCTCCTCTATA |
| | GA_0122\|Non_Targeting_Human | | |
| 556 | 1\|sg_Non_Targeting_Human_ | Non_Targeting_Human | TCGCGCTTGGGTTATACGCT |
| | GA_0123\|Non_Targeting_Human | | |
| 557 | 1\|sg_Non_Targeting_Human_ | Non_Targeting_Human | CTATCTCGAGTGGTAATGCG |
| | GA_0124\|Non_Targeting_Human | | |
| 558 | 1\|sg_Non_Targeting_Human_ | Non_Targeting_Human | AATCGACTCGAACTTCGTGT |
| | GA_0125\|Non_Targeting_Human | | |
| 559 | 1\|sg_Non_Targeting_Human_ | Non_Targeting_Human | CCCGATGGACTATACCGAAC |
| | GA_0126\|Non_Targeting_Human | | |
| 560 | 1\|sg_Non_Targeting_Human_ | Non_Targeting_Human | ACGTTCGAGTACGACCAGCT |
| | GA_0127\|Non_Targeting Human | | |
| 561 | 1\|sg_Non_Targeting_Human_ | Non_Targeting_Human | CGCGACGACTCAACCTAGTC |
| | GA_0128\|Non_Targeting_Human | | |
| 562 | 1\|sg_Non_Targeting_Human_ | Non_Targeting_Human | GGTCACCGATCGAGAGCTAG |
| | GA_0129\|Non_Targeting_Human | | |
| 563 | 1\|sg_Non_Targeting_Human_ | Non_Targeting_Human | CTCAACCGACCGTATGGTCA |
| | GA_0130\|Non_Targeting_Human | | |
| 564 | 1\|sg_Non_Targeting_Human_ | Non_Targeting_Human | CGTATTCGACTCTCAACGCG |
| | GA_0131\|Non_Targeting_Human | | |
| 565 | 1\|sg_Non_Targeting_Human_ | Non_Targeting_Human | CTAGCCGCCCAGATCGAGCC |
| | GA_0132\|Non_Targeting_Human | | |
| 566 | 1\|sg_Non_Targeting_Human_ | Non_Targeting_Human | GAATCGACCGACACTAATGT |
| | GA_0133\|Non_Targeting_Human | | |
| 567 | 1\|sg_Non_Targeting_Human_ | Non_Targeting_Human | ACTTCAGTTCGGCGTAGTCA |
| | GA_0134\|Non_Targeting_Human | | |
| 568 | 1\|sg_Non_Targeting_Human_ | Non_Targeting_Human | GTGCGATGTCGCTTCAACGT |
| | GA_0135\|Non_Targeting_Human | | |
| 569 | 1\|sg_Non_Targeting_Human_ | Non_Targeting_Human | CGCCTAATTTCCGGATCAAT |
| | GA_0136\|Non_Targeting_Human | | |
| 570 | 1\|sg_Non_Targeting_Human_ | Non_Targeting_Human | CGTGGCCGGAACCGTCATAG |
| | GA_0137\|Non_Targeting_Human | | |
| 571 | 1\|sg_Non_Targeting_Human_ | Non_Targeting_Human | ACCCTCCGAATCGTAACGGA |
| | GA_0138\|Non_Targeting_Human | | |
| 572 | 1\|sg_Non_Targeting_Human_ | Non_Targeting_Human | AAACGGTACGACAGCGTGTG |
| | GA_0139\|Non_Targeting_Human | | |
| 573 | 1\|sg_Non_Targeting_Human_ | Non_Targeting_Human | ACATAGTCGACGGCTCGATT |
| | GA_0140\|Non_Targeting_Human | | |
| 574 | 1\|sg_Non_Targeting_Human_ | Non_Targeting_Human | GATGGCGCTTCAGTCGTCGG |
| | GA_0141\|Non_Targeting_Human | | |
| 575 | 1\|sg_Non_Targeting_Human_ | Non_Targeting_Human | ATAATCCGGAAACGCTCGAC |
| | GA_0142\|Non_Targeting_Human | | |
| 576 | 1\|sg_Non_Targeting_Human_ | Non_Targeting_Human | CGCCGGGCTGACAATTAACG |
| | GA_0143\|Non_Targeting_Human | | |
| 577 | 1\|sg_Non_Targeting_Human_ | Non_Targeting_Human | CGTCGCCATATGCCGGTGGC |
| | GA_0144\|Non_Targeting_Human | | |
| 578 | 1\|sg_Non_Targeting_Human_ | Non_Targeting_Human | CGGGCCTATAACACCATCGA |
| | GA_0145\|Non_Targeting_Human | | |
| 579 | 1\|sg_Non_Targeting_Human_ | Non_Targeting_Human | CGCCGTTCCGAGATACTTGA |
| | GA_0146\|Non_Targeting_Human | | |
| 580 | 1\|sg_Non_Targeting_Human_ | Non_Targeting_Human | CGGGACGTCGCGAAAATGTA |
| | GA_0147\|Non_Targeting_Human | | |
| 581 | 1\|sg_Non_Targeting_Human_ | Non_Targeting_Human | TCGGCATACGGGACACACGC |
| | GA_0148\|Non_Targeting_Human | | |
| 582 | 1\|sg_Non_Targeting_Human_ | Non_Targeting_Human | AGCTCCATCGCCGCGATAAT |
| | GA_0149\|Non_Targeting_Human | | |
| 583 | 1\|sg_Non_Targeting_Human_ | Non_Targeting_Human | ATCGTATCATCAGCTAGCGC |
| | GA_0150\|Non_Targeting_Human | | |
| 584 | 1\|sg_Non_Targeting_Human_ | Non_Targeting_Human | TCGATCGAGGTTGCATTCGG |
| | GA_0151\|Non_Targeting_Human | | |
| 585 | 1\|sg_Non_Targeting_Human_ | Non_Targeting_Human | CTCGACAGTTCGTCCCGAGC |
| | GA_0152\|Non_Targeting_Human | | |
| 586 | 1\|sg_Non_Targeting_Human_ | Non_Targeting_Human | CGGTAGTATTAATCGCTGAC |
| | GA_0153\|Non_Targeting_Human | | |
| 587 | 1\|sg_Non_Targeting_Human_ | Non_Targeting_Human | TGAACGCGTGTTTCCTTGCA |
| | GA_0154\|Non_Targeting_Human | | |
| 588 | 1\|sg_Non_Targeting_Human_ | Non_Targeting_Human | CGACGCTAGGTAACGTAGAG |
| | GA_0155\|Non_Targeting_Human | | |
| 589 | 1\|sg_Non_Targeting_Human_ | Non_Targeting_Human | CATTGTTGAGCGGGCGCGCT |
| | GA_0156\|Non_Targeting_Human | | |
| 590 | 1\|sg_Non_Targeting_Human_ | Non_Targeting_Human | CCGCTATTGAAACCGCCCAC |
| | GA_0157\|Non_Targeting_Human | | |
| 591 | 1\|sg_Non_Targeting_Human_ | Non_Targeting_Human | AGACACGTCACCGGTCAAAA |
| | GA_0158\|Non_Targeting_Human | | |
| 592 | 1\|sg_Non_Targeting_Human_ | Non_Targeting_Human | TTTACGATCTAGCGGCGTAG |
| | GA_0159\|Non_Targeting_Human | | |
| 593 | 1\|sg_Non_Targeting_Human_ | Non_Targeting_Human | TTCGCACGATTGCACCTTGG |
| | GA_0160\|Non_Targeting_Human | | |
| 594 | 1\|sg_Non_Targeting_Human_ | Non_Targeting_Human | GGTTAGAGACTAGGCGCGCG |
| | GA_0161\|Non_Targeting_Human | | |
| 595 | 1\|sg_Non_Targeting_Human_ | Non_Targeting_Human | CCTCCGTGCTAACGCGGACG |
| | GA_0162\|Non_Targeting_Human | | |
| 596 | 1\|sg_Non_Targeting_Human_ | Non_Targeting_Human | TTATCGCGTAGTGCTGACGT |
| | GA_0163\|Non_Targeting_Human | | |
| 597 | 1\|sg_Non_Targeting_Human_ | Non_Targeting_Human | TACGCTTGCGTTTAGCGTCC |
| | GA_0164\|Non_Targeting_Human | | |
| 598 | 1\|sg_Non_Targeting_Human_ | Non_Targeting_Human | CGCGGCCCACGCGTCATCGC |
| | GA_0165\|Non_Targeting_Human | | |
| 599 | 1\|sg_Non_Targeting_Human_ | Non_Targeting_Human | AGCTCGCCATGTCGGTTCTC |
| | GA_0166\|Non_Targeting_Human | | |
| 600 | 1\|sg_Non_Targeting_Human_ | Non_Targeting_Human | AACTAGCCCGAGCAGCTTCG |
| | GA_0167\|Non_Targeting_Human | | |
| 601 | 1\|sg_Non_Targeting_Human_ | Non_Targeting_Human | CGCAAGGTGTCGGTAACCCT |
| | GA_0168\|Non_Targeting_Human | | |
| 602 | 1\|sg_Non_Targeting_Human_ | Non_Targeting_Human | CTTCGACGCCATCGTGCTCA |
| | GA_0169\|Non_Targeting_Human | | |
| 603 | 1\|sg_Non_Targeting_Human_ | Non_Targeting_Human | TCCTGGATACCGCGTGGTTA |
| | GA_0170\|Non_Targeting_Human | | |
| 604 | 1\|sg_Non_Targeting_Human_ | Non_Targeting_Human | ATAGCCGCCGCTCATTACTT |
| | GA_0171\|Non_Targeting_Human | | |
| 605 | 1\|sg_Non_Targeting_Human_ | Non_Targeting_Human | GTCGTCCGGGATTACAAAAT |
| | GA_0172\|Non_Targeting_Human | | |
| 606 | 1\|sg_Non_Targeting_Human_ | Non_Targeting_Human | TAATGCTGCACACGCCGAAT |
| | GA_0173\|Non_Targeting_Human | | |
| 607 | 1\|sg_Non_Targeting_Human_ | Non_Targeting_Human | TATCGCTTCCGATTAGTCCG |
| | GA_0174\|Non_Targeting_Human | | |
| 608 | 1\|sg_Non_Targeting_Human_ | Non_Targeting_Human | GTACCATACCGCGTACCCTT |
| | GA_0175\|Non_Targeting_Human | | |
| 609 | 1\|sg_Non_Targeting_Human_ | Non_Targeting_Human | TAAGATCCGCGGGTGGCAAC |
| | GA_0176\|Non_Targeting_Human | | |
| 610 | 1\|sg_Non_Targeting_Human_ | Non_Targeting_Human | GTAGACGTCGTGAGCTTCAC |
| | GA_0177\|Non_Targeting_Human | | |
| 611 | 1\|sg_Non_Targeting_Human_ | Non_Targeting_Human | TCGCGGACATAGGGCTCTAA |
| | GA_0178\|Non_Targeting_Human | | |
| 612 | 1\|sg_Non_Targeting_Human_ | Non_Targeting_Human | AGCGCAGATAGCGCGTATCA |
| | GA_0179\|Non_Targeting_Human | | |
| 613 | 1\|sg_Non_Targeting_Human_ | Non_Targeting_Human | GTTCGCTTCGTAACGAGGAA |
| | GA_0180\|Non_Targeting_Human | | |
| 614 | 1\|sg_Non_Targeting_Human_ | Non_Targeting_Human | GACCCCCGATAACTTTTGAC |
| | GA_0181\|Non_Targeting_Human | | |
| 615 | 1\|sg_Non_Targeting_Human_ | Non_Targeting_Human | ACGTCCATACTGTCGGCTAC |
| | GA_0182\|Non_Targeting_Human | | |
| 616 | 1\|sg_Non_Targeting_Human_ | Non_Targeting_Human | GTACCATTGCCGGCTCCCTA |
| | GA_0183\|Non_Targeting_Human | | |
| 617 | 1\|sg_Non_Targeting_Human_ | Non_Targeting_Human | TGGTTCCGTAGGTCGGTATA |
| | GA_0184\|Non_Targeting_Human | | |
| 618 | 1\|sg_Non_Targeting_Human_ | Non_Targeting_Human | TCTGGCTTGACACGACCGTT |
| | GA_0185\|Non_Targeting_Human | | |
| 619 | 1\|sg_Non_Targeting_Human_ | Non_Targeting_Human | CGCTAGGTCCGGTAAGTGCG |
| | GA_0186\|Non_Targeting_Human | | |
| 620 | 1\|sg_Non_Targeting_Human_ | Non_Targeting_Human | AGCACGTAATGTCCGTGGAT |
| | GA_0187\|Non_Targeting_Human | | |
| 621 | 1\|sg_Non_Targeting_Human_ | Non_Targeting_Human | AAGGCGCGCGAATGTGGCAG |
| | GA_0188\|Non_Targeting_Human | | |
| 622 | 1\|sg_Non_Targeting_Human_ | Non_Targeting_Human | ACTGCGGAGCGCCCAATATC |
| | GA_0189\|Non_Targeting_Human | | |
| 623 | 1\|sg_Non_Targeting_Human_ | Non_Targeting_Human | CGTCGAGTGCTCGAACTCCA |
| | GA_0190\|Non_Targeting_Human | | |
| 624 | 1\|sg_Non_Targeting_Human_ | Non_Targeting_Human | TCGCAGCGGCGTGGGATCGG |
| | GA_0191\|Non_Targeting_Human | | |
| 625 | 1\|sg_Non_Targeting_Human_ | Non_Targeting_Human | ATCTGTCCTAATTCGGATCG |
| | GA_0192\|Non_Targeting_Human | | |
| 626 | 1\|sg_Non_Targeting_Human_ | Non_Targeting_Human | TGCGGCGTAATGCTTGAAAG |
| | GA_0193\|Non_Targeting_Human | | |
| 627 | 1\|sg_Non_Targeting_Human_ | Non_Targeting_Human | CGAACTTAATCCCGTGGCAA |
| | GA_0194\|Non_Targeting_Human | | |
| 628 | 1\|sg_Non_Targeting_Human_ | Non_Targeting_Human | GCCGTGTTGCTGGATACGCC |
| | GA_0195\|Non_Targeting_Human | | |
| 629 | 1\|sg_Non_Targeting_Human_ | Non_Targeting_Human | TACCCTCCGGATACGGACTG |
| | GA_0196\|Non_Targeting_Human | | |
| 630 | 1\|sg_Non_Targeting_Human_ | Non_Targeting_Human | CCGTTGGACTATGGCGGGTC |
| | GA_0197\|Non_Targeting_Human | | |
| 631 | 1\|sg_Non_Targeting_Human_ | Non_Targeting_Human | GTACGGGGCGATCATCCACA |
| | GA_0198\|Non_Targeting_Human | | |
| 632 | 1\|sg_Non_Targeting_Human_ | Non_Targeting_Human | AAGAGTAGTAGACGCCCGGG |
| | GA_0199\|Non_Targeting_Human | | |
| 633 | 1\|sg_Non_Targeting_Human_ | Non_Targeting_Human | AAGAGCGAATCGATTTCGTG |
| | GA_0200\|Non_Targeting_Human | | |
| 634 | 3\|sg_hCDC16_CC_1\|CDC16 | CDC16 | TCAACACCAGTGCCTGACGG |
| 635 | 3\|sg_hCDC16_CC_2\|CDC16 | CDC16 | AAAGTAGCTTCACTCTCTCG |
| 636 | 3\|sg_hCDC16_CC_3\|CDC16 | CDC16 | GAGCCAACCAATAGATGTCC |
| 637 | 3\|sg_hCDC16_CC_4\|CDC16 | CDC16 | GCGCCGCCATGAACCTAGAG |
| 638 | 3\|sg_hGTF2B_CC_1\|GTF2B | GTF2B | ACAAAGGTTGGAACAGAACC |
| 639 | 3\|sg_hGTF2B CC_2\|GTF2B | GTF2B | GGTGACCGGGTTATTGATGT |
| 640 | 3\|sg_hGTF2B_CC 3\|GTF2B | GTF2B | TTAGTGGAGGACTACAGAGC |
| 641 | 3\|sg_hGTF2B_CC_4\|GTF2B | GTF2B | ACATATAGCCCGTAAAGCTG |
| 642 | 3\|sg_hHSPA5_CC_1\|HSPA5 | HSPA5 | CGTTGGCGATGATCTCCACG |
| 643 | 3\|sg_hHSPA5_CC_2\|HSPA5 | HSPA5 | TGGCCTTTTCTACCTCGCGC |
| 644 | 3\|sg_hHSPA5_CC_3\|HSPA5 | HSPA5 | AATGGAGATACTCATCTGGG |
| 645 | 3\|sg_hHSPA5_CC_4\|HSPA5 | HSPA5 | GAAGCCCGTCCAGAAAGTGT |
| 646 | 3\|sg_hHSPA9_CC_1\|HSPA9 | HSPA9 | CAATCTGAGGAACTCCACGA |
| 647 | 3\|sg_hHSPA9_CC_2\|HSPA9 | HSPA9 | AGGCTGCGGCGCCCACGAGA |
| 648 | 3\|sg_hHSPA9_CC_3\|HSPA9 | HSPA9 | ACTTTGACCAGGCCTTGCTA |
| 649 | 3\|sg_hHSPA9_CC_4\|HSPA9 | HSPA9 | ACCTTCCATAACTGCCACGC |
| 650 | 3\|sg_hPAFAH1B1_CC_1\|PAFA | PAFAH1B1 | CGAGGCGTACATACCCAAGG |
| | H1B1 | | |
| 651 | 3\|sg_hPAFAH1B1_CC_2\|PAFA | PAFAH1B1 | ATGGTACGGCCAAATCAAGA |
| | H1B1 | | |
| 652 | 3\|sg_hPAFAH1B1_CC_3\|PAFA | PAFAH1B1 | TCTTGTAATCCCATACGCGT |
| | H1B1 | | |
| 653 | 3\|sg_hPAFAH1B1_CC_4\|PAFA | PAFAH1B1 | ATTCACAGGACACAGAGAAT |
| | H1B1 | | |
| 654 | 3\|sg_hPCNA_CC_1\|PCNA | PCNA | CCAGGGCTCCATCCTCAAGA |
| 655 | 3\|sg_hPCNA_CC_2\|PCNA | PCNA | TGAGCTGCACCAAAGAGACG |
| 656 | 3\|sg_hPCNA_CC_3\|PCNA | PCNA | ATGTCTGCAGATGTACCCCT |
| 657 | 3\|sg_hPCNA_CC_4\|PCNA | PCNA | CGAAGATAACGCGGATACCT |
| 658 | 3\|sg_hPOLR2L_CC_1\|POLR2L | POLR2L | GCTGCAGGCCGAGTACACCG |
| 659 | 3\|sg_hPOLR2L_CC_2\|POLR2L | POLR2L | ACAAGTGGGAGGCTTACCTG |
| 660 | 3\|sg_hPOLR2L_CC_3\|POLR2L | POLR2L | GCAGCGTACAGGGATGATCA |
| 661 | 3\|sg_hPOLR2L_CC_4\|POLR2L | POLR2L | GCAGTAGCGCTTCAGGCCCA |
| 662 | 3\|sg_hRPL9_CC_1\|RPL9 | RPL9 | CAAATGGTGGGGTAACAGAA |
| 663 | 3\|sg_hRPL9_CC_2\|RPL9 | RPL9 | GAAAGGAACTGGCTACCGTT |
| 664 | 3\|sg_hRPL9_CC_3\|RPL9 | RPL9 | AGGGCTTCCGTTACAAGATG |
| 665 | 3\|sg_hRPL9_CC_4\|RPL9 | RPL9 | GAACAAGCAACACCTAAAAG |
| 666 | 3\|sg_hSF3A3_CC_1\|SF3A3 | SF3A3 | TGAGGAGAAGGAACGGCTCA |
| 667 | 3\|sg_hSF3A3_CC_2\|SF3A3 | SF3A3 | GGAAGAATGCAGAGTATAAG |
| 668 | 3\|sg_hSF3A3_CC_3\|SF3A3 | SF3A3 | GGAATTTGAGGAACTCCTGA |
| 669 | 3\|sg_hSF3A3_CC_4\|SF3A3 | SF3A3 | GCTCACCGGCCATCCAGGAA |
| 670 | 3\|sg_hSF3B3_CC_1\|SF3B3 | SF3B3 | ACTGGCCAGGAACGATGCGA |
| 671 | 3\|sg_hSF3B3_CC_2\|SF3B3 | SF3B3 | GCAGCTCCAAGATCTTCCCA |
| 672 | 3\|sg_hSF3B3_CC_3\|SF3B3 | SF3B3 | GAATGAGTACACAGAACGGA |
| 673 | 3\|sg_hSF3B3_CC_4\|SF3B3 | SF3B3 | GGAGCAGGACAAGGTCGGGG |

### Example 2—BRD9 Degrader Depletes BRD9 Protein

The following example demonstrates the depletion of the BRD9 protein in synovial sarcoma cells treated with a BRD9 degrader.

### Procedure:

Cells were treated with DMSO or the BRD9 degrader, Compound 1 (also known as dBRD9, see Remillard et al, Angew. Chem. Int. Ed. Engl. 56(21):5738-5743 (2017); see structure of Compound 1 below), for indicated doses and timepoints.

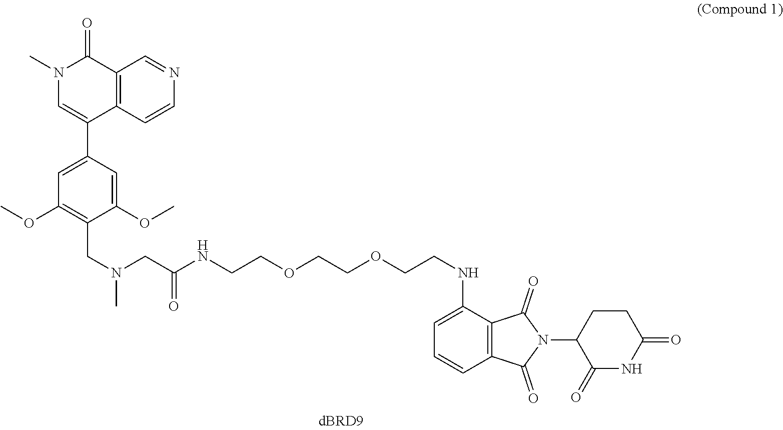 [SMILES: COc1cc(-c2cn(C)c(=O)c3cnccc23)cc(OC)c1CN(C)CC(=O)NCCOCCOCCNc1cccc2c1C(=O)N(C1CCC(=O)NC1=O)C2=O]

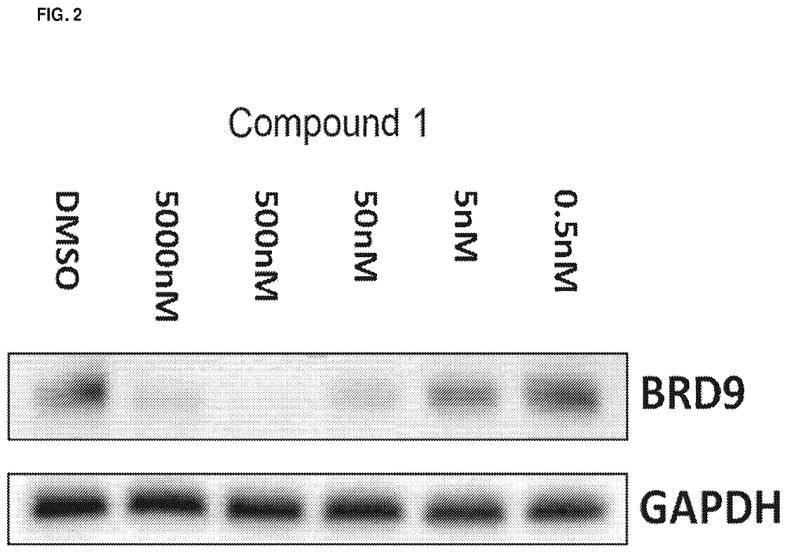

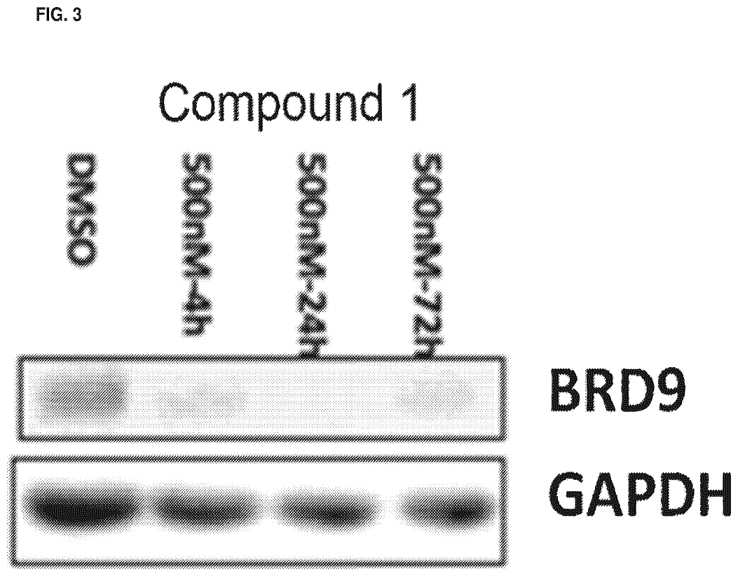

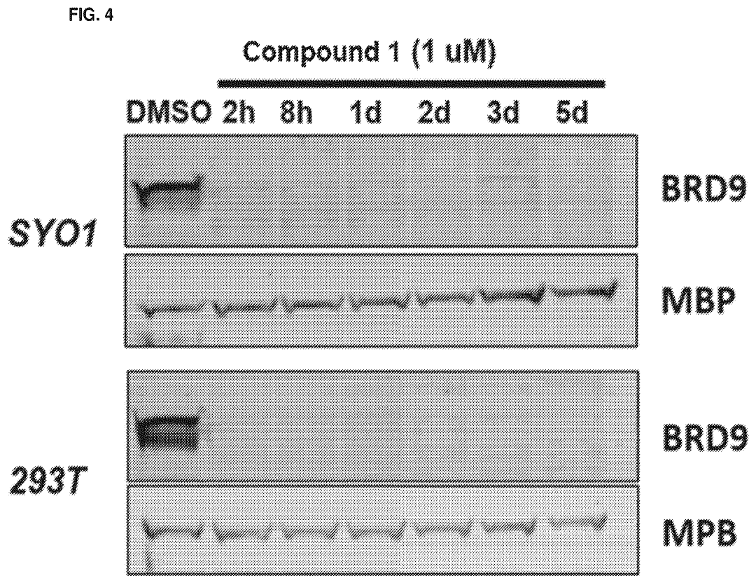

Whole cell extracts were fractionated by SDS-PAGE and transferred to a polyvinylidene difluoride membrane using a transfer apparatus according to the manufacturer's protocols (Bio-Rad). After incubation with 5% nonfat milk in TBST (10 mM Tris, pH 8.0, 150 mM NaCl, 0.5% Tween 20) for 60 minutes, the membrane was incubated with antibodies against BRD9 (1:1,000, Bethyl laboratory A303-781A), GAPDH (1:5,000, Cell Signaling Technology), and/or MBP (1:1,000, BioRad) overnight at 4° C. Membranes were washed three times for 10 min and incubated with anti-mouse or anti-rabbit antibodies conjugated with either horseradish peroxidase (HRP, FIGS. 2-3) or IRDye (FIG. 4, 1:20,000, LI-COR) for at least 1 h. Blots were washed with TBST three times and developed with either the ECL system according to the manufacturer's protocols (FIGS. 2-3) or scanned on an Odyssey CLx Imaging system (FIG. 4).

### Results:

Treatment of SYO1 synovial sarcoma cells with the BRD9 degrader Compound 1 results in dose dependent (FIG. 2) and time dependent (FIG. 3) depletion of BRD9 in the cells. Further, as shown in FIG. 4, the depletion of BRD9 by Compound 1 is replicated in a non-synovial sarcoma cell line (293T) and may be sustained for at least 5 days.

### Example 3—Inhibition of Growth of Synovial Cell Lines by BRD9 Inhibitors and BRD9 Degraders

The following example demonstrates that BRD9 degraders and inhibitors selectively inhibit growth of synovial sarcoma cells.

### Procedures:

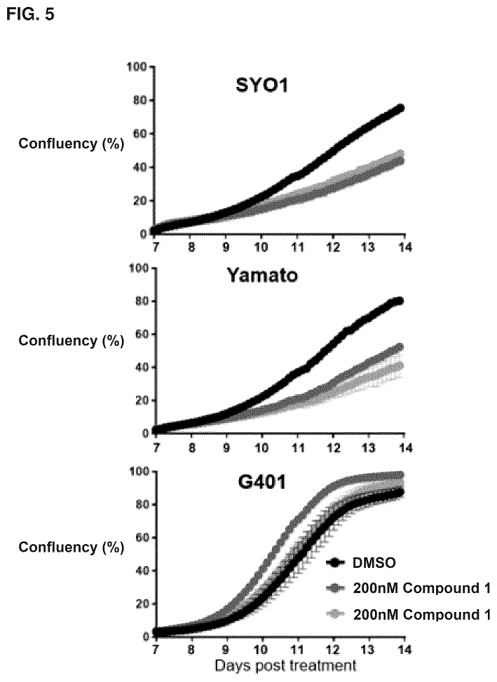

Cells were treated with DMSO or the BRD9 degrader, Compound 1, at indicated concentrations, and proliferation was monitored from day 7 to day 14 by measuring confluency overtime using an IncuCyte live cell analysis system (FIG. 5). Growth medium and compounds were refreshed every 3-4 days.

Cells were seeded into 12-well plates and treated with DMSO, 1 μM BRD9 inhibitor, Compound 2 (also known as BI-7273, see Martin et al, J Med Chem. 59(10):4462-4475 (2016); see structure of Compound 2 below), or 1 μM BRD9 degrader, Compound 1.

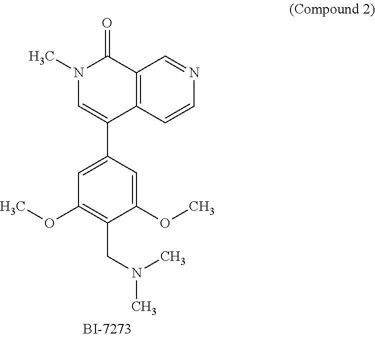

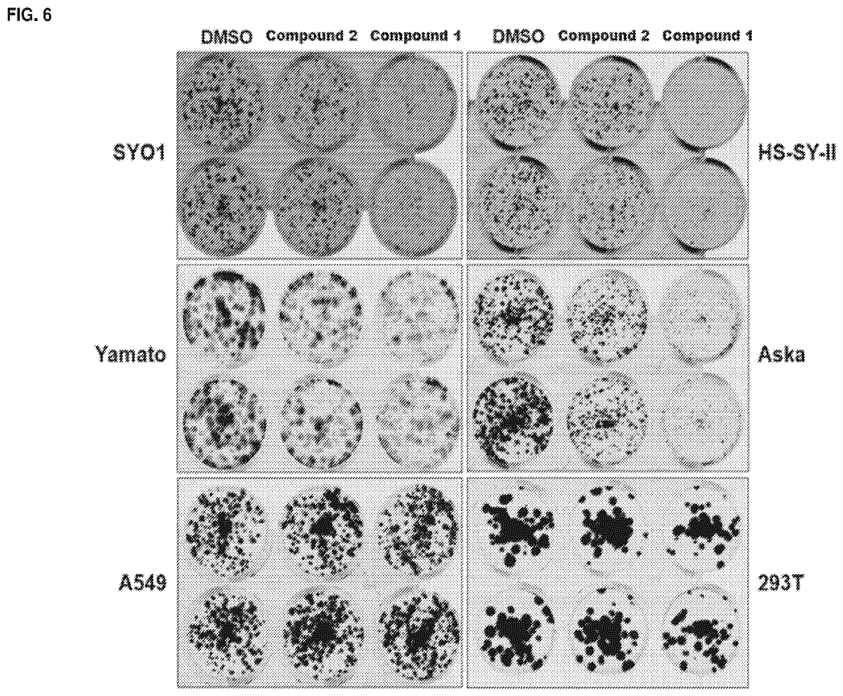

The number of cells was optimized for each cell line. Growth medium and compounds were refreshed every 3-5 days. SYO1, Yamato, A549, 293T and HS-SY-II cells were fixed and stained at day 11. ASKA cells were fixed and stained at day 23. Staining was done by incubation with crystal violet solution (0.5 g Crystal Violet, 27 ml 37% Formaldehyde, 100 mL 10×PBS, 10 mL Methanol, 863 dH20 to 1 L) for 30 min followed by 3× washes with water and drying the plates for at least 24h at room temperature. Subsequently plates were scanned on an Odyssey CLx Imaging system (FIG. 6).

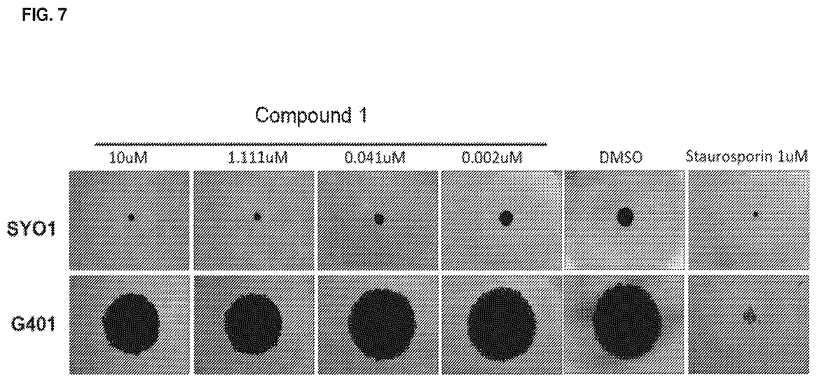

Cells were seeded into 96-well ultra low cluster plate (Costar, #7007) in 200 μL complete media and treated at day 2 with DMSO, Staurosporin, or BRD9 degarder, Compound 1, at indicated doses (FIG. 7). Media and compounds were changed every 5 d and cell colonies were imaged at day 14.

### Results:

As shown in FIGS. 5, 6, and 7, treatment of synovial sarcoma cell lines (SYO1, Yamato, HS-SY-II, and ASKA) with a BRD9 inhibitor, Compound 2, or a BRD9 degrader, Compound 1, results in inhibition of the growth of the cells, but does not result in inhibition of the growth of non-synovial control cancer cell lines (293T, A549, G401).

### Example 4—Selective Inhibition of Growth of Synovial Cell Lines by BRD9 Degraders and BRD9 Binders

The following example demonstrates that BRD9 degraders and binders selectively inhibit growth of synovial sarcoma cells.

### Procedure:

Cells were seeded into 6-well or 12-well plates and were treated daily with a BRD9 degrader (Compound 1), a bromo-domain BRD9 binder (Compound 2), E3 ligase binder (lenalidomide), DMSO, or staurosporin (positive control for cell killing), at indicated concentrations. The number of cells was optimized for each cell line. Growth media was refreshed every 5 days. By day 14, medium was removed, cells were washed with PBS, and stained using 500 μL of 0.005% (w/v) crystal violet solution in 25% (v/v) methanol for at least 1 hour at room temperature. Subsequently plates were scanned on an Odyssey CLx Imaging system.

### Results:

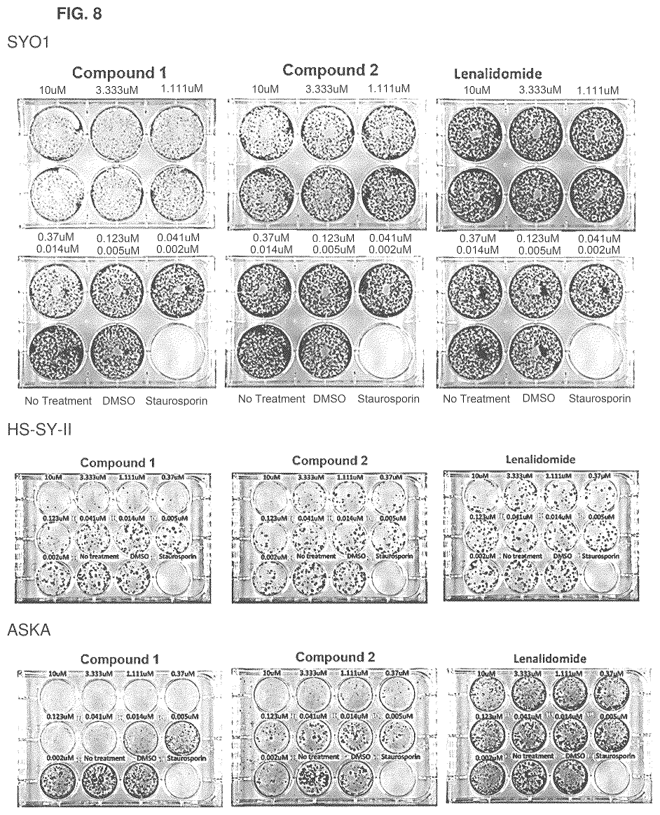

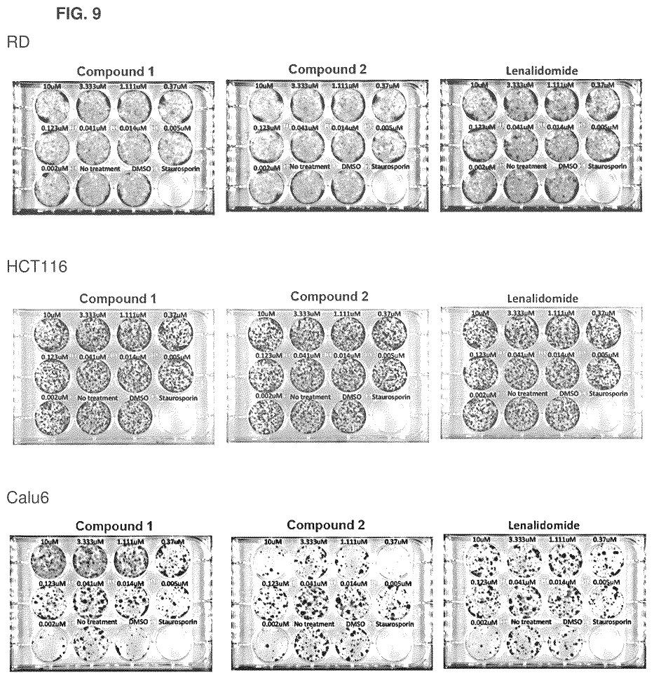

As shown in FIGS. 8 and 9, treatment of synovial sarcoma cell lines (SYO1, HS-SY-II, and ASKA) with Compound 1 or Compound 2 resulted in inhibition of the growth of the cells, but did not result in inhibition of the growth of non-synovial control cancer cell lines (RD, HCT116, and Calu6). Overall, Compound 1 showed most significant growth inhibition in all synovial cell lines.

### Example 5—Inhibition of Cell Growth in Synovial Sarcoma Cells

The following example shows that BRD9 degraders inhibit cell growth and induce apoptosis in synovial sarcoma cells.

### Procedure:

SYO1 cells were treated for 8 or 13 days with DMSO, a BRD9 degrader (Compound 1) at 200 nM or 1 μM, or an E3 ligase binder (lenalidomide) at 200 nM. Compounds were refreshed every 5 days. Cell cycle analysis was performed using the Click-iT™ Plus EdU Flow Cytometry Assay (Invitrogen). The apoptosis assay was performed using the Annexin V-FITC Apoptosis Detection Kit (Sigma A9210). Assays were performed according to the manufacturer's protocol.

### Results:

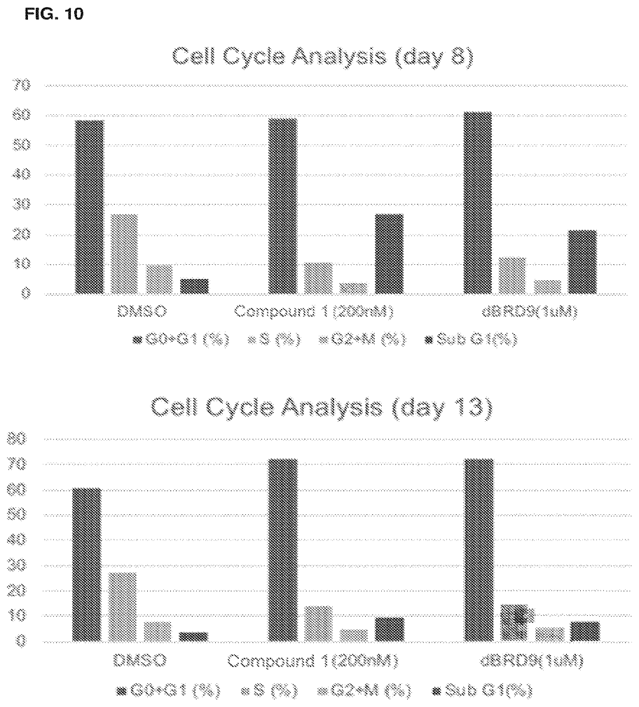

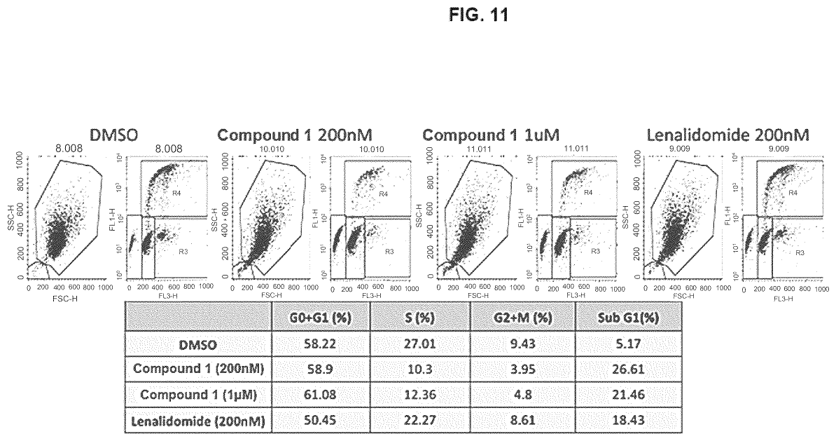

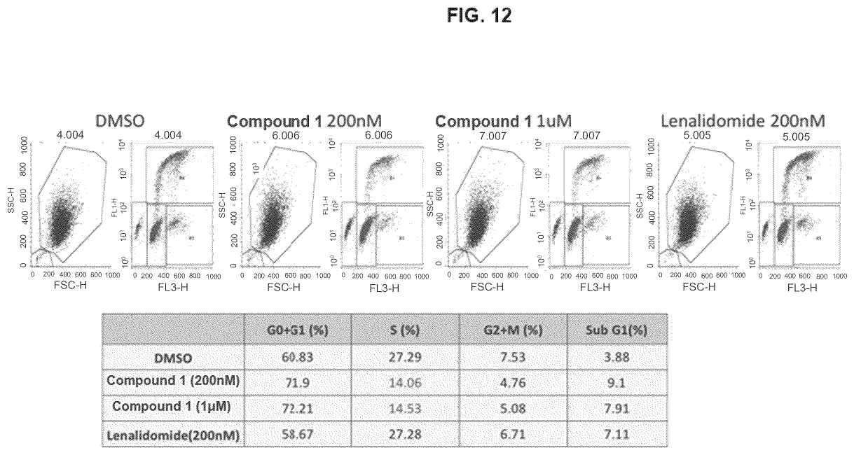

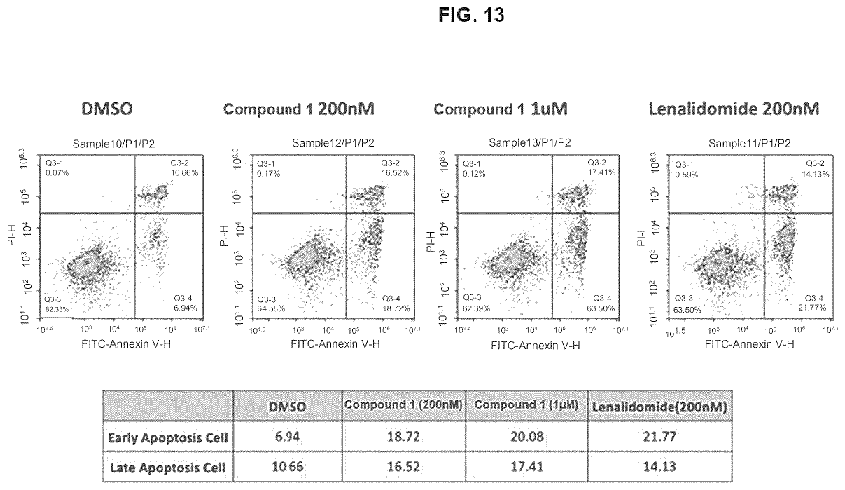

As shown in FIGS. 10-13, treatment with Compound 1 for 8 or 13 days resulted in reduced numbers of cells in the S-phase of the cell cycle as compared to DMSO and lenalidomide. Treatment with Compound 1 for 8 days also resulted in increased numbers of early- and late-apoptotic cells as compared to DMSO controls.

### Example 6—Composition for SS18-SSX1-BAF

The following example shows the identification of BRD9 as a component of SS18-SSX containing BAF complexes.

### Procedure:

A stable 293T cell line expressing HA-SS18SSX1 was generated using lentiviral integration. SS18-SSX1 containing BAF complexes were subject to affinity purification and subsequent mass spectrometry analysis revealed SS18-SSX1 interacting proteins.

### Results:

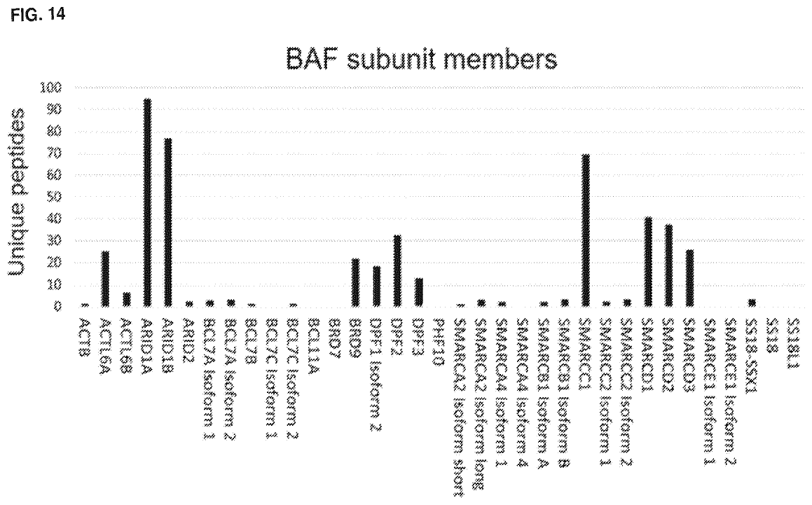

As shown in FIG. 14, BAF complexes including the SS18-SSX fusion protein also included BRD9. More than 5 unique peptides were identified for ARID1A (95 peptides), ARID1B (77 peptides), SMARCC1 (69 peptides), SMARCD1 (41 peptides), SMARCD2 (37 peptides), DPF2 (32 peptides), SMARCD3 (26 peptides), ACTL6A (25 peptides), BRD9 (22 peptides), DPF1 Isoform 2 (18 peptides), DPF3 (13 peptides), and ACTL6B (6 peptides).

### Example 7—Preparation of 1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)azetidine-3-carboxamide formic acid (compound D1 formic acid)

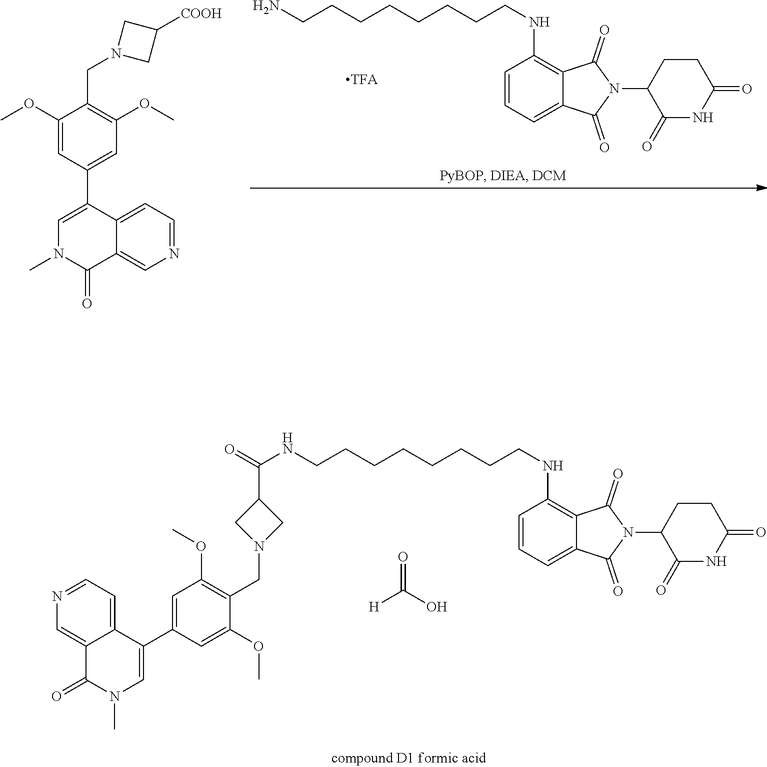 [SMILES: COc1cc(-c2cn(C)c(=O)c3cnccc23)cc(OC)c1CN1CC(C(=O)O)C1 ; NCCCCCCCCNc1cccc2c1C(=O)N(C1CCC(=O)NC1=O)C2=O ; COc1cc(-c2cn(C)c(=O)c3cnccc23)cc(OC)c1CN1CC(C(=O)NCCCCCCCCNc2cccc3c2C(=O)N(C2CCC(=O)NC2=O)C3=O)C1 ; O=CO]

To a stirred mixture of 4-[(8-aminooctyl)amino]-2-(2,6-dioxopiperidin-3-yl)-2,3-dihydro-1H-isoindole-1,3-dione trifluoroacetic acid salt (50 mg, 0.097 mmol, 1 equiv) and 1-[[2,6-dimethoxy-4-(2-methyl-1-oxo-1,2-dihydro-2,7-naphthyridin-4-yl)phenyl]methyl]azetidine-3-carboxylic acid trifluoroacetic acid salt (50.87 mg, 0.097 mmol, 1 equiv) in DCM (2 mL, 31.460 mmol, 323.73 equiv) was added DIEA (37.68 mg, 0.292 mmol, 3 equiv) and PyBOP (75.86 mg, 0.146 mmol, 1.5 equiv). The mixture was stirred for 2 hours at room temperature, and then it was concentrated under vacuum. The residue was purified by Prep-HPLC (conditions: X Select CSH Prep C18 OBD Column, 5 μm, 19*150 mm; mobile phase, Water (0.1% FA) and ACN (25% Phase B up to 45% in 8 minutes); Detector, UV). This resulted in 1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)azetidine-3-carboxamide formic acid (4 mg, 4.81%) as a yellow solid. ¹H NMR (400 MHz, Methanol-d4) δ 9.54 (s, 1H), 8.69 (d, J=5.7 Hz, 1H), 8.54 (s, 1H), 7.76 (s, 1H), 7.62 (d, J=5.8 Hz, 1H), 7.60-7.51 (m, 1H), 7.04 (d, J=7.8 Hz, 2H), 6.83 (s, 2H), 5.07 (dd, J=12.5, 5.5 Hz, 1H), 4.31 (s, 2H), 4.05 (s, 4H), 3.94 (s, 6H), 3.71 (s, 3H), 3.52-3.45 (s, 2H), 3.22 (t, J=7.0 Hz, 2H), 2.91-2.66 (m, 4H), 2.14-2.11 (m, 1H), 1.67 (q, J=7.3 Hz, 2H), 1.54 (d, J=7.3 Hz, 2H), 1.45-1.38 (m, 8H). LCMS (ESI) m/z: [M+H]⁺=792.36.

### Example 8—Preparation of 4-(2-[1-[2-([[2,6-dimethoxy-4-(2-methyl-1-oxo-1,2-dihydro-2,7-naphthyridin-4-yl)phenyl]methyl](methyl)amino)acetyl]-[4,4-bipiperidin]-1-yl]-2-oxoethoxy)-2-(2,6-dioxopiperidin-3-yl)-2,3-dihydro-1H-isoindole-1,3-dione (compound D2)

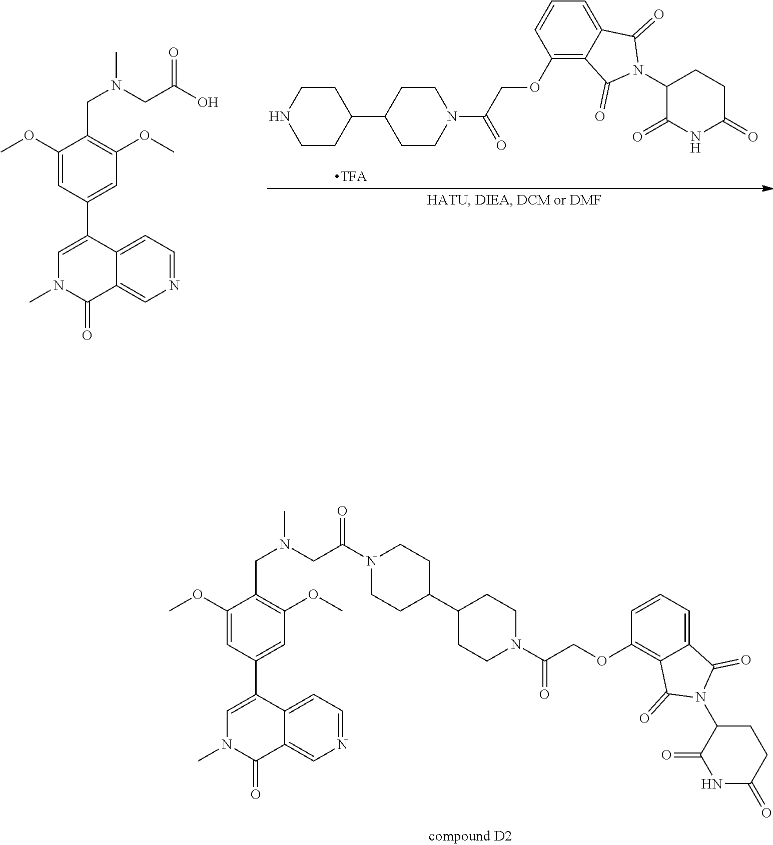 [SMILES: COc1cc(-c2cn(C)c(=O)c3cnccc23)cc(OC)c1CN(C)CC(=O)O ; O=C1CCC(N2C(=O)c3cccc(OCC(=O)N4CCC(C5CCNCC5)CC4)c3C2=O)C(=O)N1 ; COc1cc(-c2cn(C)c(=O)c3cnccc23)cc(OC)c1CN(C)CC(=O)N1CCC(C2CCN(C(=O)COc3cccc4c3C(=O)N(C3CCC(=O)NC3=O)C4=O)CC2)CC1]

To a stirred solution of 2-([[2,6-dimethoxy-4-(2-methyl-1-oxo-1,2-dihydro-2,7-naphthyridin-4-yl)phenyl]methyl](methyl)amino)acetic acid (19.99 mg, 0.050 mmol, 1 equiv) and DIPEA (19.50 mg, 0.151 mmol, 3 equiv) in DMF (3 mL) was added PyBOP (28.68 mg, 0.075 mmol, 1.5 equiv) and 4-(2-[[4,4-bipiperidin]-1-yl]-2-oxoethoxy)-2-(2,6-dioxopiperidin-3-yl)-2,3-dihydro-1H-isoindole-1,3-dione trifluoroacetic acid salt (30 mg, 0.050 mmol, 1 equiv). The solution was stirred for 2 hours at room temperature. The resulting mixture was purified by Prep-HPLC (conditions: XSelect CSH Prep C18 OBD Column, 5 μm, 19*150 mm; Mobile Phase A: Water (0.1% FA), Mobile Phase B: ACN; Flow rate: 25 mL/minute; Gradient: 5% B to 30% B in 8 minutes; 254 nm; Rt: 7.56 minutes) to afford 4-(2-[1-[2-([[2,6-dimethoxy-4-(2-methyl-1-oxo-1,2-dihydro-2,7-naphthyridin-4-yl)phenyl]methyl](methyl)amino)acetyl]-[4,4-bipiperidin]-1-yl]-2-oxoethoxy)-2-(2,6-dioxopiperidin-3-yl)-2,3-dihydro-1H-isoindole-1,3-dione (19 mg, 43.83%) as a white solid. ¹H NMR (300 MHz, Methanol-d4) δ 9.53 (d, J=0.8 Hz, 1H), 8.69 (d, J=5.8 Hz, 1H), 8.56 (s, 0.3H), 7.76 (s, 2H), 7.64 (d, J=5.7 Hz, 1H), 7.49 (d, J=7.3 Hz, 1H), 7.36 (d, J=8.2 Hz, 1H), 6.80 (s, 2H), 5.14 (t, J=15.7 Hz, 3H), 4.60-4.43 (m, 3H), 4.02 (d, J=13.6 Hz, 4H), 3.91 (s, 6H), 3.71 (s, 3H), 3.58 (s, 2H), 3.15-2.59 (m, 6H), 2.53 (s, 3H), 2.15 (s, 1H), 1.85-1.67 (m, 4H), 1.41-1.16 (m, 6H). LCMS (ESI) m/z: [M+H]+=862.

### Example 9—Preparation of 1-[[2,6-dimethoxy-4-(2-methyl-1-oxo-1,2-dihydro-2,7-naphthyridin-4-yl)phenyl]methyl]-N-(5-[[2-(2,6-dioxopiperidin-3-yl)-1,3-dioxo-2,3-dihydro-1H-isoindol-4-yl]amino]pentyl) azetidine-3-carboxamide (compound D3)

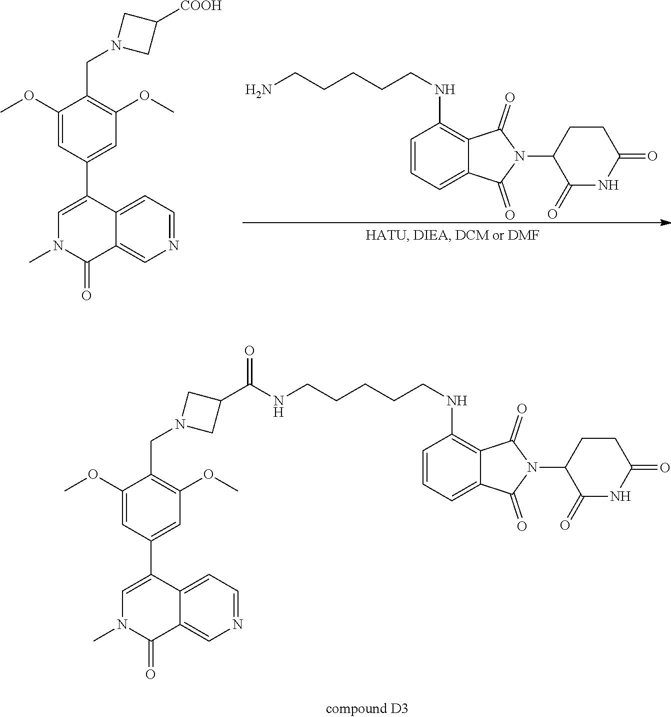 [SMILES: COc1cc(-c2cn(C)c(=O)c3cnccc23)cc(OC)c1CN1CC(C(=O)O)C1 ; NCCCCCNc1cccc2c1C(=O)N(C1CCC(=O)NC1=O)C2=O ; COc1cc(-c2cn(C)c(=O)c3cnccc23)cc(OC)c1CN1CC(C(=O)NCCCCCNc2cccc3c2C(=O)N(C2CCC(=O)NC2=O)C3=O)C1]

To a stirred mixture of 1-[[2,6-dimethoxy-4-(2-methyl-1-oxo-1,2-dihydro-2,7-naphthyridin-4-yl)phenyl]methyl]azetidine-3-carboxylic acid trifluoroacetic acid salt(55.40 mg, 0.106 mmol, 1 equiv) and 4-[(5-aminopentyl)amino]-2-(2,6-dioxopiperidin-3-yl)-2,3-dihydro-1H-isoindole-1,3-dione; trifluoroacetic acid salt (50 mg, 0.106 mmol, 1 equiv) in DCM (2 mL) was added DIEA (41.04 mg, 0.318 mmol, 3 equiv) and PyBOP (82.62 mg, 0.159 mmol, 1.5 equiv). The mixture was stirred for 2 hours at room temperature, and then it was concentrated under vacuum. The residue was purified by Prep-HPLC (conditions: X Select CSH Prep C18 OBD Column, 5 μm, 19*150 mm; mobile phase, Water (0.1% FA) and ACN (15% Phase B up to 35% in 8 minutes); Detector, UV). This resulted in 6 mg (6.98%) of 1-[[2,6-dimethoxy-4-(2-methyl-1-oxo-1,2-dihydro-2,7-naphthyridin-4-yl)phenyl]methyl]-N-(5-[[2-(2,6-dioxopiperidin-3-yl)-1,3-dioxo-2,3-dihydro-1H-isoindol-4-yl]amino]pentyl) azetidine-3-carboxamide formate as a yellow solid. ¹H NMR (400 MHz, Methanol-d4) δ 9.54 (s, 1H), 8.69 (d, J=5.8 Hz, 1H), 8.53 (s, 1H), 7.76 (s, 1H), 7.65-7.51 (m, 2H), 7.05 (dd, J=7.8, 6.0 Hz, 2H), 6.83 (s, 2H), 5.11-5.02 (m, 1H), 4.57 (s, 1H), 4.36 (s, 2H), 4.10 (s, 4H), 3.95 (s, 6H), 3.71 (s, 3H), 3.36-3.26 (m, 3H), 2.91-2.68 (m, 3H), 2.12 (d, J=10.0 Hz, 1H), 1.76-1.67 (m, 2H), 1.60 (q, J=7.3, 6.8 Hz, 2H), 1.49 (d, J=7.1 Hz, 2H). LCMS (ESI) m/z: [M+H]⁺=750.32.

### Example 10—Preparation of N-[8-[(1-[[2,6-dimethoxy-4-(2-methyl-1-oxo-1,2-dihydro-2,7-naphthyridin-4-yl)phenyl]methyl]azetidin-3-yl)formamido]octyl]-2-[[2-(2,6-dioxopiperidin-3-yl)-1,3-dioxo-2,3-dihydro-1H-isoindol-4-yl]oxy]acetamide formic acid (compound D4 formic acid)

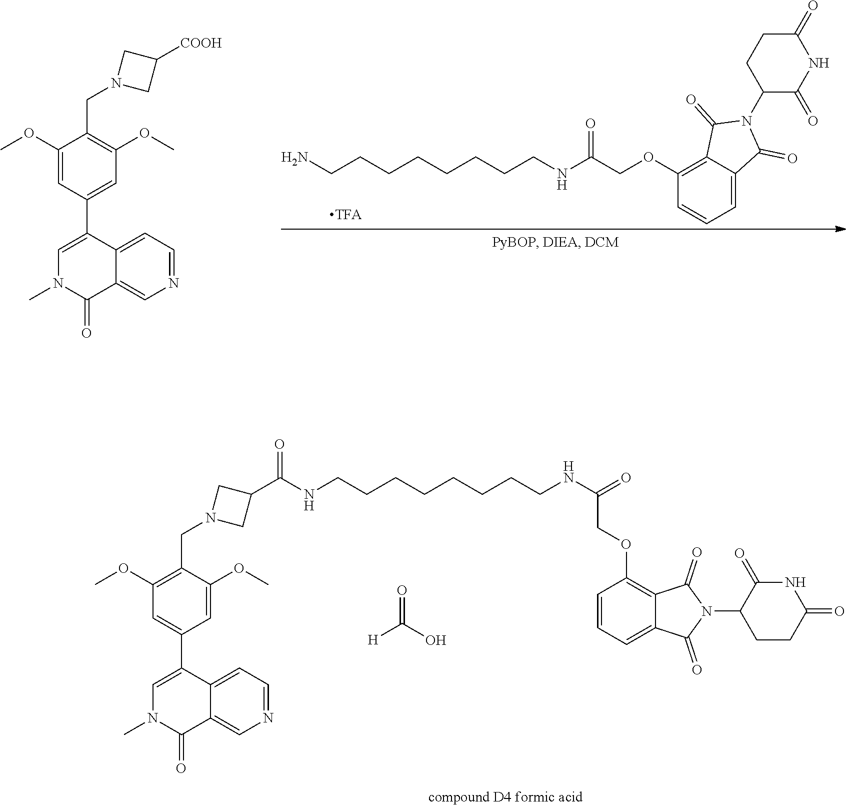 [SMILES: COc1cc(-c2cn(C)c(=O)c3cnccc23)cc(OC)c1CN1CC(C(=O)O)C1 ; NCCCCCCCCNC(=O)COc1cccc2c1C(=O)N(C1CCC(=O)NC1=O)C2=O ; COc1cc(-c2cn(C)c(=O)c3cnccc23)cc(OC)c1CN1CC(C(=O)NCCCCCCCCNC(=O)COc2cccc3c2C(=O)N(C2CCC(=O)NC2=O)C3=O)C1 ; O=CO]

To a stirred mixture of 1-[[2,6-dimethoxy-4-(2-methyl-1-oxo-2,7-naphthyridin-4-yl)phenyl]methyl]azetidine-3-carboxylic acid; trifluoroacetic acid salt (68.57 mg, 0.131 mmol, 1.50 equiv) and N-(8-aminooctyl)-2-[[2-(2,6-dioxopiperidin-3-yl)-1,3-dioxoisoindol-4-yl]oxy]acetamide trifluoroacetic acid salt (50.00 mg, 0.087 mmol, 1.00 equiv) in DCM (2.00 mL) was added DIEA (67.72 mg, 0.524 mmol, 6.00 equiv) and PyBOP (68.17 mg, 0.131 mmol, 1.50 equiv). The mixture was stirred for 2 hours at room temperature, and then it was concentrated under vacuum. The residue was purified by Prep-HPLC (conditions: X Bridge Shield RP18 OBD Column, 5 μm, 19*150 mm; mobile phase, Water (0.1% FA) and ACN (20% Phase B up to 32% in 7 minutes); Detector, UV). This resulted in N-[8-[(1-[[2,6-dimethoxy-4-(2-methyl-1-oxo-1,2-dihydro-2,7-naphthyridin-4-yl)phenyl]methyl]azetidin-3-yl)formamido]octyl]-2-[[2-(2,6-dioxopiperidin-3-yl)-1,3-dioxo-2,3-dihydro-1H-isoindol-4-yl]oxy]acetamide formic acid (12 mg, 14.77%) as a white solid. ¹H NMR (400 MHz, Methanol-d4) δ 9.53 (s, 1H), 8.68 (d, J=5.8 Hz, 1H), 7.87-7.78 (m, 1H), 7.75 (s, 1H), 7.63 (d, J=5.8 Hz, 1H), 7.55 (d, J=7.4 Hz, 1H), 7.44 (d, J=8.4 Hz, 1H), 6.80 (s, 2H), 5.15 (dd, J=12.6, 5.3 Hz, 1H), 4.76 (s, 2H), 4.14 (s, 2H), 3.92 (s, 6H), 3.80 (s, 4H), 3.71 (s, 3H), 3.20 (t, J=7.0 Hz, 2H), 2.94-2.71 (m, 6H), 2.15 (s, 1H), 1.58 (d, J=7.9 Hz, 2H), 1.51 (s, 2H), 1.35 (s, 8H). LCMS (ESI) m/z: [M+H]⁺=850.37.

### Example 11—Preparation of N-(1-[[2,6-dimethoxy-4-(2-methyl-1-oxo-1,2-dihydro-2,7-naphthyridin-4-yl)phenyl]methyl]azetidin-3-yl)-6-[[2-(2,6-dioxopiperidin-3-yl)-1,3-dioxo-2,3-dihydro-1H-isoindol-4-yl]oxy]hexanamide (compound D5)

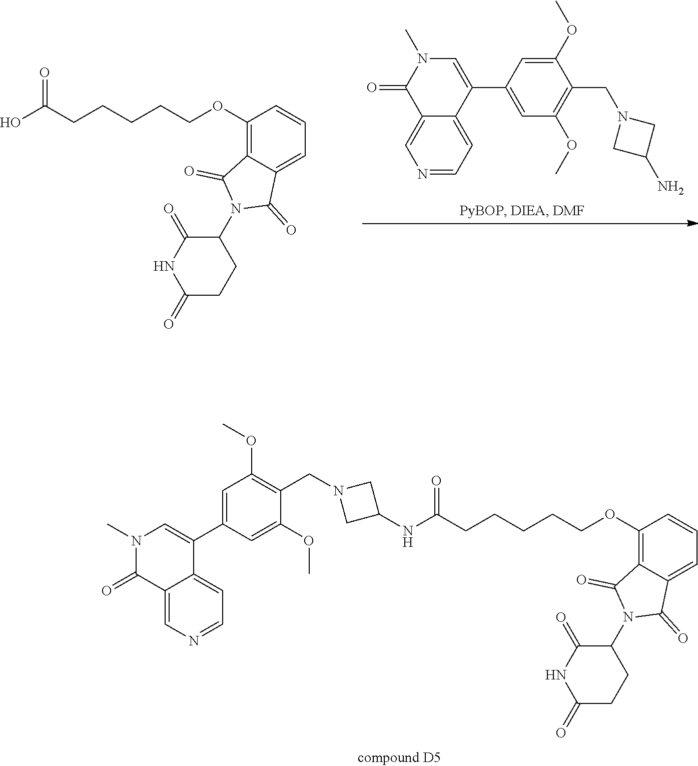 [SMILES: O=C(O)CCCCCOc1cccc2c1C(=O)N(C1CCC(=O)NC1=O)C2=O ; COc1cc(-c2cn(C)c(=O)c3cnccc23)cc(OC)c1CN1CC(N)C1 ; COc1cc(-c2cn(C)c(=O)c3cnccc23)cc(OC)c1CN1CC(NC(=O)CCCCCOc2cccc3c2C(=O)N(C2CCC(=O)NC2=O)C3=O)C1]

To a solution of 6-[[2-(2,6-dioxopiperidin-3-yl)-1,3-dioxo-2,3-dihydro-1H-isoindol-4-yl]oxy]hexanoic acid (50.00 mg, 0.129 mmol, 1.00 eq.) and DIEA (49.92 mg, 0.386 mmol, 3 eq.) in DCM (2.00 mL, 31.460 mmol, 244.37 eq.) was added PyBOP (100.49 mg, 0.193 mmol, 1.5 eq.) and 4-[4-[(3-aminoazetidin-1-yl)methyl]-3,5-dimethoxyphenyl]-2-methyl-1,2-dihydro-2,7-naphthyridin-1-one (48.98 mg, 0.129 mmol, 1 eq.). The resulting solution was stirred at room temperature for 1 hour. The crude product (50 mg) was purified by Prep-HPLC (conditions: XSelect CSH Prep C18 OBD Column, 5 μm, 19*150 mm; Mobile Phase A: Water (0.1% FA), Mobile Phase B: ACN; Flow rate: 25 mL/minute; Gradient: 10% B to 30% B in 8 minutes; 254 nm; Rt: 6.57 minutes) to afford N-(1-[[2,6-dimethoxy-4-(2-methyl-1-oxo-1,2-dihydro-2,7-naphthyridin-4-yl)phenyl]methyl]azetidin-3-yl)-6-[[2-(2,6-dioxopiperidin-3-yl)-1,3-dioxo-2,3-dihydro-1H-isoindol-4-yl]oxy]hexanamide (14.8 mg, 15.31%) as a white solid. ¹H NMR (400 MHz, Methanol-d4) δ 9.54 (s, 1H), 8.69 (d, J=5.7 Hz, 1H), 7.82-7.73 (m, 2H), 7.65-7.58 (m, 1H), 7.44 (dd, J=7.9, 3.2 Hz, 2H), 6.83 (s, 2H), 5.10 (dd, J=12.4, 5.4 Hz, 1H), 4.60-4.47 (m, 1H), 4.34 (s, 2H), 4.25 (t, J=6.1 Hz, 2H), 4.18 (s, 2H), 3.94 (s, 8H), 3.71 (s, 3H), 2.87-2.64 (m, 3H), 2.30 (t, J=7.3 Hz, 2H), 2.17-2.09 (m, 1H), 1.90 (p, J=6.4 Hz, 2H), 1.75 (p, J=7.4 Hz, 2H), 1.61 (q, J=8.0 Hz, 2H). LCMS (ESI) m/z: [M+H]+=751.25.

### Example 12—Preparation of 4-[2-[1-(1-[[2,6-dimethoxy-4-(2-methyl-1-oxo-1,2-dihydro-2,7-naphthyridin-4-yl)phenyl]methyl]azetidine-3-carbonyl)-[4,4-bipiperidin]-1-yl]-2-oxoethoxy]-2-(2,6-dioxo piperidin-3-yl)-2,3-dihydro-1H-isoindole-1,3-dione formic acid (compound D6 formic acid)

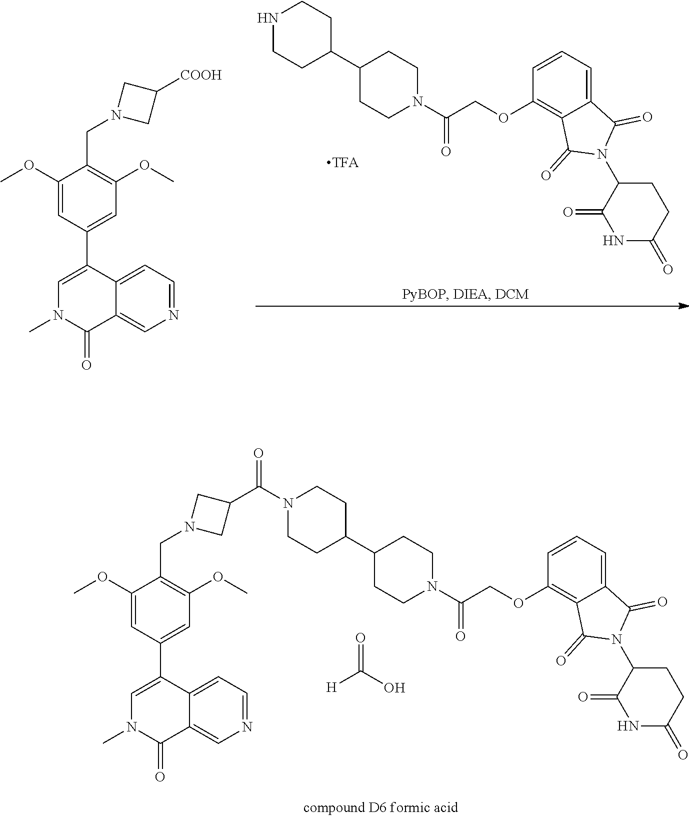 [SMILES: COc1cc(-c2cn(C)c(=O)c3cnccc23)cc(OC)c1CN1CC(C(=O)O)C1 ; O=C1CCC(N2C(=O)c3cccc(OCC(=O)N4CCC(C5CCNCC5)CC4)c3C2=O)C(=O)N1 ; COc1cc(-c2cn(C)c(=O)c3cnccc23)cc(OC)c1CN1CC(C(=O)N2CCC(C3CCN(C(=O)COc4cccc5c4C(=O)N(C4CCC(=O)NC4=O)C5=O)CC3)CC2)C1 ; O=CO]

To a stirred mixture of 1-[[2,6-dimethoxy-4-(2-methyl-1-oxo-2,7-naphthyridin-4-yl)phenyl]methyl]azetidine-3-carboxylic acid trifluoroacetic acid salt (26.32 mg, 0.050 mmol, 1.50 equiv) and 4-(2-[[4,4-bipiperidin]-1-yl]-2-oxoethoxy)-2-(2,6-dioxopiperidin-3-yl)isoindole-1,3-dione trifluoroacetic acid salt (20.00 mg, 0.034 mmol, 1.00 equiv) in DCM (2 mL) was added DIEA (26.00 mg, 0.201 mmol, 6.00 equiv) and PyBOP (26.17 mg, 0.050 mmol, 1.50 equiv). The mixture was stirred for 2 hours at room temperature, and then it was concentrated under vacuum. The residue was purified was purified by Prep-HPLC (conditions: X Select CSH Prep C18 OBD Column, 5 μm, 19*150 mm; mobile phase, Water (0.1% FA) and ACN (8% Phase B up to 22% in 8 minutes); Detector, UV). This resulted in 4-[2-[1-(1-[[2,6-dimethoxy-4-(2-methyl-1-oxo-1,2-dihydro-2,7-naphthyridin-4-yl)phenyl]methyl]azetidine-3-carbonyl)-[4,4-bipiperidin]-1-yl]-2-oxoethoxy]-2-(2,6-dioxo piperidin-3-yl)-2,3-dihydro-1H-isoindole-1,3-dione formic acid (3.5 mg, 10.89%) as a white solid. ¹H NMR (300 MHz, Methanol-d4) δ 9.54 (d, J=0.8 Hz, 1H), 8.69 (d, J=5.7 Hz, 1H), 8.56 (s, 1H), 7.84-7.72 (m, 2H), 7.63 (d, J=5.8 Hz, 1H), 7.51 (d, J=7.3 Hz, 1H), 7.38 (d, J=8.6 Hz, 1H), 6.81 (s, 2H), 5.31-4.98 (m, 3H), 4.68-4.44 (m, 2H), 4.16 (s, 2H), 3.93 (s, 10H), 3.79-3.56 (m, 5H), 3.09-2.93 (m, 2H), 2.93-2.61 (m, 6H), 2.15 (d, J=10.4 Hz, 1H), 1.86-1.67 (m, 4H), 1.50-1.25 (m, 3H), 1.23-1.04 (m, 2H). LCMS (ESI) m/z: [M+H]⁺=874.37.

### Example 13—Preparation of 1-[[2,6-dimethoxy-4-(2-methyl-1-oxo-1,2-dihydro-2,7-naphthyridin-4-yl)phenyl]methyl]-N-[2-[2-(2-[[2-(2,6-dioxopiperidin-3-yl)-1,3-dioxo-2,3-dihydro-1H-isoindol-4-yl]amino]ethoxy)ethoxy]ethyl]azetidine-3-carboxamide formic acid (compound D7 formic acid)

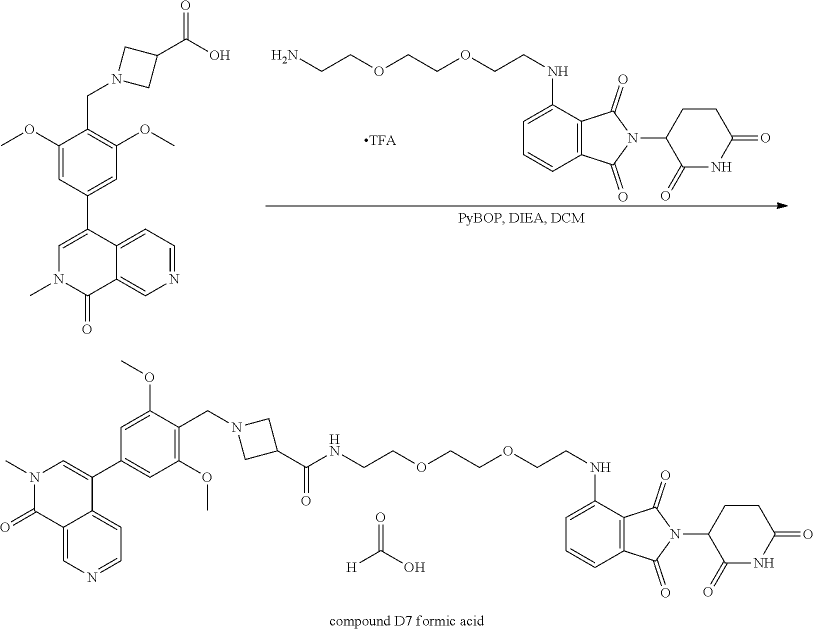

To a stirred mixture of 1-[[2,6-dimethoxy-4-(2-methyl-1-oxo-1,2-dihydro-2,7-naphthyridin-4-yl)phenyl]methyl]azetidine-3-carboxylic acid trifluoroacetic acid salt (75.73 mg, 0.145 mmol, 1.5 equiv) and 4-([2-[2-(2-aminoethoxy)ethoxy]ethyl]amino)-2-(2,6-dioxopiperidin-3-yl)-2,3-dihydro-1H-isoindole-1,3-dione trifluoroacetic acid salt (50 mg, 0.096 mmol, 1 equiv) in DCM (2 mL) was added DIEA (74.79 mg, 0.579 mmol, 6 equiv) and PyBOP (75.28 mg, 0.145 mmol, 1.5 equiv). The mixture was stirred for 2 hours at room temperature, and then it was concentrated under vacuum. The residue was purified by Prep-HPLC (conditions: X Select CSH Prep C18 OBD Column, 5 μm, 19*150 mm; mobile phase, Water (0.1% FA) and ACN (10% Phase B up to 32% in 8 minutes); Detector, UV). This resulted in 1-[[2,6-dimethoxy-4-(2-methyl-1-oxo-1,2-dihydro-2,7-naphthyridin-4-yl)phenyl]methyl]-N-[2-[2-(2-[[2-(2,6-dioxopiperidin-3-yl)-1,3-dioxo-2,3-dihydro-1H-isoindol-4-yl]amino]ethoxy)ethoxy]ethyl]azetidine-3-carboxamide formic acid (13.2 mg, 15.77%) as a yellow solid. ¹H NMR (400 MHz, Methanol-d4) δ 9.53 (s, 1H), 8.68 (d, J=5.8 Hz, 1H), 8.56 (s, 1H), 7.75 (s, 1H), 7.62 (d, J=5.9 Hz, 1H), 7.55 (dd, J=8.6, 7.1 Hz, 1H), 7.07 (dd, J=11.7, 7.8 Hz, 2H), 6.80 (s, 2H), 5.07 (dd, J=12.4, 5.5 Hz, 1H), 4.20 (s, 2H), 3.92 (s, 10H), 3.78-3.57 (m, 9H), 3.61-3.43 (m, 4H), 3.41 (td, J=5.2, 1.6 Hz, 2H), 2.88 (ddd, J=19.0, 14.0, 5.0 Hz, 1H), 2.80-2.64 (m, 3H). 2.17-2.08 (m, 1H). LCMS (ESI) m/z: [M+H]⁺=796.25.

### Example 14—Preparation of 1-[[2,6-dimethoxy-4-(2-methyl-1-oxo-1,2-dihydro-2,7-naphthyridin-4-yl)phenyl]methyl]-N-(8-[[2-(1-methyl-2,6-dioxopiperidin-3-yl)-1,3-dioxo-2,3-dihydro-1H-isoindol-4-yl]amino]octyl)azetidine-3-carboxamide formic acid (compound D8 formic acid)

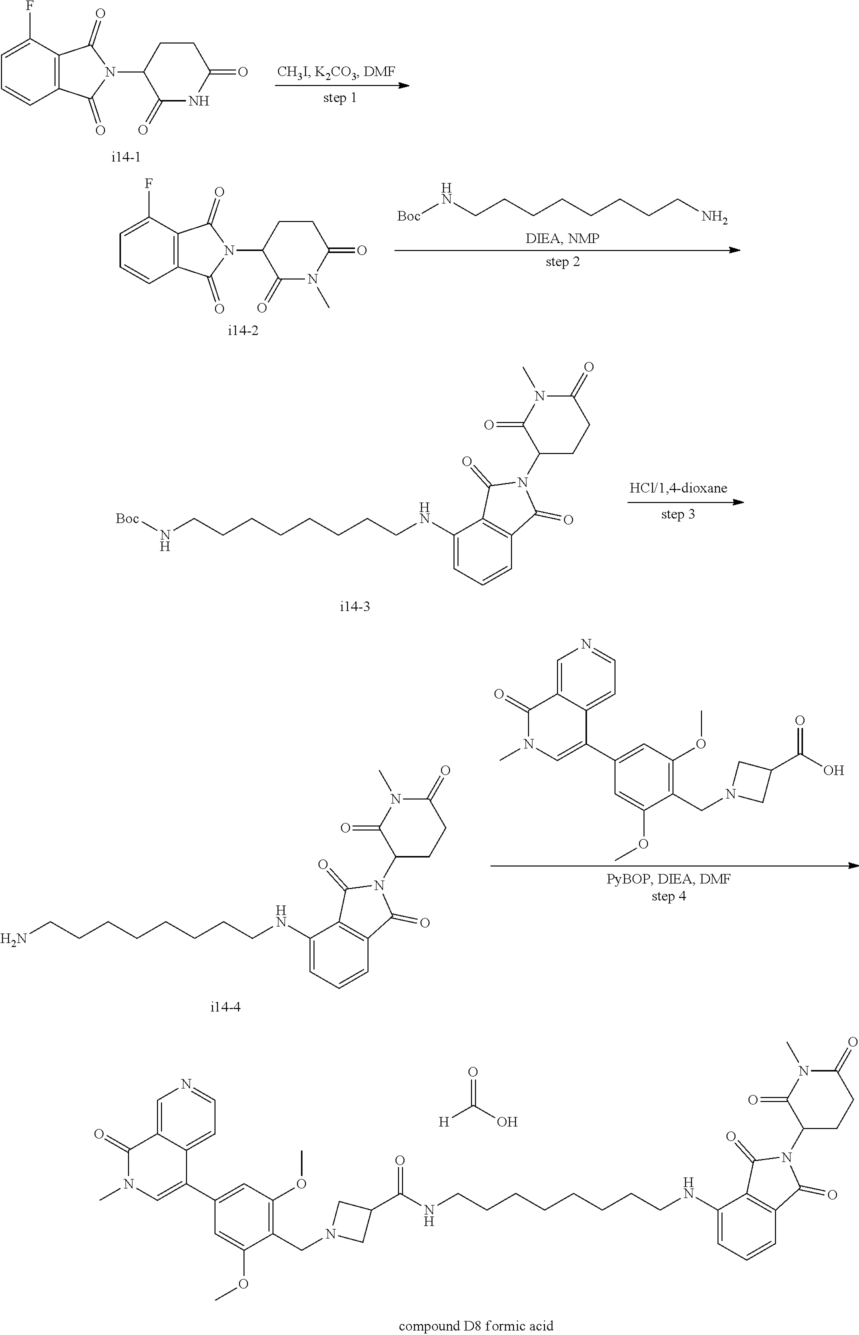

### Step 1: Preparation of 4-fluoro-2-(1-methyl-2,6-dioxopiperidin-3-yl)-2,3-dihydro-1H-isoindole-1,3-dione (i14-2)

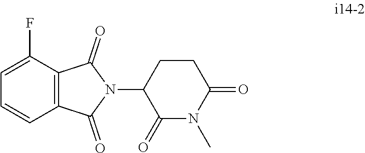 [SMILES: CN1C(=O)CCC(N2C(=O)c3cccc(F)c3C2=O)C1=O]

To a solution of 2-(2,6-dioxopiperidin-3-yl)-4-fluoro-2,3-dihydro-1H-isoindole-1,3-dione (500 mg, 1.810 mmol, 1 equiv) in DMF (10 mL) was added CH₃I (385.39 mg, 2.715 mmol, 1.5 equiv) and K₂CO₃ (750.51 mg, 5.430 mmol, 3 equiv). The resulting solution was stirred for overnight at 25° C. The solids were filtered out. The resulting mixture was concentrated. The residue was applied onto a silica gel column with ethyl acetate/petroleum ether (1:2). This resulted in 4-fluoro-2-(1-methyl-2,6-dioxopiperidin-3-yl)-2,3-dihydro-1H-isoindole-1,3-dione (480 mg, 91.36%) as a white solid. LCMS (ESI) m/z: [M−H]+=291.

### Step 2: Preparation of tert-butyl N-(8-[[2-(1-methyl-2,6-dioxopiperidin-3-yl)-1,3-dioxo-2,3-dihydro-1H-isoindol-4-yl]amino]octyl)carbamate (i14-3)

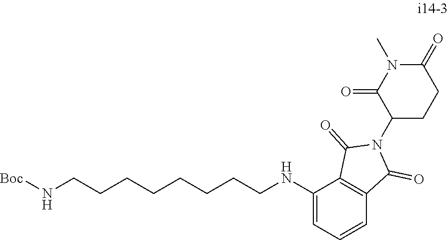 [SMILES: CN1C(=O)CCC(N2C(=O)c3cccc(NCCCCCCCCNC(=O)OC(C)(C)C)c3C2=O)C1=O]

To a solution of 4-fluoro-2-(1-methyl-2,6-dioxopiperidin-3-yl)-2,3-dihydro-1H-isoindole-1,3-dione (480 mg, 1.654 mmol, 1 equiv) and tert-butyl N-(8-aminooctyl)carbamate (404.14 mg, 1.654 mmol, 1 equiv) in NMP (10 mL) was added DIEA (641.21 mg, 4.961 mmol, 3 equiv). The resulting solution was stirred for 6 hours at 90° C. The resulting solution was diluted with 20 mL of water and extracted with ethyl acetate (2×20 mL), and the organic layers were combined and dried over anhydrous sodium sulfate and concentrated. The residue was applied onto a silica gel column with ethyl acetate/petroleum ether (1:1). This resulted in tert-butyl N-(8-[[2-(1-methyl-2,6-dioxopiperidin-3-yl)-1,3-dioxo-2,3-dihydro-1H-isoindol-4-yl]amino]octyl)carbamate (480 mg, 56.40%) as a green solid. LCMS (ESI) m/z: [M−H]+=515.

### Step 3: Preparation of 4-[(8-aminooctyl)amino]-2-(1-methyl-2,6-dioxopiperidin-3-yl)-2,3-dihydro-1H-isoindole-1,3-dione (i14-4)

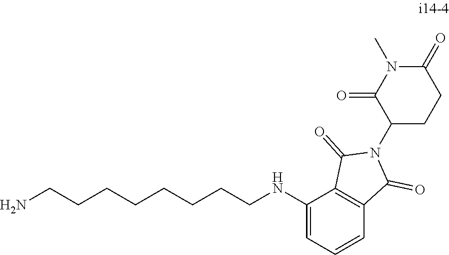 [SMILES: CN1C(=O)CCC(N2C(=O)c3cccc(NCCCCCCCCN)c3C2=O)C1=O]

A mixture of tert-butyl N-(8-[[2-(1-methyl-2,6-dioxopiperidin-3-yl)-1,3-dioxo-2,3-dihydro-1H-isoindol-4-yl]amino]octyl)carbamate (i50 mg, 0.291 mmol, 1 equiv) and 4 M HCl in 1,4-dioxane (5 mL) was stirred for 1 hour at 25° C. The resulting mixture was concentrated. This resulted in 4-[(8-aminooctyl)amino]-2-(1-methyl-2,6-dioxopiperidin-3-yl)-2,3-dihydro-1H-isoindole-1,3-dione (100 mg, 82.77%) as a white solid, that was used directly without further purification. LCMS (ESI) m/z: [M−H]+=415.

### Step 4: Preparation of 1-[[2,6-dimethoxy-4-(2-methyl-1-oxo-1,2-dihydro-2,7-naphthyridin-4-yl)phenyl]methyl]-N-(8-[[2-(1-methyl-2,6-dioxopiperidin-3-yl)-1,3-dioxo-2,3-dihydro-1H-isoindol-4-yl]amino]octyl)azetidine-3-carboxamide formic acid (compound D8 formic acid)

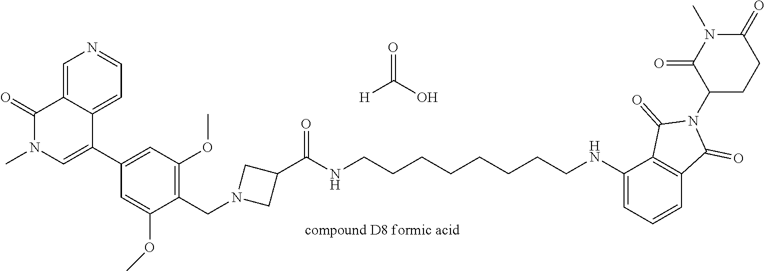 [SMILES: COc1cc(-c2cn(C)c(=O)c3cnccc23)cc(OC)c1CN1CC(C(=O)NCCCCCCCCNc2cccc3c2C(=O)N(C2CCC(=O)N(C)C2=O)C3=O)C1 ; O=CO]

To a solution of 4-[(8-aminooctyl)amino]-2-(1-methyl-2,6-dioxopiperidin-3-yl)-2,3-dihydro-1H-isoindole-1,3-dione (80 mg, 0.193 mmol, 1 equiv) and 1-[[2,6-dimethoxy-4-(2-methyl-1-oxo-1,2-dihydro-2,7-naphthyridin-4-yl)phenyl]methyl]azetidine-3-carboxylic acid (79.02 mg, 0.193 mmol, 1 equiv) in DMF (3 mL) was added HATU (110.08 mg, 0.290 mmol, 1.5 equiv) and DIEA (49.89 mg, 0.386 mmol, 2 equiv). The resulting solution was stirred for 2 hours at 25° C. The crude product was purified by Prep-HPLC (conditions: XBridge Prep C18 OBD Column, 5 μm, 19*150 mm; mobile phase, Water (0.1% FA) and ACN; Detector, UV 254 nm). This resulted in 1-[[2,6-dimethoxy-4-(2-methyl-1-oxo-1,2-dihydro-2,7-naphthyridin-4-yl)phenyl]methyl]-N-(8-[[2-(1-methyl-2,6-dioxopiperidin-3-yl)-1,3-dioxo-2,3-dihydro-1H-isoindol-4-yl]amino]octyl)azetidine-3-carboxamide (15 mg, 9.64%) as a yellow solid. ¹H NMR (400 MHz, Methanol-d4) δ 9.54 (d, J=0.8 Hz, 1H), 8.69 (d, J=5.8 Hz, 1H), 8.54 (s, 1.2H, FA), 7.77 (s, 1H), 7.65-7.52 (m, 2H), 7.10-7.01 (m, 2H), 6.84 (s, 2H), 5.10 (dd, J=12.9, 5.4 Hz, 1H), 4.39 (s, 2H), 4.14 (d, J=8.2 Hz, 3H), 3.95 (s, 6H), 3.71 (s, 3H), 3.54 (d, J=8.1 Hz, 1H), 3.22 (t, J=7.0 Hz, 2H), 3.17 (d, J=3.1 Hz, 1H), 3.15 (s, 3H), 2.99 (s, 1H), 2.96-2.86 (m, 2H), 2.69 (dt, J=12.7, 6.3 Hz, 2H), 2.15-2.05 (m, 1H), 1.68 (p, J=7.1 Hz, 2H), 1.52 (q, J=7.1 Hz, 2H), 1.38 (s, 8H). LCMS (ESI) m/z: [M−H]⁺=806.40.

### Example 15—Preparation of 2-(1-[[2,6-dimethoxy-4-(2-methyl-1-oxo-1,2-dihydro-2,7-naphthyridin-4-yl)phenyl]methyl]azetidin-3-yl)-N-(8-[[2-(2,6-dioxopiperidin-3-yl)-1,3-dioxo-2,3-dihydro-1H-isoindol-4-yl]amino]octyl)acetamide formic acid (compound D9 formic acid)

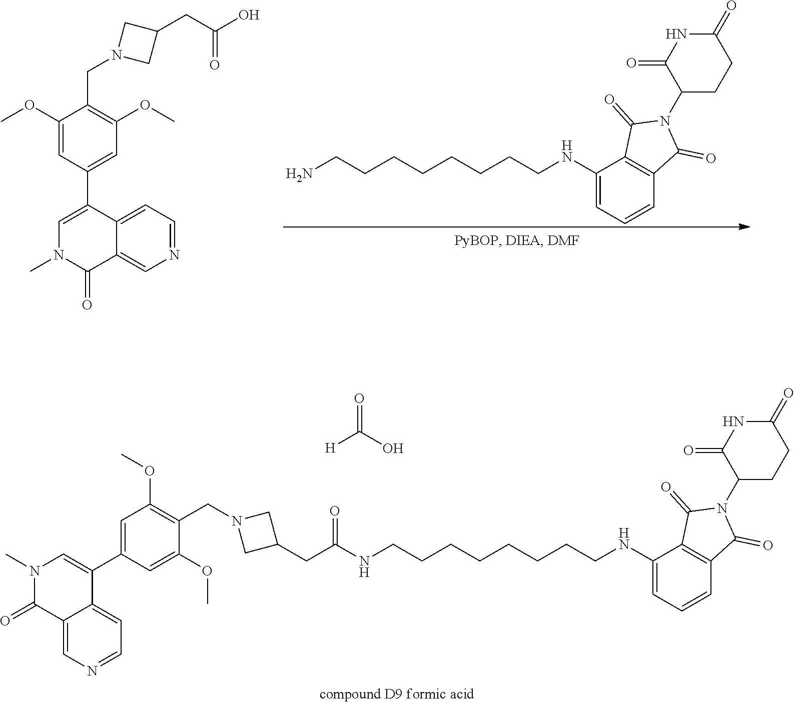 [SMILES: COc1cc(-c2cn(C)c(=O)c3cnccc23)cc(OC)c1CN1CC(CC(=O)O)C1 ; NCCCCCCCCNc1cccc2c1C(=O)N(C1CCC(=O)NC1=O)C2=O ; COc1cc(-c2cn(C)c(=O)c3cnccc23)cc(OC)c1CN1CC(CC(=O)NCCCCCCCCNc2cccc3c2C(=O)N(C2CCC(=O)NC2=O)C3=O)C1 ; O=CO]

To a solution of 2-(1-[[2,6-dimethoxy-4-(2-methyl-1-oxo-1,2-dihydro-2,7-naphthyridin-4-yl)phenyl]methyl]azetidin-3-yl)acetic acid (110 mg, 0.260 mmol, 1 equiv) in DMF (3 mL) was added 4-[(8-aminooctyl)amino]-2-(2,6-dioxopiperidin-3-yl)-2,3-dihydro-1H-isoindole-1,3-dione (104.03 mg, 0.260 mmol, 1.00 equiv), PyBOP (202.77 mg, 0.390 mmol, 1.50 equiv), and DIEA (167.86 mg, 1.299 mmol, 5.00 equiv). The resulting mixture was stirred at room temperature for 16 hours. Without workup, the crude product was purified by Prep-HPLC (conditions: SunFire C18 OBD Prep Column, 100A, 5 μm, 19 mm×250 mm; Mobile Phase A: Water (0.1% FA), Mobile Phase B: ACN; Flow rate: 25 mL/minute; Gradient: 27% B to 34% B in 8 minutes; 254 nm; Rt: 6.28 minutes) to afford 2-(1-[[2,6-dimethoxy-4-(2-methyl-1-oxo-1,2-dihydro-2,7-naphthyridin-4-yl)phenyl]methyl]azetidin-3-yl)-N-(8-[[2-(2,6-dioxopiperidin-3-yl)-1,3-dioxo-2,3-dihydro-1H-isoindol-4-yl]amino]octyl)acetamide formic acid (26.7 mg) as a yellow solid. ¹H NMR (300 MHz, Methanol-d4) δ 9.52 (s, 1H), 8.69 (d, J=5.8 Hz, 1H), 8.56 (s, 0.8H, FA), 7.76 (s, 1H), 7.61 (d, J=5.7 Hz, 1H), 7.54 (dd, J=8.5, 7.1 Hz, 1H), 7.03 (dd, J=7.8, 3.5 Hz, 2H), 6.85 (s, 2H), 5.06 (dd, J=12.4, 5.4 Hz, 1H), 4.43 (s, 2H), 4.18 (t, J=9.5 Hz, 2H), 4.02-3.90 (m, 7H), 3.70 (s, 3H), 3.30 (d, J=6.8 Hz, 2H), 3.17 (t, J=7.1 Hz, 3H), 2.97-2.62 (m, 3H), 2.58 (d, J=7.4 Hz, 2H), 2.19-2.05 (m, 1H), 1.65 (q, J=7.0 Hz, 2H), 1.57-1.37 (m, 10H). LCMS (ESI) m/z: [M+H]⁺=806.25.

### Example 16—Preparation of 1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl) azetidine-3-carboxamide (D10)

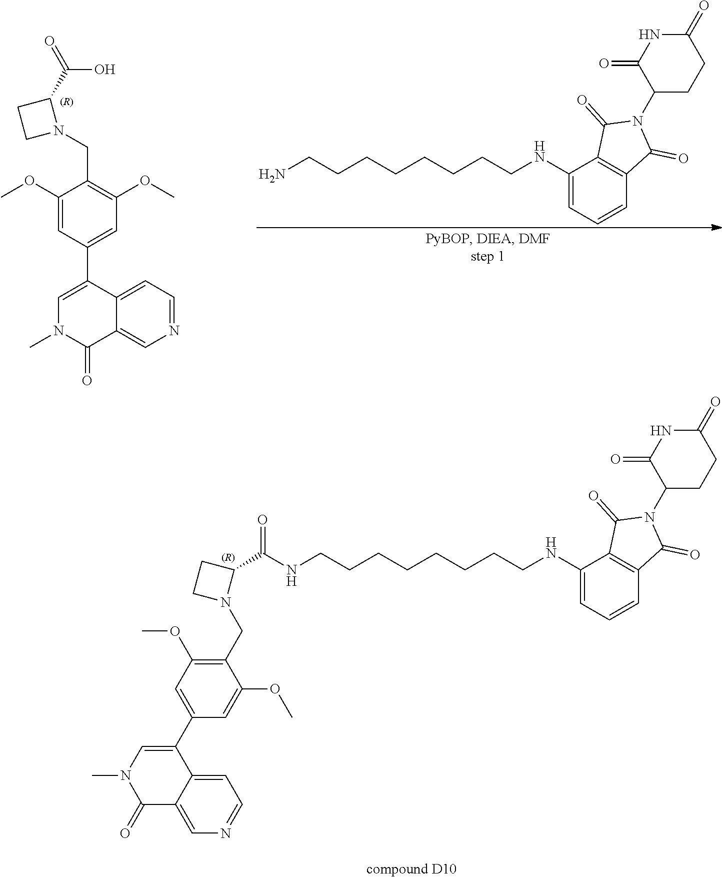

To a stirred solution of (R)-1-[[2,6-dimethoxy-4-(2-methyl-1-oxo-1,2-dihydro-2,7-naphthyridin-4-yl)phenyl]methyl]azetidine-3-carboxylic acid (40.9 mg, 0.100 mmol, 1 equiv), DIEA (64.55 mg, 0.499 mmol, 5 equiv), and PyBOP (155.95 mg, 0.300 mmol, 3 equiv) in DMF (1 mL) was added 4-[(8-aminooctyl)amino]-2-(2,6-dioxopiperidin-3-yl)-2,3-dihydro-1H-isoindole-1,3-dione hydrochloride (43.65 mg, 0.100 mmol, 1 equiv) at ambient atmosphere. The mixture was stirred for 1 hour at room (conditions: XBridge Shield RP18 OBD Column, 5 μm, 19*150 mm; Mobile Phase A: Water (0.1% FA), Mobile Phase B: ACN; Flow rate: 25 mL/minuteute; Gradient: 18% B to 35% B in 12 minutes; 254/220 nm; Rt: 11.74 minutes) to afford (R)-1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)azetidine-3-carboxamide (25 mg, 31.60%) as a yellow solid. ¹H NMR (300 MHz, Methanol-d4) δ 9.52 (s, 1H), 8.68 (d, J=5.8 Hz, 1H), 7.74 (s, 1H), 7.62 (d, J=5.8 Hz, 1H), 7.54 (dd, J=8.5, 7.1 Hz, 1H), 7.01 (t, J=7.8 Hz, 2H), 6.78 (s, 2H), 5.06 (dd, J=12.3, 5.5 Hz, 1H), 4.17 (s, 2H), 3.93 (s, 6H), 3.97-3.82 (m, 1H), 3.74 (s, 2H), 3.69 (s, 3H), 3.31-3.09 (m, 4H), 2.97-2.62 (m, 3H), 2.50 (d, J=9.2 Hz, 1H), 2.32-2.20 (m, 1H), 2.19-2.09 (m, 1H), 1.57 (q, J=6.9 Hz, 2H), 1.45-1.30 (m, 10H). LCMS (ESI) m/z: [M+H]+=792.20.

### Example 17—Preparation of (2S)-1-[[2,6-dimethoxy-4-(2-methyl-1-oxo-2,7-naphthyridin-4-yl)phenyl]methyl]-N-(8-[[2-(2,6-dioxopiperidin-3-yl)-1,3-dioxoisoindol-4-yl]amino]octyl)azetidine-2-carboxamide (compound D11)

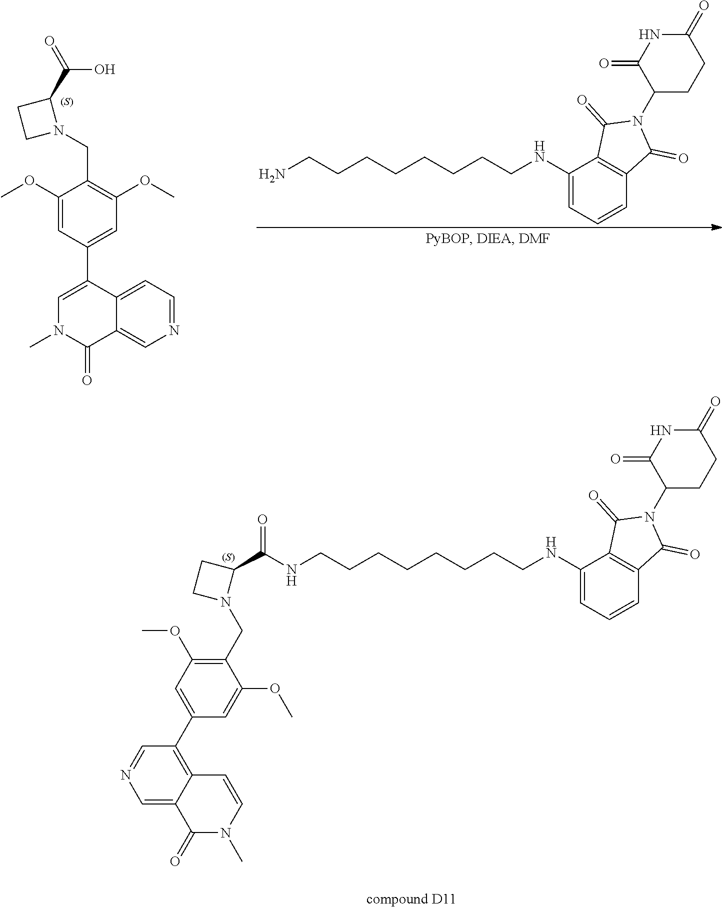

To a solution of (2S)-1-[[2,6-dimethoxy-4-(2-methyl-1-oxo-1,2-dihydro-2,7-naphthyridin-4-yl)phenyl]methyl]azetidine-2-carboxylic acid (50.00 mg, 0.122 mmol, 1.00 equiv) and 4-[(8-aminooctyl)amino]-2-(2,6-dioxopiperidin-3-yl)-2,3-dihydro-1H-isoindole-1,3-dione (48.91 mg, 0.122 mmol, 1.00 equiv) in DMF (2.00 mL) was added PyBOP (127.10 mg, 0.244 mmol, 2.00 equiv) and DIEA (47.35 mg, 0.366 mmol, 3.00 equiv). The resulting solution was stirred at 25° C. for 2 hours. The crude product was purified by preparative HPLC (condition: XSelect CSH Prep C18 OBD Column, 5 μm, 19*150 mm; Mobile Phase A: Water (0.1% FA), Mobile Phase B: ACN; Flow rate: 25 mL/minuteute; Gradient: 20% B to 55% B in 8 minutes; 254 nm; Rt: 7.12 minutes). Fractions containing the desired compound were evaporated to dryness to afford (2S)-1-[[2,6-dimethoxy-4-(2-methyl-1-oxo-2,7-naphthyridin-4-yl)phenyl]methyl]-N-(8-[[2-(2,6-dioxopiperidin-3-yl)-1,3-dioxoisoindol-4-yl]amino]octyl)azetidine-2-carboxamide (35 mg, 35.47%) as a yellow solid. ¹H NMR (400 MHz, Methanol-d4) δ 9.51 (s, 1H), 8.68 (d, J=5.7 Hz, 1H), 7.72 (s, 1H), 7.62 (d, J=5.8 Hz, 1H), 7.53 (t, J=7.8 Hz, 1H), 7.00 (dd, J=10.6, 7.8 Hz, 2H), 6.75 (s, 2H), 5.05 (dd, J=12.4, 5.4 Hz, 1H), 3.89 (s, 9H), 3.69 (s, 3H), 3.30 (s, 2H), 3.25 (t, J=6.9 Hz, 2H), 3.15 (t, J=7.1 Hz, 2H), 2.94-2.64 (m, 3H), 2.35 (d, J=9.5 Hz, 1H), 2.16-2.00 (m, 1H), 1.58 (t, J=7.1 Hz, 2H), 1.40 (d, J=6.7 Hz, 2H), 1.30 (s, 8H). LCMS (ESI) m/z: [M+H]+=792.60.

### Example 18—Preparation of 1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)azetidine-3-sulfonamide (compound D12)

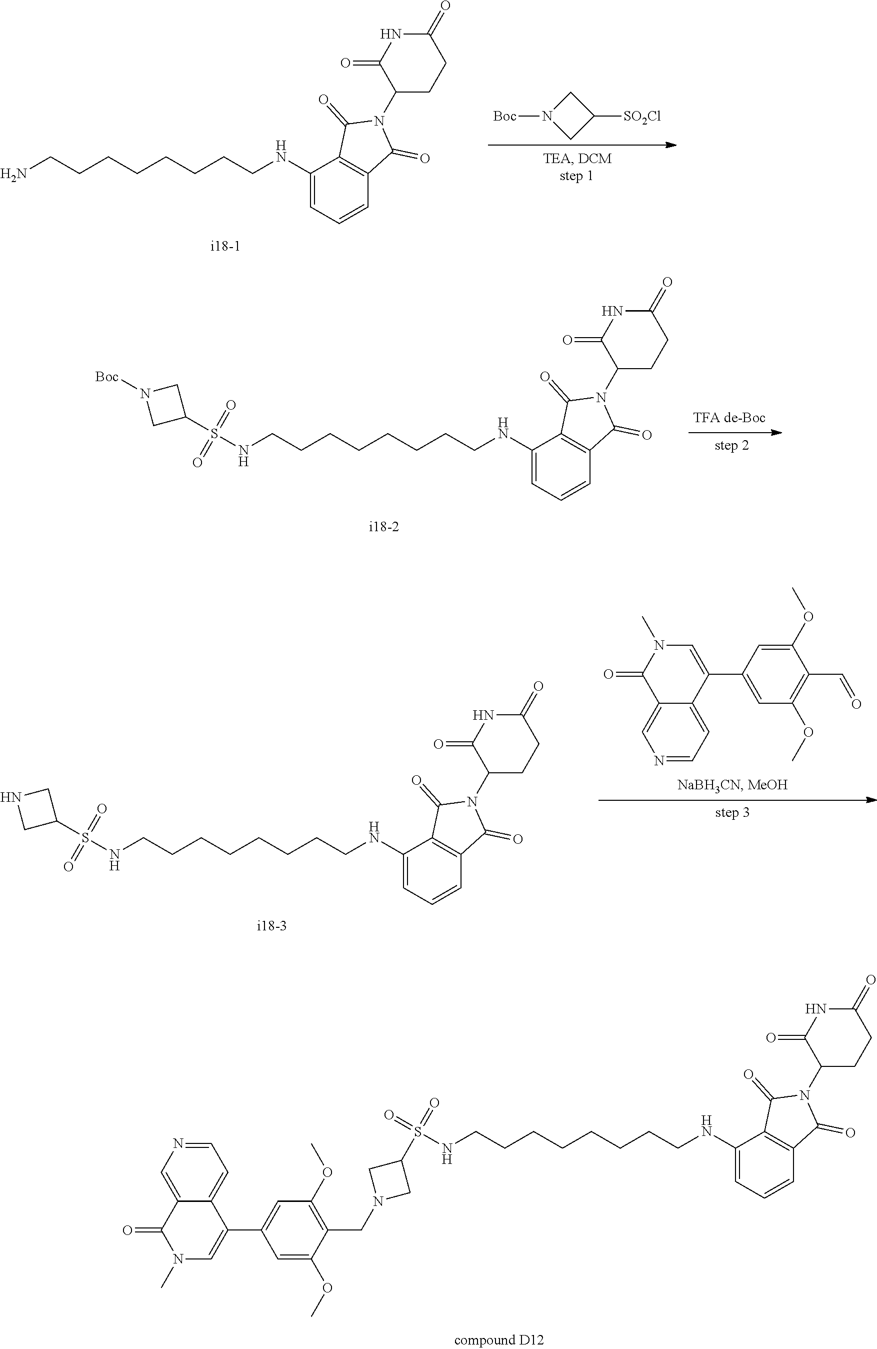 [SMILES: NCCCCCCCCNc1cccc2c1C(=O)N(C1CCC(=O)NC1=O)C2=O ; CC(C)(C)OC(=O)N1CC(S(=O)(=O)Cl)C1 ; CC(C)(C)OC(=O)N1CC(S(=O)(=O)NCCCCCCCCNc2cccc3c2C(=O)N(C2CCC(=O)NC2=O)C3=O)C1 ; O=C1CCC(N2C(=O)c3cccc(NCCCCCCCCNS(=O)(=O)C4CNC4)c3C2=O)C(=O)N1 ; COc1cc(-c2cn(C)c(=O)c3cnccc23)cc(OC)c1C=O ; COc1cc(-c2cn(C)c(=O)c3cnccc23)cc(OC)c1CN1CC(S(=O)(=O)NCCCCCCCCNc2cccc3c2C(=O)N(C2CCC(=O)NC2=O)C3=O)C1]

### Step 1: Preparation of tert-butyl 3-[(8-[[2-(2,6-dioxopiperidin-3-yl)-1,3-dioxo-2,3-dihydro-1H-isoindol-4-yl]amino]octyl)sulfamoyl]azetidine-1-carboxylate (i18-2)

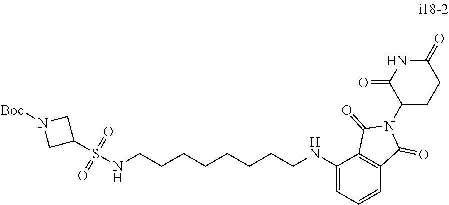 [SMILES: CC(C)(C)OC(=O)N1CC(S(=O)(=O)NCCCCCCCCNc2cccc3c2C(=O)N(C2CCC(=O)NC2=O)C3=O)C1]

To a solution of 4-[(8-aminooctyl)amino]-2-(2,6-dioxopiperidin-3-yl)-2,3-dihydro-1H-isoindole-1,3-dione (100.00 mg, 0.250 mmol, 1.00 equiv) in DCM (2.00 mL) was added tert-butyl 3-(chlorosulfonyl)azetidine-1-carboxylate (95.78 mg, 0.375 mmol, 1.50 equiv) and TEA (50.53 mg, 0.499 mmol, 2.00 equiv) at 0° C. The resulting solution was stirred for 2 hours at 25° C. The reaction was then quenched by the addition of 5 mL of MeOH. The resulting mixture was concentrated. The residue was applied onto a silica gel column with ethyl DCM/MeOH (20:1). This resulted in 110 mg (71.08%) of tert-butyl 3-[(8-[[2-(2,6-dioxopiperidin-3-yl)-1,3-dioxo-2,3-dihydro-1H-isoindol-4-yl]amino]octyl) sulfamoyl]azetidine-1-carboxylate as a yellow solid. LCMS (ESI) m/z: [M+H]+=620.

### Step 2: Preparation of N-(8-[[2-(2,6-dioxopiperidin-3-yl)-1,3-dioxoisoindol-4-yl]amino]octyl)azetidine-3-sulfonamide (i18-3)

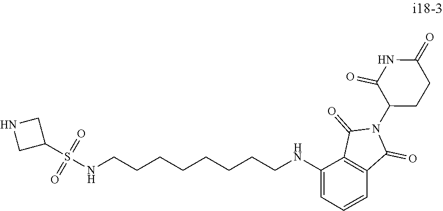 [SMILES: O=C1CCC(N2C(=O)c3cccc(NCCCCCCCCNS(=O)(=O)C4CNC4)c3C2=O)C(=O)N1]

A solution of tert-butyl 3-[(8-[[2-(2,6-dioxopiperidin-3-yl)-1,3-dioxoisoindol-4-yl]amino]octyl) sulfamoyl]azetidine-1-carboxylate (110.00 mg, 0.177 mmol, 1.00 equiv) in TFA (2.00 mL) and CH₂Cl₂ (2.00 mL) was stirred at 0° C. for 1 hour. The resulting mixture was concentrated under reduced pressure to afford N-(8-[[2-(2,6-dioxopiperidin-3-yl)-1,3-dioxoisoindol-4-yl]amino]octyl)azetidine-3-sulfonamide (85 mg, 92.16%) as a yellow solid, which was used directly without further purification. LCMS (ESI) m/z: [M+H]+=520.

### Step 3: Preparation of 1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)azetidine-3-sulfonamide (compound D12)

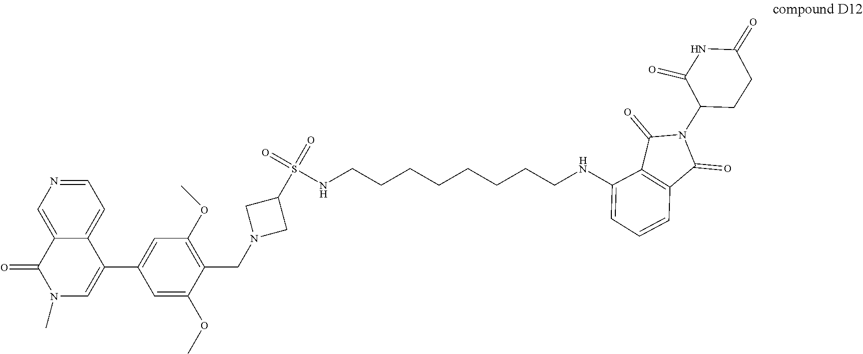 [SMILES: COc1cc(-c2cn(C)c(=O)c3cnccc23)cc(OC)c1CN1CC(S(=O)(=O)NCCCCCCCCNc2cccc3c2C(=O)N(C2CCC(=O)NC2=O)C3=O)C1]

To a solution of N-(8-[[2-(2,6-dioxopiperidin-3-yl)-1,3-dioxo-2,3-dihydro-1H-isoindol-4-yl]amino]octyl)azetidine-3-sulfonamide (85.00 mg, 0.164 mmol, 1.00 equiv) and 2,6-dimethoxy-4-(2-methyl-1-oxo-1,2-dihydro-2,7-naphthyridin-4-yl)benzaldehyde (53.06 mg, 0.164 mmol, 1.00 equiv) in MeOH (2.00 mL) was added NaBH₃CN (20.56 mg, 0.327 mmol, 2.00 equiv). The resulting solution was stirred at 25° C. for 2 hours. The crude product was purified by preparative HPLC Column (condition: XSelect CSH Prep C18 OBD Column, 5 μm, 19*150 mm; Mobile Phase A: Water (0.1% FA), Mobile Phase B: ACN; Flow rate: 25 mL/minutes; Gradient: 20% B to 55% B in 8 minutes; 254 nm; Rt: 7.12 minutes). Fractions containing the desired compound were evaporated to dryness to afford 1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)azetidine-3-sulfonamide(50 mg, 36.92%) as a white solid. ¹H NMR (400 MHz, Methanol-d4) δ 9.52 (d, J=0.9 Hz, 1H), 8.68 (d, J=5.7 Hz, 1H), 8.53 (s, 0.47H, FA), 7.74 (s, 1H), 7.63 (dd, J=5.8, 0.9 Hz, 1H), 7.55 (dd, J=8.5, 7.1 Hz, 1H), 7.03 (dd, J=7.8, 4.8 Hz, 2H), 6.77 (s, 2H), 5.06 (dd, J=12.5, 5.5 Hz, 1H), 4.03 (p, J=8.2, 7.8 Hz, 1H), 3.91 (d, J=4.1 Hz, 2H), 3.89 (s, 6H), 3.78-3.68 (m, 8H), 3.30 (d, J=6.8 Hz, 1H), 3.03 (t, J=7.0 Hz, 2H), 2.94-2.80 (m, 1H), 2.80-2.66 (m, 2H), 2.17-2.08 (m, 1H), 1.70-1.62 (m, 2H), 1.51 (d, J=6.9 Hz, 2H), 1.44-1.37 (m, 8H). LCMS (ESI) m/z: [M+H]+=828.35.

### Example 19—Preparation of 1-(2,6-dimethoxy-4-(2-methyl-1-oxo-1,2-dihydro-2,7-naphthyridin-4-yl)benzyl)-N-(8-((2-(2,6-dioxopiperidin-3-yl)-1,3-dioxoisoindolin-4-yl)amino)octyl)-3-methylazetidine-3-carboxamide (compound D13)

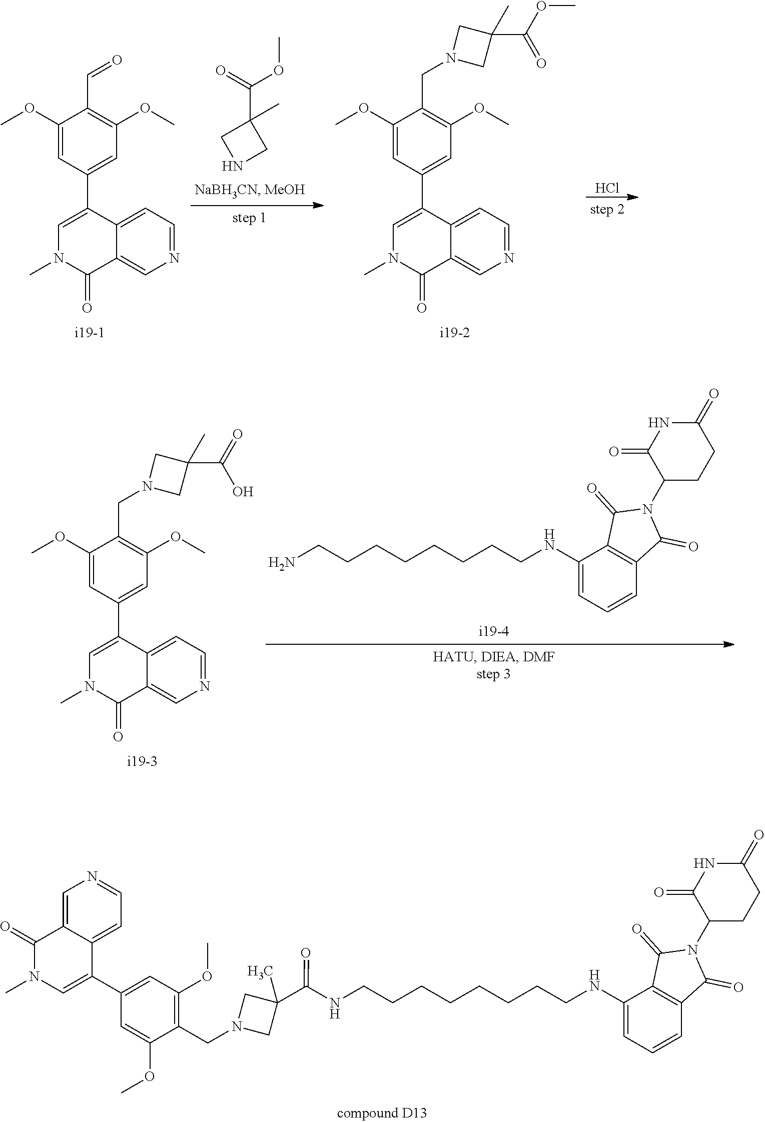

### Step 1: Preparation of methyl 1-(2,6-dimethoxy-4-(2-methyl-1-oxo-1,2-dihydro-2,7-naphthyridin-4-yl)benzyl)-3-methylazetidine-3-carboxylate (i19-2)

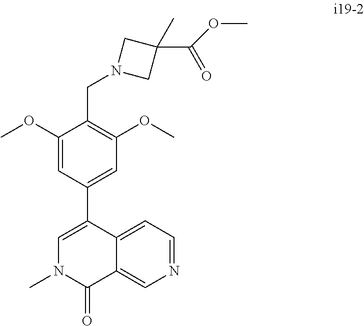

To a solution of 2,6-dimethoxy-4-(2-methyl-1-oxo-1,2-dihydro-2,7-naphthyridin-4-yl)benzaldehyde (200 mg, 0.617 mmol, 1 equiv) and methyl 3-methylazetidine-3-carboxylate (79.65 mg, 0.617 mmol, 1.00 equiv) in MeOH (2 mL) was added NaBH₃CN (77.50 mg, 1.233 mmol, 2 equiv). The resulting solution was stirred at 25° C. for 1 hour. The residue was purified by silica gel column chromatography, eluted with DCM/MeOH (9:1) to afford methyl 1-[[2, 6-dimethoxy-4-(2-methyl-1-oxo-1,2-dihydro-2,7-naphthyridin-4-yl) phenyl]methyl]-3-methylazetidine-3-carboxylate (247 mg, 91.56%) as a yellow solid. LCMS (ESI) m/z: [M+H]+=438.

### Step 2: Preparation of 1-(2,6-dimethoxy-4-(2-methyl-1-oxo-1,2-dihydro-2,7-naphthyridin-4-yl)benzyl)-3-methylazetidine-3-carboxylic acid (i19-3)

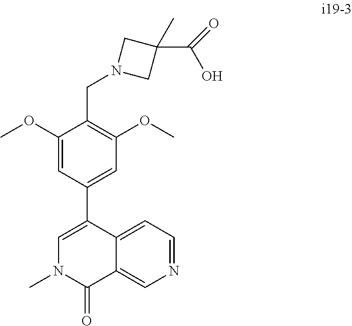 [SMILES: COc1cc(-c2cn(C)c(=O)c3cnccc23)cc(OC)c1CN1CC(C)(C(=O)O)C1]

A solution of methyl 1-[[2,6-dimethoxy-4-(2-methyl-1-oxo-1,2-dihydro-2,7-naphthyridin-4-yl)phenyl]methyl]-3-methylazetidine-3-carboxylate (235 mg, 0.537 mmol, 1 equiv) in HCl (12 M, 5 mL) was stirred at 25° C. for 40 minutes. The mixture was concentrated under reduced pressure afford 1-[[2,6-dimethoxy-4-(2-methyl-1-oxo-1,2-dihydro-2,7-naphthyridin-4-yl)phenyl]methyl]-3-methylazeti-dine-3-carboxylic acid (185 mg, 81.33%) as a brown solid, that was used directly without further purification. LCMS (ESI) m/z: [M+H]+=424.

### Step 3: Preparation of 1-(2,6-dimethoxy-4-(2-methyl-1-oxo-1,2-dihydro-2,7-naphthyridin-4-yl)benzyl)-N-(8-((2-(2,6-dioxopiperidin-3-yl)-1,3-dioxoisoindolin-4-yl)amino)octyl)-3-methylazetidine-3-carboxamide (compound D13)

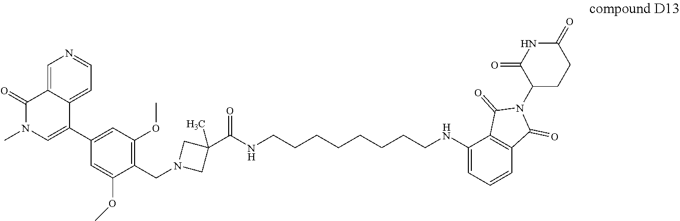 [SMILES: COc1cc(-c2cn(C)c(=O)c3cnccc23)cc(OC)c1CN1CC(C)(C(=O)NCCCCCCCCNc2cccc3c2C(=O)N(C2CCC(=O)NC2=O)C3=O)C1]

To a solution of 1-[[2,6-dimethoxy-4-(2-methyl-1-oxo-1,2-dihydro-2,7-naphthyridin-4-yl)phenyl]methyl]-3-methylazetidine-3-carboxylic acid (50 mg, 0.118 mmol, 1 equiv), 4-[(8-aminooctyl)amino]-2-(2,6-dioxopiperidin-3-yl)-2,3-dihydro-1H-isoindole-1,3-dione (94.57 mg, 0.236 mmol, 2 equiv) and Et₃N (119.48 mg, 1.181 mmol, 10.00 equiv) in DMF (3 mL), was added EDCl (27.16 mg, 0.142 mmol, 1.2 equiv) and HOBT (19.15 mg, 0.142 mmol, 1.2 equiv), the resulting solution was stirred at 25° C. for 24 hours. The crude product was purified by Prep-HPLC with the following conditions (condition: XBridge Prep C18 OBD Column, 5 μm, 19*150 mm; mobile phase, Water (0.1% FA) and ACN; Detector, UV) to give 1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)-3-methylazetidine-3-carboxamide (21.7 mg, 22.80%) as a yellow solid. ¹H NMR (300 MHz, Methanol-d4) δ 9.53 (s, 1H), 8.69 (d, J=5.7 Hz, 1H), 8.55 (s, 1H), 7.76 (s, 1H), 7.62 (d, J=5.7 Hz, 1H), 7.59-7.49 (m, 1H), 7.07-6.98 (m, 2H), 6.81 (s, 2H), 5.06 (dd, J=12.3, 5.4 Hz, 1H), 4.19 (s, 2H), 4.06 (s, 2H), 3.93 (s, 6H), 3.71 (s, 5H), 3.32-3.16 (m, 1H), 2.92-2.66 (m, 4H), 2.15-2.06 (m, 1H), 1.64 (d, J=7.4 Hz, 2H), 1.55 (s, 5H), 1.39-1.32 (m, 8H). LCMS (ESI) m/z: [M+H]+=806.50.

### Example 20—Preparation of 1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)-N-methylazetidine-3-carboxamide (compound D14)

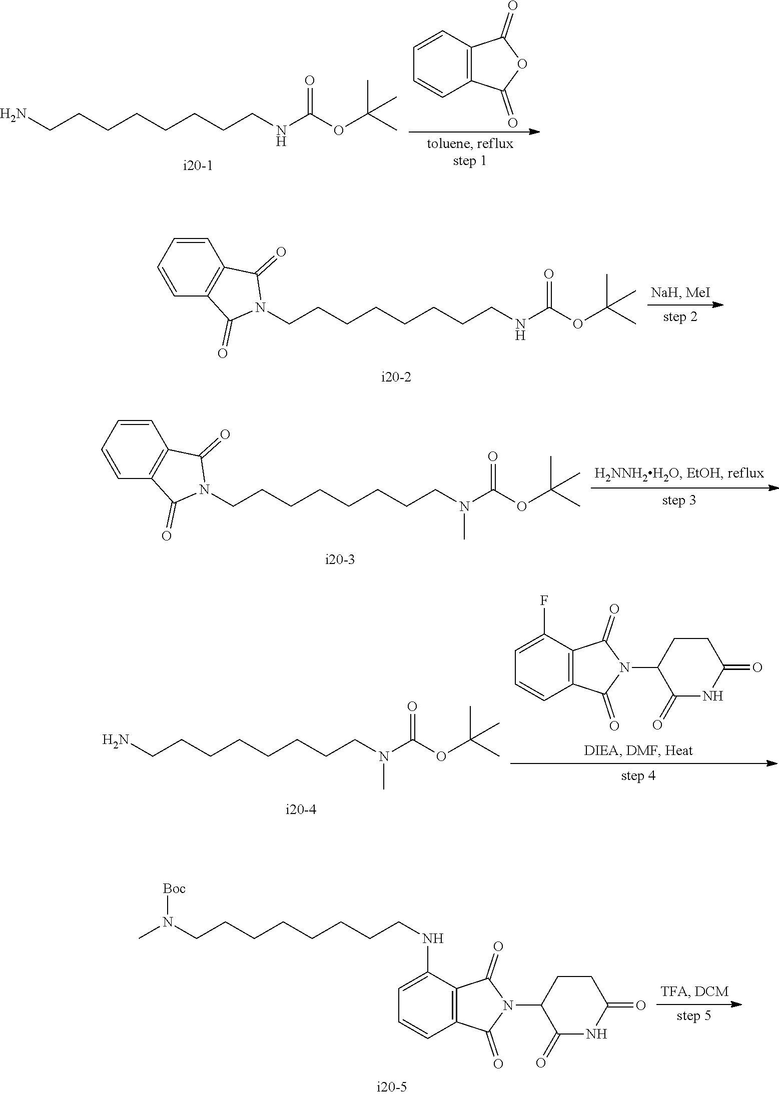

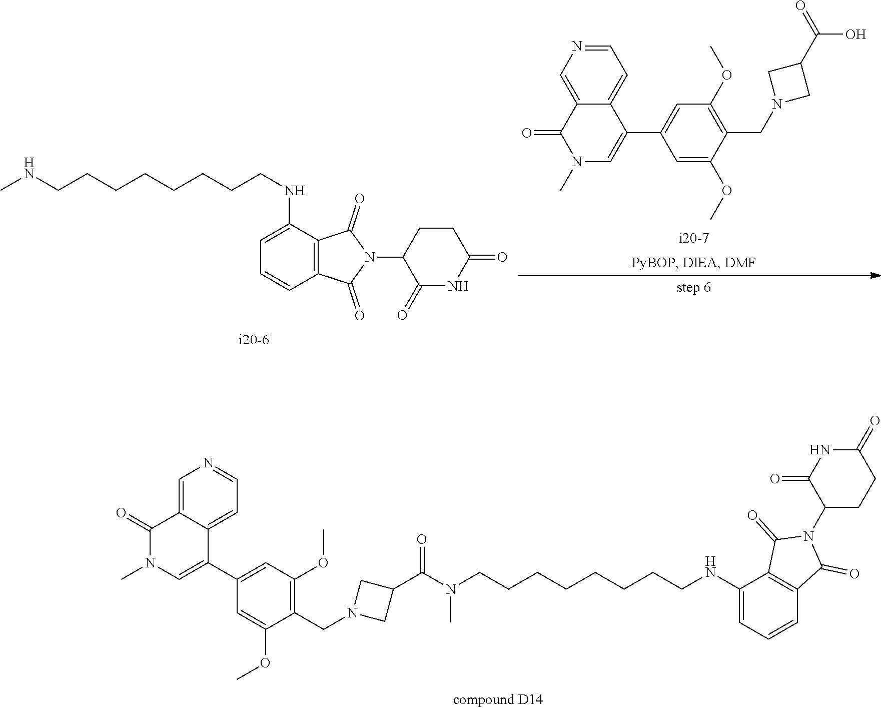 [SMILES: CNCCCCCCCCNc1cccc2c1C(=O)N(C1CCC(=O)NC1=O)C2=O ; COc1cc(-c2cn(C)c(=O)c3cnccc23)cc(OC)c1CN1CC(C(=O)O)C1 ; COc1cc(-c2cn(C)c(=O)c3cnccc23)cc(OC)c1CN1CC(C(=O)N(C)CCCCCCCCNc2cccc3c2C(=O)N(C2CCC(=O)NC2=O)C3=O)C1]

### Step 1: preparation of tert-butyl N-[8-(1,3-dioxoisoindol-2-yl)octyl]carbamate (i20-2)

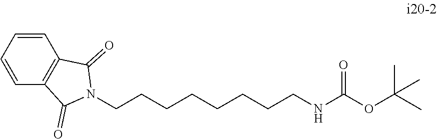 [SMILES: CC(C)(C)OC(=O)NCCCCCCCCN1C(=O)c2ccccc2C1=O]

A mixture of tert-butyl N-(8-aminooctyl)carbamate (1.00 g, 4.092 mmol, 1.00 equiv) and phthalic anhydride (606.10 mg, 4.092 mmol, 1.00 equiv) in toluene (20.00 mL) was stirred for 2 hours at 130° C. under nitrogen atmosphere. The mixture was allowed to cool down to room temperature and the solvent was evaporated. The resulting residue was purified by silica gel column chromatography, eluted with PE/EtOAc (10:1) to afford tert-butyl N-[8-(1,3-dioxoisoindol-2-yl)octyl]carbamate (1.7 g, 95.41%) as a white solid. LCMS (ESI) m/z: [M+H]+=375.

### Step 2: Preparation of tert-butyl N-[8-(1,3-dioxoisoindol-2-yl)octyl]-N-methylcarbamate (i20-3)

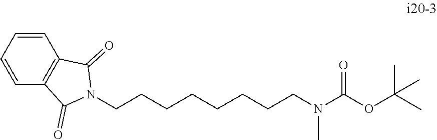 [SMILES: CN(CCCCCCCCN1C(=O)c2ccccc2C1=O)C(=O)OC(C)(C)C]

To a stirred solution of tert-butyl N-[8-(1,3-dioxoisoindol-2-yl)octyl]carbamate(1.24 g, 3.311 mmol, 1.00 equiv) in DMF (1.00 mL) was added NaH (0.16 g, 6.622 mmol, 2 equiv) in portions at 0° C. under nitrogen atmosphere. Then CH₃I (1.88 g, 13.245 mmol, 4 equiv) was added. The resulting mixture was stirred for 1 hour at room temperature under nitrogen atmosphere. The residue was purified by silica gel column chromatography, eluted with PE/EtOAc (12:1) to afford tert-butyl N-[8-(1,3-dioxoisoindol-2-yl)octyl]-N-methylcarbamate (800 mg, 62.19%) as a colorless liquid. LCMS (ESI) m/z: [M+H]+=389.

### Step 3: Preparation of tert-butyl N-(8-aminooctyl)-N-methylcarbamate (i20-4)

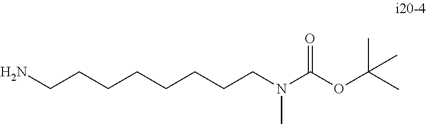 [SMILES: CN(CCCCCCCCN)C(=O)OC(C)(C)C]

A solution of tert-butyl N-[8-(1,3-dioxoisoindol-2-yl)octyl]-N-methylcarbamate (700.00 mg, 1.802 mmol, 1.00 equiv) and NH₂NH₂ (259.84 mg, 3.604 mmol, 2 equiv) in EtOH (5.00 mL) was stirred for 1 hour at 90° C. under nitrogen atmosphere. The mixture was allowed to cool down to room temperature. After filtration, the filtrate was concentrated under reduced pressure. The resulting residue was purified by silica gel column chromatography, eluted with PE/EtOAc (12:1) to afford tert-butyl N-(8-aminooctyl)-N-methylcarbamate (580 mg, 94.68%) as a colorless liquid. LCMS (ESI) m/z: [M+H]+=259.

### Step 4: Preparation of tert-butyl N-(8-[[2-(2,6-dioxopiperidin-3-yl)-1,3-dioxoisoindol-4-yl]amino]octyl)-N-methylcarbamate (i20-5)

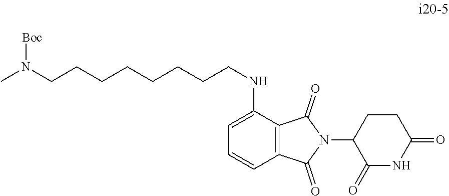 [SMILES: CN(CCCCCCCCNc1cccc2c1C(=O)N(C1CCC(=O)NC1=O)C2=O)C(=O)OC(C)(C)C]

To a stirred solution of 2-(2,6-dioxopiperidin-3-yl)-4-fluoroisoindole-1,3-dione(520.00 mg, 1.883 mmol, 1.00 equiv) and tert-butyl N-(8-aminooctyl)-N-methylcarbamate (486.46 mg, 1.883 mmol, 1 equiv) in DMF (5.00 mL) was added DIPEA (1216.53 mg, 9.413 mmol, 5 equiv). The solution was stirred for 1 hour at 90° C. under nitrogen atmosphere, then it was cooled down to room temperature. The resulting mixture was concentrated under reduced pressure. The residue was purified by silica gel column chromatography, eluted with CH₂Cl₂/MeOH (12:1) to afford tert-butyl N-(8-[[2-(2,6-dioxopiperidin-3-yl)-1,3-dioxoisoindol-4-yl]amino]octyl)-N-methylcarbamate (260 mg, 26.84%) as a yellow solid. LCMS (ESI) m/z: [M+H]+=515.

### Step 5: Preparation of 2-(2,6-dioxopiperidin-3-yl)-4-[[8-(methylamino)octyl]amino]isoindole-1,3-dione (i20-6)

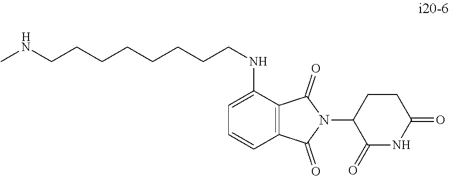 [SMILES: CNCCCCCCCCNc1cccc2c1C(=O)N(C1CCC(=O)NC1=O)C2=O]

A solution of tert-butyl N-(8-[[2-(2,6-dioxopiperidin-3-yl)-1,3-dioxoisoindol-4-yl]amino]octyl)-N-methylcarbamate (220.00 mg, 0.427 mmol, 1.00 equiv) in 4 M HCl in dioxane (6.00 mL) was stirred for 2 hours at room temperature. The solvent was evaporated and the residue was purified by reverse flash chromatography (condition: C18 silica gel column; mobile phase, MeOH in water, 10% to 50% gradient in 10 minutes; detector, UV 254 nm) to afford 2-(2,6-dioxopiperidin-3-yl)-4-[[8-(methylamino)octyl]amino]isoindole-1,3-dione(170 mg, 95.94%) as a dark yellow oil. LCMS (ESI) m/z: [M+H]+=415.

### Step 6: Preparation of 1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)-N-methylazetidine-3-carboxamide (compound D14)

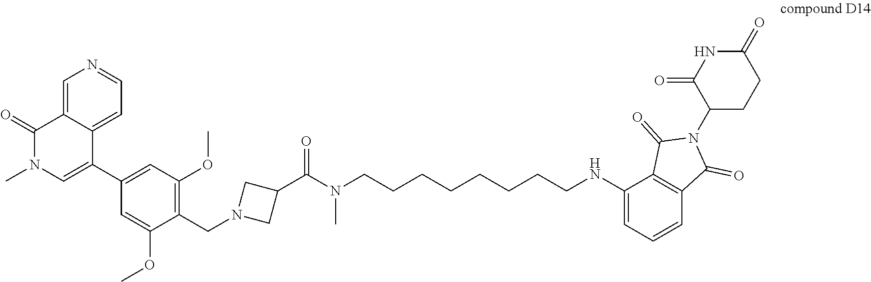 [SMILES: COc1cc(-c2cn(C)c(=O)c3cnccc23)cc(OC)c1CN1CC(C(=O)N(C)CCCCCCCCNc2cccc3c2C(=O)N(C2CCC(=O)NC2=O)C3=O)C1]

To a stirred solution of 1-[[2,6-dimethoxy-4-(2-methyl-1-oxo-1,2-dihydro-2,7-naphthyridin-4-yl)phenyl]methyl]azetidine-3-carboxylic acid (30 mg, 0.073 mmol, 1 equiv) in DMF (0.5 mL), was added DIPEA (47.35 mg, 0.366 mmol, 5 equiv), HATU (55.72 mg, 0.147 mmol, 2 equiv), and 2-(2,6-dioxopiperidin-3-yl)-4-[[8-(methylamino)octyl]amino]-2,3-dihydro-1H-isoindole-1,3-dione (30.37 mg, 0.073 mmol, 1 equiv). The reaction was stirred at ambient atmosphere for 1 hour. The mixture was purified directly by Prep-HPLC (condition: SunFire C₁₈ OBD Prep Column, 100 A, 5 μm, 19 mm×250 mm; Mobile Phase A: Water (0.1% FA), Mobile Phase B: ACN; Flow rate: 25 mL/minutes; Gradient: 24% B to 36% B in 8 minutes; 254 nm; Rt: 7.9 minutes) to afford 1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)-N-methylazetidine-3-carboxamide formate (25 mg, 40.05%) as a yellow solid. ¹H NMR (300 MHz, Methanol-d4) δ 9.52 (dd, J=4.5, 0.9 Hz, 1H), 8.68 (dd, J=5.8, 2.5 Hz, 1H), 8.56 (s, 0.5H, FA), 7.75 (d, J=2.0 Hz, 1H), 7.67-7.58 (m, 1H), 7.53 (ddd, J=8.5, 7.1, 4.7 Hz, 1H), 7.07-6.95 (m, 2H), 6.81 (d, J=1.8 Hz, 2H), 5.06 (ddd, J=12.1, 5.4, 2.5 Hz, 1H), 4.21 (d, J=4.7 Hz, 2H), 4.00 (dd, J=17.1, 8.8 Hz, 4H), 3.93 (s, 6H), 3.80 (t, J=8.2 Hz, 1H), 3.70 (d, J=3.3 Hz, 3H), 3.45-3.19 (m, 2H), 2.94 (d, J=4.3 Hz, 3H), 2.91-2.68 (m, 3H), 2.12 (s, 1H), 1.67 (s, 2H), 1.57 (d, J=6.9 Hz, 2H), 1.41-1.33 (m, 8H). LCMS (ESI) m/z: [M+H]+=806.35.

### Example 21—Preparation of 1-[[2,6-dimethoxy-4-(2-methyl-1-oxo-1,2-dihydro-2,7-naphthyridin-4-yl)phenyl]methyl]-N-(5-[[2-(2,6-dioxopiperidin-3-yl)-1,3-dioxo-2,3-dihydro-1H-isoindol-4-yl]amino]pentyl)-N-methylazetidine-3-carboxamide formic acid (compound D15 formic acid)

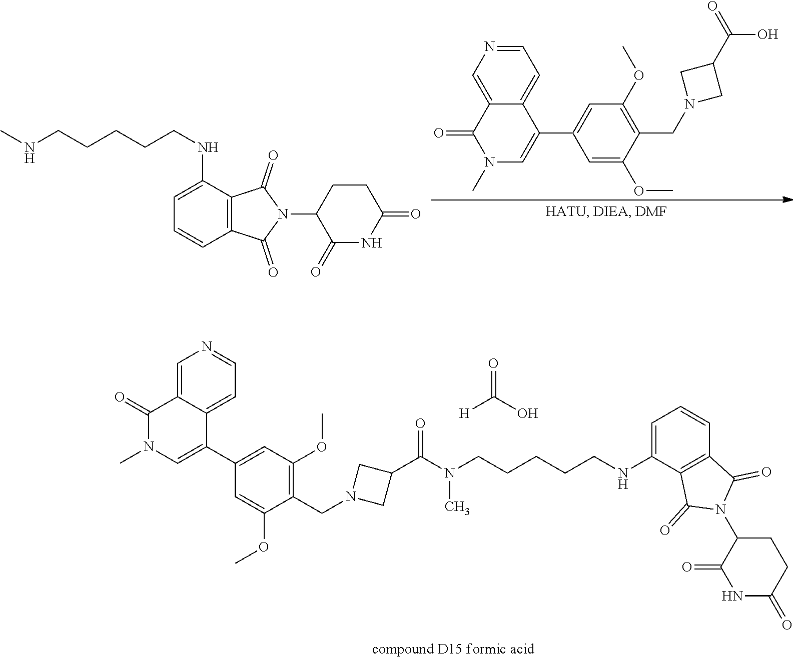

To a solution of 2-(2,6-dioxopiperidin-3-yl)-4-[[5-(methylamino)pentyl]amino]-2,3-dihydro-1H-isoindole-1,3-dione (60.00 mg, 0.161 mmol, 1.00 equiv), 1-[[2,6-dimethoxy-4-(2-methyl-1-oxo-1,2-dihydro-2,7-naphthyridin-4-yl)phenyl]methyl]azetidine-3-carboxylic acid (65.96 mg, 0.161 mmol, 1.00 equiv), and DIEA (41.64 mg, 0.322 mmol, 2.00 equiv) in DMF (2.00 mL, 25.844 mmol, 160.41 equiv) was added HATU (91.89 mg, 0.242 mmol, 1.50 equiv). The resulting mixture was stirred at room temperature for 16 hours. Without workup, the crude product was purified by Prep-HPLC (condition: XBridge Shield RP18 OBD Column 30*150 mm, 5 μm; Mobile Phase A: Water (0.1% FA), Mobile Phase B: ACN; Flow rate: 40 mL/minuteute; Gradient: 18% B to 18% B in 2 minutes; 254/220 nm; Rt: 11.43 minutes) to afford 1-[[2,6-dimethoxy-4-(2-methyl-1-oxo-1,2-dihydro-2,7-naphthyridin-4-yl)phenyl]methyl]-N-(5-[[2-(2,6-dioxopiperidin-3-yl)-1,3-dioxo-2,3-dihydro-1H-isoindol-4-yl]amino]pentyl)-N-methylazetidine-3-carboxamide; formic acid (25.1 mg) as a yellow solid. ¹H NMR (400 MHz, Methanol-d4) δ 9.53 (dd, J=5.4, 0.9 Hz, 1H), 8.68 (dd, J=5.8, 1.2 Hz, 1H), 8.56 (s, 0.53H, FA), 7.79-7.73 (m, 1H), 7.67-7.50 (m, 2H), 7.09-6.99 (m, 2H), 6.80 (d, J=3.2 Hz, 2H), 5.06 (ddd, J=12.3, 5.4, 2.8 Hz, 1H), 4.17 (s, 2H), 3.92-3.90 (m, 10H), 3.78 (q, J=9.0, 8.5 Hz, 1H), 3.71 (d, J=2.2 Hz, 3H), 3.48-3.35 (m, 2H), 3.27 (t, J=7.5 Hz, 1H), 2.98-2.85 (m, 3H), 2.89-2.64 (m, 4H), 2.22-2.08 (m, 1H), 1.75-1.62 (m, 4H), 1.43 (s, 2H). LCMS (ESI) m/z: [M+H]⁺=764.45.

### Example 22—Preparation of 2-(2,6-dihydroxypiperidin-3-yl)-4-[(8-[[hydroxy(1-[[4-(6-hydroxy-1,5-dimethyl-1,6-dihydropyridin-3-yl)-2,6-dimethoxyphenyl]methyl]azetidin-3-yl)methyl]amino]octyl)amino]-2,3-dihydro-1H-isoindole-1,3-diol formic acid (compound D16 formic acid)

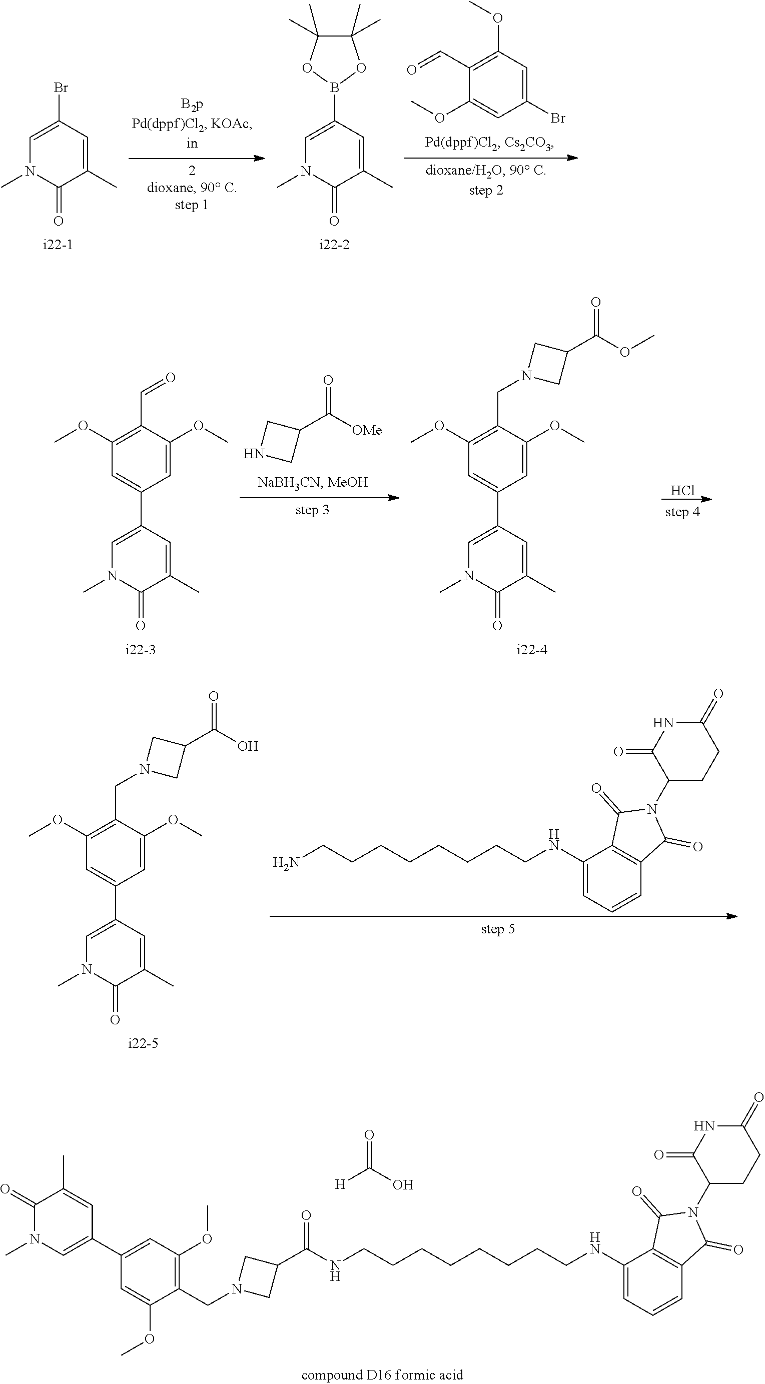

### Step 1: Preparation of 1,3-dimethyl-5-(4,4,5,5-tetramethyl-1,3,2-dioxaborolan-2-yl)pyridin-2(1H)-one (i22-2)

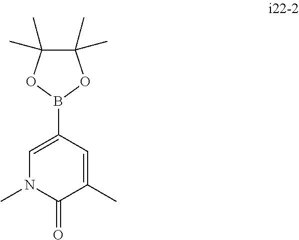

To a solution of 5-bromo-1,3-dimethylpyridin-2-one(1.00 g, 4.949 mmol, 1.00 equiv) and bis(pinacolato)diboron (1508.17 mg, 5.939 mmol, 1.20 equiv) in dioxane (10.00 mL) was added KOAc (971.46 mg, 9.898 mmol, 2.00 equiv) and Pd(dppfCl₂·CH₂Cl₂ (404.18 mg, 0.495 mmol, 0.10 equiv). After stirring for 2 hours at 90° C. under a nitrogen atmosphere, the resulting mixture was concentrated under reduced pressure. The crude product was used in the next step directly without further purification. LCMS (ESI) m/z: [M+H]⁺=250.

### Step 2: Preparation of 4-(1,5-dimethyl-6-oxopyridin-3-yl)-2,6-dimethoxybenzaldehyde (i22-3)

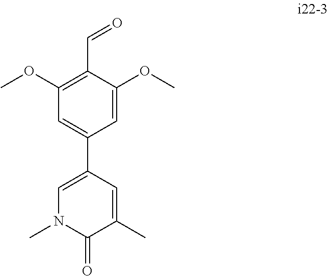

To a solution of 1,3-dimethyl-5-(4,4,5,5-tetramethyl-1,3,2-dioxaborolan-2-yl)pyridin-2-one (1.20 g, 4.817 mmol, 1.00 equiv) and 4-bromo-2,6-dimethoxybenzaldehyde (1.18 g, 4.817 mmol, 1.00 equiv) in 1,4-dioxane (40.00 mL) and H₂O (4.00 mL) was added CS₂CO₃ (3.14 g, 9.634 mmol, 2.00 equiv) and Pd(dppf)Cl₂ (0.35 g, 0.482 mmol, 0.10 equiv). After stirring for 2 hours at 80° C. under a nitrogen atmosphere, the resulting mixture was concentrated under reduced pressure. The residue was purified by silica gel column chromatography, eluted with CH₂Cl₂/MeOH (18:1) to afford 4-(1,5-dimethyl-6-oxopyridin-3-yl)-2,6-dimethoxybenzaldehyde (1.43 g, 87.83%) as a brown syrup. LCMS (ESI) m/z: [M+H]⁺=288.

### Step 3: Preparation of methyl 1-[[4-(1,5-dimethyl-6-oxopyridin-3-yl)-2,6-dimethoxyphenyl]methyl]azetidine-3-carboxylate (i22-4)

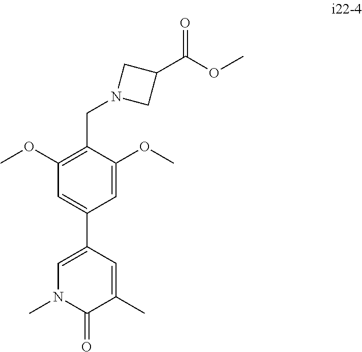

To a solution of methyl azetidine-3-carboxylate hydrochloride (1.13 g, 7.466 mmol, 1.50 equiv) in MeOH (10.00 mL) was added Et₃N to pH 7-8. Then 4-(1,5-dimethyl-6-oxopyridin-3-yl)-2,6-dimethoxybenzaldehyde (1.43 g, 4.977 mmol, 1.00 equiv) was added. After stirring for 5-10 minutes, NaBH₃CN (0.63 g, 9.954 mmol, 2.00 equiv) was added in portions at ambient atmosphere. The resulting mixture was concentrated after stirring for 1 hour at room temperature. The residue was purified by silica gel column chromatography, eluted with CH₂Cl₂/MeOH (20:1) to afford methyl 1-[[4-(1,5-dimethyl-6-oxopyridin-3-yl)-2,6-dimethoxyphenyl]methyl]azetidine-3-carboxylate (1.06 g, 52.36%) as a yellow solid. LCMS (ESI) m/z: [M+H]⁺=387.

### Step 4: Preparation of 1-[[4-(1,5-dimethyl-6-oxopyridin-3-yl)-2,6-dimethoxyphenyl]methyl]azetidine-3-carboxylic acid (i22-5)

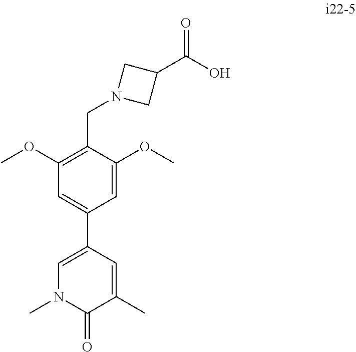 [SMILES: COc1cc(-c2cc(C)c(=O)n(C)c2)cc(OC)c1CN1CC(C(=O)O)C1]

A mixture of methyl 1-[[4-(1,5-dimethyl-6-oxopyridin-3-yl)-2,6-dimethoxyphenyl]methyl]azetidine-3-carboxylate (203.00 mg, 0.525 mmol, 1.00 equiv) in HCl (12 N, 2.00 mL) was stirred for 2 hours at 90° C. The resulting mixture was concentrated under reduced pressure to give 1-[[4-(1,5-dimethyl-6-oxopyridin-3-yl)-2,6-dimethoxyphenyl]methyl]azetidine-3-carboxylic acid (150 mg, 71.31%) as a yellow solid. LCMS (ESI) m/z: [M+H]⁺=373.

### Step 4: Preparation of 2-(2,6-dihydroxypiperidin-3-yl)-4-[(8-[[hydroxy(1-[[4-(6-hydroxy-1,5-dimethyl-1,6-dihydropyridin-3-yl)-2,6-dimethoxyphenyl]methyl]azetidin-3-yl)methyl]amino]octyl)amino]-2,3-dihydro-1H-isoindole-1,3-diol formic acid (compound D16 formic acid)

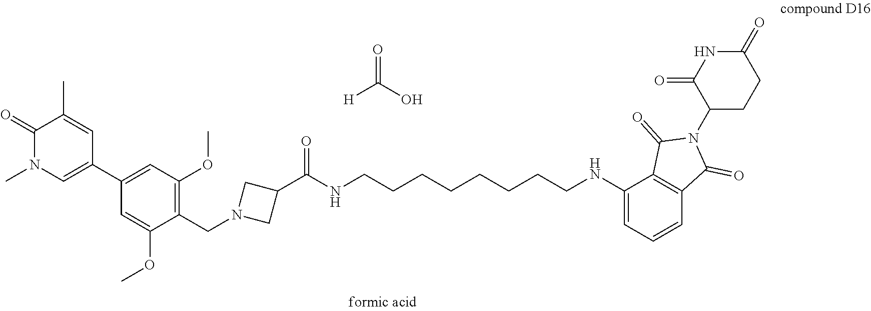 [SMILES: COc1cc(-c2cc(C)c(=O)n(C)c2)cc(OC)c1CN1CC(C(=O)NCCCCCCCCNc2cccc3c2C(=O)N(C2CCC(=O)NC2=O)C3=O)C1 ; O=CO]

To a stirred mixture of 1-[[4-(1,5-dimethyl-6-oxo-1,6-dihydropyridin-3-yl)-2,6-dimethoxyphenyl]methyl]azetidine-3-carboxylic acid trifluoroacetic acid(50 mg, 0.103 mmol, 1 equiv) and 4-[(8-aminooctyl)amino]-2-(2,6-dioxopiperidin-3-yl)-2,3-dihydro-1H-isoindole-1,3-dione hydrochloride (44.91 mg, 0.103 mmol, 1 equiv) in DCM (2 mL) was added DIEA (53.57 mg, 0.415 mmol, 4 equiv). After stirring for 10 minutes, PyBOP (80.89 mg, 0.155 mmol, 1.5 equiv) was added. The resulting mixture was concentrated under reduced pressure, and then the residue was purified by Prep-HPLC (conditions: Sun Fire C18 OBD Prep Column, 19 mm×250 mm; mobile phase, Water (0.1% FA) and ACN (23% Phase B up to 33% in 8 min, hold 33% in 1 minutes); Detector, UV). This resulted in 2-(2,6-dihydroxypiperidin-3-yl)-4-[(8-[[hydroxy(1-[[4-(6-hydroxy-1,5-dimethyl-1,6-dihydropyridin-3-yl)-2,6-dimethoxyphenyl]methyl]azetidin-3-yl)methyl]amino]octyl)amino]-2,3-dihydro-1H-isoindole-1,3-diol formic acid (2.4 mg, 2.73%) as a yellow solid. ¹H NMR (300 MHz, Methanol-d4) δ 8.56 (s, 2H, FA), 7.96 (s, 1H), 7.83 (s, 1H), 7.61-7.50 (m, 1H), 7.04 (d, J=7.7 Hz, 2H), 6.88 (s, 2H), 4.62 (s, 1H), 4.32 (s, 2H), 4.09 (d, J=7.9 Hz, 4H), 3.98 (s, 6H), 3.68 (s, 3H), 3.55-3.44 (m, 2H), 3.21 (t, J=7.0 Hz, 2H), 2.91-2.68 (m, 4H), 2.22 (s, 3H), 2.12 (s, 1H), 1.68 (s, 2H), 1.64-1.39 (m, 10H). LCMS (ESI) m/z: [M+H]⁺=373.17.

### Example 23—Preparation of 3-amino-1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)azetidine-3-carboxamide (compound D17)

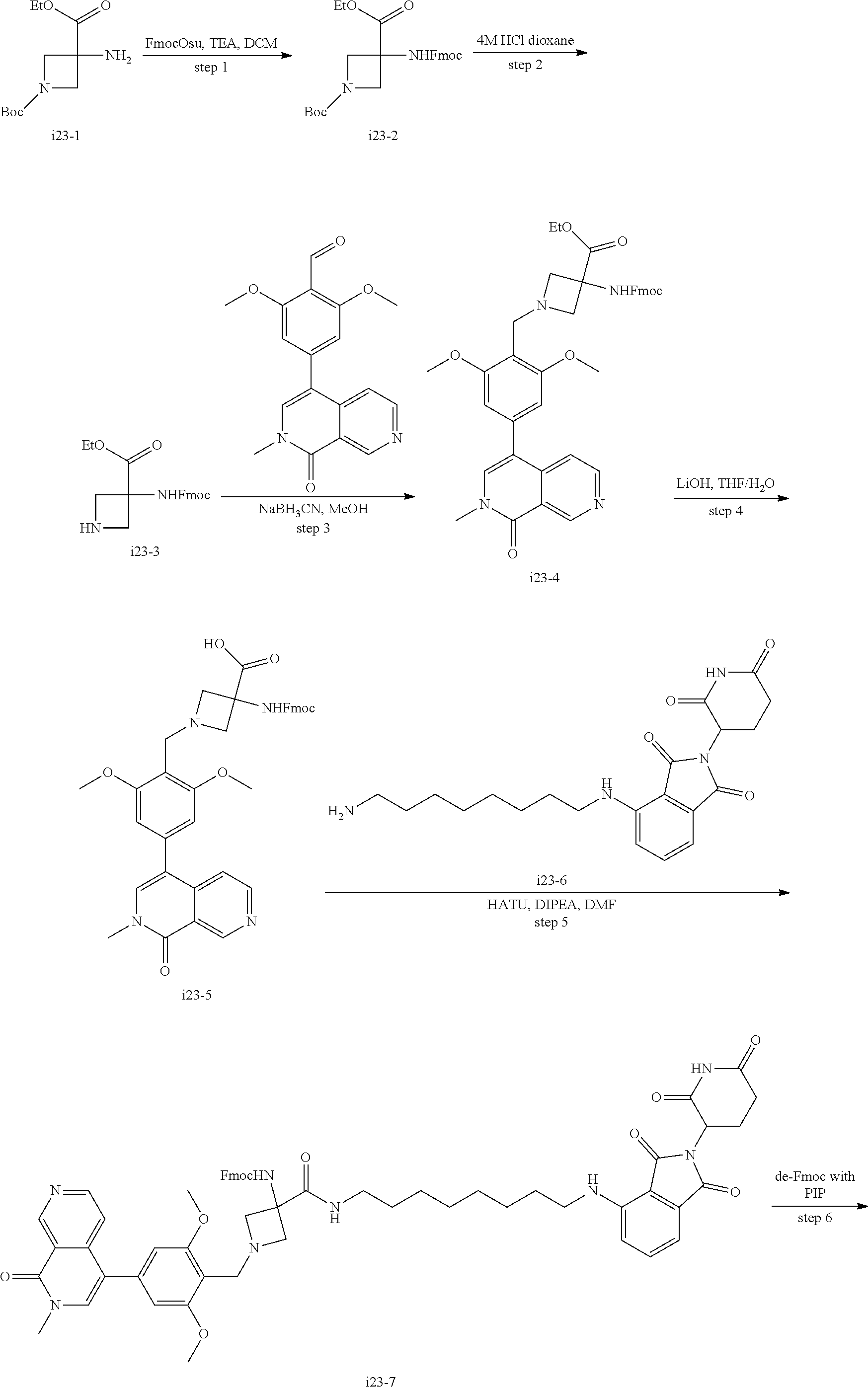

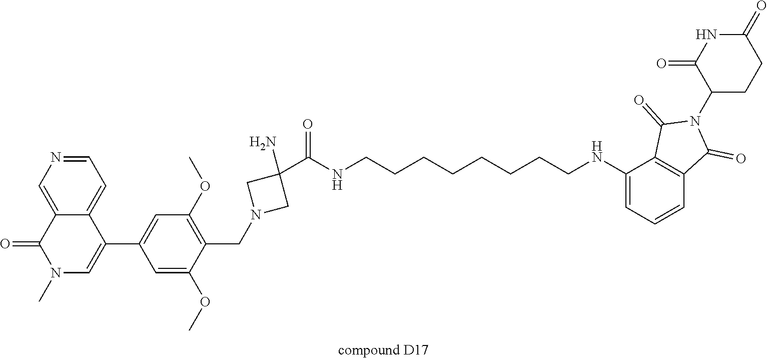 [SMILES: COc1cc(-c2cn(C)c(=O)c3cnccc23)cc(OC)c1CN1CC(N)(C(=O)NCCCCCCCCNc2cccc3c2C(=O)N(C2CCC(=O)NC2=O)C3=O)C1]

### Step 1: Preparation of 1-tert-Butyl 3-ethyl 3-([(9H-fluoren-9-yl)methoxy]carbonyl]amino)azetidine-1,3-dicarboxylate (i23-2)

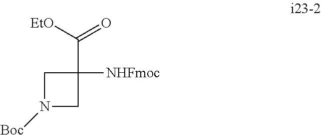

To a solution of 1-tert-butyl 3-ethyl 3-aminoazetidine-1,3-dicarboxylate (120 mg, 0.491 mmol, 1 equiv) and 2,5-dioxopyrrolidin-1-yl (9H-fluoren-9-yl)methyl carbonate (182.3 mg, 0.540 mmol, 1.1 equiv) in DCM (1 mL) was added TEA (149.1 mg, 1.474 mmol, 3 equiv). The resulting solution was stirred at room temperature for 1 hour. The residue was purified by Prep-TLC (PE/EtOAc 1:1) to afford 1-tert-butyl 3-ethyl 3-([[(9H-fluoren-9-yl)methoxy]carbonyl]amino)azetidine-1,3-dicarboxylate(120 mg, 48%) as a white solid. LCMS (ESI) m/z: [M+H]⁺=467.

### Step 2: Preparation of ethyl 3-([[(9H-fluoren-9-yl)methoxy]carbonyl]amino)azetidine-3-carboxylate (i23-3)

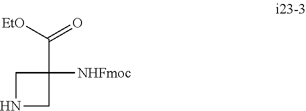

A mixture of 1-tert-butyl 3-ethyl 3-([[(9H-fluoren-9-yl)methoxy]carbonyl]amino)azetidine-1,3-dicarboxylate (120.00 mg, 0.257 mmol, 1.00 equiv) and 4 M HCl in 1,4-dioxane (2 mL) was stirred at room temperature for 1 hour. The reaction solution was concentrated under reduced pressure to afford ethyl 3-([[(9H-fluoren-9-yl)methoxy]carbonyl]amino)azetidine-3-carboxylate (120 mg, 89%) as a white solid that was used directly without further purification. LCMS (ESI) m/z: [M+H]⁺=367.

### Step 3: Preparation of Ethyl 1-[[2,6-Dimethoxy-4-(2-methyl-1-oxo-1,2-dihydro-2,7-naphthyridin-4-yl)phenyl]methyl]-3-([[(9H-fluoren-9-yl)methoxy]carbonyl]amino)azetidine-3-carboxylate (i23-4)

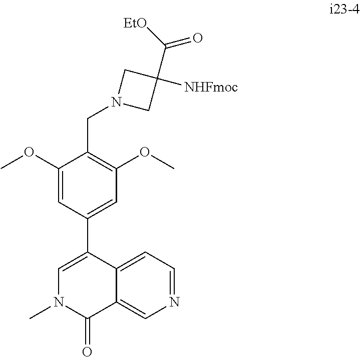

To a solution of 2,6-dimethoxy-4-(2-methyl-1-oxo-1,2-dihydro-2,7-naphthyridin-4-yl)benzaldehyde (127.5 mg, 0.393 mmol, 1.20 equiv) and ethyl 3-([[(9H-fluoren-9-yl)methoxy]carbonyl]amino)azetidine-3-carboxylate (120 mg, 0.327 mmol, 1 equiv) in MeOH (1 mL) was added NaBH₃CN (41.2 mg, 0.655 mmol, 2 equiv). The resulting solution was stirred at room temperature for 1 hour. The mixture was then concentrated under reduced pressure and the residue was purified by Prep-TLC (CH₂Cl₂/MeOH 12:1) to afford ethyl 1-[[2,6-dimethoxy-4-(2-methyl-1-oxo-1,2-dihydro-2,7-naphthyridin-4-yl)phenyl]methyl]-3-([[(9H-fluoren-9-yl)methoxy]carbonyl]amino)azetidine-3-carboxylate (100 mg, 45%) as a yellow solid. LCMS (ESI) m/z: [M+H]+=675.

### Step 4: Preparation of Ethyl 1-[[2,6-dimethoxy-4-(2-methyl-1-oxo-1,2-dihydro-2,7-naphthyridin-4-yl)phenyl]methyl]-3-([[(9H-fluoren-9-yl)methoxy]carbonyl]amino)azetidine-3-carboxylic acid (i23-5)

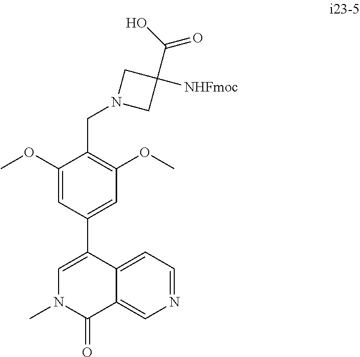

A solution of ethyl 1-[[2,6-dimethoxy-4-(2-methyl-1-oxo-1,2-dihydro-2,7-naphthyridin-4-yl)phenyl]methyl]-3-([[(9H-fluoren-9-yl)methoxy]carbonyl]amino)azetidine-3-carboxylate (100 mg, 0.148 mmol, 1 equiv) in concentrated HCl (2 mL) was stirred at 90° C. for 1 hour. The resulting mixture was concentrated under reduced pressure to afford 1-[[2,6-dimethoxy-4-(2-methyl-1-oxo-1,2-dihydro-2,7-naphthyridin-4-yl)phenyl]methyl]-3-([[(9H-fluoren-9-yl)methoxy]carbonyl]amino)azetidine-3-carboxylic acid (100 mg, 94%) as a yellow solid that was used directly without further purification. LCMS (ESI) m/z: [M+H]+=647.3

### Step 5: Preparation of (9H-fluoren-9-yl)methyl N-(1-[[2,6-dimethoxy-4-(2-methyl-1-oxo-1,2-dihydro-2,7-naphthyridin-4-yl)phenyl]methyl]-3-[(8-[[2-(2,6-dioxopiperidin-3-yl)-1, 3-dioxo-2,3-dihydro-1H-isoindol-4-yl]amino]octyl)carbamoyl]azetidin-3-yl)carbamate (i23-7)

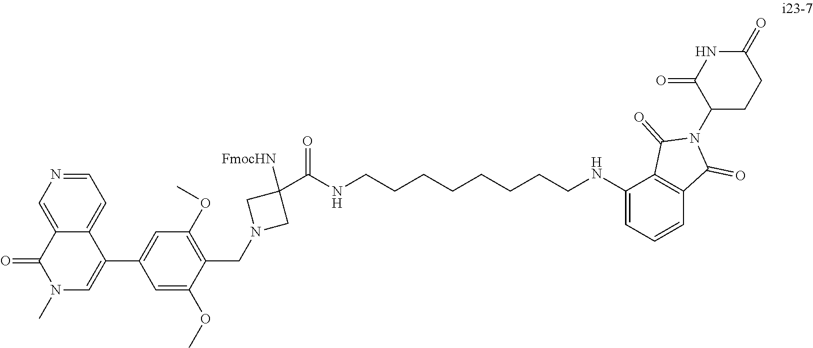

To a solution of 1-[[2,6-dimethoxy-4-(2-methyl-1-oxo-1,2-dihydro-2,7-naphthyridin-4-yl)phenyl]methyl]-3-([[(9H-fluoren-9-yl)methoxy]carbonyl]amino)azetidine-3-carboxylic acid (100 mg, 0.155 mmol, 1 equiv) and 4-[(8-aminooctyl)amino]-2-(2,6-dioxopiperidin-3-yl)-2,3-dihydro-1H-isoindole-1,3-dione (74.3 mg, 0.186 mmol, 1.2 equiv) in DMF (1 mL) was added DIEA (60.0 mg, 0.464 mmol, 3 equiv) and HATU (88.2 mg, 0.232 mmol, 1.5 equiv). The resulting solution was stirred at room temperature for 1 hour. The mixture was then concentrated under reduced pressure and the residue was purified by Prep-TLC (CH₂Cl₂/MeOH 12:1) to afford (9H-fluoren-9-yl)methyl N-(1-[[2,6-dimethoxy-4-(2-methyl-1-oxo-1,2-dihydro-2,7-naphthyridin-4-yl)phenyl]methyl]-3-[(8-[[2-(2,6-dioxopiperidin-3-yl)-1,3-dioxo-2,3-dihydro-1H-isoindol-4-yl]amino]octyl)carbamoyl]azetidin-3-yl)carbamate (90 mg, 51%) as a yellow solid. LCMS (ESI) m/z: [M+H]+=1029.

### Step 6: Preparation of 3-Amino-1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)azetidine-3-carboxamide (compound D17)

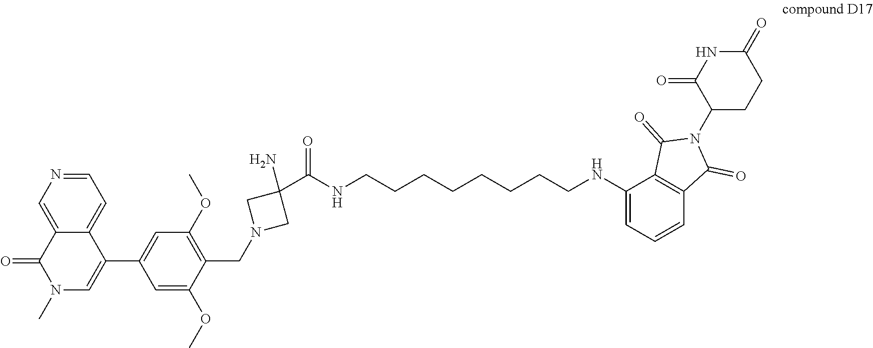 [SMILES: COc1cc(-c2cn(C)c(=O)c3cnccc23)cc(OC)c1CN1CC(N)(C(=O)NCCCCCCCCNc2cccc3c2C(=O)N(C2CCC(=O)NC2=O)C3=O)C1]

A solution of (9H-fluoren-9-yl)methyl N-(1-[[2,6-dimethoxy-4-(2-methyl-1-oxo-1,2-dihydro-2,7-naphthyridin-4-yl)phenyl]methyl]-3-[(8-[[2-(2,6-dioxopiperidin-3-yl)-1,3-dioxo-2,3-dihydro-1H-isoindol-4-yl]amino]octyl)carbamoyl]azetidin-3-yl)carbamate (90 mg, 0.087 mmol, 1.00 equiv) in piperidine (1 mL) and DMF (4 mL) was stirred at room temperature for 1 hour. The crude solution was purified by Prep-HPLC (condition: SunFire C18 OBD Prep Column, 100 A, 5 μm, 19 mm×250 mm; Mobile Phase A: Water (0.1% FA), Mobile Phase B: ACN; Flow rate: 25 mL/minuteute; Gradient: 28% B to 28% B in 2 minutes; 254 nm; Rₜ: 6.9 minutes) to afford 3-amino-1-[[2,6-dimethoxy-4-(2-methyl-1-oxo-1,2-dihydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)azetidine-3-carboxamide (3.8 mg, 5.2%) as a yellow solid. ¹H NMR (300 MHz, Acetonitrile-d3) 6 9.52 (s, 1H), 8.70 (d, J=5.7 Hz, 1H), 8.26 (s, 0.53H, FA), 7.78-7.42 (m, 4H), 7.02 (dd, J=7.8, 4.2 Hz, 2H), 6.75 (s, 2H), 6.30 (t, J=5.9 Hz, 1H), 4.95 (dd, J=12.4, 5.2 Hz, 1H), 4.10 (s, 2H), 3.95 (d, J=8.8 Hz, 2H), 3.87 (s, 6H), 3.50 (s, 3H), 3.24 (dq, J=23.4, 6.6 Hz, 4H), 2.83-2.59 (m, 3H), 1.63 (s, 2H), 1.49 (s, 2H), 1.32 (d, J=13.1 Hz, 10H). LCMS (ESI) m/z: [M+H]+=807.40.

### Example 24—Preparation of (2S)-1-[[2,6-dimethoxy-4-(2-methyl-1-oxo-2,7-naphthyridin-4-yl)phenyl]methyl]-N-(8-[[2-(2,6-dioxopiperidin-3-yl)-1,3-dioxoisoindol-4-yl]amino]octyl)azetidine-2-carboxamide (compound D18)

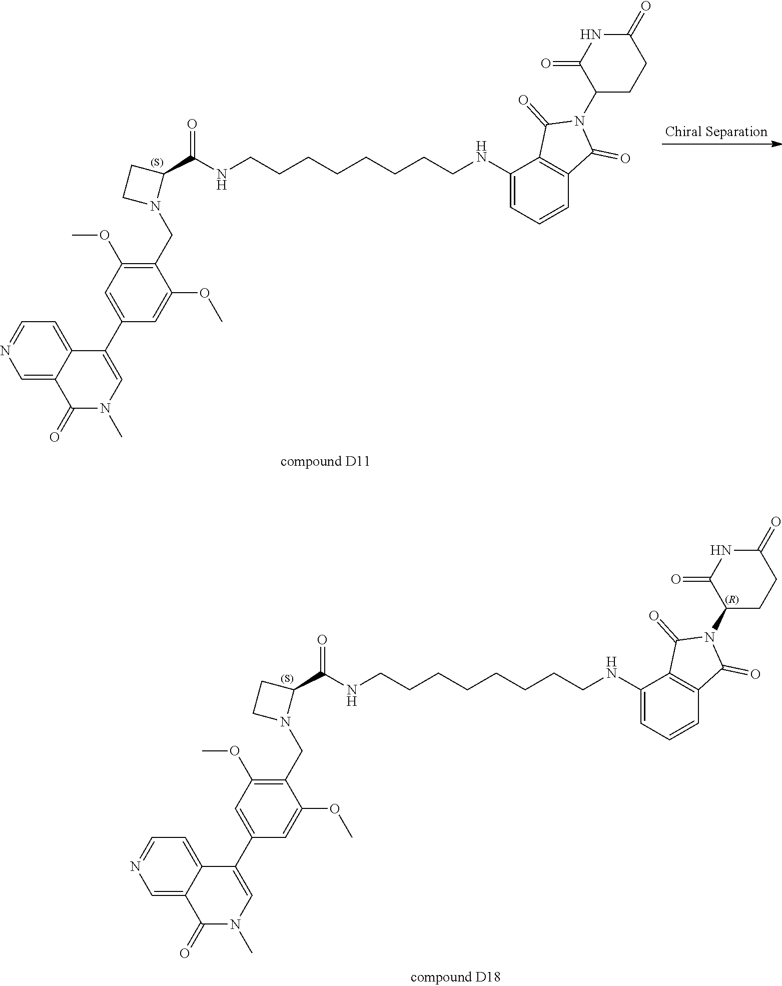

Compound D11 was further separated by chiral HPLC to afford (2S)-1-((2,6-dimethoxy-4-(2-methyl-1-oxo-2,7-naphthyridin-4-yl)phenyl)methyl)-N-(8-((2-((R)-2,6-dioxopiperidin-3-yl)-1,3-dioxoisoindol-4-yl)amino)octyl) azetidine-2-carboxamide (10 mg, 10.34%) as a yellow solid. ¹H NMR (400 MHz, Methanol-d4) δ 9.51 (s, 1H), 8.68 (d, J=5.7 Hz, 1H), 7.72 (s, 1H), 7.62 (d, J=5.8 Hz, 1H), 7.53 (t, J=7.8 Hz, 1H), 7.00 (dd, J=10.6, 7.8 Hz, 2H), 6.75 (s, 2H), 5.05 (dd, J=12.4, 5.4 Hz, 1H), 3.89 (s, 9H), 3.69 (s, 3H), 3.30 (s, 2H), 3.25 (t, J=6.9 Hz, 2H), 3.15 (t, J=7.1 Hz, 2H), 2.94-2.64 (m, 3H), 2.35 (d, J=9.5 Hz, 1H), 2.16-2.00 (m, 1H), 1.58 (t, J=7.1 Hz, 2H), 1.40 (d, J=6.7 Hz, 2H), 1.30 (s, 8H). LCMS (ESI) m/z: [M+H]+=792.60.

### Example 25—Preparation of (2S)-1-[[2,6-dimethoxy-4-(2-methyl-1-oxo-2,7-naphthyridin-4-yl)phenyl]methyl]-N-(8-[[2-(2,6-dioxopiperidin-3-yl)-1,3-dioxoisoindol-4-yl]amino]octyl)azetidine-2-carboxamide (compound D19)

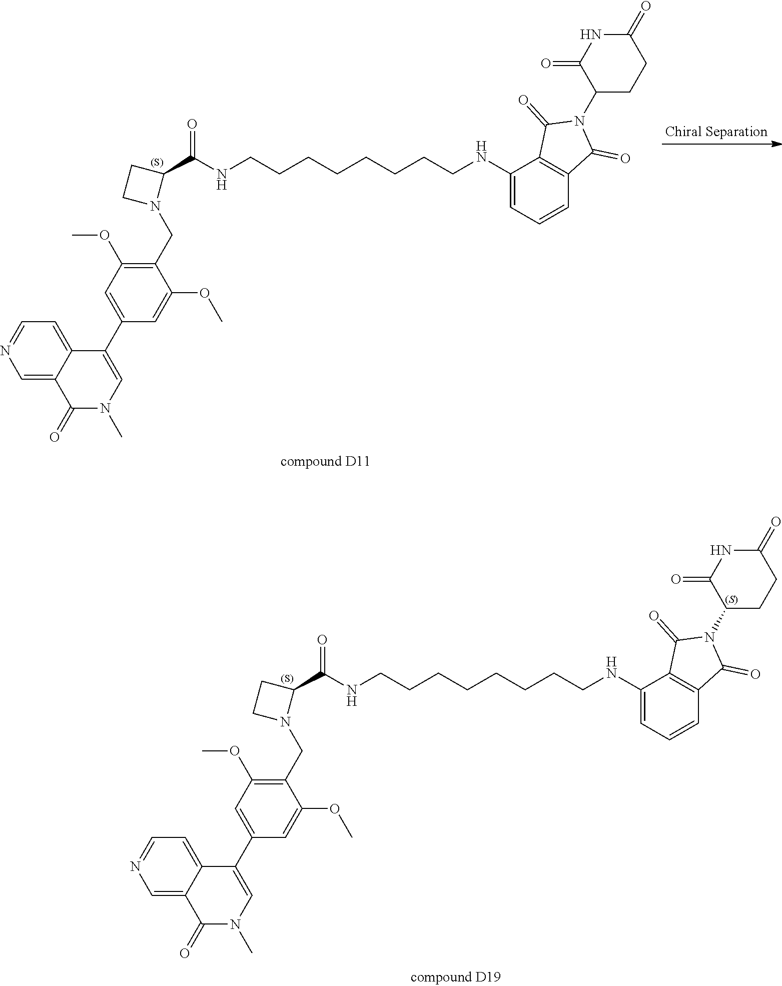

Compound D11 was further separated by chiral HPLC to afford (2S)-1-((2,6-dimethoxy-4-(2-methyl-1-oxo-2,7-naphthyridin-4-yl)phenyl)methyl)-N-(8-((2-((S)-2,6-dioxopiperidin-3-yl)-1,3-dioxoisoindol-4-yl)amino)octyl) azetidine-2-carboxamide (10 mg, 10.34%) as a yellow solid. ¹H NMR (400 MHz, Methanol-d4) δ 9.51 (s, 1H), 8.68 (d, J=5.7 Hz, 1H), 7.72 (s, 1H), 7.62 (d, J=5.8 Hz, 1H), 7.53 (t, J=7.8 Hz, 1H), 7.00 (dd, J=10.6, 7.8 Hz, 2H), 6.75 (s, 2H), 5.05 (dd, J=12.4, 5.4 Hz, 1H), 3.89 (s, 9H), 3.69 (s, 3H), 3.30 (s, 2H), 3.25 (t, J=6.9 Hz, 2H), 3.15 (t, J=7.1 Hz, 2H), 2.94-2.64 (m, 3H), 2.35 (d, J=9.5 Hz, 1H), 2.16-2.00 (m, 1H), 1.58 (t, J=7.1 Hz, 2H), 1.40 (d, J=6.7 Hz, 2H), 1.30 (s, 8H). LCMS (ESI) m/z: [M+H]+=792.60

### Example 26—Preparation of 6-[[2,6-dimethoxy-4-(2-methyl-1-oxo-1,2,4a,8a-tetrahydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)spiro[3.3]heptane-2-carboxamide (compound D20)

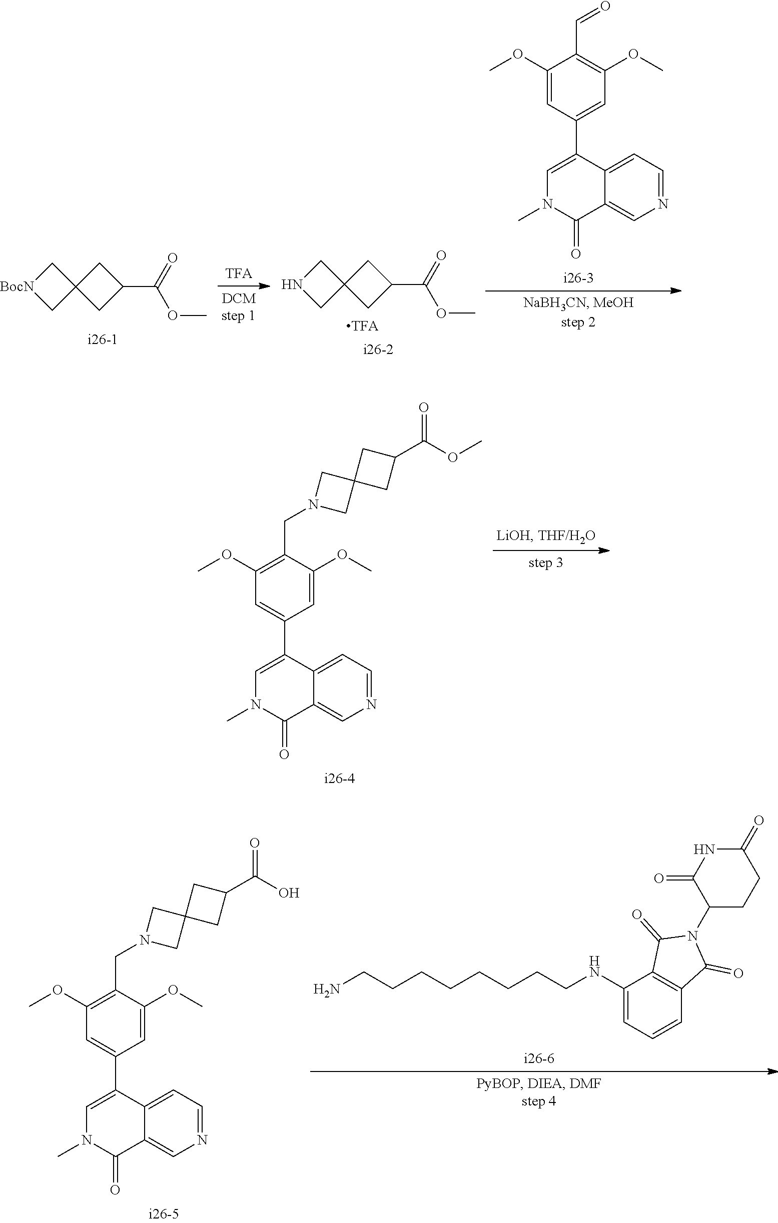

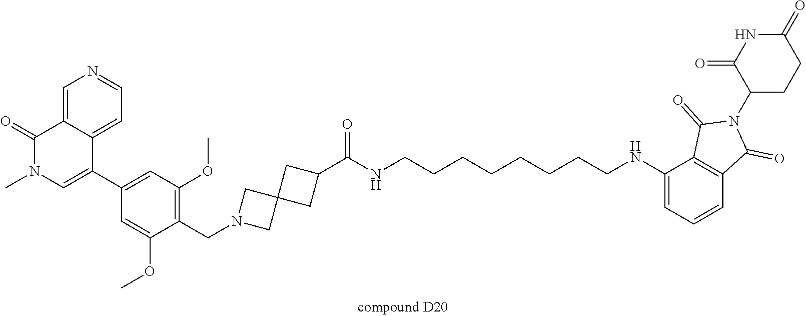 [SMILES: COc1cc(-c2cn(C)c(=O)c3cnccc23)cc(OC)c1CN1CC2(CC(C(=O)NCCCCCCCCNc3cccc4c3C(=O)N(C3CCC(=O)NC3=O)C4=O)C2)C1]

### Step 1: Preparation of methyl 2-azaspiro[3.3]heptane-6-carboxylate trifluoroacetic acid (i26-2)

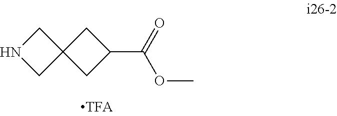

A mixture of 2-tert-butyl 6-methyl 2-azaspiro[3.3]heptane-2,6-dicarboxylate (127.60 mg, 0.500 mmol, 1.00 equiv) and TFA (1 mL) in DCM (3.00 mL) was stirred for 2 hours at room temperature. Then, the solvent was evaporated, and the resulting residue was used in the next step directly without further purification. LCMS (ESI) m/z: [M+H]+=156.

### Step 2: Preparation of methyl 2-[[2,6-dimethoxy-4-(2-methyl-1-oxo-2,7-naphthyridin-4-yl)phenyl]methyl]-2-azaspiro[3.3]heptane-6-carboxylate (i26-4)

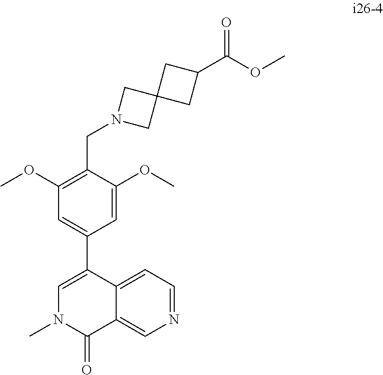

To a stirred solution of methyl 2-azaspiro[3.3]heptane-6-carboxylate trifluoroacetic acid (77.60 mg, 0.288 mmol, 1.00 equiv), Et₃N (116.67 mg, 1.153 mmol, 4 equiv), and 2,6-dimethoxy-4-(2-methyl-1-oxo-2,7-naphthyridin-4-yl)benzaldehyde (93.49 mg, 0.288 mmol, 1 equiv) in MeOH (2.00 mL) was added NaBH₃CN (36.23 mg, 0.576 mmol, 2 equiv) in portions at room temperature. After the solvent was evaporated, the residue was purified by silica gel column chromatography, eluted with CH₂Cl₂/MeOH (12:1) to afford methyl 2-[[2,6-dimethoxy-4-(2-methyl-1-oxo-2,7-naphthyridin-4-yl)phenyl]methyl]-2-azaspiro[3.3]heptane-6-carboxylate (156 mg, 96.91%) as a yellow solid. LCMS (ESI) m/z: [M+H]+=464.

### Step 3: Preparation of 2-[[2,6-dimethoxy-4-(2-methyl-1-oxo-2,7-naphthyridin-4-yl)phenyl]methyl]-2-azaspiro[3.3]heptane-6-carboxylic acid (i26-5)

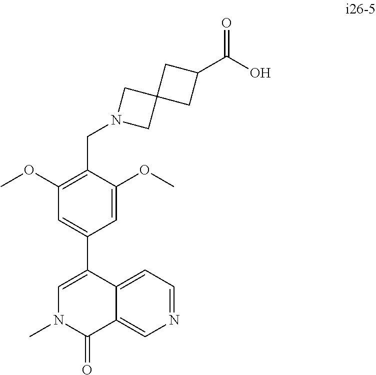 [SMILES: COc1cc(-c2cn(C)c(=O)c3cnccc23)cc(OC)c1CN1CC2(CC(C(=O)O)C2)C1]

A solution of methyl 2-[[2,6-dimethoxy-4-(2-methyl-1-oxo-2,7-naphthyridin-4-yl)phenyl]methyl]-2-azaspiro[3.3]heptane-6-carboxylate (156.00 mg, 0.347 mmol, 1.00 equiv) and LiOH (83.28 mg, 3.47 mmol, 10.0 equiv) in mixed THE (2.00 mL) and H₂O (1.00 mL) was stirred for 1 hour at room temperature. Then solvent was evaporated, and the resulting solution was purified by Prep-HPLC (0-100% ACN/water, with 0.1% TFA) to afford 2-[[2,6-dimethoxy-4-(2-methyl-1-oxo-2,7-naphthyridin-4-yl)phenyl]methyl]-2-azaspiro[3.3]heptane-6-carboxylic acid(114.7 mg, 75.89%) as a dark yellow oil. LCMS (ESI) m/z: [M+H]+=450.

### Step 4: Preparation of 6-[[2,6-dimethoxy-4-(2-methyl-1-oxo-1,2,4a,8a-tetrahydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)spiro[3.3]heptane-2-carboxamide (compound D20)

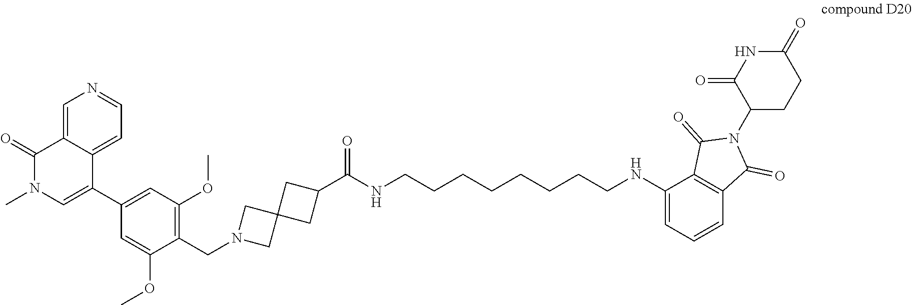 [SMILES: COc1cc(-c2cn(C)c(=O)c3cnccc23)cc(OC)c1CN1CC2(CC(C(=O)NCCCCCCCCNc3cccc4c3C(=O)N(C3CCC(=O)NC3=O)C4=O)C2)C1]

To a stirred solution of 6-[[2,6-dimethoxy-4-(2-methyl-1-oxo-1,2,4a,8a-tetrahydro-2,7-naphthyridin-4-yl)phenyl]methyl]spiro[3.3]heptane-2-carboxylic acid (45 mg, 0.100 mmol, 1 equiv) and 4-[(8-aminooctyl)amino]-2-(2,6-dioxopiperidin-3-yl)-2,3-dihydro-1H-isoindole-1,3-dione (40.00 mg, 0.100 mmol, 1 equiv) in DMF (0.5 mL), was added DIEA (64.54 mg, 0.499 mmol, 5 equiv) and PyBOP (103.95 mg, 0.200 mmol, 2 equiv) at room temperature. The mixture was stirred for 1 h and directly purified by Prep-HPLC with the following conditions (conditions: SunFire C18 OBD Prep Column, 19 mm×250 mm; Mobile Phase A: Water (0.1% FA), Mobile Phase B: ACN; Flow rate: 25 mL/minuteute; Gradient: 29% B to 32% B in 8 minutes; 254 nm; Rₜ: 6.55 minutes) to afford 6-[[2,6-dimethoxy-4-(2-methyl-1-oxo-1,2,4a,8a-tetrahydro-2,7-naphthyridin-4-yl)phenyl]methyl]-N-(8-[[2-(2,6-dioxopiperidin-3-yl)-1,3-dioxo-2,3-dihydro-1H-isoindol-4-yl]amino]octyl)spiro[3.3]heptane-2-carboxamide (14.1 mg, 14.24%) as a yellow solid. ¹H NMR (300 MHz, Methanol-d4) δ 9.54 (d, J=0.9 Hz, 1H), 8.69 (d, J=5.8 Hz, 1H), 7.78 (s, 1H), 7.65-7.50 (m, 2H), 7.04 (d, J=7.9 Hz, 2H), 6.86 (s, 2H), 5.05 (dd, J=12.6, 5.7 Hz, 1H), 4.63 (s, 2H), 4.44 (s, 2H), 4.18 (s, 3H), 3.97 (s, 6H), 3.88 (s, 1H), 3.71 (s, 3H), 3.34-3.11 (m, 3H), 3.10-2.67 (m, 5H), 2.61-2.37 (m, 4H), 2.27-2.13 (m, 1H), 1.67 (q, J=7.0 Hz, 2H), 1.59-1.26 (m, 10H). LCMS (ESI) m/z: [M+H]+=832.5.

### Example 27—Preparation of 6-[[2,6-dimethoxy-4-(2-methyl-1-oxo-1,2,4a,8a-tetrahydro-2,7-naphthyridin-4-yl)phenyl]methyl]-N-(6-[[2-(2,6-dioxopiperidin-3-yl)-1,3-dioxo-2,3-dihydro-1H-isoindol-4-yl]amino]hexyl)spiro[3.3]heptane-2-carboxamide (compound D21)

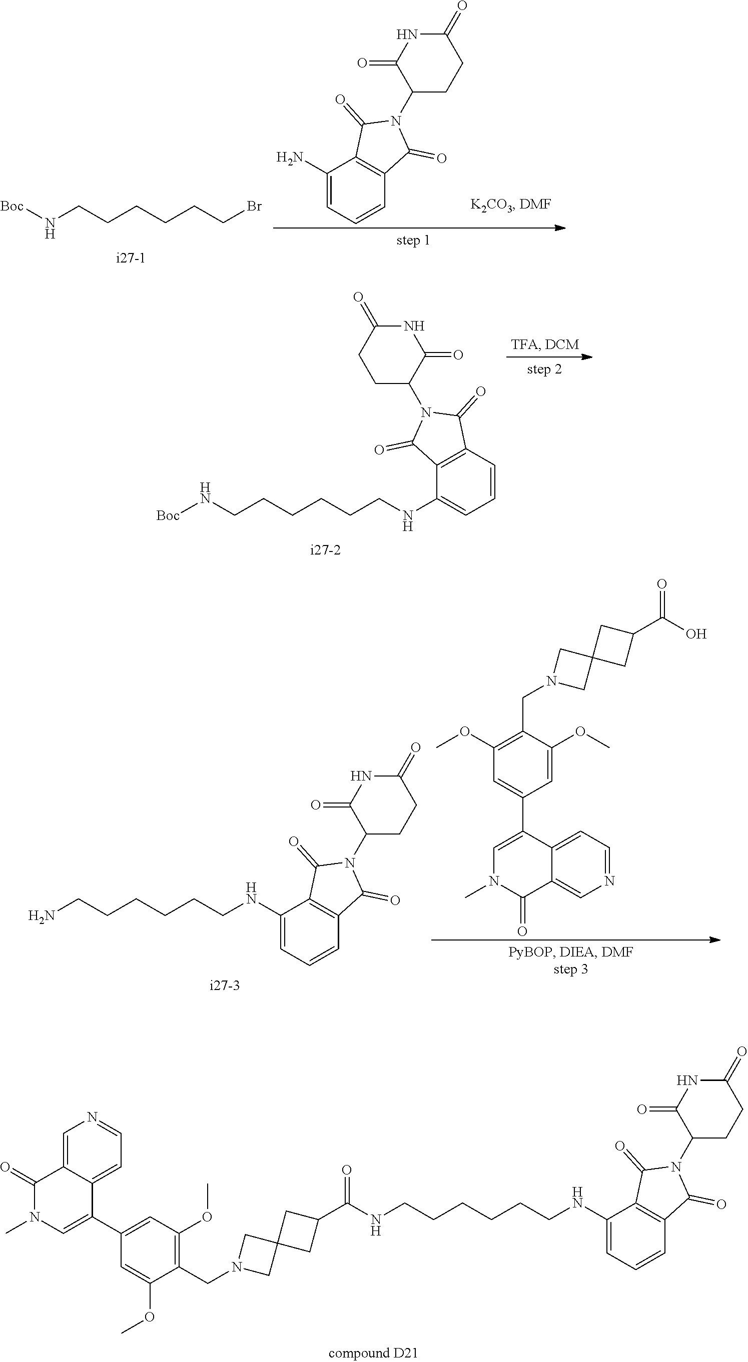

### Step 1: preparation o tert-butyl N-(6-[[2-(2,6-dioxopiperidin-3-yl)-1,3-dioxoisoindol-4-yl]amino]hexyl) carbamate (i27-2)

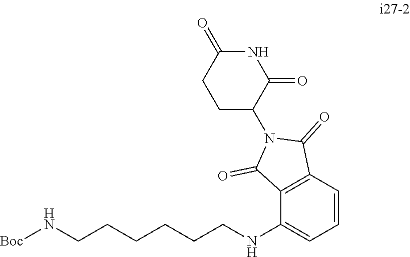 [SMILES: CC(C)(C)OC(=O)NCCCCCCNc1cccc2c1C(=O)N(C1CCC(=O)NC1=O)C2=O]

To a stirred solution of pomalidomide (150.30 mg, 0.550 mmol, 1.00 equiv) and tert-butyl N-(6-bromohexyl)carbamate (i54.13 mg, 0.550 mmol, 1 equiv) in DMF (1.00 mL) was added K₂CO₃ (152.04 mg, 1.100 mmol, 2 equiv) at room temperature. The resulting mixture was stirred overnight at room temperature, and then it was concentrated and purified by silica gel column chromatography, elutinged with PE/EtOAc (10:1) to afford tert-butyl N-(6-[[2-(2,6-dioxopiperidin-3-yl)-1,3-dioxoisoindol-4-yl]amino]hexyl) carbamate (293 mg, 95.82%) as a yellow oil. LCMS (ESI) m/z: [M+H]+=473.

### Step 2: Preparation of 4-[(6-aminohexyl)amino]-2-(2,6-dioxopiperidin-3-yl)isoindole-1,3-dione trifluoroacetic acid (i27-3)

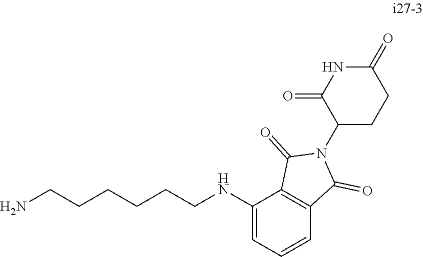 [SMILES: NCCCCCCNc1cccc2c1C(=O)N(C1CCC(=O)NC1=O)C2=O]

A solution of tert-butyl N-(6-[[2-(2,6-dioxopiperidin-3-yl)-1,3-dioxoisoindol-4-yl]amino]hexyl) carbamate (293.00 mg, 0.620 mmol, 1.00 equiv) and TFA (2.0 mL) in DCM (5.00 mL) was stirred for 1 h at room temperature. The mixture was then concentrated to afford 4-[(6-aminohexyl)amino]-2-(2,6-dioxopiperidin-3-yl)isoindole-1,3-dione (243 mg, 80.56%) as a yellow semi-solid, that was used directly without further purification. LCMS (ESI) m/z: [M+H]+=373.

### Step 3: Preparation of 6-[[2,6-dimethoxy-4-(2-methyl-1-oxo-1,2,4a,8a-tetrahydro-2,7-naphthyridin-4-yl)phenyl]methyl]-N-(6-[[2-(2,6-dioxopiperidin-3-yl)-1,3-dioxo-2,3-dihydro-1H-isoindol-4-yl]amino]hexyl) spiro[3.3]heptane-2-carboxamide (compound D21)

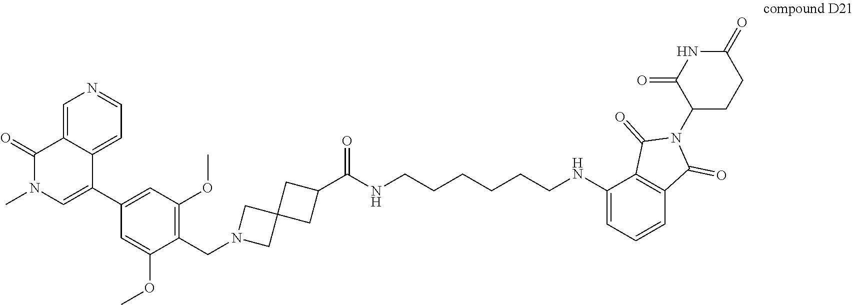 [SMILES: COc1cc(-c2cn(C)c(=O)c3cnccc23)cc(OC)c1CN1CC2(CC(C(=O)NCCCCCCNc3cccc4c3C(=O)N(C3CCC(=O)NC3=O)C4=O)C2)C1]

To a stirred solution of 6-[[2,6-dimethoxy-4-(2-methyl-1-oxo-1,2,4a,8a-tetrahydro-2,7-naphthyridin-4-yl)phenyl]methyl]spiro[3.3]heptane-2-carboxylic acid (30 mg, 0.067 mmol, 1 equiv) in DMF (0.5 mL) was added DIEA (43.03 mg, 0.333 mmol, 5 equiv), PyBOP (69.30 mg, 0.133 mmol, 2 equiv), and 4-[(6-aminohexyl)amino]-2-(2,6-dioxopiperidin-3-yl)-2,3-dihydro-1H-isoindole-1,3-dione(24.80 mg, 0.067 mmol, 1 equiv). The reaction was stirred at ambient atmosphere for 1 hour. The mixture was purified directly by Prep-HPLC (condition: SunFire C18 OBD Prep Column, 100 A, 5 μm, 19 mm×250 mm; Mobile Phase A: Water (0.1% FA), Mobile Phase B: ACN; Flow rate: 25 mL/minuteute; Gradient: 12% B to 38% B in 8 minutes; 254 nm; Rₜ: 7.58 minutes), to afford 6-[[2,6-dimethoxy-4-(2-methyl-1-oxo-1,2,4a,8a-tetrahydro-2,7-naphthyridin-4-yl)phenyl]methyl]-N-(6-[[2-(2,6-dioxopiperidin-3-yl)-1,3-dioxo-2,3-dihydro-1H-isoindol-4-yl]amino]hexyl)spiro[3.3]heptane-2-carboxamide(11.2 mg, 20.90%) as a yellow solid. ¹H NMR (400 MHz, Methanol-d4) δ 9.52 (s, 1H), 8.69 (d, J=5.8 Hz, 1H), 7.77 (s, 1H), 7.61 (d, J=5.8 Hz, 1H), 7.50-7.39 (m, 1H), 7.01 (dd, J=17.7, 7.7 Hz, 2H), 6.85 (s, 2H), 5.09 (dd, J=12.9, 5.5 Hz, 1H), 4.42 (s, 2H), 4.16 (d, J=3.1 Hz, 4H), 3.96 (s, 6H), 3.78 (t, J=7.4 Hz, 2H), 3.71 (s, 3H), 3.50 (q, J=7.3 Hz, 1H), 3.20 (qd, J=7.3, 5.4 Hz, 9H), 2.99-2.87 (m, 2H), 2.91-2.83 (m, 1H), 2.75-2.61 (m, 1H), 2.53 (s, 2H), 2.53-2.47 (m, 1H), 2.47-2.37 (m, 2H), 2.22-2.09 (m, 2H), 1.94 (s, 2H), 1.93 (s, 6H), 1.61 (s, 1H), 1.51 (tt, J=15.1, 8.0 Hz, 4H), 1.46-1.26 (m, 23H), 1.12 (t, J=7.3 Hz, 10H), 0.91 (q, J=9.7, 7.9 Hz, 3H). LCMS (ESI) m/z: [M+H]+=804.40.

### Example 28—Preparation of 1-[[2,6-dimethoxy-4-(2-methyl-1-oxo-1,2-dihydro-2,7-naphthyridin-4-yl)phenyl]methyl]-N-(4-[[2-(2,6-dioxopiperidin-3-yl)-1,3-dioxo-2,3-dihydro-1H-isoindol-5-yl]amino]butyl) azetidine-3-sulfonamide formic acid (compound D22 formic acid)

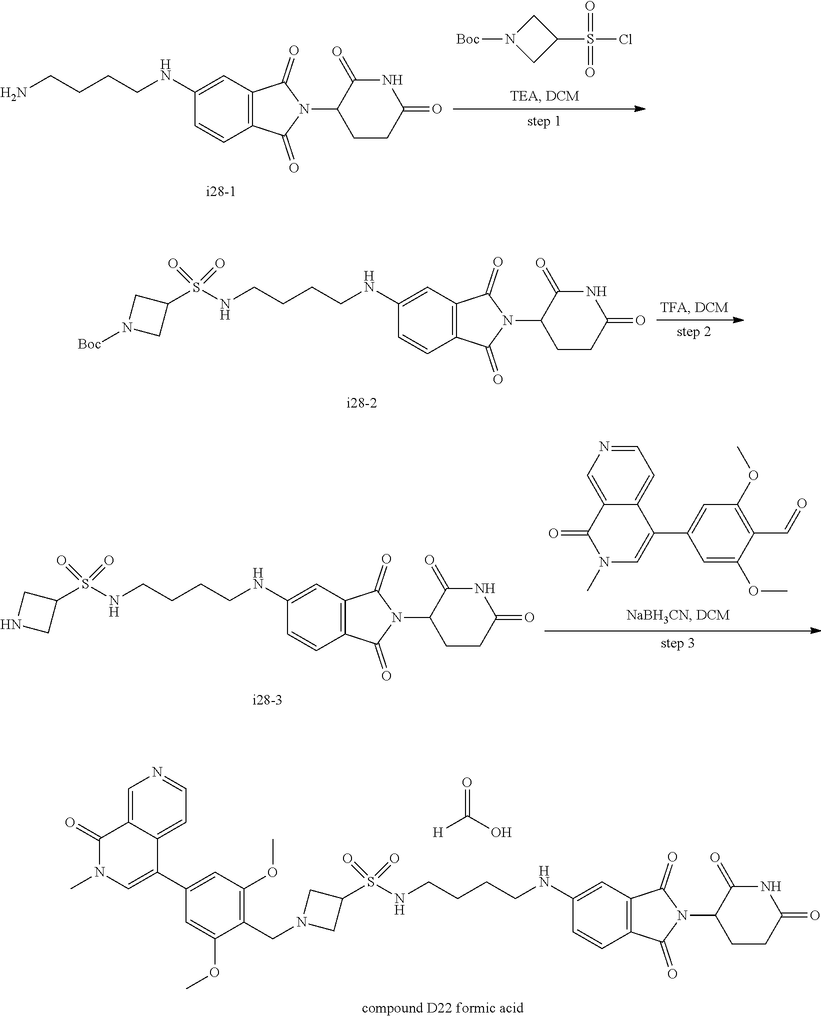

### Step 1: Preparation of tert-butyl-3-[(4-[[2-(2,6-dioxopiperidin-3-yl)-1,3-dioxoisoindol-5-yl]amino]butyl)sulfamoyl]azetidine-1-carboxylate (i28-2)

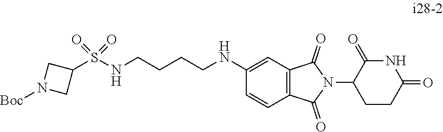 [SMILES: CC(C)(C)OC(=O)N1CC(S(=O)(=O)NCCCCNc2ccc3c(c2)C(=O)N(C2CCC(=O)NC2=O)C3=O)C1]

To a stirred mixture of 5-[(4-aminobutyl)amino]-2-(2,6-dioxopiperidin-3-yl)isoindole-1,3-dione (60.00 mg, 0.174 mmol, 1.00 equiv) and tert-butyl 3-(chlorosulfonyl)azetidine-1-carboxylate (111.38 mg, 0.436 mmol, 2.50 equiv) in DCM (2.00 mL) was added TEA (52.89 mg, 0.523 mmol, 3.00 equiv). After stirring for 1.5 hours at room temperature, the resulting mixture was concentrated under reduced pressure. The residue was purified by Prep-TLC (CH₂Cl₂/EtOAc (1:2)) to afford tert-butyl-3-[(4-[[2-(2,6-dioxopiperidin-3-yl)-1,3-dioxoisoindol-5-yl]amino]butyl)sulfamoyl]azetidine-1-carboxylate(78 mg, 73.87%) as a yellow solid. LCMS (ESI) m/z: [M+H]⁺=564.20.

### Step 2: Preparation of N-(4-((2-(2,6-dioxopiperidin-3-yl)-1,3-dioxoisoindolin-5-yl)amino)butyl)azetidine-3-sulfonamide (i28-3)

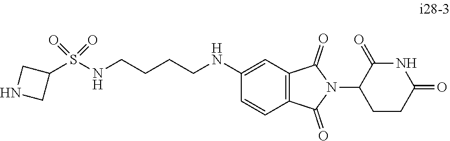 [SMILES: O=C1CCC(N2C(=O)c3ccc(NCCCCNS(=O)(=O)C4CNC4)cc3C2=O)C(=O)N1]

To a stirred mixture of tert-butyl-3-[(4-[[2-(2,6-dioxopiperidin-3-yl)-1,3-dioxoisoindol-5-yl]amino]butyl)sulfamoyl]azetidine-1-carboxylate (78.00 mg, 0.138 mmol, 1.00 equiv) in DCM (2.00 mL, 0.012 mmol, 0.10 equiv) was added TFA (0.40 mL, 5.385 mmol, 38.91 equiv). After stirring for 1 hour at room temperature, the resulting mixture was concentrated under reduced pressure. The residue was used in the next step directly without further purification. LCMS (ESI) m/z: [M+H]⁺=464.15.

### Step 3: Preparation of 1-[[2,6-dimethoxy-4-(2-methyl-1-oxo-1,2-dihydro-2,7-naphthyridin-4-yl)phenyl]methyl]-N-(4-[[2-(2,6-dioxopiperidin-3-yl)-1,3-dioxo-2,3-dihydro-1H-isoindol-5-yl]amino]butyl)azetidine-3-sulfonamide formic acid (compound D38 formic acid)

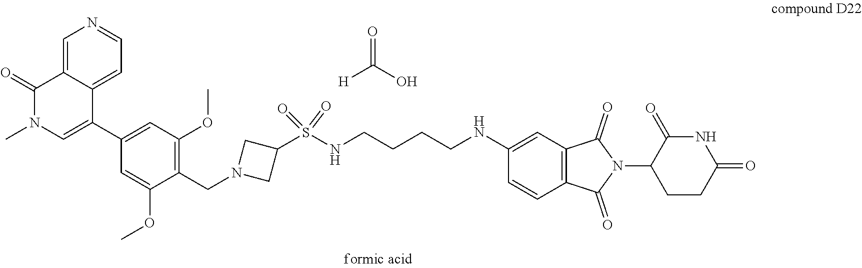

A mixture of N-(4-[[2-(2,6-dioxopiperidin-3-yl)-1,3-dioxo-2,3-dihydro-1H-isoindol-5-yl]amino]butyl) azetidine-3-sulfonamide (64.17 mg, 0.138 mmol, 1.00 equiv) and 2,6-dimethoxy-4-(2-methyl-1-oxo-1,2-dihydro-2,7-naphthyridin-4-yl)benzaldehyde (44.90 mg, 0.138 mmol, 1.00 equiv) in DMF (2 mL) was stirred at room temperature, then adjusted to pH 8-9 by addition of TEA. The above mixture was added NaBH₃CN (26.10 mg, 0.415 mmol, 3.00 equiv) in portions, the resulting mixture was stirred for 2 hours at room temperature. The resulting mixture was concentrated under reduced pressure, the residue was purified by Prep-HPLC (condition: X Select CSH Prep C18 OBD Column, 5 μm, 19*150 mm; mobile phase, Water (0.1% FA) and ACN (15% Phase B up to 30% in 14 minutes); Detector, UV). This resulted in 15 mg (12.59%) of 1-[[2,6-dimethoxy-4-(2-methyl-1-oxo-1,2-dihydro-2,7-naphthyridin-4-yl)phenyl]methyl]-N-(4-[[2-(2,6-dioxopiperidin-3-yl)-1,3-dioxo-2,3-dihydro-1H-isoindol-5-yl]amino]butyl)azetidine-3-sulfonamide formic acid as a yellow solid. ¹H NMR (400 MHz, DMSO-d6) δ 11.07 (s, 1H), 9.45 (s, 1H), 8.73 (d, J=5.7 Hz, 1H), 8.14 (s, 0.5H, FA), 7.87 (s, 1H), 7.59-7.52 (m, 2H), 7.13 (s, 1H), 6.94 (s, 1H), 6.84 (d, J=8.6 Hz, 1H), 6.78 (s, 2H), 6.55 (s, 1H), 5.03 (dd, J=12.9, 5.4 Hz, 1H), 3.84 (s, 7H), 3.60 (s, 4H), 3.28-3.20 (m, 3H), 3.16 (d, J=6.3 Hz, 3H), 2.97 (d, J=6.5 Hz, 2H), 2.92-2.81 (m, 1H), 2.61-2.53 (m, 3H), 2.03-1.95 (m, 1H), 1.55 (s, 4H). LCMS (ESI) m/z: [M+H]⁺=772.30.

### Example 29—Preparation of 1-[[2,6-dimethoxy-4-(2-methyl-1-oxo-1,2-dihydro-2,7-naphthyridin-4-yl)phenyl]methyl]-N-(5-[[2-(2,6-dioxopiperidin-3-yl)-1,3-dioxo-2,3-dihydro-1H-isoindol-5-yl]amino]pentyl) azetidine-3-sulfonamide formic acid (compound D23 formic acid)

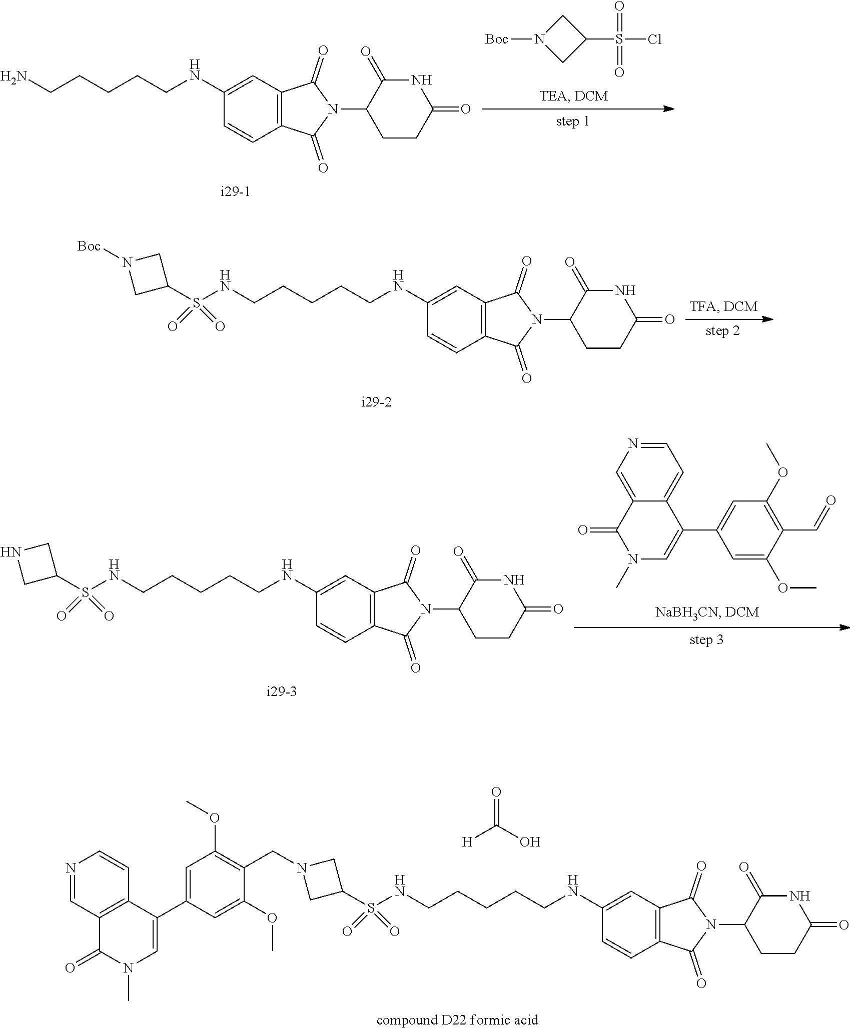

### Step 1: Preparation of tert-butyl-3-[(5-[[2-(2,6-dioxopiperidin-3-yl)-1,3-dioxoisoindol-5-yl]amino]pentyl) sulfamoyl]azetidine-1-carboxylate (i28-2)

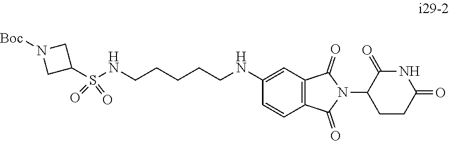 [SMILES: CC(C)(C)OC(=O)N1CC(S(=O)(=O)NCCCCCNc2ccc3c(c2)C(=O)N(C2CCC(=O)NC2=O)C3=O)C1]

To a stirred mixture of 5-[(5-aminopentyl)amino]-2-(2,6-dioxopiperidin-3-yl)isoindole-1,3-dione (100.00 mg, 0.279 mmol, 1.00 equiv) and tert-butyl 3-(chlorosulfonyl)azetidine-1-carboxylate (178.37 mg, 0.698 mmol, 2.50 equiv) in DCM (2.00 mL) was added TEA (84.70 mg, 0.837 mmol, 3.00 equiv). After stirring for 1.5 hours at room temperature, the resulting mixture was concentrated under reduced pressure. The residue was purified by Prep-TLC (CH₂Cl₂/EA (1:2)) to afford tert-butyl-3-[(5-[[2-(2,6-dioxopiperidin-3-yl)-1,3-dioxoisoindol-5-yl]amino]pentyl)sulfamoyl]azetidine-1-carboxylate (58.7 mg, 33.87%) as a yellow solid. LCMS (ESI) m/z: [M+H]+=578.

### Step 2: Preparation of N-(5-((2-(2,6-dioxopiperidin-3-yl)-1,3-dioxoisoindolin-5-yl)amino)pentyl)azetidine-3-sulfonamide (i28-3)

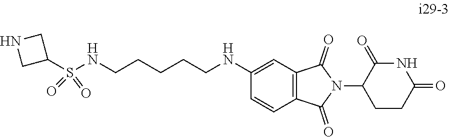 [SMILES: O=C1CCC(N2C(=O)c3ccc(NCCCCCNS(=O)(=O)C4CNC4)cc3C2=O)C(=O)N1]

To a stirred mixture of tert-butyl 3-[(5-[[2-(2,6-dioxopiperidin-3-yl)-1,3-dioxoisoindol-5-yl]amino]pentyl)sulfamoyl]azetidine-1-carboxylate (58.70 mg, 0.102 mmol, 1.00 equiv) in DCM (2.00 mL) was added TFA (0.40 mL, 5.385 mmol, 52.99 equiv). After stirring for 1 hour at room temperature, the resulting mixture was concentrated under reduced pressure. The residue was used in the next step directly without further purification. LCMS (ESI) m/z: [M+H]⁺=478.17.

### Step 3: Preparation of 1-[[2,6-dimethoxy-4-(2-methyl-1-oxo-1,2-dihydro-2,7-naphthyridin-4-yl)phenyl]methyl]-N-(5-[[2-(2,6-dioxopiperidin-3-yl)-1,3-dioxo-2,3-dihydro-1H-isoindol-5-yl]amino]pentyl)azetidine-3-sulfonamide formic acid (compound D22 formic acid)

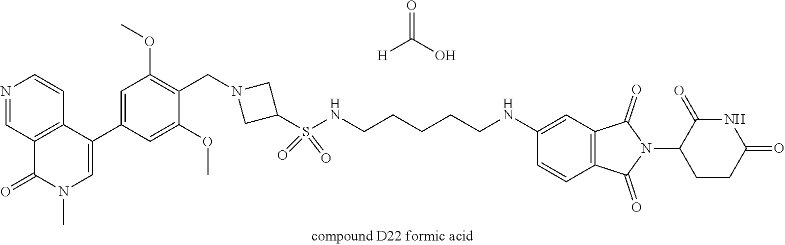 [SMILES: COc1cc(-c2cn(C)c(=O)c3cnccc23)cc(OC)c1CN1CC(S(=O)(=O)NCCCCCNc2ccc3c(c2)C(=O)N(C2CCC(=O)NC2=O)C3=O)C1 ; O=CO]

A mixture of N-(5-[[2-(2,6-dioxopiperidin-3-yl)-1,3-dioxo-2,3-dihydro-1H-isoindol-5-yl]amino]pentyl) azetidine-3-sulfonamide (48.54 mg, 0.102 mmol, 1.00 equiv) and 2,6-dimethoxy-4-(2-methyl-1-oxo-1,2-dihydro-2,7-naphthyridin-4-yl)benzaldehyde (39.56 mg, 0.122 mmol, 1.20 equiv) in THE (2 mL) was stirred at room temperature, then adjusted to pH 8-˜9 with TEA. To the above mixture was added NaBH₃CN (12.78 mg, 0.203 mmol, 2.00 equiv) in portions, and the resulting mixture was stirred for 2 hours at room temperature. The resulting mixture was concentrated under reduced pressure, and the residue was purified by Prep-HPLC (conditions: Sun Fire C18 OBD Prep Column, 19 mm×250 mm; mobile phase, Water (0.1% FA) and ACN (hold 3% Phase B in 2 minutes, up to 15% in 8 minutes); Detector, UV). This resulted in 7.4 mg (8.31%) of 1-[[2,6-dimethoxy-4-(2-methyl-1-oxo-1,2-dihydro-2,7-naphthyridin-4-yl)phenyl]methyl]-N-(5-[[2-(2,6-dioxopiperidin-3-yl)-1,3-dioxo-2,3-dihydro-1H-isoindol-5-yl]amino]pentyl)azetidine-3-sulfonamide formic acid as a yellow solid. ¹H NMR (400 MHz, DMSO-d6) δ 11.07 (s, 1H), 9.44 (s, 1H), 8.72 (d, J=5.7 Hz, 1H), 7.86 (s, 1H), 7.59-7.52 (m, 2H), 7.21 (s, 1H), 7.11 (s, 1H), 6.93 (s, 1H), 6.83 (dd, J=8.3, 1.7 Hz, 1H), 6.76 (s, 2H), 6.55 (s, 1H), 5.03 (dd, J=13.0, 5.4 Hz, 1H), 4.02 (s, 1H), 3.83 (s, 6H), 3.60 (s, 4H), 3.29-3.20 (m, 2H), 3.19-3.08 (m, 3H), 3.01-2.78 (m, 4H), 2.61-2.51 (m, 3H), 2.06-1.93 (t, J=12.7 Hz, 1H), 1.60-1.51 (m, 2H), 1.50-1.42 (m, 2H), 1.42-1.32 (m, 2H). LCMS (ESI) m/z: [M+H]⁺=786.28.

### Example 30—Preparation of 1-[[2,6-dimethoxy-4-(2-methyl-1-oxo-2,7-naphthyridin-4-yl)phenyl]methyl]-N-(2-[4-[2-(2,6-dioxopiperidin-3-yl)-1,3-dioxoisoindol-5-yl]piperazin-1-yl]ethyl)azetidine-3-sulfonamide formic acid (compound D24 formic acid)

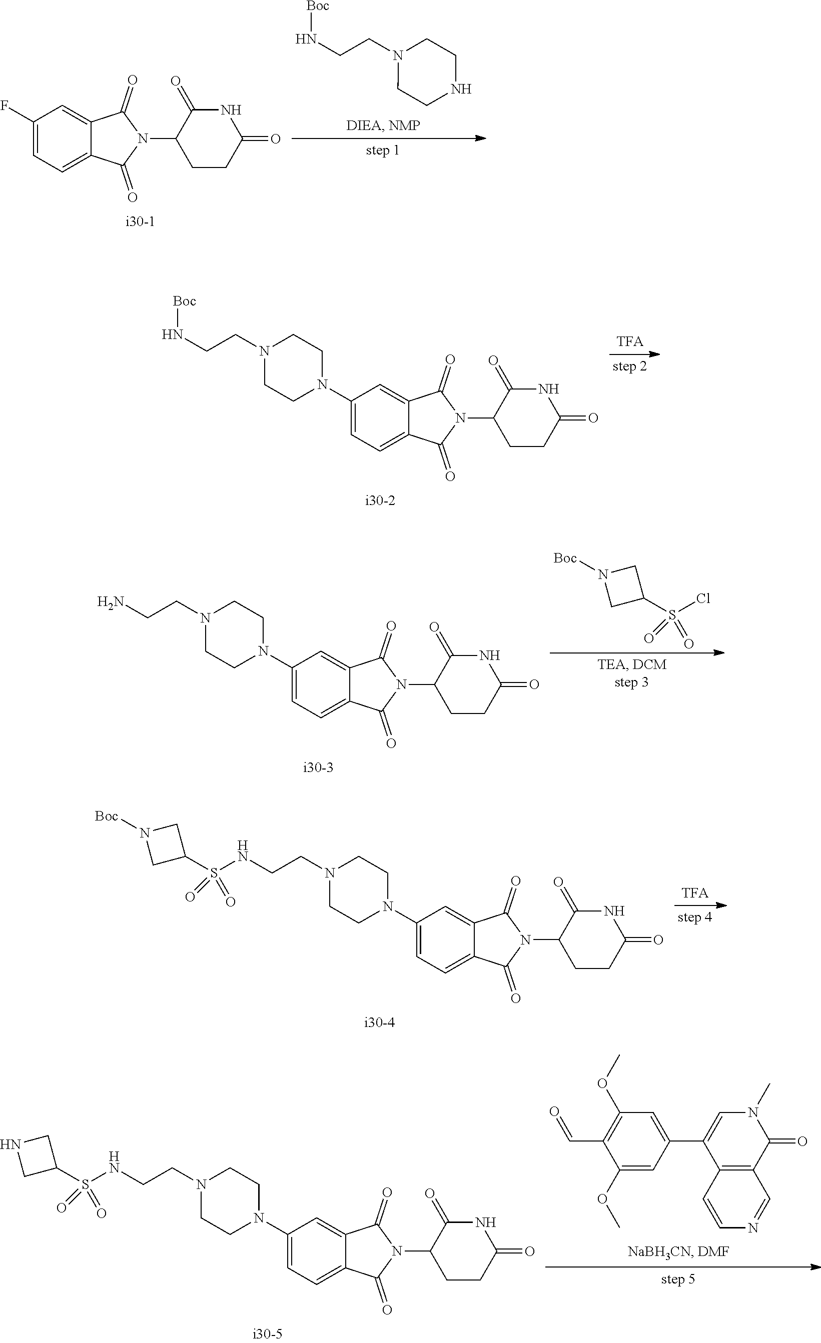

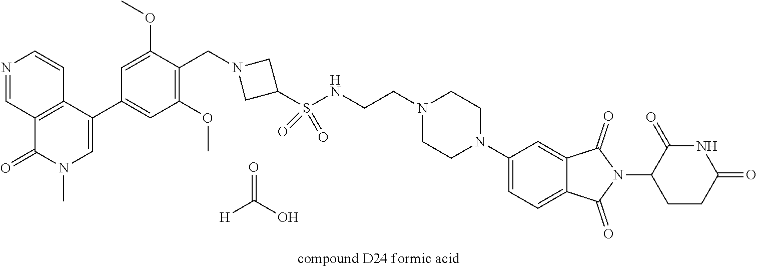

### Step 1: Preparation of tert-butyl N-(2-[4-[2-(2,6-dioxopiperidin-3-yl)-1,3-dioxoisoindol-5-yl]piperazin-1-yl]ethyl)carbamate (i30-2)

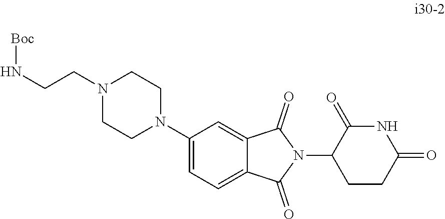 [SMILES: CC(C)(C)OC(=O)NCCN1CCN(c2ccc3c(c2)C(=O)N(C2CCC(=O)NC2=O)C3=O)CC1]

To a stirred solution of 2-(2,6-dioxopiperidin-3-yl)-5-fluoroisoindole-1,3-dione (1.50 g, 5.430 mmol, 1.00 equiv) and tert-butyl N-[2-(piperazin-1-yl)ethyl]carbamate (1.49 g, 6.516 mmol, 1.20 equiv) in NMP (10.00 mL) was added DIEA (1.40 g, 10.861 mmol, 2.00 equiv) dropwise at room temperature. The resulting mixture was stirred for 6 hours at 90° C. under nitrogen atmosphere. The residue was purified by reverse flash chromatography (conditions: column, C18 silica gel; mobile phase, ACN in water, 10% to 50% gradient in 20 minutes; detector, UV 254 nm). This resulted in tert-butyl N-(2-[4-[2-(2,6-dioxopiperidin-3-yl)-1,3-dioxoisoindol-5-yl]piperazin-1-yl]ethyl)carbamate (2 g, 75.85%) as a green oil. LCMS (ESI) m/z: [M+H]+=486.

### Step 2: Preparation of 5-[4-(2-aminoethyl)piperazin-1-yl]-2-(2,6-dioxopiperidin-3-yl)isoindole-1,3-dione (i30-3)

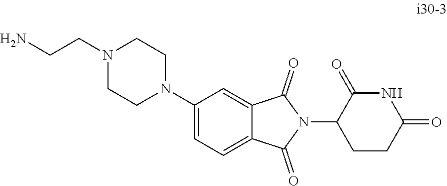 [SMILES: NCCN1CCN(c2ccc3c(c2)C(=O)N(C2CCC(=O)NC2=O)C3=O)CC1]

A solution of tert-butyl N-(2-[4-[2-(2,6-dioxopiperidin-3-yl)-1,3-dioxoisoindol-5-yl]piperazin-1-yl]ethyl) carbamate (2.00 g, 4.119 mmol, 1.00 equiv) and TFA (2.00 mL, 26.926 mmol, 6.54 equiv) in DCM (5.00 mL, 78.650 mmol, 19.09 equiv) was stirred for 1 hours at room temperature. The resulting mixture was concentrated under vacuum. This resulted in 5-[4-(2-aminoethyl)piperazin-1-yl]-2-(2,6-dioxopiperidin-3-yl)isoindole-1,3-dione (1.5 g, 94.48%) as a green solid. LCMS (ESI) m/z: [M+H]⁺=386.

### Step 3: Preparation of tert-butyl 3-[(2-[4-[2-(2,6-dioxopiperidin-3-yl)-1,3-dioxoisoindol-5-yl]piperazin-1-yl]ethyl)sulfamoyl]azetidine-1-carboxylate (i30-4)

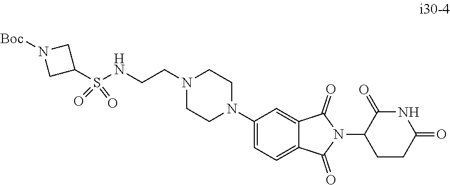 [SMILES: CC(C)(C)OC(=O)N1CC(S(=O)(=O)NCCN2CCN(c3ccc4c(c3)C(=O)N(C3CCC(=O)NC3=O)C4=O)CC2)C1]

To a stirred solution of 5-[4-(2-aminoethyl)piperazin-1-yl]-2-(2,6-dioxopiperidin-3-yl)isoindole-1,3-dione (400.00 mg, 1.038 mmol, 1.00 equiv) and tert-butyl 3-(chlorosulfonyl)azetidine-1-carboxylate (318.46 mg, 1.245 mmol, 1.20 equiv) in DCM (10.00 mL) was added TEA (210.03 mg, 2.076 mmol, 2.00 equiv) at room temperature. The resulting mixture was stirred for 2 hours at room temperature. The resulting mixture was concentrated under vacuum. The residue was purified by silica gel column chromatography, eluted with DCM/EtOAc (1:1) to afford tert-butyl 3-[(2-[4-[2-(2,6-dioxopiperidin-3-yl)-1,3-dioxoisoindol-5-yl]piperazin-1-yl]ethyl)sulfamoyl]azetidine-1-carboxylate (500 mg, 79.68%) as a green solid. LCMS (ESI) m/z: [M+H]⁺=605.

### Step 4: Preparation of N-(2-[4-[2-(2,6-dioxopiperidin-3-yl)-1,3-dioxoisoindol-5-yl]piperazin-1-yl]ethyl)azetidine-3-sulfonamide (i30-5)

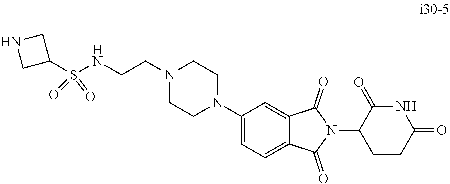 [SMILES: O=C1CCC(N2C(=O)c3ccc(N4CCN(CCNS(=O)(=O)C5CNC5)CC4)cc3C2=O)C(=O)N1]

A solution of tert-butyl 3-[(2-[4-[2-(2,6-dioxopiperidin-3-yl)-1,3-dioxoisoindol-5-yl]piperazin-1-yl]ethyl) sulfamoyl]azetidine-1-carboxylate (500.00 mg, 0.827 mmol, 1.00 equiv) and TFA (3.00 mL) in DCM (5.00 mL) was stirred for 1 hour at room temperature. The resulting mixture was concentrated under vacuum. This resulted in N-(2-[4-[2-(2,6-dioxopiperidin-3-yl)-1,3-dioxoisoindol-5-yl]piperazin-1-yl]ethyl)azetidine-3-sulfonamide (400 mg, 95.87%) as a green solid. LCMS (ESI) m/z: [M+H]⁺=505.

### Step 5: Preparation of 1-[[2,6-dimethoxy-4-(2-methyl-1-oxo-2,7-naphthyridin-4-yl)phenyl]methyl]-N-(2-[4-[2-(2,6-dioxopiperidin-3-yl)-1,3-dioxoisoindol-5-yl]piperazin-1-yl]ethyl)azetidine-3-sulfonamide formic acid (compound D24 formic acid)

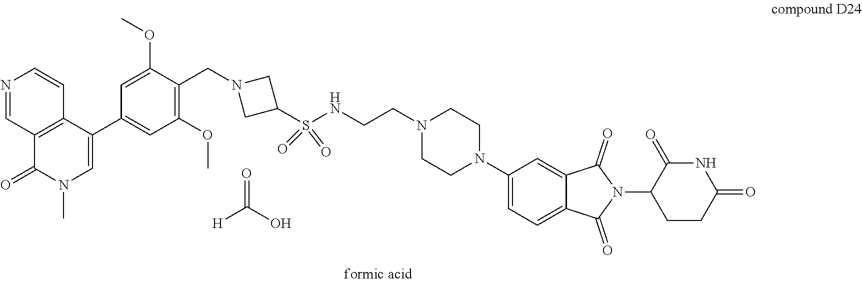

A solution of N-(2-[4-[2-(2,6-dioxopiperidin-3-yl)-1,3-dioxoisoindol-5-yl]piperazin-1-yl]ethyl)azetidine-3-sulfonamide (60.00 mg, 0.119 mmol, 1.00 equiv) and 2,6-dimethoxy-4-(2-methyl-1-oxo-2,7-naphthyridin-4-yl)benzaldehyde (46.28 mg, 0.143 mmol, 1.20 equiv) in DMF (1.50 mL) was stirred for 20 minutes at room temperature. Then NaBH₃CN (14.95 mg, 0.238 mmol, 2.00 equiv) was added to the reaction mixture. The resulting mixture was stirred for 1 hour at room temperature. The residue was purified by reverse flash chromatography (conditions: column, C18 silica gel; mobile phase, ACN in water, 10% to 50% gradient in 20 minutes; detector, UV 254 nm). This resulted in 1-[[2,6-dimethoxy-4-(2-methyl-1-oxo-2,7-naphthyridin-4-yl)phenyl]methyl]-N-(2-[4-[2-(2,6-dioxopiperidin-3-yl)-1,3-dioxoisoindol-5-yl]piperazin-1-yl]ethyl)azetidine-3-sulfonamide (9.4 mg, 9.72%) as a green solid. ¹H NMR (400 MHz, DMSO-d6) δ 12.79 (brs, 0.8H, FA(COOH)), 11.08 (s, 1H), 9.44 (s, 1H), 8.71 (d, J=5.7 Hz, 1H), 8.14 (s, 0.8H, FA), 7.86 (s, 1H), 7.66 (d, J=8.5 Hz, 1H), 7.56 (d, J=5.8 Hz, 1H), 7.33 (d, J=2.3 Hz, 1H), 7.24 (dd, J=8.8, 2.3 Hz, 1H), 7.11 (s, 1H), 6.73 (s, 2H), 5.07 (dd, J=13.0, 5.4 Hz, 1H), 4.08-4.02 (m, 1H), 3.82 (s, 7H), 3.69-3.62 (m, 2H), 3.60 (s, 3H), 3.50-3.39 (m, 8H), 3.12-3.05 (m, 2H), 2.95-2.83 (m, 1H), 2.63-2.55 (m, 3H), 2.55 (s, 2H), 2.47-2.39 (m, 3H), 2.07-1.98 (m, 1H). LCMS (ESI) m/z: [M+H]⁺=813.30.

### Example 31—Preparation of (2S)-1-[[2,6-dimethoxy-4-(2-methyl-1-oxo-1,2-dihydro-2,7-naphthyridin-4-yl)phenyl]methyl]-N-[2-[(2-[[2-(2,6-dioxopiperidin-3-yl)-1,3-dioxo-2,3-dihydro-1H-isoindol-4-yl]amino]ethyl)(methyl)amino]ethyl]azetidine-2-carboxamide (compound D25)

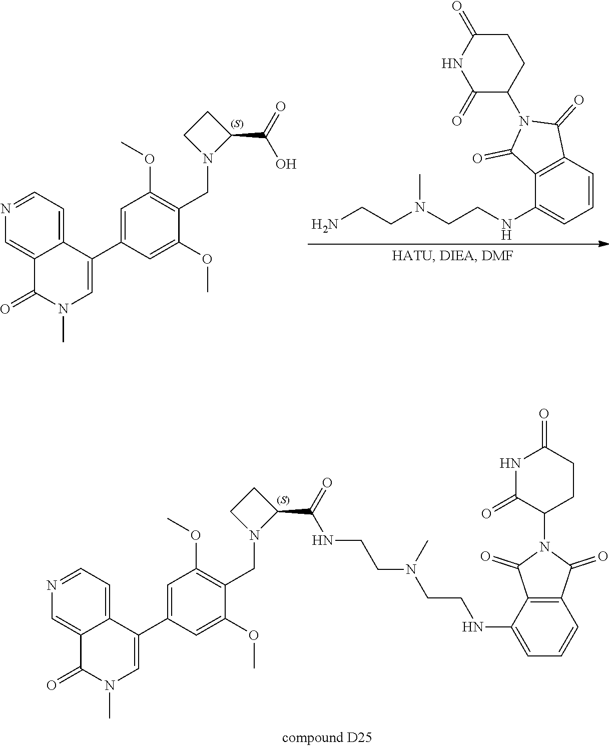

To a solution of (2S)-1-[[2,6-dimethoxy-4-(2-methyl-1-oxo-1,2-dihydro-2,7-naphthyridin-4-yl)phenyl]methyl]azetidine-2-carboxylic acid (80 mg, 0.195 mmol, 1.00 equiv) and DIEA (75.8 mg, 0.586 mmol, 3.00 equiv) in DMF (1.50 mL) was added HATU (111.4 mg, 0.293 mmol, 1.50 equiv), and the resulting solution was stirred at room temperature for 1 hour. The crude mixture was directly purified by Prep-HPLC (conditions: SunFire C18 OBD Prep Column, 100 A, 5 μm, 19 mm×250 mm; Mobile Phase A: Water (0.1% FA), Mobile Phase B: ACN; Flow rate: 25 mL/minuteute; Gradient: 7% B to 22% B in 8 minutes; 254 nm; Rₜ: 7.75 minutes) to afford (2S)-1-[[2,6-dimethoxy-4-(2-methyl-1-oxo-1,2-dihydro-2,7-naphthyridin-4-yl)phenyl]methyl]-N-[2-[(2-[[2-(2,6-dioxopiperidin-3-yl)-1,3-dioxo-2,3-dihydro-1H-isoindol-4-yl]amino]ethyl)(methyl)amino]ethyl]azetidine-2-carboxamide (5.5 mg, 3.5%) as a yellow solid. ¹H NMR (300 MHz, Methanol-d4) δ 9.45 (d, J=1.1 Hz, 1H), 8.67 (d, J=5.8 Hz, 1H), 7.72 (s, 1H), 7.63 (d, J=5.9 Hz, 1H), 7.54-7.42 (m, 1H), 6.99 (d, J=7.1 Hz, 1H), 6.91 (dd, J=8.5, 3.1 Hz, 1H), 6.71 (d, J=0.9 Hz, 2H), 5.13-5.02 (m, 1H), 3.86 (s, 8H), 3.66 (d, J=1.0 Hz, 5H), 3.28 (s, 5H), 2.76-2.66 (m, 6H), 2.53-2.42 (m, 2H), 2.34 (s, 3H), 2.30-2.19 (m, 1H), 2.15-1.94 (m, 2H). LCMS (ESI) m/z: [M+H]⁺=765.30.

### Example 32—Preparation of N-[2-[(2-[[(2S)-1-[[2,6-dimethoxy-4-(2-methyl-1-oxo-1,2-dihydro-2,7-naphthyridin-4-yl)phenyl]methyl]azetidin-2-yl]formamido]ethyl)(methyl)amino]ethyl]-2-[[2-(2,6-dioxopiperidin-3-yl)-1,3-dioxo-2,3-dihydro-1H-isoindol-5-yl]oxy]acetamide (compound D26)

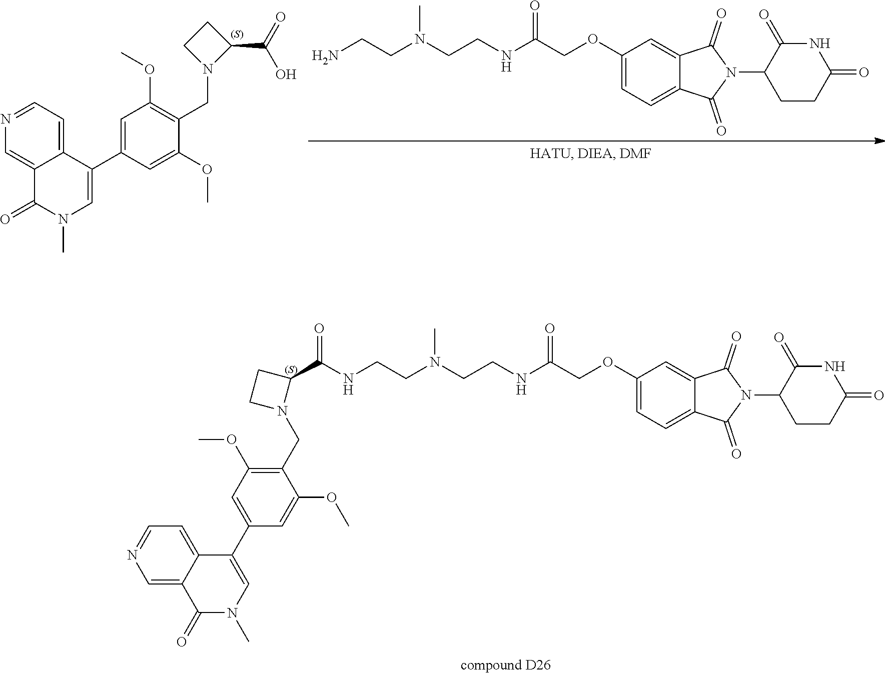

To a solution of (2S)-1-[[2,6-dimethoxy-4-(2-methyl-1-oxo-1,2-dihydro-2,7-naphthyridin-4-yl)phenyl]methyl]azetidine-2-carboxylic acid (30 mg, 0.073 mmol, 1.00 equiv) and DIEA (28.4 mg, 0.220 mmol, 3.00 equiv) in DMF (1.00 mL) was added HATU (41.8 mg, 0.110 mmol, 1.50 equiv) and N-[2-[(2-aminoethyl)(methyl)amino]ethyl]-2-[[2-(2,6-dioxopiperidin-3-yl)-1,3-dioxo-2,3-dihydro-1H-isoindol-5-yl]oxy]acetamide (31.61 mg, 0.073 mmol, 1.00 equiv). The resulting solution was stirred at room temperature for 1 hour. The crude mixture was directly purified by Prep-HPLC (condition: SunFire C18 OBD Prep Column, 100 A, 5 μm, 19 mm×250 mm; Mobile Phase A: Water (0.1% FA), Mobile Phase B: ACN; Flow rate: 25 mL/minuteute; Gradient: 5% B to 5% B in 2 minutes; 254 nm; Rₜ: 9.88 minutes) to afford N-[2-[(2-[[(2S)-1-[[2,6-dimethoxy-4-(2-methyl-1-oxo-1,2-dihydro-2,7-naphthyridin-4-yl)phenyl]methyl]azetidin-2-yl]formamido]ethyl)(methyl)amino]ethyl]-2-[[2-(2,6-dioxopiperidin-3-yl)-1,3-dioxo-2,3-dihydro-1H-isoindol-5-yl]oxy]acetamide (4.8 mg, 7.5%) as a yellow solid. ¹H NMR (300 MHz, Acetonitrile-d3) 6 9.52 (s, 1H), 9.11 (s, 1H), 8.70 (d, J=5.7 Hz, 1H), 8.20-8.02 (m, 1H), 7.79 (t, J=6.5 Hz, 2H), 7.57 (d, J=5.0 Hz, 2H), 7.45-7.23 (m, 2H), 6.73 (s, 2H), 4.99 (dd, J=12.1, 5.3 Hz, 1H), 4.63 (s, 2H), 4.38 (s, 1H), 4.11 (s, 2H), 3.87 (s, 6H), 3.72-3.60 (m, 5H), 3.59-3.49 (m, 2H), 3.45 (d, J=5.6 Hz, 2H), 3.01 (dt, J=11.1, 5.7 Hz, 4H), 2.83-2.72 (m, 2H), 2.72-2.60 (m, 5H), 2.13 (ddd, J=10.6, 5.5, 3.1 Hz, 2H). LCMS (ESI) m/z: [M+H]+=823.45.

### Example 33—Preparation of 4-(((((S)-1-(2,6-dimethoxy-4-(2-methyl-1-oxo-1,2-dihydro-2,7-naphthyridin-4-yl)benzyl)azetidin-2-yl)methyl)(methyl)amino)methyl)-2-(2,6-dioxopiperidin-3-yl)isoindoline-1,3-dione (compound D27)

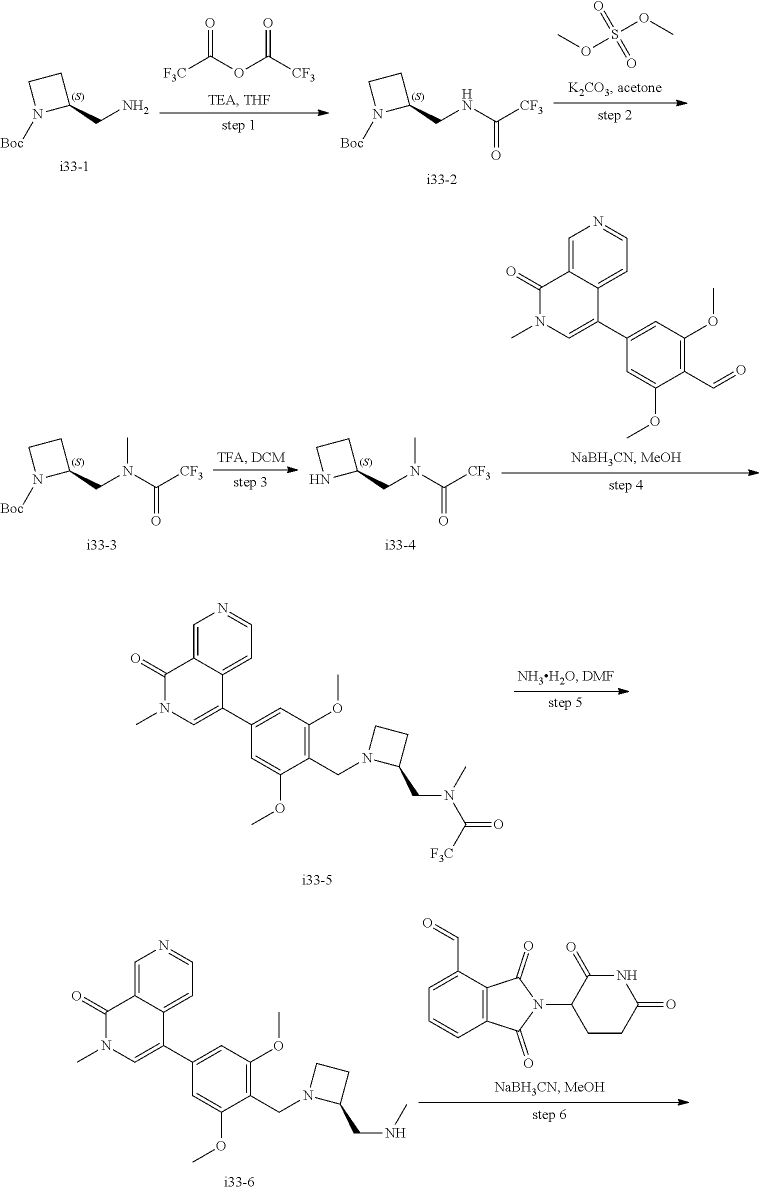

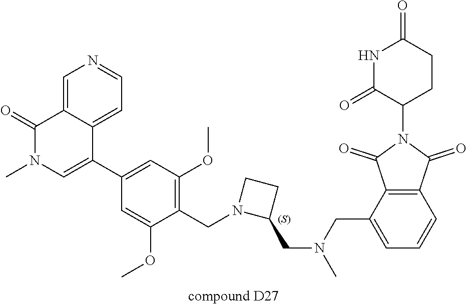

### Step 1: Preparation of tert-butyl (2S)-2-((2,2,2-trifluoroacetamido)methyl)azetidine-1-carboxylate (i33-2)

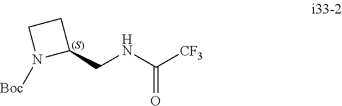

To a solution of tert-butyl (2S)-2-(aminomethyl)azetidine-1-carboxylate (900.00 mg, 4.832 mmol, 1.00 equiv) and trifluoroacetic anhydride (1522.33 mg, 7.248 mmol, 1.5 equiv) in THE (9.00 mL) was added TEA (977.92 mg, 9.664 mmol, 2 equiv). The mixture was stirred at 25° C. for 12 hours. The resulting solution was diluted with EA. Then washed with water (3×50 mL). The residue was applied onto a silica gel column with ethyl EA/PE (15/85). The resulting mixture were evaporated to dryness to afford tert-butyl (2S)-2-[(2,2,2-trifluoroacetamido) methyl]azetidine-1-carboxylate (1270 mg, 93.11%) as a yellow oil. LCMS (ESI) m/z: [M+H]⁺=283.

### Step 2: Preparation of tert-butyl (2S)-2-[(2,2,2-trifluoro-N-methylacetamido)methyl]azetidine-1-carboxylate (i33-3)

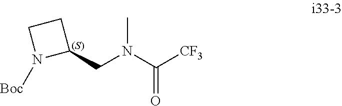

To a solution of tert-butyl (2S)-2-[(2,2,2-trifluoroacetamido)methyl]azetidine-1-carboxylate (1270.00 mg, 4.499 mmol, 1.00 equiv) and dimethyl sulfate (681.00 mg, 5.399 mmol, 1.2 equiv) in acetone (15.00 mL) was added K₂CO₃ (621.83 mg, 4.499 mmol, 1 equiv). The mixture was stirred at 25° C. for 12 hours. The resulting mixture were evaporated to dryness to afford tert-butyl (2S)-2-[(2,2,2-trifluoro-N-methylacetamido)methyl]azetidine-1-carboxylate (1640 mg, 123.02%) as a yellow oil that was used directly without further purification. LCMS (ESI) m/z: [M+H]⁺=297.

### Step 3: Preparation of N-[(2S)-azetidin-2-ylmethyl]-2,2,2-trifluoro-N-methylacetamide (i33-4)

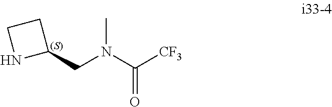

A solution of tert-butyl (2S)-2-[(2,2,2-trifluoro-N-methylacetamido)methyl]azetidine-1-carboxylate (1.64 g, 5.535 mmol, 1.00 equiv) and TFA (3.50 mL, 47.121 mmol, 8.51 equiv) in DCM (16.00 mL) was stirred for 1 hour at 25° C. The mixture was concentrated to give N-[(2S)-azetidin-2-ylmethyl]-2,2,2-trifluoro-N-methylacetamide (2.08 g) as a brown oil that was used directly without further purification. LCMS (ESI) m/z: [M+H]+=197.

### Step 4: Preparation of N-[[(2S)-1-[[2,6-dimethoxy-4-(2-methyl-1-oxo-2,7-naphthyridin-4-yl)phenyl]methyl]azetidin-2-yl]methyl]-2,2,2-trifluoro-N-methylacetamide (i33-5)

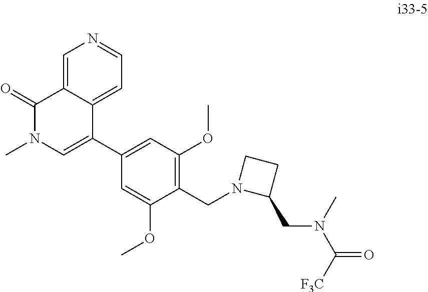 [SMILES: COc1cc(-c2cn(C)c(=O)c3cnccc23)cc(OC)c1CN1CC[C@H]1CN(C)C(=O)C(F)(F)F]

To a solution of 2,6-dimethoxy-4-(2-methyl-1-oxo-2,7-naphthyridin-4-yl)benzaldehyde (552.00 mg, 1.702 mmol, 1.00 equiv) and N-[(2S)-azetidin-2-ylmethyl]-2,2,2-trifluoro-N-methylacetamide(500.81 mg, 2.553 mmol, 1.50 equiv) in DMF (6.00 mL) was added NaBH(OAc)₃ (721.42 mg, 3.404 mmol, 2.00 equiv). The resulting solution was stirred at 25° C. for 1 hour. The mixture was concentrated to give crude product that was purified by chromatography on silica gel eluted with MeOH/DCM (5:95) to give N-[[(2S)-1-[[2,6-dimethoxy-4-(2-methyl-1-oxo-2,7-naphthyridin-4-yl)phenyl]methyl]azetidin-2-yl]methyl]-2,2,2-trifluoro-N-methylacetamide (275 mg, 32.03%) as an off-white solid. LCMS (ESI) m/z: [M+H]⁺=505.

### Step 5: Preparation of (S)-4-(3,5-dimethoxy-4-((2-((methylamino)methyl)azetidin-1-yl)methyl)phenyl)-2-methyl-2,7-naphthyridin-1(2H)-one (i33-6)

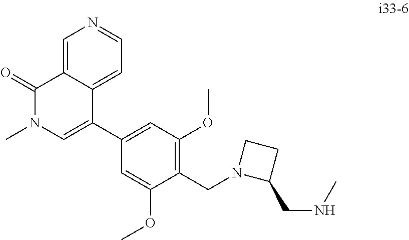 [SMILES: CNC[C@@H]1CCN1Cc1c(OC)cc(-c2cn(C)c(=O)c3cnccc23)cc1OC]

A solution of N-[[(2R)-1-[[2,6-dimethoxy-4-(2-methyl-1-oxo-2,7-naphthyridin-4-yl)phenyl]methyl]azetidin-2-yl]methyl]-2,2,2-trifluoro-N-methylacetamide (230 mg, 0.456 mmol, 1.00 equiv) and NH₃·H₂O (1 mL, 0.008 mmol, 0.05 equiv) in DMF (2.50 mL) was stirred at 25° C. for 1 hour. The resulting mixture were evaporated to dryness to afford 4-(3,5-dimethoxy-4-[[(2R)-2-[(methylamino) methyl]azetidin-1-yl]methyl]phenyl)-2-methyl-2,7-naphthyridin-1-one(219 mg) as a brown oil that was used directly without further purification. LCMS (ESI) m/z: [M+H]+=409.

### Step 6: Preparation of 4-(((((S)-1-(2,6-dimethoxy-4-(2-methyl-1-oxo-1,2-dihydro-2,7-naphthyridin-4-yl)benzyl)azetidin-2-yl)methyl)(methyl)amino)methyl)-2-(2,6-dioxopiperidin-3-yl)isoindoline-1,3-dione (compound D27)

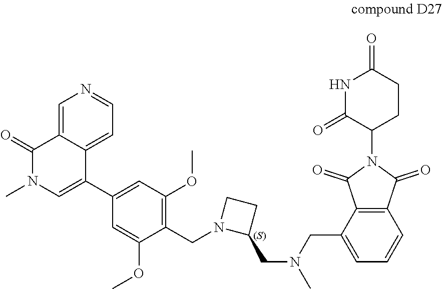

To a stirred solution of 4-(3,5-dimethoxy-4-[[(2R)-2-[(methylamino)methyl]azetidin-1-yl]methyl]phenyl)-2-methyl-1,2-dihydro-2,7-naphthyridin-1-one (150.00 mg, 0.367 mmol, 1.00 equiv) and 2-(2,6-dioxopiperidin-3-yl)-1,3-dioxo-2,3-dihydro-1H-isoindole-4-carbaldehyde (105.11 mg, 0.367 mmol, 1.00 equiv) in MeOH (2.00 mL) was added NaBH₃CN (115.38 mg, 1.836 mmol, 5 equiv). The mixture was stirred at 25° C. for 1 hour. Without any additional work-up, the mixture was purified by prep-HPLC (conditions: SunFire C18 OBD Prep Column, 100A, 5 μm, 19 mm×250 mm; Mobile Phase A: Water (0.05% TFA), Mobile Phase B: ACN; Flow rate: 25 mL/minute; Gradient: 3% B to 3% B in 2 minutes; 254 nm; Rₜ: 14.55 minutes) to give 4-(((((S)-1-(2,6-dimethoxy-4-(2-methyl-1-oxo-1,2-dihydro-2,7-naphthyridin-4-yl)benzyl)azetidin-2-yl)methyl)(methyl)amino)methyl)-2-(2,6-dioxopiperidin-3-yl)isoindoline-1,3-dione (8.0 mg, 3.01%) as a yellow solid. ¹H NMR (400 MHz, Methanol-d4) δ 9.54 (s, 1H), 8.67 (d, J=5.7 Hz, 1H), 8.57 (s, 0.4H, FA), 7.91-7.86 (m, 1H), 7.84 (d, J=6.0 Hz, 2H), 7.74 (d, J=6.5 Hz, 1H), 7.57 (t, J=6.3 Hz, 1H), 6.84 (d, J=5.4 Hz, 2H), 5.20-5.08 (m, 1H), 4.72-4.31 (m, 3H), 4.15-3.98 (m, 3H), 3.92 (d, J=11.5 Hz, 6H), 3.71 (d, J=1.8 Hz, 3H), 2.99-2.80 (m, 3H), 2.80-2.49 (m, 4H), 2.38-1.98 (m, 5H). LCMS (ESI) m/z: [M+H]+=679.30.

### Example 34—Preparation of 4-(((1-(2,6-dimethoxy-4-(2-methyl-1-oxo-1,2-dihydro-2,7-naphthyridin-4-yl)benzyl)azetidin-3-yl)(methyl)amino)methyl)-2-(2,6-dioxopiperidin-3-yl)isoindoline-1,3-dione (compound D28)

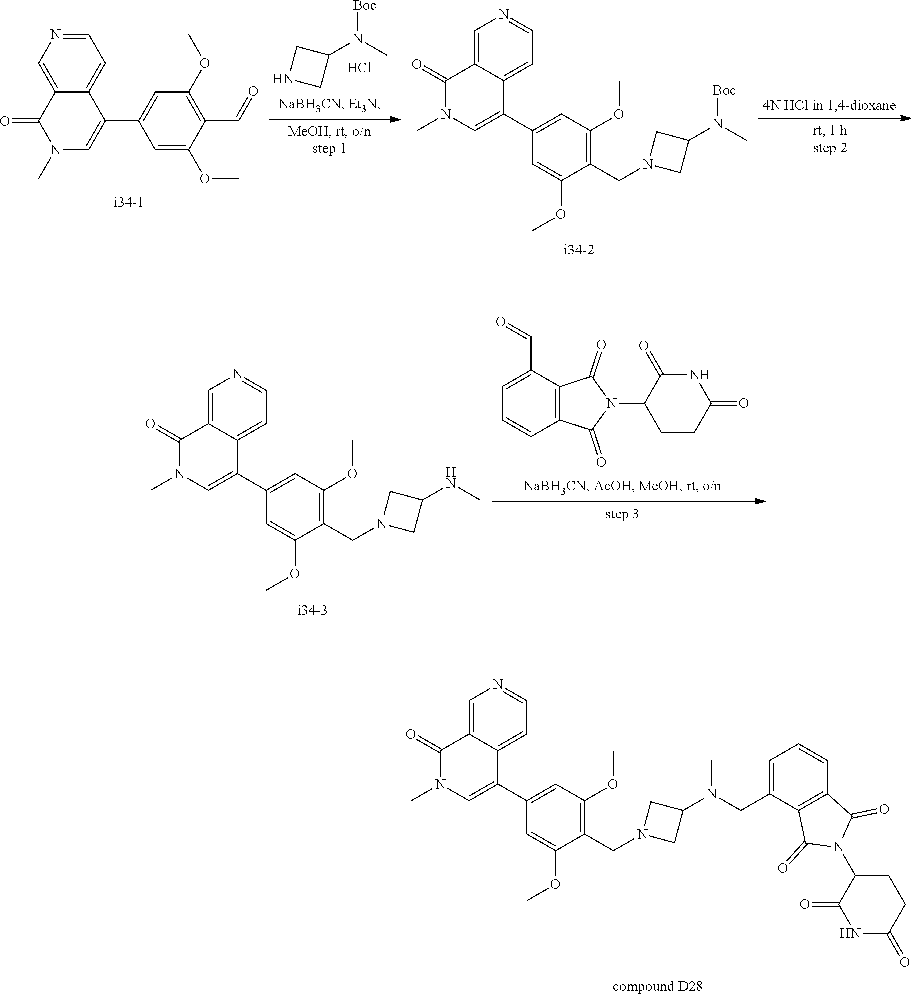

### Step 1: Preparation of tert-butyl (1-(2,6-dimethoxy-4-(2-methyl-1-oxo-1,2-dihydro-2,7-naphthyridin-4-yl)benzyl)azetidin-3-yl)(methyl)carbamate (i34-2)

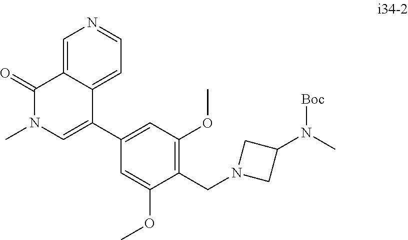 [SMILES: COc1cc(-c2cn(C)c(=O)c3cnccc23)cc(OC)c1CN1CC(N(C)C(=O)OC(C)(C)C)C1]

To a solution of 2,6-dimethoxy-4-(2-methyl-1-oxo-1,2-dihydro-2,7-naphthyridin-4-yl) benzaldehyde (250.00 mg, 0.772 mmol, 1.00 equiv) and tert-butyl azetidin-3-yl(methyl) carbamate hydrochloride (171.38 mg, 0.772 mmol, 1.00 equiv), was added Et₃N (77.97 mg, 0.772 mmol, 1.00 equiv) and NaBH₃CN (97.27 mg, 1.544 mmol, 2.00 equiv). The resulting mixture was stirred overnight. The mixture was concentrated under vacuum. The residue was purified by silica gel column chromatography, eluted with EA in PE from 0% to 40% to afford tert-butyl (1-(2,6-dimethoxy-4-(2-methyl-1-oxo-1,2-dihydro-2,7-naphthyridin-4-yl)benzyl)azetidin-3-yl)(methyl) carbamate (i70 mg, 0.344 mmol, 44.62%) as a white solid. LCMS (ESI) m/z: [M+H]⁺=495.

### Step 2: Preparation of 4-(3,5-dimethoxy-4-((3-(methylamino)azetidin-1-yl)methyl)phenyl)-2-methyl-2,7-naphthyridin-1(2H)-one (i34-3)

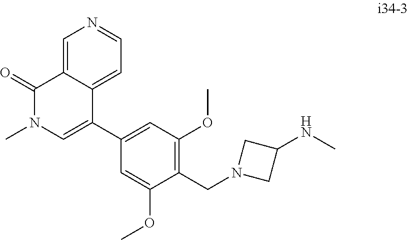 [SMILES: CNC1CN(Cc2c(OC)cc(-c3cn(C)c(=O)c4cnccc34)cc2OC)C1]

Tert-butyl(1-(2,6-dimethoxy-4-(2-methyl-1-oxo-1,2-dihydro-2,7-naphthyridin-4-yl)benzyl) azetidin-3-yl) (methyl) carbamate (i70 mg, 0.344 mmol, 1.00 equiv) was dissolved in 4 N HCl in 1,4-dioxane (5 mL, 20 mmol, 58.13 equiv). The resulting solution was stirred for one hour at room temperature. The resulting mixture was concentrated to afford 4-(3,5-dimethoxy-4-((3-(methylamino)azetidin-1-yl)methyl)phenyl)-2-methyl-2,7-naphthyridin-1(2H)-one (180 mg, crude) as a white solid, that was used directly without further purification. LCMS (ESI) m/z: [M+H]⁺=395.

### Step 3: Preparation of 4-(((1-(2,6-dimethoxy-4-(2-methyl-1-oxo-1,2-dihydro-2,7-naphthyridin-4-yl)benzyl)azetidin-3-yl)(methyl)amino)methyl)-2-(2,6-dioxopiperidin-3-yl)isoindoline-1,3-dione (compound D28)

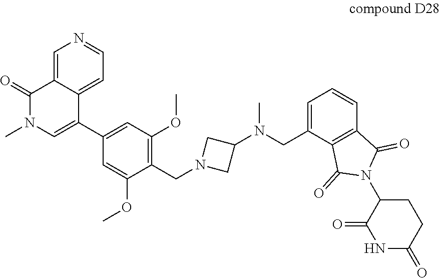 [SMILES: COc1cc(-c2cn(C)c(=O)c3cnccc23)cc(OC)c1CN1CC(N(C)Cc2cccc3c2C(=O)N(C2CCC(=O)NC2=O)C3=O)C1]

To a mixture of 4-(3,5-dimethoxy-4-[[3-(methylamino)azetidin-1-yl]methyl]phenyl)-2-methyl-1,2-dihydro-2,7-naphthyridin-1-one (30.00 mg, 0.076 mmol, 1.00 equiv) and 2-(2,6-dioxopiperidin-3-yl)-1,3-dioxo-2,3-dihydro-1H-isoindole-4-carbaldehyde (21.77 mg, 0.076 mmol, 1.00 equiv) in MeOH (2.00 mL) was added AcOH (0.05 mg, 0.001 mmol, 0.01 equiv). The mixture was stirred for 1 hour. NaBH₃CN (9.56 mg, 0.152 mmol, 2.00 equiv) was added. The resulting mixture was stirred for 1 hour. The crude product was purified by preparative HPLC (condition: SunFire C₁₈ OBD Prep Column, 100 Å, 5 μm, 19 mm×250 mm; Mobile Phase A: Water (0.05% TFA), Mobile Phase B: ACN; Flow rate: 25 mL/minuteute; Gradient: 5% B to 5% B in 2 minutes; 254 nm; Rₜ: 12.63 minutes. This afforded 4-[[(1-[[2,6-dimethoxy-4-(2-methyl-1-oxo-1,2-dihydro-2,7-naphthyridin-4-yl)phenyl]methyl]azetidin-3-yl)(methyl)amino]methyl]-2-(2,6-dioxopiperidin-3-yl)-2,3-dihydro-1H-isoindole-1,3-dione (18.90 mg, 0.028 mmol, 36.53%) as a light yellow solid. ¹H NMR (400 MHz, Methanol-d4) δ 9.60 (s, 1H), 8.70 (d, J=6.3 Hz, 1H), 8.00 (s, 1H), 7.96-7.82 (m, 4H), 6.88 (s, 2H), 5.17 (dd, J=12.4, 5.4 Hz, 1H), 4.58 (s, 2H), 4.33 (t, J=7.2 Hz, 4H), 4.10 (d, J=13.2 Hz, 1H), 4.02 (d, J=13.2 Hz, 1H), 3.97 (s, 6H), 3.75 (s, 4H), 2.95-2.83 (m, 1H), 2.81-2.67 (m, 2H), 2.29 (s, 3H), 2.21-2.11 (m, 1H). LCMS (ESI) m/z: [M+H]⁺=665.30.

### Example 35—Preparation of 1-(2,6-dimethoxy-4-(2-methyl-1-oxo-1,2-dihydro-2,7-naphthyridin-4-yl)benzyl)-N-((2-(2,6-dioxopiperidin-3-yl)-1,3-dioxoisoindolin-4-yl)methyl)-N-methylazetidine-3-carboxamide (compound D29)

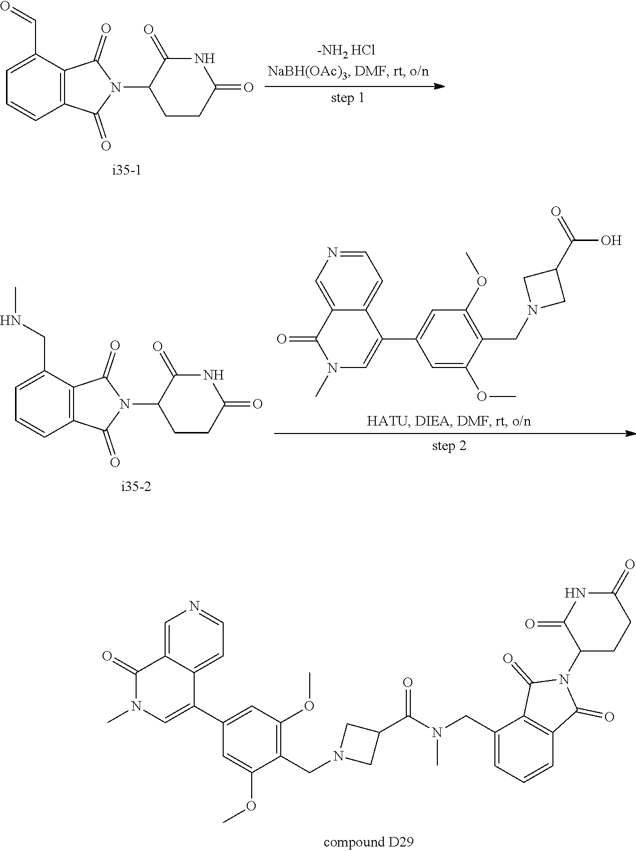 [SMILES: O=Cc1cccc2c1C(=O)N(C1CCC(=O)NC1=O)C2=O ; CNCc1cccc2c1C(=O)N(C1CCC(=O)NC1=O)C2=O ; COc1cc(-c2cn(C)c(=O)c3cnccc23)cc(OC)c1CN1CC(C(=O)O)C1 ; COc1cc(-c2cn(C)c(=O)c3cnccc23)cc(OC)c1CN1CC(C(=O)N(C)Cc2cccc3c2C(=O)N(C2CCC(=O)NC2=O)C3=O)C1]

### Step 1: Preparation of 2-(2,6-dioxopiperidin-3-yl)-4-((methylamino)methyl)isoindoline-1,3-dione (i35-2)

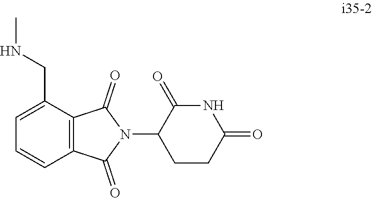 [SMILES: CNCc1cccc2c1C(=O)N(C1CCC(=O)NC1=O)C2=O]

To a solution of 2-(2,6-dioxopiperidin-3-yl)-1,3-dioxoisoindoline-4-carbaldehyde (70.00 mg, 0.245 mmol, 1.00 equiv) in DMF (3.00 mL) was added methanamine hydrochloride (24.77 mg, 0.367 mmol, 1.50 equiv). The resulting mixture was stirred overnight at room temperature. Then NaBH(OAc)₃ (103.88 mg, 0.490 mmol, 2.00 equiv) was added. The resulting mixture was stirred for 1 hour at room temperature. The resulting mixture was purified by reverse phase column with ACN in water from (0% to 50%) to afford 2-(2,6-dioxopiperidin-3-yl)-4-((methylamino)methyl)isoindoline-1,3-dione (30 mg, 41.10%) as a white solid. LCMS (ESI) m/z: [M+H]⁺=302.

### Step 2: Preparation of 1-(2,6-dimethoxy-4-(2-methyl-1-oxo-1,2-dihydro-2,7-naphthyridin-4-yl)benzyl)-N-((2-(2,6-dioxopiperidin-3-yl)-1,3-dioxoisoindolin-4-yl)methyl)-N-methylazetidine-3-carboxamide (compound D29)

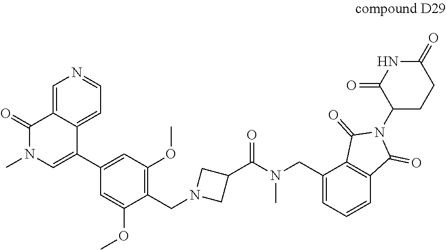 [SMILES: COc1cc(-c2cn(C)c(=O)c3cnccc23)cc(OC)c1CN1CC(C(=O)N(C)Cc2cccc3c2C(=O)N(C2CCC(=O)NC2=O)C3=O)C1]

To a mixture of 1-(2,6-dimethoxy-4-(2-methyl-1-oxo-1,2-dihydro-2,7-naphthyridin-4-yl)benzyl)azetidine-3-carboxylic acid (40.77 mg, 0.100 mmol, 1.00 equiv) in DMF (3.00 mL) was added HATU (94.65 mg, 0.250 mmol, 2.50 equiv) and DIEA (38.61 mg, 0.300 mmol, 3.00 equiv). The resulting mixture was stirred for 2 hours at room temperature. Then 2-(2,6-dioxopiperidin-3-yl)-4-((methylamino)methyl)isoindoline-1,3-dione (30.00 mg, 0.100 mmol, 1.00 equiv) was added. The resulting mixture was stirred for 1 hour. The crude product was purified by preparative HPLC (conditions: XSelect CSH Prep C18 OBD Column, 5 μm, 19*150 mm; Mobile Phase A: Water (0.1% FA), Mobile Phase B: ACN; Flow rate: 25 mL/minuteute; Gradient: 12% B to 12% B in 2 minutes; 254/220 nm; Rₜ: 13.57 min Fractions containing the desired compound were evaporated to dryness to afford 1-(2,6-dimethoxy-4-(2-methyl-1-oxo-1,2-dihydro-2,7-naphthyridin-4-yl)benzyl)-N-((2-(2,6-dioxopiperidin-3-yl)-1,3-dioxoisoindolin-4-yl)methyl)-N-methylazetidine-3-carboxamide (17.10 mg, 24.25%) as a light yellow solid. ¹H-NMR (400 MHz, Methanol-d4) δ 9.58 (s, 1H), 8.69 (t, J=7.8 Hz, 1H), 7.98-7.87 (m, 2H), 7.85-7.77 (m, 2H), 7.72-7.64 (m, 1H), 6.89 (d, J=8.2 Hz, 2H), 5.22-5.01 (m, 3H), 4.65-4.36 (m, 5H), 4.34-4.21 (m, 1H), 4.20-4.07 (m, 1H), 4.01-3.92 (m, 6H), 3.74 (s, 3H), 3.02 (s, 3H), 2.96-2.84 (m, 1H), 2.80-2.71 (m, 2H), 2.24-2.12 (m, 1H). LCMS (ESI) m/z: [M+H]⁺=693.35.

### Example 36—Preparation of 1-(2,6-dimethoxy-4-(2-methyl-1-oxo-1,2-dihydro-2,7-naphthyridin-4-yl)benzyl)-N-(2-((2-((2-(2,6-dioxopiperidin-3-yl)-1,3-dioxoisoindolin-4-yl)amino)ethyl)sulfonyl)ethyl)azetidine-3-carboxamide (compound D30)

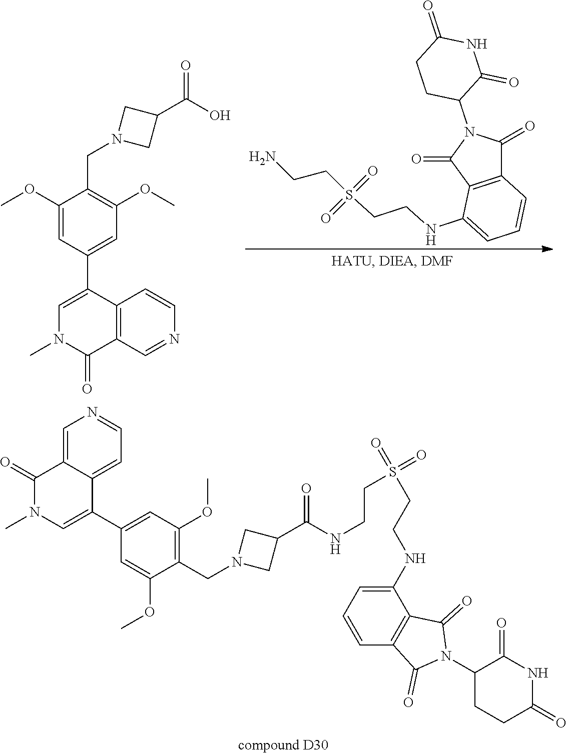

Into a stirred mixture of 1-[[2,6-dimethoxy-4-(2-methyl-1-oxo-2,7-naphthyridin-4-yl)phenyl]methyl]azetidine-3-carboxylic acid (53.00 mg, 0.129 mmol, 1.00 equiv) and DIEA (N,N-diisopropylamine) (50.19 mg, 0.388 mmol, 3.00 equiv) in DMF (dimethylformamide) (1.00 mL) was added 1-[Bis(dimethylamino)methylene]-1H-1,2,3-triazolo[4,5-b]pyridinium 3-oxid hexafluorophosphate, N-[(Dimethylamino)-1H-1,2,3-triazolo-[4,5-b]pyridin-1-ylmethylene]-N-methylmethanaminium hexafluorophosphate N-oxide (HATU) (73.83 mg, 0.194 mmol, 1.50 equiv) at 0° C. After 10 minutes, to the above mixture was added 4-[[2-(2-aminoethanesulfonyl) ethyl]amino]-2-(2,6-dioxopiperidin-3-yl)isoindole-1,3-dione (63.44 mg, 0.155 mmol, 1.20 equiv). Then the reaction was stirred at room temperature for 2 hours under N₂ atmosphere. The crude product was purified by Prep-HPLC (conditions: Sunfire C18 OBD Prep Column, 5 μm, 19 mm*250 mm; Mobile Phase A: Water (0.05% TFA, trifluoroacetic acid), Mobile Phase B: acetonitrile (MeCN or ACN); Flow rate: 25 mL/minuteute; Gradient: 3% B to 3% B in 2 minutes; 254 nm; Rₜ: 13.98 minutes). This resulted in 1-(2,6-dimethoxy-4-(2-methyl-1-oxo-1,2-dihydro-2,7-naphthyridin-4-yl)benzyl)-N-(2-((2-((2-(2,6-dioxopiperidin-3-yl)-1,3-dioxoisoindolin-4-yl)amino)ethyl)sulfonyl)ethyl)azetidine-3-carboxamide 18.4 mg (16.47%) as a yellow solid. ¹H NMR (300 MHz, Methanol-d4) δ 9.52 (d, J=0.8 Hz, 1H), 8.69 (d, J=5.8 Hz, 1H), 8.56 (br s, 0.5H, FA), 7.77 (s, 1H), 7.67-7.55 (m, 2H), 7.13 (t, J=7 0.6 Hz, 2H), 6.83 (s, 2H), 5.06 (dd, J=12.3, 5.4 Hz, 1H), 4.37 (s, 2H), 4.23-4.06 (m, 4H), 3.95 (s, 6H), 3.89 (t, J=6.3 Hz, 2H), 3.77-3.69 (m, 2H), 3.71 (s, 3H), 3.52 (q, J=6.9, 6.3 Hz, 3H), 3.38 (t, J=6.3 Hz, 2H), 2.62-2.93 (m, 3H), 2.07-2.17 (m, 1H). LCMS (ESI) m/z: [M+H]⁺=800.35.

### Example 37—Preparation of 5-((1-(3-((2,6-dimethoxy-4-(2-methyl-1-oxo-1,2-dihydro-2,7-naphthyridin-4-yl)benzyl)amino)propyl)azetidin-3-yl)oxy)-2-(2,6-dioxopiperidin-3-yl)isoindoline-1,3-dione formic acid (compound D31 formic acid)

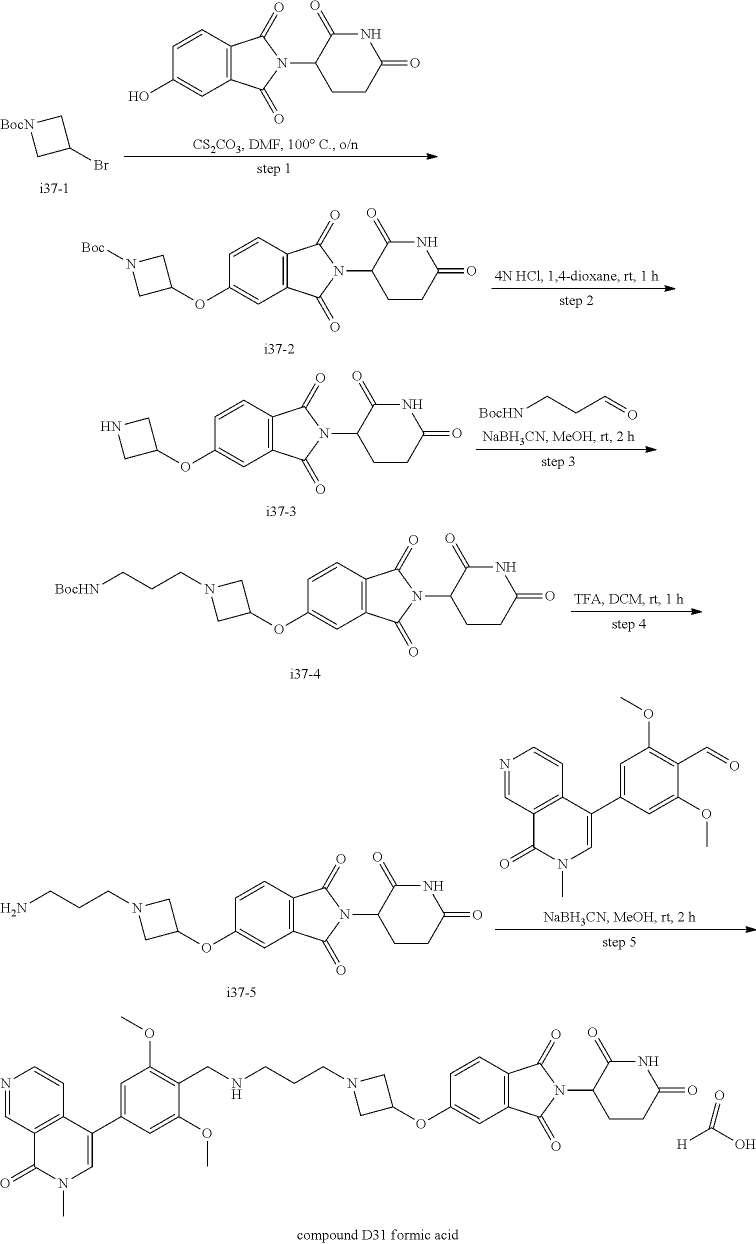

### Step 1: Preparation of tert-butyl 3-((2-(2,6-dioxopiperidin-3-yl)-1,3-dioxoisoindolin-5-yl)oxy)azetidine-1-carboxylate (i37-2)

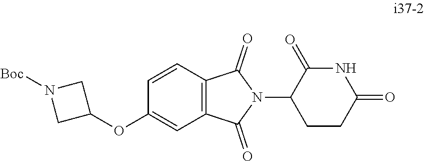 [SMILES: CC(C)(C)OC(=O)N1CC(Oc2ccc3c(c2)C(=O)N(C2CCC(=O)NC2=O)C3=O)C1]

To a mixture of tert-butyl 3-bromoazetidine-1-carboxylate (2.00 g, 8.511 mmol, 1.00 equiv) and 2-(2,6-dioxopiperidin-3-yl)-5-hydroxyisoindoline-1,3-dione (2.33 g, 8.511 mmol, 1.00 equiv) in DMF (30.00 mL) was added Cs₂CO₃ (5.53 g, 17.022 mmol, 2.00 equiv). The resulting mixture was stirred overnight at 90° C. The resulting mixture was concentrated under vacuum. The residue was purified by silica gel column chromatography, eluted with EA in PE from 0% to 50% to afford tert-butyl 3-((2-(2,6-dioxopiperidin-3-yl)-1,3-dioxoisoindolin-5-yl)oxy)azetidine-1-carboxylate (400 mg, 10.96%) as a light yellow solid. LCMS (ESI) m/z: [M+H]+=430.

### Step 2: Preparation of 5-(azetidin-3-yloxy)-2-(2,6-dioxopiperidin-3-yl)isoindoline-1,3-dione (i37-3)

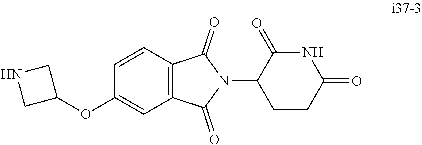 [SMILES: O=C1CCC(N2C(=O)c3ccc(OC4CNC4)cc3C2=O)C(=O)N1]

To a solution of tert-butyl 3-((2-(2,6-dioxopiperidin-3-yl)-1,3-dioxoisoindolin-5-yl)oxy)azetidine-1-carboxylate (400.00 mg, 0.932 mmol, 1.00 equiv) in 1,4-dioxane (5 mL) was added HCl (4 N in 1,4-dioxane) (5 mL, 20.000 mmol, 21.46 equiv). The resulting solution was stirred for 1 hour at room temperature. The resulting mixture was concentrated under vacuum to afford 5-(azetidin-3-yloxy)-2-(2,6-dioxopiperidin-3-yl)isoindoline-1,3-dione (440.00 mg, crude) as a white solid. LCMS (ESI) m/z: [M+H]⁺=330.

### Step 3: Preparation of tert-butyl (3-(3-((2-(2,6-dioxopiperidin-3-yl)-1,3-dioxoisoindolin-5-yl)oxy) zetidin-1-yl)propyl)carbamate (i37-4)

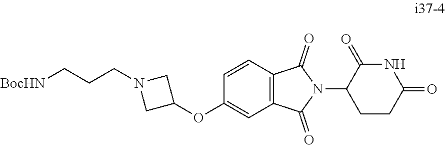 [SMILES: CC(C)(C)OC(=O)NCCCN1CC(Oc2ccc3c(c2)C(=O)N(C2CCC(=O)NC2=O)C3=O)C1]

A mixture of 5-(azetidin-3-yloxy)-2-(2,6-dioxopiperidin-3-yl)isoindoline-1,3-dione (200.00 mg, 0.608 mmol, 1.00 equiv) and tert-butyl (3-oxopropyl)carbamate (i05.18 mg, 0.608 mmol, 1.00 equiv) in MeOH (5.00 mL) was stirred for 1.5 hours at room temperature. Then NaBH₃CN (75.39 mg, 1.216 mmol, 2.00 equiv) was added. The resulting mixture was stirred for 1 hour at room temperature. The resulting mixture was concentrated under vacuum. The residue was purified by silica gel column chromatography, eluted with EA in PE from 0% to 50% to afford tert-butyl (3-(3-((2-(2,6-dioxopiperidin-3-yl)-1,3-dioxoisoindolin-5-yl)oxy)zetidin-1-yl)propyl)carbamate (i00.00 mg, 33.89%) as a white solid. LCMS (ESI) m/z: [M+H]⁺=487.

### Step 4: Preparation of 5-((1-(3-aminopropyl)azetidin-3-yl)oxy)-2-(2,6-dioxopiperidin-3-yl) isoindoline-1,3-dione (i37-5)

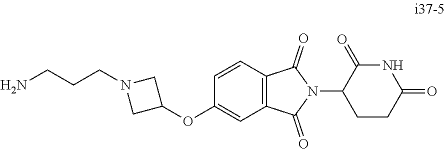 [SMILES: NCCCN1CC(Oc2ccc3c(c2)C(=O)N(C2CCC(=O)NC2=O)C3=O)C1]

To a solution of tert-butyl (3-(3-((2-(2,6-dioxopiperidin-3-yl)-1,3-dioxoisoindolin-5-yl)oxy) azetidin-1-yl)propyl)carbamate (i00.00 mg, 0.206 mmol, 1.00 equiv) in DCM (4.00 mL) was added TFA (4.00 mL, 53.860 mmol, 261.46 equiv). The resulting mixture was stirred for one hour at room temperature. The resulting mixture was concentrated under vacuum to afford 5-((1-(3-aminopropyl)azetidin-3-yl)oxy)-2-(2,6-dioxopiperidin-3-yl)isoindoline-1,3-dione(120 mg, crude). LCMS (ESI) m/z: [M+H]+=387.

### Step 5: Preparation of 5-((1-(3-((2,6-dimethoxy-4-(2-methyl-1-oxo-1,2-dihydro-2,7-naphthyridin-4-yl)benzyl)amino)propyl)azetidin-3-yl)oxy)-2-(2,6-dioxopiperidin-3-yl)isoindoline-1,3-dione formic acid (compound D31 formic acid)

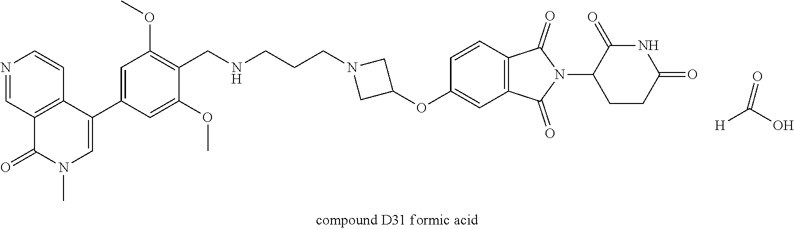 [SMILES: COc1cc(-c2cn(C)c(=O)c3cnccc23)cc(OC)c1CNCCCN1CC(Oc2ccc3c(c2)C(=O)N(C2CCC(=O)NC2=O)C3=O)C1 ; O=CO]

To a solution of 5-((1-(3-aminopropyl)azetidin-3-yl)oxy)-2-(2,6-dioxopiperidin-3-yl)isoindoline-1,3-dione (60.00 mg, 0.155 mmol, 1.00 equiv) in MeOH (5.00 mL, 123.495 mmol, 795.32 equiv) was added 2,6-dimethoxy-4-(2-methyl-1-oxo-2,7-naphthyridin-4-yl)benzaldehyde (50.36 mg, 0.155 mmol, 1 equiv). The resulting mixture was stirred for 1 hour. Then NaBH₃CN (19.52 mg, 0.311 mmol, 2 equiv) was added. The resulting mixture was stirred for 1 hour. The resulting mixture was filtered, and the filtrate was purified by prep-HPLC (conditions: SunFire C18 OBD Prep Column, 100 Å, 5 μm, 19 mm×250 mm; Mobile Phase A: Water (0.1% FA), Mobile Phase B: ACN; Flow rate: 25 mL/minute; Gradient: 5% B to 5% B in 2 minutes; 254 nm; Rt: 9.75 minutes) to afford 5-((1-(3-((2,6-dimethoxy-4-(2-methyl-1-oxo-1,2-dihydro-2,7-naphthyridin-4-yl)benzyl)amino)propyl)azetidin-3-yl)oxy)-2-(2,6-dioxopiperidin-3-yl)isoindoline-1,3-dione; formate (14.4 mg, 12.52%) as a yellow solid. ¹H NMR (400 MHz, Methanol-d4) δ 9.52 (s, 1H), 8.67 (d, J=5.7 Hz, 1H), 8.26 (br s, 0.65H, FA), 7.82-7.75 (m, 2H), 7.60 (dd, J=5.8, 0.9 Hz, 1H), 7.21 (dq, J=4.6, 2.3 Hz, 2H), 6.88 (s, 2H), 5.13-5.00 (m, 2H), 4.36 (s, 2H), 3.99 (s, 6H), 3.93-3.89 (m, 2H), 3.71 (s, 3H), 3.44 (d, J=8.2 Hz, 2H), 3.22 (t, J=6.7 Hz, 2H), 2.95-2.82 (m, 3H), 2.82-2.62 (m, 2H), 2.21-2.05 (m, 1H), 1.89-1.81 (m, 2H). LCMS (ESI) m/z: [M+H]⁺=695.40.

### Example 38—Preparation of 4-(4-(6-(2,6-dimethoxy-4-(2-methyl-1-oxo-1,2-dihydro-2,7-naphthyridin-4-yl)benzyl)-2,6-diazaspiro[3.3]heptan-2-yl)-4-oxobutoxy)-2-(2,6-dioxopiperidin-3-yl)isoindoline-1,3-dione (compound D32)

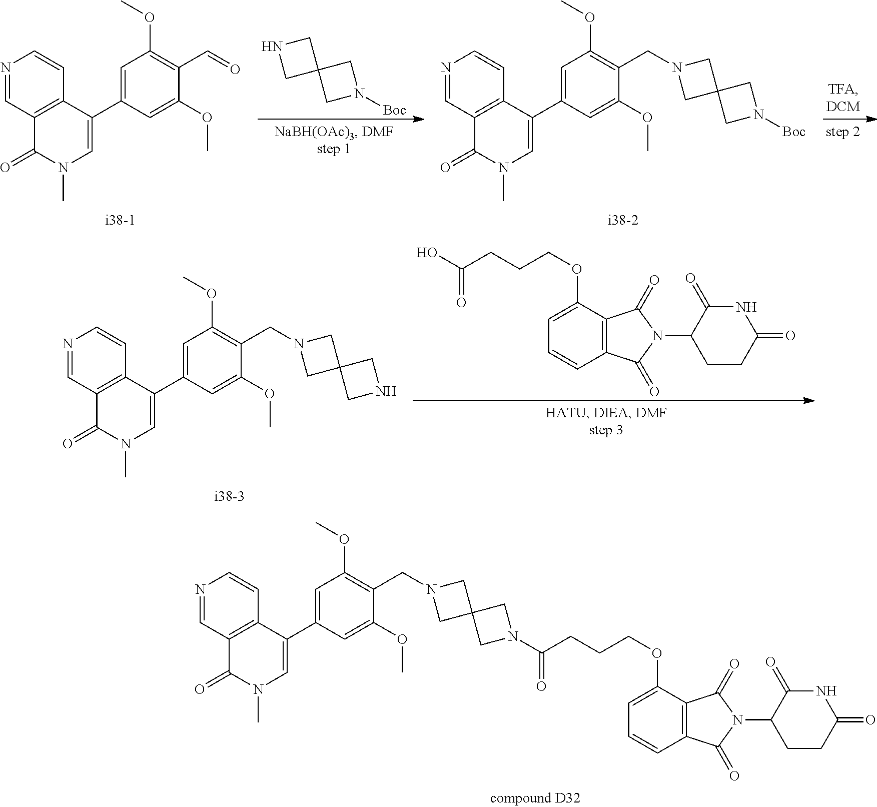

### Step 1: Preparation of tert-butyl 6-(2,6-dimethoxy-4-(2-methyl-1-oxo-1,2-dihydro-2,7-naphthyridin-4-yl)benzyl)-2,6-diazaspiro[3.3]heptane-2-carboxylate (i38-2)

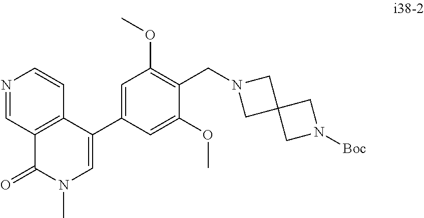 [SMILES: COc1cc(-c2cn(C)c(=O)c3cnccc23)cc(OC)c1CN1CC2(C1)CN(C(=O)OC(C)(C)C)C2]

To a solution of 2,6-dimethoxy-4-(2-methyl-1-oxo-2,7-naphthyridin-4-yl)benzaldehyde(700.00 mg, 2.158 mmol, 1.00 equiv) and tert-butyl 2,6-diazaspiro[3.3]heptane-2-carboxylate (427.91 mg, 2.158 mmol, 1.00 equiv) in DMF (10.00 mL, 129.218 mmol, 59.87 equiv) was added NaBH(OAc)₃ (914.85 mg, 4.317 mmol, 2.00 equiv). The resulting solution was stirred at 25° C. for 1 hour. The mixture was concentrated to give crude product that was purified by chromatography on silica gel eluted with MeOH/DCM (6:94) to give tert-butyl 6-[[2,6-dimethoxy-4-(2-methyl-1-oxo-2,7-naphthyridin-4-yl)phenyl]methyl]-2,6-diazaspiro [3.3]heptane-2-carboxylate(808 mg, 73.90%) as an off-white solid. LCMS (ESI) m/z: [M+H]⁺=507.

### Step 2: Preparation of 4-(4-((2,6-diazaspiro[3.3]heptan-2-yl)methyl)-3,5-dimethoxyphenyl)-2-methyl-2,7-naphthyridin-1(2H)-one (i38-3)

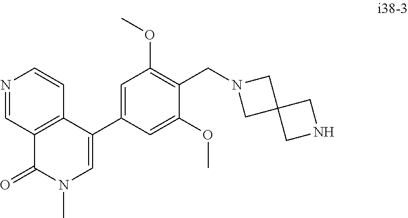 [SMILES: COc1cc(-c2cn(C)c(=O)c3cnccc23)cc(OC)c1CN1CC2(CNC2)C1]

A solution of tert-butyl 6-[[2,6-dimethoxy-4-(2-methyl-1-oxo-2,7-naphthyridin-4-yl)phenyl]methyl]-2,6-diazaspiro[3.3]heptane-2-carboxylate (708.00 mg, 1.398 mmol, 1.00 equiv) and TFA (1.50 mL, 20.195 mmol, 14.45 equiv) in DCM (7.00 mL, 110.110 mmol, 78.79 equiv) was stirred at 25° C. for 1 hour. The mixture was concentrated to give crude product 4-(4-[2, 6-diazaspiro[3.3]heptan-2-ylmethyl]-3,5-dimethoxyphenyl)-2-methyl-2,7-naphthyridin-1-one(696 mg) as a brown oil that was used directly without further purification. LCMS (ESI) m/z: [M+H]+=407.

### Step 3: Preparation of 4-(4-(6-(2,6-dimethoxy-4-(2-methyl-1-oxo-1,2-dihydro-2,7-naphthyridin-4-yl)benzyl)-2,6-diazaspiro[3.3]heptan-2-yl)-4-oxobutoxy)-2-(2,6-dioxopiperidin-3-yl)isoindoline-1,3-dione (compound D32)

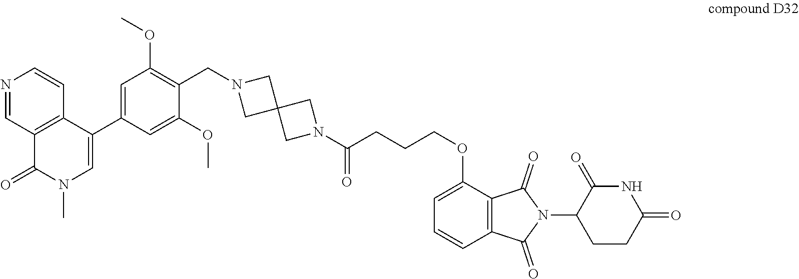 [SMILES: COc1cc(-c2cn(C)c(=O)c3cnccc23)cc(OC)c1CN1CC2(C1)CN(C(=O)CCCOc1cccc3c1C(=O)N(C1CCC(=O)NC1=O)C3=O)C2]

To a solution of 4-(4-[2,6-diazaspiro[3.3]heptan-2-ylmethyl]-3,5-dimethoxyphenyl)-2-methyl-2,7-naphthyridin-1-one (40.00 mg, 0.098 mmol, 1.00 equiv) and 4-[[2-(2,6-dioxopiperidin-3-yl)-1,3-dioxoisoindol-4-yl]oxy]butanoic acid (35.46 mg, 0.098 mmol, 1.00 equiv) in DMF (1.0 mL) was added HATU (56.12 mg, 0.148 mmol, 1.5 equiv) and DIEA (31.80 mg, 0.246 mmol, 10 equiv). The mixture was stirred at 25° C. for 1 hour. The mixture was purified by prep-HPLC (conditions: Kinetex EVO C₁₈ Column 21.2*150, 5 μm; Mobile Phase A: Water (0.1% FA), Mobile Phase B: ACN; Flow rate: 25 mL/minute; Gradient: 16% B to 26% B in 8 minutes; 254/220 nm; Rₜ: 7.03 minutes) to afford 4-[4-(6-[[2,6-dimethoxy-4-(2-methyl-1-oxo-2,7-naphthyridin-4-yl)phenyl]methyl]-2,6-diazaspiro[3.3]heptan-2-yl)-4-oxobutoxy]-2-(2,6-dioxopiperidin-3-yl)isoindole-1,3-dione (12 mg, 16.29%) as a white solid. ¹H NMR (400 MHz, Methanol-d4) δ 9.53 (s, 1H), 8.70 (d, J=5.8 Hz, 1H), 7.79 (dd, J=8.5, 7.4 Hz, 1H), 7.78 (s, 1H), 7.62 (d, J=5.8 Hz, 1H), 7.46 (dd, J=14.7, 7.9 Hz, 2H), 6.86 (s, 2H), 5.13 (dd, J=12.5, 5.4 Hz, 1H), 4.60 (s, 1H), 4.40 (d, J=13.7 Hz, 4H), 4.32-4.19 (m, 6H), 4.14 (s, 2H), 3.96 (s, 6H), 3.71 (s, 3H), 2.95-2.68 (m, 3H), 2.53-2.34 (m, 2H), 2.20-2.10 (m, 3H). LCMS (ESI) m/z: [M+H]⁺=749.40.

### Example 39—Preparation of 4-(4-(4-(2,6-dimethoxy-4-(2-methyl-1-oxo-1,2-dihydro-2,7-naphthyridin-4-yl)benzyl) piperazin-1-yl)-4-oxobutoxy)-2-(2,6-dioxopiperidin-3-yl)isoindoline-1,3-dione (compound D33)

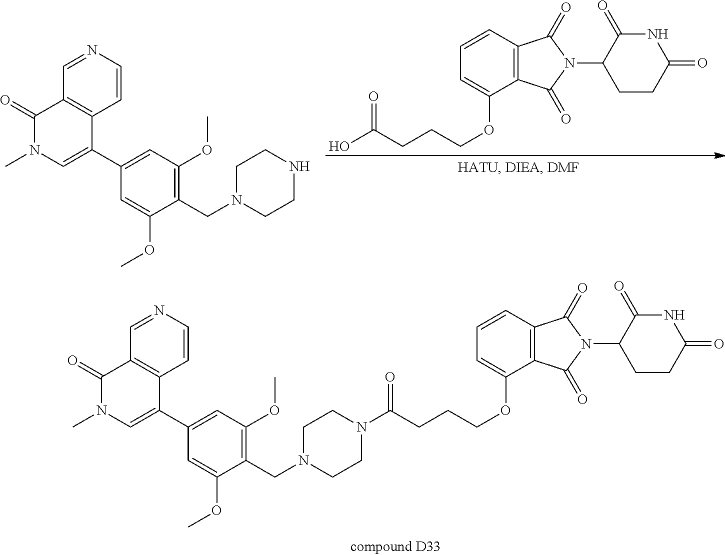 [SMILES: COc1cc(-c2cn(C)c(=O)c3cnccc23)cc(OC)c1CN1CCNCC1 ; O=C(O)CCCOc1cccc2c1C(=O)N(C1CCC(=O)NC1=O)C2=O ; COc1cc(-c2cn(C)c(=O)c3cnccc23)cc(OC)c1CN1CCN(C(=O)CCCOc2cccc3c2C(=O)N(C2CCC(=O)NC2=O)C3=O)CC1]

To a stirred mixture of 4-[3,5-dimethoxy-4-(piperazin-1-ylmethyl)phenyl]-2-methyl-2,7-naphthyridin-1-one (50.00 mg, 0.127 mmol, 1.00 equiv) and 4-[[2-(2,6-dioxopiperidin-3-yl)-1,3-dioxoisoindol-4-yl]oxy]butanoic acid (45.67 mg, 0.127 mmol, 1.00 equiv) in DMF (2.00 mL) was added DIEA (163.82 mg, 1.268 mmol, 10.00 equiv) and HATU (96.39 mg, 0.254 mmol, 2.00 equiv) at 0° C. The above mixture was stirred for 3 hours at room temperature. Then the crude product was purified by preparative HPLC (conditions: XBridge Shield RP18 OBD Column, 5 μm, 19*250 mm; Mobile Phase A: Water (0.1% FA), Mobile Phase B: ACN; Flow rate: 25 mL/minute; Gradient: 12% B to 26% B in 8 minutes; 254 nm; Rₜ: 7.91 minutes). This resulted in 4-(4-(4-(2,6-dimethoxy-4-(2-methyl-1-oxo-1,2-dihydro-2,7-naphthyridin-4-yl)benzyl)piperazin-1-yl)-4-oxobutoxy)-2-(2,6-dioxopiperidin-3-yl)isoindoline-1,3-dione (5.60 mg, 5.54%) as a white solid. ¹H NMR (300 MHz, Methanol-d4) δ 9.54 (s, 1H), 8.69 (d, J=5.8 Hz, 1H), 7.85-7.73 (m, 2H), 7.63 (d, J=5.7 Hz, 1H), 7.52-7.43 (m, 2H), 6.79 (s, 2H), 5.11 (dd, J=12.2, 5.4 Hz, 1H), 4.30 (t, J=5.8 Hz, 2H), 4.01 (s, 2H), 3.90 (s, 6H), 3.81-3.65 (m, 7H), 2.98-2.81 (m, 6H), 2.79-2.67 (m, 3H), 2.24-2.07 (m, 3H). vLCMS (ESI) m/z: [M+H]⁺=737.40.

### Example 40—Preparation of 4-((5-(6-(2,6-dimethoxy-4-(2-methyl-1-oxo-1,2-dihydro-2,7-naphthyridin-4-yl)benzyl)-2,6-diazaspiro[3.3]heptan-2-yl)-5-oxopentyl)oxy)-2-(2,6-dioxopiperidin-3-yl)isoindoline-1,3-dione (compound D34)

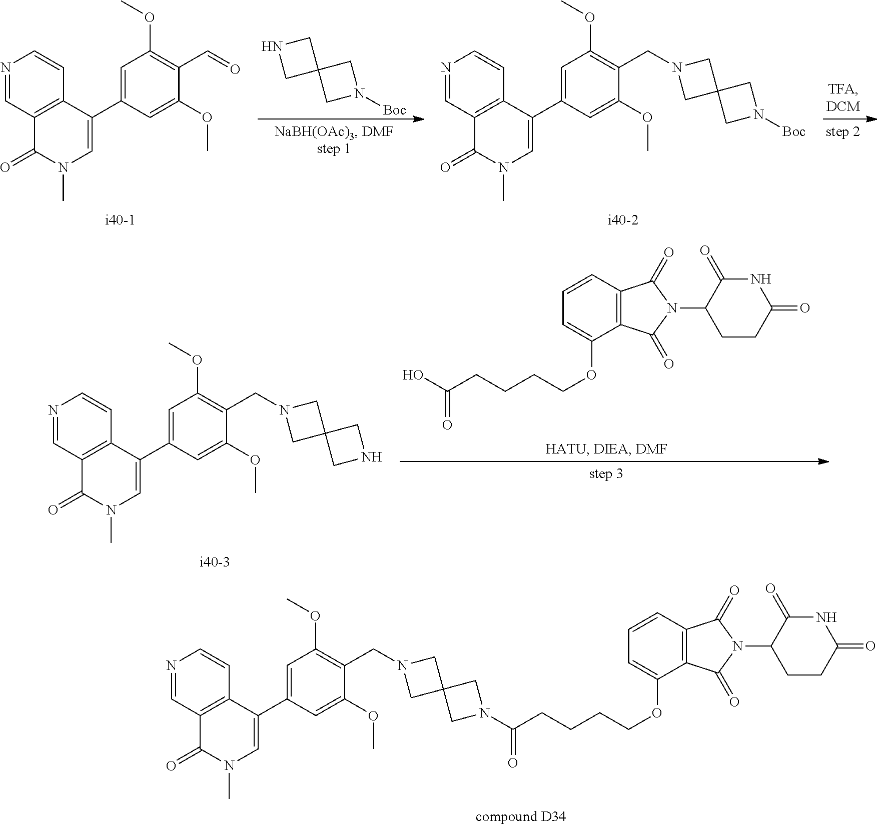

### Step 1: Preparation of tert-butyl 6-(2,6-dimethoxy-4-(2-methyl-1-oxo-1,2-dihydro-2,7-naphthyridin-4-yl)benzyl)-2,6-diazaspiro[3.3]heptane-2-carboxylate (i40-2)

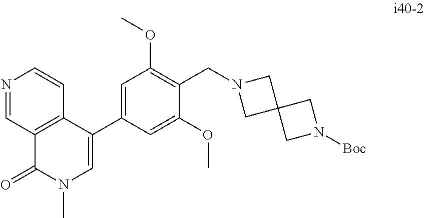 [SMILES: COc1cc(-c2cn(C)c(=O)c3cnccc23)cc(OC)c1CN1CC2(C1)CN(C(=O)OC(C)(C)C)C2]

To a solution of 2,6-dimethoxy-4-(2-methyl-1-oxo-2,7-naphthyridin-4-yl)benzaldehyde (700.00 mg, 2.158 mmol, 1.00 equiv) and tert-butyl 2,6-diazaspiro[3.3]heptane-2-carboxylate (427.91 mg, 2.158 mmol, 1.00 equiv) in DMF (10.00 mL, 129.218 mmol, 59.87 equiv) was added NaBH(OAc)₃ (914.85 mg, 4.317 mmol, 2.00 equiv). The resulting solution was stirred at 25° C. for 1 hour. The mixture was concentrated to give crude product that was purified by chromatography on silica gel eluted with MeOH]/DCM (6:94) to give tert-butyl 6-[[2,6-dimethoxy-4-(2-methyl-1-oxo-2,7-naphthyridin-4-yl)phenyl]methyl]-2,6-diazaspiro[3.3]heptane-2-carboxylate(808 mg, 73.90%) as an off-white solid. LCMS (ESI) m/z: [M+H]+=507.

### Step 2: Preparation of 4-(4-((2,6-diazaspiro[3.3]heptan-2-yl)methyl)-3,5-dimethoxyphenyl)-2-methyl-2,7-naphthyridin-1(2H)-one (i40-3)

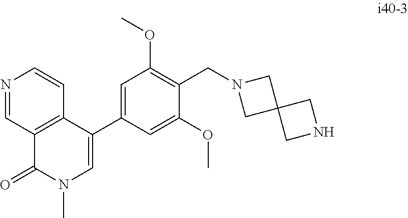 [SMILES: COc1cc(-c2cn(C)c(=O)c3cnccc23)cc(OC)c1CN1CC2(CNC2)C1]

A solution of tert-butyl 6-[[2,6-dimethoxy-4-(2-methyl-1-oxo-2,7-naphthyridin-4-yl)phenyl]methyl]-2,6-diazaspiro[3.3]heptane-2-carboxylate (708.00 mg, 1.398 mmol, 1.00 equiv) and TFA (1.50 mL, 20.195 mmol, 14.45 equiv) in DCM (7 mL) was stirred at 25° C. for 1 hour. The mixture was concentrated to give crude product 4-(4-[2,6-diazaspiro[3.3]heptan-2-ylmethyl]-3,5-dimethoxyphenyl)-2-methyl-2,7-naphthyridin-1-one (696 mg) as a brown oil that was used directly without further purification. LCMS (ESI) m/z: [M+H]+=407.

### Step 3: Preparation of 4-((5-(6-(2,6-dimethoxy-4-(2-methyl-1-oxo-1,2-dihydro-2,7-naphthyridin-4-yl)benzyl)-2,6-diazaspiro[3.3]heptan-2-yl)-5-oxopentyl)oxy)-2-(2,6-dioxopiperidin-3-yl)isoindoline-1,3-dione (compound D34)

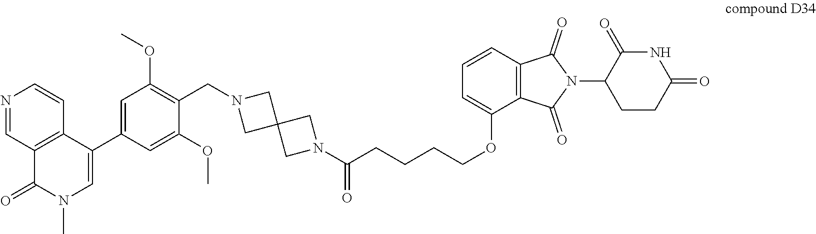 [SMILES: COc1cc(-c2cn(C)c(=O)c3cnccc23)cc(OC)c1CN1CC2(C1)CN(C(=O)CCCCOc1cccc3c1C(=O)N(C1CCC(=O)NC1=O)C3=O)C2]

To a solution of 4-(4-[2,6-diazaspiro[3.3]heptan-2-ylmethyl]-3,5-dimethoxyphenyl)-2-methyl-2,7-naphthyridin-1-one (40.00 mg, 0.098 mmol, 1.00 equiv) and 5-[[2-(2,6-dioxopiperidin-3-yl)-1,3-dioxoisoindol-4-yl]oxy]pentanoic acid (36.84 mg, 0.098 mmol, 1 equiv) in DMF (1 mL) was added HATU (56.12 mg, 0.148 mmol, 1.5 equiv) and DIEA (31.80 mg, 0.246 mmol, 10 equiv). The mixture was stirred at 25° C. for 1 hour. The mixture was purified by prep-HPLC (conditions: XSelect CSH Prep C18 OBD Column, 5 μm, 19*150 mm; Mobile Phase A: Water (0.1% FA), Mobile Phase B: ACN; Flow rate: 25 mL/minute; Gradient: 12% B to 22% B in 12 minutes; 254/220 nm; Rt: 10.52 minutes) to afford 4-[[5-(6-[[2,6-dimethoxy-4-(2-methyl-1-oxo-2,7-naphthyridin-4-yl)phenyl]methyl]-2,6-diazaspiro[3.3]heptan-2-yl)-5-oxop entyl]oxy]-2-(2,6-dioxopiperidin-3-yl)isoindole-1,3-dione (10.1 mg, 13.46%) as a light yellow solid. ¹H NMR (400 MHz, Methanol-d4) δ 9.58 (s, 1H), 8.73-8.67 (m, 1H), 7.92 (d, J=6.9 Hz, 1H), 7.84-7.76 (m, 1H), 7.47 (t, J=8.1 Hz, 2H), 6.89 (d, J=3.5 Hz, 2H), 5.17-5.07 (m, 1H), 4.51 (d, J=3.0 Hz, 2H), 4.45-4.31 (m, 6H), 4.27 (t, J=5.5 Hz, 2H), 4.19 (s, 1H), 4.12 (s, 1H), 3.98 (d, J=3.4 Hz, 6H), 3.74 (d, J=1.7 Hz, 3H), 2.96-2.65 (m, 3H), 2.34-2.30 (m, 2H), 2.19-2.12 (m, 1H), 1.96-1.89 (m, 2H), 1.88-1.80 (m, 2H). LCMS (ESI) m/z: [M+H]⁺=763.40.

### Example 41—Preparation of 4-((5-(4-(2,6-dimethoxy-4-(2-methyl-1-oxo-1,2-dihydro-2,7-naphthyridin-4-yl)benzyl)piperazin-1-yl)-5-oxopentyl)oxy)-2-(2,6-dioxopiperidin-3-yl)isoindoline-1,3-dione formic acid (compound D35 formic acid)

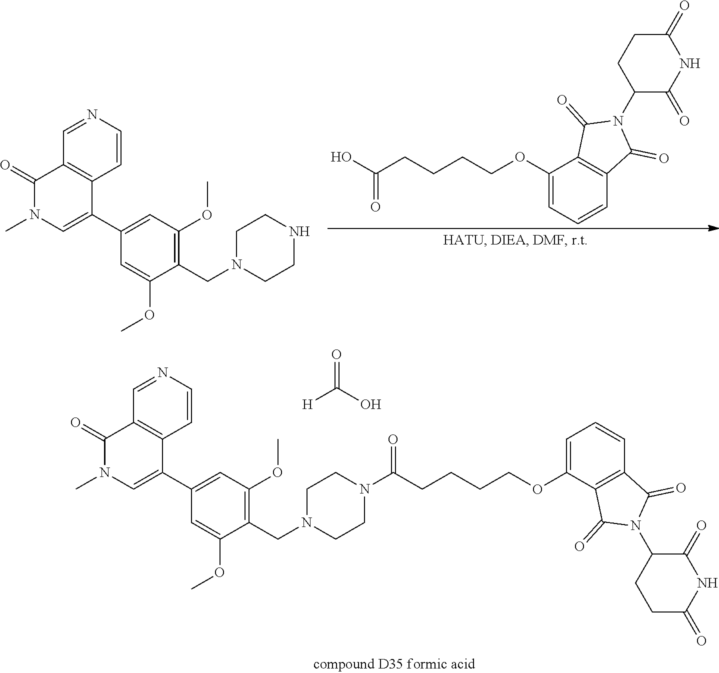

To a stirred solution of 4-[3,5-dimethoxy-4-(piperazin-1-ylmethyl)phenyl]-2-methyl-2,7-naphthyridin-1-one (50.0 mg, 0.127 mmol, 1.00 equiv) and 5-[[2-(2,6-dioxopiperidin-3-yl)-1,3-dioxoisoindol-4-yl]oxy]pentanoic acid (47.5 mg, 0.127 mmol, 1.00 equiv) in DMF (1 mL) was added DIEA (163.8 mg, 1.268 mmol, 10.00 equiv) dropwise at room temperature. The resulting mixture was stirred for 10 min at room temperature. To the above mixture was added HATU (96.4 mg, 0.254 mmol, 2.00 equiv). The resulting mixture was stirred for additional 2 hours at room temperature. The residue was purified by reverse flash chromatography (conditions: SunFire C18 OBD Prep Column, 100 Å, 5 μm, 19 mm×250 mm; Mobile Phase A: Water (0.1% FA), Mobile Phase B: ACN; Flow rate: 25 mL/minute; Gradient: 9 B to 27 B in 10 minutes; 254 nm; R_{T}: 10.12) to afford 4-[[5-(4-[[2,6-dimethoxy-4-(2-methyl-1-oxo-2,7-naphthyridin-4-yl)phenyl]methyl]piperazin-1-yl)-5-oxopentyl]oxy]-2-(2,6-dioxopiperidin-3-yl)isoindole-1,3-dione (6.6 mg, 6.7%) as a white solid. ¹H NMR (400 MHz, Methanol-d4) δ 9.53 (d, J=0.9 Hz, 1H), 8.69 (d, J=5.8 Hz, 1H), 8.45 (br s, 0.13H, FA), 7.81-7.73 (m, 2H), 7.64 (dd, J=5.8, 0.9 Hz, 1H), 7.45 (dd, J=7.9, 6.2 Hz, 2H), 6.79 (s, 2H), 5.11 (dd, J=12.5, 5.5 Hz, 1H), 4.28 (t, J=5.7 Hz, 2H), 3.97 (s, 2H), 3.90 (s, 6H), 3.74-3.62 (m, 7H), 2.95-2.81 (m, 3H), 2.80-2.65 (m, 4H), 2.60 (t, J=7.4 Hz, 2H), 2.17-2.07 (m, 1H), 1.99-1.83 (m, 4H). LCMS (ESI) m/z: [M+H]+=751.40

### Example 42—Preparation of 4-(2-(6-(2,6-dimethoxy-4-(2-methyl-1-oxo-1,2-dihydro-2,7-naphthyridin-4-yl)benzyl)-2,6-diazaspiro[3.3]heptan-2-yl)-2-oxoethoxy)-2-(2,6-dioxopiperidin-3-yl)isoindoline-1,3-dione (compound D36 formic acid)

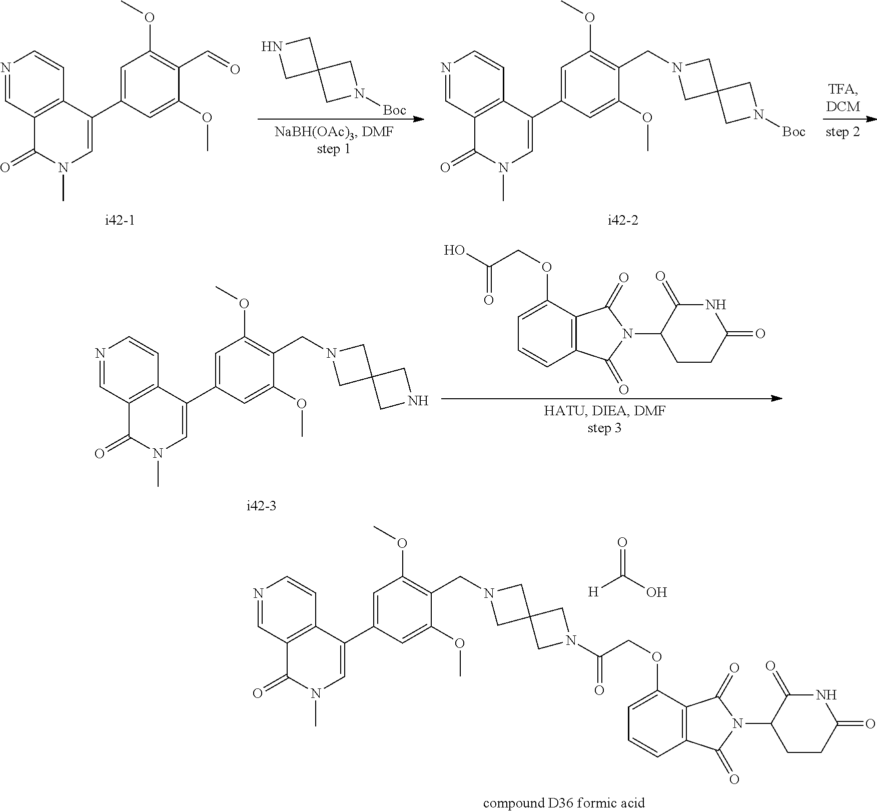

### Step 1: Preparation of tert-butyl 6-(2,6-dimethoxy-4-(2-methyl-1-oxo-1,2-dihydro-2,7-naphthyridin-4-yl)benzyl)-2,6-diazaspiro[3.3]heptane-2-carboxylate (i42-2)

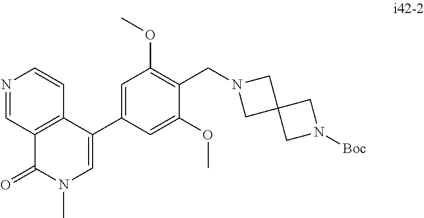 [SMILES: COc1cc(-c2cn(C)c(=O)c3cnccc23)cc(OC)c1CN1CC2(C1)CN(C(=O)OC(C)(C)C)C2]

To a solution of 2,6-dimethoxy-4-(2-methyl-1-oxo-2,7-naphthyridin-4-yl)benzaldehyde (700.00 mg, 2.158 mmol, 1.00 equiv) and tert-butyl 2,6-diazaspiro[3.3]heptane-2-carboxylate (427.91 mg, 2.158 mmol, 1.00 equiv) in DMF (10 mL) was added NaBH(OAc)₃ (914.85 mg, 4.317 mmol, 2.00 equiv). The resulting solution was stirred at 25° C. for 1 hour. The mixture was concentrated to give crude product that was purified by chromatography on silica gel eluted with MeOH]/DCM (6:94) to give tert-butyl 6-[[2,6-dimethoxy-4-(2-methyl-1-oxo-2,7-naphthyridin-4-yl)phenyl]methyl]-2,6-diazaspiro[3.3]heptane-2-carboxylate (808 mg, 73.90%) as an off-white solid. LCMS (ESI) m/z: [M+H]⁺=507.

### Step 2: Preparation of 4-(4-((2,6-diazaspiro[3.3]heptan-2-yl)methyl)-3,5-dimethoxyphenyl)-2-methyl-2,7-naphthyridin-1(2H)-one (i42-3)

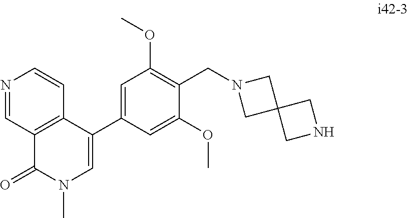 [SMILES: COc1cc(-c2cn(C)c(=O)c3cnccc23)cc(OC)c1CN1CC2(CNC2)C1]

To a solution of tert-butyl 6-[[2,6-dimethoxy-4-(2-methyl-1-oxo-2,7-naphthyridin-4-yl)phenyl]methyl]-2,6-diazaspiro[3.3]heptane-2-carboxylate (708.00 mg, 1.398 mmol, 1.00 equiv) and TFA (1.50 mL, 20.195 mmol, 14.45 equiv) in DCM (7 mL) was stirred at 25° C. for 1 hour. The mixture was concentrated to give crude product 4-(4-[2,6-diazaspiro[3.3]heptan-2-ylmethyl]-3,5-dimethoxyphenyl)-2-methyl-2,7-naphthyridin-1-one(696 mg) as a brown oil that was used directly without further purification. LCMS (ESI) m/z: [M+H]+=407.

### Step 3: Preparation of 4-(2-(6-(2,6-dimethoxy-4-(2-methyl-1-oxo-1,2-dihydro-2,7-naphthyridin-4-yl)benzyl)-2,6-diazaspiro[3.3]heptan-2-yl)-2-oxoethoxy)-2-(2,6-dioxopiperidin-3-yl)isoindoline-1,3-dione formic acid (compound D35 formic acid)

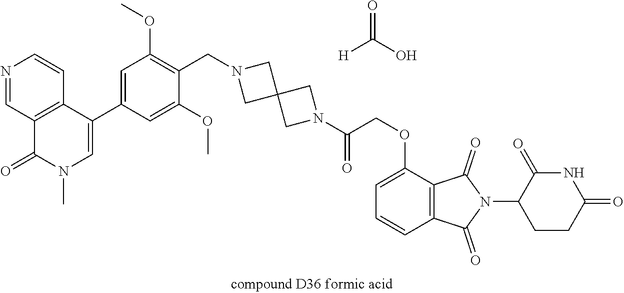

To a solution of 4-(4-[2,6-diazaspiro[3.3]heptan-2-ylmethyl]-3,5-dimethoxyphenyl)-2-methyl-2,7-naphthyridin-1-one (40.00 mg, 0.098 mmol, 1.00 equiv) and [[2-(2,6-dioxopiperidin-3-yl)-1,3-dioxoisoindol-4-yl]oxy]acetic acid (32.70 mg, 0.098 mmol, 1.00 equiv) in DMF (1 mL) was added HATU (56.12 mg, 0.148 mmol, 1.50 equiv) and DIEA (31.80 mg, 0.246 mmol, 10 equiv). The mixture was stirred at 25° C. for 1 hour. The mixture was purified by prep-HPLC (conditions: SunFire Prep C18 OBD Column 19×150 mm 5 μm 10 nm; Mobile Phase A: Water (0.1% FA), Mobile Phase B: ACN; Flow rate: 25 mL/minute; Gradient: 8% B to 21% B in 10 minutes; 254/220 nm; Rt: 8.20 minutes) to afford 4-[2-(6-[[2,6-dimethoxy-4-(2-methyl-1-oxo-2,7-naphthyridin-4-yl)phenyl]methyl]-2,6-diazaspiro[3.3]heptan-2-yl)-2-oxoethoxy]-2-(2,6-dioxopiperidin-3-yl)isoindole-1,3-dione (6.2 mg, 8.74%) as a white solid. ¹H NMR (300 MHz, Methanol-d4) δ 9.51 (s, 1H), 8.68 (d, J=5.8 Hz, 1H), 8.55 (br s, 0.46H, FA), 7.80 (s, 1H), 7.69 (t, J=8.1 Hz, 1H), 7.62 (d, J=5.7 Hz, 1H), 7.44 (dd, J=11.8, 7.2 Hz, 1H), 7.29 (d, J=8.5 Hz, 1H), 6.87 (s, 2H), 5.19-5.10 (m, 1H), 4.69-4.51 (m, 6H), 4.39 (s, 2H), 4.34-4.26 (m, 2H), 4.22 (s, 2H), 3.97 (s, 6H), 3.69 (s, 3H), 2.95-2.68 (m, 3H), 2.20-2.09 (m, 1H). LCMS (ESI) m/z: [M+H]+=721.35.

### Example 43—Preparation of 4-(2-(4-(2,6-dimethoxy-4-(2-methyl-1-oxo-1,2-dihydro-2,7-naphthyridin-4-yl)benzyl)piperazin-1-yl)-2-oxoethoxy)-2-(2,6-dioxopiperidin-3-yl)isoindoline-1,3-dione formic acid (compound D37 formic acid)

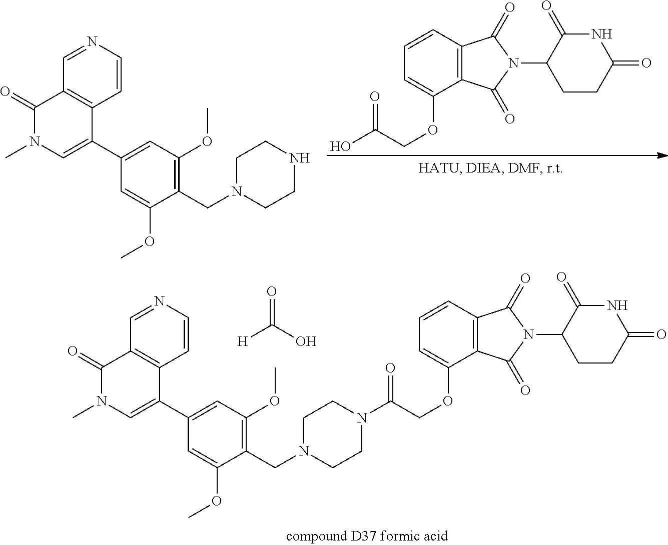

To a stirred solution of 4-[3,5-dimethoxy-4-(piperazin-1-ylmethyl)phenyl]-2-methyl-2,7-naphthyridin-1-one (50.0 mg, 0.127 mmol, 1.00 equiv) and [[2-(2,6-dioxopiperidin-3-yl)-1,3-dioxoisoindol-4-yl]oxy]acetic acid (42.1 mg, 0.127 mmol, 1.00 equiv) in DMF (1 mL) was added DIEA (163.8 mg, 1.268 mmol, 10.00 equiv) dropwise at room temperature. The resulting mixture was stirred for 10 minutes at room temperature. To the above mixture was added HATU (96.4 mg, 0.254 mmol, 2.00 equiv). The resulting mixture was stirred for additional 2 hours at room temperature. The residue was purified by reverse flash chromatography (conditions: SunFire C18 OBD Prep Column, 100 Å, 5 μm, 19 mm×250 mm; Mobile Phase A: Water (0.1% FA), Mobile Phase B: ACN; Flow rate: 25 mL/minute; Gradient: 9 B to 27 B in 10 minutes; 254 nm; R_{T}: 10.12 minutes) to afford 4-[2-(4-[[2,6-dimethoxy-4-(2-methyl-1-oxo-2,7-naphthyridin-4-yl)phenyl]methyl]piperazin-1-yl)-2-oxoethoxy]-2-(2,6-dioxopiperidin-3-yl)isoindole-1,3-dione formic acid (12.2 mg, 13.6%) as a white solid. ¹H NMR (400 MHz, Methanol-d4) δ 9.54 (s, 1H), 8.69 (d, J=5.8 Hz, 1H), 8.34 (br s, 0.28H, FA), 7.83-7.73 (m, 2H), 7.67-7.61 (m, 1H), 7.52 (d, J=7.1 Hz, 1H), 7.40 (d, J=8.5 Hz, 1H), 6.81 (s, 2H), 5.15-5.09 (m, 3H), 4.08 (s, 2H), 3.92 (s, 6H), 3.83-3.73 (m, 4H), 3.72 (s, 3H), 3.05-2.96 (m, 2H), 2.96-2.80 (m, 3H), 2.77-2.69 (m, 2H), 2.17-2.11 (m, 1H). LCMS (ESI) m/z: [M+H]+=709.35.

### Example 44—Preparation of 1-[[2,6-dimethoxy-4-(2-methyl-1-oxo-2,7-naphthyridin-4-yl)phenyl]methyl]-N-[2-[2-(2-[[2-(2,6-dioxopiperidin-3-yl)-1,3-dioxoisoindol-4-yl]amino]ethoxy)ethoxy]ethyl]azetidine-3-sulfonamide formic acid (compound D38 formic acid)

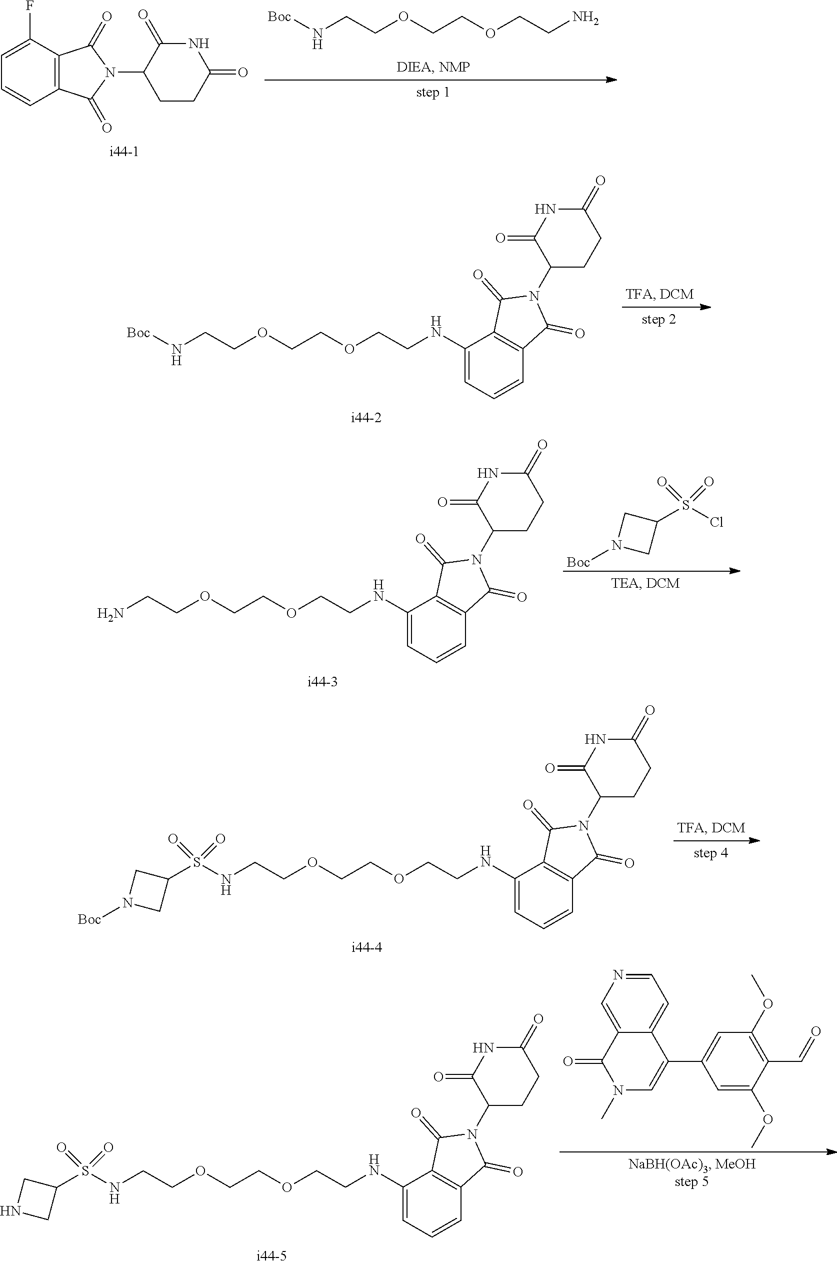

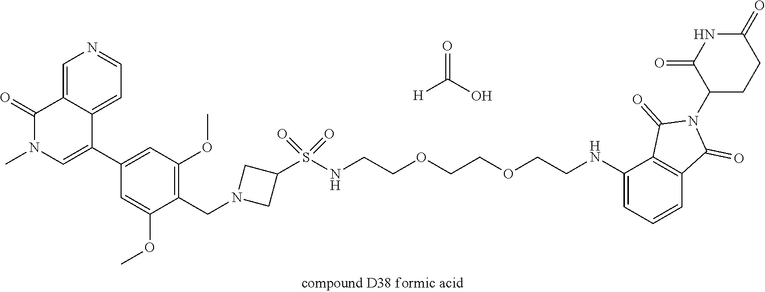 [SMILES: COc1cc(-c2cn(C)c(=O)c3cnccc23)cc(OC)c1CN1CC(S(=O)(=O)NCCOCCOCCNc2cccc3c2C(=O)N(C2CCC(=O)NC2=O)C3=O)C1 ; O=CO]

### Step 1: Preparation of tert-butyl N-[2-[2-(2-[[2-(2,6-dioxopiperidin-3-yl)-1,3-dioxo-2,3-dihydro-1H-isoindol-4-yl]amino]ethoxy)ethoxy]ethyl]carbamate (i44-2)

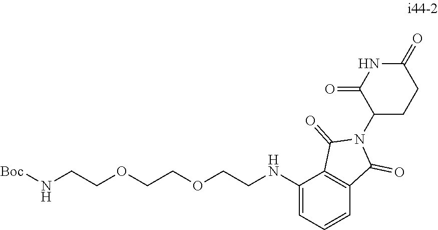 [SMILES: CC(C)(C)OC(=O)NCCOCCOCCNc1cccc2c1C(=O)N(C1CCC(=O)NC1=O)C2=O]

To a stirred solution of 2-(2,6-dioxopiperidin-3-yl)-4-fluoro-2,3-dihydro-1H-isoindole-1,3-dione (1.0 g, 3.620 mmol, 1.00 equiv) in NMP (15.00 mL) was added DIEA (940.47 mg, 7.277 mmol, 2.01 equiv) and tert-butyl N-[2-[2-(2-aminoethoxy)ethoxy]ethyl]carbamate (988.89 mg, 3.982 mmol, 1.10 equiv) in portions at room temperature. The resulting solution was stirred for 12 hours at 90° C. The resulting mixture was washed with water (3×100 mL). The resulting solution was extracted with ethyl acetate (3×200 mL). The organic layers combined and concentrated. This resulted in tert-butyl N-[2-[2-(2-[[2-(2,6-dioxopiperidin-3-yl)-1,3-dioxo-2,3-dihydro-1H-isoindol-4-yl]amino]ethoxy)ethoxy]ethyl]carbamate (1.2 g, 65.70%) as light yellow oil. LCMS (ESI) m/z: [M+H]+=505.

### Step 2: Preparation of 4-([2-[2-(2-aminoethoxy)ethoxy]ethyl]amino)-2-(2,6-dioxopiperidin-3-yl)-2,3-dihydro-1H-isoindole-1,3-dione (i44-3)

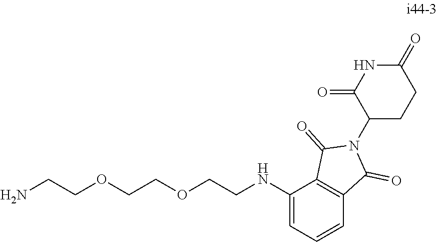 [SMILES: NCCOCCOCCNc1cccc2c1C(=O)N(C1CCC(=O)NC1=O)C2=O]

To a stirred solution of tert-butyl N-[2-[2-(2-[[2-(2,6-dioxopiperidin-3-yl)-1,3-dioxo-2,3-dihydro-1H-isoindol-4yl]amino]ethoxy)ethoxy]ethyl]carbamate (1.2 g, 2.378 mmol, 1.00 equiv) in DCM (40 mL) was added TFA (10 mL) in portions at room temperature. The resulting solution was stirred for 4 hours at room temperature. The resulting mixture was concentrated. This resulted in 4-([2-[2-(2-aminoethoxy)ethoxy]ethyl]amino)-2-(2,6-dioxopiperidin-3-yl)-2,3-dihydro-1H-isoindole-1,3-dione (0.8 g, 83.17%) as light yellow oil. LCMS (ESI) m/z: [M+H]+=405.

### Step 3: Preparation of tert-butyl 3-([2-[2-(2-[[2-(2,6-dioxopiperidin-3-yl)-1,3-dioxoisoindol-4-yl]amino]eth oxy)ethoxy]ethyl]sulfamoyl)azetidine-1-carboxylate (i44-4)

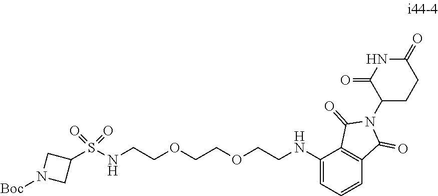 [SMILES: CC(C)(C)OC(=O)N1CC(S(=O)(=O)NCCOCCOCCNc2cccc3c2C(=O)N(C2CCC(=O)NC2=O)C3=O)C1]

To a stirred solution of 4-([2-[2-(2-aminoethoxy)ethoxy]ethyl]amino)-2-(2,6-dioxopiperidin-3-yl)isoindole-1,3-dione (238.00 mg, 0.588 mmol, 1.00 equiv) in DCM was added TEA (120.00 mg, 1.186 mmol, 2.02 equiv) in portions at room temperature. To the above mixture was added tert-butyl 3-(chlorosulfonyl) azetidine-1-carboxylate (150.00 mg, 0.587 mmol, 1.00 equiv) in portions. The resulting mixture was stirred for additional 2 hours at room temperature. The resulting mixture was concentrated under reduced pressure. The residue was purified by silica gel column chromatography, eluted with CH₂Cl₂/EtOAc (1:1) to afford tert-butyl 3-([2-[2-(2-[[2-(2,6-dioxopiperidin-3-yl)-1,3-dioxoisoindol-4-yl]amino]ethoxy)ethoxy]ethyl]sulfamoyl)azetidine-1-carboxylate (130 mg, 35.42%) as a light yellow oil. LCMS (ESI) m/z: [M+H]+=624.

### Step 4: Preparation of N-(2-(2-(2-((2-(2, 6-dioxopiperidin-3-yl)-1,3-dioxoisoindolin-4-yl)amino)ethoxy)ethox-y)ethy)azetidine-3-sulfonamide (i44-5)

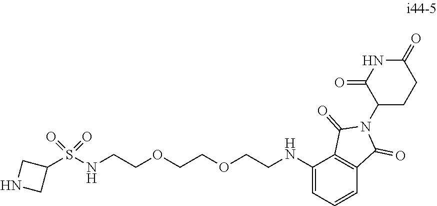 [SMILES: O=C1CCC(N2C(=O)c3cccc(NCCOCCOCCNS(=O)(=O)C4CNC4)c3C2=O)C(=O)N1]

To a stirred solution/mixture of tert-butyl 3-([2-[2-(2-[[2-(2,6-dioxopiperidin-3-yl)-1,3-dioxoisoindol-4-yl]amino]ethoxy)ethoxy]ethyl]sulfamoyl)azetidine-1-carboxylate (120.00 mg, 0.192 mmol, 1.00 equiv) in DCM (4 mL) was added TFA (1 mL) in portions at room temperature. The resulting mixture was stirred for 1 hour at room temperature. The resulting mixture was concentrated under reduced pressure. The crude product 130 mg was used in the next step directly without further purification. LCMS (ESI) m/z: [M+H]+=524.

### Step 5: Preparation of 1-[[2,6-dimethoxy-4-(2-methyl-1-oxo-2,7-naphthyridin-4-yl)phenyl]methyl]-N-[2-[2-(2-[[2-(2,6-dioxopiperidin-3-yl)-1,3-dioxoisoindol-4-yl]amino]ethoxy)ethoxy]ethyl]azetidine-3-sulfonamide formic acid (compound D38 formic acid)

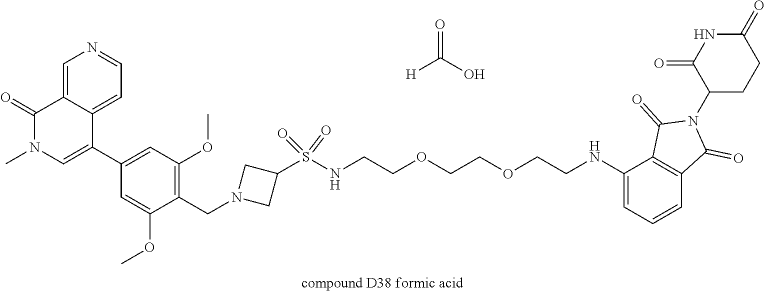 [SMILES: COc1cc(-c2cn(C)c(=O)c3cnccc23)cc(OC)c1CN1CC(S(=O)(=O)NCCOCCOCCNc2cccc3c2C(=O)N(C2CCC(=O)NC2=O)C3=O)C1 ; O=CO]

To a stirred solution of N-[2-[2-(2-[[2-(2,6-dioxopiperidin-3-yl)-1,3-dioxoisoindol-4-yl]amino]ethoxy) ethoxy]ethyl]azetidine-3-sulfonamide (60.00 mg, 0.115 mmol, 1.00 equiv) and 2,6-dimethoxy-4-(2-methyl-1-oxo-2,7-naphthyridin-4-yl)benzaldehyde (74.34 mg, 0.229 mmol, 2.00 equiv) in MeOH was added NaBH(OAc)₃ (97.15 mg, 0.458 mmol, 4.00 equiv) in portions at room temperature. The resulting mixture was stirred for 12 hours at room temperature. The crude product was purified by Prep-HPLC (conditions: SunFire Prep C18 OBD Column, 19*150 mm 5 μm 10 nm; mobile phase, Water (0.1% FA) and ACN (10% Phase B up to 27% in 8 minutes); Detector, UV). This resulted in 1-[[2,6-dimethoxy-4-(2-methyl-1-oxo-2,7-naphthyridin-4-yl)phenyl]methyl]-N-[2-[2-(2[[2-(2,6-dioxopiperidin-3-yl)-1,3-dioxoisoindol-4-yl]amino]ethoxy)ethoxy]ethyl]azetidine-3-sulfonamide formic acid (8.1 mg, 8.05%) as a yellow solid. ¹H NMR (300 MHz, Methanol-d4) δ 9.49 (d, J=0.9 Hz, 1H), 8.66 (d, J=5.8 Hz, 1H), 8.45 (br s, 1H, FA), 7.74 (s, 1H), 7.62 (dd, J=5.8, 0.9 Hz, 1H), 7.50 (dd, J=8.5, 7.1 Hz, 1H), 7.02 (dd, J=7.8, 5.3 Hz, 2H), 6.79 (s, 2H), 5.07 (dd, J=12.4, 5.4 Hz, 1H), 4.61 (s, 1H), 4.36-4.23 (m, 1H), 4.20 (s, 2H), 4.13-3.99 (m, 4H), 3.92 (s, 6H), 3.73-3.64 (m, 9H), 3.55 (t, J=5.1 Hz, 2H), 3.50-3.41 (m, 2H), 3.28 (t, J=5.1 Hz, 2H), 2.96-2.61 (m, 3H), 2.18-2.04 (m, 1H). LCMS (ESI) m/z: [M+H]+=832.45.

### Example 45—Preparation of 4-[4-(9-[[2,6-dimethoxy-4-(2-methyl-1-oxo-2,7-naphthyridin-4-yl)phenyl]methyl]-1-oxa-4,9-diazaspiro[5.5]undecan-4-yl)-4-oxobutoxy]-2-(2,6-dioxopiperidin-3-yl)isoindole-1,3-dione. (compound D39)

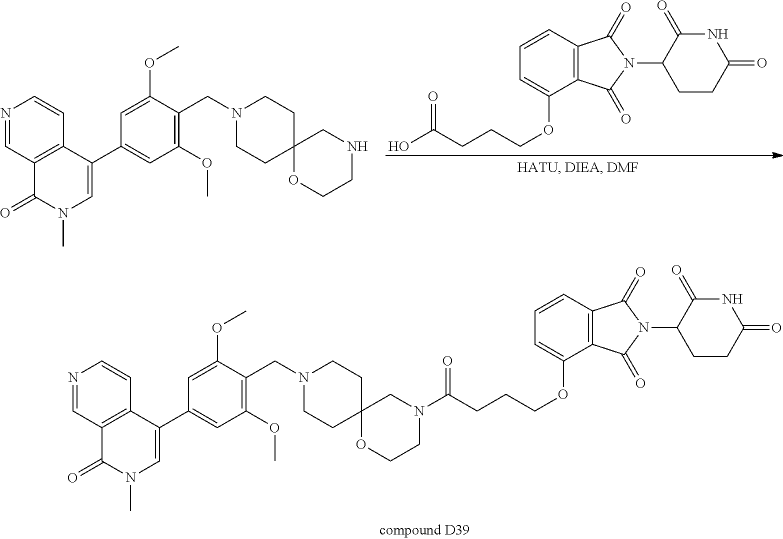 [SMILES: COc1cc(-c2cn(C)c(=O)c3cnccc23)cc(OC)c1CN1CCC2(CC1)CNCCO2 ; O=C(O)CCCOc1cccc2c1C(=O)N(C1CCC(=O)NC1=O)C2=O ; COc1cc(-c2cn(C)c(=O)c3cnccc23)cc(OC)c1CN1CCC2(CC1)CN(C(=O)CCCOc1cccc3c1C(=O)N(C1CCC(=O)NC1=O)C3=O)CCO2]

To a stirred solution of 4-(3,5-dimethoxy-4-[1-oxa-4,9-diazaspiro[5.5]undecan-9-ylmethyl]phenyl)-2-methyl-2,7naphthyridin-1-one (20.00 mg, 0.043 mmol, 1.00 equiv) and 4-[[2-(2,6-dioxopiperidin-3-yl)-1,3-dioxoisoindol-4-yl]oxy]butanoic acid (15.00 mg, 0.042 mmol, 0.97 equiv) in DMF was added HATU (25.00 mg, 0.066 mmol, 1.53 equiv) and DIEA (60.00 mg, 0.464 mmol, 10.78 equiv) in portions at room temperature. The resulting mixture was stirred for 2 hours at room temperature. The crude product was purified by Prep-HPLC (conditions: Gemini-NX C18 AXAI Packed, 21.2*150 mm 5 μm; mobile phase, Water (0.1% FA) and ACN (14% Phase B up to 19% in 10 minutes); Detector, UV). This resulted in 4-[4-(9-[[2,6-dimethoxy-4-(2-methyl-1-oxo-2,7-naphthyridin-4-yl)phenyl]methyl]-1-oxa-4,9diaza spiro[5.5]undecan-4-yl)-4-oxobutoxy]-2-(2,6-dioxopiperidin-3-yl)isoindole-1,3-dione (5.1 mg, 14.68%) as a white solid. ¹H NMR (300 MHz, Methanol-d4) δ 9.55 (s, 1H), 8.70 (d, J=5.6 Hz, 1H), 7.85-7.75 (m, 2H), 7.63 (d, J=5.8 Hz, 1H), 7.57-7.43 (m, 2H), 6.87 (d, J=5.2 Hz, 2H), 5.12 (d, J=11.8 Hz, 1H), 4.41 (s, 2H), 4.37-4.27 (m, 2H), 3.96 (d, J=8.2 Hz, 6H), 3.84-3.60 (m, 9H), 3.58-3.45 (m, 3H), 2.92-2.69 (m, 5H), 2.26-2.04 (m, 6H), 1.85-1.60 (m, 2H). LCMS (ESI) m/z: [M+H]⁺=807.40.

### Example 46—Preparation of 4-[[5-(9-[[2,6-dimethoxy-4-(2-methyl-1-oxo-2,7-naphthyridin-4-yl)phenyl]methyl]-1-oxa-4,9-diazaspiro[5.5]undecan-4-yl)-5-oxopentyl]oxy]-2-(2,6-dioxopiperidin-3-yl)isoindole-1,3-dione formic acid (compound D40 formic acid)

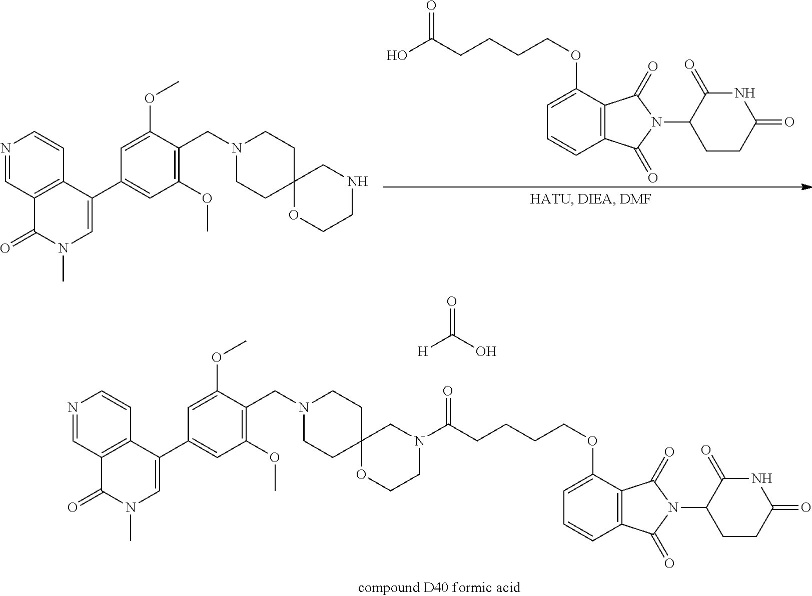 [SMILES: COc1cc(-c2cn(C)c(=O)c3cnccc23)cc(OC)c1CN1CCC2(CC1)CNCCO2 ; O=C(O)CCCCOc1cccc2c1C(=O)N(C1CCC(=O)NC1=O)C2=O ; COc1cc(-c2cn(C)c(=O)c3cnccc23)cc(OC)c1CN1CCC2(CC1)CN(C(=O)CCCCOc1cccc3c1C(=O)N(C1CCC(=O)NC1=O)C3=O)CCO2 ; O=CO]

To a stirred solution of 4-(3,5-dimethoxy-4-[1-oxa-4,9-diazaspiro[5.5]undecan-9-ylmethyl]phenyl)-2-methyl-2,7-naphthyridin-1-one (30.00 mg, 0.065 mmol, 1.00 equiv) and 5-[[2-(2,6-dioxopiperidin-3-yl)-1,3-dioxoisoindol-4-yl]oxy]pentanoic acid (24.17 mg, 0.065 mmol, 1 equiv) in DMF (1.00 mL) was added DIEA (83.46 mg, 0.646 mmol, 10.00 equiv) and HATU (36.83 mg, 0.097 mmol, 1.50 equiv). The resulting solution was stirred at room temperature for 1 hour. Without any additional work-up, the mixture was purified by prep-HPLC (conditions: SunFire C18 OBD Prep Column, 100 Å, 5 μm, 19 mm×250 mm; Mobile Phase A: Water (0.1% FA), Mobile Phase B: ACN; Flow rate: 25 mL/minute; Gradient: 9% B to 25% B in 10 minutes; 254 nm; Rₜ: 10.95 minutes) to give (4-[[5-(9-[[2,6-dimethoxy-4-(2-methyl-1-oxo-2,7-naphthyridin-4-yl)phenyl]methyl]-1-oxa-4,9-diazaspiro[5.5]undecan-4-yl)-5-oxopentyl]oxy]-2-(2,6-dioxopiperidin-3-yl)isoindole-1,3-dione formic acid (8.6 mg, 15.25%) as a white solid. ¹H NMR (300 MHz, Methanol-d4) δ 9.54 (s, 1H), 8.69 (d, J=5.8 Hz, 1H), 8.53 (br s, 1H, FA), 7.85-7.74 (m, 2H), 7.62 (dd, J=5.9, 0.9 Hz, 1H), 7.46 (dd, J=7.8, 2.3 Hz, 2H), 6.86 (d, J=5.7 Hz, 2H), 5.12 (dd, J=12.3, 5.4 Hz, 1H), 4.39 (s, 2H), 4.35-4.25 (m, 3H), 3.96 (s, 6H), 3.83-3.74 (m, 2H), 3.72 (s, 3H), 3.67-3.61 (m, 2H), 3.55-3.50 (m, 3H), 3.00-2.51 (m, 6H), 2.20-1.71 (m, 10H). LCMS (ESI) m/z: [M+H]+=821.45.

### Example 47—Preparation of 4-[2-(9-[[2,6-dimethoxy-4-(2-methyl-1-oxo-2,7-naphthyridin-4-yl)phenyl]methyl]-1-oxa-4,9-diazaspiro[5.5]undecan-4-yl)-2-oxoethoxy]-2-(2,6-dioxopiperidin-3-yl)isoindole-1,3-dione formic acid (compound D41 formic acid)

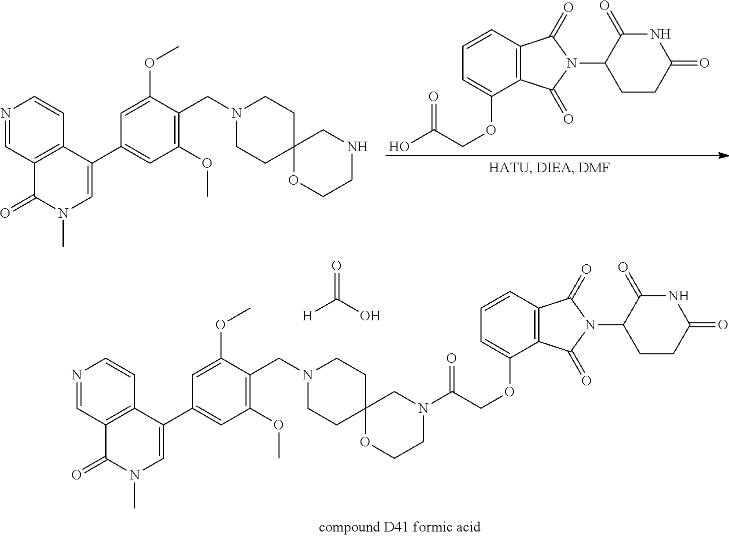 [SMILES: COc1cc(-c2cn(C)c(=O)c3cnccc23)cc(OC)c1CN1CCC2(CC1)CNCCO2 ; O=C(O)COc1cccc2c1C(=O)N(C1CCC(=O)NC1=O)C2=O ; COc1cc(-c2cn(C)c(=O)c3cnccc23)cc(OC)c1CN1CCC2(CC1)CN(C(=O)COc1cccc3c1C(=O)N(C1CCC(=O)NC1=O)C3=O)CCO2 ; O=CO]

To a solution of 4-(3,5-dimethoxy-4-[1-oxa-4,9-diazaspiro[5.5]undecan-9-ylmethyl]phenyl)-2-methyl-2,7-naphthyridin-1-one (30.00 mg, 0.065 mmol, 1.00 equiv) and [[2-(2,6-dioxopiperidin-3-yl)-1,3-dioxoisoindol-4-yl]oxy]acetic acid (21.46 mg, 0.065 mmol, 1.00 equiv) in DMF (1.00 mL) was added DIEA (83.46 mg, 0.646 mmol, 10.00 equiv) and HATU (36.83 mg, 0.097 mmol, 1.50 equiv). The resulting solution was stirred at room temperature for 1 hour. Without any additional work-up, the mixture was purified by prep-HPLC (conditions: Phenomenex Gemini C6-Phenyl, 21.2*250 mm, 5 μm; Mobile Phase A: Water (0.05% FA), Mobile Phase B:ACN; Flow rate:25 mL/minute; Gradient:7 B to 26 B in 15 minutes; 254 nm; R_{T}: 14.62 minutes) to give 4-[2-(9-[[2,6-dimethoxy-4-(2-methyl-1-oxo-2,7-naphthyridin-4-yl)phenyl]methyl]-1-oxa-4,9-diazaspiro[5.5]undecan-4-yl)-2-oxoethoxy]-2-(2,6-dioxopiperidin-3-yl)isoindole-1,3-dione formic acid (3.7 mg, 6.80%) as a white solid. ¹H NMR (300 MHz, Methanol-d4) δ 9.54 (d, J=0.8 Hz, 1H), 8.70 (d, J=5.8 Hz, 1H), 8.56 (br s, 1H, FA), 7.86-7.75 (m, 2H), 7.63 (dd, J=5.8, 0.9 Hz, 1H), 7.54 (d, J=7.2 Hz, 1H), 7.44 (d, J=8.5 Hz, 1H), 6.86 (s, 2H), 5.17-5.07 (m, 3H), 4.30 (s, 2H), 3.95 (s, 6H), 3.87-3.75 (m, 2H), 3.72 (s, 3H), 3.68-3.62 (m, 2H), 3.54 (s, 2H), 3.23-3.17 (m, 4H), 2.91-2.65 (m, 3H), 2.22-2.02 (m, 3H), 1.80 (s, 2H). LCMS (ESI) m/z: [M+H]+=779.40.

### Example 48—Preparation of 5-(4-(2-(1-(2,6-dimethoxy-4-(2-methyl-1-oxo-1,2-dihydro-2,7-naphthyridin-4-yl)benzyl) piperidin-4-yl)ethyl)piperazin-1-yl)-2-(2,6-dioxopiperidin-3-yl)isoindoline-1,3-dione formic acid (compound D42 formic acid)

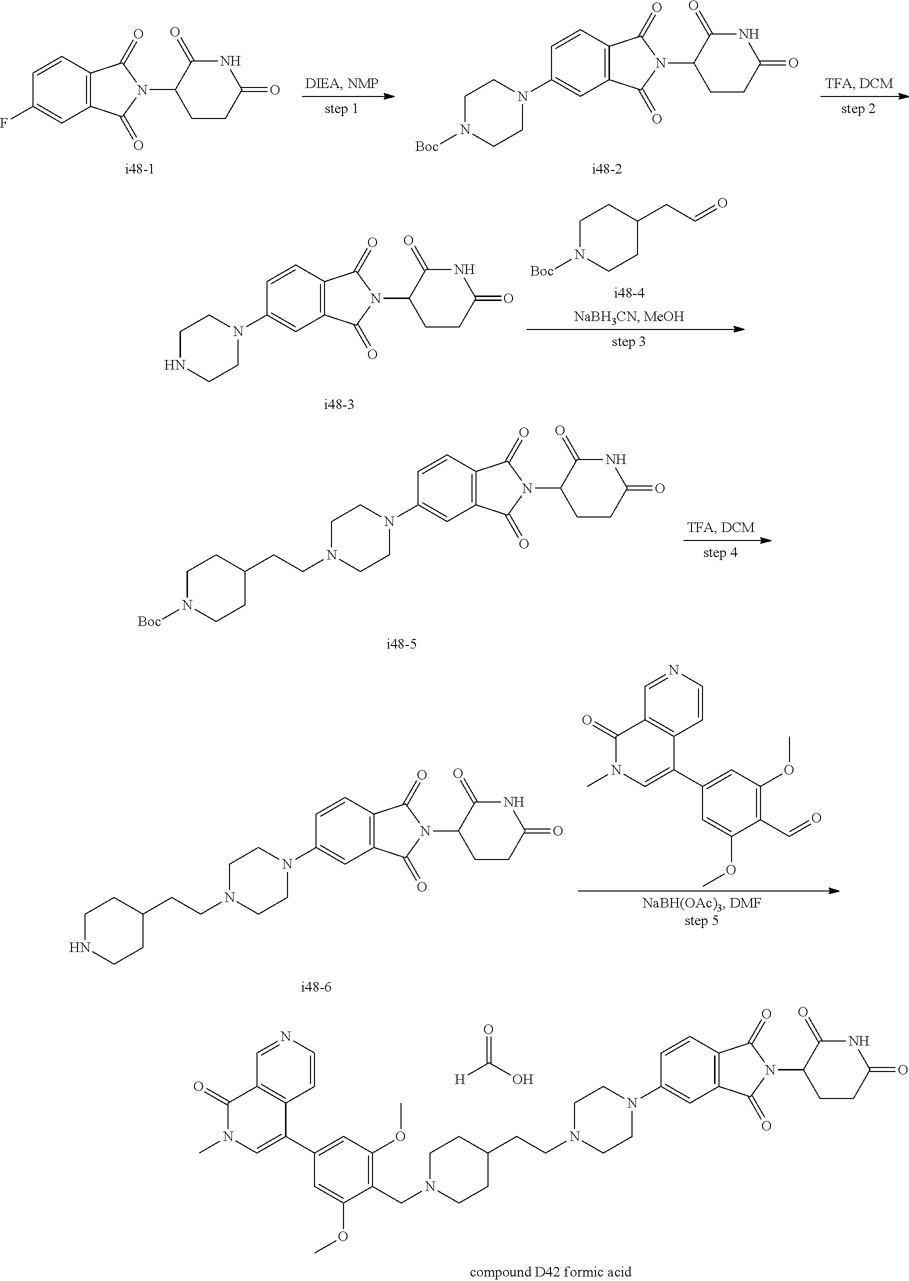

### Step 1: Preparation of tert-butyl 4-(2-(2,6-dioxopiperidin-3-yl)-1,3-dioxoisoindolin-5-yl)piperazine-1-carboxylate (i42-2)

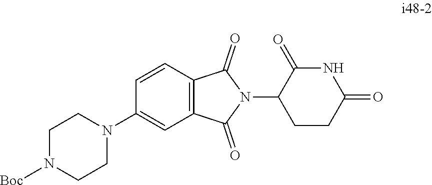 [SMILES: CC(C)(C)OC(=O)N1CCN(c2ccc3c(c2)C(=O)N(C2CCC(=O)NC2=O)C3=O)CC1]

To a solution of 2-(2,6-dioxopiperidin-3-yl)-5-fluoroisoindole-1,3-dione (1.38 g, 4.996 mmol, 1.00 equiv) and tert-butyl piperazine-1-carboxylate (930.52 mg, 4.996 mmol, 1.00 equiv) in NMP (20 mL) was added DIPEA (1937.08 mg, 14.988 mmol, 3 equiv). The mixture was stirred at 90° C. for 2 hours (under nitrogen atmosphere). The reaction was monitored by LC-MS. The resulting mixture was diluted with water (70 mL) and then extracted with EA (3×25 mL). The combined organic layers were washed with water (2×25 mL) and dried over anhydrous Na₂SO₄. After filtration, the filtrate was concentrated under reduced pressure. The resulting mixture was concentrated under vacuum. The residue was purified by reverse flash chromatography (conditions: column, C18 silica gel; mobile phase, 0.5% FA in water, 10% to 90% gradient in 25 minutes; detector, UV 220 nm). The fractions were concentrated under reduced pressure afford tert-butyl 4-(2-(2,6-dioxopiperidin-3-yl)-1,3-dioxoisoindolin-5-yl)piperazine-1-carboxylate (700 mg, 31.67%) as a yellow solid. LCMS (ESI) m/z: [M+H]+=443.

### Step 2: Preparation of 2-(2,6-dioxopiperidin-3-yl)-5-(piperazin-1-yl)isoindoline-1,3-dione (i42-3)

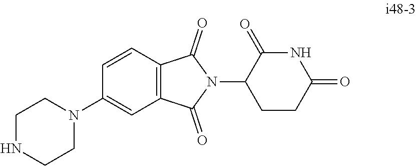 [SMILES: O=C1CCC(N2C(=O)c3ccc(N4CCNCC4)cc3C2=O)C(=O)N1]

A solution of tert-butyl 4-[2-(2,6-dioxopiperidin-3-yl)-1,3-dioxoisoindol-5-yl]piperazine-1-carboxylate (500.00 mg, 1.130 mmol, 1.00 equiv) and TFA (1.50 mL, 20.195 mmol, 17.87 equiv) in DCM (5.00 mL) was stirred at 25° C. for 1 hour. The resulting mixture were evaporated to dryness to afford 2-(2,6-dioxopiperidin-3-yl)-5-(piperazin-1-yl)isoindole-1,3-dione (350 mg, 90.47%) as a brown solid. LCMS (ESI) m/z: [M+H]+=343

### Step 3: Preparation of tert-butyl 4-(2-(4-(2-(2,6-dioxopiperidin-3-yl)-1,3-dioxoisoindolin-5-yl)piperazin-1-yl)ethyl)piperidine-1-carboxylate (i42-5)

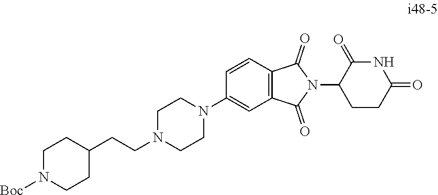 [SMILES: CC(C)(C)OC(=O)N1CCC(CCN2CCN(c3ccc4c(c3)C(=O)N(C3CCC(=O)NC3=O)C4=O)CC2)CC1]

To a solution of 2-(2,6-dioxopiperidin-3-yl)-5-(piperazin-1-yl)isoindole-1,3-dione (200.00 mg, 0.584 mmol, 1.00 equiv) and tert-butyl 4-(2-oxoethyl)piperidine-1-carboxylate (132.79 mg, 0.584 mmol, 1 equiv) in DMF (3.00 mL) was added NaBH(OAc)₃ (247.63 mg, 1.168 mmol, 2 equiv). The resulting solution was stirred at 25° C. for 1 hour. The residue was purified by reverse flash chromatography (conditions: column, C18 silica gel; mobile phase, ACN in water, 10% to 50% gradient in 10 minutes; detector, UV 254 nm) to give tert-butyl 4-(2-[4-[2-(2,6-dioxopiperidin-3-yl)-1,3-dioxoisoindol-5-yl]piperazin-1-yl]ethyl)piperidine-1-carboxylate (197.5 mg, 61.06%) as a yellow solid. LCMS (ESI) m/z: [M+H]+=554.

### Step 4: Preparation of 2-(2,6-dioxopiperidin-3-yl)-5-(4-(2-(piperidin-4-yl)ethyl)piperazin-1-yl)isoindoline-1,3-dione (i42-6)

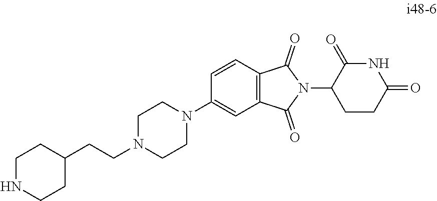 [SMILES: O=C1CCC(N2C(=O)c3ccc(N4CCN(CCC5CCNCC5)CC4)cc3C2=O)C(=O)N1]

To a solution of tert-butyl 4-(2-[4-[2-(2,6-dioxopiperidin-3-yl)-1,3-dioxoisoindol-5-yl]piperazin-1-yl]ethyl)piperidine-1-carboxylate (197.00 mg, 0.356 mmol, 1.00 equiv) and TFA (0.50 mL, 6.732 mmol, 18.92 equiv) in DCM (2.00 mL) was stirred at 25° C. for 1 hour. The mixture was concentrated to give crude product 2-(2,6-dioxopiperidin-3-yl)-5-[4-[2-(piperidin-4-yl)ethyl]piperazin-1-yl]isoindole-1,3-dione (320 mg) as a yellow oil, that was used directly without further purification. LCMS (ESI) m/z: [M+H]+=454.

### Step 5: Preparation of 5-(4-(2-(1-(2,6-dimethoxy-4-(2-methyl-1-oxo-1,2-dihydro-2,7-naphthyridin-4-yl)benzyl)piperidin-4-yl)ethyl)piperazin-1-yl)-2-(2,6-dioxopiperidin-3-yl)isoindoline-1,3-dione formic acid (compound D42 formic acid)

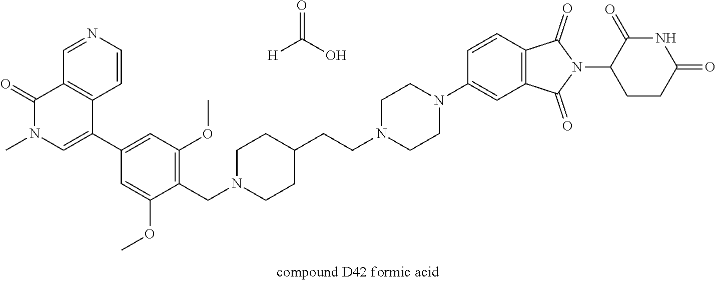 [SMILES: COc1cc(-c2cn(C)c(=O)c3cnccc23)cc(OC)c1CN1CCC(CCN2CCN(c3ccc4c(c3)C(=O)N(C3CCC(=O)NC3=O)C4=O)CC2)CC1 ; O=CO]

To a solution of 2-(2,6-dioxopiperidin-3-yl)-5-[4-[2-(piperidin-4-yl)ethyl]piperazin-1-yl]isoindole-1,3-dione(100.68 mg, 0.222 mmol, 1.20 equiv) and 2,6-dimethoxy-4-(2-methyl-1-oxo-2,7-naphthyridin-4-yl)benzaldehyde (60.00 mg, 0.185 mmol, 1.00 equiv) in DMF (1.5 mL) was added NaBH(OAc)₃ (78.42 mg, 0.370 mmol, 2 equiv). The mixture was stirred at 25° C. for 1 hour. The mixture was purified by prep-HPLC (conditions: SunFire C18 OBD Prep Column, 100 Å, 5 μm, 19 mm×250 mm; Mobile Phase A: Water (0.1% FA), Mobile Phase B:ACN; Flow rate:25 mL/minute; Gradient:10 B to 12 B in 10 minutes; 254 nm; R_{T}: 8.7 minutes) to afford 5-[4-[2-(1-[[2,6-dimethoxy-4-(2-methyl-1-oxo-2,7-naphthyridin-4-yl)phenyl]methyl]piperidin-4-yl)ethyl]piperazin-1-yl]-2-(2,6-dioxopiperidin-3-yl)isoindole-1,3-dione (24 mg, 17.03%) as a yellow solid. ¹H NMR (300 MHz, Methanol-d4) δ 9.55 (d, J=0.9 Hz, 1H), 8.69 (d, J=5.8 Hz, 1H), 8.15 (br s, 0.2H, FA), 7.80-7.71 (m, 2H), 7.63 (d, J=5.8 Hz, 1H), 7.43 (s, 1H), 7.31 (d, J=9.2 Hz, 1H), 6.89 (s, 2H), 5.10 (dd, J=12.3, 5.4 Hz, 1H), 4.41 (s, 2H), 3.98 (s, 6H), 3.72 (s, 3H), 3.67-3.55 (m, 6H), 3.17 (d, J=12.9 Hz, 2H), 3.05-2.92 (m, 4H), 2.90-2.70 (m, 5H), 2.17-2.00 (m, 3H), 1.81-1.51 (m, 5H). LCMS (ESI) m/z: [M+H]+=762.45.

### Example 49—Preparation of 5-[2-(6-[[2,6-Dimethoxy-4-(2-methyl-1-oxo-2,7-naphthyridin-4-yl)phenyl]methyl]-2,6-diazaspiro[3.3]heptan-2-yl)ethoxy]-2-(2,6-dioxopiperidin-3-yl)isoindole-1,3-dione formic acid (compound D43 formic acid)

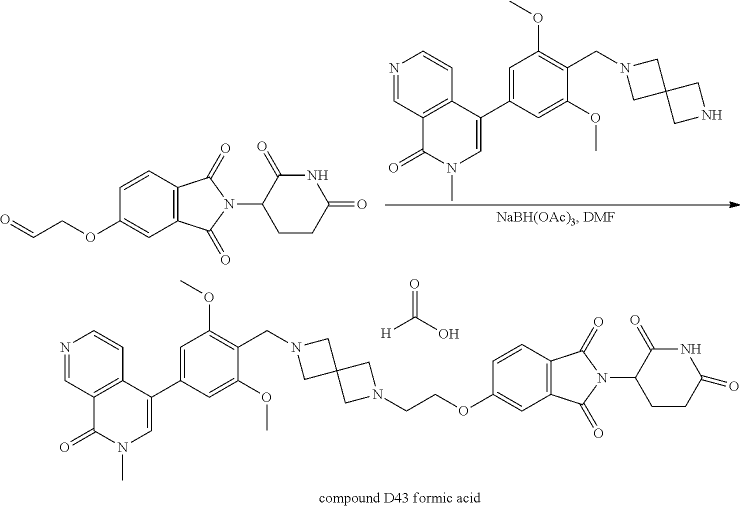

To a solution of 2-[[2-(2,6-dioxopiperidin-3-yl)-1,3-dioxoisoindol-5-yl]oxy]acetaldehyde (60.00 mg, 0.190 mmol, 1.00 equiv) and 4-(4-[2,6-diazaspiro[3.3]heptan-2-ylmethyl]-3,5-dimethoxyphenyl)-2-methyl-2,7-naphthyridin-1-one (77.12 mg, 0.190 mmol, 1 equiv) in DMF (1.00 mL)was added NaBH(OAc)₃ (80.42 mg, 0.379 mmol, 2 equiv). The resulting solution was stirred at room temperature for 1 hour. The crude product (60 mg) was purified by Prep-HPLC (conditions: SunFire Prep C18 OBD Column 19×150 mm 5 μm 10 nm; Mobile Phase A: Water (0.1% FA), Mobile Phase B: ACN; Flow rate: 25 mL/minute; Gradient: 7% B to 10% B in 12 minutes; 254/220 nm; Rt: 9.65 minutes) to afford 5-[2-(6-[[2,6-dimethoxy-4-(2-methyl-1-oxo-2,7-naphthyridin-4-yl)phenyl]methyl]-2,6-diazaspiro[3.3]heptan-2-yl)ethoxy]-2-(2,6-dioxopiperidin-3-yl)isoindole-1,3-dione formic acid (14.3 mg, 9.82%) as a light yellow solid. ¹H NMR (300 MHz, Methanol-d4) δ 9.54 (s, 1H), 8.68 (d, J=5.8 Hz, 1H), 8.14 (br s, 0.2H, FA), 7.76 (s, 1H), 7.70 (d, J=8.2 Hz, 1H), 7.62-7.54 (m, 1H), 7.19 (d, J=2.2 Hz, 1H), 7.13 (dd, J=8.2, 2.2 Hz, 1H), 6.86 (s, 2H), 5.16 (dd, J=12.8, 5.4 Hz, 1H), 4.47 (s, 2H), 4.34 (s, 4H), 3.98 (s, 6H), 3.95-3.87 (m, 2H), 3.80 (s, 4H), 3.71 (s, 3H), 3.00-2.85 (m, 4H), 2.81-2.63 (m, 1H), 2.20-2.05 (m, 1H). LCMS (ESI) m/z: [M+H]⁺=707.5.

### Example 50—Preparation of 5-((5-(4-(2,6-dimethoxy-4-(2-methyl-1-oxo-1,2-dihydro-2,7-naphthyridin-4-yl)benzyl)piperazin-1-yl)pentyl)oxy)-2-(2,6-dioxopiperidin-3-yl)isoindoline-1,3-dione formic acid (compound D44 formic acid)

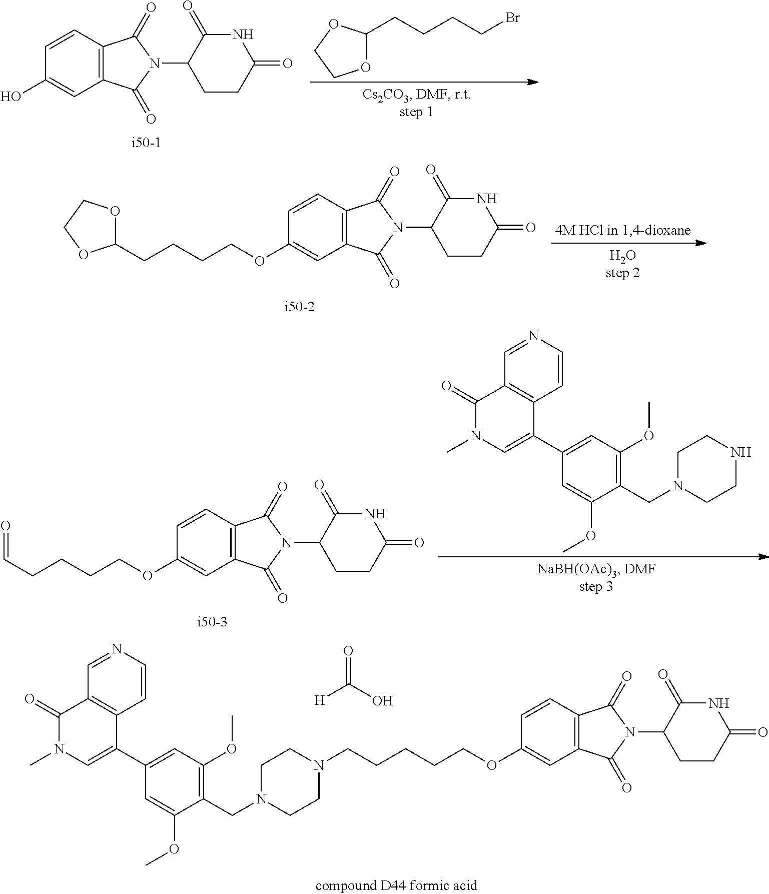

### Step 1: 5-(4-(1,3-dioxolan-2-yl)butoxy)-2-(2,6-dioxopiperidin-3-yl)isoindoline-1,3-dione (i50-2)

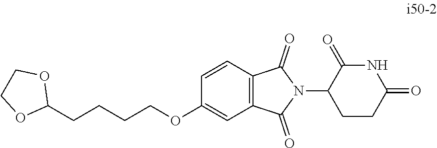 [SMILES: O=C1CCC(N2C(=O)c3ccc(OCCCCC4OCCO4)cc3C2=O)C(=O)N1]

To a stirred solution of 2-(2,6-dioxopiperidin-3-yl)-5-hydroxy-2,3-dihydro-1H-isoindole-1,3-dione (400.0 mg, 1.459 mmol, 1.00 equiv) and 2-(4-bromobutyl)-1,3-dioxolane (305.0 mg, 1.459 mmol, 1.00 equiv) in DMF was added cesium carbonate (475.3 mg, 1.459 mmol, 1.00 equiv) at room temperature. The resulting mixture was filtered, and the filter cake was washed with DCM (3×5 mL). The filtrate was concentrated under reduced pressure. The residue was purified by Prep-TLC (PE/EtOAc 1:1) to afford 5-[4-(1,3-dioxolan-2-yl)butoxy]-2-(2,6-dioxopiperidin-3-yl)-2,3-dihydro-1H-isoindole-1,3-dione (40 mg, 6.5%) as an off-white oil. LCMS (ESI) m/z: [M+H]+=403.

### Step 2: Preparation of 5-((2-(2,6-dioxopiperidin-3-yl)-1,3-dioxoisoindolin-5-yl)oxy)pentanal (i50-3)

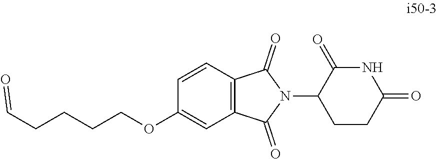 [SMILES: O=CCCCCOc1ccc2c(c1)C(=O)N(C1CCC(=O)NC1=O)C2=O]

To a stirred mixture of 5-[4-(1,3-dioxolan-2-yl)butoxy]-2-(2,6-dioxopiperidin-3-yl)isoindole-1,3-dione (40.0 mg, 0.099 mmol, 1.00 equiv) in water (1.50 mL) was added HCl in 1,4-dioxane (4 M, 3.00 mL) at room temperature. The resulting mixture was stirred for 2 hours at room temperature. The resulting mixture was extracted with EtOAc (2×10 mL). The combined organic layers were washed with brine (8 mL), and dried over anhydrous Na₂SO₄. After filtration, the filtrate was concentrated under reduced pressure. The crude product was used in the next step directly without further purification. LCMS (ESI) m/z: [M+H]⁺=359.

### Step 3: Preparation of N-(6-[4-[(dimethylamino)methyl]-3,5-dimethoxyphenyl]-3-methyl-[1,2,4]triazolo[4,3-a]pyridin-8-yl)acetamide formic acid (compound D44 formic acid)

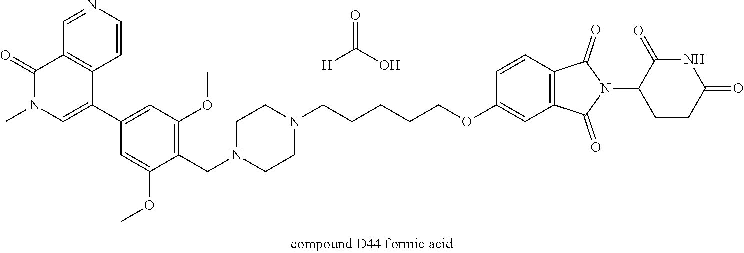

To a stirred solution/mixture of 5-[[2-(2,6-dioxopiperidin-3-yl)-1,3-dioxoisoindol-5-yl]oxy]pentanal (20 mg, 0.056 mmol, 1.00 equiv) in DMF (1 mL) was added 4-[3,5-dimethoxy-4-(piperazin-1-ylmethyl)phenyl]-2-methyl-2,7-naphthyridin-1-one (22.0 mg, 0.056 mmol, 1 equiv) at room temperature. The resulting mixture was stirred for 30 minutes at room temperature. To the above mixture was added NaBH(OAc)₃ (23.7 mg, 0.112 mmol, 2.00 equiv) at room temperature. The resulting mixture was stirred for additional 2 hours at room temperature. The crude product was purified by Prep-HPLC (conditions: SunFire Prep C18 OBD Column, 19×150 mm 5 μm 10 nm; Mobile Phase A: Water (0.1% FA), Mobile Phase B: ACN; Flow rate: 25 mL/minute; Gradient: 10 B to 25 B in 8 minutes; 254/220 nm; R_{T}: 6.53 minutes) to afford 5-[[5-(4-[[2,6-dimethoxy-4-(2-methyl-1-oxo-2,7-naphthyridin-4-yl)phenyl]methyl]piperazin-1-yl)pentyl]oxy]-2-(2,6-dioxopiperidin-3-yl)isoindole-1,3-dione; formic acid (5 mg, 10.9%) as a white solid. ¹H NMR (400 MHz, Methanol-d4) δ 9.54 (d, J=0.9 Hz, 1H), 8.69 (d, J=5.7 Hz, 1H), 8.52 (br s, 0.3H, FA),7.82 (d, J=8.3 Hz, 1H), 7.75 (s, 1H), 7.62 (dd, J=5.8, 0.9 Hz, 1H), 7.41 (d, J=2.2 Hz, 1H), 7.33 (dd, J=8.3, 2.3 Hz, 1H), 6.82 (s, 2H), 5.12 (dd, J=12.5, 5.4 Hz, 1H), 4.20 (t, J=6.2 Hz, 2H), 4.10 (s, 2H), 3.93 (s, 6H), 3.72 (s, 3H), 3.12-2.59 (m, 13H), 2.19-2.10 (m, 1H), 1.97-1.86 (m, 2H), 1.72-1.54 (m, 4H). LCMS (ESI) m/z: [M+H]+=737.40.

### Example 51—Preparation of 1-[[2,6-dimethoxy-4-(2-methyl-1-oxo-2,7-naphthyridin-4-yl)phenyl]methyl]-N-[2-(2-[[2-(2,6-dioxopiperidin-3-yl)-1,3-dioxoisoindol-4-yl]amino]ethoxy)ethyl]azetidine-3-sulfonamide (compound D45)

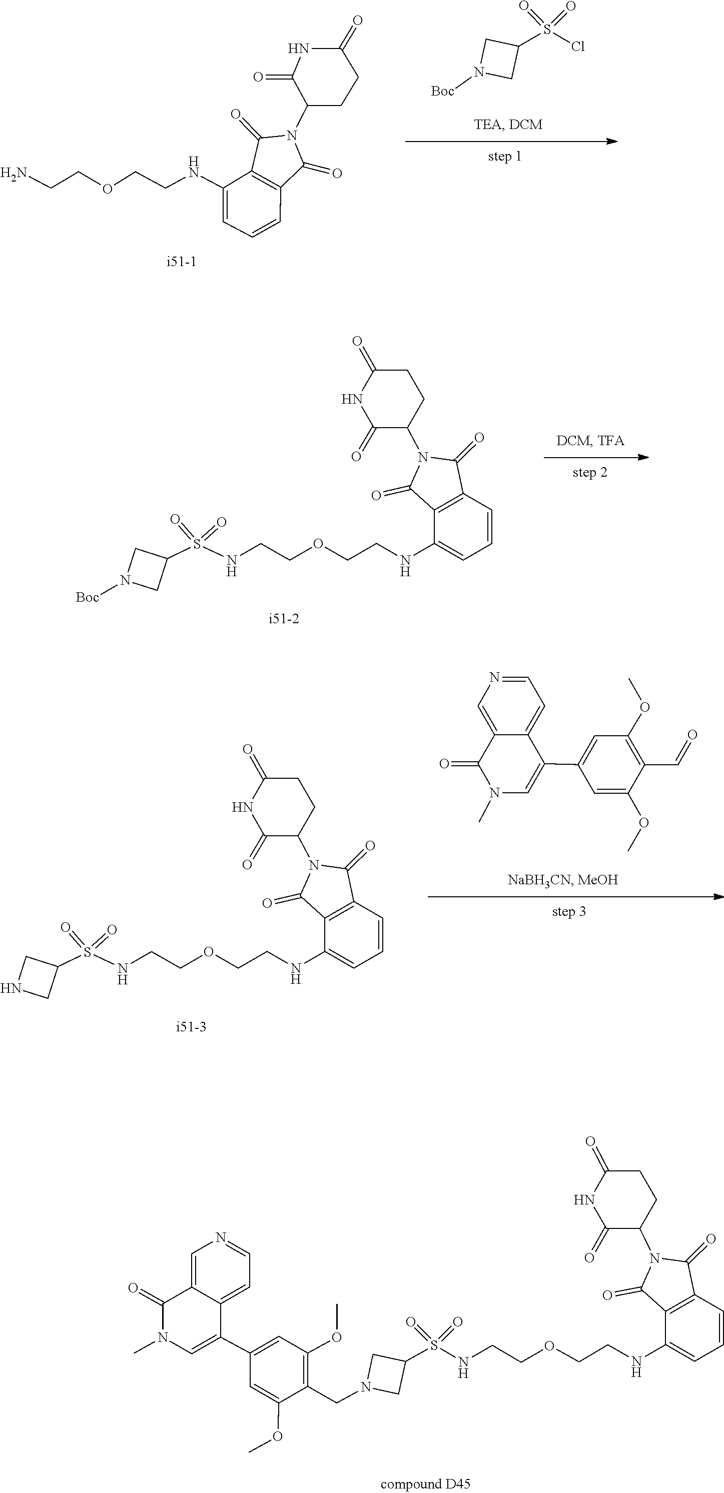 [SMILES: NCCOCCNc1cccc2c1C(=O)N(C1CCC(=O)NC1=O)C2=O ; CC(C)(C)OC(=O)N1CC(S(=O)(=O)Cl)C1 ; CC(C)(C)OC(=O)N1CC(S(=O)(=O)NCCOCCNc2cccc3c2C(=O)N(C2CCC(=O)NC2=O)C3=O)C1 ; O=C1CCC(N2C(=O)c3cccc(NCCOCCNS(=O)(=O)C4CNC4)c3C2=O)C(=O)N1 ; COc1cc(-c2cn(C)c(=O)c3cnccc23)cc(OC)c1C=O ; COc1cc(-c2cn(C)c(=O)c3cnccc23)cc(OC)c1CN1CC(S(=O)(=O)NCCOCCNc2cccc3c2C(=O)N(C2CCC(=O)NC2=O)C3=O)C1]

### Step 1: Preparation of tert-butyl 3-[[2-(2-[[2-(2,6-dioxopiperidin-3-yl)-1,3-dioxoisoindol-4-yl]amino]ethoxy) ethyl]sulfamoyl]azetidine-1-carboxylate (i51-2)

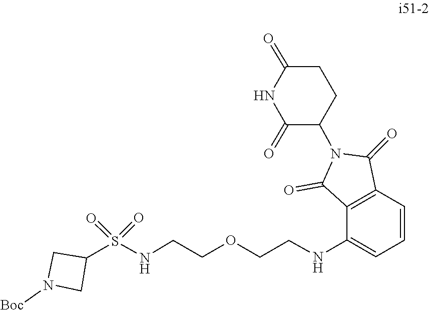 [SMILES: CC(C)(C)OC(=O)N1CC(S(=O)(=O)NCCOCCNc2cccc3c2C(=O)N(C2CCC(=O)NC2=O)C3=O)C1]

To a stirred solution of 4-[[2-(2-aminoethoxy)ethyl]amino]-2-(2,6-dioxopiperidin-3-yl)isoindole-1,3-dione (200.00 mg, 0.555 mmol, 1.00 equiv) and TEA (168.48 mg, 1.665 mmol, 3.00 equiv) in DCM (2 mL) was added tert-butyl 3-(chlorosulfonyl)azetidine-1-carboxylate (170.30 mg, 0.666 mmol, 1.20 equiv) at room temperature. The resulting mixture was stirred for 2 hours at room temperature. The resulting mixture was concentrated under vacuum. The residue was purified by silica gel column chromatography, eluted with CH₂Cl₂/MeOH (7:1) to afford tert-butyl 3-[[2-(2-[[2-(2,6-dioxopiperidin-3-yl)-1,3-dioxoisoindol-4-yl]amino]ethoxy)ethyl]sulfamoyl]azetidine-1-carboxylate (150 mg, 46.63%) as a yellow solid. LCMS (ESI) m/z: [M−H]+=580.20.

### Step 2: Preparation of N-[2-(2-[[2-(2,6-dioxopiperidin-3-yl)-1,3-dioxoisoindol-4-yl]amino]ethoxy)ethyl]azetidine-3-sulfonamide (i51-3)

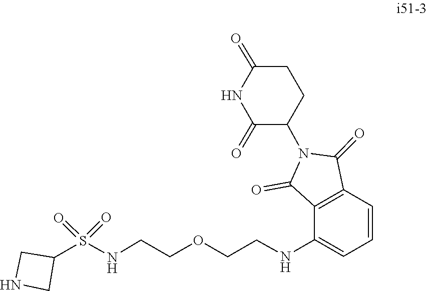 [SMILES: O=C1CCC(N2C(=O)c3cccc(NCCOCCNS(=O)(=O)C4CNC4)c3C2=O)C(=O)N1]

A solution of tert-butyl 3-[[2-(2-[[2-(2,6-dioxopiperidin-3-yl)-1,3-dioxoisoindol-4-yl]amino]ethoxy)ethyl]sulfamoyl]azetidine-1-carboxylate (100.00 mg, 0.173 mmol, 1.00 equiv) and TFA (1.00 mL) in DCM was stirred for 1 hour at room temperature. The resulting mixture was concentrated under vacuum. This resulted in N-[2-(2-[[2-(2,6-dioxopiperidin-3-yl)-1,3-dioxoisoindol-4-yl]amino]ethoxy)ethyl]azetidine-3-sulfonamide (75 mg, 90.66%) as a red oil. LCMS (ESI) m/z: [M−H]+=480.15.

### Step 3: Preparation of 1-[[2,6-dimethoxy-4-(2-methyl-1-oxo-2,7-naphthyridin-4-yl)phenyl]methyl]-N-[2-(2-[[2-(2,6-dioxopiperidin-3-yl)-1,3-dioxoisoindol-4-yl]amino]ethoxy)ethyl]azetidine-3-sulfonamide (compound D45)

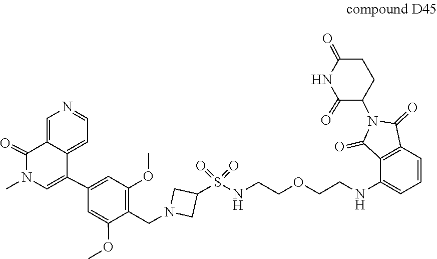 [SMILES: COc1cc(-c2cn(C)c(=O)c3cnccc23)cc(OC)c1CN1CC(S(=O)(=O)NCCOCCNc2cccc3c2C(=O)N(C2CCC(=O)NC2=O)C3=O)C1]

A solution of N-[2-(2-[[2-(2,6-dioxopiperidin-3-yl)-1,3-dioxoisoindol-4-yl]amino]ethoxy)ethyl]azetidine-3-sulfonamide (30.00 mg, 0.063 mmol, 1.00 equiv) and 2,6-dimethoxy-4-(2-methyl-1-oxo-2,7-naphthyridin-4-yl)benzaldehyde (26.38 mg, 0.081 mmol, 1.30 equiv) in DMF (2.00 mL) was stirred for 20 minutes at room temperature. Then NaBH(OAc)₃ (39.78 mg, 0.188 mmol, 3.00 equiv) was added to the reaction mixture. The resulting mixture was stirred for 1 hour at room temperature. The crude product was purified by Prep-HPLC (conditions: SunFire C18 OBD Prep Column, 100 Å, 5 μm, 19 mm×250 mm; mobile phase, Water (0.1% FA) and ACN (11% PhaseB up to 18% in 20 min, hold 18% in 3 minutes); Detector, UV). This resulted in 1-[[2,6-dimethoxy-4-(2-methyl-1-oxo-2,7-naphthyridin-4-yl)phenyl]methyl]-N-[2-(2-[[2-(2,6-dioxopiperidin-3-yl)-1,3-dioxoisoindol-4-yl]amino]ethoxy)ethyl]azetidine-3-sulfonamide (7.9 mg, 16.03%) as a green solid. ¹H NMR (400 MHz, Methanol-d4) δ 9.52 (s, 1H), 8.67 (d, J=5.8 Hz, 1H), 8.35 (br s, 0.3H, FA), 7.75 (s, 1H), 7.61 (dd, J=5.7, 0.9 Hz, 1H), 7.55 (dd, J=8.6, 7.1 Hz, 1H), 7.07 (dd, J=16.6, 7.8 Hz, 2H), 6.81 (s, 2H), 5.07 (d, J=12.3 Hz, 1H), 4.60 (s, 2H), 4.36 (s, 3H), 4.23 (d, J=7.7 Hz, 4H), 3.93 (s, 6H), 3.75 (t, J=5.2 Hz, 2H), 3.71 (s, 3H), 3.59 (t, J=5.2 Hz, 2H), 3.53 (t, J=5.2 Hz, 2H), 2.92-2.66 (m, 3H), 2.12 (ddd, J=12.7, 6.9, 3.9 Hz, 1H). LCMS (ESI) m/z: [M−H]⁺=788.26.

### Example 52—Preparation of 5-(4-(2-(2-((2,6-dimethoxy-4-(2-methyl-1-oxo-1,2-dihydro-2,7-naphthyridin-4-yl)benzyl)(methyl)amino)ethoxy)ethyl)piperazin-1-yl)-2-(2,6-dioxopiperidin-3-yl)isoindoline-1,3-dione formic acid (compound D46 formic acid)

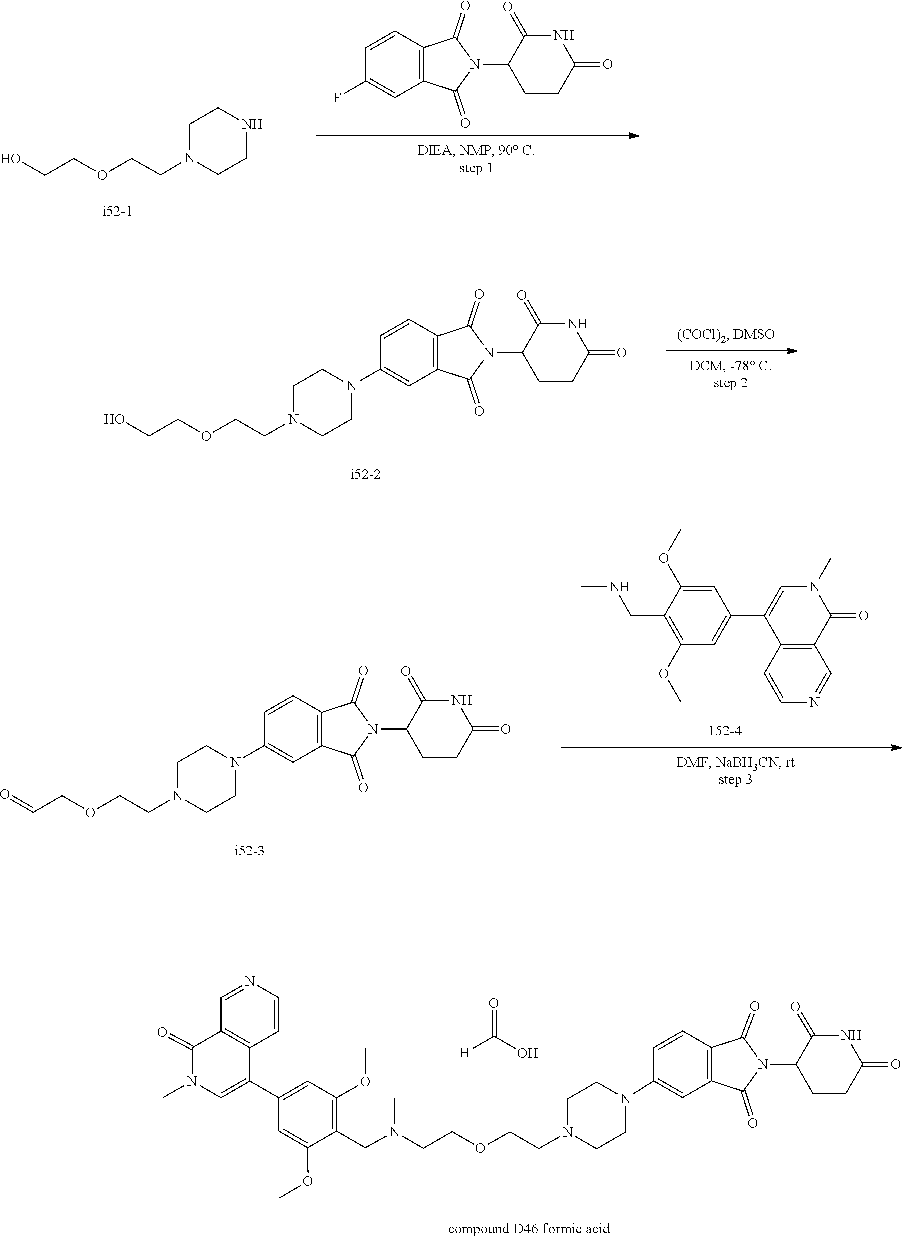

### Step 1: Preparation of 2-(2,6-dioxopiperidin-3-yl)-5-(4-(2-(2-hydroxyethoxy)ethyl)piperazin-1-yl)isoindoline-1,3-dione (i52-2)

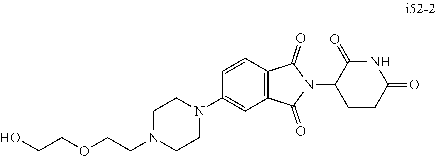 [SMILES: O=C1CCC(N2C(=O)c3ccc(N4CCN(CCOCCO)CC4)cc3C2=O)C(=O)N1]

To a solution of 2-[2-(piperazin-1-yl)ethoxy]ethan-1-ol (315.4 mg, 1.810 mmol, 1.00 equiv) and 2-(2,6-dioxopiperidin-3-yl)-5-fluoro-2,3-dihydro-1H-isoindole-1,3-dione (500.0 mg, 1.810 mmol, 1.00 equiv) in NMP (5 mL) was added DIEA (467.9 mg, 3.620 mmol, 2.00 equiv). The resulting mixture was stirred for 3 hours at 90° C. Without any additional work-up, the mixture was purified by reverse phase column, elution gradient 0% to 50% ACN in water to afford 2-(2,6-dioxopiperidin-3-yl)-5-[4-[2-(2-hydroxyethoxy)ethyl]piperazin-1-yl]-2,3-dihydro-1H-isoindole-1,3-dione (700.0 mg, 89.8%) as a yellow solid. LCMS (ESI) m/z: [M+H]⁺=431.

### Step 2: Preparation of 2-(2-(4-(2-(2,6-dioxopiperidin-3-yl)-1,3-dioxoisoindolin-5-yl)piperazin-1-yl)ethoxy)acetaldehyde (i52-3)

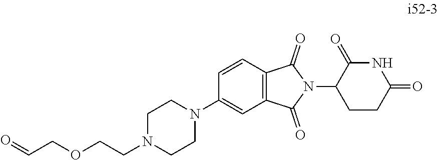 [SMILES: O=CCOCCN1CCN(c2ccc3c(c2)C(=O)N(C2CCC(=O)NC2=O)C3=O)CC1]

A solution of DMSO (54.5 mg, 0.697 mmol, 1.00 equiv) in DCM (6.00 mL) was added slowly to a stirred solution of oxalyl chloride (176.9 mg, 1.394 mmol, 2.00 equiv) in DCM (6.00 mL) at −78° C. under nitrogen atmosphere. After 30 minutes 2-(2,6-dioxopiperidin-3-yl)-5-[4-[2-(2-hydroxyethoxy)ethyl]piperazin-1-yl]isoindole-1,3-dione (300.0 mg, 0.697 mmol, 1.00 equiv) in DCM (6.00 mL) was added slowly. The resulting mixture was stirred for 2 hours at −78° C. and 1.5 hours at −55° C. Et₃N (0.48 mL, 4.787 mmol, 5.00 equiv) was added slowly at −60° C. After stirring for an additional 10 minutes, the reaction was allowed to warm to room temperature. The resulting mixture was quenched with saturated ammonium chloride aqueous solution (50 mL) and extracted with DCM (100 mL×3). The combined organic layers were washed with brine (50 mL), dried over anhydrous sodium sulfate, filtered, and concentrated in vacuo. The residue was purified by prep-TLC (EtOAc/PE=1:1) to afford 2-(2-(4-(2-(2,6-dioxopiperidin-3-yl)-1,3-dioxoisoindolin-5-yl)piperazin-1-yl)ethoxy)acetaldehyde (30.0 mg, 5.7%) as a yellow solid. LCMS (ESI) m/z: [M+H]⁺=429.

### Step 3: Preparation of 5-(4-(2-(2-((2,6-dimethoxy-4-(2-methyl-1-oxo-1,2-dihydro-2,7-naphthyridin-4-yl)benzyl)(methyl)amino)ethoxy)ethyl)piperazin-1-yl)-2-(2,6-dioxopiperidin-3-yl)isoindoline-1,3-dione formic acid (compound D46 formic acid)

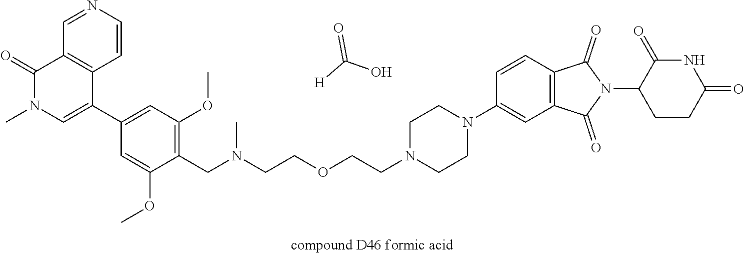 [SMILES: COc1cc(-c2cn(C)c(=O)c3cnccc23)cc(OC)c1CN(C)CCOCCN1CCN(c2ccc3c(c2)C(=O)N(C2CCC(=O)NC2=O)C3=O)CC1 ; O=CO]

To a mixture of 2-(2-[4-[2-(2,6-dioxopiperidin-3-yl)-1,3-dioxoisoindol-5-yl]piperazin-1-yl]eth oxy)acetaldehyde (30.0 mg, 0.070 mmol, 1.00 equiv) in DMF (2.00 mL) was added 4-[3,5-dim ethoxy-4-[(methylamino)methyl]phenyl]-2-methyl-2,7-naphthyridin-1-one (23.7 mg, 0.070 mmol, 1.00 equiv). The resulting mixture was stirred for 1 hour at room temperature, STAB (29.6 mg, 0.140 mmol, 2.00 equiv) was added. The resulting mixture was stirred for 1 hour at room temperature. The resulting mixture, without any additional wok-up, was purified by prep-HPLC (conditions: SunFire C₁₈ OBD Prep Column, 100 Å, 5 μm, 19 mm×250 mm; Mobile Phase A:Water (0.1% FA), Mobile Phase B:ACN; Flow rate:25 mL/minute; Gradient: 5% B to 30% B in 10 minutes; 254 nm; R_{T}: 8.82 minutes) to afford 5-(4-(2-(2-((2,6-dimethoxy-4-(2-methyl-1-oxo-1,2-dihydro-2,7-naphthyridin-4-yl)benzyl)(methyl)amino)ethoxy)ethyl)piperazin-1-yl)-2-(2,6-dioxopiperidin-3-yl)isoindoline-1,3-dione; formate (6.2 mg, 15.6%) as a light yellow solid. LCMS (ESI) m/z: [M+H]⁺=752.15. ¹H NMR (300 MHz, Methanol-d4) δ 9.47 (s, 1H), 8.64 (d, J=5.8 Hz, 1H), 8.57 (br s, 0.7H), 7.75 (s, 1H), 7.62 (dd, J=12.9, 7.1 Hz, 2H), 7.28 (d, J=2.3 Hz, 1H), 7.19 (d, J=9.0 Hz, 1H), 6.89 (s, 2H), 5.07 (dd, J=12.3, 5.4 Hz, 1H), 4.53 (s, 2H), 3.99 (s, 6H), 3.91 (t, J=4.7 Hz, 2H), 3.76 (t, J=5.1 Hz, 2H), 3.67 (s, 3H), 3.53-3.40 (m, 6H), 2.91 (s, 4H), 2.81-2.67 (m, 8H), 2.18-2.05 (m, 1H).

### Example 53—Preparation of 5-[[5-(9-[[2,6-dimethoxy-4-(2-methyl-1-oxo-2,7-naphthyridin-4-yl)phenyl]methyl]-1-oxa-4,9-diazaspiro[5.5]undecan-4-yl)pentyl]oxy]-2-(2,6-dioxopiperidin-3-yl)isoindole-1,3-dione formic acid (compound D47 formic acid)

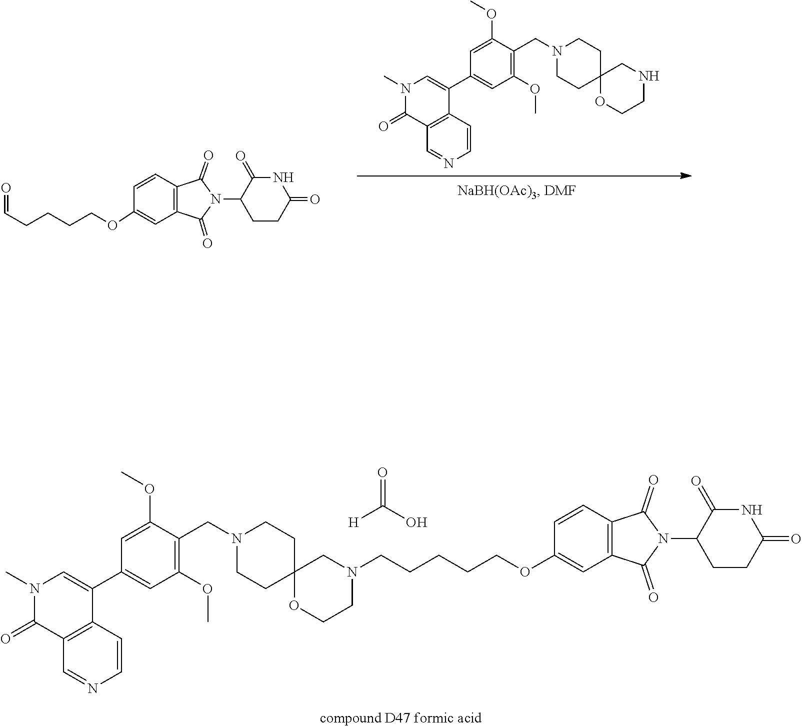

A solution of 5-[[2-(2,6-dioxopiperidin-3-yl)-1,3-dioxoisoindol-5-yl]oxy]pentanal (25 mg, 0.070 mmol, 1.00 equiv) and 4-(3,5-dimethoxy-4-[1-oxa-4,9-diazaspiro[5.5]undecan-9-ylmethyl]phenyl)-2-methyl-2,7-naphthyridin-1-one (32.4 mg, 0.070 mmol, 1.00 equiv) in DMF (0.8 mL) was stirred for 30 minutes at room temperature. NaBH(OAc)₃ (29.57 mg, 0.140 mmol, 2.00 equiv) was then added and the resulting mixture was stirred for 1 hour at room temperature. Without any additional work-up, the mixture was purified by Prep-HPLC (conditions: SunFire C18 OBD Prep Column, 100 Å, 5 μm, 19 mm×250 mm; Mobile Phase A: Water (0.1% FA), Mobile Phase B: ACN; Flow rate: 25 mL/minute; Gradient: 7% B to 20% B in 12 minutes; 254 nm; Rₜ: 11.57 minutes) to afford 5-[[5-(9-[[2,6-dimethoxy-4-(2-methyl-1-oxo-2,7-naphthyridin-4-yl)phenyl]methyl]-1-oxa-4,9-diazaspiro[5.5]undecan-4-yl)pentyl]oxy]-2-(2,6-dioxopiperidin-3-yl)isoindole-1,3-dione formic acid (7.9 mg, 13%) as a white solid. ¹H NMR (300 MHz, Methanol-d4) δ 9.55 (s, 1H), 8.69 (d, J=5.8 Hz, 1H), 8.50 (br s, 1H, FA), 7.86-7.75 (m, 2H), 7.63 (d, J=5.8 Hz, 1H), 7.40 (d, J=2.2 Hz, 1H), 7.32 (dd, J=8.3, 2.3 Hz, 1H), 6.88 (s, 2H), 5.11 (dd, J=12.3, 5.4 Hz, 1H), 4.42 (s, 2H), 4.19 (t, J=6.2 Hz, 2H), 3.98 (s, 6H), 3.76 (t, J=4.9 Hz, 2H), 3.72 (s, 3H), 3.44-3.35 (3H), 2.93-2.67 (m, 3H), 2.53-2.10 (m, 10H), 1.98-1.51 (m, 8H). LCMS (ESI) m/z: [M+H]+=807.50.

### Example 54—Preparation of N-(6-[4-[(dimethylamino)methyl]-3,5-dimethoxyphenyl]-3-methyl-[1,2,4]triazolo[4,3-a]pyridin-8-yl) acetamide formic acid (compound D48 formic acid)

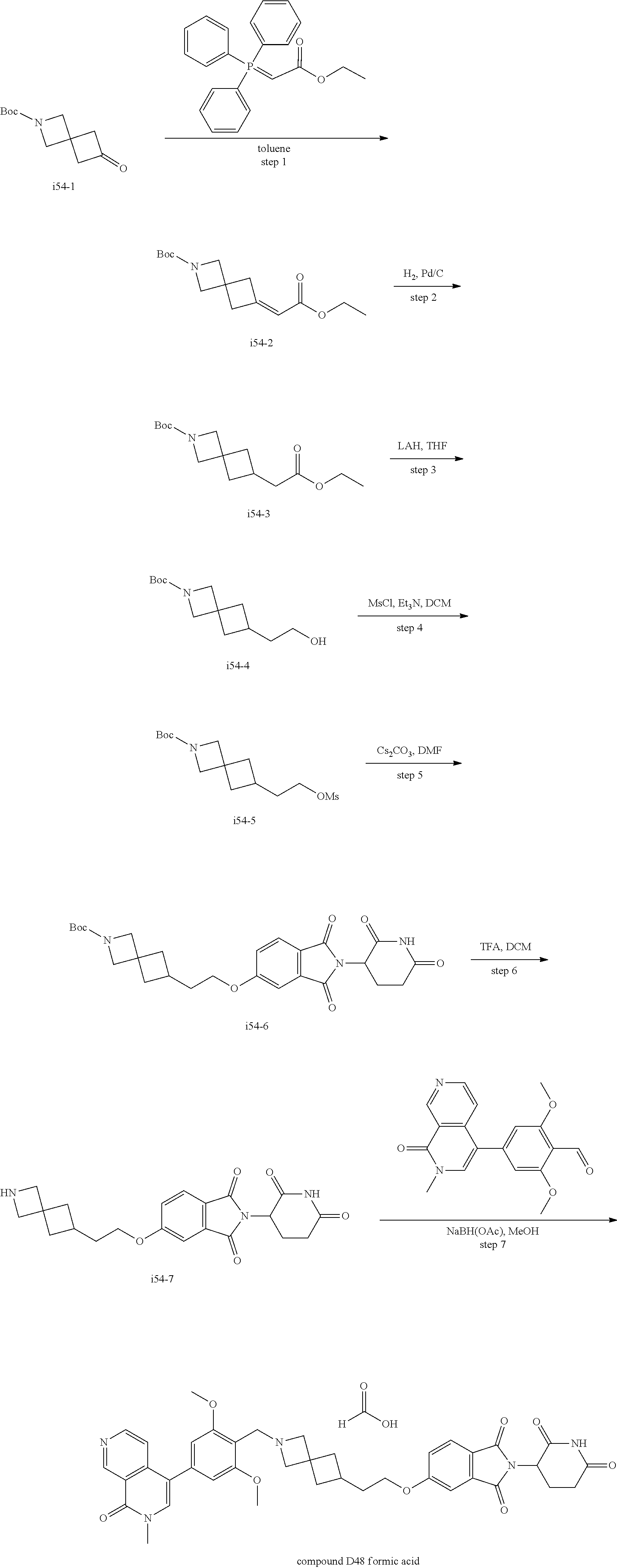

### Step 1: Preparation of tert-butyl 6-(2-ethoxy-2-oxoethylidene)-2-azaspiro[3.3]heptane-2-carboxylate (i54-2)

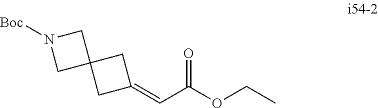 [SMILES: CCOC(=O)C=C1CC2(C1)CN(C(=O)OC(C)(C)C)C2]

A solution of tert-butyl 6-oxo-2-azaspiro[3.3]heptane-2-carboxylate (2.0 g, 9.467 mmol, 1.00 equiv) and ethyl2-(triphenyl-lambda5-phosphanylidene)acetate (3.63 g, 10.414 mmol, 1.10 equiv) in toluene was stirred for 4 hours at 80° C. under nitrogen atmosphere. The resulting mixture was washed with water (3×30 mL). The resulting mixture was concentrated under vacuum. The residue was purified by silica gel column chromatography, eluted with PE/EtOAc (1:1) to afford tert-butyl 6-(2-ethoxy-2-oxoethylidene)-2-azaspiro[3.3]heptane-2-carboxylate (2.51 g, 94.09%) as a light yellow oil. LCMS (ESI) m/z: [M+H]+=282.

### Step 2: Preparation of tert-butyl 6-(2-ethoxy-2-oxoethyl)-2-azaspiro[3.3]heptane-2-carboxylate (i54-3)

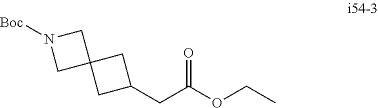 [SMILES: CCOC(=O)CC1CC2(C1)CN(C(=O)OC(C)(C)C)C2]

To a solution of tert-butyl 6-(2-ethoxy-2-oxoethylidene)-2-azaspiro[3.3]heptane-2-carboxylate (2506.00 mg, 8.907 mmol, 1.00 equiv) in MeOH (25 mL) was added Pd/C (10%, 1 g) under nitrogen atmosphere. The mixture was hydrogenated at room temperature for 1 day under hydrogen atmosphere using a hydrogen balloon, filtered through a Celite pad, and concentrated under reduced pressure afford tert-butyl 6-(2-ethoxy-2-oxoethyl)-2-azaspiro[3.3]heptane-2-carboxylate (2100.00 mg, 81.4%) as a light yellow oil. LCMS (ESI) m/z: [M+H]+=284.

### Step 3: Preparation of tert-butyl 6-(2-hydroxyethyl)-2-azaspiro[3.3]heptane-2-carboxylate (i54-4)

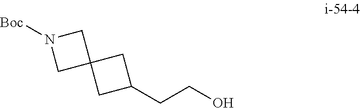 [SMILES: CC(C)(C)OC(=O)N1CC2(CC(CCO)C2)C1]

To a stirred solution of tert-butyl 6-(2-ethoxy-2-oxoethyl)-2-azaspiro[3.3]heptane-2-carboxylate (1.0 g, 3.529 mmol, 1.00 equiv) in THF (20 ml) was added LAH (267.88 mg, 7.058 mmol, 2 equiv) in portions at 0° C. under nitrogen atmosphere. The reaction was quenched with Na₂SO₄·10H₂O at room temperature. The resulting mixture was filtered. The filter cake was washed with MeOH (3×20 mL). The filtrate was concentrated under reduced pressure. The crude product (537.00 mg, 63.0%) was used in the next step directly without further purification. LCMS (ESI) m/z: [M+H]+=242.

### Step 4: Preparation of tert-butyl 6-(2-((methylsulfonyl)oxy)ethyl)-2-azaspiro[3.3]heptane-2-carboxylate (i54-5)

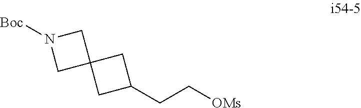 [SMILES: CC(C)(C)OC(=O)N1CC2(CC(CCOS(C)(=O)=O)C2)C1]

A solution of tert-butyl 6-(2-hydroxyethyl)-2-azaspiro[3.3]heptane-2-carboxylate (537.00 mg, 2.225 mmol, 1.00 equiv), Et₃N (450.33 mg, 4.450 mmol, 2.00 equiv), and MsCl (280.38 mg, 2.448 mmol, 1.10 equiv) in DCM (5 mL) was stirred for 3 hours at room temperature under nitrogen atmosphere. The resulting mixture was extracted with EtOAc (1×20 mL). The combined organic layers were washed with water (3×10 mL), dried over anhydrous Na₂SO₄, and concentrated. The residue was purified by silica gel column chromatography, eluted with CH₂Cl₂/MeOH (0% to 18%) to afford tert-butyl 6-[2-(methanesulfonyloxy)ethyl]-2-azaspiro[3.3]heptane-2-carboxylate (593 mg, 83.43%) as a white solid. LCMS (ESI) m/z: [M+H]+=320

### Step 5: Preparation of tert-butyl 6-(2-((2-(2,6-dioxopiperidin-3-yl)-1,3-dioxoisoindolin-5-yl)oxy)ethyl)-2-azaspiro[3.3]heptane-2-carboxylate (i54-6)

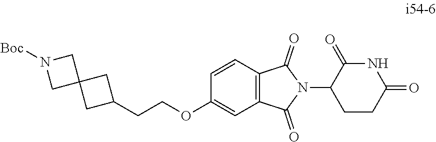 [SMILES: CC(C)(C)OC(=O)N1CC2(CC(CCOc3ccc4c(c3)C(=O)N(C3CCC(=O)NC3=O)C4=O)C2)C1]

A solution of tert-butyl 6-[2-(methanesulfonyloxy)ethyl]-2-azaspiro[3.3]heptane-2-carboxylate (320.00 mg, 1.002 mmol, 1.00 equiv),Cs₂CO₃ (652.82 mg, 2.004 mmol, 2.00 equiv), and 2-(2,6-dioxopiperidin-3-yl)-5-hydroxyisoindole-1,3-dione (274.73 mg, 1.002 mmol, 1.00 equiv) in DMF (3 mL) was stirred for 15 hours at room temperature under nitrogen atmosphere. The resulting mixture was extracted with EtOAc (1×100 mL). The combined organic layers was washed with water (3×100 mL), dried over anhydrous Na₂SO₄. After filtration, the filtrate was concentrated under reduced pressure to afford tert-butyl6-(2-[[2-(2,6-dioxopiperidin-3-yl)-1,3-dioxoisoindol-5-yl]oxy]ethyl)-2-azaspiro[3.3]heptane-2-carboxylate (265.0 Omg, 53.2%) as a yellow oil. LCMS (ESI) m/z: [M+H]+=498.

### Step 6: Preparation of 5-(2-(2-azaspiro[3.3]heptan-6-yl)ethoxy)-2-(2,6-dioxopiperidin-3-yl)isoindoline-1,3-dione (i54-7)

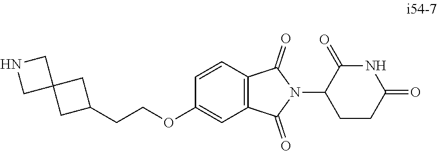 [SMILES: O=C1CCC(N2C(=O)c3ccc(OCCC4CC5(CNC5)C4)cc3C2=O)C(=O)N1]

A solution of tert-butyl 6-(2-[[2-(2,6-dioxopiperidin-3-yl)-1,3-dioxoisoindol-5-yl]oxy]ethyl)-2-azaspiro[3.3]heptane-2-carboxylate (265.00 mg, 0.533 mmol, 1.00 equiv) and TFA (2.5 mL) in DCM (5.0 mL) was stirred for 1.5 hours at room temperature under nitrogen atmosphere. The resulting mixture was concentrated under reduced pressure. The residue was purified by Prep-TLC (CH₂Cl₂/EtOAc 1:1) to afford 5-(2-[2-azaspiro[3.3]heptan-6-yl]ethoxy)-2-(2,6-dioxopiperidin-3-yl)isoindole-1,3-dione (200 mg, 94.48%) as a yellow oil. LCMS (ESI) m/z: [M+H]+=398.

### Step 7: Preparation of 5-(2-(2-(2,6-dimethoxy-4-(2-methyl-1-oxo-1,2-dihydro-2,7-naphthyridin-4-yl) benzyl)-2-azaspiro[3.3]heptan-6-yl)ethoxy)-2-(2,6-dioxopiperidin-3-yl)isoindoline-1,3-dione formic acid (compound D48 formic acid)

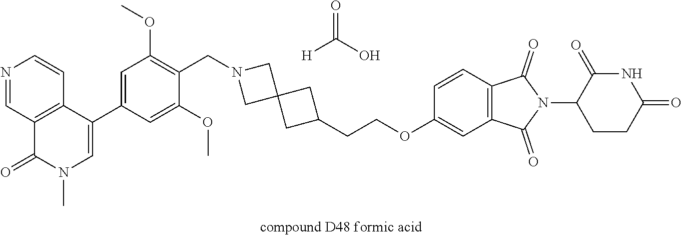

A solution of 5-(2-[2-azaspiro[3.3]heptan-6-yl]ethoxy)-2-(2,6-dioxopiperidin-3-yl)isoindole-1,3-dione (51.00 mg, 0.128 mmol, 1.00 equiv) in MeOH (1 mL) was treated with 2,6-dimethoxy-4-(2-methyl-1-oxo-2,7-naphthyridin-4-yl)benzaldehyde (41.62 mg, 0.128 mmol, 1.00 equiv) for 20 minutes at room temperature under nitrogen atmosphere followed by the addition of NaBH₃CN (16.13 mg, 0.257 mmol, 2.00 equiv) in portions at room temperature. The residue was purified by reverse flash chromatography (conditions: column, C18 silica gel; mobile phase, MeOH in water, 10% to 50% gradient in 10 minutes; detector, UV 254 nm). This resulted in 5-(2-(2-(2,6-dimethoxy-4-(2-methyl-1-oxo-1,2-dihydro-2,7-naphthyridin-4-yl)benzyl)-2-azaspiro[3.3]heptan-6-yl)ethoxy)-2-(2,6-dioxopiperidin-3-yl)iso indoline-1,3-dione formic acid (2.4 mg, 2.2%) as a yellow solid. ¹H NMR (300 MHz, Methanol-d4) δ 9.54 (s, 1H), 8.68 (d, J=5.7 Hz, 1H), 8.56 (brs, 1.1H, FA), 7.77 (s, 1H), 7.68-7.56 (m, 2H), 7.13 (d, J=2.2 Hz, 1H), 7.05 (dd, J=8.2, 2.2 Hz, 1H), 6.85 (s, 2H), 5.10 (dd, J=12.9, 5.5 Hz, 1H), 4.40 (s, 2H), 4.21-4.12 (m, 2H), 4.05 (s, 2H), 3.96 (s, 6H), 3.79-3.70 (m, 5H), 2.95-2.84 (m, 2H), 2.75-2.59 (m, 1H), 2.49-2.36 (m, 2H), 2.27-2.06 (m, 2H), 2.05-1.92 (m, 2H), 1.72-1.54 (m, 2H). LCMS (ESI) m/z: [M+H]+=706.50.

### Example 55—Preparation of 5-[2-(9-[[2,6-dimethoxy-4-(2-methyl-1-oxo-2,7-naphthyridin-4-yl)phenyl]methyl]-1-oxa-4,9-diazaspiro[5.5]undecan-4-yl)ethoxy]-2-(2,6-dioxopiperidin-3-yl)isoindole-1,3-dione (compound D49)

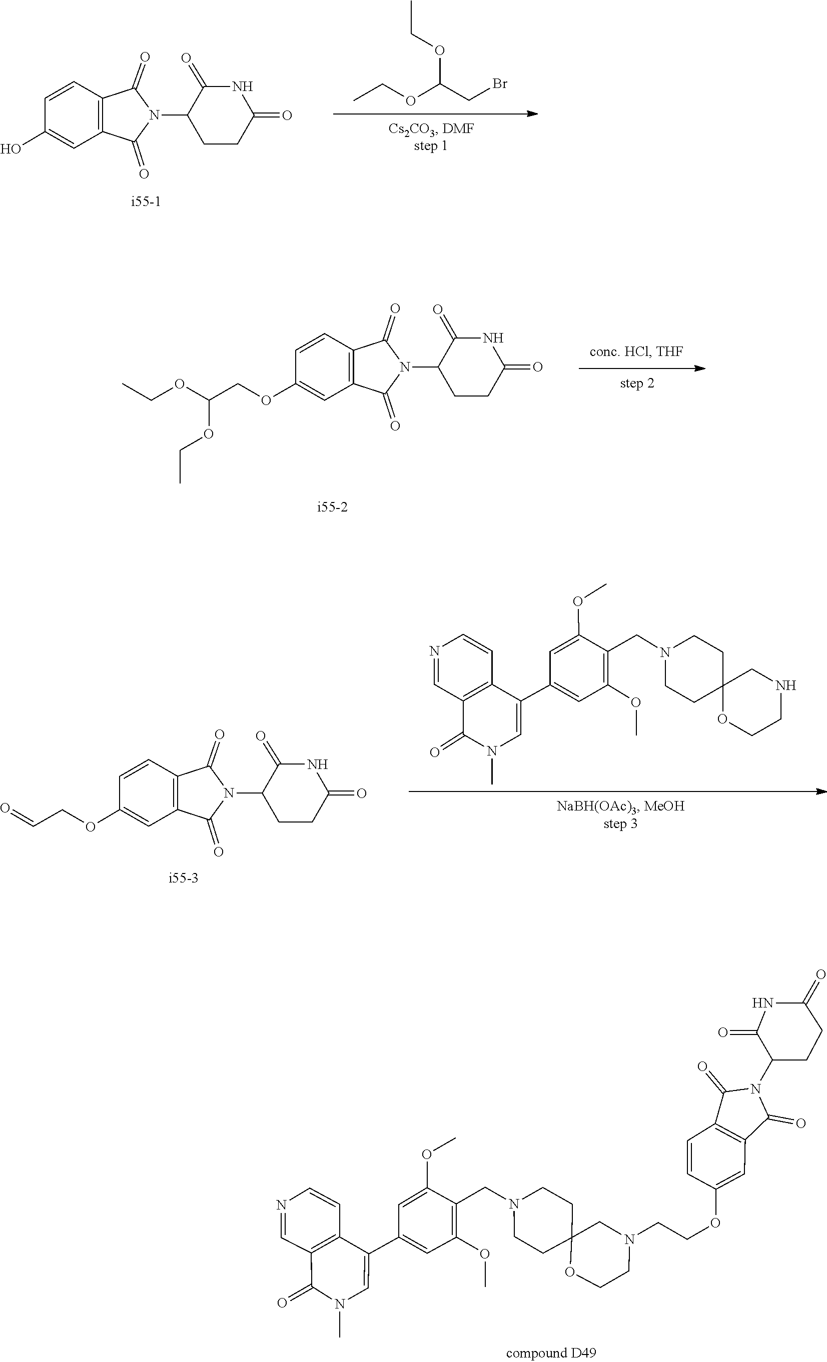

### Step 1: Preparation of 5-(2,2-Diethoxyethoxy)-2-(2,6-dioxopiperidin-3-yl)isoindole-1,3-dione (i55-2)

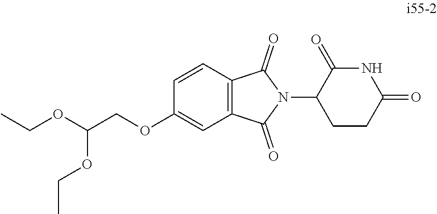 [SMILES: CCOC(COc1ccc2c(c1)C(=O)N(C1CCC(=O)NC1=O)C2=O)OCC]

To a stirred solution of 2-(2,6-dioxopiperidin-3-yl)-5-hydroxyisoindole-1,3-dione (500.00 mg, 1.823 mmol, 1.00 equiv) and Cs₂CO₃ (980.20 mg, 3.008 mmol, 3 equiv) in DMF (10.00 mL) was added 2-bromo-1,1-diethoxyethane (538.97 mg, 2.735 mmol, 1.5 equiv). The mixture was stirred at 80° C. for 16 hours. The mixture was acidified to pH 6 with HCl (aq.). The mixture was diluted with water (40 mL) and extracted with EtOAc/DCM (60 mL×3). The organic layers were combined and dried over anhydrous sodium sulfate, filtered, and concentrated to give a crude product. The residue was purified by Prep-TLC (PE/EtOAc 1:1) to afford 5-(2,2-diethoxyethoxy)-2-(2,6-dioxopiperidin-3-yl)isoindole-1,3-dione(110 mg, 15.45%) as a yellow solid. LCMS (ESI) m/z: [M+H]⁺=391.

### Step 2: Preparation of 2-[[2-(2,6-Dioxopiperidin-3-yl)-1,3-dioxoisoindol-5-yl]oxy]acetaldehyde (i55-3)

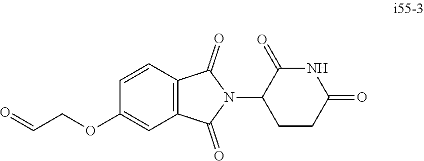 [SMILES: O=CCOc1ccc2c(c1)C(=O)N(C1CCC(=O)NC1=O)C2=O]

To a stirred solution of 5-(2,2-diethoxyethoxy)-2-(2,6-dioxopiperidin-3-yl)isoindole-1,3-dione (100.00 mg, 0.256 mmol, 1.00 equiv) in THE (2.00 mL) was added HCl (4 M) (2.00 mL). The mixture was stirred at room temperature for 4 hours. The mixture was diluted with water (20 mL) and extracted with EtOAc/DCM (30 mL×3). The organic layers were combined and dried over anhydrous sodium sulfate, filtered, and concentrated to give a crude product. This resulted in 2-[[2-(2,6-dioxopiperidin-3-yl)-1,3-dioxoisoindol-5-yl]oxy]aceta Idehyde (95 mg, crude) as a white solid. LCMS (ESI) m/z: [M+H]+=317.

### Step 3: Preparation of 5-[2-(9-[[2,6-Dimethoxy-4-(2-methyl-1-oxo-2,7-naphthyridin-4-yl)phenyl]methyl]-1-oxa-4,9-diazaspiro[5.5]undecan-4-yl)ethoxy]-2-(2,6-dioxopiperidin-3-yl)isoindole-1,3-dione (compound D49)

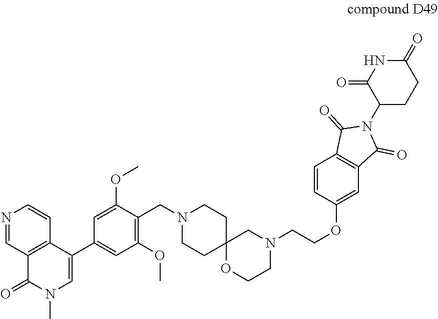 [SMILES: COc1cc(-c2cn(C)c(=O)c3cnccc23)cc(OC)c1CN1CCC2(CC1)CN(CCOc1ccc3c(c1)C(=O)N(C1CCC(=O)NC1=O)C3=O)CCO2]

To a stirred solution of 2-[[2-(2,6-dioxopiperidin-3-yl)-1,3-dioxoisoindol-5-yl]oxy]acetaldehyde (60.00 mg, 0.190 mmol, 1.00 equiv) and 4-(3,5-dimethoxy-4-[1-oxa-4,9-diazaspiro[5.5]undecan-9-ylmethyl]phenyl)-2-methyl-2,7-naphthyridin-1-one (88.13 mg, 0.190 mmol, 1.00 equiv) in DMF (1.50 mL) was added NaBH(OAc)₃ (80.42 mg, 0.379 mmol, 2.00 equiv). The mixture was stirred at room temperature for 2 hours. Without any additional work-up, the mixture was purified by prep-HPLC (conditions: Xcelect CSH F-pheny OBD Column, 19*250 mm, 5 μm; Mobile Phase A: Water (0.05% TFA), Mobile Phase B:ACN; Flow rate:25 mL/minute; Gradient:11 B to 19 B in 12 minutes; 254/220 nm; RT:10.70 minutes) to give 5-[2-(9-[[2,6-dimethoxy-4-(2-methyl-1-oxo-2,7-naphthyridin-4-yl)phenyl]methyl]-1-oxa-4,9-diazaspiro[5.5]undecan-4-yl)ethoxy]-2-(2,6-dioxopiperidin-3-yl)isoindole-1,3-dione(8.2 mg, 5.5 2%) as a yellow solid. ¹H NMR (300 MHz, Methanol-d4) δ 9.59 (s, 1H), 8.71 (s, 1H), 7.96 (d, J=7.2 Hz, 1H), 7.82 (d, J=11.3 Hz, 1H), 7.71 (t, J=8.8 Hz, 1H), 7.24-7.05 (m, 2H), 6.85 (d, J=18.8 Hz, 2H), 5.32-5.16 (m, 1H), 4.43 (s, 2H), 4.20 (s, 2H), 3.97 (s, 7H), 3.90 (s, 1H), 3.75 (s, 3H), 3.59-3.38 (m, 4H), 3.31-3.12 (m, 5H), 3.05-2.86 (m, 2H), 2.82-2.63 (m, 1H), 2.47-1.84 (m, 5H). LCMS (ESI) m/z: [M+H]+=765.45.

### Example 56—Preparation of 5-(4-(9-(2,6-dimethoxy-4-(2-methyl-1-oxo-1,2-dihydro-2,7-naphthyridin-4-yl)benzyl)-1-oxa-4,9-diazaspiro[5.5]undecan-4-yl)butoxy)-2-(2,6-dioxopiperidin-3-yl)isoindoline-1,3-dione (compound D50)

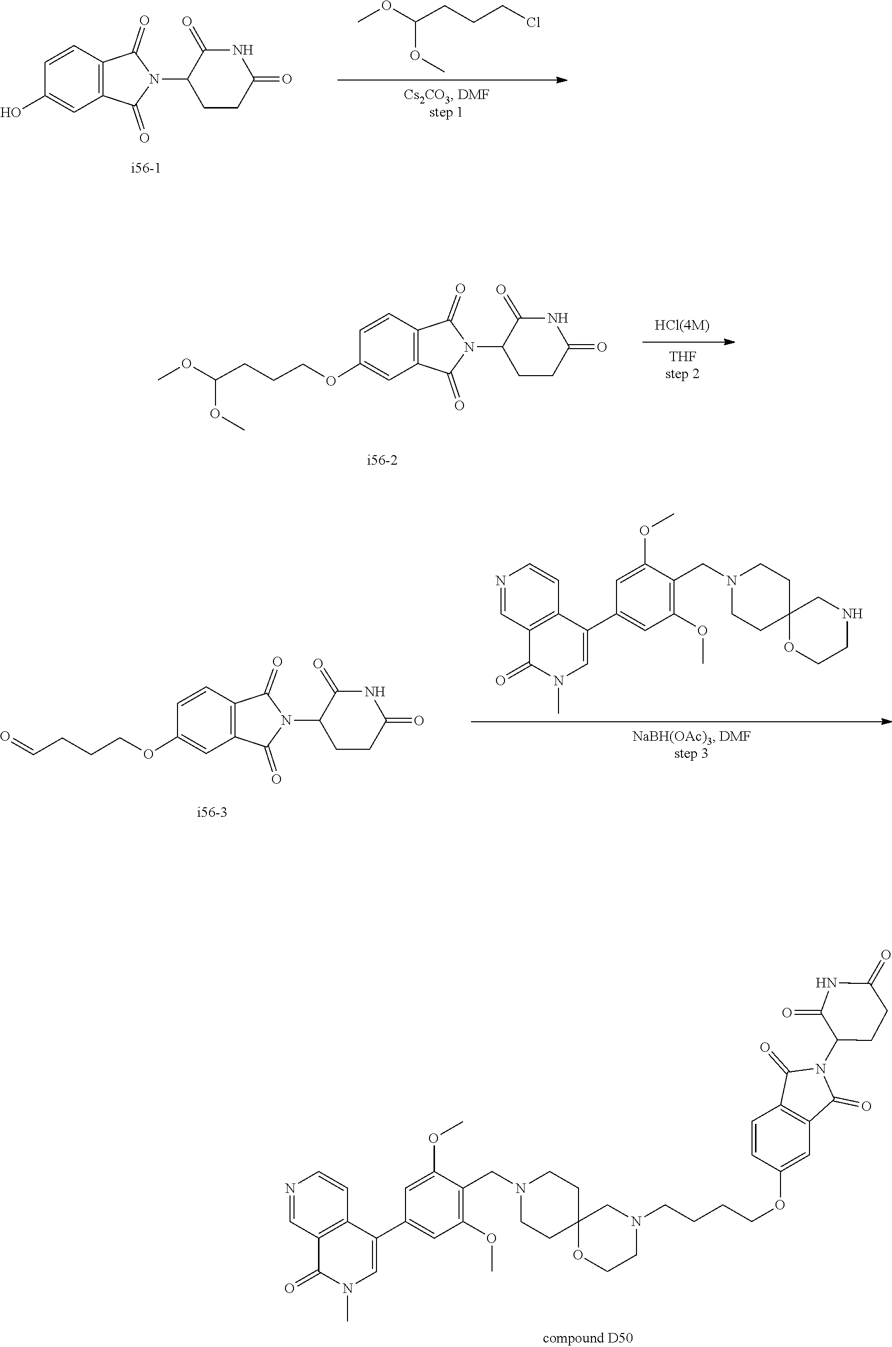

### Step 1: Preparation of 5-(4,4-dimethoxybutoxy)-2-(2,6-dioxopiperidin-3-yl)isoindoline-1,3-dione (i56-2)

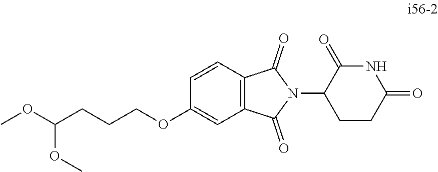 [SMILES: COC(CCCOc1ccc2c(c1)C(=O)N(C1CCC(=O)NC1=O)C2=O)OC]

To a solution of 2-(2,6-dioxopiperidin-3-yl)-5-hydroxyisoindole-1,3-dione (500.00 mg, 1.823 mmol, 1.00 equiv) and 4-chloro-1,1-dimethoxybutane (278.27 mg, 1.823 mmol, 1 equiv) in DMF (7.00 mL) was added K₂CO₃ (755.96 mg, 5.470 mmol, 3 equiv). The resulting solution was stirred at 80° C. for 12 hours. The resulting mixture was extracted with EA (50 mL×2). The combined organic layers were washed with saturated NaCl (50 mL) and dried over anhydrous Na₂SO₄. After filtration, the filtrate was concentrated under reduced pressure. The residue was purified by silica gel column chromatography, eluted with EA/PE (100:0) to afford 5-(4,4-dimethoxybutoxy)-2-(2,6-dioxopiperidin-3-yl)isoindole-1,3-dione (43.6 mg, 6.13%) as an off-white solid. LCMS (ESI) m/z: [M+H]⁺=391.

### Step 2: Preparation of 4-((2-(2,6-dioxopiperidin-3-yl)-1,3-dioxoisoindolin-5-yl)oxy)butanal (i56-3)

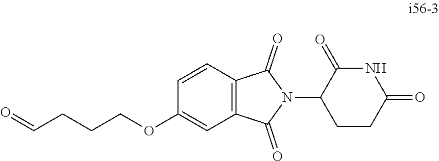 [SMILES: O=CCCCOc1ccc2c(c1)C(=O)N(C1CCC(=O)NC1=O)C2=O]

A solution of 5-(4,4-dimethoxybutoxy)-2-(2,6-dioxopiperidin-3-yl)isoindole-1,3-dione (43.60 mg, 0.112 mmol, 1.00 equiv) and HCl (1.00 mL, 4M) in THE (1.00 mL) was stirred at 25° C. for 1 hour. The resulting mixture was extracted with EA (50 mL×2). The combined organic layers were washed with saturated NaCl (50 mL) and dried over anhydrous Na₂SO₄. After filtration, the filtrate was concentrated under reduced pressure to afford 4-[[2-(2,6-dioxopiperidin-3-yl)-1,3-dioxoisoindol-5-yl]oxy]butanal (34.6 mg, 89.98%) as an off-white solid. LCMS (ESI) m/z: [M+H]+=345.

### Step 3: Preparation of 5-(4-(9-(2,6-dimethoxy-4-(2-methyl-1-oxo-1,2-dihydro-2,7-naphthyridin-4-yl)benzyl)-1-oxa-4,9-diazaspiro[5.5]undecan-4-yl)butoxy)-2-(2,6-dioxopiperidin-3-yl)isoindoline-1,3-dione (compound D50)

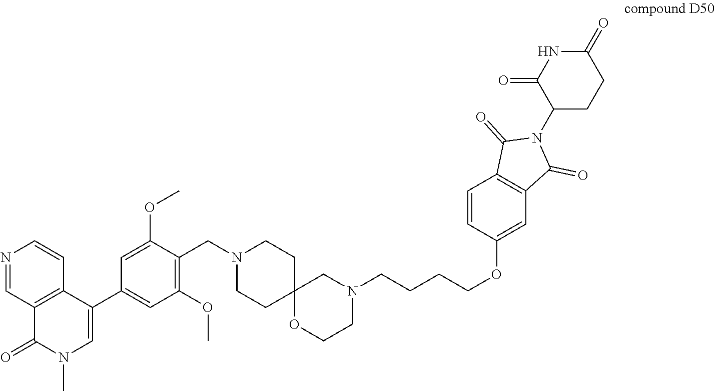 [SMILES: COc1cc(-c2cn(C)c(=O)c3cnccc23)cc(OC)c1CN1CCC2(CC1)CN(CCCCOc1ccc3c(c1)C(=O)N(C1CCC(=O)NC1=O)C3=O)CCO2]

To a solution of 4-[[2-(2,6-dioxopiperidin-3-yl)-1,3-dioxoisoindol-5-yl]oxy]butanal (34.00 mg, 0.099 mmol, 1.00 equiv) and 4-(3,5-dimethoxy-4-[1-oxa-4,9-diazaspiro[5.5]undecan-9-ylmethyl]phenyl)-2-methyl-2,7-naphthyridin-1-one (45.87 mg, 0.099 mmol, 1 equiv) in DMF (1.00 mL) was added NaBH(OAc)₃ (41.86 mg, 0.197 mmol, 2 equiv). The resulting solution was stirred at 25° C. for 1 hour. The mixture was purified by prep-HPLC (conditions: Xselect CSH F-Phenyl OBD Column 19*150 mm 5 μm; Mobile Phase A: Water (0.05% TFA), Mobile Phase B:ACN; Flow rate:25 mL/minute; Gradient:10 B to 19 B in 15 minutes; 254/220 nm; R_{T}: 14.53 minutes) to afford 5-[4-(9-[[2,6-dimethoxy-4-(2-methyl-1-oxo-2,7-naphthyridin-4-yl)phenyl]methyl]-1-oxa-4,9-diazaspiro[5.5]undecan-4-yl)butoxy]-2-(2,6-dioxopiperidin-3-yl)isoindole-1,3-dione (14 mg, 17.88%) as an off-white solid. ¹H NMR (300 MHz, Methanol-d4) δ 9.57 (s, 1H), 8.70 (d, J=6.0 Hz, 1H), 7.87 (s, 1H), 7.74 (d, J=7.7 Hz, 2H), 7.27-7.14 (m, 2H), 6.89 (s, 2H), 5.16 (dd, J=12.8, 5.5 Hz, 1H), 4.45 (s, 2H), 4.09-4.01 (m, 2H), 3.98 (s, 6H), 3.89 (t, J=6.4 Hz, 2H), 3.73 (s, 3H), 3.57-3.48 (m, 2H), 3.28-3.17 (m, 4H), 2.98-2.87 (m, 2H), 2.85-2.59 (m, 2H), 2.41-2.25 (m, 1H), 2.23-2.07 (m, 2H), 2.05-1.90 (m, 2H), 1.89-1.59 (m, 5H). LCMS (ESI) m/z: [M+H]+=793.3.

### Example 57—Preparation of 5-[2-[4-([[2,6-dimethoxy-4-(2-methyl-1-oxo-2,7-naphthyridin-4-yl)phenyl]methyl](methyl)amino)piperidin-1-yl]ethoxy]-2-(2,6-dioxopiperidin-3-yl)isoindole-1,3-dione; formic acid (compound D51 formic acid)

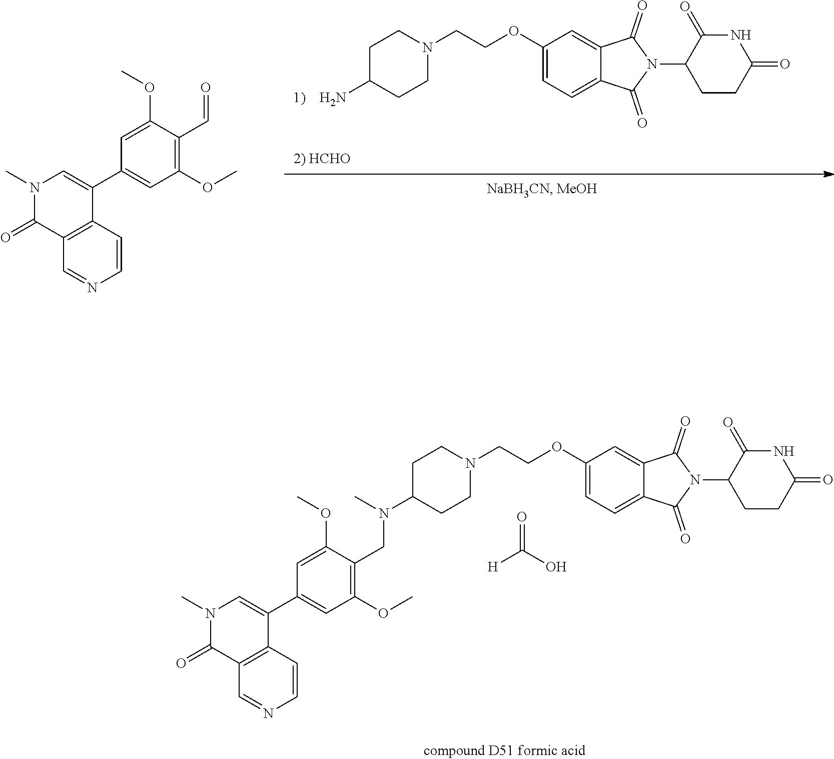

To a solution of 2,6-dimethoxy-4-(2-methyl-1-oxo-2,7-naphthyridin-4-yl)benzaldehyde (30.00 mg, 0.092 mmol, 1.00 equiv) and 5-[2-(4-aminopiperidin-1-yl)ethoxy]-2-(2,6-dioxopiperidin-3-yl)isoindole-1,3-dione (37.04 mg, 0.092 mmol, 1.00 equiv) in MeOH (1 mL) was stirred for 3 hours at room temperature under nitrogen atmosphere. To the above mixture was added NaBH₃CN (11.63 mg, 0.185 mmol, 2.00 equiv), and the reaction was stirred for additional 1 hour at room temperature. To the above mixture was added HCHO (27.77 mg, 0.925 mmol, 10.00 equiv), and the reaction was stirred for 1 hour at room temperature under nitrogen atmosphere. Then NaBH₃CN (11.63 mg, 0.185 mmol, 2.00 equiv) was added. The mixture was stirred for overnight at room temperature under nitrogen atmosphere. The crude product (40 mg) was purified by Prep-HPLC (conditions: Gemini-NX C18 AXAI Packed column, 21.2*150 mm 5 μm; Mobile Phase A: Water (0.1% FA), Mobile Phase B:ACN; Flow rate:25 mL/minute; Gradient:5 B to 17 B in 9 minutes; 254-220 nm; R_{T}: 8.30 minutes) to afford 5-[2-[4-([[2,6-dimethoxy-4-(2-methyl-1-oxo-2,7-naphthyridin-4-yl)phenyl]methyl](methyl)amino)piperidin-1-yl]ethoxy]-2-(2,6-dioxopiperidin-3-yl)iso indole-1,3-dione formic acid (7.8 mg) as a white solid. ¹H NMR (300 MHz, DMSO-d6) δ 1.55 (2H, d), 1.77 (2H, d), 2.03 (3H, d), 2.16 (3H, s), 2.44 (3H, d), 2.73 (2H, s), 2.88-3.08 (3H, m), 3.61 (5H, s), 3.80 (6H, s), 4.30 (2H, s), 5.12 (1H, m), 6.72 (2H, s), 7.38 (1H, m), 7.48 (1H, d), 7.57 (1H, d), 7.80-7.90 (2H, m), 8.23 (1H, s), 8.72 (1H, d), 9.45 (1H, s), 11.12 (1H, s). LCMS (ESI) m/z: [M+H]+=723.40.

### Example 58—Preparation of 5-((1-(3-((2,6-dimethoxy-4-(2-methyl-1-oxo-1,2-dihydro-2,7-naphthyridin-4-yl)benzyl) (methyl)amino) propyl)piperidin-4-yl)oxy)-2-(2,6-dioxopiperidin-3-yl)isoindoline-1,3-dione formic acid (compound D52 formic acid)

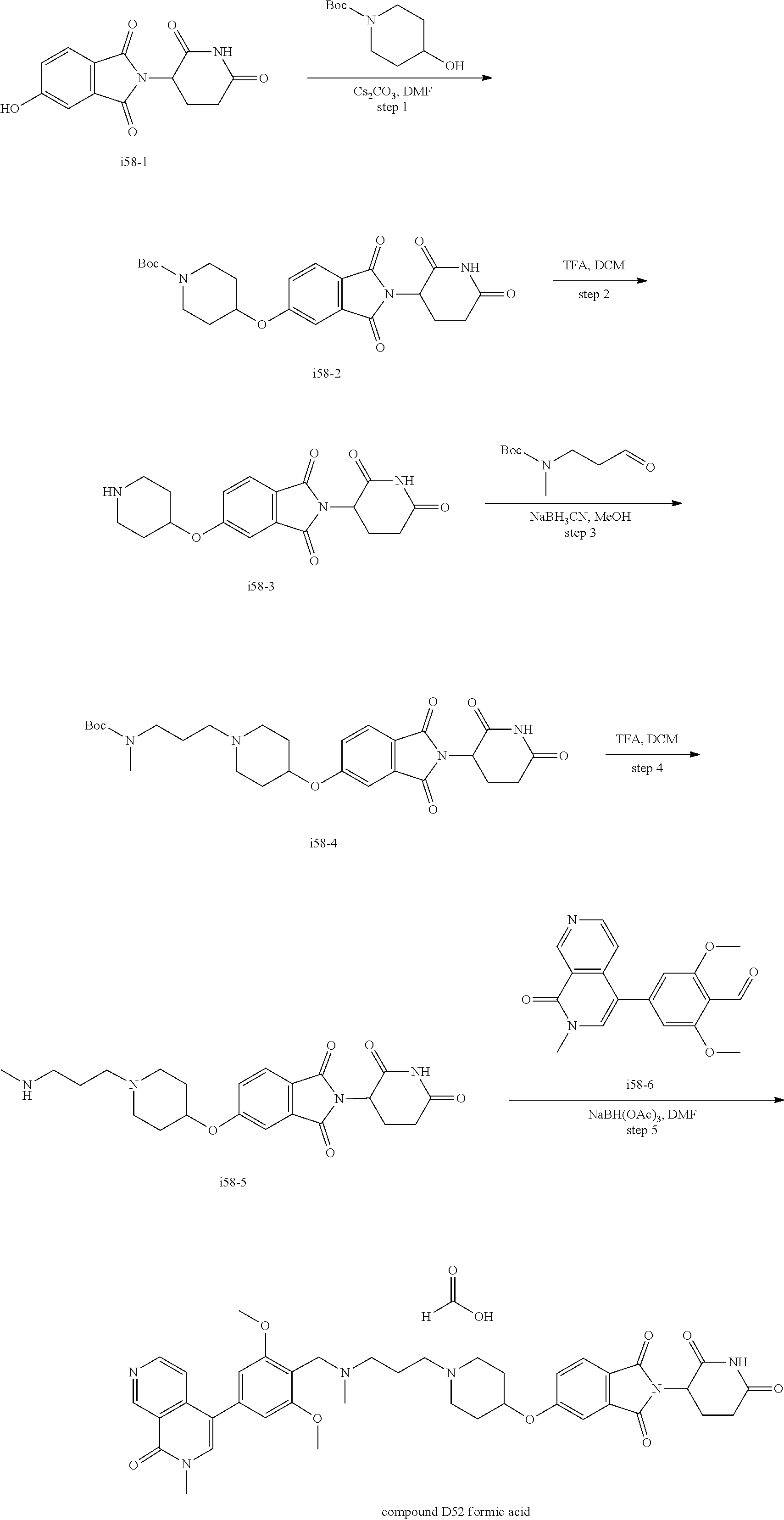

### Step 1: Preparation of tert-butyl 4-((2-(2,6-dioxopiperidin-3-yl)-1,3-dioxoisoindolin-5-yl)oxy)piperidine-1-carboxylate (i58-2)

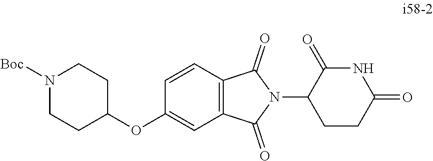 [SMILES: CC(C)(C)OC(=O)N1CCC(Oc2ccc3c(c2)C(=O)N(C2CCC(=O)NC2=O)C3=O)CC1]

A mixture of 2-(2,6-dioxopiperidin-3-yl)-5-hydroxyisoindole-1,3-dione (1.00 g, 3.647 mmol, 1.00 equiv), tert-butyl 4-bromopiperidine-1-carboxylate (0.96 g, 3.634 mmol, 1.00 equiv) and Cs₂CO₃ (2.38 g, 7.293 mmol, 2.00 equiv) in DMF (20.00 mL) was stirred for overnight at 90° C. under air atmosphere. The resulting mixture was filtered, and the filter cake was washed with EtOAc (3×10 mL). The filtrate was concentrated under reduced pressure. The residue was purified by Prep-TLC (hexane/EtOAc 1:1) to afford tert-butyl4-[[2-(2,6-dioxopiperidin-3-yl)-1,3-dioxoisoindol-5-yl]oxy]piperidine-1-carboxylate (280 mg, 11.19%) as a yellow oil. LCMS (ESI) m/z: [M+H]+=458.19.

### Step 2: Preparation of 2-(2,6-dioxopiperidin-3-yl)-5-(piperidin-4-yloxy)isoindoline-1,3-dione (i58-3)

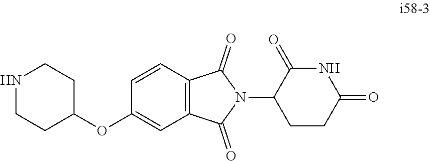 [SMILES: O=C1CCC(N2C(=O)c3ccc(OC4CCNCC4)cc3C2=O)C(=O)N1]

A solution of TFA (1.00 mL) and tert-butyl 4-[[2-(2,6-dioxopiperidin-3-yl)-1,3-dioxoisoindol-5-yl]oxy]piperidine-1-carboxylate(200.00 mg, 0.437 mmol, 1.00 equiv) in DCM (4.00 mL) was stirred for 2 hours at room temperature under air atmosphere. The resulting mixture was concentrated under reduced pressure to afford 2-(2,6-dioxopiperidin-3-yl)-5-(piperidin-4-yloxy) isoindole-1,3-dione (120 mg, 76.81%) as a brown solid. LCMS (ESI) m/z: [M+H]+=358.14.

### Step 3: Preparation of tert-butyl(3-(4-((2-(2,6-dioxopiperidin-3-yl)-1,3-dioxoisoindolin-5-yl)oxy)piperidin-1-yl)propyl)(methyl) carbamate (i58-4)

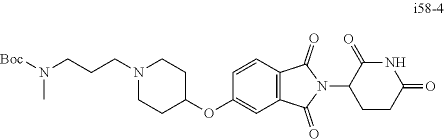 [SMILES: CN(CCCN1CCC(Oc2ccc3c(c2)C(=O)N(C2CCC(=O)NC2=O)C3=O)CC1)C(=O)OC(C)(C)C]

To a stirred solution of 2-(2,6-dioxopiperidin-3-yl)-5-(piperidin-4-yloxy)isoindole-1,3-dione (120.00 mg, 0.336 mmol, 1.00 equiv) and tert-butyl N-methyl-N-(3-oxopropyl)carbamate (62.87 mg, 0.336 mmol, 1.00 equiv) in MeOH (1.50 mL) was added NaBH₃CN (42.20 mg, 0.672 mmol, 2.00 equiv) in portions at room temperature under nitrogen atmosphere. The resulting mixture was stirred for 2 hours at room temperature under nitrogen atmosphere. The residue was purified by Prep-TLC (CH₂Cl₂/MeOH 10:1) to afford tert-butyl N-[3-(4-[[2-(2,6-dioxopiperidin-3-yl)-1,3-dioxoisoindol-5-yl]oxy]piperidin-1-yl)propyl]-N-methylcarbamate (88.00 mg, 49.57%) as a yellow oil. LCMS (ESI) m/z: [M+H]⁺=529.26.

### Step 4: Preparation of 2-(2,6-dioxopiperidin-3-yl)-5-((1-(3-(methylamino)propyl)piperidin-4-yl)oxy)isoindoline-1,3-dione (i58-5)

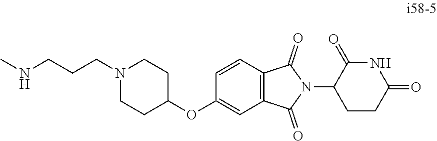 [SMILES: CNCCCN1CCC(Oc2ccc3c(c2)C(=O)N(C2CCC(=O)NC2=O)C3=O)CC1]

A solution of tert-butyl N-[3-(4-[[2-(2,6-dioxopiperidin-3-yl)-1,3-dioxoisoindol-5-yl]oxy]piperidin-1-yl)propyl]-N-methyl carbamate (88.00 mg, 0.166 mmol, 1.00 equiv) and TFA (1.00 mL) in DCM (4.00 mL) was stirred for 1 hour at room temperature. The resulting mixture was concentrated under reduced pressure to afford 2-(2,6-dioxopiperidin-3-yl)-5-((1-(3-(methylamino) propyl)piperidin-4-yl)oxy)isoindoline-1,3-dione (70 mg, 98.50%) as a yellow solid. LCMS (ESI) m/z: [M+H]+=429.21.

### Step 5: Preparation of 5-((1-(3-((2,6-dimethoxy-4-(2-methyl-1-oxo-1,2-dihydro-2,7-naphthyridin-4-yl)benz yl)(methyl)amino)propyl)piperidin-4-yl)oxy)-2-(2,6-dioxopiperidin-3-yl)isoindoline-1,3-dione formic acid (compound D52 formic acid)

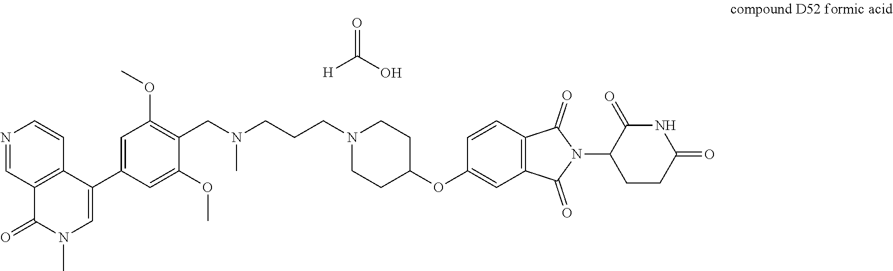 [SMILES: COc1cc(-c2cn(C)c(=O)c3cnccc23)cc(OC)c1CN(C)CCCN1CCC(Oc2ccc3c(c2)C(=O)N(C2CCC(=O)NC2=O)C3=O)CC1 ; O=CO]

A solution of 2-(2,6-dioxopiperidin-3-yl)-5-([1-[3-(methylamino)propyl]piperidin-4-yl]oxy)isoindole-1,3-dione (70.00 mg, 0.163 mmol, 1.00 equiv) and 2,6-dimethoxy-4-(2-methyl-1-oxo-2,7-naphthyridin-4-yl)benzaldehyde (52.99 mg, 0.163 mmol, 1.00 equiv) in DMF (3.00 mL) was stirred for 30 minutes at room temperature. To the above mixture was added NaBH(AcO)₃ (69.25 mg, 0.327 mmol, 2.00 equiv) in portions at room temperature. The resulting mixture was stirred for additional 2 days at 50° C. The mixture was allowed to cool down to room temperature. The residue was purified by reverse flash chromatography (conditions: column, C18 silica gel; mobile phase, MeOH in water, 10% to 50% gradient in 10 minutes; detector, UV 254 nm). The crude product (75 mg) was purified by Prep-HPLC (conditions: SunFire C18 OBD Prep Column, 19 mm×250 mm; mobile phase, Water (0.1% FA) and ACN (hold 7% Phase B in 0 min, up to 12% in 10 minutes); Detector, UV 254/220 nm) to afford 5-([1-[3-([[2,6-dimethoxy-4-(2-methyl-1-oxo-2,7-naphthyridin-4-yl)phenyl]methyl](methyl)amino)propyl]piperidin-4-yl]oxy)-2-(2,6-dioxopiperidin-3-yl)isoindole-1,3-dione (7.8 mg, 6.48%) as a white solid. ¹H NMR (400 MHz, Methanol-d4) δ 9.52 (s, 1H), 8.68 (d, J=5.7 Hz, 1H), 8.42 (brs, 1.4H, FA), 7.83-7.74 (m, 2H), 7.63 (d, J=5.6 Hz, 1H), 7.41 (d, J=2.1 Hz, 1H), 7.36-7.28 (m, 1H), 6.91 (s, 2H), 5.12 (dd, J=12.5, 5.4 Hz, 1H), 4.76 (s, 1H), 4.45 (s, 2H), 4.01 (s, 6H), 3.70 (s, 3H), 3.37 (s, 2H), 3.00 (s, 2H), 2.95-2.84 (m, 4H), 2.82-2.63 (m, 6H), 2.22-2.07 (m, 5H), 1.88 (s, 2H). LCMS (ESI) m/z: [M+H]+=737.40.

### Example 59—Preparation of 5-[3-(4-[[2,6-dimethoxy-4-(2-methyl-1-oxo-2,7-naphthyridin-4-yl)phenyl]methyl]piperazin-1-yl) propoxy]-2-(2,6-dioxopiperidin-3-yl)isoindole-1,3-dione (compound D53)

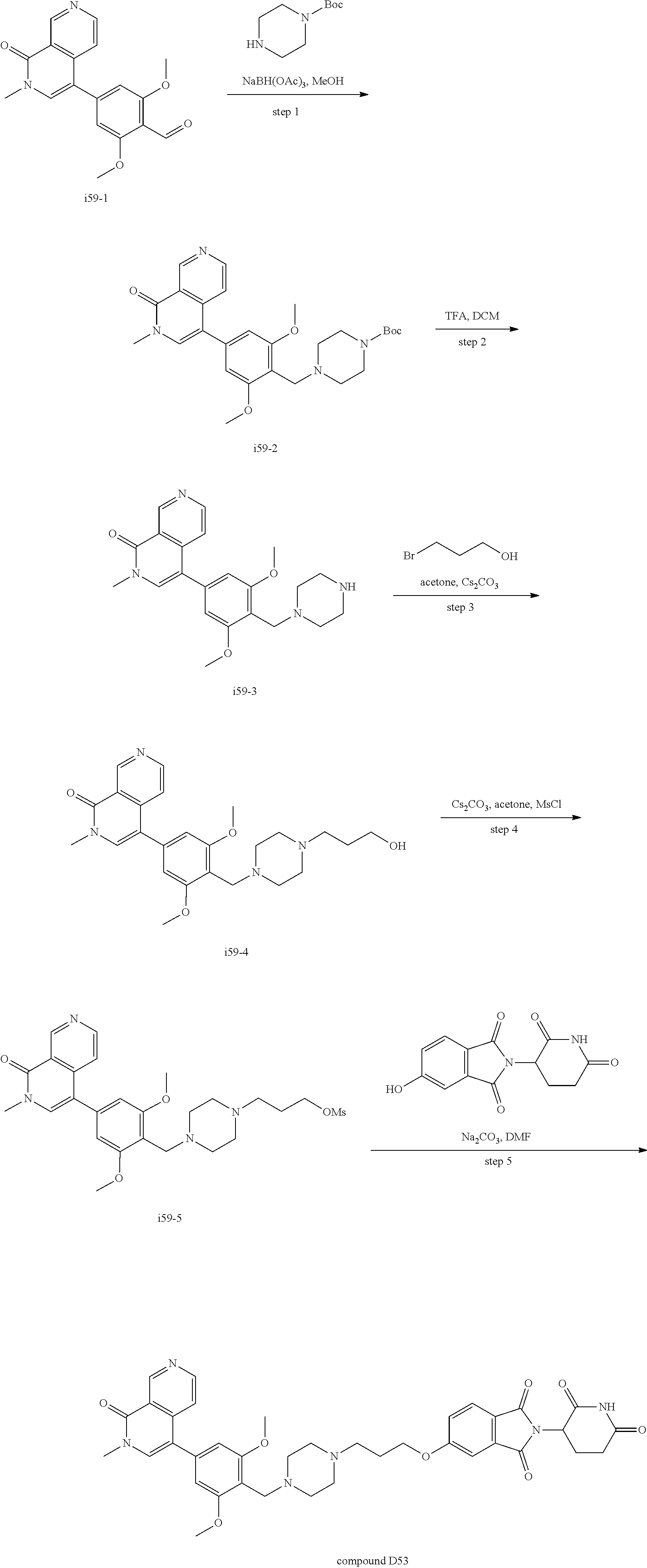

### Step 1: Preparation of tert-butyl 4-[[2,6-dimethoxy-4-(2-methyl-1-oxo-2,7-naphthyridin-4-yl)phenyl]methyl]piperazine-1-carboxylate (i59-2)

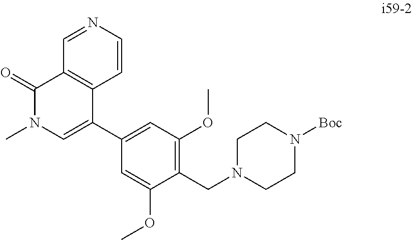 [SMILES: COc1cc(-c2cn(C)c(=O)c3cnccc23)cc(OC)c1CN1CCN(C(=O)OC(C)(C)C)CC1]

To a stirred solution of 2, 6-dimethoxy-4-(2-methyl-1-oxo-2, 7-naphthyridin-4-yl) benzaldehyde (200.00 mg, 0.617 mmol, 1.00 equiv) and tert-butyl piperazine-1-carboxylate (173.00 mg, 0.929 mmol, 1.51 equiv) in MeOH was added NaBH(OAc)₃ (527.00 mg, 2.487 mmol, 4.03 equiv) in portions at room temperature. The resulting mixture was stirred for 3 hours at room temperature. The residue was purified by silica gel column chromatography, eluted with CH₂Cl₂/MeOH (10:1) to afford tert-butyl 4-[[2,6-dimethoxy-4-(2-methyl-1-oxo-2,7-naphthyridin-4-yl)phenyl]methyl]piperazine-1-carboxylate (204 mg, 66.89%) as a light yellow oil. LCMS (ESI) m/z: [M+H]+=495.

### Step 2: Preparation of 4-(3,5-dimethoxy-4-(piperazin-1-ylmethyl) phenyl)-2-methyl-2,7-naphthyridin-1(2H)-one (i59-3)

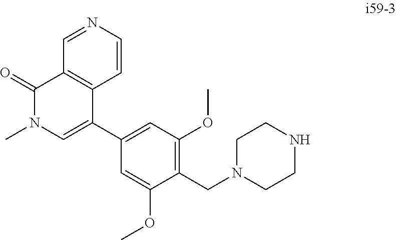 [SMILES: COc1cc(-c2cn(C)c(=O)c3cnccc23)cc(OC)c1CN1CCNCC1]

To a stirred solution of tert-butyl 4-[[2, 6-dimethoxy-4-(2-methyl-1-oxo-2, 7-naphthyridin-4-yl) phenyl]methyl]piperazine-1-carboxylate (204.00 mg, 0.412 mmol, 1.00 equiv) in DCM was added TFA (1.00 mL) dropwise at room temperature. The resulting mixture was stirred for 1 hour at room temperature. The resulting mixture was concentrated under vacuum. The 4-(3,5-dimethoxy-4-(piperazin-1-ylmethyl) phenyl)-2-methyl-2,7-naphthyridin-1(2H)-one (210 mg crude) was used in the next step directly without further purification. LCMS (ESI) m/z: [M+H]+=395.

### Step 3: Preparation of 4-(4-((4-(3-hydroxypropyl) piperazin-1-yl) methyl)-3,5-dimethoxyphenyl)-2-methyl-2,7-naphthyridin-1(2H)-one (i59-4)

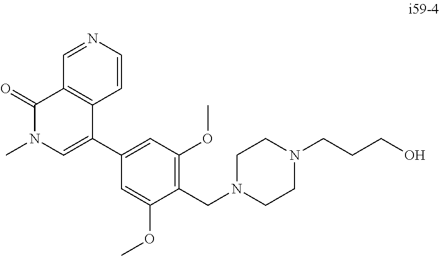 [SMILES: COc1cc(-c2cn(C)c(=O)c3cnccc23)cc(OC)c1CN1CCN(CCCO)CC1]

To a stirred solution of 4-[3,5-dimethoxy-4-(piperazin-1-ylmethyl)phenyl]-2-methyl-2,7-naphthyridin-1-one (200.00 mg, 0.507 mmol, 1.00 equiv) and 3-bromopropanol (140.94 mg, 1.014 mmol, 2.00 equiv) in acetone was added Cs₂CO₃ (330.38 mg, 1.014 mmol, 2.00 equiv) in portions at room temperature. The resulting mixture was stirred for overnight at room temperature. Desired product could be detected by LCMS. The resulting mixture was used in the next step directly without further purification. LCMS (ESI) m/z: [M+H]+=453.

### Step 4: Preparation of 3-(4-[[2,6-dimethoxy-4-(2-methyl-1-oxo-2,7-naphthyridin-4-yl) phenyl]methyl]pipera-zin-1-yl)propyl methanesulfonate (i59-5)

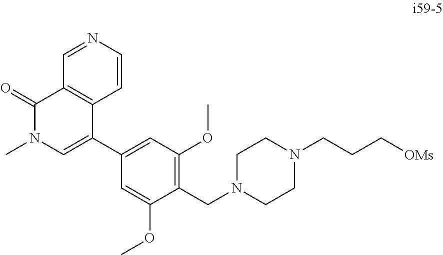 [SMILES: COc1cc(-c2cn(C)c(=O)c3cnccc23)cc(OC)c1CN1CCN(CCCOS(C)(=O)=O)CC1]

To a stirred solution of 4-(4-[[4-(3-hydroxypropyl) piperazin-1-yl]methyl]-3,5-dimethoxyphenyl)-2-methyl-2, 7-naphthyridin-1-one (200.00 mg, 0.442 mmol, 1.00 equiv) and Cs₂CO₃ (287.98 mg, 0.884 mmol, 2.00 equiv) in acetone was added MsCl (101.25 mg, 0.884 mmol, 2.00 equiv) in portions at room temperature. The resulting mixture was stirred for overnight at room temperature. The resulting mixture was concentrated under vacuum. The residue was purified by silica gel column chromatography, eluted with CH₂Cl₂/MeOH (10:1) to afford 3-(4-[[2, 6-dimethoxy-4-(2-methyl-1-oxo-2,7-naphthyridin-4-yl)phenyl]methyl]piperazin-1-yl)propyl methanesulfonate (92 mg, 39.23%) as a light yellow oil. LCMS (ESI) m/z: [M+H]+=531.

### Step 5: Preparation of 5-[3-(4-[[2,6-dimethoxy-4-(2-methyl-1-oxo-2,7-naphthyridin-4-yl)phenyl]methyl]piper-azin-1-yl) propoxy]-2-(2,6-dioxopiperidin-3-yl)isoindole-1,3-dione (compound D53)

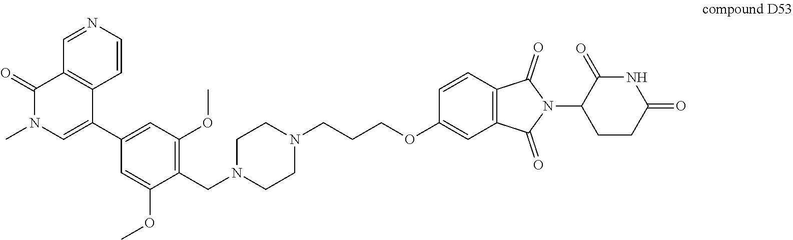 [SMILES: COc1cc(-c2cn(C)c(=O)c3cnccc23)cc(OC)c1CN1CCN(CCCOc2ccc3c(c2)C(=O)N(C2CCC(=O)NC2=O)C3=O)CC1]

To a stirred solution of 3-(4-[[2,6-dimethoxy-4-(2-methyl-1-oxo-2,7-naphthyridin-4-yl)phenyl]methyl]piperazin-1-yl)propyl methanesulfonate (90.00 mg, 0.170 mmol, 1.00 equiv) and 2-(2,6-dioxopiperidin-3-yl)-5-hydroxyisoindole-1,3-dione (47.00 mg, 0.171 mmol, 1.01 equiv) in DMF was added Na₂CO₃ (36.00 mg, 0.340 mmol, 2.00 equiv) in portions at room temperature. The resulting mixture was stirred for 2 hours at 80° C. The crude product was purified by Prep-HPLC (conditions: Xselect CSH F-Phenyl OBD column, 19*250, 5 μm; mobile phase, Water (0.05% TFA) and ACN (hold 5% Phase B in 2 min, up to 22% in 13 minutes); Detector, UV). This resulted in 5-[3-(4-[[2,6-dimethoxy-4-(2-methyl-1-oxo-2,7-naphthyridin-4-yl)phenyl]methyl]piperazin-1-yl)propoxy]-2-(2,6-dioxopiperidin-3-yl)isoindole-1,3-dione (28.1 mg, 23.38%) as an off-white solid. ¹H NMR (300 MHz, Methanol-d4) δ 9.59 (s, 1H), 8.70 (d, J=6.0 Hz, 1H), 7.97 (s, 1H), 7.84 (t, J=7.6 Hz, 2H), 7.45 (d, J=2.1 Hz, 1H), 7.35 (dd, J=8.3, 2.2 Hz, 1H), 6.89 (s, 2H), 5.12 (dd, J=12.4, 5.4 Hz, 1H), 4.49 (s, 2H), 4.30 (t, J=5.7 Hz, 2H), 3.97 (s, 6H), 3.75 (s, 3H), 3.57 (s, 4H), 3.16 (s, 2H), 3.45-3.34 (m, 4H), 2.99-2.65 (m, 3H), 2.25 (s, 2H), 2.19-2.09 (m, 1H). LCMS (ESI) m/z: [M+H]+=709.35.

### Example 60—Preparation of 2-(2,6-dioxopiperidin-3-yl)-4-[4-(9-[[4-(7-hydroxy-2-methyl-1-oxoisoquinolin-4-yl)-2,6-dimethoxyphenyl]methyl]-1-oxa-4,9-diazaspiro[5.5]undecan-4-yl)-4-oxobutoxy]isoindole-1,3-dione formic acid (compound D54 formic acid)

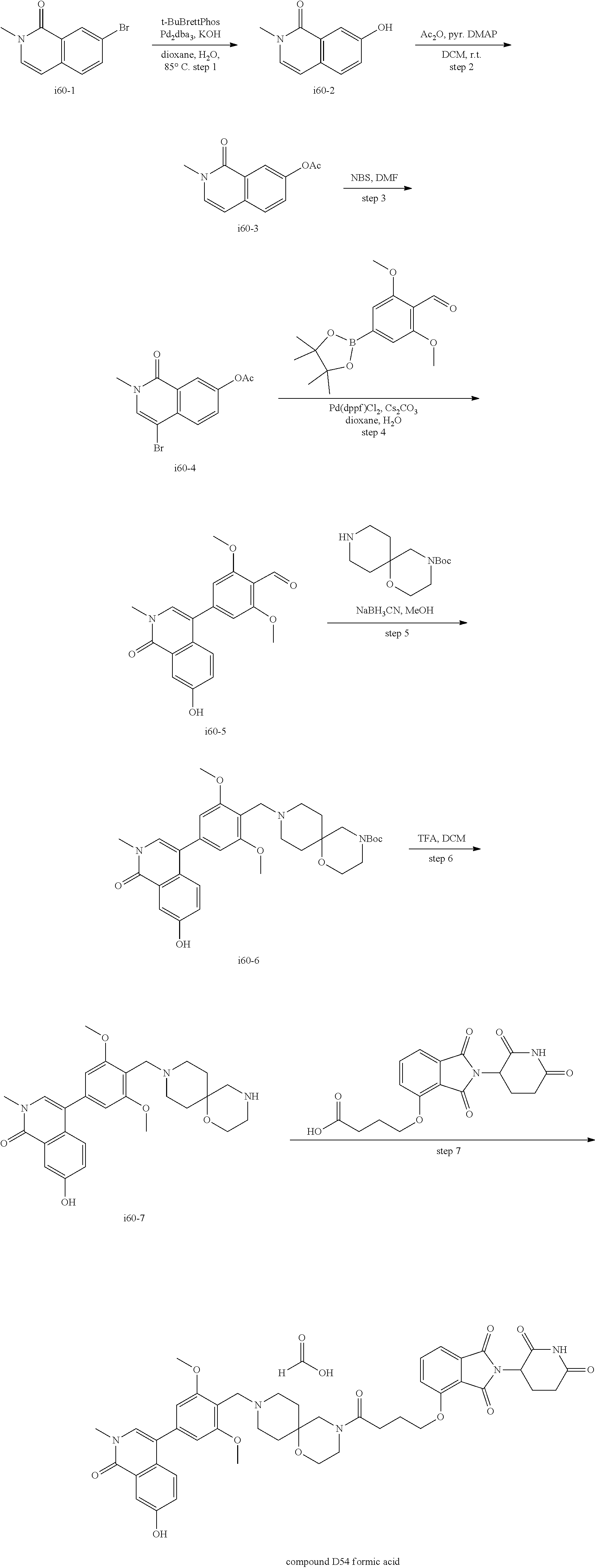

### Step 1: Preparation of 7-hydroxy-2-methylisoquinolin-1-one (i60-2)

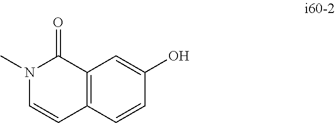

To a mixture of 7-bromo-2-methylisoquinolin-1-one (500 mg, 2.100 mmol, 1.00 equiv), Pd₂(dba)₃ (96.2 mg, 0.105 mmol, 0.05 equiv), tert-BuBrettPhos (101.8 mg, 0.210 mmol, 0.10 equiv), and KOH (353.5 mg, 6.300 mmol, 3.00 equiv) was added dioxane (15 mL) and water (5 mL) at room temperature under nitrogen atmosphere. The resulting mixture was stirred overnight at 85° C. The mixture was acidified pH 4 with 1 M HCl (aq.) and extracted with EtOAc (3×30 mL). The combined organic layers were washed with brine (50 mL) and dried over anhydrous Na₂SO₄. After filtration, the filtrate was concentrated under reduced pressure. The residue was purified by silica gel column chromatography, eluted with PE/EtOAc (1:1 to 3:1) to afford 7-hydroxy-2-methylisoquinolin-1-one (312 mg, 85%) as a grey solid. LCMS (ESI) m/z: [M+H]+=176.

### Step 2: Preparation of 2-methyl-1-oxoisoquinolin-7-yl acetate (i60-3)

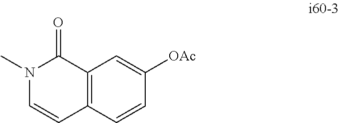 [SMILES: CC(=O)Oc1ccc2ccn(C)c(=O)c2c1]

To a stirred solution/mixture of 7-hydroxy-2-methylisoquinolin-1-one (272 mg, 1.553 mmol, 1.00 equiv) and pyridine (614 mg, 7.763 mmol, 5.00 equiv) in DCM (6 mL) was added DMAP (10 mg, 0.082 mmol, 0.05 equiv) and Ac₂O (46.6 mg, 0.457 mmol, 2.00 equiv) at room temperature. The resulting mixture was stirred for 1 hour at room temperature. The resulting mixture was diluted with water (10 mL) and extracted with DCM (2×20 mL). The combined organic layers were washed with brine, dried over anhydrous Na₂SO₄. After filtration, the filtrate was concentrated under reduced pressure to afford 2-methyl-1-oxoisoquinolin-7-yl acetate (335 mg, 99%) as a light brown solid. LCMS (ESI) m/z: [M+H]+=218.

### Step 3: Preparation of 4-bromo-2-methyl-1-oxoisoquinolin-7-yl acetate (i60-4)

To a stirred solution/mixture of 2-methyl-1-oxoisoquinolin-7-yl acetate (325 mg, 1.496 mmol, 1.00 equiv) in ACN (10 mL) was added NBS (292.9 mg, 1.646 mmol, 1.10 equiv) at room temperature. The resulting mixture was stirred for 0.5 h at room temperature. The resulting mixture was diluted with DCM (30 mL) and washed with 10 mL of water and 10 mL of brine. The organic layer was dried over anhydrous Na₂SO₄. After filtration, the filtrate was concentrated under reduced pressure. The residue was suspended in EtOAc (3 mL), then filtered and the light grey solid was collected as 4-bromo-2-methyl-1-oxoisoquinolin-7-yl acetate (297 mg, 67%). LCMS (ESI) m/z: [M+H]+=296.

### Step 4: Preparation of 4-(7-hydroxy-2-methyl-1-oxoisoquinolin-4-yl)-2,6-dimethoxybenzaldehyde (i60-5)

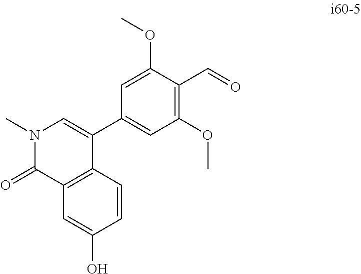 [SMILES: COc1cc(-c2cn(C)c(=O)c3cc(O)ccc23)cc(OC)c1C=O]

To a mixture of 4-bromo-2-methyl-1-oxoisoquinolin-7-yl acetate (217 mg, 0.733 mmol, 1.00 equiv), 2,6-dimethoxy-4-(4,4,5,5-tetramethyl-1,3,2-dioxaborolan-2-yl)benzaldehyde (321.1 mg, 1.099 mmol, 1.50 equiv), Pd(dppf)Cl₂·CH₂Cl₂ (59.8 mg, 0.073 mmol, 0.10 equiv), and Cs₂CO₃ (716.3 mg, 2.198 mmol, 3.00 equiv) was added dioxane (4 mL) and water (1 mL) at room temperature under N₂ atmosphere. The resulting mixture was stirred overnight at 80° C. The resulting mixture was diluted with sat. NH₄Cl solution (10 mL) and extracted with DCM/i-PrOH (3/1) (5×20 mL). The combined organic layers were dried over anhydrous Na₂SO₄. After filtration, the filtrate was concentrated under reduced pressure. The residue was purified by silica gel column chromatography, eluted with DCM/MeOH (100:1 to 20:1) to afford 4-(7-hydroxy-2-methyl-1-oxoisoquinolin-4-yl)-2,6-dimethoxybenzaldehyde (248 mg, quant.) as a light brown solid. LCMS (ESI) m/z: [M+H]⁺=340.

### Step 5: Preparation of tert-butyl 9-[[4-(7-hydroxy-2-methyl-1-oxoisoquinolin-4-yl)-2,6-dimethoxyphenyl]methyl]-1-oxa-4,9-diazaspiro[5.5]undecane-4-carboxylate (i60-6)

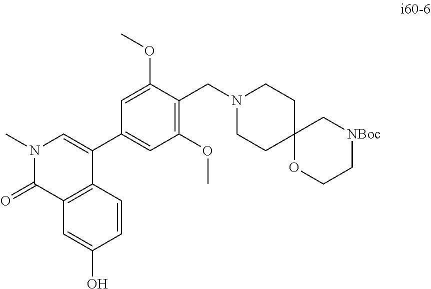 [SMILES: COc1cc(-c2cn(C)c(=O)c3cc(O)ccc23)cc(OC)c1CN1CCC2(CC1)CN(C(=O)OC(C)(C)C)CCO2]

A solution of 4-(7-hydroxy-2-methyl-1-oxoisoquinolin-4-yl)-2,6-dimethoxybenzaldehyde(100 mg, 0.295 mmol, 1.00 equiv) and tert-butyl 1-oxa-4,9-diazaspiro[5.5]undecane-4-carboxylate (83.1 mg, 0.324 mmol, 1.1 equiv) in MeOH (1.5 mL) was stirred for 30 minutes at room temperature. Then NaBH₃CN (125 mg, 1.989 mmol, 6.75 equiv) was added. The resulting mixture was stirred for 2 hours at room temperature. The reaction solution was purified by Prep-TLC (DCM/MeOH 20:1) to afford tert-butyl 9-[[4-(7-hydroxy-2-methyl-1-oxoisoquinolin-4-yl)-2,6-dimethoxyphenyl]methyl]-1-oxa-4,9-diazaspiro[5.5]undecane-4-carboxylate (134 mg, 78%) as a light brown foam. LCMS (ESI) m/z: [M+H]+=580.

### Step 6: Preparation of 4-(3,5-dimethoxy-4-[1-oxa-4,9-diazaspiro[5.5]undecan-9-ylmethyl]phenyl)-7-hydroxy-2-methylisoquinolin-1-one (i60-7)

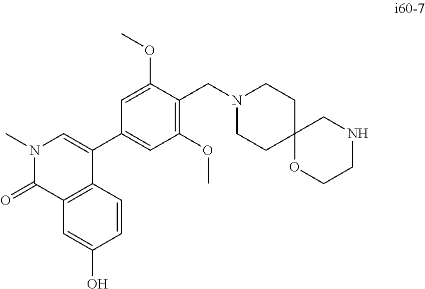 [SMILES: COc1cc(-c2cn(C)c(=O)c3cc(O)ccc23)cc(OC)c1CN1CCC2(CC1)CNCCO2]

To a stirred solution/mixture of tert-butyl 9-[[4-(7-hydroxy-2-methyl-1-oxoisoquinolin-4-yl)-2,6-dimethoxy phenyl]methyl]-1-oxa-4,9-diazaspiro[5.5]undecane-4-carboxylate(134 mg, 0.231 mmol, 1.00 equiv) in DCM (3 mL) was added TFA (1 mL) at room temperature. The resulting mixture was stirred for 30 minutes at room temperature. The mixture was concentrated to dryness to give 4-(3,5-dimethoxy-4-[1-oxa-4,9-diazaspiro[5.5]undecan-9-ylmethyl]phenyl)-7-hydroxy-2-methylisoquinolin-1-one (135 mg, TFA salt, quant.) as a light brown solid. LCMS (ESI) m/z: [M+H]+=480.

### Step 7: Preparation of 2-(2,6-dioxopiperidin-3-yl)-4-[4-(9-[[4-(7-hydroxy-2-methyl-1-oxoisoquinolin-4-yl)-2,6-dimethoxyphenyl]methyl]-1-oxa-4,9-diazaspiro[5.5]undecan-4-yl)-4-oxobutoxy]isoindole-1,3-dione formic acid (compound D54 formic acid)

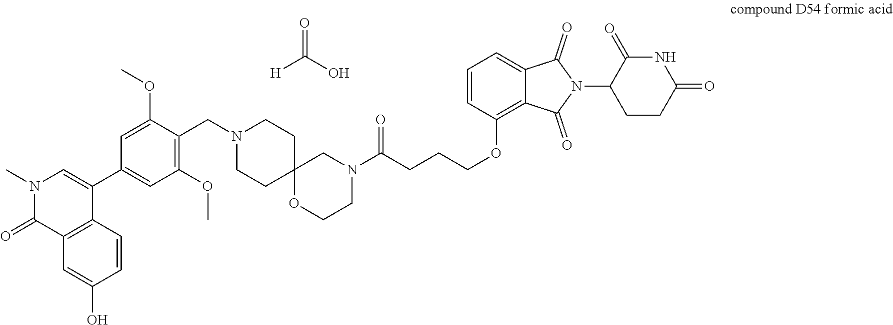 [SMILES: COc1cc(-c2cn(C)c(=O)c3cc(O)ccc23)cc(OC)c1CN1CCC2(CC1)CN(C(=O)CCCOc1cccc3c1C(=O)N(C1CCC(=O)NC1=O)C3=O)CCO2 ; O=CO]

To a stirred solution of 4-[[2-(2,6-dioxopiperidin-3-yl)-1,3-dioxoisoindol-4-yl]oxy]butanoic acid (33.8 mg, 0.094 mmol, 0.90 equiv) in DMF (1 mL) was added EDCl (40.0 mg, 0.209 mmol, 2.00 equiv) and HOBt (28.2 mg, 0.209 mmol, 2.00 equiv) at room temperature. The resulting mixture was stirred at room temperature for 20 minutes followed by addition of 4-(3,5-dimethoxy-4-[1-oxa-4,9-diazaspiro [5.5]undecan-9-ylmethyl]phenyl)-7-hydroxy-2-methylisoquinolin-1-one (50.0 mg, 0.104 mmol, 1.00 equiv) and DIEA (67.4 mg, 0.521 mmol, 5.00 equiv). After stirring for 3 hours at room temperature, the reaction mixture was purified by Prep-HPLC (conditions: SunFire Prep C18 OBD Column, 19×150 mm 5 μm 10 nm; Mobile Phase A: Water (0.1% FA), Mobile Phase B:ACN; Flow rate:25 mL/minute; Gradient:15 B to 24 B in 12 minutes; 254/220 nm; R_{T}:11.28 minutes) to afford 2-(2,6-dioxopiperidin-3-yl)-4-[4-(9-[[4-(7-hydroxy-2-methyl-1-oxoisoquinolin-4-yl)-2,6-dimethoxyphenyl]methyl]-1-oxa-4,9-diazaspiro[5.5]undecan-4-yl)-4-oxobutoxy]isoindole-1,3-dione formic acid (11.5 mg, 13%) as an off-white solid. ¹H NMR (400 MHz, Methanol-d4) δ 8.55 (s, 0.5H, FA), 7.84-7.75 (m, 2H), 7.56 (dd, J=8.8, 3.3 Hz, 1H), 7.47 (dd, J=7.6, 2.7 Hz, 2H), 7.31-7.19 (m, 2H), 6.82 (d, J=8.8 Hz, 2H), 5.12 (dd, J=12.5, 5.6 Hz, 1H), 4.40-4.20 (m, 4H), 3.93 (d, J=12.4 Hz, 6H), 3.78-3.62 (m, 7H), 3.58-3.48 (m, 2H), 3.30-3.17 (m, 4H), 2.97-2.53 (m, 5H), 2.24-1.99 (m, 5H), 1.95-1.71 (s, 2H). LCMS (ESI) m/z: [M+H]+=822.40.

### Example 61—Preparation of 3-[([[2,6-dimethoxy-4-(2-methyl-1-oxo-2,7-naphthyridin-4-yl)phenyl]methyl](methyl)amino)methyl]-N-[2-[2-(2-[[2-(2,6-dioxopiperidin-3-yl)-1,3-dioxoisoindol-4-yl]amino]ethoxy)ethoxy]ethyl]bicyclo[1.1.1]pentane-1-carboxamide (compound D55)

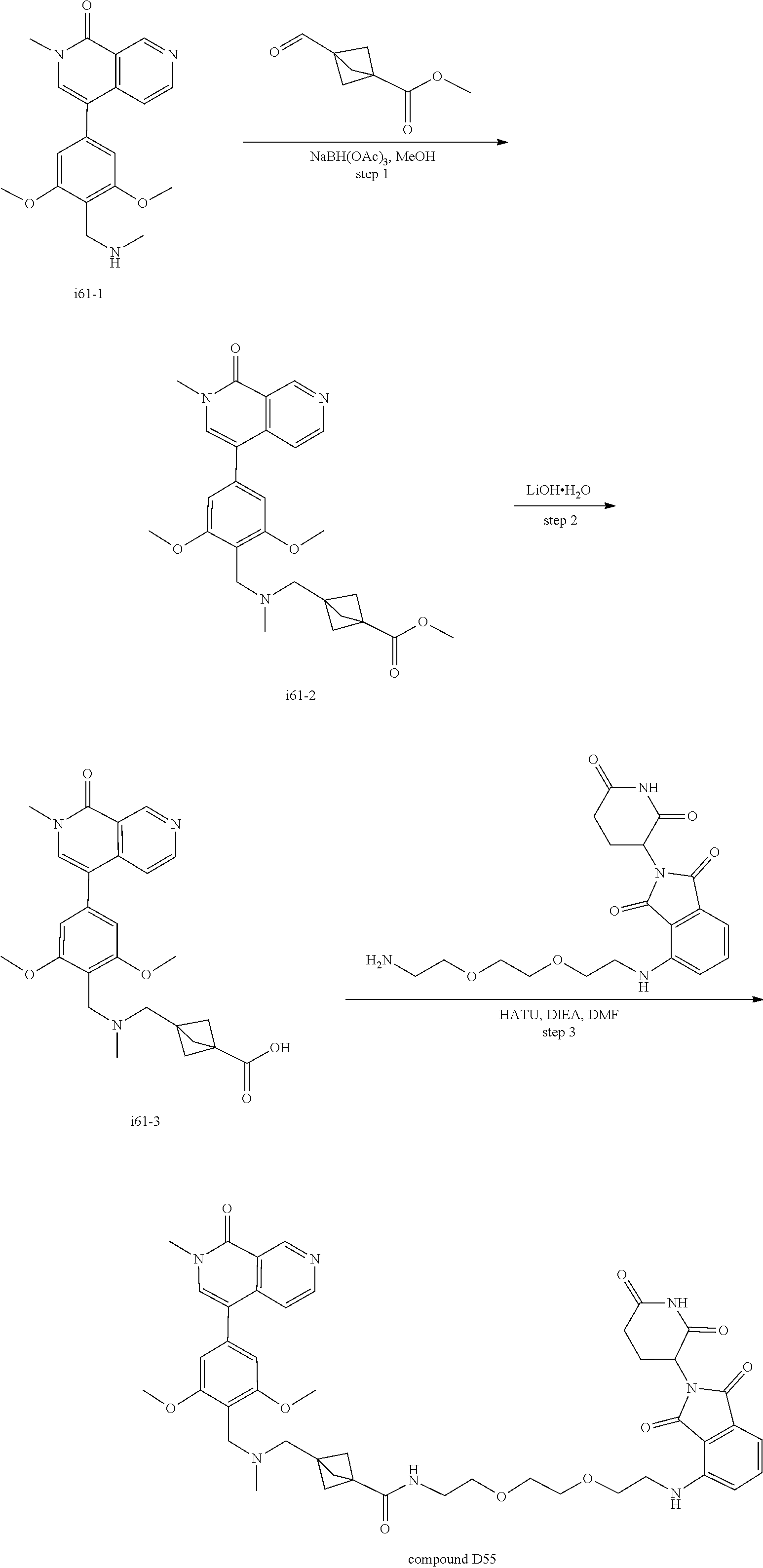

### Step 1: Preparation of methyl 3-[([[2,6-dimethoxy-4-(2-methyl-1-oxo-2,7-naphthyridin-4-yl)phenyl]methyl](methyl)amino)methyl]bicyclo[1.1.1]pentane-1-carboxylate (i61-2)

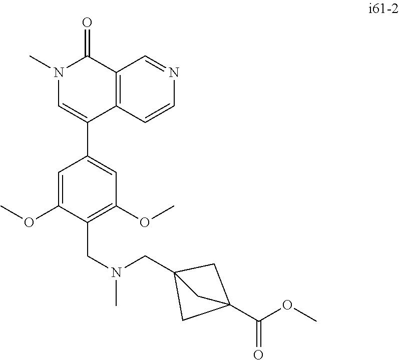

To a stirred solution of 4-[3,5-dimethoxy-4-[(methylamino)methyl]phenyl]-2-methyl-2,7-naphthyridin-1-one (264.00 mg, 0.778 mmol, 1.20 equiv) and methyl 3-formylbicyclo[1.1.1]pentane-1-carboxylate (100.00 mg, 0.649 mmol, 1.00 equiv) in MeOH was added NaBH(OAc)₃ (549.91 mg, 2.595 mmol, 4.00 equiv) in portions at room temperature. The resulting solution was stirred for 4 hours at room temperature. Desired product could be detected by LCMS. The resulting mixture was concentrated under reduced pressure. The residue was purified by silica gel column chromatography, eluted with CH₂Cl₂/MeOH (8:1) to afford methyl 3-[([[2,6-dimethoxy-4-(2-methyl-1-oxo-2,7-naphthyridin-4-yl)phenyl]methyl](methyl)amino)-methyl]bicyclo[1.1.1]pentane-1-carboxylate (220 mg, 71.02%) as a light yellow oil. LCMS (ESI) m/z: [M+H]+=478.

### Step 2: Preparation of 3-(((2,6-dimethoxy-4-(2-methyl-1-oxo-1,2-dihydro-2,7-naphthyridin-4-yl)benzyl) (methyl)amino)methyl)bicyclo[1.1.1]pentane-1-carboxylic acid (i61-3)

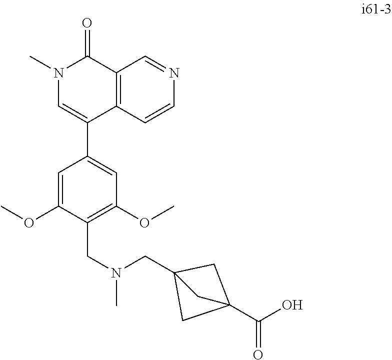 [SMILES: COc1cc(-c2cn(C)c(=O)c3cnccc23)cc(OC)c1CN(C)CC12CC(C(=O)O)(C1)C2]

To a stirred solution of methyl 3-[([[2,6-dimethoxy-4-(2-methyl-1-oxo-2,7-naphthyridin-4-yl)phenyl]methyl](methyl)amino)methyl]bicyclo[1.1.1]pentane-1-carboxylate (200.00 mg, 0.419 mmol, 1.00 equiv) and LiOH·H₂O (35.15 mg, 0.838 mmol, 2.00 equiv) in THF (6 mL) was added H₂O (2.00 mL) dropwise at room temperature. The resulting mixture was stirred for overnight at room temperature. The mixture was acidified to pH<7 with conc. HCl. The resulting mixture was concentrated under vacuum. The 3-(((2,6-dimethoxy-4-(2-methyl-1-oxo-1,2-dihydro-2,7-naphthyridin-4-yl)benzyl)(methyl)amino)methyl)-bicyclo[1. 1.1]pentane-1-carboxylic acid (215 mg crude) was used in the next step directly without further purification. LCMS (ESI) m/z: [M+H]+=464.

### Step 3: Preparation of 3-[([[2,6-dimethoxy-4-(2-methyl-1-oxo-2,7-naphthyridin-4-yl)phenyl]methyl](methyl)-amino)methyl]-N-[2-[2-(2-[[2-(2,6-dioxopiperidin-3-yl)-1,3-dioxoisoindol-4-yl]amino]ethoxy)ethoxy]ethyl]bic-yclo[1.1.1]pentane-1-carboxamide (compound D55)

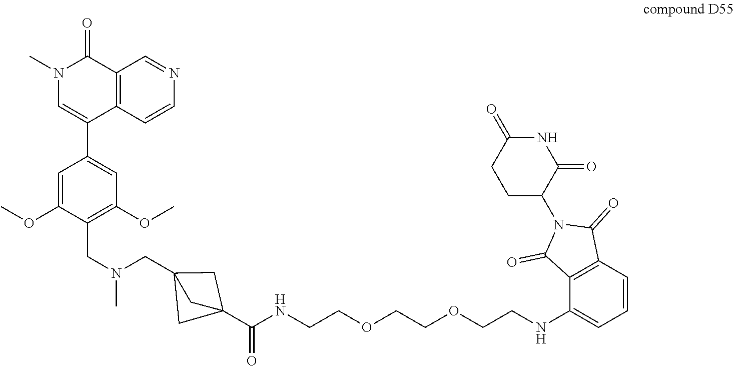 [SMILES: COc1cc(-c2cn(C)c(=O)c3cnccc23)cc(OC)c1CN(C)CC12CC(C(=O)NCCOCCOCCNc3cccc4c3C(=O)N(C3CCC(=O)NC3=O)C4=O)(C1)C2]

To a stirred solution of 3-[([[2,6-dimethoxy-4-(2-methyl-1-oxo-2,7-naphthyridin-4-yl)phenyl]methyl](methyl)amino)methyl]bicyclo[1.1.1]pentane-1-carboxylic acid (50.00 mg, 0.108 mmol, 1.00 equiv) and 4-([2-[2-(2-aminoethoxy)ethoxy]ethyl]amino)-2-(2,6-dioxopiperidin-3-yl)isoindole-1,3-dione (65.44 mg, 0.162 mmol, 1.50 equiv) in DMF were added DIEA (55.76 mg, 0.431 mmol, 4.00 equiv) and HATU (61.52 mg, 0.162 mmol, 1.50 equiv) in portions at room temperature. The resulting mixture was stirred for 3 h at room temperature. The crude product was purified by Prep-HPLC with the following conditions (NB-Prep-HPLC-01): Column, XSelect CSH Prep C18 OBD Column, 5 μm, 19*150 mm; mobile phase, Water (0.05% TFA) and ACN (16% PhaseB up to 17% in 20 min hold 17% in 8 minutes); Detector, uv. This resulted in 3-[([[2,6-dimethoxy-4-(2-methyl-1-oxo-2,7-naphthyridin-4-yl)phenyl]methyl](methyl)ami-no) methyl]-N-[2-[2-(2-[[2-(2,6-dioxopiperidin-3-yl)-1,3-dioxoisoindol-4-yl]amino]ethoxy)ethoxy]ethyl]bicyc-lo [1.1.1]pentane-1-carboxamide; formic acid (4.1 mg, 4.24%) as a yellow solid. ¹H NMR (300 MHz, Methanol-d4) δ 9.55 (s, 1H), 8.70 (d, J=5.7 Hz, 1H), 8.55 (s, 1H), 7.78 (s, 1H), 7.65 (d, J=5.9 Hz, 1H), 7.52 (dd, J=8.6, 7.1 Hz, 1H), 7.09 (d, J=8.5 Hz, 1H), 6.99 (d, J=7.1 Hz, 1H), 6.84 (s, 2H), 5.07 (dd, J=12.4, 5.4 Hz, 1H), 4.22 (s, 2H), 3.95 (s, 6H), 3.77 (t, J=5.2 Hz, 2H), 3.73 (s, 3H), 3.71-3.65 (m, 4H), 3.59 (t, J=5.4 Hz, 2H), 3.52 (t, J=5.2 Hz, 2H), 3.44-3.38 (m, 2H), 3.28-3.24 (m, 1H), 2.91-2.81 (m, 1H), 2.80-2.77 (m, 1H), 2.75-2.69 (m, 1H), 2.66 (s, 3H), 2.20 (s, 6H), 2.17-2.05 (m, 2H). LCMS (ESI) m/z: [M+H]+=850.45.

### Example 62—Preparation of 3-[([[2,6-dimethoxy-4-(2-methyl-1-oxo-2,7-naphthyridin-4-yl)phenyl]methyl](methyl)amino)methyl]-N-(5-[[2-(2,6-dioxopiperidin-3-yl)-1,3-dioxoisoindol-4-yl]amino]pentyl)bicyclo[1.1.1]pentane-1-carboxamide (compound D56)

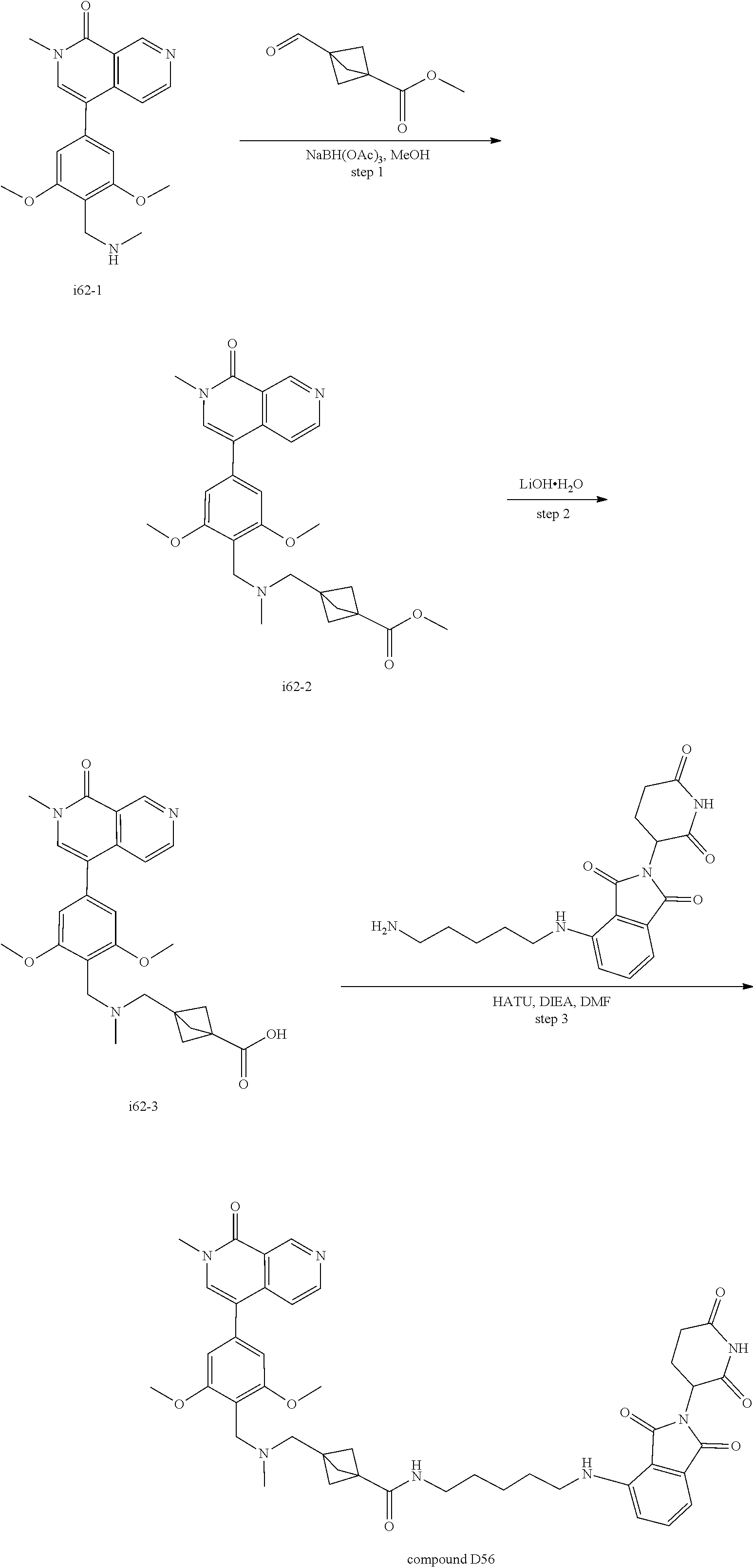

### Step 1: Preparation of methyl 3-[([[2,6-dimethoxy-4-(2-methyl-1-oxo-2,7-naphthyridin-4-yl)phenyl]methyl](methyl)amino)methyl]bicyclo[1.1.1]pentane-1-carboxylate (i62-2)

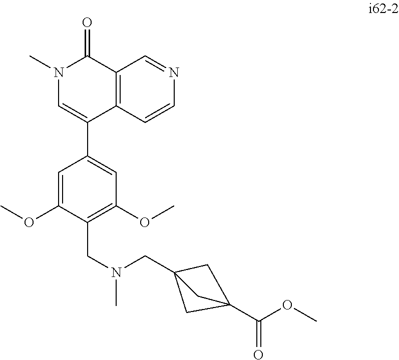

To a stirred solution of 4-[3,5-dimethoxy-4-[(methylamino)methyl]phenyl]-2-methyl-2,7-naphthyridin-1-one (264.18 mg, 0.778 mmol, 1.20 equiv) and methyl 3-formylbicyclo[1.1.1]pentane-1-carboxylate (100.00 mg, 0.649 mmol, 1.00 equiv) in MeOH was added NaBH(OAc)₃ (549.91 mg, 2.595 mmol, 4.00 equiv) in portions at room temperature. The resulting solution was stirred for 4 hours at room temperature. Desired product could be detected by LCMS. The resulting mixture was concentrated under reduced pressure. The residue was purified by silica gel column chromatography, eluted with CH₂Cl₂/MeOH (8:1) to afford methyl 3-[([[2,6-dimethoxy-4-(2-methyl-1-oxo-2,7-naphthyridin-4-yl)phenyl]methyl](methyl)amino)-methyl]bicyclo[1.1.1]pentane-1-carboxylate (220 mg, 71.02%) as a light yellow oil. LCMS (ESI) m/z: [M+H]+=478.

### Step 2: Preparation of 3-(((2,6-dimethoxy-4-(2-methyl-1-oxo-1,2-dihydro-2,7-naphthyridin-4-yl)benzyl) (methyl)amino)methyl)bicyclo[1.1.1]pentane-1-carboxylic acid (i62-3)

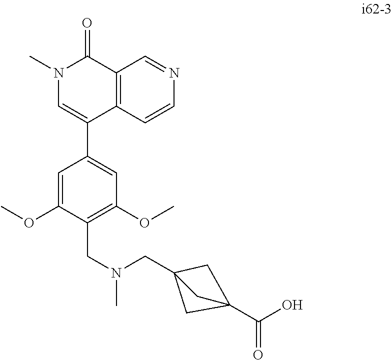 [SMILES: COc1cc(-c2cn(C)c(=O)c3cnccc23)cc(OC)c1CN(C)CC12CC(C(=O)O)(C1)C2]

To a stirred solution of methyl 3-[([[2,6-dimethoxy-4-(2-methyl-1-oxo-2,7-naphthyridin-4-yl)phenyl]methyl](methyl)amino)methyl]bicyclo[1.1.1]pentane-1-carboxylate (200.00 mg, 0.419 mmol, 1.00 equiv) and LiOH·H₂O (35.15 mg, 0.838 mmol, 2.00 equiv) in THF (6 mL) was added H₂O (2.00 mL) dropwise at room temperature. The resulting mixture was stirred for overnight at room temperature. The mixture was acidified to pH<7 with conc. HCl. The resulting mixture was concentrated under vacuum. The 3-(((2,6-dimethoxy-4-(2-methyl-1-oxo-1,2-dihydro-2,7-naphthyridin-4-yl)benzyl)(methyl)amino)methyl)-bicyclo[1. 1.1]pentane-1-carboxylic acid (215 mg crude) was used in the next step directly without further purification. LCMS (ESI) m/z: [M+H]+=464.

### Step 3: Preparation of 3-[([[2,6-dimethoxy-4-(2-methyl-1-oxo-2,7-naphthyridin-4-yl)phenyl]methyl](methyl) amino)methyl]-N-(5-[[2-(2,6-dioxopiperidin-3-yl)-1,3-dioxoisoindol-4-yl]amino]pentyl)bicyclo[1.1.1]pentane-1-carboxamide (compound D56)

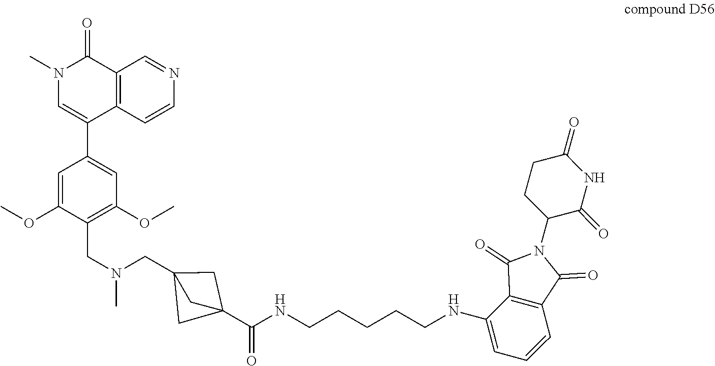 [SMILES: COc1cc(-c2cn(C)c(=O)c3cnccc23)cc(OC)c1CN(C)CC12CC(C(=O)NCCCCCNc3cccc4c3C(=O)N(C3CCC(=O)NC3=O)C4=O)(C1)C2]

To a stirred solution of 3-[([[2,6-dimethoxy-4-(2-methyl-1-oxo-2,7-naphthyridin-4-yl)phenyl]methyl](methyl)amino)methyl]bicyclo[1.1.1]pentane-1-carboxylic acid (50.00 mg, 0.108 mmol, 1.00 equiv) and 4-((5-aminopentyl)amino)-2-(2,6-dioxopiperidin-3-yl)isoindoline-1,3-dione (65.44 mg, 0.162 mmol, 1.50 equiv) in DMF was added DIEA (55.76 mg, 0.431 mmol, 4.00 equiv) and HATU (61.52 mg, 0.162 mmol, 1.50 equiv) in portions at room temperature. The resulting mixture was stirred for 3 hours at room temperature. The crude product was purified by Prep-HPLC (conditions: XSelect CSH Prep C18 OBD Column, 5 μm, 19*150 mm; mobile phase, Water (0.05% TFA) and ACN (16% Phase B up to 17% in 20 min hold 17% in 8 minutes); Detector, UV). This resulted in 3-(((2,6-dimethoxy-4-(2-methyl-1-oxo-1,2-dihydro-2,7-naphthyridin-4-yl)benzyl)(methyl)amino)-methyl)-N-(5-((2-(2,6-dioxopiperidin-3-yl)-1,3-dioxoisoindolin-4-yl)amino)pentyl)bicyclo[1.1.1]pentane-1-carboxamide (12.3 mg, 13.42%) as a yellow solid. LCMS (ESI) m/z: [M+H]+=804.45.

### Example 63—Preparation of 3-([[2,6-dimethoxy-4-(2-methyl-1-oxo-2,7-naphthyridin-4-yl)phenyl]methyl](methyl) amino)-N-(5-[[2-(2,6-dioxopiperidin-3-yl)-1,3-dioxoisoindol-4-yl]amino]pentyl)bicyclo[1.1.1]pentane-1-carboxamide; formic acid (compound D57 formic acid)

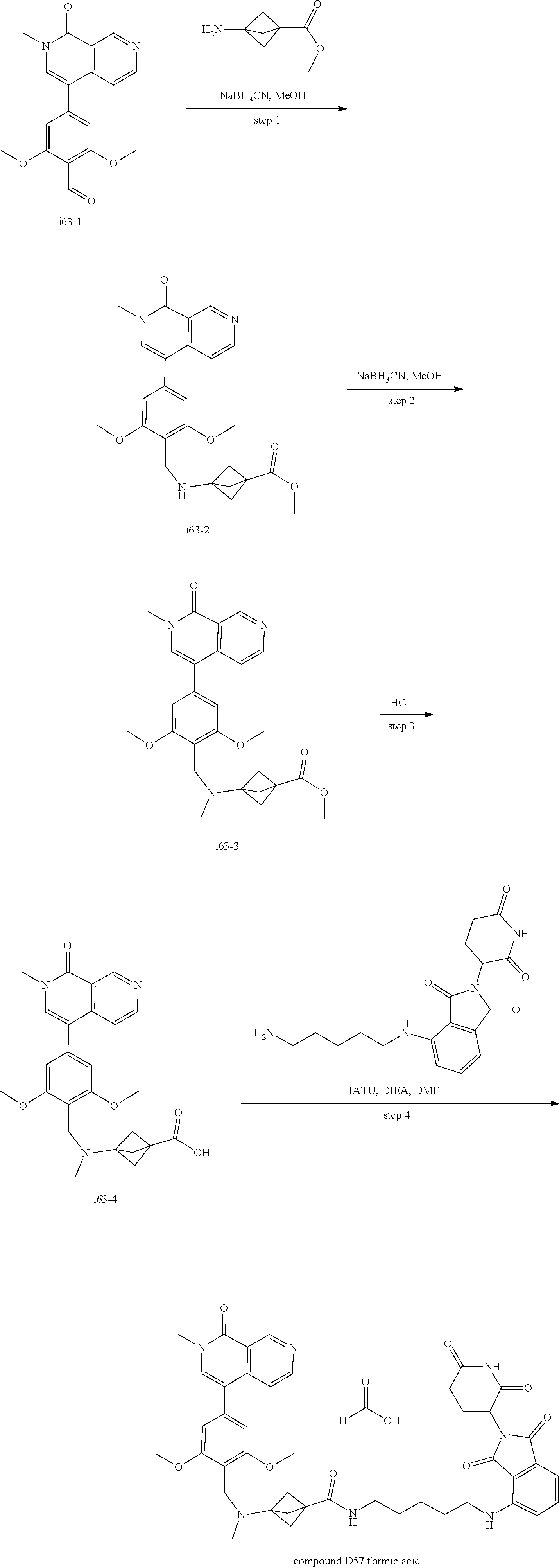

### Step 1: Preparation of methyl-3-((2,6-dimethoxy-4-(2-methyl-1-oxo-1,2-dihydro-2,7-naphthyridin-4-yl)benzyl)amino)bicyclo[1.1.1]pentane-1-carboxylate (i63-2)

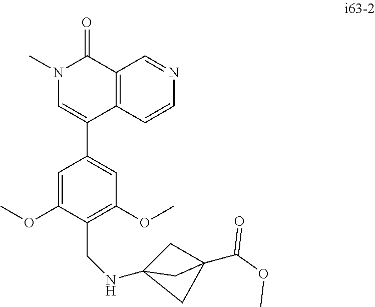 [SMILES: COC(=O)C12CC(NCc3c(OC)cc(-c4cn(C)c(=O)c5cnccc45)cc3OC)(C1)C2]

To a solution of methyl 3-aminobicyclo[1.1.1]pentane-1-carboxylate hydrochloride (195.2 mg, 1.099 mmol, 1.10 equiv) in MeOH (5.00 mL) was added Et₃N (111.0 mg, 1.099 mmol, 1.10 equiv), and then 2,6-dimethoxy-4-(2-methyl-1-oxo-2,7-naphthyridin-4-yl)benzaldehyde (324.0 mg, 0.999 mmol, 1.00 equiv) was added. After 10 minutes stirring, NaBH₃CN (125.6 mg, 1.998 mmol, 2.00 equiv) was added in portions at ambient atmosphere. The resulting mixture was concentrated after stirring for 1 hour at room temperature. The mixture was used in the next step directly without further purification. LCMS (ESI) m/z: [M+H]⁺=450.

### Step 2: Preparation of methyl-3-([[2,6-dimethoxy-4-(2-methyl-1-oxo-2,7-naphthyridin-4-yl)phenyl]methyl](methyl)amino)bicyclo[1.1.1]pentane-1-carboxylate (i63-3)

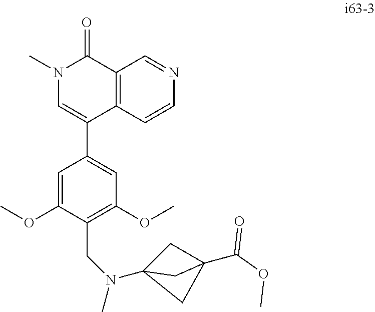 [SMILES: COC(=O)C12CC(N(C)Cc3c(OC)cc(-c4cn(C)c(=O)c5cnccc45)cc3OC)(C1)C2]

To a solution of crude methyl-3-([[2,6-dimethoxy-4-(2-methyl-1-oxo-2,7-naphthyridin-4-yl)phenyl]methyl]amino)bicyclo[1.1.1]pentane-1-carboxylate obtained last step in MeOH (5.00 mL, 12.349 mmol) was added formaldehyde in water (226.0 μL). After 10 min stirring, NaBH₃CN (125.8 mg, 2.002 mmol, 2.00 equiv) was added in portions at ambient atmosphere. The resulting mixture was concentrated after stirring for 1 hour at room temperature. The mixture was purified by Prep-TLC (EtOAc) to afford methyl-3-([[2,6-dimethoxy-4-(2-methyl-1-oxo-2,7-naphthyridin-4-yl)phenyl]methyl](methyl)amino)bicyclo[1.1.1]pentane-1-carboxylate (120 mg, 24.8%) as a light yellow solid. LCMS (ESI) m/z: [M+H]⁺=464.

### Step 3: Preparation of 3-((2,6-dimethoxy-4-(2-methyl-1-oxo-1,2-dihydro-2,7-naphthyridin-4-yl)benzyl) (methyl)amino)bicyclo[1.1.1]pentane-1-carboxylic acid (i63-4)

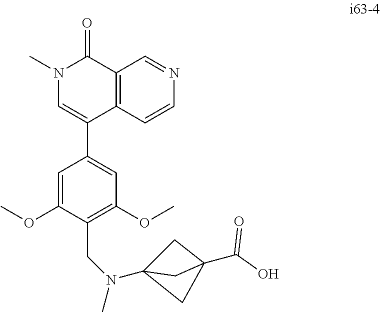 [SMILES: COc1cc(-c2cn(C)c(=O)c3cnccc23)cc(OC)c1CN(C)C12CC(C(=O)O)(C1)C2]

A mixture of methyl 3-([[2,6-dimethoxy-4-(2-methyl-1-oxo-2,7-naphthyridin-4-yl)phenyl]methyl](methyl)amino)bicyclo[1.1.1]pentane-1-carboxylate (120.0 mg, 0.259 mmol, 1.00 equiv) in conc. HCl (2.00 mL) was stirred for 1 hour at 90° C. The resulting mixture was concentrated under vacuum. The crude product was used in the next step directly without further purification. LCMS (ESI) m/z: [M+H]⁺=450.

### Step 4: Preparation of 3-([[2,6-dimethoxy-4-(2-methyl-1-oxo-2,7-naphthyridin-4-yl)phenyl]methyl](methyl)amino)-N-(5-[[2-(2,6-dioxopiperidin-3-yl)-1,3-dioxoisoindol-4-yl]amino]pentyl)bicyclo[1.1.1]pentane-1-carboxamide formic acid (compound D57 formic acid)

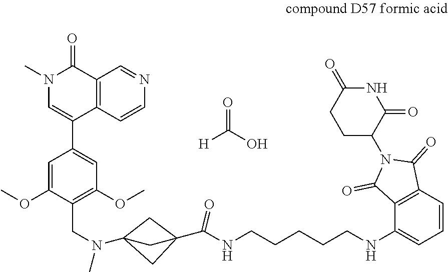 [SMILES: COc1cc(-c2cn(C)c(=O)c3cnccc23)cc(OC)c1CN(C)C12CC(C(=O)NCCCCCNc3cccc4c3C(=O)N(C3CCC(=O)NC3=O)C4=O)(C1)C2 ; O=CO]

To a stirred mixture of 3-([[2,6-dimethoxy-4-(2-methyl-1-oxo-2,7-naphthyridin-4-yl)phenyl]methyl](methyl)amino)bicyclo[1.1.1]pentane-1-carboxylic acid hydrochloride (50 mg, 0.103 mmol, 1.00 equiv) and 4-[(5-aminopentyl)amino]-2-(2,6-dioxopiperidin-3-yl)isoindole-1,3-dione; trifluoroacetic acid (53.5 mg, 0.113 mmol, 1.10 equiv) in DMF (2.00 mL) was added DIEA (39.9 mg, 0.309 mmol, 3.00 equiv). The mixture was stirred at room temperature for 5 minutes, and then HATU (78.2 mg, 0.206 mmol, 2.00 equiv) was added. After stirring at room temperature for 2 hours, the mixture was purified by Prep-HPLC (conditions: X-select CSH F-Phenyl OBD Column 19*150 mm 5 μm, mobile phase, Water (0.05% TFA) and ACN (10% Phase B up to 26% in 15 minutes)). This resulted in of 3-([[2,6-dimethoxy-4-(2-methyl-1-oxo-2,7-naphthyridin-4-yl)phenyl]methyl](methyl)amino)-N-(5-[[2-(2,6-dioxopiperidin-3-yl)-1,3-dioxoisoindol-4-yl]amino]pentyl)bicyclo[1.1.1]pentane-1-carboxamide formic acid (15.2 mg, 17.2%) as a yellow solid. ¹H NMR (300 MHz, Methanol-d4) δ 9.57 (s, 1H), 8.71 (d, J=5.9 Hz, 1H), 8.18 (brs, 0.4H, FA), 7.86 (s, 1H), 7.72 (s, 1H), 7.58 (dd, J=8.6, 7.1 Hz, 1H), 7.07 (dd, J=7.8, 5.2 Hz, 2H), 6.90 (s, 2H), 5.06 (dd, J=12.0, 5.4 Hz, 1H), 4.51 (s, 2H), 3.99 (s, 6H), 3.73 (s, 3H), 3.41-3.35 (m, 2H), 3.31-3.23 (m, 2H), 2.89-2.64 (m, 6H), 2.42 (s, 6H), 2.17-2.08 (m, 1H), 1.78-1.67 (m, 2H), 1.66-1.56 (m, 2H), 1.54-1.43 (m, 2H). LCMS (ESI) m/z: [M+H]⁺=790.40.

### Example 64—Preparation of 3-([[2,6-dimethoxy-4-(2-methyl-1-oxo-2,7-naphthyridin-4-yl)phenyl]methyl](methyl)amino)-N-[2-[2-(2-[[2-(2,6-dioxopiperidin-3-yl)-1,3-dioxoisoindol-4-yl]amino]ethoxy)ethoxy]ethyl]bicyclo[1.1.1]pentane-1-carboxamide (compound D58)

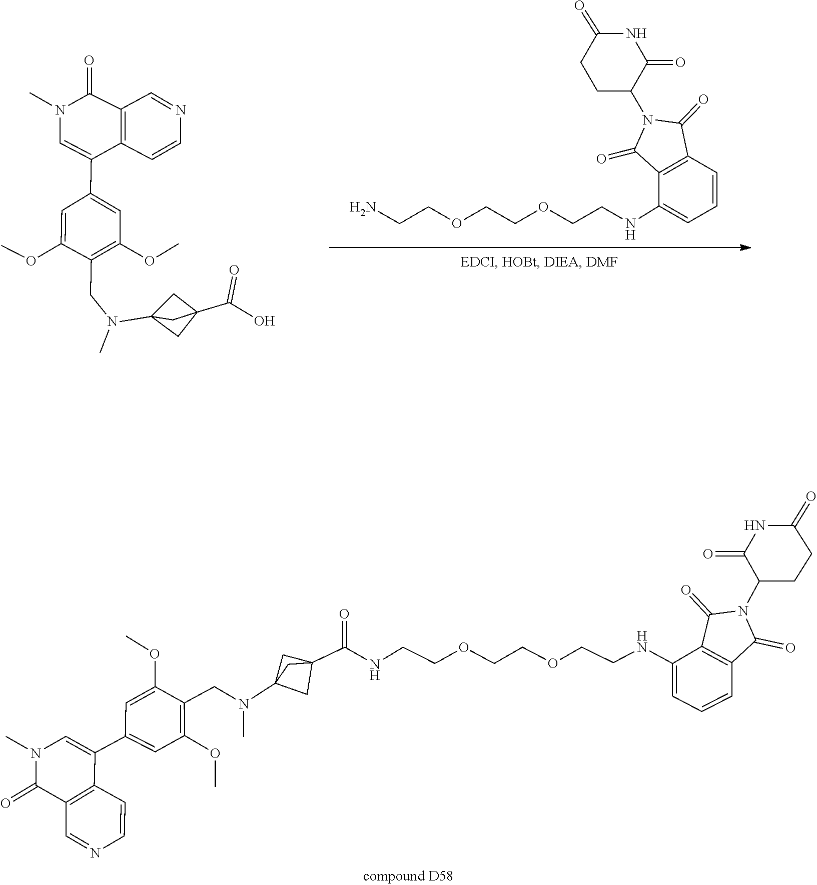 [SMILES: COc1cc(-c2cn(C)c(=O)c3cnccc23)cc(OC)c1CN(C)C12CC(C(=O)O)(C1)C2 ; NCCOCCOCCNc1cccc2c1C(=O)N(C1CCC(=O)NC1=O)C2=O ; COc1cc(-c2cn(C)c(=O)c3cnccc23)cc(OC)c1CN(C)C12CC(C(=O)NCCOCCOCCNc3cccc4c3C(=O)N(C3CCC(=O)NC3=O)C4=O)(C1)C2]

To a stirred mixture of 3-([[2,6-dimethoxy-4-(2-methyl-1-oxo-2,7-naphthyridin-4-yl)phenyl]methyl](methyl)amino)bicyclo[1.1.1]pentane-1-carboxylic acid (50.0 mg, 0.111 mmol, 1.00 equiv) in DMF (2.00 mL) was added EDCl (42.7 mg, 0.222 mmol, 2.00 equiv) and 4-([2-[2-(2-aminoethoxy)ethoxy]ethyl]amino)-2-(2,6-dioxopiperidin-3-yl)isoindole-1,3-dione (49.5 mg, 0.122 mmol, 1.10 equiv). The mixture was stirred at room temperature for 30 minutes, and then DIEA (71.9 mg, 0.556 mmol, 5.00 equiv) and 4-([2-[2-(2-aminoethoxy)ethoxy]ethyl]amino)-2-(2,6-dioxopiperidin-3-yl)isoindole-1,3-dione (9.9 mg, 0.024 mmol, 1.10 equiv) were added. After stirring at room temperature for 2 hours, without any additional work-up, the mixture was purified by Prep-HPLC (conditions: column, Phenomenex Gemini C6-Phenyl, 21.2*250 mm, 5 μm; mobile phase, Water (0.05% FA) and ACN (11% Phase B up to 21% in 28 minutes). This resulted in 3-([[2,6-dimethoxy-4-(2-methyl-1-oxo-2,7-naphthyridin-4-yl)phenyl]methyl](methyl)amino)-N-[2-[2-(2-[[2-(2,6-dioxopiperidin-3-yl)-1,3-dioxoisoindol-4-yl]amino]ethoxy)ethoxy]ethyl]bicyclo[1.1.1]pentane-1-carboxamide (10.5 mg, 10.6%) as a yellow solid. ¹H NMR (300 MHz, Methanol-d4) δ 9.53 (s, 1H), 8.69 (d, J=5.7 Hz, 1H), 7.75 (s, 1H), 7.64 (d, J=5.7 Hz, 1H), 7.55 (dd, J=8.6, 7.1 Hz, 1H), 7.07 (dd, J=19.4, 7.8 Hz, 2H), 6.74 (s, 2H), 5.07 (dd, J=12.3, 5.4 Hz, 1H), 3.88 (s, 6H), 3.77 (t, J=5.2 Hz, 2H), 3.73-3.63 (m, 9H), 3.59 (t, J=5.5 Hz, 2H), 3.53 (t, J=5.2 Hz, 2H), 3.41 (t, J=5.5 Hz, 2H), 2.90-2.68 (m, 3H), 2.27 (s, 3H), 2.20-2.06 (m, 7H). LCMS (ESI) m/z: [M+H]⁺=836.40.

### Example 65—Preparation of N-[3-([[2,6-dimethoxy-4-(2-methyl-1-oxo-2,7-naphthyridin-4-yl)phenyl]methyl](methyl)amino)bicyclo[1.1.1]pentan-1-yl]-3-[2-(2-[[2-(2,6-dioxopiperidin-3-yl)-1,3-dioxoisoindol-4-yl]ami no]ethoxy)ethoxy]propanamide formic acid (compound D59 formic acid)

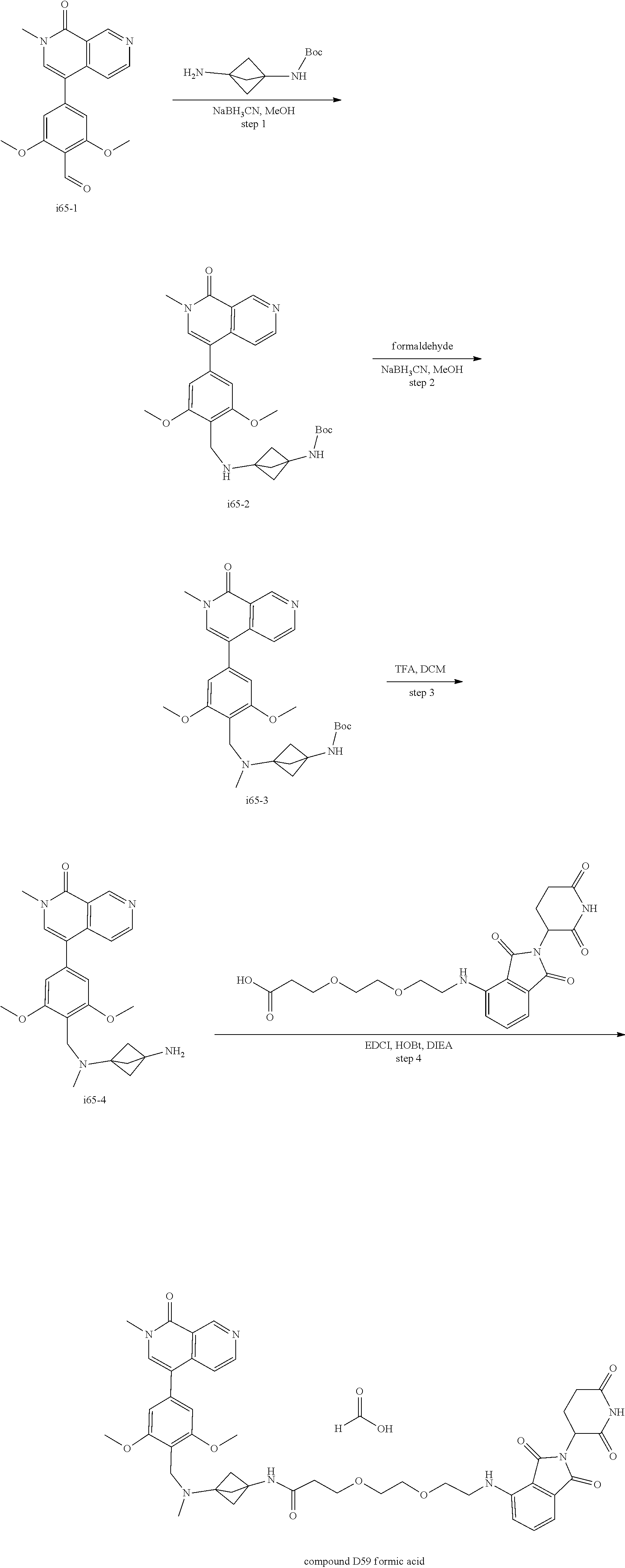

### Step 1: Preparation of tert-butyl (3-((2,6-dimethoxy-4-(2-methyl-1-oxo-1,2-dihydro-2,7-naphthyridin-4-yl)benzyl)amino)bicyclo[1.1.1]pentan-1-yl)carbamate (i65-2)

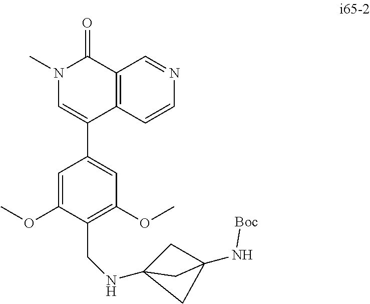 [SMILES: COc1cc(-c2cn(C)c(=O)c3cnccc23)cc(OC)c1CNC12CC(NC(=O)OC(C)(C)C)(C1)C2]

To a stirred solution of tert-butyl N-[3-aminobicyclo[1.1.1]pentan-1-yl]carbamate (134.49 mg, 0.678 mmol, 1.10 equiv) and tert-butyl N-[3-aminobicyclo[1.1.1]pentan-1-yl]carbamate (134.49 mg, 0.678 mmol, 1.00 equiv) in MeOH (3 mL) was added NaBH₃CN (77.50 mg, 1.233 mmol, 2.00 equiv) in portions at room temperature. The resulting mixture was stirred for 2 hours at room temperature. The crude resulting mixture was used in the next step directly without further purification. LCMS (ESI) m/z: [M+H]⁺=507.

### Step 2: Preparation of tert-butyl N-[3-([[2,6-dimethoxy-4-(2-methyl-1-oxo-2,7-naphthyridin-4-yl)phenyl]methyl](methyl)amino)bicyclo[1.1.1]pentan-1-yl]carbamate (i65-3)

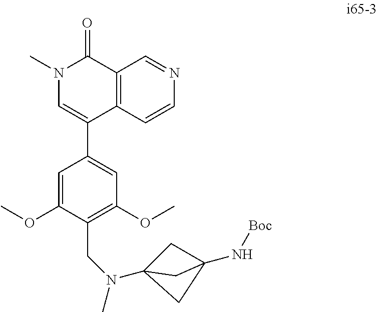 [SMILES: COc1cc(-c2cn(C)c(=O)c3cnccc23)cc(OC)c1CN(C)C12CC(NC(=O)OC(C)(C)C)(C1)C2]

To a stirred solution of the product from step 1 was added NaBH₃CN (49.62 mg, 0.790 mmol, 2.00 equiv) and formaldehyde (59.27 mg, 1.974 mmol, 5.00 equiv) in portions at room temperature. The resulting mixture was stirred for 2 hours at room temperature. Desired product could be detected by LCMS. The resulting mixture was concentrated under reduced pressure. The residue was purified by silica gel column chromatography, eluted with CH₂Cl₂/MeOH (8:1) to afford tert-butyl N-[3-([[2,6-dimethoxy-4-(2-methyl-1-oxo-2,7-naphthyridin-4-yl)phenyl]methyl](methyl)amino)bicyclo[1.1.1]pentan-1-yl]carbamate (146 mg, 71.03%) as a light yellow oil. LCMS (ESI) m/z: [M+H]⁺=521.

### Step 3: Preparation of 4-(4-(((3-aminobicyclo[1.1.1]pentan-1-yl)(methyl)amino)methyl)-3,5-dimethoxy phenyl)-2-methyl-2,7-naphthyridin-1(2H)-one (i65-4)

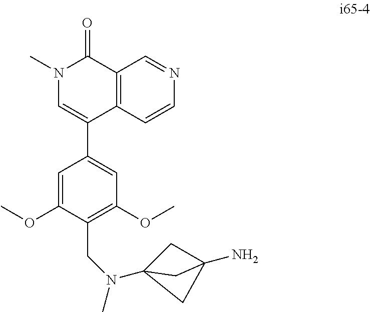 [SMILES: COc1cc(-c2cn(C)c(=O)c3cnccc23)cc(OC)c1CN(C)C12CC(N)(C1)C2]

To a stirred solution of tert-butyl N-[3-([[2,6-dimethoxy-4-(2-methyl-1-oxo-2,7-naphthyridin-4-yl)phenyl]methyl](methyl)amino)bicyclo[1.1.1]pentan-1-yl]carbamate (46.00 mg, 0.300 mmol, 1.00 equiv) in DCM was added TFA (1.00 mL) at room temperature. The resulting mixture was stirred for 2 hours at room temperature. The resulting mixture was concentrated under reduced pressure to afford 4-(4-(((3-aminobicyclo[1.1.1]pentan-1-yl)(methyl)amino)methyl)-3,5-dimethoxyphenyl)-2-methyl-2,7-naphthyridin-1(2H)-one (210 mg crude), which was used in the next step directly without further purification. LCMS (ESI) m/z: [M+H]⁺=421.

### Step 4: Preparation of N-[3-([[2,6-dimethoxy-4-(2-methyl-1-oxo-2,7-naphthyridin-4-yl)phenyl]methyl](methyl)amino)bicyclo[1.1.1]pentan-1-yl]-3-[2-(2-[[2-(2,6-dioxopiperidin-3-yl)-1,3-dioxoisoindol-4-yl]amino]ethoxy)ethoxy]propanamide formic acid (compound D59 formic acid)

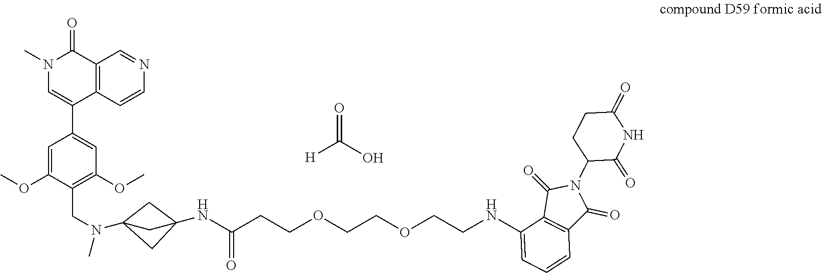 [SMILES: COc1cc(-c2cn(C)c(=O)c3cnccc23)cc(OC)c1CN(C)C12CC(NC(=O)CCOCCOCCNc3cccc4c3C(=O)N(C3CCC(=O)NC3=O)C4=O)(C1)C2 ; O=CO]

To a stirred solution of 4-[4-[([3-aminobicyclo[1.1.1]pentan-1-yl](methyl)amino)methyl]-3,5-dimethoxyphenyl]-2-methyl-2,7-naphthyridin-1-one (80.00 mg, 0.190 mmol, 1.00 equiv) and EDCl (72.94 mg, 0.380 mmol, 2.00 equiv) in DMF (1 mL) was added HOBT (51.41 mg, 0.380 mmol, 2.00 equiv) and DIEA (98.35 mg, 0.761 mmol, 4.00 equiv) in portions at room temperature. To the above mixture was added 3-[2-(2-[[2-(2, 6-dioxopiperidin-3-yl)-1,3-dioxoisoindol-4-yl]amino]ethoxy)ethoxy]propanoic acid (82.45 mg, 0.190 mmol, 1.00 equiv) at room temperature. The resulting mixture was stirred for additional overnight at room temperature. Desired product could be detected by LCMS. The crude product (78.2 mg) was purified by prep-HPLC (conditions: Xselect CSH F-Phenyl OBD column, 19*250, 5 μm; Mobile Phase A: Water (0.05% FA), Mobile Phase B:ACN; Flow rate:25 mL/minute; Gradient:15 B to 22 B in 17 minutes; 254/220 nm; R_{T}:15.32 minutes) to afford N-[3-([[2,6-dimethoxy-4-(2-methyl-1-oxo-2,7-naphthyridin-4-yl)phenyl]methyl](methyl)amino)bicyclo[1.1.1]pentan-1-yl]-3-[2-(2-[[2-(2,6-dioxopiperidin-3-yl)-1,3-dioxoisoindol-4-yl]amino]ethoxy)ethoxy]propanamide formic acid (23.7 mg, 14.13%) as a yellow solid. ¹H NMR (300 MHz, Methanol-d4) δ 9.53 (s, 1H), 8.70 (d, J=5.8 Hz, 1H), 8.20 (brs, 0.3H, FA), 7.78 (s, 1H), 7.63 (d, J=5.7 Hz, 1H), 7.55 (dd, J=8.6, 7.1 Hz, 1H), 7.11 (d, J=8.6 Hz, 1H), 7.03 (d, J=7.1 Hz, 1H), 6.85 (s, 2H), 5.08 (dd, J=12.3, 5.4 Hz, 1H), 4.20 (s, 2H), 3.95 (s, 6H), 3.78-3.63 (m, 11H), 3.52 (t, J=5.3 Hz, 2H), 2.99-2.66 (m, 6H), 2.52-2.34 (m, 8H), 2.18-2.08 (m, 1H). LCMS (ESI) m/z: [M+H]⁺=836.65.

### Example 66—Preparation of N-[3-([[2,6-dimethoxy-4-(2-methyl-1-oxo-2,7-naphthyridin-4-yl)phenyl]methyl](methyl)amino)bicyclo[1.1.1]pentan-1-yl]-6-[[2-(2,6-dioxopiperidin-3-yl)-1,3-dioxoisoindol-4-yl]oxy]hexanamide formic acid (compound D60 formic acid)

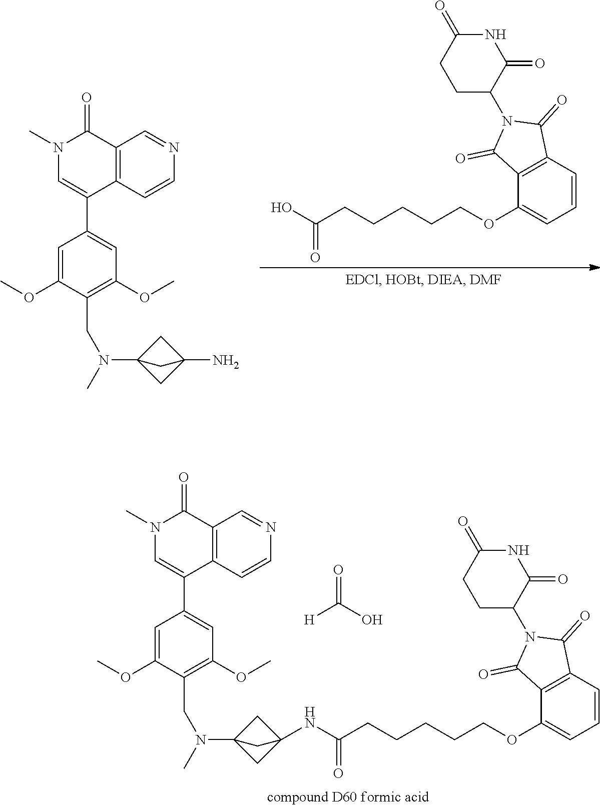 [SMILES: COc1cc(-c2cn(C)c(=O)c3cnccc23)cc(OC)c1CN(C)C12CC(N)(C1)C2 ; O=C(O)CCCCCOc1cccc2c1C(=O)N(C1CCC(=O)NC1=O)C2=O ; COc1cc(-c2cn(C)c(=O)c3cnccc23)cc(OC)c1CN(C)C12CC(NC(=O)CCCCCOc3cccc4c3C(=O)N(C3CCC(=O)NC3=O)C4=O)(C1)C2 ; O=CO]

To a stirred solution of 4-[4-[([3-aminobicyclo[1.1.1]pentan-1-yl](methyl)amino)methyl]-3,5-dimethoxy phenyl]-2-methyl-2,7-naphthyridin-1-one (80.00 mg, 0.190 mmol, 1.00 equiv) and EDCl (72.94 mg, 0.380 mmol, 2.00 equiv) in DMF (1 mL) was added HOBt (51.41 mg, 0.380 mmol, 2.00 equiv) at room temperature. To the above mixture was added DIEA (98.35 mg, 0.761 mmol, 4.00 equiv). The resulting mixture was stirred for overnight at room temperature. Without any additional work-up, the mixture was purified by prep-HPLC (conditions: Xselect CSH F-Phenyl OBD column, 19*250, 5 μm; Mobile Phase A: Water (0.05% FA), Mobile Phase B:ACN; Flow rate:25 mL/minute; Gradient:5 B to 35 B in 13 minutes; 254/220 nm; R_{T}:12.05 minutes) to afford N-[3-([[2,6-dimethoxy-4-(2-methyl-1-oxo-2,7-naphthyridin-4-yl)phenyl]methyl](methyl) amino)bicycle [1.1.1]pentan-1-yl]-6-[[2-(2,6-dioxopiperidin-3-yl)-1,3-dioxoisoindol-4-yl]oxy]hexanamide formic acid (14.9 mg, 9.36%) as a white solid. ¹H NMR (300 MHz, Methanol-d4) δ 9.53 (s, 1H), 8.69 (d, J=5.8 Hz, 1H), 8.44 (brs, 0.3H, FA), 7.84-7.74 (m, 2H), 7.64 (d, J=5.8 Hz, 1H), 7.46 (d, J=7.9 Hz, 2H), 6.78 (s, 2H), 5.10 (dd, J=12.0, 5.4 Hz, 1H), 4.25 (t, J=6.2 Hz, 2H), 3.91 (s, 8H), 3.72 (s, 3H), 2.91-2.67 (m, 3H), 2.39 (s, 3H), 2.29-2.20 (m, 8H), 2.18-2.08 (m, 1H), 1.91 (p, J=6.5 Hz, 2H), 1.73 (p, J=7.2 Hz, 2H), 1.60 (q, J=8.1 Hz, 2H). LCMS (ESI) m/z: [M+H]⁺=791.40.

### Example 67—Preparation of 5-(2-(4-(((1-(2,6-dimethoxy-4-(2-methyl-1-oxo-1,2-dihydro-2,7-naphthyridin-4-yl)benzyl)azetidin-3-yl)oxy)methyl)-1H-1,2,3-triazol-1-yl)ethoxy)-2-(2,6-dioxopiperidin-3-yl)isoindoline-1,3-dione (compound D61)

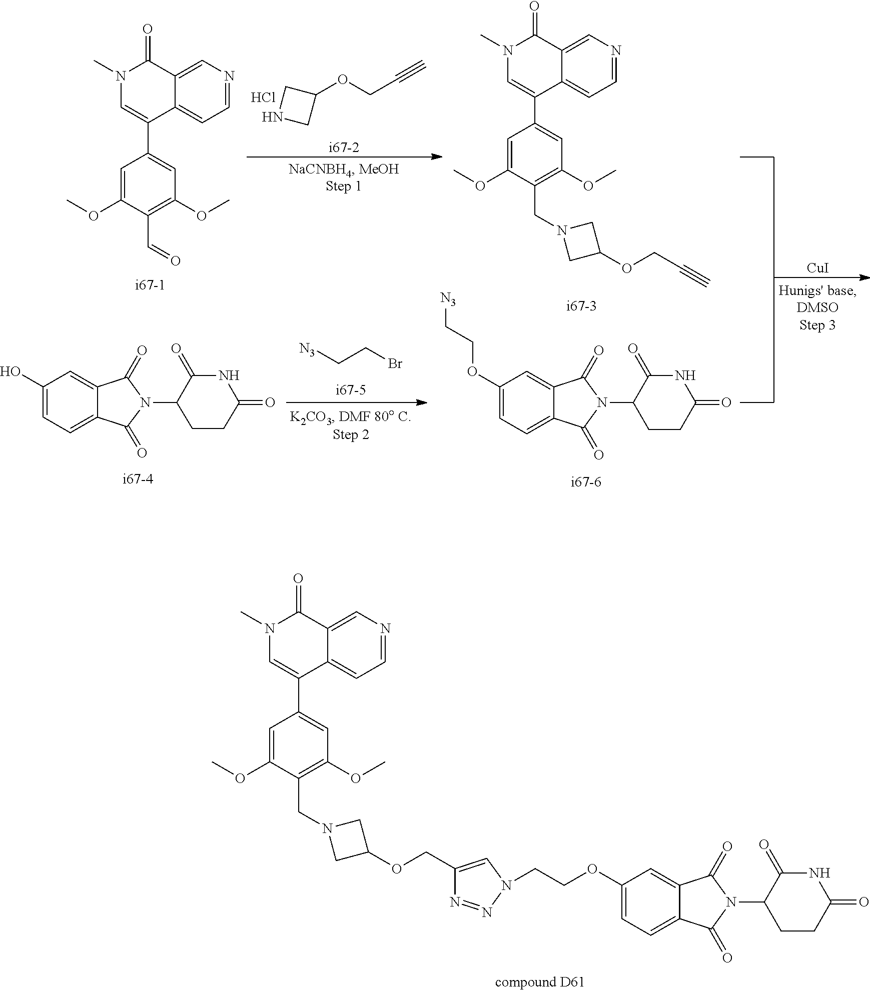

### Step 1: Preparation of 4-(3,5-dimethoxy-4-((3-(prop-2-yn-1-yloxy)azetidin-1-yl)methyl)phenyl)-2-methyl-2,7-naphthyridin-1(2H)-one (i67-3)

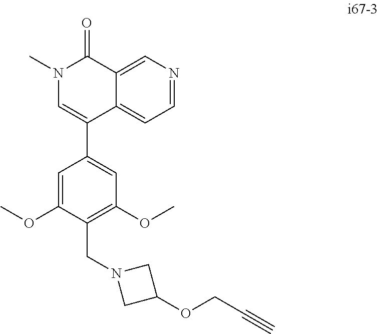 [SMILES: C#CCOC1CN(Cc2c(OC)cc(-c3cn(C)c(=O)c4cnccc34)cc2OC)C1]

To a stirred solution of 2,6-dimethoxy-4-(2-methyl-1-oxo-1,2-dihydro-2,7-naphthyridin-4-yl)benzaldehyde (500 mg, 1.54 mmol, 1.00 equiv) in MeOH (15 mL) was added NaBH₃CN (290 mg, 4.62 mmol, 3.00 equiv) and 3-(prop-2-yn-1-yloxy)azetidine hydrochloride (269 mg, 1.84 mmol, 1.20 equiv).

The resulting mixture was stirred for 2 hours at room temperature. Solvent was removed and the residue was purified by Flash column chromatography with EtOAc/PE (0-100%) to afford 4-(3,5-dimethoxy-4-((3-(prop-2-yn-1-yloxy)azetidin-1-yl)methyl)phenyl)-2-methyl-2,7-naphthyridin-1 (2H)-one (451 mg, 70%) as a solid. LCMS (ESI) m/z: [M+H]⁺=420.4.

### Step 2: Preparation of 5-(2-azidoethoxy)-2-(2,6-dioxopiperidin-3-yl)isoindoline-1,3-dione (i67-6)

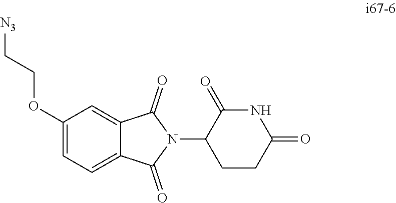 [SMILES: [N-]=[N+]=NCCOc1ccc2c(c1)C(=O)N(C1CCC(=O)NC1=O)C2=O]

2-(2,6-dioxopiperidin-3-yl)-5-hydroxyisoindoline-1,3-dione (500 mg, 1.82 mmol, 1.0 equiv) was dissolved in DMF (15 mL). Potassium carbonate was then added (753 mg, 545 mmol, 3 equiv) followed by potassium iodide (451 mg, 2.72 mmol, 1.5 equiv) and 1-azido-2-bromoethane (286 mg, 1.91 mmol, 1.05 equiv). The mixture was then heated to 80° C. and stirred for 2 hours. The solvent was then removed and Flash column chromatography with EtOAc/PE (0-100%), to afford 5-(2-azidoethoxy)-2-(2,6-dioxopiperidin-3-yl)isoindoline-1,3-dione (385 mg, 62%) as a solid. LCMS (ESI) m/z: [M+H]⁺=344.4.

### Step 3: Preparation of 5-(2-(4-(((1-(2,6-dimethoxy-4-(2-methyl-1-oxo-1,2-dihydro-2,7-naphthyridin-4-yl)benzyl)azetidin-3-yl)oxy)methyl)-1H-1,2,3-triazol-1-yl)ethoxy)-2-(2,6-dioxopiperidin-3-yl)isoindoline-1,3-dione (compound D61)

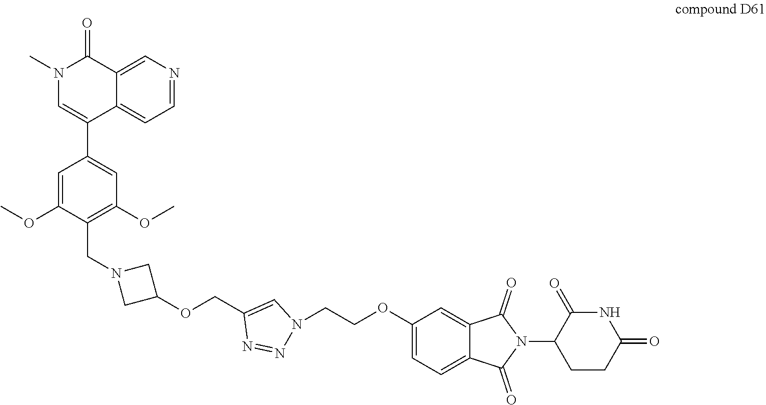 [SMILES: COc1cc(-c2cn(C)c(=O)c3cnccc23)cc(OC)c1CN1CC(OCc2cn(CCOc3ccc4c(c3)C(=O)N(C3CCC(=O)NC3=O)C4=O)nn2)C1]

5-(2-azidoethoxy)-2-(2,6-dioxopiperidin-3-yl)isoindoline-1,3-dione (20 mg, 0.0595 mmol, 1.0 equiv) and 4-(3,5-dimethoxy-4-((3-(prop-2-yn-1-yloxy)azetidin-1-yl)methyl)phenyl)-2-methyl-2,7-naphthyridin-1(2H)-one (25 mg, 0.0595 mmol, 1.0 equiv) were dissolved in DMSO (1 mL). Hünig; s base (0.020 mL, 0.119 mmol, 2 equiv) was then added followed by CuI (5.69 mg, 0.0297 mmol, 0.5 equiv). The mixture was stirred for 1 hour at room temperature. The solution was submitted directly for HPLC purification to give 5-(2-(4-(((1-(2,6-dimethoxy-4-(2-methyl-1-oxo-1,2-dihydro-2,7-naphthyridin-4-yl)benzyl)azetidin-3-yl)oxy)methyl)-1H-1,2,3-triazol-1-yl)ethoxy)-2-(2,6-dioxopiperidin-3-yl)isoindoline-1,3-dione (14.8 mg, 33%) as a solid. ¹H NMR (400 MHz, DMSO-d6) δ 11.08 (s, 1H), 9.42 (s, 1H), 8.70 (s, 1H), 8.18 (s, 1H), 7.85 (s, 1H), 7.80 (d, J=8.3 Hz, 1H), 7.52 (d, J=5.8 Hz, 1H), 7.46 (d, J=2.3 Hz, 1H), 7.31 (dd, J=8.3, 2.3 Hz, 1H), 6.69 (s, 2H), 5.09 (dd, J=12.8, 5.4 Hz, 1H), 4.79 (t, J=4.9 Hz, 2H), 4.60 (t, J=5.0 Hz, 2H), 4.39 (s, 2H), 4.00 (t, J=6.1 Hz, 1H), 3.83-3.76 (m, 1H), 3.76 (s, 6H), 3.57 (d, J=4.2 Hz, 5H), 2.93-2.84 (m, 1H), 2.83 (s, 3H), 2.68-2.63 (m, 2H), 2.59 (s, 1H), 2.54 (s, 1H), 2.08-1.95 (m, 2H). LCMS (ESI) m/z: [M+H]+=761.4.

### Example 68—Preparation of 5-(4-(((2,6-dimethoxy-4-(2-methyl-1-oxo-1,2-dihydro-2,7-naphthyridin-4-yl)benzyl)(methyl)amino)methyl)-1H-1,2,3-triazol-1-yl)-2-(2,6-dioxopiperidin-3-yl)isoindoline-1,3-dione (compound D62)

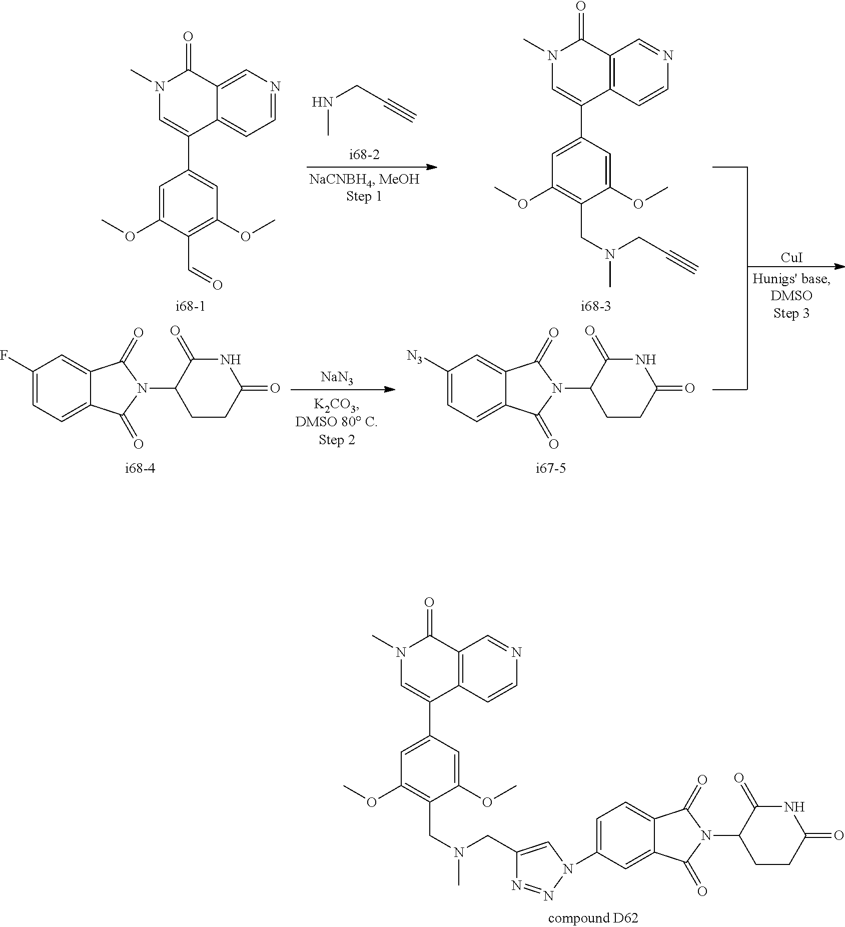

### Step 1: Preparation of 4-(3,5-dimethoxy-4-((methyl(prop-2-yn-1-yl)amino)methyl)phenyl)-2-methyl-2,7-naphthyridin-1(2H)-one (i68-3)

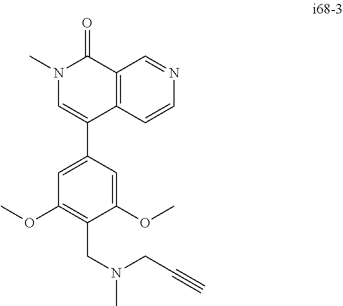 [SMILES: C#CCN(C)Cc1c(OC)cc(-c2cn(C)c(=O)c3cnccc23)cc1OC]

To a stirred solution of 2,6-dimethoxy-4-(2-methyl-1-oxo-1,2-dihydro-2,7-naphthyridin-4-yl)benzaldehyde (500 mg, 1.54 mmol, 1.00 equiv) in MeOH (15 mL) was added NaBH₃CN (290 mg, 4.62 mmol, 3.00 equiv) and N-methylprop-2-yn-1-amine (127 mg, 1.84 mmol, 1.20 equiv). The resulting mixture was stirred for 2 hours at room temperature. Solvent was removed and the residue was purified by Flash column chromatography with EtOAc/PE (0-100%), to afford 4-(3,5-dimethoxy-4-((methyl(prop-2-yn-1-yl)amino)methyl)phenyl)-2-methyl-2,7-naphthyridin-1(2H)-one (390 mg, 67%) as a solid. LCMS (ESI) m/z: [M+H]⁺=378.7.

### Step 2: Preparation of 5-azido-2-(2,6-dioxopiperidin-3-yl)isoindoline-1,3-dione (i68-5)

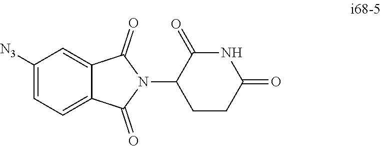 [SMILES: [N-]=[N+]=Nc1ccc2c(c1)C(=O)N(C1CCC(=O)NC1=O)C2=O]

2-(2,6-dioxopiperidin-3-yl)-5-fluoroisoindoline-1,3-dione (500 mg, 1.81 mmol, 1.0 equiv) was dissolved in DMSO (5 mL). Hunig's base was then added (0.944 mL, 5.43 mmol, 3 equiv) followed by sodium azide (176 mg, 2.71 mmol, 1.5 equiv) and 1-azido-2-bromoethane (286 mg, 1.91 mmol, 1.05 equiv). The mixture was then heated to 50° C. and stirred for 2 hours. The solution was then loaded directly onto silica gel and purified over silica gel with EtOAc/PE (0-100%) to afford 5-azido-2-(2,6-dioxopiperidin-3-yl)isoindoline-1,3-dione (480 mg, 89%) as a solid. LCMS (ESI) m/z: [M+H]⁺=300.1.

### Step 3: 5-(4-(((2,6-dimethoxy-4-(2-methyl-1-oxo-1,2-dihydro-2,7-naphthyridin-4-yl)benzyl)(methyl)amino)methyl)-1H-1,2,3-triazol-1-yl)-2-(2,6-dioxopiperidin-3-yl)isoindoline-1,3-dione (compound D62)

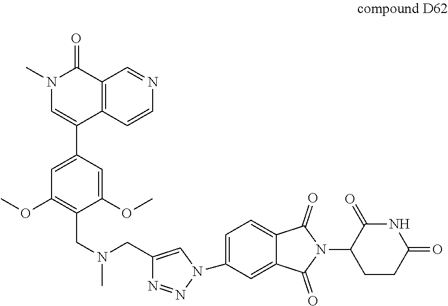 [SMILES: COc1cc(-c2cn(C)c(=O)c3cnccc23)cc(OC)c1CN(C)Cc1cn(-c2ccc3c(c2)C(=O)N(C2CCC(=O)NC2=O)C3=O)nn1]

5-(2-azidoethoxy)-2-(2,6-dioxopiperidin-3-yl)isoindoline-1,3-dione (19.8 mg, 0.0662 mmol, 1.0 equiv) and 4-(3,5-dimethoxy-4-((3-(prop-2-yn-1-yloxy)azetidin-1-yl)methyl)phenyl)-2-methyl-2,7-naphthyridin-1(2H)-one (25 mg, 0.0662 mmol, 1.0 equiv) were dissolved in DMSO (1 mL). Hunig's base (0.023 mL, 0.132 mmol, 2 equiv) was then added followed by CuI (6.3 mg, 0.0279 mmol, 0.5 equiv). The mixture was stirred for 1 hour at room temperature. The solution was submitted directly for HPLC purification to 5-(4-(((2,6-dimethoxy-4-(2-methyl-1-oxo-1,2-dihydro-2,7-naphthyridin-4-yl)benzyl)(methyl)amino)methyl)-1H-1,2,3-triazol-1-yl)-2-(2,6-dioxopiperidin-3-yl)isoindoline-1,3-dione (12.3 mg, 28%) as a solid. ¹H NMR (400 MHz, DMSO-d6) δ 11.14 (s, 1H), 9.43 (s, 1H), 9.03 (s, 1H), 8.70 (d, J=5.7 Hz, 1H), 8.52-8.45 (m, 2H), 8.13 (d, J=14.2 Hz, 1H), 7.85 (s, 1H), 7.54 (d, J=5.7 Hz, 1H), 6.73 (s, 2H), 5.20 (dd, J=12.9, 5.3 Hz, 1H), 3.78 (s, 6H), 3.58 (s, 3H), 2.96-2.83 (m, 1H), 2.65-2.58 (m, 1H), 2.58-2.50 (m, 1H), 2.22 (s, 5H), 2.13-2.03 (m, 1H). LCMS (ESI) m/z: [M+H]+=675.4.

### Example 69—Preparation of 5-(4-(4-(((1-(2,6-dimethoxy-4-(2-methyl-1-oxo-1,2-dihydro-2,7-naphthyridin-4-yl)benzyl)piperidin-3-yl)oxy)methyl)-1H-1,2,3-triazol-1-yl)butoxy)-2-(2,6-dioxopiperidin-3-yl)isoindoline-1,3-dione (compound D63)

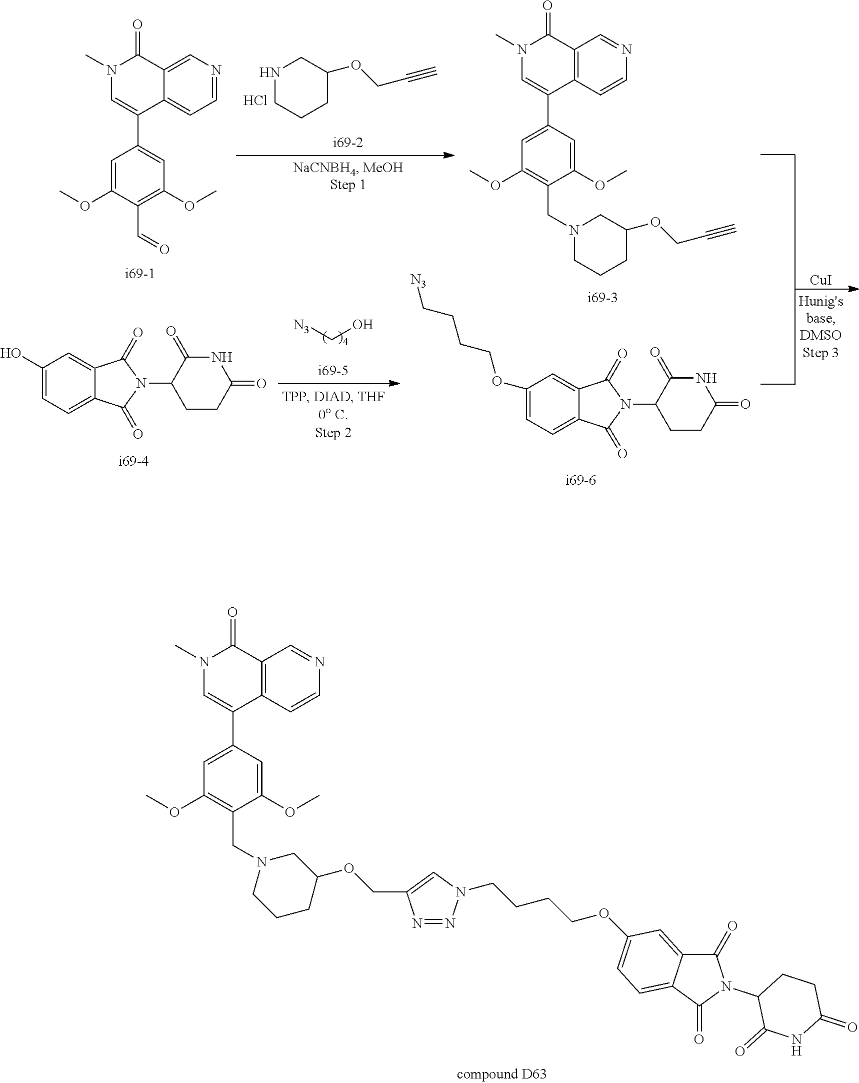

### Step 1: Preparation of 4-(3,5-dimethoxy-4-((3-(prop-2-yn-1-yloxy)piperidin-1-yl)methyl)phenyl)-2-methyl-2,7-naphthyridin-1(2H)-one (i69-3)

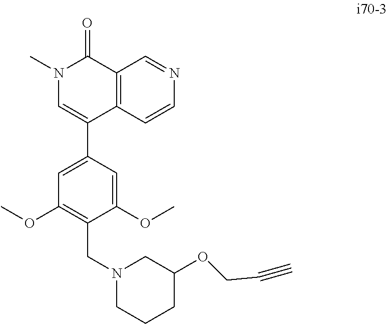 [SMILES: C#CCOC1CCCN(Cc2c(OC)cc(-c3cn(C)c(=O)c4cnccc34)cc2OC)C1]

To a stirred solution of 2,6-dimethoxy-4-(2-methyl-1-oxo-1,2-dihydro-2,7-naphthyridin-4-yl)benzaldehyde (500 mg, 1.54 mmol, 1.00 equiv) in MeOH (15 mL) was added NaBH₃CN (290 mg, 4.62 mmol, 3.00 equiv) and 3-(prop-2-yn-1-yloxy)piperidine hydrochloride (321 mg, 1.84 mmol, 1.20 equiv). The resulting mixture was stirred for 2 hours at room temperature. Solvent was removed and the residue was purified by Flash column chromatography with EtOAc/PE (0-100%) to afford 4-(3,5-dimethoxy-4-((3-(prop-2-yn-1-yloxy)piperidin-1-yl)methyl)phenyl)-2-methyl-2,7-naphthyridin-1(2H)-one (323 mg, 47%) as a solid. LCMS (ESI) m/z: [M+H]⁺=448.5.

### Step 2: Preparation of 5-(4-azidobutoxy)-2-(2,6-dioxopiperidin-3-yl)isoindoline-1,3-dione (i69-6)

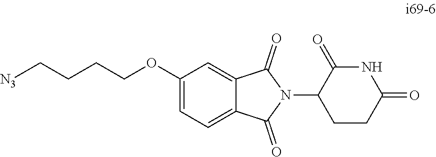 [SMILES: [N-]=[N+]=NCCCCOc1ccc2c(c1)C(=O)N(C1CCC(=O)NC1=O)C2=O]

2-(2,6-dioxopiperidin-3-yl)-5-hydroxyisoindoline-1,3-dione (500 mg, 1.82 mmol, 1.0 equiv) was dissolved in THF (18 mL). Triphenylphosphine was then added (571 mg, 2.18 mmol, 1.2 equiv) followed by 4-azidobutan-1-ol (246 mg, 2.91 mmol, 1.05 equiv). The solution was cooled to O ° C. and 1-diisopropyl azodicarboxylate (358 mL, 1.82 mmol, 1.0 equiv) was added. The mixture was then warmed to room temperature and stirred for 2 hours. Water was added and the reaction extracted 3 times with ethyl acetate. The organics were dried over MgSO4, filtered, and evaporated. The resulting oil was columned over silica gel with EtOAc/PE (0-100%), to afford 5-(4-azidobutoxy)-2-(2,6-dioxopiperidin-3-yl)isoindoline-1,3-dione (391 mg, 56%) as a solid. LCMS (ESI) m/z: [M+H]⁺=372.4.

### Step 3: 5-(4-(4-(((1-(2,6-dimethoxy-4-(2-methyl-1-oxo-1,2-dihydro-2,7-naphthyridin-4-yl)benzyl)piperidin-3-yl)oxy)methyl)-1H-1,2,3-triazol-1-yl)butoxy)-2-(2,6-dioxopiperidin-3-yl)isoindoline-1,3-dione (compound D63)

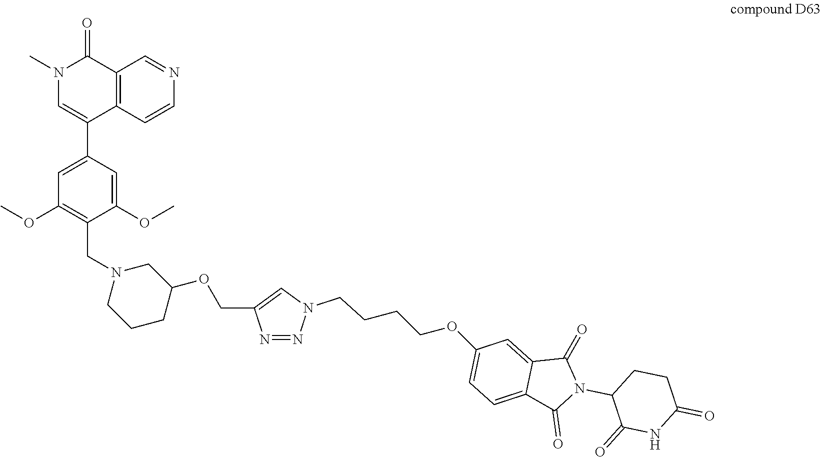 [SMILES: COc1cc(-c2cn(C)c(=O)c3cnccc23)cc(OC)c1CN1CCCC(OCc2cn(CCCCOc3ccc4c(c3)C(=O)N(C3CCC(=O)NC3=O)C4=O)nn2)C1]

5-(4-azidobutoxy)-2-(2,6-dioxopiperidin-3-yl)isoindoline-1,3-dione (21.5 mg, 0.0558 mmol, 1.0 equiv) and 4-(3,5-dimethoxy-4-((3-(prop-2-yn-1-yloxy)azetidin-1-yl)methyl)phenyl)-2-methyl-2,7-naphthyridin-1(2H)-one (25 mg, 0.0558 mmol, 1.0 equiv) were dissolved in DMSO (1 mL). Hunig's base (0.0192 mL, 0.111 mmol, 2 equiv) was then added followed by CuI (5.31 mg, 0.0279 mmol, 0.5 equiv). The mixture was stirred for 1 hour at room temperature. The solution was submitted directly for HPLC purification to give 5-(4-(4-(((1-(2,6-dimethoxy-4-(2-methyl-1-oxo-1,2-dihydro-2,7-naphthyridin-4-yl)benzyl)piperidin-3-yl)oxy)methyl)-1H-1,2,3-triazol-1-yl)butoxy)-2-(2,6-dioxopiperidin-3-yl)isoindoline-1,3-dione (6.2 mg, 12%) as a solid. ¹H NMR (400 MHz, DMSO-d6) δ 11.08 (s, 1H), 8.09 (s, 1H), 7.87 (s, 1H), 7.80 (d, J=8.3 Hz, 1H), 7.77-7.56 (m, 1H), 7.39 (d, J=2.3 Hz, 1H), 7.31 (dd, J=8.3, 2.3 Hz, 1H), 6.73 (s, 2H), 6.58-6.39 (m, 1H), 5.09 (dd, J=12.9, 5.4 Hz, 1H), 4.59-4.47 (m, 2H), 4.42 (t, J=7.0 Hz, 2H), 4.17 (t, J=6.4 Hz, 2H), 3.80 (s, 6H), 3.70 (s, 2H), 3.58 (s, 2H), 3.00 (s, 1H), 2.87 (ddd, J=17.4, 14.1, 5.4 Hz, 1H), 2.74 (s, 1H), 2.68-2.63 (m, OH), 2.62-2.50 (m, 2H), 2.33-2.27 (m, 1H), 2.05 (s, 3H), 2.03-1.93 (m, 1H), 1.97-1.78 (m, OH), 1.71 (q, J=6.7 Hz, 3H), 1.40 (s, 1H). LCMS (ESI) m/z: [M+H]⁺=817.2.

### Example 70—Preparation of 5-[2-(9-[[2,6-dimethoxy-4-(2-methyl-1-oxo-2,7-naphthyridin-4-yl)phenyl]methyl]-1-ox a-4,9-diazaspiro[5.5]undecan-4-yl)-2-oxoethoxy]-2-(2,6-dioxopiperidin-3-yl)isoindole-1,3-dione formic acid (compound D64 formic acid)

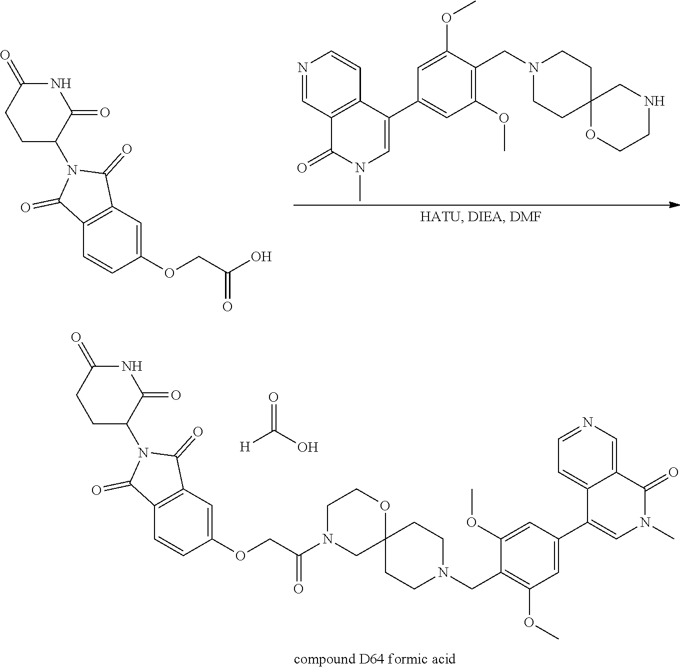 [SMILES: O=C(O)COc1ccc2c(c1)C(=O)N(C1CCC(=O)NC1=O)C2=O ; COc1cc(-c2cn(C)c(=O)c3cnccc23)cc(OC)c1CN1CCC2(CC1)CNCCO2 ; COc1cc(-c2cn(C)c(=O)c3cnccc23)cc(OC)c1CN1CCC2(CC1)CN(C(=O)COc1ccc3c(c1)C(=O)N(C1CCC(=O)NC1=O)C3=O)CCO2 ; O=CO]

To a stirred solution of [[2-(2,6-dioxopiperidin-3-yl)-1,3-dioxoisoindol-5-yl]oxy]acetic acid (21.46 mg, 0.065 mmol, 1.00 equiv) and 4-(3,5-dimethoxy-4-[1-oxa-4,9-diazaspiro[5.5]undecan-9-ylmethyl]phenyl)-2-methyl-2,7-naphthyridin-1-one(30.00 mg, 0.065 mmol, 1.00 equiv) in DMF (1 mL) was added HATU (49.11 mg, 0.129 mmol, 2.00 equiv) and DIEA (33.38 mg, 0.258 mmol, 4.00 equiv) at room temperature. The mixture was stirred at room temperature for 16 hours. Without any additional work-up, the mixture was purified by prep-HPLC (conditions: SunFire Prep C18 OBD Column, 19×150 mm 5 μm nm; Mobile Phase A: Water (0.1% FA), Mobile Phase B:ACN; Flow rate:25 mL/minute; Gradient:8 B to 33 B in 10 minutes; 254/220 nm; R_{T}: 8.05 minutes) to afford 5-[2-(9-[[2,6-dimethoxy-4-(2-methyl-1-oxo-2,7-naphthyridin-4-yl)phenyl]methyl]-1-oxa-4,9-diazaspiro[5.5]undecan-4-yl)-2-oxoethoxy]-2-(2,6-dioxopiperidin-3-yl)isoindole-1,3-dione formic acid as a white gum (6.8 mg, 12.77%). ¹H NMR (400 MHz, Methanol-d4) δ 9.54 (s, 1H), 8.69 (d, J=5.8 Hz, 1H), 8.56 (brs, 0.3H, FA), 7.84 (dd, J=8.4, 2.3 Hz, 1H), 7.76 (d, J=2.5 Hz, 1H), 7.67-7.62 (m, 1H), 7.45 (t, J=2.8 Hz, 1H), 7.40 (dd, J=8.3, 2.2 Hz, 1H), 6.81 (d, J=4.5 Hz, 2H), 5.16-5.00 (m, 3H), 4.18-3.98 (m, 2H), 3.92 (d, J=1.6 Hz, 6H), 3.85-3.75 (m, 2H), 3.72 (s, 3H), 3.67-3.59 (m, 2H), 3.56-3.45 (m, 2H), 3.11-2.91 (m, 3H), 2.90-2.64 (m, 4H), 2.18-2.09 (m, 1H), 2.08-1.91 (m, 2H), 1.85-1.69 (m, 2H). LCMS (ESI) m/z: [M+H]⁺=779.55.

### Example 71—Preparation of N-[[2-(4-[[2,6-dimethoxy-4-(2-methyl-1-oxo-2,7-naphthyridin-4-yl)phenyl]methyl]piperazine-1-carbonyl)cyclopropyl]methyl]-2-[[2-(2,6-dioxopiperidin-3-yl)-1,3-dioxoisoindol-4-yl]oxy]acetamide (compound D65)

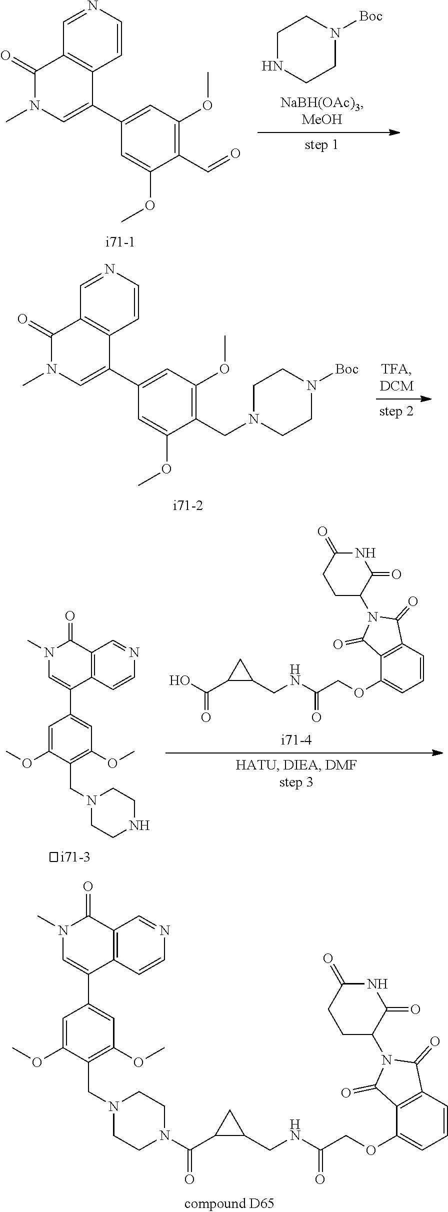 [SMILES: COc1cc(-c2cn(C)c(=O)c3cnccc23)cc(OC)c1C=O ; CC(C)(C)OC(=O)N1CCNCC1 ; COc1cc(-c2cn(C)c(=O)c3cnccc23)cc(OC)c1CN1CCN(C(=O)OC(C)(C)C)CC1 ; COc1cc(-c2cn(C)c(=O)c3cnccc23)cc(OC)c1CN1CCNCC1 ; O=C(COc1cccc2c1C(=O)N(C1CCC(=O)NC1=O)C2=O)NCC1CC1C(=O)O ; COc1cc(-c2cn(C)c(=O)c3cnccc23)cc(OC)c1CN1CCN(C(=O)C2CC2CNC(=O)COc2cccc3c2C(=O)N(C2CCC(=O)NC2=O)C3=O)CC1]

### Step 1: Preparation of tert-butyl 4-[[2,6-dimethoxy-4-(2-methyl-1-oxo-2,7-naphthyridin-4-yl)phenyl]methyl]piperazine-1-carboxylate (i71-2)

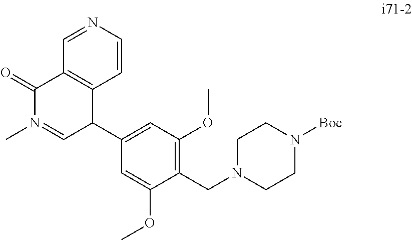

To a stirred solution of 2,6-dimethoxy-4-(2-methyl-1-oxo-2,7-naphthyridin-4-yl)benzaldehyde(100.00 mg, 0.308 mmol, 1.00 equiv) and tert-butyl piperazine-1-carboxylate(86.14 mg, 0.462 mmol, 1.50 equiv) in MeOH (1 mL) was added NaBH(OAc)₃ (261.38 mg, 1.233 mmol, 4.00 equiv) at room temperature. The resulting mixture was stirred for 2 hours at room temperature. The reaction mixture was filtered, and the filtrate was concentrated under reduced pressure. The crude product was purified by flash silica chromatography, elution gradient 0 to 60% EtOAc in petroleum ether. Pure fractions were evaporated to dryness to afford product tert-butyl 4-[[2,6-dimethoxy-4-(2-methyl-1-oxo-2,7-naphthyridin-4-yl)phenyl]methyl]piperazine-1-carboxylate (115 mg, 75.4%) as a yellow gum. LCMS (ESI) m/z: [M+H]⁺=495.

### Step 2: Preparation of 4-[3,5-dimethoxy-4-(piperazin-1-ylmethyl)phenyl]-2-methyl-2,7-naphthyridin-1-one (i71-3)

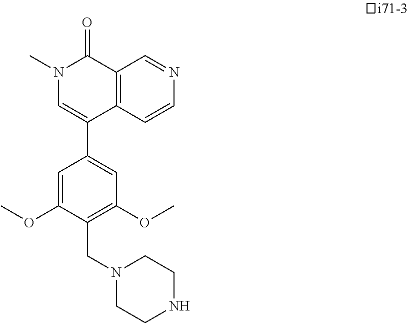 [SMILES: COc1cc(-c2cn(C)c(=O)c3cnccc23)cc(OC)c1CN1CCNCC1]

A solution of tert-butyl 4-[[2,6-dimethoxy-4-(2-methyl-1-oxo-2,7-naphthyridin-4-yl)phenyl]methyl]piperazine-1-carboxylate (115.00 mg) and TFA (1.00 mL) in DCM (1.00 mL) was stirred at room temperature for 2 hours. The reaction mixture was concentrated under reduced pressure to afford 4-[3,5-dimethoxy-4-(pipera zin-1-ylmethyl)phenyl]-2-methyl-2,7-naphthyridin-1-one (305 mg, crude), which was used directly without further purification. LCMS (ESI) m/z: [M+H]⁺=395.

### Step 3: Preparation of N-[[2-(4-[[2,6-dimethoxy-4-(2-methyl-1-oxo-2,7-naphthyridin-4-yl)phenyl]methyl]piperazine-1-carbonyl)cyclopropyl]methyl]-2-[[2-(2,6-dioxopiperidin-3-yl)-1,3-dioxoisoindol-4-yl]oxy]acetamide (compound D65)

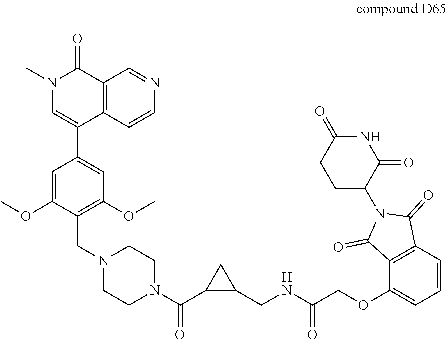

To a stirred mixture of 4-[3,5-dimethoxy-4-(piperazin-1-ylmethyl)phenyl]-2-methyl-2,7-naphthyridin-1-one (22.05 mg, 0.056 mmol, 1.20 equiv) and 2-[(2-[[2-(2,6-dioxopiperidin-3-yl)-1,3-dioxoisoindol-4-yl]oxy]acetamido)methyl]cyclopropane-1-carboxylic acid (20.00 mg, 0.047 mmol, 1.00 equiv) in DMF (1 mL) was added HATU (35.42 mg, 0.093 mmol, 2.00 equiv) and DIEA (12.04 mg, 0.093 mmol, 2.00 equiv) at room temperature. Without any additional work-up, the mixture was purified by prep-HPLC (conditions: XBridge Shield RP18 OBD Column, 30*150 mm, 5 μm; Mobile Phase A: Water (0.1% FA), Mobile Phase B:ACN; Flow rate:25 mL/minute; Gradient:13 B to 22 B in 12 minutes; 254/220 nm; R_{T}: 9.45 minutes). Pure fractions were evaporated to dryness to afford N-[[2-(4-[[2,6-dimethoxy-4-(2-methyl-1-oxo-2,7-naphthyridin-4-yl)phenyl]methyl]piperazine-1-carbonyl)cyclopropyl]meth-yl]-2-[[2-(2,6-dioxopiperidin-3-yl)-1,3-dioxoisoindol-4-yl]oxy]acetamide (12.4 mg, 33.04%) as a white solid. ¹H NMR (400 MHz, Methanol-d4) δ 9.52 (s, 1H), 8.68 (dd, J=5.8, 1.8 Hz, 1H), 8.43 (brs, 0.5H, FA), 7.81 (ddd, J=8.4, 7.3, 3.5 Hz, 1H), 7.75 (d, J=3.7 Hz, 1H), 7.65-7.61 (m, 1H), 7.54 (dd, J=6.9, 1.6 Hz, 1H), 7.45 (dd, J=8.3, 2.5 Hz, 1H), 6.76 (d, J=2.5 Hz, 2H), 5.19-5.11 (m, 1H), 4.80-4.68 (m, 2H), 3.93-3.81 (m, 9H), 3.78-3.68 (m, 5H), 3.51-3.35 (m, 2H), 3.29-3.16 (m, 1H), 2.93-2.67 (m, 7H), 2.21-2.06 (m, 2H), 1.72-1.60 (m, 1H), 1.21-1.12 (m, 1H), 1.09-0.99 (m, 1H). LCMS (ESI) m/z: [M+H]⁺=806.70.

### Example 72—Preparation of N-[[2-(4-[[2,6-dimethoxy-4-(2-methyl-1-oxo-2,7-naphthyridin-4-yl)phenyl]methyl]piperazine-1-carbonyl)cyclopropyl]methyl]-2-[[2-(2,6-dioxopiperidin-3-yl)-1,3-dioxoisoindol-5-yl]oxy]acetamide formic acid (compound D66 formic acid)

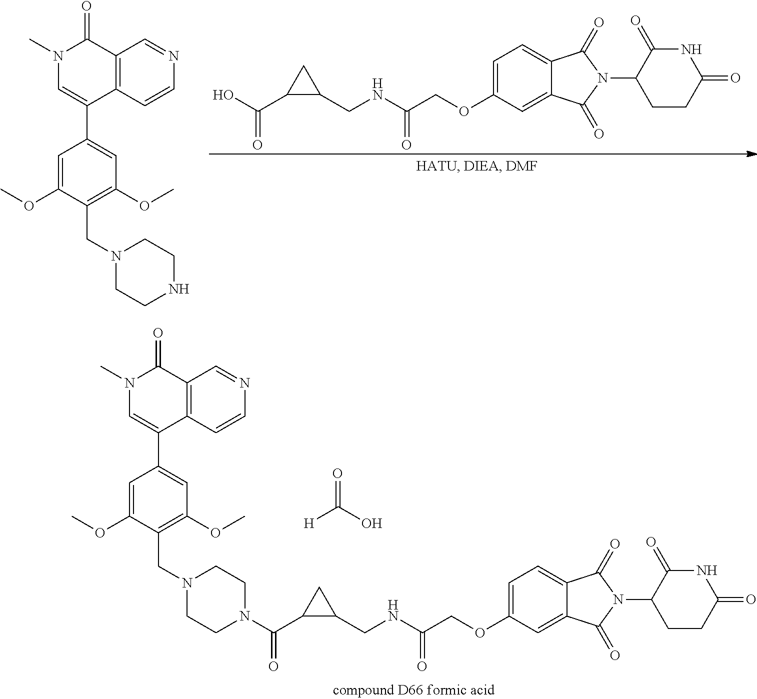

To a stirred mixture of 4-[3,5-dimethoxy-4-(piperazin-1-ylmethyl)phenyl]-2-methyl-2,7-naphthyridin-1-one (22.05 mg, 0.056 mmol, 1.20 equiv) and 2-[(2-[[2-(2,6-dioxopiperidin-3-yl)-1,3-dioxoisoindol-5-yl]oxy]acetamido)methyl]cyclopropane-1-carboxylic acid (20.00 mg, 0.047 mmol, 1.00 equiv) in DMF (1 mL) was added HATU (35.42 mg, 0.093 mmol, 2.00 equiv) and DIEA (12.04 mg, 0.093 mmol, 2.00 equiv) at room temperature. Without any additional work-up, the mixture was purified by prep-HPLC (conditions: XBridge Shield RP18 OBD Column, 19*250 mm, 10 μm; Mobile Phase A: Water (0.1% FA), Mobile Phase B:ACN; Flow rate:25 mL/minute; Gradient:13 B to 22 B in 12 minutes; 254/220 nm; R_{T}: 10.22 minutes). Pure fractions were evaporated to dryness to afford N-[[2-(4-[[2,6-dimeth oxy-4-(2-methyl-1-oxo-2,7-naphthyridin-4-yl)phenyl]methyl]piperazine-1-carbonyl)cyclopropyl]methyl]-2-[[2-(2,6-dioxopiperidin-3-yl)-1,3-dioxoisoindol-5-yl]oxy]acetamide (7.4 mg, 19.18%) as a white solid. ¹H NMR (400 MHz, Methanol-d4) δ 9.52 (s, 1H), 8.68 (d, J=5.8 Hz, 1H), 8.46 (brs, 1.0H, FA), 7.82 (d, J=8.3 Hz, 1H), 7.73 (s, 1H), 7.63 (d, J=5.8 Hz, 1H), 7.45 (d, J=2.3 Hz, 1H), 7.40 (dd, J=8.3, 2.3 Hz, 1H), 6.78 (s, 2H), 5.11 (dd, J=12.4, 5.4 Hz, 1H), 4.68 (s, 2H), 3.95 (s, 2H), 3.89 (s, 6H), 3.81 (s, 2H), 3.70 (s, 3H), 3.63 (s, 1H), 3.42-3.34 (m, 2H), 3.29-3.20 (m, 1H), 2.94-2.67 (m, 7H), 2.18-2.09 (m, 1H), 2.09-2.00 (m, 1H), 1.62 (q, J=7.5 Hz, 1H), 1.11 (q, J=5.5 Hz, 1H), 1.01 (td, J=8.1, 4.5 Hz, 1H). LCMS (ESI) m/z: [M+H]⁺=806.40.

### Example 73—Preparation of 1-[[2,6-dimethoxy-4-(2-methyl-1-oxo-1,2-dihydro-2,7-naphthyridin-4-yl)phenyl]methyl]-N-(6-[[2-(2,6-dioxopiperidin-3-yl)-1,3-dioxo-2,3-dihydro-1H-isoindol-5-yl]amino]hexyl) azetidine-3-sulfonamide formic acid (compound D67 formic acid)

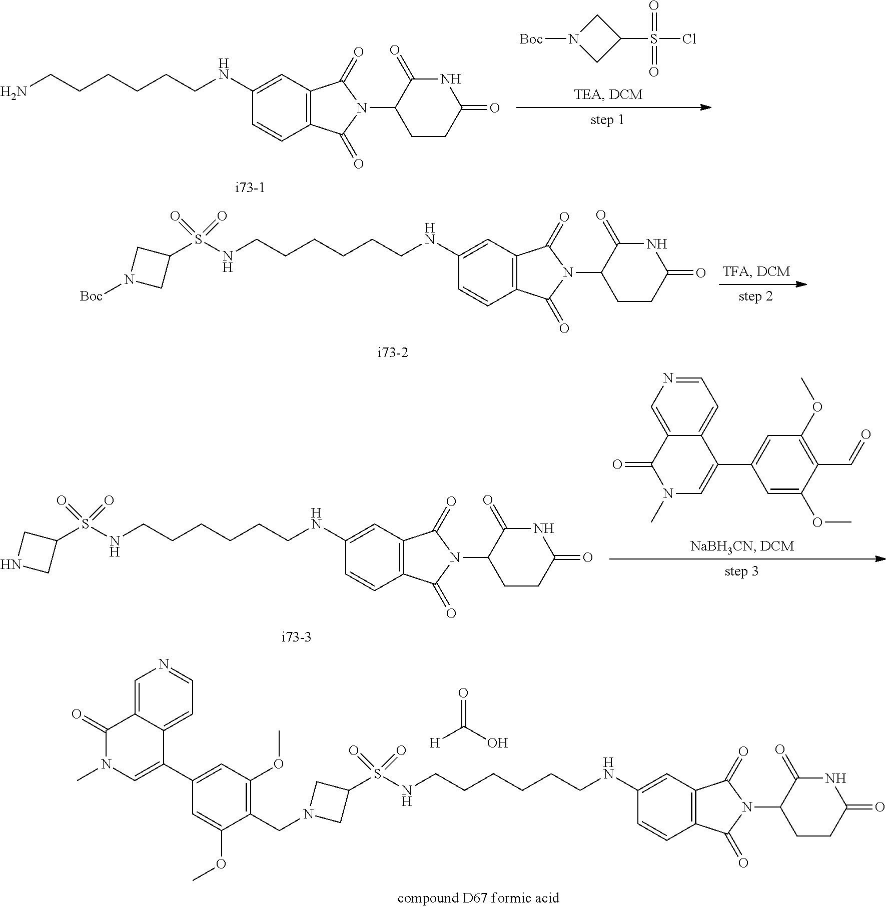

### Step 1: Preparation of tert-butyl-3-[(6-((2-(2,6-dioxopiperidin-3-yl)-1,3-dioxoisoindol-5-yl]amino]hexyl) sulfamoylazetidine-1-carboxylate (i73-2)

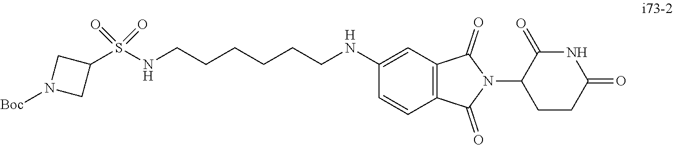

To a stirred mixture of 5-[(6-aminohexyl)amino]-2-(2,6-dioxopiperidin-3-yl)isoindole-1-3-dione(60.00 mg, 0.161 mmol, 1.00 equiv) and tert-butyl 3-(chlorosulfonyl)azetidine-1-carboxylate(102.99 mg, 0.403 mmol, 2.50 equiv) in DCM (2.00 mL) was added TEA (48.91 mg, 0.483 mmol, 3.00 equiv). After stirring for 1.5 hours at room temperature, the resulting mixture was concentrated under reduced pressure. The residue was purified by Prep-TLC (CH₂Cl₂/EA=1:2) to afford tert-butyl-3-[(6-[[2-(2,6-dioxopiperidin-3-yl)-1,3-dioxoisoindol-5-yl]amino]hexyl)sulfamoyl]azetidine-1-carboxylate (61.8 mg, 60.29%) as a light yellow solid. LCMS (ESI) m/z: [M+H]=592.

### Step 2: Preparation of N-(6-((2-(2,6-dioxopiperidin-3-yl)-1,3-dioxoisoindolin-5-yl)amino)hexyl)azetidine-3-sulfonamide (i73-3)

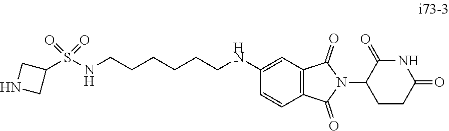

To a stirred mixture of tert-butyl 3-[(6-[[2-(2,6-dioxopiperidin-3-yl)-1,3-dioxoisoindol-5-yl]amino]hexyl)sulfamoyl]azetidine-1-carboxylate (61.8 mg, 0.104 mmol, 1.00 equiv) in DCM (2.00 mL) was added TEA (0.40 mL, 5.385 mmol, 51.56 equiv). After stirring for 1 hour at room temperature, the resulting mixture was concentrated under reduced pressure. The residue was used in the next step directly without further purification. LCMS (ESI) m/z: [M+H]⁺=492.

### Step 3: Preparation of 1-[[2,6-dimethoxy-4-(2-methyl-1-oxo-1,2-dihydro-2,7-naphthyridin-4-yl)phenyl]methyl]-N-(6-[[2-(2,6-dioxopiperidin-3-yl)-1,3-dioxo-2,3-dihydro-1H-isoindol-5-yl]amino]hexyl)azetidine-3-sulfonamide formic acid (compound D67 formic acid)

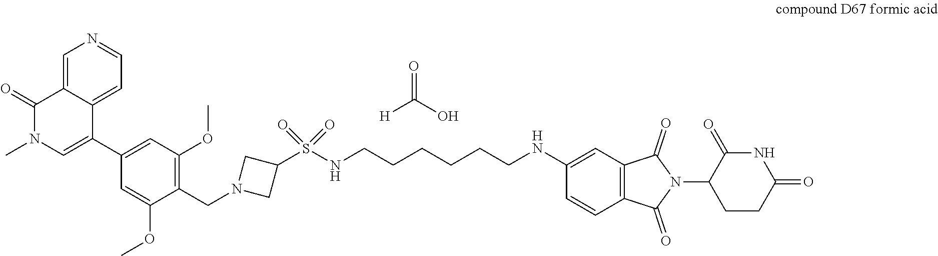

A mixture of N-(6-[[2-(2,6-dioxopiperidin-3-yl)-1,3-dioxo-2,3-dihydro-1H-isoindol-5-yl]amino]hexyl)azetidine-3-sulfonamide(51.36 mg, 0.104 mmol, 1.00 equiv) and 2,6-dimethoxy-4-(2-methyl-1-oxo-1,2-dihydro-2,7-naphthyridin-4-yl)benzaldehyde (33.89 mg, 0.104 mmol, 1.00 equiv) in DMF (2 mL) was stirred at room temperature. The reaction mixture was then adjusted to pH 8-9 with TEA. To the above mixture was added NaBH₃CN (19.70 mg, 0.313 mmol, 3.00 equiv) in portions, and the resulting mixture was stirred for 2 hours at room temperature. The resulting mixture was concentrated under reduced pressure, the residue was purified by Prep-HPLC (conditions: X Select CSH Prep C18 OBD Column, 5 μm, 19*150 mm; mobile phase, Water (0.1% FA) and ACN (15% Phase B up to 30% in 14 minutes); Detector, UV). This gave 1-[[2,6-dimethoxy-4-(2-methyl-1-oxo-1,2-dihydro-2,7-naphthyridin-4-yl)phenyl]methyl]-N-(6-[[2-(2,6-dioxopiperidin-3-yl)-1,3-dioxo-2,3-dihydro-1H-isoindol-5-yl]amino]hexyl)azetidine-3-sulfonamide formic acid (13 mg, 14.12%) as a yellow solid. ¹H NMR (400 MHz, DMSO-d6) δ 9.45 (s, 1H), 8.73 (d, J=5.7 Hz, 1H), 8.14 (s, 0.2H, FA), 7.87 (s, 1H), 7.56 (d, J=5.7 Hz, 1H), 7.51 (d, J=8.3 Hz, 1H), 7.27 (br s, 1H), 6.94 (d, J=2.0 Hz, 1H), 6.82 (dd, J=8.2, 2.0 Hz, 1H), 6.78 (s, 2H), 6.56 (d, J=8.2 Hz, 2H), 5.10 (dd, J=13.0, 5.4 Hz, 1H), 4.01 (br s, 2H), 3.84 (s, 7H), 3.60 (s, 6H), 3.47-3.35 (m, 2H), 3.05-2.83 (m, 3H), 2.77-2.65 (m, 1H), 2.49-2.41 (m, 2H), 2.03-1.96 (m, 1H), 1.39 (t, J=7.0 Hz, 4H), 1.24 (s, 4H). LCMS (ESI) m/z: [M+H]⁺=800.25.

### Example 74—Preparation of N-[3-[([[2,6-dimethoxy-4-(2-methyl-1-oxo-2,7-naphthyridin-4-yl)phenyl]methyl](meth-yl)amino)methyl]bicyclo[1.1.1]pentan-1-yl]-3-[2-(2-[[2-(2,6-dioxopiperidin-3-yl)-1,3-dioxoisoindol-4-yl]amino]ethoxy)ethoxy]propanamide formic acid (compound D68 formic acid)

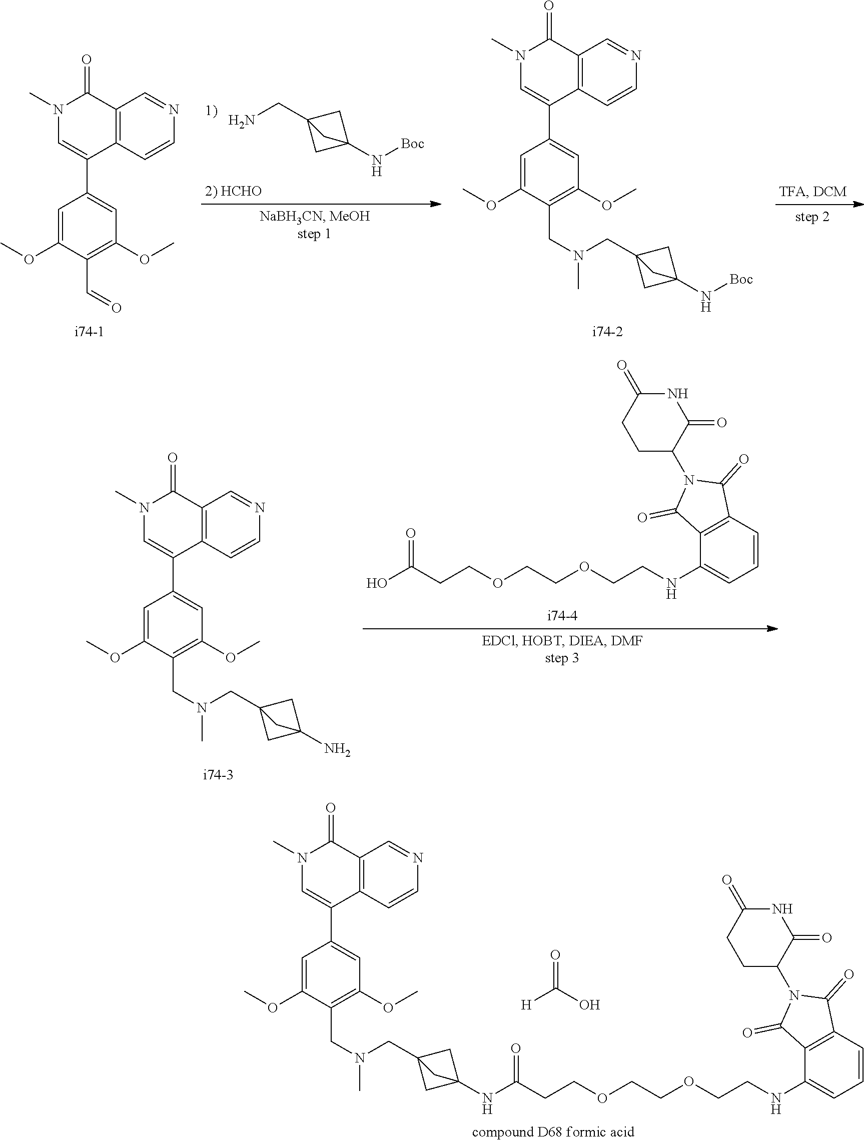

### Step 1: Preparation of tert-butyl N-[3-[([[2,6-dimethoxy-4-(2-methyl-1-oxo-2,7-naphthyridin-4-yl)phenyl]methyl]amino)methyl]bicyclo[1.1.1]pentan-1-yl]carbamate (i74-2)

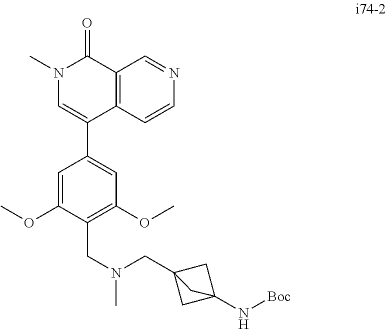 [SMILES: COc1cc(-c2cn(C)c(=O)c3cnccc23)cc(OC)c1CN(C)CC12CC(NC(=O)OC(C)(C)C)(C1)C2]

To a stirred mixture of 2,6-dimethoxy-4-(2-methyl-1-oxo-2,7-naphthyridin-4-yl)benzaldehyde (200.00 mg, 0.617 mmol, 1.00 equiv) and tert-butyl N-[3-(aminomethyl)bicyclo[1.1.1]pentan-1-yl]carbamate (i44.00 mg, 0.678 mmol, 1.10 equiv) in MeOH (1 mL) was added NaBH₃CN (77.50 mg, 1.233 mmol, 2.00 equiv) in portions at room temperature. The resulting mixture was stirred for 2 hours at room temperature. To the above mixture was added formaldehyde (0.50 mL). The resulting mixture was stirred for 1 hour at room temperature. The reaction mixture was filtered, and the filtrate was concentrated under reduced pressure. The crude product was purified by flash silica chromatography, elution gradient 0 to 30% EtOAc in petroleum ether. Pure fractions was concentrated under vacuum to afford tert-butyl N-[3-[([[2,6-dimethoxy-4-(2-methyl-1-oxo-2,7-naphthyridin-4-yl)phenyl]methyl]amino)methyl]bicyclo[1.1.1]pentan-1-yl]carbamate (284.8 mg) as a yellow gum. LCMS (ESI) m/z: [M+H]⁺=535.

### Step 2: Preparation of 4-(4-[[([3-aminobicyclo[1.1.1]pentan-1-yl]methyl)(methyl)amino]methyl]-3,5-dimeth oxyphenyl)-2-methyl-2,7-naphthyridin-1-one (i74-3)

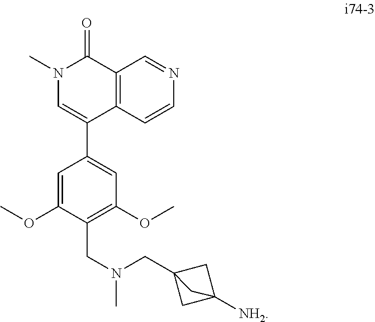 [SMILES: COc1cc(-c2cn(C)c(=O)c3cnccc23)cc(OC)c1CN(C)CC12CC(N)(C1)C2]

A mixture of tert-butyl N-[3-[([[2,6-dimethoxy-4-(2-methyl-1-oxo-2,7-naphthyridin-4-yl)phenyl]methyl](me-thyl)amino)methyl]bicyclo[1.1.1]pentan-1-yl]carbamate (284.80 mg) and TFA (1.00 mL) in DCM (1 mL) was stirred for overnight at room temperature. The reaction mixture was concentrated under vacuum to afford 4-(4-[[([3-aminobicyclo[1.1.1]pentan-1-yl]methyl)(methyl)amino]methyl]-3,5-dimethoxyphenyl)-2-me thyl-2,7-naphthyridin-1-one (639.4 mg, crude) as a yellow gum. LCMS (ESI) m/z: [M+H]⁺=435.

### Step 3: Preparation of N-[3-[([[2,6-dimethoxy-4-(2-methyl-1-oxo-2,7-naphthyridin-4-yl)phenyl]methyl](met-hyl)amino)methyl]bicyclo[1.1.1]pentan-1-yl]-3-[2-(2-[[2-(2,6-dioxopiperidin-3-yl)-1,3-dioxoisoindol-4-yl]ami-no]ethoxy)ethoxy]propanamide formic acid (compound D68 formic acid)

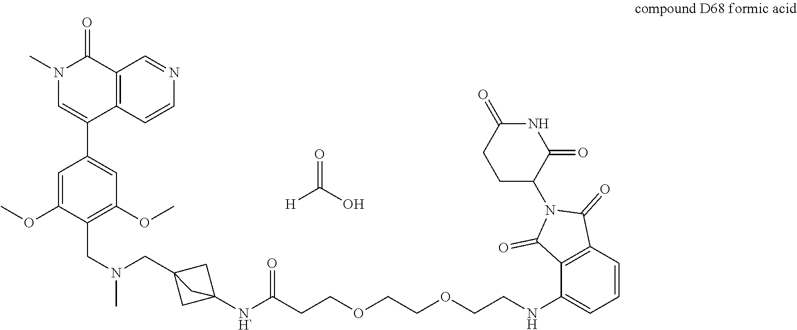

To a stirred solution of 4-(4-[[([3-aminobicyclo[1.1.1]pentan-1-yl]methyl)(methyl)amino]methyl]-3,5-dimet-hoxyphenyl)-2-methyl-2,7-naphthyridin-1-one (20.05 mg, 0.046 mmol, 1 equiv) and 3-[2-(2-[[2-(2,6-diox-opiperidin-3-yl)-1,3-dioxoisoindol-4-yl]amino]ethoxy)ethoxy]propanoic acid (20.00 mg, 0.046 mmol, 1.00 equiv) in DMF (1 mL) was added EDCl (17.69 mg, 0.092 mmol, 2.00 equiv), HOBT (12.47 mg, 0.092 mmol, 2.00 equiv), and DIEA (23.86 mg, 0.185 mmol, 4.00 equiv). The resulting mixture was stirred overnight at room temperature. Without any additional work-up, the mixture was purified by prep-HPLC (conditions: Column: Gemini-NX C18 AXAI Packed, 21.2*150 mm 5 μm; Mobile Phase A: Water (0.1% FA), Mobile Phase B:ACN; Flow rate:25 mL/minute; Gradient:8 B to 25 B in 12 minutes; 254/220 nm; R_{T}: 11.04 minutes) to afford N-[3-[([[2,6-dimethoxy-4-(2-methyl-1-oxo-2,7-naphthyridin-4-yl)phenyl]methyl](methy-1)amino)methyl]bicycle[1.1.1]pentan-1-yl]-3-[2-(2-[[2-(2,6-dioxopiperidin-3-yl)-1,3-dioxoisoindol-4-yl]amin-o]ethoxy)ethoxy]propanamide (3.4 mg, 8.67%) as a yellow solid. ¹H NMR (300 MHz, DMSO-d6) δ 11.09 (s, 1H), 9.45 (s, 1H), 8.72 (d, J=5.6 Hz, 1H), 8.29 (s, 1H), 8.23 (brs, 1.0H, FA), 7.87 (s, 1H), 7.58 (t, J=7.6 Hz, 2H), 7.14 (d, J=8.6 Hz, 1H), 7.03 (d, J=7.1 Hz, 1H), 6.72 (s, 2H), 6.61 (t, J=5.8 Hz, 1H), 5.06 (dd, J=12.7, 5.4 Hz, 1H), 3.81 (s, 6H), 3.58-3.54 (m, 5H), 3.54-3.49 (m, 6H), 3.48-3.42 (m, 4H), 2.96-2.81 (m, 1H), 2.64-2.58 (m, 1H), 2.55 (s, 3H), 2.26 (t, J=6.4 Hz, 2H), 2.12 (s, 3H), 2.08-1.98 (m, 1H), 1.92 (s, 6H). LCMS (ESI) m/z: [M+H]⁺=850.50.

### Example 75—Preparation of N-[3-[([[2,6-dimethoxy-4-(2-methyl-1-oxo-2,7-naphthyridin-4-yl)phenyl]methyl](meth-yl)amino)methyl]bicyclo[1.1.1]pentan-1-yl]-5-[[2-(2,6-dioxopiperidin-3-yl)-1,3-dioxoisoindol-4-yl]ox-y]pentanamide (compound D69)

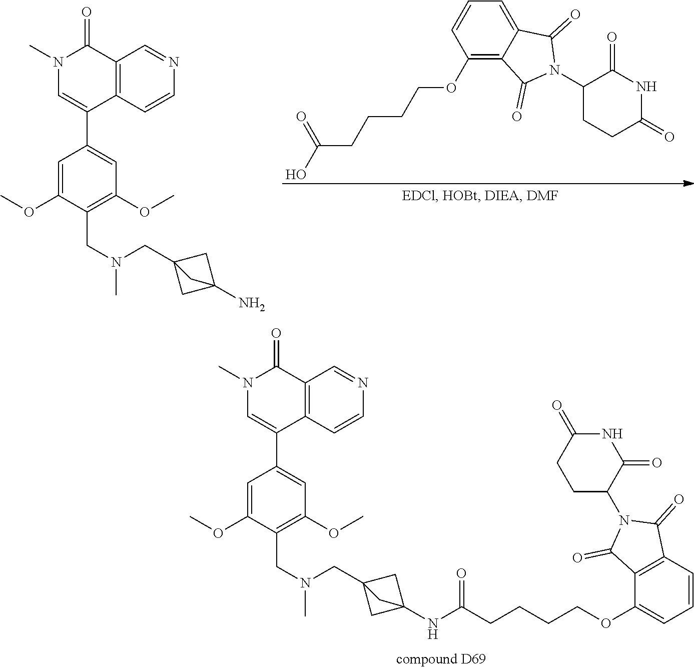

To a stirred solution of 4-(4-[[([3-aminobicyclo[1.1.1]pentan-1-yl]methyl)(methyl)amino]methyl]-3,5-dimethoxyphenyl)-2-methyl-2,7-naphthyridin-1-one (23.22 mg, 0.053 mmol, 1.00 equiv) and 5-[[2-(2,6-dioxopiperidin-3-yl)-1,3-dioxoisoindol-4-yl]oxy]pentanoic acid (20.00 mg, 0.053 mmol, 1.00 equiv) in DMF (1 mL) was added EDCl (20.48 mg, 0.107 mmol, 2.00 equiv) and HOBT (14.44 mg, 0.107 mmol, 2.00 equiv) at room temperature. To the above mixture was added DIEA (27.62 mg, 0.214 mmol, 4.00 equiv). The resulting mixture was stirred for overnight at room temperature. Without any additional work-up, the mixture was purified by prep-HPLC (conditions: SunFire Prep C18 OBD Column, 19×150 mm 5 μm 10 nm; Mobile Phase A: Water (0.1% FA), Mobile Phase B:ACN; Flow rate:25 mL/minute; Gradient:13 B to 22 B in 13 minutes; 254/220 nm; R_{T}: 12.5 minutes) to afford N-[3-[([[2,6-dimethoxy-4-(2-methyl-1-oxo-2,7-naphthyridin-4-yl)phenyl]methyl](methyl)amino)methy-1]bicyclo[1.1.1]pentan-1-yl]-5-[[2-(2,6-dioxopiperidin-3-yl)-1,3-dioxoisoindol-4-yl]oxy]pentanamide (6.9 mg, 17.75%) as a white solid. ¹H NMR (300 MHz, DMSO-d6) δ 11.10 (s, 1H), 9.46 (d, J=0.8 Hz, 1H), 8.74 (d, J=5.7 Hz, 1H), 8.36 (s, 1H), 7.88 (s, 1H), 7.82 (dd, J=8.5, 7.2 Hz, 1H), 7.60-7.42 (m, 3H), 6.79 (s, 2H), 5.08 (dd, J=12.8, 5.4 Hz, 1H), 4.21 (t, J=6.0 Hz, 2H), 3.86 (s, 6H), 3.61 (s, 3H), 3.40 (s, 2H), 2.98-2.80 (m, 2H), 2.62 (s, 2H), 2.46-2.30 (m, 4H), 2.15-2.00 (m, 9H), 1.78-1.64 (m, 4H). LCMS (ESI) m/z: [M+H]⁺=791.40.

### Example 76—Preparation of 5-(4-[2-[3-([[2,6-dimethoxy-4-(2-methyl-1-oxo-2,7-naphthyridin-4-yl)phenyl]meth yl](methyl)amino)propoxy]ethyl]piperazin-1-yl)-2-(2,6-dioxopiperidin-3-yl)isoindole-1,3-dione formic acid (compound D70 formic acid)

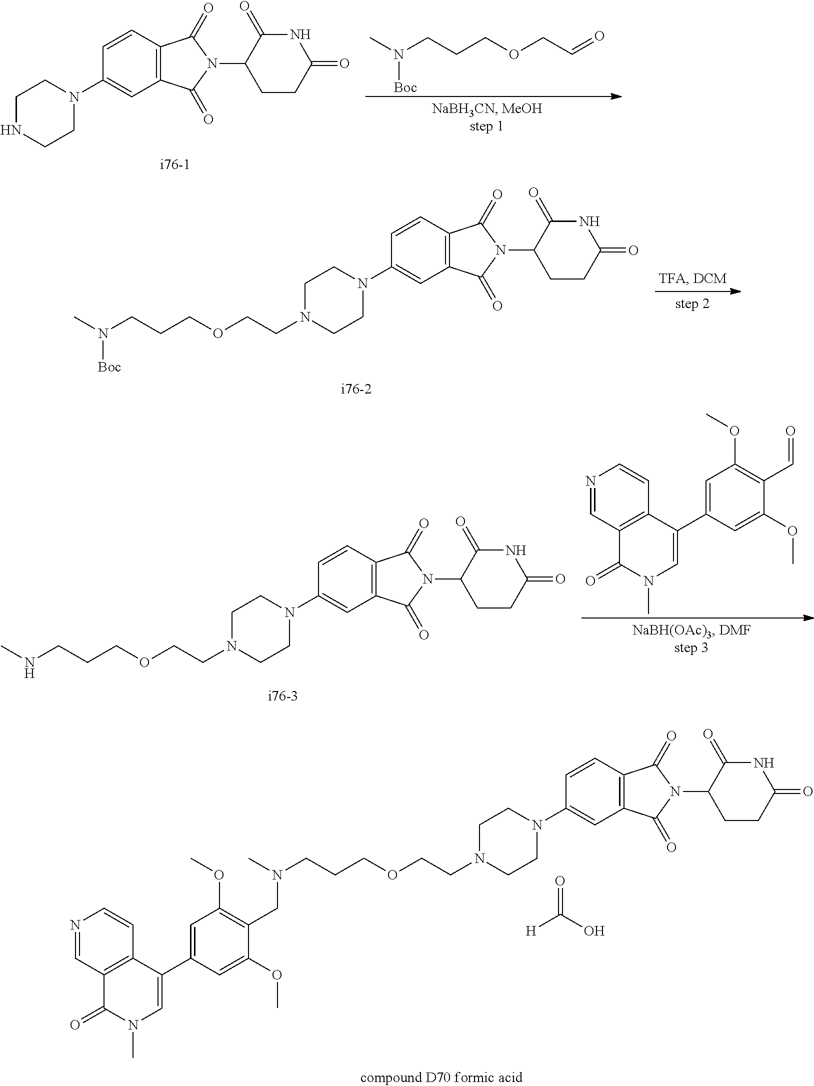

### Step 1: Preparation of tert-butyl N-[3-(2-[4-[2-(2,6-dioxopiperidin-3-yl)-1,3-dioxoisoindol-5-yl]piperazin-1-yl]ethoxy)propyl]-N-methylcarbamate (i76-2)

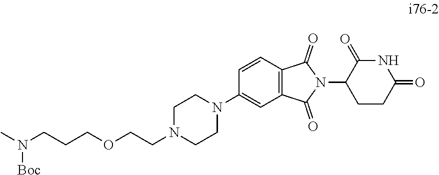 [SMILES: CN(CCCOCCN1CCN(c2ccc3c(c2)C(=O)N(C2CCC(=O)NC2=O)C3=O)CC1)C(=O)OC(C)(C)C]

To a stirred solution of 2-(2,6-dioxopiperidin-3-yl)-5-(piperazin-1-yl)isoindole-1,3-dione (250.00 mg, 0.730 mmol, 1.00 equiv) and tert-butyl N-methyl-N-[3-(2-oxoethoxy)propyl]carbamate (168.90 mg, 0.730 mmol, 1 equiv) in MeOH (3.00 mL) was added NaBH₃CN (91.78 mg, 1.460 mmol, 2 equiv). The mixture was stirred at room temperature for 2 hours. The resulting mixture was concentrated under reduced pressure. The residue was purified by Prep-TLC (Petroleum ether/EtOAc 1:3) to afford tert-butyl N-[3-(2-[4-[2-(2,6-dioxopiperidin-3-yl)-1,3-dioxoisoindol-5-yl]piperazin-1-yl]ethoxy)propyl]-N-methylcarbamate (400 mg, crude) as a dark grey solid. LCMS (ESI) m/z: [M+H]+=558.

### Step 2: Preparation of 2-(2,6-dioxopiperidin-3-yl)-5-(4-[2-[3-(methylamino)propoxy]ethyl]piperazin-1-yl)isoindole-1,3-dione (i76-3)

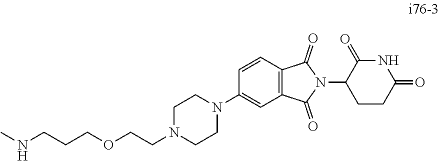 [SMILES: CNCCCOCCN1CCN(c2ccc3c(c2)C(=O)N(C2CCC(=O)NC2=O)C3=O)CC1]

To a stirred solution of tert-butyl N-[3-(2-[4-[2-(2,6-dioxopiperidin-3-yl)-1,3-dioxoisoindol-5-yl]piperazin-1-yl]ethoxy)propyl]-N-methylcarbamate (200.00 mg, 0.359 mmol, 1.00 equiv) in DCM (4.00 mL, 62.920 mmol) was added TFA (1.00 mg, 0.009 mmol). The mixture was stirred at room temperature for 2 hours. The resulting mixture was concentrated under reduced pressure to afford 2-(2,6-dioxopiperidin-3-yl)-5-(4-[2-[3-(methylamino)propoxy]ethyl]piperazin-1-yl)isoindole-1,3-dione (280 mg, crude) as a dark grey solid. LCMS (ESI) m/z: [M+H]+=458.

### Step 3: Preparation of 5-(4-[2-[3-([[2,6-dimethoxy-4-(2-methyl-1-oxo-2,7-naphthyridin-4-yl)phenyl]methyl](methyl)amino)propoxy]ethyl]piperazin-1-yl)-2-(2,6-dioxopiperidin-3-yl)isoindole-1,3-dione formic acid (compound D70 formic acid)

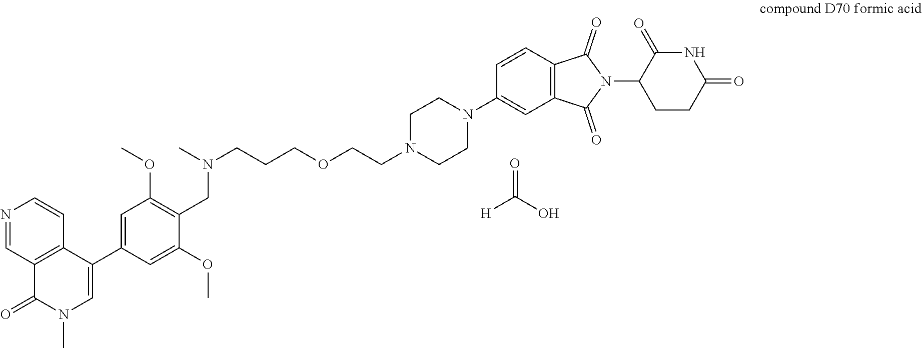 [SMILES: COc1cc(-c2cn(C)c(=O)c3cnccc23)cc(OC)c1CN(C)CCCOCCN1CCN(c2ccc3c(c2)C(=O)N(C2CCC(=O)NC2=O)C3=O)CC1 ; O=CO]

To a stirred solution of 2-(2,6-dioxopiperidin-3-yl)-5-(4-[2-[3-(methylamino)propoxy]ethyl]piperazin-1-yl)isoindole-1,3-dione (100.00 mg, 0.219 mmol, 1.00 equiv) and 2,6-dimethoxy-4-(2-methyl-1-oxo-2,7-naphthyridin-4-yl)benzaldehyde (70.89 mg, 0.219 mmol, 1 equiv) in DMF (1.50 mL) was added NaBH(OAc)₃ (92.65 mg, 0.437 mmol, 2 equiv). The mixture was stirred at room temperature for 2 hours. The crude product (100 mg) was purified by Prep-HPLC (conditions: SunFire C18 OBD Prep Column, 100 Å, 5 μm, 19 mm×250 mm; Mobile Phase A: Water (0.1% FA), Mobile Phase B:ACN; Flow rate:25 mL/minute; Gradient:5 B to 13 B in 15 minutes; 254 nm; RT: 12.23 minutes) to afford 5-(4-[2-[3-([[2,6-dimethoxy-4-(2-methyl-1-oxo-2,7-naphthyridin-4-yl)phenyl]methyl](methyl)amino)propoxy]ethyl]piperazin-1-yl)-2-(2,6-dioxopiperidin-3-yl)isoindole-1,3-dione; formic acid(10 mg, 5.38%) as a yellow solid. ¹H NMR (300 MHz, Methanol-d4) δ 9.51 (d, J=18.3 Hz, 1H), 8.68 (d, J=5.7 Hz, 1H), 8.53 (brs, 4.1H, FA), 7.76 (s, 1H), 7.64 (d, J=7.4 Hz, 2H), 7.25 (s, 1H), 7.17 (d, J=8.6 Hz, 1H), 6.90 (s, 2H), 5.11-5.04 (m, 2H), 4.69-4.53 (m, 2H), 4.47 (s, 2H), 4.00 (s, 6H), 3.74-3.62 (m, 7H), 3.40 (d, J=5.5 Hz, 4H), 2.91 (s, 3H), 2.87-2.73 (m, 3H), 2.69 (s, 6H), 2.23-2.08 (m, 3H). LCMS (ESI) m/z: [M+H]⁺=766.45.

### Example 77—Preparation of 4-[2-([[2,6-dimethoxy-4-(2-methyl-1-oxo-2,7-naphthyridin-4-yl)phenyl]methyl](meth yl)amino)acetamido]-N-(3-[[2-(2,6-dioxopiperidin-3-yl)-1,3-dioxoisoindol-4-yl]amino]bicycle[1.1.1]pentan-1-yl)butanamide (compound D71)

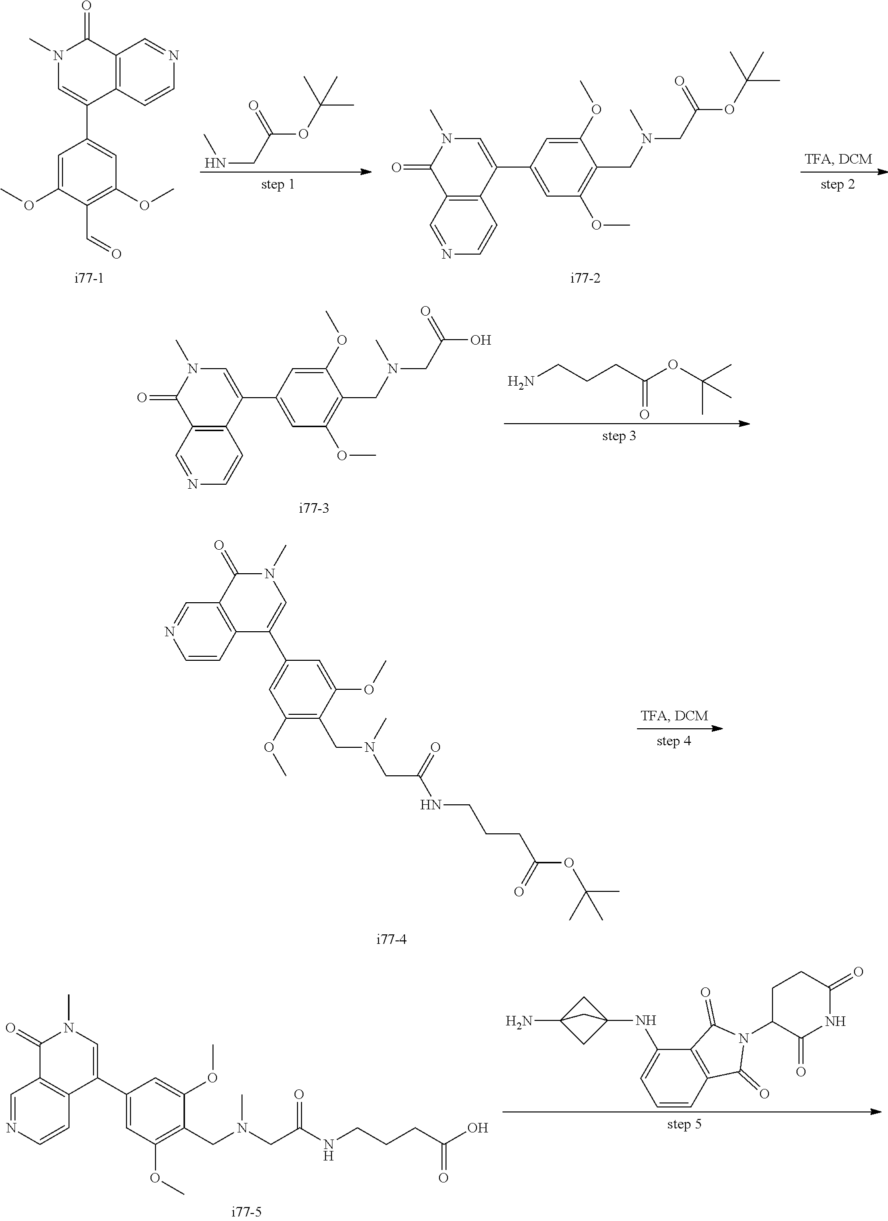

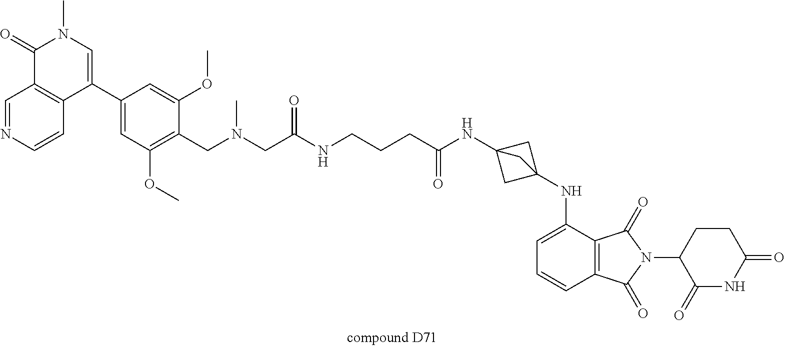 [SMILES: COc1cc(-c2cn(C)c(=O)c3cnccc23)cc(OC)c1CN(C)CC(=O)NCCCC(=O)NC12CC(Nc3cccc4c3C(=O)N(C3CCC(=O)NC3=O)C4=O)(C1)C2]

### Step 1: Preparation of tert-butyl N-(2,6-dimethoxy-4-(2-methyl-1-oxo-1,2-dihydro-2,7-naphthyridin-4-yl) benzyl)-N-methylglycinate (i77-2)

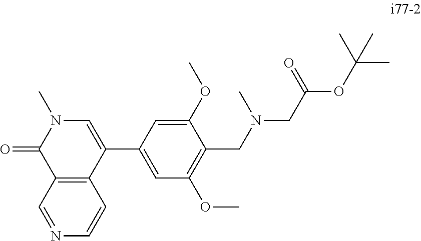 [SMILES: COc1cc(-c2cn(C)c(=O)c3cnccc23)cc(OC)c1CN(C)CC(=O)OC(C)(C)C]

To a stirred solution of 2,6-dimethoxy-4-(2-methyl-1-oxo-2,7-naphthyridin-4-yl)benzaldehyde (250.00 mg, 0.771 mmol, 1.00 equiv) and tert-butyl 2-(methylamino)acetate (111.92 mg, 0.771 mmol, 1.00 equiv) in MeOH (10.00 mL) was added NaBH₃CN (96.88 mg, 1.542 mmol, 2.00 equiv) in portions at 50° C. under nitrogen atmosphere. The mixture was allowed to cool down to room temperature. The reaction was quenched with Water at room temperature. The aqueous layer was extracted with EtOAc (3×30 mL). The resulting solid was dried under vacuum. The residue was purified by reverse flash chromatography (conditions: column, C18 silica gel; mobile phase, MeOH in water, 10% to 50% gradient in 10 minutes; detector, UV 254 nm). This resulted in tert-butyl N-(2,6-dimethoxy-4-(2-methyl-1-oxo-1,2-dihydro-2,7-naphthyridin-4-yl)benzyl)-N-methylglycinate (101 mg, 28.92%) as a yellow oil. LCMS (ESI) m/z: [M+H]+=454.

### Step 2: Preparation of N-(2,6-dimethoxy-4-(2-methyl-1-oxo-1,2-dihydro-2,7-naphthyridin-4-yl)benzyl)-N-methylglycine (i77-3)

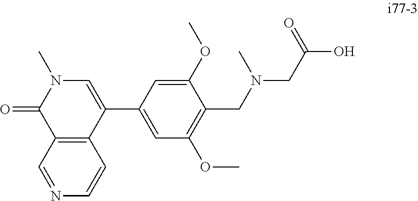 [SMILES: COc1cc(-c2cn(C)c(=O)c3cnccc23)cc(OC)c1CN(C)CC(=O)O]

A solution of tert-butyl 2-([[2,6-dimethoxy-4-(2-methyl-1-oxo-2,7-naphthyridin-4-yl)phenyl]methyl](methyl) amino) acetate (101.00 mg, 0.223 mmol, 1.00 equiv) and TFA (7.21 mL, 63.270 mmol, 436.14 equiv) in DCM (29.00 mL) was stirred for 15 hours at room temperature under nitrogen atmosphere. The resulting mixture was concentrated under vacuum. The residue (108 mg, crude) was used in the next step directly without further purification. LCMS (ESI) m/z: [M+H]+=398.

### Step 3: Preparation of tert-butyl 4-(2-((2,6-dimethoxy-4-(2-methyl-1-oxo-1,2-dihydro-2,7-naphthyridin-4-yl)benzyl)(methyl)amino)acetamido)butanoate (i77-4)

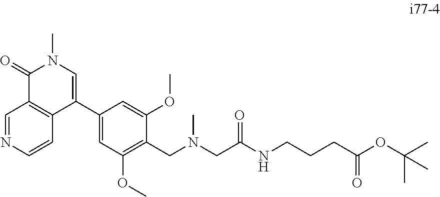 [SMILES: COc1cc(-c2cn(C)c(=O)c3cnccc23)cc(OC)c1CN(C)CC(=O)NCCCC(=O)OC(C)(C)C]

A solution of ([[2,6-dimethoxy-4-(2-methyl-1-oxo-2,7-naphthyridin-4-yl)phenyl]methyl](methyl)amino)acetic acid (108 mg (crude), 0.272 mmol, 1.00 equiv), DIEA (105.36 mg, 0.815 mmol, 3.00 equiv), and HATU (206.53 mg, 0.543 mmol, 2.00 equiv) in DMF (2.00 mL) was stirred for 30 minutes at room temperature under nitrogen atmosphere. To the above mixture was added tert-butyl 4-aminobutanoate (43.27 mg, 0.272 mmol, 1.00 equiv) at room temperature. The resulting mixture was stirred for additional 12 hours at room temperature. The residue was purified by reverse flash chromatography (conditions: column, C18 silica gel; mobile phase, MeOH in water, 10% to 50% gradient in 10 minutes; detector, UV 254 nm). This resulted in tert-butyl 4-(2-((2,6-dimethoxy-4-(2-methyl-1-oxo-1,2-dihydro-2,7-naphthyridin-4-yl)benzyl)(methyl)amino)acetamido) butanoate (75 mg, 62.33%) as a yellow oil. LCMS (ESI) m/z: [M+H]+=539.

### Step 4: Preparation of 4-(2-((2,6-dimethoxy-4-(2-methyl-1-oxo-1,2-dihydro-2,7-naphthyridin-4-yl)benzyl)(methyl)amino)acetamido)butanoicacid (i77-5)

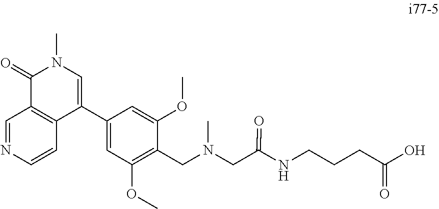 [SMILES: COc1cc(-c2cn(C)c(=O)c3cnccc23)cc(OC)c1CN(C)CC(=O)NCCCC(=O)O]

A solution of tert-butyl 4-[2-([[2,6-dimethoxy-4-(2-methyl-1-oxo-2,7-naphthyridin-4-yl)phenyl]methyl](meth yl)amino) acetamido]butanoate (75.00 mg, 0.139 mmol, 1.00 equiv) and TFA (1 mL) in DCM (4.00 mL) was stirred for 2 hours at room temperature under nitrogen atmosphere. The resulting mixture was concentrated under vacuum. The residue (73 mg, crude) was used in the next step directly without further purification. LCMS (ESI) m/z: [M+H]+=483.

### Step 5: Preparation of 4-(2-((2,6-dimethoxy-4-(2-methyl-1-oxo-1,2-dihydro-2,7-naphthyridin-4-yl)benzyl) (methyl)amino)acetamido)-N-(3-((2-(2,6-dioxopiperidin-3-yl)-1,3-dioxoisoindolin-4-yl)amino)bicycle[1.1.1]pentan-1-yl)butanamide (compound D71)

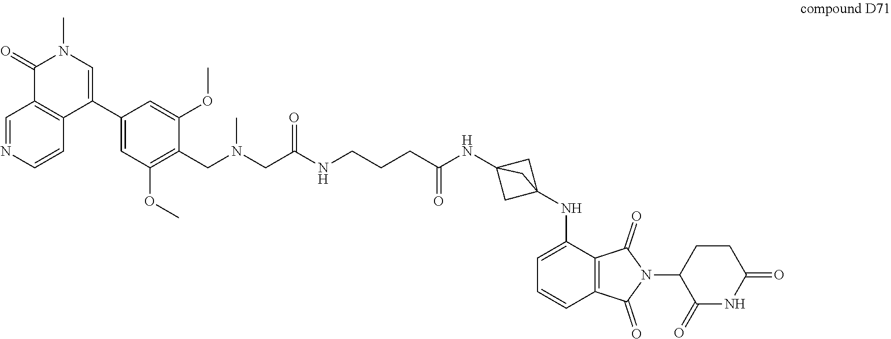 [SMILES: COc1cc(-c2cn(C)c(=O)c3cnccc23)cc(OC)c1CN(C)CC(=O)NCCCC(=O)NC12CC(Nc3cccc4c3C(=O)N(C3CCC(=O)NC3=O)C4=O)(C1)C2]

To a stirred solution of 4-[2-([[2,6-dimethoxy-4-(2-methyl-1-oxo-2,7-naphthyridin-4-yl)phenyl]methyl](methyl)amino) acetamido]butanoic acid (73.00 mg(crude), 0.151 mmol, 1.00 equiv), DIEA (58.66 mg, 0.454 mmol, 3.00 equiv), and EDCl (58.00 mg, 0.303 mmol, 2.00 equiv) in DMF (2.00 mL) was added HOBT (40.88 mg, 0.303 mmol, 2.00 equiv) in portions at room temperature under nitrogen atmosphere. The reaction mixture was irradiated with microwave radiation for 1 hour at room temperature. To the above mixture was added 4-([3-aminobicyclo[1.1.1]pentan-1-yl]amino)-2-(2,6-dioxopiperidin-3-yl) isoindole-1,3-dione (53.61 mg, 0.151 mmol, 1.00 equiv) at room temperature. The resulting mixture was stirred for additional 2 days at room temperature. The residue was purified by reverse flash chromatography (conditions: column, C18 silica gel; mobile phase, MeOH in water, 10% to 50% gradient in 10 minutes; detector, UV 254 nm). The crude product (70 mg) was purified by Prep-HPLC (conditions: Atlantis HILIC OBD Column 19*150 mm, 5 μm; Mobile Phase A: Water (0.1% FA), Mobile Phase B: ACN; Flow rate: 40 mL/minute; Gradient: 24% B to 24% B in 12 minutes; 254/220 nm; Rt: 11.43 minutes) to afford 4-[2-([[2,6-dimethoxy-4-(2-methyl-1-oxo-2,7-naphthyridin-4-yl)phenyl]methyl](methyl)amino) acetamido]-N-(3-[[2-(2,6-dioxopiperidin-3-yl)-1,3-dioxoisoindol-4-yl]amino]bicyclo[1.1.1]pentan-1-yl)butane mide (10 mg, 8.07%) as a light yellow solid. ¹H NMR (400 MHz, Methanol-d4) δ 9.56 (s, 1H), 8.70 (d, J=6.0 Hz, 1H), 7.88 (d, J=1.4 Hz, 1H), 7.75 (d, J=5.9 Hz, 1H), 7.58 (dd, J=9.5, 5.0 Hz, 1H), 7.27 (dd, J=8.6, 3.5 Hz, 1H), 7.14 (d, J=7.2 Hz, 1H), 6.89 (s, 2H), 5.08 (dd, J=12.4, 5.4 Hz, 1H), 4.56 (d, J=5.7 Hz, 2H), 4.01-3.97 (m, 7H), 3.93-3.87 (m, 1H), 3.73 (s, 3H), 3.29-3.23 (m, 2H), 2.97 (s, 3H), 2.90-2.83 (m, 1H), 2.80-2.68 (m, 2H), 2.43 (s, 6H), 2.22 (t, J=7.3 Hz, 2H), 2.16-2.10 (m, 1H), 1.80 (p, J=7.2 Hz, 2H). LCMS (ESI) m/z: [M+H]⁺=819.35.

### Example 78—Preparation of N-[[2,6-dimethoxy-4-(2-methyl-1-oxo-2,7-naphthyridin-4-yl)phenyl]methyl]-5-[9-[2-(2,6-dioxopiperidin-3-yl)-1,3-dioxoisoindol-5-yl]-1-oxa-4,9-diazaspiro[5.5]undecan-4-yl]-N-methyl pentanamide formic acid (compound D72 formic acid)

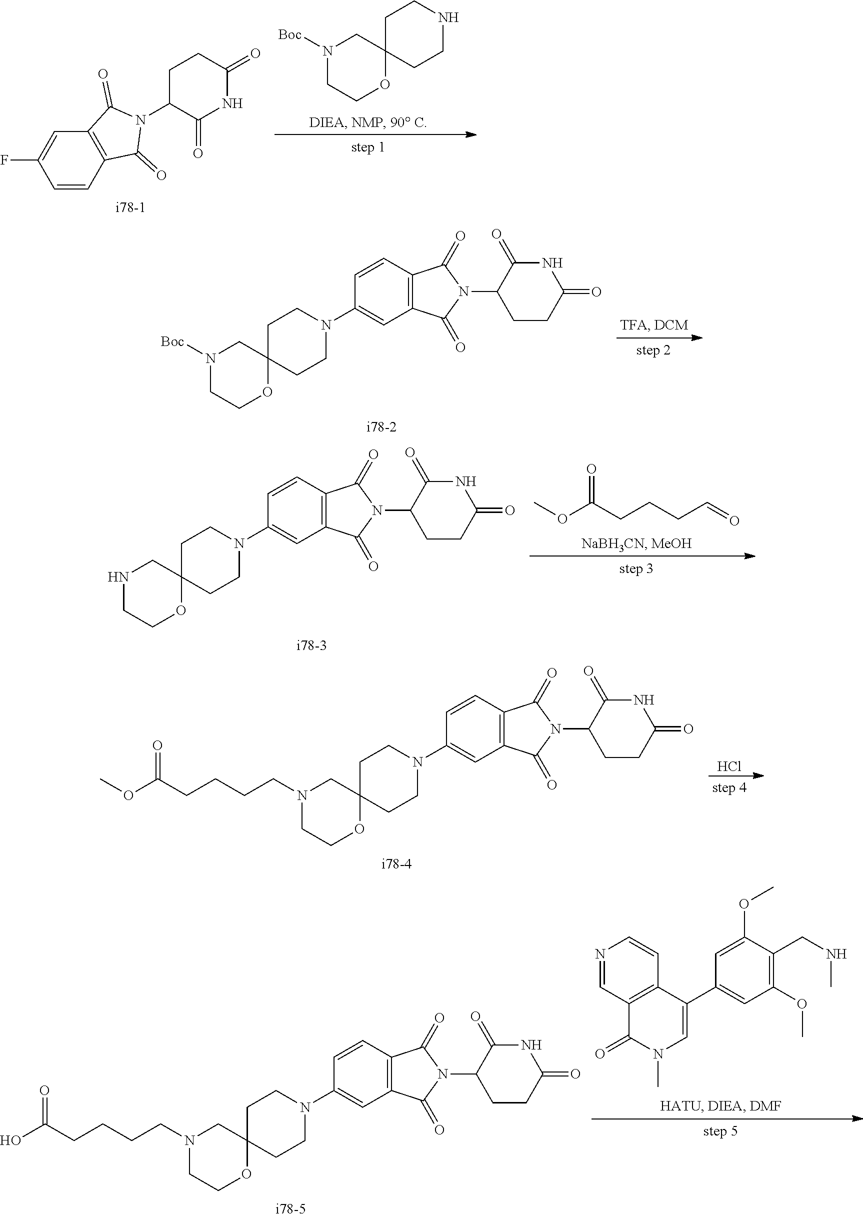

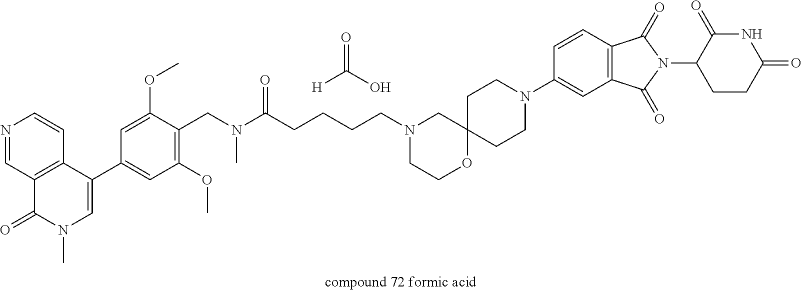

### Step 1: Preparation of 2-(2,6-dioxopiperidin-3-yl)-5-[1-oxa-4,9-diazaspiro[5.5]undecan-9-yl]isoindole-1,3-dione (i78-2)

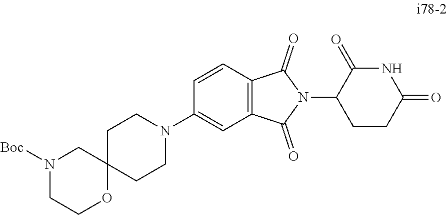 [SMILES: CC(C)(C)OC(=O)N1CCOC2(CCN(c3ccc4c(c3)C(=O)N(C3CCC(=O)NC3=O)C4=O)CC2)C1]

To a stirred solution of 2-(2,6-dioxopiperidin-3-yl)-5-fluoroisoindole-1,3-dione (1.50 g, 5.430 mmol, 1.00 equiv) and tert-butyl 1-oxa-4,9-diazaspiro[5.5]undecane-4-carboxylate (1.67 g, 6.516 mmol, 1.20 equiv) in NMP (10.00 mL) was added DIEA (1.40 g, 10.861 mmol, 2.00 equiv) dropwise at room temperature. The resulting mixture was stirred for 6 hours at 90° C. under nitrogen atmosphere. The residue was purified by reverse flash chromatography (conditions: column, C18 silica gel; mobile phase, ACN in water, 10% to 50% gradient in 20 minutes; detector, UV 254 nm). This resulted in tert-butyl 9-(2-(2,6-dioxopiperidin-3-yl)-1,3-dioxoisoindolin-5-yl)-1-oxa-4,9-diazaspiro[5.5]undecane-4-carboxylate (2 g, 72%) as a green oil. LCMS (ESI) m/z: [M+H]+=513.

### Step 2: Preparation of 2-(2,6-dioxopiperidin-3-yl)-5-[1-oxa-4,9-diazaspiro[5.5]undecan-9-yl]isoindole-1,3-dione (i78-3)

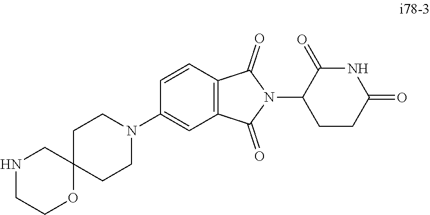 [SMILES: O=C1CCC(N2C(=O)c3ccc(N4CCC5(CC4)CNCCO5)cc3C2=O)C(=O)N1]

To a stirred solution of tert-butyl 9-[2-(2,6-dioxopiperidin-3-yl)-1,3-dioxoisoindol-5-yl]-1-oxa-4,9-diazaspiro[5.5]undecane-4-carboxylate (430.00 mg, 0.839 mmol, 1.00 equiv) in DCM (3.50 mL) was added TFA (1.00 mL). The mixture was stirred at room temperature for 1 hour. The resulting mixture was concentrated under reduced pressure to afford 2-(2,6-dioxopiperidin-3-yl)-5-[1-oxa-4,9-diazaspiro [5.5]undecan-9-yl]isoindole-1,3-dione (670 mg, crude) as a yellow solid. LCMS (ESI) m/z: [M+H]+=413

### Step 3: Preparation of methyl 5-[9-[2-(2,6-dioxopiperidin-3-yl)-1,3-dioxoisoindol-5-yl]-1-oxa-4,9-diazaspiro[5.5]undecan-4-yl]pentanoate (i78-4)

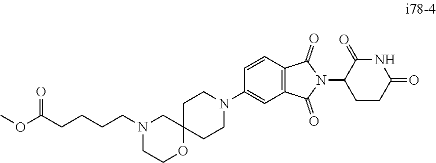 [SMILES: COC(=O)CCCCN1CCOC2(CCN(c3ccc4c(c3)C(=O)N(C3CCC(=O)NC3=O)C4=O)CC2)C1]

To a stirred solution of 2-(2,6-dioxopiperidin-3-yl)-5-[1-oxa-4,9-diazaspiro[5.5]undecan-9-yl]isoindole-1,3-dione (200.00 mg, 0.485 mmol, 1.00 equiv) and methyl 5-oxopentanoate (75.73 mg, 0.582 mmol, 1.2 equiv) in MeOH (2.00 mL) was added NaBH₃CN (60.95 mg, 0.970 mmol, 2 equiv). The mixture was stirred at room temperature for 2 hours. The resulting mixture was concentrated under reduced pressure. The residue was purified by Prep-TLC (Petroleum ether/EtOAc 1:3) to afford methyl 5-[9-[2-(2,6-dioxopiperidin-3-yl)-1,3-dioxoisoindol-5-yl]-1-oxa-4,9-diazaspiro[5.5]undecan-4-yl]pentanoate (80 mg, 31.33%) as a yellow solid. LCMS (ESI) m/z: [M+H]⁺=527.

### Step 4: Preparation of 5-[9-[2-(2,6-dioxopiperidin-3-yl)-1,3-dioxoisoindol-5-yl]-1-oxa-4,9-diazaspiro [5.5]undecan-4-yl]pentanoic acid (i78-5)

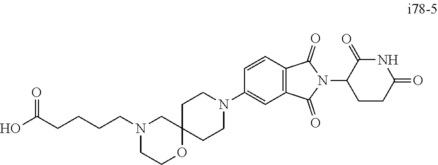 [SMILES: O=C(O)CCCCN1CCOC2(CCN(c3ccc4c(c3)C(=O)N(C3CCC(=O)NC3=O)C4=O)CC2)C1]

Methyl 5-[9-[2-(2,6-dioxopiperidin-3-yl)-1,3-dioxoisoindol-5-yl]-1-oxa-4,9-diazaspiro[5.5]undecan-4-yl]pentanoate (70.00 mg, 0.133 mmol, 1.00 equiv) was stirred at room temperature with HCl (aq.) for 2 hours. The resulting mixture was concentrated under reduced pressure. This resulted in 5-[9-[2-(2,6-dioxopiperidin-3-yl)-1,3-dioxoisoindol-5-yl]-1-oxa-4,9-diazaspiro[5.5]undecan-4-yl]pentanoic acid (70 mg, crude) as a yellow solid. LCMS (ESI) m/z: [M+H]⁺=513.

### Step 5: Preparation of N-[[2,6-dimethoxy-4-(2-methyl-1-oxo-2,7-naphthyridin-4-yl)phenyl]methyl]-5-[9-[2-(2,6-dioxopiperidin-3-yl)-1,3-dioxoisoindol-5-yl]-1-oxa-4,9-diazaspiro[5.5]undecan-4-yl]-N-methylpentanamide formic acid (compound D72 formic acid)

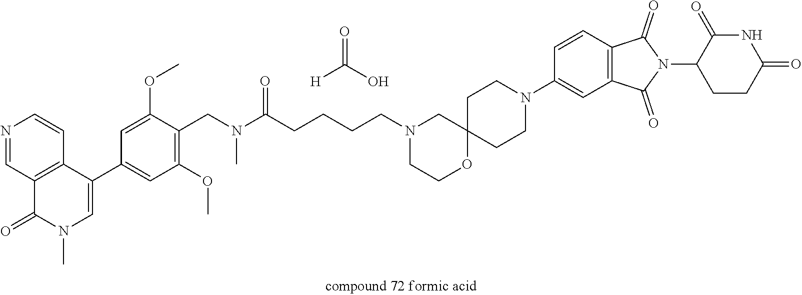

To a stirred solution of 5-[9-[2-(2,6-dioxopiperidin-3-yl)-1,3-dioxoisoindol-5-yl]-1-oxa-4,9-diazaspiro[5.5]undecan-4-yl]pentanoic acid (55.00 mg, 0.107 mmol, 1.00 equiv) and 4-[3,5-dimethoxy-4-[(methylamino)methyl]phenyl]-2-methyl-2,7-naphthyridin-1-one (36.42 mg, 0.107 mmol, 1.00 equiv) in DMF (1.00 mL) was added DIEA (69.34 mg, 0.537 mmol, 5.00 equiv) and HATU (61.20 mg, 0.161 mmol, 1.50 equiv). The mixture was stirred at room temperature for 1 hours. The crude product (55 mg) was purified by Prep-HPLC (conditions: SunFire C18 OBD Prep Column, 100 Å, 5 μm, 19 mm×250 mm; Mobile Phase A: Water (0.1% FA), Mobile Phase B:ACN; Flow rate:25 mL/minute; Gradient:9 B to 28 B in 13 minutes; 254 nm; R_{T}: 14.08 minutes) to afford N-[[2,6-dimethoxy-4-(2-methyl-1-oxo-2,7-naphthyridin-4-yl)phenyl]methyl]-5-[9-[2-(2,6-dioxopiperidin-3-yl)-1,3-dioxoisoindol-5-yl]-1-oxa-4,9-diazaspiro[5.5]undecan-4-yl]-N-methylpentanamide formic acid (8.2 mg, 8.68%) as a yellow solid. ¹H NMR (400 MHz, Methanol-d4) δ 9.50 (d, J=3.4 Hz, 1H), 8.65 (dd, J=12.4, 5.8 Hz, 1H), 8.39 (brs, 0.6H, FA), 7.74 (d, J=4.5 Hz, 1H), 7.66-7.57 (m, 2H), 7.28 (dd, J=12.7, 2.3 Hz, 1H), 7.22-7.14 (m, 1H), 6.80 (d, J=20.6 Hz, 2H), 5.04 (dt, J=12.8, 5.8 Hz, 1H), 4.75 (d, J=16.1 Hz, 2H), 3.90 (d, J=16.5 Hz, 6H), 3.87-3.82 (m, 2H), 3.74-3.63 (m, 5H), 3.32-3.26 (m, 2H), 2.92-2.82 (m, 2H), 2.78 (d, J=6.8 Hz, 4H), 2.73-2.53 (m, 7H), 2.47 (t, J=6.7 Hz, 1H), 2.17-2.01 (m, 3H), 1.82-1.62 (m, 6H). LCMS (ESI) m/z: [M+H]⁺=834.40.

### Example 79—Preparation of 2-([[2,6-dimethoxy-4-(2-methyl-1-oxo-2,7-naphthyridin-4-yl)phenyl]methyl](methyl)amino)-N-(4-[9-[2-(2,6-dioxopiperidin-3-yl)-1,3-dioxoisoindol-5-yl]-1-oxa-4,9-diaza spiro[5.5]undecan-4-yl]butyl)acetamide formic acid (compound D73 formic acid)

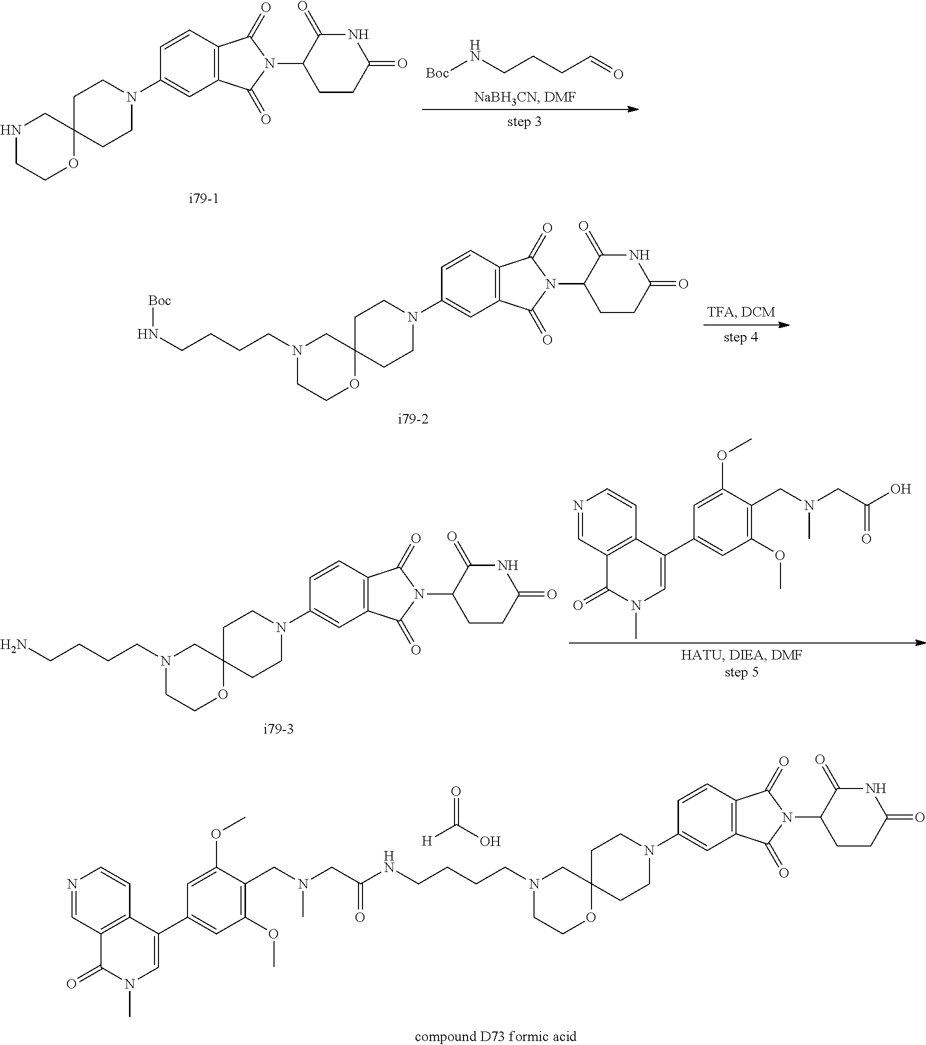

### Step 1: Preparation of tert-butyl N-(4-[9-[2-(2,6-dioxopiperidin-3-yl)-1,3-dioxoisoindol-5-yl]-1-oxa-4,9-diazaspiro[5.5]undecan-4-yl]butyl)carbamate (i79-2)

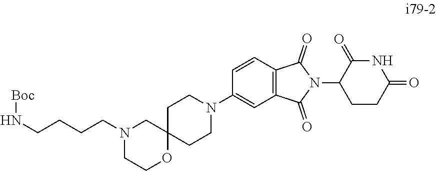 [SMILES: CC(C)(C)OC(=O)NCCCCN1CCOC2(CCN(c3ccc4c(c3)C(=O)N(C3CCC(=O)NC3=O)C4=O)CC2)C1]

To a stirred solution of 2-(2,6-dioxopiperidin-3-yl)-5-[1-oxa-4,9-diazaspiro[5.5]undecan-9-yl]isoindole-1,3-dione (200.00 mg, 0.485 mmol, 1.00 equiv) and tert-butyl N-(4-oxobutyl)carbamate (907.94 mg, 4.849 mmol, 10.00 equiv) in DMF (1.50 mL) was added NaBH₃CN (60.95 mg, 0.970 mmol, 2.00 equiv). The mixture was stirred at room temperature for 5 hours. The resulting mixture was concentrated under vacuum. The residue was purified by Prep-TLC (Petroleum ether/EtOAc 1:3) to afford tert-butyl N-(4-[9-[2-(2,6-dioxopiperidin-3-yl)-1,3-dioxoisoindol-5-yl]-1-oxa-4,9-diazaspiro[5.5]undecan-4-yl]butyl)carbamate(200 mg, crude) as a yellow solid. LCMS (ESI) m/z: [M+H]+=584.

### Step 2: Preparation of 5-[4-(4-aminobutyl)-1-oxa-4,9-diazaspiro[5.5]undecan-9-yl]-2-(2,6-dioxopiperidin-3-yl)isoindole-1,3-dione (i79-3)

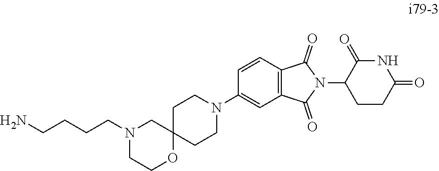 [SMILES: NCCCCN1CCOC2(CCN(c3ccc4c(c3)C(=O)N(C3CCC(=O)NC3=O)C4=O)CC2)C1]

To a stirred solution of tert-butyl N-(4-[9-[2-(2,6-dioxopiperidin-3-yl)-1,3-dioxoisoindol-5-yl]-1-oxa-4,9-diazaspiro[5.5]undecan-4-yl]butyl)carbamate (200.00 mg, 0.343 mmol, 1.00 equiv) in DCM (3.00 mL) was added TFA (1.00 mL). The mixture was stirred at room temperature for 2 hours. The residue was purified by Prep-TLC (CH₂Cl₂/MeOH 10:1) to afford 5-[4-(4-aminobutyl)-1-oxa-4,9-diazaspiro[5.5]undecan-9-yl]-2-(2,6-dioxopiperidin-3-yl)isoindole-1,3-dione (60 mg, 36.21%) as a yellow solid. LCMS (ESI) m/z: [M+H]+=484.

### Step 3: Preparation of 2-([[2,6-dimethoxy-4-(2-methyl-1-oxo-2,7-naphthyridin-4-yl)phenyl]methyl](methyl)amino)-N-(4-[9-[2-(2,6-dioxopiperidin-3-yl)-1,3-dioxoisoindol-5-yl]-1-oxa-4,9-diazaspiro[5.5]undecan-4-yl]butyl)acetamide formic acid (compound D73 formic acid)

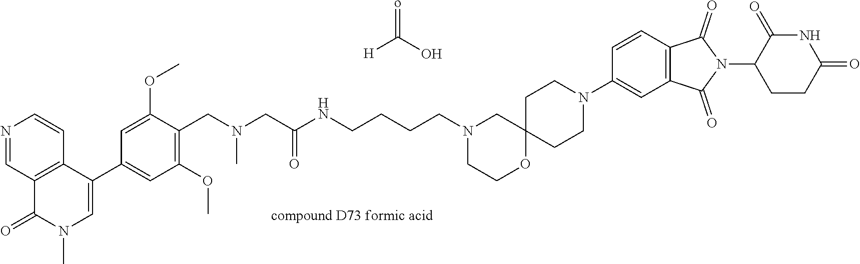

To a stirred solution of 5-[4-(4-aminobutyl)-1-oxa-4,9-diazaspiro[5.5]undecan-9-yl]-2-(2,6-dioxopiperidin-3-yl)isoindole-1,3-dione (60.00 mg, 0.124 mmol, 1.00 equiv) and ([[2,6-dimethoxy-4-(2-methyl-1-oxo-2,7-naphthyridin-4-yl)phenyl]methyl](methyl)amino)acetic acid (49.31 mg, 0.124 mmol, 1.00 equiv) in DMF (1.00 mg) was added DIEA (80.18 mg, 0.620 mmol, 5.00 equiv) and HATU (70.77 mg, 0.186 mmol, 1.50 equiv). The mixture was stirred at room temperature for 1 hour. The crude product (60 mg) was purified by Prep-HPLC (conditions: SunFire C18 OBD Prep Column, 100 Å, 5 μm, 19 mm×250 mm; Mobile Phase A: Water (0.1% FA), Mobile Phase B:ACN; Flow rate:25 mL/minute; Gradient:8 B to 17 B in 12 minutes; 254 nm; R_{T}: 11.87 minutes) to afford 2-([[2,6-dimethoxy-4-(2-methyl-1-oxo-2,7-naphthyridin-4-yl)phenyl]methyl](methyl)amino)-N-(4-[9-[2-(2,6-dioxopiperidin-3-yl)-1,3-dioxoisoindol-5-yl]-1-oxa-4,9-diazaspiro[5.5]undecan-4-yl]butyl)acetamide formic acid (12.6 mg, 10.72%) as a yellow solid. ¹H NMR (400 MHz, Methanol-d4) δ 9.51 (s, 1H), 8.68 (d, J=5.8 Hz, 1H), 8.53 (brs, 0.9H, FA), 7.74 (s, 1H), 7.62 (dd, J=7.3, 6.3 Hz, 2H), 7.27 (d, J=2.3 Hz, 1H), 7.16 (dd, J=8.6, 2.4 Hz, 1H), 6.82 (s, 2H), 5.05 (dd, J=12.7, 5.5 Hz, 1H), 4.15 (s, 2H), 3.94 (s, 6H), 3.75 (t, J=4.8 Hz, 2H), 3.69 (s, 3H), 3.64 (d, J=13.0 Hz, 2H), 3.54 (s, 2H), 3.31-3.25 (m, 4H), 2.94-2.81 (m, 1H), 2.80-2.68 (m, 2H), 2.63 (s, 3H), 2.46 (s, 2H), 2.37 (t, J=6.6 Hz, 2H), 2.32 (s, 2H), 2.17-2.00 (m, 3H), 1.68-1.51 (m, 6H). LCMS (ESI) m/z: [M+H]⁺=863.50.

### Example 80—Preparation of 5-[(1-[2-[2-([[2,6-dimethoxy-4-(2-methyl-1-oxo-2,7-naphthyridin-4-yl)phenyl]methyl]amino)ethoxy]acetyl]azetidin-3-yl)methoxy]-2-(2,6-dioxopiperidin-3-yl)isoindole-1,3-dione formic acid (compound D74 formic acid)

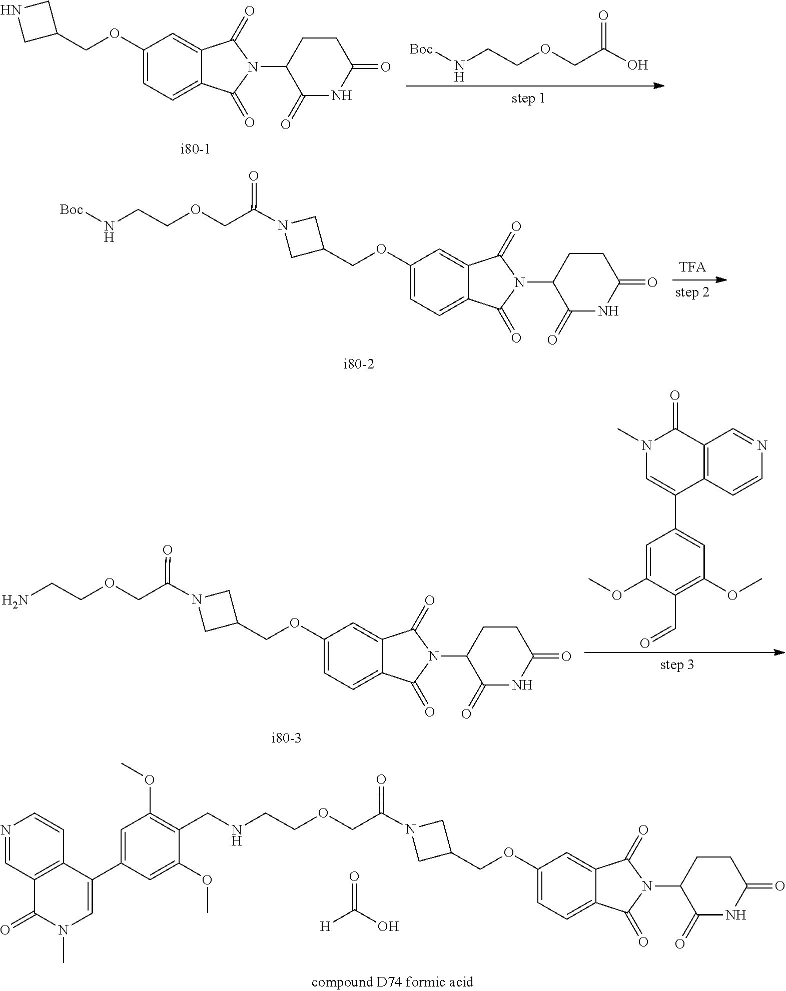

### Step 1: Preparation of tert-butyl N-(2-[2-[3-([[2-(2,6-dioxopiperidin-3-yl)-1,3-dioxoisoindol-5-yl]oxy]methyl) azetidin-1-yl]-2-oxoethoxy]ethyl)carbamate (i80-2)

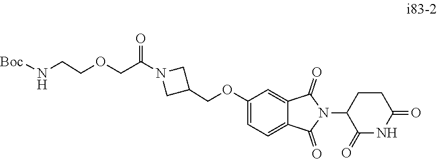 [SMILES: CC(C)(C)OC(=O)NCCOCC(=O)N1CC(COc2ccc3c(c2)C(=O)N(C2CCC(=O)NC2=O)C3=O)C1]

To a solution of [2-[(tert-butoxycarbonyl)amino]ethoxy]acetic acid (30.65 mg, 0.140 mmol, 1.20 equiv) and HATU (88.60 mg, 0.233 mmol, 2.00 equiv) in DMF (1.00 mL) was added 5-(azetidin-3-ylmethoxy)-2-(2,6-dioxopiperidin-3-yl)isoindole-1,3-dione (40.00 mg, 0.117 mmol, 1.00 equiv) and DIEA (45.17 mg, 0.350 mmol, 3.00 equiv), and the resulting solution was stirred at 25° C. for 2 hours. The resulting mixture was concentrated. The residue was applied onto a silica gel column with CH₂Cl₂/MeOH (20:1). This resulted in (50 mg, 78.81%) of tert-butyl N-(2-[2-[3-([[2-(2,6-dioxopiperidin-3-yl)-1,3-dioxoisoindol-5-yl]oxy]methyl)azetidin-1-yl]-2-oxoethoxy]ethyl)carbamate as a yellow solid. LCMS (ESI) m/z: [M+H]+=545.30.

### Step 2: Preparation of 5-([1-[2-(2-aminoethoxy)acetyl]azetidin-3-yl]methoxy)-2-(2,6-dioxopiperidin-3-yl)isoindole-1,3-dione (i80-3)

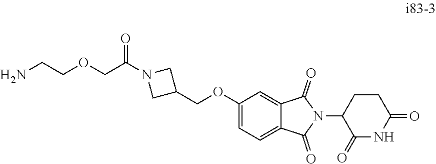 [SMILES: NCCOCC(=O)N1CC(COc2ccc3c(c2)C(=O)N(C2CCC(=O)NC2=O)C3=O)C1]

To a solution of tert-butyl N-(2-[2-[3-([[2-(2,6-dioxopiperidin-3-yl)-1,3-dioxoisoindol-5-yl]oxy]methyl) azetidin-1-yl]-2-oxoethoxy]ethyl)carbamate (50.00 mg, 0.092 mmol, 1.00 equiv) in TFA (2.00 mL) and DCM (2.00 mL), and the resulting solution was stirred at 25° C. for 2 hours. The resulting mixture was concentrated and used directly without further purification. This resulted in (60 mg, crude) of 5-([1-[2-(2-aminoethoxy)acetyl]azetidin-3-yl]methoxy)-2-(2,6-dioxopiperidin-3-yl)isoindole-1,3-dione as a yellow solid. LCMS (ESI) m/z: [M+H]+=445.50.

### Step 3: Preparation of 5-[(1-[2-[2-([[2,6-dimethoxy-4-(2-methyl-1-oxo-2,7-naphthyridin-4-yl)phenyl]methyl]amino)ethoxy]acetyl]azetidin-3-yl)methoxy]-2-(2,6-dioxopiperidin-3-yl)isoindole-1,3-dione formic acid (compound D74 formic acid)

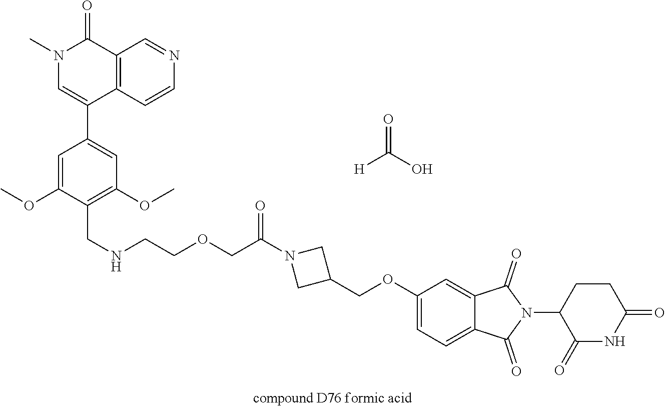 [SMILES: COc1cc(-c2cn(C)c(=O)c3cnccc23)cc(OC)c1CNCCOCC(=O)N1CC(COc2ccc3c(c2)C(=O)N(C2CCC(=O)NC2=O)C3=O)C1 ; O=CO]

To a solution of 5-([1-[2-(2-aminoethoxy)acetyl]azetidin-3-yl]methoxy)-2-(2,6-dioxopiperidin-3-yl)isoindole-1,3-dione (20 mg, 0.045 mmol, 1.00 equiv) and 2,6-dimethoxy-4-(2-methyl-1-oxo-2,7-naphthyridin-4-yl)benzaldehyde (17.51 mg, 0.054 mmol, 1.20 equiv) in DMF (2.00 mL) was added NaBH₃CN (5.66 mg, 0.090 mmol, 2.00 equiv). The resulting solution was stirred at 25° C. for 2 hours. The resulting mixture was concentrated. The crude product was purified by preparative HPLC Column: XSelect CSH Prep C18 OBD Column, 5 μm, 19*150 mm; Mobile Phase A: Water (0.1% FA), Mobile Phase B: ACN; Flow rate: 25 mL/minute; Gradient: 20% B to 55% B in 8 minutes; 254 nm; Rt: 7.12 minutes). This resulted in (10 mg, 27.82%) of 5-[(1-[2-[2-([[2,6-dimethoxy-4-(2-methyl-1-oxo-2,7-naphthyridin-4-yl)phenyl]methyl]amino)ethoxy]acetyl]azetidin-3-yl)methoxy]-2-(2,6-dioxopiperidin-3-yl)isoindole-1,3-dione as an off-white solid. ¹H NMR (400 MHz, Methanol-d4) δ 9.53 (s, 1H), 8.68 (d, J=5.8 Hz, 1H), 8.57 (brs, 3.2H, FA), 7.82 (d, J=8.3 Hz, 1H), 7.76 (s, 1H), 7.61 (d, J=5.7 Hz, 1H), 7.43 (d, J=2.3 Hz, 1H), 7.35 (dd, J=8.1, 2.3 Hz, 1H), 6.84 (s, 2H), 5.12 (dd, J=12.6, 5.4 Hz, 1H), 4.40 (t, J=8.8 Hz, 1H), 4.35 (d, J=3.8 Hz, 4H), 4.27-4.13 (m, 4H), 4.02-3.93 (m, 7H), 3.83 (t, J=4.9 Hz, 2H), 3.71 (s, 3H), 3.27-3.21 (m, 3H), 2.94-2.83 (m, 1H), 2.82-2.67 (m, 2H), 2.20-2.10 (m, 1H). LCMS (ESI) m/z: [M+H]+=753.40.

### Example 81—Preparation of Compounds 075-0177

In analogy to the procedures described in the examples above, compounds D75-D177 were prepared using the appropriate starting materials.

| Compound No. | Analytical Data |
|---|---|
| D75 | LCMS: (ESI) m/z: [M + H]⁺ = 835.70 |
| D76 | LCMS: (ESI) m/z: [M + H]⁺ = 788.20 |
| D77 | LCMS: (ESI) m/z: [M + H]⁺ = 774.10 |
| D78 | LCMS: 789.2; ¹H NMR (400 MHz, DMSO-d6) δ 11.11 (s, 1H), 9.45 (s, 1H), |
| | 8.73 (d, J = 5.7 Hz, 1H), 8.21 (s, 0.7H, FA), 8.05 (d, J = 7.5 Hz, 1H), 7.87 (s, |
| | 1H), 7.82 (d, J = 8.2 Hz, 1H), 7.59-7.54 (m, 1H), 7.31-7.24 (m, 2H), 6.73 |
| | (s, 2H), 5.12 (dd, J = 12.8, 5.4 Hz, 1H), 4.87 (t, J = 6.8 Hz, 1H), 4.14-3.99 |
| | (m, 1H), 3.81 (s, 6H), 3.66 (s, 2H), 3.61 (s, 3H), 3.44-3.35 (m, 3H), 2.98 |
| | (s, 2H), 2.92-2.83 (m, 1H), 2.73-2.55 (m, 4H), 2.44-2.32 (m, 1H), 2.25 |
| | (dd, J = 18.0, 6.8 Hz, 3H), 2.15-2.00 (m, 3H), 1.95 (td, J = 11.2, 8.4 Hz, 2H). |
| D79 | LCMS: (ESI) m/z: [M + H]⁺ = 789.20; ¹H NMR (400 MHz, DMSO-d6) δ 11.11 |
| | (s, 1H), 9.45 (s, 1H), 8.73 (d, 5.7 Hz, 1H), 8.21 (s, 0.7H, FA), 8.05 (d, J = |
| | 7.5 Hz, 1H), 7.87 (s, 1H), 7.82 (d, J = 8.2 Hz, 1H), 7.59-7.54 (m, 1H), |
| | 7.31-7.24 (m, 2H), 6.73 (s, 2H), 5.12 (dd, J = 12.8, 5.4 Hz, 1H), 4.87 (t, J = |
| | 6.8 Hz, 1H), 4.14-3.99 (m, 1H), 3.81 (s, 6H), 3.66 (s, 2H), 3.61 (s, 3H), |
| | 3.44-3.35 (m, 3H), 2.98 (s, 2H), 2.92-2.83 (m, 1H), 2.73-2.55 (m, 4H), |
| | 2.44-2.32 (m, 1H), 2.25 (dd, J = 18.0, 6.8 Hz, 3H), 2.15-2.00 (m, 3H), |
| | 1.95 (td, J = 11.2, 8.4 Hz, 2H). |
| D80 | LCMS: (ESI) m/z: [M + H]⁺ = 803.15; ¹H NMR (400 MHz, DMSO-d6) δ 11.12 |
| | (s, 1H), 9.45 (s, 1H), 8.73 (d, J = 5.6 Hz, 1H), 8.21 (s, 0.6H, FA), 7.97- |
| | 7.77 (m, 2H), 7.56 (d, J = 5.7 Hz, 1H), 7.37-7.20 (m, 2H), 6.74 (s, 2H), |
| | 5.12 (dd, J = 12.8, 5.4 Hz, 1H), 5.10-4.97 (m, 1H), 3.82 (d, J = 2.0 Hz, |
| | 6H), 3.70 (s, 2H), 3.61 (s, 3H), 3.36 (s, 5H), 3.09-2.95 (m, 2H), 2.88 (d, J = |
| | 13.9 Hz, 1H), 2.58 (d, J = 10.3 Hz, 8H), 2.16-1.99 (m, 1H), 1.87 (d, J = |
| | 9.8 Hz, 2H), 1.63 (s, 1H), 1.55 (s, 2H), 1.47 (s, 1H). |
| D81 | LCMS: (ESI) m/z: [M + H]⁺ = 715.20 |
| D82 | LCMS: (ESI) m/z: [M + H]⁺ = 821.25; ¹H NMR (300 MHz, Methanol-d4) δ 9.54 |
| | (d, J = 0.9 Hz, 1H), 8.69 (d, J = 5.8 Hz, 1H), 8.54 (s, 0.4H, FA), 7.86-7.75 |
| | (m, 2H), 7.63 (dd, J = 5.8, 0.9 Hz, 1H), 7.50 (dd, J = 7.8, 5.7 Hz, 2H), 6.87 |
| | (s, 2H), 5.14 (dd, J = 12.3, 5.4 Hz, 1H), 4.44-4.32 (m, 4H), 4.24 (p, J = 8.3 |
| | Hz, 1H), 3.97 (s, 6H), 3.93-3.83 (m, 4H), 3.72 (s, 3H), 3.22-3.02 (m, 2H), |
| | 2.99-2.65 (m, 4H), 2.46 (t, J = 5.9 Hz, 2H), 2.27 (s, 2H), 2.22-2.09 (m, |
| | 2H), 1.91 (s, 2H), 1.76 (s, 4H). |
| D83 | LCMS: (ESI) m/z: [M + H]⁺ = 781.55; ¹H NMR (300 MHz, Methanol-d4) δ 9.53 |
| | (s, 1H), 8.69 (d, J = 6.0 Hz, 1H), 7.85 (s, 1H), 7.82-7.66 (m, 2H), 7.50- |
| | 7.36 (m, 2H), 6.85 (d, J = 1.2 Hz, 2H), 5.12 (dd, J = 12.5, 5.4 Hz, 1H), 4.50 |
| | (s, 1H), 4.42 (s, 1H), 4.35-4.11 (m, 4H), 4.02 (dd, J = 11.0, 6.7 Hz, 1H), |
| | 3.94 (d, J = 2.7 Hz, 6H), 3.91-3.76 (m, 5H), 3.71 (d, J = 1.5 Hz, 3H), 3.22 |
| | (t, J = 6.5 Hz, 2H), 3.08-2.59 (m, 4H), 2.46 (t, J = 5.8 Hz, 2H), 2.21-2.07 |
| | (m, 1H), 1.84 (p, J = 7.2, 6.7 Hz, 2H). |
| D84 | LCMS: (ESI) m/z: [M + H]⁺ = 793.55 |
| D85 | LCMS: (ESI) m/z: [M + H]⁺ = 807.25 |
| D86 | LCMS: (ESI) m/z: [M + H]⁺ = 779.20 |
| D87 | LCMS: (ESI) m/z: [M + H]⁺ = 793.45 |
| D88 | LCMS: (ESI) m/z: [M + H]⁺ = 807.90; ¹H NMR (400 MHz, Methanol-d4) δ 9.55 |
| | (d, J = 0.8 Hz, 1H), 8.69 (d, J = 5.7 Hz, 1H), 8.56 (s, 0.5H, FA), 7.86-7.73 |
| | (m, 2H), 7.63 (d, J = 5.8 Hz, 1H), 7.48 (dd, J = 7.9, 6.2 Hz, 2H), 6.85 (s, |
| | 2H), 5.24-5.02 (m, 1H), 4.38 (t, J = 4.3 Hz, 2H), 4.33 (s, 2H), 3.95 (s, 6H), |
| | 3.93-3.81 (m, 4H), 3.72 (s, 4H), 3.71-3.40 (m, 4H), 3.25-3.01 (m, 3H), |
| | 2.98-2.82 (m, 2H), 2.82-2.61 (m, 3H), 2.21-2.07 (m, 1H), 1.91 (s, 2H), |
| | 1.63 (d, J = 17.7 Hz, 4H). |
| D89 | LCMS: (ESI) m/z: [M + H]⁺ = 821.30 |
| D90 | LCMS: (ESI) m/z: [M + H]⁺ = 793.45 |
| D91 | LCMS: (ESI) m/z: [M + H]⁺ = 807.50 |
| D92 | LCMS: (ESI) m/z: [M + H]⁺ = 793.60; ¹H NMR (300 MHz, Methanol-d4) δ 9.53 |
| | (s, 1H), 8.68 (dd, J = 5.8, 2.4 Hz, 1H), 8.52 (s, 0.5H, FA), 7.90-7.73 (m, |
| | 2H), 7.62 (s, 1H), 7.47 (dd, J = 9.3, 3.5 Hz, 2H), 6.89-6.74 (m, 2H), 5.24- |
| | 5.04 (m, 1H), 4.31 (d, J = 33.1 Hz, 5H), 3.90 (dd, J = 6.6, 4.5 Hz, 12H), 3.78- |
| | 3.58 (m, 7H), 3.00-2.48 (m, 6H), 2.26-1.78 (m, 3H). |
| D93 | LCMS: (ESI) m/z: [M + H]⁺ = 793.50 |
| D94 | LCMS: (ESI) m/z: [M + H]⁺ = 865.55 |
| D95 | LCMS: (ESI) m/z: [M + H]⁺ = 793.65 |
| D96 | LCMS: (ESI) m/z: [M + H]⁺ = 835.45 |
| D97 | LCMS: (ESI) m/z: [M + H]⁺ = 865.50; ¹H NMR (300 MHz, Methanol-d4) δ 9.54 |
| | (d, J = 0.8 Hz, 1H), 8.69 (d, J = 5.8 Hz, 1H), 8.55 (s, 0.6H, FA), 7.90-7.71 |
| | (m, 2H), 7.64 (d, J = 5.8 Hz, 1H), 7.48 (dd, J = 7.9, 3.5 Hz, 2H), 6.87 (s, |
| | 2H), 5.12 (dd, J = 12.3, 5.4 Hz, 1H), 4.50-4.23 (m, 4H), 3.97 (s, 6H), 3.95- |
| | 3.79 (m, 5H), 3.72 (s, 5H), 3.66 (dd, J = 5.8, 1.9 Hz, 1H), 3.59-3.32 (m, |
| | 3H), 3.30-2.98 (m, 2H), 2.98-2.59 (m, 6H), 2.25-1.70 (m, 7H), 1.49 (s, 2H). |
| D98 | LCMS: (ESI) m/z: [M + H]⁺ = 779.40; ¹H NMR (400 MHz, Methanol-d4) δ 9.51 |
| | (d, J = 1.5 Hz, 1H), 8.67 (d, J = 5.7 Hz, 1H), 7.69 (d, J = 1.8 Hz, 1H), 7.63- |
| | 7.49 (m, 2H), 7.27 (dd, J = 5.8, 2.3 Hz, 1H), 7.16 (ddd, J = 11.0, 8.4, 2.3 Hz, |
| | 1H), 6.65 (d, J = 2.1 Hz, 2H), 5.04 (td, J = 12.4, 5.5 Hz, 1H), 4.80 (d, J = |
| | 10.2 Hz, 1H), 4.33 (d, J = 10.5 Hz, 2H), 4.25 (d, J = 16.3 Hz, 2H), 4.03 (d, J = |
| | 11.4 Hz, 1H), 3.95-3.74 (m, 12H), 3.74 (d, J = 1.3 Hz, 4H), 3.39-3.30 |
| | (m, 1H), 2.80 (dt, J = 13.9, 4.7 Hz, 1H), 2.76-2.54 (m, 3H), 2.46-2.22 (m, |
| | 3H), 2.03 (td, J = 7.3, 6.8, 3.3 Hz, 1H). |
| D99 | LCMS: (ESI) m/z: [M + H]⁺ = 793.45 |
| D100 | LCMS: (ESI) m/z: [M + H]⁺ = 793.35 |
| D101 | LCMS: (ESI) m/z: [M + H]⁺ = 793.45; ¹H NMR (300 MHz, Methanol-d4) δ 9.53 |
| | (d, J = 0.8 Hz, 1H), 8.69 (d, J = 5.8 Hz, 1H), 8.56 (s, 0.7H, FA), 7.86-7.73 |
| | (m, 2H), 7.64-7.56 (m, 1H), 7.43 (d, J = 2.3 Hz, 1H), 7.34 (dd, J = 8.3, 2.3 |
| | Hz, 1H), 6.84 (s, 2H), 5.12 (dd, J = 12.4, 5.4 Hz, 1H), 4.36-4.20 (m, 4H), |
| | 4.20-4.05 (m, 3H), 3.96 (d, J = 8.5 Hz, 8H), 3.90-3.75 (m, 4H), 3.71 (s, |
| | 3H), 2.97-2.55 (m, 5H), 2.43 (t, J = 5.9 Hz, 2H), 2.25-2.08 (m, 3H). |
| D102 | LCMS: (ESI) m/z: [M + H]⁺ = 761.2 |
| D103 | LCMS: (ESI) m/z: [M + H]⁺ = 747.3 |
| D104 | LCMS: (ESI) m/z: [M + H]⁺ = 747.3 |
| D105 | LCMS: (ESI) m/z: [M + H]⁺ = 719.3 |
| D106 | LCMS: (ESI) m/z: [M + H]⁺ = 733.4 |
| D107 | LCMS: (ESI) m/z: [M + H]⁺ = 733.3 |
| D108 | LCMS: (ESI) m/z: [M + H]⁺ = 807.45 |
| D109 | LCMS: (ESI) m/z: [M + H]⁺ = 865.35 |
| D110 | LCMS: (ESI) m/z: [M + H]⁺ = 835.75 |
| D111 | LCMS: (ESI) m/z: [M + H]⁺ = 793.50 |
| D112 | LCMS: (ESI) m/z: [M + H]⁺ = 793.50 |
| D113 | LCMS: (ESI) m/z: [M + H]⁺ = 779.35 |
| D114 | LCMS: (ESI) m/z: [M + H]⁺ = 851.25 |
| D115 | LCMS: (ESI) m/z: [M + H]⁺ = 793.45 |
| D116 | LCMS: (ESI) m/z: [M + H]⁺ = 821.30 |
| D117 | LCMS: (ESI) m/z: [M + H]⁺ = 781.60; ¹H NMR (300 MHz, Methanol-d4) δ 9.51 |
| | (s, 1H), 8.68 (d, J = 5.8 Hz, 1H), 8.56 (s, 0.7H, FA), 7.76 (d, J = 8.6 Hz, 2H), |
| | 7.60 (d, J = 5.8 Hz, 1H), 7.39 (d, J = 2.2 Hz, 1H), 7.30 (dd, J = 8.3, 2.3 Hz, |
| | 1H), 6.84 (s, 2H), 5.10 (dd, J = 12.5, 5.4 Hz, 1H), 4.37 (s, 2H), 4.33-4.24 |
| | (m, 2H), 4.22-4.08 (m, 2H), 3.95 (s, 6H), 3.85 (dq, J = 7.2, 5.7 Hz, 6H), |
| | 3.70 (s, 3H), 3.20 (t, J = 6.5 Hz, 2H), 3.02-2.62 (m, 4H), 2.47 (t, J = 5.8 |
| | Hz, 2H), 2.23-2.05 (m, 1H), 1.84 (q, J = 6.9 Hz, 2H). |
| D118 | LCMS: (ESI) m/z: [M + H]⁺ = 807.60; ¹H NMR (300 MHz, Methanol-d4) δ 9.53 |
| | (d, J = 0.8 Hz, 1H), 8.69 (d, J = 5.8 Hz, 1H), 8.55 (s, 0.7H, FA), 7.89-7.75 |
| | (m, 2H), 7.61 (dd, J = 5.8, 0.8 Hz, 1H), 7.44 (d, J = 2.2 Hz, 1H), 7.37-7.30 |
| | (m, 1H), 6.85 (s, 2H), 5.10 (dd, J = 12.4, 5.4 Hz, 1H), 4.41 (s, 2H), 4.35- |
| | 4.25 (m, 2H), 3.95 (s, 6H), 3.91-3.77 (m, 8H), 3.72 (s, 3H), 3.54 (q, J = 5.6 |
| | Hz, 4H), 2.96-2.63 (m, 5H), 2.12 (dtd, J = 12.8, 4.8, 2.1 Hz, 1H), 1.83 (dt, |
| | J = 16.1, 5.8 Hz, 4H). |
| D119 | LCMS: (ESI) m/z: [M + H]⁺ = 807.45 |
| D120 | LCMS: (ESI) m/z: [M + H]⁺ = 821.45 |
| D121 | LCMS: (ESI) m/z: [M + H]⁺ = 807.40; ¹H NMR (400 MHz, Methanol-d4) δ 9.53 |
| | (d, J = 1.0 Hz, 1H), 8.69 (d, J = 5.7 Hz, 1H), 8.54 (s, 0.5H, FA), 7.88-7.73 |
| | (m, 2H), 7.66-7.59 (m, 1H), 7.41 (dd, J = 4.4, 2.3 Hz, 1H), 7.32 (ddd, J = |
| | 8.1, 6.0, 2.1 Hz, 1H), 6.84 (d, J = 7.6 Hz, 2H), 5.10 (dd, J = 6.9, 5.4 Hz, 1H), |
| | 4.38 (s, 1H), 4.30 (d, J = 4.9 Hz, 3H), 3.95 (d, J = 8.8 Hz, 6H), 3.87 (t, J = |
| | 4.6 Hz, 4H), 3.71 (d, J = 1.2 Hz, 3H), 3.71-3.56 (m, 2H), 3.55-3.37 (m, |
| | 3H), 3.33-3.26 (m, 3H), 2.97-2.52 (m, 5H), 2.21-1.92 (m, 5H). |
| D122 | LCMS: (ESI) m/z: [M + H]⁺ = 793.35; ¹H NMR (400 MHz, Methanol-d4) δ 9.53 |
| | (d, J = 2.5 Hz, 1H), 8.68 (dd, J = 5.7, 1.6 Hz, 1H), 8.54 (s, 0.6H, FA), 7.85- |
| | 7.70 (m, 2H), 7.60 (dd, J = 6.0, 3.1 Hz, 1H), 7.42 (dd, J = 3.5, 2.2 Hz, 1H), |
| | 7.37-7.29 (m, 1H), 6.84 (d, J = 9.2 Hz, 2H), 5.11 (dd, J = 12.5, 5.4 Hz, |
| | 1H), 4.40 (s, 1H), 4.31 (dt, J = 6.1, 3.1 Hz, 3H), 4.08-4.00 (m, 2H), 3.99- |
| | 3.91 (m, 8H), 3.90-3.82 (m, 4H), 3.81 (s, 1H), 3.71 (d, J = 1.2 Hz, 3H), |
| | 3.68-3.58 (m, 2H), 3.47 (t, J = 7.1 Hz, 1H), 2.96-2.81 (m, 1H), 2.74 (dtt, |
| | J = 12.1, 6.1, 3.4 Hz, 2H), 2.62 (dt, J = 11.5, 5.9 Hz, 2H), 2.25 (t, J = 7.0 Hz, |
| | 1H), 2.22-2.05 (m, 2H). |
| D123 | LCMS: (ESI) m/z: [M + H]⁺ = 807.30 |
| D124 | LCMS: (ESI) m/z: [M + H]⁺ = 865.90 |
| D125 | LCMS: (ESI) m/z: [M + H]⁺ = 793.20 |
| D126 | LCMS: (ESI) m/z: [M + H]⁺ = 793.20 |
| D127 | LCMS: (ESI) m/z: [M + H]⁺ = 793.55 |
| D128 | LCMS: (ESI) m/z: [M + H]⁺ = 779.40 |
| D129 | LCMS: (ESI) m/z: [M + H]⁺ = 835.70 |
| D130 | LCMS: (ESI) m/z: [M + H]⁺ = 851.40 |
| D131 | LCMS: (ESI) m/z: [M + H]⁺ = 865.35 |
| D132 | LCMS: (ESI) m/z: [M + H]⁺ = 775.3 |
| D133 | LCMS: (ESI) m/z: [M + H]⁺ = 777.5 |
| D134 | LCMS: (ESI) m/z: [M + H]⁺ = 761.4 |
| D135 | LCMS: (ESI) m/z: [M + H]⁺ = 763.4 |
| D136 | LCMS: (ESI) m/z: [M + H]⁺ = 775.2 |
| D137 | LCMS: (ESI) m/z: [M + H]⁺ = 789.3 |
| D138 | LCMS: (ESI) m/z: [M + H]⁺ = 803.5 |
| D139 | LCMS: (ESI) m/z: [M + H]⁺ = 805.4 |
| DUO | LCMS: (ESI) m/z: [M + H]⁺ = 775.2 |
| D141 | LCMS: (ESI) m/z: [M + H]⁺ = 789.3 |
| D142 | LCMS: (ESI) m/z: [M + H]⁺ = 803.5 |
| D143 | LCMS: (ESI) m/z: [M + H]⁺ = 817.5 |
| D144 | LCMS: (ESI) m/z: [M + H]⁺ = 819.3 |
| D145 | LCMS: (ESI) m/z: [M + H]⁺ = 689.3 |
| D146 | LCMS: (ESI) m/z: [M + H]⁺ = 717.3 |
| D147 | LCMS: (ESI) m/z: [M + H]⁺ = 731.4 |
| D148 | LCMS: (ESI) m/z: [M + H]⁺ = 745.2 |
| D149 | LCMS: (ESI) m/z: [M + H]⁺ = 745.3 |
| D150 | LCMS: (ESI) m/z: [M + H]⁺ = 789.5 |
| D151 | LCMS: (ESI) m/z: [M + H]⁺ = 805.9 |
| D152 | LCMS: (ESI) m/z: [M + H]⁺ = 831.4 |
| D153 | LCMS: (ESI) m/z: [M + H]⁺ = 833.3 |
| D154 | LCMS: (ESI) m/z: [M + H]⁺ = 789.3 |
| D155 | LCMS: (ESI) m/z: [M + H]⁺ = 803.2 |
| D156 | LCMS: (ESI) m/z: [M + H]⁺ = 817.6 |
| D157 | LCMS: (ESI) m/z: [M + H]⁺ = 831.6 |
| D158 | LCMS: (ESI) m/z: [M + H]⁺ = 833.5 |
| D159 | LCMS: (ESI) m/z: [M + H]⁺ = 851.25 |
| D160 | LCMS: (ESI) m/z: [M + H]⁺ = 821.45 |
| D161 | LCMS: (ESI) m/z: [M + H]⁺ = 821.35 |
| D162 | LCMS: (ESI) m/z: [M + H]⁺ = 807.35 |
| D163 | LCMS: (ESI) m/z: [M + H]⁺ = 835.50 |
| D164 | LCMS: (ESI) m/z: [M + H]⁺ = 821.60 |
| D165 | LCMS: (ESI) m/z: [M + H]⁺ = 849.60; ¹H NMR (300 MHz, Methanol-d4) δ 9.60- |
| | 9.41 (m, 1H), 8.69 (dd, J = 5.6, 3.0 Hz, 1H), 8.53 (s, 0.6H, FA), 7.79- |
| | 7.50 (m, 3H), 7.44-7.15 (m, 2H), 6.81-6.47 (m, 2H), 5.11 (dt, J = 11.6, |
| | 4.5 Hz, 1H), 4.57-4.07 (m, 5H), 4.05-3.76 (m, 13H), 3.74-3.66 (m, 3H), |
| | 3.64-3.44 (m, 1H), 3.05-2.65 (m, 5H), 2.64-2.02 (m, 6H). |
| D166 | LCMS: (ESI) m/z: [M + H]⁺ = 835.65 |
| D167 | LCMS: (ESI) m/z: [M + H]⁺ = 851.25 |
| D168 | LCMS: (ESI) m/z: [M + H]⁺ = 851.25 |
| D169 | LCMS: (ESI) m/z: [M + H]⁺ = 821.35 |
| D170 | LCMS: (ESI) m/z: [M + H]⁺ = 821.35 |
| D171 | LCMS: (ESI) m/z: [M + H]⁺ = 807.35 |
| D172 | LCMS: (ESI) m/z: [M + H]⁺ = 835.35 |
| D173 | LCMS: (ESI) m/z: [M + H]⁺ = 835.60 |
| D174 | LCMS: (ESI) m/z: [M + H]⁺ = 821.65 |
| D175 | LCMS: (ESI) m/z: [M + H]⁺ = 849.80 |
| D176 | LCMS: (ESI) m/z: [M + H]⁺ = 835.70 |
| D177 | LCMS: (ESI) m/z: [M + H]⁺ = 835.65 |

### Example 82—Preparation of Compounds 0178-037

In analogy to the procedures described in the examples above, compounds D178-D371 were prepared using the appropriate starting materials.

| Compound | | |
|---|---|---|
| No. | LCMS | ¹H NMR |
| D178 | 723.4 | ¹H NMR (300 MHz, DMSO-d6) δ 1.55 (2H, d), 1.77 (2H, d), 2.03 |
| | | (3H, d), 2.16 (3H, s), 2.44 (3H, d), 2.73 (2H, s), 2.88-3.08 (3H, |
| | | m), 3.61 (5H, s), 3.80 (6H, s), 4.30 (2H, s), 5.12 (1H, m), 6.72 (2H, |
| | | s), 7.38 (1H, m), 7.48 (1H, d), 7.57 (1H, d), 7.80-7.90 (2H, m), |
| | | 8.23 (1H, s), 8.72 (1H, d), 9.45 (1H, s), 11.12 (1H, s). |
| D179 | 813.3 | ¹H NMR(400 MHz, D .79 (brs, 0.8H, FA(COOH), 11.08 (s, 1H), |
| | | 9.44 (s, 1H), 8.71 (d, J = 5.7 Hz, 1H), 8.14 (s, 0.8H, FA), 7.86 (s, |
| | | 1H), 7.66 (d, J = 8.5 Hz, 1H), 7.56 (d, J = 5.8 Hz, 1H), 7.33 (d, J = |
| | | 2.3 Hz, 1H), 7.24 (dd, J = 8.8, 2.3 Hz, 1H), 7.11 (s, 1H), 6.73 (s, |
| | | 2H), 5.07 (dd, J = 13.0, 5.4 Hz, 1H), 4.08-4.02 (m, 1H), 3.82 (s, |
| | | 7H), 3.69-3.62 (m, 2H), 3.60 (s, 3H), 3.50-3.39 (m, 8H), 3.12- |
| | | 3.05 (m, 2H), 2.95-2.83 (m, 1H), 2.63-2.55 (m, 3H), 2.55 (s, |
| | | 2H), 2.47-2.39 (m, 3H), 2.07-1.98 (m, 1H). |
| D180 | 788.2 | |
| D181 | 774.7 | |
| D182 | 789.2 | ¹H NMR (400 MHz, DMSO-d6) δ 11.11 (s, 1H), 9.45 (s, 1H), 8.73 |
| | | (d, J = 5.7 Hz, 1H), 8.21 (s, 0.7H, FA), 8.05 (d, J = 7.5 Hz, 1H), |
| | | 7.87 (s, 1H), 7.82 (d, J = 8.2 Hz, 1H), 7.59-7.54 (m, 1H), 7.31- |
| | | 7.24 (m, 2H), 6.73 (s, 2H), 5.12 (dd, J = 12.8, 5.4 Hz, 1H), 4.87 (t, J = |
| | | 6.8 Hz, 1H), 4.14-3.99 (m, 1H), 3.81 (s, 6H), 3.66 (s, 2H), 3.61 |
| | | (s, 3H), 3.44-3.35 (m, 3H), 2.98 (s, 2H), 2.92-2.83 (m, 1H), 2.73- |
| | | 2.55 (m, 4H), 2.44-2.32 (m, 1H), 2.25 (dd, J = 18.0, 6.8 Hz, 3H), |
| | | 2.15-2.00 (m, 3H), 1.95 (td, J = 11.2, 8.4 Hz, 2H). |
| D183 | 789.5 | |
| D184 | 803.15 | |
| D185 | 715.2 | |
| D186 | 804.65 | ¹H NMR (400 MHz, DMSO-d6) δ 11.08 (s, 1H), 9.45 (d, J = 4.3 Hz, |
| | | 1H), 8.73 (d, J = 5.7 Hz, 1H), 8.16 (s, 0.6H, FA), 7.90 (d, J = 6.4 |
| | | Hz, 1H), 7.64 (dd, J = 8.3, 2.2 Hz, 1H), 7.58 (d, J = 5.7 Hz, 1H), |
| | | 6.90-6.72 (m, 3H), 6.65 (dd, J = 8.5, 2.3 Hz, 1H), 5.06 (dd, J = |
| | | 12.9, 5.4 Hz, 1H), 4.57 (d, J = 23.1 Hz, 2H), 3.83 (d, J = 18.2 Hz, |
| | | 6H), 3.74 (s, 4H), 3.60 (d, J = 3.3 Hz, 3H), 2.88 (ddd, J = 17.7, |
| | | 14.0, 5.4 Hz, 1H), 2.72 (s, 1H), 2.65 (s, 2H), 2.62-2.53 (m, 4H), |
| | | 2.44-2.26 (m, 6H), 2.08-1.94 (m, 1H), 1.77 (d, J = 6.5 Hz, 4H), |
| | | 1.53 (s, 4H). |
| D187 | 790.5 | |
| D188 | 804.6 | |
| D189 | 802.65 | |
| D190 | 788.6 | |
| D191 | 802.55 | |
| D192 | 788.8 | |
| D193 | 774.55 | |
| D194 | 774.75 | ¹H NMR (400 MHz, DMSO-d6) δ 11.08 (s, 1H), 9.45 (s, 1H), 8.73 |
| | | (d, J = 5.7 Hz, 1H), 8.22 (s, 1H, FA), 8.12 (d, J = 7.4 Hz, 1H), 7.88 |
| | | (s, 1H), 7.64 (d, J = 8.2 Hz, 1H), 7.57 (d, J = 5.7 Hz, 1H), 6.74 (d, J = |
| | | 6.3 Hz, 3H), 6.67-6.56 (m, 1H), 5.06 (dd, J = 12.8, 5.4 Hz, 1H), |
| | | 4.08 (d, J = 10.2 Hz, 3H), 3.97 (s, 2H), 3.82 (s, 6H), 3.67 (s, 2H), |
| | | 3.61 (s, 3H), 3.51 (s, 2H), 3.01 (d, J = 7.1 Hz, 2H), 2.95-2.80 (m, |
| | | 1H), 2.65-2.53 (m, 5H), 2.29 (d, J = 7.6 Hz, 2H), 2.12 (t, J = 10.3 |
| | | Hz, 2H), 2.07-1.93 (m, 1H). |
| D195 | 760.5 | ¹H NMR (400 MHz, DMSO-d6) δ 11.08 (s, 1H), 9.45 (s, 1H), 8.74 |
| | | (d, J = 5.6 Hz, 1H), 7.89 (s, 1H), 7.67 (d, J = 8.3 Hz, 1H), 7.56 (d, J = |
| | | 5.6 Hz, 1H), 6.82 (d, J = 2.4 Hz, 3H), 6.68 (dd, J = 8.4, 2.1 Hz, |
| | | 1H), 5.07 (dd, J = 12.9, 5.4 Hz, 1H), 4.32 (s, 2H), 4.20 (s, 6H), 4.06 |
| | | (s, 3H), 3.88 (s, 8H), 3.61 (s, 4H), 2.98-2.74 (m, 2H), 2.59 (d, J = |
| | | 16.5 Hz, 2H), 2.44 (d, J = 7.2 Hz, 2H), 2.11-1.95 (m, 1H). |
| D196 | 757.5 | |
| D197 | 743.35 | |
| D198 | 743.25 | |
| D199 | 731.35 | ¹H NMR (400 MHz, DMSO-d6) δ 11.15 (s, 1H), 9.52 (s, 1H), 8.77 |
| | | (d, J = 6.0 Hz, 1H), 8.06 (s, 1H), 7.91 (d, J = 8.1 Hz, 1H), 7.85 (dd, |
| | | J = 5.5, 1.6 Hz, 2H), 7.75 (d, J = 6.0 Hz, 1H), 6.87 (s, 2H), 5.16 (dd, |
| | | J = 12.8, 5.4 Hz, 1H), 4.43 (d, J = 13.9 Hz, 1H), 4.29 (s, 2H), 4.14- |
| | | 4.03 (m, 1H), 3.91 (s, 6H), 3.64 (s, 4H), 3.40 (t, J = 8.2 Hz, 2H), |
| | | 3.18 (m, 2H), 3.23-3.13 (m, 2H), 3.02-2.72 (m, 4H), 2.68-2.56 |
| | | (m, 2H), 2.12-1.99 (m, 1H). |
| D200 | 743.15 | |
| D201 | 804.7 | ¹H NMR (300 MHz, DMSO-d6) δ 11.09 (s, 1H), 9.45 (t, J = 1.4 Hz, |
| | | 1H), 8.72 (d, J = 5.7 Hz, 1H), 8.23 (s, 0.8H, FA), 7.90 (d, J = 6.0 |
| | | Hz, 1H), 7.76-7.56 (m, 2H), 7.40-7.16 (m, 2H), 6.77 (d, J = 10.7 |
| | | Hz, 2H), 5.17-4.99 (m, 1H), 4.56 (d, J = 19.2 Hz, 2H), 3.83 (d, J = |
| | | 13.5 Hz, 6H), 3.60 (d, J = 2.3 Hz, 3H), 3.43 (s, 6H), 3.01 (d, J = 5.0 |
| | | Hz, 4H), 2.98-2.78 (m, 1H), 2.72 (d, J = 5.9 Hz, 1H), 2.65 (s, 2H), |
| | | 2.63-2.55 (m, 1H), 2.47 (s, 2H), 2.27 (dd, J = 4.3, 2.4 Hz, 1H), |
| | | 2.10-1.91 (m, 1H), 1.73 (d, J = 6.4 Hz, 4H), 1.62-1.47 (m, 2H), |
| | | 1.44-1.26 (m, 2H). |
| D202 | 818.4 | |
| D203 | 790.6 | |
| D204 | 790.8 | |
| D205 | 776.35 | |
| D206 | 776.6 | |
| D207 | 805.65 | |
| D208 | 819.55 | ¹H NMR (300 MHz, DMSO-d6) δ 11.12 (s, 1H), 9.45 (s, 1H), 8.72 |
| | | (dd, J = 5.7, 2.2 Hz, 1H), 8.20 (s, 0.6H, FA), 7.90 (d, J = 3.6 Hz, |
| | | 1H), 7.83 (d, J = 8.2 Hz, 1H), 7.58 (dt, J = 5.7, 1.2 Hz, 1H), 7.37- |
| | | 7.22 (m, 2H), 6.77 (d, J = 9.7 Hz, 2H), 5.12 (dd, J = 12.9, 5.4 Hz, |
| | | 1H), 5.04-4.92 (m, 1H), 4.56 (d, J = 17.5 Hz, 2H), 3.82 (d, J = |
| | | 13.3 Hz, 6H), 3.60 (s, 3H), 2.90 (ddd, J = 17.3, 13.9, 5.4 Hz, 1H), |
| | | 2.71 (s, 1H), 2.61 (d, J = 20.0 Hz, 5H), 2.47-2.22 (m, 9H), 2.06 (d, |
| | | J = 5.9 Hz, 1H), 1.80 (dd, J = 12.2, 6.3 Hz, 2H), 1.70-1.43 (m, 8H). |
| D209 | 774.6 | |
| D210 | 760.7 | |
| D211 | 743.35 | ¹H NMR (400 MHz, DMSO-d6) δ 11.15 (s, 1H), 9.44 (s, 1H), 8.72 |
| | | (d, J = 5.7 Hz, 1H), 8.21 (s, 1H), 7.94-7.79 (m, 4H), 7.56 (d, J = |
| | | 5.7 Hz, 1H), 6.75 (s, 2H), 5.16 (dd, J = 12.8, 5.4 Hz, 1H), 3.82 (d, J = |
| | | 9.2 Hz, 8H), 3.60 (s, 4H), 3.46-3.40 (m, 6H), 2.90 (ddd, J = 16.9, |
| | | 13.8, 5.4 Hz, 1H), 2.70 (s, 2H), 2.66-2.53 (m, 5H), 2.07 (ddd, J = |
| | | 13.3, 5.6, 3.2 Hz, 1H), 1.94 (t, J = 7.0 Hz, 2H), 1.74 (p, J = 7.1 Hz, 2H). |
| D212 | 757.35 | |
| D213 | 771.2 | |
| D214 | 717.35 | ¹H NMR (400 MHz, DMSO-d6) δ 11.15 (s, 1H), 9.45 (s, 1H), 8.73 |
| | | (d, J = 5.7 Hz, 1H), 8.19 (s, 1H FA), 7.87 (d, J = 9.1 Hz, 4H), 7.58 |
| | | (d, J = 5.6 Hz, 1H), 6.74 (s, 2H), 5.16 (dd, J = 12.8, 5.4 Hz, 1H), |
| | | 3.81 (s, 6H), 3.60 (s, 6H), 3.47 (s, 5H), 2.94-2.85 (m, 1H), 2.68- |
| | | 2.58 (m, 2H), 2.44 (t, J = 7.2 Hz, 6H), 2.12-2.01 (m, 1H), 1.73 (p, |
| | | J = 7.1 Hz, 2H). |
| D215 | 729.35 | |
| D216 | 703.15 | |
| D217 | 771.15 | ¹H NMR (300 MHz, DMSO-d6) δ 11.15 (s, 1H), 9.47 (s, 1H), 8.75 |
| | | (d, J = 5.7 Hz, 1H), 7.97-7.79 (m, 4H), 7.58 (d, J = 5.6 Hz, 1H), |
| | | 6.87 (s, 2H), 5.16 (dd, J = 12.9, 5.3 Hz, 1H), 4.27 (d, J = 4.0 Hz, |
| | | 2H), 4.02 (s, 1H), 3.90 (s, 7H), 3.75 (s, 1H), 3.62 (s, 4H), 3.11 (s, |
| | | 2H), 3.08 (s, 2H), 2.96-2.84 (m, 1H), 2.69 (dd, J = 7.2, 3.6 Hz, |
| | | 2H), 2.66-2.54 (m, 2H), 2.47-2.39 (m, 2H), 2.13-2.00 (m, 3H), |
| | | 1.92 (t, J = 12.4 Hz, 2H). |
| D218 | 757.35 | |
| D219 | 771.35 | ¹H NMR (300 MHz, DMSO-d6) δ 11.15 (s, 1H), 9.45 (s, 1H), 8.73 |
| | | (d, J = 5.7 Hz, 1H), 8.20 (s, 1H FA), 7.97-7.77 (m, 4H), 7.57 (d, J = |
| | | 5.7 Hz, 1H), 6.74 (s, 2H), 5.24-5.08 (m, 1H), 3.82 (s, 6H), 3.71 |
| | | (s, 3H), 3.61 (s, 4H), 3.11 (s, 4H), 2.98-2.80 (m, 2H), 2.76-2.62 |
| | | (m, 6H), 2.15-2.01 (m, 1H), 1.61 (d, J = 27.8 Hz, 5H) |
| D220 | 785.15 | ¹H NMR (400 MHz, DMSO-d6) δ 11.15 (s, 1H), 9.45 (d, J = 2.0 Hz, |
| | | 1H), 8.73 (dd, J = 5.6, 2.2 Hz, 1H), 7.94-7.87 (m, 2H), 7.84 (q, J = |
| | | 2.9 Hz, 2H), 7.58 (dd, J = 5.7, 2.6 Hz, 1H), 6.74 (s, 2H), 5.16 (dd, J = |
| | | 12.8, 5.4 Hz, 1H), 3.82 (s, 6H), 3.60 (d, J = 1.4 Hz, 5H), 3.52 (t, J = |
| | | 7.0 Hz, 1H), 3.17 (s, 2H), 2.89 (ddd, J = 16.6, 13.6, 5.4 Hz, 1H), |
| | | 2.70 (t, J = 7.0 Hz, 2H), 2.66-2.56 (m, 7H), 2.41 (s, 2H), 2.12- |
| | | 2.00 (m, 1H), 1.76 (t, J = 7.1 Hz, 1H), 1.67 (t, J = 7.2 Hz, 1H), 1.49 |
| | | (s, 4H). |
| D221 | 817.35 | ¹H NMR (300 MHz, DMSO-d6) δ 11.13 (s, 1H), 9.45 (s, 1H), 8.73 |
| | | (d, J = 5.7 Hz, 1H), 8.20 (s, 1H, FA), 7.92-7.80 (m, 2H), 7.60 (d, J = |
| | | 5.7 Hz, 1H), 7.38-7.21 (m, 2H), 6.74 (s, 2H), 5.12 (dd, J = 12.9, |
| | | 5.4 Hz, 1H), 5.03 (t, J = 6.9 Hz, 1H), 3.81 (s, 6H), 3.61 (s, 3H), 3.58 |
| | | (s, 2H), 3.44 (s, 4H), 2.96-2.82 (m, 3H), 2.66-2.54 (m, 5H), 2.21- |
| | | 1.98 (m, 3H), 1.93-1.81 (m, 2H), 1.66-1.43 (m, 8H). |
| D222 | 776.4 | |
| D223 | 776.35 | |
| D224 | 790.4 | |
| D225 | 776.35 | |
| D226 | 762.8 | |
| D227 | 748.3 | ¹H NMR (300 MHz, DMSO-d6) δ 11.07 (s, 1H), 9.44 (s, 1H), 8.72 |
| | | (d, J = 5.7 Hz, 1H), 8.17 (s, 0.6H, FA), 7.88 (s, 1H), 7.59 (dd, J = |
| | | 9.7, 7.0 Hz, 2H), 6.76 (d, J = 7.3 Hz, 3H), 6.61 (d, J = 8.4 Hz, 1H), |
| | | 5.05 (dd, J = 12.8, 5.4 Hz, 1H), 4.87 (t, J = 5.4 Hz, 1H), 4.15-3.95 |
| | | (m, 2H), 3.84 (s, 6H), 3.67 (d, J = 15.2 Hz, 3H), 3.60 (s, 3H), 3.11- |
| | | 2.71 (m, 2H), 2.66-2.55 (m, 5H), 2.27 (s, 3H), 2.12-1.88 (m, |
| | | 4H), 1.75 (d, J = 10.1 Hz, 1H), 1.64-1.36 (m, 4H). |
| D228 | 791.55 | |
| D229 | 751.2 | |
| D230 | 791.4 | ¹H NMR (300 MHz, DMSO-d6) δ 11.12 (s, 1H), 9.45 (t, J = 1.2 Hz, |
| | | 1H), 8.73 (dd, J = 5.7, 1.0 Hz, 1H), 8.19 (s, 0.3H, FA), 7.99-7.73 |
| | | (m, 2H), 7.66-7.50 (m, 1H), 7.39-7.22 (m, 2H), 6.77 (d, J = 9.5 |
| | | Hz, 2H), 5.12 (dd, J = 12.9, 5.4 Hz, 1H), 4.87 (t, J = 6.8 Hz, 1H), |
| | | 4.56 (d, J = 19.1 Hz, 2H), 3.82 (d, J = 13.1 Hz, 6H), 3.60 (d, J = 1.5 |
| | | Hz, 3H), 3.26 (s, 2H), 3.17 (s, 2H), 2.89 (s, 1H), 2.78-2.61 (m, |
| | | 6H), 2.61-2.52 (m, 2H), 2.48-2.33 (m, 2H), 2.28 (dd, J = 3.8, 1.9 |
| | | Hz, 1H), 2.19 (dd, J = 11.7, 8.0 Hz, 2H), 2.04 (d, J = 11.6 Hz, 1H), |
| | | 1.53 (d, J = 7.9 Hz, 2H), 1.42-1.19 (m, 2H). |
| D231 | 774.2 | |
| D232 | 774.4 | |
| D233 | 735.2 | ¹H NMR (300 MHz, DMSO-d6) δ 11.13 (s, 1H), 9.45 (d, J = 0.8 Hz, |
| | | 1H), 8.72 (d, J = 5.7 Hz, 1H), 8.18 (s, 0.5H, FA), 7.93-7.79 (m, |
| | | 2H), 7.56 (dd, J = 5.7, 0.9 Hz, 1H), 7.31 (d, J = 7.8 Hz, 2H), 6.74 (s, |
| | | 2H), 5.27 (s, 1H), 5.14 (dd, J = 12.9, 5.3 Hz, 1H), 4.63 (t, J = 8.1 |
| | | Hz, 1H), 4.34 (dd, J = 10.5, 6.5 Hz, 1H), 4.13 (d, J = 8.3 Hz, 1H), |
| | | 3.82 (s, 7H), 3.73 (s, 2H), 3.60 (s, 3H), 3.50 (d, J = 9.7 Hz, 2H), |
| | | 3.07 (s, 2H), 2.98-2.80 (m, 1H), 2.71-2.53 (m, 3H), 2.38 (d, J = |
| | | 7.5 Hz, 2H), 2.17-1.97 (m, 1H). |
| D234 | 775.35 | ¹H NMR (300 MHz, DMSO-d6) δ 11.12 (s, 1H), 9.45 (s, 1H), 8.73 |
| | | (dd, J = 5.7, 1.2 Hz, 1H), 8.20 (s, 1H, FA), 7.96-7.76 (m, 2H), |
| | | 7.69-7.54 (m, 1H), 7.42-7.19 (m, 2H), 6.75 (d, J = 1.7 Hz, 2H), |
| | | 5.12 (dd, J = 12.9, 5.3 Hz, 1H), 4.90 (t, J = 6.7 Hz, 1H), 4.14 (d, J = |
| | | 27.9 Hz, 2H), 3.92 (s, 1H), 3.83 (d, J = 2.2 Hz, 7H), 3.75 (s, 2H), |
| | | 3.61 (s, 3H), 3.49 (t, J = 6.8 Hz, 3H), 3.08 (s, 2H), 2.99-2.70 (m, |
| | | 4H), 2.68-2.55 (m, 3H), 2.40-2.19 (m, 4H), 2.15-1.94 (m, 1H). |
| D235 | 729.3 | |
| D236 | 715.15 | |
| D237 | 689.2 | |
| D238 | 743.4 | |
| D239 | 729.35 | |
| D240 | 757.35 | |
| D241 | 729.15 | |
| D242 | 729.2 | |
| D243 | 757.35 | |
| D244 | 791.23 | |
| D245 | 762.4 | |
| D246 | 791.4 | |
| D247 | 790.4 | |
| D248 | 762.3 | |
| D249 | 723.3 | |
| D250 | 762.4 | |
| D251 | 763.6 | ¹H NMR (400 MHz, DMSO-d6) δ 11.12 (s, 1H), 9.45 (s, 1H), 8.73 |
| | | (d, J = 5.7 Hz, 1H), 8.21 (s, 1.4H, FA), 7.88 (s, 1H), 7.82 (d, J = 8.2 |
| | | Hz, 1H), 7.57 (d, J = 5.6 Hz, 1H), 7.28 (d, J = 2.2 Hz, 1H), 7.24 (dd, |
| | | J = 8.3, 2.3 Hz, 1H), 6.75 (s, 2H), 5.12 (dd, J = 12.8, 5.4 Hz, 1H), |
| | | 4.90-4.80 (m, 1H), 3.82 (s, 6H), 3.61 (d, J = 3.2 Hz, 5H), 3.36 (s, |
| | | 2H), 3.27 (s, 2H), 2.89 (ddd, J = 16.7, 13.7, 5.3 Hz, 1H), 2.75- |
| | | 2.56 (m, 4H), 2.45 (q, J = 7.1, 6.7 Hz, 4H), 2.26-2.13 (m, 5H), |
| | | 2.11-1.98 (m, 1H), 1.50 (t, J = 7.2 Hz, 2H), 1.32 (t, J = 7.2 Hz, 2H). |
| D252 | 762.4 | |
| D253 | 777.35 | |
| D254 | 748.4 | |
| D255 | 790.25 | |
| D256 | 818.2 | |
| D257 | 777.7 | |
| D258 | 790.4 | |
| D259 | 777.2 | |
| D260 | 805.35 | |
| D261 | 819.2 | |
| D262 | 819.25 | |
| D263 | 805.35 | |
| D264 | 803.2 | |
| D265 | 803.15 | |
| D266 | 789.3 | |
| D267 | 789.3 | |
| D268 | 715.3 | |
| D269 | 757.35 | |
| D270 | 719.35 | |
| D271 | 719.28 | ¹H NMR (400 MHz, DMSO-d6) δ 11.03 (s, 1H), 9.42 (s, 1H), 8.68 |
| | | (d, J = 5.6 Hz, 1H), 7.91 (s, 1H), 7.85 (s, 1H), 7.61 (d, J = 8.3 Hz, |
| | | 1H), 7.55 (d, J = 5.7 Hz, 1H), 6.80 (s, 2H), 6.75 (d, J = 2.1 Hz, 1H), |
| | | 6.62 (dd, J = 8.4, 2.1 Hz, 1H), 5.53 (s, 2H), 5.02 (dd, J = 12.8, 5.4 |
| | | Hz, 1H), 4.57 (td, J = 6.3, 3.2 Hz, 1H), 4.53 (s, 2H), 4.24-4.15 (m, |
| | | 2H), 3.86 (s, 6H), 3.79 (dd, J = 9.7, 3.9 Hz, 2H), 3.56 (s, 3H), 3.15 |
| | | (d, J = 5.3 Hz, 1H), 2.85 (ddd, J = 16.8, 13.8, 5.3 Hz, 1H), 2.60- |
| | | 2.50 (m, 2H), 2.05 (s, 1H), 2.03-1.94 (m, 1H). |
| D272 | 747.28 | |
| D273 | 720.03 | |
| D274 | 735.52 | |
| D275 | 765.06 | |
| D276 | 776.47 | |
| D277 | 776.33 | |
| D278 | 804.19 | |
| D279 | 761.28 | ¹H NMR (400 MHz, DMSO-d₆) δ 11.08 (s, 1H), 9.43 (s, 1H), 8.71 |
| | | (d, J = 5.6 Hz, 1H), 8.05 (s, 1H), 7.86 (s, 1H), 7.82 (d, J = 8.3 Hz, |
| | | 1H), 7.56 (d, J = 5.7 Hz, 1H), 7.45 (d, J = 2.3 Hz, 1H), 7.35 (dd, J = |
| | | 8.4, 2.3 Hz, 1H), 6.76 (s, 2H), 5.09 (dd, J = 12.9, 5.4 Hz, 1H), 4.41 |
| | | (t, J = 6.6 Hz, 2H), 3.83 (s, 5H), 3.59 (s, 2H), 3.15 (d, J = 5.1 Hz, |
| | | 1H), 3.11 (d, J = 6.4 Hz, 1H), 2.87 (ddd, J = 17.2, 13.9, 5.3 Hz, 1H), |
| | | 2.70-2.51 (m, 2H), 2.03 (d, J = 15.9 Hz, 5H). |
| D280 | 802.16 | |
| D281 | 830.16 | |
| D282 | 735.45 | ¹H NMR (400 MHz, DMSO-d6) δ 11.08 (s, 1H), 7.97 (s, 1H), 7.86 |
| | | (s, 1H), 7.75 (d, J = 8.2 Hz, 1H), 7.47 (d, J = 2.1 Hz, 1H), 7.32 (dd, |
| | | J = 8.3, 2.2 Hz, 1H), 6.71 (s, 2H), 5.26 (s, 2H), 4.40 (s, 1H), 3.78 |
| | | (s, 5H), 3.55 (s, 3H), 2.88 (ddd, J = 18.2, 13.8, 5.4 Hz, 1H), 2.71- |
| | | 2.53 (m, 2H), 2.38-2.24 (m, 2H), 2.09 (d, J = 28.1 Hz, 4H). |
| D283 | 749.31 | |
| D284 | 779.27 | |
| D285 | 790.33 | |
| D286 | 790.4 | ¹H NMR (400 MHz, DMSO-d6) δ 11.03 (s, 1H), 8.08 (s, 1H), 7.85 |
| | | (s, 1H), 7.60 (d, J = 8.3 Hz, 1H), 6.72 (s, 2H), 5.03 (dd, J = 12.9, |
| | | 5.4 Hz, 1H), 4.55 (s, 2H), 4.20 (dd, J = 9.2, 6.3 Hz, 2H), 3.80 (s, |
| | | 6H), 3.58 (s, 2H), 2.97-2.72 (m, 0H), 2.18 (s, 1H), 2.05 (s, 1H), |
| | | 2.02-1.94 (m, 1H). |
| D287 | 818.26 | |
| D288 | 765.27 | |
| D289 | 747.35 | |
| D290 | 791.24 | |
| D291 | 802.37 | |
| D292 | 779.2 | |
| D293 | 809.16 | |
| D294 | 820.29 | |
| D295 | 820.08 | |
| D296 | 847.22 | |
| D297 | 719.28 | |
| D298 | 733.49 | |
| D299 | 763.31 | |
| D300 | 774.44 | |
| D301 | 774.02 | |
| D302 | 802.58 | |
| D303 | 708.22 | |
| D304 | 803.4 | ¹H NMR (400 MHz, Methanol-d4) δ 9.58 (s, 1H), 8.70 (d, J = 6.0 |
| | | Hz, 1H), 7.91 (d, J = 2.2 Hz, 1H), 7.82 (d, J = 8.3 Hz, 1H), 7.78 (d, |
| | | J = 6.1 Hz, 1H), 7.31 (d, J = 2.3 Hz, 1H), 7.26 (dd, J = 8.3, 2.3 Hz, |
| | | 1H), 6.89 (s, 2H), 5.13 (dd, J = 12.6, 5.4 Hz, 1H), 4.98 (t, J = 6.5 |
| | | Hz, 1H), 4.43 (s, 2H), 3.98 (d, J = 4.3 Hz, 6H), 3.74 (s, 3H), 3.70- |
| | | 3.50 (m, 4H), 3.33-2.94 (m, 6H), 2.93-2.66 (m, 4H), 2.56 (s, |
| | | 1H), 2.27 (s, 1H), 2.17-1.95 (m, 10H), 1.67 (q, J = 12.6 Hz, 1H). |
| D305 | 789.7 | ¹H NMR (400 MHz, Methanol-d4) δ 9.54 (s, 1H), 8.69 (d, J = 5.8 |
| | | Hz, 1H), 8.50 (s, 2H, FA), 7.83 (d, J = 8.3 Hz, 1H), 7.75 (s, 1H), |
| | | 7.62 (d, J = 5.7 Hz, 1H), 7.31 (d, J = 2.2 Hz, 1H), 7.26 (dd, J = 8.3, |
| | | 2.2 Hz, 1H), 6.82 (s, 2H), 5.13 (dd, J = 12.5, 5.4 Hz, 1H), 5.01- |
| | | 4.97 (m, 1H), 4.17 (s, 2H), 3.95 (s, 6H), 3.77-3.65 (m, 5H), 3.56- |
| | | 3.40 (m, 5H), 3.28 (s, 1H), 3.07-2.92 (m, 3H), 2.91-2.84 (m, |
| | | 1H), 2.81-2.65 (m, 4H), 2.50-2.40 (m, 1H), 2.18-2.07 (m, 6H), |
| | | 2.05-1.96 (m, 2H). |
| D306 | 715.3 | ¹H NMR (400 MHz, DMSO-d6) δ 11.15 (s, 1H), 9.45 (s, 1H), 8.72 |
| | | (d, J = 5.6 Hz, 1H), 8.18 (s, 1H FA), 7.89 (d, J = 17.4 Hz, 4H), 7.56 |
| | | (d, J = 5.6 Hz, 1H), 6.74 (s, 2H), 5.17 (dd, J = 12.8, 5.4 Hz, 1H), |
| | | 3.82 (s, 6H), 3.74 (s, 2H), 3.63 (d, J = 19.3 Hz, 6H), 3.27 (s, 3H), |
| | | 2.90 (ddd, J = 16.8, 13.7, 5.3 Hz, 1H), 2.78 (s, 2H), 2.66-2.57 (m, |
| | | 3H), 2.55 (s, 1H), 2.11-2.02 (m, 1H), 1.96 (t, J = 6.9 Hz, 2H). |
| D307 | 729.3 | ¹H NMR (400 MHz, DMSO-d6) δ 11.15 (s, 1H), 9.45 (s, 1H), 8.72 |
| | | (d, J = 5.7 Hz, 1H), 7.99-7.80 (m, 4H), 7.56 (d, J = 5.7 Hz, 1H), |
| | | 6.73 (s, 2H), 5.16 (dd, J = 12.7, 5.4 Hz, 1H), 3.82 (s, 6H), 3.71 (s, |
| | | 2H), 3.60 (s, 3H), 3.53 (s, 2H), 3.10 (s, 4H), 2.90 (ddd, J = 16.7, |
| | | 13.6, 5.4 Hz, 1H), 2.65-2.54 (m, 1H), 2.44 (s, 5H), 2.12-2.01 (m, |
| | | 1H), 1.67 (t, J = 5.5 Hz, 4H). |
| D308 | 743.35 | |
| D309 | 701.3 | |
| D310 | 743.55 | |
| D311 | 743.3 | |
| D312 | 757.3 | |
| D313 | 771.45 | |
| D314 | 743.3 | |
| D315 | 743.3 | |
| D316 | 717.3 | |
| D317 | 729.3 | |
| D318 | 757.3 | |
| D319 | 761.35 | |
| D320 | 761.28 | |
| D321 | 763.24 | |
| D322 | 747.42 | |
| D323 | 746.83 | |
| D324 | 746.55 | |
| D325 | 747.33 | |
| D326 | 747.45 | |
| D327 | 706.67 | |
| D328 | 779.84 | ¹H NMR (400 MHz, DMSO-d6) δ 11.04 (s, 1H), 8.48 (d, J = 2.7 Hz, |
| | | 1H), 8.25 (d, J = 2.7 Hz, 1H), 8.19 (s, 2H), 7.65 (d, J = 8.5 Hz, 1H), |
| | | 7.30 (d, J = 2.3 Hz, 1H), 7.22 (dd, J = 8.6, 2.3 Hz, 1H), 6.85 (d, J = |
| | | 5.6 Hz, 2H), 5.04 (dd, J = 12.9, 5.4 Hz, 1H), 3.83 (d, J = 2.7 Hz, |
| | | 7H), 3.59 (s, 3H), 3.48 (d, J = 5.0 Hz, 2H), 3.39 (t, J = 5.0 Hz, 4H), |
| | | 2.81 (dd, J = 25.4, 11.4 Hz, 3H), 2.63-2.51 (m, 2H), 2.32-2.22 |
| | | (m, 2H), 2.06-1.90 (m, 1H), 1.56 (s, 1H), 1.34 (d, J = 7.5 Hz, 2H), |
| | | 1.09 (s, 1H) |
| D329 | 725.87 | ¹H NMR (400 MHz, DMSO-d6) δ 11.04 (s, 1H), 8.24-8.12 (m, |
| | | 2H), 8.03 (d, J = 2.6 Hz, 1H), 7.88-7.72 (m, 1H), 7.65 (d, J = 8.5 |
| | | Hz, 1H), 7.30 (d, J = 2.2 Hz, 1H), 7.22 (dd, J = 8.6, 2.3 Hz, 1H), |
| | | 6.79 (s, 2H), 3.83 (s, 6H), 3.57 (s, 2H), 3.51 (s, 3H), 3.40 (t, J = 5.1 |
| | | Hz, 4H), 2.91-2.78 (m, 3H), 2.66-2.50 (m, 2H), 2.36-2.24 (m, |
| | | 2H), 2.14 (t, J = 11.6 Hz, 2H), 2.08 (s, 3H), 1.99 (ddd, J = 11.5, 6.0, |
| | | 3.7 Hz, 1H), 1.61 (d, J = 12.4 Hz, 2H), 1.35 (q, J = 7.0 Hz, 2H), |
| | | 1.26 (s, 2H), 1.13 (q, J = 11.2, 10.7 Hz, 2H). |
| D330 | 614.68 | ¹H NMR (400 MHz, DMSO-d6) δ 11.04 (s, 1H), 8.15 (s, 1H), 8.02 |
| | | (d, J = 2.7 Hz, 1H), 7.79 (dd, J = 2.8, 1.3 Hz, 1H), 7.63 (d, J = 8.5 |
| | | Hz, 1H), 7.27 (d, J = 2.3 Hz, 1H), 7.20 (dd, J = 8.7, 2.3 Hz, 1H), |
| | | 6.80 (s, 2H), 5.04 (dd, J = 12.9, 5.4 Hz, 1H), 3.84 (s, 6H), 3.55 (s, |
| | | 2H), 3.37 (t, J = 5.1 Hz, 4H), 2.66-2.53 (m, 2H), 2.08 (s, 3H). |
| D331 | 654.74 | ¹H NMR (400 MHz, DMSO-d6) δ 11.03 (s, 1H), 8.12 (s, 1H), 8.04 |
| | | (d, J = 2.6 Hz, 1H), 7.80 (dd, J = 2.7, 1.3 Hz, 1H), 7.61 (d, J = 8.3 |
| | | Hz, 1H), 6.82 (s, 2H), 6.75 (d, J = 2.1 Hz, 1H), 5.02 (dd, J = 12.9, |
| | | 5.4 Hz, 1H), 3.85 (s, 6H), 3.72 (s, 5H), 3.52 (s, 3H), 2.93-2.74 (m, |
| | | 1H), 2.08 (s, 3H), 1.98 (dd, J = 9.2, 4.2 Hz, 1H), 1.76 (s, 5H). |
| D332 | 669.75 | |
| D333 | 724.79 | |
| D334 | 594.73 | ¹H NMR (400 MHz, DMSO-d6) δ 10.81 (s, 1H), 8.13 (s, 1H), 8.05 |
| | | (d, J = 2.7 Hz, 1H), 7.80 (dd, J = 2.8, 1.3 Hz, 1H), 6.82 (s, 2H), 5.73 |
| | | (s, 1H), 3.85 (s, 6H), 3.71 (s, 2H), 3.52 (s, 3H), 3.08-2.85 (m, 4H), |
| | | 2.79-2.53 (m, 3H), 2.38-2.28 (m, 3H), 2.08 (s, 3H), 1.86-1.74 |
| | | (m, 1H), 1.65 (d, J = 12.7 Hz, 2H), 1.33 (s, 3H), 1.27-1.12 (m, 3H). |
| D335 | 609.66 | |
| D336 | 654.74 | |
| D337 | 640.72 | |
| D338 | 640.72 | |
| D339 | 626.69 | |
| D340 | 679.75 | ¹H NMR (400 MHz, DMSO-d6) δ 12.12 (s, 1H), 11.03 (s, 1H), 8.14 |
| | | (d, J = 1.1 Hz, 1H), 7.60 (d, J = 8.3 Hz, 1H), 7.45 (s, 1H), 7.34 (t, J = |
| | | 2.8 Hz, 1H), 6.82 (s, 2H), 6.75 (d, J = 2.1 Hz, 1H), 6.62 (dd, J = |
| | | 8.4, 2.2 Hz, 1H), 6.54 (t, J = 2.4 Hz, 1H), 5.73 (s, 1H), 5.02 (dd, J = |
| | | 12.9, 5.4 Hz, 1H), 3.83 (s, 6H), 3.71 (s, 4H), 3.58 (s, 3H), 3.53 (s, |
| | | 2H), 2.86 (ddd, J = 17.3, 13.9, 5.4 Hz, 1H), 2.64-2.50 (m, 1H), |
| | | 1.98 (dd, J = 9.2, 4.0 Hz, 1H), 1.72 (d, J = 5.8 Hz, 4H). |
| D341 | 690.72 | ¹H NMR (400 MHz, DMSO-d6) δ 8.13 (dd, J = 9.6, 2.7 Hz, 2H), |
| | | 6.83 (d, J = 0.9 Hz, 2H), 11.03 (s, 1H), 8.37 (d, J = 2.6 Hz, 1H), |
| | | 7.60 (d, J = 8.3 Hz, 1H), 6.78-6.71 (m, 1H), 6.62 (dd, J = 8.4, 2.1 |
| | | Hz, 1H), 5.02 (dd, J = 12.9, 5.4 Hz, 1H), 3.84 (d, J = 0.8 Hz, 6H), |
| | | 3.69 (s, 4H), 3.57 (s, 3H), 3.50 (d, J = 4.1 Hz, 2H), 2.86 (ddd, J = |
| | | 17.3, 13.9, 5.4 Hz, 1H), 2.38 (s, 5H), 1.69 (s, 4H). |
| D342 | 712.15 | ¹H NMR (400 MHz, DMSO-d6) δ 11.07 (s, 1H), 9.04 (d, J = 3.3 Hz, |
| | | 1H), 8.66 (d, J = 3.4 Hz, 1H), 8.20 (s, 1H, FA), 7.64 (d, J = 8.5 Hz, |
| | | 1H), 7.29 (s, 1H), 7.22 (d, J = 8.8 Hz, 1H), 6.88 (s, 2H), 5.06 (dd, J = |
| | | 13.0, 5.3 Hz, 1H), 4.02 (d, J = 12.8 Hz, 2H), 3.84 (s, 6H), 3.57- |
| | | 3.47 (m, 5H), 2.91 (dt, J = 22.4, 13.1 Hz, 3H), 2.71-2.55 (m, 2H), |
| | | 2.42-2.23 (m, 10H), 2.09-1.93 (m, 1H), 1.82-1.68 (m, 2H), |
| | | 1.64-1.50 (m, 1H), 1.39-1.30 (m, 2H), 1.22-1.09 (m, 2H). |
| D343 | 628.5 | ¹H NMR (300 MHz, DMSO-d6) δ 11.10 (s, 1H), 9.47 (s, 1H, TFA), |
| | | 7.77 (d, J = 8.5 Hz, 1H), 7.47 (d, J = 2.2 Hz, 1H), 7.38-7.23 (m, |
| | | 2H), 6.69 (s, 2H), 5.10 (dd, J = 12.8, 5.4 Hz, 1H), 4.33 (s, 2H), 4.18 |
| | | (d, J = 12.1 Hz, 2H), 3.89 (s, 6H), 3.55 (s, 4H), 3.53-3.45 (m, 5H), |
| | | 2.99-2.81 (m, 1H), 2.60 (d, J = 18.3 Hz, 2H), 2.35 (s, 3H), 2.05 (s, |
| | | 4H). |
| D344 | 600.2 | ¹H NMR (400 MHz, DMSO-d6) δ 11.07 (s, 1H), 8.21 (d, J = 2.8 Hz, |
| | | 1H), 8.14 (s, 1H FA), 7.97-7.88 (m, 1H), 7.67 (d, J = 8.5 Hz, 1H), |
| | | 7.32 (d, J = 2.3 Hz, 1H), 7.26-7.21 (m, 1H), 6.85 (s, 2H), 6.50 (d, |
| | | J = 9.4 Hz, 1H), 5.10-5.00 (m, 1H), 3.87 (s, 6H), 3.67 (s, 2H), |
| | | 3.53 (s, 3H), 3.44 (d, 5H). 2.97-2.78 (m, 1H), 2.67-2.60 (m, 5H), |
| | | 2.58-2.52 (m, 1H), 2.09-1.92 (m, 1H). |
| D345 | 737.3 | ¹H NMR (300 MHz, Methanol-d4) δ 8.31 (s, 1H FA), 7.65 (d, J = 8.3 |
| | | Hz, 1H), 7.48 (s, 1H), 6.84 (d, J = 2.1 Hz, 1H), 6.72-6.63 (m, 3H), |
| | | 5.07 (dd, J = 12.4, 5.4 Hz, 1H), 4.48 (s, 2H), 4.25 (s, 2H), 4.06- |
| | | 3.90 (m, 8H), 3.82 (s, 4H), 3.58 (d, J = 20.8 Hz, 4H), 2.97-2.66 |
| | | (m, 5H), 2.63 (s, 3H), 2.27-2.03 (m, 8H), 1.95 (s, 4H). |
| D346 | 737.7 | ¹H NMR (300 MHz, DMSO-d6) δ 11.07 (s, 1H), 8.16(s, 1H, FA), |
| | | 7.64 (d, J = 8.3 Hz, 1H), 7.28 (d, J = 1.2 Hz, 1H), 6.77 (d, J = 2.1 |
| | | Hz, 1H), 6.69-6.53 (m, 3H), 5.05 (dd, J = 12.7, 5.4 Hz, 1H), 3.92- |
| | | 3.85 (m, 2H), 3.82 (s, 6H), 3.74 (s, 4H), 3.71-3.61 (m, 2H), 3.54 |
| | | (s, 4H), 2.98-2.78 (m, 2H), 2.71-2.54 (m, 2H), 2.54-2.50 (m, |
| | | 2H), 2.48-2.42 (m, 3H), 2.37-2.20 (m, 4H), 2.11-1.93 (m, 4H), |
| | | 1.82-1.65 (m, 4H), 1.20 (d, J = 26.6 Hz, 1H). |
| D347 | 709.2 | ¹H NMR (400 MHz, DMSO-d6) δ 11.08 (s, 1H), 8.23 (s, 1H), 8.17 |
| | | (s, 1H, FA), 7.94 (d, J = 9.6 Hz, 1H), 7.64 (d, J = 8.3 Hz, 1H), 6.86 |
| | | (d, J = 4.5 Hz, 2H), 6.77 (d, J = 2.1 Hz, 1H), 6.64 (dd, J = 8.4, 2.2 |
| | | Hz, 1H), 6.50 (d, J = 9.4 Hz, 1H), 5.05 (dd, J = 12.9, 5.3 Hz, 1H), |
| | | 3.88 (t, J = 2.1 Hz, 7H), 3.79 (s, 2H), 3.73 (s, 5H), 3.54 (s, 6H), |
| | | 3.19 (d, J = 29.3 Hz, 1H), 2.99-2.81 (m, 1H), 2.58 (d, J = 16.2 Hz, |
| | | 2H), 2.44 (s, 2H), 2.28 (s, 3H), 2.01 (d, J = 12.4 Hz, 1H), 1.73 (s, 4H). |
| D348 | 749.25 | ¹H NMR (300 MHz, DMSO-d6) δ 8.04 (d, J = 2.6 Hz, 1H), 7.88 (t, J = |
| | | 1.8 Hz, 1H), 7.67 (d, J = 8.2 Hz, 1H), 6.90 (d, J = 2.1 Hz, 2H), |
| | | 6.77 (d, J = 2.2 Hz, 1H), 6.66 (m, J = 8.3, 2.0 Hz, 1H), 6.09-5.91 |
| | | (m, 1H), , 5.19 (m, J = 10.3, 1.5 Hz, 1H), 5.14-4.98 (m, 2H), 4.62 |
| | | (d, J = 5.4 Hz, 2H), 4.34 (d, J = 16.5 Hz, 2H), 4.18 (s, 2H), 3.99 (d, |
| | | J = 10.2 Hz, 2H), 3.92 (s, 6H), 3.87 (s, 2H), 3.81 (s, 2H), 3.41 (d, J = |
| | | 6.6 Hz, 4H), 3.17 (d, J = 8.1 Hz, 1H), 2.94 (s, 3H), 2.89-2.78 (m, |
| | | 1H), 2.65-2.54 (m, 1H), 2.40-2.23 (m, 1H), 2.11 (s, 4H), 2.06- |
| | | 1.83 (m, 3H). |
| D349 | 723.2 | ¹H NMR (400 MHz, DMSO-d6) δ 11.08 (s, 1H), 9.78 (s, 2H, TFA), |
| | | 7.69 (d, J = 8.2 Hz, 1H), 7.65 (s, 1H), 6.75 (dd, J = 21.5, 3.2 Hz, |
| | | 3H), 6.66 (dd, J = 8.3, 2.4 Hz, 1H), 6.38 (s, 1H), 5.06 (dd, J = 12.9, |
| | | 5.4 Hz, 1H), 4.39 (s, 1H), 4.34 (d, J = 5.5 Hz, 1H), 4.22 (s, 2H), |
| | | 4.01 (d, J = 8.8 Hz, 2H), 3.89 (s, 8H), 3.82 (s, 2H), 3.46 (s, 5H), |
| | | 3.25-3.08 (m, 2H), 3.03-2.82 (m, 3H), 2.64-2.59 (m, 2H), 2.21- |
| | | 2.09 (m, 5H), 2.09-1.77 (m, 4H). |
| D350 | 795.4 | ¹H NMR (300 MHz, MeOD) δ 8.04 (d, 1H), 7.82 (d, 1H), 7.67 (d, |
| | | 1H), 6.95-6.84 (m, 3H), 6.71 (dd, 1H), 5.08 (dd, 1H), 4.58-4.45 |
| | | (m, 2H), 4.34 (t, 2H), 4.24 (s, 2H), 4.17-4.09 (m, 2H), 4.01 (s, 6H), |
| | | 3.94-3.86 (m, 4H), 3.69 (s, 3H), 3.55-3.49 (m, 5H), 3.20-3.03 |
| | | (m, 2H), 2.91-2.77 (m, 2H), 2.72 (s, 4H), 2.35-2.00 (m, 5H). |
| D351 | 748.7 | ¹H NMR (400 MHz, DMSO-d6) δ 11.08 (s, 1H), 9.94 (br s, 2H, TFA |
| | | salt), 8.07 (s, 1H), 7.69 (d, J = 8.2 Hz, 1H), 6.77 (d, J = 3.7 Hz, 3H), |
| | | 6.66 (dd, J = 8.4, 2.3 Hz, 1H), 5.06 (dd, J = 12.8, 5.4 Hz, 1H), 4.40 |
| | | (s, 1H), 4.35 (d, J = 5.6 Hz, 1H), 4.27-4.16 (m, 2H), 4.03 (q, J = |
| | | 8.7, 7.5 Hz, 2H), 3.88 (s, 8H), 3.82 (s, 2H), 3.55 (s, 3H), 3.39 (s, |
| | | 5H), 3.18 (s, 1H), 3.04-2.82 (m, 3H), 2.64-2.54 (m, 2H), 2.36 (s, |
| | | 3H), 2.15 (d, J = 14.0 Hz, 2H), 2.02 (dd, J = 9.7, 4.6 Hz, 1H), 1.97- |
| | | 1.84 (m, 2H). |
| D352 | 734.45 | ¹H NMR (400 MHz, DMSO-d6) δ 11.07 (s, 1H), 8.77 (s, 1H), 8.69 |
| | | (s, 1H), 8.14 (s, 0.4H, FA), 7.67 (d, J = 8.3 Hz, 1H), 7.02 (s, 2H), |
| | | 6.77 (s, 1H), 6.65 (d, J = 8.4 Hz, 1H), 5.05 (dd, J = 12.6, 5.4 Hz, |
| | | 1H), 4.30 (s, 2H), 4.14 (s, 3H), 3.95 (s, 7H), 3.91-3.78 (m, 6H), |
| | | 3.63 (s, 4H), 2.96-2.80 (m, 2H), 2.97-2.79 (m, 5H), 2.05-1.79 |
| | | (m, 5H). |
| D353 | 723.5 | ¹H NMR (400 MHz, DMSO-d6) δ 11.08 (s, 1H), 8.16 (s, 1H FA), |
| | | 7.72 (d, J = 2.5 Hz, 1H), 7.64 (d, J = 8.3 Hz, 1H), 7.52 (dd, J = 2.7, |
| | | 1.2 Hz, 1H), 6.99 (s, 1H), 6.88 (s, 1H), 6.78 (d, J = 2.1 Hz, 1H), |
| | | 6.65 (dd, J = 8.5, 2.1 Hz, 1H), 5.05 (dd, J = 12.9, 5.4 Hz, 1H), 3.78 |
| | | (s, 3H), 3.74 (d, J = 2.8 Hz, 7H), 3.60 (s, 2H), 3.49 (s, 6H), 2.90 (s, |
| | | 3H), 2.73-2.58 (m, 5H), 2.39-2.19 (m, 3H), 2.05 (s, 3H), 2.02 (d, |
| | | J = 7.1 Hz, 1H), 1.74 (s, 4H). |
| D354 | 737.45 | ¹H NMR (400 MHz, Methanol-d4) δ 8.30 (s, 2H FA), 7.64 (d, J = 8.3 |
| | | Hz, 1H), 7.35 (s, 1H), 7.15 (s, 1H), 6.95 (s, 1H), 6.83 (d, J = 2.0 Hz, |
| | | 1H), 6.67 (dd, J = 8.3, 2.0 Hz, 1H), 5.07 (dd, J = 12.4, 5.5 Hz, 1H), |
| | | 4.43 (s, 2H), 4.26 (s, 2H), 4.03 (s, 2H), 3.91 (s, 3H), 3.83 (s, 4H), |
| | | 3.79 (s, 3H), 3.60 (s, 3H), 3.29 (s, 1H), 3.03 (s, 2H), 2.95-2.64 (m, |
| | | 7H), 2.16 (s, 3H), 2.15-2.07 (m, 1H), 2.07-1.87 (m, 7H). |
| D355 | 809.5 | ¹H NMR (300 MHz, MeOD) δ 8.07 (d, 1H), 7.74-7.63 (m, 2H), |
| | | 6.88 (d, 3H), 6.71 (dd, 1H), 5.08 (dd, 1H), 4.58-4.45 (m, 2H), 4.41- |
| | | 4.28 (m, 4H), 4.19-4.07 (m, 2H), 4.01 (s, 6H), 3.98-3.82 (m, |
| | | 4H), 3.70 (s, 3H), 3.58-3.41 (m, 5H), 3.18-3.02(m, 2H), 2.98 (s, |
| | | 3H), 2.93-2.79 (m, 2H), 2.76 (s, 3H), 2.77-2.66 (m, 1H), 2.40- |
| | | 2.01 (m, 5H). |
| D356 | 745.5 | ¹H NMR (300 MHz, DMSO-d6) δ 10.98 (s, 1H), 9.93 (br s, 2H, TFA |
| | | salt), 8.36 (d, J = 8.0 Hz, 1H), 7.78-7.53 (m, 4H), 7.41 (d, J = 8.5 |
| | | Hz, 1H), 6.85 (s, 2H), 6.70 (s, 2H), 5.07 (dd, J = 13.2, 4.9 Hz, 1H), |
| | | 4.50-3.96 (m, 8H), 3.90 (s, 6H), 3.78-3.55 (m, 8H), 3.53-3.49 |
| | | (m, 1H), 3.28-3.12 (m, 2H), 3.09-2.82 (m, 3H), 2.75-2.56 (m, |
| | | 1H), 2.43-2.24 (m, 2H), 2.19-1.83 (m, 5H) |
| D357 | 641.748028 | |
| D358 | 641.748028 | |
| D359 | 737.4 | ¹H NMR (300 MHz, DMSO-d6) δ 11.10 (s, 1H), 10.32-9.43 (m, |
| | | 2H), 7.69 (d, J = 8.3 Hz, 1H), 7.22-7.05 (m, 2H), 6.92 (s, 1H), |
| | | 6.83-6.74 (m, 1H), 6.66 (dd, J = 8.2, 2.1 Hz, 1H), 5.17-4.99 (m, |
| | | 1H), 4.57-4.33 (m, 2H), 4.34-4.15 (m, 2H), 4.15-3.94 (m, 2H), |
| | | 3.90 (s, 2H), 3.82 (s, 6H), 3.73 (m, 3H), 3.52 (s, 4H), 3.25-3.10 |
| | | (m, 2H), 3.08-2.79 (m, 4H), 2.62 (m, 2H), 2.59-2.54 (m, 1H), |
| | | 2.54-2.41 (m, 1H), 2.23-2.06 (m, 3H), 2.02 (s, 4H), 2.00-1.83 |
| | | (m, 2H). |
| D360 | 745.6 | ¹H NMR (300 MHz, DMSO-d6) δ 10.95 (s, 1H), 9.83 (br s, 2H, TFA |
| | | salt), 8.36 (d, J = 7.9 Hz, 1H), 7.73 (t, J = 7.8 Hz, 1H), 7.66-7.48 |
| | | (m, 4H), 6.85 (s, 2H), 6.55-6.44 (m, 2H), 5.04 (dd, J = 13.2, 4.8 Hz, |
| | | 1H), 4.40 (t, J = 13.8 Hz, 2H), 4.32-4.13 (m, 4H), 4.05 (s, 2H), |
| | | 3.90 (s, 6H), 3.81-3.65 (m, 5H), 3.60 (s, 4H), 3.55-3.50 (m, 2H), |
| | | 3.20 (s, 1H), 3.10-2.80 (m, 3H), 2.62 (s, 1H), 2.41-2.24 (m, 1H), |
| | | 2.19-2.05 (m, 2H), 2.02-1.82 (m, 3H). |
| D361 | 735.4 | ¹H NMR (400 MHz, DMSO-d6) δ 8.33 (s, 1H), 8.14 (s, FA, 1H), |
| | | 7.91 (s, 1H), 7.40 (d, J = 8.9 Hz, 1H), 7.13 (s, 1H), 7.03 (s, 2H), |
| | | 6.73-6.67 (m, 2H), 5.06 (dd, J = 13.2, 5.1 Hz, 1H), 4.34 (s, J = |
| | | 16.7 Hz, 3H), 4.30 (d, J = 16.7 Hz, 1H) 4.19 (d, J = 16.7 Hz, 1H), |
| | | 4.14 (m, 2H), 3.92 (s, 6H), 3.90-3.80 (m, 2H), 3.63 (s, 4H), 3.06 (s, |
| | | 2H), 2.96-2.82 (m, 3H), 2.74-2.56 (m, 3H), 2.45-2.32 (m, 2H), |
| | | 2.01-1.92 (m, 2H), 1.87 (s, 5H). |
| D362 | 725.3 | ¹H NMR (400 MHz, DMSO-d6) δ 10.95 (s, 1H), 8.17 (s, 1H, FA), |
| | | 7.55-7.46 (m, 2H), 7.04 (s, 2H), 6.54 (s, 2H), 5.04 (dd, J = 13.3, |
| | | 5.1 Hz, 1H), 4.36-4.12 (m, 2H), 3.85 (d, J = 12.7 Hz, 2H), 3.77 (s, |
| | | 6H), 3.53 (s, 2H), 3.46 (s, 3H), 2.97-2.74 (m, 3H), 2.61 (s, 1H), |
| | | 2.47-2.28 (m, 11H), 2.06 (d, J = 2.7 Hz, 6H), 1.97 (s, 1H), 1.73 (d, |
| | | J = 12.6 Hz, 2H), 1.50 (s, 1H), 1.36 (d, J = 7.5 Hz, 2H), 1.26-1.13 |
| | | (m, 2H). |
| D363 | 735.6 | ¹H NMR (400 MHz, DMSO-d6) δ 10.94 (s, 1H), 8.33 (s, 1H), 8.22 |
| | | (s, FA, 1H), 7.89 (s, 1H), 7.48 (d, J = 8.3 Hz, 1H), 7.11 (s, 1H), 6.92 |
| | | (s, 2H), 6.53-6.43 (t, 2H), 5.03 (dd, J = 13.3, 5.1 Hz, 1H), 4.30 (d, |
| | | J = 16.9 Hz, 1H), 4.17 (d, J = 16.9 Hz, 1H), 3.85 (s, 6H), 3.67 (s, |
| | | 2H), 3.61 (s, J = 6.9 Hz, 6H), 3.47 (s, J = 6.9 Hz, 4H), 2.98 (m, J = |
| | | 6.9 Hz, 4H), 2.60 (s, 1H), 2.46-2.34 (m, 3H), 2.28 (s, 3H), 1.99- |
| | | 1.89 (m, 1H), 1.72 (t, J = 5.2 Hz, 4H). |
| D364 | 781.2 | ¹H NMR (300 MHz, MeOD) δ 8.05 (d, J = 2.5 Hz, 1H), 7.83 (s, 1H), |
| | | 7.41 (d, J = 8.2 Hz, 1H), 6.95-6.84 (m, 3H), 6.79 (d, J = 8.2 Hz, |
| | | 1H), 5.14 (dd, J = 13.2, 5.1 Hz, 1H), 4.51-1.45 (m, 2H), 4.44- |
| | | 4.30 (m, 4H), 4.25 (s, 2H), 4.14 (s, 2H), 4.01 (s, 6H), 3.79-3.73 |
| | | (m, 4H), 3.69 (s, 3H), 3.55-3.48 (m, 4H), 3.18-3.04 (m, 2H), 3.00- |
| | | 2.78 (m, 2H), 2.72 (s, 3H), 2.60-2.41 (m, 1H), 2.28-2.12 (m, |
| | | 5H), 1.38-1.28 (m, 2H). |
| D365 | 735.45 | ¹H NMR (400 MHz, MeOD) δ 8.48 (s, FA, 1H), 7.93 (d, J = 1.6 Hz, |
| | | 1H), 7.77 (s, 1H), 7.49 (d, J = 1.6 Hz, 1H), 7.39 (d, J = 8.2 Hz, 1H), |
| | | 7.25 (s, 2H), 6.85 (d, J = 2.2 Hz, 1H), 6.77 (dd, J = 8.3, 2.2 Hz, 1H), |
| | | 5.14 (dd, J = 13.3, 5.2 Hz, 1H), 4.48 (s, 2H), 4.45-4.33 (m, 2H), |
| | | 4.23 (s, 735.452H), 4.02 (s, 6H), 3.97 (s, 2H), 3.72 (s, 3H), 3.67 (s, |
| | | 4H), 3.43-3.35 (m, 1H), 3.22-3.01 (m, 1H), 2.96-2.85 (m, 1H), |
| | | 2.84-2.75 (m, 1H), 2.74 (s, 2H), 2.64-2.42 (m, 5H), 2.23-2.13 |
| | | (m, 1H), 1.91 (s, 4H). |
| D366 | 790.2 | ¹H NMR (400 MHz, DMSO-d6) δ 10.98 (s, 1H), 9.56 (d, J = 5.4 Hz, |
| | | 1H), 9.29 (s, 2H, TFA), 8.39 (d, J = 5.4 Hz, 1H), 8.08 (s, 1H), 7.64 |
| | | (dd, J = 8.4, 3.2 Hz, 1H), 7.17-7.04 (m, 3H), 7.02-6.95 (m, 1H), |
| | | 5.08 (dd, J = 13.3, 5.1 Hz, 1H), 4.88 (p, J = 6.7 Hz, 1H), 4.43- |
| | | 4.14 (m, 4H), 3.90 (s, 6H), 3.65 (s, 3H), 3.47-3.15 (m, 4H), 3.09- |
| | | 2.78 (m, 7H), 2.60 (d, J = 16.2 Hz, 2H), 2.46-2.34 (m, 2H), 2.17- |
| | | 2.08 (m, 1H), 1.98-1.87 (m, 6H), 1.86-1.82 (m, 3H), 1.49 (q, J = |
| | | 12.7 Hz, 2H). |
| D367 | 790.5 | ¹H NMR (400 MHz, DMSO-d6) δ 10.98 (s, 1H), 9.56 (d, J = 5.4 Hz, |
| | | 1H), 9.29 (s, 2H, TFA), 8.39 (d, J = 5.4 Hz, 1H), 8.08 (s, 1H), 7.64 |
| | | (dd, J = 8.4, 3.2 Hz, 1H), 7.17-7.04 (m, 3H), 7.02-6.95 (m, 1H), |
| | | 5.08 (dd, J = 13.3, 5.1 Hz, 1H), 4.88 (p, J = 6.7 Hz, 1H), 4.43- |
| | | 4.14 (m, 4H), 3.90 (s, 6H), 3.65 (s, 3H), 3.47-3.15 (m, 4H), 3.09- |
| | | 2.78 (m, 7H), 2.60 (d, J = 16.2 Hz, 2H), 2.46-2.34 (m, 2H), 2.17- |
| | | 2.08 (m, 1H), 1.98-1.87 (m, 6H), 1.86-1.82 (m, 3H), 1.49 (q, J = |
| | | 12.7 Hz, 2H). |
| D368 | 790.65 | ¹H NMR (400 MHz, DMSO-d6) δ 11.00 (s, 1H), 9.75 (s, 1H), 9.55 |
| | | (s, 1H), 9.32 (br s, 1H, TFA salt), 8.20 (s, 1H), 7.52 (dd, J = 8.4, 2.7 |
| | | Hz, 1H), 7.19-7.09 (m, 2H), 6.97 (s, 2H), 5.11 (dd, J = 13.3, 5.1 |
| | | Hz, 1H), 4.93-4.85 (m, 1H), 4.38 (d, J = 16.9 Hz, 2H), 4.26 (d, J = |
| | | 16.9 Hz, 2H), 3.92 (s, 6H), 3.68 (s, 3H), 3.54-3.38 (m, 4H), 3.25- |
| | | 3.21 (m, 1H), 3.06-2.82 (m, 6H), 2.67-2.56 (m, 2H), 2.44-2.38 |
| | | (m, 2H), 2.18-1.73 (m, 10H), 1.54-1.46 (m, 2H). |
| D369 | 749.25 | ¹H NMR (400 MHz, DMSO-d6) δ 10.95 (s, 1H), 10.05-9.61 (m, |
| | | 2H, TFA salt), 8.15 (s, 1H), 7.56-7.46 (m, 2H), 6.90 (d, J = 4.6 |
| | | Hz, 2H), 6.54-6.45 (m, 2H), 5.05 (dd, J = 13.2, 5.1 Hz, 1H), 4.40- |
| | | 4.28 (m, 2H), 4.27-4.15 (m, 4H), 4.12-3.98 (m, 2H), 3.90 (s, |
| | | 6H), 3.78 (s, 2H), 3.70 (s, 2H), 3.60 (d, J = 2.0 Hz, 3H), 3.52 (s, |
| | | 3H), 3.41 (s, 3H), 3.17 (s, 1H), 2.99-2.90 (m, 3H), 2.68-2.52 (m, |
| | | 2H), 2.47-2.28 (m, 1H), 2.13 (d, J = 13.9 Hz, 2H), 2.00-1.88 (m, 3H). |
| D370 | 749.4 | ¹H NMR (400 MHz, DMSO-d6) δ 10.95 (s, 1H), 9.99-9.58 (m, 2H, |
| | | TFA salt), 8.63 (s, 1H), 7.92 (s, 1H), 7.52 (d, J = 8.2 Hz, 1H), 7.41 |
| | | (s, 2H), 6.54-6.45 (m, 2H), 5.05 (dd, J = 13.2, 5.1 Hz, 1H), 4.42- |
| | | 4.27 (m, 2H), 4.25-4.14 (m, 4H), 4.08 (s, 3H), 4.05-3.95 (m, |
| | | 2H), 3.94 (s, 6H), 3.77 (s, 2H), 3.69 (s, 2H), 3.54 (s, 3H), 3.38 (s, |
| | | 3H), 3.17 (d, J = 6.7 Hz, 1H), 2.96 (s, 3H), 2.65-2.51 (m, 2H), 2.43- |
| | | 2.36 (m, 1H), 2.12 (d, J = 14.3 Hz, 2H), 2.00-1.88 (m, 3H). |
| D371 | 805.45 | 1H NMR (400 MHz, Methanol-d4) δ 7.86 (d, J = 9.7 Hz, 1H), 7.72 |
| | | (d, J = 8.4 Hz, 1H), 7.46 (s, 1H), 7.06-6.97 (m, 2H), 6.90-6.79 |
| | | (m, 3H), 5.14 (dd, J = 13.3, 5.1 Hz, 1H), 4.55-4.39 (m, 4H), 4.01- |
| | | 3.86 (m, 6H), 3.74 (s, 3H), 3.69-3.52 (m, 3H), 3.41-3.36 (m, |
| | | 1H), 3.28-3.16 (m, 2H), 3.13-2.98 (m, 4H), 2.96-2.86 (m, 2H), |
| | | 2.85-2.75 (m, 1H), 2.74-2.65 (m, 1H), 2.60-2.43 (m, 2H), 2.27 |
| | | (s, 1H), 2.22-2.15 (m, 1H), 2.14-1.92 (m, 8H), 1.73-1.59 (m, 2H). |

### Example 83—Preparation of Compounds 001-0010

In analogy to the procedures described in the examples above, compounds D01-D010 were prepared using the appropriate starting materials.

| Compound No. | LCMS | ¹H NMR |
|---|---|---|
| DD1 | 942.5 | ¹H NMR (300 MHz, DMSO-d6) δ 9.45 (s, 1H), 8.98 (s, 1H), 8.72 (d, |
| | | J = 5.7 Hz, 1H), 8.60 (t, J = 6.0 Hz, 1H), 8.19 (s, 1.0H, FA), 7.87 (s, |
| | | 1H), 7.56 (d, J = 5.6 Hz, 1H), 7.47-7.35 (m, 5H), 6.72 (s, 2H), 4.57 |
| | | (d, J = 9.5 Hz, 1H), 4.47-4.33 (m, 3H), 4.30-4.21 (m, 1H), 3.97 (s, |
| | | 2H), 3.80 (s, 6H), 3.68-3.50 (m, 18H), 2.58 (t, J = 6.1 Hz, 2H), 2.44 |
| | | (s, 3H), 2.18 (s, 3H), 2.11-2.00 (m, 1H), 1.97-1.85 (m, 1H), 0.95 |
| | | (s, 9H). |
| DD2 | 754.2 | ¹H NMR (400 MHz, DMSO-d6) δ 11.12 (s, 1H), 9.43 (s, 1H), 8.71 (d, |
| | | J = 5.8 Hz, 1H), 8.22 (s, 1, 5H, FA), 8.11 (s, 1H), 7.88-7.81 (m, 2H), |
| | | 7.54 (d, J = 5.6 Hz, 1H), 7.40 (s, 1H), 7.35 (d, J = 8.6 Hz, 1H), 6.72 |
| | | (s, 2H), 5.16-5.07 (m, 1H), 4.68 (s, 2H), 3.80 (s, 6H), 3.62-3.58 |
| | | (m, 5H), 3.31-3.10 (m, 7H), 2.93-2.83 (m, 1H), 2.46 (s, 2H), 2.21 |
| | | (s, 3H), 2.17 (s, 3H), 2.16-1.94 (m, 2H). |
| DD3 | 740.45 | ¹H NMR (300 MHz, Methanol-d4) δ 9.53 (s, 1H), 8.70 (d, J = 5.8 Hz, |
| | | 1H), 8.55 (s, 1H, FA), 7.74 (s, 1H), 7.62 (d, J = 5.7 Hz, 1H), 7.38 (t, J = |
| | | 8.1 Hz, 1H), 6.77 (s, 2H), 6.63 (d, J = 7.8 Hz, 1H), 6.44 (d, J = 8.4 |
| | | Hz, 1H), 5.21 (dd, J = 10.9, 5.7 Hz, 1H), 4.52-4.25 (m, 2H), 4.12- |
| | | 4.00 (m, 1H), 3.90 (s, 8H), 3.85-3.75 (m, 6H), 3.71 (s, 3H), 3.53- |
| | | 3.42 (m, 2H), 2.93-2.70 (m, 7H), 2.64 (s, 3H), 2.26-2.17 (m, 1H). |
| DD4 | 709.4 | ¹H NMR (300 MHz, DMSO-d6) δ 11.02 (s, 1H), 9.48 (s, 1H), 8.75 (d, |
| | | J = 5.7 Hz, 2H), 7.92 (s, 1H), 7.58 (d, J = 5.6 Hz, 1H), 7.29 (t, J = 7.7 |
| | | Hz, 1H), 6.93 (d, J = 7.4 Hz, 1H), 6.87 (s, 2H), 6.74 (d, J = 8.0 Hz, |
| | | 1H), 5.13 (dd, J = 13.2, 5.1 Hz, 1H), 4.37-4.26 (m, 2H), 4.22-4.13 |
| | | (m, 2H), 3.89 (s, 7H), 3.62 (s, 3H), 3.21-3.03 (m, 4H), 2.98-2.84 |
| | | (m, 1H), 2.77-2.63 (m, 3H), 2.30-2.23 (m, 1H), 2.10-1.98 (m, |
| | | 1H), 1.84-1.66 (m, 2H), 1.66-1.53 (m, 2H), 1.44-1.29 (m, 8H). |
| DD5 | 736.45 | ¹H NMR (400 MHz, Methanol-d4) δ 9.51 (s, 1H), 8.69 (d, J = 5.7 Hz, |
| | | 1H), 8.56 (s, 1H, FA), 7.76 (s, 1H), 7.61 (dd, J = 5.8, 0.9 Hz, 1H), |
| | | 7.47 (t, J = 8.1 Hz, 1H), 6.87 (s, 2H), 6.67 (d, J = 7.8 Hz, 1H), 6.46 |
| | | (d, J = 8.4 Hz, 1H), 5.19 (dd, J = 11.0, 5.7 Hz, 1H), 4.29 (s, 2H), 3.96 |
| | | (s, 6H), 3.68 (s, 3H), 3.37-3.36 (m, 1H), 3.14-3.02 (m, 3H), 2.91- |
| | | 2.70 (m, 6H), 2.63 (s, 3H), 2.24-2.17 (m, 1H), 1.87-1.76 (m, |
| | | 2H), 1.74-1.64 (m, 2H), 1.54-1.36 (m, 8H). |
| DD6 | 722.54 | ¹H NMR (400 MHz, DMSO-d6) δ 11.11 (s, 1H), 9.45 (s, 1H), 8.72 (d, |
| | | J = 5.7 Hz, 1H), 7.91 (d, J = 44.8 Hz, 2H), 7.55 (d, J = 5.7 Hz, 1H), |
| | | 6.84 (s, 2H), 5.22-5.02 (m, 0H), 4.98 (s, 1H), 4.71 (s, 1H), 4.35 (s, |
| | | 2H), 3.94-3.78 (m, 6H), 3.59 (s, 3H), 3.13-2.80 (m, 2H), 2.73 (s, |
| | | 2H), 2.67-2.53 (m, 1H), 2.05 (s, 2H). |
| DD7 | 800.3 | 1H NMR (400 MHz, DMSO-d6) δ 11.11 (s, 1H), 9.40 (s, 1H), 8.68 (d, |
| | | J = 5.6 Hz, 1H), 8.14-8.04 (m, 3H), 7.92 (d, J = 8.3 Hz, 1H), 7.83 |
| | | (s, 1H), 7.72 (d, J = 8.8 Hz, 2H), 7.53 (d, J = 5.6 Hz, 1H), 7.00 (d, J = |
| | | 8.7 Hz, 2H), 6.74 (s, 2H), 5.15 (dd, J = 12.9, 5.4 Hz, 1H), 4.06 (q, J = |
| | | 5.2 Hz, 1H), 4.02 (t, J = 6.4 Hz, 2H), 3.82 (s, 6H), 3.64 (s, 2H), |
| | | 3.56 (s, 3H), 3.29-3.12 (m, 5H), 3.00 (s, 3H), 2.95-2.82 (m, 1H), |
| | | 2.64-2.50 (m, 2H), 2.22 (s, 3H), 2.06 (d, J = 11.5 Hz, 1H), 1.71 (d, |
| | | J = 15.0 Hz, 0H), 1.71 (s, 2H), 1.59 (q, J = 7.3 Hz, 2H). |
| DD8 | 814.3 | 1H NMR (400 MHz, DMSO-d6) δ 11.11 (s, 1H), 9.40 (s, 1H), 8.68 (d, |
| | | J = 5.6 Hz, 1H), 8.23 (s, 1H), 8.10-8.01 (m, 2H), 7.90 (d, J = 8.3 |
| | | Hz, 1H), 7.83 (s, 1H), 7.76-7.64 (m, 3H), 7.52 (d, J = 6.2 Hz, 1H), |
| | | 7.00 (d, J = 8.8 Hz, 2H), 6.72 (s, 2H), 5.15 (dd, J = 12.9, 5.4 Hz, 1H), |
| | | 4.00 (t, J = 6.3 Hz, 2H), 3.81 (s, 6H), 3.58 (d, J = 14.7 Hz, 5H), 3.18 |
| | | (d, J = 6.3 Hz, 1H), 3.15 (s, 5H), 2.94 (s, 2H), 2.92-2.82 (m, 1H), |
| | | 2.62 (s, 1H), 2.59-2.50 (m, 1H), 2.16 (s, 3H), 2.07 (d, J = 11.7 Hz, |
| | | 1H), 1.73 (t, J = 7.0 Hz, 2H), 1.49 (d, J = 5.5 Hz, 2H), 1.42 (d, J = |
| | | 7.9 Hz, 3H). |
| DD9 | 571.61 | ¹H NMR (400 MHz, DMSO-d6) δ 8.32 (s, 1H), 8.09-8.02 (m, 1H), |
| | | 7.82-7.77 (m, 1H), 7.73 (s, 1H), 7.63 (s, 0H), 6.87 (d, J = 8.0 Hz, |
| | | 1H), 6.82 (s, 2H), 5.09 (dt, J = 11.9, 5.8 Hz, 1H), 3.92 (d, J = 3.9 Hz, |
| | | 5H), 3.86 (d, J = 3.7 Hz, 6H), 3.51 (d, J = 2.0 Hz, 4H), 3.15 (s, 1H), |
| | | 2.08 (d, J = 2.6 Hz, 4H). |
| DD10 | 803.2 | 1H NMR (300 MHz, DMSO) δ 11.13 (s, 1H), 8.20 (s, FA, 1H), 8.09 |
| | | (d, J = 8.3 Hz, 1H), 7.90 (d, J = 8.4 Hz, 1H), 7.83 (d, J = 8.1 Hz, 1H), |
| | | 7.69-7.59 (m, 1H), 7.58-7.49 (m, 1H), 7.43 (d, J = 7.5 Hz, 1H), |
| | | 7.34-7.23 (m, 2H), 6.73 (s, 2H), 5.12 (dd, J = 12.9, 5.4 Hz, 1H), |
| | | 5.05-4.94 (m, 1H), 3.81 (s, 6H), 3.73-3.67 (m, 1H), 3.03-2.89 (m, |
| | | 2H), 2.88-2.81 (m, 1H), 2.66-2.53 (m, 2H), 2.49-2.39 (m, 6H), |
| | | 2.36-2.21 (m, 6H), 2.14-1.99 (m, 3H), 1.89-1.75 (m, 2H), 1.72- |
| | | 1.45 (m, 7H), 1.26-1.06 (m, 2H). |

### Example 84—Preparation of Compounds 0372-0476

In analogy to the procedures described in the examples above, compounds D372-D476 were prepared using the appropriate starting materials.

| Compound No. | LCMS | ¹H NMR |
|---|---|---|
| D372 | 638.25 | ¹H NMR (400 MHz, DMSO-d6) δ 11.13 (s, 1H), 9.45 (s, 1H), 8.73 (d, |
| | | J = 5.7 Hz, 1H), 7.88 (d, J = 14.1 Hz, 2H), 7.57 (d, J = 5.6 Hz, 1H), |
| | | 7.36-7.28 (m, 2H), 6.79 (s, 2H), 5.18-5.01 (m, 2H), 4.25-3.92 (m, |
| | | 3H), 3.84 (s, 7H), 3.61 (s, 4H), 2.96-2.81 (m, 1H), 2.70-2.53 (m, |
| | | 3H), 2.10-2.01 (m, 1H). |
| D373 | 691.30 | ¹H NMR (400 MHz, DMSO-d6) δ 11.08 (s, 1H), 9.45 (d, J = 0.8 Hz, |
| | | 1H), 8.73 (d, J = 5.7 Hz, 1H), 8.18 (s, FA, 1H), 7.89 (s, 1H), 7.67- |
| | | 7.57 (m, 2H), 6.81-6.72 (m, 3H), 6.66 (dd, J = 8.4, 2.1 Hz, 1H), |
| | | 5.06 (dd, J = 12.9, 5.4 Hz, 1H), 3.82 (s, 6H), 3.74 (s, 4H), 3.58 (d, J = |
| | | 20.8 Hz, 6H), 2.95-2.82 (m, 1H), 2.63-2.52 (m, 2H), 2.46-2.41 |
| | | (m, 3H), 2.04-1.97 (m, 1H), 1.77-1.70 (m, 4H). |
| D374 | 677.30 | ¹H NMR (400 MHz, MeOD) δ 9.59 (s, 1H), 8.71 (d, J = 6.1 Hz, 1H), |
| | | 7.94 (s, 1H), 7.81 (d, J = 6.0 Hz, 1H), 7.67-7.60 (m, 1H), 6.91 (s, |
| | | 2H), 6.59 (d, J = 7.8 Hz, 2H), 5.11 (dd, J = 13.3, 5.1 Hz, 1H), 4.47 (s, |
| | | 2H), 4.40 (d, J = 7.0 Hz, 2H), 4.00 (s, 6H), 3.92 (s, 2H), 3.80 (s, 2H), |
| | | 3.75 (s, 3H), 3.62-3.55 (m, 3H), 3.31-3.21 (m, 1H), 2.98-2.85 (m, |
| | | 1H), 2.85-2.74 (m, 1H), 2.55-2.39 (m, 1H), 2.33-2.24 (m, 2H), |
| | | 2.21-2.06 (m, 3H). |
| D375 | 624.25 | ¹H NMR (300 MHz, Methanol-d4) δ 9.57 (s, 1H), 8.70 (d, J = 6.0 Hz, |
| | | 1H), 7.90 (s, 1H), 7.77 (dd, J = 14.9, 7.1 Hz, 2H), 7.09 (d, J = 10.5 |
| | | Hz, 2H), 6.89 (s, 2H), 5.41-5.20 (m, 1H), 5.15 (dd, J = 13.3, 5.2 |
| | | Hz, 1H), 4.86-4.60 (m, 4H), 4.49 (d, J = 4.5 Hz, 2H), 4.44-4.27 |
| | | (m, 2H), 3.97 (d, J = 14.5 Hz, 6H), 3.73 (s, 3H), 3.01-2.74 (m, 2H), |
| | | 2.60-2.41 (m, 1H), 2.25-2.13 (m, 1H). |
| D376 | 652.30 | ¹H NMR (400 MHz, DMSO-d6) δ 11.14 (s, 1H), 9.81 (s, TFA, 1H), |
| | | 9.48 (d, J = 0.8 Hz, 1H), 8.75 (d, J = 5.7 Hz, 1H), 7.96-7.89 (m, |
| | | 2H), 7.57 (d, J = 5.7 Hz, 1H), 7.44-7.34 (m, 2H), 6.88 (s, 2H), 5.15 |
| | | (dd, J = 12.8, 5.4 Hz, 2H), 4.74-4.57 (m, 2H), 4.55-4.42 (m, 2H), |
| | | 4.09 (s, 1H), 3.92 (s, 6H), 3.63 (s, 3H), 2.97-2.84 (m, 1H), 2.66- |
| | | 2.52 (m, 2H), 2.10-2.03 (m, 1H), 1.53 (d, J = 6.8 Hz, 3H). |
| D377 | 677.35 | ¹H NMR (400 MHz, DMSO-d6) δ 10.98 (s, 1H), 9.16 (s, 1H), 7.73 (s, |
| | | 1H), 7.41 (d, J = 8.5 Hz, 1H), 7.25 (dd, J = 8.5, 2.4 Hz, 1H), 7.14 (d, |
| | | J = 2.3 Hz, 1H), 6.74 (d, J = 20.0 Hz, 3H), 5.10 (dd, J = 13.3, 5.1 Hz, |
| | | 1H), 4.38-4.15 (m, 2H), 3.93 (s, 3H), 3.79 (s, 6H), 3.73 (d, J = 12.3 |
| | | Hz, 3H), 3.57-3.52 (m, 5H), 2.97-2.84 (m, 1H), 2.75-2.64 (m, |
| | | 2H), 2.64-2.55 (m, 1H), 2.48-2.38 (m, 4H), 2.38-2.20 (m, 6H), |
| | | 2.03-1.94 (m, 1H), 1.74 (d, J = 12.4 Hz, 2H), 1.52-1.42 (m, 1H), |
| | | 1.41-1.32 (m, 2H), 1.31-1.17 (m, 2H). |
| D378 | 624.30 | ¹H NMR (300 MHz, Methanol-d4) δ 9.56 (s, 1H), 8.69 (d, J = 5.9 Hz, |
| | | 1H), 7.84 (s, 1H), 7.68 (s, 1H), 7.60 (d, J = 8.3 Hz, 1H), 7.33-7.16 |
| | | (m, 2H), 6.89 (s, 2H), 5.40-5.07 (m, 2H), 4.84-4.61 (m, 4H), 4.59- |
| | | 4.44 (m, 2H), 4.44-4.28 (m, 2H), 4.07-3.85 (m, 6H), 3.73 (s, |
| | | 3H), 3.01-2.86 (m, 1H), 2.86-2.75 (m, 1H), 2.61-2.43 (m, 1H), |
| | | 2.25-2.14 (m, 1H). |
| D379 | 810.35 | ¹H NMR (400 MHz, Methanol-d4) δ 9.55 (s, 1H), 8.70 (d, J = 5.8 Hz, |
| | | 1H), 8.56 (s, 1H, FA), 7.78 (s, 1H), 7.69-7.56 (m, 2H), 6.88 (s, 2H), |
| | | 6.65-6.53 (m, 2H), 5.11 (dd, J = 13.3, 5.2 Hz, 1H), 4.53-4.24 (m, |
| | | 4H), 4.06 (d, 2H), 3.98 (s, 6H), 3.76 (d, J = 8.0 Hz, 2H), 3.72 (s, 3H), |
| | | 3.56-3.48 (m, 2H), 3.16-3.01 (m, 2H), 2.99-2.85 (m, 1H), 2.84- |
| | | 2.64 (m, 3H), 2.60-2.40 (m, 3H), 2.36 (s, 2H), 2.21-2.11 (m, |
| | | 3H), 2.05 (d, J = 13.9 Hz, 2H), 1.92 (s, 1H), 1.59-1.38 (m, 2H). |
| D380 | 661.35 | 1H), 9.49 (s, 1H), 8.75 (d, J = 5.9 Hz, 1H), 7.99 (s, 1H), 7.56 (d, J = |
| | | 5.9 Hz, 1H), 7.39 (d, J = 8.5 Hz, 1H), 7.12 (t, J = 2.0 Hz, 1H), 7.04 |
| | | (d, J = 2.0 Hz, 2H), 6.74-6.67 (m, 2H), 5.08 (dd, J = 13.3, 5.1 Hz, |
| | | 1H), 4.32 (d, J = 16.6 Hz, 1H), 4.19 (d, J = 16.6 Hz, 1H), 3.86 (s, |
| | | 3H), 3.67 (s, 6H), 3.61 (s, 3H), 3.39 (s, 2H), 2.98-2.84 (m, 1H), 2.63- |
| | | 2.59 (m, 1H), 2.42-2.33 (m, 1H), 2.02-1.95 (m, 1H), 1.90-1.72 |
| | | (m, 4H). |
| D381 | 767.40 | ¹H NMR (400 MHz, DMSO-d6) δ 10.86-10.81 (m, HCl, 1H), 9.52 |
| | | (s, 1H), 8.80-8.73 (m, 1H), 8.52 (s, 3H), 8.07 (s, 1H), 7.93 (d, J = |
| | | 8.2 Hz, 1H), 7.72 (s, 1H), 7.44-7.34 (m, 2H), 6.88 (d, J = 6.1 Hz, |
| | | 2H), 5.93-5.84 (m, 1H), 5.75-5.67 (m, 1H), 5.53-5.21 (m, 2H), |
| | | 4.81-4.73 (m, 1H), 4.67-4.53 (m, 1H), 4.52-4.44 (m, 2H), 4.33- |
| | | 4.29 (m, 1H), 4.17-4.12 (m, 1H), 3.92 (s, 3H), 3.87 (s, 4H), 3.64 (s, |
| | | 3H), 3.13-3.05 (m, 1H), 2.93-2.84 (m, 1H), 2.70-2.56 (m, 1H), |
| | | 2.18-2.11 (m, 2H), 0.98-0.90 (m, 6H). |
| D382 | 663.35 | ¹H NMR (300 MHz, DMSO-d6) δ 11.51 (s, 1H), 9.47 (s, 1H), 8.92 (s, |
| | | 1H, FA), 8.76 (d, J = 5.7 Hz, 1H), 7.90 (s, 1H), 7.59 (d, J = 5.7 Hz, |
| | | 1H), 7.40 (d, J = 8.2 Hz, 1H), 6.87 (s, 2H), 6.75-6.52 (m, 2H), 5.19 |
| | | (dd, J = 9.1, 6.3 Hz, 1H), 4.49 (d, J = 16.8 Hz, 1H), 4.38-4.15 (m, |
| | | 3H), 3.91 (s, 7H), 3.76 (s, 2H), 3.62 (s, 6H), 3.21-3.04 (m, 2H), |
| | | 3.04-2.79 (m, 2H), 2.21-1.90 (m, 4H). |
| D383 | 667.30 | ¹H NMR (400 MHz, DMSO-d6 with a drop of D₂O) δ 9.46 (s, 1H), |
| | | 8.75 (d, J = 5.7 Hz, 1H), 7.88 (s, 1H), 7.67 (d, J = 8.2 Hz, 1H), 7.60 |
| | | (d, J = 5.6 Hz, 1H), 6.86 (s, 2H), 6.78 (d, J = 2.0 Hz, 1H), 6.67 (dd, J = |
| | | 8.4, 2.1 Hz, 1H), 5.21 (dd, J = 9.6, 5.6 Hz, 1H), 4.29 (s, 2H), 3.91 |
| | | (d, J = 8.4 Hz, 8H), 3.81 (s, 2H), 3.61 (s, 3H), 3.38 (d, J = 12.6 Hz, |
| | | 2H), 3.13 (t, J = 12.0 Hz, 2H), 3.05-2.95 (m, 1H), 2.82 (dd, J = |
| | | 17.9, 5.5 Hz, 1H), 2.15 (d, J = 14.1 Hz, 2H), 2.01 (t, J = 12.8 Hz, 2H). |
| D384 | 753.40 | ¹H NMR (400 MHz, DMSO-d6) δ 10.71 (s, 1H, HCl salt), 9.49 (s, |
| | | 1H), 8.79-8.72 (m, 1H), 8.51 (br s, 3H), 7.99 (s, 1H), 7.71 (d, J = |
| | | 8.4 Hz, 1H), 7.67-7.59 (m, 1H), 7.39-7.01 (m, 3H), 6.87 (d, J = |
| | | 5.1 Hz, 2H), 5.86-5.78 (m, 1H), 5.76-5.68 (m, 1H), 5.35-5.02 |
| | | (m, 2H), 4.80-4.67 (m, 1H), 4.60-4.54 (m, 1H), 4.52-4.41 (m, |
| | | 3H), 4.34-4.22 (m, 2H), 4.16-4.10 (m, 1H), 3.94-3.82 (m, 7H), |
| | | 3.63 (s, 3H), 3.20-3.06 (m, 1H), 2.91-2.82 (m, 1H), 2.46-2.36 |
| | | (m, 1H), 2.21-2.06 (m, 2H), 0.97-0.90 (m, 6H). |
| D385 | 636.35 | ¹H NMR (300 MHz, DMSO-d6) δ 11.12 (s, 1H), 9.45 (s, 1H), 8.73 (d, |
| | | J = 5.7 Hz, 1H), 8.13 (s, 0.1H, FA salt), 7.90-7.81 (m, 2H), 7.51 (d, |
| | | J = 5.7 Hz, 1H), 7.37-7.27 (m, 2H), 6.94 (s, 2H), 5.18-4.99 (m, |
| | | 2H), 3.84 (s, 6H), 3.60 (s, 4H), 3.28-3.25 (m, 2H), 2.99-2.72 (m, |
| | | 3H), 2.61-2.53 (m, 2H), 2.11-1.99 (m, 1H), 1.21 (t, J = 7.5 Hz, 3H). |
| D386 | 650.30 | ¹H NMR (300 MHz, DMSO-d6) δ 11.12 (s, 1H), 9.45 (s, 1H), 8.73 |
| | | (dd, J = 5.7, 2.3 Hz, 1H), 8.13 (s, 0.1H, FA salt), 7.89-7.80 (m, 2H), |
| | | 7.49 (dd, J = 6.0, 2.1 Hz, 1H), 7.37-7.28 (m, 2H), 6.92 (s, 2H), 5.12 |
| | | (dd, J = 12.9, 5.3 Hz, 1H), 4.63 (s, 1H), 3.90-3.68 (m, 6H), 3.60 (s, |
| | | 4H), 3.44-3.37 (m, 1H), 2.99-2.72 (m, 3H), 2.62-2.52 (m, 2H), |
| | | 2.11-1.95 (m, 1H), 1.29-1.12 (m, 6H). |
| D387 | 647.25 | ¹H NMR (300 MHz, DMSO-d6) δ 10.97 (s, 1H), 9.45 (s, 1H), 8.73 (d, |
| | | J = 5.7 Hz, 1H), 7.85 (s, 1H), 7.48-7.36 (m, 2H), 7.01-6.89 (m, |
| | | 3H), 6.73-6.65 (m, 2H), 5.11-5.04 (m, 1H), 4.31-4.18 (m, 2H), |
| | | 3.82 (s, 3H), 3.60 (s, 7H), 3.52 (s, 2H), 2.94-2.87 (m, 1H), 2.67- |
| | | 2.61 (m, 1H), 2.46-2.34 (m, 4H), 2.04-1.92 (m, 2H), 1.83-1.73 |
| | | (m, 4H). |
| D388 | 705.45 | ¹H NMR (300 MHz, DMSO-d6) δ 10.78 (s, 1H), 9.48 (s, 1H), 9.07 (br s, |
| | | 1H), 8.76 (d, J = 5.7 Hz, 1H), 7.92 (s, 1H), 7.68 (d, J = 8.2 Hz, |
| | | 1H), 7.61 (d, J = 5.8 Hz, 1H), 6.88 (s, 2H), 6.78 (d, J = 2.1 Hz, 1H), |
| | | 6.67 (dd, J = 8.3, 2.2 Hz, 1H), 5.14 (dd, 1H), 4.38-4.24 (m, 2H), |
| | | 3.97-3.88 (m, 8H), 3.83 (s, 2H), 3.63 (s, 3H), 3.46-3.35 (m, 3H), |
| | | 3.18-3.06 (m, 3H), 2.76-2.66 (m, 1H), 2.22-2.13 (m, 2H), 2.11- |
| | | 1.80 (m, 5H). |
| D389 | 663.30 | ¹H NMR (300 MHz, DMSO-d6) δ 9.45 (s, 1H), 8.74 (d, J = 5.7 Hz, |
| | | 1H), 7.89 (s, 1H), 7.73 (s, 1H), 7.58 (d, J = 5.6 Hz, 1H), 7.36 (d, J = |
| | | 8.7 Hz, 1H), 6.80 (s, 2H), 6.70-6.64 (m, 2H), 4.64 (dd, J = 10.8, 6.8 |
| | | Hz, 1H), 4.38-4.25 (m, 1H), 4.21-4.07 (m, 1H), 3.96-3.80 (m, |
| | | 8H), 3.66-3.59 (m, 7H), 3.23-3.11 (m, 3H), 2.94-2.74 (m, 3H), |
| | | 2.09-1.80 (m, 8H). |
| D390 | 663.50 | ¹H NMR (300 MHz, DMSO-d6) δ 9.48 (s, 1H), 8.99 (s, 1H, TFA), |
| | | 8.76 (d, J = 5.7 Hz, 1H), 7.91 (s, 1H), 7.60 (d, J = 5.7 Hz, 1H), 7.54 |
| | | (d, J = 2.5 Hz, 1H), 7.40 (d, J = 8.5 Hz, 1H), 6.88 (s, 2H), 6.73-6.64 |
| | | (m, 2H), 4.41-4.27 (m, 5H), 3.92 (s, 6H), 3.76 (s, 3H), 3.64 (s, 4H), |
| | | 3.45-3.34 (m, 2H), 3.34-3.06 (m, 4H), 2.44-2.29 (m, 2H), 2.19- |
| | | 1.85 (m, 6H). |
| D391 | 667.45 | ¹H NMR (300 MHz, DMSO-d6) δ 10.95 (s, 1H), 9.54 (s, 1H), 9.10 (s, |
| | | 1H), 8.97 (d, J = 8.2 Hz, 1H), 8.82 (d, J = 5.7 Hz, 1H), 8.37 (d, J = |
| | | 5.9 Hz, 1H), 7.96 (s, 1H), 7.65 (d, J = 5.7 Hz, 1H), 6.94 (s, 2H), 6.60 |
| | | (d, J = 6.0 Hz, 1H), 4.85-4.71 (m, 1H), 4.42-4.32 (m, 2H), 4.12-3.89 |
| | | (m, 11H), 3.69 (s, 3H), 3.27-3.15 (m, 3H), 2.98-2.78 (m, 1H), 2.71- |
| | | 2.60 (m, 1H), 2.32-1.99 (m, 6H). |
| D392 | 665.25 | ¹H NMR (300 MHz, Methanol-d4) δ 9.63 (s, 1H), 8.72 (d, J = 6.4 Hz, |
| | | 1H), 8.08 (s, 1H), 7.95 (d, J = 6.3 Hz, 1H), 7.32 (t, J = 7.8 Hz, 1H), |
| | | 7.23 (dt, J = 7.9, 1.2 Hz, 1H), 7.02 (t, J = 2.0 Hz, 1H), 6.91 (s, 2H), |
| | | 6.71 (ddd, J = 8.0, 2.5, 1.1 Hz, 1H), 4.86-4.85 (m, 1H), 4.46 (s, |
| | | 2H), 4.00 (s, 6H), 3.85 (s, 2H), 3.75 (d, J = 9.0 Hz, 5H), 3.58 (d, J = |
| | | 12.9 Hz, 2H), 3.25 (t, J = 11.8 Hz, 2H), 2.95-2.65 (m, 2H), 2.34- |
| | | 2.05 (m, 6H). |
| D393 | 571.25 | ¹H NMR (300 MHz, DMSO-d6) δ 11.00 (s, 1H), 9.45 (d, J = 0.8 Hz, |
| | | 1H), 8.73 (d, J = 5.7 Hz, 1H), 8.19 (.1.0 FA, s, 1H), 7.89 (s, 1H), 7.63- |
| | | 7.50 (m, 2H), 7.36 (s, 1H), 6.74 (s, 2H), 5.27 (dd, J = 11.5, 5.1 Hz, |
| | | 1H), 3.83 (s, 6H), 3.60 (d, J = 4.2 Hz, 5H), 2.92 (d, J = 11.3 Hz, 2H), |
| | | 2.84-2.68 (m, 1H), 2.68-2.53 (m, 1H), 2.49-2.35 (m, 2H), 2.24- |
| | | 2.09 (m, 3H), 1.80 (d, J = 12.8 Hz, 2H), 1.58-1.38 (m, 2H). |
| D394 | 613.25 | ¹H NMR (300 MHz, Methanol-d4) δ 9.60 (s, 1H), 8.71 (d, 1H), 7.97 |
| | | (s, 1H), 7.85 (d, 1H), 6.91 (s, 2H), 6.70 (s, 1H), 6.12 (dd, 1H), 4.48 |
| | | (s, 2H), 4.00 (s, 6H), 3.75 (s, 4H), 3.71 (s, 1H), 3.58-3.42 (m, 1H), |
| | | 3.27 (s, 1H), 3.06-2.95 (m, 1H), 2.90-2.76 (m, 2H), 2.60 (d, 3H), |
| | | 2.40-2.27 (m, 2H), 2.12 (dd, 4H). |
| D395 | 667.45 | ¹H NMR (300 MHz, DMSO-d6) δ 10.87 (s, 1H), 9.45 (s, 1H), 9.00 (d, |
| | | J = 8.4 Hz, 1H), 8.73 (d, J = 5.6 Hz, 1H), 8.54 (d, J = 1.2 Hz, 1H), |
| | | 8.17 (s, 1H, FA), 7.88 (s, 1H), 7.63-7.55 (m, 1H), 6.89 (d, J = 1.2 |
| | | Hz, 1H), 6.74 (s, 2H), 4.76 (ddd, J = 12.9, 8.4, 5.3 Hz, 1H), 3.82 (d, J = |
| | | 4.1 Hz, 10H), 3.61 (s, 3H), 3.58 (s, 2H), 2.84-2.74 (m, 1H), 2.55 |
| | | (s, 2H), 2.48-2.40 (m, 3H), 2.25-2.13 (m, 1H), 2.05-1.93 (m, |
| | | 1H), 1.75 (s, 4H). |
| D396 | 640.31 | ¹H NMR (400 MHz, DMSO-d6) δ 10.97 (s, 1H), 8.19 (s, FA, 1H), |
| | | 7.41-7.26 (m, 2H), 6.72-6.66 (m, 2H), 6.53 (s, 2H), 5.09 (dd, J = |
| | | 13.2, 5.0 Hz, 1H), 4.31 (d, J = 16.6 Hz, 1H), 4.18 (d, J = 16.6 Hz, |
| | | 1H), 3.79 (s, 6H), 3.77 (d, J = 7.1 Hz, 2H), 3.74-3.66 (m, 4H), 3.54 |
| | | (s, 3H), 2.97-2.84 (m, 1H), 2.77 (s, 2H), 2.64-2.59 (m, 2H), 2.40- |
| | | 2.28 (m, 5H), 2.04 (s, 3H), 2.01-1.95 (m, 3H). |
| D397 | 751.4 | ¹H NMR (400 MHz, DMSO-d6) δ 10.98 (s, 1H), 9.19 (br s, TFA salt, |
| | | 2H), 7.45-7.38 (m, 1H), 7.28 (s, 1H), 6.74-6.66 (m, 4H), 5.07 (dd, |
| | | J = 13.3, 5.1 Hz, 1H), 4.37-4.16 (m, 4H), 3.87 (s, 6H), 3.74 (d, J = |
| | | 8.5 Hz, 2H), 3.67 (d, J = 6.9 Hz, 2H), 3.65 (s, 5H), 3.53-3.50 (m, |
| | | 2H), 3.21 (s, 1H), 3.07-2.85 (m, 6H), 2.65-2.55 (m, 1H), 2.43- |
| | | 2.31 (m, 4H), 2.20-2.07 (m, 3H), 2.05 (s, 3H), 2.02-1.87 (m, 5H), |
| | | 1.58-1.39 (m, 2H). |
| D398 | 761.2 | 1H), 8.40 (d, J = 2.6 Hz, 1H), 8.17 (s, 1H, FA), 8.15 (d, J = 2.5 Hz, |
| | | 1H), 7.65 (d, J = 8.5 Hz, 1H), 7.30 (d, J = 2.2 Hz, 1H), 7.22 (dd, J = |
| | | 8.7, 2.3 Hz, 1H), 7.14-6.71 (m, 3H), 5.06 (dd, J = 12.8, 5.3 Hz, |
| | | 1H), 4.03 (d, J = 13.0 Hz, 2H), 3.85 (s, 6H), 3.59 (s, 3H), 3.54 (s, |
| | | 2H), 3.00-2.80 (m, 3H), 2.65-2.52 (m, 2H), 2.48-2.24 (m, 10H), |
| | | 2.06-1.96 (m, 1H), 1.73 (d, J = 12.6 Hz, 2H), 1.60-1.54 (m, 1H), |
| | | 1.37 (t, J = 7.3 Hz, 2H), 1.16 (q, J = 11.6 Hz, 2H). |
| D399 | 747.3 | ¹H NMR (300 MHz, DMSO-d6) δ 10.99 (s, 1H), 8.40 (d, J = 2.6 Hz, |
| | | 1H), 8.19 (s, 1H, FA), 8.15 (d, J = 2.6 Hz, 1H), 7.40 (d, J = 8.5 Hz, |
| | | 1H), 7.24 (dd, J = 8.6, 2.3 Hz, 1H), 7.14 (d, J = 2.3 Hz, 1H), 7.12- |
| | | 6.72 (m, 3H), 5.10 (dd, J = 13.2, 5.1 Hz, 1H), 4.33 (d, J = 16.7 Hz, |
| | | 1H), 4.19 (d, J = 16.7 Hz, 1H), 3.85 (s, 6H), 3.72 (d, J = 12.1 Hz, |
| | | 2H), 3.59 (s, 3H), 3.53 (s, 2H), 3.01-2.82 (m, 1H), 2.78-2.51 (m, |
| | | 4H), 2.46-2.20 (m, 10H), 1.98 (d, J = 13.3 Hz, 1H), 1.73 (d, J = |
| | | 12.4 Hz, 2H), 1.47-1.36 (m, 3H), 1.24 (q, J = 11.2 Hz, 2H). |
| D400 | 751.5 | ¹H NMR (400 MHz, DMSO-d6) δ 10.98 (s, 1H), 8.63 (s, 1H), 7.91 (s, |
| | | 1H), 7.47-7.39 (m, 3H), 7.29 (dd, J = 8.5, 2.4 Hz, 1H), 7.20 (d, J = |
| | | 2.3 Hz, 1H), 5.09 (dd, J = 13.3, 5.1 Hz, 1H), 4.40-4.14 (m, 4H), |
| | | 4.08 (s, 3H), 3.92 (s, 6H), 3.78-3.74 (m, 8H), 3.54 (s, 3H), 3.14 (s, |
| | | 4H), 2.91 (ddd, J = 17.2, 13.6, 5.4 Hz, 1H), 2.75 (t, J = 11.8 Hz, 2H), |
| | | 2.65-2.56 (m, 1H), 2.43-2.34 (m, 1H), 2.04-1.95 (m, 1H), 1.78 |
| | | (d, J = 12.4 Hz, 2H), 1.64-1.46 (m, 3H), 1.38-1.23 (m, 2H). |
| D401 | 690.3 | ¹H NMR (300 MHz, DMSO-d6) δ 10.98 (s, 1H), 8.37 (s, 1H), 8.24 (s, |
| | | 1H), 8.15 (s, 1H, FA), 7.41-7.32 (m, 1H), 7.20-6.73 (m, 3H), 6.68 |
| | | (dd, J = 5.1, 2.5 Hz, 2H), 5.09 (dd, J = 13.3, 5.0 Hz, 1H), 4.45-4.10 |
| | | (m, 2H), 4.00 (t, J = 7.5 Hz, 2H), 3.87 (s, 6H), 3.81-3.66 (m, 6H), |
| | | 2.94-2.83 (m, 1H), 2.75 (s, 2H), 2.64-2.52 (m, 2H), 2.42-2.25 |
| | | (m, 2H), 1.98 (t, J = 7.0 Hz, 3H), 1.75 (q, J = 7.5 Hz, 2H), 0.91 (t, J = |
| | | 7.3 Hz, 3H). |
| D402 | 749.35 | ¹H NMR (400 MHz, DMSO-d6) δ 10.98 (s, 1H), 9.95-9.63 (m, 2H, |
| | | TFA salt), 8.63 (s, 1H), 7.92 (s, 1H), 7.45-7.38 (m, 3H), 6.73-6.66 |
| | | (m, 2H), 5.07 (dd, J = 13.3, 5.1 Hz, 1H), 4.43-4.28 (m, 3H), 4.24- |
| | | 4.15 (m, 3H), 4.08 (s, 3H), 4.03-3.98 (m, 2H), 3.94 (s, 6H), 3.76- |
| | | 3.62 (m, 4H), 3.54 (s, 3H), 3.22-3.12 (m, 2H), 2.97-2.88 (m, 4H), |
| | | 2.70-2.56 (m, 2H), 2.44-2.30 (m, 2H), 2.12 (d, J = 14.1 Hz, 2H), |
| | | 2.02-1.89 (m, 3H). |
| D403 | 662.15 | ¹H NMR (400 MHz, DMSO-d6) δ 10.97 (s, 1H), 8.41 (s, J = 2.6 Hz, |
| | | 1H), 8.16 (s, 1H, FA), 7.37 (d, J = 8.8 Hz, 1H), 6.86-7.08 (m, 3H), |
| | | 6.72-6.65 (m, 2H), 5.08 (dd, J = 13.3, 5.1 Hz, 1H), 4.31 (d, J = |
| | | 16.6 Hz, 1H), 4.18 (d, J = 16.6 Hz, 1H), 3.87 (s, 6H), 3.79-3.67 (m, |
| | | 6H), 3.60 (s, 3H), 2.97-2.84 (m, 2H), 2.76 (s, 2H), 2.63-2.55 (m, |
| | | 3H), 2.31-2.42 (m, 2H), 2.02-1.94 (m, 3H). |
| D404 | 688.15 | ¹H NMR (300 MHz, DMSO-d6) δ 10.98 (s, 1H), 8.34 (d, J = 2.6 Hz, |
| | | 1H), 8.22 (s, 1H), 8.18 (s, 1H, FA), 7.37 (d, J = 8.8 Hz, 1H), 7.16- |
| | | 6.73 (m, 3H), 6.68 (dq, J = 4.0, 2.3 Hz, 2H), 6.03 (ddd, J = 17.2, |
| | | 10.5, 5.3 Hz, 1H), 5.33-5.00 (m, 3H), 4.68 (d, J = 5.5 Hz, 2H), 4.31 |
| | | (d, J = 16.7 Hz, 1H), 4.18 (d, J = 16.6 Hz, 1H), 3.90-3.79 (m, 7H), |
| | | 3.77-3.71 (m, 2H), 3.72-3.63 (m, 3H), 3.00-2.82 (m, 1H), 2.76 |
| | | (s, 2H), 2.59 (d, J = 17.1 Hz, 3H), 2.43-2.28 (m, 1H), 1.98 (t, J = |
| | | 6.9 Hz, 3H). |
| D405 | 777.4 | ¹H NMR (400 MHz, DMSO-d6) δ 10.98 (s, 1H), 8.61 (s, 1H), 8.19- |
| | | 8.14 (m, 1H, FA), 7.89-7.82 (m, 1H), 7.37 (d, J = 8.0 Hz, 1H), 7.36- |
| | | 7.29 (m, 2H), 6.72-6.65 (m, 2H), 5.08 (dd, J = 13.3, 5.1 Hz, 1H), |
| | | 4.31 (d, J = 16.6 Hz, 1H), 4.18 (d, J = 16.6 Hz, 1H), 4.08 (s, 3H), |
| | | 3.87 (s, 6H), 3.79 (s, 2H), 3.58 (s, 4H), 3.53 (s, 3H), 3.08-3.01 (m, |
| | | 2H), 2.98-2.84 (m, 1H), 2.68-2.55 (m, 2H), 2.43-2.30 (m, 6H), |
| | | 2.11 (d, J = 7.0 Hz, 2H), 2.02-1.93 (m, 1H), 1.77-1.67 (m, 6H), |
| | | 1.59 (s, 1H), 1.21-1.17 (m, 2H). |
| D406 | 676.3 | ¹H NMR (300 MHz, DMSO-d6) δ 11.08 (s, 1H), 8.41 (d, J = 2.6 Hz, |
| | | 1H), 8.19 (s, 1H, FA), 8.15 (d, J = 2.5 Hz, 1H), 7.63 (d, J = 8.3 Hz, |
| | | 1H), 7.15-6.74 (m, 4H), 6.64 (dd, J = 8.3, 2.1 Hz, 1H), 5.06 (dd, J = |
| | | 12.8, 5.4 Hz, 1H), 3.99-3.85 (m, 4H), 3.87 (s, 6H), 3.70 (s, 2H), |
| | | 3.59 (s, 3H), 2.97-2.80 (m, 1H), 2.77 (s, 2H), 2.63-2.53 (m, 4H), |
| | | 2.02 (t, J = 7.0 Hz, 3H). |
| D407 | 773.89 | ¹H NMR (400 MHz, DMSO-d6) δ 10.98 (s, 1H), 9.42 (s, 1H, TFA), |
| | | 9.25 (s, 1H, TFA), 8.50 (d, J = 2.7 Hz, 1H), 8.21 (d, J = 2.5 Hz, 1H), |
| | | 7.44-7.38 (m, 1H), 7.11-6.79 (m, 3H), 6.70 (dd, J = 5.8, 2.4 Hz, |
| | | 2H), 5.07 (dd, J = 13.3, 5.1 Hz, 1H), 4.38-4.14 (m, 4H), 3.95 (s, |
| | | 6H), 3.66 (d, J = 7.8 Hz, 2H), 3.61 (s, 3H), 3.54-3.39 (m, 4H), 3.21- |
| | | 3.14 (m, 1H), 3.02-2.82 (m, 7H), 2.64-2.56 (m, 1H), 2.43-2.35 |
| | | (m, 1H), 2.15-2.07 (m, 3H), 2.02-1.88 (m, 5H), 1.48 (q, J = 12.8 |
| | | Hz, 2H), 1.26 (q, J = 7.2, 6.7 Hz, 1H). |
| D408 | 662.3 | ¹H NMR (300 MHz, DMSO-d6) δ 10.95 (s, 1H), 8.41 (d, J = 2.6 Hz, |
| | | 1H), 8.21 (s, 1H, FA), 8.17-8.10 (m, 1H), 7.48 (d, J = 8.2 Hz, 1H), |
| | | 7.18-6.73 (m, 3H), 6.54-6.42 (m, 2H), 5.03 (dd, J = 13.3, 5.1 Hz, |
| | | 1H), 4.30 (d, J = 16.9 Hz, 1H), 4.16 (d, J = 16.9 Hz, 1H), 3.87 (s, |
| | | 6H), 3.79 (q, J = 7.9 Hz, 4H), 3.70 (s, 2H), 3.59 (s, 3H), 2.99-2.81 |
| | | (m, 1H), 2.80-2.74 (m, 2H), 2.63-2.52 (m, 2H), 2.41-2.29 (m, |
| | | 2H), 2.05-1.89 (m, 3H). |
| D409 | 676.25 | ¹H NMR (300 MHz, Methanol-d4) δ 8.55 (s, 1H, FA), 7.68 (s, 1H), |
| | | 7.42 (d, J = 8.2 Hz, 1H), 7.03-6.77 (m, 3H), 6.71 (s, 2H), 5.14 (dd, |
| | | J = 13.3, 5.1 Hz, 1H), 4.48-4.33 (m, 4H), 4.03-3.89 (m, 10H), |
| | | 3.71 (s, 3H), 3.64-3.54 (m, 2H), 3.47-3.37 (m, 2H), 3.00-2.85 |
| | | (m, 1H), 2.85-2.74 (m, 1H), 2.58-2.41 (m, 6H), 2.23-2.13 (m, 1H). |
| D410 | 787.25 | ¹H NMR (300 MHz, DMSO-d6) δ 10.98 (s, 1H), 8.17 (s, 1H, FA), |
| | | 7.61 (s, 1H), 7.37 (d, J = 8.1 Hz, 1H), 6.91 (t, J = 55.2 Hz, 1H), 6.74- |
| | | 6.65 (m, 2H), 6.61 (s, 2H), 5.08 (dd, J = 13.3, 5.1 Hz, 1H), 4.32 (d, |
| | | J = 16.6 Hz, 1H), 4.18 (d, J = 16.6 Hz, 1H), 3.81 (s, 6H), 3.69 (s, |
| | | 2H), 3.63-3.58 (m, 6H), 3.01-2.88 (m, 4H), 2.65-2.59 (m, 1H), |
| | | 2.41 (s, 4H), 2.37-2.26 (m, 6H), 2.11 (d, J = 6.9 Hz, 2H), 2.02- |
| | | 1.95 (m, 1H), 1.79-1.64 (m, 6H), 1.54 (s, 1H), 1.21-1.08 (m, 2H). |
| D411 | 680.35 | ¹H NMR (400 MHz, DMSO-d6) δ 10.96 (s, 1H), 9.06 (s, 1H, TFA |
| | | salt), 8.15 (s, 1H), 7.56-7.48 (m, 2H), 6.91 (s, 2H), 6.55-6.46 (m, |
| | | 2H), 5.05 (dd, J = 13.3, 5.1 Hz, 1H), 4.37-4.25 (m, 3H), 4.19 (d, J = |
| | | 16.9 Hz, 1H), 3.92 (s, 6H), 3.81 (s, 2H), 3.70 (s, 2H), 3.63 (s, 3H), |
| | | 3.52 (s, 3H), 3.13 (q, J = 11.1 Hz, 2H), 2.91 (ddd, J = 17.1, 13.6, 5.3 |
| | | Hz, 1H), 2.64-2.55 (m, 3H), 2.44-2.28 (m, 1H), 2.14 (d, J = 13.9 |
| | | Hz, 2H), 2.05-1.92 (m, 3H).19F NMR (377 MHz, DMSO-d6) δ −73.65. |
| D412 | 680.35 | ¹H NMR (400 MHz, DMSO-d6) δ 10.95 (s, 1H), 8.90 (s, 1H, TFA |
| | | salt), 8.63 (s, 1H), 7.93 (s, 1H), 7.52 (d, J = 8.3 Hz, 1H), 7.43 (s, |
| | | 2H), 6.55-6.46 (m, 2H), 5.05 (dd, J = 13.3, 5.1 Hz, 1H), 4.36-4.15 |
| | | (m, 4H), 4.09 (s, 3H), 3.95 (s, 6H), 3.80 (s, 2H), 3.69 (s, 2H), 3.55 (s, |
| | | 3H), 3.14-3.07 (m, 2H), 2.97-2.84 (m, 1H), 2.71-2.57 (m, 3H), |
| | | 2.39-2.31 (m, 1H), 2.17-2.09 (m, 2H), 2.04-1.93 (m, 3H). 19F |
| | | NMR (377 MHz, DMSO-d6) δ −73.66. |
| D413 | 694.45 | ¹H NMR (400 MHz, DMSO-d6) δ 11.08 (s, 1H), 8.19-8.14 (m, 1H, |
| | | FA), 8.12 (s, 1H), 7.64 (d, J = 8.3 Hz, 1H), 7.52 (s, 1H), 6.81-6.74 |
| | | (m, 3H), 6.69-6.62 (m, 1H), 5.06 (dd, J = 12.9, 5.4 Hz, 1H), 3.82 (s, |
| | | 6H), 3.74 (s, 4H), 3.61-3.54 (m, 5H), 3.51 (s, 3H), 2.95-2.82 (m, |
| | | 1H), 2.63-2.52 (m, 2H), 2.46-2.39 (m, 4H), 2.06-1.97 (m, 1H), |
| | | 1.78-1.69 (m, 4H). |
| D414 | 680.4 | ¹H NMR (400 MHz, DMSO-d6) δ 10.96 (s, 1H), 8.16 (s, 1H, FA), |
| | | 8.12 (s, 1H), 7.51 (s, 1H), 7.37 (d, J = 8.0 Hz, 1H), 6.76 (s, 2H), 6.72- |
| | | 6.65 (m, 2H), 5.08 (dd, J = 13.3, 5.2 Hz, 1H), 4.36-4.12 (m, 2H), |
| | | 3.82 (s, 6H), 3.61-3.55 (m, 9H), 3.51 (s, 3H), 2.94-2.85 (m, 1H), |
| | | 2.66-2.54 (m, 2H), 2.46-2.40 (m, 4H), 2.02-1.95 (m, 1H), 1.76- |
| | | 1.69 (m, 4H). |
| D415 | 694.3 | ¹H NMR (400 MHz, DMSO-d6) δ 11.08 (s, 1H), 8.60 (s, 1H), 8.17 (s, |
| | | 1H, FA), 7.80 (s, 1H), 7.62 (d, J = 8.3 Hz, 1H), 7.27 (s, 2H), 6.78 (d, |
| | | J = 2.1 Hz, 1H), 6.68-6.61 (m, 1H), 5.05 (dd, J = 12.9, 5.3 Hz, 1H), |
| | | 4.08 (s, 3H), 3.84 (s, 6H), 3.73 (s, 4H), 3.56 (s, 2H), 3.53 (s, 3H), |
| | | 2.91-2.81 (m, 1H), 2.62-2.52 (m, 2H), 2.46-2.41 (m, 4H), 2.04- |
| | | 1.96 (m, 1H), 1.77-1.70 (m, 4H). |
| D416 | 680.4 | ¹H NMR (400 MHz, DMSO-d6) δ 10.96 (s, 1H), 8.59 (s, 1H), 8.18 (s, |
| | | 1H, FA), 7.80 (s, 1H), 7.36 (d, J = 8.1 Hz, 1H), 7.27 (s, 2H), 6.72- |
| | | 6.65 (m, 2H), 5.08 (dd, J = 13.3, 5.1 Hz, 1H), 4.35-4.13 (m, 2H), |
| | | 4.08 (s, 3H), 3.85 (s, 6H), 3.59-3.50 (m, 9H), 2.96-2.84 (m, 1H), |
| | | 2.64-2.53 (m, 2H), 2.48-2.36 (m, 4H), 2.02-1.94 (m, 1H), 1.76- |
| | | 1.69 (m, 4H). |
| D417 | 694.25 | ¹H NMR (400 MHz, DMSO-d6) δ 10.97 (s, 1H), 8.65 (s, 1H), 8.20 (s, |
| | | 1H FA), 7.83 (s, 1H), 7.36 (d, J = 8.1 Hz, 1H), 7.32 (s, 2H), 6.72- |
| | | 6.64 (m, 2H), 5.08 (dd, J = 13.3, 5.1 Hz, 1H), 4.38 (q, J = 7.3 Hz, |
| | | 2H), 4.31 (d, J = 16.6 Hz, 1H), 4.18 (d, J = 16.7 Hz, 1H), 3.85 (s, |
| | | 6H), 3.57 (d, J = 4.1 Hz, 6H), 3.53 (s, 3H), 2.98-2.84 (m, 1H), 2.70- |
| | | 2.52 (m, 2H), 2.49-2.42 (m, 3H), 2.37 (dd, J = 13.2, 4.6 Hz, 1H), |
| | | 2.03-1.94 (m, 1H), 1.74 (t, J = 5.5 Hz, 4H), 1.50 (t, J = 7.3 Hz, 3H). |
| D418 | 693.45 | ¹H NMR (400 MHz, DMSO-d6) δ 11.85 (d, J = 2.7 Hz, 1H), 11.09 (s, |
| | | 1H), 7.66 (d, J = 8.3 Hz, 1H), 7.17-7.10 (m, 2H), 6.78 (d, J = 2.1 |
| | | Hz, 1H), 6.70 (s, 2H), 6.65 (dd, J = 8.4, 2.1 Hz, 1H), 5.06 (dd, J = |
| | | 12.9, 5.4 Hz, 1H), 3.82 (d, J = 22.7 Hz, 12H), 3.55 (s, 3H), 2.95- |
| | | 2.83 (m, 2H), 2.82-2.66 (m, 2H), 2.64-2.53 (m, 4H), 2.01 (dd, J = |
| | | 9.4, 4.3 Hz, 1H), 1.87 (s, 6H). |
| D419 | 665.25 | ¹H NMR (300 MHz, DMSO-d6) δ 12.17 (s, 1H), 10.98 (s, 1H), 8.15 |
| | | (s, 1H, FA), 7.48 (s, 1H), 7.38-7.36 (m, 2H), 6.85 (s, 2H), 6.71- |
| | | 6.68 (m, 2H), 6.58-6.55 (m, 1H), 5.11-5.05 (m, 1H), 4.34-4.15 |
| | | (m, 2H), 3.85 (s, 6H), 3.60 (s, 3H), 3.58 (s, 6H), 2.97-2.89 (m, 1H), |
| | | 2.74-2.72 (m, 3H), 2.40-2.34 (m, 2H), 2.00-1.97 (m, 1H), 1.73 |
| | | (s, 4H), 1.35-1.24 (m, 1H). |
| D420 | 666.35 | ¹H NMR (400 MHz, DMSO-d6) δ 13.50 (s, 1H), 10.97 (s, 1H), 8.15 |
| | | (s, 1H), 7.77 (s, 1H), 7.38 (d, J = 8.0 Hz, 1H), 6.88 (s, 2H), 6.73- |
| | | 6.65 (m, 2H), 5.08 (dd, J = 13.3, 5.1 Hz, 1H), 4.31 (d, J = 16.6 Hz, |
| | | 1H), 4.18 (d, J = 16.5 Hz, 1H), 3.89 (s, 6H), 3.69 (s, 2H), 3.60 (s, |
| | | 4H), 3.55 (s, 3H), 2.96-2.84 (m, 1H), 2.64-2.55 (m, 4H), 2.42- |
| | | 2.22 (m, 2H), 1.98 (d, J = 12.9 Hz, 1H), 1.79 (s, 4H). |
| D421 | 680.45 | ¹H NMR (400 MHz, DMSO-d6) δ 10.97 (s, 1H), 8.45 (s, 1H), 8.18 (s, |
| | | 1H, FA), 7.47 (s, 1H), 7.37 (d, J = 8.0 Hz, 1H), 6.83 (s, 2H), 6.72- |
| | | 6.64 (m, 2H), 5.08 (dd, J = 13.2, 5.1 Hz, 1H), 4.31 (d, J = 16.6 Hz, |
| | | 1H), 4.18 (d, J = 16.6 Hz, 1H), 4.12 (s, 3H), 3.87 (s, 6H), 3.57 (s, |
| | | 3H), 3.56-3.53 (m, 6H), 2.95-2.86 (m, 1H), 2.63-2.56 (m, 1H), |
| | | 2.49-2.34 (m, 5H), 2.04-1.94 (m, 1H), 1.79-1.62 (m, 4H). |
| D422 | 679.25 | ¹H NMR (300 MHz, DMSO-d6) δ 11.99 (s, 1H), 10.99 (s, 1H), 9.05- |
| | | 8.75 (m, 1H, TFA), 7.51 (s, 1H), 7.41 (d, J = 8.8 Hz, 1H), 6.94 (s, |
| | | 2H), 6.71 (d, J = 5.7 Hz, 2H), 6.30 (s, 1H), 5.08 (dd, J = 13.2, 5.1 Hz, |
| | | 1H), 4.36-4.15 (m, 4H), 3.95 (s, 6H), 3.76 (s, 2H), 3.65-3.63 (m, |
| | | 2H), 3.62-3.60 (m, 3H), 3.38-3.33 (m, 2H), 3.18-3.05 (m, 2H), |
| | | 3.00-2.84 (m, 1H), 2.67-2.59 (m, 1H), 2.44-2.39 (m, 1H), 2.36 |
| | | (s, 3H), 2.13 (d, J = 13.3 Hz, 2H), 2.01 (d, J = 11.3 Hz, 3H). |
| D423 | 614.35 | ¹H NMR (400 MHz, DMSO-d6) δ 7.53 (d, J = 8.5 Hz, 1H), 7.31 (d, J = |
| | | 1.2 Hz, 1H), 7.05 (d, J = 8.3 Hz, 2H), 6.56 (s, 2H), 5.00 (dd, J = |
| | | 13.3, 5.1 Hz, 1H), 4.39-4.15 (m, 2H), 3.77 (d, J = 18.2 Hz, 8H), |
| | | 3.53 (s, 3H), 3.32 (t, J = 4.8 Hz, 4H), 2.94-2.81 (m, 1H), 2.79- |
| | | 2.67 (m, 4H), 2.65-2.55 (m, 1H), 2.43-2.28 (m, 4H), 2.03 (s, 3H), |
| | | 2.01-1.92 (m, 1H). |
| D424 | 671.4 | ¹H NMR (300 MHz, DMSO-d6) δ 8.56 (s, 1H), 8.24 (s, 1H, FA), 7.77 |
| | | (s, 1H), 7.43 (d, J = 8.4 Hz, 1H), 7.25 (s, 2H), 7.21 (d, J = 6.0 Hz, |
| | | 1H), 7.15 (d, 1H), 5.05 (dd, J = 13.2, 5.0 Hz, 1H), 4.34-4.20 (m, |
| | | 2H), 4.07 (s, 3H), 4.00 (d, J = 12.7 Hz, 1H), 3.86 (s, 6H), 3.83-3.77 |
| | | (m, 1H), 3.28-3.14 (m, 4H), 3.06-2.96 (m, 2H), 2.91-2.79 (m, |
| | | 1H), 2.66-2.55 (m, 1H), 2.43-2.20 (m, 1H), 2.00 (s, 1H), 1.26 (d, |
| | | J = 6.1 Hz, 6H). |
| D425 | 676.35 | ¹H NMR (400 MHz, DMSO-d6) δ 10.97 (s, 1H), 8.41 (d, J = 2.6 Hz, |
| | | 1H), 8.19 (d, J = 6.8 Hz, 1H), 8.16 (d, J = 2.6 Hz, 1H, FA), 7.36 (d, J = |
| | | 8.0 Hz, 1H), 6.90 (d, J = 33.7 Hz, 3H), 6.72-6.64 (m, 2H), 5.13- |
| | | 5.04 (m, 1H), 4.31 (d, J = 16.5 Hz, 1H), 4.18 (d, J = 16.6 Hz, 1H), |
| | | 3.87 (s, 6H), 3.60 (s, 3H), 3.54 (d, J = 15.6 Hz, 7H), 2.97-2.84 (m, |
| | | 1H), 2.63-2.54 (m, 1H), 2.45-2.31 (m, 4H), 1.98 (d, J = 12.6 Hz, |
| | | 1H), 1.71 (s, 4H). |
| D426 | 679.5 | ¹H NMR (300 MHz, DMSO-d6) δ 11.82 (s, 1H), 10.98 (s, 1H), 8.24 |
| | | FA (s, 1H), 7.37 (d, J = 8.0 Hz, 1H), 7.20-7.06 (m, 2H), 6.76-6.54 |
| | | (m, 4H), 5.09 (dd, J = 13.3, 5.0 Hz, 1H), 4.40-4.10 (m, 2H), 3.80 (s, |
| | | 6H), 3.60-3.54 (m, 9H), 3.00-2.83 (m, 2H), 2.62 (s, 1H), 2.40 (s, |
| | | 3H), 1.99 (s, 2H), 1.85 (s, 3H), 1.78-1.64 (m, 4H). |
| D427 | 614.35 | ¹H NMR (400 MHz, DMSO-d6) δ 10.97 (s, 1H), 7.42 (d, J = 8.4 Hz, |
| | | 1H), 7.31 (d, J = 1.2 Hz, 1H), 7.25 (dd, J = 8.5, 2.4 Hz, 1H), 7.14 (d, |
| | | J = 2.3 Hz, 1H), 6.59-6.52 (m, 2H), 5.09 (dd, J = 13.3, 5.1 Hz, 1H), |
| | | 4.39-4.16 (m, 2H), 3.81 (s, 6H), 3.62 (s, 2H), 3.54 (s, 3H), 3.22- |
| | | 3.11 (m, 4H), 2.98-2.84 (m, 1H), 2.72-2.56 (m, 5H), 2.46-2.36 |
| | | (m, 1H), 2.33 (s, 3H), 2.04 (s, 3H), 2.02-1.94 (m, 1H). |
| D428 | 666.35 | ¹H NMR (300 MHz, DMSO-d6) δ 10.97 (s, 1H), 8.36 (s, 1H), 8.15 (d, |
| | | J = 0.9 Hz, 1H, FA), 7.89 (s, 1H), 7.37 (d, J = 8.1 Hz, 1H), 7.12 (s, |
| | | 1H), 6.93 (s, 2H), 6.69 (d, J = 7.7 Hz, 2H), 5.08 (dd, J = 13.2, 5.1 Hz, |
| | | 1H), 4.43-4.14 (m, 2H), 3.86 (s, 6H), 3.64-3.57 (m, 6H), 3.44 (s, |
| | | 5H), 2.99-2.84 (m, 2H), 2.68-2.60 (m, 1H), 2.45-2.32 (m, 2H), |
| | | 2.05-1.91 (m, 1H), 1.81-1.68 (m, 4H). |
| D429 | 627.35 | ¹H NMR (400 MHz, DMSO-d6) δ 10.97 (s, 1H), 8.50 (d, J = 1.2 Hz, |
| | | 1H), 8.14 (d, J = 1.1 Hz, 1H), 7.38 (d, J = 8.0 Hz, 1H), 7.18 (s, 2H), |
| | | 6.68 (d, J = 7.7 Hz, 2H), 5.08 (dd, J = 13.3, 5.1 Hz, 1H), 4.42-4.14 |
| | | (m, 2H), 3.88 (s, 6H), 3.77 (s, 2H), 3.60 (s, 4H), 3.56 (s, 4H), 2.97- |
| | | 2.84 (m, 1H), 2.69 (s, 3H), 2.64-2.55 (m, 2H), 2.39 (td, J = 13.1, |
| | | 4.4 Hz, 1H), 2.02-1.93 (m, 1H), 1.82 (s, 4H). |
| D430 | 654.3 | ¹H NMR (300 MHz, DMSO-d6) δ 11.09 (s, 1H), 7.66 (d, J = 8.2 Hz, |
| | | 1H), 7.53 (s, 1H), 6.82 (d, J = 2.1 Hz, 1H), 6.73-6.54 (m, 3H), 5.06 |
| | | (dd, J = 12.8, 5.3 Hz, 1H), 4.14-3.94 (m, 5H), 3.85 (s, 6H), 3.49- |
| | | 3.46 (m, 5H), 3.14-2.97 (m, 2H), 2.96-2.70 (m, 2H), 2.68-2.58 |
| | | (m, 1H), 2.36-2.17 (m, 2H), 2.14-1.95 (m, 7H). |
| D431 | 665.35 | ¹H NMR (300 MHz, DMSO-d6) δ 10.99 (s, 1H), 8.94 (s, 1H, TFA), |
| | | 7.50 (d, J = 6.0 Hz, 1H), 7.41 (d, J = 8.9 Hz, 1H), 7.04 (d, J = 4.0 Hz, |
| | | 1H), 6.92 (s, 2H), 6.87 (dd, J = 10.2, 5.1 Hz, 2H), 6.75-6.66 (m, |
| | | 2H), 5.08 (dd, J = 13.3, 5.1 Hz, 1H), 4.39-4.12 (m, 4H), 3.95 (s, |
| | | 6H), 3.76 (s, 2H), 3.70 (s, 2H), 3.43-3.31 (m, 5H), 3.17-3.11 (m, |
| | | 2H), 3.00-2.82 (m, 1H), 2.63 (s, 1H), 2.44-2.30 (m, 1H), 2.19- |
| | | 2.08 (m, 2H), 2.06-1.96 (m, 3H). |
| D432 | 664.3 | ¹H NMR (300 MHz, Methanol-d4) δ 8.30 (d, J = 2.6 Hz, 1H), 8.20- |
| | | 8.13 (m, 1H), 7.72 (d, J = 7.7 Hz, 1H), 7.10-6.61 (m, 3H), 6.26 (dd, |
| | | J = 7.6, 2.0 Hz, 1H), 5.67 (d, J = 1.8 Hz, 1H), 5.25 (dd, J = 12.5, 5.3 |
| | | Hz, 1H), 4.43 (s, 2H), 4.17-3.96 (m, 10H), 3.72 (s, 3H), 3.67 (s, |
| | | 2H), 3.51-3.45 (m, 2H), 2.99-2.76 (m, 2H), 2.73-2.49 (m, 1H), |
| | | 2.52-2.46 (m, 2H), 2.36-2.23 (m, 1H). |
| D433 | 666.25 | ¹H NMR (300 MHz, DMSO-d6) δ 14.27 (s, 1H), 10.98 (s, 1H), 8.24 |
| | | (s, 1H, FA), 8.15 (s, 1H), 7.61 (s, 1H), 7.38 (d, J = 8.3 Hz, 1H), 6.90 |
| | | (s, 2H), 6.74-6.64 (m, 2H), 5.08 (dd, J = 13.2, 5.1 Hz, 1H), 4.25 |
| | | (dd, 2H), 3.90 (s, 6H), 3.74 (s, 2H), 3.61 (d, J = 6.9 Hz, 7H), 2.98- |
| | | 2.84 (m, 1H), 2.79-2.54 (m, 5H), 2.42-2.32 (m, 1H), 2.03-1.93 |
| | | (m, 1H), 1.81 (s, 4H). |
| D434 | 666.25 | ¹H NMR (300 MHz, MeOD) δ 8.85-8.49 (m, 1H), 7.85 (d, J = 1.9 |
| | | Hz, 1H), 7.42 (d, J = 8.2 Hz, 1H), 7.17-7.09 (m, 2H), 6.89 (d, J = |
| | | 2.2 Hz, 1H), 6.81 (dd, J = 8.2, 2.3 Hz, 1H), 5.14 (dd, J = 13.2, 5.1 |
| | | Hz, 1H), 4.49-4.31 (m, 4H), 4.04 (s, 6H), 3.85 (s, 2H), 3.78 (s, 3H), |
| | | 3.73 (s, 2H), 3.56 (d, J = 12.7 Hz, 2H), 3.24 (t, J = 11.9, 11.9 Hz, |
| | | 2H), 3.02-2.86 (m, 1H), 2.85-2.72 (m, 1H), 2.59-2.41 (m, 1H), |
| | | 2.34-2.22 (m, 2H), 2.21-2.04 (m, 3H). |
| D435 | 665.35 | ¹H NMR (400 MHz, DMSO-d6) δ 10.96 (s, 1H), 8.23 (s, 1H, FA), |
| | | 7.61 (d, J = 3.2 Hz, 1H), 7.36 (d, J = 8.0 Hz, 1H), 7.05 (d, J = 7.7 Hz, |
| | | 1H), 7.03 (d, J = 3.2 Hz, 1H), 6.88 (d, J = 7.7 Hz, 1H), 6.77 (s, 2H), |
| | | 6.70-6.67 (m, 2H), 5.08 (dd, J = 13.3, 5.1 Hz, 1H), 4.34-4.14 (m, |
| | | 2H), 3.85 (s, 6H), 3.56 (s, 3H), 3.51-3.50 (m, 6H), 2.98-2.83 (m, |
| | | 1H), 2.68-2.57 (m, 1H), 2.48-2.22 (m, 5H), 2.06-1.91 (m, 1H), |
| | | 1.72-1.70 (m, 4H). |
| D436 | 614.3 | ¹H NMR (300 MHz, Methanol-d4) δ 7.71 (d, J = 8.5 Hz, 1H), 7.60 (s, |
| | | 1H), 7.39 (d, J = 2.2 Hz, 1H), 7.27 (dd, J = 8.6, 2.3 Hz, 1H), 6.65 (s, |
| | | 2H), 6.52 (s, 1H), 5.09 (dd, J = 12.3, 5.4 Hz, 1H), 3.91 (d, 8H), 3.58 |
| | | (d, J = 18.3 Hz, 7H), 2.99-2.89 (m, 4H), 2.87-2.66 (m, 3H), 2.21 |
| | | (s, 3H), 2.18-2.07 (m, 1H). |
| D437 | 693.4 | ¹H NMR (300 MHz, Methanol-d4) 7.41 (d, J = 8.2 Hz, 1H), 6.99- |
| | | 6.78 (m, 2H), 6.70 (s, 2H), 5.86 (s, 1H), 5.29-5.05 (m, 1H), 4.52- |
| | | 4.29 (m, 2H), 4.22-3.98 (m, 5H), 3.91 (s, 6H), 3.74 (s, 4H), 3.21- |
| | | 2.72 (m, 6H), 2.66-2.40 (m, 1H), 2.35 (s, 3H), 2.27-2.15 (m, 4H), |
| | | 2.15-1.94 (m, 4H). |
| D438 | 595.3 | ¹H NMR (400 MHz, DMSO-d6 with a drop of D₂O) δ 8.15 (s, 1H, |
| | | FA), 8.05 (d, J = 2.7 Hz, 1H), 7.82 (dd, J = 2.7, 1.3 Hz, 1H), 7.70- |
| | | 7.59 (m, 3H), 6.82 (s, 2H), 5.10 (dd, J = 13.3, 5.1 Hz, 1H), 4.42 (dd, |
| | | 2H), 3.86 (s, 6H), 3.66 (s, 2H), 3.65-3.56 (m, 2H), 3.53 (s, 3H), |
| | | 3.34 (s, 1H), 3.28 (d, J = 7.7 Hz, 2H), 2.97-2.84 (m, 1H), 2.66- |
| | | 2.56 (m, 1H), 2.44-2.35 (m, 1H), 2.10 (s, 3H), 2.07-1.95 (m, 1H). |
| D439 | 631.3 | ¹H NMR (300 MHz, Methanol-d4) δ 8.25 (s, 1H), 8.11 (s, 1H), 7.79 |
| | | (d, J = 1.2 Hz, 1H), 7.67 (dd, J = 7.9, 1.5 Hz, 1H), 7.57 (d, J = 7.9 |
| | | Hz, 1H), 7.09-6.61 (m, 3H), 5.17 (dd, J = 13.3, 5.2 Hz, 1H), 4.61- |
| | | 4.42 (m, 2H), 4.23 (s, 2H), 4.16 (t, J = 8.8 Hz, 2H), 4.00 (s, 6H), 3.97- |
| | | 3.85 (m, 2H), 3.77-3.63 (m, 4H), 3.03-2.75 (m, 2H), 2.61-2.40 |
| | | (m, 1H), 2.29-2.13 (m, 1H). |
| D440 | 595.3 | ¹H NMR (400 MHz, Methanol-d4) δ 7.99-7.90 (m, 1H), 7.84-7.74 |
| | | (m, 2H), 7.72-7.49 (m, 2H), 6.91 (d, J = 2.6 Hz, 2H), 5.20-5.11 |
| | | (m, 1H), 4.55-4.37 (m, 6H), 4.34-4.22 (m, 2H), 4.00 (s, 6H), 3.95- |
| | | 3.84 (m, 1H), 3.66 (d, J = 6.7 Hz, 3H), 2.98-2.85 (m, 1H), 2.81 (s, |
| | | 1H), 2.54-2.40 (m, 1H), 2.20 (d, J = 5.0 Hz, 4H). |
| D441 | 631.5 | ¹H NMR (400 MHz, Methanol-d4) δ 8.32-8.07 (m, 2H), 7.77 (d, J = |
| | | 7.8 Hz, 1H), 7.70-7.53 (m, 2H), 7.07-6.67 (m, 3H), 5.15 (dd, J = |
| | | 13.3, 5.1 Hz, 1H), 4.62-4.39 (m, 6H), 4.35-4.23 (m, 2H), 4.17- |
| | | 3.83 (m, 7H), 3.68 (s, 3H), 2.98-2.84 (m, 1H), 2.78 (d, J = 17.4 Hz, |
| | | 1H), 2.48 (qd, J = 13.1, 4.7 Hz, 1H), 2.22-2.14 (m, 1H). |
| D442 | 609.5 | ¹H NMR (300 MHz, Methanol-d4) δ 8.56 (s, FA, 1H), 7.96 (d, 1H), |
| | | 7.85-7.79 (m, 2H), 7.71-7.63 (m, 1H), 7.58 (d, 1H), 6.90 (s, 2H), |
| | | 5.23-5.11 (m, 1H), 4.52 (d, 2H), 4.41 (s, 2H), 4.00 (s, 6H), 3.69 (s, |
| | | 3H), 3.63-3.50 (m, 2H), 3.48-3.36 (m, 3H), 3.00-2.73 (m, 2H), |
| | | 2.61-2.39 (m, 2H), 2.31-2.11 (m, 5H). |
| D443 | 623.35 | ¹H NMR (300 MHz, DMSO-d6) δ 11.01 (s, 1H), 8.06 (d, J = 2.7 Hz, |
| | | 1H), 7.89-7.75 (m, 1H), 7.69-7.54 (m, 3H), 6.82 (s, 2H), 5.11 (dd, |
| | | J = 13.2, 5.1 Hz, 1H), 4.61-4.25 (m, 2H), 3.86 (s, 6H), 3.58 (s, 2H), |
| | | 3.54 (s, 3H), 3.02-2.84 (m, 1H), 2.84-2.70 (m, 2H), 2.67-2.53 |
| | | (m, 2H), 2.48-2.35 (m, 1H), 2.33-2.21 (m, 2H), 2.10 (s, 3H), 2.06- |
| | | 1.95 (m, 1H), 1.92-1.79 (m, 2H), 1.69-1.53 (m, 2H). |
| D444 | 659.3 | ¹H NMR (300 MHz, DMSO-d6) δ 11.01 (s, 1H), 8.40 (.1.0 FA, d, J = |
| | | 2.6 Hz, 1H), 8.26-8.05 (m, 2H), 7.75-7.51 (m, 3H), 7.20-6.69 |
| | | (m, 3H), 5.11 (dd, J = 13.2, 5.1 Hz, 1H), 4.61-4.21 (m, 2H), 3.87 (s, |
| | | 6H), 3.60 (s, 3H), 3.56 (s, 2H), 3.00-2.83 (m, 1H), 2.82-2.69 (m, |
| | | 2H), 2.68-2.53 (m, 2H), 2.48-2.32 (m, 1H), 2.32-2.18 (m, 2H), |
| | | 2.08-1.93 (m, 1H), 1.84 (d, 2H), 1.70-1.49 (m, 2H). |
| D445 | 707.4 | ¹H NMR (300 MHz, DMSO-d6) δ 11.58 (s, 1H), 11.08 (s, 1H), 8.28- |
| | | 8.13 (m, 1H, FA), 7.64 (d, J = 8.3 Hz, 1H), 6.99 (d, J = 2.6 Hz, 1H), |
| | | 6.77 (d, J = 2.1 Hz, 1H), 6.64 (dd, J = 8.4, 2.2 Hz, 1H), 6.51 (s, 2H), |
| | | 5.06 (dd, J = 12.7, 5.2 Hz, 1H), 3.74 (d, J = 8.4 Hz, 10H), 3.58 (d, J = |
| | | 3.6 Hz, 5H), 2.94-2.83 (m, 1H), 2.65-2.55 (m, 3H), 2.47-2.38 |
| | | (m, 3H), 2.17 (s, 3H), 2.07-1.97 (m, 1H), 1.79-1.67 (m, 4H), 1.51 |
| | | (s, 3H). |
| D446 | 707.4 | ¹H NMR (300 MHz, DMSO-d6) δ 11.08 (s, 1H), 10.85 (s, 1H), 7.65 |
| | | (d, J = 8.3 Hz, 1H), 6.79 (d, J = 2.1 Hz, 1H), 6.70-6.62 (m, 1H), |
| | | 6.59 (s, 2H), 5.83 (s, 1H), 5.06 (dd, J = 12.7, 5.3 Hz, 1H), 4.00 (s, |
| | | 3H), 3.89-3.72 (m, 10H), 3.68 (s, 2H), 3.00-2.78 (m, 1H), 2.70- |
| | | 2.53 (m, 6H), 2.27 (s, 3H), 2.14(s, 3H), 2.09-1.96 (m, 1H), 1.81 (s, 4H). |
| D447 | 666.4 | ¹H NMR (300 MHz, Methanol-d4) δ 8.52 (br s, 0.2H, FA), 7.84 (d, J = |
| | | 1.2 Hz, 1H), 7.64 (s, 1H), 7.48 (d, J = 8.2 Hz, 1H), 7.30 (s, 1H), |
| | | 7.10 (s, 2H), 6.95 (d, J = 2.2 Hz, 1H), 6.87 (dd, J = 8.2, 2.2 Hz, 1H), |
| | | 5.20 (dd, 1H), 4.47 (d, J = 5.4 Hz, 2H), 4.41 (s, 2H), 4.07 (s, 6H), |
| | | 3.86-3.71 (m, 8H), 3.38-3.28 (m, 3H) 3.18-2.80 (m, 2H), 2.62- |
| | | 2.54 (m, 1H), 2.36-2.10 (m, 5H). |
| D448 | 599.35 | ¹H NMR (400 MHz, DMSO-d6) δ 10.99 (s, 1H), 8.19 (s, 1H, FA), |
| | | 8.06 (d, J = 2.6 Hz, 1H), 7.81 (d, J = 2.3 Hz, 1H), 7.54 (s, 1H), 7.51 |
| | | (d, J = 7.8 Hz, 1H), 7.44 (dd, J = 7.8, 1.6 Hz, 1H), 6.81 (s, 2H), 5.10 |
| | | (dd, J = 13.3, 5.2 Hz, 1H), 4.46-4.23 (m, 2H), 3.84 (s, 6H), 3.72 (d, |
| | | J = 4.9 Hz, 2H), 3.53 (s, 3H), 3.46 (s, 2H), 3.07 (s, 2H), 2.96-2.85 |
| | | (m, 1H), 2.64-2.56 (m, 4H), 2.39 (dd, J = 13.3, 4.7 Hz, 1H), 2.10 (s, |
| | | 3H), 2.03-1.96 (m, 1H), 1.79 (q, J = 7.5 Hz, 2H). |
| D449 | 585.35 | ¹H NMR (400 MHz, DMSO-d6) δ 10.99 (s, 1H), 8.19 (s, 1H, FA), |
| | | 8.06 (d, J = 2.6 Hz, 1H), 7.81 (d, J = 2.3 Hz, 1H), 7.54 (s, 1H), 7.51 |
| | | (d, J = 7.8 Hz, 1H), 7.44 (dd, J = 7.8, 1.6 Hz, 1H), 6.81 (s, 2H), 5.10 |
| | | (dd, J = 13.3, 5.2 Hz, 1H), 4.46-4.23 (m, 2H), 3.84 (s, 6H), 3.72 (d, |
| | | J = 4.9 Hz, 2H), 3.53 (s, 3H), 3.46 (s, 2H), 3.07 (s, 2H), 2.96-2.85 |
| | | (m, 1H), 2.64-2.56 (m, 4H), 2.39 (dd, J = 13.3, 4.7 Hz, 1H), 2.10 (s, |
| | | 3H), 2.03-1.96 (m, 1H), 1.79 (q, J = 7.5 Hz, 2H). |
| D450 | 609.30 | ¹H NMR (400 MHz, DMSO-d6) δ 8.22 (s, 1H, FA), 8.04 (d, J = 2.7 |
| | | Hz, 1H), 7.86-7.80 (m, 1H), 7.70-7.57 (m, 3H), 6.82 (s, 2H), 5.09 |
| | | (dd, J = 13.3, 5.1 Hz, 1H), 4.55-4.31 (m, 2H), 3.86 (s, 6H), 3.66 (s, |
| | | 2H), 3.54 (s, 3H), 3.43 (d, J = 6.8 Hz, 2H), 3.27 (d, J = 6.8 Hz, 2H), |
| | | 2.99-2.81 (m, 1H), 2.74-2.58 (m, 1H), 2.46-2.32 (m, 1H), 2.10 |
| | | (s, 3H), 2.07-1.97 (m, 1H), 1.51 (s, 3H). |
| D451 | 538.25 | ¹H NMR (300 MHz, DMSO-d6) δ 11.02 (s, 1H), 8.48 (d, J = 2.4 Hz, |
| | | 1H), 8.22 (s, 1H), 7.61 (d, J = 7.9 Hz, 1H), 7.54-7.45 (m, 2H), 7.18- |
| | | 6.74 (m, 3H), 5.14 (dd, J = 13.2, 5.0 Hz, 1H), 4.43 (dd, 2H), 3.79 |
| | | (s, 6H), 3.62 (s, 3H), 3.04-2.91 (m, 1H), 2.67-2.59 (m, 1H), 2.47- |
| | | 2.34 (m, 1H), 2.07-1.97 (m, 1H). |
| D452 | 600.25 | ¹H NMR (400 MHz, DMSO-d6) δ 10.98 (s, 1H), 9.39 (s, 1H), 8.16 (d, |
| | | J = 2.7 Hz, 1H, FA), 7.88 (dd, J = 2.7, 1.3 Hz, 1H), 7.49 (d, J = 8.4 |
| | | Hz, 1H), 7.31 (dd, J = 8.4, 2.4 Hz, 1H), 7.26 (d, J = 2.3 Hz, 1H), 6.96 |
| | | (s, 2H), 5.10 (dd, J = 13.3, 5.1 Hz, 1H), 4.36 (d, J = 16.9 Hz, 1H), |
| | | 4.31 (d, J = 4.2 Hz, 2H), 4.23 (d, J = 16.9 Hz, 1H), 3.96 (s, 6H), 3.88 |
| | | (d, J = 13.1 Hz, 2H), 3.55 (s, 3H), 3.47 (d, J = 12.0 Hz, 2H), 3.33- |
| | | 3.20 (m, 2H), 3.12 (t, J = 12.4 Hz, 2H), 2.98-2.84 (m, 1H), 2.60 (d, |
| | | J = 17.7 Hz, 1H), 2.43-2.32 (m, 1H), 2.11 (s, 3H), 2.04-1.94 (m, 1H). |
| D453 | 609.50 | ¹H NMR (400 MHz, DMSO-d6) δ 11.00 (s, 1H), 8.25 (s, 1H, FA salt), |
| | | 8.05 (d, J = 2.7 Hz, 1H), 7.82 (d, J = 2.6 Hz, 1H), 7.63-7.56 (m, |
| | | 3H), 6.82 (s, 2H), 5.10 (dd, J = 13.3, 5.2 Hz, 1H), 4.37 (dd, 2H), 3.86 |
| | | (s, 6H), 3.62-3.58 (m, 2H), 3.53 (s, 3H), 3.15-3.13 (m, 1H), 2.96- |
| | | 2.89 (m, 2H), 2.86-2.83 (m, 2H), 2.62-2.58 (m, 1H), 2.41-2.35 |
| | | (m, 1H), 2.09 (s, 3H), 2.03-1.98 (m, 1H), 1.11 (d, J = 5.9 Hz, 3H). |
| D454 | 706.4 | ¹H NMR (400 MHz, DMSO-d6) δ 10.89 (s, 1H), 8.28 (s, 1H), 7.82 (s, |
| | | 1H), 7.30 (d, J = 8.0 Hz, 1H), 6.96 (s, 1H), 6.85 (s, 2H), 6.62 (d, J = |
| | | 8.2 Hz, 2H), 5.73-5.62 (m, 1H), 5.59-5.48 (m, 1H), 5.00 (dd, J = |
| | | 13.2, 5.1 Hz, 1H), 4.36 (d, J = 6.0 Hz, 2H), 4.28-4.07 (m, 2H), 3.79 |
| | | (s, 6H), 3.61-3.49 (m, 8H), 2.91-2.77 (m, 1H), 2.54 (d, J = 3.7 Hz, |
| | | 3H), 2.37-2.23 (m, 1H), 1.96-1.86 (m, 1H), 1.68 (t, J = 6.6 Hz, 4H), |
| | | 1.60 (d, J = 6.3 Hz, 3H). |
| D455 | 593.25 | ¹H NMR (400 MHz, Methanol-d4) δ 8.16 (d, J = 2.6 Hz, 1H), 8.11- |
| | | 8.05 (m, 1H), 7.38 (d, J = 8.3 Hz, 1H), 6.99-6.78 (m, 3H), 6.76 (s, |
| | | 2H), 5.12 (dd, J = 13.3, 5.1 Hz, 1H), 4.45-4.31 (m, 5H), 3.99 (q, J = |
| | | 5.5, 4.1 Hz, 2H), 3.82 (s, 6H), 3.67 (s, 3H), 2.97-2.83 (m, 1H), 2.83- |
| | | 2.72 (m, 1H), 2.56-2.40 (m, 1H), 2.21-2.11 (m, 1H). |
| D456 | 666.5 | ¹H NMR (400 MHz, Methanol-d4) δ 8.52 (s, 0.67H, FA), 7.93 (d, J = |
| | | 2.2 Hz, 1H), 7.39 (d, J = 8.2 Hz, 1H), 7.28 (s, 1H), 7.22-7.15 (m, |
| | | 3H), 6.86 (d, J = 2.2 Hz, 1H), 6.81-6.74 (m, 1H), 5.16-5.07 (m, |
| | | 1H), 4.45-4.30 (m, 4H), 3.97 (s, 6H), 3.75 (s, 4H), 3.63 (s, 3H), 3.39- |
| | | 3.36 (m, 2H), 3.28-3.21 (m, 2H), 2.96-2.83 (m, 1H), 2.82-2.72 |
| | | (m, 1H), 2.56-2.40 (m, 1H), 2.19-2.02 (m, 5H). |
| D457 | 597.35 | ¹H NMR (400 MHz, DMSO-d6) δ 10.99 (s, 1H), 9.67-9.52 (m, 1H, |
| | | TFA salt), 8.17-8.10 (m, 1H), 7.88-7.78 (m, 2H), 7.71-7.64 (m, |
| | | 1H), 7.62-7.56 (m, 1H), 6.98-6.91 (m, 2H), 6.71-6.62 (m, 5.7 |
| | | Hz, 2H), 5.17-5.06 (m, 1H), 4.49-4.41 (m, 1H), 4.41-4.27 (m, |
| | | 3H), 4.27-4.22 (m, 2H), 4.16-4.15 (m, 2H), 3.95 (s, 6H), 3.54 (s, |
| | | 3H), 2.97-2.85 (m, 1H), 2.61 (d, J = 17.1 Hz, 1H), 2.43-2.35 (m, |
| | | 2H), 2.10 (s, 3H), 2.04-1.97 (m, 2H). |
| D458 | 613.35 | ¹H NMR (400 MHz, DMSO-d6) δ 8.08 (s, 1H), 7.89 (s, 1H), 7.79 (d, |
| | | J = 8.0 Hz, 2H), 7.71 (d, J = 7.9 Hz, 1H), 6.92 (s, 2H), 5.09 (dd, J = |
| | | 13.3, 5.1 Hz, 1H), 4.86-4.57 (m, 4H), 4.57-4.45 (m, 3H), 4.40 (d, |
| | | J = 18.1 Hz, 1H), 3.94 (s, 6H), 3.54 (s, 3H), 2.97-2.83 (m, 1H), |
| | | 2.71-2.59 (m, 1H), 2.47-2.33 (m, 1H), 2.16-2.01 (s, 4H). |
| D459 | 526.2 | ¹H NMR (400 MHz, DMSO-d6) δ 10.97 (s, 1H), 7.87 (d, J = 2.7 Hz, |
| | | 1H), 7.69 (dd, J = 2.7, 1.3 Hz, 1H), 7.46 (t, J = 8.7 Hz, 3H), 7.35 (dd, |
| | | J = 8.4, 2.4 Hz, 1H), 7.25 (d, J = 2.4 Hz, 1H), 7.05 (d, J = 8.8 Hz, |
| | | 2H), 5.11 (dd, J = 13.3, 5.1 Hz, 1H), 4.46-4.19 (m, 2H), 3.50 (s, |
| | | 3H), 3.35 (s, 8H), 2.98-2.85 (m, 1H), 2.60 (d, J = 17.0 Hz, 1H), |
| | | 2.43-2.32 (m, 1H), 2.07 (s, 3H), 2.04-1.95 (m, 1H). |
| D460 | 540.25 | ¹H NMR (400 MHz, DMSO-d6) δ 7.99 (d, J = 2.7 Hz, 1H), 7.75 (d, J = |
| | | 2.6 Hz, 1H), 7.57 (d, J = 7.7 Hz, 2H), 7.43 (dd, J = 11.4, 7.8 Hz, |
| | | 3H), 7.27 (dd, J = 8.5, 2.4 Hz, 1H), 7.18 (s, 1H), 5.09 (dd, J = 13.3, |
| | | 5.1 Hz, 1H), 4.44-4.17 (m, 2H), 3.66 (s, 3H), 3.52 (s, 3H), 3.24 (s, |
| | | 3H), 2.97-2.84 (m, 1H), 2.77-2.64 (m, 1H), 2.67 (s, 4H), 2.45- |
| | | 2.33 (m, 1H), 2.09 (s, 3H), 2.04-1.95 (m, 1H). |
| D461 | 641.25 | ¹H NMR (300 MHz, DMSO-d6) δ 11.02 (s, 1H), 9.00 (s, 1H, TFA), |
| | | 8.49 (s, 1H), 7.47 (d, J = 8.8 Hz, 1H), 7.32 (s, 2H), 6.77 (dt, J = 4.0, |
| | | 2.0 Hz, 2H), 5.14 (dd, J = 13.2, 5.1 Hz, 1H), 4.46-4.21 (m, 4H), |
| | | 4.02 (s, 6H), 3.81 (s, 2H), 3.71 (s, 2H), 3.62 (s, 3H), 3.43 (d, J = 12.6 |
| | | Hz, 2H), 3.18 (t, J = 11.4 Hz, 2H), 3.07-2.89 (m, 1H), 2.67 (d, J = |
| | | 17.2 Hz, 1H), 2.56-2.31 (m, 4H), 2.19 (d, J = 14.0 Hz, 2H), 2.12- |
| | | 1.98 (m, 3H), 0.08 (s, 1H). |
| D462 | 696.5 | ¹H NMR (400 MHz, DMSO-d6 with a drop of D2O) δ 8.75 (s, 1H), |
| | | 8.26 (s, 1H, FA), 7.89 (s, 1H), 7.39 (d, J = 8.1 Hz, 1H), 7.24 (s, 2H), |
| | | 6.74-6.67 (m, 2H), 5.05 (dd, J = 13.3, 5.1 Hz, 1H), 4.37-4.15 (m, |
| | | 5H), 3.88 (s, 6H), 3.80 (s, 2H), 3.61 (s, 4H), 3.55 (s, 3H), 2.95-2.82 |
| | | (m, 1H), 2.81-2.57 (m, 5H), 2.45-2.31 (m, 1H), 2.06-1.95 (m, |
| | | 1H), 1.85 (t, J = 5.5 Hz, 4H). |
| D463 | 654.35 | ¹H NMR (400 MHz, Methanol-d4) δ 8.90 (s, 1H), 8.28 (d, J = 1.1 Hz, |
| | | 1H), 7.87 (d, J = 1.7 Hz, 1H), 7.73 (dd, J = 7.9, 1.7 Hz, 1H), 7.61 (d, |
| | | J = 7.9 Hz, 1H), 7.48 (d, J = 8.5 Hz, 1H), 7.39-7.31 (m, 2H), 7.15 |
| | | (s, 1H), 5.95-5.80 (m, 1H), 5.71-5.54 (m, 1H), 5.13 (dd, J = 13.3, |
| | | 5.1 Hz, 1H), 4.52 (d, J = 6.4 Hz, 2H), 4.48-4.34 (m, 2H), 4.01 (t, J = |
| | | 5.3 Hz, 2H), 3.59-3.52 (m, 2H), 3.42-3.38 (m, 2H), 3.36-3.32 |
| | | (m, 1H), 3.28-3.20 (m, 1H), 2.95-2.83 (m, 1H), 2.82-2.73 (m, 1H), |
| | | 2.56-2.42 (m, 1H), 2.23-2.13 (m, 1H), 1.73 (dd, J = 6.5, 1.6 Hz, 3H). |
| D464 | 704.1 | ¹H NMR (300 MHz, DMSO-d6) δ 11.04 (s, 1H), 8.22 (s, 1H), 8.08- |
| | | 7.95 (m, 3H), 7.92 (s, 1H), 7.79 (s, 1H), 7.67 (s, 2H), 7.05 (s, 1H), |
| | | 5.82-5.66 (m, 1H), 5.66-5.51 (m, 1H), 5.18 (dd, J = 13.2, 5.1 Hz, |
| | | 1H), 4.68-4.46 (m, 3H), 4.41 (d, J = 6.0 Hz, 2H), 3.51 (s, 2H), 3.02- |
| | | 2.84 (m, 8H), 2.62 (d, J = 17.0 Hz, 1H), 2.49-2.34 (m, 1H), 2.05 |
| | | (dd, J = 12.7, 6.4 Hz, 1H), 1.65 (dd, J = 6.3, 1.4 Hz, 3H). |
| D465 | 680.4 | ¹H NMR (400 MHz, Methanol-d4) δ 8.06 (s, 1H), 7.86 (s, 1H), 7.68- |
| | | 7.60 (m, 2H), 7.52 (dd, J = 7.9, 1.8 Hz, 1H), 7.27 (d, J = 8.2 Hz, 1H), |
| | | 6.79-6.73 (m, 2H), 6.67 (dd, J = 8.2, 2.3 Hz, 1H), 5.82-5.68 (m, |
| | | 1H), 5.61-5.47 (m, 1H), 5.02 (dd, J = 13.3, 5.2 Hz, 1H), 4.39 (d, J = |
| | | 6.3 Hz, 2H), 4.34-4.20 (m, 2H), 3.71 (s, 2H), 3.59 (s, 4H), 2.87- |
| | | 2.74 (m, 1H), 2.72-2.65 (m, 1H), 2.62-2.51 (m, 4H), 2.45-2.31 |
| | | (m, 1H), 2.10-2.03 (m, 1H), 1.87-1.81 (m, 4H), 1.63 (dd, J = 6.5, |
| | | 1.5 Hz, 3H). |
| D466 | 640.4 | ¹H NMR (300 MHz, DMSO-d6) δ 11.05 (s, 1H), 8.32 (s, 1H), 8.02- |
| | | 7.76 (m, 4H), 7.61-7.49 (m, 1H), 7.43-7.27 (m, 2H), 7.16 (s, 1H), |
| | | 5.90-5.76 (m, 1H), 5.74-5.60 (m, 1H), 5.18 (dd, J = 13.2, 5.1 Hz, |
| | | 1H), 4.88-4.57 (m, 1H), 4.54-4.38 (m, 3H), 4.36-4.24 (m, 1H), |
| | | 4.10-3.56 (m, 3H), 3.32-3.14 (m, 3H), 3.07-2.90 (m, 2H), 2.75- |
| | | 2.62 (m, 3H), 2.52-2.38 (m, 1H), 2.14-2.04 (m, 1H), 1.78-1.70 |
| | | (m, 3H). |
| D467 | 719.45 | ¹H NMR (400 MHz, DMSO-d6) δ 11.18 (d, J = 6.1 Hz, 1H), 10.96 (s, |
| | | 1H), 8.94 (s, 1H, TFA), 7.40 (d, J = 8.9 Hz, 1H), 7.11 (s, 1H), 6.89 |
| | | (s, 2H), 6.70 (h, J = 2.3 Hz, 2H), 6.35 (s, 1H), 5.67-5.55 (m, 1H), |
| | | 5.45-5.29 (m, 1H), 5.15 (d, J = 5.5 Hz, 2H), 5.07 (dd, J = 13.2, 5.1 |
| | | Hz, 1H), 4.32 (d, J = 16.7 Hz, 1H), 4.25 (d, J = 4.6 Hz, 2H), 4.19 (d, |
| | | J = 16.6 Hz, 1H), 3.93 (s, 6H), 3.75 (s, 2H), 3.64 (s, 2H), 3.41-3.33 |
| | | (m, 2H), 3.11 (q, J = 11.1 Hz, 2H), 2.90 (ddd, J = 17.5, 13.4, 5.4 Hz, |
| | | 1H), 2.70-2.52 (m, 1H), 2.39 (dd, J = 13.2, 8.5 Hz, 1H), 2.34 (s, |
| | | 3H), 2.12 (d, J = 13.9 Hz, 2H), 2.04-1.94 (m, 3H), 1.62 (dd, J = 6.6, |
| | | 1.6 Hz, 3H). |
| D468 | 679.5 | ¹H NMR (400 MHz, DMSO-d6) δ 11.19 (d, J = 6.2 Hz, 1H), 10.98 (s, |
| | | 1H), 9.42 (s, 1H, TFA), 7.50 (d, J = 8.4 Hz, 1H), 7.32 (dd, J = 8.4, |
| | | 2.4 Hz, 1H), 7.29-7.20 (m, 1H), 7.12 (d, J = 6.0 Hz, 1H), 6.90 (s, |
| | | 2H), 6.36 (s, 1H), 5.66-5.54 (m, 1H), 5.45-5.29 (m, 1H), 5.15 (d, |
| | | J = 5.6 Hz, 1H), 5.10 (dd, J = 13.3, 5.1 Hz, 1H), 4.36 (d, J = 16.8 Hz, |
| | | 3H), 4.23 (d, J = 16.9 Hz, 1H), 3.94 (s, 6H), 3.89 (d, J = 12.9 Hz, |
| | | 2H), 3.74 (d, J = 7.0 Hz, 1H), 3.54-3.46 (m, 2H), 3.29 (d, J = 11.7 |
| | | Hz, 2H), 3.14 (t, J = 12.1 Hz, 2H), 2.91 (ddd, J = 17.6, 13.6, 5.4 Hz, |
| | | 1H), 2.60 (d, J = 17.0 Hz, 1H), 2.46-2.33 (m, 1H), 2.34 (s, 3H), |
| | | 2.03-1.95 (m, 1H), 1.81-1.59 (m, 3H). |
| D469 | 654.25 | ¹H NMR (400 MHz, DMSO-d6) δ 10.96 (s, 1H), 8.35 (s, 1H), 7.88 (s, |
| | | 1H), 7.41 (d, J = 8.4 Hz, 1H), 7.29-7.20 (m, 1H), 7.20-7.06 (m, |
| | | 2H), 6.92 (s, 2H), 5.09 (dd, J = 13.3, 5.1 Hz, 1H), 4.37-4.15 (m, |
| | | 2H), 3.86 (s, 6H), 3.66-3.51 (m, 2H), 3.43 (s, 3H), 3.11-2.85 (m, |
| | | 5H), 2.70-2.55 (m, 3H), 2.43-2.31 (m, 1H), 2.05-1.93 (m, 1H), |
| | | 1.39-1.14 (m, 6H). |
| D470 | 666.735 | |
| D471 | 666.45 | ¹H NMR (300 MHz, Methanol-d4) δ 9.62 (s, 1H), 8.72 (d, J = 6.3 Hz, |
| | | 1H), 8.15 (d, J = 7.0 Hz, 1H), 8.05 (s, 1H), 7.93 (d, J = 6.3 Hz, 1H), |
| | | 7.36 (d, J = 2.4 Hz, 1H), 6.90 (s, 2H), 6.79 (dd, J = 7.1, 2.4 Hz, 1H), |
| | | 4.96 (d, J = 9.1 Hz, 1H), 4.47 (s, 2H), 4.27 (s, 2H), 4.15 (s, 2H), 3.99 |
| | | (s, 6H), 3.76 (s, 3H), 3.63 (d, J = 13.1 Hz, 2H), 3.25 (t, J = 12.2 Hz, |
| | | 2H), 2.94-2.70 (m, 2H), 2.27 (dt, J = 28.7, 13.5 Hz, 6H). |
| D472 | 667.20 | ¹H NMR (300 MHz, Methanol-d4) δ 9.55 (d, J = 0.8 Hz, 1H), 8.70 (d, |
| | | J = 5.8 Hz, 1H), 8.56 (d, J = 5.0 Hz, 1H), 7.77 (s, 1H), 7.64 (d, J = |
| | | 5.8, 0.9 Hz, 1H), 7.28 (d, J = 4.9 Hz, 1H), 6.85 (s, 2H), 4.82 (dd, J = |
| | | 12.6, 5.4 Hz, 1H), 4.20 (s, 2H), 4.06-3.91 (m, 10H), 3.72 (s, 3H), |
| | | 3.06 (d, J = 27.6 Hz, 4H), 2.95-2.65 (m, 2H), 2.43-2.27 (m, 1H), |
| | | 2.20 (s, 1H), 2.14-1.99 (m, 4H). |
| D473 | 667.20 | ¹H NMR (400 MHz, DMSO-d6) δ 10.93 (s, 1H), 9.48 (s, 1H), 9.02 (d, |
| | | J = 15.8 Hz, 1H), 8.73 (dd, J = 16.7, 7.0 Hz, 2H), 8.40 (s, 1H), 8.10 |
| | | (s, 1H), 7.91 (s, 1H), 7.60 (d, J = 5.7 Hz, 1H), 6.88 (s, 2H), 4.84- |
| | | 4.73 (m, 1H), 4.30 (d, J = 4.6 Hz, 2H), 4.02 (s, 2H), 3.91 (s, 8H), |
| | | 3.62 (s, 3H), 3.40 (d, J = 12.2 Hz, 2H), 3.21-3.02 (m, 2H), 2.82 (s, |
| | | 1H), 2.55 (d, J = 3.7 Hz, 1H), 2.25-2.11 (m, 3H), 2.08-1.91 (m, 3H). |
| D474 | 677.45 | ¹H NMR (300 MHz, DMSO-d6) δ 10.98 (s, 1H), 9.45 (s, 1H), 8.74 (d, |
| | | J = 5.7 Hz, 1H), 7.89 (s, 1H), 7.59 (d, J = 5.6 Hz, 1H), 7.38 (d, J = |
| | | 8.1 Hz, 1H), 6.76 (s, 2H), 6.70-6.61 (m, 2H), 4.80-4.67 (m, 1H), |
| | | 4.33 (s, 2H), 3.83 (s, 6H), 3.67-3.53 (m, 9H), 3.03-2.88 (m, 2H), |
| | | 2.78-2.64 (m, 2H), 2.60-2.53 (m, 4H), 1.82-1.69 (m, 4H). |
| D475 | 748.35 | ¹H NMR (400 MHz, Methanol-d4) δ 7.42 (d, J = 8.2 Hz, 1H), 7.21 (s, |
| | | 1H), 7.03 (d, J = 3.2 Hz, 1H), 6.90-6.85 (m, 3H), 6.82-6.78 (m, |
| | | 2H), 5.14 (dd, J = 13.2, 5.1 Hz, 1H), 4.64-4.49 (m, 2H), 4.45-4.34 |
| | | (m, 4H), 4.25-4.13 (m, 2H), 3.97 (s, 6H), 3.87-3.71 (m, 4H), 3.66 |
| | | (s, 3H), 3.62-3.46 (m, 5H), 3.44-3.38 (m, 4H), 3.16-3.05 (m, |
| | | 1H), 2.98-2.86 (m, 1H), 2.85-2.75 (m, 1H), 2.56-2.42 (m, 1H), |
| | | 2.32-2.06 (m, 5H). |
| D476 | 693.2 | ¹H NMR (400 MHz, DMSO-d6) δ 11.90 (s, 1H), 11.08 (s, 1H), 8.25 |
| | | (s, 1H, FA), 7.63 (d, J = 8.3 Hz, 1H), 7.43 (s, 1H), 6.84-6.75 (m, |
| | | 3H), 6.65 (dd, J = 8.5, 2.2 Hz, 1H), 6.29 (s, 1H), 5.05 (dd, J = 12.9, |
| | | 5.4 Hz, 1H), 3.84 (s, 6H), 3.73 (s, 4H), 3.58 (s, 3H), 3.52 (s, 2H), |
| | | 2.94-2.85 (m, 1H), 2.62-2.55 (m, 2H), 2.44-2.37 (m, 3H), 2.37- |
| | | 2.31 (m, 4H), 2.06-1.96 (m, 1H), 1.73 (t, J = 5.2 Hz, 4H). |

### Example 85—Preparation of Compounds 0011-0016

In analogy to the procedures described in the examples above, compounds D011-D016 were prepared using the appropriate starting materials.

| Compound No. | LCMS | ¹H NMR |
|---|---|---|
| DD11 | 785.35 | ¹H NMR (300 MHz, DMSO) δ 11.13 (s, 1H), 8.20 (s, FA, 1H), 8.09 |
| | | (d, J = 8.3 Hz, 1H), 7.88-7.80 (m, 2H), 7.74 (s, 1H), 7.56-7.48 (m, |
| | | 1H), 7.47-7.39 (m, 1H), 7.39-7.35 (m, 1H), 7.34-7.23 (m, 2H), |
| | | 6.73 (s, 2H), 5.12 (dd, J = 12.9, 5.4 Hz, 1H), 5.06-4.91 (m, 1H), |
| | | 3.81 (s, 6H), 3.70 (s, 2H), 3.58-3.50 (m, 1H), 3.00-2.81 (m, 4H), |
| | | 2.66-2.53 (m, 1H), 2.49-2.38 (m, 4H), 2.35-2.18 (m, 6H), 2.14- |
| | | 1.99 (m, 3H), 1.86-1.75 (m, 2H), 1.72-1.61 (m, 4H), 1.60-1.49 |
| | | (m, 3H), 1.27-1.07 (m, 2H). |
| DD12 | 519.45 | ¹H NMR (400 MHz, DMSO-d6) δ 10.97 (s, 1H), 7.37 (d, J = 8.0 Hz, |
| | | 1H), 7.28 (t, J = 8.3 Hz, 1H), 6.72-6.64 (m, 4H), 5.08 (dd, J = 13.3, |
| | | 5.1 Hz, 1H), 4.35-4.12 (m, 2H), 3.79 (s, 6H), 3.64 (s, 2H), 3.57 (s, |
| | | 4H), 2.98-2.84 (m, 1H), 2.64-2.55 (m, 5H), 2.45-2.33 (m, 1H), |
| | | 2.02-1.94 (m, 1H), 1.79-1.72 (m, 4H). |
| DD13 | 676.35 | ¹H NMR (400 MHz, DMSO-d6) δ 10.98 (s, 1H), 8.30 (dd, J = 8.2, 1.4 |
| | | Hz, 1H), 7.71 (dd, J = 7.4, 1.4 Hz, 1H), 7.58 (t, J = 7.7 Hz, 1H), 7.45 |
| | | (d, J = 7.7 Hz, 1H), 7.38 (d, J = 8.0 Hz, 1H), 6.69 (d, J = 4.3 Hz, 4H), |
| | | 6.56 (d, J = 7.6 Hz, 1H), 5.09 (dd, J = 13.3, 5.1 Hz, 1H), 4.36-4.13 |
| | | (m, 2H), 3.82 (s, 7H), 3.60 (d, J = 4.4 Hz, 7H), 3.53 (s, 3H), 2.98- |
| | | 2.84 (m, 1H), 2.64-2.55 (m, 2H), 2.38 (dd, J = 13.2, 4.6 Hz, 2H), |
| | | 2.03-1.94 (m, 1H), 1.75 (t, J = 5.4 Hz, 4H). |
| DD14 | 479.30 | ¹H NMR (300 MHz, Methanol-d4) δ 8.52 (s, 0.48H, FA), 7.53-7.40 |
| | | (m, 2H), 7.40-7.32 (m, 2H), 6.78 (d, J = 8.4 Hz, 2H) 5.15 (dd, J = |
| | | 13.3, 5.1 Hz, 1H), 4.52-4.35 (m, 2H), 4.27 (s, 2H), 3.93 (s, 6H), |
| | | 3.62-3.39 (m, 4H), 3.30-3.18 (m, 4H), 3.12-2.73 (m, 2H), 2.62- |
| | | 2.41 (m, 1H), 2.26-2.12 (m, 1H). |
| DD15 | 652.30 | ¹H NMR (300 MHz, DMSO-d6) δ 12.70 (s, 1H), 10.97 (s, 1H), 8.22- |
| | | 8.13 (m, 3H), 7.36 (d, J = 8.0 Hz, 1H), 6.74-6.34 (m, 4H), 5.07 (dd, |
| | | J = 13.6, 5.2 Hz, 1H), 4.34-4.14 (m, 2H), 3.88 (s, 6H), 3.65-3.57 |
| | | (m, 6H), 2.94-2.86 (m, 1H), 2.67-2.59 (m, 1H), 2.47-2.26 (m, |
| | | 5H), 2.04-1.93 (m, 1H), 1.84-1.59 (m, 4H). |
| DD16 | 518.15 | ¹H NMR (300 MHz, DMSO-d6) δ 10.98 (s, 1H), 8.47 (d, J = 7.9 Hz, |
| | | 1H), 7.78 (d, J = 7.5 Hz, 1H), 7.66-7.53 (m, 1H), 7.45 (m, J = 8.4 |
| | | Hz, 1H), 7.37-7.08 (m, 2H), 6.74 (m, J = 7.5, 0.9 Hz, 1H), 5.11 (dd, |
| | | J = 13.2, 5.1 Hz, 1H), 4.44-4.13 (m, 2H), 4.00 (s, 1H), 3.89-3.67 |
| | | (m, 2H), 3.52 (s, 3H), 3.00-2.91 (m, 3H), 2.63 (m, 1H), 2.45-2.23 |
| | | (m, 1H), 2.11-1.94 (m, 1H), 1.95-1.81 (m, 2H), 1.71-1.61 (m, 2H). |

### Example 86—BRD9 Bromodomain TR-FRET Competition Binding Assay

This example demonstrates the ability of the compounds of the disclosure to biochemically inhibit BRD9 bromodomain in a competition binding assay.

### Procedure:

His-Flag-BRD9 (P133-K239; Swiss Prot Q9H8M2; SEQ ID NO:1 mgsshhhhhhenlyfq/gdykddddkgslevlfqg/PAENESTPIQQLLEHFLRQLQRKDPHGFFAFPVTDAIAPGYSMII KHPMDFGTMKDKIVANEYKSVTEFKADFKLMCDNAMTYNRPDTVYYKLAKKILHAGFKMMSK) was cloned, expressed, purified, and then treated with TEV protease. Cleaved His tag was removed by purification. The binding of a biotinylated small molecule ligand of BRD9 was assessed via the LANCE® TR-FRET platform (PerkinElmer), and the compounds were assayed for inhibitory activity against this interaction.

A mixture of biotinylated-ligand and SureLight™ Allophycocyanin-Streptavidin (APC-SA, PerkinElmer AD0201) in 50 mM HEPES (pH 7.4), 50 mM NaCl, 1 mM TCEP (pH 7), 0.01% (v/v) Tween-20, 0.01% (w/v) bovine serum albumin was added to a white 384-well PerkinElmer Proxiplate Plus plate. DMSO or 3-fold serially diluted compounds were then added to the Proxiplate followed by addition of Flag-BRD9. After a 10-minute incubation at room temperature, Eu-W1024 anti-FLAG (PerkinElmer, AD0273) was added. The final reaction mixture that contained 3.75 nM biotinylated ligand, 3 nM Flag-BRD9, 7.5 nM SureLight™ Allophycocyanin-Streptavidin, and 0.2 nM Eu-W1024 anti-FLAG was incubated at room temperature for 90 minutes.

### Results:

The plates were then read on a PerkinElmer Envision plate reader to determine the ratio of emission at 665 nm over 615 nm. Data was normalized to a DMSO control (100%) and a no protein control (0%) and then fit to a four parameter, non-linear curve fit to calculate IC₅₀ (μM) values as shown in Table 4. As shown by the results in Table 4, a number of compounds of the present disclosure exhibit an IC₅₀ value of <1 μM for BRD9 binding, indicating their affinity for targeting BRD9.

TABLE 4
Bromodomain 9 (BRD9) TR-FRET
Binding of Compounds of the Disclosure

| | Bromodomain |
|---|---|
| Compound | TR-FRET BRD9 |
| No. | IC₅₀ (nM) |
| D1 | +++ |
| D2 | ++++ |
| D3 | ++++ |
| D4 | ++++ |
| D5 | ++++ |
| D6 | ++++ |
| D7 | ++++ |
| D8 | ++++ |
| D9 | ++++ |
| D10 | +++ |
| D11 | +++ |
| D12 | ++++ |
| D13 | ++++ |
| D14 | ++++ |
| D15 | ++++ |
| D16 | NT |
| D17 | NT |
| D18 | NT |
| D19 | NT |
| D20 | NT |
| D21 | NT |
| D22 | ++++ |
| D23 | ++++ |
| D24 | NT |
| D25 | ++++ |
| D26 | +++ |
| D27 | ++++ |
| D28 | NT |
| D29 | NT |
| D30 | ++++ |
| D31 | ++++ |
| D32 | ++++ |
| D33 | ++++ |
| D34 | ++++ |
| D35 | ++++ |
| D36 | ++++ |
| D37 | ++++ |
| D38 | ++++ |
| D39 | ++++ |
| D40 | ++++ |
| D41 | ++++ |
| D42 | NT |
| D43 | NT |
| D44 | NT |
| D45 | NT |
| D46 | NT |
| D47 | NT |
| D48 | NT |
| D49 | NT |
| D50 | NT |
| D51 | NT |
| D52 | ++++ |
| D53 | ++++ |
| D54 | +++ |
| D55 | +++ |
| D56 | ++++ |
| D57 | +++ |
| D58 | ++++ |
| D59 | ++++ |
| D60 | ++++ |
| D61 | ++++ |
| D62 | +++ |
| D63 | +++ |
| D64 | +++ |
| D65 | +++ |
| D66 | +++ |
| D67 | ++++ |
| D68 | ++++ |
| D69 | ++++ |
| D70 | +++ |
| D71 | ++++ |
| D72 | ++++ |
| D73 | +++ |
| D74 | ++++ |
| D75 | NT |
| D76 | NT |
| D77 | NT |
| D78 | NT |
| D79 | NT |
| D80 | NT |
| D81 | NT |
| D82 | NT |
| D83 | NT |
| D84 | +++ |
| D85 | +++ |
| D86 | +++ |
| D87 | ++++ |
| D88 | ++++ |
| D89 | ++++ |
| D90 | ++++ |
| D91 | +++ |
| D92 | ++++ |
| D93 | ++++ |
| D94 | +++ |
| D95 | +++ |
| D96 | ++++ |
| D97 | ++++ |
| D98 | ++ |
| D99 | +++ |
| D100 | ++++ |
| D101 | ++++ |
| D102 | ++++ |
| D103 | +++ |
| D104 | +++ |
| D105 | +++ |
| D106 | +++ |
| D107 | +++ |
| D108 | ++++ |
| D109 | ++++ |
| D110 | +++ |
| D111 | ++++ |
| D112 | ++++ |
| D113 | ++++ |
| D114 | ++++ |
| D115 | +++ |
| D116 | ++++ |
| D117 | ++++ |
| D118 | ++++ |
| D119 | ++++ |
| D120 | +++ |
| D121 | +++ |
| D122 | ++++ |
| D123 | ++++ |
| D124 | ++++ |
| D125 | ++++ |
| D126 | +++ |
| D127 | ++++ |
| D128 | +++ |
| D129 | ++++ |
| D130 | ++++ |
| D131 | ++++ |
| D132 | ++++ |
| D133 | ++++ |
| D134 | +++ |
| D135 | +++ |
| D136 | ++++ |
| D137 | ++++ |
| D138 | ++++ |
| D139 | ++++ |
| D140 | ++++ |
| D141 | +++ |
| D142 | +++ |
| D143 | ++++ |
| D144 | +++ |
| D145 | +++ |
| D146 | ++++ |
| D147 | +++ |
| D148 | +++ |
| D149 | ++++ |
| D150 | ++++ |
| D151 | ++++ |
| D152 | ++++ |
| D153 | +++ |
| D154 | ++++ |
| D155 | ++++ |
| D156 | ++++ |
| D157 | +++ |
| D158 | ++++ |
| D159 | ++++ |
| D160 | ++++ |
| D161 | ++++ |
| D162 | +++ |
| D163 | ++++ |
| D164 | ++++ |
| D165 | +++ |
| D166 | ++++ |
| D167 | ++++ |
| D168 | ++++ |
| D169 | ++++ |
| D170 | +++ |
| D171 | +++ |
| D172 | ++++ |
| D173 | +++ |
| D174 | +++ |
| D175 | +++ |
| D176 | ++++ |
| D177 | ++++ |

“+” indicates inhibitory effect of ≥1000 nM;
“++” indicates inhibitory effect of ≥100 nM;
“+++” indicates inhibitory effect of ≥10 nM;
“++++” indicates inhibitory effect of <10 nM;
“NT” indicates not tested

### Example 87—SYO1 BRD9 NanoLuc Degradation Assay

This example demonstrates the ability of the compounds of the disclosure to degrade a Nanoluciferase-BRD9 fusion protein in a cell-based degradation assay.

### Procedure:

A stable SYO-1 cell line expressing 3×FLAG-NLuc-BRD9 was generated. On day 0 cells were seeded in 30 μL media into each well of 384-well cell culture plates. The seeding density was 8000 cells/well. On day 1, cells were treated with 30 nL DMSO or 30 nL of 3-fold serially DMSO-diluted compounds (10 points in duplicates with 1 μM as final top dose). Subsequently plates were incubated for 6 hours in a standard tissue culture incubator and equilibrated at room temperature for 15 minutes. Nanoluciferase activity was measured by adding 15 μL of freshly prepared Nano-Glo Luciferase Assay Reagent (Promega N1130), shaking the plates for 10 minutes and reading the bioluminescence using an EnVision reader.

### Results:

The Inhibition % was calculated using the following formula: % Inhibition=100×(Lum_{HC}−Lum_{Sample})/(Lum_{HC}−Lum_{LC}). DMSO treated cells are employed as High Control (HC) and 1 μM of a known BRD9 degrader standard treated cells are employed as Low Control (LC). The data was fit to a four parameter, non-linear curve fit to calculate IC₅₀ (μM) values as shown in Table 5A, Table 5B, and Table 5C. As shown by the results in Table 5A, Table 5B, and Table 5C, a number of compounds of the present disclosure exhibit an IC₅₀ value of <1 μM for the degradation of BRD9, indicating their use as compounds for reducing the levels and/or activity of BRD9 and their potential for treating BRD9-related disorders.

TABLE 5A
SYO1 Bromodomain 9-NanoLuc Degradation
by Compounds of the Disclosure

| Compound | SYO1 BRD9-NanoLuc |
|---|---|
| No. | degradation IC₅₀ (nM) |
| D1 | ++++ |
| D2 | +++ |
| D3 | ++++ |
| D4 | +++ |
| D5 | +++ |
| D6 | ++++ |
| D7 | +++ |
| D8 | + |
| D9 | ++++ |
| D10 | ++++ |
| D11 | ++++ |
| D12 | ++++ |
| D13 | ++++ |
| D14 | ++++ |
| D15 | ++++ |
| D16 | ++++ |
| D17 | ++++ |
| D18 | ++++ |
| D19 | ++++ |
| D20 | ++++ |
| D21 | + |
| D22 | +++ |
| D23 | ++++ |
| D24 | +++ |
| D25 | ++ |
| D26 | + |
| D27 | +++ |
| D28 | ++ |
| D29 | +++ |
| D30 | +++ |
| D31 | +++ |
| D32 | +++ |
| D33 | ++++ |
| D34 | ++++ |
| D35 | ++++ |
| D36 | ++ |
| D37 | ++++ |
| D38 | ++++ |
| D39 | ++++ |
| D40 | ++++ |
| D41 | +++ |
| D42 | ++++ |
| D43 | ++ |
| D44 | ++++ |
| D45 | ++++ |
| D46 | ++++ |
| D47 | ++++ |
| D48 | +++ |
| D49 | + |
| D50 | ++++ |
| D51 | ++++ |
| D52 | ++++ |
| D53 | ++++ |
| D54 | +++ |
| D55 | ++ |
| D56 | ++++ |
| D57 | ++++ |
| D58 | ++++ |
| D59 | ++++ |
| D60 | ++++ |
| D61 | +++ |
| D62 | ++ |
| D63 | +++ |
| D64 | ++ |
| D65 | ++ |
| D66 | ++ |
| D67 | ++++ |
| D68 | ++ |
| D69 | ++++ |
| D70 | +++ |
| D71 | ++++ |
| D72 | ++++ |
| D73 | ++++ |
| D74 | ++ |
| D75 | ++++ |
| D76 | ++++ |
| D77 | ++ |
| D78 | +++ |
| D79 | ++ |
| D80 | ++++ |
| D81 | ++++ |
| D82 | +++ |
| D83 | ++ |
| D84 | + |
| D85 | ++ |
| D86 | ++ |
| D87 | +++ |
| D88 | +++ |
| D89 | ++++ |
| D90 | +++ |
| D91 | +++ |
| D92 | ++++ |
| D93 | +++ |
| D94 | +++ |
| D95 | ++ |
| D96 | +++ |
| D97 | +++ |
| D98 | ++ |
| D99 | +++ |
| D100 | ++++ |
| D101 | ++ |
| D102 | +++ |
| D103 | +++ |
| D104 | ++ |
| D105 | ++ |
| D106 | ++ |
| D107 | +++ |
| D108 | ++++ |
| D109 | +++ |
| D110 | +++ |
| D111 | +++ |
| D112 | ++ |
| D113 | ++++ |
| D114 | +++ |
| D115 | ++ |
| D116 | +++ |
| D117 | ++ |
| D118 | +++ |
| D119 | +++ |
| D120 | +++ |
| D121 | +++ |
| D122 | ++++ |
| D123 | ++++ |
| D124 | ++++ |
| D125 | +++ |
| D126 | ++ |
| D127 | ++ |
| D128 | ++++ |
| D129 | ++++ |
| D130 | ++++ |
| D131 | ++++ |
| D132 | ++++ |
| D133 | +++ |
| D134 | +++ |
| D135 | ++ |
| D136 | ++ |
| D137 | +++ |
| D138 | +++ |
| D139 | ++ |
| D140 | +++ |
| D141 | ++ |
| D142 | +++ |
| D143 | ++++ |
| D144 | +++ |
| D145 | +++ |
| D146 | +++ |
| D147 | +++ |
| D148 | ++ |
| D149 | +++ |
| D150 | +++ |
| D151 | +++ |
| D152 | ++++ |
| D153 | +++ |
| D154 | +++ |
| D155 | +++ |
| D156 | +++ |
| D157 | ++++ |
| D158 | +++ |
| D159 | +++ |
| D160 | +++ |
| D161 | +++ |
| D162 | +++ |
| D163 | +++ |
| D164 | +++ |
| D165 | +++ |
| D166 | +++ |
| D167 | ++++ |
| D168 | ++++ |
| D169 | +++ |
| D170 | ++++ |
| D171 | ++++ |
| D172 | +++ |
| D173 | ++++ |
| D174 | ++ |
| D175 | +++ |
| D176 | ++++ |
| D177 | +++ |

“+” indicates inhibitory effect of ≥1000 nM;
“++” indicates inhibitory effect of ≥100 nM;
“+++” indicates inhibitory effect of ≥10 nM;
“++++” indicates inhibitory effect of <10 nM;
“NT” indicates not tested

TABLE 5B
SYO1 Bromodomain 9-NanoLuc Degradation
by Compounds of the Disclosure

| Compound | SYO1 BRD9-NanoLuc |
|---|---|
| No. | degradation IC₅₀ (nM) |
| D178 | ++++ |
| D179 | +++ |
| D180 | ++++ |
| D181 | ++ |
| D182 | +++ |
| D183 | ++ |
| D184 | ++++ |
| D185 | ++++ |
| D186 | ++++ |
| D187 | ++++ |
| D188 | ++++ |
| D189 | ++++ |
| D190 | +++ |
| D191 | ++++ |
| D192 | ++ |
| D193 | ++ |
| D194 | ++++ |
| D195 | +++ |
| D196 | +++ |
| D197 | ++++ |
| D198 | ++++ |
| D199 | ++++ |
| D200 | +++ |
| D201 | ++++ |
| D202 | ++++ |
| D203 | ++++ |
| D204 | ++++ |
| D205 | ++++ |
| D206 | ++++ |
| D207 | ++++ |
| D208 | ++++ |
| D209 | ++ |
| D210 | +++ |
| D211 | ++++ |
| D212 | +++ |
| D213 | ++++ |
| D214 | ++++ |
| D215 | ++++ |
| D216 | ++++ |
| D217 | ++++ |
| D218 | ++++ |
| D219 | ++++ |
| D220 | ++++ |
| D221 | ++++ |
| D222 | ++++ |
| D223 | ++++ |
| D224 | ++++ |
| D225 | ++++ |
| D226 | ++++ |
| D227 | ++++ |
| D228 | ++++ |
| D229 | ++++ |
| D230 | ++++ |
| D231 | ++ |
| D232 | +++ |
| D233 | ++ |
| D234 | +++ |
| D235 | ++++ |
| D236 | ++++ |
| D237 | ++++ |
| D238 | ++++ |
| D239 | ++++ |
| D240 | ++++ |
| D241 | ++++ |
| D242 | ++++ |
| D243 | ++++ |
| D244 | ++++ |
| D245 | +++ |
| D246 | ++++ |
| D247 | ++++ |
| D248 | +++ |
| D249 | +++ |
| D250 | ++++ |
| D251 | ++++ |
| D252 | ++++ |
| D253 | ++++ |
| D254 | ++++ |
| D255 | ++++ |
| D256 | ++++ |
| D257 | ++++ |
| D258 | ++++ |
| D259 | ++++ |
| D260 | ++++ |
| D261 | ++++ |
| D262 | ++++ |
| D263 | ++++ |
| D264 | +++ |
| D265 | ++ |
| D266 | +++ |
| D267 | +++ |
| D268 | ++++ |
| D269 | ++++ |
| D270 | +++ |
| D271 | ++++ |
| D272 | ++++ |
| D273 | ++++ |
| D274 | ++++ |
| D275 | ++++ |
| D276 | +++ |
| D277 | ++++ |
| D278 | +++ |
| D279 | ++++ |
| D280 | ++++ |
| D281 | +++ |
| D282 | ++ |
| D283 | ++ |
| D284 | +++ |
| D285 | ++ |
| D286 | +++ |
| D287 | ++++ |
| D288 | ++++ |
| D289 | ++++ |
| D290 | ++++ |
| D291 | ++++ |
| D292 | ++ |
| D293 | +++ |
| D294 | ++ |
| D295 | ++ |
| D296 | ++ |
| D297 | ++++ |
| D298 | ++++ |
| D299 | ++++ |
| D300 | ++++ |
| D301 | ++++ |
| D302 | ++++ |
| D303 | +++ |
| D304 | ++++ |
| D305 | ++ |
| D306 | ++++ |
| D307 | ++++ |
| D308 | ++++ |
| D309 | +++ |
| D310 | ++++ |
| D311 | +++ |
| D312 | ++++ |
| D313 | ++++ |
| D314 | +++ |
| D315 | ++++ |
| D316 | ++++ |
| D317 | +++ |
| D318 | ++++ |
| D319 | ++++ |
| D320 | ++++ |
| D321 | ++++ |
| D322 | ++++ |
| D323 | ++++ |
| D324 | ++++ |
| D325 | ++++ |
| D326 | ++++ |
| D327 | ++++ |
| D328 | ++++ |
| D329 | ++++ |
| D330 | ++++ |
| D331 | ++++ |
| D332 | ++++ |
| D333 | ++++ |
| D334 | + |
| D335 | ++++ |
| D336 | ++++ |
| D337 | ++++ |
| D338 | ++++ |
| D339 | ++++ |
| D340 | ++++ |
| D341 | ++++ |
| D342 | + |
| D343 | ++++ |
| D344 | ++++ |
| D345 | ++++ |
| D346 | ++++ |
| D347 | ++++ |
| D348 | ++++ |
| D349 | ++++ |
| D350 | ++ |
| D351 | + |
| D352 | + |
| D353 | ++++ |
| D354 | ++++ |
| D355 | + |
| D356 | ++++ |
| D357 | ++++ |
| D358 | ++++ |
| D359 | ++++ |
| D360 | ++++ |
| D361 | ++++ |
| D362 | ++++ |
| D363 | ++++ |
| D364 | ++ |
| D365 | +++ |
| D366 | ++++ |
| D367 | ++++ |
| D368 | ++++ |
| D369 | ++++ |
| D370 | ++++ |
| D371 | ++++ |
| DD1 | + |
| DD2 | ++ |
| DD3 | + |
| DD4 | ++++ |
| DD5 | +++ |
| DD6 | +++ |
| DD7 | ++++ |
| DD8 | ++++ |
| DD9 | ++++ |
| DD10 | ++ |

“+” indicates inhibitory effect of ≥1000 nM;
“++” indicates inhibitory effect of ≥100 nM;
“+++” indicates inhibitory effect of ≥10 nM;
“++++” indicates inhibitory effect of <10 nM;
“NT” indicates not tested

TABLE 5C
SYO1 Bromodomain 9-NanoLuc Degradation by
Compounds of the Disclosure

| Compound | SYO1 BRD9-NanoLuc |
|---|---|
| No. | degradation IC₅₀ (nM) |
| D372 | ++++ |
| D373 | ++++ |
| D374 | ++++ |
| D375 | ++++ |
| D376 | ++++ |
| D377 | ++++ |
| D378 | ++++ |
| D379 | ++++ |
| D380 | +++ |
| D381 | ++++ |
| D382 | ++++ |
| D383 | + |
| D384 | ++++ |
| D385 | ++++ |
| D386 | ++++ |
| D387 | ++++ |
| D388 | ++++ |
| D389 | + |
| D390 | + |
| D391 | ++ |
| D392 | +++ |
| D393 | +++ |
| D394 | + |
| D395 | ++++ |
| D396 | ++++ |
| D397 | ++++ |
| D398 | ++++ |
| D399 | ++++ |
| D400 | ++++ |
| D401 | ++++ |
| D402 | ++++ |
| D403 | ++++ |
| D404 | ++++ |
| D405 | ++++ |
| D406 | ++++ |
| D407 | ++++ |
| D408 | ++++ |
| D409 | ++++ |
| D410 | ++++ |
| D411 | ++++ |
| D412 | ++++ |
| D413 | ++++ |
| D414 | ++++ |
| D415 | ++++ |
| D416 | ++++ |
| D417 | ++++ |
| D418 | ++++ |
| D419 | ++++ |
| D420 | ++++ |
| D421 | ++++ |
| D422 | ++++ |
| D423 | ++++ |
| D424 | ++++ |
| D425 | ++++ |
| D426 | ++++ |
| D427 | ++++ |
| D428 | ++++ |
| D429 | + |
| D430 | ++++ |
| D431 | ++++ |
| D432 | +++ |
| D433 | ++++ |
| D434 | ++++ |
| D435 | + |
| D436 | ++++ |
| D437 | + |
| D438 | ++++ |
| D439 | ++++ |
| D440 | ++++ |
| D441 | ++++ |
| D442 | ++++ |
| D443 | ++++ |
| D444 | ++++ |
| D445 | + |
| D446 | + |
| D447 | ++ |
| D448 | ++++ |
| D449 | +++ |
| D450 | ++++ |
| D451 | +++ |
| D452 | ++++ |
| D453 | ++++ |
| D454 | ++++ |
| D455 | ++++ |
| D456 | ++++ |
| D457 | ++++ |
| D458 | ++++ |
| D459 | ++++ |
| D460 | ++++ |
| D461 | +++ |
| D462 | ++++ |
| D463 | ++++ |
| D464 | + |
| D465 | ++++ |
| D466 | ++++ |
| D467 | + |
| D468 | + |
| D469 | NT |
| D470 | NT |
| D471 | ++++ |
| D472 | + |
| D473 | + |
| D474 | + |
| D475 | +++ |
| D476 | ++++ |
| DD11 | + |
| DD12 | + |
| DD13 | +++ |
| DD14 | + |
| DD15 | +++ |
| DD16 | +++ |

“+” indicates inhibitory effect of ≥1000 nM;
“++” indicates inhibitory effect of ≥100 nM;
“+++” indicates inhibitory effect of ≥10 nM;
“++++” indicates inhibitory effect of <10 nM;
“NT” indicates not tested

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A compound having the structure of Formula I: 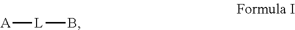
wherein
A is a BRD9 binding moiety;
B is a degradation moiety; and
L has the structure of Formula II: 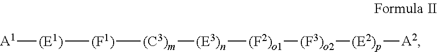
wherein
A¹ is a bond between the linker and A;
A² is a bond between B and the linker;
each of m, n, o1, o2, and p is, independently, 0 or 1;
each of E¹ and E² is, independently, O, S, NR^{N}, optionally substituted C₁₋₁₀ alkylene, optionally substituted C₂₋₁₀ alkenylene, optionally substituted C₂₋₁₀ alkynylene, optionally substituted C₂-C₁₀ polyethylene glycol, or optionally substituted C₁₋₁₀ heteroalkylene;
E³ is optionally substituted C₁-C₆ alkylene, optionally substituted C₁-C₆ heteroalkylene, O, S, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;
C₃ is carbonyl, thiocarbonyl, sulphonyl, or phosphoryl; and
each of F¹, F², and F³ is, independently, optionally substituted C₃-C₁₀ carbocyclylene, optionally substituted C₂₋₁₀ heterocyclylene, optionally substituted C₆-C₁₀ arylene, or optionally substituted C₂-C₉ heteroarylene,
or a pharmaceutically acceptable salt thereof.

2.-327. (canceled)

328. A method of treating an infection in a subject in need thereof, the method comprising intrathecally administering to the subject an effective amount of a prodrug of a small molecule BRD9 degrader or a pharmaceutically acceptable salt thereof.
